SEQUENCE LISTING (2 sequences)

SEQ ID NO: 1 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 2 - protein, 194 aa, Homo sapiens [sequence omitted]

(19) United States
(12) Patent Application Publication
(10) Pub. No.: US 2026/0034094 A1
(43) Pub. Date: Feb. 5, 2026

(54) COMPOSITIONS AND METHODS FOR INDUCING FERROPTOSIS

(71) Applicant: Kojin Therapeutics, Inc., Boston, MA (US)

(72) Inventors: Laura Furst, Boston, MA (US); John Kittridge Eaton, JR., Boston, MA (US); John Hynes, JR., Boston, MA (US); Ramesh Kumar Sistla, Boston, MA (US); Lu Yan, Boston, MA (US)

(21) Appl. No.: 19/100,699

(22) PCT Filed: Aug. 2, 2023
(86) PCT No.: PCT/US2023/071521

§ 371 Date: Feb. 3, 2025

Related U.S. Application Data
(60) Provisional application No. 63/370,293, filed on Aug. 3, 2022.

(51) Int. Cl. A61K 31/397; A61K 31/197; A61K 31/337; A61K 31/351; A61K 31/36; A61K 31/381; A61K 31/404; A61K 31/415; A61K 31/4184; A61K 31/4192; A61K 31/4196; A61K 31/42; A61K 31/4245; A61K 31/426; A61K 31/4409; A61K 31/454; A61K 31/47; A61K 31/50; A61K 31/517; A61K 31/54; A61P 35/00; C07B 59/00; C07C 381/10; C07D 205/04; C07D 207/12; C07D 209/10; C07D 213/34; C07D 215/12; C07D 231/12; C07D 233/60; C07D 235/06; C07D 239/38; C07D 239/72; C07D 241/12; C07D 249/06; C07D 249/08; C07D 249/18; C07D 257/04; C07D 261/08; C07D 271/06; C07D 277/26; C07D 279/02; C07D 295/088; C07D 305/08; C07D 309/12; C07D 317/36; C07D 317/56; C07D 333/18; C07D 401/04; C07D 405/04; C07D 413/04

(52) U.S. Cl. CPC A61K 31/397; A61K 31/197; A61K 31/337; A61K 31/351; A61K 31/36; A61K 31/381; A61K 31/404; A61K 31/415; A61K 31/4184; A61K 31/4192; A61K 31/4196; A61K 31/42; A61K 31/4245; A61K 31/426; A61K 31/4409; A61K 31/454; A61K 31/47; A61K 31/50; A61K 31/517; A61K 31/54; A61P 35/00; C07B 59/001; C07B 59/002; C07C 381/10; C07D 205/04; C07D 207/12; C07D 209/10; C07D 213/34; C07D 215/12; C07D 231/12; C07D 233/60; C07D 235/06; C07D 239/38; C07D 239/72; C07D 241/12; C07D 249/06; C07D 249/08; C07D 249/18; C07D 257/04; C07D 261/08; C07D 271/06; C07D 277/26; C07D 279/02; C07D 295/088; C07D 305/08; C07D 309/12; C07D 317/36; C07D 317/56; C07D 333/18; C07D 401/04; C07D 405/04; C07D 413/04; C07B 2200/05

(57) ABSTRACT

Methods and systems and compounds and agents for the induction or modulation of ferroptosis in a cell, a tissue, or a tumor in a subject are provided. Various agents and compounds are described. Various methods of administration are described for optimal ferroptosis modulation, for instance, induction and killing of target cells. Methods of making, purifying, and characterizing various compounds are described.

### CROSS-REFERENCE

This application claims the benefit of U.S. Provisional Application No. 63/370,293, filed Aug. 3, 2022, the disclosures of which are incorporated herein by reference in their entirety.

### INCORPORATION BY REFERENCE OF SEQUENCE LISTING

The instant application contains a Sequence Listing, which has been submitted via patent Center. The Sequence Listing titled 203718-715601.xml, which was created on Jun. 29, 2023 and is 2,935 bytes in size, is hereby incorporated by reference in its entirety.

### BACKGROUND

Diseases such as cancer, autoimmune diseases, and fibrosis manifest when cells in the body exhibit uncontrolled, abnormal cell growth and proliferation. In order to treat, hyperproliferative diseases, the standard of care therapies induce cell death by a cellular process called apoptosis. The apoptosis pathway is engaged by many common types of anti-cancer therapies and ionizing radiation, which contributes to the regression of tumors or the toxic side effects of treatment. Given the ability for hyperproliferative cells to resist cell death by current apoptosis-driven therapeutics, there is a need for the development of new methods, compounds, and compositions of and for inducing cell death in hyperproliferative cells.

### SUMMARY

Provided herein is a compound of Formula I:

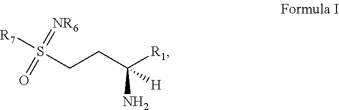

a diastereomer or enantiomer thereof, or a pharmaceutically acceptable salt of any of the foregoing, or a deuterated derivative of any of the forgoing,
wherein:
R₁ is:
   C(O)OH; or
   C(O)OX, wherein X is an organic cation, an inorganic cation, Na⁺, K⁺, Mg²⁺, Ca²⁺, Zn²⁺, or Mn²⁺; or

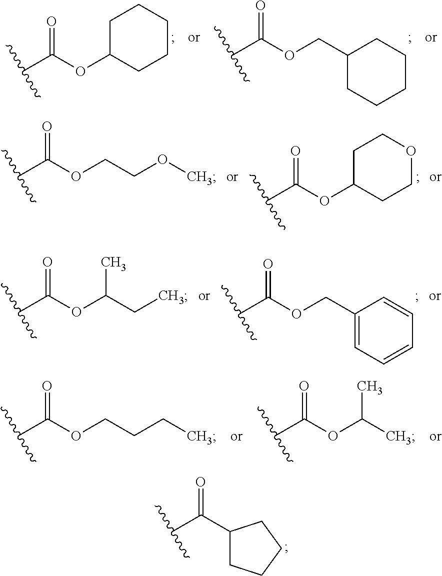

or C(O)OR₃, wherein R₃ is a linear or branched alkyl, a cycloaklyl, a cyclic ether, or a linear or branched alkyl ether, wherein any of these is optionally and independently substituted;
   or
   R₃ is

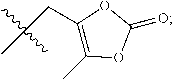 [SMILES: *Cc1oc(=O)oc1C]

or
R₃ is

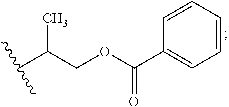 [SMILES: *C(C)COC(=O)c1ccccc1]

or R₁ is
C(O)N(R₄R₅)
   wherein R₄ is H; or
   R₄ is a linear or branched chain alkyl, cycloalkyl, heterocycloalkyl, aryl, or heteroaryl; wherein any of these is optionally and independently substituted; and
   R₅ is H; or
   R₅ is a linear or branched chain alkyl, cycloalkyl, heterocycloalkyl, aryl, or heteroaryl; wherein any of these is optionally and independently substituted; or
   R₅ is S(O)₂alkyl; or
   R₅ is S(O)₂CF₃; or
   R₅ is S(O)₂NH₂; or
   R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
   R₅ is

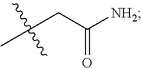 [SMILES: *CC(N)=O]

or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is

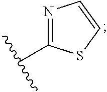 [SMILES: *c1nccs1]

or
R₅ is alkylaryl or benzyl; or
R₅ is

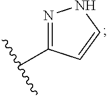 [SMILES: *c1cc[nH]n1]

or
R₅ is 2-pyridyl, 3-pyridyl; or 4-pyridyl; or
R₅ is

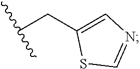 [SMILES: *Cc1cncs1]

or
R₅ is

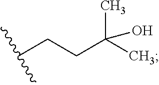 [SMILES: *CCC(C)(C)O]

or
R₅ is

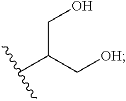 [SMILES: *C(CO)CO]

or
R₅ is

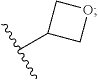 [SMILES: *C1COC1]

or
R₅ is

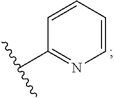 [SMILES: *c1ccccn1]

or
CN; or

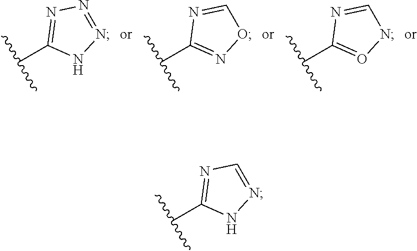

and
R₂ is: NH₂, NHC(O)OMe, or NHMe; and
R₆ is: H, C(O)Me, or P(O)(OH)₂; and
R₇ is: C₁-C₃ or C₅-C₁₀ linear or branched alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl, any of the foregoing can be independently and optionally substituted, wherein when the linear C₃-alkyl is substituted on a terminal carbon atom by a methyl group, the linear C₃-alkyl substituted on a terminal carbon atom by the methyl group contains a further substitution; wherein when the C₂-alkyl is substituted on a terminal carbon atom by an ethyl group, the C₂-alkyl substituted on a terminal carbon atom by an ethyl group contains a further substation; wherein when the C₁-alkyl is substituted on a terminal carbon atom by an n-propyl group, the C₁-alkyl substituted on a terminal carbon atom by an n-propyl group contains a further substation; or
R₇ is:

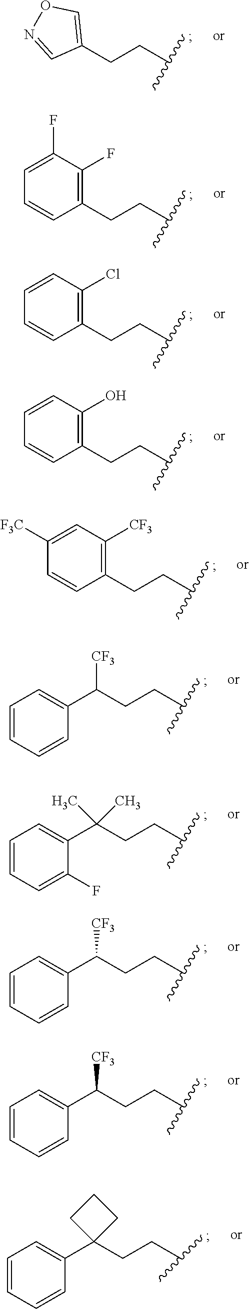

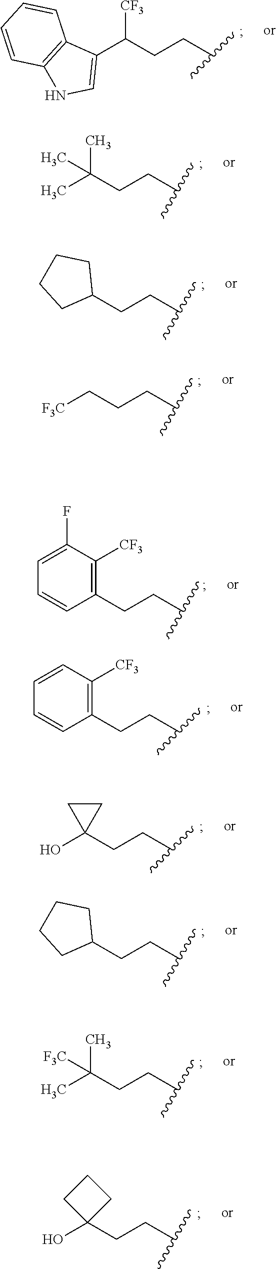

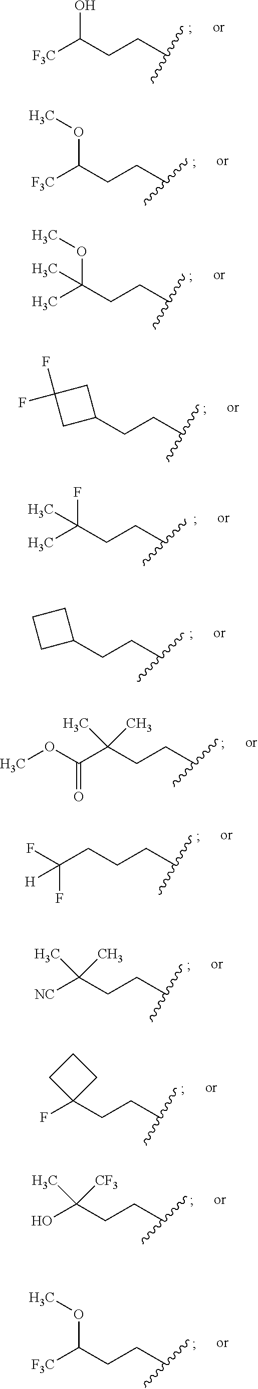

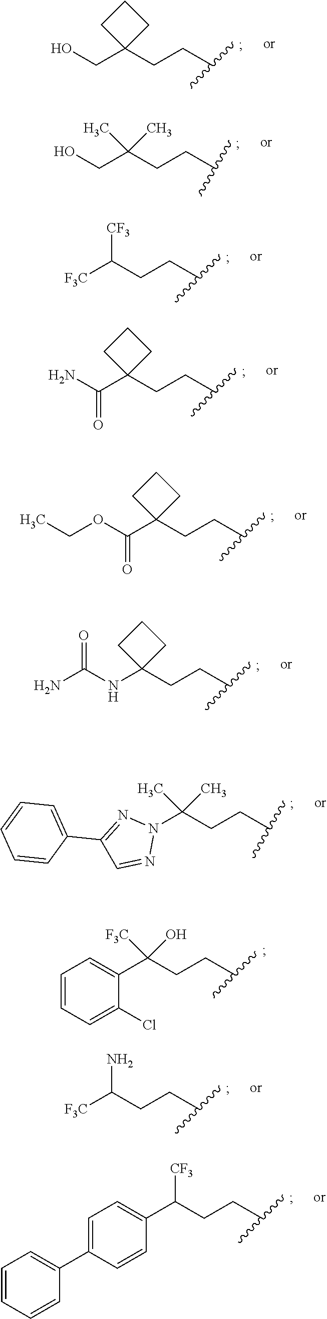

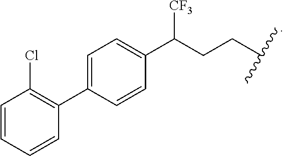 [SMILES: *CCC(c1ccc(-c2ccccc2Cl)cc1)C(F)(F)F]

In some instances, in a compound of Formula I, a diastereomer or enantiomer thereof, the pharmaceutically acceptable salt of any of the foregoing, or the deuterated derivative of any of the foregoing, R₁ is:
C(O)H; or
C(O)OH; or
C(O)OR₃,
where R₃ is a C₁-C₁₀ linear or branched alkyl, methyl, ethyl, n-propyl, iso-propyl, iso-butyl, sec-butyl, n-butyl, tert-butyl, pentyl, hexyl, heptyl, octyl, nonyl, C₁-C₁₀ cycloalkyl, cyclopropyl, cyclobutyl, cyclopentyl, cyclohexyl, cycloheptyl, alkylcycloalkyl, alkylcyclohexyl, methylcyclopropyl, methylcycobutyl, methylcyclopentyl, methylcyclohexyl, methylcycloheptyl, a linear or branched alkyl ether, 2-methoxyethyl, 3-methoxypropyl, 4-methoxybutyl, 5-methoxypropyl, cyclicalkylether, tetrahydropyranyl, 2-tetrahydropyranyl, 3-tetrahydropyranyl, 4-tetrahydropyranyl, tetrahydrofuranyl, 2-tetrahydrofuranyl, 2-tetrahydrofuranyl, alkylaryl, benzyl; and any of these is optionally and independently substituted with one or more C₁-C₁₀ alkyl, one or more halogens, one or more fluoro, one or more chloro, one or more deuterium, bromo, one or more iodo, aryl, C₆ aryl, C₁₀ aryl, heteroaryl, a C₁-C₇ alkylcycloaklyl, an unsubstituted tetrahydropyranyl, 2-tetrahydropyranyl, 3-tetrahydropyranyl, or 4-tetrahydropyranyl, or any combination thereof.

In some instances, in the compound of Formula I, a diastereomer or enantiomer thereof, the pharmaceutically acceptable salt of any of the foregoing, or the deuterated derivative of any of the foregoing
R₁ is C(O)N(R₄R₅)
   where R₅ is:
   C₁-C₁₀ a linear or branched chain alkyl, methyl, ethyl, propyl, or butyl; any of which are optionally or independently substituted by one or more deuterium, C₁-C₁₀ linear or branched chain alkyl, one or more halo, one or more fluoro, one or more chloro, one or more iodo, one or more or any combination thereof.

In some instances, in a compound of Formula I, a diastereomer or enantiomer thereof, the pharmaceutically acceptable salt of any of the foregoing, or the deuterated derivative of any of the foregoing,
R₁ is C(O)N(R₄R₅) and
   wherein R₅ is: heteroaryl, 2-pyridyl, 3-pyridyl, or 4-pyridyl; or
      R₅ is S(O)₂Me.

In some instances, in a compound of Formula I, a diastereomer or enantiomer thereof, the pharmaceutically acceptable salt of any of the foregoing, or the deuterated derivative of any of the foregoing
R₇ is: C₁-C₃ or C₅-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl; where any of foregoing can be independently and optionally substituted by one or more of a substituent that can be: deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an amino, a carboxylic acid or pharmaceutically acceptable salt thereof, an amide, a carbamate, a urea, an ester, an alkoxy, a methoxy, an ethoxy, a trifluoro methoxy, an ether, a cyclic ether, an C₁-C₇ alkyl ether, a C₁-C₇ cyclic alkyl ether, a trihalomethyl, a trifluoromethyl, an aryl, a heteroaryl, a fused aryl, a bi-aryl, a fused aryl-heteroaryl, a fused di-aryl, a fused aryl-heteroaryl, a 5-membered heteroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal; a ketal; or any combination of these;
Where:
when the linear C₃-alkyl is substituted on a terminal carbon atom by a methyl group substituent, the linear C₃-alkyl substituted on a terminal carbon atom by the methyl group substituent contains a further substitution;
when the C₂-alkyl is substituted on a terminal carbon atom by an ethyl group, the C₂-alkyl substituted on a terminal carbon atom by an ethyl group contains a further substitution;
when the C₁-alkyl is substituted on a terminal carbon atom by an n-propyl group, the C₁-alkyl substituted on a terminal carbon atom by an n-propyl group contains a further substation; and
where the C₁-C₁₀ linear alkyl substituent, the methyl substituent, the ethyl substituent, the C₁-C₁₀ branched chain alkyl substituent, the hydroxyl substituent, the amino substituent, the carboxylic acid or pharmaceutically acceptable salt thereof substituent, the amide substituent, the carbamate substituent, the urea substituent, the ester substituent, the alkoxy substituent, the methoxy substituent, the ethoxy substituent, the ether substituent, the cyclic ether substituent, the C₁-C₇ alkyl ether substituent, the C₁-C₇ cyclic ether substituent, the aryl substituent, the heteroaryl substituent, the fused aryl substituent, the bi-aryl substituent, the fused aryl-heteroaryl substituent, the fused di-aryl substituent, the fused aryl-heteroaryl substituent, the 5-membered heteroaryl substituent, the 6-membered heteroaryl substituent, the naphthyl substituent, the cycloalkyl substituent, the cyclopropyl substituent, the cyclobutyl substituent, the cyclopentyl substituent, the cyclohexyl substituent, the cycloheptyl substituent, the tert-butyl substituent, the bicyclic aliphatic substituent, the tricyclic aliphatic substituent, the adamantly substituent, or any combination of these can be independently and optionally substituted by one or more of:
a C₁-C₁₀ linear chain alkyl, a methyl, an ethyl, a C₁-C₁₀ branched chain alkyl, a halogen, a fluoro, a chloro, a bromo, an iodo, a hydroxyl, an alkoxy, a methoxy, an ethoxy, a carbamate, a urea, an amide, an ester, an amine, a trifluoro methoxy, an ether, an C₁-C₇ alkyl ether, a C₁-C₇ cyclic ether, a trihalomethyl, a trifluoromethyl, an aryl, a heteroaryl, a 5-membered hereroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal, a ketal, or any combination of these.

Also provided is a compound of Formula I:

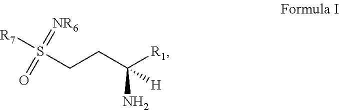

a diastereomer or enantiomer thereof, or a pharmaceutically acceptable salt of any of the foregoing, or a deuterated derivative of any of the forgoing,
wherein:
R₁ is:
   C(O)OH; or
   C(O)OX, wherein X is an organic cation, an inorganic cation, Na⁺, K⁺, Mg²⁺, Ca²⁺, Zn²⁺, or Mn²⁺; or

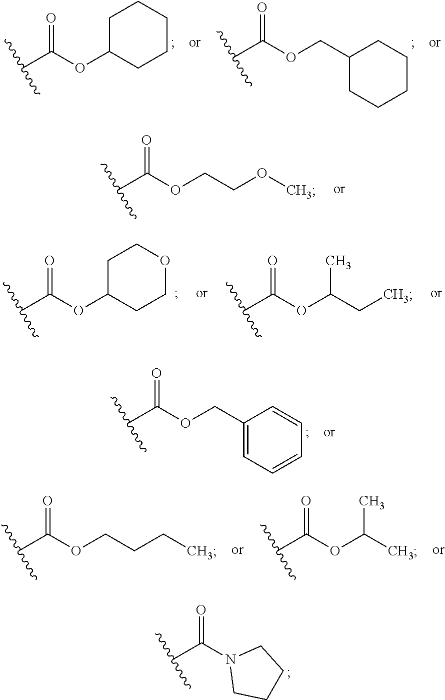

or
C(O)OR₃, wherein R₃ is a linear or branched alkyl, a cycloaklyl, a cyclic ether, or a linear or branched alkyl ether, wherein any of these is optionally and independently substituted;
   or
   R₃ is

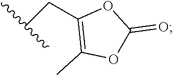 [SMILES: *Cc1oc(=O)oc1C]

or
R₃ is

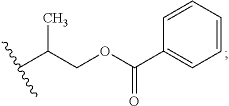 [SMILES: *C(C)COC(=O)c1ccccc1]

or
C(O)N(R₄R₅)
   wherein R₄ is H; or
   R₄ is a linear or branched chain alkyl, cycloalkyl, heterocycloalkyl, aryl, or heteroaryl; wherein any of these is optionally and independently substituted; and
   R₅ is H or absent; or
   R₅ is a linear or branched chain alkyl, cycloalkyl, heterocycloalkyl, aryl, or heteroaryl; wherein any of these is optionally and independently substituted; or
   R₅ is S(O)₂alkyl; or
   R₅ is S(O)₂CF₃; or
   R₅ is S(O)₂NH₂; or
   R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
   R₅ is

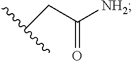 [SMILES: *CC(N)=O]

or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is

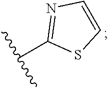 [SMILES: *c1nccs1]

or
R₅ is alkylaryl or benzyl; or
R₅ is

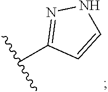 [SMILES: *c1cc[nH]n1]

or
R₅ is 2-pyridyl, 3-pyridyl; or 4-pyridyl; or
R₅ is

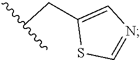 [SMILES: *Cc1cncs1]

or
R₅ is

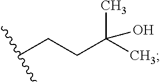 [SMILES: *CCC(C)(C)O]

or
R₅ is

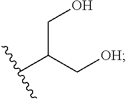 [SMILES: *C(CO)CO]

or
R₅ is

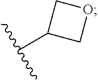 [SMILES: *C1COC1]

or
R₅ is

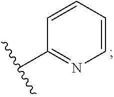 [SMILES: *c1ccccn1]

or
CN; or

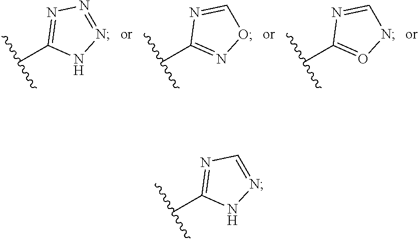

R₂ is: NH₂, NHC(O)OMe, or NHMe; and
R₆ is: H, C(O)Me, or P(O)(OH)₂; and
R₇ is: linear or branched chain: alkyl, alkenyl, or alkynyl, any of which can optionally and independently be substituted; or
R₇ is:

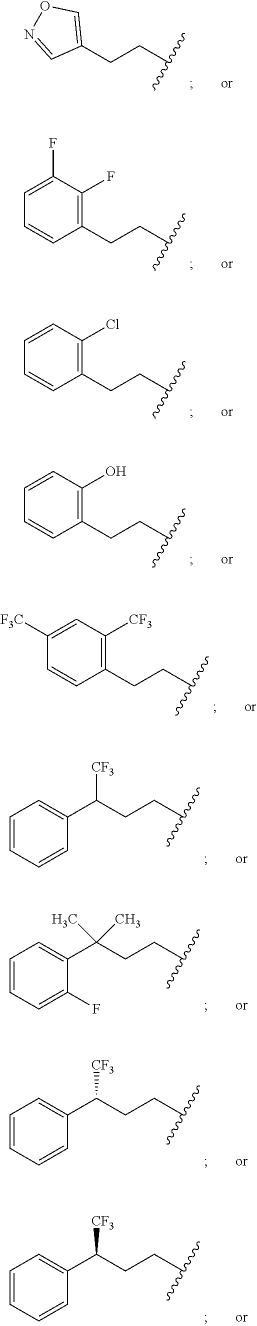

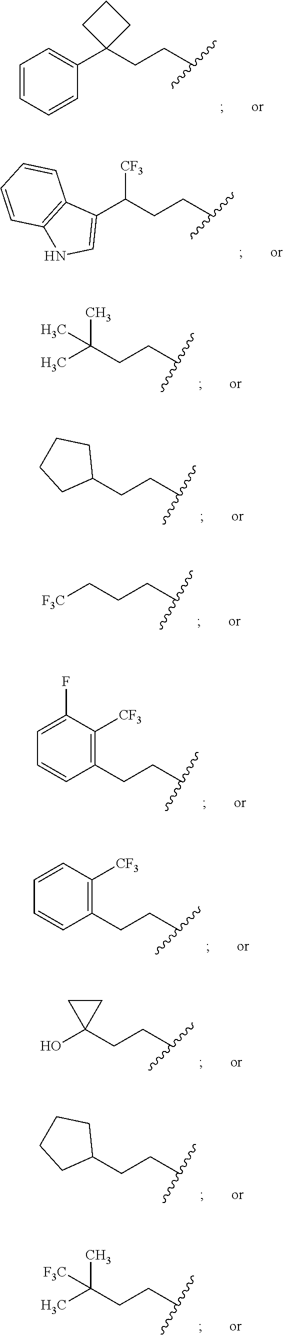

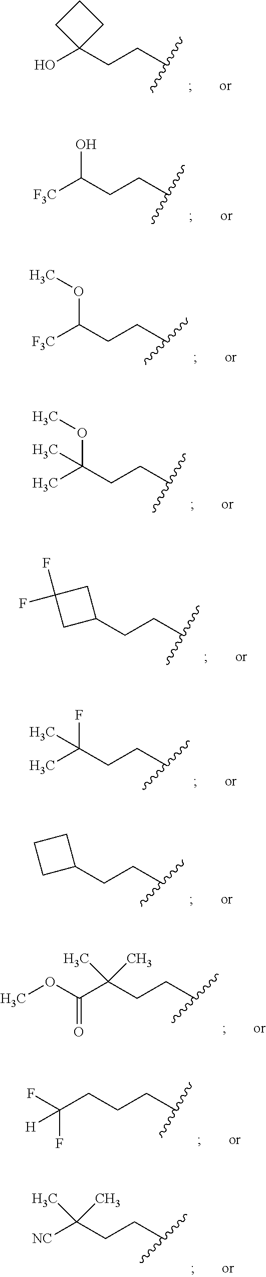

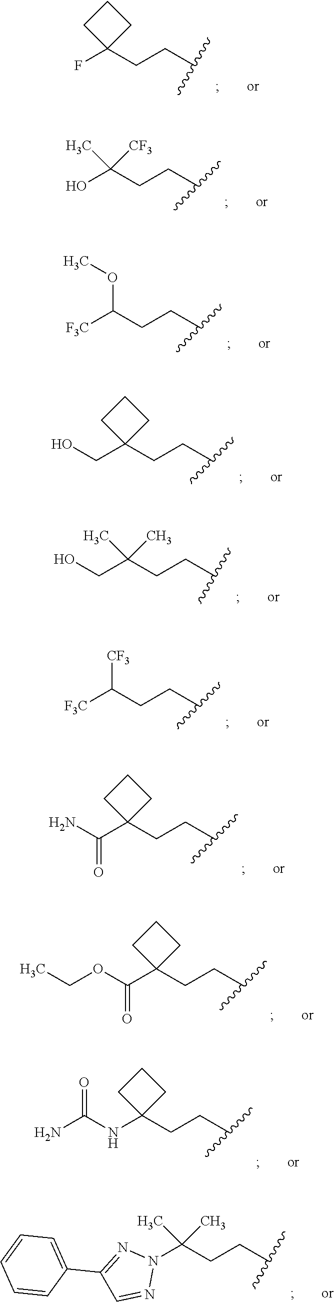

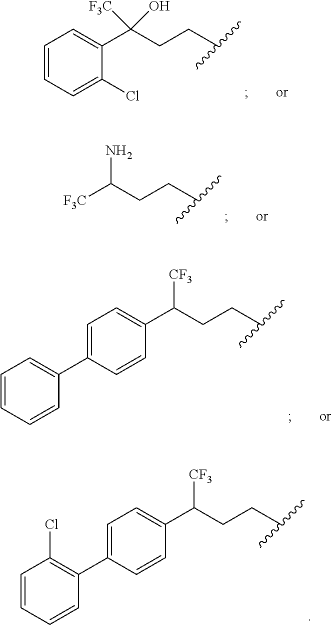 [SMILES: *CCC(O)(c1ccccc1Cl)C(F)(F)F ; *CCC(N)C(F)(F)F ; *CCC(c1ccc(-c2ccccc2)cc1)C(F)(F)F ; *CCC(c1ccc(-c2ccccc2Cl)cc1)C(F)(F)F]

and wherein the compound of Formula I is not buthionine sulfoximine (BSO) or a salt of BSO.

In some instances, in a compound of Formula I, a diastereomer or enantiomer thereof, the pharmaceutically acceptable salt of any of the foregoing, or the deuterated derivative of any of the foregoing,
R₁ is:
C(O)OH; or
C(O)OR₃, where R₃ is alkyl, C₁-C₁₀ linear or branched chain alkyl, methyl, ethyl, n-propyl, iso-propyl, iso-butyl, sec-butyl, n-butyl, tert-butyl, pentyl, hexyl, heptyl, octyl, nonyl, cycloalkyl, C₁-C₁₀ cycloalkyl, cyclopropyl, cyclobutyl, cyclopentyl, cyclohexyl, cycloheptyl, alkylcyclohexyl, methylcyclopropyl, methylcycobutyl, methylcyclopentyl, methylcyclohexyl, methylcycloheptyl, an alkyl ether, 2-methoxyethyl, 3-methoxypropyl, 4-methoxybutyl, 5-methoxypropyl, cyclicalkylether, tetrahydropyranyl, 2-tetrahydropyranyl, 3-tetrahydropyranyl, 4-tetrahydropyranyl, tetrahydrofuranyl, 2-tetrahydrofuranyl, 2-tetrahydrofuranyl, alkylaryl, benzyl; wherein any of these is optionally and independently substituted with one or more deuterium, C₁-C₁₀ linear or branched chain alkyl, one or more halogens, one or more fluoro, one or more chloro, one or more bromo, one or more iodo, aryl, C₆ aryl, C₁₀ aryl, heteroaryl, a C₁-C₇ alkylcycloaklyl, an unsubstituted tetrahydropyranyl, 2-tetrahydropyranyl, 3-tetrahydropyranyl, or 4-tetrahydropyranyl or any combination thereof.

In some instances, in the compound of Formula I, the diastereomer or enantiomer thereof, the pharmaceutically acceptable salt of any of the foregoing, or the deuterated derivative of any of the foregoing,
R₁ is C(O)N(R₄R₅).

In some instances, in the compound of Formula I, the diastereomer or enantiomer thereof, the pharmaceutically acceptable salt of any of the foregoing, or the deuterated derivative of any of the foregoing,
R₁ is C(O)N(R₄R₅) and
R₅ is: heteroaryl, 2-pyridyl, 3-pyridyl, or 4-pyridyl; or
R₅ is S(O)₂Me.

In some instances, in the compound of Formula I, the diastereomer or enantiomer thereof, the pharmaceutically acceptable salt of any of the foregoing, or the deuterated derivative of any of the foregoing,
R₇ is: C₁-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₁₀ linear or branched alkyl, the C₁-C₁₀ linear or branched chain alkenyl, or the C₁-C₁₀ linear or branched chain alkynyl; any of the foregoing can be independently substituted by one or more of a substituent that can be: deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an amino, a carboxylic acid or pharmaceutically acceptable salt thereof, an amide, a carbamate, a urea, an ester, an alkoxy, a methoxy, an ethoxy, a trifluoro methoxy, an ether, a cyclic ether, an C₁-C₇ alkyl ether, a C₁-C₇ cyclic alkyl ether, a trihalomethyl, a trifluoromethyl, an aryl, a heteroaryl, a fused aryl, a bi-aryl, a fused aryl-heteroaryl, a fused di-aryl, a fused aryl-heteroaryl, a 5-membered heteroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal; a ketal; or any combination of these;
where the C₁-C₁₀ linear alkyl substituent, the methyl substituent, the ethyl substituent, the C₁-C₁₀ branched chain alkyl substituent, the hydroxyl substituent, the amino substituent, the carboxylic acid or pharmaceutically acceptable salt thereof substituent, the amide substituent, the carbamate substituent, the urea substituent, the ester substituent, the alkoxy substituent, the methoxy substituent, the ethoxy substituent, the ether substituent, the cyclic ether substituent, the C₁-C₇ alkyl ether substituent, the C₁-C₇ cyclic ether substituent, the aryl substituent, the heteroaryl substituent, the fused aryl substituent, the bi-aryl substituent, the fused aryl-heteroaryl substituent, the fused di-aryl substituent, the fused aryl-heteroaryl substituent, the 5-membered heteroaryl substituent, the 6-membered heteroaryl substituent, the naphthyl substituent, the cycloalkyl substituent, the cyclopropyl substituent, the cyclobutyl substituent, the cyclopentyl substituent, the cyclohexyl substituent, the cycloheptyl substituent, the tert-butyl substituent, the bicyclic aliphatic substituent, the tricyclic aliphatic substituent, the adamantly substituent, or any combination of these can be independently and optionally substituted by one or more of:
deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an alkoxy, a methoxy, an ethoxy, a carbamate, a urea, an amide, an ester, an amine, a trifluoro methoxy, an ether, an C₁-C₇ linear or branched alkyl ether, a C₁-C₇ cyclic ether, a trihalomethyl, a trifluoromethyl, an aryl, a heteroaryl, a 5-membered hereroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal, a ketal, or any combination of these.

In some instances, in a compound of Formula I, a diastereomer or the enantiomer thereof, the pharmaceutically acceptable salt of any of the foregoing, or the deuterated derivative of any of the foregoing, R₁ is:

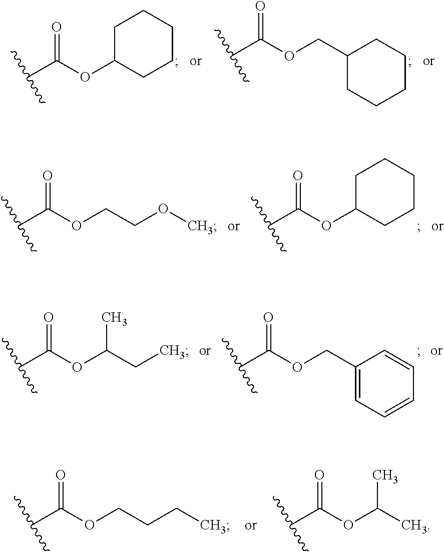

In some instances, in the compound of Formula I, the diastereomer or the enantiomer thereof, the pharmaceutically acceptable salt of any of the foregoing, or the deuterated derivative of any of the foregoing; R₁ is an ester or COOH.

In some instances, in the compound of Formula I, the diastereomer or the enantiomer of the foregoing, or the pharmaceutically acceptable salt of any of the foregoing, or the deuterated derivative of any of the foregoing; R₂ is NH₂.

In some instances, in the compound of Formula I, the diastereomer or the enantiomer of the foregoing, or the pharmaceutically acceptable salt of any of the foregoing; or the deuterated derivative of any of the foregoing; R₆ is H.

In some instances, in the compound of Formula I, the diastereomer or the enantiomer of the foregoing, or the pharmaceutically acceptable salt any of the foregoing, or the deuterated derivative of any of the foregoing; R₇ is

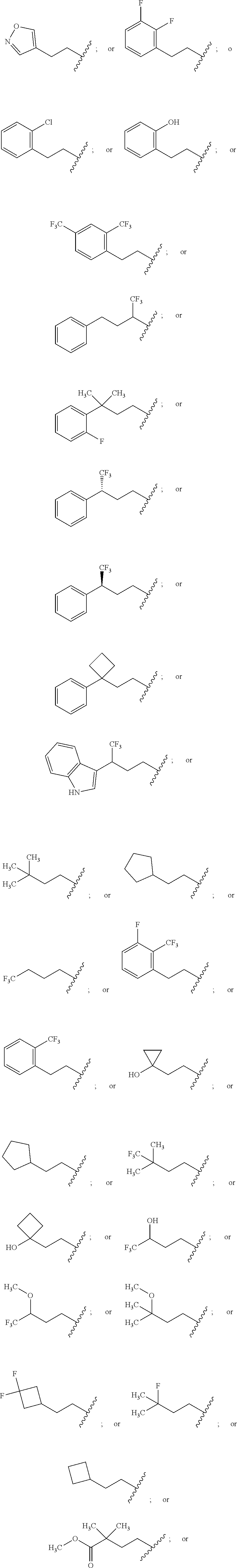

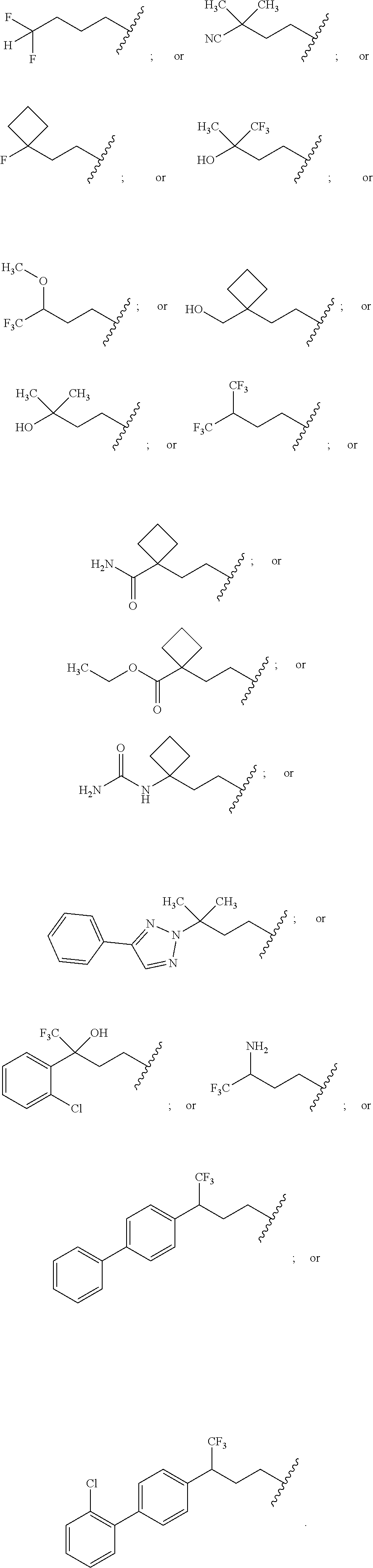

Also provided herein is a diastereomer or an enantiomer of the compound, or a pharmaceutically acceptable salt of any of the foregoing, or a deuterated derivative of any of the foregoing; wherein the compound is selected from the group consisting of

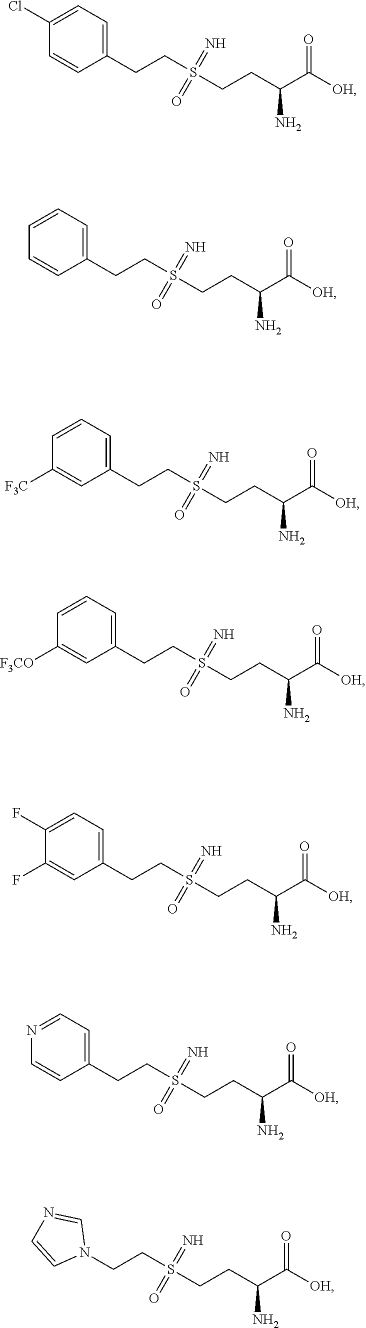

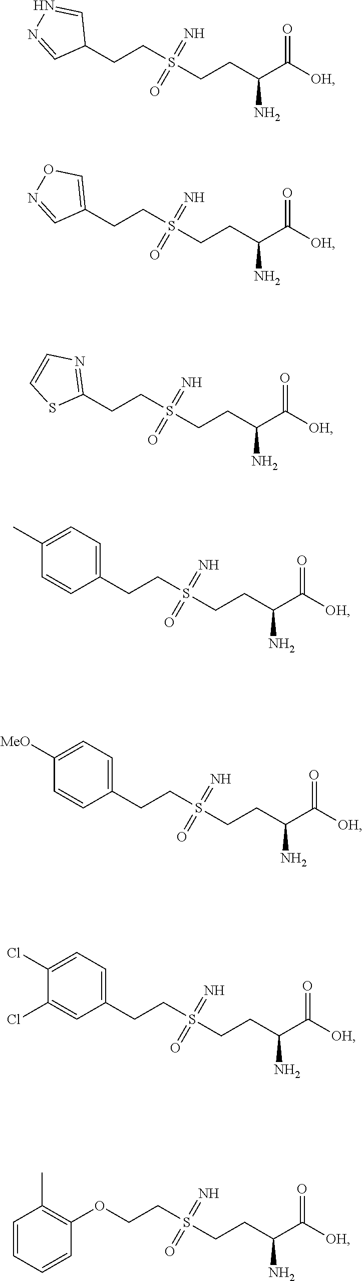

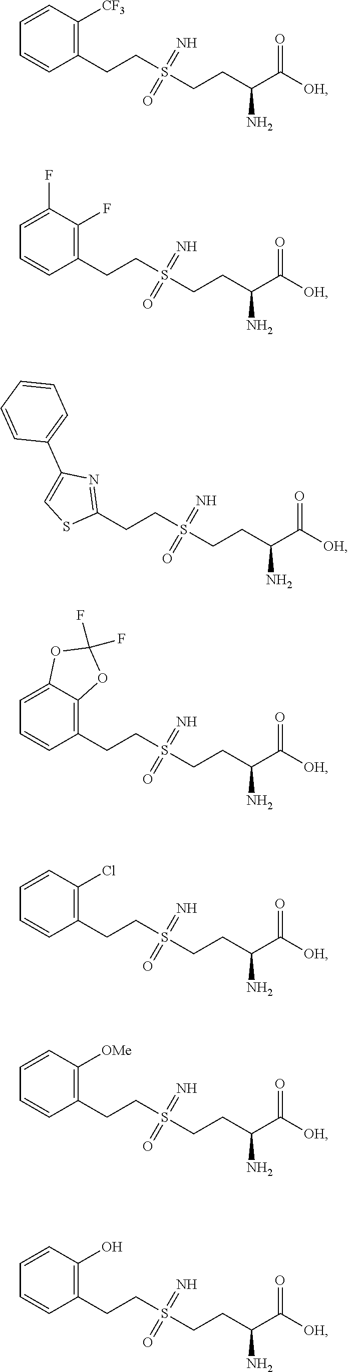

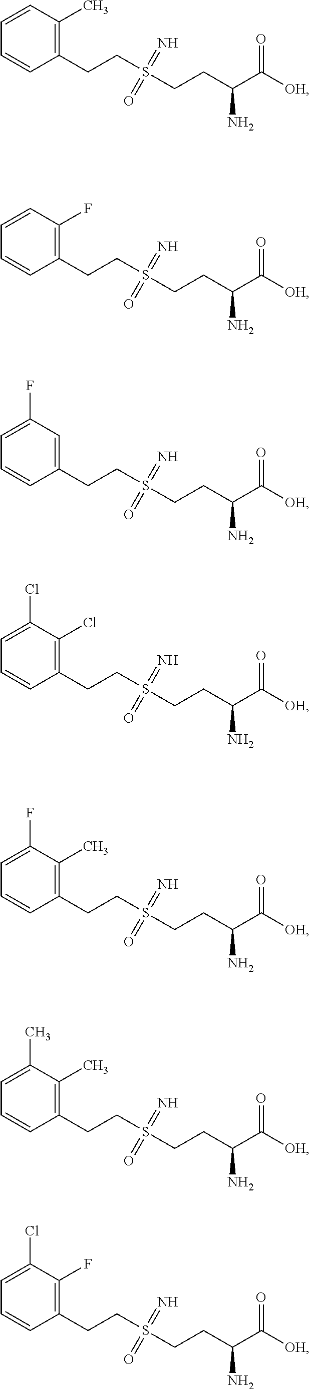

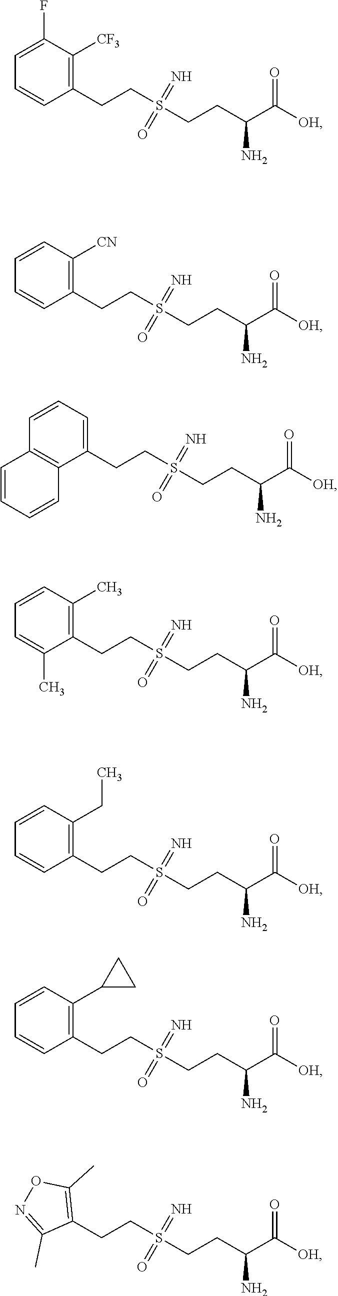

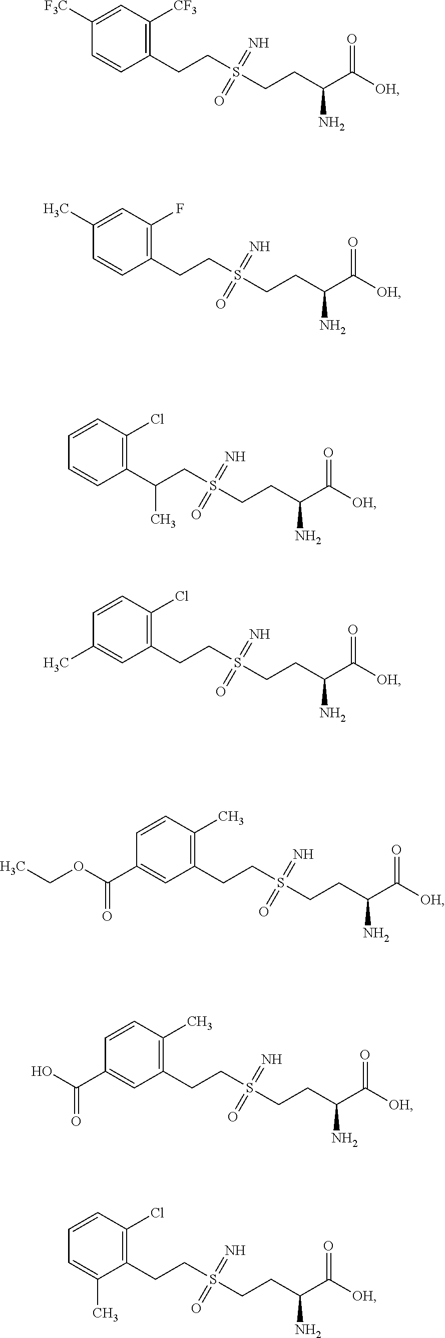

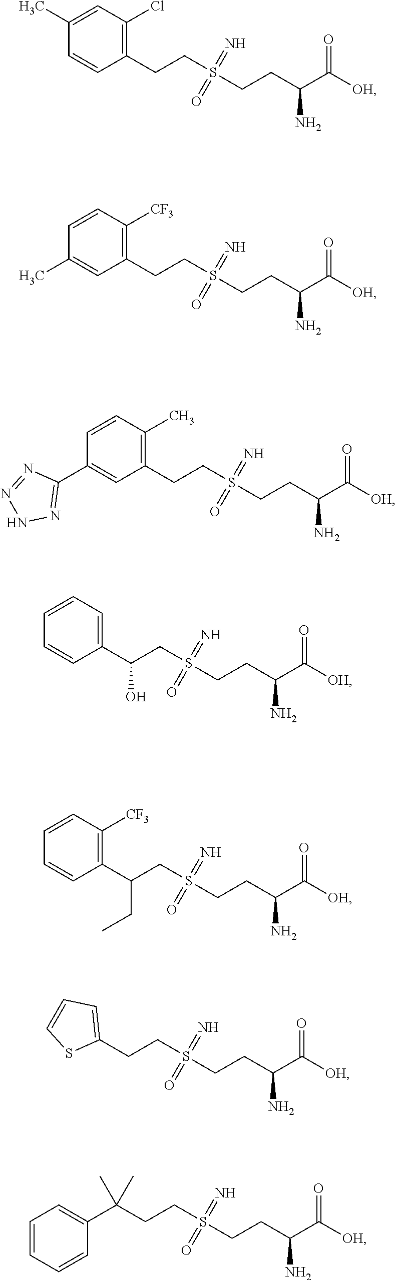

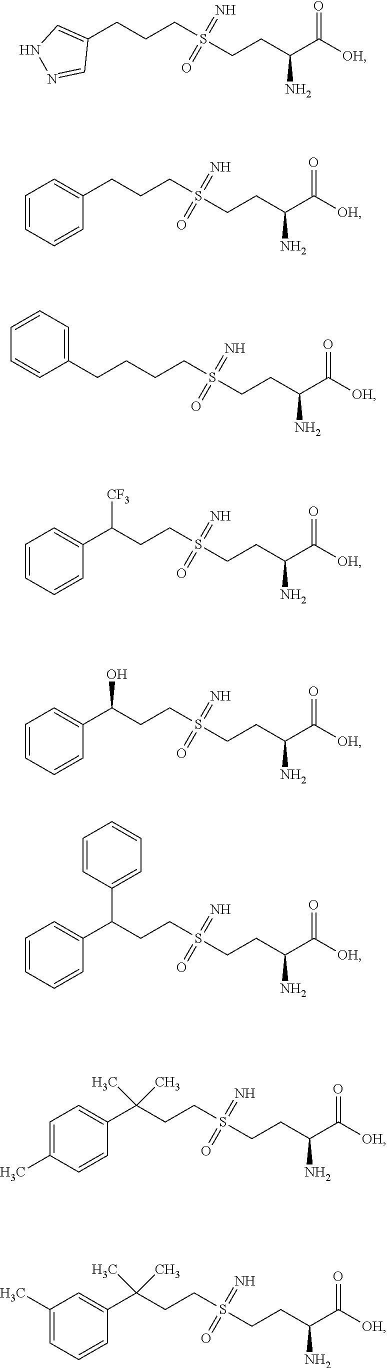

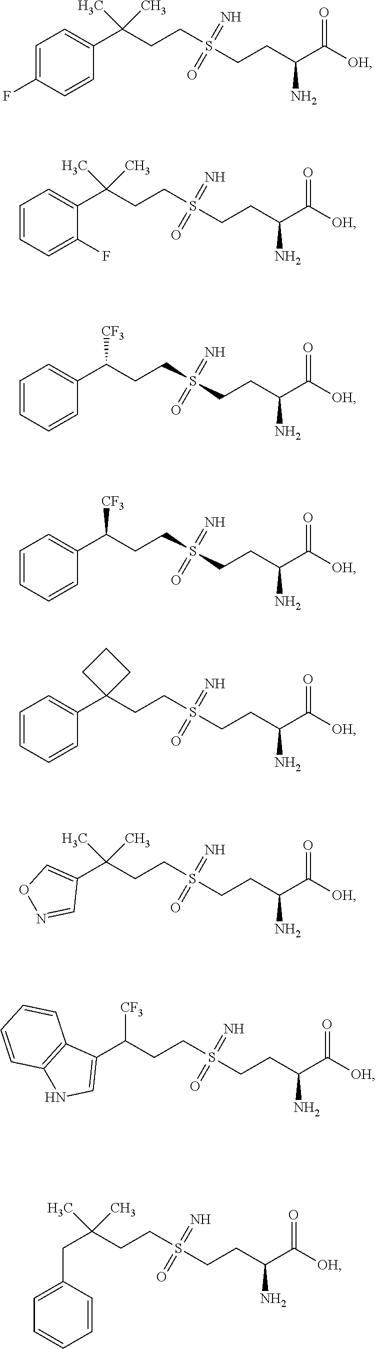

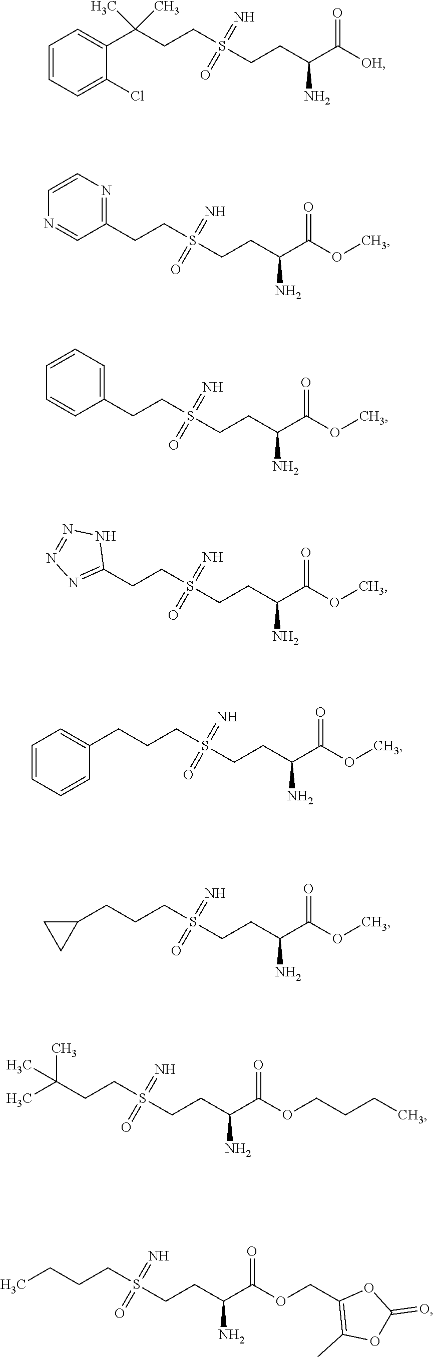

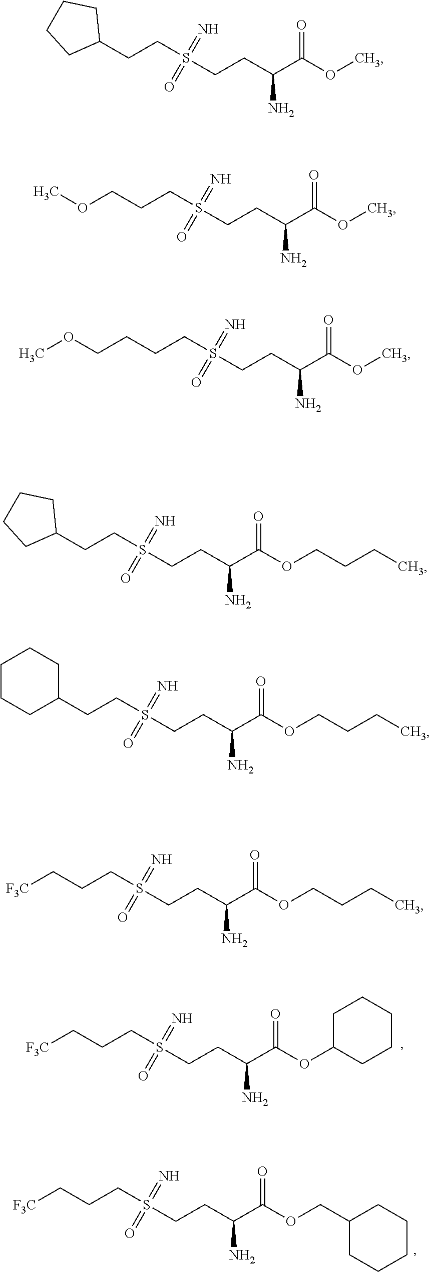

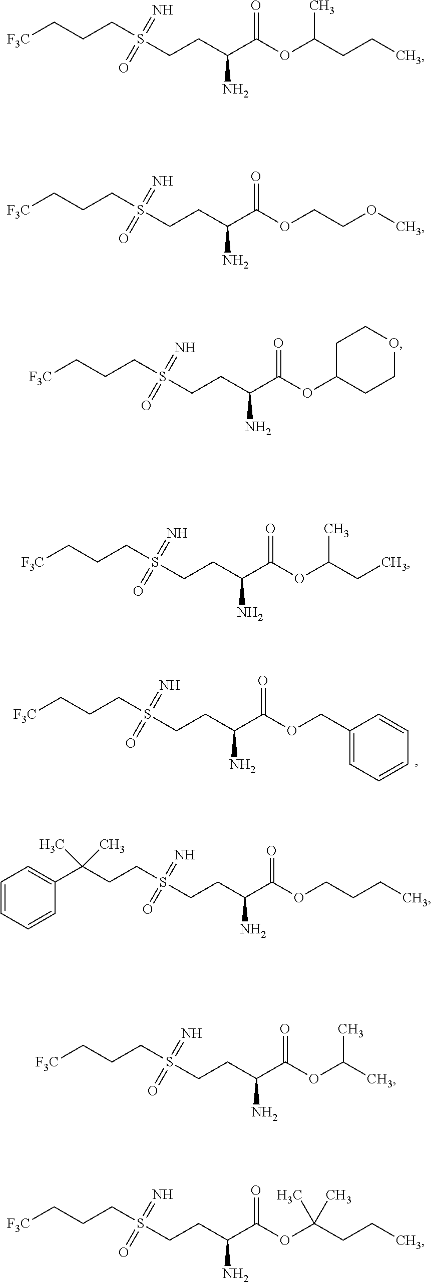

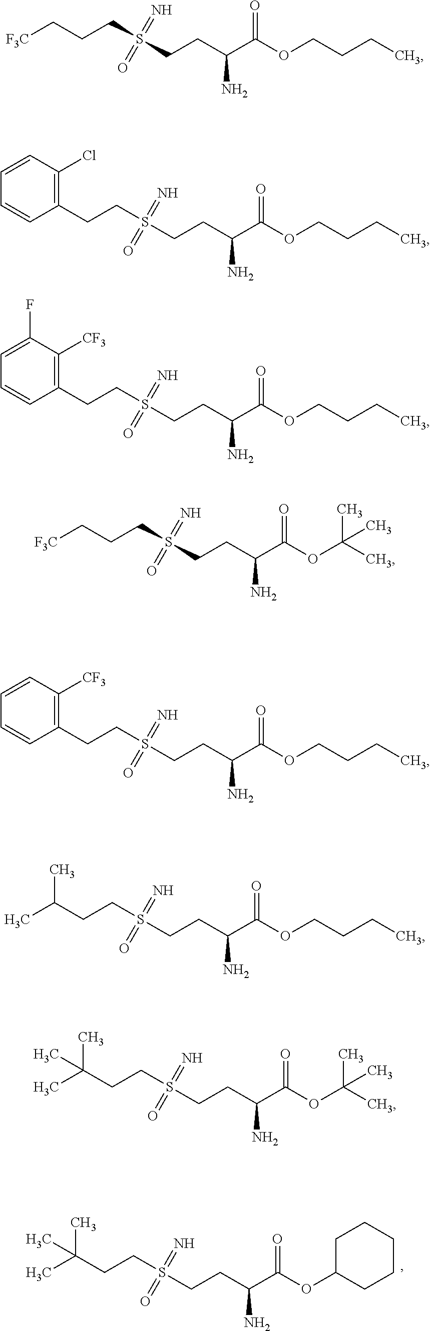

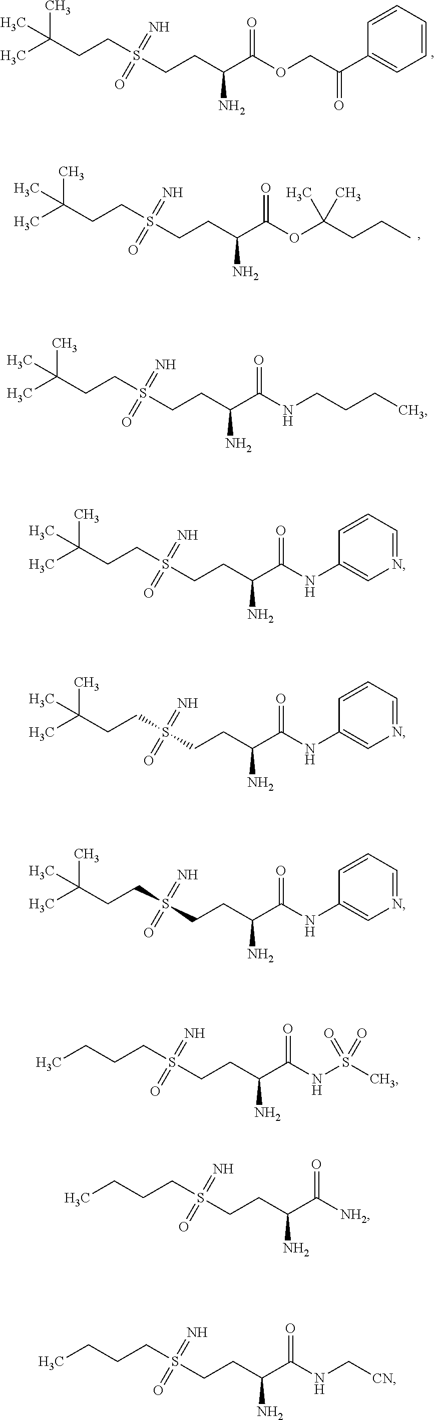

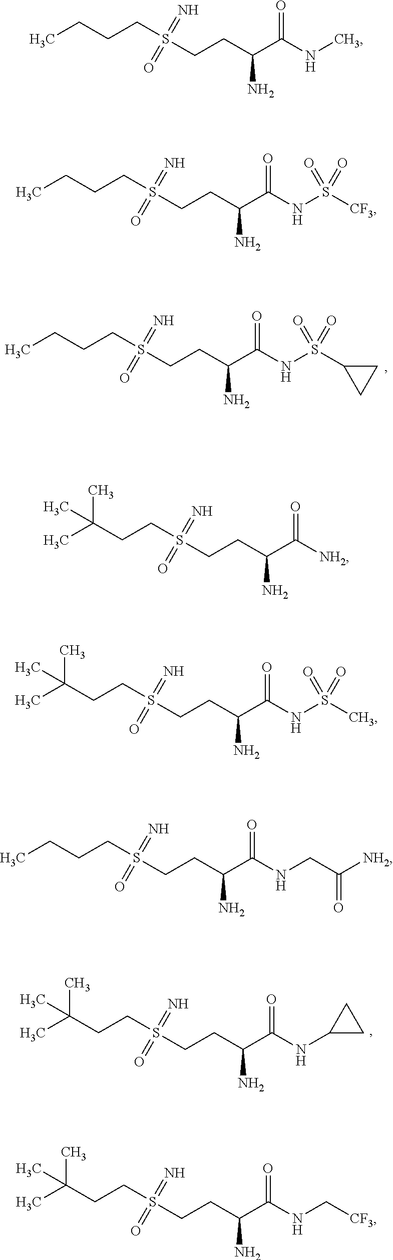

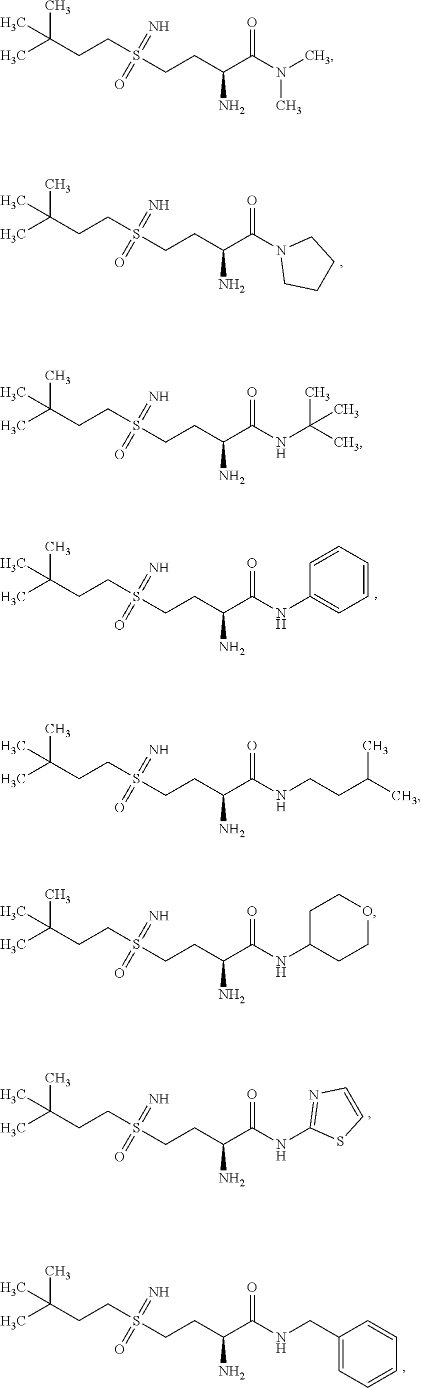

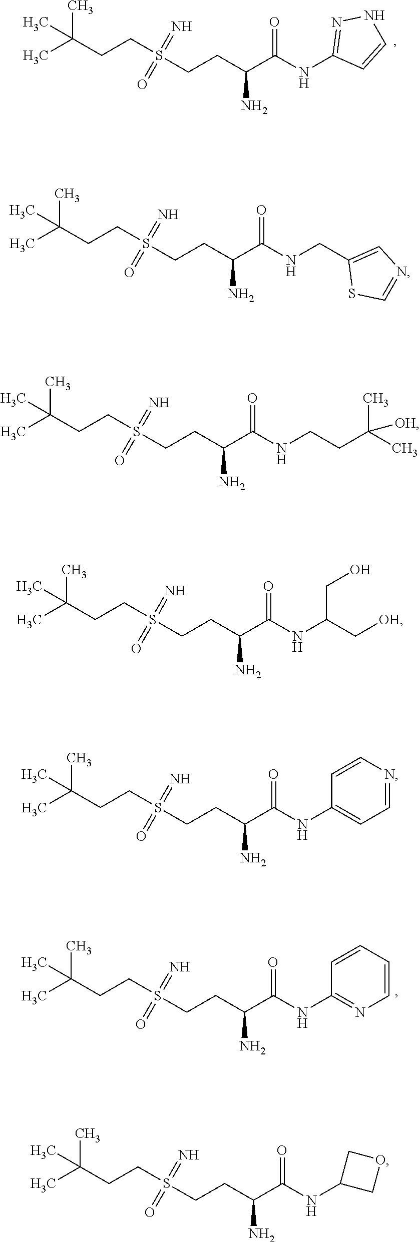

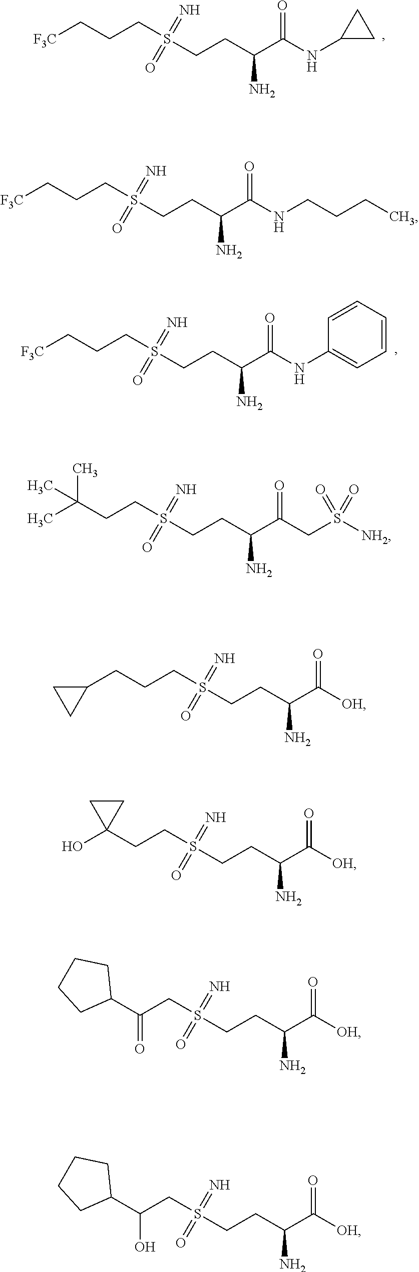

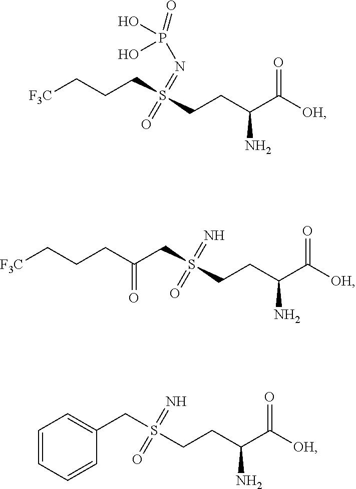 [SMILES: N[C@@H](CC[S@@](=O)(CCCC(F)(F)F)=NP(=O)(O)O)C(=O)O ; N=[S@@](=O)(CC[C@H](N)C(=O)O)CC(=O)CCCC(F)(F)F ; N=S(=O)(CC[C@H](N)C(=O)O)Cc1ccccc1]

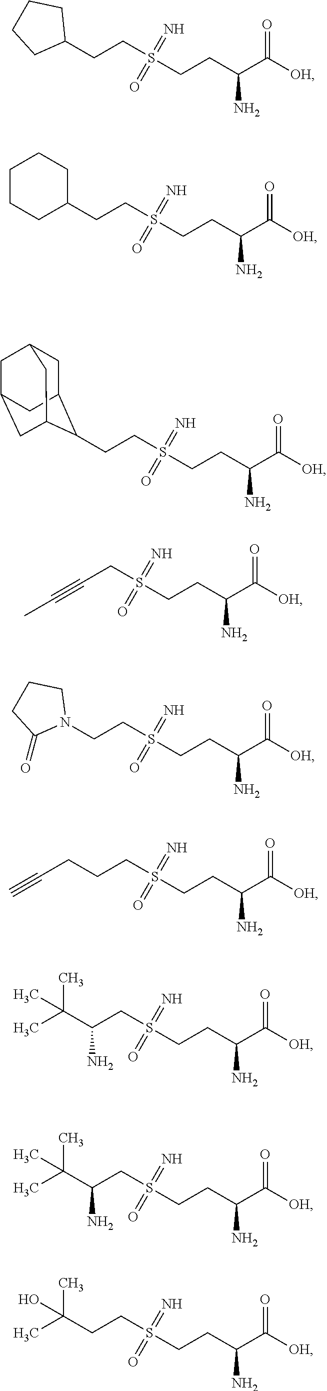

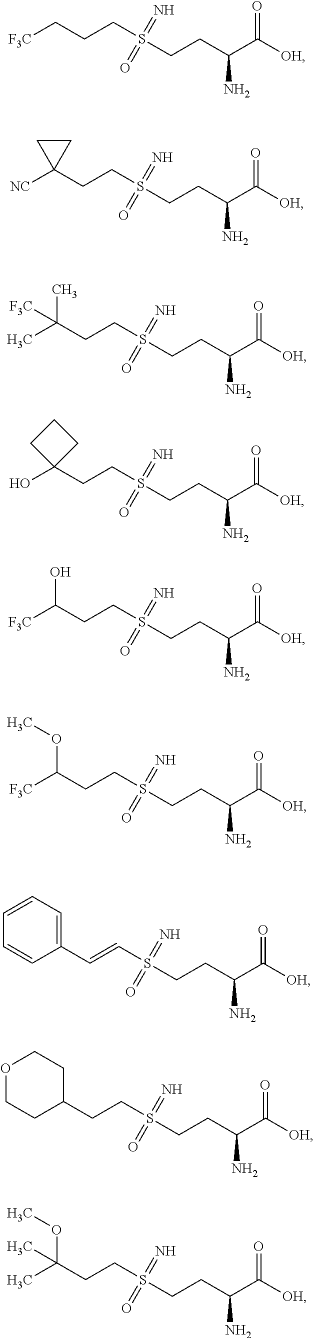

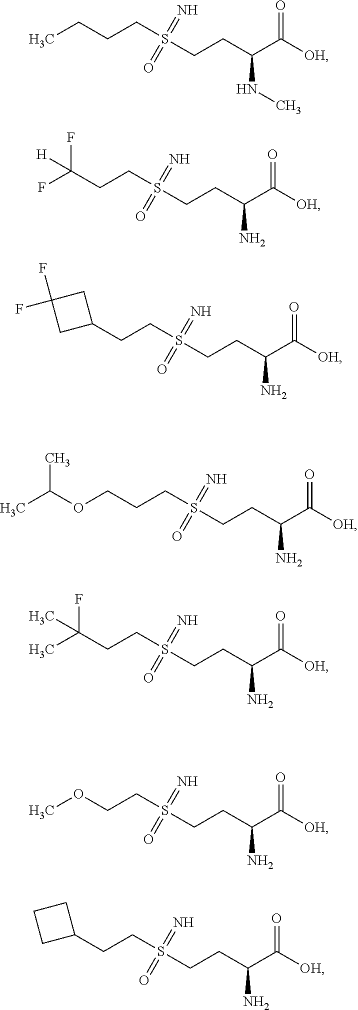

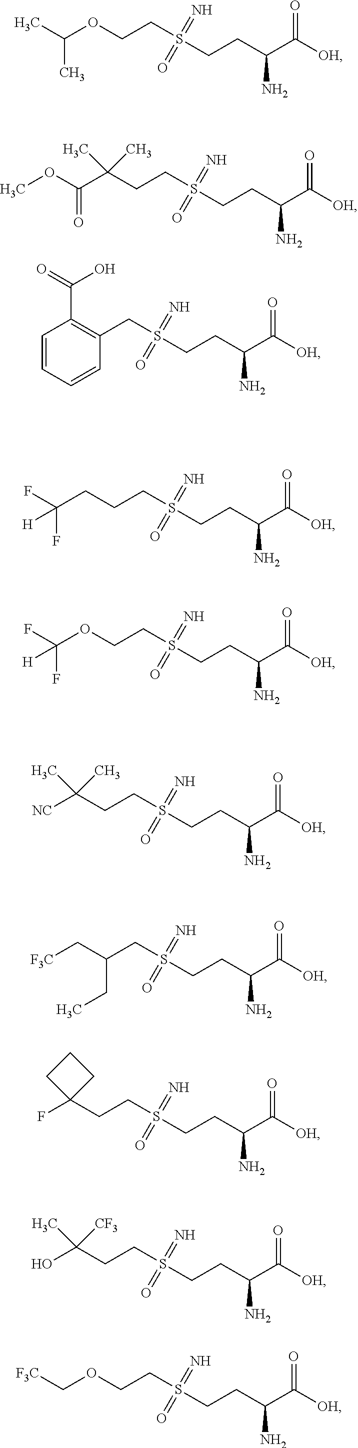

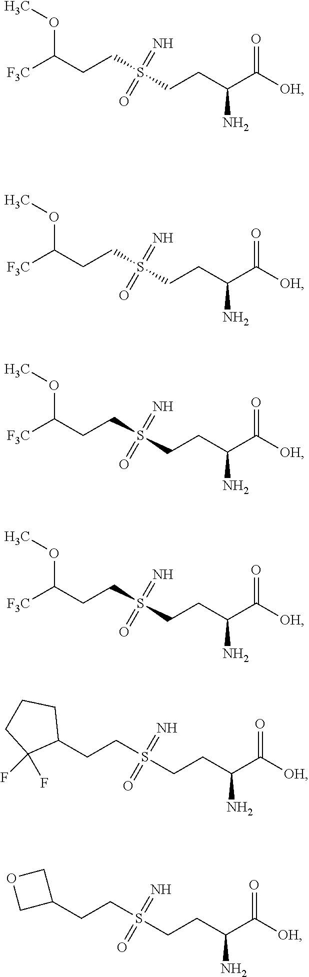 [SMILES: COC(CC[S@@](=N)(=O)CC[C@H](N)C(=O)O)C(F)(F)F ; COC(CC[S@@](=N)(=O)CC[C@H](N)C(=O)O)C(F)(F)F ; COC(CC[S@](=N)(=O)CC[C@H](N)C(=O)O)C(F)(F)F ; COC(CC[S@](=N)(=O)CC[C@H](N)C(=O)O)C(F)(F)F ; N=S(=O)(CCC1CCCC1(F)F)CC[C@H](N)C(=O)O ; N=S(=O)(CCC1COC1)CC[C@H](N)C(=O)O]

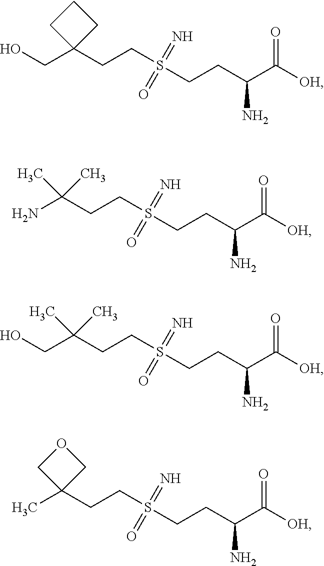 [SMILES: N=S(=O)(CC[C@H](N)C(=O)O)CCC1(CO)CCC1 ; CC(C)(N)CCS(=N)(=O)CC[C@H](N)C(=O)O ; CC(C)(CO)CCS(=N)(=O)CC[C@H](N)C(=O)O ; CC1(CCS(=N)(=O)CC[C@H](N)C(=O)O)COC1]

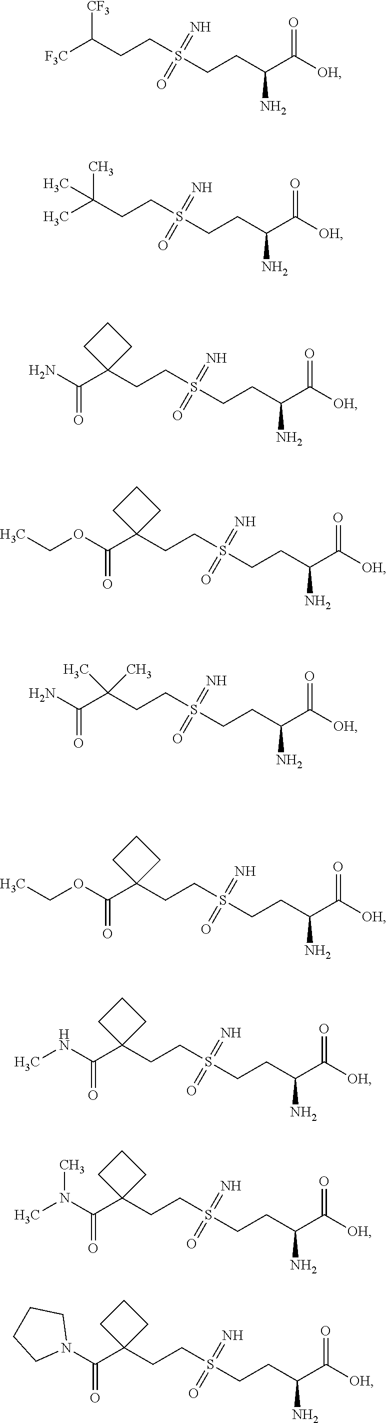

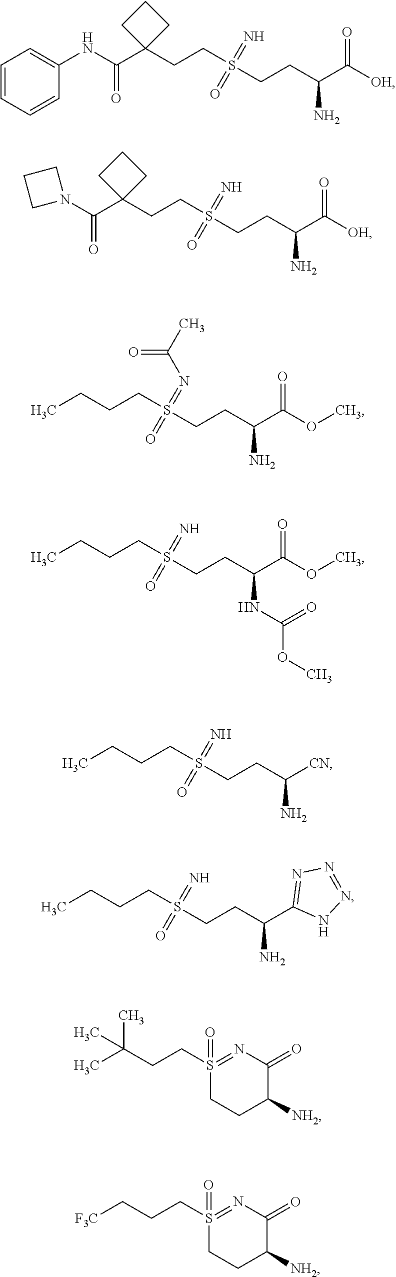

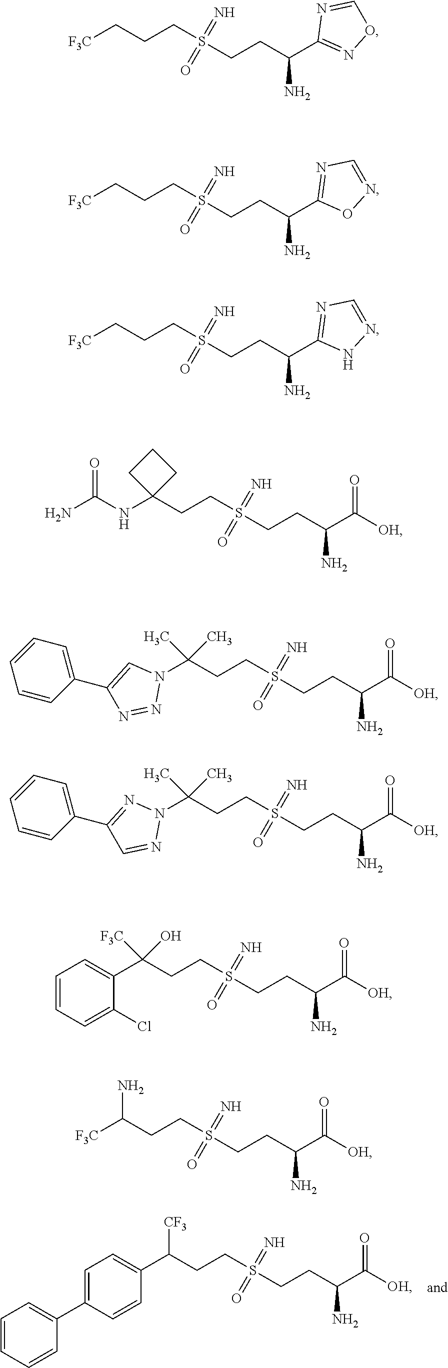

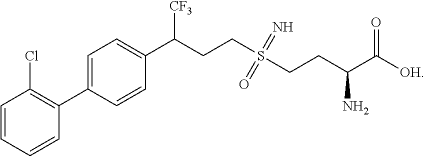 [SMILES: N=S(=O)(CCC(c1ccc(-c2ccccc2Cl)cc1)C(F)(F)F)CC[C@H](N)C(=O)O]

Also provided here is a compound, a diastereomer or an enantiomer of the compound, or a pharmaceutically acceptable salt of any of the foregoing, or a deuterated derivative of any of the foregoing; wherein the compound is selected from the group consisting of:

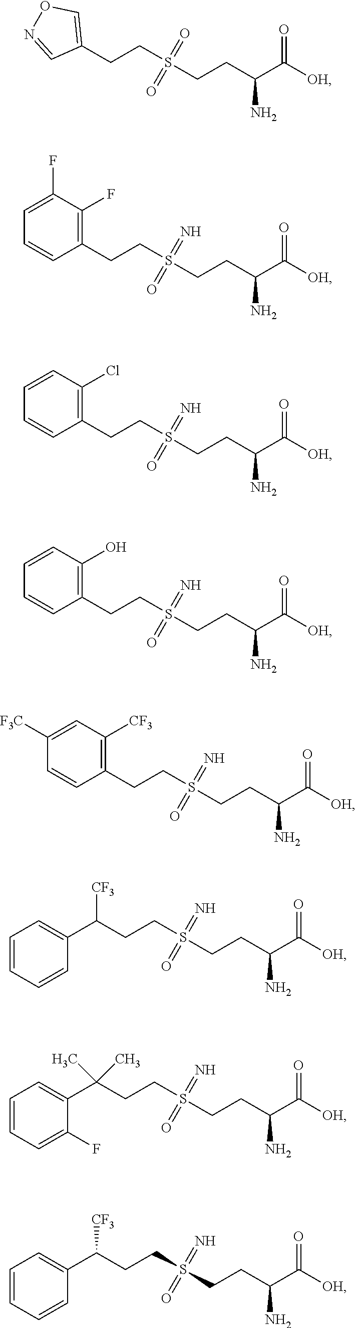

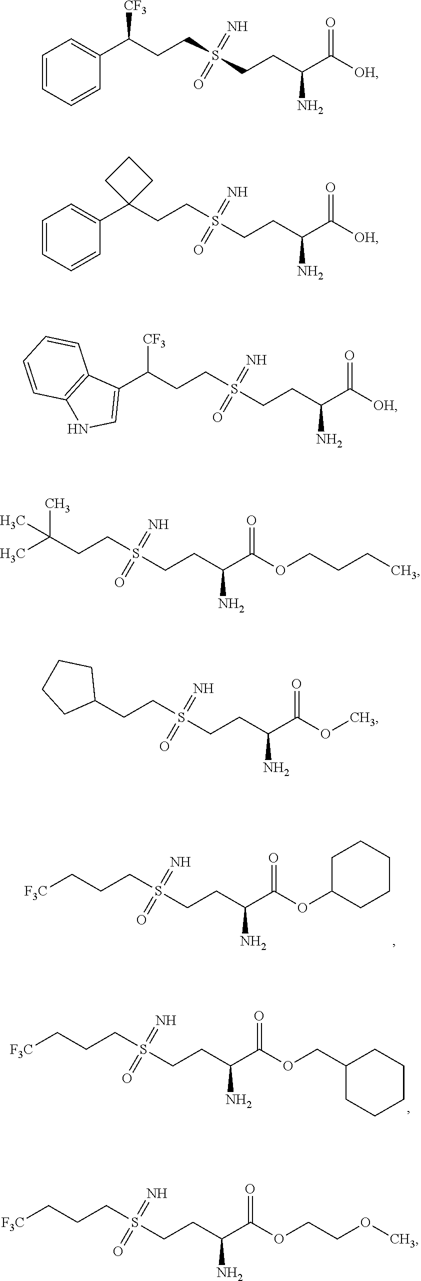

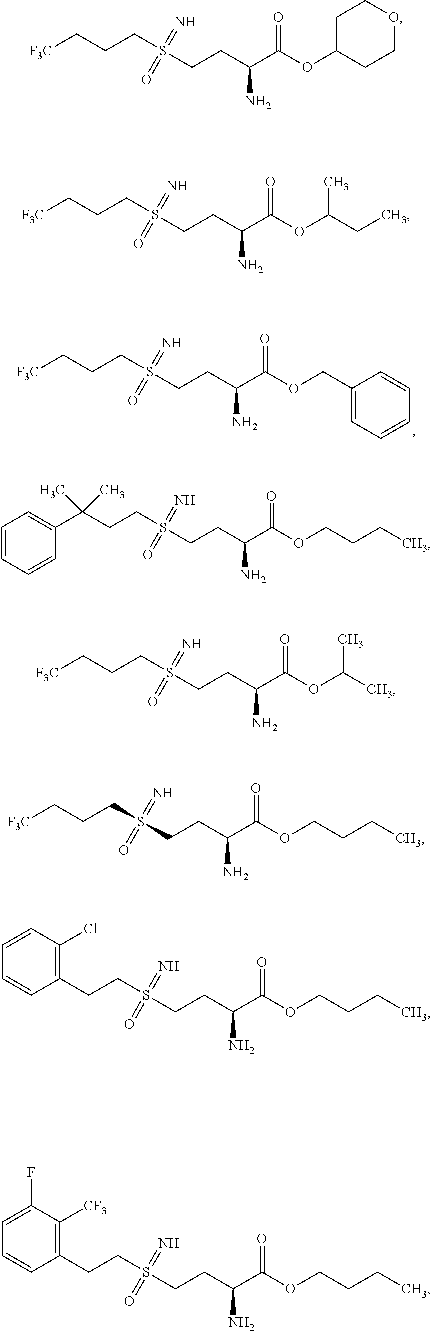

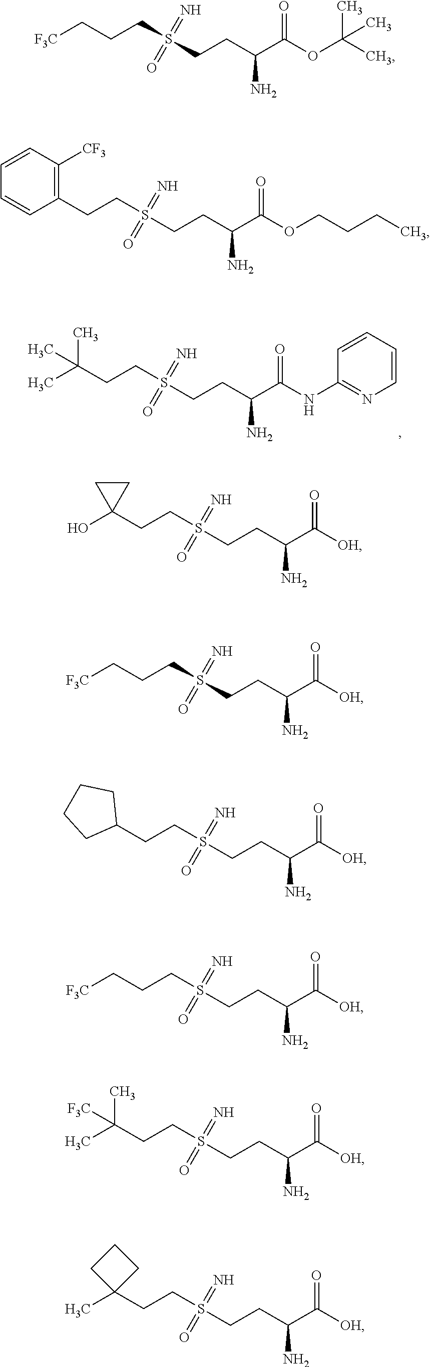

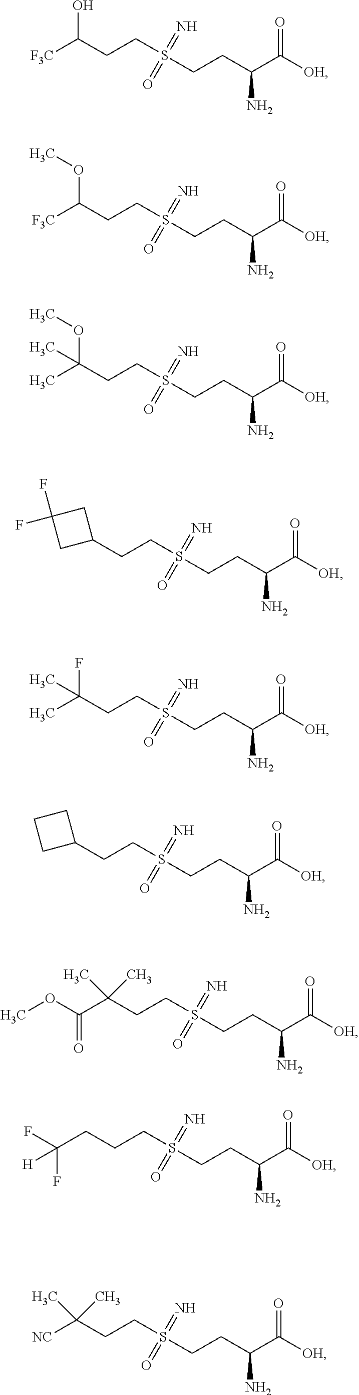

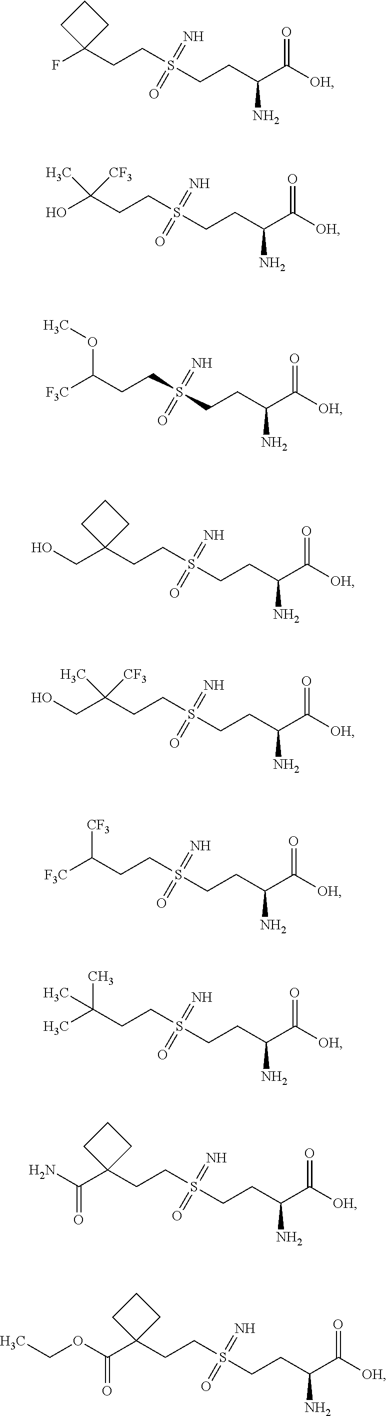

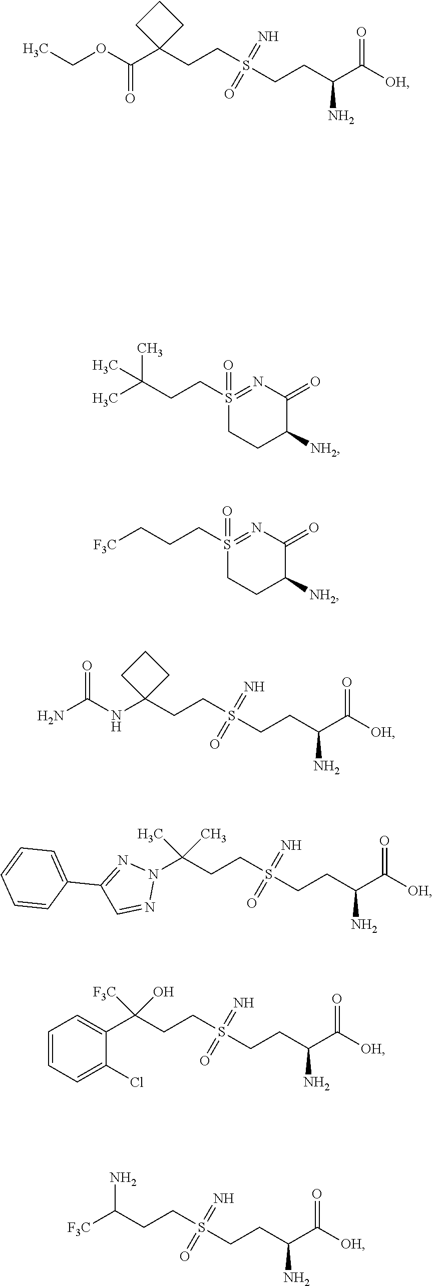

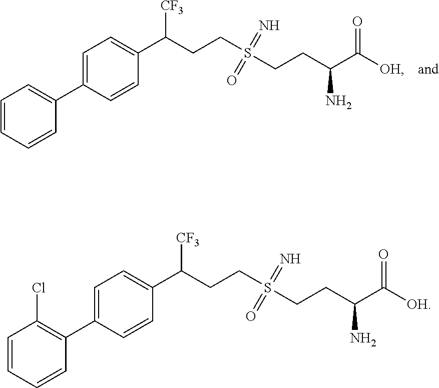 [SMILES: N=S(=O)(CCC(c1ccc(-c2ccccc2)cc1)C(F)(F)F)CC[C@H](N)C(=O)O ; N=S(=O)(CCC(c1ccc(-c2ccccc2Cl)cc1)C(F)(F)F)CC[C@H](N)C(=O)O]

Also provided herein is a compound of Formula II

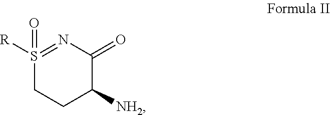

a diastereomer or an enantiomer of the compound of Formula II, or a pharmaceutically acceptable salt of any of the foregoing, or a deuterated derivative of any of the foregoing; wherein in the compound of Formula II, R is a: C₁, C₂, C₃, C₄, C₅, C₆, C₇, C₈, C₉, or C₁₀ linear or branched chain alkyl, optionally and independently substituted by a substituent selected from the group consisting of: a deuterium, a halogen, a fluorine, a chlorine, a C₁-C₁₀ linear or branched chain alkyl, a methyl, an ethyl, a propyl, an iso-propyl, a butyl, an isobutyl, a sec-butyl, a tert-butyl, and any combination of these.

Also provided herein is a pharmaceutical composition comprising the compound of Formula I, the compound of Formula II, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. The pharmaceutical composition can be in unit dose form. Additionally, the pharmaceutical composition can comprise an additional active agent or pharmaceutically acceptable salt thereof or prodrug thereof. In some embodiments, the prodrug is an ester. In some embodiments, the ester is an ethyl ester or a tert-butyl ester. Further, the pharmaceutical composition can be in the form of a powder, a tablet, a capsule, a liquid, or a gel. In some embodiments, in the pharmaceutical composition, the compound of Formula I, the compound of Formula II, the compound; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg. In some embodiments, the additional active agent or pharmaceutically acceptable salt thereof or prodrug thereof can be independently present in the pharmaceutical composition in an amount ranging from about 0.001 mg to about 25,000 mg.

Also provided herein is a pharmaceutical composition comprising the compound of Formula XVIII, the compound of Formula XIX, the compound of Formula XX, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. The pharmaceutical composition can be in unit dose form. Additionally, the pharmaceutical composition can comprise an additional active agent or pharmaceutically acceptable salt thereof or prodrug thereof. In some embodiments, the prodrug is an ester. In some embodiments, the ester is an ethyl ester or a tert-butyl ester. Further, the pharmaceutical composition can be in the form of a powder, a tablet, a capsule, a liquid, or a gel. In some embodiments, in the pharmaceutical composition, the compound of Formula I, the compound of Formula II, the compound; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg. In some embodiments, the additional active agent or pharmaceutically acceptable salt thereof or prodrug thereof can be independently present in the pharmaceutical composition in an amount ranging from about 0.001 mg to about 25,000 mg.

In some embodiments is provided a kit comprising a compound therein, a diastereomer thereof, an enantiomer thereof, a pharmaceutically acceptable salt thereof, or a deuterated derivative thereof, or a pharmaceutically composition therein, and a container. In some embodiments are pharmaceutical compositions described herein, and a container. In some embodiments, the container is a syringe. In some embodiments, the container is an intravenous (IV) bag. In some embodiments, the container is disposable. In some embodiments, the container is recyclable. In some embodiments, the container is a single use container. In some embodiments, the container is resealable.

In some embodiments is provided a method of treating a disease or condition in a subject. In some embodiments, the disease or condition is a cancer. In some embodiments, the method comprises administering a therapeutically effective amount of the pharmaceutical composition herein to the subject, who can be a subject in need thereof, thereby treating the disease or the condition, which can be a cancer. In some embodiments is provided a method of treating a disease or condition in a subject, who can be subject in need thereof, the method comprising administering the compound of Formula I, the compound of Formula II, the diastereomer or the enantiomer of any of the foregoing, or the pharmaceutically acceptable salt of any of the foregoing, or the deuterated derivative of any of the foregoing; in a therapeutically effective amount to the subject, thereby treating the disease or condition, which can be a cancer. In some embodiments, the administering is selected from the group consisting of: oral, an injection; subcutaneous, intra-tumoral; systemic, local, intravenous, intraperitoneal, intramuscular, and any combination thereof. In some embodiments, the subject can be a mammal. In some embodiments, the subject can be a human. In some embodiments, the subject can be a male. In some embodiments, the subject can be a female.

In some embodiments is provided a method of modulating ferroptosis in a tissue, which can be in a subject, which can be a subject in need thereof, the method comprising contacting, for example directly or indirectly, optionally in a sustained manner, the tissue with a pharmaceutical composition herein in an amount effective to modulate the ferroptosis in the tissue. In some embodiments, the subject can be a human. In some embodiments, the subject can be a male. In some embodiments, the subject can be a female.

In some embodiments, in a method herein, the administering or the contacting can be: as needed, once per day, twice per day, three times per day, once per week, once per two weeks, once per three weeks, once per month, once every six months, once per year, or for life. In some embodiments, an effective or a therapeutically effective amount can range from about 0.001 mg to about 25,000 mg of a compound herein, an enantiomer or a diastereomer thereof, a pharmaceutically acceptable salt of any of these, or a deuterated derivative of any of these, or of a pharmaceutical composition herein, which can optionally be in unit dose form.

In some embodiments, also are provided methods of making and testing compounds of Formula I and Formula II, enantiomers and diastereomers of any of these, salts and pharmaceutically acceptable salts of any of these, and deuterated derivatives of any of these.

In some embodiments is a compound, a diastereomer or an enantiomer of the compound, or a pharmaceutically acceptable salt of any of the foregoing, or a deuterated derivative of any of the foregoing; wherein the compound is selected from the group consisting of:

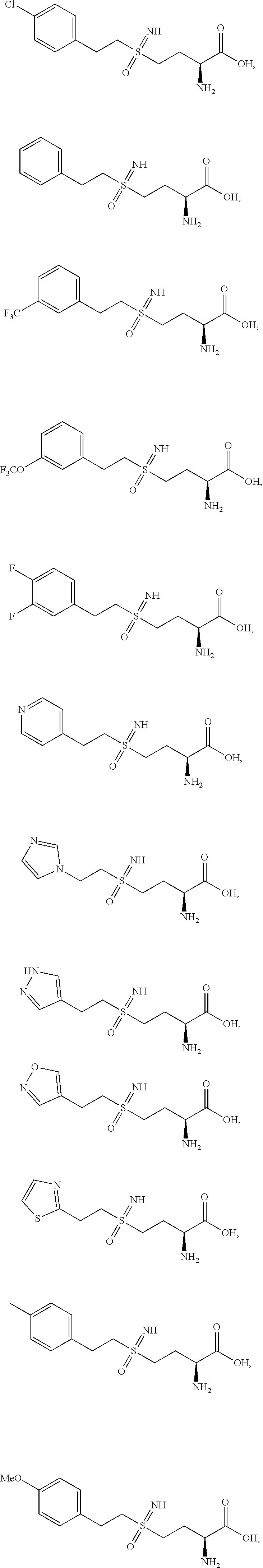

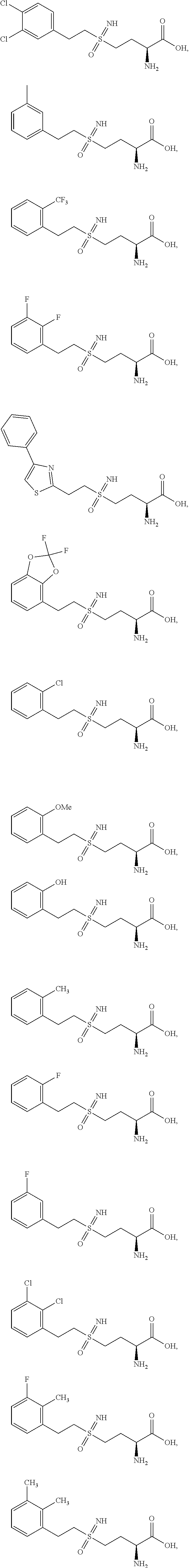

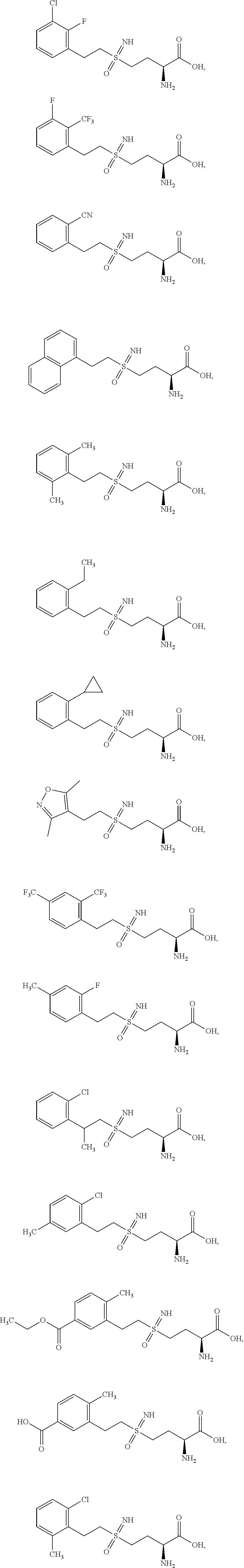

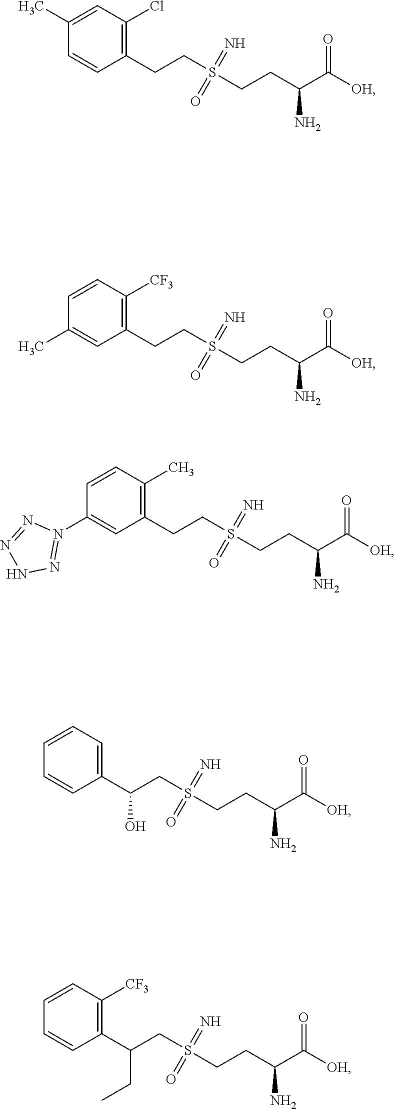

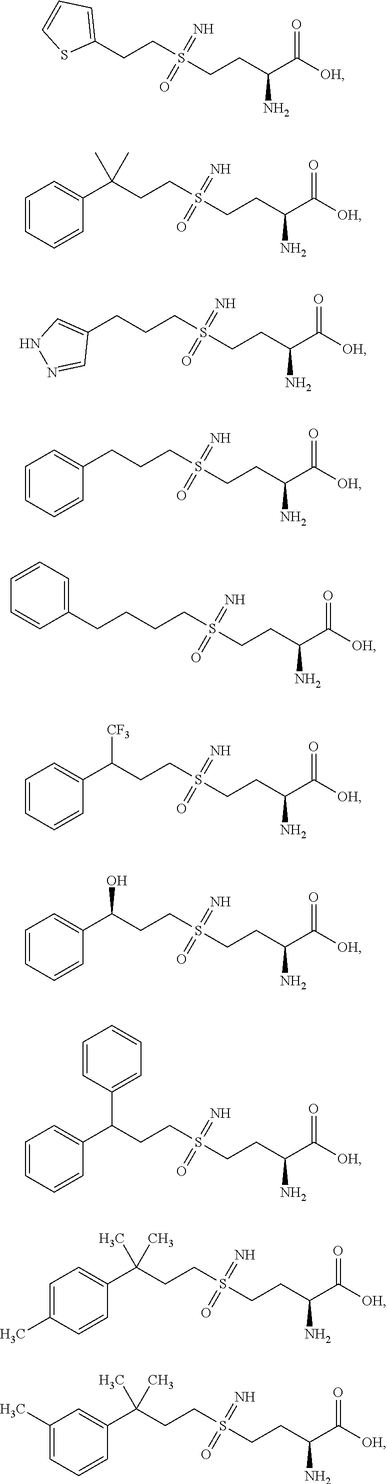

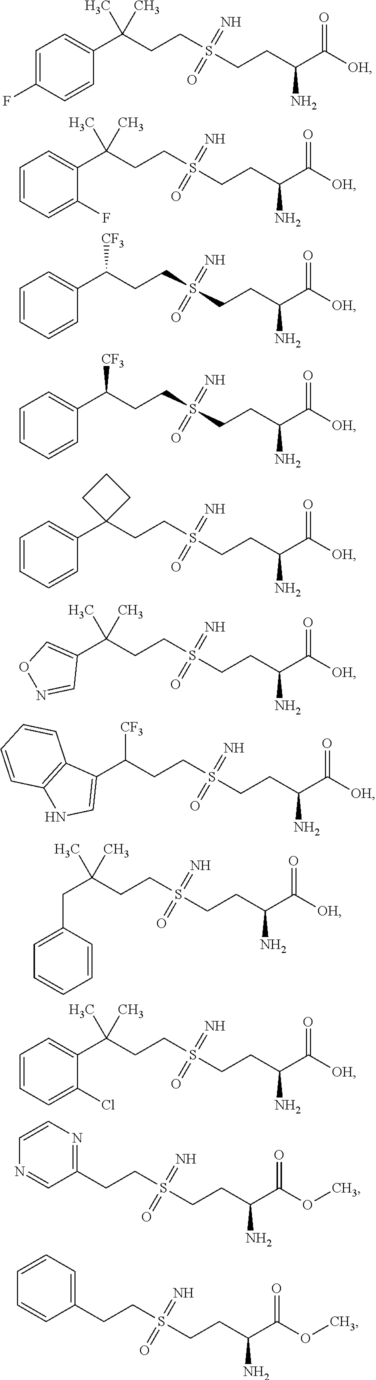

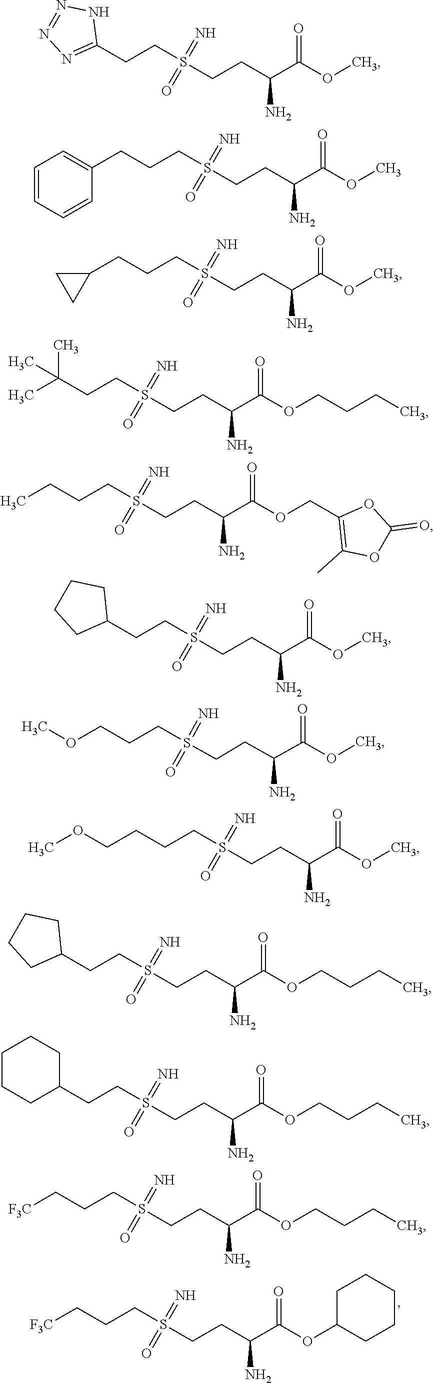

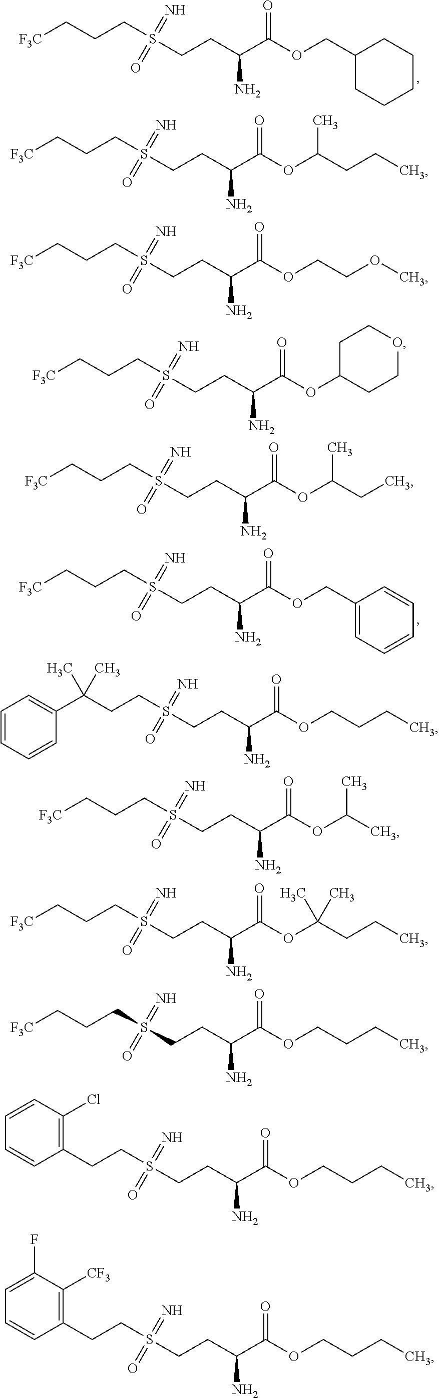

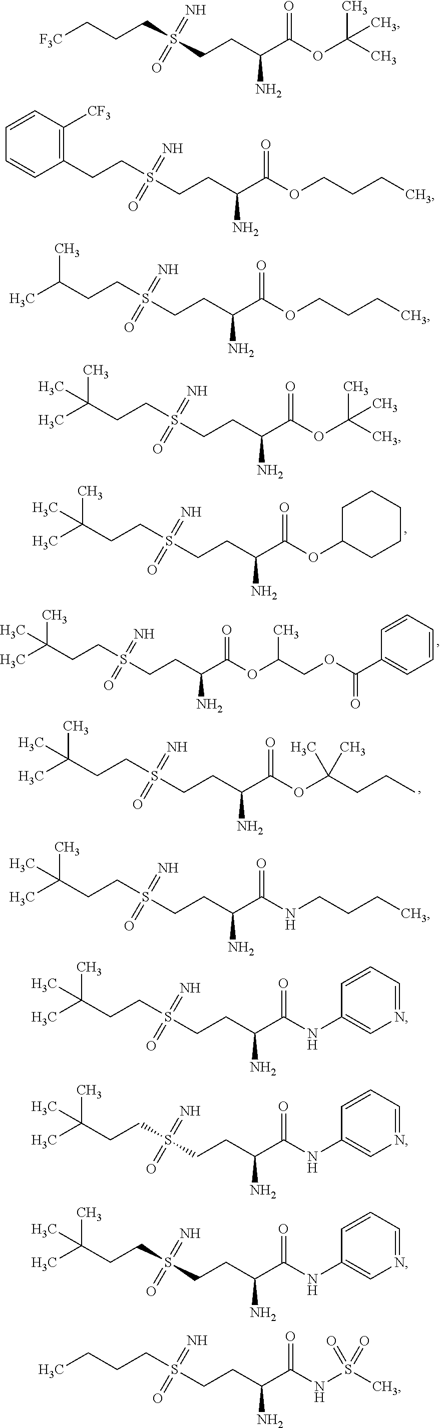

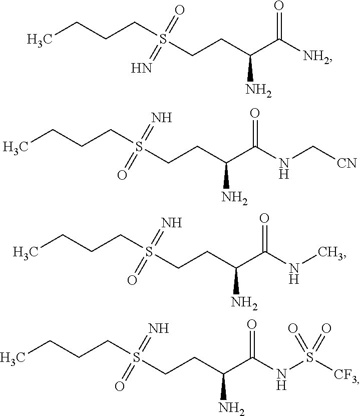

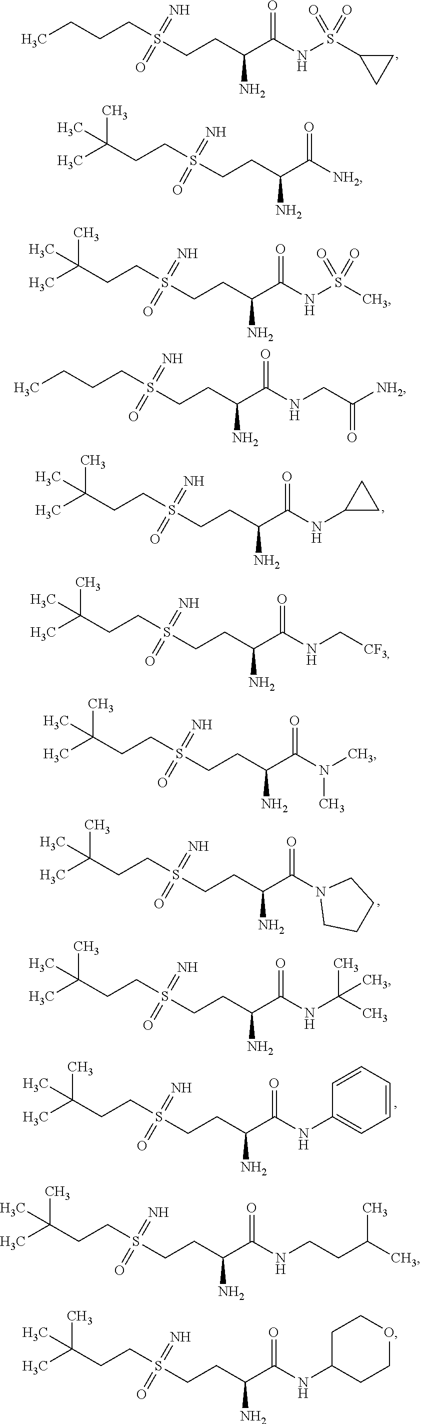

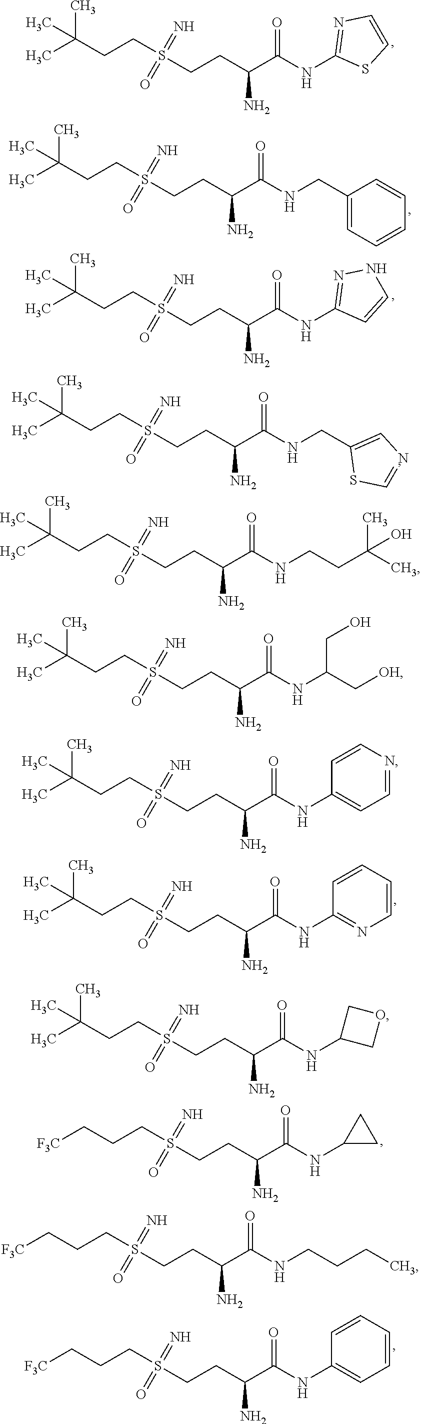

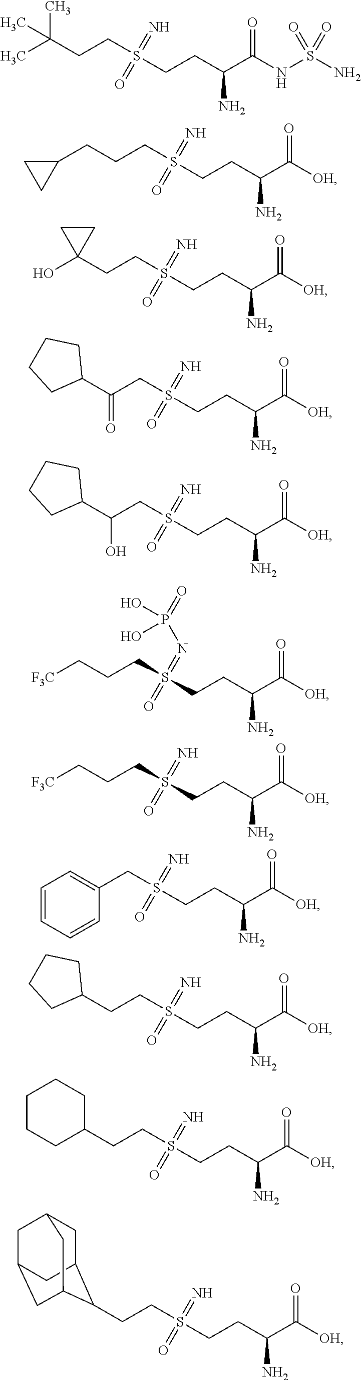

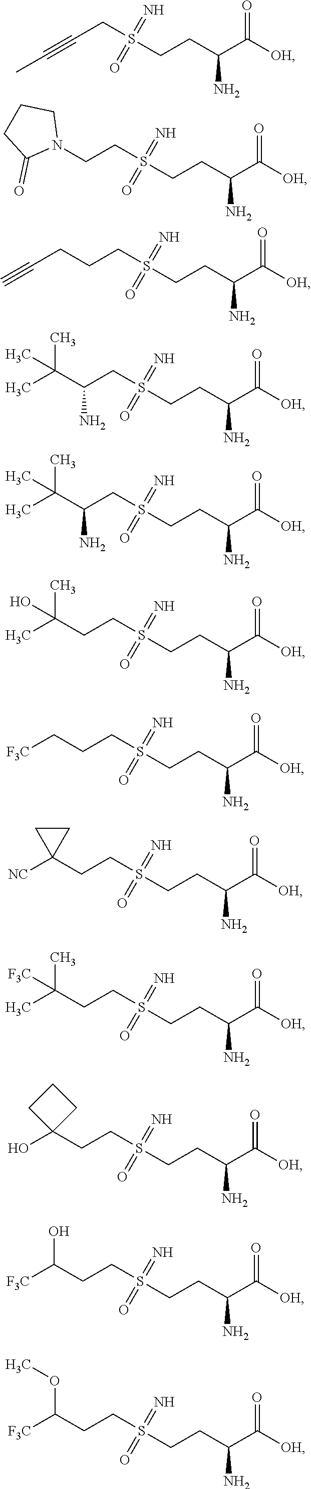

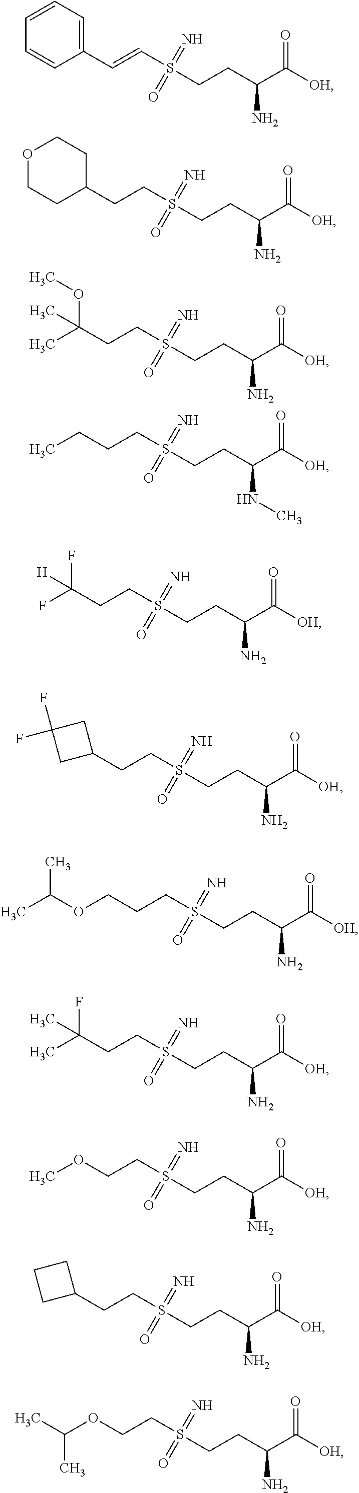

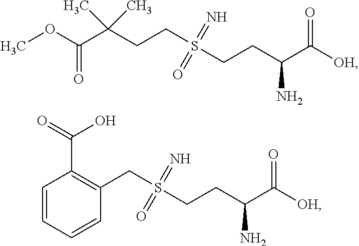 [SMILES: COC(=O)C(C)(C)CCS(=N)(=O)CC[C@H](N)C(=O)O ; N=S(=O)(CC[C@H](N)C(=O)O)Cc1ccccc1C(=O)O]

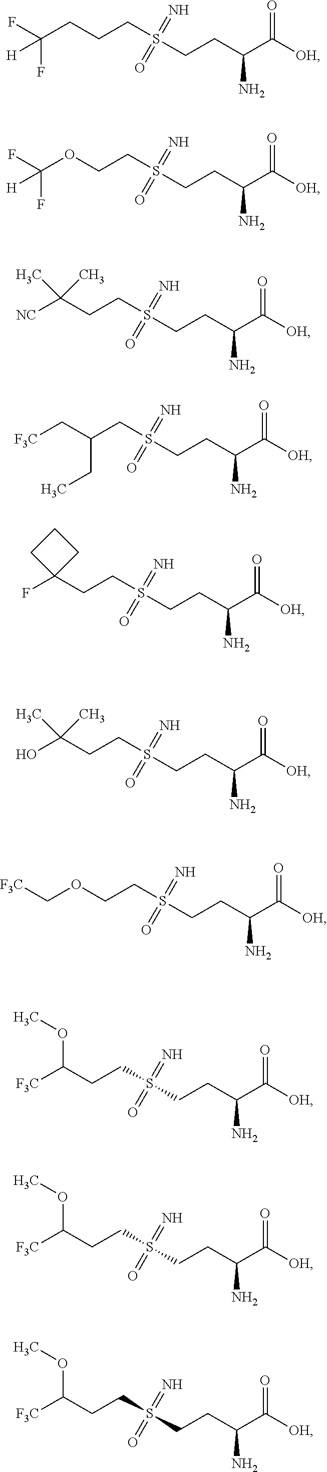

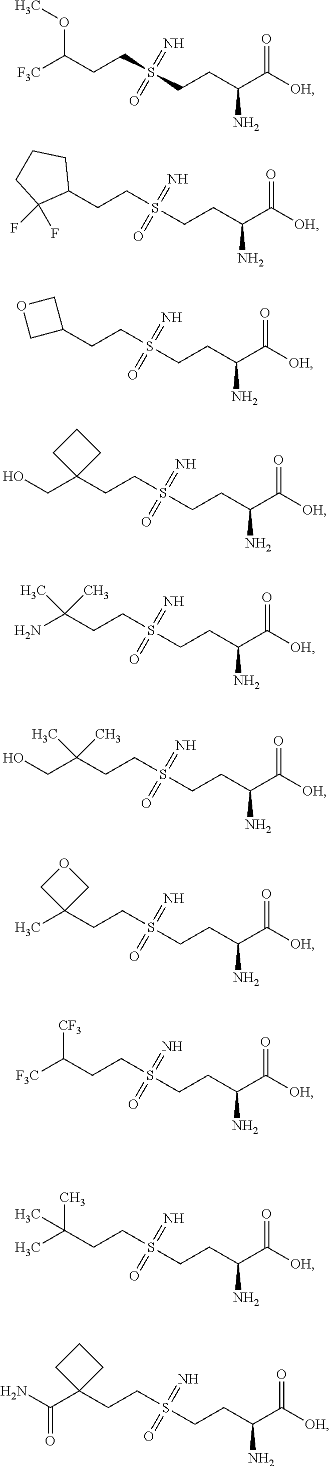

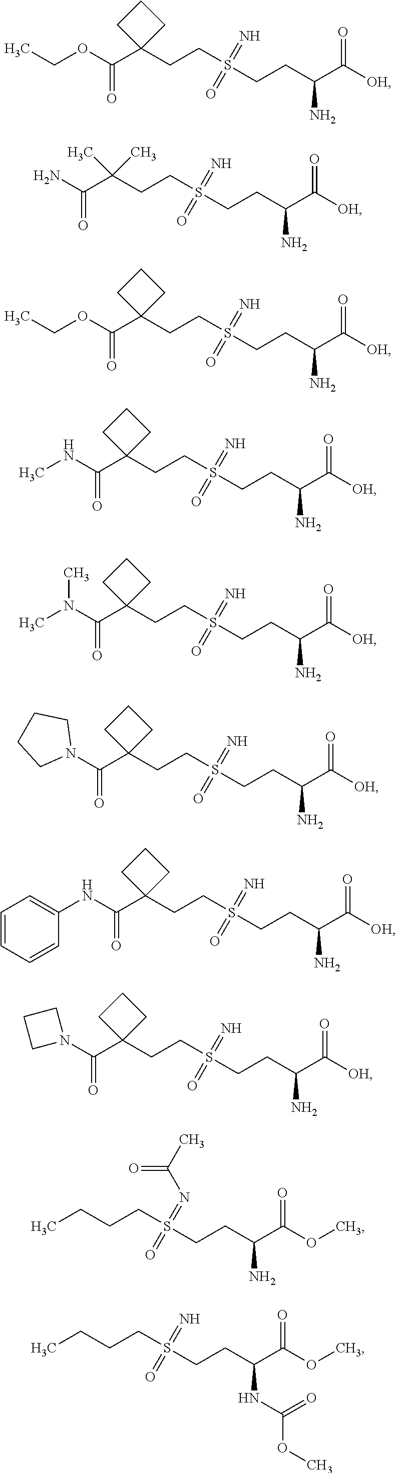

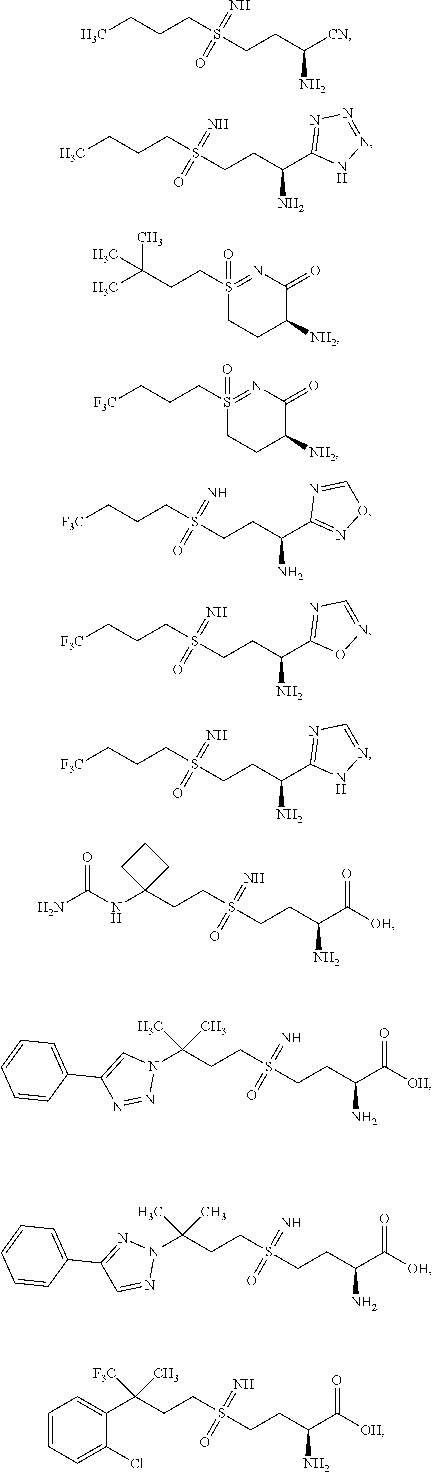

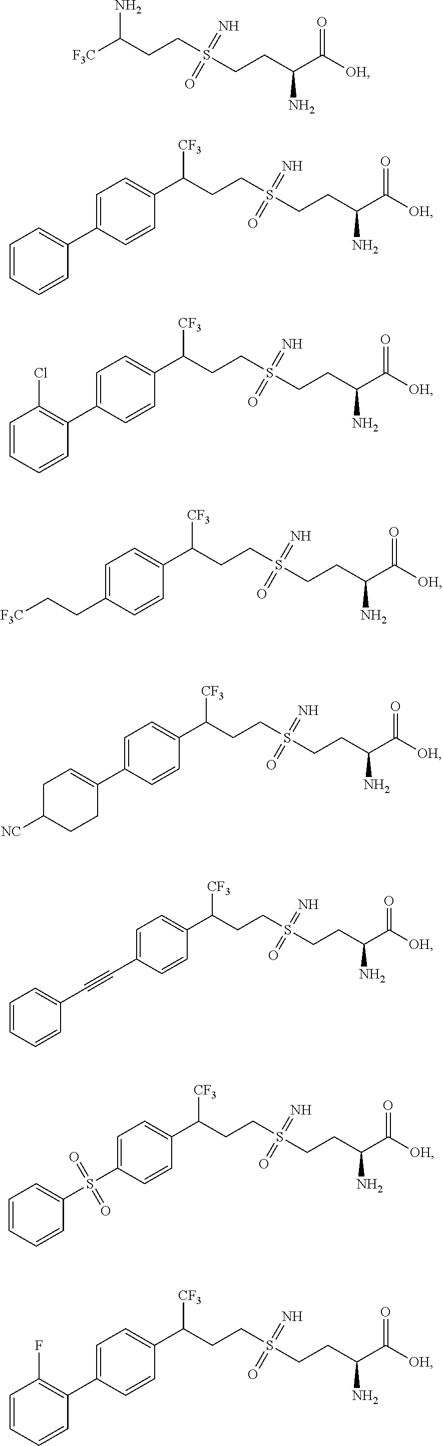

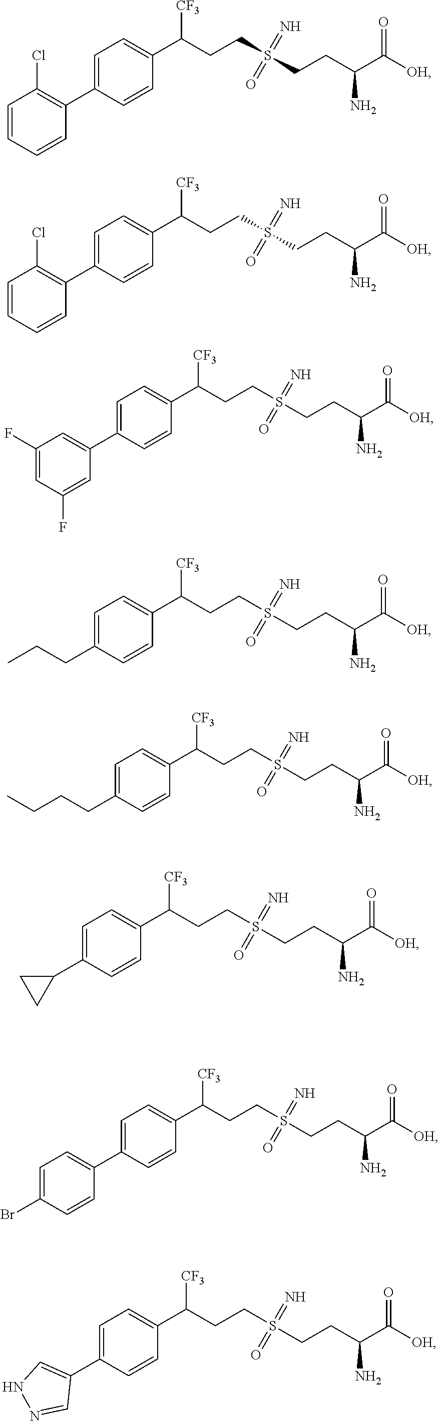

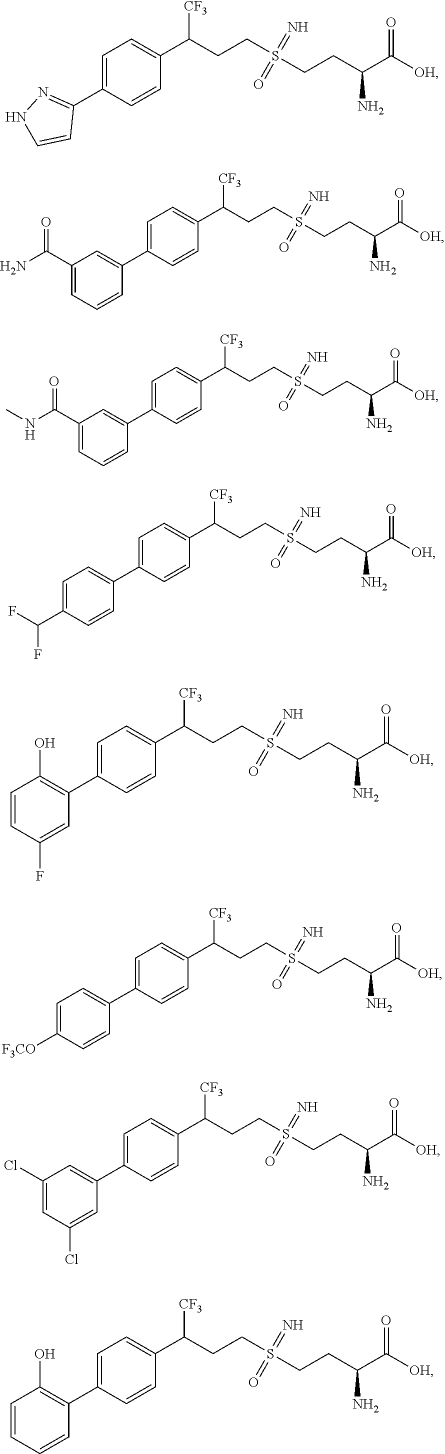

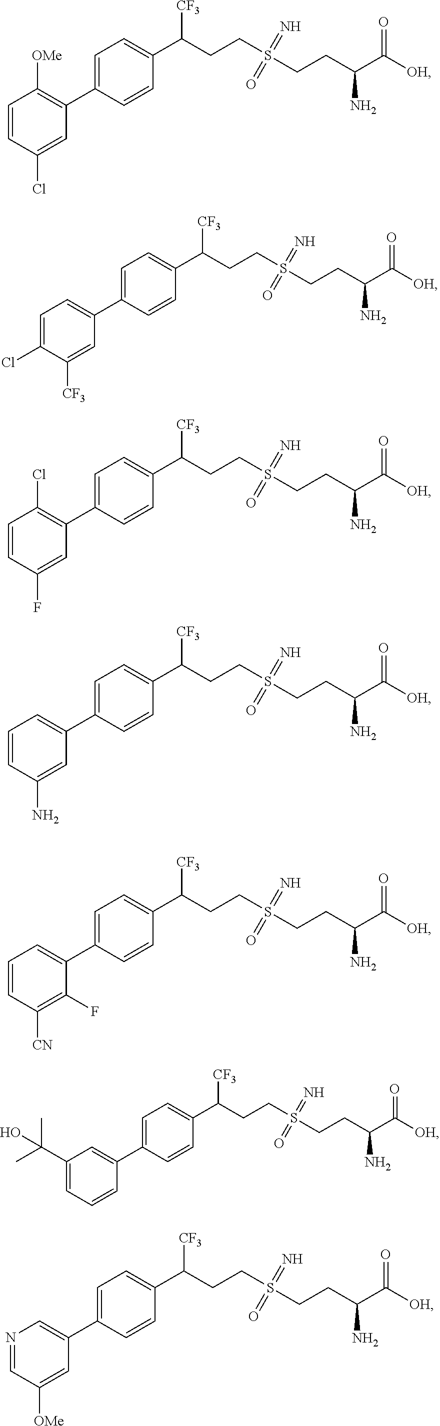

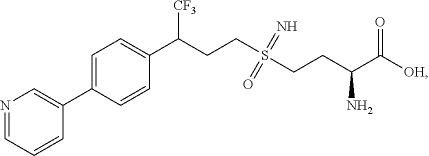 [SMILES: N=S(=O)(CCC(c1ccc(-c2cccnc2)cc1)C(F)(F)F)CC[C@H](N)C(=O)O]

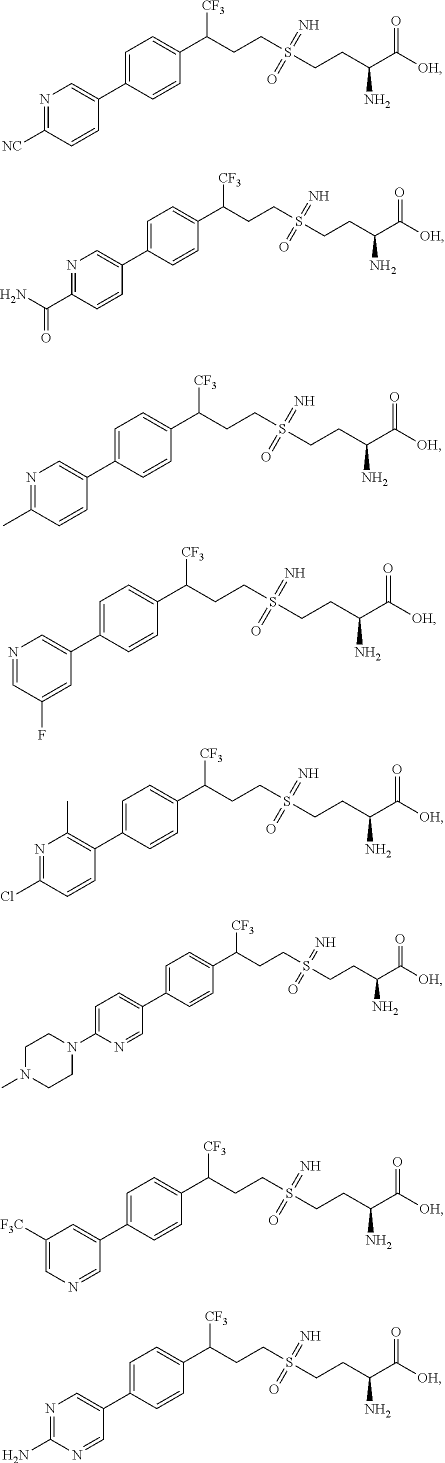

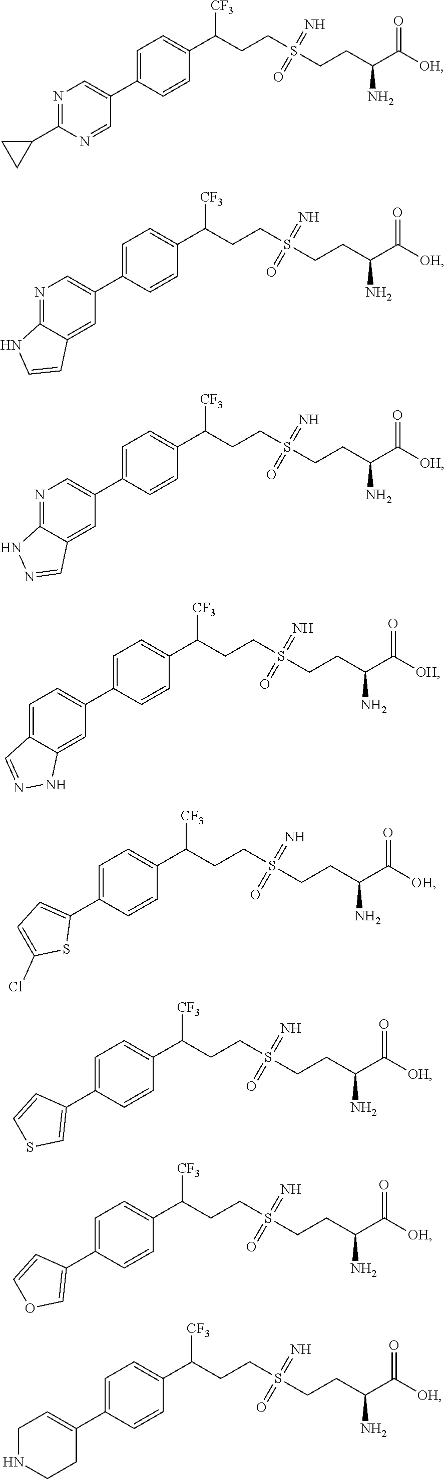

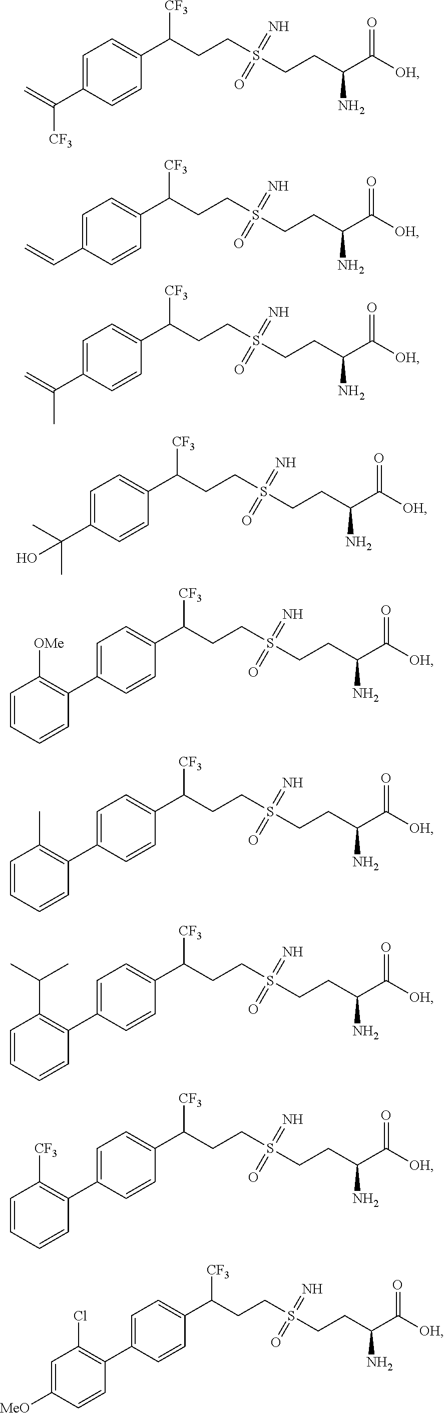

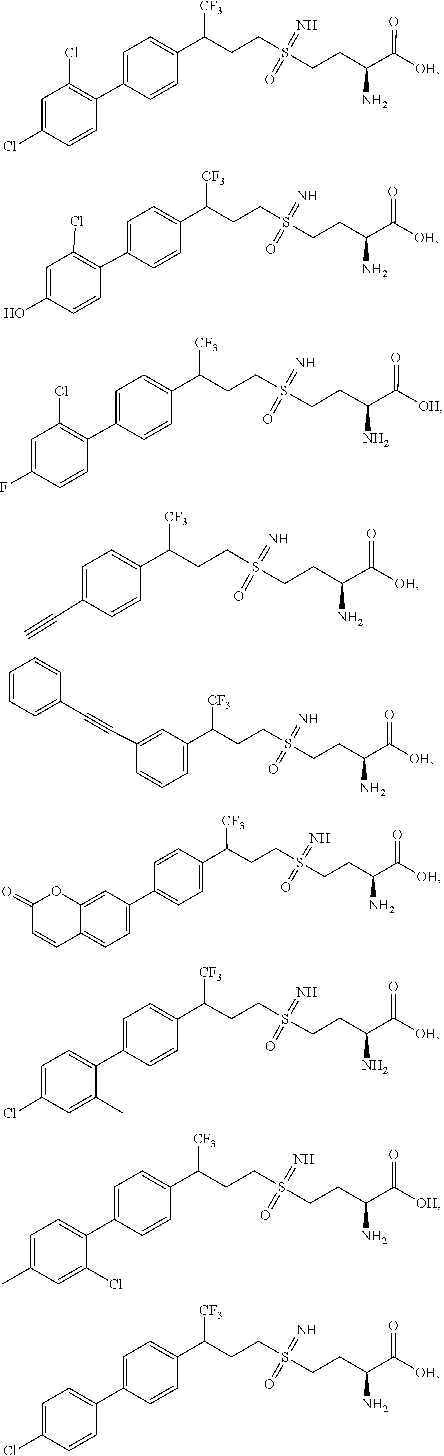

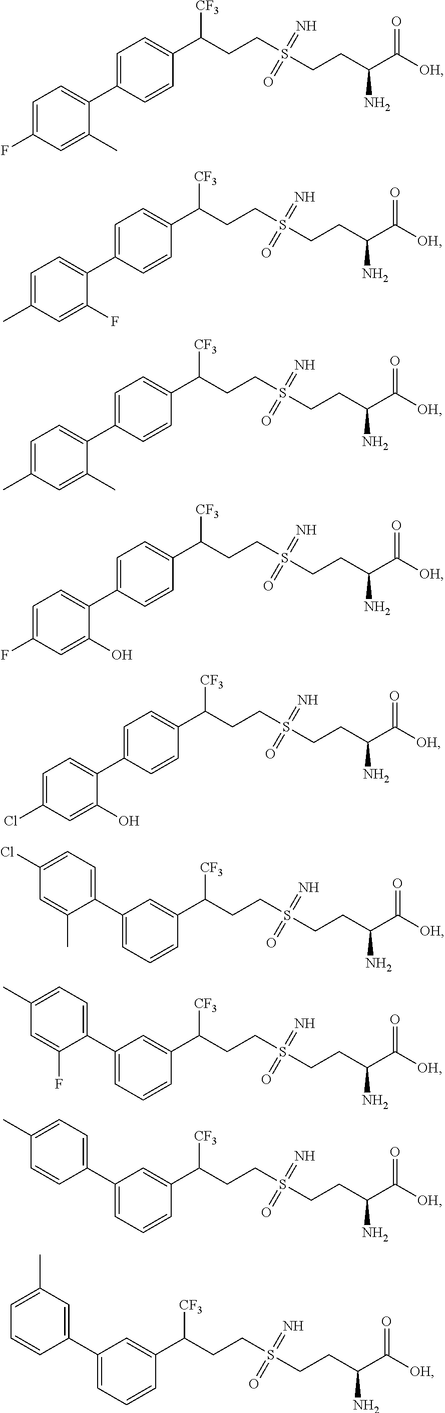

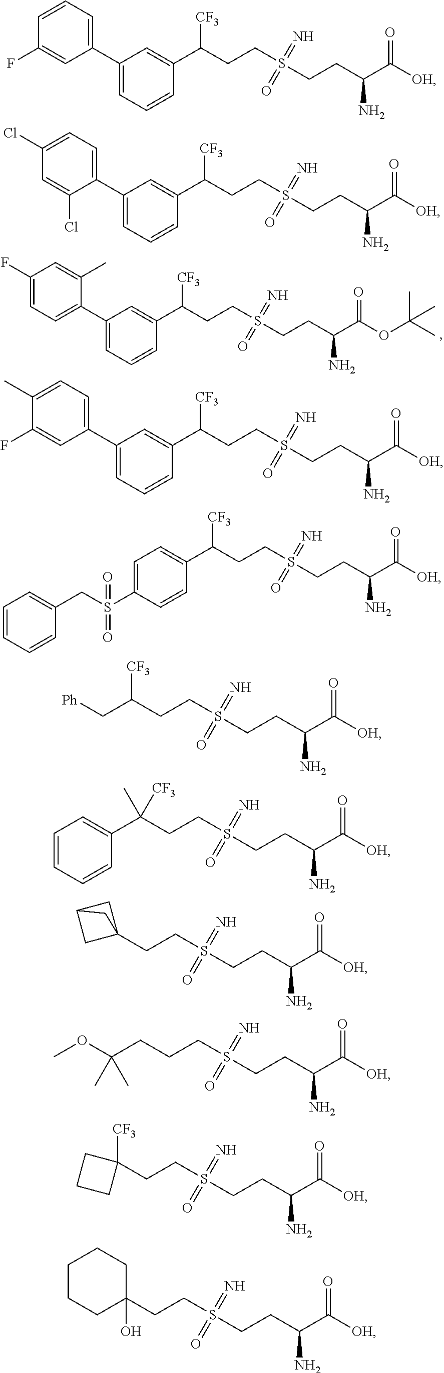

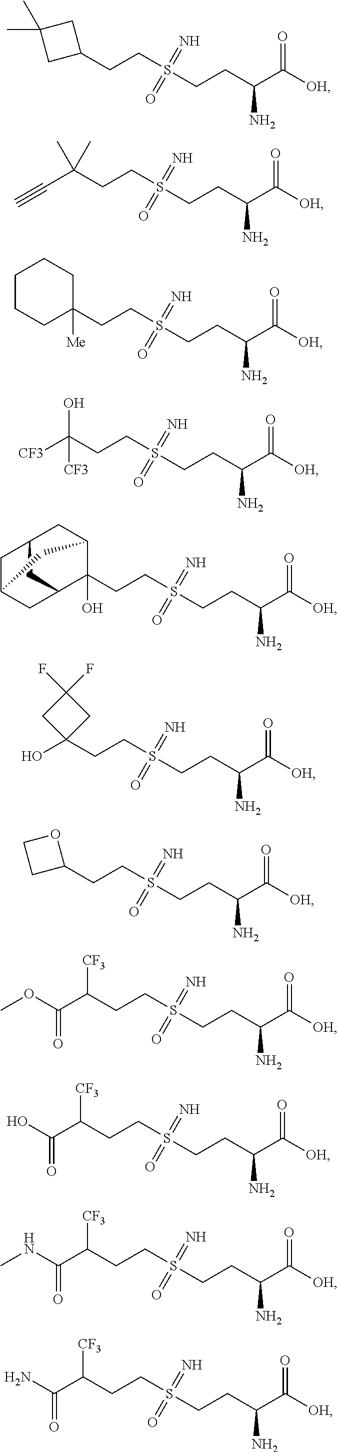

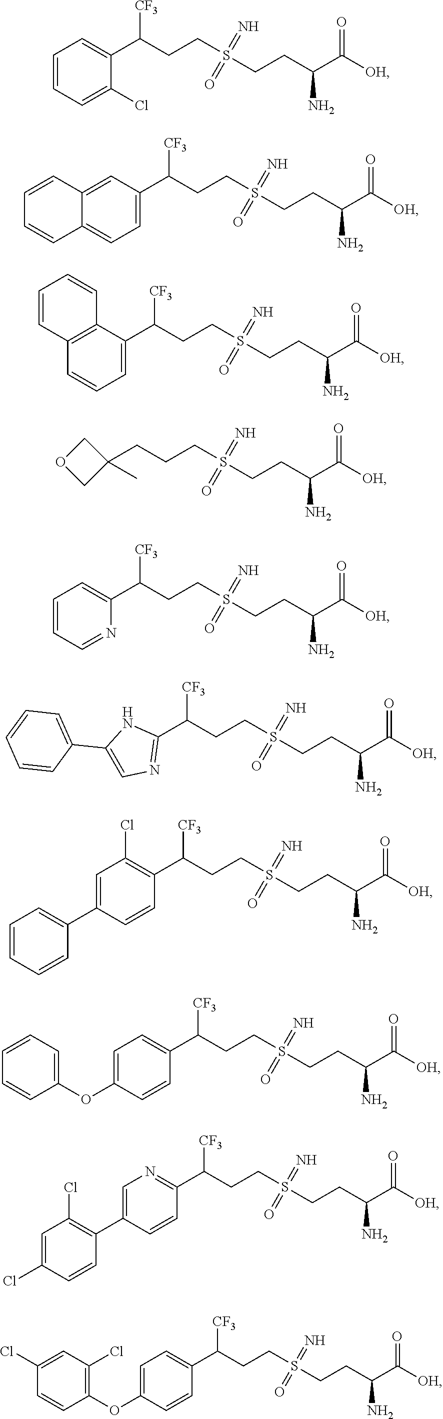

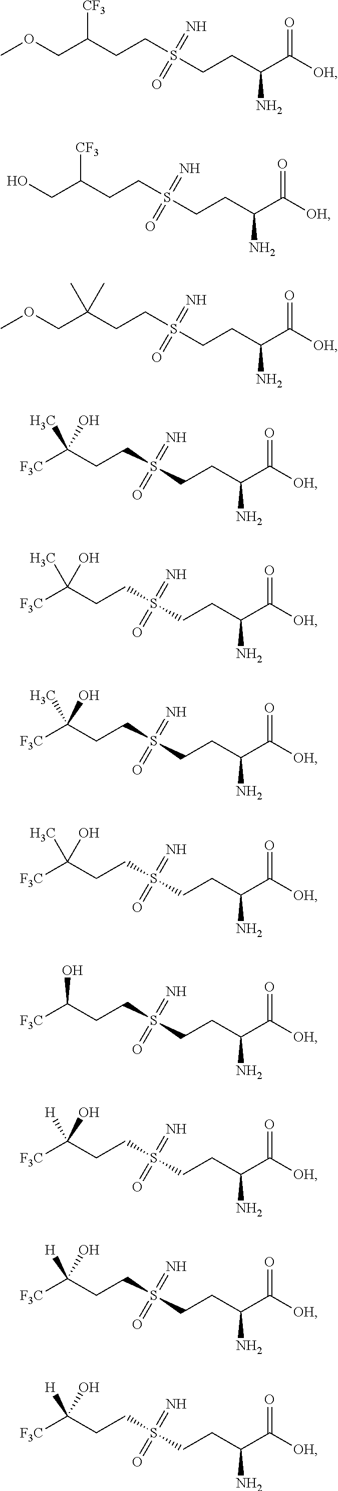

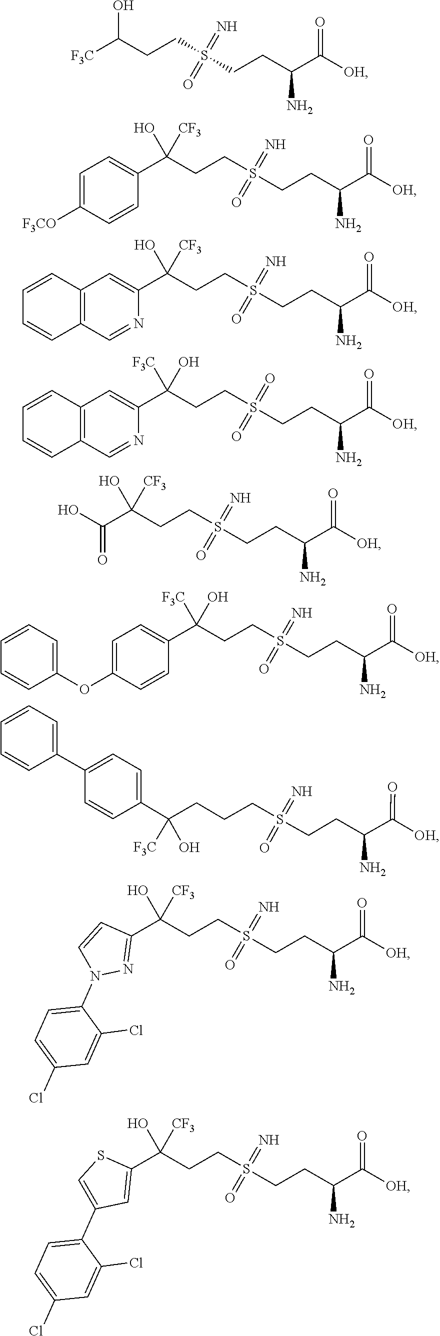

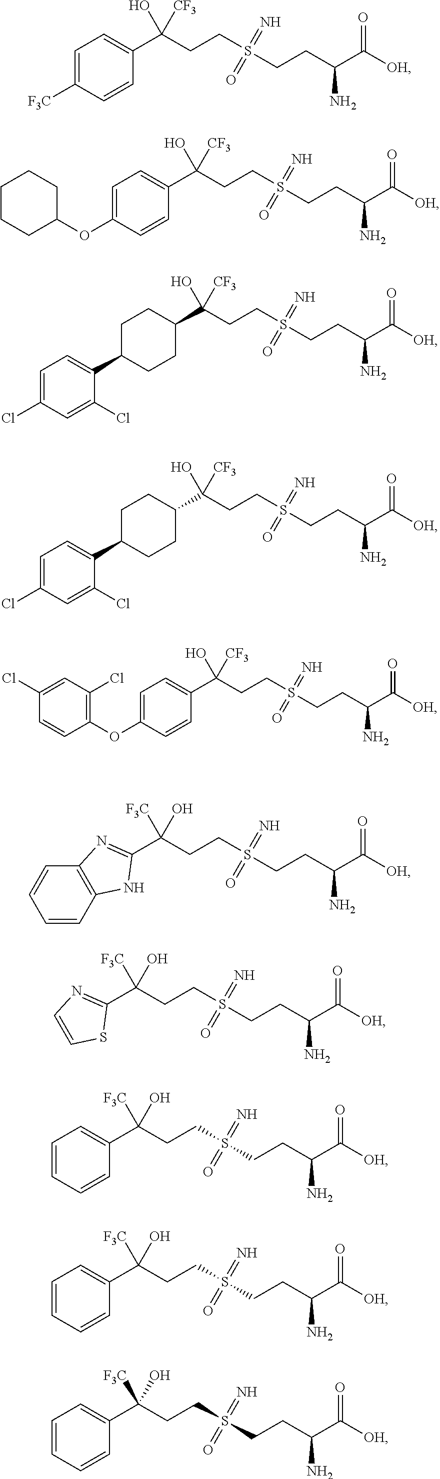

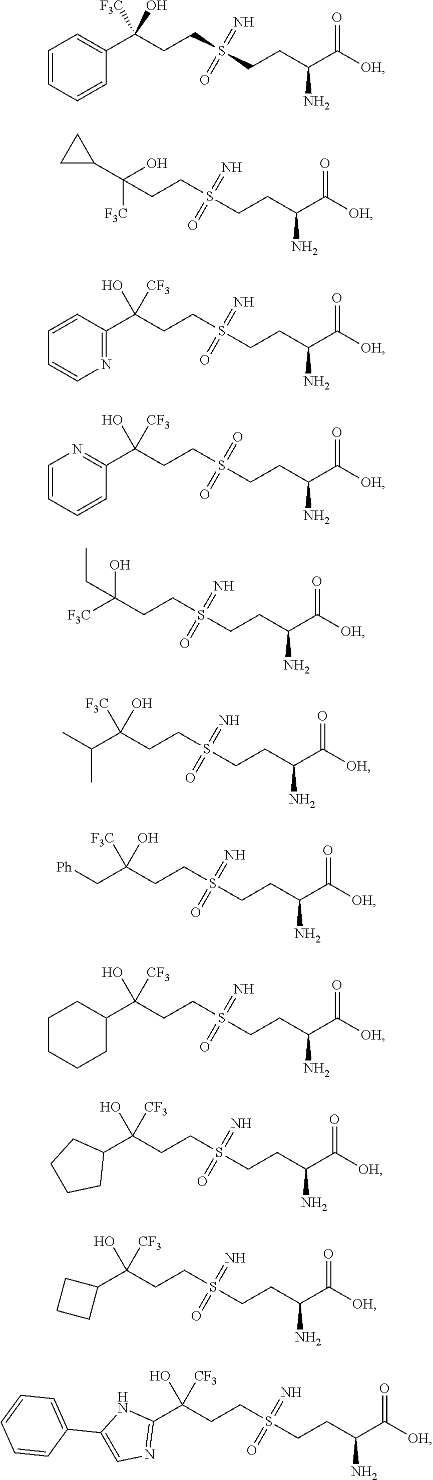

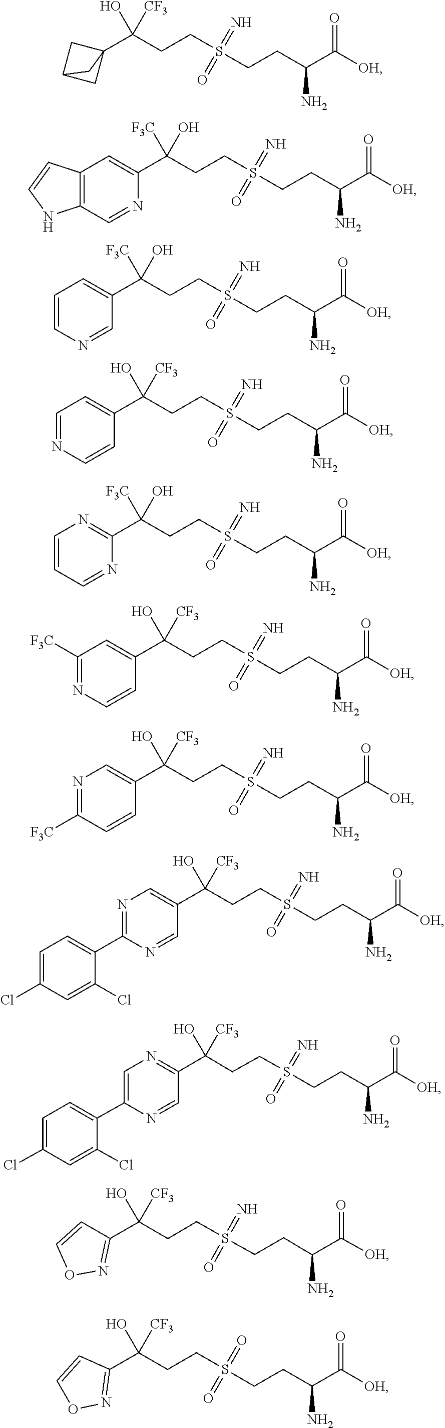

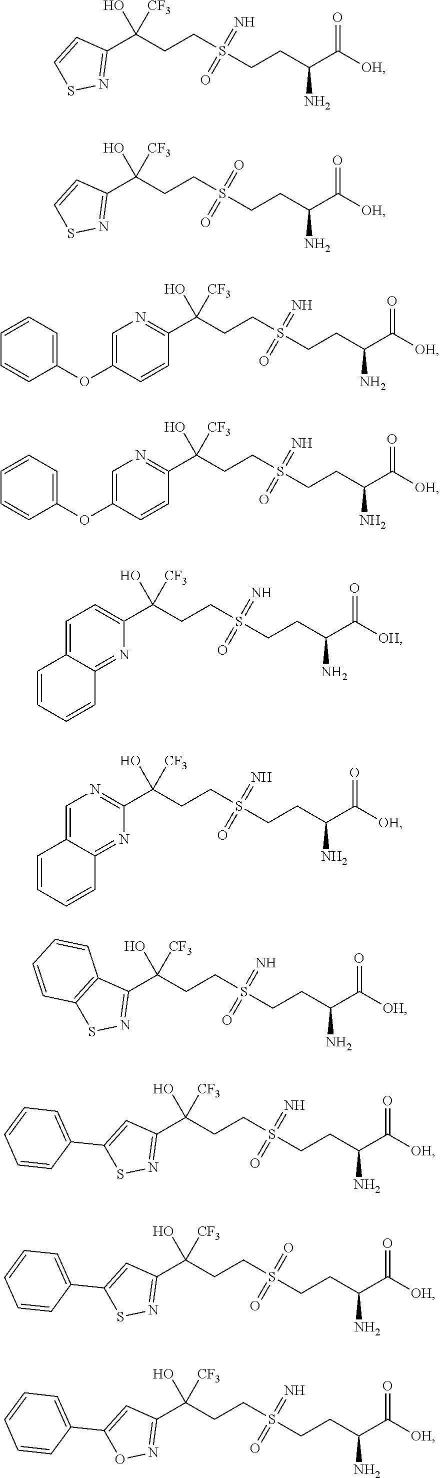

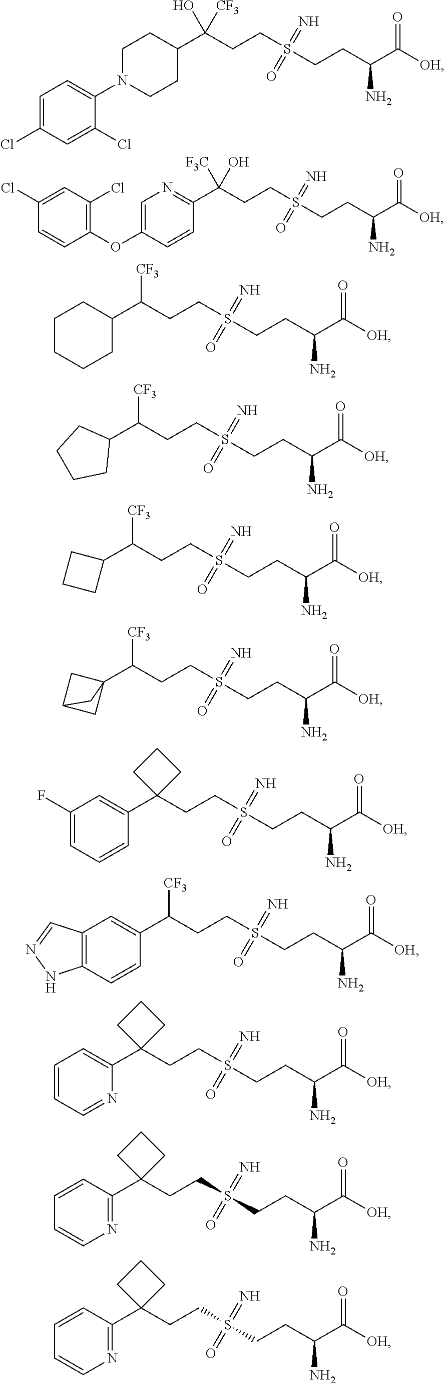

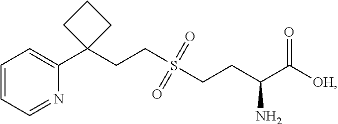 [SMILES: N[C@@H](CCS(=O)(=O)CCC1(c2ccccn2)CCC1)C(=O)O]

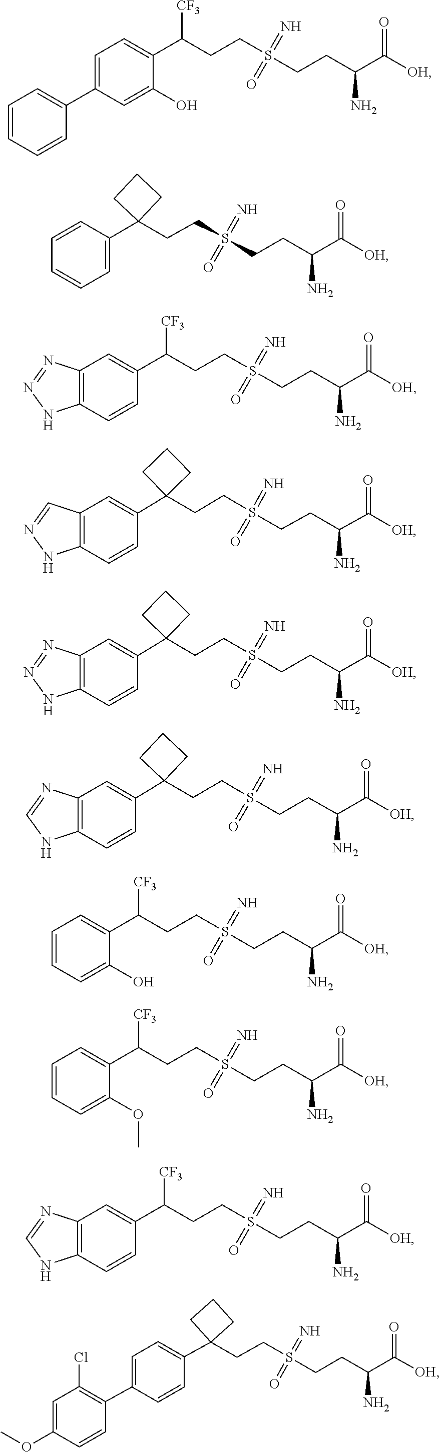

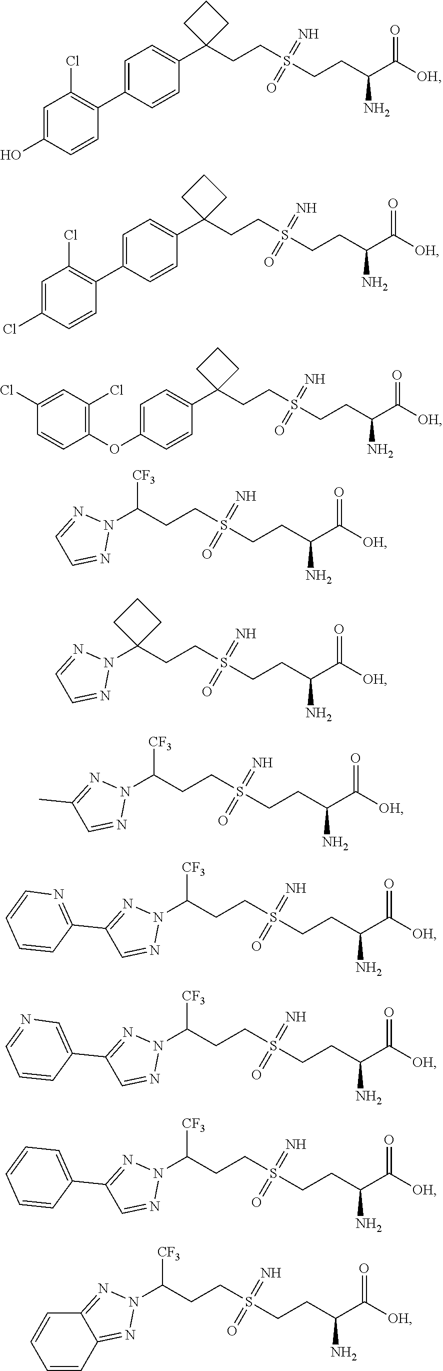

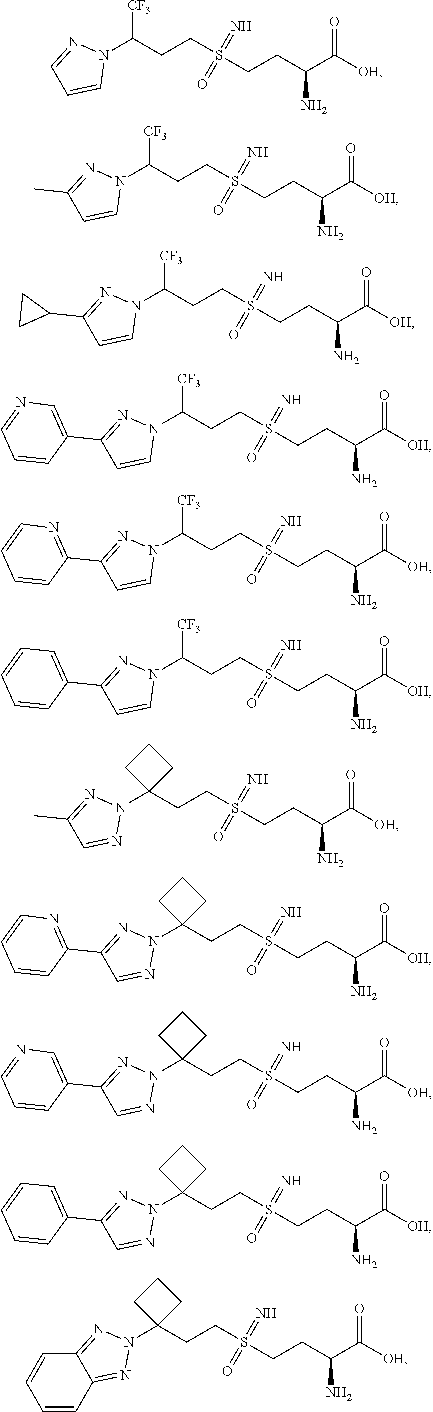

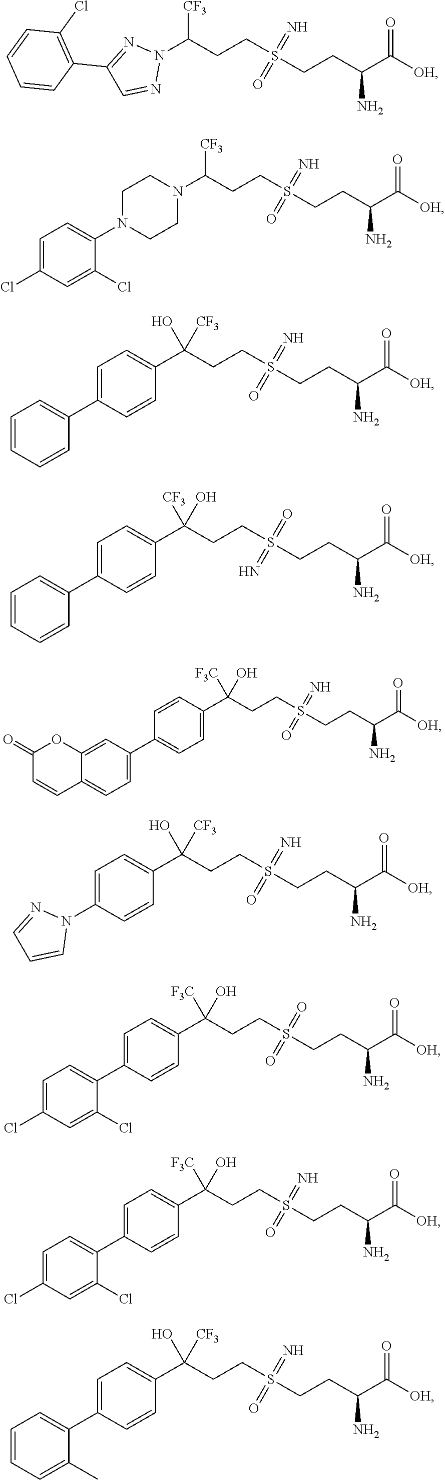

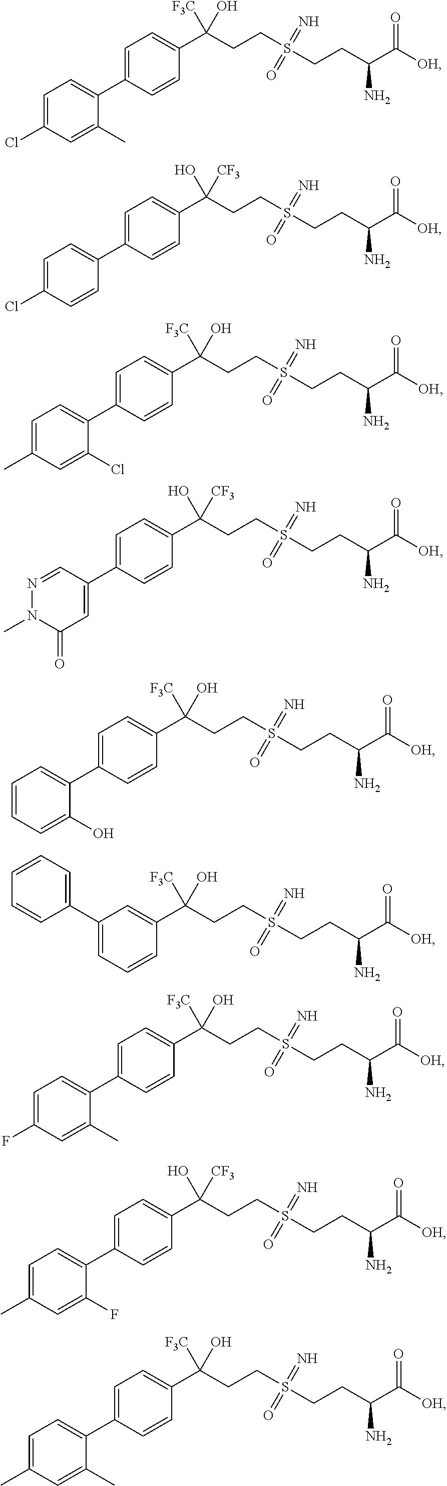

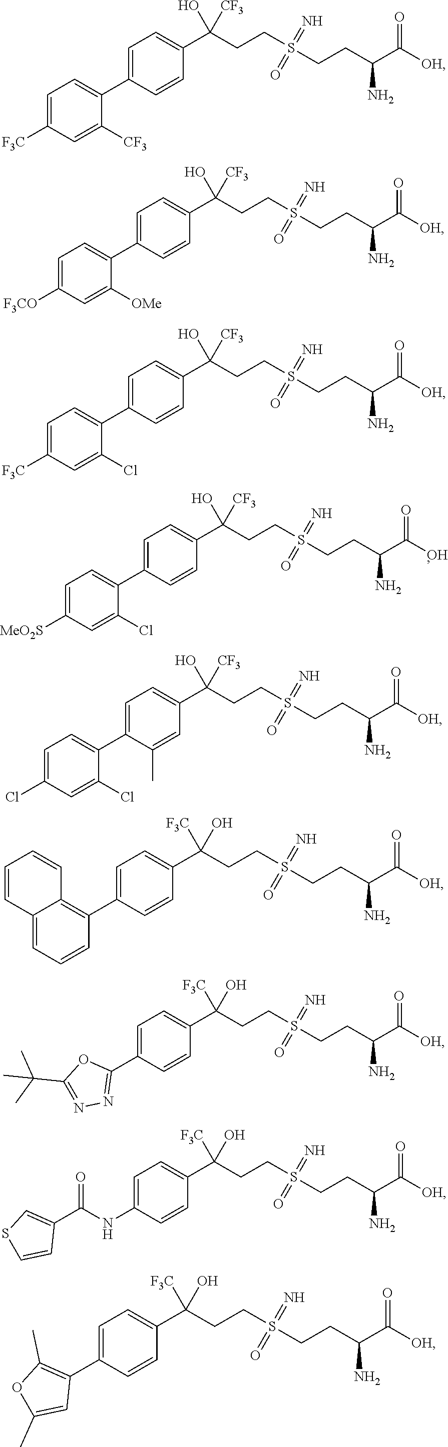

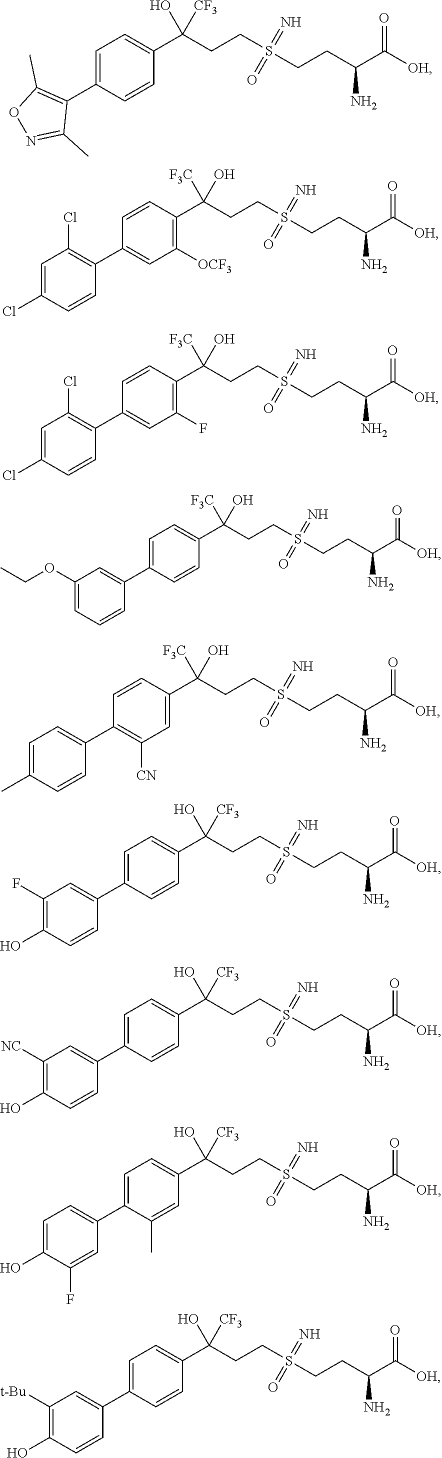

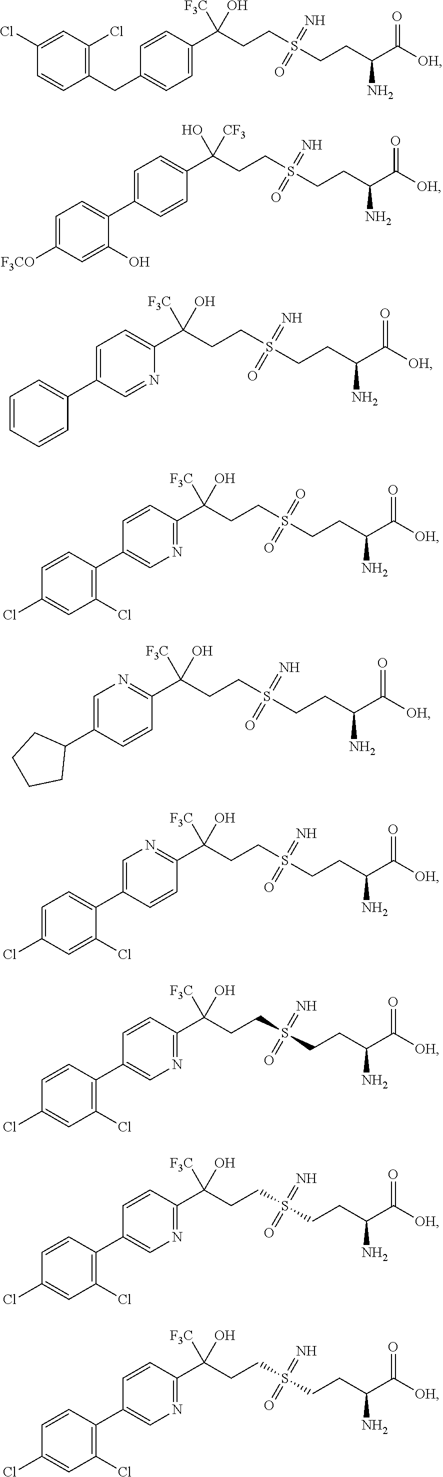

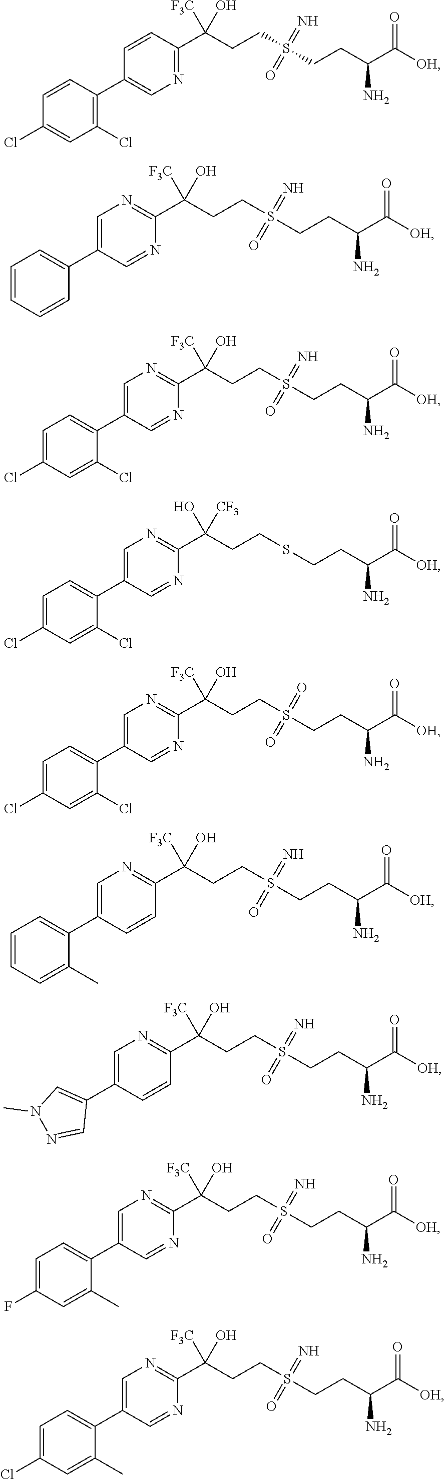

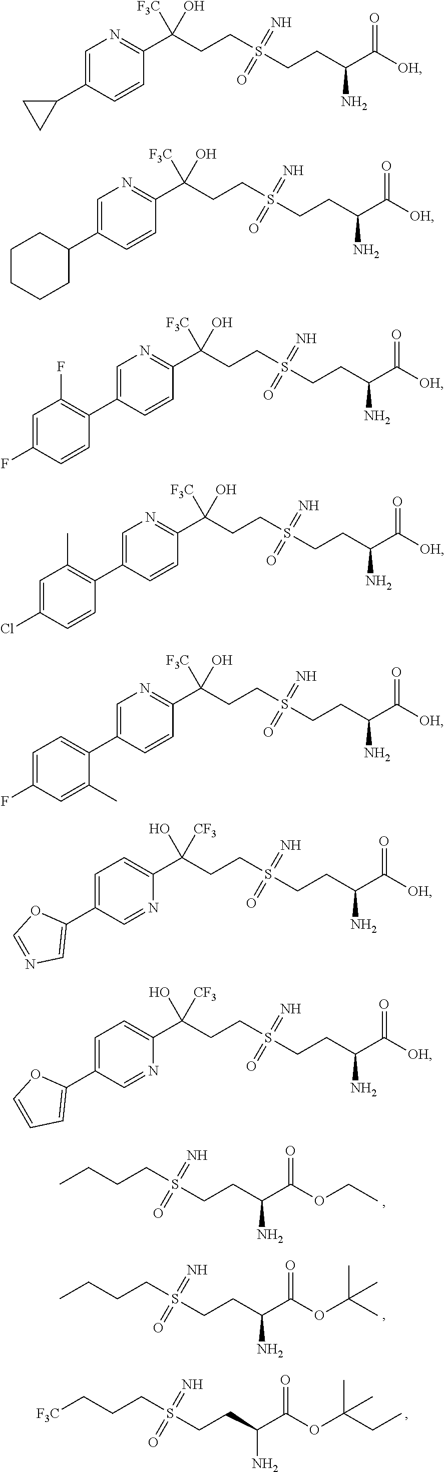

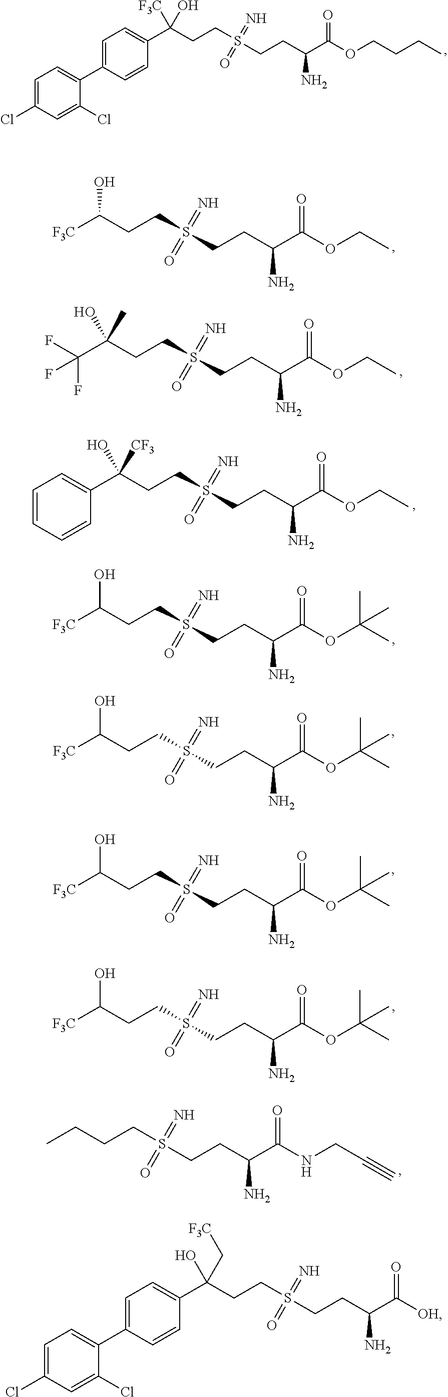

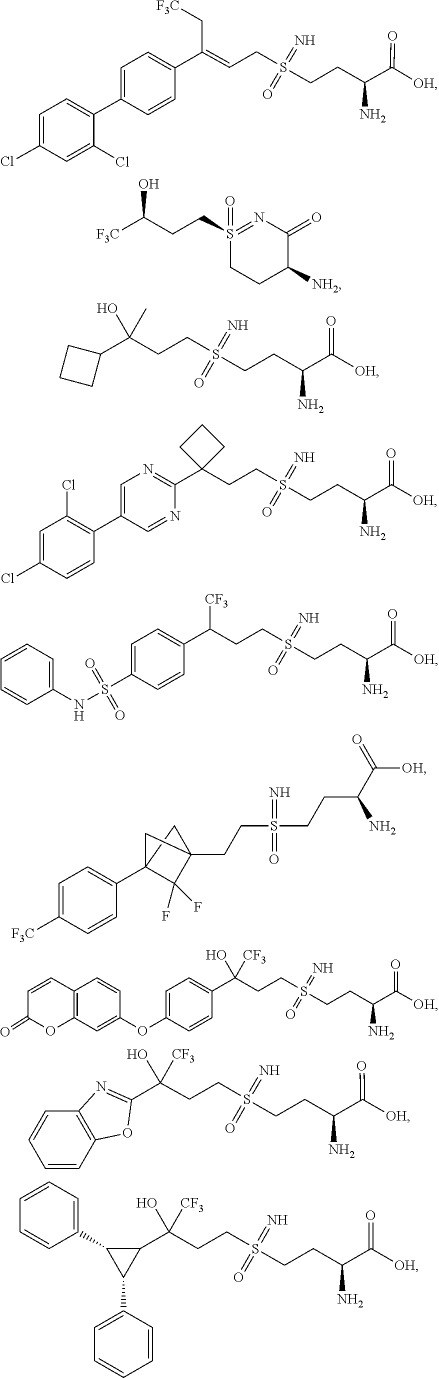

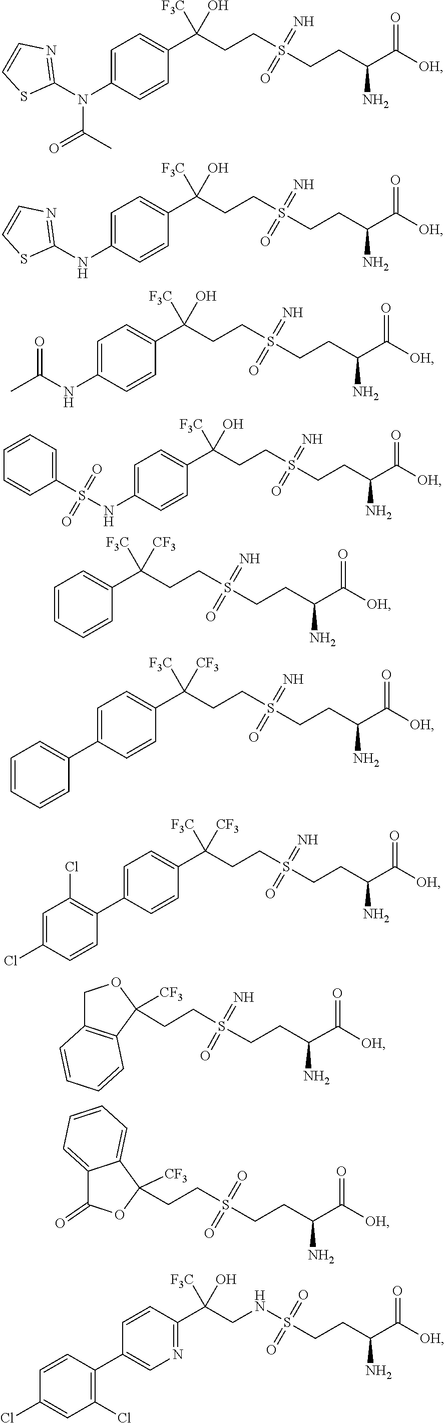

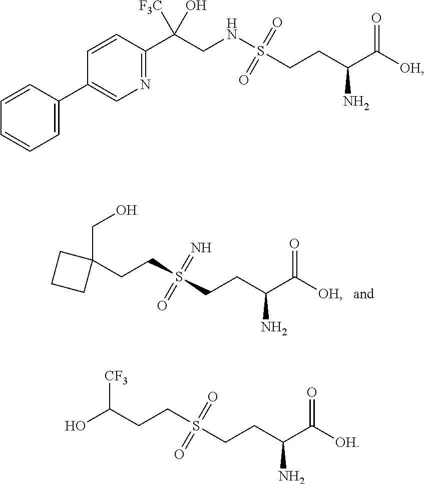 [SMILES: N[C@@H](CCS(=O)(=O)NCC(O)(c1ccc(-c2ccccc2)cn1)C(F)(F)F)C(=O)O ; N=[S@@](=O)(CC[C@H](N)C(=O)O)CCC1(CO)CCC1 ; N[C@@H](CCS(=O)(=O)CCC(O)C(F)(F)F)C(=O)O]

In some embodiments is a compound, a diastereomer or an enantiomer of the compound, or a pharmaceutically acceptable salt of any of the foregoing, or a deuterated derivative of any of the foregoing; wherein the compound is selected from the group consisting of:

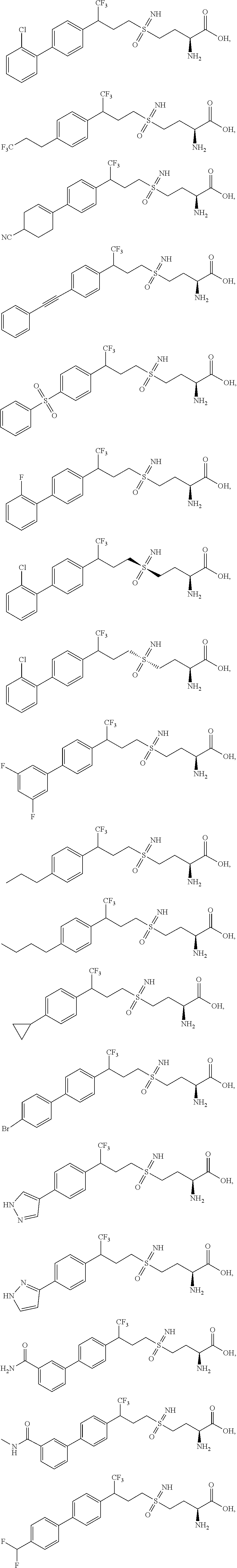

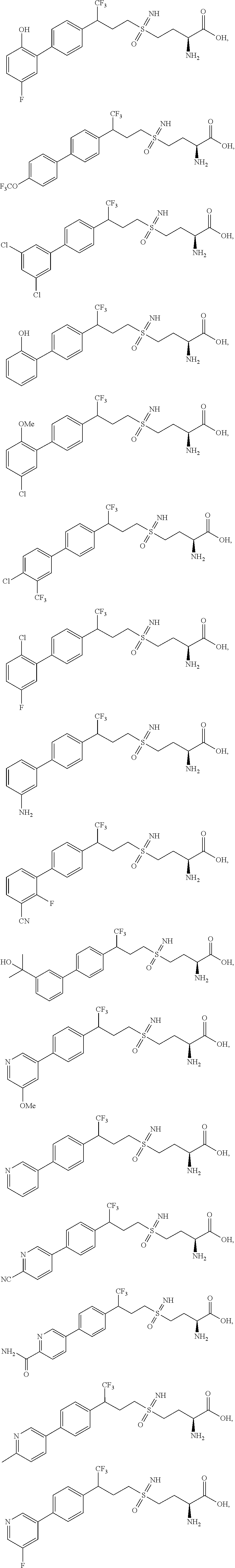

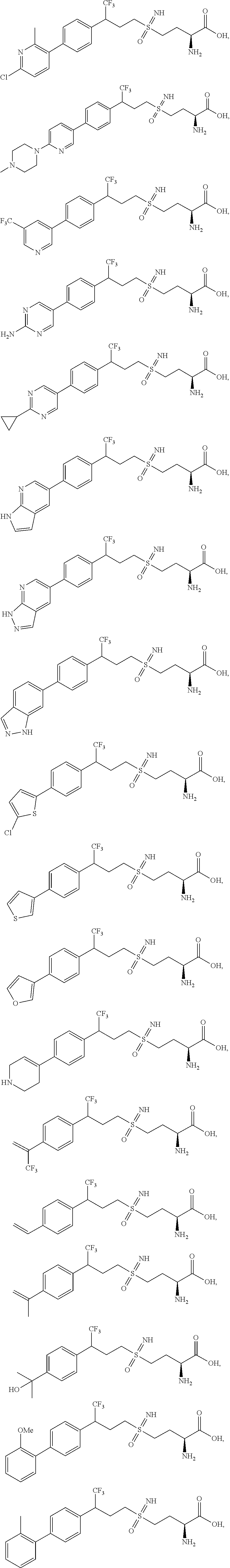

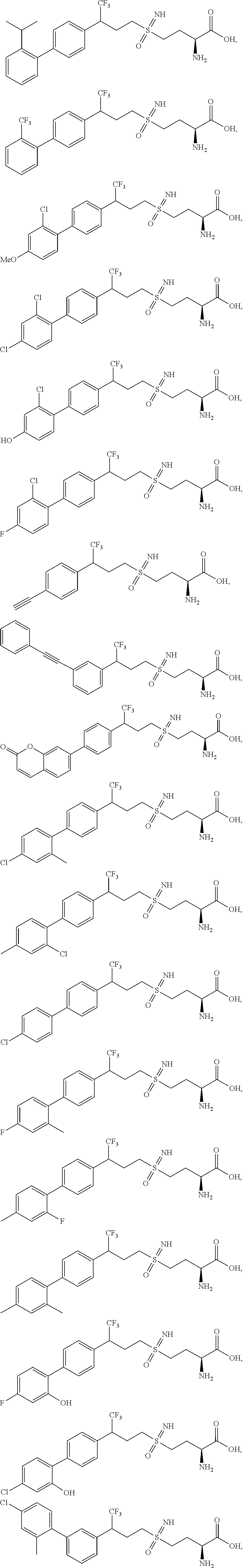

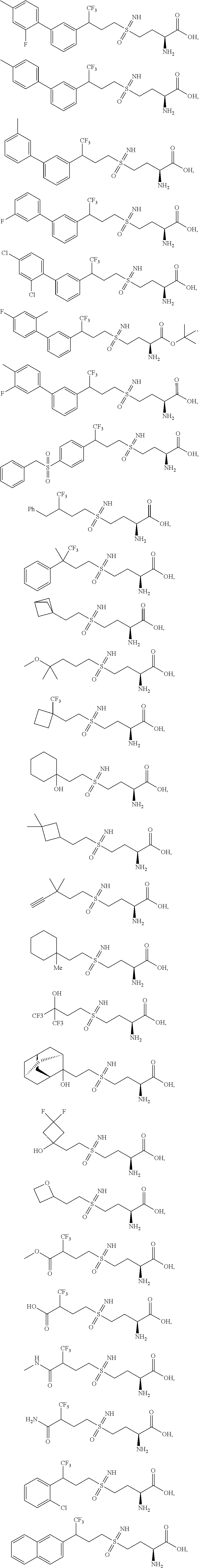

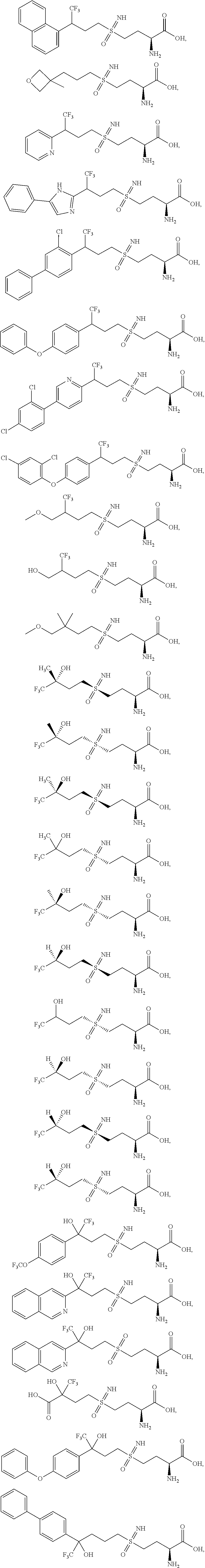

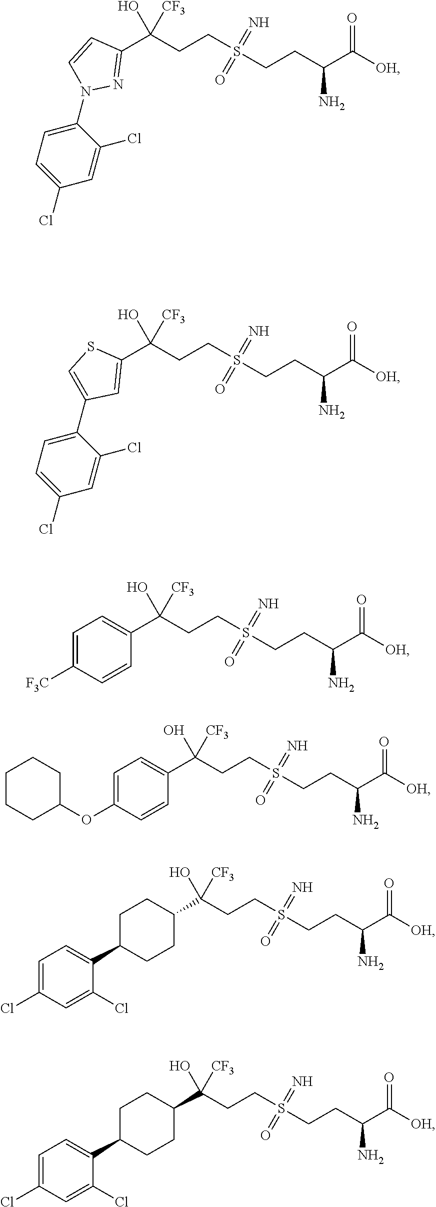 [SMILES: N=S(=O)(CC[C@H](N)C(=O)O)CCC(O)(c1ccn(-c2ccc(Cl)cc2Cl)n1)C(F)(F)F ; N=S(=O)(CC[C@H](N)C(=O)O)CCC(O)(c1cc(-c2ccc(Cl)cc2Cl)cs1)C(F)(F)F ; N=S(=O)(CC[C@H](N)C(=O)O)CCC(O)(c1ccc(C(F)(F)F)cc1)C(F)(F)F ; N=S(=O)(CC[C@H](N)C(=O)O)CCC(O)(c1ccc(OC2CCCCC2)cc1)C(F)(F)F ; N=S(=O)(CC[C@H](N)C(=O)O)CCC(O)([C@H]1CC[C@H](c2ccc(Cl)cc2Cl)CC1)C(F)(F)F ; N=S(=O)(CC[C@H](N)C(=O)O)CCC(O)([C@H]1CC[C@@H](c2ccc(Cl)cc2Cl)CC1)C(F)(F)F]

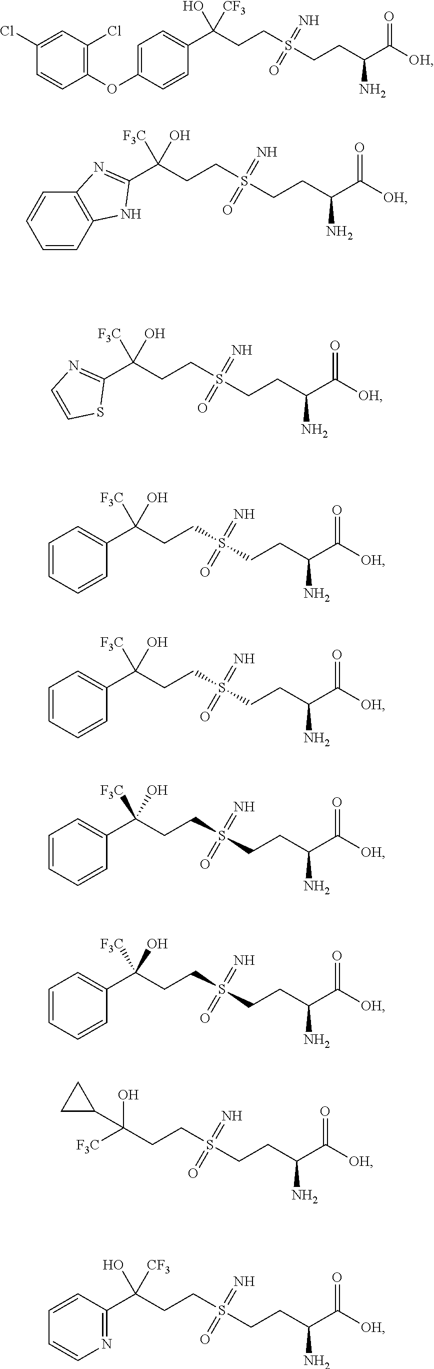

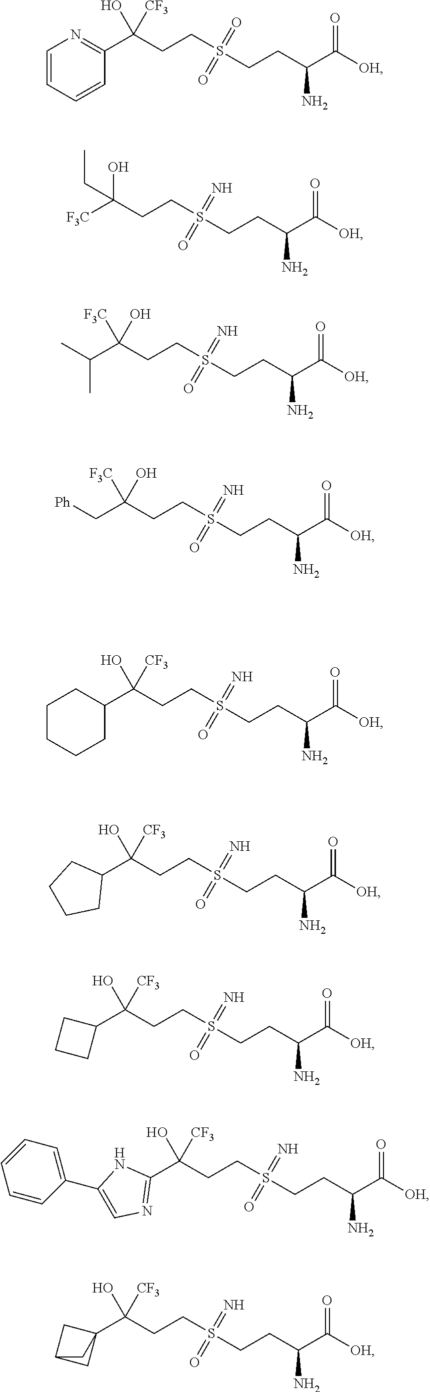

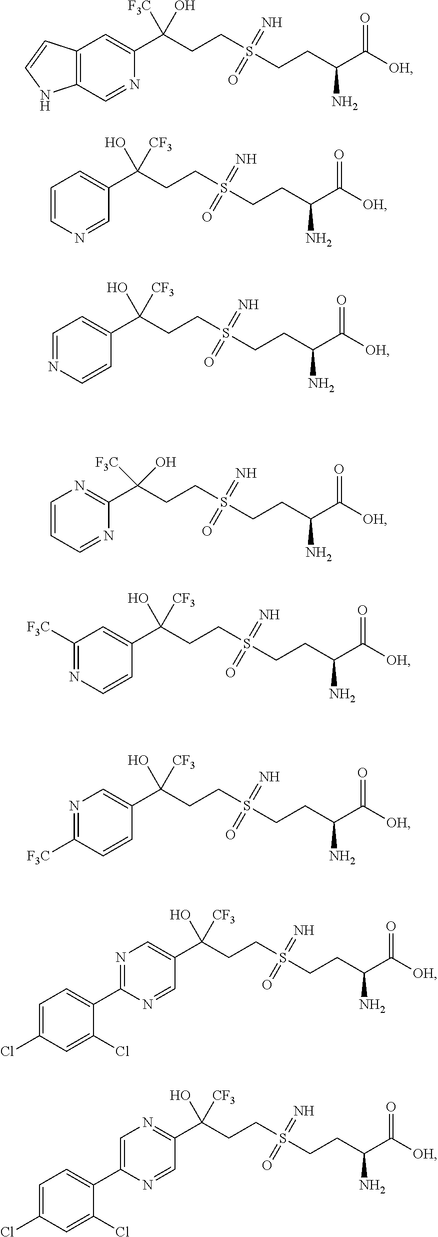

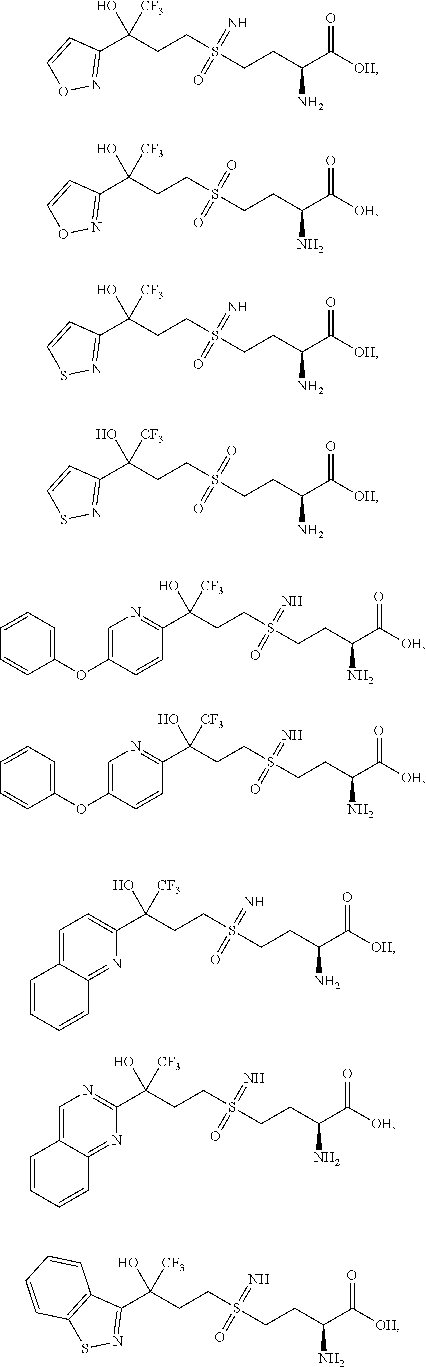

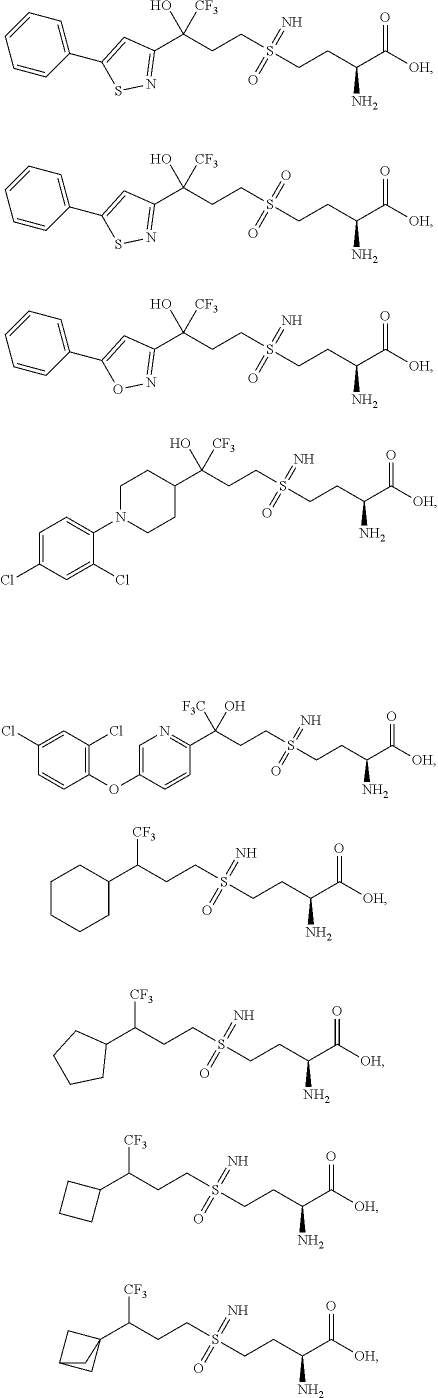

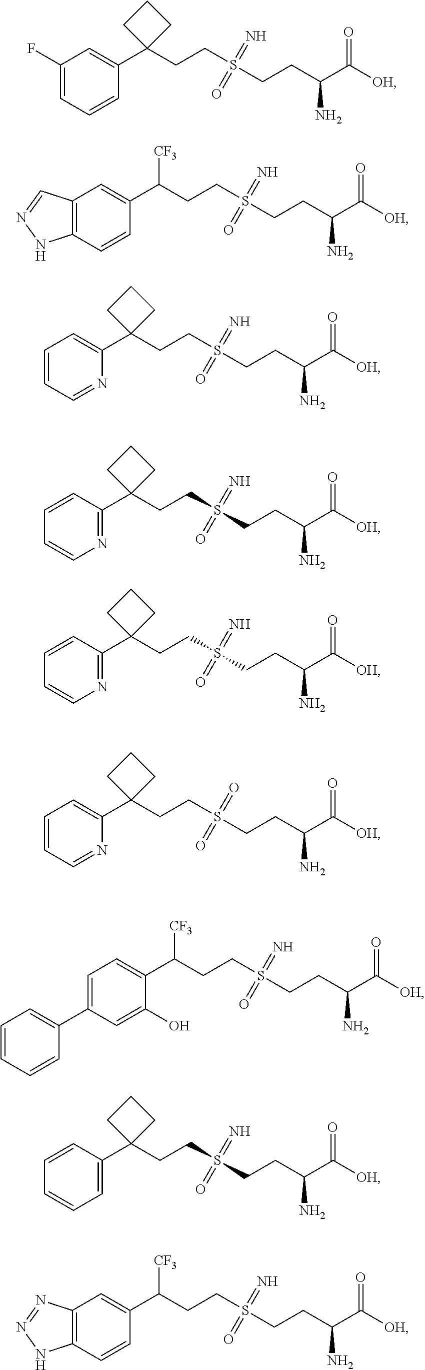

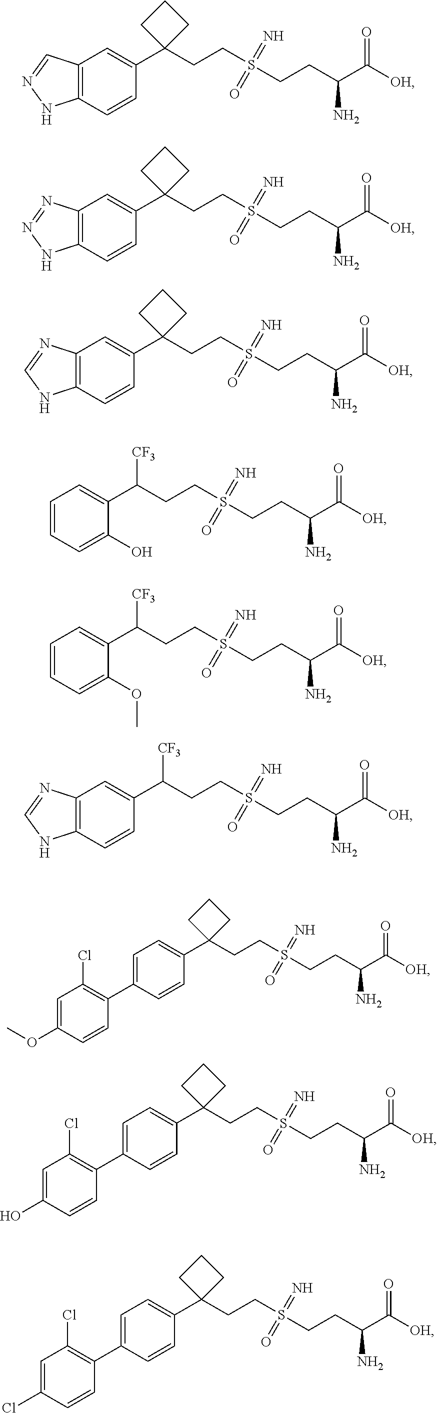

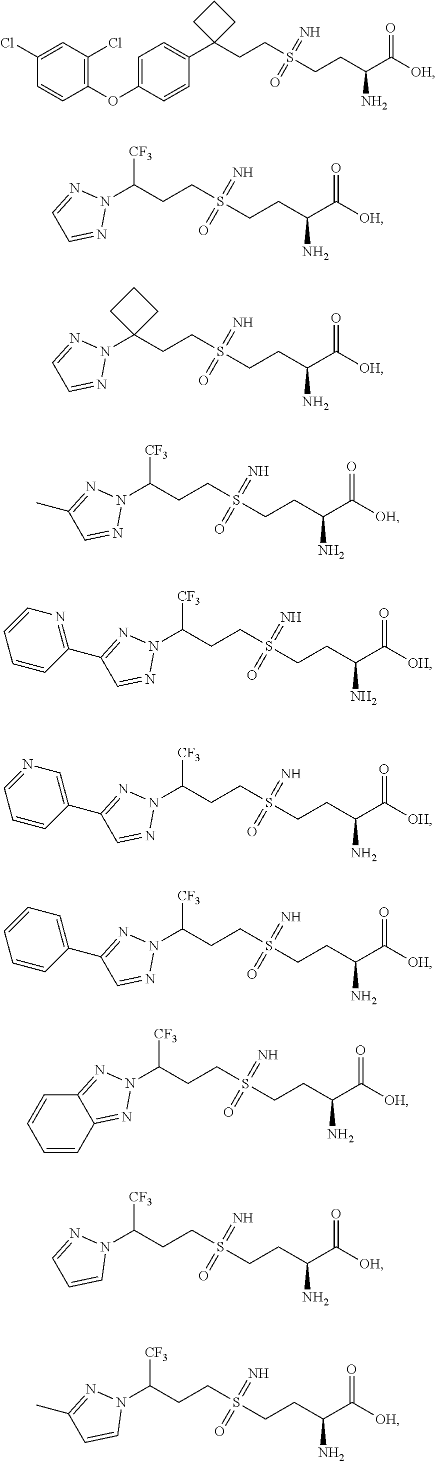

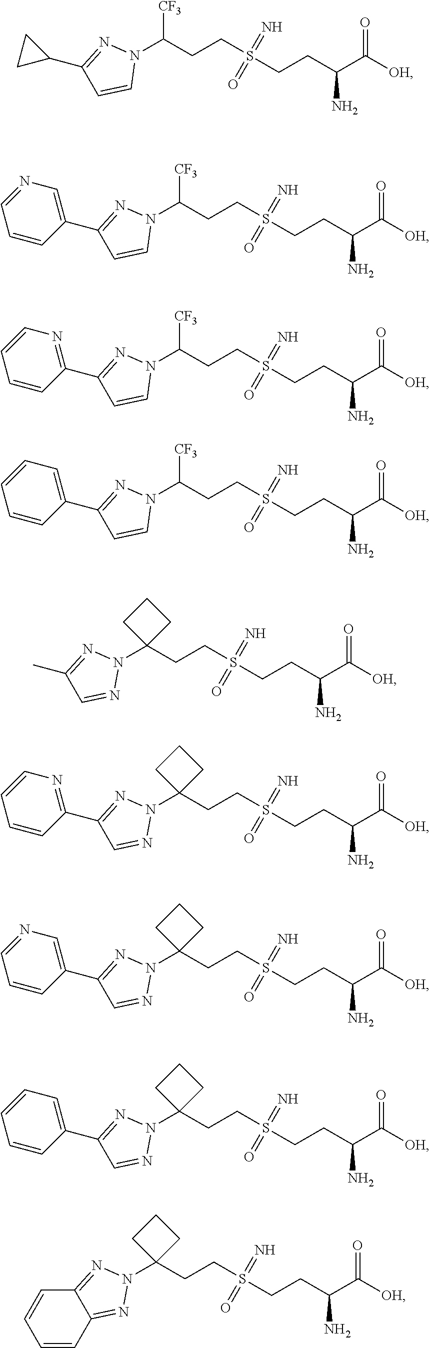

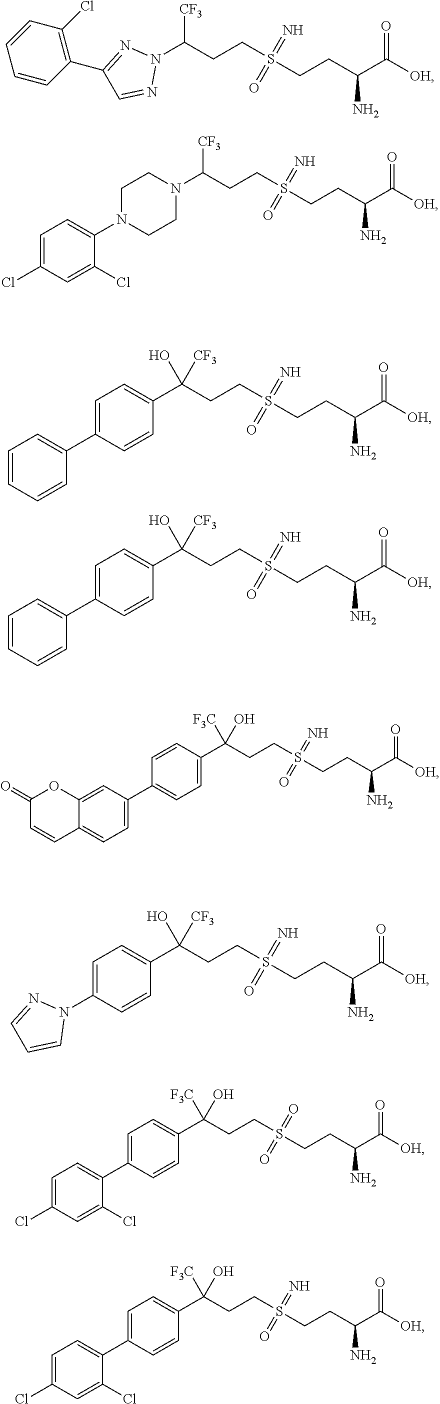

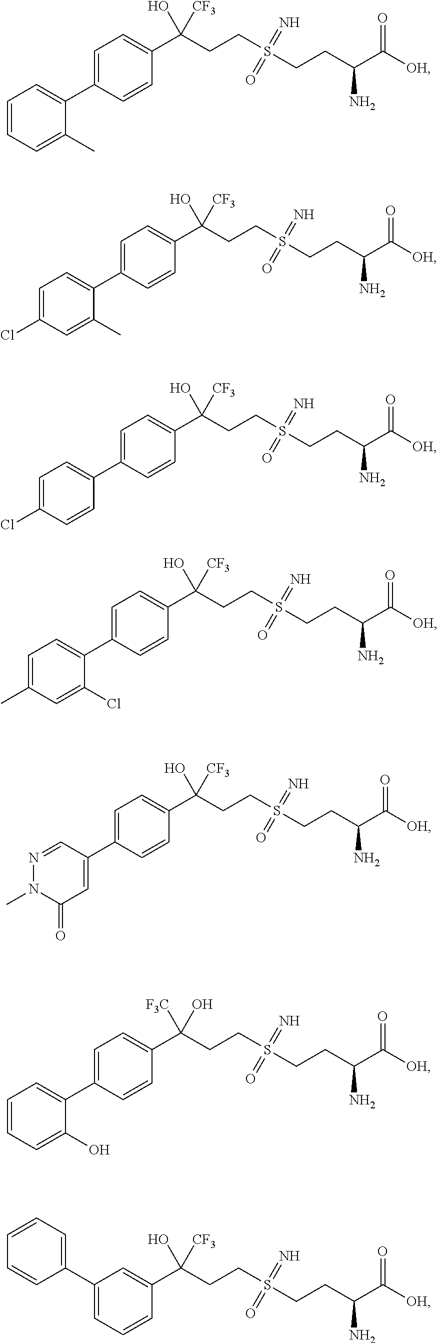

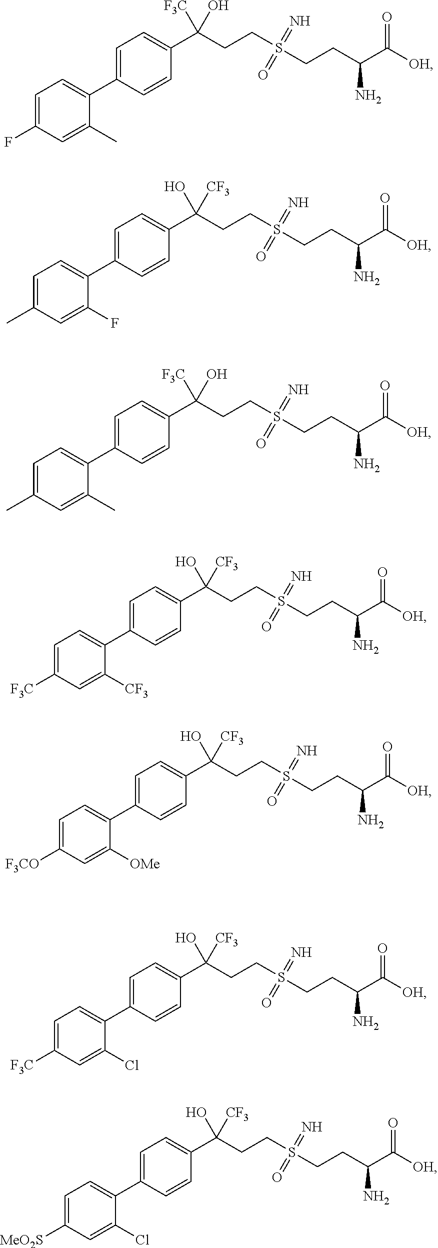

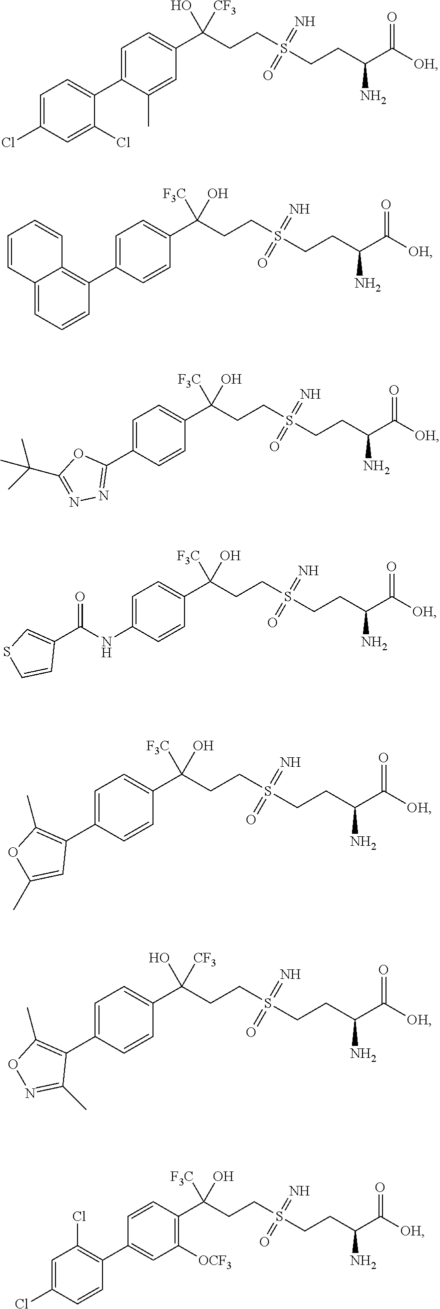

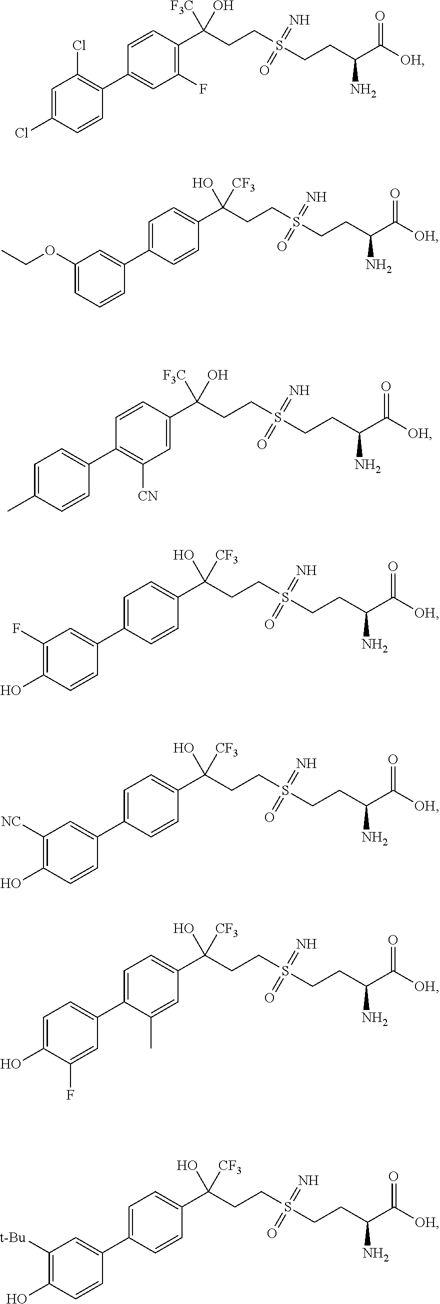

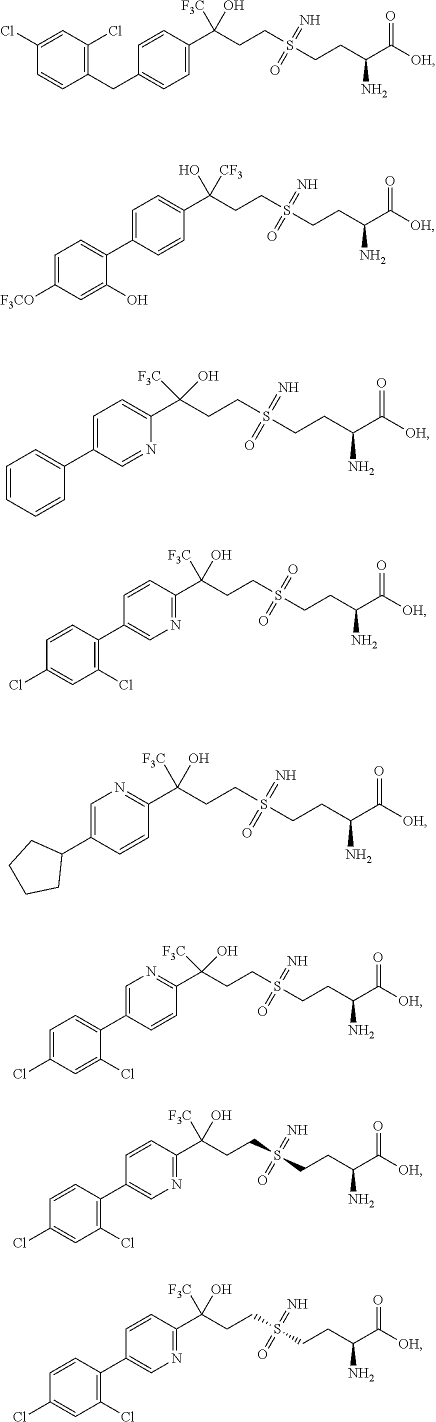

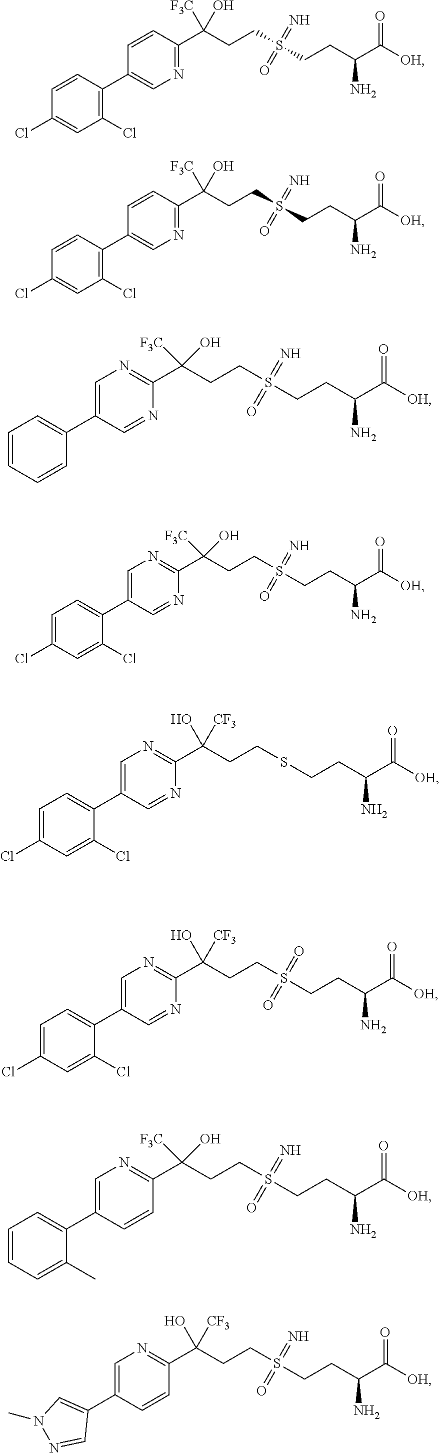

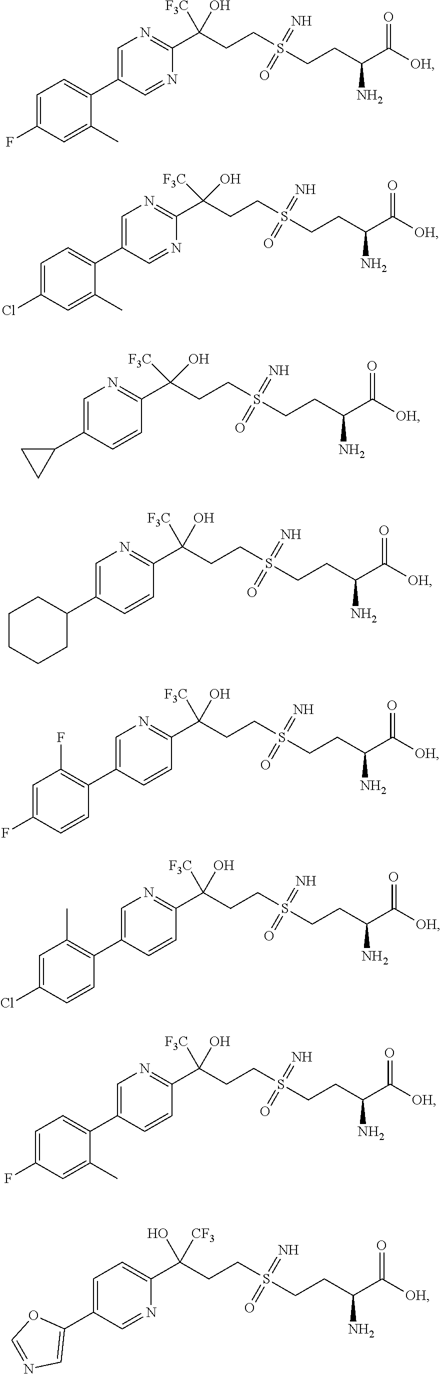

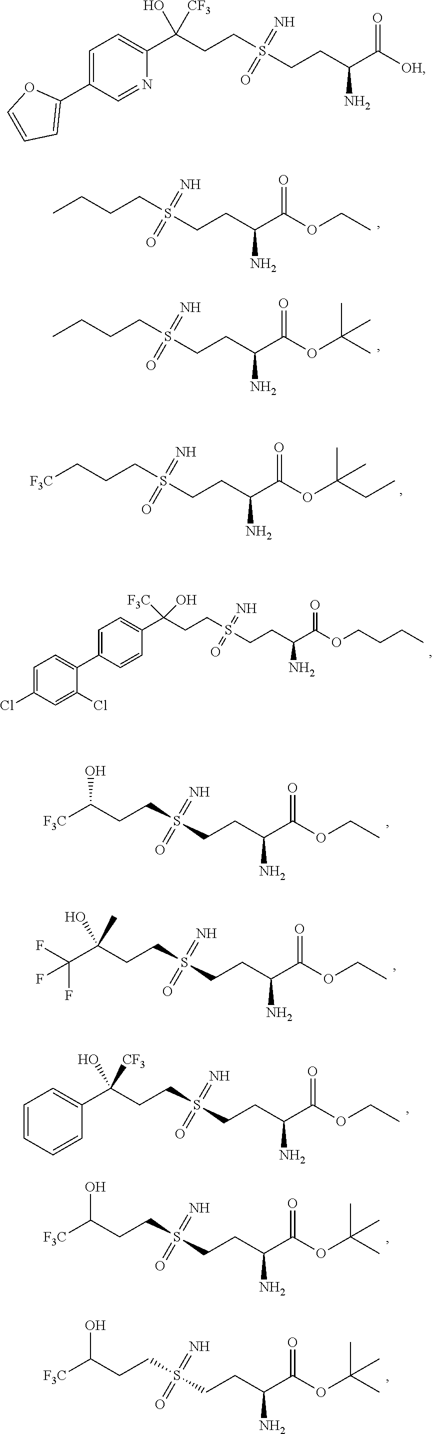

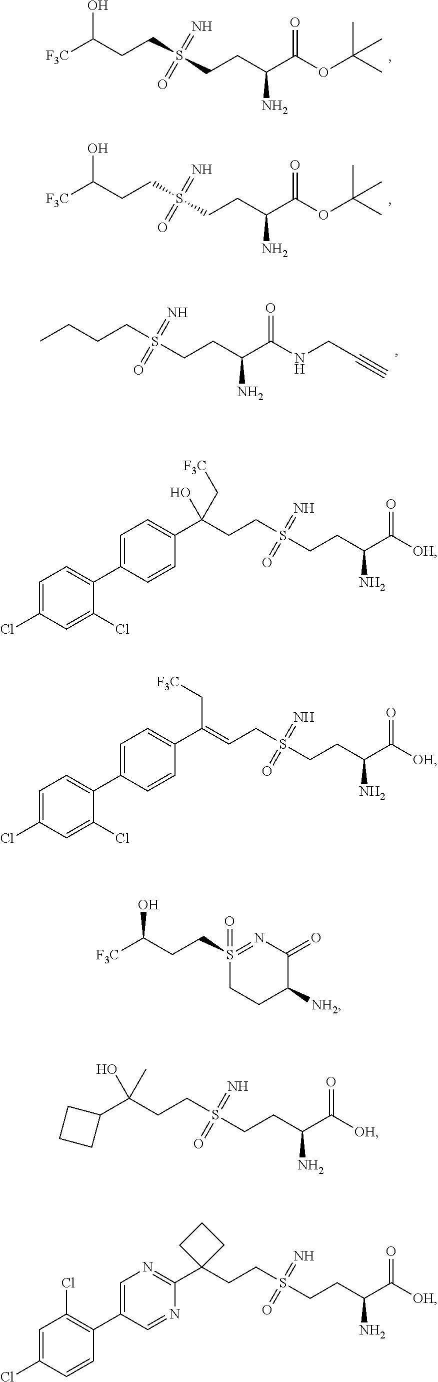

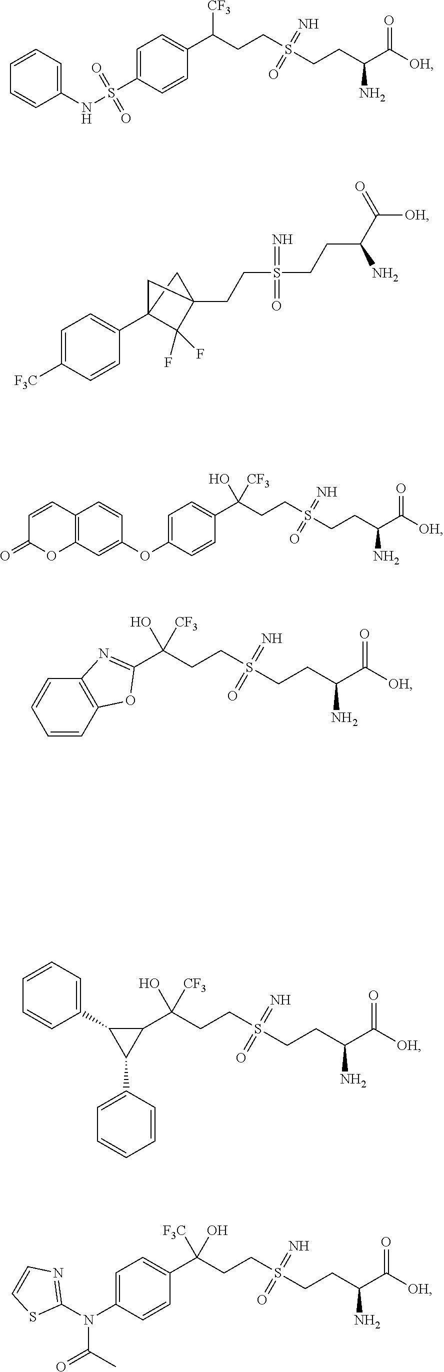 [SMILES: N=S(=O)(CCC(c1ccc(S(=O)(=O)Nc2ccccc2)cc1)C(F)(F)F)CC[C@H](N)C(=O)O ; N=S(=O)(CC[C@H](N)C(=O)O)CCC12CC(c3ccc(C(F)(F)F)cc3)(C1)C2(F)F ; N=S(=O)(CC[C@H](N)C(=O)O)CCC(O)(c1ccc(Oc2ccc3ccc(=O)oc3c2)cc1)C(F)(F)F ; N=S(=O)(CC[C@H](N)C(=O)O)CCC(O)(c1nc2ccccc2o1)C(F)(F)F ; N=S(=O)(CC[C@H](N)C(=O)O)CCC(O)(C1[C@@H](c2ccccc2)[C@H]1c1ccccc1)C(F)(F)F ; CC(=O)N(c1ccc(C(O)(CCS(=N)(=O)CC[C@H](N)C(=O)O)C(F)(F)F)cc1)c1nccs1]

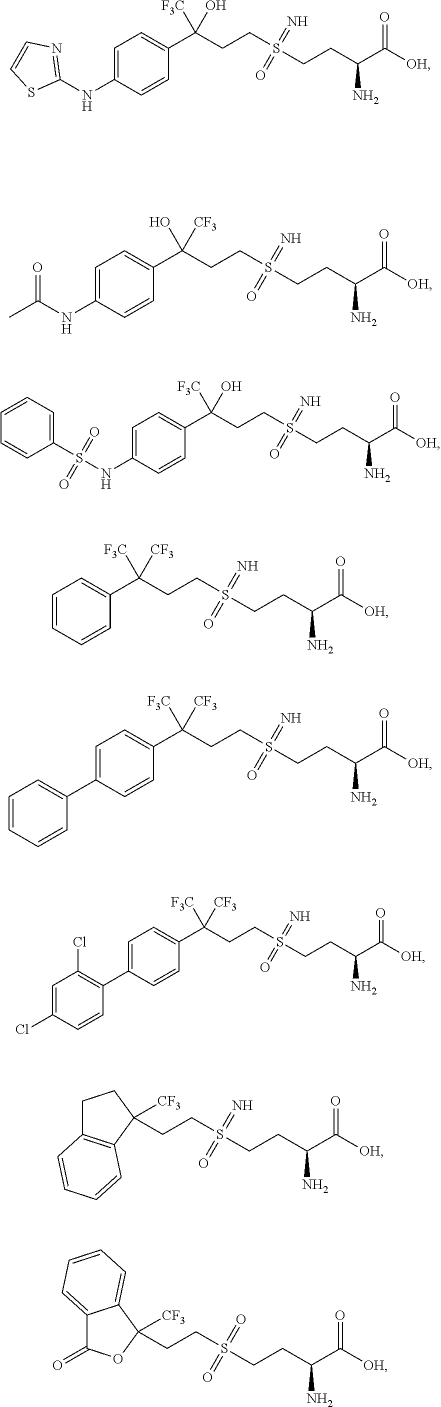

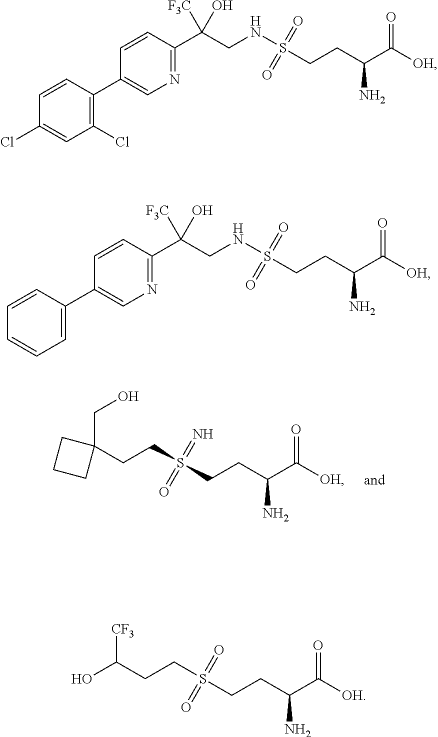 [SMILES: N[C@@H](CCS(=O)(=O)NCC(O)(c1ccc(-c2ccc(Cl)cc2Cl)cn1)C(F)(F)F)C(=O)O ; N[C@@H](CCS(=O)(=O)NCC(O)(c1ccc(-c2ccccc2)cn1)C(F)(F)F)C(=O)O ; N=[S@@](=O)(CC[C@H](N)C(=O)O)CCC1(CO)CCC1 ; N[C@@H](CCS(=O)(=O)CCC(O)C(F)(F)F)C(=O)O]

In some embodiments is a compound, a diastereomer or an enantiomer of the compound, or a pharmaceutically acceptable salt of any of the foregoing, or a deuterated derivative of any of the foregoing; wherein the compound is selected from the group consisting of:

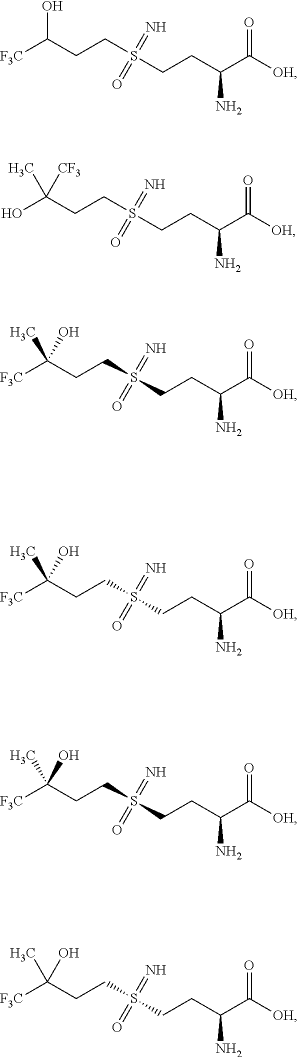 [SMILES: N=S(=O)(CCC(O)C(F)(F)F)CC[C@H](N)C(=O)O ; CC(O)(CCS(=N)(=O)CC[C@H](N)C(=O)O)C(F)(F)F ; C[C@@](O)(CC[S@](=N)(=O)CC[C@H](N)C(=O)O)C(F)(F)F ; C[C@@](O)(CC[S@@](=N)(=O)CC[C@H](N)C(=O)O)C(F)(F)F ; C[C@](O)(CC[S@](=N)(=O)CC[C@H](N)C(=O)O)C(F)(F)F ; CC(O)(CC[S@@](=N)(=O)CC[C@H](N)C(=O)O)C(F)(F)F]

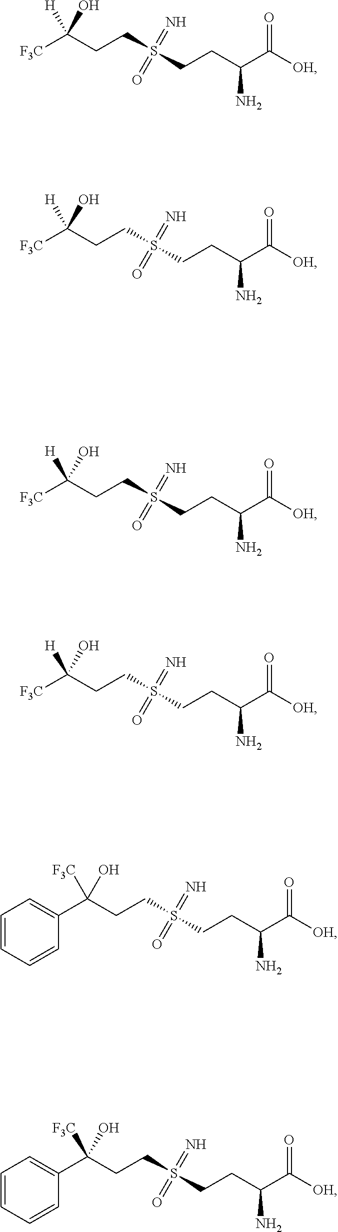 [SMILES: N=[S@@](=O)(CC[C@H](N)C(=O)O)CC[C@H](O)C(F)(F)F ; N=[S@](=O)(CC[C@H](N)C(=O)O)CC[C@H](O)C(F)(F)F ; N=[S@](=O)(CC[C@@H](O)C(F)(F)F)CC[C@H](N)C(=O)O ; N=[S@@](=O)(CC[C@@H](O)C(F)(F)F)CC[C@H](N)C(=O)O ; N=[S@](=O)(CC[C@H](N)C(=O)O)CCC(O)(c1ccccc1)C(F)(F)F ; N=[S@@](=O)(CC[C@H](N)C(=O)O)CC[C@@](O)(c1ccccc1)C(F)(F)F]

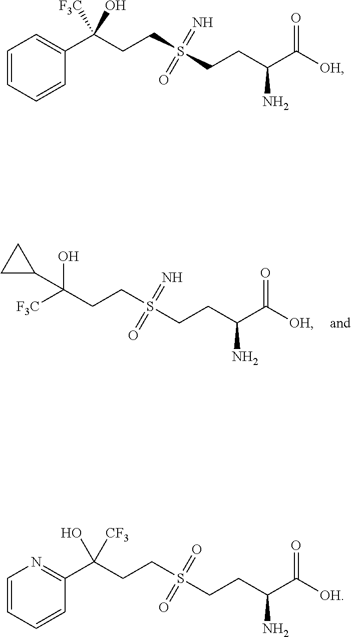 [SMILES: N=[S@@](=O)(CC[C@H](N)C(=O)O)CC[C@](O)(c1ccccc1)C(F)(F)F ; N=S(=O)(CC[C@H](N)C(=O)O)CCC(O)(C1CC1)C(F)(F)F ; N[C@@H](CCS(=O)(=O)CCC(O)(c1ccccn1)C(F)(F)F)C(=O)O]

In some embodiments described herein is a compound of Formula XVIII:

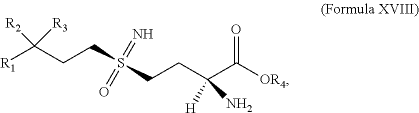

a diastereomer or an enantiomer of the compound of Formula XVIII, or a pharmaceutically acceptable salt of any of the foregoing, or a deuterated derivative of any of the foregoing; wherein in the compound of Formula XVIII:
   each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —CH₃, phenyl, —C(O)OR₅, —C(O)NH₂, —O—, —S—, —OH, —NH₂, —NH—, a halogen, —CF₃, —CN, —F, —Cl, —Br, —I;
   or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F, or R₁ and R₂ and R₃ are taken together to form a C₃-C₆ cycloheteroaryl; and wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

In some embodiments is a compound of Formula XIX:

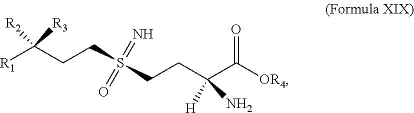

a diastereomer or an enantiomer of the compound of Formula XIX, or a pharmaceutically acceptable salt of any of the foregoing, or a deuterated derivative of any of the foregoing; wherein in the compound of Formula XIX:
   each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —C(O)OR₅, —C(O)NH₂, —O—, —S—, —OH, —NH₂, —NH—, a halogen, —CF₃, —CN, —F, —Cl, —Br, —I,
or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F,
or R₁ and R₂ and R₃ are taken together to form a C₃-C₆ cycloheteroaryl; and wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

In some embodiments is a compound of Formula XX:

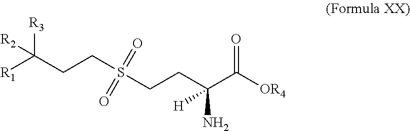

a diastereomer or an enantiomer of the compound of Formula XX, or a pharmaceutically acceptable salt of any of the foregoing, or a deuterated derivative of any of the foregoing; wherein in the compound of Formula XX:
   each R₁, R₂, or R₃ is independently: H, a C₁-C₄ linear or branched alkyl, a C₃-C₆ cycloalkyl, a C₆-C₁₀ aryl, a C₅-C₆ heteroaryl, a biphenyl, a halogenated biphenyl, a triazole, an isothiazole, an oxazoline, a C₁-C₆ linear or branched alkyl ether, —C(O)OR₅, —OH, —NH₂, —NH—, a halogen, —CF₃, —CN, —F,
   or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F; and
   wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, an oxazoline, a C₃-C₆ heteroaryl, and any combination of these;
   wherein R₄ is H, or a C₁-C₆ linear or branched alkyl; and
   wherein R₅ is a C₁-C₁₀ linear or branched alkyl optionally substituted with at least one heteroatom, a halogen, or a C₁-C₃ alkyl ether.

In some embodiments are pharmaceutical compositions comprising the compound of Formula XVIII, XIX, XX, or a compound described herein, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. In some embodiments, the pharmaceutical composition is in unit dose form. In some embodiments, the pharmaceutical composition further comprises an additional active agent or a pharmaceutically acceptable salt thereof. In some embodiments, the pharmaceutical composition is in the form of a powder, a tablet, a capsule, a liquid, or a gel. In some embodiments, the pharmaceutical composition is present in the pharmaceutical composition in an amount ranging from about 0.001 mg to about 25,000 mg. In some embodiments, are kits comprising a pharmaceutical composition described herein, and a container. In some embodiments, the container is a syringe. In some embodiments, the container is an IV bag. In some embodiments, the container is disposable. In some embodiments, the container is a single use container. In some embodiments, the container is a resealable container.

Also described herein are methods of treating a cancer in a subject. In some embodiments, the method comprises administering a pharmaceutical composition described herein to the subject in a therapeutically effective amount, thereby treating the cancer. In some embodiments, method comprising administering to the subject the compound of Formula XVIII, Formula XIX, Formula XX, or a compound described herein; the diastereomer or the enantiomer of any of the foregoing, or the pharmaceutically acceptable salt of any of the foregoing, or the deuterated derivative of any of the foregoing; in a therapeutically effective amount, thereby treating the cancer. In some embodiments, the cancer is a carcinoma, a sarcoma, or a melanoma. In some embodiments, the carcinoma is a liver carcinoma. In some embodiments, the cancer is a clear cell renal carcinoma or non-clear cell renal carcinoma. In some embodiments, the cancer is an SWI/SNF deficient-complex cancer. In some embodiments, the administering is selected from the group consisting of: oral, an injection; subcutaneous, intra-tumoral; systemic, local, intravenous, intraperitoneal, intramuscular, and any combination thereof.

Also described herein are methods of modulating ferroptosis in a tissue, the method comprising contacting the tissue with a pharmaceutical composition described herein in an amount effective to modulate the ferroptosis in the tissue. In some embodiments, the tissue is comprised in a subject. In some embodiments, the subject is a subject in need thereof. In some embodiments, the subject is a mammal. In some embodiments, the subject is a human. In some embodiments, the administering or the contacting is: as needed, once per day, twice per day, three times per day, once per week, once per two weeks, once per three weeks, once per month, once every six months, once per year, or for life. In some embodiments, the therapeutically effective amount, or the amount effective, ranges from about 0.001 mg to about 25,000 mg.

Also described herein are methods of treating a disease or condition in a subject, the method comprising administering a pharmaceutical composition described herein to the subject in a therapeutically effective amount, thereby treating the disease or condition. Also described herein are methods of treating a disease or condition in a subject, the method comprising administering a pharmaceutical composition described herein, the diastereomer or the enantiomer of any of the foregoing, or the pharmaceutically acceptable salt of any of the foregoing, or the deuterated derivative of any of the foregoing; in a therapeutically effective amount, thereby treating the disease or condition. In some embodiments, the disease or condition is a fibrosis or a kidney disorder.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic representation of the ferroptosis pathway.

FIGS. 2A-2B demonstrate tumor response after exposure to (1) BSO or (2) BSO+lip-1 or fer-1 over time. FIG. 2A shows cleaved caspase-3 staining. FIG. 2B shows a graph demonstrating the fractional viability (y-axis) of cells over time (x-axis) for BSO and for BSO+fer-1.

FIG. 3 demonstrates tumor response after exposure to (1) ML-210 or (2) ML-210+lip-1 after 24 hours. Drug were loaded to achieve concentrations of 1-10 μM for both ML-210 and lip-1. Staining shows cleaved caspase-3. Dashed lines indicate region of drug exposure. Scale bar: 100 micrometers (μm).

FIG. 4 demonstrates dose-response curves for BSO and RSL3 on fractional cell viability (y-axis) normalized to DMSO. The x-axis shows drug concentration.

FIGS. 5A-5B demonstrate tumor response after exposure to (1) RSL3 or (2) RSL3+lip-1 after 24 hours. Drug were loaded to achieve concentrations of 1-10 μM for both RSL3 and lip-1. FIG. 5A shows a tumor section stained for cleaved caspase-3. Dashed lines indicate region of drug exposure. Scale bar: 100 micrometers (μm). FIG. 5B shows representative H&E images at 18 hrs post treatment with (1) RSL3 or (2) RSL3+lip-1 as indicated.

FIGS. 6A-6B are structural representations of compound 322 analyzed by x-ray crystallography. FIG. 6A shows the absolute configuration of compound 322. FIG. 6B shows the ORTEP structure of compound 322.

FIGS. 7A-7B are structural representations of compound 324 analyzed by x-ray crystallography. FIG. 7A shows the absolute configuration of compound 324. FIG. 7B shows the ORTEP structure of compound 324.

FIGS. 8A-8B are structural representations of a hydrate of compound 328 analyzed by x-ray crystallography. FIG. 8A shows the absolute configuration of a hydrate of compound 328. FIG. 8B shows the ORTEP structure of a hydrate of compound 328.

FIGS. 9A-9B are structural representations of compound 348 analyzed by x-ray crystallography. FIG. 9A shows the absolute configuration of compound 348. FIG. 9B shows the ORTEP structure of compound 348.

### DETAILED DESCRIPTION OF THE DISCLOSURE

The following description and examples illustrate embodiments of the disclosure in detail. It is to be understood that this disclosure is not limited to the particular embodiments described herein and as such can vary. There are numerous variations and modifications herein, which are encompassed within the disclosure.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

Unless specifically stated or obvious from context, as used herein, the term “about” is understood as within a range of normal tolerance in the art, for example within 2 standard deviations of the mean. “About” can be understood as within plus or minus: 15%, 14%, 13%, 12%, 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Where particular values are described in the application and claims, unless otherwise stated, the term “about” is implicit and in this context means within an acceptable error range for the particular value.

Unless otherwise stated, structures depicted herein are also meant to include all isomeric (e.g., enantiomeric, diastereomeric, geometric (or conformational) forms of the structure; for example, the L and D designations for each asymmetric center, the R and S configurations for each asymmetric center, (Z) and (E) carbon-carbon double bond isomers, R and S configurations for each sulfoximine sulfur atom center, and (Z) and (E) conformational isomers. Therefore, single stereochemical (enantiomers, diastereomers) isomers (enantiomers, diastereomers) as well as enantiomeric, diastereomeric, and geometric (or conformational) mixtures of the present compounds are within the scope of the disclosure. Each independent stereocenter, unless explicitly defined, may include, a mixture of stereoisomers, or a pure stereoisomer, thereof.

Unless otherwise stated, compounds with one or more asymmetric centers referred to herein, include enantiopure, diastereomeric, diastereopure, enantioenriched, diastereoenriched, and racemic mixtures thereof.

The term “adjacent” and its grammatical equivalents as used herein refer to right next to the object of reference. For example, the term adjacent in the context of a cell or a tissue can mean without any other cells or tissues in between.

The following structural aspect:

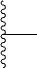

is used in structural formulas herein to depict the bond that is the point of attachment of the moiety or substituent to the core or backbone structure.

The term “analog” and its grammatical equivalents as used herein refer to a molecule that is not identical but has analogous structural features. An analog of a drug or agent is a drug or agent that is related to a reference agent, but whose chemical structure can be different. Analogues exhibit similar activities to a reference drug or agent, but the activity can be increased or decreased or otherwise improved. An analogue form of a compound or drug can mean that the backbone core of the structure is modified or changed compared to a reference drug.

The term “prodrug” as used herein is a first molecule that undergoes a chemical change after administration to a subject to form a second molecule, where the second molecule is a biologically active agent.

The term “anti-cancer agent” or “chemotherapeutic agent” and its grammatical equivalents as used herein refer to an agent that is capable of killing cells that divide rapidly (e.g., cancer cells), preventing the cells that divide rapidly from further dividing, of slowing the division of rapidly dividing cells. Exemplary anti-cancer agents provided herein can include ferroptosis inducing agents, can be used be used in combination with one or more additional ferroptosis inducing agents, can be used in combination with an iron-dependent cell death inducing agent, and/or can be used in combination with a second therapeutic agent or second active agent. The second therapeutic agent or second active agent can be in the form of a prodrug. The second therapeutic agent or second active agent can be in the form of a pharmaceutically acceptable salt. The second therapeutic agent or second active agent can be an alkylating agent such as a nitrogen mustard, can be chloramcucil, cyclophosphamide, isofamide, melphalan, or bisulfan; a nitrosourea, which can be, for example, streptozocin, carmustine, or lomustine; an alkyl sulfonate such as busulfan; a triazine, such as dacarbazine or temozolomide; or an ethylenimine, such as thiotepa- or altretamine. The second therapeutic agent or second active agent can be an antimetabolite, which can be a purine antagonist, a pyrimidine antagoinist or a folate antagoinist, for example, 5-fluorouracil, 6-mercaptopurine, capecitabine, cladribine, or clofarabine. The second therapeutic agent or second active agent can be an anti-tumor antibiotic. The second therapeutic or second active agent can be a mitotic inchibitor. The second threapeutic or second acrive agent can be a corticosteroid. The second therapeutic agent or second active agent can be a plant alkaloid, for example, actinomycid D, a doxorubicin, or a mitomycin, such as mitomycin C. The second therepatuic agent or second active agent can be an antitumor antibiotic, for example, a doxorubicin, a mitoxantrone, or a bleomycin. The second therapeutic or the second active agent can be, for example, mechlorethamine, leucovorin, methotrexate, mercaptopurine, busulfan, chlorambucil, cyclophosphamide, vincristine, dactinomycin, vinblastine, thioguanine, procarbazine, floxuridine, fluorouracil, mitotane, bleomycin, doxorubicin, dacarbazine, lomustine, carmustine, cisplatin, asparaginase, streptozocin etoposide, ifosfamide, carboplatin, altretamine, fludarabine, pentostatin, paclitaxel, melphalan, teniposide, cladribine, vinorelbine, pegaspargase, thiotepa, docetaxel, gemcitabine, irinotecan, toptecan, idarubicin, capecitabine, daunorubicin, valrubicin, temozolomide, cytarabine, epirubicin, arsenic trioxide, mitomycin, oxaliplatin, pemetrexed disodium, clofarabine, nelarabine, ixabepilone, bendamusting hydrochloride, paratrexate, carbazitazel, erbulin mesylate, asparaginaseerwinia chrsanthemi, omacetaxine mepesuccinate, radium 223 dichloride, fluoxymesterone, methyltestosterone, tamoxifen, tamoxifen citrate, estramustine, interferon alpha 2b (recombinant), gosrelin, flutamide, aldeslukin, bicalutamide, anastrozole, porfimer, nilutamide, imiquimod, letrazole, rituximab. Toremifene, thalidomide, trastuzmad, alitretinonin, bexarotene, denileukin diftitox, exemestane, gemfluzumab ozogamicin, exemestane, gemtuzumab ozogamicin, triptorelin, alemtuzamub, imatinib, imatinim mesylate, peginterferon alpha 2-B, fulvestrant, iron, an iron comprising nanoparticle, ibritumomab tiuxetan, leuprolide, leuprolide acetate, abarelix, bortezomib, genfitinib, tositumomab and iodine I 131, tositumomab, bevacizumab, cetuximab, erlotinib, erlotinib hydrochloride, lenalidomide, sorafenib, sorafenib tosylate, dasatinib, decitabine, panitumamab, sunitinib, sunitinib malate, vorinostat, lapatinib, lapatinib ditosylate, nilotinib, temsirolimus, degarelix, everolimus, ofatumumab, pazopanib, pazopanib hydrochloride, romidepsin, denosumab, hydroxyurea, spuleucel-T, abiraterone, abiratone acetate, brentuximab vedotin, crizotinib, iplimumab, ruxolitinib, ruxolitinib phosphate, vandetanib, vemurafenib, pertuzumab, axitinib, bosutinib, carbozantinib, carfilzomib, enzalutamide, ponatinib, ponatinib hydrochloride, regorafenib, vismodegrib, ziv-aflibercept, dabrafenib, trametinib, obinutuzumab, adotrastuzumab emtansine, afatinib, ibrutinib, pomalidomide, idelalisib, belinostat, ceritinib, perbrolizumab, ramucirumab, lanreotide, blinatumomab, nivolumab, olaparib, a checkpoint inhibitor, ipilimumab, nivolumab, pembrolizumab, atezolizumab, avelumab, durvalumab, cemiplimab, a chimeric antigen receptor T cell therapy (CAR-T cell therapy), CAR natural killer cell therapy (CAR NK therapy), tisagenlecleucel, axicabtagene ciloleucel, brexucabtagene autoleucel, lisocabtagene maraleucel, idecabtagene vicleucel, ciltacabtegene autoleucel, a compound of Table 1, an enantiomer or diastereomer thereof, a pharmaceutically acceptable salt of any of the foregoing, or a deuterated derivative of any of the foregoing, or any combination of the foregoing

The term “cancer” and its grammatical equivalents as used herein refer to a hyperproliferation of cells whose unique trait-loss of normal controls-results in unregulated growth, lack of differentiation, local tissue invasion, and metastasis. With respect to the methods provided herein, the cancer can be any cancer, including but not limited to any one of, acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumors, liver cancer, lung cancer, lymphoma, malignant mesothelioma, mastocytoma, melanoma, dedifferentiated melanoma, multiple myeloma, nasopharynx cancer, non-Hodgkin lymphoma, ovarian cancer, pancreatic cancer, peritoneum, omentum, and mesentery cancer, pharynx cancer, prostate cancer, colorectal cancer, renal cancer, a carcinoma, renal carcinoma, non-clear cell renal carcinoma, clear cell renal carcinoma, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type. Any cancer or neoplastic condition, tumor, or population of cancerous cells can be SWI/SNF deficient. In some instances, any cancer or neoplastic condition, tumor, or population of cancerous cells is not SWI/SNF deficient.

The term “drug resistant cancer” and its grammatical equivalents as used herein refers to a cancer that does not respond, or exhibits a decreased response to, one or more chemotherapeutic agents.

The term “effective amount” or “therapeutically effective amount” and its grammatical equivalents refers to an amount that is sufficient to achieve or at least partially achieve the desired effect.

The term “expression” and its grammatical equivalents as used herein refers to the biosynthesis of a gene product. For example, in the case of a structural gene, expression involves transcription of the structural gene into mRNA and the translation of mRNA into one or more polypeptides.

The term “ferroptosis” refers to a form of cell death involving generation of reactive oxygen species mediated by iron, and characterized by, in part, lipid peroxidation. The term “ferroptosis-inducing agent” or “ferroptosis activator” or “ferroptosis inducer” or “ferroptosis-inducing compound” or “ferroptosis modulator” refers to an agent which promotes or activates or modulates ferroptosis in a cell.

In some embodiments, a compound or a salt thereof may comprise an enantiomerically pure form. In some examples, the compound or salt thereof disclosed herein can have an enantiomeric excess greater than about or equal to: 5%, 10%, 15%, 20%, 25%, 30%, 35%, 40%, 45%, 50%, 60%, 70%, 80%, 90%, 95%, 96%, 97%, 98%, or 99%. A compound or a salt thereof may be dosed in their enantiomerically or diasteriomerically pure form. In some cases, percent enantiomeric excess can be defined as: % ee=(|F_{R}−F_{S}|×100) wherein F_{R} is the mole fraction of the compound with an R stereocenter and F_{S} is the mole fraction of the compound with an S stereocenter and the two vertical lines indicate taking the absolute value of the difference.

The diastereomer excess, or de (diastereomeric excess) value, can indicate the excess of a diastereomer in a diastereomer mixture. It can be defined as:

$$de = \frac{{m}_{1} - {m}_{2}}{{m}_{1} + {m}_{2}} \cdot 100 \%$$

with: m₁ being mass of the diastereomer in excess, and m₂ being mass of the diastereomer in deficit. In some examples, the compound as a diastereomer or a salt thereof or pharmaceutically acceptable salt thereof or deuterated derivative thereof can have a diasteriomeric excess greater than about or equal to: 5%, 10%, 15%, 20%, 25%, 30%, 35%, 40%, 45%, 50%, 55%, 60%, 65%, 70%, 75%, 80%, 90%, 95% or 99%. With a 1:1 mixture of two diastereomers, de=0%, with a diastereomerically pure compound de=100%.

The term “hyperproliferative cells” and its grammatical equivalents as used herein refers to cells characterized by unwanted cell proliferation, or abnormally high rate or sustained cell division, unrelated or uncoordinated with that of surrounding normal tissue.

The term “in vitro” and its grammatical equivalents as used herein refers to events that occur in an artificial environment, e.g., in a test tube or reaction vessel, in cell culture, etc., rather than within a multi-cellular organism.

The term “in vivo” and its grammatical equivalents as used herein refers to events that occur within a multi-cellular organism, such as a non-human animal.

The term “iron-dependent cell death agent” and its grammatical equivalents as used herein refers to an agent which induces, promotes or activates cell death mediated by iron. In some cases, within the disclosure, the term “iron-dependent cell death agent” is used interchangeably with ferroptosis-inducing agent.

The term “normal cells” and its grammatical equivalents as used herein refers to cells that undergo controlled cell division, controlled activation, or quiescent cells.

The compounds herein are intended to include all isotopes of atoms occurring in the compounds herein. Isotopes include those atoms having the same atomic number but different mass numbers. By way of general example and without limitation, isotopes of hydrogen include deuterium (D) and tritium (T). Isotopes of carbon can include ¹³C, ¹⁴C, ¹⁵N, ³¹P, or ³²P. Isotopically labeled compounds can generally be prepared using an appropriate isotopically labeled reagent in place of the non-labeled reagent otherwise employed. For example, methyl groups also include deuterated methyl groups such as —CD₃.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Compounds described herein may be depicted to show stereochemistry using hashed or wedged bonds as shown below. For compounds with a stereogenic atom, when only one substituent is hashed or wedged, a 4^{th} substitutent can be interpreted to have the opposite orientation in space. Accordingly, the two exemplary structures shown below can be interpreted interchangeably.

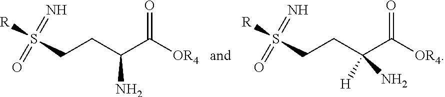

For compounds with a stereogenic sulfoximine sulfur atom, when two substituents are both hashed or both wedged, the third and fourth substituents can be interpreted to have the opposite orientation in space. Accordingly, the two exemplary structures shown below can be interpreted interchangeably. The left structure comprises an oxo group into the plane, a double-bonded NH group into the plane, an R group out of the plane, and a saturated carbon substituent out of the plane.

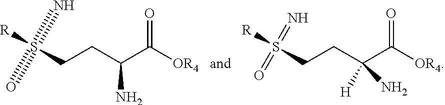

### Overview

Provided herein are compounds, compositions containing the compounds, and pharmaceutical compositions containing the compounds, and methods using these for treating a disease or condition in a subject. The compounds can be or comprise one or more compounds of Formula I, one or more compounds of Formula II, enantiomers of any of these, diastereomers of any of these, pharmaceutically acceptable salts of any of these, or deuterated derivatives of any of these, optionally in combination with a second therapeutic agent or second active agent. The compositions or pharmaceutical compositions can contain one or more of any of these. The disease or condition can be a cancer, for example in a tissue of the subject. The cancer can be comprised in a mammal, or contained in a tissue of a mammal, which can be a human, which can be male, female. The disease or condition can be an inflammatory disease, or a fibrosis, and the subject can in need thereof and can be a mammal, a human, a female, or a male.

Also provided herein are compounds, compositions containing the compounds, pharmaceutical compositions containing the compounds, and methods of using these for modulating, inhibiting, or partially inhibiting a target comprising glutamate-cysteine ligase (GCL), for example in a subject, optionally in a tissue. The compounds can be or comprise one or more compounds of Formula I, one or more compounds of Formula II, enantiomers any of these, diastereomers any of these, pharmaceutically acceptable salts of any of these, or deuterated derivatives of any of these, optionally in combination with a second therapeutic agent or second active agent. The subject can be in need thereof of can be a mammal, a human, a female, or a male.

Also provided herein are compounds, compositions comprising the compounds, and pharmaceutical compositions comprising the compounds, and methods of making the compounds and compositions comprising the compounds, and methods of using these for modulating or inducing ferroptosis, optionally in a subject, optionally in a tissue. The compounds can be or comprise one or more compounds of Formula I, one or more compounds of Formula II, enantiomers thereof, diastereomers thereof, pharmaceutically acceptable salts of any of these, or deuterated derivatives of any of these, optionally in combination with a second therapeutic agent or second active agent. The subject in need thereof of can be a mammal, a human, a female, or a male.

Also provided herein are treatment regimens for the therapy of various diseases or conditions such as cancer, an inflammatory disease, or a fibrosis, or for modulating, inhibiting, or partially inhibiting GCL, or for modulating or inducing ferroptosis in a subject. A treatment regime can comprise administering a compound of Formula I, a compound of Formula II, enantiomers any of these, diastereomers of any of these, pharmaceutically acceptable salts of any of these, or deuterated derivatives of any of these, optionally in combination with a second therapeutic agent or second active agent. Briefly, further described herein are (1) methods of characterizing ferroptosis-sensitive cells; (2) cell death-inducing agents including ferroptosis-inducing agents, and chemotherapeutic agents; (3) pharmaceutical compositions; (4) dosing; (5) methods of administration; (6) efficacy; (7) therapeutic applications; and (8) systems.

The compounds, enantiomers thereof, diastereomers thereof, pharmaceutically acceptable salts of any of the foregoing, or deuterated derivatives of any of the foregoing, can be sulfoximines. The sulfoximines may, in some instances, not comprise BSO.

A subject herein can be a subject in need thereof, can be a mammal, can be a human, and can be a male or female. A subject herein can be diagnosed with a disease or condition prior to being treated, administered, or contacted with a compound of Formula I, a compound of formula II, an enantiomer or a diastereomer of any of the foregoing, a pharmaceutically acceptable salt of any of the foregoing, a deuterated derivative of any of the foregoing, or a composition or pharmaceutical composition comprising any of the foregoing. The diagnosis can be from an in vitro diagnostic, or an in vitro diagnostic which can be a companion diagnostic.

When two or more compounds of: Formula I, Formula II, an enantiomer of any of the foregoing, a diastereomer of any of the foregoing, a pharmaceutically acceptable salt of any of the foregoing, or deuterated derivative of any of the foregoing are contained in a composition or a pharmaceutical composition, the composition can be a fixed dose combination drug.

When two, three, four, five, six, seven, eight, nine, or ten: compounds, therapeutic agents, second therapeutic agents, or second active agents herein are administered to a cell, a tissue, or a subject, the compounds can be administered concurrently or consecutively. When administered concurrently, the administration can be in a single composition or pharmaceutical composition, which can be a fixed dose combination drug.

Compounds, for example of Formula I and Formula II, agents, therapeutics enantiomers or diastereomers of any of these, salts and pharmaceutically acceptable salts of any of these, and deuterated derivatives of any of these, can independently be administered continuously or discontinuously. Compounds, for example of Formula XVIII and Formula XIX, and Formula XX, agents, therapeutics enantiomers or diastereomers of any of these, salts and pharmaceutically acceptable salts of any of these, and deuterated derivatives of any of these, can independently be administered continuously or discontinuously. When administered discontinuously, the administration can be at regularly spaced time intervals or irregularly spaced time intervals. Continuous and discontinuous administration can result in a baseline level of compound or agent being continuously present in a cell, tissue, organ, or system. The baseline level can be achieved, for example, for about: 8, 9, 10, 11, 12, 13 14, 15 16, 17, 18, 19, 20, 21, 22, 23, 24, 25, 25, 27, 28, 29, 30, 31, 32, 33, 34, 35, 36, 37, 38, 39, 40, 41, 42, 43, 44, 45, 46, 47, 48, or more hours. Administration can be independently be by any route of administration, and can be, for example, orally, intravenously, subcutaneous, intramuscular, intraperitoneal, intratumoral, intertumoral, administration to the brain or central nervous system, to the bladder, to an organ or portion thereof, to a tissue or portion thereof, or any combination of these. Compounds, for example of Formula I and Formula II, agents, therapeutics enantiomers or diastereomers of any of these, salts and pharmaceutically acceptable salts of any of these, and deuterated derivatives of any of these, when administered as a solution, can independently have a concentration, for example, or about: 0.01 μM, 0.1 μM 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM 50 μM, 60 μM, 70 μM, 80 μM, 90 μM, 100 μM, 200 μM, 300 μM 400 μM, 500 μM, or more.

In some embodiments, the compound of Formula I, the compound of Formula II, any compound or agent or therapeutic herein, an enantiomer of any of the foregoing, a diastereomer of any of the foregoing, a salt or pharmaceutically acceptable salt of any of the foregoing, or a deuterated derivative of any of the foregoing, can be comprised as a ligand in a proteolysis-targeting chimera (PROTAC) protein degrader. In some instance, a bifunctional PROTAC molecule can comprise the ligand of the protein of interest (POI) and a covalently linked ligand of an E3 ubiquitin ligase (E3). In some instances, the POI can be any protein herein. In some instances, the POI can be cysteine-glutamate antiporter (system Xc), a glutathione peroxidase 4 (GPX4), a p53, a cargo receptor NCOA4, a glutathione synthetase (GSH), or a glutamate-cysteine ligase (GCL). The inactivation or inhibition of some of these molecules, for example, system Xc, GPX4, or glutathione synthetase PROTAC protein degraders can work by recruiting a chosen E3 ligase into close proximity with a specific disease-causing protein so that it can be tagged with ubiquitin and sent off for degradation by the proteasome. After the protein is degraded, the PROTAC can be released to continue to elicit further degradation.

In some embodiments, a compound described herein can be part of an antibody-drug conjugate (ADC) where the compound is optionally linked to the antibody by a linker.

In some instances, the compound of Formula I, the compound of Formula II, any compound or agent or therapeutic herein, an enantiomer of any of the foregoing, a diastereomer of any of the foregoing, a salt or pharmaceutically acceptable salt of any of the foregoing, or a deuterated derivative of any of the foregoing can be delivered directly to a tissue, a tumor, or a cell with a system comprising, for example, a pump, for example a minipump or a syringe pump, and at least one or a needle, a hollow tube, and any combination thereof.

When a sulfoximine is depicted as a chemical structure, for example in the case of the compound of Formula I:

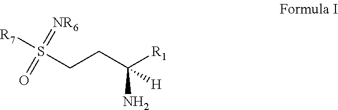

unless explicitly stated otherwise, the R₆ depicts the presence of the substituent R₆ and the imine containing R₆ encompasses (E), (Z), and mixtures of the (E) and (Z) configurations.

### Ferroptosis

Cell death is a cellular process involved in development, cellular homeostasis, and prevention of proliferative diseases such as cancer. Programmed cell death can take different forms, such as apoptosis, mitotic catastrophe, necrosis, senescence, and autophagy. While each of these processes ultimately lead to cell death, the pathways and mechanisms appear to be unique, both at the molecular and cellular level.

Ferroptosis is a non-apoptotic, oxidative form of regulated cell death involving lipid hydroperoxides and the accumulation of lipid peroxide at the cellular plasma membrane. Cells undergoing ferroptosis do not display the cellular characteristics or functions associated with apoptosis, the canonical form of cell death. Examples of apoptotic cell features include, e.g., mitochondrial cytochrome c release, caspase activation, and chromatin fragmentation. Ferroptosis is also characterized by increased levels of intracellular reactive oxygen species (ROS) which can be prevented by iron chelation and genetic inhibition of cellular iron uptake. Addition of iron, but not by other divalent transition metal ions, can potentiate ferroptosis signaling in cells.

Cellular components implicated in and regulating ferroptosis include, among others, cysteine-glutamate antiporter (system Xc), glutathione peroxidase 4 (GPX4), p53, cargo receptor NCOA4, glutathione synthetase (GSH), glutamate-cysteine ligase (GCL). The inactivation or inhibition of some of these molecules, for example, system Xc, GPX4, or glutathione synthetase leads to iron-dependent cell death or ferroptosis.

Hyperproliferative cells in a drug-resistant state, such as, e.g., drug resistant cancer cells have been found to exhibit a dysregulation in apoptosis cellular pathways. Surprisingly, drug-resistance to apoptotic agents by hyperproliferative cells can have an enhanced ability to undergo ferroptosis. Apoptosis-resistant cells can be killed via ferroptosis induction due to their “flammable” ferroptosis-sensitive state.

### Methods of Characterizing Ferroptosis-Sensitive Cells

Provided herein are methods of identifying and characterizing a ferroptosis-sensitive cell in a subject. In some embodiments, the characterizing is performed prior to treatment of a subject with a ferroptosis-inducing agent provided herein. Ferroptosis-sensitive cells can be identified by the following properties provided herein: (1) a concentration of selenium greater than a selenium concentration in a corresponding normal cell; (2) a concentration of iron greater than an iron concentration in a corresponding normal cell; (3) a polyunsaturated fatty acid (PUFA) concentration greater than a PUFA concentration in a corresponding normal cell; (4) a peroxidizability index (PI) greater than a PI in a corresponding normal tissue; and/or (5) the expression of one or more markers indicative of a mesenchymal state, among other morphological and histological characteristics. Methods of measuring analyte concentrations of selenium, iron, and PUFAs include, e.g., mass spectrometry, chromatography, immunoassays, immunosorbent assays, absorbance and colorimetric assays, and microwave plasma-atomic emission spectroscopy. Methods of measuring markers of a mesenchymal cell state include, e.g., immunoassays, polymerase chain reaction (PCR) assays, and sequencing assays.

### (1) Selenium (Se) Concentration and Selenoproteins

Selenium (Se) is a micronutrient that facilitates the synthesis of selenoproteins in a cell. Dietary selenium is found in meat, nuts, cereals, mushrooms, and vegetables. The selenium content in the human body ranges from about 13 milligrams (mg) to 20 mg. Selenium is involved in the cellular process of selenoprotein synthesis and ferroptosis. Selenoproteins are rare proteins that comprise a selenocysteine (Sec) residue in the place of a cysteine. Non-limiting examples of selenoproteins include GPX1, GPX2, GPX3, GPX4, GPX6, TXNRD1, TXNRD2 (TXRD2), TXNRD3, DIO1, DIO2, DIO3, SEPHS2, SEPS1, SEPP1, SEP15, SEPN1 (SELENON), SEPX1, SEPW1 (SELENOW), SEPT1, SELH, SELI, SELK, SELM (SELENOM), SELO, and SELV. Selenoproteins exhibit biochemical activities such as oxidoreduction, selenocysteine synthesis, and/or selenium transport. GPX4 is a phospholipid hydroperoxidase that catalyzes the reduction of hydrogen peroxide and organic peroxides, thereby protecting cells against membrane lipid peroxidation, and oxidative stress. GPX4 is a regulator of the ferroptosis pathway and inhibition of GPX4 induces ferroptotic cell death.

Provided herein are methods of identifying a ferroptosis-sensitive cell in a mammalian tissue by the concentration of selenium. In some embodiments, methods provided herein comprise measuring the concentration of selenium (Se) in a cell, a plurality of cells, or a mammalian tissue. In some embodiments, the Se concentration in a cell or the plurality of cells of the mammalian tissue is greater than the Se concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the Se concentration in the plurality of cells of the mammalian tissue is greater than the Se concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, methods provided herein comprise administering to a mammal an effective amount of a ferroptosis-inducing agent, wherein a plurality of cells of a mammalian tissue have a selenium concentration greater than the selenium concentration of cells of a normal or healthy tissue; and ferroptosis is induced in the plurality of cells.

### (2) Iron Concentration

Ferroptosis is an iron-dependent cellular process and ferroptosis-sensitive cells have increased concentrations of intracellular iron compared with normal cells. Cells treated with deferoxamine (DFO), an iron chelator used for treating iron overload and an agent reported to block ferroptosis, can inhibit cell death. Alternatively, iron loading into cells by treatment with ferric ammonium citrate (FAC) is sufficient to mimic particle treatment and induce ferroptosis in amino acid-starved cells. Increased iron uptake in cells can lead to the depletion of glutathione, conceivably due to increased ROS generation which results in ferroptosis induction.

Provided herein are methods of identifying a ferroptosis-sensitive cell in a mammalian tissue by the concentration of iron. In some embodiments, methods provided herein comprise measuring the concentration of iron or iron oxide in a cell, a plurality of cells, or a mammalian tissue. In some embodiments, the ferroptosis-sensitive cells comprises an increased intracellular concentration of iron that is at least about 7 parts per billion (ppb) or more, about 8 ppb or more, about 9 ppb or more, about 10 ppb or more, about 20 ppb or more, about 30 ppb or more, about 40 ppb or more, about 50 ppb or more, about 60 ppb or more, about 70 ppb or more, about 80 ppb or more, about 90 ppb or more, about 100 ppb or more, about 110 ppb or more, about 120 ppb or more, about 130 ppb or more, about 140 ppb or more, about 150 ppb or more, about 160 ppb or more, up to 170 ppb. In some embodiments, the ferroptosis-sensitive cells comprise an increased intracellular concentration of iron that is at least about 2 micromolar (μM) or higher, 2.5 μM or higher, 3.0 μM or higher, 4.0 μM or higher, 5.0 μM or higher, up to 10 μM higher than that of normal cells. In some embodiments, the iron concentration in a cell or the plurality of cells of the mammalian tissue is greater than the iron concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the iron concentration in the plurality of cells of the mammalian tissue is greater than the iron concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, methods provided herein comprise administering to a mammal an effective amount of a ferroptosis-inducing agent, wherein a plurality of cells of a mammalian tissue has an iron concentration greater than the iron concentration of cells of a normal or healthy tissue; and ferroptosis is induced in the plurality of cells.

### (3) PUFA Status

Apoptosis-resistant cells gain advantages by being in a ferroptosis-sensitive state with high levels of polyunsaturated fatty acids (PUFA). Apoptosis-resistant cells can be killed via ferroptosis induction due to their “flammable” high-PUFA state. The flammable state is defined by high membrane abundance of PUFAs (vs. MUFA, monosaturated fatty acids), which are prone to uncontrolled lipid peroxidation—a radical chain reaction of polyunsaturated fatty acids—that leads to ferroptotic cell death.

PUFAs are categorized as omega-3 (n-3) and omega-6 (n-6) depending on the location of the last double bond with reference to the terminal methyl end of the molecule. Non-limiting examples of PUFAs include: hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, Timnodonic acid), heneicosapentaenoic acid (HPA), docosapentaenoic acid (DPA, Clupanodonic acid), docosahexaenoic acid (DHA, Cervonic acid), tetracosahexaenoic acid (Nisinic acid), tetracosapentaenoic acid, linoleic acid (LA), gamma-linolenic acid (GLA), eicosadienoic acid, dihomo-gamma-linolenic acid (DGLA), arachidonic acid (AA), docosadienoic acid, adrenic acid (AdA), docosapentaenoic acid (Osbond acid), tetracosatetraenoic acid, and tetracosapentaenoic acid. Humans can synthesize all fatty acids utilized by the body except for linoleic acid (LA, C18:2n-6) and alpha-linolenic acid (ALA, C18:3n-3).

Provided herein are methods of identifying a ferroptosis-sensitive cell in a mammalian tissue by the concentration of PUFAs. In some embodiments, methods provided herein comprise administering to a mammal an effective amount of a ferroptosis-inducing agent, wherein a plurality of cells of a mammalian tissue has a polyunsaturated fatty acid (PUFA) concentration greater than the PUFA concentration of cells of a normal or healthy tissue; and ferroptosis is induced in the plurality of cells. In some embodiments, the PUFA concentration in the plurality of cells of the mammalian tissue is greater than the PUFA concentration in cells of healthy or non-malignant tissue of the mammal. In some embodiments, the PUFA concentration in the plurality of cells of the mammalian tissue is greater than the PUFA concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the PUFA concentration in the plurality of cells of the mammalian tissue is greater than the PUFA concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, the PUFA concentration in the plurality of cells of the mammalian tissue is greater than a predetermined PUFA concentration. In some embodiments, the predetermined PUFA concentration is about 5, 10, 15, 20, 25, 30, 35, 40, 45, 50, 55, 60, 65, 70, 75, 80, 85, or 90 mole percent of total lipids. In some embodiments, the predetermined PUFA concentration is about 10-20, 20-30, 30-40, 40-50, 50-60, 60-70, 70-80, or 80-90 mole percent of total lipids. In some embodiments, the predetermined PUFA concentration is about 20 mole percent of total lipids.

### (4) PI Index

Cell membrane composition must contain a sufficient threshold of polyunsaturated fatty acyl chains to support enzymatic and/or non-enzymatic lipid peroxidation. The peroxidizability of polyunsaturated fatty acids (PUFAs) is linearly dependent on the number of doubly allylic positions present in the molecules. The susceptibility of a cellular membrane to lipid peroxidation can be estimated using the peroxidizability index (PI), which is calculated from measured fatty acid composition (%, w/w) as follows: PI=(% dienoic×1)+(% trienoic×2)+(% tetraenoic×3)+(% pentaenoic×4)+(% hexaenoic×5). Alternatively, PI can be calculated as: PI=(% monoenoic acids×0.025)+(% dienoic acids×1)+(% trienoic acids×2)+(% tetraenoic acids×4)+(pentaenoic acids×6)+(hexaenoic acid×8). Lipidomic measurements of cellular membrane composition are used to determine the peroxidizability index. Cell lines with low PI values (<50) have low sensitivity to ferroptosis-inducing perturbations (e.g., GPX4 inhibition, GSH depletion, addition of pro-oxidant compounds). Cells are more susceptible to undergoing ferroptosis with increasing membrane PI values.

Cells grown in vitro have fatty acid profiles unlike those of cells in vivo and lower PI levels. Vertebrate cells are unable to synthesize PUFAs de novo and rely on dietary sources for such molecules. Typical cell culture methods use media supplemented with serum (typically 10%, v/v), which is the only source of exogenous lipids and contains 1% of the PUFAs available to cells in the body. As a result, cells grown in culture have half the PUFA levels of cells in vivo and double the amount of monounsaturated fatty acids (MUFAs).

The ferroptosis sensitivity of cell lines can be modulated by inclusion of fatty acids in the culture medium. Saturated fatty acids (SFAs), monounsaturated fatty acids (MUFAs), and deuterated PUFAs protect cells from undergoing ferroptosis while the addition of PUFAs increases cell sensitivity to ferroptosis-inducing perturbations. Supplementation of cell culture media with exogenous PUFAs can simulate in vivo PUFA concentrations and induce membrane compositions with higher PI values. Modulatory profiling assays with fatty acid supplementation and ferroptosis inducers allows for the experimental determination of specific membrane PUFA content and PI values sufficient for ferroptosis for a given cell line. For example, the peroxidizability index (PI) of sarcoma and other cancer cells is greater than nonmalignant tissue due to preferential uptake of PUFAs. Many sarcomas preferentially uptake PUFAs and incorporate polyunsaturated fatty acyl chains into membrane lipids, resulting in higher membrane peroxidizability index values (PI>100) versus nonmalignant tissue (average PI=91). The difference in membrane peroxidizability provides a therapeutic window for ferroptosis induction to selectively target sarcoma cells versus nonmalignant tissue. The more peroxidizable membrane state is consistent with observations of higher levels of lipid peroxidative stress in primary bone and soft tissue sarcoma. Addition of exogenous PUFAs can increase oxidative stress in osteogenic sarcoma cells and exhibit selective cytotoxic effects.

Provided herein are methods of identifying a ferroptosis-sensitive cell in a mammalian tissue by the peroxidizability index (PI). In some embodiments, methods provided herein comprise administering to a mammal an effective amount of a ferroptosis-inducing agent, wherein a plurality of cells of the mammalian tissue have a PI greater than the PI in cells of normal or healthy tissue; and ferroptosis is induced in the plurality of cells. In some embodiments, the PI in the plurality of cells of the mammalian tissue is greater than a predetermined PI. In some embodiments, the predetermined PI is about 50, 60, 70, 80, 90, 100, 110, 120, 130, 140, or 150. In some embodiments, the predetermined PI is about 90. In some embodiments, the PI in the plurality of cells of the mammalian tissue is greater than the PI in cells of healthy or non-malignant tissue by about 1%-10%, 10%-20%, 20%-30%, 30%-40%, 40%-50%, 50%-60%, 60%-70%, 70%-80%, 80%-90%, or 90%-100%.

### (5) Mesenchymal Cell State

Therapy-resistant cells have three cellular and patient-derived signatures of high mesenchymal state. The first cellular signature is the expression of mesenchymal cell markers. Ferroptosis-sensitive cells exhibit a one or more marker of a mesenchymal cell state. Mesenchymal cell state markers that can be used to identify a ferroptosis-sensitive cell include but are not limited to: ZEB1, ACSL4, FADS2, PPARγ, Fsp1, SLC7A11, SLC3A2, and LPCAT3. The second cellular signature of a ferroptosis-sensitive cell is the reduced expression of endothelial cell markers as compared to normal cells. Non-limiting examples of endothelial cell markers include: vimentin, E-cadherin, and beta (β)-actin. The third cellular signature of a ferroptosis-sensitive cell is the sensitivity to GPX4 knockdown leading to cell death. GPX4 dependency is more pronounced in cancer cells adopting a therapy-resistant mesenchymal state as compared to normal mesenchymal cell lines. Methods of reducing or silencing GPX4 expression can be achieved, e.g., by CRISPR/Cas9, siRNA or shRNA, among others.

Provided herein are methods of identifying a ferroptosis-sensitive cell in a mammalian tissue by the expression of one or more mesenchymal cell state markers. In some embodiments, the methods provided herein comprise administering to a mammal an effective amount of a ferroptosis-inducing agent, wherein a plurality of cells of a mammalian tissue express one or more markers of a mesenchymal cell state; and ferroptosis is induced in the plurality of cells. In some embodiments, the expression of the mesenchymal cell marker in the plurality of cells of the mammalian tissue is greater than the expression of the mesenchymal cell marker in cells of healthy or non-malignant tissue by about 1%-10%, 10%-20%, 20%-30%, 30%-40%, 40%-50%, 50%-60%, 60%-70%, 70%-80%, 80%-90%, or 90%-100%.

### (6) Additional Morphological Characteristics Offerroptosis

Cells undergoing ferroptosis are characterized morphologically by the presence of smaller than normal mitochondria with condensed mitochondrial membrane densities, reduction or vanishing of mitochondria crista, and outer mitochondrial membrane rupture. Histology and immunoassays can be used to determine whether a tissue is cancerous, exhibits hyperplasia, or fibrosis, as well as identify ferroptosis-sensitive cells within a mammalian tissue. The cell membrane of cells in a ferroptotic state lack of rupture and blebbing of the plasma membrane normally associated with apoptosis. The nuclear size of ferroptotic cells is normal and lacks chromatin condensation.

In some embodiments, the methods provided herein comprise a step of obtaining a biological sample (e.g., blood sample or tissue biopsy) from a subject. In some embodiments, the methods provided herein further comprise fixing, processing, embedding, sectioning, and staining the biological sample for histological analysis. In some embodiments, the tissue comprises a histological abnormality. In some embodiments, the histological abnormality is determined by a tissue biopsy prior to or during the targeted, sustained administration of the ferroptosis-inducing agent to the tissue. In some embodiments, the histological abnormality is hyperplasia, vascularization/angiogenesis, or fibrosis. Hyperplasia is identified by an increased number of cells in a tissue as compared to a normal healthy tissue. Vascularization and angiogenesis are identified in a tissue sample by immunoassays for vascular markers, e.g., vascular endothelial growth factor (VEGF) and angiopoietin-2 (Ang2). Fibrosis is characterized by abnormal collagen deposits between cells identified in a tissue sample, e.g., by Masson's trichrome, Sirius red, or collagen staining.

### Cell-Death and Ferroptosis-Inducing Agents

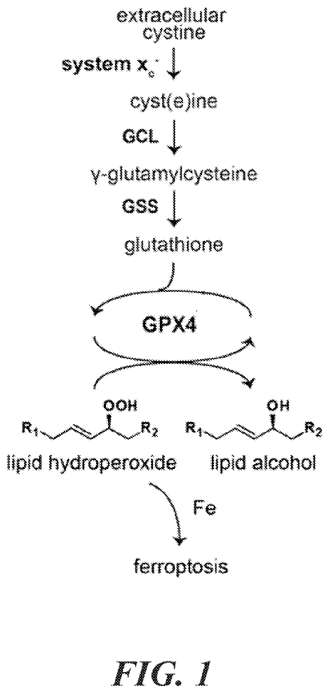

Provided herein are methods of inducing or modulating ferroptosis in vitro or in a tissue in a subject, wherein the methods comprise: (a) sustained administration of a therapeutic amount of a ferroptosis-inducing agent which can be a compound of Formula I, a compound of Formula II, a diastereomer or enantiomer of any of these, a pharmaceutically acceptable salt of any of these, a deuterated derivative of any of these, a composition containing any of these, or a pharmaceutical composition containing any of these, optionally in combination with a second therapeutic; (b) optionally contacting a tissue in vivo with an effective amount of an iron-dependent cell death agent for a duration of time; and/or (c) optionally contacting a mammalian tissue with a priming agent and then contacting the mammalian tissue in vivo with an effective amount of a ferroptosis-inducing agent for a duration of time, thereby inducing or modulating ferroptosis in a tissue in a subject. Exemplary targets in the ferroptosis pathway are provided in FIG. 1, and can comprise glutamate-cysteine ligase GCL or glutamate-cysteine ligase catalytic (GCLC) subunit.

Compounds herein, in some instances, can require substantially continuous or continuous administration and/or contact with a cell or a tissue at or above a threshold level to induce or modulate ferroptosis, or to modulate or inhibit or partially inhibit glutamate-cysteine ligase GCL or glutamate-cysteine ligase catalytic (GCLC) subunit, or to treat a disease or condition such as a cancer, a fibrosis, or an inflammatory disease.

Reference to a compound or an agent or a therapeutic and the like can include one or more of these, and can include or be a first compound or agent or therapeutic, a second compound or agent or therapeutic, a third compound or agent or therapeutic, a fourth compound or agent or therapeutic, or more.

### (1) Ferroptosis-Inducing Compounds, Agents and Iron-Dependent Cell Death Inducing Agents

In some embodiments, provided herein is a compound of Formula I:

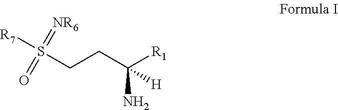

a diastereomer or enantiomer thereof, or a pharmaceutically acceptable salt of any of the foregoing, or a deuterated derivative of any of the forgoing,
wherein:
R₁ is:
   C(O)OH; or
   C(O)OX, wherein X is an organic cation, an inorganic cation, Na⁺, K⁺, Mg²⁺, Ca²⁺, Zn²⁺, or Mn²⁺; or

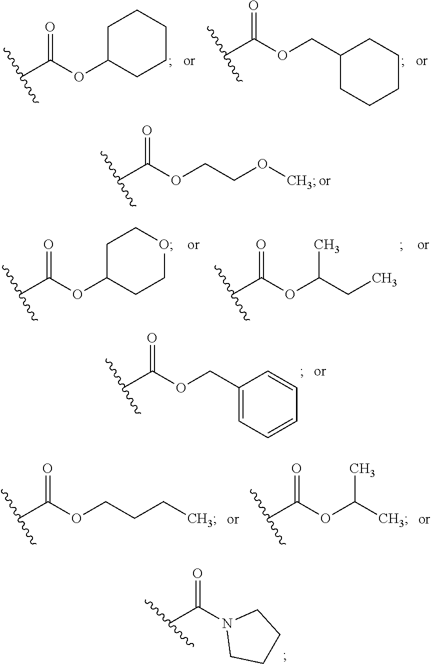

C(O)OR₃, wherein R₃ is a linear or branched alkyl, a cycloaklyl, a cyclic ether, or a linear or branched alkyl ether, wherein any of these is optionally and independently substituted;
   or
   R₃ is

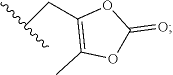 [SMILES: *Cc1oc(=O)oc1C]

or
R₃ is

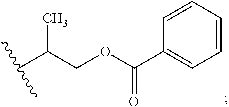 [SMILES: *C(C)COC(=O)c1ccccc1]

or
C(O)N(R₄R₅)
   wherein R₄ is H; or
   R₄ is a linear or branched chain alkyl, cycloalkyl, heterocycloalkyl, aryl, or heteroaryl; wherein any of these is optionally and independently substituted; and
   R₅ is H; or
   R₅ is a linear or branched chain alkyl, cycloalkyl, heterocycloalkyl, aryl, or heteroaryl; wherein any of these is optionally and independently substituted; or
   R₅ is S(O)₂alkyl; or
   R₅ is S(O)₂CF₃; or
   R₅ is S(O)₂NH₂; or
   R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
   R₅ is

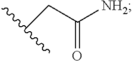 [SMILES: *CC(N)=O]

or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is

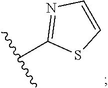 [SMILES: *c1nccs1]

or
R₅ is alkylaryl or benzyl; or
R₅ is

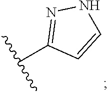 [SMILES: *c1cc[nH]n1]

or
R₅ is 2-pyridyl, 3-pyridyl; or 4-pyridyl; or
R₅ is

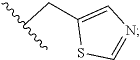 [SMILES: *Cc1cncs1]

or
R₅ is

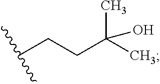 [SMILES: *CCC(C)(C)O]

or
R₅ is

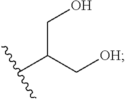 [SMILES: *C(CO)CO]

or
R₅ is

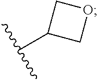 [SMILES: *C1COC1]

or
R₅ is

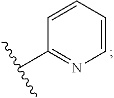 [SMILES: *c1ccccn1]

or
CN; or

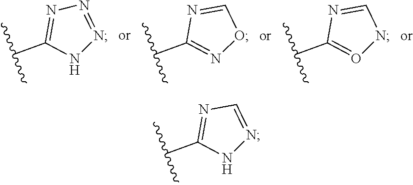

and wherein
R₂ is: NH₂, NHC(O)OMe, or NHMe; and
R₆ is: H, C(O)Me, or P(O)(OH)₂; and
R₇ is: C₁-C₃ or C₅-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl, any of the foregoing can be independently and optionally substituted, wherein when the linear C₃-alkyl is substituted on a terminal carbon atom by a methyl group, the linear C₃-alkyl substituted on a terminal carbon atom by the methyl group contains a further substitution; wherein when the C₂-alkyl is substituted on a terminal carbon atom by an ethyl group, the C₂-alkyl substituted on a terminal carbon atom by an ethyl group contains a further substation; wherein when the C₁-alkyl is substituted on a terminal carbon atom by an n-propyl group, the C₁-alkyl substituted on a terminal carbon atom by an n-propyl group contains a further substation; or
R₇ is:

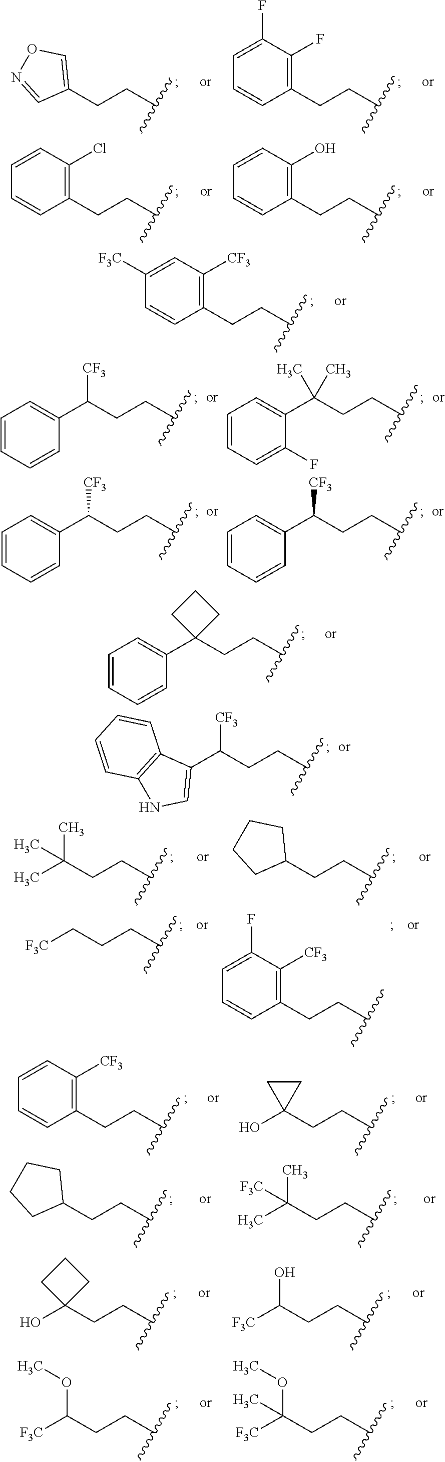

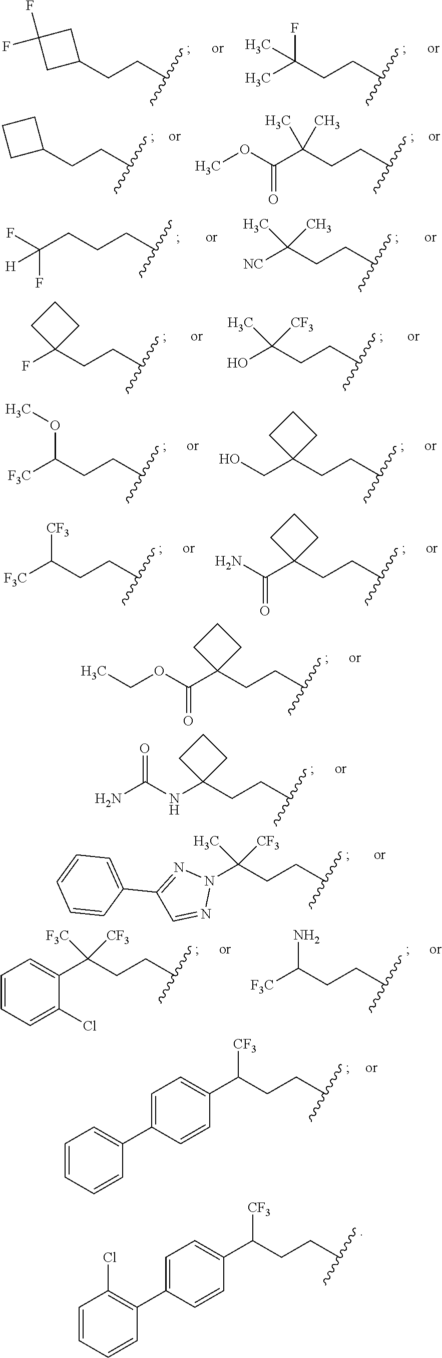

In some instances, in the compound of Formula I, the diastereomer or enantiomer thereof, the pharmaceutically acceptable salt of any of the foregoing, or the deuterated derivative of any of the foregoing, R1 is:
C(O)OH; or
C(O)OR₃,
where R₃ is a linear or branched chain alkyl, C₁-C₁₀ linear or branched chain alkyl, methyl, ethyl, n-propyl, iso-propyl, iso-butyl, sec-butyl, n-butyl, tert-butyl, pentyl, hexyl, heptyl, octyl, nonyl, cycloalkyl, C₁-C₁₀ cycloalkyl, cyclopropyl, cyclobutyl, cyclopentyl, cyclohexyl, cycloheptyl, alkylcycloalkyl, alkylcyclohexyl, methylcyclopropyl, methylcycobutyl, methylcyclopentyl, methylcyclohexyl, methylcycloheptyl, an alkyl ether, 2-methoxyethyl, 3-methoxypropyl, 4-methoxybutyl, 5-methoxypropyl, cyclicalkylether, tetrahydropyranyl, 2-tetrahydropyranyl, 3-tetrahydropyranyl, 4-tetrahydropyranyl, tetrahydrofuranyl, 2-tetrahydrofuranyl, 2-tetrahydrofuranyl, alkylaryl, benzyl;
and any of these is optionally and independently substituted with one or more C₁-C₁₀ alkyl, one or more halogens, one or more fluoro, one or more chloro, one or more deuterium, bromo, one or more iodo, aryl, C₆ aryl, C₁₀ aryl, heteroaryl, a C₁-C₇ alkylcycloaklyl, an unsubstituted tetrahydropyranyl, 2-tetrahydropyranyl, 3-tetrahydropyranyl, or 4-tetrahydropyranyl or any combination thereof.

In some instances, in the compound of Formula I, the diastereomer or enantiomer thereof, the pharmaceutically acceptable salt of any of the foregoing, or the deuterated derivative of any of the foregoing
R₁ is C(O)N(R₄R₅)
   where R₅ is:
   C₁-C₁₀ a linear or branched chain alkyl, methyl, ethyl, propyl, or butyl; any of which are optionally or independently substituted by one or more deuterium, C₁-C₁₀ linear or branched chain alkyl, one or more halo, one or more fluoro, one or more chloro, one or more iodo, one or more or any combination thereof.

In some instances, in the compound of Formula I, the diastereomer or enantiomer thereof, the pharmaceutically acceptable salt of any of the foregoing, or the deuterated derivative of any of the foregoing,
R₁ is C(O)N(R₄R₅) and
   wherein R₅ is: heteroaryl, 2-pyridyl, 3-pyridyl, or 4-pyridyl; or R₅ is S(O)₂Me.

In some instances, in the compound of Formula I, the diastereomer or enantiomer thereof, the pharmaceutically acceptable salt of any of the foregoing, or the deuterated derivative of any of the foregoing
R₇ is: C₁-C₃ or C₅-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl; where any of foregoing can be independently and optionally substituted by one or more of a substituent that can be: deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an amino, a carboxylic acid or pharmaceutically acceptable salt thereof, an amide, a carbamate, a urea, an ester, an alkoxy, a methoxy, an ethoxy, a trifluoro methoxy, an ether, a cyclic ether, an C₁-C₇ alkyl ether, a C₁-C₇ cyclic alkyl ether, a trihalomethyl, a trifluoromethyl, an aryl, a heteroaryl, a fused aryl, a bi-aryl, a fused aryl-heteroaryl, a fused di-aryl, a fused aryl-heteroaryl, a 5-membered heteroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal; a ketal; or any combination of these;
where:
when the linear C₃-alkyl is substituted on a terminal carbon atom by a methyl group substituent, the linear C₃-alkyl substituted on a terminal carbon atom by the methyl group substituent contains a further substitution;
when the C₂-alkyl is substituted on a terminal carbon atom by an ethyl group, the C₂-alkyl substituted on a terminal carbon atom by an ethyl group contains a further substation;
when the C₁-alkyl is substituted on a terminal carbon atom by an n-propyl group, the C₁-alkyl substituted on a terminal carbon atom by an n-propyl group contains a further substation; and
where the C₁-C₁₀ linear alkyl substituent, the methyl substituent, the ethyl substituent, the C₁-C₁₀ branched chain alkyl substituent, the hydroxyl substituent, the amino substituent, the carboxylic acid or pharmaceutically acceptable salt thereof substituent, the amide substituent, the carbamate substituent, the urea substituent, the ester substituent, the alkoxy substituent, the methoxy substituent, the ethoxy substituent, the ether substituent, the cyclic ether substituent, the C₁-C₇ alkyl ether substituent, the C₁-C₇ cyclic ether substituent, the aryl substituent, the heteroaryl substituent, the fused aryl substituent, the bi-aryl substituent, the fused aryl-heteroaryl substituent, the fused di-aryl substituent, the fused aryl-heteroaryl substituent, the 5-membered heteroaryl substituent, the 6-membered heteroaryl substituent, the naphthyl substituent, the cycloalkyl substituent, the cyclopropyl substituent, the cyclobutyl substituent, the cyclopentyl substituent, the cyclohexyl substituent, the cycloheptyl substituent, the tert-butyl substituent, the bicyclic aliphatic substituent, the tricyclic aliphatic substituent, the adamantly substituent, or any combination of these can be independently and optionally substituted by one or more of:
a C₁-C₁₀ linear chain alkyl, a methyl, an ethyl, a C₁-C₁₀ branched chain alkyl, a halogen, a fluoro, a chloro, a bromo, an iodo, a hydroxyl, an alkoxy, a methoxy, an ethoxy, a carbamate, a urea, an amide, an ester, an amine, a trifluoro methoxy, an ether, an C₁-C₇ alkyl ether, a C₁-C₇ cyclic ether, a trihalomethyl, a trifluoromethyl, an aryl, a heteroaryl, a 5-membered hereroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal, a ketal, or any combination of these.

Also provided is a compound of Formula I:

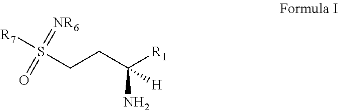

a diastereomer or enantiomer thereof, or a pharmaceutically acceptable salt of any of the foregoing, or a deuterated derivative of any of the forgoing,
wherein:
R₁ is:
   C(O)OH; or
   C(O)OX, wherein X is an organic cation, an inorganic cation, Na⁺, K⁺, Mg²⁺, Ca²⁺, Zn²⁺, or Mn²⁺; or

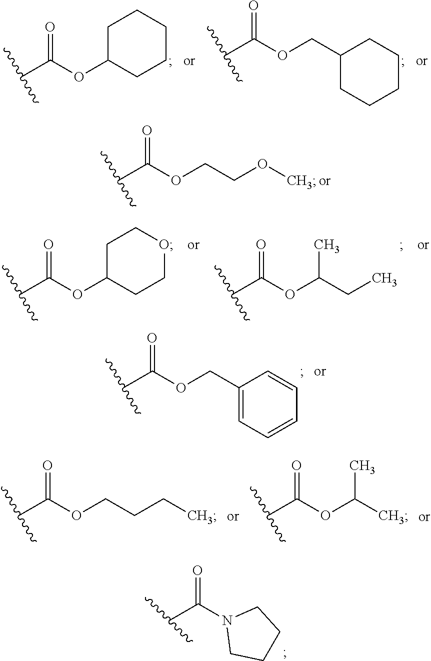

or
C(O)OR₃, wherein R₃ is a linear or branched alkyl, a cycloaklyl, a cyclic ether, or a linear or branched alkyl ether, wherein any of these is optionally and independently substituted;
   or
   R₃ is

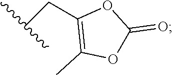 [SMILES: *Cc1oc(=O)oc1C]

or
R₃ is

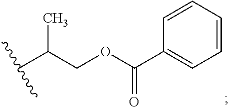 [SMILES: *C(C)COC(=O)c1ccccc1]

or
C(O)N(R₄R₅)
   wherein R₄ is H; or
   R₄ is a linear or branched chain alkyl, cycloalkyl, heterocycloalkyl, aryl, or heteroaryl; wherein any of these is optionally and independently substituted; and
   R₅ is H; or
   R₅ is a linear or branched chain alkyl, cycloalkyl, heterocycloalkyl, aryl, or heteroaryl; wherein any of these is optionally and independently substituted; or
   R₅ is S(O)₂alkyl; or
   R₅ is S(O)₂CF₃; or
   R₅ is S(O)₂NH₂; or
   R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
   R₅ is

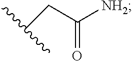 [SMILES: *CC(N)=O]

or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is

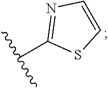 [SMILES: *c1nccs1]

or
R₅ is alkylaryl or benzyl; or
R₅ is

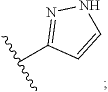 [SMILES: *c1cc[nH]n1]

or
R₅ is 2-pyridyl, 3-pyridyl; or 4-pyridyl; or
R₅ is

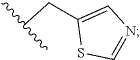 [SMILES: *Cc1cncs1]

or
R₅ is

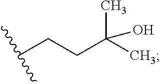 [SMILES: *CCC(C)(C)O]

or
R₅ is

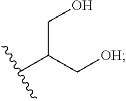 [SMILES: *C(CO)CO]

or
R₅ is

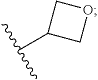 [SMILES: *C1COC1]

or
R₅ is

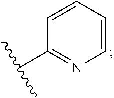 [SMILES: *c1ccccn1]

or
CN; or

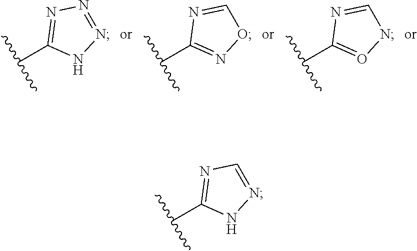

and
R₂ is: NH₂, NHC(O)OMe, or NHMe; and
R₆ is: H, C(O)Me, or P(O)(OH)₂; and
R₇ is: linear or branched chain: alkyl, alkenyl, or alkynyl, any of which can optionally and independently be substituted; or
R₇ is:

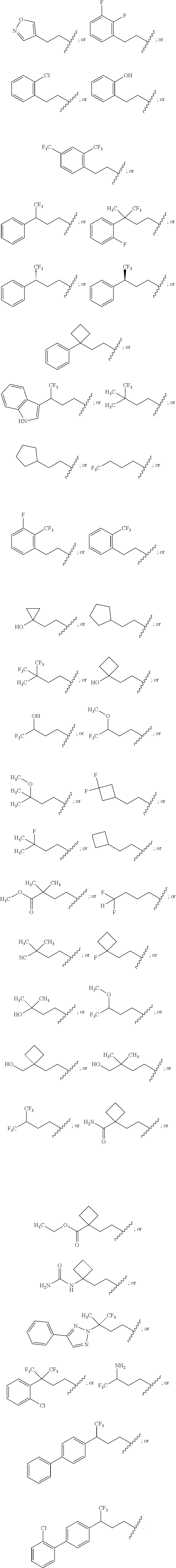

and wherein the compound of Formula I is not buthionine sulfoximine (BSO) or a salt of BSO.

Also provided herein is a compound of Formula II

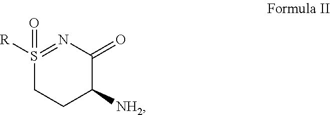

a diastereomer or an enantiomer of the compound of Formula II, or a pharmaceutically acceptable salt of any of the foregoing, or a deuterated derivative of any of the foregoing; wherein in the compound of Formula II, R is a: C₁, C₂, C₃, C₄, C₅, C₆, C₇, C₈, C₉, or C₁₀ linear or branched chain alkyl, optionally and independently substituted by a substituent selected from the group consisting of: a deuterium, a halogen, a fluorine, a chlorine, a C₁-C₁₀ linear or branched chain alkyl, a methyl, an ethyl, a propyl, an iso-propyl, a butyl, an isobutyl, a sec-butyl, a tert-butyl, and any combination of these.

Also provided herein is a pharmaceutical composition comprising the compound of Formula I, the compound of Formula II, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. The pharmaceutical composition can be in unit dose form. Additionally, the pharmaceutical composition can comprise an additional active agent or pharmaceutically acceptable salt thereof or prodrug thereof. Further, the pharmaceutical composition can be in the form of a powder, a tablet, a capsule, a liquid, or a gel. In some embodiments, in the pharmaceutical composition, the compound of Formula I, the compound of Formula II, the compound; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg. In some embodiments, the additional active agent or pharmaceutically acceptable salt thereof or prodrug thereof can be independently present in the pharmaceutical composition in an amount ranging from about 0.001 mg to about 25,000 mg.

In some embodiments is provided a kit comprising a compound therein, a diastereomer thereof, an enantiomer thereof, a pharmaceutically acceptable salt thereof, or a deuterated derivative thereof, or a pharmaceutically composition therein, and a container. In some embodiments are pharmaceutical compositions described herein and a container. In some embodiments, the container is a syringe. In some embodiments, the container is an intravenous (IV) bag. In some embodiments, the container is disposable. In some embodiments, the container is recyclable. In some embodiments, the container is a single use container. In some embodiments, the container is resealable.

In some embodiments is provided a method of treating a disease or condition in a subject. In some embodiments, the disease or condition is a cancer. In some embodiments, the method comprises administering a therapeutically effective amount of the pharmaceutical composition herein to the subject, who can be a subject in need thereof, thereby treating the disease or the condition, which can be a cancer. In some embodiments is provided a method of treating a disease or condition in a subject, who can be subject in need thereof, the method comprising administering the compound of Formula I, the compound of Formula II, the diastereomer or the enantiomer of any of the foregoing, or the pharmaceutically acceptable salt of any of the foregoing, or the deuterated derivative of any of the foregoing; in a therapeutically effective amount to the subject, thereby treating the disease or condition, which can be a cancer. In some embodiments, the administering is selected from the group consisting of: oral, an injection; subcutaneous, intra-tumoral; systemic, local, intravenous, intraperitoneal, intramuscular, and any combination thereof. In some embodiments, the subject can be a mammal. In some embodiments, the subject can be a human. In some embodiments, the subject can be a male. In some embodiments, the subject can be a female. In some embodiments is provided a method of treating a disease or condition in a subject, who can be subject in need thereof, the method comprising administering the compound of Formula XVIII, Formula XIX, Formula XX, the diastereomer or the enantiomer of any of the foregoing, or the pharmaceutically acceptable salt of any of the foregoing, or the deuterated derivative of any of the foregoing; in a therapeutically effective amount to the subject, thereby treating the disease or condition, which can be a cancer. In some embodiments, the administering is selected from the group consisting of: oral, an injection; subcutaneous, intra-tumoral; systemic, local, intravenous, intraperitoneal, intramuscular, and any combination thereof. In some embodiments, the subject can be a mammal. In some embodiments, the subject can be a human. In some embodiments, the subject can be a male. In some embodiments, the subject can be a female.

In some embodiments is provided a method of modulating ferroptosis in a tissue, which can be in a subject, which can be a subject in need thereof, the method comprising contacting, for example directly or indirectly, optionally in a sustained manner, the tissue with a pharmaceutical composition herein in an amount effective to modulate the ferroptosis in the tissue. In some embodiments, the subject can be a human. In some embodiments, the subject can be a male. In some embodiments, the subject can be a female.

In some embodiments, in a method herein, the administering or the contacting can be: as needed, once per day, twice per day, three times per day, once per week, once per two weeks, once per three weeks, once per month, once every six months, once per year, or for life. In some embodiments, an effective or a therapeutically effective amount can range from about 0.001 mg to about 25,000 mg of a compound herein, an enantiomer or a diastereomer thereof, a pharmaceutically acceptable salt of an of these, or a deuterated derivative of any of these, or of a pharmaceutical composition herein, which can optionally be in unit dose form.

In some embodiments, also are provided methods of making and testing compounds of Formula I and Formula II, enantiomers and diastereomers of any of these, salts and pharmaceutically acceptable salts of any of these, and deuterated derivatives of any of these. In some embodiments, also are provided methods of making and testing compounds of Formula XVIII, Formula XIX, Formula XX, enantiomers and diastereomers of any of these, salts and pharmaceutically acceptable salts of any of these, and deuterated derivatives of any of these.

The methods provided herein comprise administering to a cell, tissue, or subject an agent, compound, or therapeutic that modulates cell death. In some embodiments, the administering induces cell death. In some embodiments, the administering inhibits or rescues a cell from cell death. In some embodiments, the administering modulates ferroptosis. In some embodiments, the administering induces ferroptosis in vivo. In some embodiments, the administering inhibits ferroptosis in vivo. In some embodiments, the agent is a ferroptosis-inducing agent. In some embodiments, the agent is an iron-dependent cell death inducing agent. Agents useful in the induction of ferroptosis in vivo and for the treatment of a disease or disorder are discussed in further detail below.

In some embodiments, the agent is an inhibitor of glutamate-cysteine ligase (GCL). Glutamate-cysteine ligase (GCL), a central node in the ferroptosis pathway, has been overlooked as a target. Loss of GCL activity induces ferroptosis in sensitive cells and kills only the most ferroptosis-sensitive cells. Representative human GCL cDNA and human GCL protein sequences are publicly available from the National Center for Biotechnology Information (NCBI). Human glutamate-cysteine ligase catalytic subunit isoform b (NM_001197115.2 and NP_001184044.1, which lacks an in-frame exon in the 5′ coding region, compared to variant 1. This results in a shorter protein (isoform b), compared to isoform a), and glutamate-cysteine ligase catalytic subunit isoform a (NM_001498.4 and NP_001489.1, which represents the longer transcript and encodes the longer isoform (a)).

In some embodiments, the agent is an inhibitor of glutamate-cysteine ligase catalytic subunit (GCLC).

In some embodiments, molecules herein, enantiomers, diastereomers, mixtures herein, hydrates thereof, deuterated analogs thereof, salts thereof, pharmaceutically acceptable salts thereof, compositions comprising any of these, or pharmaceutical compositions comprising any of these, can be used to treat a cancer or neoplastic condition. In some embodiments, the cancer or neoplastic condition can be a skin cancer, a sarcoma, a melanoma, a carcinoma, a mesenchymal cancer, a breast cancer, a prostate cancer, a cervical cancer, or an ovarian cancer, a kidney cancer, a renal cancer, a liver cancer, a liver carcinoma, renal carcinoma, non-clear cell renal carcinoma, or clear cell renal carcinoma. In some embodiments, molecules herein, enantiomers, diastereomers, mixtures herein, hydrates thereof, deuterated analogs thereof, salts thereof, pharmaceutically acceptable salts thereof, compositions comprising any of these, or pharmaceutical compositions comprising any of these, can be used to treat a SWI/SNF complex-deficient cancer. In some embodiments, the SWI/SNF complex-deficient cancer can be a skin cancer, a sarcoma, a melanoma, a mesenchymal cancer, a breast cancer, a prostate cancer, a cervical cancer, or an ovarian cancer, a kidney cancer, a renal cancer, a liver cancer, a carcinoma, a liver carcinoma, clear cell renal carcinoma, or non-clear cell renal carcinoma.

In some embodiments, the agent is a statin. Exemplary statins include but are not limited to: atorvastatin, cerivastatin, fluvastatin, lovastatin, mevastatin, pitavastatin, pravastatin, rosuvastatin and simvastatin.

In some embodiments, an agent that induces ferroptosis in a tissue is selected from Table 1. Exemplary ferroptosis-inducing agents are provided in Table 1 along with their formula, chemical identifiers, and respective target and/or mechanism of action.

TABLE 1
Ferroptosis-inducing agents

| | | | Target(s)/ |
|---|---|---|---|
| | Formula/ | | Mechanism of |
| Name | CAS | Structure | Action |
| (1S,3R)- RSL3 | C₂₃H₂₁ClN₂O₅ Cas: 1219810- 16-8 | 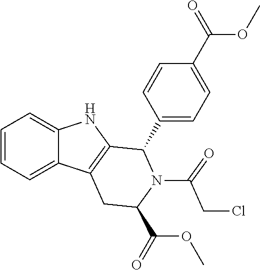 [SMILES: COC(=O)c1ccc([C@H]2c3[nH]c4ccccc4c3C[C@H](C(=O)OC)N2C(=O)CCl)cc1] | Ferroptosis inducer by GPX4 inhibition |
| altretamine | C₉H₁₈N₆ Cas: 645- 05-6 | 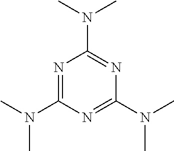 | Ferroptosis inducer by GPX4 inhibition |
| auranofin | C₂₀H₃₄AuO₉PS Cas: 34031-32-8 | 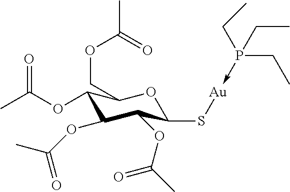 | Multiple modes of action including inhibition of thioredoxin reductase (TXNRD) |
| brusatol | C₂₆H₃₂O₁₁ Cas: 14907-98-3 | 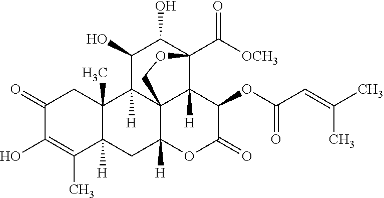 | Ferroptosis inducer by NRF2 inhibition. NRF2 is a transcriptional regulator of GPX4 protein content |
| Chlorido[N, N′- disalicylidene- 1,2- phenylenedia- mine]iron(III) | C₂₀H₁₄ClFeN₂O₂ Cas: 39916-28-4 | 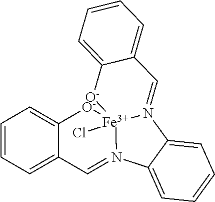 | Ferroptosis inducer by generating lipid- based reactive oxygen species (ROS) |
| CIL56 | C₂₃H₂₇N₃O₅S₂ Cas: 300802-28-2 | 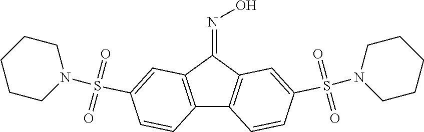 [SMILES: O=S(=O)(c1ccc2c(c1)C(=NO)c1cc(S(=O)(=O)N3CCCCC3)ccc1-2)N1CCCCC1] | Ferroptosis inducer by generating lipid- based reactive oxygen species (ROS) |
| Dihydroisota | C₁₈H₁₄O₃ | | Ferroptosis |
| ns-hinone I | Cas: | | inducer by |
| | 20958-18-3 | | increasing lipid |
| | | | peroxidation and |
| | | | GPX4 inhibition |
| erastin | C₃₀H₃₁ClN₄O₄ Cas: 571203-78-6 | 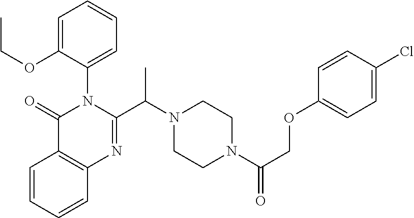 [SMILES: CCOc1ccccc1-n1c(C(C)N2CCN(C(=O)COc3ccc(Cl)cc3)CC2)nc2ccccc2c1=O] | Ferroptosis inducer by inhibition of cystine uptake by the system xc- cystine-glutamate transporter |
| Erastin-like- PE | C₃₅H₄₁ClN₆O₄ | 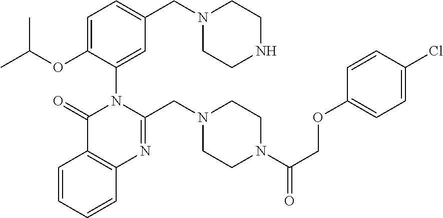 [SMILES: CC(C)Oc1ccc(CN2CCNCC2)cc1-n1c(CN2CCN(C(=O)COc3ccc(Cl)cc3)CC2)nc2ccccc2c1=O] | Ferroptosis inducer by inhibition of cystine uptake by the system xc- cystine-glutamate transporter |
| Erastin-like- IKE | C₃₅H₃₅ClN₆O₅ | 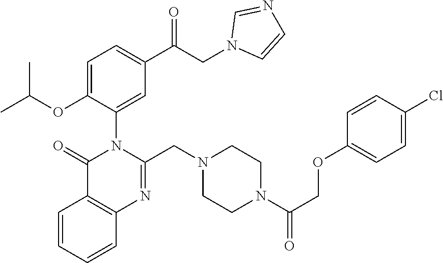 [SMILES: CC(C)Oc1ccc(C(=O)Cn2ccnc2)cc1-n1c(CN2CCN(C(=O)COc3ccc(Cl)cc3)CC2)nc2ccccc2c1=O] | Ferroptosis inducer by inhibition of cystine uptake by the system xc- cystine-glutamate transporter |
| | | Formula III: | |
| erastin- related | | 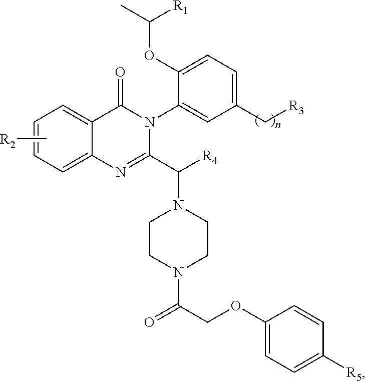 | Ferroptosis inducer by inhibition of cystine uptake by the system xc- cystine-glutamate transporter |
| | | or | |
| | | an enantiomer, optical isomer, diastereomer, | |
| | | N-oxide, crystalline form, hydrate, or | |
| | | pharmaceutically acceptable salt thereof, | |
| | | wherein in the compound of Formula III: | |
| | | R₁ is selected from the group consisting of H, | |
| | | C₁₋₄ alkyl, C₁₋₄ alkoxy, hydroxy, and halogen; | |
| | | R₂ is selected from the group consisting of | |
| | | H, halo, and C1-4 alkyl; R₃ is selected from | |
| | | the group consisting of H, C₁₋₄ alkyl, C₁₋₄ | |
| | | alkoxy, 5-7 membered heterocycloalkyl, and | |
| | | 5-6 membered heteroaryl; R₄ is selected from | |
| | | the group consisting of H and C₁₋₄ alkyl; R₅ | |
| | | is halo; | |
| | | 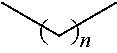 | |
| | | is optionally substituted with ═O; and | |
| | | n is an integer from 0-4. | |
| | | Formula IV: | |
| erastin- related | | 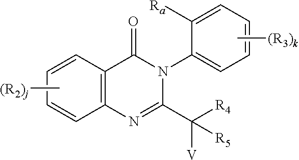 | Ferroptosis inducer by inhibition of cystine uptake by the system xc- cystine-glutamate transporter |
| | | or an | |
| | | enantiomer, optical isomer, diastereomer, N- | |
| | | oxide, crystalline form, hydrate, or | |
| | | pharmaceutically acceptable salt thereof, | |
| | | wherein in the compound of Formula IV: | |
| | | Rₐ is a halogen, substituted or unsubstituted | |
| | | alkyl, substituted or unsubstituted alkenyl, | |
| | | substituted or unsubstituted alkynyl, | |
| | | substituted or unsubstituted aryl-O—, | |
| | | substituted or unsubstituted alkyl-O—, | |
| | | substituted or unsubstituted alkenyl-O— or | |
| | | substituted or unsubstituted alkynyl-O—, | |
| | | where alkyl, alkenyl and alkynyl are | |
| | | optionally interrupted by NR, O or S(O)ₙ; | |
| | | each R₂ is independently selected from the | |
| | | group consisting of halogen, substituted or | |
| | | unsubstituted alkyl, substituted or | |
| | | unsubstituted aryl, substituted or | |
| | | unsubstituted non-aromatic heterocyclic, | |
| | | —CN, —COOR′, —CON(R)₂, —NRC(O)R, | |
| | | —SO₂N(R)₂, —N(R)₂, —NO₂, —OH and | |
| | | —OR′; each R₃ is independently selected from | |
| | | the group consisting of halogen, substituted | |
| | | or unsubstituted alkyl, substituted or | |
| | | unsubstituted aryl, substituted or | |
| | | unsubstituted non-aromatic heterocyclic, | |
| | | —(CO)R, —CN, —COOR′, —CON(R)₂, | |
| | | —NRC(O)R, —SO₂N(R)₂, —N(R)₂, —NO₂, | |
| | | —OH and —OR′; R₄ and R₅ are | |
| | | independently selected from the group | |
| | | consisting of —H, substituted or | |
| | | unsubstituted alkyl, substituted or | |
| | | unsubstituted alkenyl, substituted or | |
| | | unsubstituted alkynyl, substituted or | |
| | | unsubstituted non-aromatic heterocyclic and | |
| | | substituted or unsubstituted aryl, where | |
| | | alkyl, alkenyl and alkynyl are optionally | |
| | | interrupted by NR, O or S(O)ₙ; or R₄ and R₅ | |
| | | taken together form a carbocyclic or | |
| | | heterocyclic group; V is | |
| | | 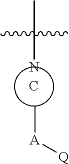 | |
| | | wherein Ring C is a substituted | |
| | | or unsubstituted heterocyclic aromatic or | |
| | | non-aromatic ring; A is NR or O; or A is a | |
| | | covalent bond; L is a substituted or | |
| | | unsubstituted hydrocarbyl group optionally | |
| | | interrupted by one or more heteroatoms | |
| | | selected from N, O and S; Q is selected from | |
| | | the group consisting of —R, —C(O)R′, | |
| | | —C(O)N(R)2, —C(O)OR′, and —S(O)2R′; | |
| | | each R is independently —H, alkyl, alkenyl, | |
| | | alkynyl, aryl, or non-aromatic heterocyclic, | |
| | | wherein said alkyl, alkenyl, alkynyl, aryl, or | |
| | | non-aromatic heterocyclic groups are | |
| | | substituted or unsubstituted; each R′ is | |
| | | independently an alkyl, alkenyl, alkynyl | |
| | | group, non-aromatic heterocyclic or aryl | |
| | | group, wherein said alkyl, alkenyl, alkynyl, | |
| | | non-aromatic heterocyclic or aryl groups are | |
| | | substituted or unsubstituted; j is an integer | |
| | | from 0 to 4; k is an integer from 0 to 4, | |
| | | provided that at least one of j and k is an | |
| | | integer from 1 to 4; and each n is | |
| | | independently 0, 1 or 2. | |
| | | Formula V: | |
| erastin- related | | 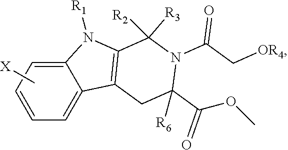 | Ferroptosis inducer by inhibition of cystine uptake by the system xc- cystine-glutamate transporter |
| | | or an enantiomer, optical isomer, | |
| | | diastereomer, N-oxide, crystalline form, | |
| | | hydrate, or pharmaceutically acceptable salt | |
| | | thereof, wherein in the compound of | |
| | | Formula V: R₁, R₂, R₃, and R₆ are | |
| | | independently selected from H, C₁₋₈ alkyl, | |
| | | C₁₋₈ alkoxy, C₁₋₈ aralkyl, 3- to 8-membered | |
| | | carbocyclic, 3- to 8-membered heterocyclic, | |
| | | 3- to 8-membered aryl, or 3- to 8-membered | |
| | | heteroaryl, acyl, alkylsulfonyl, and | |
| | | arylsulfonyl, wherein each alkyl, alkoxy, | |
| | | aralkyl, carbocyclic, heterocyclic, aryl, | |
| | | heteroaryl, acyl, alkylsulfonyl, and | |
| | | arylsulfonyl is optionally substituted with at | |
| | | least one substituent; R₄ and R₅ are | |
| | | independently selected from H1 C₁₋₈ alkyl, | |
| | | C₁₋₈ alkoxy, 3- to 8-membered carbocyclic, | |
| | | 3- to 8-membered heterocyclic, 3- to 8- | |
| | | membered aryl, or 3-to 8-membered | |
| | | heteroaryl, carboxylate, ester, amide, | |
| | | carbohydrate, amino acid, acyl, alkoxy- | |
| | | substituted acyl, alditol, NR⁷R⁸, | |
| | | OC(R⁷)₂COOH, SC(R⁷)₂COOH, | |
| | | NHCHR⁷COOH, COR⁸, CO₂R⁸, sulfate, | |
| | | sulfonamide, sulfoxide, sulfonate, sulfone, | |
| | | thioalkyl, thioester, and thioether, wherein | |
| | | each alkyl, alkoxy, carbocyclic, heterocyclic, | |
| | | aryl, heteroaryl, carboxylate, ester, amide, | |
| | | carbohydrate, amino acid, acyl, alkoxy- | |
| | | substituted acyl, alditol, NR⁷R⁸, | |
| | | OC(R⁷)₂COOH, SC(R⁷)₂COOH, | |
| | | NHCHR⁷COOH, COR⁸, CO₂R⁸, sulfate, | |
| | | sulfonamide, sulfoxide, sulfonate, sulfone, | |
| | | thioalkyl, thioester, and thioether is | |
| | | optionally substituted with at least one | |
| | | substituent; R₇ is selected from H, C₁₋₈ alkyl, | |
| | | carbocycle, aryl, heteroaryl, heterocycle, | |
| | | alkylaryl, alkylheteroaryl, and | |
| | | alkylheterocycle, wherein each alkyl, | |
| | | carbocycle, aryl, heteroaryl, heterocycle, | |
| | | alkylaryl, alkylheteroaryl, and | |
| | | alkylheterocycle may be optionally | |
| | | substituted with at least one substituent; R₈ is | |
| | | selected from H, C₁₋₈ alkyl, C₁₋₈ alkenyl, C₁₋₈ | |
| | | alkynyl, aryl, carbocycle, heteroaryl, | |
| | | heterocycle, alkylaryl, alkylheteroaryl, | |
| | | alkylheterocycle, and heteroaromatic, | |
| | | wherein each alkyl, alkenyl, alkynyl, aryl, | |
| | | carbocycle, heteroaryl, heterocycle, | |
| | | alkylaryl, alkylheteroaryl, alkylheterocycle, | |
| | | and heteroaromatic may be optionally | |
| | | substituted with at least one substituent; and | |
| | | X is 0-4 substituents on the ring to which it is | |
| | | attached. | |
| | | Formula VI: | |
| erastin- related | | 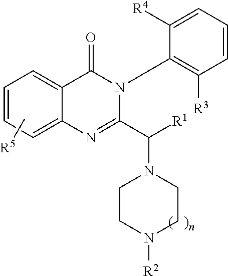 | Ferroptosis inducer by inhibition of cystine uptake by the system xc- cystine-glutamate transporter |
| | | or an | |
| | | enantiomer, optical isomer, diastereomer, N- | |
| | | oxide, crystalline form, hydrate, or | |
| | | pharmaceutically acceptable salt thereof, | |
| | | wherein in the compound of Formula VI | |
| | | R₁ is selected from H and C₁₋₈ alkyl; R₂ is | |
| | | selected from H and C₁₋₈ alkyl; R₃ is selected | |
| | | from halogen, C₁₋₈alkoxy and C₁₋₈ alkyl; R4 | |
| | | is selected from H, halogen, C₁₋₈ alkoxy and | |
| | | C₁₋₈ alkyl; R₅ is selected from H, halogen and | |
| | | nitro; and n is 1 or 2. | |
| | | Formula VII: | |
| erastin- related | | 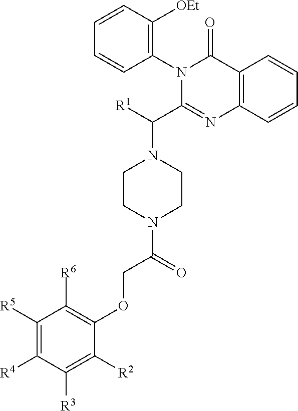 | Ferroptosis inducer by inhibition of cystine uptake by the system xc- cystine-glutamate transporter |
| | | wherein in the compound of Formula VII, R₁ | |
| | | is selected from H, C₁₋₈alkyl, C₁₋₈ alkoxy, 3- | |
| | | to 8-membered carbocyclic or heterocyclic, | |
| | | aryl, heteroaryl, C₁₋₄ aralkyl, residues of | |
| | | glycolic acid, ethylene glycol/propylene | |
| | | glycol copolymers, carboxylate, ester, amide, | |
| | | carbohydrate, amino acid, alditol, | |
| | | OC(R⁷)₂COOH, SC(R⁷)₂COOH, | |
| | | NHCHR⁷COOH, COR⁸, CO²R⁸, sulfate, | |
| | | sulfonamide, sulfoxide, sulfonate, sulfone, | |
| | | thioalkyl, thioester, and thioether; R₂, R₃, R₄, | |
| | | R₅, and R₆ are independently selected from | |
| | | H, halo, C₁₋₄alkyl, C₁₋₄ alkylamino, acyl, and | |
| | | alkylsulfonyl; R₇ is selected from H, C₁₋₈ | |
| | | alkyl, optionally substituted carbocycle, | |
| | | aryl, heteroaryl, heterocycle, alkylaryl, | |
| | | alkylheteroaryl, and alkylheterocycle; and R₈ | |
| | | is selected from optionally substituted C₁₋₈ | |
| | | alkyl, C₁₋₈alkenyl, C₁₋₈alkynyl, aryl, | |
| | | carbocycle, heteroaryl, heterocycle, | |
| | | alkylaryl, alkylheteroaryl, alkylheterocycle, | |
| | | and heteroaromatic. | |
| erastin-like- AM | | 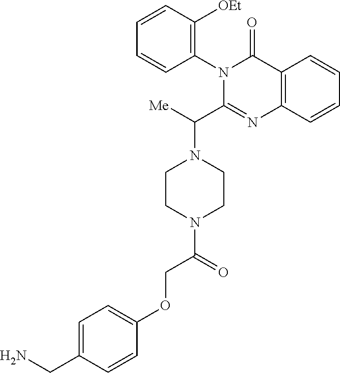 [SMILES: CCOc1ccccc1-n1c(C(C)N2CCN(C(=O)COc3ccc(CN)cc3)CC2)nc2ccccc2c1=O] | Ferroptosis inducer by inhibition of cystine uptake by the system xc- cystine-glutamate transporter |
| erastin-like- PHTL | | 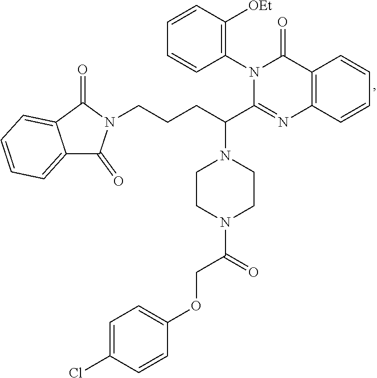 [SMILES: CCOc1ccccc1-n1c(C(CCCN2C(=O)c3ccccc3C2=O)N2CCN(C(=O)COc3ccc(Cl)cc3)CC2)nc2ccccc2c1=O] | Ferroptosis inducer by inhibition of cystine uptake by the system xc- cystine-glutamate transporter |
| erastin-like- H2 | | 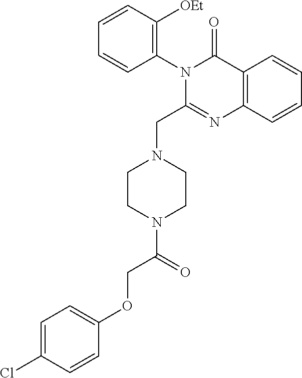 [SMILES: CCOc1ccccc1-n1c(CN2CCN(C(=O)COc3ccc(Cl)cc3)CC2)nc2ccccc2c1=O] | Ferroptosis inducer by inhibition of cystine uptake by the system xc- cystine-glutamate transporter |
| | | Formula VIII: | |
| erastin- related (Formula VI) | | 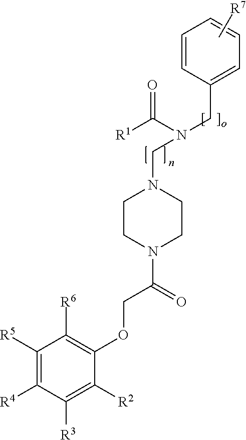 | Ferroptosis inducer by inhibition of cystine uptake by the system xc- cystine-glutamate transporter |
| | | or an enantiomer, optical isomer, | |
| | | diastereomer, N-oxide, crystalline form, | |
| | | hydrate, or pharmaceutically acceptable salt | |
| | | thereof, | |
| | | wherein in the compound of Formula VIII: | |
| | | R₁ is selected from C₁₋₈alkyl, C₁₋₈alkyl-OR³, | |
| | | 3- to 8-membered carbocyclic or | |
| | | heterocyclic, aryl, heteroaryl, C₁₋₄aralkyl, | |
| | | nitrogen substituted with C₁₋₆alkyl, hydroxy | |
| | | substituted C₁₋₆alkyl, and C₁₋₄alkoxy; R₂, R₃, | |
| | | R₄, R₅, and R₆ are independently selected | |
| | | from H, halo, C₁₋₄ alkyl, C₁₋₄ alkylamino, | |
| | | acyl, and alkylsulfonyl; R₇ is selected from | |
| | | halo, C₁₋₈alkyl, C₁₋₈alkylamino, C₁₋₈ | |
| | | alkylthio, C₁₋₈ alkoxy, C₁₋₈ alkynyl, amide, | |
| | | amine, carbamate, carbonate, carboxy, acyl, | |
| | | ether, heteroalkyl, and aralkyl; and n and o | |
| | | are independently selected from an integer | |
| | | from 1 to 4. | |
| | | Formula IX: | |
| erastin- related (Formula VII) | | 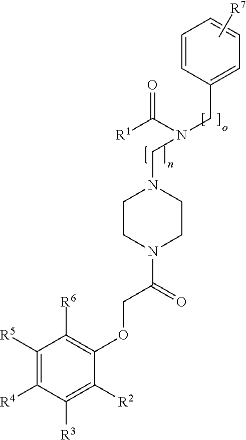 | Ferroptosis inducer by inhibition of cystine uptake by the system xc- cystine-glutamate transporter |
| | | or an enantiomer, optical isomer, | |
| | | diastereomer, N-oxide, crystalline form, | |
| | | hydrate, or pharmaceutically acceptable salt | |
| | | thereof, | |
| | | wherein in the compound of Formula IX, R₁ | |
| | | is selected from methyl, ethyl, propyl, | |
| | | phenyl, and a substituted N; R₂, R₃, R₄, R₅, | |
| | | and R₆ are independently selected from H, | |
| | | halo, C₁₋₄alkyl, C₁₋₄ alkylamino, acyl, and | |
| | | alkylsulfonyR₇ is F; n is 2; o is 1 | |
| | | Formula X: | |
| RSL-like compound | | 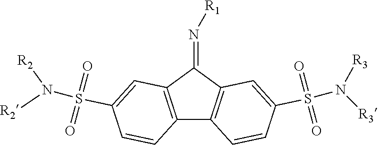 | Ferroptosis inducer by inhibition of cystine uptake by the system xc- cystine-glutamate transporter |
| | | or an N-oxide, crystalline form, hydrate, or | |
| | | pharmaceutically acceptable salt thereof; | |
| | | wherein in the compound of Formula X: R₁ | |
| | | is selected from the group consisting of H, | |
| | | OH, and (—OCH₂CH₂)ₓOH; X is an integer | |
| | | from 1 to 6; and R₂, R_{2′}, R₃, and R_{3′} | |
| | | independently are selected from the group | |
| | | consisting of H, C₃₋₈ cycloalkyl, and | |
| | | combinations thereof, or R₂ and R_{2′} may be | |
| | | joined together to form a pyridinyl or | |
| | | pyranyl and R₃ and R_{3′} may be joined | |
| | | together to form a pyridinyl or pyranyl; or an | |
| | | N-oxide, or pharmaceutically acceptable salt | |
| | | thereof. | |
| | | Formula XI: | |
| RSL-like compound | | 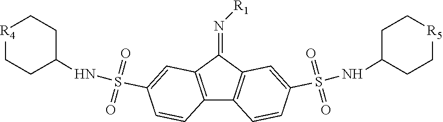 | Ferroptosis inducer by GPX4 inhibition |
| | | wherein in the compound of Formula XI, R₁ | |
| | | is selected from the group consisting of OH | |
| | | and —(OCH₂CH₂)ₓOH; X is an integer from | |
| | | 1 to 6; and R₄ and R₅ are independently | |
| | | selected from the group consisting of CH₂ | |
| | | and O; or an N-oxide; or | |
| | | wherein: R₁ is OH and (1) R₄ and R₅ are both | |
| | | O; or (2) R₄ is CH₂ and R₅ is O; or (3) R₄ is | |
| | | O and R₅ is CH₂ or an N-oxide, or | |
| | | pharmaceutically acceptable salt thereof. | |
| RSL-like compound SRS8-18 | | 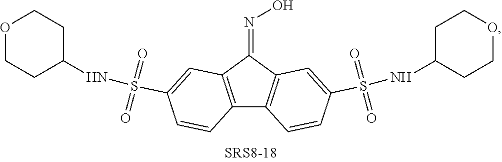 [SMILES: O=S(=O)(NC1CCOCC1)c1ccc2c(c1)C(=NO)c1cc(S(=O)(=O)NC3CCOCC3)ccc1-2] | Ferroptosis inducer by GPX4 inhibition |
| RSL-like compound SRS11-31 | | 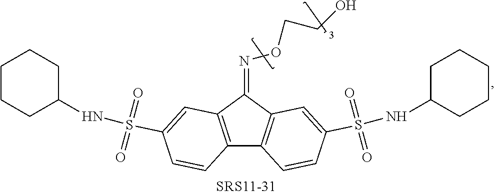 | Ferroptosis inducer by GPX4 inhibition |
| RSL-like compound SRS11-66 | | 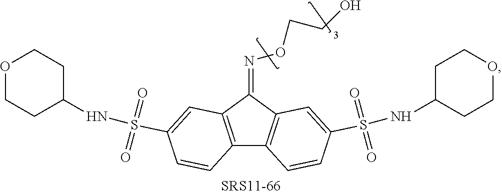 | Ferroptosis inducer by GPX4 inhibition |
| | | Formula XII: | |
| Ferroptosis inducer | | 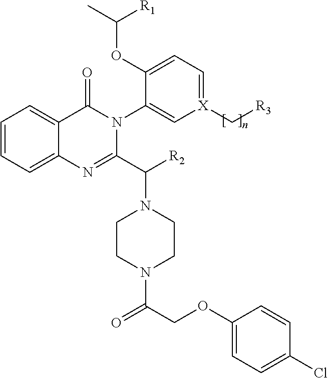 | Ferroptosis inducer by GSH inhibition |
| | | wherein in the compoud of Formula XII, R₁ | |
| | | is selected from the group consisting of H, | |
| | | C₁₋₄ alkyl, C₁₋₄ alkoxy, hydroxy, and halogen; | |
| | | R₂ is selected from the group consisting of | |
| | | H, C₁₋₄ alkyl, C₁₋₄ alkoxy, C₃₋₈ cycloalkyl, C₃₋₈ | |
| | | heterocycloalkyl, aryl, heteroaryl, C₁₋₄ | |
| | | aralkyl; R₃ is selected from the group | |
| | | consisting of nothing, C₁₋₄ alkyl, C₁₋₄ alkoxy, | |
| | | carbonyl, C₃₋₈ cycloalkyl, and C₃₋₈ | |
| | | heterocycloalkyl; X is selected from the | |
| | | group consisting of C, N, and O; and n is an | |
| | | integer from 0-6, with the proviso that when | |
| | | X is C, n = 0, and R₃ is nothing, R₁ cannot be | |
| | | H when R₂ is CH₃, or an N-oxidecrystalline | |
| | | form, hydrate, or pharmaceutically | |
| | | acceptable salt thereof. | |
| | | Formula XIII: | |
| Ferroptosis Inducer | | 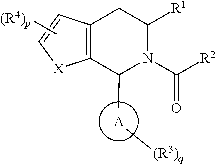 | Ferroptosis inducer by GPX4 inhibition |
| | | wherein in the compoud of Formula XIII: | |
| | | ring A is C₄-C₁₀cycloalkyl, heterocyclyl, aryl, | |
| | | or heteroaryl; X is —O—, —S—, —NR₉—, | |
| | | —CR⁵═CR⁵—, or —CR⁵═N—; p is 0, 1 or | |
| | | 2; q is 0, 1, 2 or 3; R₁ is C₁-C₆alkyl, C₂- | |
| | | C₆alkenyl, C₂-C₆alkynyl, C₁-C₆haloalkyl, C₃- | |
| | | C₁₀cycloalkyl, —CN, —OR⁷, —C(O)OR⁶, | |
| | | —C(O)N(R⁷)₂, —OC(O)R⁶, —S(O)₂R⁸, | |
| | | —S(O)₂N(R⁷)₂, —S(O)N(R⁷)₂, —S(O)R⁸, | |
| | | —N(R⁷)₂, —NO₂, -C₁-C₂alkyl-OR⁷, or | |
| | | —Si(R¹⁵)₃; R₂ is -C₁-C₂haloalkyl, -C₂- | |
| | | C₃alkenyl, -C₂-C₃haloalkenyl, C₂alkynyl, | |
| | | or —CH₂OS(O)₂-phenyl, wherein the C₁- | |
| | | C₂alkylhalo and -C₂-C₃alkenylhalo are | |
| | | optionally substituted with one or two —CH₃, | |
| | | and the C₂alkynyl and phenyl are optionally | |
| | | substituted with one —CH₃; each R₃ is | |
| | | independently halo, —CN, —OH, —OR⁸, | |
| | | —NH₂, —NHR⁸, —N(R⁸)₂, —S(O)₂R⁸, | |
| | | —S(O)R⁸, —S(O)₂N(R⁷)₂, —S(O)N(R⁷)₂, | |
| | | —NO₂, —Si(R¹²)₃, —SF₅, —C(O)OR⁶, | |
| | | —C(O)N(R⁷)₂, —NR¹²C(O)R₈, | |
| | | —NR¹²C(O)OR⁸, —OC(O)N(R⁷)₂, | |
| | | —OC(O)R⁸, —C(O)R⁶, | |
| | | —OC(O)CHR⁸N(R¹²)₂, C₁-C₆alkyl, C₂- | |
| | | C₆alkenyl, C₂-C₆alkynyl, C₃-C₁₀cycloalkyl, | |
| | | heterocyclyl, aryl, heteroaryl, -C₁- | |
| | | C₆alkylC₃-C₁₀cycloalkyl, -C₂-C₆alkenylC₃- | |
| | | C₁₀cycloalkyl, -C₁-C₆alkylheterocyclyl, | |
| | | -C₂-C₆alkenylheterocyclyl, -C₁-C₆alkylaryl, | |
| | | -C₂-C₆alkenylaryl, C₁-C₆alkylheteroaryl, | |
| | | or -C₂-C₆alkenylheteroaryl; wherein each | |
| | | C₁-C₆alkyl, C₂-C₆alkenyl, C₂-C₆alkynyl, C₃- | |
| | | C₁₀cycloalkyl, heterocyclyl, aryl, heteroaryl, | |
| | | -C₁-C₆alkyl C₃-C₁₀cycloalkyl, -C₂- | |
| | | C₆alkenylC₃-C₁₀cycloalkyl, -C₁- | |
| | | C₆alkylheterocyclyl, -C₂- | |
| | | C₆alkenylheterocyclyl, -C₁-C₆alkylaryl, | |
| | | -C₂-C₆alkenylaryl, -C₁-C₆alkylheteroaryl, | |
| | | or -C₂-C₆alkenylheteroaryl of R₃ is | |
| | | independently optionally substituted with | |
| | | one to three R₁₀; each R₄ is independently | |
| | | halo, —CN, —OH, —OR⁸, —NH₂, | |
| | | —NHR⁸, —N(R⁸)₂, —S(O)₂R⁸, —S(O)R⁸, | |
| | | —S(O)₂N(R)₂, —S(O)N(R)₂, —NO₂, | |
| | | —Si(R⁵)₃, —C(O)OR⁶, —C(O)N(R⁷)₂, | |
| | | —NR¹²C(O)R⁸, —OC(O)R, —C(O)R⁶, | |
| | | —NR¹²C(O)OR⁸, —OC(O)N(R⁷)₂, | |
| | | —OC(O)CHR⁸N(R¹²)₂, C₁-C₆alkyl, C₂- | |
| | | C₆alkenyl, C₂-C₆alkynyl, C₃-C₁₀cycloalkyl, | |
| | | heterocyclyl, aryl, heteroaryl, -C₁- | |
| | | C₆alkylC₃-C₁₀cycloalkyl, -C₂-C₆alkenylC₃- | |
| | | C₁₀cycloalkyl, -C₁-C₆alkylheterocyclyl, | |
| | | -C₂-C₆alkenylheterocyclyl, -C₁-C₆alkylaryl, | |
| | | C₂-C₆alkenylaryl, C₁-C₆alkylheteroaryl, | |
| | | or-C₂-C₆alkenylheteroaryl; wherein each | |
| | | C₁-C₆alkyl, C₂-C₆alkenyl, C₂-C₆alkynyl, C₃- | |
| | | C₁₀cycloalkyl, heterocyclyl, aryl, heteroaryl, | |
| | | -C₁-C₆alkylC₃-C₁₀cycloalkyl, -C₂- | |
| | | CoalkenylC₃-C₁₀cycloalkyl, -C₁- | |
| | | C₆alkylheterocyclyl, -C₂- | |
| | | C₆alkenylheterocyclyl, -C₁-C₆alkylaryl, | |
| | | -C₂-C₆alkenylaryl, C₁-C₆alkylheteroaryl, of | |
| | | -C₂-C₆alkenylheteroaryl of R⁴ is optionally | |
| | | independently optionally substituted with | |
| | | one to three R¹⁰; | |
| | | each R⁵ is independently hydrogen, halo, | |
| | | —CN, —OH, —OR⁸, —NH₂, —NHR⁸, | |
| | | —N(R⁸)₂, —S(O)₂R⁸, —S(O)R⁸, | |
| | | —S(O)₂N(R⁷)₂, —S(O)N(R⁷)₂, —NO₂, | |
| | | —Si(R⁵)₃, —C(O)OR⁶, —C(O)N(R⁷)₂, | |
| | | —NR¹²C(O)R⁸, —OC(O)R⁸, —C(O)R⁶, | |
| | | —NR¹²C(O)OR⁸, —OC(O)N(R⁷)₂, | |
| | | —OC(O)CHR⁸N(R¹²)₂, C₁-C₆alkyl, C₂- | |
| | | C₆alkenyl, C₂-C₆alkynyl, C₃-C₁₀cycloalkyl, | |
| | | heterocyclyl, aryl, heteroaryl, -C₁- | |
| | | C₆alkylC₃-C₁₀cycloalkyl, -C₂-C₆alkenylC₃- | |
| | | C₁₀cycloalkyl, -C₁-C₆alkylheterocyclyl, | |
| | | -C₂-C₆alkenylheterocyclyl, -C₁-C₆alkylaryl, | |
| | | -C₂-C₆alkenylaryl, C₁-C₆alkylheteroaryl, | |
| | | or -C₂-C₆alkenylheteroaryl; wherein each | |
| | | C₁-C₆alkyl, C₂-C₆alkenyl, C₂-C₆alkynyl, C₃- | |
| | | C₁₀cycloalkyl, heterocyclyl, aryl, heteroaryl, | |
| | | -C₁-C₆alkylC₃-C₁₀cycloalkyl, -C₂- | |
| | | C₆alkenylC₃-C₁₀cycloalkyl, -C₁- | |
| | | C₆alkylheterocyclyl, -C₂- | |
| | | C₆alkenylheterocyclyl, -C₁-C₆alkylaryl, | |
| | | -C₆-C₆alkenylaryl, C₁-C₆alkylheteroaryl, or | |
| | | -C₂-C₆alkenylheteroaryl of R is optionally | |
| | | independently optionally substituted with | |
| | | one to three R¹⁰; | |
| | | each R⁶ is independently hydrogen, C₁- | |
| | | C₆alkyl, C₂-C₆alkenyl, C₂-C₆alkynyl, C₃- | |
| | | C₁₀cycloalkyl, heterocyclyl, aryl, heteroaryl, | |
| | | -C₁-C₆alkylC₃-C₁₀cycloalkyl, -C₂- | |
| | | C₆alkenylC₃-C₁₀cycloalkyl, -C₁- | |
| | | C₆alkylheterocyclyl, -C₂- | |
| | | C₆alkenylheterocyclyl, -C₁-C₆alkylaryl, | |
| | | -C₂-C₆alkenylaryl, C₁-C₆alkylheteroaryl, or | |
| | | -C₂-C₆alkenylheteroaryl; wherein each R⁶ is | |
| | | independently further substituted with one to | |
| | | three R¹¹; | |
| | | each R⁷ is independently hydrogen, C₁- | |
| | | C₆alkyl, C₂-C₆alkenyl, C₂-C₆alkynyl, C₃- | |
| | | C₁₀cycloalkyl, heterocyclyl, aryl, heteroaryl, | |
| | | -C₁-C₆alkylC₃-C₆cycloalkyl, -C₂- | |
| | | C₆alkenylC₃-C₆cycloalkyl, -C₁- | |
| | | C₆alkylheterocyclyl, -C₂- | |
| | | C₆alkenylheterocyclyl, -C₁-C₆alkylaryl, | |
| | | -C₂-C₆alkenylaryl, -C₁-C₆alkylheteroaryl, | |
| | | -C₂-C₆alkenylheteroaryl, or two R⁷ together | |
| | | with the nitrogen atom to which they are | |
| | | attached, form a 4 to 7 membered | |
| | | heterocyclyl; wherein each R⁷ or ring formed | |
| | | thereby is independently further substituted | |
| | | with one to three R¹¹; | |
| | | each R⁸ is independently C₁-C₆alkyl, C₂- | |
| | | C₆alkenyl, C₂-C₆alkynyl, C₃-C₁₀cycloalkyl, | |
| | | heterocyclyl, aryl, heteroaryl, -C₁- | |
| | | C₆alkylC₃-C₁₀cycloalkyl, -C₂-C₆alkenylC₃- | |
| | | C₁₀cycloalkyl, -C₁-C₆alkylheterocyclyl, | |
| | | -C₂-C₆alkenylheterocyclyl, -C₂-C₆alkylaryl, | |
| | | -C₂-C₆alkenylaryl, -C₁-C₆alkylheteroaryl, | |
| | | or -C₂-C₆alkenylheteroaryl; wherein each | |
| | | R⁸ is independently further substituted with | |
| | | one to three R¹¹; | |
| | | R⁹ is hydrogen or C₁-C₆alkyl; | |
| | | each R¹⁰ is independently halo, —CN, | |
| | | —OR¹², —NO₂,— N(R¹²)₂, —S(O)R³, | |
| | | —S(O)2R¹³, —S(O)N(R¹²)₂, —S(O)₂N(R¹²)₂, | |
| | | —Si(R¹²)3, —C(O)R¹², —C(O)OR¹², | |
| | | —C(O)N(R¹²)₂, —NR¹²C(O)R¹², —OC(O)R¹², | |
| | | —OC(O)OR¹², —OC(O)N(R¹²)₂, | |
| | | —NR¹²C(O)OR¹², —OC(O)CHR¹²N(R¹²)₂, C₁- | |
| | | C₆alkyl, C₁-C₆haloalkyl, C₂-C₆alkenyl, C₂- | |
| | | C₆alkynyl, C₃-C₁₀cycloalkyl, heterocyclyl, | |
| | | aryl, or heteroaryl, wherein each C₁-C₆alkyl, | |
| | | C₁-C₆haloalkyl, C₂-C₆alkenyl, C₂-C₆alkynyl, | |
| | | C₃-C₁₀cycloalkyl, heterocyclyl, aryl, or | |
| | | heteroaryl of R¹⁰ is optionally independently | |
| | | substituted with one to three R¹¹; | |
| | | each R¹¹ is independently halo, —CN, | |
| | | —OR¹², —NO₂, —N(R¹²)₂, —S(O)R³, | |
| | | —S(O)₂R¹³, —S(O)N(R¹²)₂, —S(O)₂N(R¹²)₂, | |
| | | —Si(R¹²)₃, —C(O)R¹², —C(O)OR¹², | |
| | | —C(O)N(R¹²)₂, —NR¹²C(O)R¹², —OC(O)R¹², | |
| | | —OC(O)OR¹², —OC(O)N(R¹²)₂, | |
| | | —NR¹²C(O)OR¹², —OC(O)CHR¹²N(R¹²)₂, C₁- | |
| | | C₆alkyl, C₁-C₆haloalkyl, C₂-C₆alkenyl, C₂- | |
| | | C₆alkynyl, C₃-C₁₀cycloalkyl, heterocyclyl, | |
| | | aryl, or heteroaryl; | |
| | | each R¹² is independently hydrogen, C₁- | |
| | | C₆alkyl or C₃-C₁₀cycloalkyl; | |
| | | each R¹³ is independently C₁-C₆alkyl or C₃- | |
| | | C₁₀cycloalkyl; and | |
| | | each R¹⁵ is independently C₁-C₆alkyl, C₂- | |
| | | C₆alkenyl, aryl, heteroaryl, -C₁-C₆alkylaryl, | |
| | | -C₂-C₆alkenylaryl, -C₁-C₆alkylheteroaryl, | |
| | | and -C₂-C₆alkenylheteroaryl; provided that | |
| | | at least one of the following is true: | |
| | | 1) R¹ is other than —C(O)OCH₃; | |
| | | 2) R² is -C₂alkynyl optionally substituted | |
| | | with one —CH₃; or | |
| | | 3) when R¹ is —C(O)OCH₃ and R² is | |
| | | —CH₂Cl, then the moiety | |
| | | 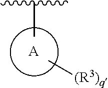 | |
| | | is other than 1,3-benzodioxol-5-yl, 4- | |
| | | nitrophenyl, 4-bromophenyl, cyclohexyl, | |
| | | furyl, or 4-methoxyphenyl. | |
| | | Formula XIV: | |
| Ferroptosis Inducer | | 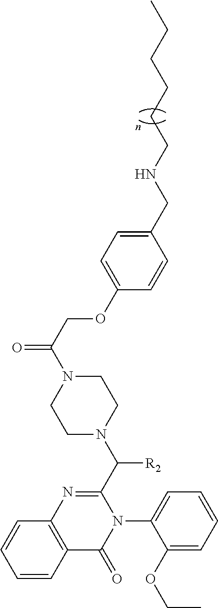 | Induces ferroptosis by binding the Sigma-2 receptor and having GPX4 inhibitor activity. |
| | | or an enantiomer, | |
| | | diastereomer, N-oxide, | |
| | | hydrate, or pharmaceutica. | |
| | | thereof, | |
| | | wherein n is an integer ch | |
| | | 4, 5, and R₂ is H, or CH₃. | |
| | | Formula XV: | |
| Ferroptosis Inducer | | 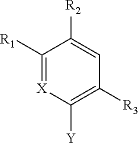 | Ferroptosis inducer by reducing reactive oxygen species (ROS) in a cell |
| | | or an enantiomer, | |
| | | optical isomer, diastereomer, N-oxide, | |
| | | crystalline form, hydrate, or | |
| | | pharmaceutically acceptable salt thereof, | |
| | | wherein X is N; Y is H, halo, or C₁₋₄ alkyl; R₁ | |
| | | is N(R₄R₅), and at least one of R₄ and R₅ has | |
| | | a ring structure as defined below; R₂ is | |
| | | N(R₆R₇); R₃ is selected from the group | |
| | | consisting of H, | |
| | | 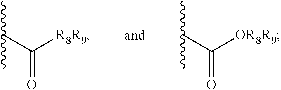 | |
| | | R₄ and R₅ are independently selected from | |
| | | the group consisting of H, C₁₋₁₂alkyl, C₃₋₁₂ | |
| | | cycloalkyl, and aryl, wherein one or more of | |
| | | the ring carbons of the cycloalkyl are | |
| | | optionally substituted with one or more | |
| | | heteroatoms, and the cycloalkyl optionally | |
| | | comprises one or more pendant groups | |
| | | selected from the group consisting of H, F, | |
| | | N(R₁₀R₁₁), Boc, COOR₁₂, and C₁₋₈alkyl; | |
| | | R₆ and R₇ are independently selected from | |
| | | the group consisting of H, C₁₋₆alkyl, Boc, O, | |
| | | COOR₁₂, | |
| | | 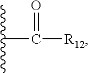 | |
| | | and C₁₋₃ alkyl-aryl, | |
| | | wherein one or more of the ring carbprons of | |
| | | the alkyl-aryl are optionally substituted with | |
| | | one or more nitrogen atoms, and the alkyl- | |
| | | aryl optionally comprises one or more | |
| | | pendant groups selected from the group | |
| | | consisting of H, halo, CN, NO₂, C₁₋₄ ether, | |
| | | C₁₋₄ ester, OCOOR₁₂, and C₁₋₈ alkyl, which | |
| | | C₁₋₈ alkyl is optionally further substituted | |
| | | with one or more halo; R₈ and R₉ are | |
| | | independently selected from the group | |
| | | consisting of no atom, O, N, NHR₁₂, C₁₋₁₀ | |
| | | alkyl, and C₁₋₁₀ ether, wherein the alkyl and | |
| | | the ether are optionally substituted with NH₂, | |
| | | NHBoc, or C₃₋₁₂ cycloalkyl, wherein one or | |
| | | more of the ring carbons of the cycloalkyl | |
| | | are optionally substituted with one or more | |
| | | heteroatoms; R₁₀ and R₁₁ are independently | |
| | | selected from H and Boc; and R₁₂ is a C₁₋₄ | |
| | | alkyl optionally substituted with aryl. | |
| Ferroptosis Modulator X | | 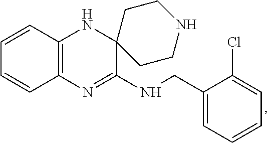 [SMILES: Clc1ccccc1CNC1=Nc2ccccc2NC12CCNCC2] | Modulates ferroptosis by altering reactive oxygen species (ROS) |
| Ferroptosis Modulator Y | | 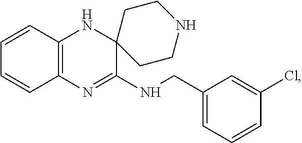 [SMILES: Clc1cccc(CNC2=Nc3ccccc3NC23CCNCC3)c1] | Modulates ferroptosis by altering reactive oxygen species (ROS) |
| FIN56 | C₂₅H₃₁N₃O₅S₂ | | Induces |
| | Cas: | | ferroptosis by: (1) |
| | 1083162- | | promoting the |
| | 61-1 | | degradation of |
| | | | GPX4 and (2) |
| | | | reducing the |
| | | | abundance of |
| | | | CoQ10 (i.e., an |
| | | | antioxidant in the |
| | | | cell) |
| FINO₂ | C₁₅H₂₈O₃ Cas: 869298-31-7 | 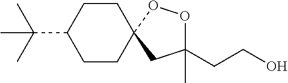 | Induces ferroptosis by increasing lipid peroxidation and inhibiting GPX4 activity |
| glutamate | C₅H₉NO₄ Cas:139883- 82-2 | 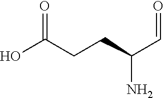 | High extracellular glutamate concentrations prevent cystine import, causes GSH depletion increasing |
| | | | ferroptosis |
| | | | sensitivity and |
| | | | induction |
| GPX4-IN-3 | C₂₉H₂₄ClN₃O₃S | 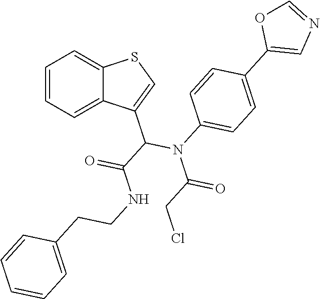 [SMILES: O=C(NCCc1ccccc1)C(c1csc2ccccc12)N(C(=O)CCl)c1ccc(-c2cnco2)cc1] | Ferroptosis inducer by GPX4 inhibition |
| jacaric acid | C₁₈H₃₀O₂ Cas: 28872-28-8 | 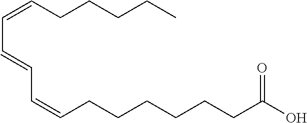 [SMILES: CCCCC\C=C/C=C/C=C\CCCCCCC(=O)O] | Induces ferroptosis by modulating the production of reactive oxygen species |
| JKE-1674 | C₂₀H₂₀Cl₂N₄O₄ Cas: 2421119- 60-8 | 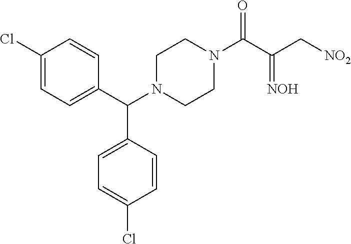 | Ferroptosis inducer by GPX4 inhibition |
| JKE-1716 | C₂₀H₂₀Cl₂N₄O₄ Cas: 2421118- 05-8 | 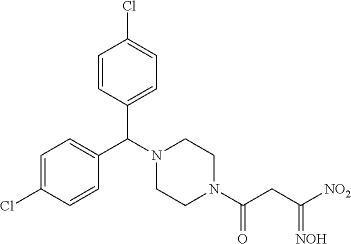 | Ferroptosis inducer by GPX4 inhibition |
| L-buthionine sulfoximine (L-BSO) | C₈H₁₈N₂O₃S Cas: 83730-53-4 | 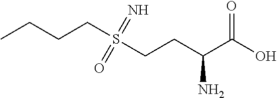 | Ferroptosis inducer by GCL and GSS inhibition |
| ML-162 | C₂₃H₂₂Cl₂N₂O₃S Cas: 1035072- 16-2 | 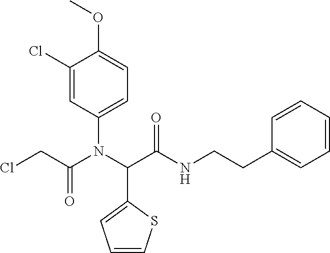 [SMILES: COc1ccc(N(C(=O)CCl)C(C(=O)NCCc2ccccc2)c2cccs2)cc1Cl] | Ferroptosis inducer by GPX4 inhibition |
| ML-210 | C₂₂H₂₀Cl₂N₄O₄ Cas: 1360705- 96-9 | 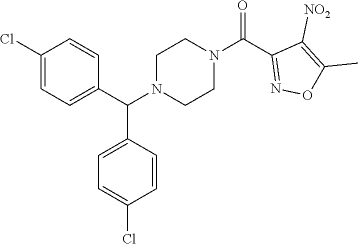 [SMILES: Cc1onc(C(=O)N2CCN(C(c3ccc(Cl)cc3)c3ccc(Cl)cc3)CC2)c1[N+](=O)[O-]] | Ferroptosis inducer by GPX4 inhibition |
| | | Formula XVI: | |
| RSL3-like | | 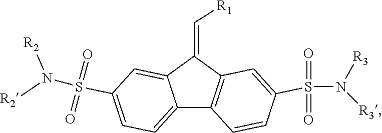 | Ferroptosis inducer by GPX4 inhibition |
| | | wherein in the compound of Formula XVI, | |
| | | R₁ is selected from the group consisting of | |
| | | H, OH, and —(OCH₂CH₂)ₓOH; X is an | |
| | | integer from 1 to 6; and R₂, R_{2′}, R₃, and R_{3′} | |
| | | independently are selected from the group | |
| | | consisting of H, C₃₋₈ cycloalkyl, and | |
| | | combinations thereof, or R₂ and R_{2′} may be | |
| | | joined together to form a pyridinyl or | |
| | | pyranyl and R₃ and R_{3′} may be joined | |
| | | together to form a pyridinyl or pyranyl. | |
| | | Formula XVII: | |
| RSL3-like | | 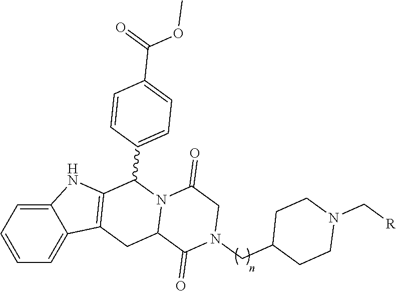 | Ferroptosis inducer by GPX4 inhibition |
| | | or an N-oxide, crystalline form, hydrate, or | |
| | | pharmaceutically acceptable salt thereof; | |
| | | wherein in the compound of Formula XVII: | |
| | | n is 2, 3 or 4; and R is a substituted or | |
| | | unsubstituted C_{1-C6} alkyl group, a substituted | |
| | | or unsubstituted C₃-C₁₀ cycloalkyl group, a | |
| | | substituted or unsubstituted C₂-C₈ | |
| | | heterocycloalkyl group, a substituted or | |
| | | unsubstituted C₆-C₁₀ aromatic ring group, or | |
| | | a substituted or unsubstituted C₃-C₈ | |
| | | heteroaryl ring group; wherein the | |
| | | substitution means that one or more | |
| | | hydrogen atoms in each group are substituted | |
| | | by the following groups selected from the | |
| | | group consisting of: halogen, cyano, nitro, | |
| | | hydroxy, C₁-C₆ alkyl, halogenated C₁-C₆ | |
| | | alkyl, C₁-C₆ alkoxy, halogenated C₁-C₆ | |
| | | alkoxy, COOH (carboxy), COOC₁-C₆ alkyl, | |
| | | OCOC₁-C₆ alkyl. | |
| Se- Methylseleno cysteine (hydrochloride) | C₄H₉NO₂Se .HCl Cas: 863394-07-4 | 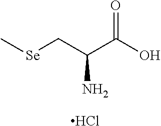 | Ferroptosis inducer by preventing increases in glutathione reductase and glutathione peroxidase (GPX) activity |
| simvastatin | C₂₅H₃₈O₅ Cas: 79902-63-9 | 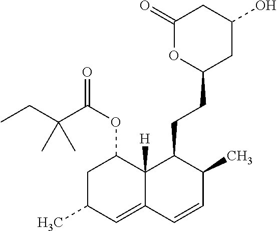 [SMILES: CCC(C)(C)C(=O)O[C@H]1C[C@@H](C)C=C2C=C[C@H](C)[C@H](CC[C@@H]3C[C@@H](O)CC(=O)O3)[C@H]21] | Ferroptosis inducer by GPX4 inhibition |
| sorafenib | C₂₁H₁₆ClF₃N₄O₃ Cas: 284461-73-0 | 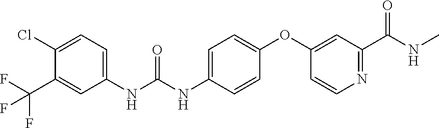 [SMILES: CNC(=O)c1cc(Oc2ccc(NC(=O)Nc3ccc(Cl)c(C(F)(F)F)c3)cc2)ccn1] | Induces ferroptosis by inhibiting multiple kinases |
| sulfasalazine | C₁₈H₁₄N₄O₅S Cas: 599- 79-1 | 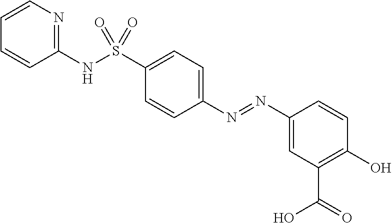 [SMILES: O=C(O)c1cc(/N=N/c2ccc(S(=O)(=O)Nc3ccccn3)cc2)ccc1O] | Ferroptosis inducer by xCT inhibition |
| trigonelline | C₇H₈NO₂ .Cl Cas: 6138- 41-6 | 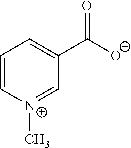 | Ferroptosis inducer by NRF2 inhibition. NRF2 is a transcriptional regulator of GPX4 protein content |

In some embodiments, the ferroptosis-inducing agent which can be a second active agent or a second therapeutic agent can be: (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO₂, CIL56, dihydroisotanshinone I, GPX4-IN-3, analogs, salts, or derivatives thereof. In some embodiments, the agent in Table 1 is a pharmaceutically acceptable salt form of the small molecule.

In some instances, the therapeutic agent, contacting agent, inhibiting agent, partially inhibiting agent, or modulating agent, or compound can be a compound that is one or more of the following compounds in Table 2 or Table 3, an enantiomer or diastereomer of any of the foregoing, a mixture of more than one enantiomers or diastereomers, a pharmaceutically acceptable salt of any of the foregoing, or a deuterated derivative of any of these.

In some embodiments, biological activity of a compound described herein may be measured as an IC₅₀ value. The IC₅₀ value may be between 0.0001 to 0.01, 0.01 to 0.1, 0.1 to 1, or 1 to 10 mM. In some embodiments, the IC₅₀ value may be at least 0.0001, at least 0.001, at least 0.01, at least 0.1, at least 1.0, or at least 10 mM. In some embodiments, biological activity of a compound described herein may be measured as an EC₅₀ value or EC50 value. The EC₅₀ value may be between 0.0001 to 0.01, 0.01 to 0.1, 0.1 to 1, 1 to 10 mM. In some embodiments, the EC₅₀ value may be at least 0.0001, at least 0.001, at least 0.01, at least 0.1, at least 1.0, or at least 10 mM. In Table 2, the following meanings include:
+++ is a GCL IC₅₀ range of less than or equal to 0.02 mM
++ is a GCL IC₅₀ range of greater than 0.02 mM to 2 mM
+ is a GCL IC₅₀ range of greater than 2 mM
“nd” is no data collected
@ is cell killing EC₅₀ greater than or equal to 1 mM
! is cell killing EC₅₀ less than 1 mM
“nd” indicates no data collected

Testing protocols for the biological activity data are described in Example 2.

TABLE 2
Biological Activity Data

| | | GCL IC₅₀ | Cell Killing |
|---|---|---|---|
| Compound # | | (mM) | EC₅₀ (mM) |
| 1 | 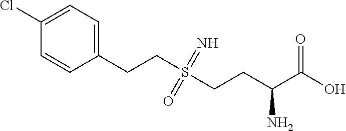 [SMILES: N=S(=O)(CCc1ccc(Cl)cc1)CC[C@H](N)C(=O)O] | +++ | @ |
| 2 | 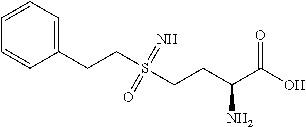 [SMILES: N=S(=O)(CCc1ccccc1)CC[C@H](N)C(=O)O] | ++ | @ |
| 3 | 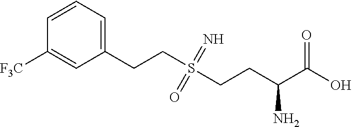 [SMILES: N=S(=O)(CCc1cccc(C(F)(F)F)c1)CC[C@H](N)C(=O)O] | ++ | @ |
| 4 | 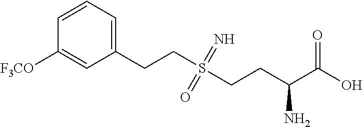 [SMILES: N=S(=O)(CCc1cccc(OC(F)(F)F)c1)CC[C@H](N)C(=O)O] | ++ | @ |
| 5 | 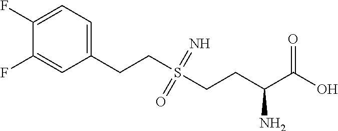 [SMILES: N=S(=O)(CCc1ccc(F)c(F)c1)CC[C@H](N)C(=O)O] | +++ | @ |
| 6 | 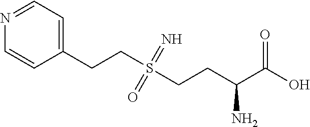 [SMILES: N=S(=O)(CCc1ccncc1)CC[C@H](N)C(=O)O] | +, ++ (2.27, >1.62) | @ |
| 7 | 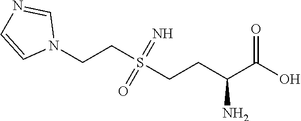 [SMILES: N=S(=O)(CC[C@H](N)C(=O)O)CCn1ccnc1] | + | nd |
| 8 | 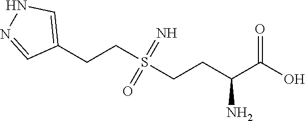 [SMILES: N=S(=O)(CCc1cn[nH]c1)CC[C@H](N)C(=O)O] | ++ | @ |
| 9 | 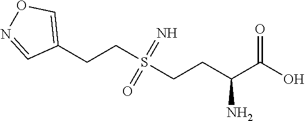 [SMILES: N=S(=O)(CCc1cnoc1)CC[C@H](N)C(=O)O] | ++ | ! |
| 10 | 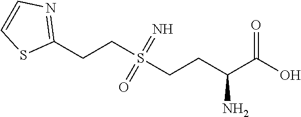 [SMILES: N=S(=O)(CCc1nccs1)CC[C@H](N)C(=O)O] | + | @ |
| 11 | 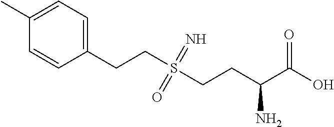 [SMILES: Cc1ccc(CCS(=N)(=O)CC[C@H](N)C(=O)O)cc1] | +++ | @ |
| 12 | 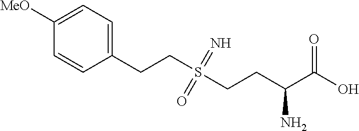 [SMILES: COc1ccc(CCS(=N)(=O)CC[C@H](N)C(=O)O)cc1] | ++ | @ |
| 13 | 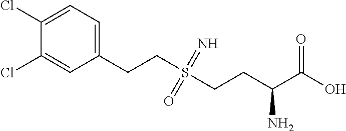 [SMILES: N=S(=O)(CCc1ccc(Cl)c(Cl)c1)CC[C@H](N)C(=O)O] | +++ | @ |
| 14 | 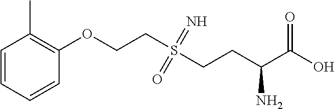 [SMILES: Cc1ccccc1OCCS(=N)(=O)CC[C@H](N)C(=O)O] | ++ | nd |
| 15 | 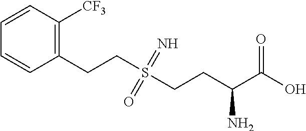 [SMILES: N=S(=O)(CCc1ccccc1C(F)(F)F)CC[C@H](N)C(=O)O] | +++ | @ (1.2, 0.97) |
| 16 | 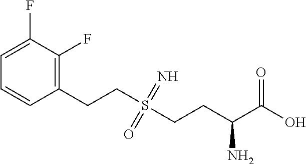 [SMILES: N=S(=O)(CCc1cccc(F)c1F)CC[C@H](N)C(=O)O] | +++ | ! |
| 17 | 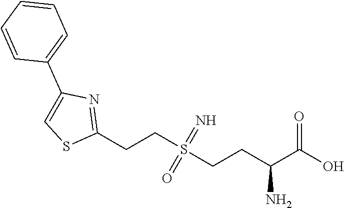 [SMILES: N=S(=O)(CCc1nc(-c2ccccc2)cs1)CC[C@H](N)C(=O)O] | ++ | @ |
| 18 | 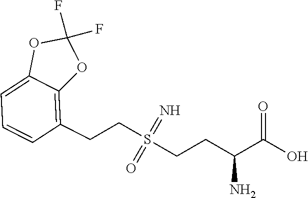 [SMILES: N=S(=O)(CCc1cccc2c1OC(F)(F)O2)CC[C@H](N)C(=O)O] | ++ | @ |
| 19 | 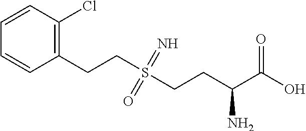 [SMILES: N=S(=O)(CCc1ccccc1Cl)CC[C@H](N)C(=O)O] | +++ | ! |
| 20 | 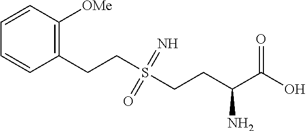 [SMILES: COc1ccccc1CCS(=N)(=O)CC[C@H](N)C(=O)O] | ++ | @ |
| 21 | 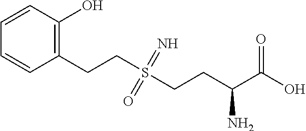 [SMILES: N=S(=O)(CCc1ccccc1O)CC[C@H](N)C(=O)O] | +++ | ! |
| 22 | 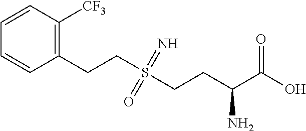 [SMILES: N=S(=O)(CCc1ccccc1C(F)(F)F)CC[C@H](N)C(=O)O] | +++ | @ |
| 23 | 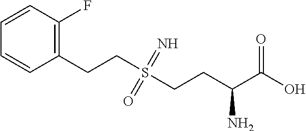 [SMILES: N=S(=O)(CCc1ccccc1F)CC[C@H](N)C(=O)O] | ++ | @ |
| 24 | 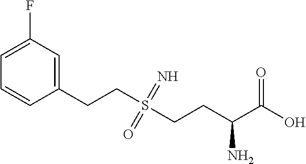 [SMILES: N=S(=O)(CCc1cccc(F)c1)CC[C@H](N)C(=O)O] | +++ | @ |
| 25 | 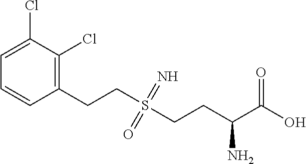 [SMILES: N=S(=O)(CCc1cccc(Cl)c1Cl)CC[C@H](N)C(=O)O] | ++ | @ |
| 26 | 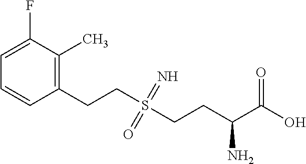 [SMILES: Cc1c(F)cccc1CCS(=N)(=O)CC[C@H](N)C(=O)O] | +++ | ! |
| 27 | 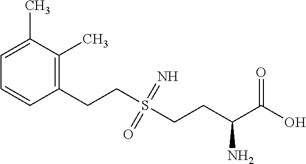 [SMILES: Cc1cccc(CCS(=N)(=O)CC[C@H](N)C(=O)O)c1C] | ++ | @ |
| 28 | 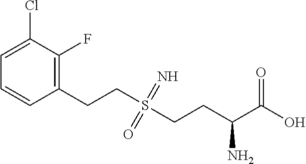 [SMILES: N=S(=O)(CCc1cccc(Cl)c1F)CC[C@H](N)C(=O)O] | +++ | @ |
| 29 | 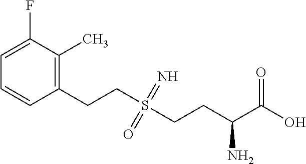 [SMILES: Cc1c(F)cccc1CCS(=N)(=O)CC[C@H](N)C(=O)O] | +++ | @ |
| 30 | 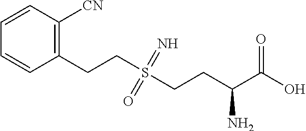 [SMILES: N#Cc1ccccc1CCS(=N)(=O)CC[C@H](N)C(=O)O] | +++ | @ |
| 31 | 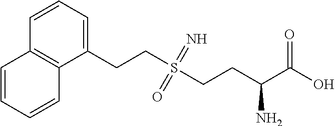 [SMILES: N=S(=O)(CCc1cccc2ccccc12)CC[C@H](N)C(=O)O] | +++ | @ |
| 32 | 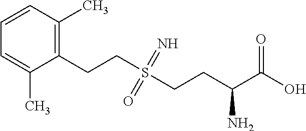 [SMILES: Cc1cccc(C)c1CCS(=N)(=O)CC[C@H](N)C(=O)O] | ++ | @ |
| 33 | 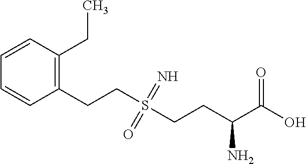 [SMILES: CCc1ccccc1CCS(=N)(=O)CC[C@H](N)C(=O)O] | +++ | @ |
| 34 | 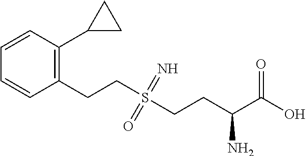 [SMILES: N=S(=O)(CCc1ccccc1C1CC1)CC[C@H](N)C(=O)O] | ++ | @ |
| 35 | 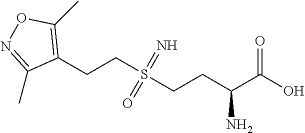 [SMILES: Cc1noc(C)c1CCS(=N)(=O)CC[C@H](N)C(=O)O] | ++ | @ |
| 36 | 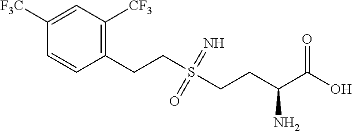 [SMILES: N=S(=O)(CCc1ccc(C(F)(F)F)cc1C(F)(F)F)CC[C@H](N)C(=O)O] | +++ | ! |
| 37 | 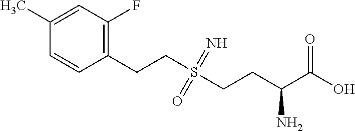 [SMILES: Cc1ccc(CCS(=N)(=O)CC[C@H](N)C(=O)O)c(F)c1] | +++ | @ |
| 38 | 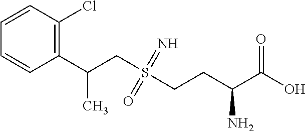 [SMILES: CC(CS(=N)(=O)CC[C@H](N)C(=O)O)c1ccccc1Cl] | ++ | @ |
| 39 | 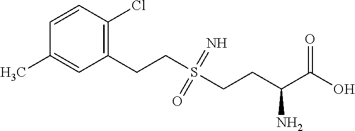 [SMILES: Cc1ccc(Cl)c(CCS(=N)(=O)CC[C@H](N)C(=O)O)c1] | +++ | @ |
| 40 | 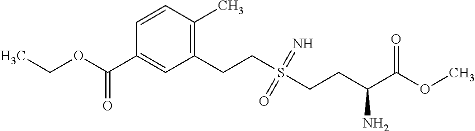 [SMILES: CCOC(=O)c1ccc(C)c(CCS(=N)(=O)CC[C@H](N)C(=O)OC)c1] | ++ | @ |
| 41 | 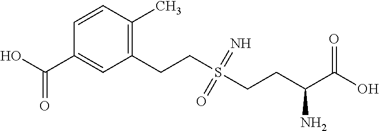 [SMILES: Cc1ccc(C(=O)O)cc1CCS(=N)(=O)CC[C@H](N)C(=O)O] | ++ | @ |
| 42 | 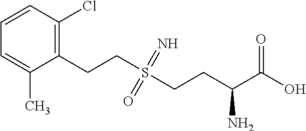 [SMILES: Cc1cccc(Cl)c1CCS(=N)(=O)CC[C@H](N)C(=O)O] | ++ | @ |
| 43 | 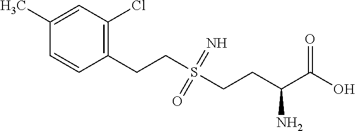 [SMILES: Cc1ccc(CCS(=N)(=O)CC[C@H](N)C(=O)O)c(Cl)c1] | +++ | @ |
| 44 | 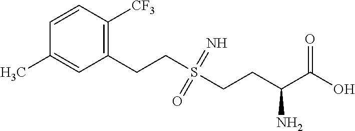 [SMILES: Cc1ccc(C(F)(F)F)c(CCS(=N)(=O)CC[C@H](N)C(=O)O)c1] | +++ | @ |
| 45 | 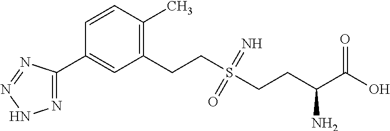 [SMILES: Cc1ccc(-c2nn[nH]n2)cc1CCS(=N)(=O)CC[C@H](N)C(=O)O] | +++ | @ |
| 46 | 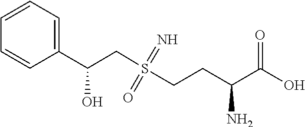 [SMILES: N=S(=O)(CC[C@H](N)C(=O)O)C[C@H](O)c1ccccc1] | ++ | @ |
| 47 | 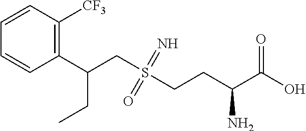 [SMILES: CCC(CS(=N)(=O)CC[C@H](N)C(=O)O)c1ccccc1C(F)(F)F] | + | @ |
| 48 | 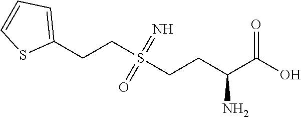 [SMILES: N=S(=O)(CCc1cccs1)CC[C@H](N)C(=O)O] | +++ | @ |
| 49 | 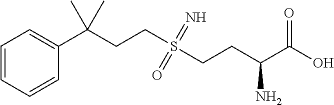 [SMILES: CC(C)(CCS(=N)(=O)CC[C@H](N)C(=O)O)c1ccccc1] | ++ | @ |
| 50 | 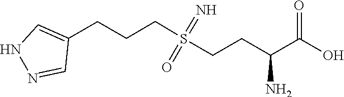 [SMILES: N=S(=O)(CCCc1cn[nH]c1)CC[C@H](N)C(=O)O] | ++ | nd |
| 51 | 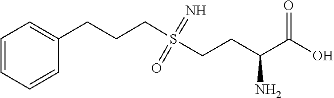 [SMILES: N=S(=O)(CCCc1ccccc1)CC[C@H](N)C(=O)O] | ++ | @ |
| 52 | 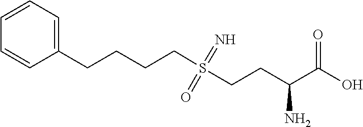 [SMILES: N=S(=O)(CCCCc1ccccc1)CC[C@H](N)C(=O)O] | ++ | @ |
| 53 | 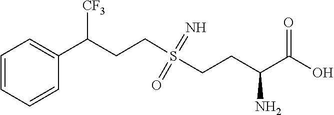 [SMILES: N=S(=O)(CCC(c1ccccc1)C(F)(F)F)CC[C@H](N)C(=O)O] | ++ | ! |
| 54 | 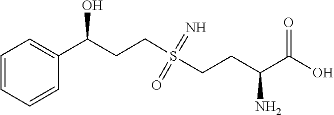 [SMILES: N=S(=O)(CC[C@H](N)C(=O)O)CC[C@H](O)c1ccccc1] | ++ | @ |
| 55 | 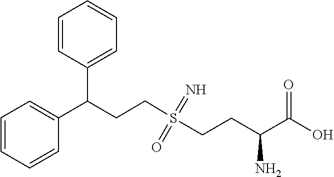 [SMILES: N=S(=O)(CCC(c1ccccc1)c1ccccc1)CC[C@H](N)C(=O)O] | ++ | @ |
| 56 | 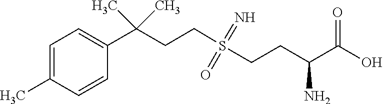 [SMILES: Cc1ccc(C(C)(C)CCS(=N)(=O)CC[C@H](N)C(=O)O)cc1] | ++ | @ |
| 57 | 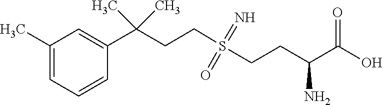 [SMILES: Cc1cccc(C(C)(C)CCS(=N)(=O)CC[C@H](N)C(=O)O)c1] | ++ | @ |
| 58 | 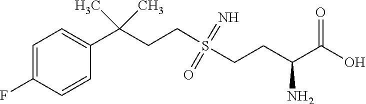 [SMILES: CC(C)(CCS(=N)(=O)CC[C@H](N)C(=O)O)c1ccc(F)cc1] | ++ | @ |
| 59 | 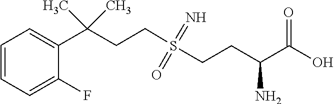 [SMILES: CC(C)(CCS(=N)(=O)CC[C@H](N)C(=O)O)c1ccccc1F] | +++ | ! |
| 60 | 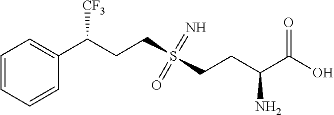 [SMILES: N=[S@](=O)(CC[C@H](c1ccccc1)C(F)(F)F)CC[C@H](N)C(=O)O] | +++ | ! |
| 61 | 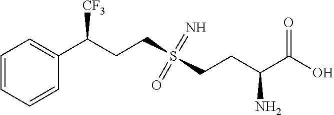 [SMILES: N=[S@@](=O)(CC[C@H](N)C(=O)O)CC[C@@H](c1ccccc1)C(F)(F)F] | +++ | ! |
| 62 | 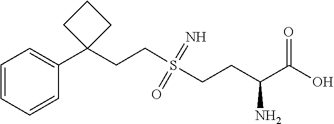 [SMILES: N=S(=O)(CC[C@H](N)C(=O)O)CCC1(c2ccccc2)CCC1] | +++ | ! |
| 63 | 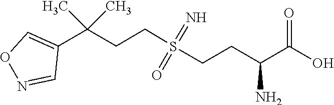 [SMILES: CC(C)(CCS(=N)(=O)CC[C@H](N)C(=O)O)c1cnoc1] | ++ | @ |
| 64 | 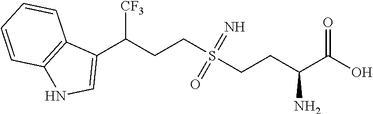 [SMILES: N=S(=O)(CCC(c1c[nH]c2ccccc12)C(F)(F)F)CC[C@H](N)C(=O)O] | +++ | ! |
| 65 | 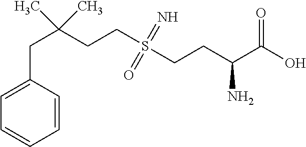 [SMILES: CC(C)(CCS(=N)(=O)CC[C@H](N)C(=O)O)Cc1ccccc1] | +++ | @ |
| 66 | 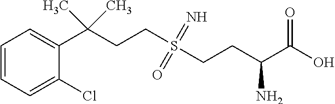 [SMILES: CC(C)(CCS(=N)(=O)CC[C@H](N)C(=O)O)c1ccccc1Cl] | ++ | @ |
| 67 | 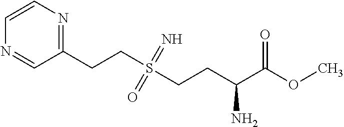 [SMILES: COC(=O)[C@@H](N)CCS(=N)(=O)CCc1cnccn1] | + | @ |
| 68 | 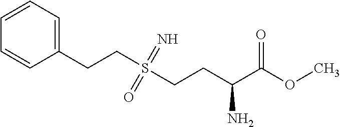 [SMILES: COC(=O)[C@@H](N)CCS(=N)(=O)CCc1ccccc1] | ++ | @ |
| 69 | 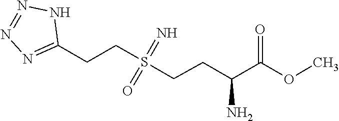 [SMILES: COC(=O)[C@@H](N)CCS(=N)(=O)CCc1nnn[nH]1] | + | @ |
| 70 | 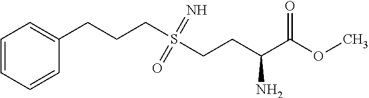 [SMILES: COC(=O)[C@@H](N)CCS(=N)(=O)CCCc1ccccc1] | ++ | @ |
| 71 | 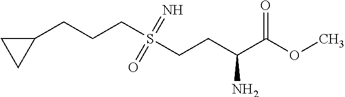 [SMILES: COC(=O)[C@@H](N)CCS(=N)(=O)CCCC1CC1] | ++ | @ |
| 72 | 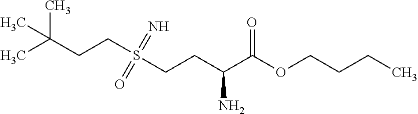 [SMILES: CCCCOC(=O)[C@@H](N)CCS(=N)(=O)CCC(C)(C)C] | ++ | ! |
| 73 | 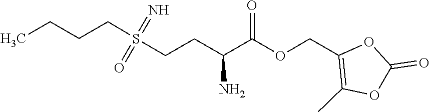 [SMILES: CCCCS(=N)(=O)CC[C@H](N)C(=O)OCc1oc(=O)oc1C] | +, ++ (>10, 0.0774) | @ |
| 74 | 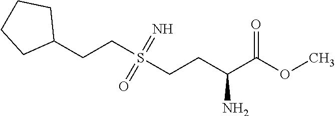 [SMILES: COC(=O)[C@@H](N)CCS(=N)(=O)CCC1CCCC1] | ++ | ! |
| 75 | 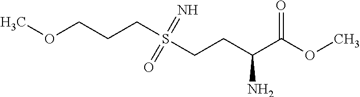 | +, ++ (2.35, 0.594) | @ |
| 76 | 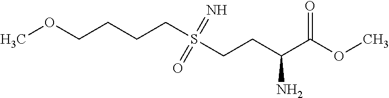 | ++ | @ |
| 77 | 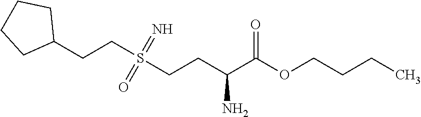 [SMILES: CCCCOC(=O)[C@@H](N)CCS(=N)(=O)CCC1CCCC1] | + | ! |
| 78 | 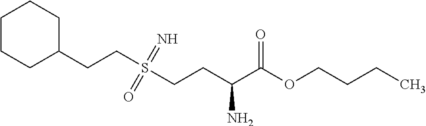 [SMILES: CCCCOC(=O)[C@@H](N)CCS(=N)(=O)CCC1CCCCC1] | ++ | ! |
| 79 | 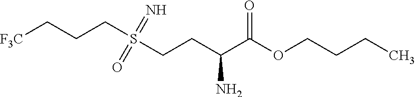 [SMILES: CCCCOC(=O)[C@@H](N)CCS(=N)(=O)CCCC(F)(F)F] | + | ! |
| 80 | 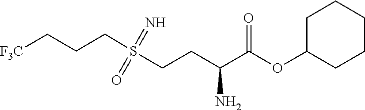 [SMILES: N=S(=O)(CCCC(F)(F)F)CC[C@H](N)C(=O)OC1CCCCC1] | ++ | ! |
| 81 | 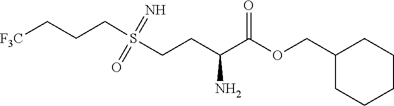 [SMILES: N=S(=O)(CCCC(F)(F)F)CC[C@H](N)C(=O)OCC1CCCCC1] | ++ | ! |
| 82 | 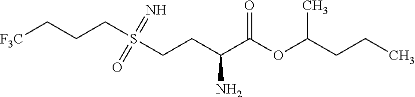 [SMILES: CCCC(C)OC(=O)[C@@H](N)CCS(=N)(=O)CCCC(F)(F)F] | ++ | ! |
| 83 | 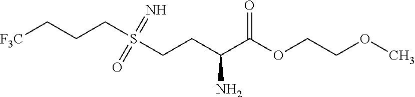 [SMILES: COCCOC(=O)[C@@H](N)CCS(=N)(=O)CCCC(F)(F)F] | ++ | ! |
| 84 | 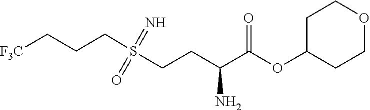 [SMILES: N=S(=O)(CCCC(F)(F)F)CC[C@H](N)C(=O)OC1CCOCC1] | ++ | ! |
| 85 | 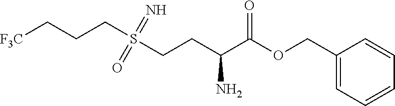 [SMILES: N=S(=O)(CCCC(F)(F)F)CC[C@H](N)C(=O)OCc1ccccc1] | ++ | ! |
| 86 | 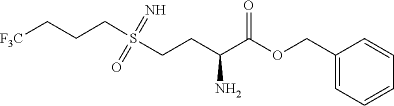 [SMILES: N=S(=O)(CCCC(F)(F)F)CC[C@H](N)C(=O)OCc1ccccc1] | ++ | ! |
| 87 | 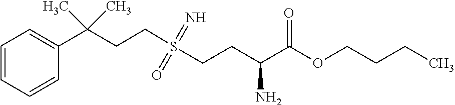 [SMILES: CCCCOC(=O)[C@@H](N)CCS(=N)(=O)CCC(C)(C)c1ccccc1] | ++ | ! |
| 88 | 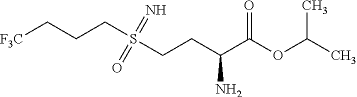 [SMILES: CC(C)OC(=O)[C@@H](N)CCS(=N)(=O)CCCC(F)(F)F] | ++ | ! |
| 89 | 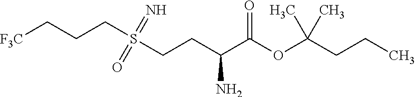 [SMILES: CCCC(C)(C)OC(=O)[C@@H](N)CCS(=N)(=O)CCCC(F)(F)F] | + | ! |
| 90 | 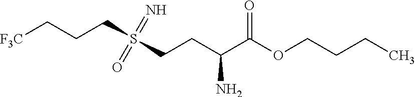 [SMILES: CCCCOC(=O)[C@@H](N)CC[S@@](=N)(=O)CCCC(F)(F)F] | ++ | ! |
| 91 | 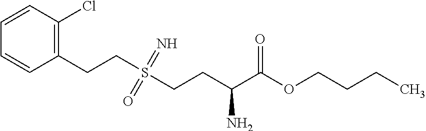 [SMILES: CCCCOC(=O)[C@@H](N)CCS(=N)(=O)CCc1ccccc1Cl] | ++ | ! |
| 92 | 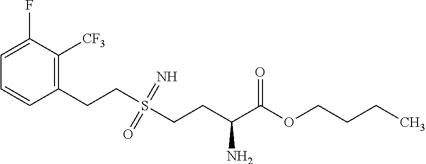 [SMILES: CCCCOC(=O)[C@@H](N)CCS(=N)(=O)CCc1cccc(F)c1C(F)(F)F] | ++ | ! |
| 93 | 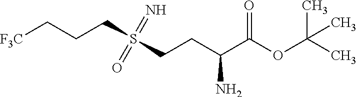 [SMILES: CC(C)(C)OC(=O)[C@@H](N)CC[S@@](=N)(=O)CCCC(F)(F)F] | ++ | ! |
| 94 | 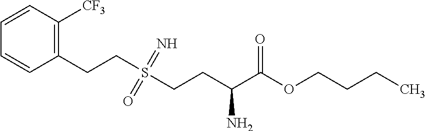 [SMILES: CCCCOC(=O)[C@@H](N)CCS(=N)(=O)CCc1ccccc1C(F)(F)F] | ++ | ! |
| 95 | 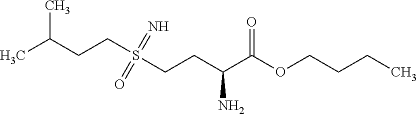 [SMILES: CCCCOC(=O)[C@@H](N)CCS(=N)(=O)CCC(C)C] | ++ | ! |
| 96 | 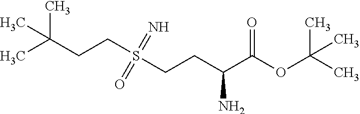 [SMILES: CC(C)(C)CCS(=N)(=O)CC[C@H](N)C(=O)OC(C)(C)C] | + | @ |
| 97 | 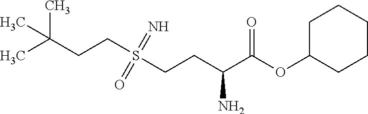 [SMILES: CC(C)(C)CCS(=N)(=O)CC[C@H](N)C(=O)OC1CCCCC1] | + | ! |
| 98 | 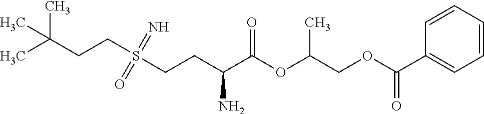 [SMILES: CC(COC(=O)c1ccccc1)OC(=O)[C@@H](N)CCS(=N)(=O)CCC(C)(C)C] | ++ | nd |
| 99 | 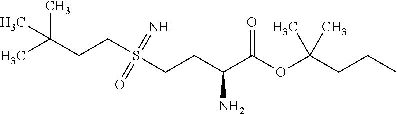 [SMILES: CCCC(C)(C)OC(=O)[C@@H](N)CCS(=N)(=O)CCC(C)(C)C] | + | ! |
| 100 | 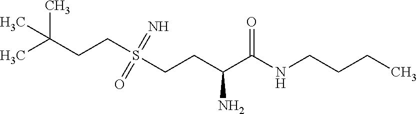 [SMILES: CCCCNC(=O)[C@@H](N)CCS(=N)(=O)CCC(C)(C)C] | + | ! |
| 101 | 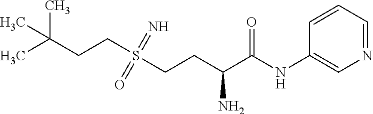 [SMILES: CC(C)(C)CCS(=N)(=O)CC[C@H](N)C(=O)Nc1cccnc1] | + | ! |
| 102 | 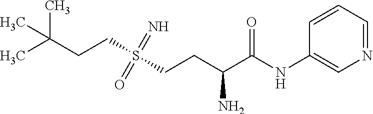 [SMILES: CC(C)(C)CC[S@@](=N)(=O)CC[C@H](N)C(=O)Nc1cccnc1] | + | @ |
| 103 | 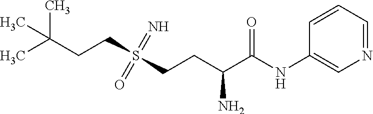 [SMILES: CC(C)(C)CC[S@](=N)(=O)CC[C@H](N)C(=O)Nc1cccnc1] | + | ! |
| 104 | 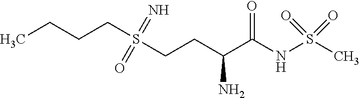 [SMILES: CCCCS(=N)(=O)CC[C@H](N)C(=O)NS(C)(=O)=O] | ++ | @ |
| 105 | 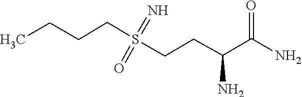 | + | @ |
| 106 | 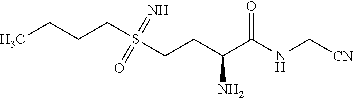 [SMILES: CCCCS(=N)(=O)CC[C@H](N)C(=O)NCC#N] | + | @ |
| 107 | 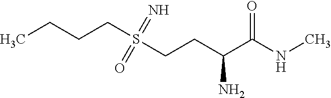 | +, ++ (4.23, 1.96) | @ |
| 108 | 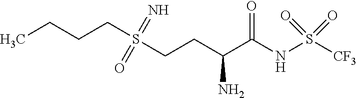 [SMILES: CCCCS(=N)(=O)CC[C@H](N)C(=O)NS(=O)(=O)C(F)(F)F] | + | @ |
| 109 | 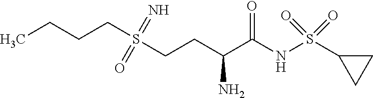 [SMILES: CCCCS(=N)(=O)CC[C@H](N)C(=O)NS(=O)(=O)C1CC1] | ++ | @ |
| 110 | 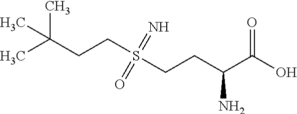 [SMILES: CC(C)(C)CCS(=N)(=O)CC[C@H](N)C(=O)O] | ++ | ! |
| 111 | 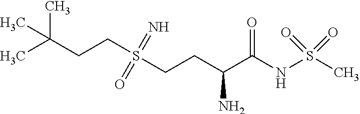 [SMILES: CC(C)(C)CCS(=N)(=O)CC[C@H](N)C(=O)NS(C)(=O)=O] | ++ | @ |
| 112 | 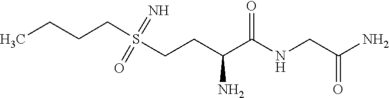 [SMILES: CCCCS(=N)(=O)CC[C@H](N)C(=O)NCC(N)=O] | ++ | @ |
| 113 | 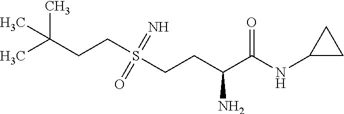 [SMILES: CC(C)(C)CCS(=N)(=O)CC[C@H](N)C(=O)NC1CC1] | ++ | ! |
| 114 | 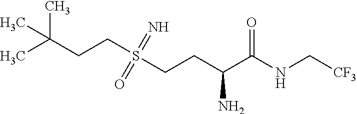 [SMILES: CC(C)(C)CCS(=N)(=O)CC[C@H](N)C(=O)NCC(F)(F)F] | + | ! |
| 115 | 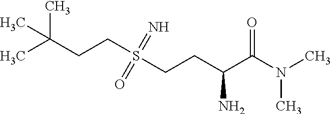 [SMILES: CN(C)C(=O)[C@@H](N)CCS(=N)(=O)CCC(C)(C)C] | + | @ |
| 116 | 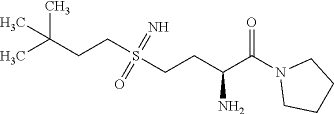 [SMILES: CC(C)(C)CCS(=N)(=O)CC[C@H](N)C(=O)N1CCCC1] | + | @ |
| 117 | 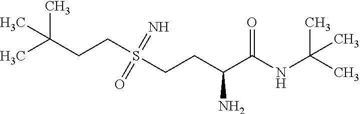 [SMILES: CC(C)(C)CCS(=N)(=O)CC[C@H](N)C(=O)NC(C)(C)C] | + | @ |
| 118 | 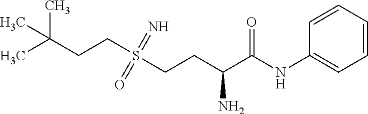 [SMILES: CC(C)(C)CCS(=N)(=O)CC[C@H](N)C(=O)Nc1ccccc1] | + | ! |
| 119 | 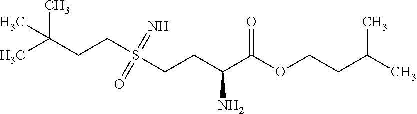 [SMILES: CC(C)CCOC(=O)[C@@H](N)CCS(=N)(=O)CCC(C)(C)C] | + | ! |
| 120 | 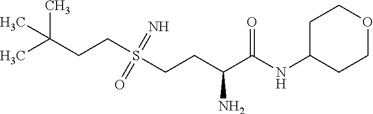 [SMILES: CC(C)(C)CCS(=N)(=O)CC[C@H](N)C(=O)NC1CCOCC1] | + | ! |
| 121 | 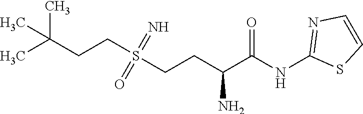 [SMILES: CC(C)(C)CCS(=N)(=O)CC[C@H](N)C(=O)Nc1nccs1] | + | @ |
| 122 | 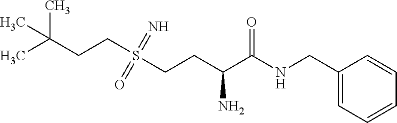 [SMILES: CC(C)(C)CCS(=N)(=O)CC[C@H](N)C(=O)NCc1ccccc1] | + | ! |
| 123 | 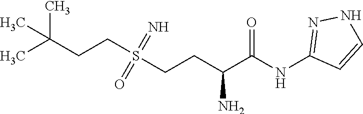 [SMILES: CC(C)(C)CCS(=N)(=O)CC[C@H](N)C(=O)Nc1cc[nH]n1] | +, ++ (>10.0, 1.16) | @ |
| 124 | 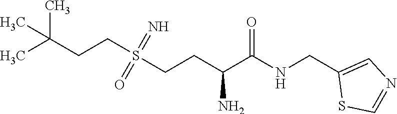 [SMILES: CC(C)(C)CCS(=N)(=O)CC[C@H](N)C(=O)NCc1cncs1] | + | ! |
| 125 | 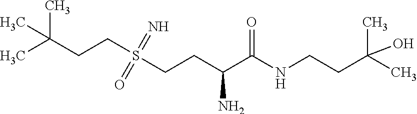 [SMILES: CC(C)(C)CCS(=N)(=O)CC[C@H](N)C(=O)NCCC(C)(C)O] | + | ! |
| 126 | 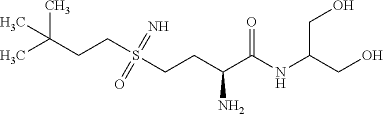 [SMILES: CC(C)(C)CCS(=N)(=O)CC[C@H](N)C(=O)NC(CO)CO] | + | @ |
| 127 | 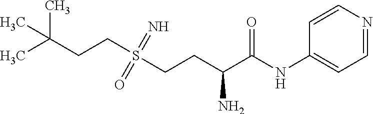 [SMILES: CC(C)(C)CCS(=N)(=O)CC[C@H](N)C(=O)Nc1ccncc1] | + | ! |
| 128 | 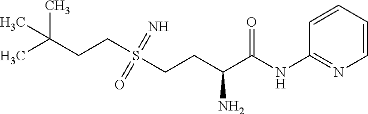 [SMILES: CC(C)(C)CCS(=N)(=O)CC[C@H](N)C(=O)Nc1ccccn1] | ++ | ! |
| 129 | 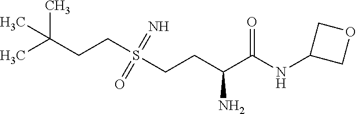 [SMILES: CC(C)(C)CCS(=N)(=O)CC[C@H](N)C(=O)NC1COC1] | + | ! |
| 130 | 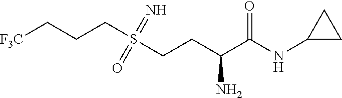 [SMILES: N=S(=O)(CCCC(F)(F)F)CC[C@H](N)C(=O)NC1CC1] | +, ++ (>10, 1.73) | ! |
| 131 | 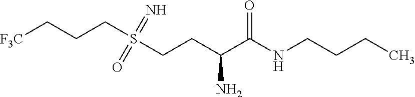 [SMILES: CCCCNC(=O)[C@@H](N)CCS(=N)(=O)CCCC(F)(F)F] | + | ! |
| 132 | 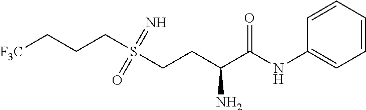 [SMILES: N=S(=O)(CCCC(F)(F)F)CC[C@H](N)C(=O)Nc1ccccc1] | + | ! |
| 133 | 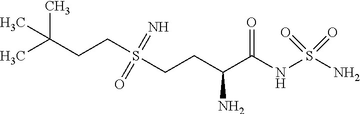 [SMILES: CC(C)(C)CCS(=N)(=O)CC[C@H](N)C(=O)NS(N)(=O)=O] | + | @ |
| 134 | 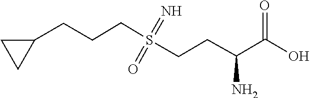 [SMILES: N=S(=O)(CCCC1CC1)CC[C@H](N)C(=O)O] | ++ | @ |
| 135 | 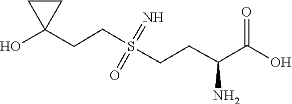 [SMILES: N=S(=O)(CC[C@H](N)C(=O)O)CCC1(O)CC1] | ++ | ! |
| 136 | 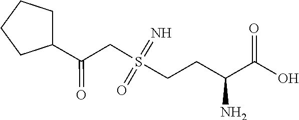 [SMILES: N=S(=O)(CC[C@H](N)C(=O)O)CC(=O)C1CCCC1] | ++ | @ |
| 137 | 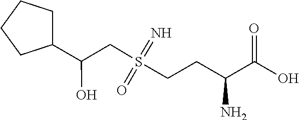 [SMILES: N=S(=O)(CC[C@H](N)C(=O)O)CC(O)C1CCCC1] | ++ | !, @ (1.47, 0.92) |
| 138 | 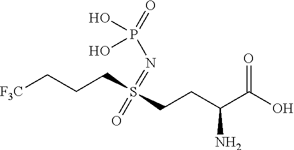 [SMILES: N[C@@H](CC[S@@](=O)(CCCC(F)(F)F)=NP(=O)(O)O)C(=O)O] | nd | ! |
| 139 | 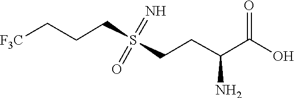 [SMILES: N=[S@](=O)(CCCC(F)(F)F)CC[C@H](N)C(=O)O] | +++ | ! |
| 140 | 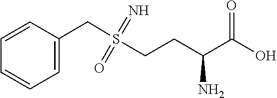 [SMILES: N=S(=O)(CC[C@H](N)C(=O)O)Cc1ccccc1] | + | @ |
| 141 | 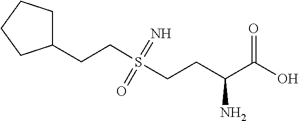 [SMILES: N=S(=O)(CCC1CCCC1)CC[C@H](N)C(=O)O] | +++ | ! |
| 142 | 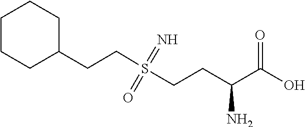 [SMILES: N=S(=O)(CCC1CCCCC1)CC[C@H](N)C(=O)O] | +++ | @ |
| 143 | 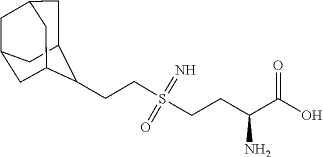 [SMILES: N=S(=O)(CCC1C2CC3CC(C2)CC1C3)CC[C@H](N)C(=O)O] | ++ | @ |
| 144 | 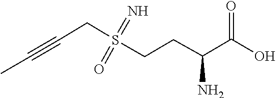 | ++ | @ |
| 145 | 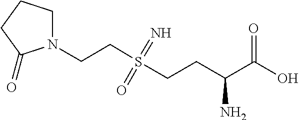 [SMILES: N=S(=O)(CC[C@H](N)C(=O)O)CCN1CCCC1=O] | + | @ |
| 146 | 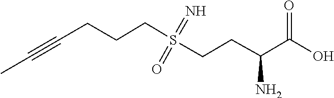 [SMILES: CC#CCCCS(=N)(=O)CC[C@H](N)C(=O)O] | ++ | @ |
| 147 | 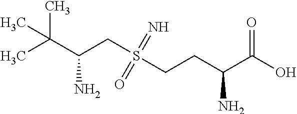 [SMILES: CC(C)(C)[C@@H](N)CS(=N)(=O)CC[C@H](N)C(=O)O] | + | @ |
| 148 | 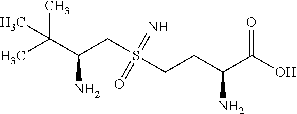 [SMILES: CC(C)(C)[C@H](N)CS(=N)(=O)CC[C@H](N)C(=O)O] | ++ | @ |
| 149 | 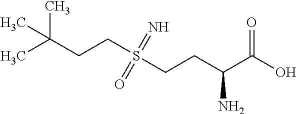 [SMILES: CC(C)(C)CCS(=N)(=O)CC[C@H](N)C(=O)O] | ++ | @ |
| 150 | 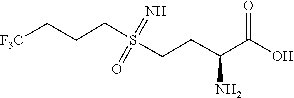 [SMILES: N=S(=O)(CCCC(F)(F)F)CC[C@H](N)C(=O)O] | +++ | ! |
| 151 | 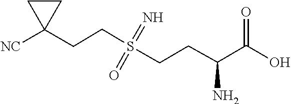 [SMILES: N#CC1(CCS(=N)(=O)CC[C@H](N)C(=O)O)CC1] | ++ | @ |
| 152 | 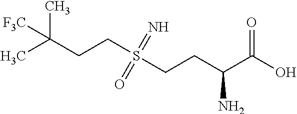 [SMILES: CC(C)(CCS(=N)(=O)CC[C@H](N)C(=O)O)C(F)(F)F] | ++ | ! |
| 153 | 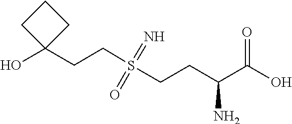 [SMILES: N=S(=O)(CC[C@H](N)C(=O)O)CCC1(O)CCC1] | ++ | ! |
| 154 | 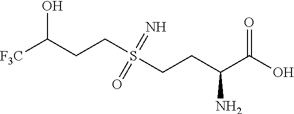 [SMILES: N=S(=O)(CCC(O)C(F)(F)F)CC[C@H](N)C(=O)O] | +++ | ! |
| 155 | 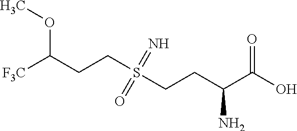 [SMILES: COC(CCS(=N)(=O)CC[C@H](N)C(=O)O)C(F)(F)F] | +++ | ! |
| 156 | 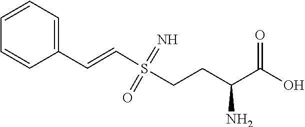 [SMILES: N=S(=O)(/C=C/c1ccccc1)CC[C@H](N)C(=O)O] | ++ | @ |
| 157 | 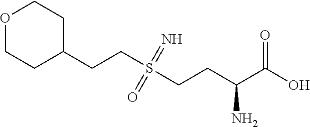 [SMILES: N=S(=O)(CCC1CCOCC1)CC[C@H](N)C(=O)O] | ++ | @ |
| 158 | 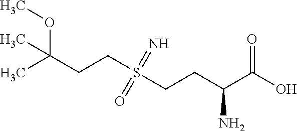 [SMILES: COC(C)(C)CCS(=N)(=O)CC[C@H](N)C(=O)O] | ++ | ! |
| 159 | 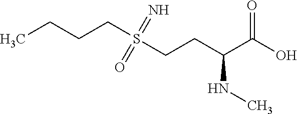 | + | @ |
| 160 | 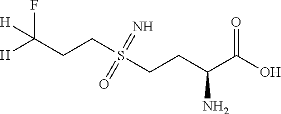 [SMILES: N=S(=O)(CCCF)CC[C@H](N)C(=O)O] | ++ | @ |
| 161 | 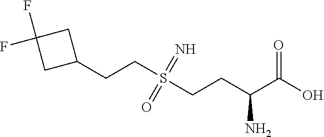 | ++ | ! |
| 162 | 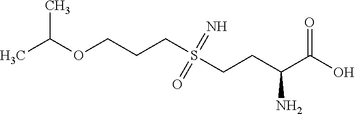 [SMILES: CC(C)OCCCS(=N)(=O)CC[C@H](N)C(=O)O] | ++ | @ |
| 163 | 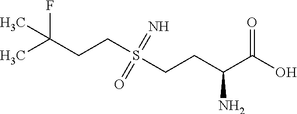 [SMILES: CC(C)(F)CCS(=N)(=O)CC[C@H](N)C(=O)O] | ++ | ! |
| 164 | 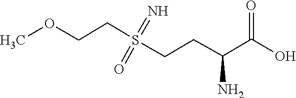 | ++ | @ |
| 165 | 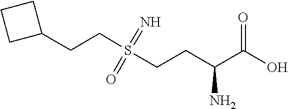 [SMILES: N=S(=O)(CCC1CCC1)CC[C@H](N)C(=O)O] | +++ | ! |
| 166 | 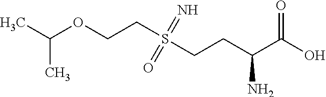 [SMILES: CC(C)OCCS(=N)(=O)CC[C@H](N)C(=O)O] | ++ | @ |
| 167 | 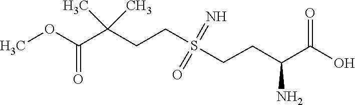 [SMILES: COC(=O)C(C)(C)CCS(=N)(=O)CC[C@H](N)C(=O)O] | ++ | ! |
| 168 | 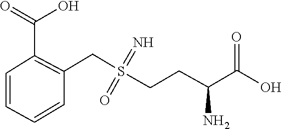 [SMILES: N=S(=O)(CC[C@H](N)C(=O)O)Cc1ccccc1C(=O)O] | ++ | @ |
| 169 | 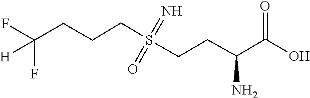 [SMILES: N=S(=O)(CCCC(F)F)CC[C@H](N)C(=O)O] | +++, ++ (0.0102, 0.1) | ! |
| 170 | 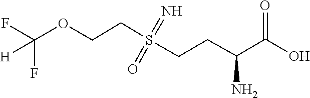 [SMILES: N=S(=O)(CCOC(F)F)CC[C@H](N)C(=O)O] | ++ | @ |
| 171 | 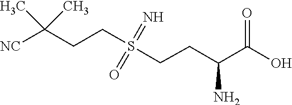 [SMILES: CC(C)(C#N)CCS(=N)(=O)CC[C@H](N)C(=O)O] | ++ | @ |
| 172 | 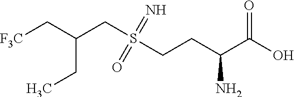 [SMILES: CCC(CC(F)(F)F)CS(=N)(=O)CC[C@H](N)C(=O)O] | ++ | @ |
| 173 | 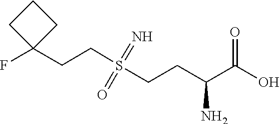 [SMILES: N=S(=O)(CC[C@H](N)C(=O)O)CCC1(F)CCC1] | ++ | ! |
| 174 | 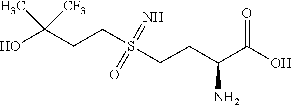 [SMILES: CC(O)(CCS(=N)(=O)CC[C@H](N)C(=O)O)C(F)(F)F] | ++ | ! |
| 175 | 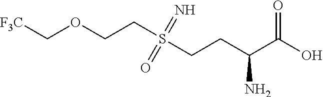 [SMILES: N=S(=O)(CCOCC(F)(F)F)CC[C@H](N)C(=O)O] | ++ | @ |
| 176 | 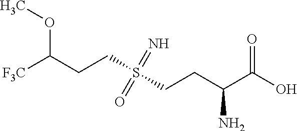 [SMILES: COC(CC[S@@](=N)(=O)CC[C@H](N)C(=O)O)C(F)(F)F] | + | @ |
| 177 | 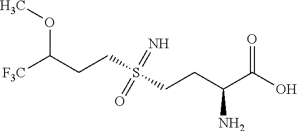 [SMILES: COC(CC[S@@](=N)(=O)CC[C@H](N)C(=O)O)C(F)(F)F] | + | @ |
| 178 | 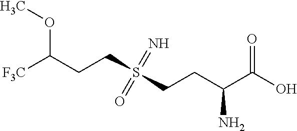 [SMILES: COC(CC[S@](=N)(=O)CC[C@H](N)C(=O)O)C(F)(F)F] | +++ | ! |
| 179 | 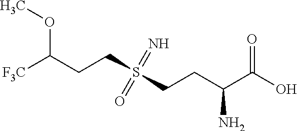 [SMILES: COC(CC[S@](=N)(=O)CC[C@H](N)C(=O)O)C(F)(F)F] | +++ | ! |
| 180 | 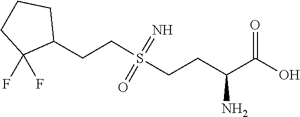 [SMILES: N=S(=O)(CCC1CCCC1(F)F)CC[C@H](N)C(=O)O] | +++ | @ |
| 181 | 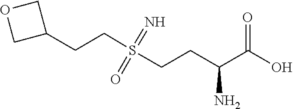 [SMILES: N=S(=O)(CCC1COC1)CC[C@H](N)C(=O)O] | ++ | @ |
| 182 | 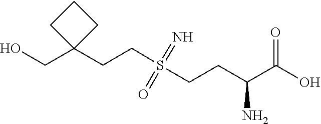 [SMILES: N=S(=O)(CC[C@H](N)C(=O)O)CCC1(CO)CCC1] | ++ | ! |
| 183 | 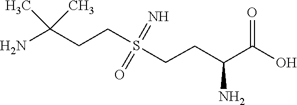 [SMILES: CC(C)(N)CCS(=N)(=O)CC[C@H](N)C(=O)O] | + | @ |
| 184 | 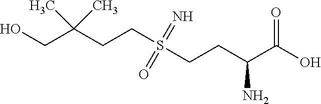 [SMILES: CC(C)(CO)CCS(=N)(=O)CC[C@H](N)C(=O)O] | ++ | ! |
| 185 | 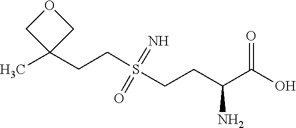 [SMILES: CC1(CCS(=N)(=O)CC[C@H](N)C(=O)O)COC1] | ++ | @ |
| 186 | 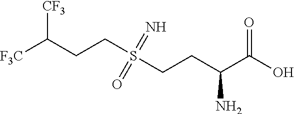 [SMILES: N=S(=O)(CCC(C(F)(F)F)C(F)(F)F)CC[C@H](N)C(=O)O] | +++, ++ (0.022, 0.0183) | ! |
| 187 | 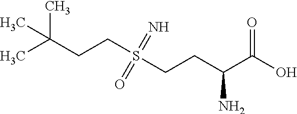 [SMILES: CC(C)(C)CCS(=N)(=O)CC[C@H](N)C(=O)O] | ++ | ! |
| 188 | 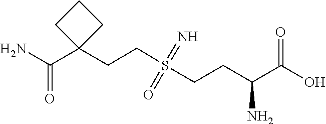 [SMILES: N=S(=O)(CC[C@H](N)C(=O)O)CCC1(C(N)=O)CCC1] | ++ | ! |
| 189 | 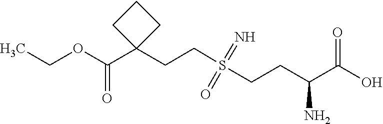 [SMILES: CCOC(=O)C1(CCS(=N)(=O)CC[C@H](N)C(=O)O)CCC1] | ++ | ! |
| 190 | 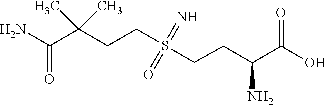 [SMILES: CC(C)(CCS(=N)(=O)CC[C@H](N)C(=O)O)C(N)=O] | ++ | @ |
| 191 | 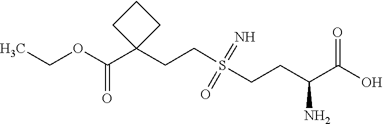 [SMILES: CCOC(=O)C1(CCS(=N)(=O)CC[C@H](N)C(=O)O)CCC1] | ++ | ! |
| 191 | 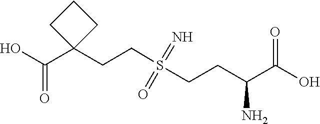 [SMILES: N=S(=O)(CC[C@H](N)C(=O)O)CCC1(C(=O)O)CCC1] | ++ | ! |
| 192 | 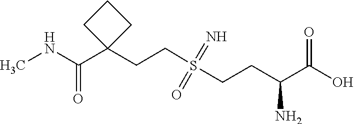 [SMILES: CNC(=O)C1(CCS(=N)(=O)CC[C@H](N)C(=O)O)CCC1] | ++ | @ |
| 193 | 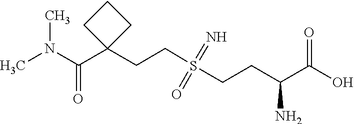 [SMILES: CN(C)C(=O)C1(CCS(=N)(=O)CC[C@H](N)C(=O)O)CCC1] | ++ | @ |
| 194 | 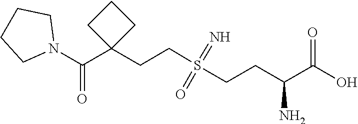 [SMILES: N=S(=O)(CC[C@H](N)C(=O)O)CCC1(C(=O)N2CCCC2)CCC1] | ++ | @ |
| 195 | 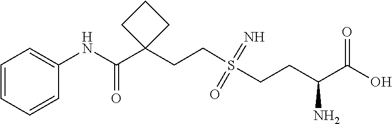 [SMILES: N=S(=O)(CC[C@H](N)C(=O)O)CCC1(C(=O)Nc2ccccc2)CCC1] | ++ | @ |
| 196 | 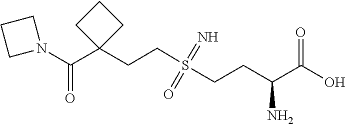 [SMILES: N=S(=O)(CC[C@H](N)C(=O)O)CCC1(C(=O)N2CCC2)CCC1] | ++ | @ |
| 197 | 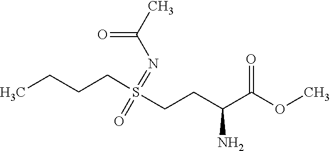 [SMILES: CCCCS(=O)(CC[C@H](N)C(=O)OC)=NC(C)=O] | + | @ |
| 198 | 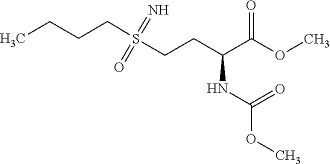 [SMILES: CCCCS(=N)(=O)CC[C@H](NC(=O)OC)C(=O)OC] | + | @ |
| 199 | 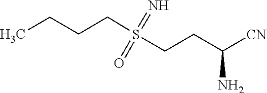 | + | @ |
| 200 | 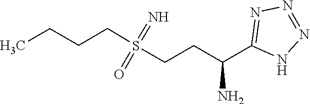 [SMILES: CCCCS(=N)(=O)CC[C@H](N)c1nnn[nH]1] | + | @ |
| 201 | 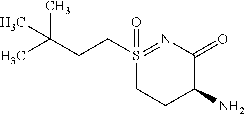 | ++ | ! |
| 202 | 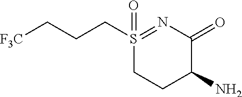 [SMILES: N[C@H]1CCS(=O)(CCCC(F)(F)F)=NC1=O] | ++ | ! |
| 203 | 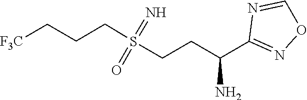 [SMILES: N=S(=O)(CCCC(F)(F)F)CC[C@H](N)c1ncon1] | + | @ |
| 204 | 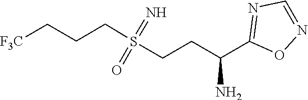 [SMILES: N=S(=O)(CCCC(F)(F)F)CC[C@H](N)c1ncno1] | + | @ |
| 205 | 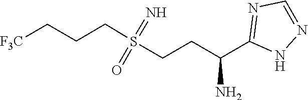 [SMILES: N=S(=O)(CCCC(F)(F)F)CC[C@H](N)c1ncn[nH]1] | +++, ++ (0.206, 0.019) | @ |
| 206 | 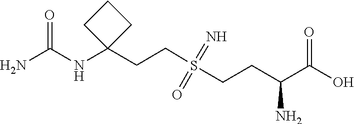 [SMILES: N=S(=O)(CC[C@H](N)C(=O)O)CCC1(NC(N)=O)CCC1] | ++ | ! |
| 207 | 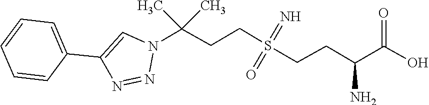 [SMILES: CC(C)(CCS(=N)(=O)CC[C@H](N)C(=O)O)n1cc(-c2ccccc2)nn1] | ++ | @ |
| 208 | 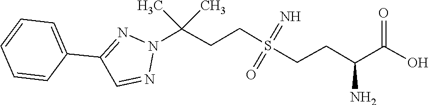 [SMILES: CC(C)(CCS(=N)(=O)CC[C@H](N)C(=O)O)n1ncc(-c2ccccc2)n1] | +++, ++ (0.0204, 0.02) | ! |
| 209 | 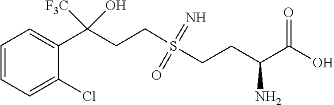 [SMILES: N=S(=O)(CC[C@H](N)C(=O)O)CCC(O)(c1ccccc1Cl)C(F)(F)F] | +++ | ! |
| 210 | 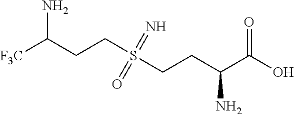 [SMILES: N=S(=O)(CCC(N)C(F)(F)F)CC[C@H](N)C(=O)O] | ++ | ! |
| 211 | 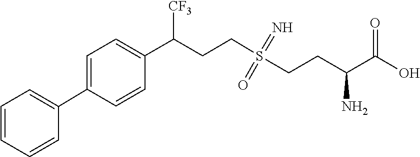 [SMILES: N=S(=O)(CCC(c1ccc(-c2ccccc2)cc1)C(F)(F)F)CC[C@H](N)C(=O)O] | +++ | ! |
| 212 | 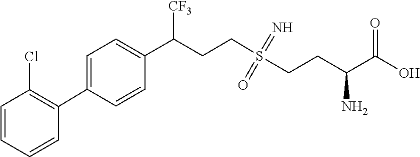 [SMILES: N=S(=O)(CCC(c1ccc(-c2ccccc2Cl)cc1)C(F)(F)F)CC[C@H](N)C(=O)O] | +++ | nd, ! |

TABLE 3
Biological Activity Data Continued

| Compound # | GCL IC₅₀ (mM) | Cell Killing EC₅₀ (mM) |
|---|---|---|
| 213 | +++ | @ |
| 214 | +++ | @ |
| 215 | +++ | ! |
| 216 | ++ | @ |
| 217 | +++ | ! |
| 218 | ++ | @ |
| 219 | + | @ |
| 220 | +++ | ! |
| 221 | +++ | ! |
| 222 | +++ | ! |
| 223 | +++ | ! |
| 224 | +++ | ! |
| 225 | +++ | ! |
| 226 | +++ | ! |
| 227 | +++ | @ |
| 228 | +++ | @ |
| 229 | +++ | @ |
| 230 | +++ | @ |
| 231 | +++ | ! |
| 232 | +++ | ! |
| 233 | +++ | ! |
| 234 | +++ | @ |
| 235 | +++ | ! |
| 236 | +++ | ! |
| 237 | +++ | ! |
| 238 | +++ | ! |
| 239 | +++ | @ |
| 240 | +++ | @ |
| 241 | +++ | @ |
| 242 | +++ | @ |
| 243 | +++ | @ |
| 244 | +++ | @ |
| 245 | +++ | @ |
| 246 | +++ | @ |
| 247 | +++ | @ |
| 248 | +++ | ! |
| 249 | +++ | @ |
| 250 | +++ | @ |
| 251 | +++ | @ |
| 252 | +++ | @ |
| 253 | +++ | ! |
| 254 | +++ | @ |
| 255 | +++ | @ |
| 256 | +++ | ! |
| 257 | +++ | ! |
| 258 | +++ | ! |
| 259 | +++ | @ |
| 260 | +++ | @ |
| 261 | +++ | ! |
| 262 | +++ | ! |
| 263 | +++ | @ |
| 264 | +++ | ! |
| 265 | +++ | ! |
| 266 | +++ | ! |
| 267 | +++ | ! |
| 268 | +++ | ! |
| 269 | +++ | ! |
| 270 | +++ | ! |
| 271 | +++ | ! |
| 272 | +++ | ! |
| 273 | +++ | @ |
| 274 | +++ | @ |
| 275 | +++ | ! |
| 276 | +++ | ! |
| 277 | +++ | ! |
| 278 | +++ | ! |
| 279 | +++ | ! |
| 280 | +++ | ! |
| 281 | +++ | ! |
| 282 | +++ | ! |
| 283 | +++ | @ |
| 284 | +++ | @ |
| 285 | +++ | @ |
| 286 | +++ | @ |
| 287 | +++ | @ |
| 288 | +++ | @ |
| 289 | +++ | @ |
| 290 | +++ | @ |
| 291 | +++ | @ |
| 292 | +++ | @ |
| 293 | +++ | @ |
| 294 | +++ | ! |
| 295 | ++ | @ |
| 296 | +++ | ! |
| 297 | ++ | @ |
| 298 | ++ | @ |
| 299 | ++ | ! |
| 300 | ++ | @ |
| 301 | +++ | ! |
| 302 | ++ | @ |
| 303 | ++ | ! |
| 304 | ++ | nd |
| 305 | +++ | @ |
| 306 | +++ | ! |
| 307 | ++ | ! |
| 308 | ++ | ! |
| 309 | +++ | ! |
| 310 | +++ | ! |
| 311 | +++ | ! |
| 312 | ++ | ! |
| 313 | +++ | ! |
| 314 | +++ | @ |
| 315 | +++ | @ |
| 316 | +++ | ! |
| 317 | +++ | ! |
| 318 | +++ | ! |
| 319 | ++ | ! |
| 320 | +++ | ! |
| 321 | ++ | ! |
| 322 | +++ | ! |
| 323 | + | @ |
| 324 | +++ | ! |
| 325 | + | @ |
| 326 | +++ | ! |
| 327 | + | @ |
| 328 | +++ | ! |
| 329 | + | @ |
| 330 | +++ | @ |
| 331 | +++ | ! |
| 332 | ++ | @ |
| 333 | ++ | @ |
| 334 | +++ | ! |
| 335 | ++ | @ |
| 336 | +++ | @ |
| 337 | +++ | ! |
| 338 | +++ | @ |
| 339 | +++ | ! |
| 340 | +++ | ! |
| 341 | +++ | ! |
| 342 | +++ | ! |
| 343 | ++ | ! |
| 344 | +++ | ! |
| 345 | + | @ |
| 346 | + | @ |
| 347 | +++ | ! |
| 348 | +++ | ! |
| 349 | ++ | ! |
| 350 | +++ | ! |
| 351 | +++ | @ |
| 352 | ++ | ! |
| 353 | +++ | @ |
| 354 | ++ | @ |
| 355 | ++ | @ |
| 356 | ++ | @ |
| 357 | +++ | @ |
| 358 | +++ | ! |
| 359 | ++ | @ |
| 360 | +++ | ! |
| 361 | ++ | @ |
| 362 | ++ | @ |
| 363 | ++ | ! |
| 364 | ++ | @ |
| 365 | ++ | @ |
| 366 | +++ | ! |
| 367 | +++ | ! |
| 368 | ++ | ! |
| 369 | ++ | @ |
| 370 | +++ | ! |
| 371 | +++ | ! |
| 372 | +++ | ! |
| 373 | +++ | ! |
| 374 | ++ | ! |
| 375 | +++ | ! |
| 376 | +++ | ! |
| 377 | +++ | @ |
| 378 | +++ | ! |
| 379 | +++ | ! |
| 380 | +++ | ! |
| 381 | +++ | @ |
| 382 | +++ | @ |
| 383 | +++ | @ |
| 384 | +++ | @ |
| 385 | ++ | ! |
| 386 | +++ | ! |
| 387 | ++ | ! |
| 388 | ++ | ! |
| 389 | + | @ |
| 390 | + | @ |
| 391 | +++ | ! |
| 392 | +++ | ! |
| 393 | +++ | @ |
| 394 | +++ | @ |
| 395 | ++ | ! |
| 396 | ++ | @ |
| 397 | +++ | ! |
| 398 | +++ | ! |
| 399 | +++ | @ |
| 400 | +++ | ! |
| 401 | +++ | @ |
| 402 | +++ | ! |
| 403 | +++ | @ |
| 404 | ++ | ! |
| 405 | ++ | ! |
| 406 | +++ | ! |
| 407 | +++ | @ |
| 408 | +++ | @ |
| 409 | +++ | ! |
| 410 | +++ | @ |
| 411 | ++ | ! |
| 412 | ++ | @ |
| 413 | ++ | @ |
| 414 | ++ | @ |
| 415 | ++ | @ |
| 416 | +++ | @ |
| 417 | ++ | ! |
| 418 | ++ | @ |
| 419 | ++ | @ |
| 420 | +++ | @ |
| 421 | +++ | @ |
| 422 | +++ | ! |
| 423 | +++ | ! |
| 424 | +++ | ! |
| 425 | +++ | @ |
| 426 | ++ | @ |
| 427 | ++ | @ |
| 428 | +++ | ! |
| 429 | +++ | ! |
| 430 | +++ | ! |
| 431 | +++ | ! |
| 432 | +++ | ! |
| 433 | +++ | @ |
| 434 | +++ | ! |
| 435 | +++ | @ |
| 436 | +++ | ! |
| 437 | +++ | ! |
| 438 | +++ | ! |
| 439 | +++ | ! |
| 440 | +++ | ! |
| 441 | ++ | ! |
| 442 | +++ | @ |
| 443 | +++ | ! |
| 444 | +++ | ! |
| 445 | ++ | @ |
| 446 | +++ | ! |
| 447 | +++ | @ |
| 448 | +++ | @ |
| 449 | +++ | @ |
| 450 | +++ | ! |
| 451 | +++ | ! |
| 452 | +++ | @ |
| 453 | +++ | @ |
| 454 | +++ | @ |
| 455 | +++ | @ |
| 456 | +++ | @ |
| 457 | +++ | @ |
| 458 | +++ | ! |
| 459 | +++ | ! |
| 460 | +++ | ! |
| 461 | +++ | ! |
| 462 | +++ | ! |
| 463 | +++ | ! |
| 464 | ++ | @ |
| 465 | +++ | @ |
| 466 | +++ | ! |
| 467 | +++ | ! |
| 468 | +++ | ! |
| 469 | ++ | @ |
| 470 | +++ | @ |
| 471 | +++ | ! |
| 472 | +++ | @ |
| 473 | ++ | ! |
| 474 | +++ | ! |
| 475 | ++ | @ |
| 476 | +++ | ! |
| 477 | +++ | ! |
| 478 | +++ | ! |
| 479 | +++ | ! |
| 480 | ++ | @ |
| 481 | +++ | ! |
| 482 | ++ | @ |
| 483 | ++ | @ |
| 484 | ++ | ! |
| 485 | ++ | ! |
| 486 | ++ | ! |
| 487 | ++ | ! |
| 488 | ++ | ! |
| 489 | + | @ |
| 490 | + | @ |
| 491 | ++ | ! |
| 492 | + | @ |
| 493 | + | nd |
| 494 | +++ | @ |
| 495 | ++ | @ |
| 496 | ++ | ! |
| 497 | ++ | @ |
| 498 | +++ | ! |
| 499 | +++ | @ |
| 500 | +++ | @ |
| 501 | +++ | @ |
| 502 | +++ | ! |
| 503 | ++ | @ |
| 504 | +++ | @ |
| 505 | +++ | @ |
| 506 | ++ | ! |
| 507 | +++ | @ |
| 508 | ++ | @ |
| 509 | +++ | @ |
| 510 | +++ | @ |
| 511 | ++ | ! |
| 512 | + | @ |
| 513 | +++ | @ |
| 514 | ++ | @ |
| 515 | +++ | ! |
| 516 | + | @ |

### (2) Priming Agents

Provided herein are methods of inducing targeted cell death in a mammalian tissue in vivo, the methods comprising: (a) contacting a mammalian tissue with a priming agent; (b) contacting the mammalian tissue in vivo with an effective amount of a ferroptosis-inducing agent for a duration of time of at least 4 hours, when a plurality of cells within the mammalian tissue are responsive to the priming agent as determined by detecting in the mammalian tissue: (i) a plurality of cells comprising a concentration of selenium greater than a selenium concentration in the mammalian tissue prior to contacting with the priming agent; (ii) a plurality of cells comprising a concentration of iron greater than an iron concentration in the mammalian tissue prior to contacting with the priming agent; (iii) a plurality of cells comprising a PUFA concentration greater than a PUFA concentration in the mammalian tissue prior to contacting with the priming agent; (iv) a plurality of cells expressing one or more markers indicative of a mesenchymal state; (v) a plurality of cells comprising a peroxidizability index (PI) greater than a PI in the mammalian tissue prior to contacting with the priming agent; and/or (vi) hyperproliferation of cells in the mammalian tissue, wherein the ferroptosis-inducing agent induces targeted cell death in the mammalian tissue in vivo. In some embodiments, a priming agent is administered prior to the administration of a ferroptosis-inducing agent provided herein. In some embodiments, the priming agent is administered in vivo, in vitro, or ex vivo. A priming agent is an agent that prepares a subject or tissue for administration of a therapeutically effective dose of a ferroptosis-inducing agent provided herein. In some embodiments, the priming agent is a ferroptosis-inhibitor. In some embodiments, the priming agent renders a cell within a tissue as ferroptosis-sensitive. In some embodiments, the priming agent is a lipophilic antioxidant or radical trapping agent. In some embodiments, the priming agent is a polyunsaturated fatty acid. In some embodiments, the priming agent is an iron chelator. In some embodiments, the priming agent is a lipid peroxidation inhibitor. In some embodiments, the priming agent modulates blood oxygen levels. In some embodiments the priming agent is a hydroperoxide. In some embodiments, the priming agent is selected from the group consisting of: liproxstatin-1, ferrostatin-1, deferoxamine (DFO), iron, selenium, vitamin E, erythropoietin, a polyunsaturated fatty acid, N-acetylcysteine, pifithrin-alpha-HBr, and methylnaphthalene-4-propionate endoperoxide (MNPE). In some embodiments, the polyunsaturated fatty acid is selected from the group consisting of: hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, Timnodonic acid), heneicosapentaenoic acid (HPA), docosapentaenoic acid (DPA, Clupanodonic acid), docosahexaenoic acid (DHA, Cervonic acid), tetracosahexaenoic acid (Nisinic acid), tetracosapentaenoic acid, linoleic acid (LA), gamma-linolenic acid (GLA), eicosadienoic acid, dihomo-gamma-linolenic acid (DGLA), arachidonic acid (AA), docosadienoic acid, adrenic acid (AdA), docosapentaenoic acid (Osbond acid), tetracosatetraenoic acid, and tetracosapentaenoic acid. Non-limiting examples of priming agents are provided in Table 4.

TABLE 4
Priming Agents.

| | Chemical | | Target/Mechanism |
|---|---|---|---|
| Name | Formula/CAS | Chemical Structure | of Action |
| liproxstatin-1 | C₁₉H₂₁ClN₄ Cas: 950455-15- 9 | 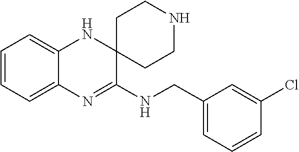 [SMILES: Clc1cccc(CNC2=Nc3ccccc3NC23CCNCC3)c1] | Modulates ferroptosis by inhibiting GCL and GSS |
| ferrostatin-1 | C₁₅H₂₂N₂O₂ Cas: 347174-05- 4 | 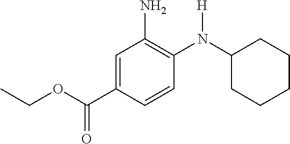 [SMILES: CCOC(=O)c1ccc(NC2CCCCC2)c(N)c1] | Modulates ferroptosis by inhibiting lipid peroxidation |
| deferoxamine mesylate (DFO) | C₂₆H₅₂N₆O₁₁S Cas: 138- 14-7 | 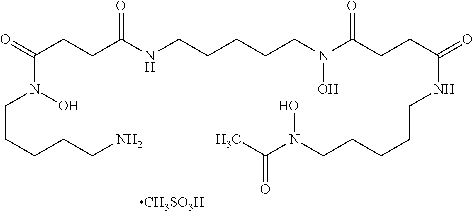 | Modulates ferroptosis by chelating iron |
| iron | Fe | Fe⁺² | Modulates |
| | | | ferroptosis iron- |
| | | | dependent cell |
| | | | death by multiple |
| | | | mechanisms |
| selenium | Se | Se | Modulates |
| | | | ferroptosis by |
| | | | increasing |
| | | | cellular |
| | | | glutathione |
| | | | peroxidase |
| | | | activity and |
| | | | reduces |
| | | | susceptibility to |
| | | | lipid |
| | | | peroxidation |
| Se-Methyl- selenocysteine (hydrochloride) | C₄H₉NO₂Se•HCl Cas: 863394-07- 4 | 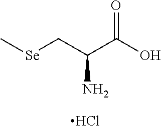 | Modulates ferroptosis by preventing increases in glutathione reductase and glutathione peroxidase |
| | | | (GPX) activity, |
| | | | as well as |
| | | | decreases in |
| | | | glutathione |
| | | | (GSH) levels |
| vitamin E (α- tocopherol) | C₃₁H₅₂O₃ Cas: 7695- 91-2 | 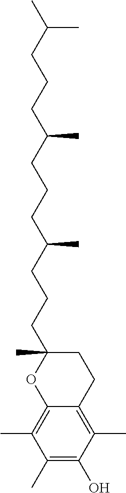 | Modulates ferroptosis by lipoxygenase inhibition |
| α-tocotrienol | C₂₉H₄₄O₂ Cas: 58864- 81-6 | 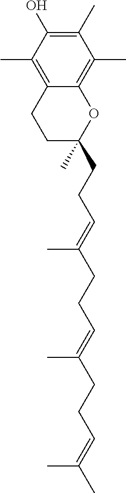 | Modulates ferroptosis by lipoxygenase inhibition |
| Trolox | C₁₄H₁₈O₄ Cas: 53188- 07-1 | 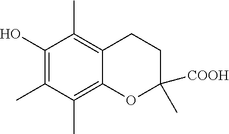 [SMILES: Cc1c(C)c2c(c(C)c1O)CCC(C)(C(=O)O)O2] | Derivative of vitamin E with potent antioxidant properties. Modulates ferroptosis by lipoxygenase inhibition |
| erythropoietin | | erythropoietin precursor [Homo | glycoprotein |
| polypeptide | | sapiens] | cytokine that |
| | | NCBI Reference Sequence: | modulates |
| | | NP_000790.2 | ferroptosis by |
| | | MGVHECPAWLWLLLSLLSLPLGL | increasing |
| | | PVLGAPPRLICDSR VLERYLLEAK | systemic oxygen |
| | | EAENITTGCAEHCSLNENITVPDT | capacity by |
| | | KVNFYAWKRMEVGQQAVEVWQ | stimulating the |
| | | GLALLSEAVLRGQALLVNSSQPW | activity of GPX4 |
| | | EPLQLHVDKAVSGLRSLTTLLRA | and inhibiting |
| | | LGAQKEAISPPDAASAAPLRTITA | lipid peroxides |
| | | DTFRKLFRVYSNFLRGKLKLYTG | |
| | | EACRTGDR (SEQ ID NO: 1). | |
| | | erythropoietin [Homo sapiens] | |
| | | NCBI Reference Sequence: GenBank: | |
| | | AGW15567.1 | |
| | | MGVHECPAWLWLLLSLLSLPLGL | |
| | | PVLGAPPRLICDSR VLERYLLEAK | |
| | | EAENITTGCAEHCSLNENITVPDT | |
| | | KVNFYAWKRMEVGQQAVEVWQ | |
| | | GLALLSEAVLRGQALLVNSSQPW | |
| | | EPLQLHVDKAVSGLRSLTTLLRA | |
| | | LGAQKEAISPPDAASAAPLRTITA | |
| | | DTFRKLFRVYSNFLRGKLKLYTG | |
| | | EACRTGDRV (SEQ ID NO: 2). | |
| N-Acetylcysteine amide | C₅H₁₀N₂O₂S Cas: 38520- 57-9 | 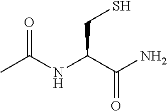 | Modulates ferroptosis by inhibiting glutamate- induced cytotoxicity, decreases in |
| | | | intracellular |
| | | | glutathione |
| | | | (GSH) levels, |
| | | | and increases in |
| | | | intracellular |
| | | | reactive |
| | | | species levels (ROS) |
| | | | levels |
| pifithrin-alpha- HBr | C₁₆H₁₈N₂OS•xHBr Cas: 63208- 82-2 | 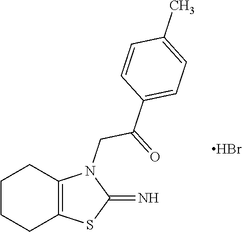 [SMILES: Cc1ccc(C(=O)Cn2c3c(sc2=N)CCCC3)cc1 ; Br] | Modulates cell death and ferroptosis by p53 inhibition |
| RC574 | C₁₈H₂₄OSSe Cas: 2584411- 87-8 | 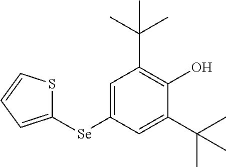 | Increases glutathione peroxidase 1 (GPX1) levels and GPX activity, inhibits ferroptosis induced by GPX4 inhibition |

In some embodiments, methods provided herein comprise administering any one of the agents listed in Table 1, Table 2, Table 3, or Table 4, or any compound, agent, or therapeutic herein enantiomer of any of these, diastereomer of any of these, pharmaceutically acceptable salt of any of these, or deuterated derivative of any of these. Further provided herein are pharmaceutical compositions, wherein the compositions comprise a ferroptosis-inducing agent or compound and a priming agent or an enantiomer or diastereomer of any of the foregoing, a pharmaceutically acceptable salt of any of the foregoing, or a deuterated derivative of any of the foregoing. In some embodiments, the pharmaceutical compositions further comprise a chemotherapeutic agent.

### (3) Additional Treatments and Cell Death-Inducing Agents

In some embodiments, the methods provided herein comprise administering at least one additional treatment to a subject. In some embodiments, the additional treatment is surgery. In some embodiments, the additional treatment is radiation therapy. In some embodiments, the additional treatment is a dietary supplement. Non-limiting examples of dietary supplements include: probiotics, selenium, iron, vitamins (e.g., vitamin A, vitamin C, vitamin E), curcumin, fish oils, beta carotene, hydrogen sulfides, fatty acids, methionine, cysteine, homocysteine, taurine, cystine or di-cysteine. In some embodiments, the dietary supplement is a high-selenium nutritional supplement.

In some embodiments, the additional treatment is an additional therapeutic agent. In some embodiments, the methods provided herein comprise administering an additional agent in combination with a ferroptosis-inducing agent, an iron-dependent cell death inducing agent, and/or a priming agent provided herein. In some embodiments, the additional agent is a cell-death inducing agent. In some embodiments, the additional agent is an anti-cancer agent. In some embodiments, the anti-cancer agent is a chemotherapeutic agent. A chemotherapeutic agent or compound is any agent or compound useful in the treatment of cancer. The chemotherapeutic cancer agents that can be used in combination with ferroptosis-inducing agents or iron-dependent cell death agents provided herein which include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The disclosed agents provided herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the ferroptosis-inducing agents provided herein can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bevacizumab; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can be administered at a clinically effective dose. A chemotherapeutic can also be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of an agent provided herein. In some cases, a subject can have a refractory cancer that is unresponsive to a chemotherapeutic.

### General Methods of Making Pharmaceutical Compounds

The compounds described herein can be provided as amorphous solids or crystalline solids. The compounds of Formula (I) can be provided as amorphous solids or crystalline solids. In some embodiments, the crystalline solid is a pure crystalline solid. In some embodiments, the crystalline solid is a polymorph. A particular polymorph can have distinct pharnaceutically relevant physical properties in comparison with another polymorph. In some embodiments, a polymorph described herein may be characterized by single X-ray diffraction methods. A crystalline form of a compound described herein can be anhydrous, a hydrate, or a solvate. Lyophilization can be employed to provide the compounds as amorphous solids. It should further be understood that solvates (e.g., hydrates) of the compounds are also contemplated herein. The term “solvate” can mean a physical association of a compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. In addition, compounds, subsequent to their preparation, can be isolated and purified to obtain a composition containing an amount by weight equal to or greater than 99% of a compound of Formula (I) (“substantially pure”), which is then used or formulated as described herein. Such “substantially pure” compounds are also contemplated herein. Compounds can be prepared in several ways and can be synthesized using the methods described herein. The reactions and techniques described herein are performed in solvents appropriate to the reagents and materials employed and are suitable for the transformations being affected. Also, in the description of the synthetic methods described below, it is to be understood reaction conditions, including choice of solvent, reaction atmosphere, reaction temperature, duration of the experiment and work up procedures, can be chosen to be the conditions standard for that reaction. It is understood by one skilled in the art of organic synthesis that the functionality present on various portions of the molecule must be compatible with the reagents and reactions proposed.

In some embodiments, schemes included herein comprise structures containing Protecting Groups designated “P”, “P¹”, “P²”, “P³”, etc. A protecting group herein can be, for example, Boc, mesyl (Ms), tosyl (Ts), nosyl (Ns), benzyl (Bn), benzoyl (Bz), SEM, TMS, TIPS, Cbz, or FMOC. These include conventional protecting groups utilized in organic synthesis. In some embodiments, schemes included herein comprise structures containing alkyl, alkenyl, alkynyl, aryl, heteroalkyl, heteroaryl, heteroalkenyl, or heteroalkynyl groups, halogenated derivatives thereof, or combinations thereof designated as “R” groups. In some instances, R can be, for example: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —C(O)OR₅, —C(O)NH₂, —O—, —S—, —OH, —NH₂, —NH—, a halogen, —CF₃, —CN, —F, —Cl, —Br, —I, or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F.

In some embodiments, a compound described in the exemplary Schemes herein is presented with the following stereochemical configuration:

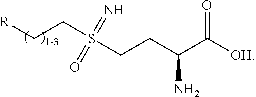

This presentation of the compound is the same as a compound presented as:

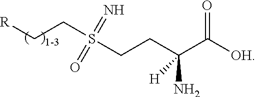

In some embodiments, schemes included herein comprise structures representative of compounds described herein.

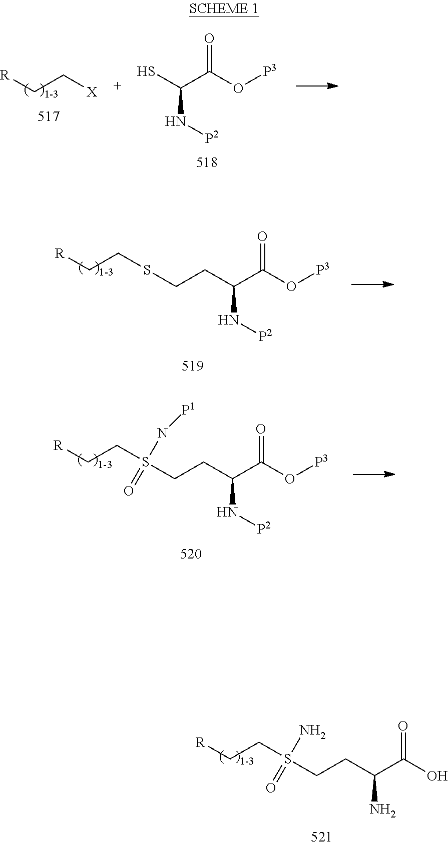

Compounds 521 may be prepared according to the synthetic route outlined in Scheme 1. For example, an appropriately substituted alkyl group (517) with an electrophilic center, such as an alkyl halide or sulfonate, may be reacted with a protected cysteine derivative, such as 518, in the presence of a base to afford the sulfur alkylated product 519. Compounds 519 may be reacted with a variety of oxidants, such as PhI(OAc)₂ and ammonium carbamate to provide the sulfoximine derivative 520 which may or may not have additional substitution on the sulfoximine nitrogen as a protecting group. Compounds 520 may be reacted with reagents, such as NaOH or TFA, to transform the ester to a carboxylic acid and remove the nitrogen protecting group (P2) to afford compounds 521.

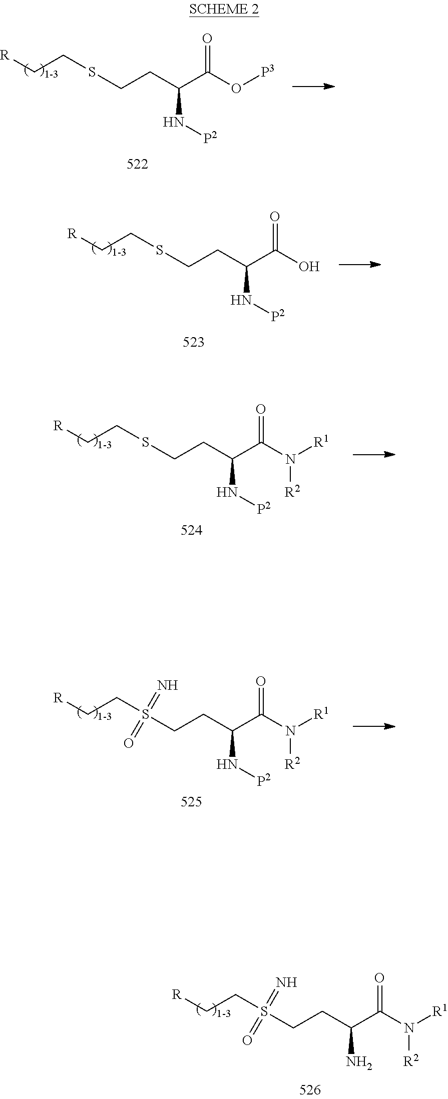

Additional compounds described herein may be prepared according to the general route outlined in Scheme 2. Treating compounds such as 522 with a reagent, such as a strong base or strong acid, may provide the free carboxlic acid 523. Reaction of 523 with a variety of amines in the presence of a suitable amide bond forming reagent, such as PyBOP, may provide the amide 524. The sulfur can be reacted under oxidizing conditions to provide the sulfoximine 525 which may be further reacted with amine protecting group removal reagents, such as HCl in dioxane to provide compounds such as Compound 526.

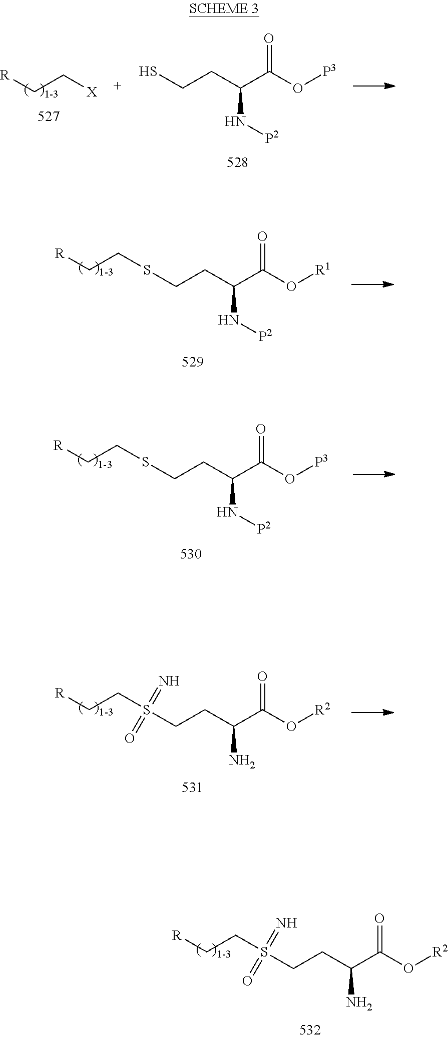

An additional scheme outlining paths to compounds 532 is shown in Scheme 3. Reacting compound 527 with 528 may provide compounds such a 529. Treatment of 529 with suitable transesterification reagents, such as methanol and HCl, can provide compounds such as 530. Alternatively, P³ in compound 529 may be removed to provide P³=hydrogen upon which that compound is alkylated in the presence of base with a variety of alkyl halides followed by a sulfonation reaction to provide 528. Compound 528 may be reacted with a variety of oxidizing reagents to provide the sulfoximine derivative 531 which may be further treated with reagents, such as TFA or HCl, to remove the amine protecting group and provide compounds 532.

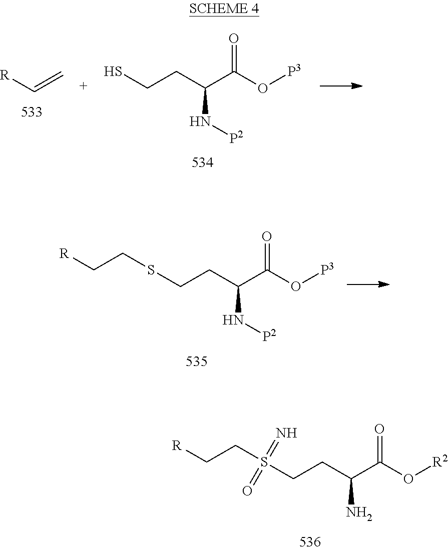

Scheme 4 provides an alternative route to compounds 536. Reacting compound 533 directly with compounds such as 534 in the presence of a radical generating reagent, such as AIBN, may provide the coupled intermediate 535. Compound 535 can similarly follow the sulfur oxidation steps and amine deprotection as previously outlined in Schemes 1-3.

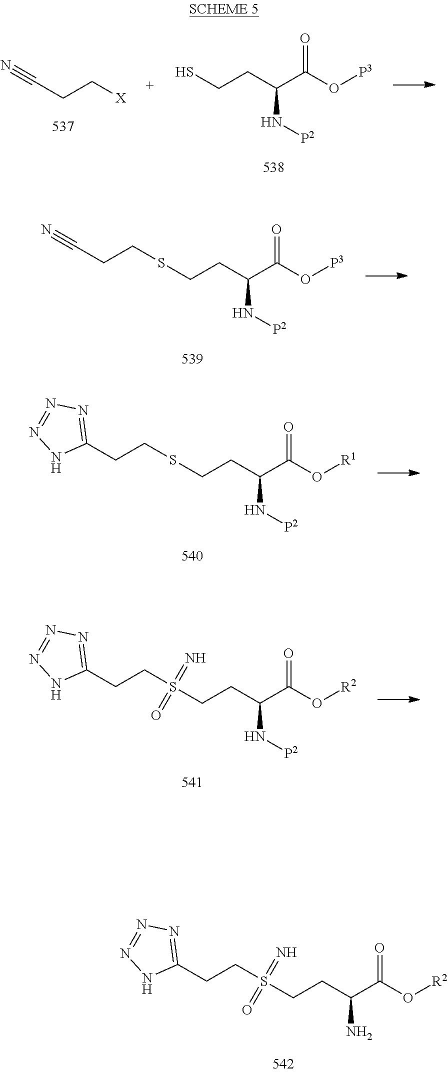

Scheme 5 demonstrates a process for making heterocycle substituted analogs of 542. Reacting compound 357, containing a nitrile, with the appropriately protected sulfur nucleophile 358 may provide the alkylated analog 539. Treatment of 539 with a reagent, such as TMS-azide, may provide the tetrazole substituted intermediate 540. Oxidation of the sulfur to the sulfoximine provides 541 which can be deprotected to the final analog 542.

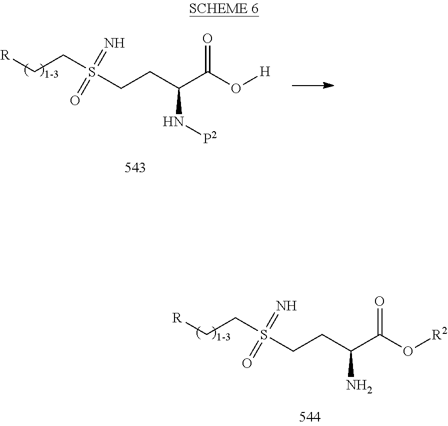

Intermediates containing a free carboxylic acid, such as 543 in Scheme 6, may be esterified under a under acidic conditions, such as methanol in HCl, to provide compounds 544. Alternatively, the carboxylic acid may be alkylated directly with an appropriate electrophile, such as butyl bromide, and a base, such as K₂CO₃ and then further transformed to (I) by deprotection of the amine group under previously described conditions.

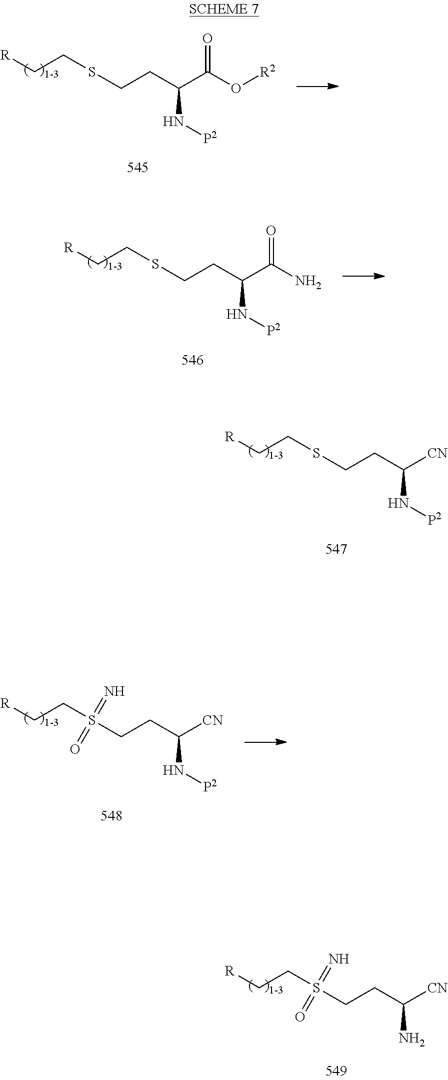

Scheme 7 provides a route for preparing compounds 549. Treatment of ester 545 with a direct amide forming reagent, such as NH₃ in methanol, may provide 546. Compound 546 may be further reacted with a dehydrating reagent, such as trifluoroacetic anhydride, to provide compounds such as 547. Reaction of 547 with oxidants such as PhI(OAc)₂ and ammonium carbamate may provide the sulfoximine 548. Further treatment of 548 with reagents that would remove the amine protecting group may provide compounds 549.

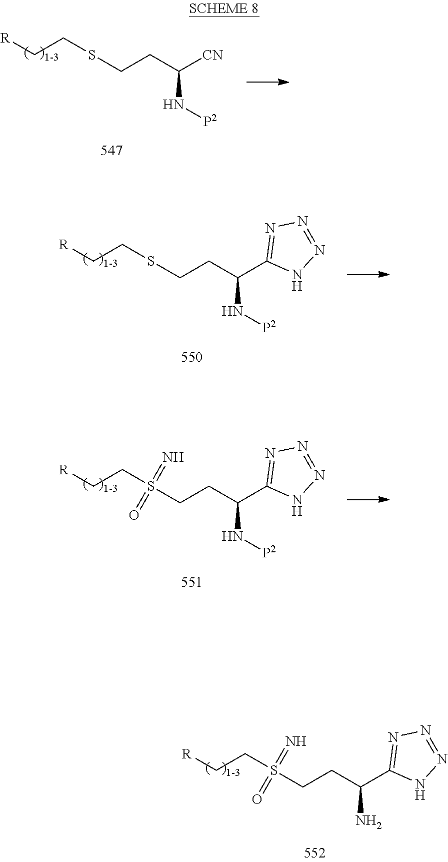

Compounds 552 may be prepared from the previously described intermediate 547. Reaction of 547 with reagents that will transform the nitrile to a tetrazole, such as NaN₃, and ZnBr₂, may provide compounds such as 550. Oxidation of the sulfur to the sulfoximine with PhI(OAc)₂ and ammonium carbamate may provide 551 which can be further reacted with anhydrous acid, such as HCl in dioxane to provide 552.

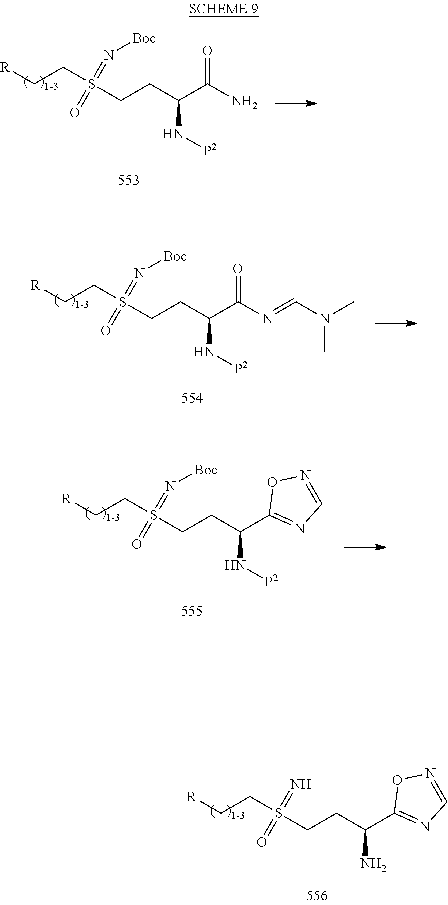

An alternative to compounds 556 is shown in Scheme 9. An appropriately protected primary amide, 553, may be reacted with DMF-DMA to form the amidine 554. Reaction with a bis-heteroatom nucleophile, such as hydroxyl amine, may afford compounds such as 555. Treatment of 555 with acid, such as TFA or HCl to remove the amine protecting groups may provide compounds 556.

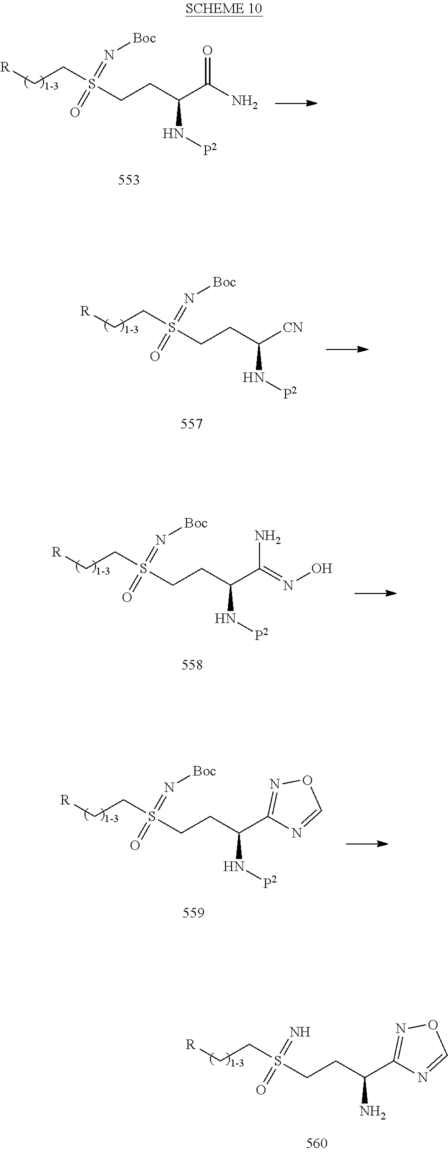

Scheme 10 outlines another possible route to compounds 560. Intermediate 553 may be converted to the nitrile 557 under dehydrating conditions and further reacted with hydroxyl amine to provide intermediates such as 558. Reaction of 558 with a methylene equivalent, such as trimethoxymethane, in the presence of acid may provide compounds such as 559. Further reaction with amine deprotecting group reagents, such as TFA or HCl, may provide compounds 560.

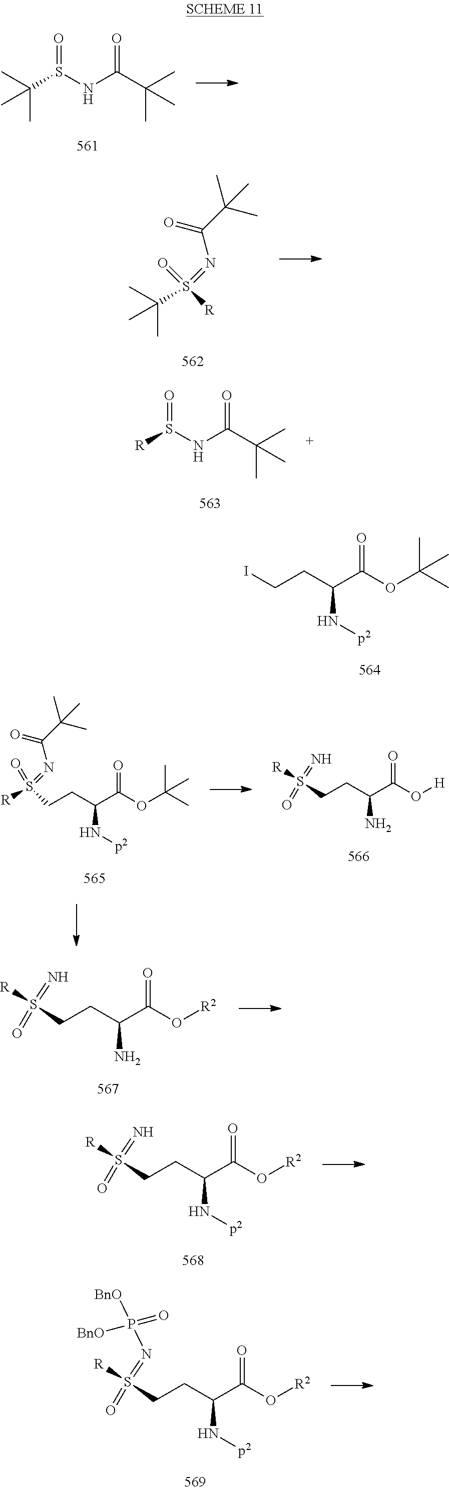

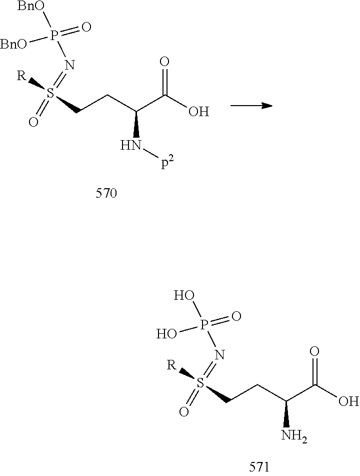

A stereoselective synthesis of compounds 566 is shown in Scheme 11. The chiral sulfinamide 561 may be alkylated on sulfur to provide intermediate 562. Reaction of 562 with an acid, such as TFA, may provide sulfinamide 563. A subsequent alkylation of 563 with functionalized reagents, such as 564, may provide the fully substituted intermediate 565. Reaction of 565 with acids such as TFA or HCl may provide the optically pure 566. Intermediate 565, or compounds of similar structure, may be further transformed to 571 by reacting 565 with selective deprotecting reagents to first afford 567. Protection of the free primary amine (567) to afford 568 allows for the phosphorylation of the sulfoximine nitrogen by reacting with reagents such as dibenzyl phosphate to provide 569. Unmasking of the various protecting groups provides the phosphorylated versions consistent with a compound 571.

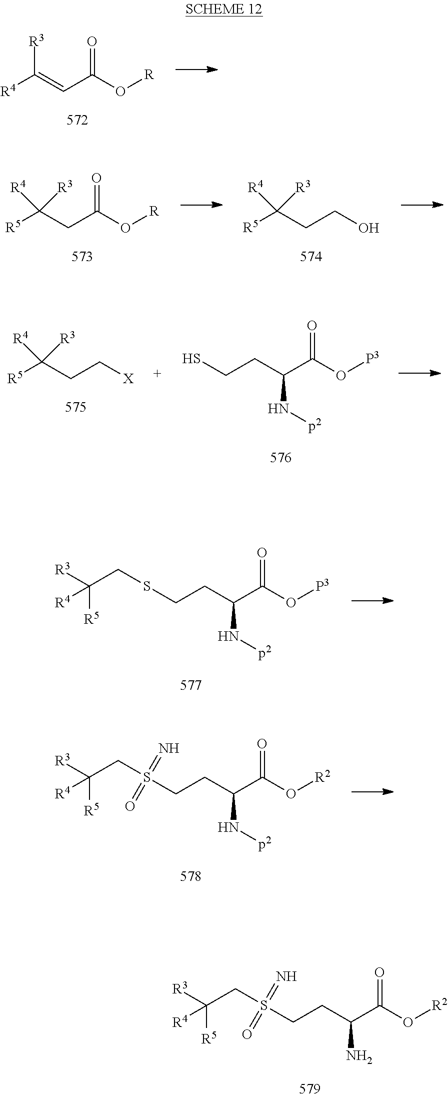

An additional method for the general preparation of compounds of interest in the application is shown in Scheme 12. Substituted olefins, such as 572, may be reacted with a variety of nucleophiles, such as an alkyl cuprate or aryl boronic acid, to provide poly-substituted esters such as 573. Treatment of 573 with standard reducing reagents, such as LAH or NaBH₄, may provide the alcohol 574. Conversion of 574 to an alkylating agent, either as an alkyl halide or alkyl sulfonate, may be achieved under standard conditions. For example, treatment of 574 with MsCl in an appropriate solvent, such as DCM, in the presence of a base, such as DIPEA, may provide compounds such as 575. Compound 575 may be reacted with the sulfur containing reagent 576 in the presence of a base, such as K₂CO₃, to provide 577. Compound 12e may be treated with an oxidizing reagent, such as PhI(OAc)₂ and ammonium carbamate to provide compounds such as 578 which can further be treated with anhydrous acid, such as HCl in dioxane to provide compounds 579.

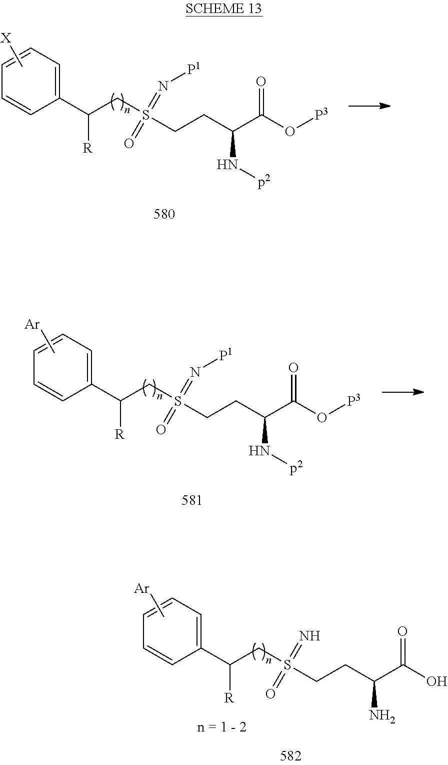

Alternative variations of compounds 582 may be prepared using the methods outlined in Scheme 13. Starting from compounds such as 580, the aryl (or heteroaryl) group may be optionally substituted with groups, such as an iodide) that facilitate cross coupling reactions with reagents such as aryl boronic acids to form compounds of the formula 581. Compound 581 may be reacted with reagents, such as NaOH or TFA, to transform the ester to a carboxylic acid and remove the nitrogen protecting group (P2) to afford compounds 582.

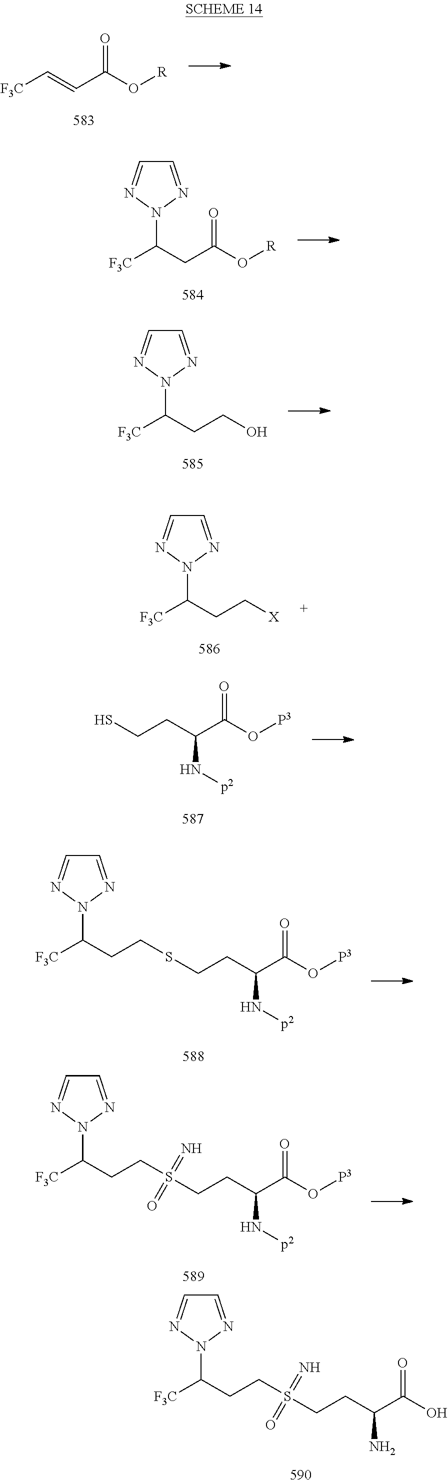

Alternative synthetic methods for variations of compounds 590 can be found in Scheme 14. Reaction of an appropriately substituted unsaturated ester, such as 583, with a nucleophile, such as triazole, may form the substituted ester product 584. Further reaction of 584 with a reducing agent, such as LAH, may form the primary alcohol 585, which can be further reacted with an activating agent, such as MsCl in the presence of an amine to form compounds such as 586. Compounds like 586 may be reacted with the bis-protected thiol 587 to form the dialkyl sulfide product 588. Compound 588 may be reacted with a variety of oxidizing reagents to provide the sulfoximine derivative 589 which may be further treated with reagents, such as TFA or HCl, to remove the amine protecting group and provide compounds 590.

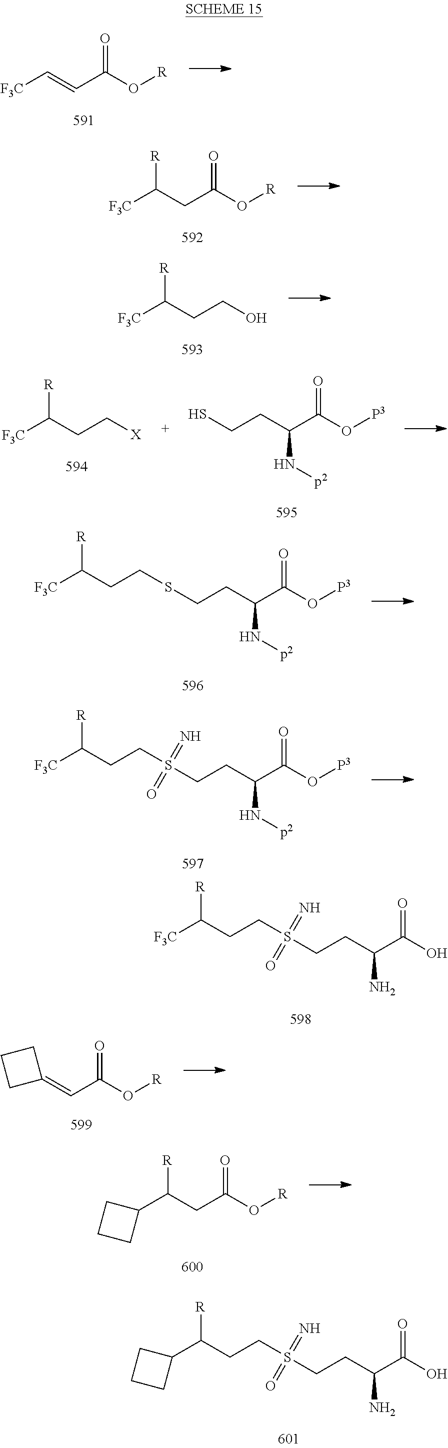

Additional synthetic methods for variations of compounds 598 can be found in Scheme 15. Reaction of an appropriately substituted unsaturated ester, such as 591, with a coupling partner, such as an aryl boronate in the presence of a transition metal catalyst, such as rhodium, may form the substituted ester product 592. Further reaction of 592 with a reducing agent, such as LAH, may form the primary alcohol 593, which can be further reacted with an activating agent, such as MsCl in the presence of an amine to form compounds such as 594 wherein X=mesylate. Compounds like 594 may be reacted with the bis-protected thiol 595 to form the dialkyl sulfide product 596. Compound 596 may be reacted with a variety of oxidizing reagents to provide the sulfoximine derivative 597 which may be further treated with reagents, such as NaOH to remove the acid protecting group (P³) TFA or HCl, to remove the amine protecting group (P²) and provide compounds 598.

A similar synthetic approach starting from the cyclobutyl substituted conjugated ester 599 may provide intermediates 600, either by reacting with a boron containing coupling partner or an alkyl or aryl halide under appropriate coupling conditions, such as Iridium/blue light catalysis. Further transformations, similar to the method already outlined for Scheme 15, may be utilized to provide additional compounds 601.

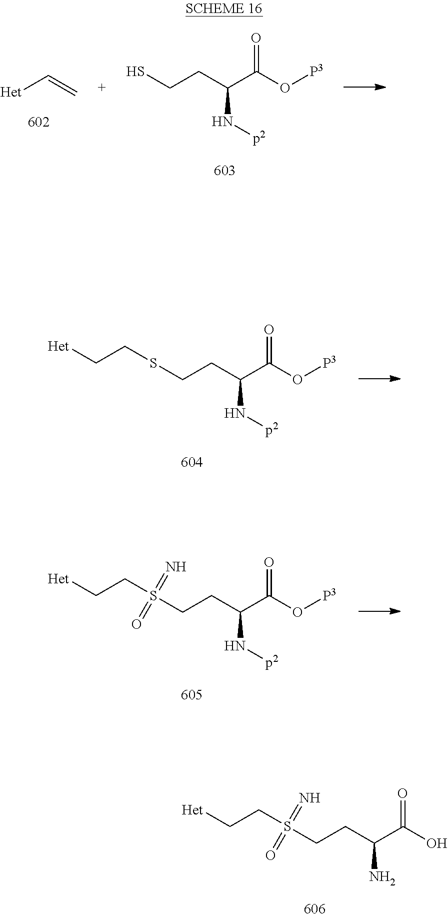

Scheme 16 shows how one skilled in the art may prepare additional compounds by reacting aryl or heteroaryl alkenes with the thiol 603, in an appropriate solvent, such as methanol, to form adducts such as 604. Compound 604 may be reacted with a variety of oxidizing reagents to provide the sulfoximine derivative 605 which may be further treated with reagents, such as NaOH to remove the acid protecting group (P³) TFA or HCl, to remove the amine protecting group (P²) and provide compounds 606.

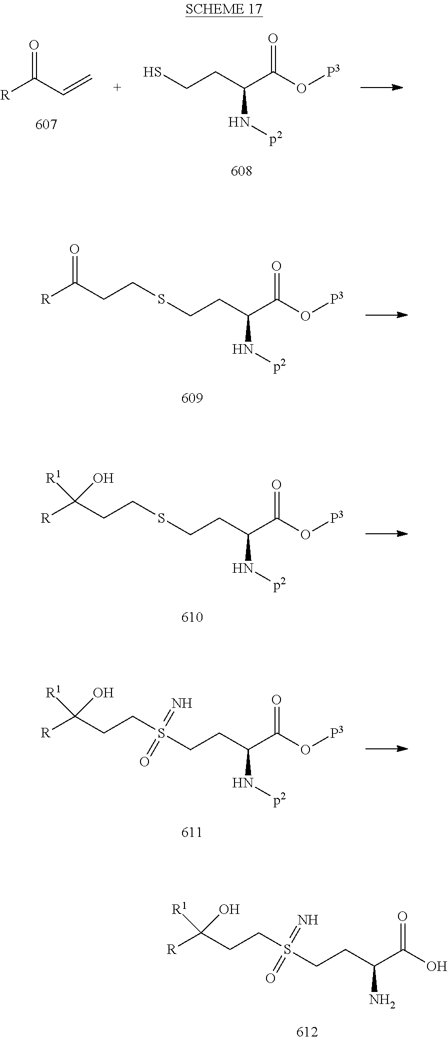

Another method for preparing compounds 612 is outlined in Scheme 17. Reacting compound 607 with compound 608 in an appropriate solvent in the presence of a base, such as TEA, may afford intermediate 609. Reaction of 609 with various alky or aryl Grignard or lithium species may provide tertiary alcohol derivative 610. Alternatively, 609 may be reacted with a trifluoromethylating reagent, such as Trimethylsilyl trifluoromethane in the presence of TBAF to provide analogs like 611 where R¹═CF₃. Compound 610 may be reacted with a variety of oxidizing reagents to provide the sulfoximine derivative 611 which may be further treated with reagents, such as NaOH to remove the acid protecting group (P³) TFA or HCl, to remove the amine protecting group (P²) and provide compounds 612.

In cases where R or R¹ is aryl or heteroaryl that may be substituted with one or more halogens or alternative functionality compatible with cross coupling reactions, further reaction with cross coupling reagents, such as substituted boronic acids, may further produce additional compounds 612. It should be appreciated that these types of reactions may be incorporated at various stages of the synthesis and may depend on cross reactivity or protecting group modifications.

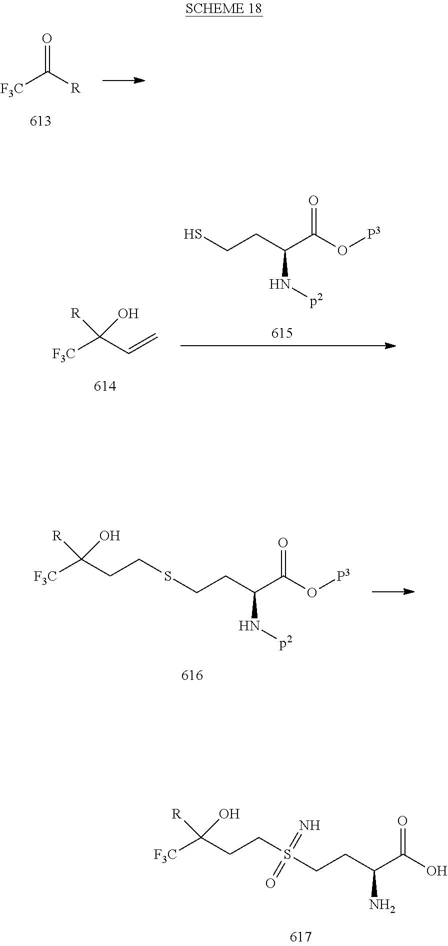

Additional analogs may be prepared starting from compound 613 by reacting with vinyl magnesium bromide in an appropriate solvent, such as THF, to provide 614. Reaction of 614 with 615 in the presence of AIBN may afford compounds such as 616. Compound 616 may be reacted with a variety of oxidizing reagents to produce the intermediate sulfoximine, which may be further treated with reagents, such as NaOH to remove the acid protecting group (P³) TFA or HCl, to remove the amine protecting group (P²) and provide compounds 617.

In cases where R is aryl or heteroaryl that may be substituted with halogens or alternative functionality compatible with cross coupling reactions, further reaction with cross coupling reagents, such as substituted boronic acids, may further produce additional compounds 617. It should be appreciated that these types of reactions may be incorporated at various stages of the synthesis and may depend on cross reactivity or protecting group modifications.

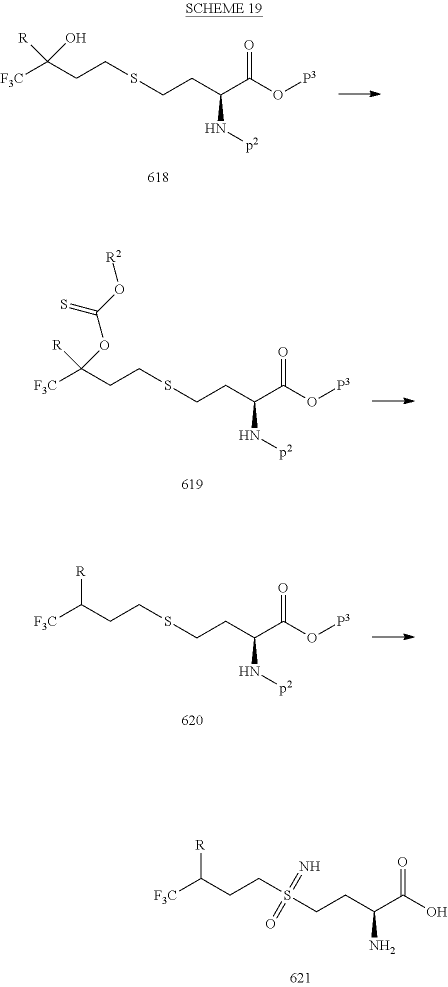

Additional analogs may be prepared starting from compound 618 by reacting the alcohol group with a thiocarbonate forming reagent, such as phenyl chlorothionoformate, to afford 619. Compound 619 may be further treated with a radical initiating reagent, such as AIBN, to provide the deoxygenated analog 620. Compound 620 may be reacted with a variety of oxidizing reagents to produce the intermediate sulfoximine, which may be further treated with reagents, such as NaOH to remove the acid protecting group (P³) TFA or HCl, to remove the amine protecting group (P²) and provide compounds 621. In cases where R is aryl or heteroaryl that may be substituted with halogens or alternative functionality compatible with cross coupling reactions, further reaction with cross coupling reagents, such as substituted boronic acids, may further produce additional compounds 621. It should be appreciated that these types of reactions may be incorporated at various stages of the synthesis and may depend on cross reactivity or protecting group modifications.

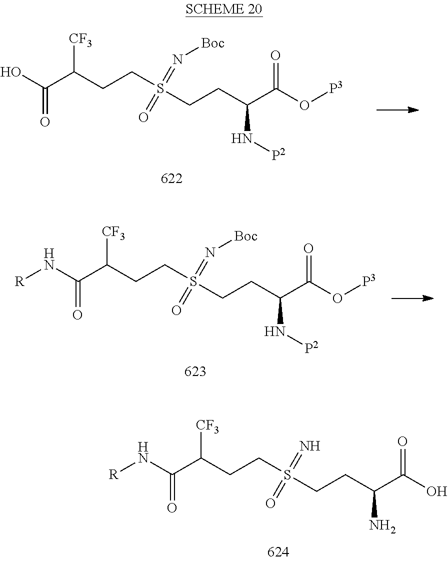

Additional substituted analogs may be prepared by the route outlined in Scheme 20. Starting with the carboxylic acid derivative 622, reacting with an amine in the presence of an amide bond forming reagent, such as HATU, may provide 623. Removal of the protecting groups with TFA and/or strong base can provide the amide derived analogs 624.

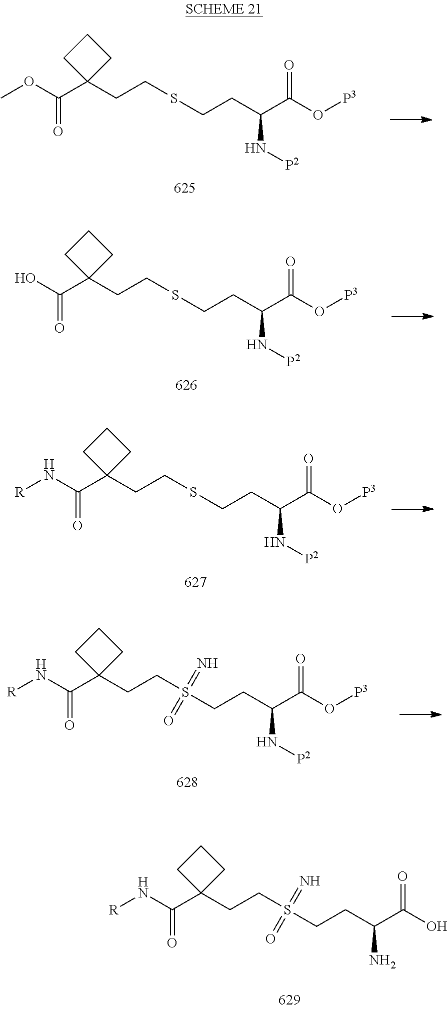

Additional amide substituted analogs may be prepared by the route outlined in Scheme 21. The cyclobutyl ester derivative 625 can be treated with a hydrolyzing reagent or enzyme, such as pig liver esterase, to afford the acid 626. Acid 626 may be reacted with an amine in the presence of an amide bond forming reagent, such as HATU, to provide 627. Compound 627 may be reacted with a variety of oxidizing reagents to produce the intermediate sulfoximine 628, which may be further treated with reagents, such as NaOH to remove the acid protecting group (P³) TFA or HCl, to remove the amine protecting group (P²) and provide compounds 629.

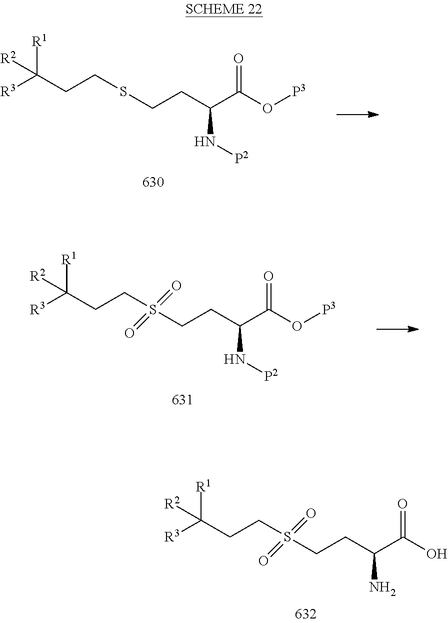

Sulfone derived analogs may be prepared according to the method outlined in Scheme 22. Starting from readily available intermediates, such as the dialkyl sulfide 630, treatment with an oxidizing agent, such as mCPBA, can for the sulfone intermediate 631. Treatment of 631 with acid-based deprotecting reagents, such as HCl in dioxane, can provide compounds 632. It should also be appreciated that the R¹, R², and R³ groups may be functionalized and derivatized at various stages of the process. For example, if R¹ is a bromophenyl group, one may react with an aryl-boronic acid in the presence of a catalyst, such as palladium, to provide a bi-aryl substituted analog.

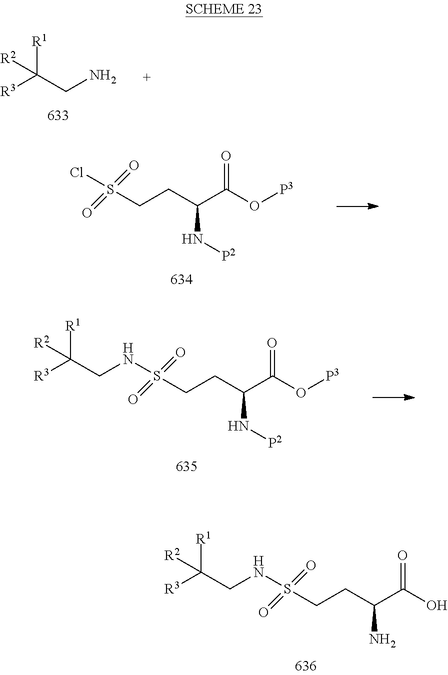

Sulfonamide derived analogs may be prepared according to the method outlined in Scheme 23. Starting from readily available intermediates, such as a substituted amine 633, reaction with a sulfonyl chloride, such as 634 in the presence of a base may provide compounds such as 635. Treatment of 635 with acid-based deprotecting reagents, such as HCl in dioxane, can provide compounds 636. It should also be appreciated that the R¹, R², and R³ groups may be functionalized and derivatized at various stages of the process. For example, if R¹ is a bromophenyl group, one may react with an aryl-boronic acid in the presence of a catalyst, such as palladium, to provide a bi-aryl substituted analog.

### Pharmaceutical Compositions

Provided herein are pharmaceutical compositions, wherein the pharmaceutical compositions comprise an agent selected from Table 1 or a combination of agents selected from Table 1 and/or Table 2 and/or Table 3 and/or Table 4; and a pharmaceutically acceptable excipient. In some embodiments, the pharmaceutical composition further comprises a cell death inducing agent. In some embodiments, the pharmaceutical composition further comprises a chemotherapeutic agent. In some embodiments, pharmaceutical compositions provided herein are in a suspension, optionally a homogeneous suspension. In some embodiments, pharmaceutical compositions provided herein are in an emulsion form. In some embodiments, pharmaceutical compositions provided herein comprise a salt form of any one of the agents provided herein. In some embodiments, the salt is a methanesulfonate salt.

Also provided herein is a pharmaceutical composition comprising a ferroptosis-inducing agent or an iron-dependent cell death agent provided herein. In some embodiments, agents provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Exemplary carriers and excipients can include dextrose, sodium chloride, sucrose, lactose, cellulose, xylitol, sorbitol, malitol, gelatin, polymers, polyethylene glycol (PEG), polyvinylpyrrolidone (PVP), and any combination thereof. In some embodiments, an excipient such as dextrose or sodium chloride can be at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier or diluent such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Ferroptosis-inducing agents provided herein can be formulated as a rectal composition, for example, suppositories or retention enemas, for example, containing conventional suppository bases, for example, cocoa butter or other glycerides, or gel forming agents, such as carbomers.

Pharmaceutical compositions also can be administered by controlled release formulations and/or delivery devices (see, e.g., in U.S. Pat. No. 5,733,566).

Various delivery vehicles are known and can be used to administer ferroptosis-inducing agents provided herein, such as but not limited to, encapsulation in liposomes, microparticles, microcapsules, nanoparticles, vectors, and recombinant cells. Liposomes and/or nanoparticles also can be employed with administration of compositions herein. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MHLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles can also be used as a delivery vehicle.

Nanoparticle carriers that specifically target a tissue provided herein may also be used as a pharmaceutically acceptable carrier. In some embodiments, the nanoparticle is a gold nanoparticle, a platinum nanoparticle, an iron-oxide nanoparticle, a lipid nanoparticle, a selenium nanoparticle, a tumor-targeting glycol chitosan nanoparticle (CNP), a cathepsin B sensitive nanoparticle, a hyaluronic acid nanoparticle, a paramagnetic nanoparticle, or a polymeric nanoparticle.

Suitable pharmaceutical formulations of ferroptosis-inducing agents for transdermal application include an effective amount of an agent with a carrier. Carriers include absorbable pharmacologically acceptable solvents to assist passage through the skin of the subject. For example, transdermal devices are in the form of a bandage or patch comprising a backing member, a reservoir containing the compound optionally with carriers, optionally a rate controlling barrier to deliver the compound to the skin of the host at a controlled and predetermined rate over a prolonged period of time, and a means to secure the device to the skin. Matrix transdermal formulations may also be used. Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In certain embodiments, ferroptosis-inducing agents provided herein are formulated as a depot composition. Such long-acting formulations can be administered by implantation (for example, subcutaneously or intramuscularly) or by intramuscular injection. The ferroptosis-inducing agents can be formulated with suitable polymeric or hydrophobic materials (for example as an emulsion in an acceptable oil), ion exchange resins, biodegradable polymers, or as sparingly soluble derivatives, for example, as a sparingly soluble salt.

In some embodiments, one or more agent provided herein is formulated as a pharmaceutical food composition (also referred to as a medical food). The food composition can be for consumption by a mammal, for example by a human or a non-human mammal. Agents provided herein can be formulated as a dietary supplement or a medical food. In some embodiments, agents provided herein are administered with a food ingredient. A food ingredient is any product, composition, or a component of a food known to have or disclosed as having a nutritional effect. Food can include various meats (e.g., beef, pork, poultry, fish, etc.), dairy products (e.g., milk, cheese, eggs), fruits, vegetables, cereals, breads, etc., and components thereof. Food can be fresh or preserved, e.g., by canning, dehydration, freezing, or smoking. Food can be provided in raw, unprepared and/or natural states or in cooked, prepared, and/or combined states. In some embodiments, the food ingredient is selected from the group consisting of: fat, carbohydrates, protein, fiber, nutritional balancing agent, and mixtures thereof. In some embodiments, the pharmaceutical food composition provided herein further comprises one or more of a protein or an amino acid. In some embodiments of any of the aspects, the pharmaceutical food composition further comprises adenine, one or more vitamins (e.g., vitamin E), potassium, fatty acids, and/or calcium carbonate.

### Methods of Administering an Agent

Provided herein can be methods for administering a therapeutic regime to a subject having a disease or disorder (e.g., cancer, an autoimmune disease, or fibrosis). In some embodiments, the administering is sustained administration of a therapeutically effective amount of a ferroptosis-inducing agent. In some embodiments, the sustained administration of the ferroptosis-inducing agent comprises providing to a tissue the ferroptosis-inducing agent in an amount sufficient to achieve a distribution of at least about 10 ng/mm² within said tissue for a period of at least 4 hours, thereby inducing ferroptosis in the tissue. In some embodiments, the sustained administration further forms a gradient of a sub-therapeutic amount of the ferroptosis-inducing agent adjacent to an administration site within the tissue. In some embodiments, sustained administration of the ferroptosis-inducing agent comprises additional administration steps. In some embodiments, the ferroptosis-inducing agent is administered more than once. In some embodiments, the administering is via a system provided herein. In some embodiments, the administering local administration within a tissue. In some embodiments, the tissue is contacted in vivo with an effective amount of an iron-dependent cell death agent for a duration of time of at least 4 hours. In some embodiments, the administering comprises contacting a mammalian tissue with a priming agent and contacting the mammalian tissue with an effective amount of a ferroptosis-inducing agent provided herein, wherein the ferroptosis-inducing agent induces targeted cell death in the mammalian tissue in vivo. In some embodiments, the administering is local administration or systemic administration. In some embodiments, the administering or contacting step is via intratumoral injection, oral administration, transdermal injection, inhalation, nasal administration, topical administration, vaginal administration, ophthalmic administration, intracerebral administration, rectal administration.

In some instances, an agent or combination of agents provided herein are administered as a unit dosage form. Many agents can be administered orally as liquids, capsules, tablets, or chewable tablets. Because the oral route is the most convenient and usually the safest and least expensive, it is the one most often used. However, it has limitations because of the way a drug typically moves through the digestive tract. For agents administered orally, absorption may begin in the mouth and stomach. However, most agents are usually absorbed from the small intestine. The drug passes through the intestinal wall and travels to the liver before being transported via the bloodstream to its target site. The intestinal wall and liver chemically alter (metabolize) many agents, decreasing the amount of drug reaching the bloodstream. Consequently, these agents are often given in smaller doses when injected intravenously to produce the same effect.

In some embodiments, an agent provided herein is formulated for oral administration. In some embodiments, an agent provided herein is formulated for administration/for use in administration via a subcutaneous, intradermal, intramuscular, inhalation, intravenous, intraperitoneal, intracranial, intrathecal, intratumoral, or oral route. For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Agents are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. In some cases, agents or therapeutic regimes are administered as infusions. An infusion can take place over a period of time. For example, an infusion can be an administration of an agent or therapeutic regime over a period of about 5 minutes to about 5 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, or up to about 5 hours.

In some embodiments, intravenous administration is used to deliver a precise dose quickly and in a well-controlled manner throughout the body. It is also used for irritating solutions, which would cause pain and damage tissues if given by subcutaneous or intramuscular injection. An intravenous injection can be more difficult to administer than a subcutaneous or intramuscular injection because inserting a needle or catheter into a vein may be difficult, especially if the person is obese. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some agents must be given by continuous infusion to keep their effect constant. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

For administration by inhalation, the ferroptosis-inducing agent provided herein can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch. Agents administered by inhalation through the mouth can be atomized into smaller droplets than those administered by the nasal route, so that the agents can pass through the windpipe (trachea) and into the lungs. How deeply into the lungs the agents go depends on the size of the droplets. Smaller droplets go deeper, which increases the amount of drug absorbed. Inside the lungs, they are absorbed into the bloodstream.

Agents applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In some cases, a treatment regime may be dosed according to a body weight of a subject. In subjects who are determined obese (BMI>35) a practical weight may need to be utilized. BMI is calculated by: BMI=weight (kg)/[height (m)]².

In some cases, a therapeutic regime can be administered along with a carrier or excipient. Ferroptosis-inducing agents provided herein can be administered with one or more of a second agent, sequentially, or concurrently, either by the same route or by different routes of administration. When administered sequentially, the time between administrations is selected to benefit, among others, the therapeutic efficacy and/or safety of the combination treatment. In certain embodiments, the agents provided herein can be administered first followed by a second agent, or alternatively, the second agent is administered first followed by the agents of the present disclosure (e.g., ferroptosis-inducing agents of Table 1). By way of example and not limitation, the time between administrations is about 1 hr, about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In some embodiments, the time between administrations is about 1 week, 2 weeks, 3 weeks, or 4 weeks or more. In some embodiments, the time between administrations is about 1 month or 2 months or more.

In some embodiments, ferroptosis-inducing agents provided herein contact the mammalian tissue for at least about 4 hours, at least about 6 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 36 hours, at least about 48 hours, up to 72 hours. In some embodiments, ferroptosis-inducing agents provided herein contact the mammalian tissue for about 4 hours. In some embodiments, ferroptosis-inducing agents provided herein contact the mammalian tissue for about 6 hours. In some embodiments, ferroptosis-inducing agents provided herein contact the mammalian tissue for about 10 hours. In some embodiments, ferroptosis-inducing agents provided herein contact the mammalian tissue for about 12 hours. In some embodiments, ferroptosis-inducing agents provided herein contact the mammalian tissue for about 24 hours. In some embodiments, ferroptosis-inducing agents provided herein contact the mammalian tissue for about 48 hours. In some embodiments, ferroptosis-inducing agents provided herein contact the mammalian tissue for about 72 hours.

When administered concurrently, the agent can be administered separately, at the same time as the second agent, by the same or different routes, or administered in a single pharmaceutical composition by the same route. In certain embodiments, the amount and frequency of administration of the second agent can used standard dosages and standard administration frequencies used for the particular compound.

### Dosing and Tissue Distribution

The methods provided herein comprise administering to a subject an agent or pharmaceutical composition provided herein in an amount effective to induce ferroptosis in a tissue in vivo. Agents and pharmaceutical compositions for administering to a subject in need thereof may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model may also be used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

A typical human dose of an agent provided herein (e.g., a ferroptosis-inducing agent) may be from about 10 μg/kg body weight/day to 10,000 mg/kg/day. In some embodiments, the dose of an agent provided herein is from about 0.1 mg/kg to about 1000 mg/kg, from 1 mg/kg to 1000 mg/kg, 1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg. In certain embodiments, the suitable dosages of the agent can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 2,000 mg/kg, 3,000 mg/kg, 4,000 mg/kg, 5,000 mg/kg, 6,000 mg/kg, 7,000/mg/kg, 8,000 mg/kg, 9,000 mg/kg, up to 9,600 mg/kg. In some embodiments, the dose of an agent provided herein is from about 100 mg/kg/day to about 6,400 mg/kg/day four times per day. In some embodiments, the dose of an agent provided herein is from about 50 mg/kg/day to about 25 mg/kg/day. In some embodiments, the dose of an agent provided herein is from about 400 mg/kg/day to about 800 mg/kg/day. In certain embodiments, the dose of the agent can be administered once per day or divided into subdoses and administered in multiple doses, e.g., twice, three times, or four times per day.

In some embodiments, agents provided herein are administered in an amount of at least about 10 nanograms (ng) or more, about 20 ng or more, about 30 ng or more, about 40 ng or more, about 50 ng or more, about 60 ng or more, about 70 ng or more, about 80 ng or more, about 90 ng or more, up to 100 ng. In some embodiments, the agent is administered in an amount of at least about 1 microgram (μg) or more, about 5 μg or more, about 10 μg or more, about 20 μg or more, about 30 μg or more, about 40 μg or more, about 50 μg or more, about 60 μg or more, about 70 μg or more, about 80 μg or more, about 90 μg or more, up to 100 μg.

In some embodiments, agents provided herein are administered at a concentration of at least about 0.1 micromolar (μM) or more, about 1 μM or more, about 2 μM or more, about 3 μM or more, about 4 μM or more, about 5 μM or more, about 6 μM or more, about 7 μM or more, about 8 μM or more, about 9 μM or more, about 10 μM or more, about 15 μM or more, about 20 μM or more, about 25 μM or more, about 30 μM or more, about 35 μM or more, about 40 μM or more, about 45 μM or more, about 50 μM or more, about 55 μM or more, about 60 μM or more, about 65 μM or more, about 70 μM or more, about 75 μM or more, about 80 μM or more, about 85 μM or more, about 90 μM or more, about 95 μM or more, about 100 μM or more, about 110 μM or more, about 120 μM or more, about 130 μM or more, about 140 μM or more, about 150 μM or more, about 160 μM or more, about 170 μM or more, about 180 μM or more, about 190 μM or more, about 200 μM or more, about 300 μM or more, about 400 μM or more, about 500 μM or more, up to 1 mM. In some embodiments, agents provided herein are administered at a concentration of at least about 0.1 μM up to about 500 μM. In some embodiments, agents provided herein are administered at a concentration of at least about 1 μM up to 500 μM. In some embodiments, agents provided herein are administered at a concentration of at least about 0.1 μM up to 10 μM. In some embodiments, agents provided herein are administered at a concentration of at least about 1 μM up to 10 μM.

In some embodiments, ferroptosis-inducing agents provided herein are administered intravenously. In some embodiments, ferroptosis-inducing agents provided herein are administered intravenously at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg. In some embodiments, ferroptosis-inducing agents provided herein are administered intravenously at a concentration of about 25 mg/kg once per day. In some embodiments, ferroptosis-inducing agents provided herein are administered intravenously at a concentration of about 25 mg/kg twice per day. In some embodiments, ferroptosis-inducing agents provided herein are administered intravenously at a concentration of about 450 mg/kg/day. In some embodiments, ferroptosis-inducing agents provided herein are administered intravenously at a concentration of about 650 mg/kg/day. In some embodiments, ferroptosis-inducing agents provided herein are administered intravenously at a concentration of about 650 mg/kg/day for 3 continuous days. In some embodiments, ferroptosis-inducing agents provided herein are administered intravenously at a concentration of about 1300 mg/kg/day. In some embodiments, ferroptosis-inducing agents provided herein are administered intravenously at a concentration of about 2400 mg/kg/day.

In some embodiments, ferroptosis-inducing agents provided herein are administered orally. In some embodiments, ferroptosis-inducing agents provided herein are administered orally at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg. In some embodiments, ferroptosis-inducing agents provided herein are administered orally at a concentration of about 25 mg/kg once per day. In some embodiments, ferroptosis-inducing agents provided herein are administered orally at a concentration of about 25 mg/kg twice per day. In some embodiments, ferroptosis-inducing agents provided herein are administered orally at a concentration of about 1300 mg/kg/day. In some embodiments, ferroptosis-inducing agents provided herein are administered orally at a concentration of about 2400 mg/kg/day.

The methods provided herein can be characterized by or further comprise measuring the distribution of an agent in a target tissue. Distribution of an agent provided herein can be determined by the amount or concentration of the agent within a square millimeter (mm²) or cubic millimeter (mm³) of tissue. For example, for local administration of an agent to a tumor, the tissue may be from about 6 to 7 mm in diameter, 36 to 42 mm², or 216 to 294 mm³. The data obtained from animal studies may be used in formulating a range of drug distribution in a mammalian tissue. Methods of determining tissue distribution of a drug or agent include, for example, mass spectrometry, chromatography, imaging techniques, and immunoassays. The distribution of an agent provided herein can be determined using a system provided herein.

In some embodiments, the tissue is administered a therapeutic amount of a ferroptosis-inducing agent, compound, enantiomer of any of the foregoing, diastereomer of any of the foregoing, pharmaceutically acceptable salt of any of the foregoing, or deuterated derivative of any of the foregoing, wherein administration of comprises providing to a tissue the ferroptosis-inducing agent in an amount sufficient to achieve a desired drug distribution. In some embodiments, the agent, compound, enantiomer of any of the foregoing, diastereomer of any of the foregoing, pharmaceutically acceptable salt of any of the foregoing, or deuterated derivative of any of the foregoing provided herein achieve a distribution within a tissue of at least about 1 ng/mm² or more, about 5 ng/mm² or more, about 10 ng/mm² or more, about 15 ng/mm² or more, about 20 ng/mm² or more, about 25 ng/mm² or more, about 30 ng/mm² or more, about 35 ng/mm² or more, about 40 ng/mm² or more, about 45 ng/mm² or more, about 50 ng/mm² or more, about 55 ng/mm² or more, about 60 ng/mm² or more, about 65 ng/mm² or more, about 70 ng/mm² or more, about 75 ng/mm² or more, about 80 ng/mm² or more, about 85 ng/mm² or more, about 90 ng/mm² or more, about 95 ng/mm² or more, about 100 ng/mm² or more, about 110 ng/mm² or more, about 120 ng/mm² or more, about 130 ng/mm² or more, about 140 ng/mm² or more, about 150 ng/mm² or more, about 160 ng/mm² or more, about 170 ng/mm² or more, about 180 ng/mm² or more, about 190 ng/mm² or more, about 200 ng/mm² or more, about 300 ng/mm² or more, about 400 ng/mm² or more, up to 500 ng/mm². In some embodiments, the agent, compound, enantiomer of any of the foregoing, diastereomer of any of the foregoing, pharmaceutically acceptable salt of any of the foregoing, or deuterated derivative of any of the foregoing, provided herein achieve a distribution within a tissue of at least about 1 ng/mm³ or more, about 5 ng/mm³ or more, about 10 ng/mm³ or more, about 15 ng/mm³ or more, about 20 ng/mm³ or more, about 25 ng/mm³ or more, about 30 ng/mm³ or more, about 35 ng/mm³ or more, about 40 ng/mm³ or more, about 45 ng/mm³ or more, about 50 ng/mm³ or more, about 55 ng/mm³ or more, about 60 ng/mm³ or more, about 65 ng/mm³ or more, about 70 ng/mm³ or more, about 75 ng/mm³ or more, about 80 ng/mm³ or more, about 85 ng/mm³ or more, about 90 ng/mm³ or more, about 95 ng/mm³ or more, about 100 ng/mm³ or more, about 110 ng/mm³ or more, about 120 ng/mm³ or more, about 130 ng/mm³ or more, about 140 ng/mm³ or more, about 150 ng/mm³ or more, about 160 ng/mm³ or more, about 170 ng/mm³ or more, about 180 ng/mm³ or more, about 190 ng/mm³ or more, about 200 ng/mm³ or more, about 300 ng/mm³ or more, about 400 ng/mm³ or more, up to 500 ng/mm³.

In some embodiments, the agent, compound, enantiomer of any of the foregoing, diastereomer of any of the foregoing, pharmaceutically acceptable salt of any of the foregoing, or deuterated derivative of any of the foregoing, provided herein are administered at least about once per day, twice per day, three times per day, four times per day, or five times per day. In some embodiments of any of the aspects, ferroptosis-inducing agents are administered at least about every week, at least about every 2 weeks, or at least about every 3 weeks. The amount of drug administered depends on the size of the tissue, the type of disease being treated, and the type of administration (e.g., local administration to a tissue in vivo using a system provided herein). Effective doses will vary, depending on the types of diseases treated, route of administration, excipient usage, and the possibility of co-usage with other therapeutic treatments.

### Efficacy

Therapeutic efficacy of an agent and/or pharmaceutical composition provided herein may be determined by evaluating and comparing patient symptoms and quality of life pre- and post-administration. Such methods apply irrespective of the mode of administration. In some embodiments, pre-administration refers to evaluating patient symptoms and quality of life prior to onset of therapy and post-administration refers to evaluating patient symptoms and quality of life at least 1, 2, 3, 4, 5, 6, 7, 8, 9, 10, 11, 12, 13, 14, 15, 16, 17, 18, 19, 20 weeks after onset of therapy. In some embodiments, pre-administration refers to evaluating patient symptoms and quality of life prior to onset of therapy and post-administration refers to evaluating patient symptoms and quality of life of up to 52 weeks after onset of therapy. In a particular embodiment, the post-administration evaluating is performed about 2-8, 2-6, 4-6, or 4 weeks after onset of therapy. In a particular embodiment, patient symptoms (e.g., symptoms related to cancer, fibrosis, or autoimmune disease) and quality of life pre- and post-administration are evaluated clinically and by questionnaire assessment.

The agents and methods provided herein can be used to reduce cancer cell proliferation or survival in vivo or in vitro. Methods of evaluating tumor progression or cell proliferation are known in the art. In some embodiments, overall response is assessed from time-point response assessments (based on tumor burden) as follows:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

In some embodiments, an in vitro cell proliferation assay is used to assess the efficacy of a one or more ferroptosis-inducing agents provided herein. The compositions and methods provided herein result in a reduction in the proliferation or survival of a plurality of cells. For example, after treatment with one or more of the agents provided herein, cell proliferation or survival is reduced by 5% or greater (e.g., 10%, 20%, 30%, 40%, 50%, 60%, 70%, 80%, 90% or greater) relative to cell proliferation or survival prior to treatment.

In some embodiments, animal models are used to assess the efficacy of a one or more ferroptosis-inducing agents provided herein in vivo. The ferroptosis-inducing agents and methods provided herein can result in a reduction in size or volume of a hyperproliferating tissue (e.g., a tumor). For example, after treatment, tissue size is reduced by 5% or greater (e.g., 10%, 20%, 30%, 40%, 50%, 60%, 70%, 80%, 90% or greater) relative to its size prior to treatment. Size of a tissue (e.g., a tumor) may be measured by any reproducible means of measurement. The size of a tissue may be measured as a diameter of the tumor or by any reproducible means of measurement. Ferroptosis inhibitors (e.g., an agent in Table 2 or Table 3) may be used to determine the efficacy of a particular test agent (also referred to herein as an active agent) for inducing ferroptosis in a tissue. For example, the combination of a ferroptosis inducer paired with a ferroptosis inhibitor (e.g., liproxstatin-1) can be used to determine whether the test agent targets a protein or nucleic acid involved in the ferroptosis pathway (see FIG. 1). Further provided herein is a method of rescuing a cell or plurality of cells from cell death and/or ferroptosis in vivo, the method comprising: administering to a subject a ferroptosis inhibitor. In some embodiments, the method further comprises administering a ferroptosis-inducing agent. Further provided herein is a method of screening a plurality of cells in a tissue for ferroptosis-sensitivity, the method comprising: contacting the tissue with a ferroptosis-inducing agent and a ferroptosis inhibitor; and measuring one or more parameters indicative of ferroptosis. In some embodiments, the ferroptosis-inducing agent is an agent in Table 1 or a test agent. In some embodiments, the ferroptosis inhibitor is any agent listed in Table 2 or Table 3. In some embodiments, the ferroptosis inhibitor is any agent listed in Table 4. In some embodiments, the ferroptosis inducing agent is any agent listed in Table 2 or Table 3. In some embodiments, the ferroptosis inhibitor is liproxstatin-1. In some embodiments, the one or more parameters indicative of ferroptosis are PUFA concentration, PI index, modulation of mesenchymal cell state marker expression, or modulation of iron or selenium concentration. The screening method provided herein can be readily scaled for high throughput analyses, that permit evaluation or prediction of the ferroptosis-inducing activity of test agents. Similarly, the screening method can be performed in animal models as discussed above in the presence and absence of a ferroptosis inhibitor.

Treating a disease or disorder (e.g., cancer) can further result in a decrease in number of hyperproliferative tissues (e.g., tumors). For example, after treatment, hyperproliferative tissue or tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×). In some embodiments, methods and ferroptosis-inducing agents provided herein increase the number or activity of leukocytes in a tumor microenvironment. In some embodiments, the leukocytes specifically target cancer cells with a high PUFA concentration as compared with normal cells.

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

Treating a disease or disorder (e.g., cancer) can result in an increase in average survival time of a population of subjects treated according to the present disclosure in comparison to a population of untreated subjects. For example, the average survival time is increased by more than 30 days (more than 60 days, 90 days, 120 days or longer). An increase in average survival time of a population may be measured by any reproducible means. An increase in average survival time of a population may be measured, for example, by calculating for a population the average length of survival following initiation of treatment with the compound of the disclosure. An increase in average survival time of a population may also be measured, for example, by calculating for a population the average length of survival following completion of a first round of treatment with the compound of the disclosure.

Treating a disease or disorder (e.g., cancer) can also result in a decrease in the mortality rate of a population of treated subjects in comparison to an untreated population. For example, the mortality rate is decreased by more than 2% (e.g., more than 5%, 10%, 25%, or greater). A decrease in the mortality rate of a population of treated subjects may be measured by any reproducible means, for example, by calculating for a population the average number of disease-related deaths per unit time following initiation of treatment with the compound of the disclosure. A decrease in the mortality rate of a population may also be measured, for example, by calculating for a population the average number of disease-related deaths per unit time following completion of a first round of treatment with a ferroptosis-inducing agent.

Treating a disease or disorder can also result in a decrease in at least one symptom associated with the disease, disorder, or condition. In some embodiments, the methods provided herein reduce at least one symptom of a disease or disorder by at least 10%, 20%, 30%, 40%, 50%, 70%, 80%, 90% or greater relative to number prior to treatment. In some embodiments, following contact with a mammalian tissue or administration of a ferroptosis-inducing agent, cell death can be detected at a time point at or after contacting the mammalian tissue with the ferroptosis-inducing agent. In some embodiments, the methods provided herein increase cell death by at least 5%, 10%, 20%, 30%, 40%, 50%, 60%, 70%, 80%, 90% or greater relative to number prior to treatment.

### Therapeutic Applications

Provided herein are methods of treating a disease or a disorder in a subject. In some embodiments, the subject has, is suspected of having, or is at risk of developing a hyperproliferative disease or condition. In some embodiments, methods provided herein further comprise a step of obtaining a biopsy of the tissue for histological analysis. In some embodiments, the tissue comprises a histological abnormality, wherein the histological abnormality is hyperplasia or fibrosis.

In some embodiments, the subject has, is suspected of having, or is at risk of developing a disease or condition associated with abnormal angiogenesis or vascularization. Diseases or conditions associated with abnormal angiogenesis or vascularization can include but are not limited to: ocular neovascularization, macular degeneration, retinopathy, sarcomas, polycystic kidney disease, benign hyperplasias, leiomyomas, adenomas, lipomas, hemangiomas, fibromas, vascular occlusion, restenosis, atherosclerosis, pre-neoplastic lesions, carcinoma in situ, and cancer. In some embodiments, the subject has, is suspected of having, or is at risk of developing an autoimmune disease. Non-limiting examples of relevant autoimmune diseases include: rheumatoid arthritis, inflammatory bowel disease, osteoarthritis, oral hairy leukoplakia, and psoriasis. In some embodiments, the subject has, is suspected of having, or is at risk of developing fibrosis. Non-limiting examples of diseases and conditions associated with fibrosis include: keloid scars, hypertrophic scars, systemic sclerosis, pulmonary arterial hypertension, cardiac fibrosis, hypertrophic cardiomyopathy valvular disease, myelofibrosis, myelodysplastic syndrome, chronic myelogenous leukemia, portal hypertension, hepatocellular carcinoma, retroperitoneal fibrosis, intestinal fibrosis, enteropathies, subretinal fibrosis, epiretinal fibrosis, cystic fibrosis, emphysema, pancreatic fibrosis, chronic pancreatitis, duct obstruction, arthrofibrosis, renal fibrosis, nephrogenic systemic fibrosis, renal anemia, chronic kidney disease, Dupuytren's disease, Ledderhose disease (plantar fibromatosis), primary biliary cholangitis (PBC), non-alcoholic steatohepatitis (NASH), scleroderma, diabetic neuropathy, hypertensive nephrosclerosis, allograft nephropathy, cirrhosis, and pulmonary fibrosis.

In some embodiments, the subject has, is suspected of having, or is at risk of developing cancer. In some embodiments, the subject has a benign tumor. In some embodiments, the subject has a pre-cancerous lesion. In some embodiments, the subject has a basal cell carcinoma (BCC) or a squamous cell carcinoma (SCC). In some embodiments, the subject has a metastatic tumor. In some embodiments, the cancer is a solid cancer or a blood cancer. In some embodiments, the blood cancer is a leukemia or a lymphoma. In some embodiments, the subject has a solid tumor. In some embodiments, the solid tumor is a carcinoma, a melanoma, or a sarcoma. In some embodiments, the melanoma is a dedifferentiated melanoma or amelanotic melanoma. In some embodiments, the subject has a melanoma with a B-Raf proto-oncogene, serine/threonine kinase (BRAF) mutation. In some embodiments the subject has a sarcoma with a Kirsten rat sarcoma (KRAS) mutation. In some embodiments, the sarcoma is a soft tissue sarcoma. In some embodiments, the sarcoma is leiomyosarcoma. In some embodiments, the carcinoma is a colon adenocarcinoma. In some embodiments, the carcinoma is a liver carcinoma. In some embodiments, the carcinoma is renal carcinoma. In some embodiments, the carcinoma is clear cell renal carcinoma. In some embodiments, the carcinoma is non-clear cell renal carcinoma.

Non-limiting examples of cancer that can be treated with an agent provided herein include: acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CIVIL, T cell CML), and chronic lymphocytic leukemia (CLL) (e.g., B cell CLL, T cell CLL)); lymphoma such as Hodgkin lymphoma (HL) (e.g., B cell HL, T cell HL) and non Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma, clear cell renal carcinoma, non-clear cell renal carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), angiogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); colorectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Provided herein are methods of administering a ferroptosis-inducing agent to a tissue, wherein the tissue comprises different cell types. In some embodiments, the tissue comprises a heterogeneous population of cells, wherein the heterogeneous population of cells comprises at least one of precancerous cells and non-cancerous cells. In some embodiments, the tissue comprises a heterogeneous population of cells, wherein the heterogeneous population of cells comprises a population of immune cells.

Provided herein is a method of inducing immune cell recruitment to a tumor, the method comprising: administering to a subject a ferroptosis-inducing agent provided herein by any of the methods provided herein. In some embodiments, the administering is sustained administration for at least about 10 hours, thereby recruiting immune cells to the tumor site. In some embodiments, the immune cells are leukocytes. In some embodiments, following contact with a mammalian tissue or administration of a ferroptosis-inducing agent, immune cell recruitment can be detected at a time point at or after contacting the mammalian tissue with the ferroptosis-inducing agent. In some embodiments, the administering reduces the size of the tumor and/or increases the number of leukocytes within the tumor.

### Systems

Provided herein are systems for the delivery of a ferroptosis-inducing agent or an iron-dependent cell death inducing agent provided herein. Further provided herein are systems for inducing in vivo ferroptosis, the systems comprising: an implantable microdevice configured for localized administration to a tissue comprising: (a) a cylindrical support structure having at least one microwell on a surface of or formed within the support structure; (b) a microdose of a ferroptosis-inducing agent in the at least one microwell; and (c) a compound release mechanism for sustained administration for controlling a release of the ferroptosis-inducing agent from the microwell, wherein the microdose of the ferroptosis-inducing agent forms a gradient of a sub-therapeutic dose of the ferroptosis-inducing agent an administration site within the tissue for a duration of time of at least 4 hours, wherein the microdevice is configured to permit implantation into the tissue using a catheter, cannula or biopsy needle, and wherein the microdevice is further configured to release the ferroptosis-inducing agent from the at least one microwell to the administration site within the apoptosis-resistant tissue adjacent to the at least one microwell.

Further provided herein are systems for identifying ferroptosis induction in an animal model comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) a micro-dose of the one or more active agents in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, and identifying one or more active agents induces ferroptosis in the tissue.

Further provided herein are systems for screening for ferroptosis-induced cell death in vivo, the systems comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) at least one microwell containing one or more ferroptosis inhibitors; (ii) a micro-dose of the one or more active agents; and/or one or more ferroptosis inhibitors in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, wherein the system measures an outcome of ferroptosis induction in the animal model after administration of the one or more active agents into the tissue relative to administration of the one or more active agents and one or more ferroptosis inhibitors, and identifying one or more active agents induces ferroptosis in the tissue.

The systems provided herein generally include multiple microwells arranged on or within a support structure. The microwells contain one or more active agents, alone or in combination, in one or more dosages and/or release pharmacokinetics. Preferably, the devices are configured to deliver the microdose amounts so as to virtually eliminate overlap in the tissue of active agents released from different microwells. In some embodiments, the devices are configured to facilitate implantation and retrieval in a target tissue. In an exemplary embodiment, the device has a cylindrical shape, having symmetrical wells on the outside of the device, each well containing one or more drugs, at one or more concentrations. The device is sized to permit placement using a catheter, cannula, or stylet. In a preferred embodiment, the device has a guidewire to assist in placement and retrieval. The device may also include features that assist in maintaining spatial stability of tissue excised with the device, such as fins or stabilizers that can be expanded from the device prior to or at the time of removal. Optionally, the device has fiber optics, sensors and/or interactive features such as remote accessibility (such as Wi-Fi) to provide for in situ retrieval of information and modification of device release properties. In the most preferred embodiment, the fiber optics and/or sensors are individually accessible to discrete wells.

In some embodiments, the systems provided herein are formed of biocompatible silicon, metal, ceramic or polymers. They may include materials such as radiopaque materials or materials that can be imaged using ultrasound or MRI. They can be manufactured using techniques such as deep ion etching, nano imprint lithography, micromachining, laser etching, three-dimensional printing or stereolithography. Drug can be loaded by injection of a solution or suspension into the wells followed by solvent removal by drying, evaporation, or lyophilization, or by placement of drug in tablet or particulate form into the wells. In a preferred embodiment, drugs are loaded on top of hydrogel pads within the microwells. The hydrogel pads expand during implantation to deliver the drugs to the surrounding tissue. Drug release pharmacokinetics are a function of drug solubility, excipients, dimensions of the wells, and tissue into which the device is implanted (with greater rate of release into more highly vascularized tissue, than into less vascular tissue).

In some embodiments, the systems provided herein are implanted directly into a solid tumor or tissue to be biopsied. Upon implantation, the systems provided herein locally release an array of active agents in microdoses. Subsequent analysis of tumor response to the array of active agents can be used to identify particular drugs, combinations of drugs, and/or dosages that are effective for treating a solid tumor in a patient. By locally delivering microdoses of an array of drugs, the microassay device can be used to test patients for response to large range of regimens, without inducing systemic toxicities, quickly and under actual physiological conditions. These data are used, optionally in combination with genomic data, to accurately predict systemic drug response.

Without limitation, the systems provided herein can administer an agent provided herein according to any of the methods provided herein. For example, a system provided herein can be used to deliver a microdose of an agent to a tissue in vivo. The systems described herein can provide sustained administration of a therapeutic amount of a ferroptosis-inducing agent to a tissue, wherein the sustained administration of said therapeutic amount comprises providing to said tissue the ferroptosis-inducing agent in an amount sufficient to achieve a distribution of at least about 10 ng/mm² within said tissue for a period of at least 4 hours, thereby inducing ferroptosis in the tissue. In some embodiments, the sustained administration further forms a gradient of a sub-therapeutic amount of the ferroptosis-inducing agent adjacent to the administration site within the tissue. In some embodiments, the sustained administration of a therapeutic amount of a ferroptosis-inducing agent is at least 10 hours. In some embodiments, the therapeutic amount of a ferroptosis-inducing agent is a concentration of at least about 1 μM up to 10 μM. In some embodiments, a system provided herein is implanted into a tumor. In some embodiments, the system delivers one or more a ferroptosis-inducing agents to a tumor.

### EXEMPLARY EMBODIMENTS

Provided herein are methods of inducing ferroptosis in a tissue in a subject, wherein the methods comprise: sustained administration of a therapeutic amount of a ferroptosis-inducing agent to a tissue, wherein the sustained administration of said therapeutic amount comprises providing to said tissue the ferroptosis-inducing agent in an amount sufficient to achieve a distribution of at least about 10 ng/mm² within said tissue for a period of at least 4 hours, thereby inducing ferroptosis in the tissue. Further provided herein are methods, wherein the sustained administration further forms a gradient of a sub-therapeutic amount of the ferroptosis-inducing agent adjacent to an administration site within the tissue. Further provided herein are methods, wherein the sustained administration of the ferroptosis-inducing agent comprises additional administration steps. Further provided herein are methods, wherein the tissue comprises a heterogeneous population of cells, wherein the heterogeneous population of cells comprises at least one of precancerous cells and non-cancerous cells. Further provided herein are methods, wherein the tissue comprises a heterogeneous population of cells, wherein the heterogeneous population of cells comprises a population of immune cells. Further provided herein are methods, wherein the tissue comprises a heterogeneous population of cells, wherein the heterogeneous population of cells comprises a first population of cells comprising a greater concentration of selenium or iron compared to a predetermined level of selenium or iron; and a second population of cells comprising a normal concentration of selenium or iron compared to said predetermined level of selenium or iron. Further provided herein are methods, wherein the tissue comprises a homogenous population of cells. Further provided herein are methods, wherein the tissue comprises a plurality of cancer cells. Further provided herein are methods, wherein the tissue comprises a plurality of cells expressing one or more markers indicative of a mesenchymal state. Further provided herein are methods, wherein the one or more markers are selected from the group consisting of. ZEB1, ACSL4, FADS2, PPARγ, Fsp1, SLC7A11, SLC3A2, and LPCAT3. Further provided herein are methods, wherein the tissue comprises a plurality of cells that have a reduction in the expression of one or more endothelial cell markers. Further provided herein are methods, wherein the endothelial cell marker is vimentin, E-cadherin, or beta (f)-actin. Further provided herein are methods, wherein the tissue comprises a histological abnormality. Further provided herein are methods, wherein the histological abnormality is determined by a tissue biopsy prior to or during the targeted, sustained administration of the ferroptosis-inducing agent to the tissue. Further provided herein are methods, wherein the histological abnormality is hyperplasia or fibrosis. Further provided herein are methods, wherein the tissue comprises a plurality of cells with a polyunsaturated fatty acids (PUFA) concentration greater than a PUFA concentration in cells of a normal tissue. Further provided herein are methods, wherein the PUFA concentration in the plurality of greater than a predetermined PUFA concentration. Further provided herein are methods, wherein the tissue comprises a plurality of cells with a peroxidizability index (PI) greater than the PI in cells of normal or healthy tissue; and ferroptosis is induced in the plurality of cells. Further provided herein are methods, wherein the PI in the plurality of cells is greater than a predetermined PI. Further provided herein are methods, wherein the ferroptosis-inducing agent is an inhibitor of glutathione peroxidase 4 (GPX4), glutathione synthetase, glutamate-cysteine ligase, phosphoseryl-TRNA Kinase (PSTK), Eukaryotic Elongation Factor Selenocysteine-TRNA Specific (EEFSEC), Selenophosphate Synthetase 2 (SEPHS2), Sep (O-Phosphoserine) TRNA:Sec (Selenocysteine) TRNA Synthase (SEPSECS), or SECIS Binding Protein 2 (SECISBP2). Further provided herein are methods, wherein the inhibitor is a small molecule, a peptide, or a nucleic acid. Further provided herein are methods, wherein the ferroptosis-inducing agent is any one or more of the agents in Table 1. Further provided herein are methods, wherein the ferroptosis-inducing agent is any one or more of the agents in Table 2 or Table 3. Further provided herein are methods, wherein the ferroptosis-inducing agent is selected from Table 1, for instance, from the group consisting of. (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, analogs, or derivatives thereof. Further provided herein are methods, wherein the ferroptosis-inducing agent contacts the tissue at a localized site for about 6 hours. Further provided herein are methods, wherein the ferroptosis-inducing agent contacts the tissue at a localized site for about 10 hours. Further provided herein are methods, wherein the ferroptosis-inducing agent contacts the tissue at a localized site for about 24 hours. Further provided herein are methods, wherein the ferroptosis-inducing agent contacts the tissue at a localized site for about 48 hours. Further provided herein are methods, wherein the ferroptosis-inducing agent contacts the tissue at a localized site for about 72 hours. Further provided herein are methods, wherein the ferroptosis-inducing agent is administered at a concentration of at least about 1 μM to 10 μM. Further provided herein are methods, wherein the tissue is resistant to treatment with an anti-apoptotic agent. Further provided herein are methods, wherein the tissue is a tumor or a tissue comprising a plurality of cancer cells. Further provided herein are methods, wherein the cancer is a solid tumor or a blood cancer. Further provided herein are methods, wherein the blood cancer is a leukemia or a lymphoma. Further provided herein are methods, wherein the solid tumor is a carcinoma, a melanoma, or a sarcoma. Further provided herein are methods, wherein the melanoma is a dedifferentiated melanoma or amelanotic melanoma. Further provided herein are methods, wherein the subject has or is at risk of developing cancer. Further provided herein are methods, wherein the cancer is acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CIVIL, T cell CML), and chronic lymphocytic leukemia (CLL) (e.g., B cell CLL, T cell CLL)); lymphoma such as Hodgkin lymphoma (HL) (e.g., B cell HL, T cell HL) and non-Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma, a liver carcinoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), angiogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); colorectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; or vulvar cancer (e.g., Paget's disease of the vulva).

Further provided herein are methods of inducing iron-dependent cell death in a tissue in a subject, wherein the methods comprise: contacting a tissue in vivo with an effective amount of an iron-dependent cell death agent for a duration of time of at least 4 hours, wherein the tissue comprises one or more of: (a) a plurality of cells comprising a concentration of selenium greater than a selenium concentration in a corresponding normal tissue; (b) a plurality of cells comprising a concentration of iron greater than an iron concentration in a corresponding normal tissue; (c) a plurality of cells comprising a PUFA concentration greater than a PUFA concentration in a corresponding normal tissue; (d) a plurality of cells expressing one or more markers indicative of a mesenchymal state; and/or (e) a plurality of cells comprising a peroxidizability index (PI) greater than a PI in a corresponding normal tissue, wherein the effective amount of the iron-dependent cell death agent is a concentration of at least about 0.1 μM up to 500 μM in the tissue for the duration of time. Further provided herein are methods, wherein the iron-dependent cell death agent is any one of the agents listed in Table 1. Further provided herein are methods, wherein the iron-dependent cell death agent is any one of the agents listed in Table 2 or Table 3. Further provided herein are methods, wherein the ferroptosis-inducing agent is selected from Table 1, for instance, from the group consisting of: (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, analogs, or derivatives thereof. Further provided herein are methods, wherein the iron-dependent cell death agent contacts the tissue at a localized site for about 6 hours. Further provided herein are methods, wherein the iron-dependent cell death agent contacts the tissue at a localized site for about 10 hours. Further provided herein are methods, wherein the iron-dependent cell death agent contacts the site on the tumor for about 24 hours. Further provided herein are methods, wherein the iron-dependent cell death agent contacts the site on the tumor for about 48 hours. Further provided herein are methods, wherein the iron-dependent cell death agent contacts the site on the tumor for about 72 hours. Further provided herein are methods, wherein the tissue is a tumor or a pre-cancerous lesion. Further provided herein are methods, wherein the tumor is resistant to one or more anti-apoptosis agents. Further provided herein are methods, wherein the tumor is a carcinoma, a melanoma, or a sarcoma. Further provided herein are methods, wherein the melanoma is a dedifferentiated melanoma or a amelanotic melanoma. Further provided herein are methods, wherein the method further comprises a step of obtaining a biopsy of the tissue for histological analysis. Further provided herein are methods, wherein the tissue comprises a histological abnormality, wherein the histological abnormality is hyperplasia or fibrosis. Further provided herein are methods, wherein the one or more markers indicative of a mesenchymal state are selected from the group consisting of: ZEB1, ACSL4, FADS2, PPARγ, Fsp1, SLC7A11, SLC3A2, and LPCAT3. Further provided herein are methods, wherein the tissue comprises a plurality of cells that have a reduction in the expression of one or more endothelial cell markers. Further provided herein are methods, wherein the endothelial cell marker is vimentin, E-cadherin, or beta (p)-actin. Further provided herein are methods, wherein the agent reduces tissue size or tissue volume by at least 5%. Further provided herein are methods, wherein the agent is administered with one additional agent. Further provided herein are methods, wherein the additional agent is a cell death-inducing agent or a dietary supplement.

Further provided herein are methods of inducing targeted cell death in a mammalian tissue in vivo, wherein the methods comprise: (a) contacting a mammalian tissue with a priming agent; (b) contacting the mammalian tissue in vivo with an effective amount of a ferroptosis-inducing agent for a duration of time of at least 4 hours, when a plurality of cells within the mammalian tissue are responsive to the priming agent as determined by detecting in the mammalian tissue: (i) a plurality of cells comprising a concentration of selenium greater than a selenium concentration in the mammalian tissue prior to contacting with the priming agent; (ii) a plurality of cells comprising a concentration of iron greater than an iron concentration in the mammalian tissue prior to contacting with the priming agent; (iii) a plurality of cells comprising a PUFA concentration greater than a PUFA concentration in the mammalian tissue prior to contacting with the priming agent; (iv) a plurality of cells expressing one or more markers indicative of a mesenchymal state; (v) a plurality of cells comprising a peroxidizability index (PI) greater than a PI in the mammalian tissue prior to contacting with the priming agent; and/or (vi) hyperproliferation of cells in the mammalian tissue, wherein the ferroptosis-inducing agent induces targeted cell death in the mammalian tissue in vivo. Further provided herein are methods, wherein step (a) is performed, in vivo, in vitro, or ex vivo. Further provided herein are methods, wherein the methods further comprise a step of obtaining a biopsy of the mammalian tissue for histological analysis. Further provided herein are methods, wherein the methods further comprise a step of detecting a plurality of cells within the mammalian tissue as responsive to the priming agent. Further provided herein are methods, wherein the detecting is via a histological assay or an immunohistochemical assay. Further provided herein are methods, wherein the priming agent is any one of the agents listed in Table 4. Further provided herein are methods, wherein the priming agent is priming agent is liproxstatin-1, ferrostatin-1, deferoxamine (DFO), iron, vitamin E, a polyunsaturated fatty acid, or selenium. Further provided herein are methods, wherein the methods further comprise administering a cell death-inducing agent. Further provided herein are methods, wherein the cell-death inducing agent is a chemotherapeutic agent. Further provided herein are methods, wherein the ferroptosis-inducing agent is any one of the agents listed in Table 1. Further provided herein are methods, wherein the ferroptosis-inducing agent is selected from Table 1, for instance, from the group consisting of (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO₂, CIL56, dihydroisotanshinone I, GPX4-IN-3, analogs, or derivatives thereof. Further provided herein are methods, wherein the ferroptosis-inducing agent contacts the mammalian tissue for about 6 hours. Further provided herein are methods, wherein the ferroptosis-inducing agent contacts the mammalian tissue for about 10 hours. Further provided herein are methods, wherein the ferroptosis-inducing agent contacts the mammalian tissue for about 24 hours. Further provided herein are methods, wherein the ferroptosis-inducing agent contacts the mammalian tissue for about 48 hours. Further provided herein are methods, wherein the ferroptosis-inducing agent contacts the mammalian tissue for about 72 hours. Further provided herein are methods, wherein the effective amount of the ferroptosis-inducing agent is a concentration of at least about 1 μM to 10 μM. Further provided herein are methods, wherein following contact with a ferroptosis-inducing agent, cell death can be detected at a time point at or after contacting the mammalian tissue with the ferroptosis-inducing agent. Further provided herein are methods, wherein following contact with a ferroptosis-inducing agent, immune cell recruitment can be detected at a time point at or after contacting the mammalian tissue with the ferroptosis-inducing agent. Further provided herein are methods, wherein the tissue is human tissue. Further provided herein are methods, wherein the administering or contacting step is via intratumoral injection, oral administration, transdermal injection, inhalation, nasal administration, topical administration, vaginal administration, ophthalmic administration, intracerebral administration, rectal administration. Further provided herein are methods, wherein the administering or contacting step is via intravenous administration, intra-arterial administration, intramuscular administration, or subcutaneous administration.

Further provided herein are systems, wherein the systems comprise: an implantable microdevice configured for localized administration to a tissue comprising: (a) a cylindrical support structure having at least one microwell on a surface of or formed within the support structure; (b) a microdose of a ferroptosis-inducing agent in the at least one microwell; and (c) a compound release mechanism for sustained administration for controlling a release of the ferroptosis-inducing agent from the microwell, wherein the microdose of the ferroptosis-inducing agent forms a gradient of a sub-therapeutic dose of the ferroptosis-inducing agent an administration site within the tissue for a duration of time of at least 4 hours, wherein the microdevice is configured to permit implantation into the tissue using a catheter, cannula or biopsy needle, and wherein the microdevice is further configured to release the ferroptosis-inducing agent from the at least one microwell to the administration site within the apoptosis-resistant tissue adjacent to the at least one microwell.

Further provided herein are systems for screening for ferroptosis-induced cell death in vivo, the systems comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) at least one microwell containing one or more ferroptosis inhibitors; (ii) a micro-dose of the one or more active agents; and/or one or more ferroptosis inhibitors in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, wherein the system measures an outcome of ferroptosis induction in the animal model after administration of the one or more active agents into the tissue relative to administration of the one or more active agents and one or more ferroptosis inhibitors, and identifying one or more active agents induces ferroptosis in the tissue.

Further provided herein are systems for screening for ferroptosis-induced cell death in vivo, the systems comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) at least one microwell containing one or more ferroptosis inhibitors; (ii) a micro-dose of the one or more active agents; and/or one or more ferroptosis inhibitors in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, wherein the system measures an outcome of ferroptosis induction in the animal model after administration of the one or more active agents into the tissue relative to administration of the one or more active agents and one or more ferroptosis inhibitors, and identifying one or more active agents induces ferroptosis in the tissue. Further provided herein are systems, wherein the ferroptosis inhibitor is liproxstatin-1 or ferrostatin-1.

Further provided herein are methods of modulating ferroptosis in vivo, the methods comprising: (a) contacting a mammalian tissue in vivo with an effective amount of a ferroptosis-inducing agent for a duration of time of at least 4 hours, wherein the ferroptosis-inducing agent induces targeted cell death in the mammalian tissue in vivo; and (b) contacting the mammalian tissue in vivo with an effective amount of a ferroptosis-inducing agent and a ferroptosis inhibitor, thereby modulation ferroptosis in vivo. Further provided herein are methods, wherein the ferroptosis inhibitor is liproxstatin-1, ferrostatin-1, deferoxamine (DFO), iron, vitamin E, a polyunsaturated fatty acid, or selenium. Further provided herein are methods, wherein the ferroptosis-inducing agent is an inhibitor of glutathione peroxidase 4 (GPX4), glutathione synthetase, glutamate-cysteine ligase, phosphoseryl-TRNA Kinase (PSTK), Eukaryotic Elongation Factor Selenocysteine-TRNA Specific (EEFSEC), Selenophosphate Synthetase 2 (SEPHS2), Sep (O-Phosphoserine) TRNA:Sec (Selenocysteine) TRNA Synthase (SEPSECS), or SECIS Binding Protein 2 (SECISBP2). Further provided herein are methods, wherein the ferroptosis-inducing agent is selected from the group consisting of (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, analogs, or derivatives thereof. Further provided herein are methods, wherein the ferroptosis-inducing agent contacts the mammalian tissue for about 6 hours. Further provided herein are methods, wherein the ferroptosis-inducing agent contacts the mammalian tissue for about 10 hours. Further provided herein are methods, wherein the ferroptosis-inducing agent contacts the mammalian tissue for about 24 hours. Further provided herein are methods, wherein the ferroptosis-inducing agent contacts the mammalian tissue for about 48 hours. Further provided herein are methods, wherein the ferroptosis-inducing agent contacts the mammalian tissue for about 72 hours. Further provided herein are methods, wherein the effective amount of the ferroptosis-inducing agent is a concentration of at least about 1 μM to 10 μM. Further provided herein are methods, wherein following contact with a ferroptosis-inducing agent, cell death can be detected at a time point at or after contacting the mammalian tissue with the ferroptosis-inducing agent. Further provided herein are methods, wherein following contact with a ferroptosis-inducing agent, immune cell recruitment can be detected at a time point at or after contacting the mammalian tissue with the ferroptosis-inducing agent. Further provided herein are methods, wherein the tissue is human tissue. Further provided herein are methods, wherein the administering or contacting step is via intratumoral injection, oral administration, transdermal injection, inhalation, nasal administration, topical administration, vaginal administration, ophthalmic administration, intracerebral administration, rectal administration. Further provided herein are methods, wherein the administering or contacting step is via intravenous administration, intra-arterial administration, intramuscular administration, or subcutaneous administration. Further provided herein are methods, wherein the method further comprises measuring one or more parameters indicative of ferroptosis in the mammalian tissue, wherein the one or parameters are selected from: concentration of selenium; concentration of iron; PUFA concentration; expression one or more markers indicative of a mesenchymal state; peroxidizability index (PI); and/or cell proliferation.

Further provided herein are compositions for the treatment of a disease or disorder, wherein the compositions comprise any one of the agents in Table 1 or a combination of agents; and a system provided herein.

Further provided herein are pharmaceutical compositions for the treatment of a disease or disorder, wherein the pharmaceutical compositions comprise any one of the agents in Table 1 or a combination of agents; and a pharmaceutically acceptable excipient. Further provided herein are pharmaceutical compositions for the treatment of a disease or disorder, wherein the pharmaceutical compositions comprise any one of the agents in Table 1, Table 2, or a combination of agents; and a pharmaceutically acceptable excipient.

In some embodiments is a compound, a diastereomer or an enantiomer of the compound, or a pharmaceutically acceptable salt of any of the foregoing, or a deuterated derivative of any of the foregoing; wherein the compound is selected from the group consisting of:

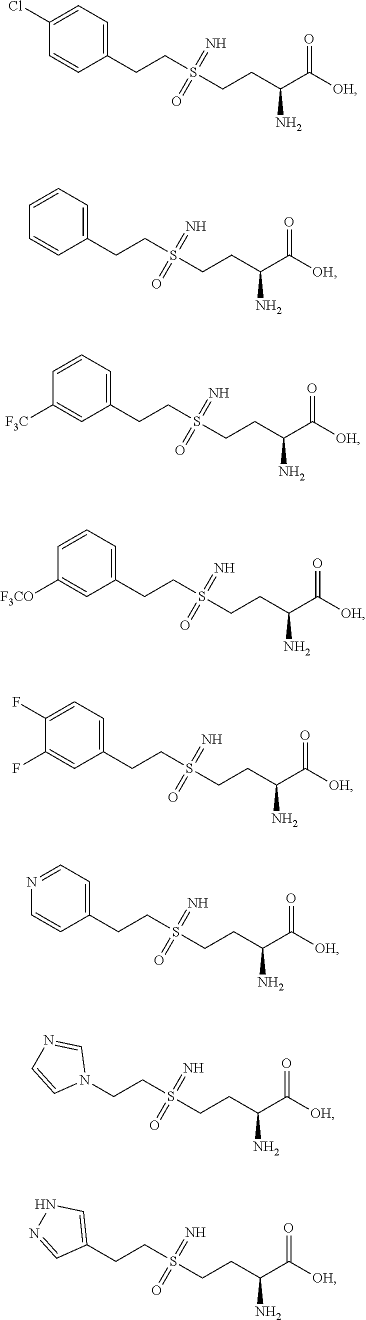

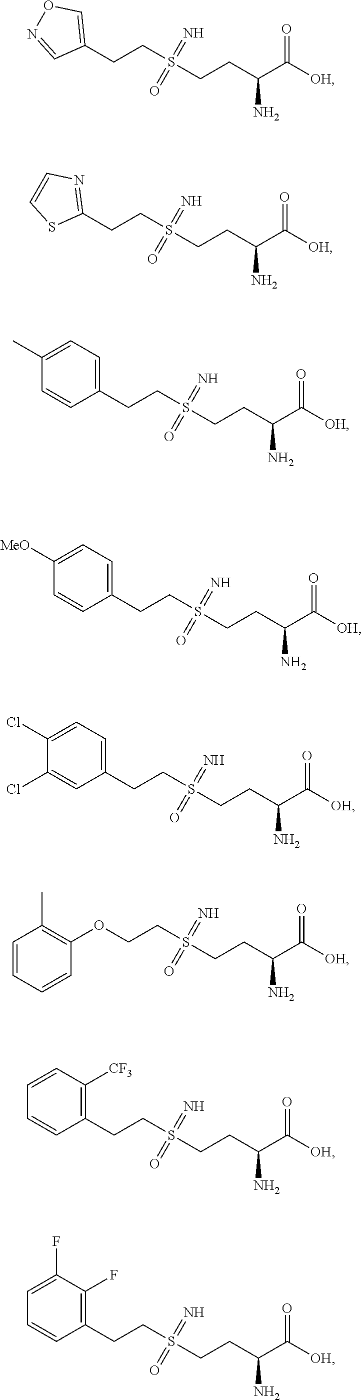

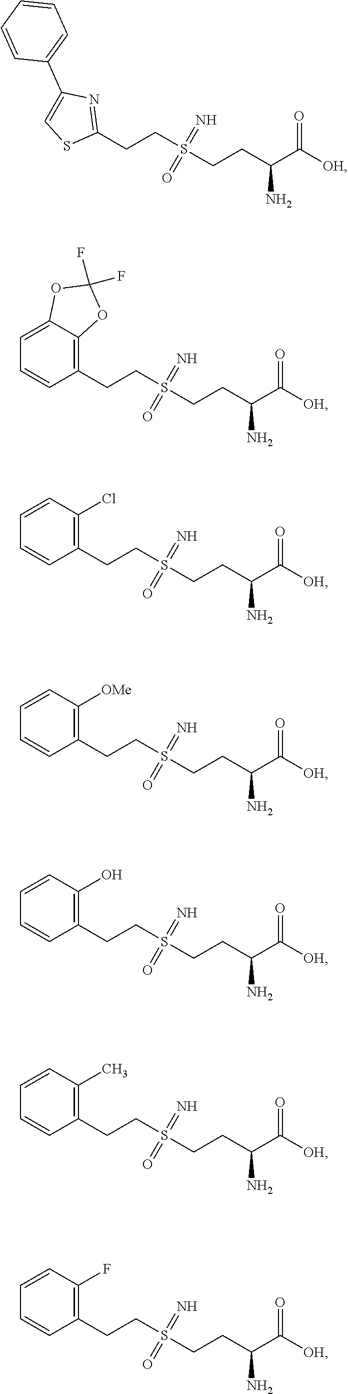

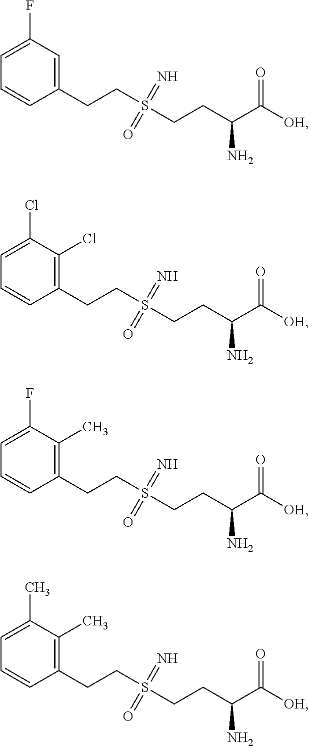 [SMILES: N=S(=O)(CCc1cccc(F)c1)CC[C@H](N)C(=O)O ; N=S(=O)(CCc1cccc(Cl)c1Cl)CC[C@H](N)C(=O)O ; Cc1c(F)cccc1CCS(=N)(=O)CC[C@H](N)C(=O)O ; Cc1cccc(CCS(=N)(=O)CC[C@H](N)C(=O)O)c1C]

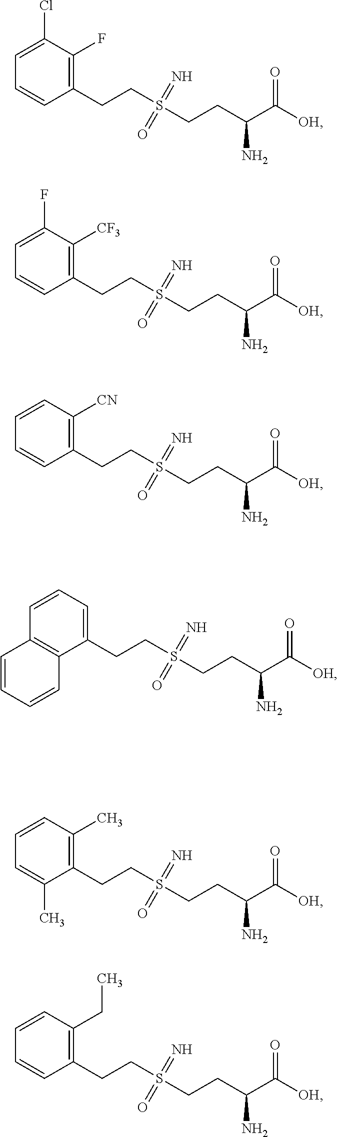 [SMILES: N=S(=O)(CCc1cccc(Cl)c1F)CC[C@H](N)C(=O)O ; N=S(=O)(CCc1cccc(F)c1C(F)(F)F)CC[C@H](N)C(=O)O ; N#Cc1ccccc1CCS(=N)(=O)CC[C@H](N)C(=O)O ; N=S(=O)(CCc1cccc2ccccc12)CC[C@H](N)C(=O)O ; Cc1cccc(C)c1CCS(=N)(=O)CC[C@H](N)C(=O)O ; CCc1ccccc1CCS(=N)(=O)CC[C@H](N)C(=O)O]

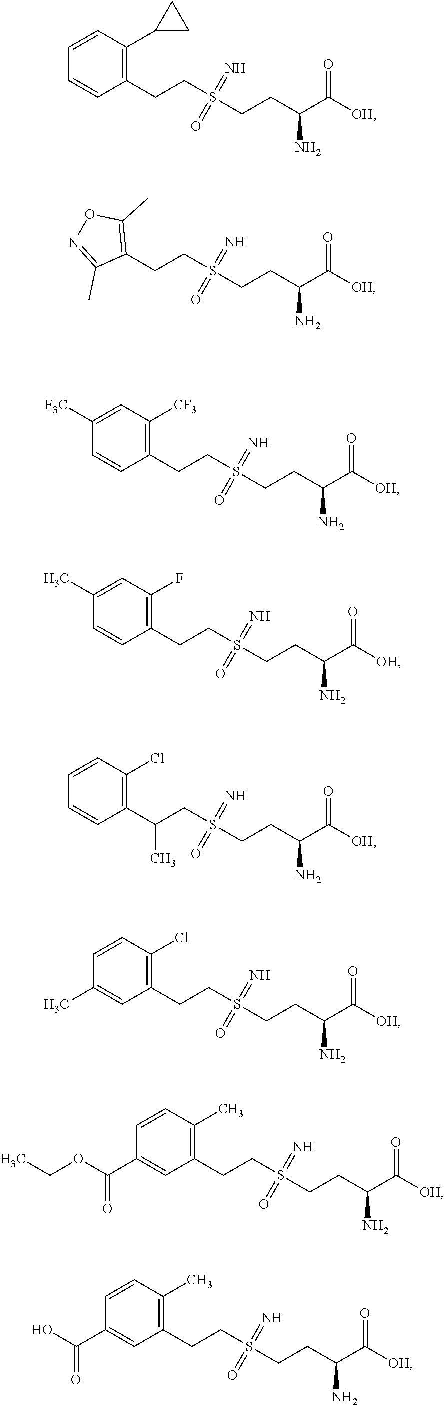

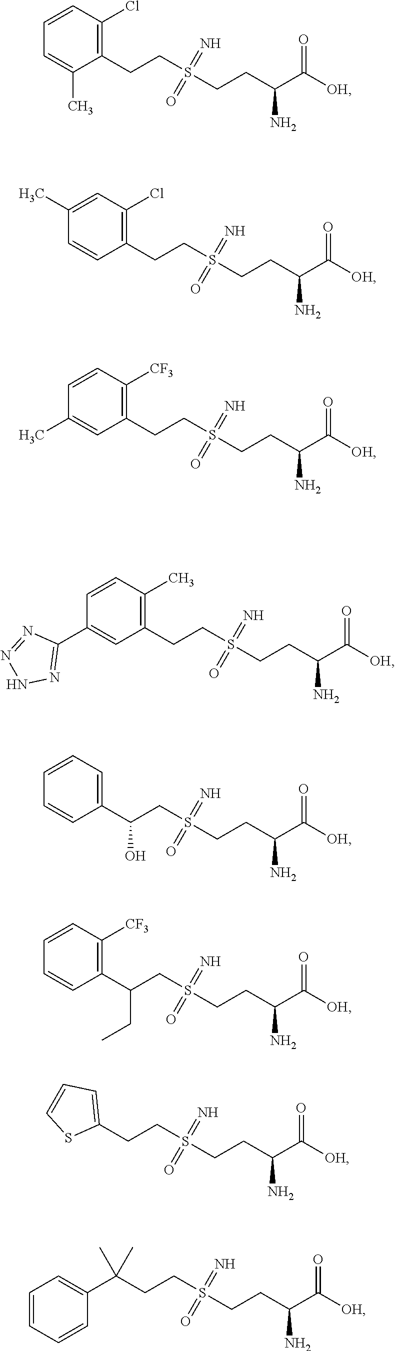

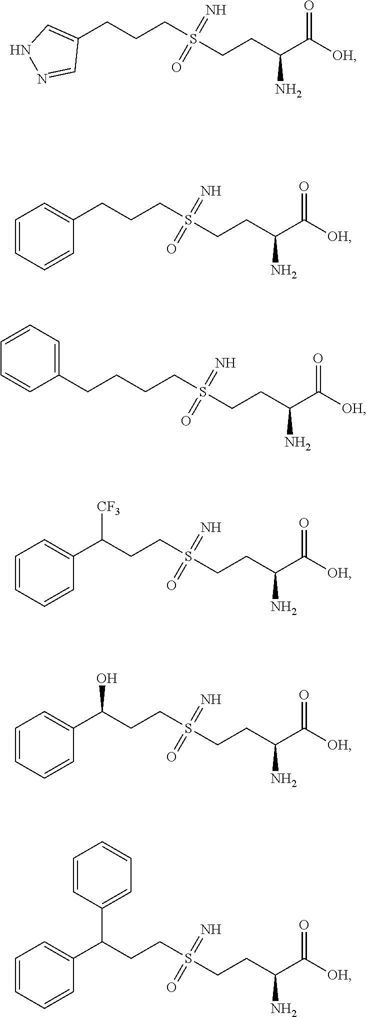 [SMILES: N=S(=O)(CCCc1cn[nH]c1)CC[C@H](N)C(=O)O ; N=S(=O)(CCCc1ccccc1)CC[C@H](N)C(=O)O ; N=S(=O)(CCCCc1ccccc1)CC[C@H](N)C(=O)O ; N=S(=O)(CCC(c1ccccc1)C(F)(F)F)CC[C@H](N)C(=O)O ; N=S(=O)(CC[C@H](N)C(=O)O)CC[C@H](O)c1ccccc1 ; N=S(=O)(CCC(c1ccccc1)c1ccccc1)CC[C@H](N)C(=O)O]

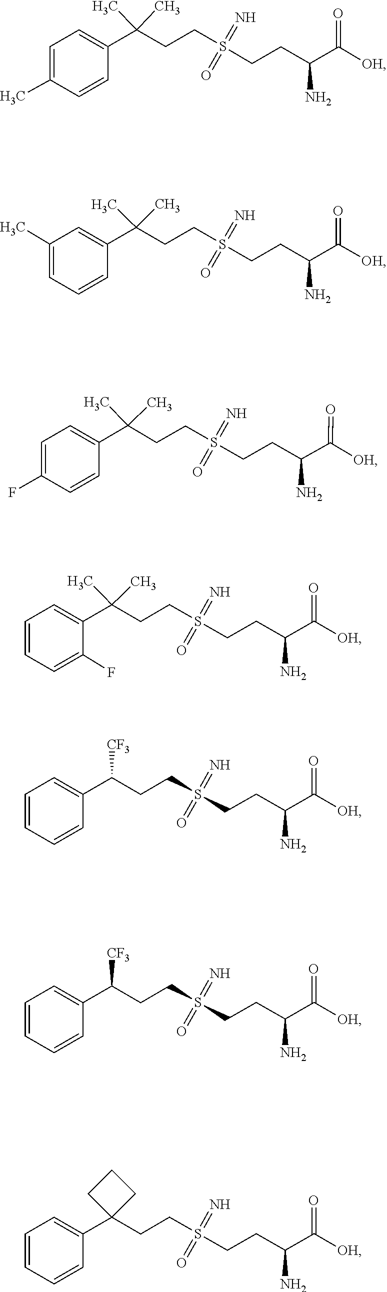

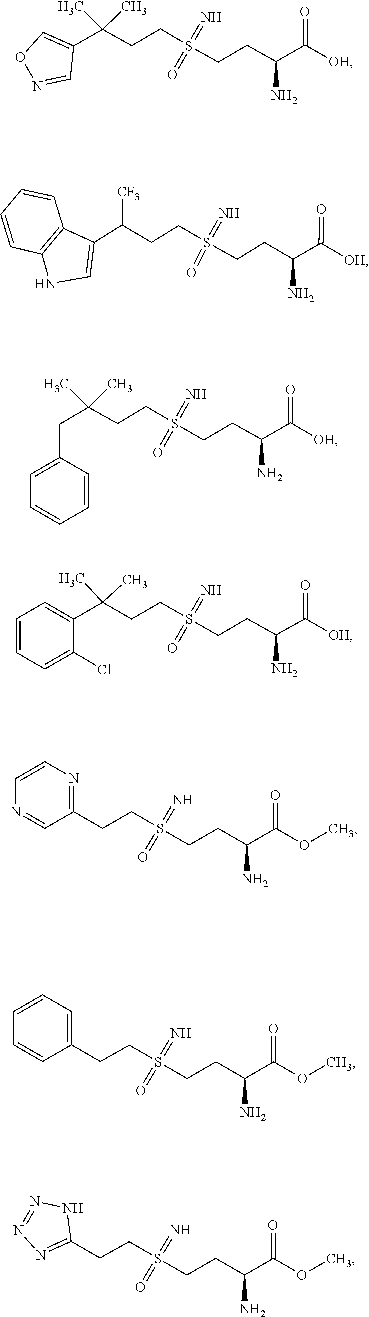

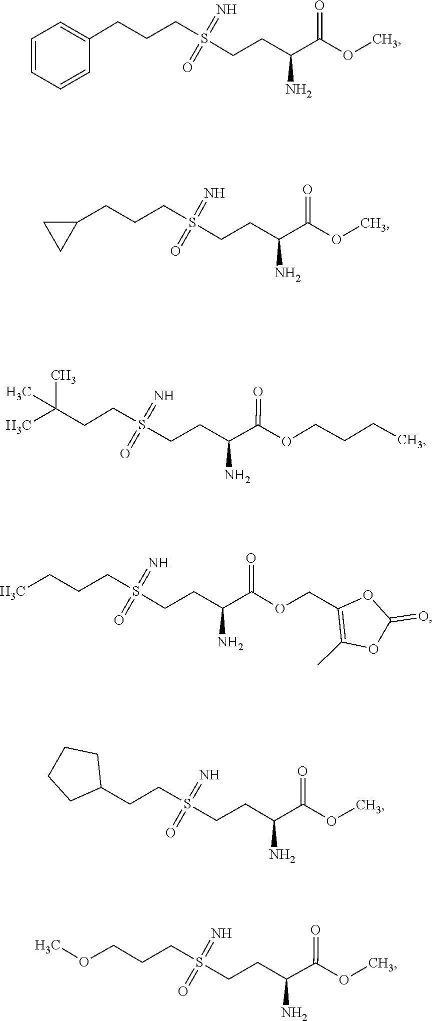 [SMILES: COC(=O)[C@@H](N)CCS(=N)(=O)CCCc1ccccc1 ; COC(=O)[C@@H](N)CCS(=N)(=O)CCCC1CC1 ; CCCCOC(=O)[C@@H](N)CCS(=N)(=O)CCC(C)(C)C ; CCCCS(=N)(=O)CC[C@H](N)C(=O)OCc1oc(=O)oc1C ; COC(=O)[C@@H](N)CCS(=N)(=O)CCC1CCCC1 ; COCCCS(=N)(=O)CC[C@H](N)C(=O)OC]

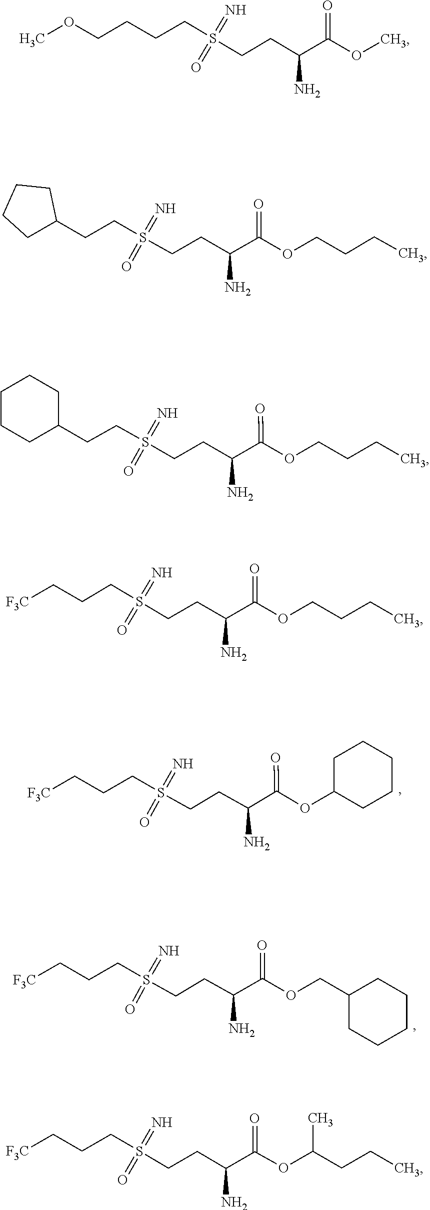

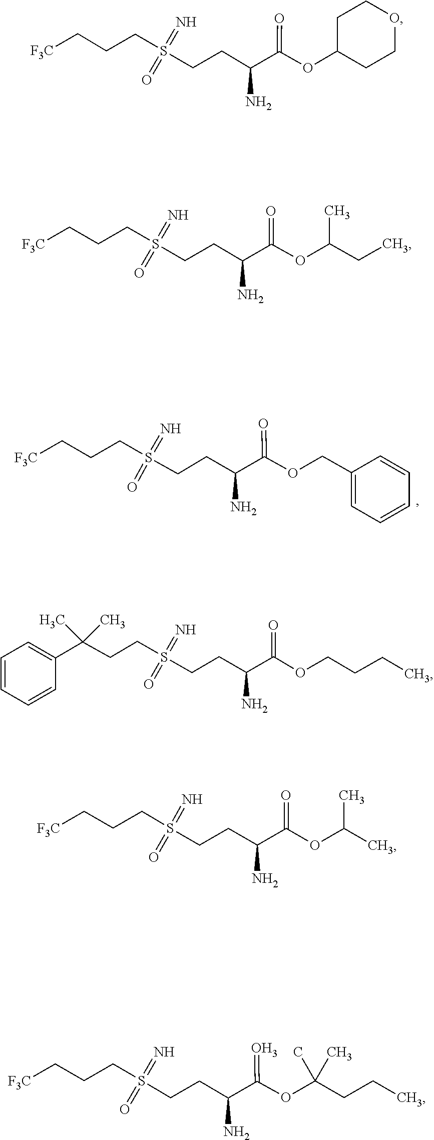

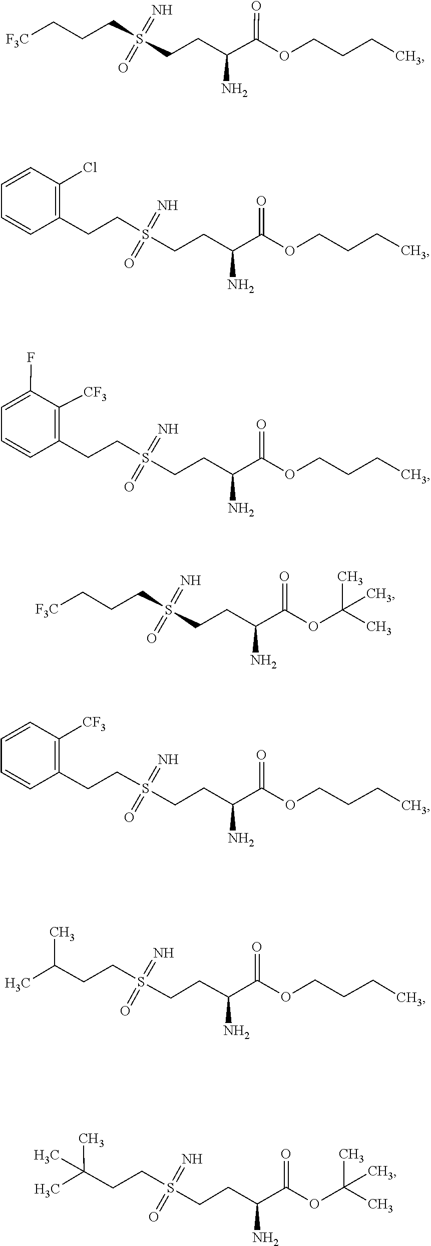

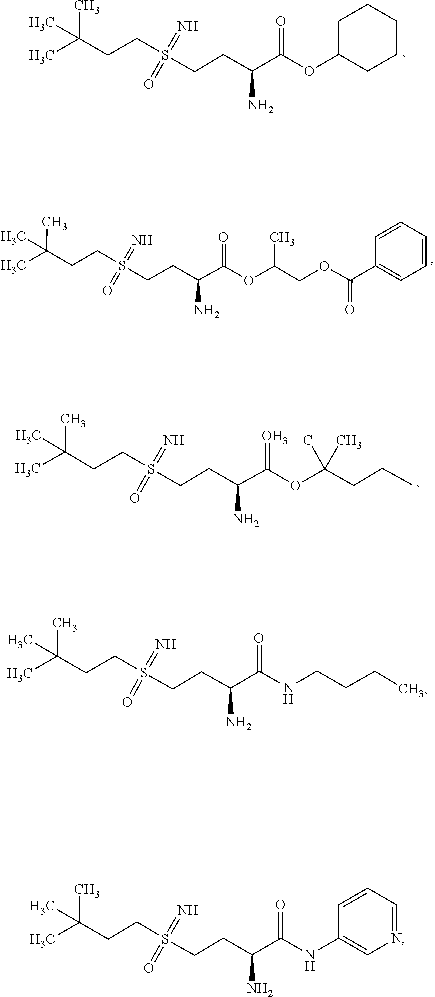

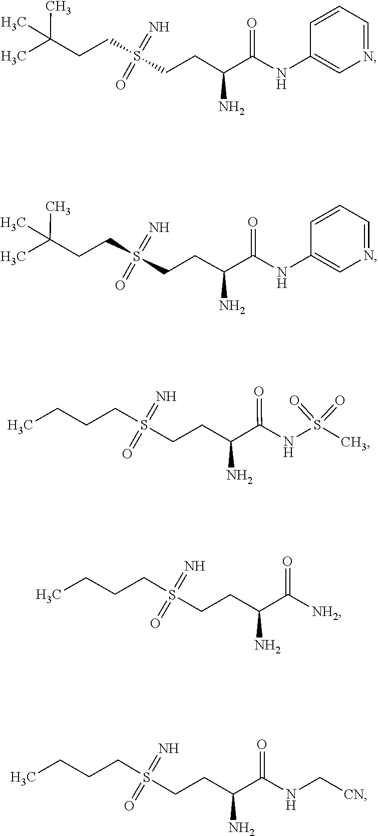 [SMILES: CC(C)(C)CC[S@@](=N)(=O)CC[C@H](N)C(=O)Nc1cccnc1 ; CC(C)(C)CC[S@](=N)(=O)CC[C@H](N)C(=O)Nc1cccnc1 ; CCCCS(=N)(=O)CC[C@H](N)C(=O)NS(C)(=O)=O ; CCCCS(=N)(=O)CC[C@H](N)C(N)=O ; CCCCS(=N)(=O)CC[C@H](N)C(=O)NCC#N]

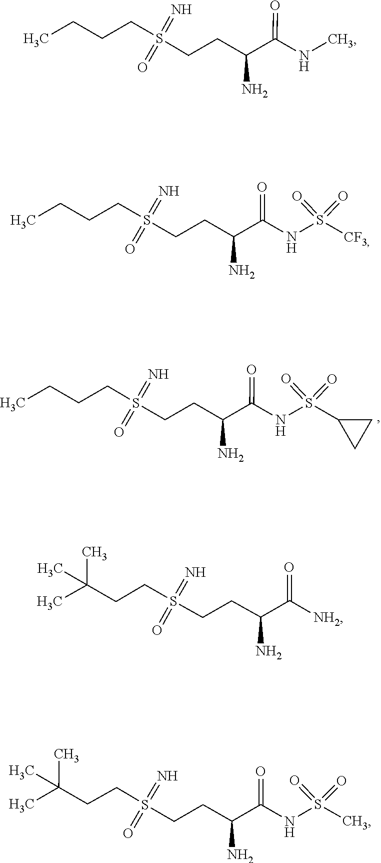 [SMILES: CCCCS(=N)(=O)CC[C@H](N)C(=O)NC ; CCCCS(=N)(=O)CC[C@H](N)C(=O)NS(=O)(=O)C(F)(F)F ; CCCCS(=N)(=O)CC[C@H](N)C(=O)NS(=O)(=O)C1CC1 ; CC(C)(C)CCS(=N)(=O)CC[C@H](N)C(N)=O ; CC(C)(C)CCS(=N)(=O)CC[C@H](N)C(=O)NS(C)(=O)=O]

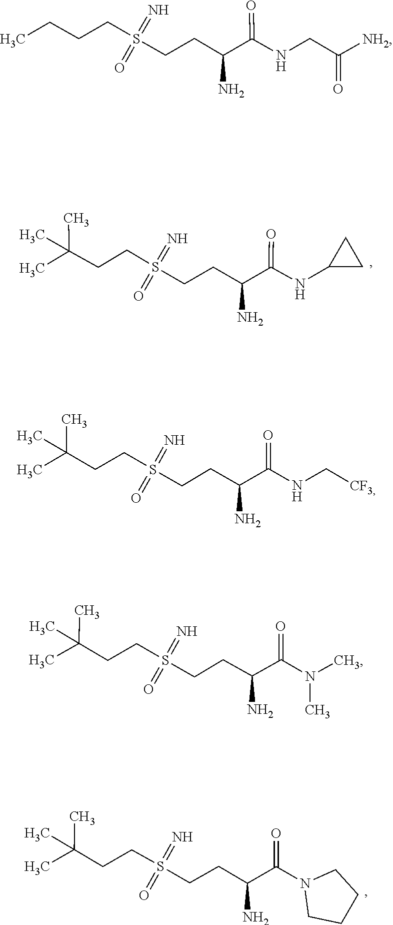 [SMILES: CCCCS(=N)(=O)CC[C@H](N)C(=O)NCC(N)=O ; CC(C)(C)CCS(=N)(=O)CC[C@H](N)C(=O)NC1CC1 ; CC(C)(C)CCS(=N)(=O)CC[C@H](N)C(=O)NCC(F)(F)F ; CN(C)C(=O)[C@@H](N)CCS(=N)(=O)CCC(C)(C)C ; CC(C)(C)CCS(=N)(=O)CC[C@H](N)C(=O)N1CCCC1]

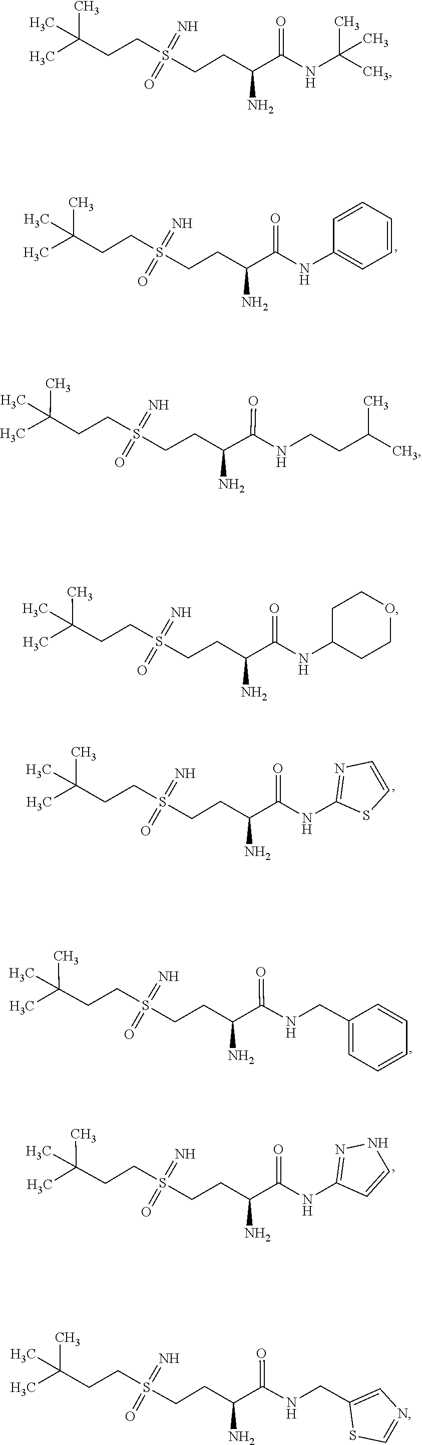

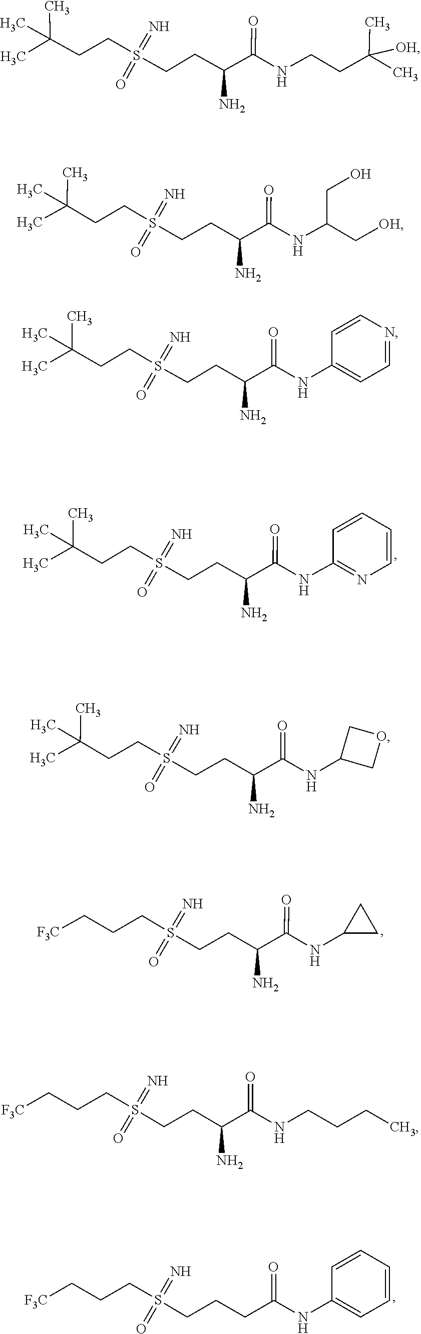

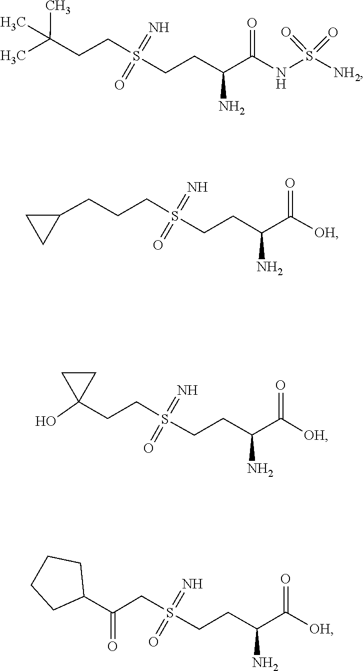 [SMILES: CC(C)(C)CCS(=N)(=O)CC[C@H](N)C(=O)NS(N)(=O)=O ; N=S(=O)(CCCC1CC1)CC[C@H](N)C(=O)O ; N=S(=O)(CC[C@H](N)C(=O)O)CCC1(O)CC1 ; N=S(=O)(CC[C@H](N)C(=O)O)CC(=O)C1CCCC1]

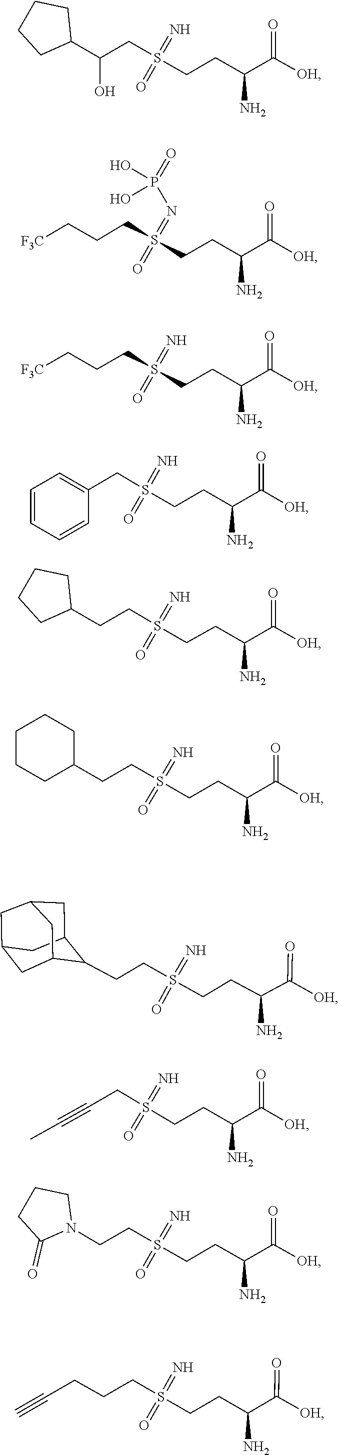

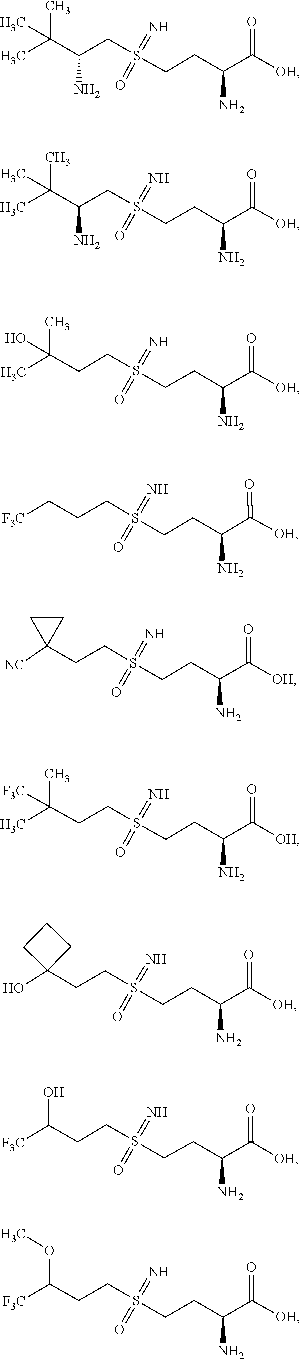

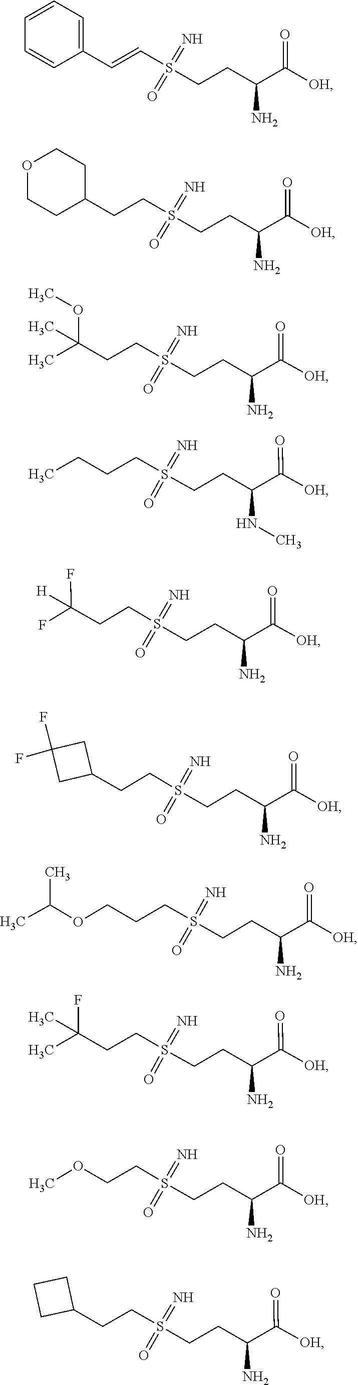

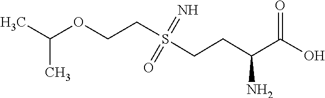 [SMILES: CC(C)OCCS(=N)(=O)CC[C@H](N)C(=O)O]

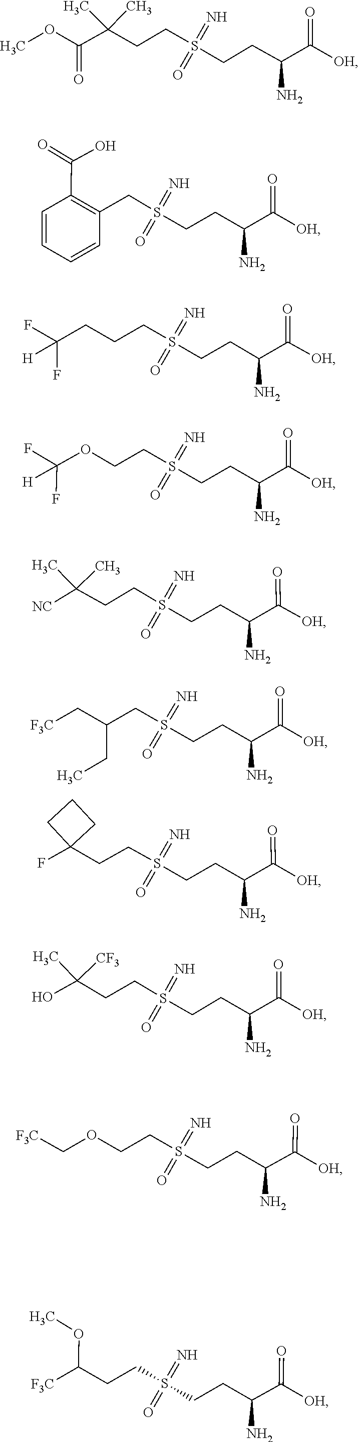

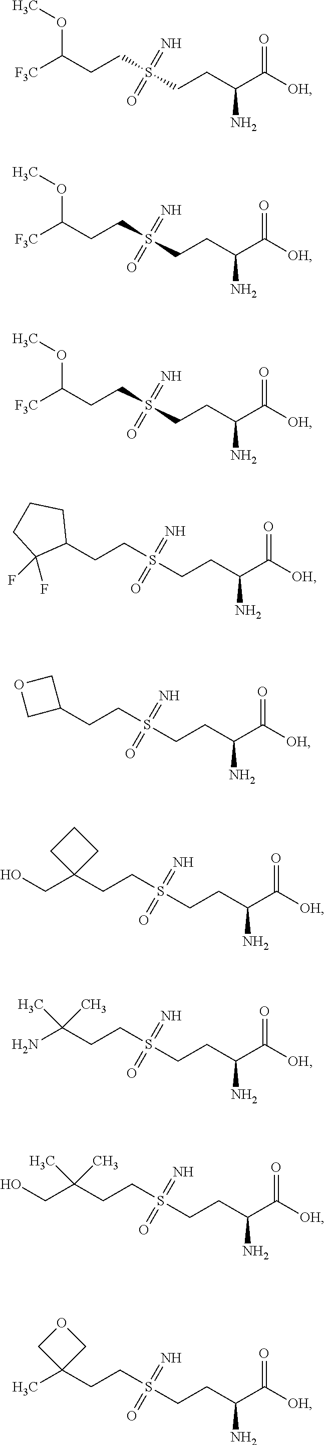

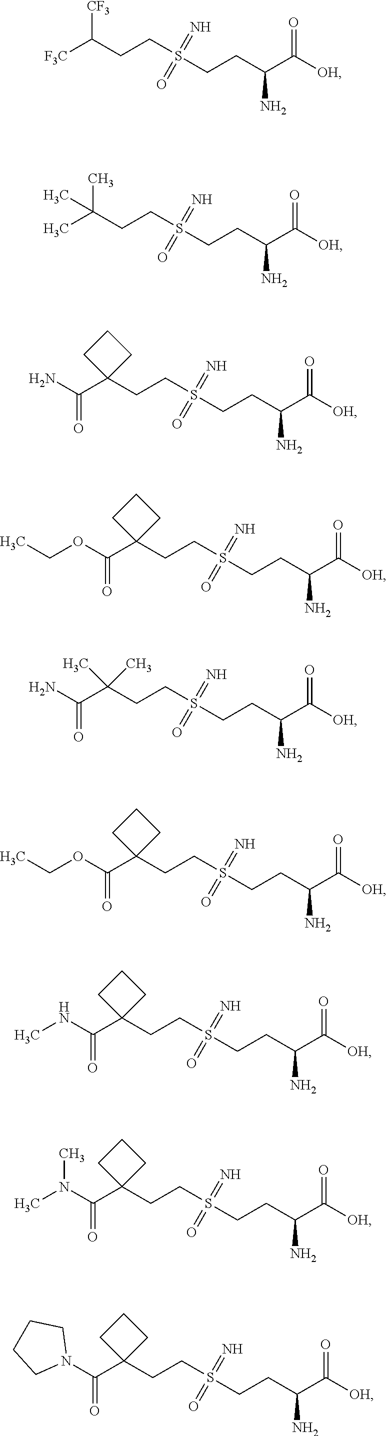

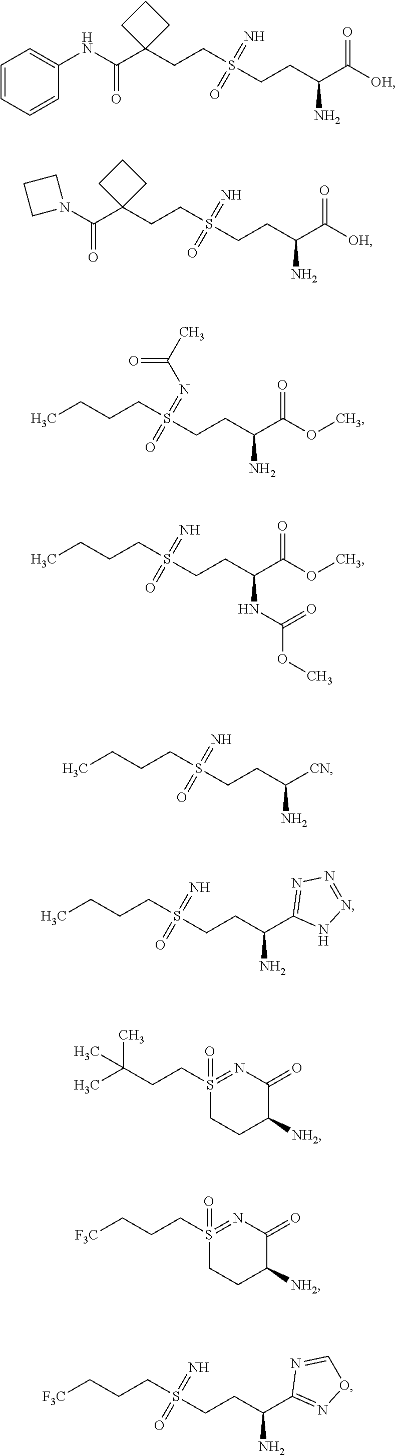

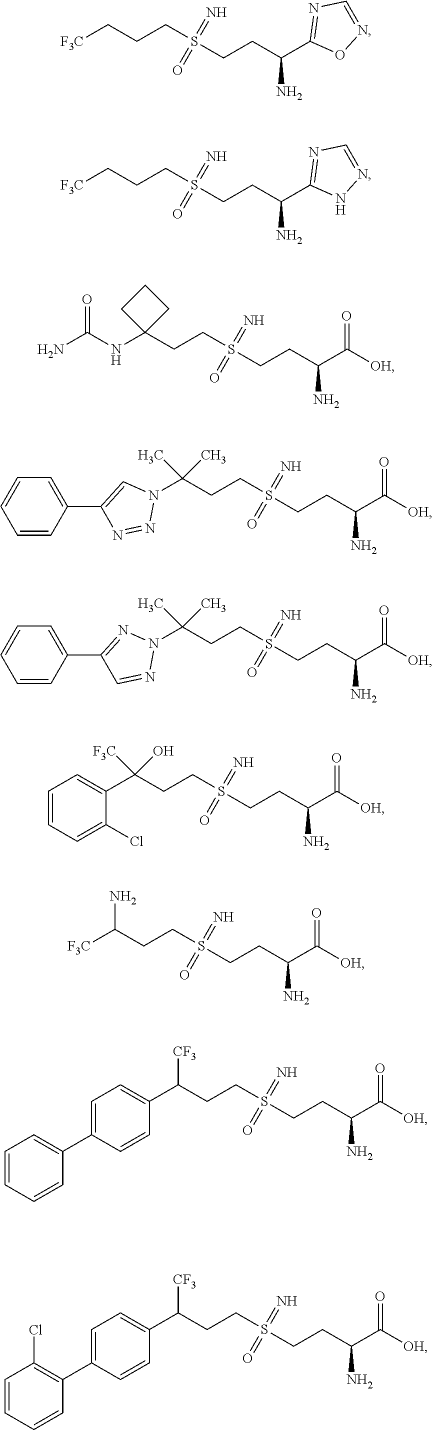

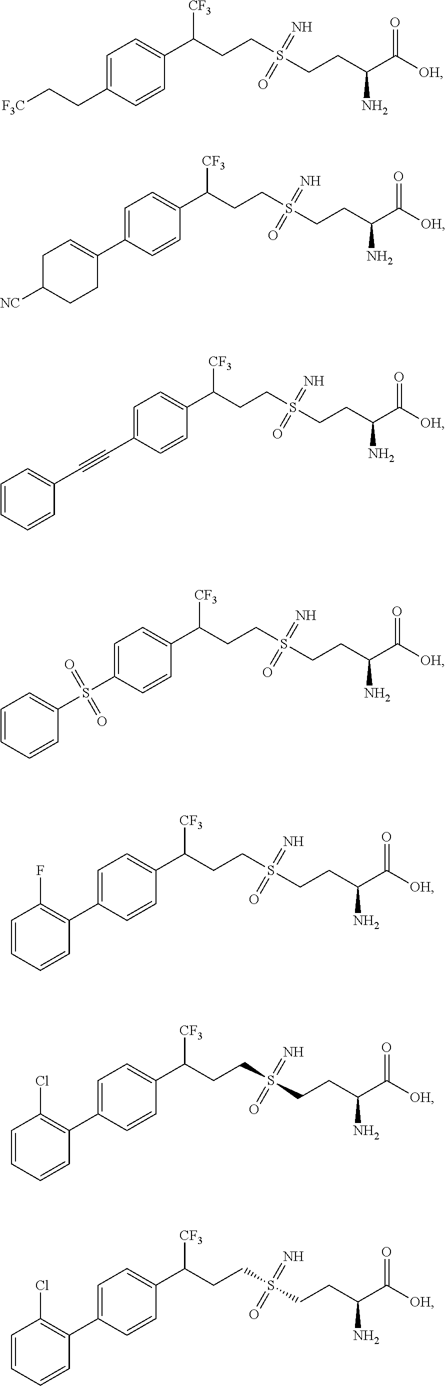

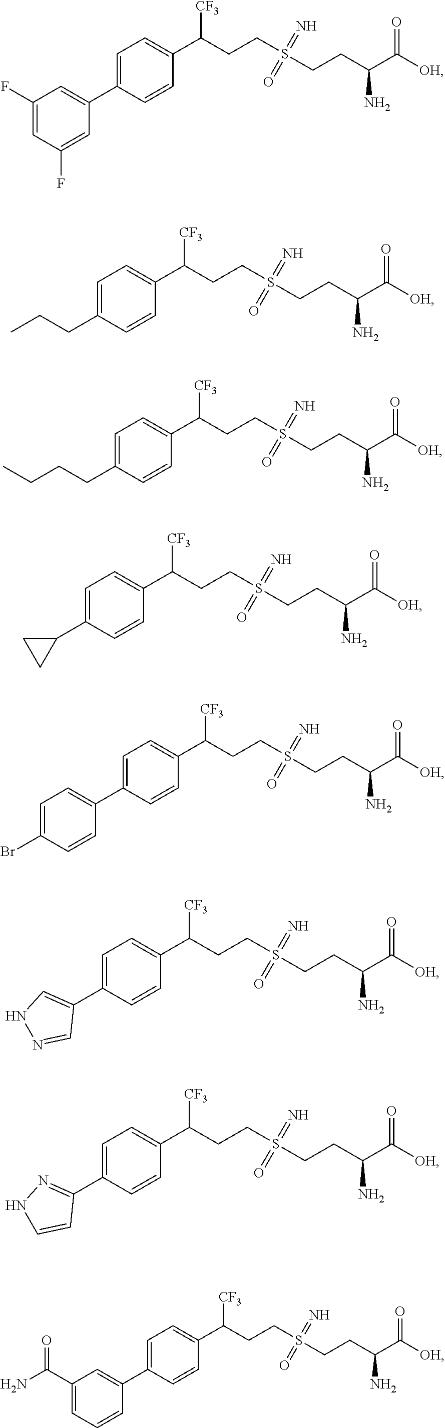

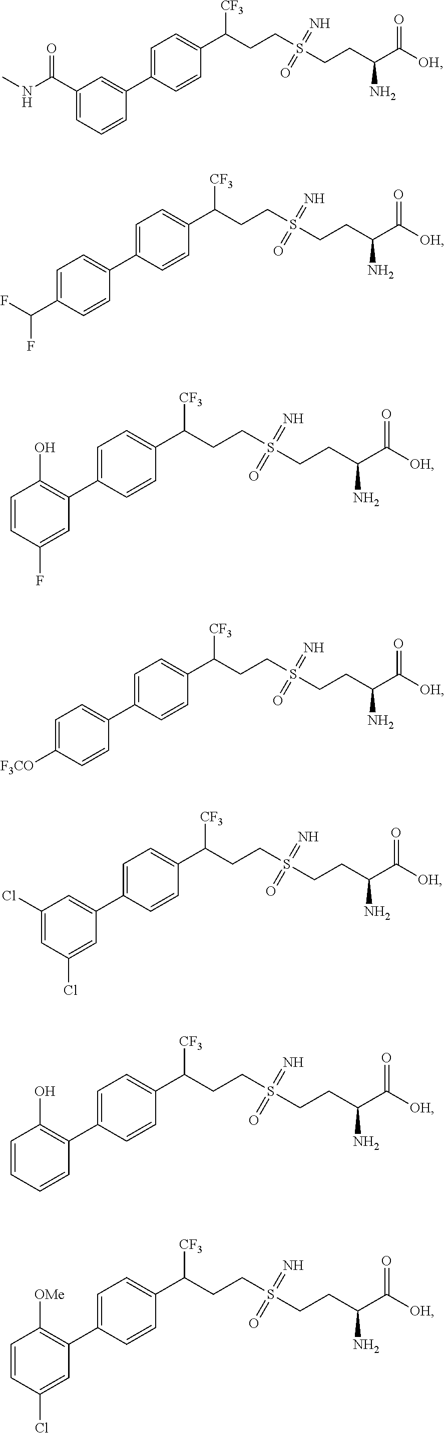

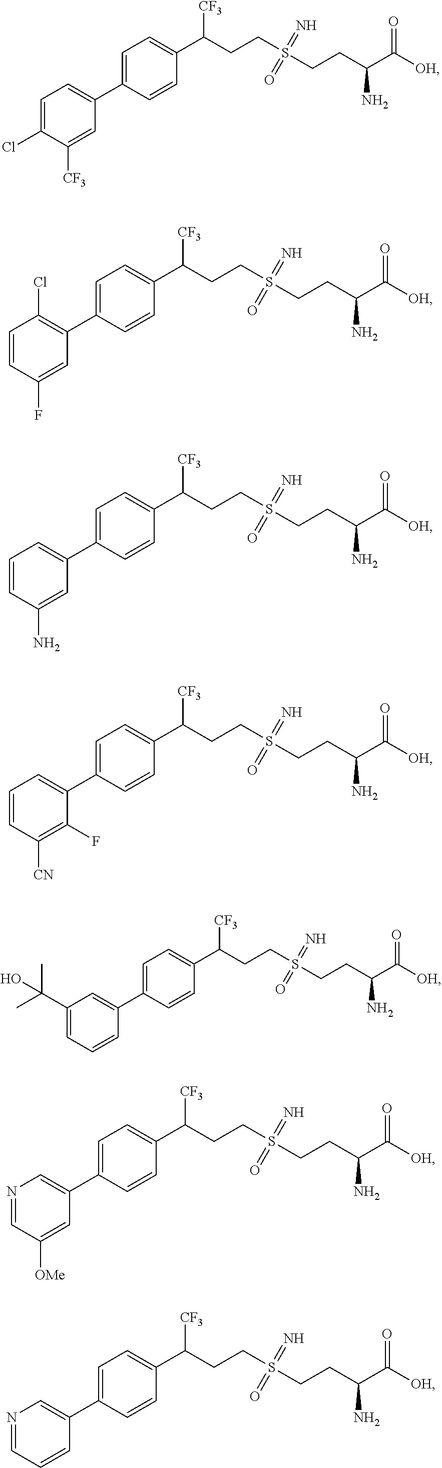

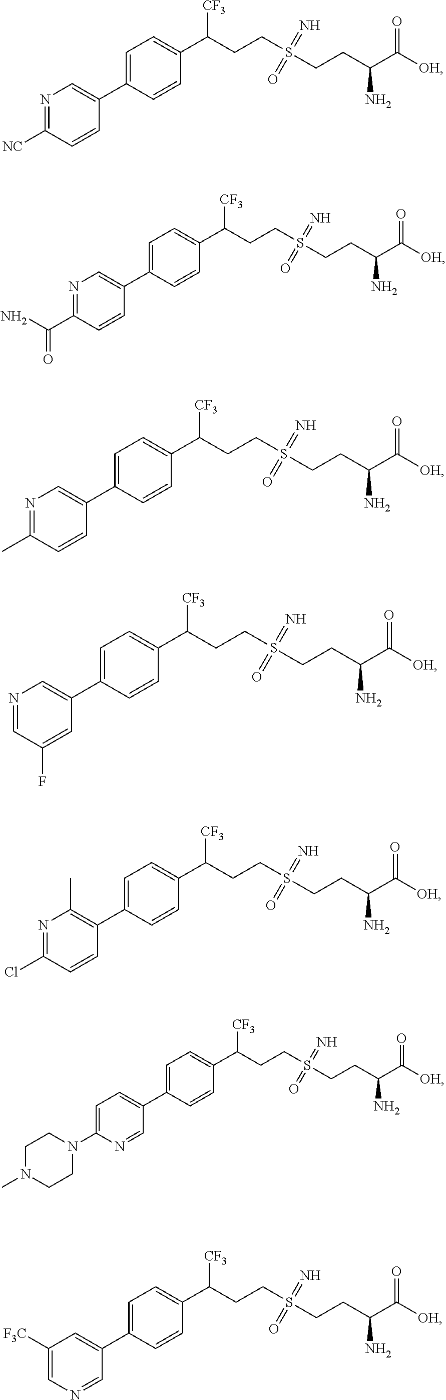

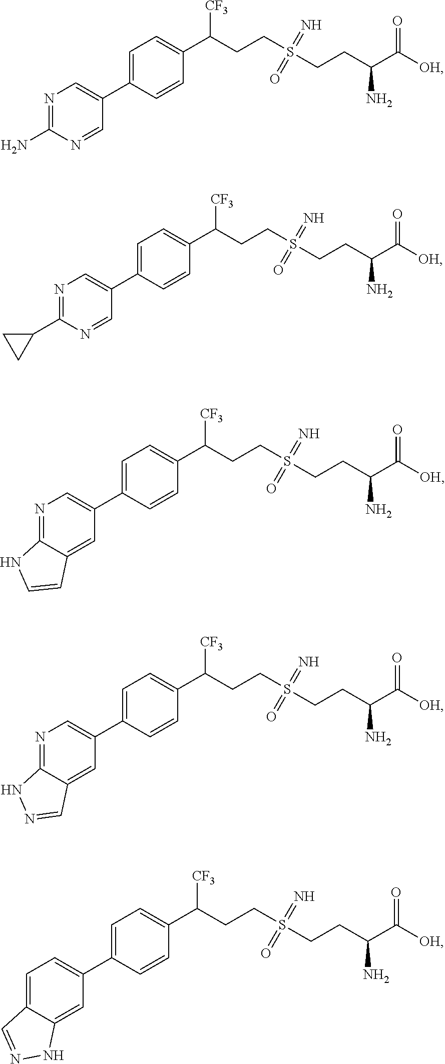 [SMILES: N=S(=O)(CCC(c1ccc(-c2cnc(N)nc2)cc1)C(F)(F)F)CC[C@H](N)C(=O)O ; N=S(=O)(CCC(c1ccc(-c2cnc(C3CC3)nc2)cc1)C(F)(F)F)CC[C@H](N)C(=O)O ; N=S(=O)(CCC(c1ccc(-c2cnc3[nH]ccc3c2)cc1)C(F)(F)F)CC[C@H](N)C(=O)O ; N=S(=O)(CCC(c1ccc(-c2cnc3[nH]ncc3c2)cc1)C(F)(F)F)CC[C@H](N)C(=O)O ; N=S(=O)(CCC(c1ccc(-c2ccc3cn[nH]c3c2)cc1)C(F)(F)F)CC[C@H](N)C(=O)O]

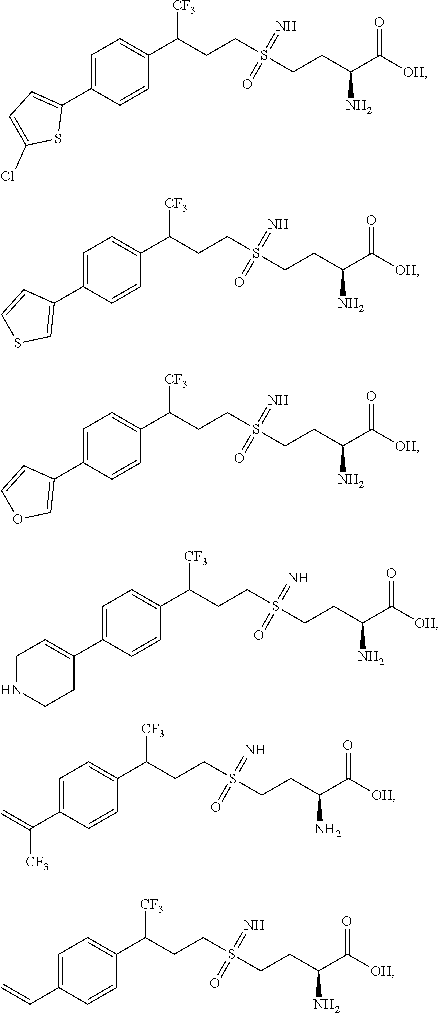 [SMILES: N=S(=O)(CCC(c1ccc(-c2ccc(Cl)s2)cc1)C(F)(F)F)CC[C@H](N)C(=O)O ; N=S(=O)(CCC(c1ccc(-c2ccsc2)cc1)C(F)(F)F)CC[C@H](N)C(=O)O ; N=S(=O)(CCC(c1ccc(-c2ccoc2)cc1)C(F)(F)F)CC[C@H](N)C(=O)O ; N=S(=O)(CCC(c1ccc(C2=CCNCC2)cc1)C(F)(F)F)CC[C@H](N)C(=O)O ; C=C(c1ccc(C(CCS(=N)(=O)CC[C@H](N)C(=O)O)C(F)(F)F)cc1)C(F)(F)F ; C=Cc1ccc(C(CCS(=N)(=O)CC[C@H](N)C(=O)O)C(F)(F)F)cc1]

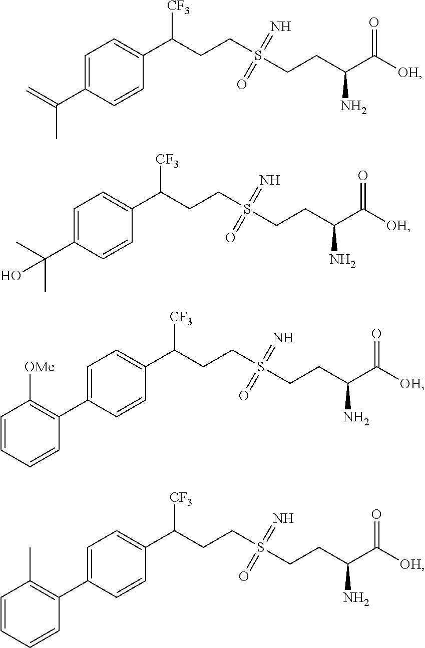 [SMILES: C=C(C)c1ccc(C(CCS(=N)(=O)CC[C@H](N)C(=O)O)C(F)(F)F)cc1 ; CC(C)(O)c1ccc(C(CCS(=N)(=O)CC[C@H](N)C(=O)O)C(F)(F)F)cc1 ; COc1ccccc1-c1ccc(C(CCS(=N)(=O)CC[C@H](N)C(=O)O)C(F)(F)F)cc1 ; Cc1ccccc1-c1ccc(C(CCS(=N)(=O)CC[C@H](N)C(=O)O)C(F)(F)F)cc1]

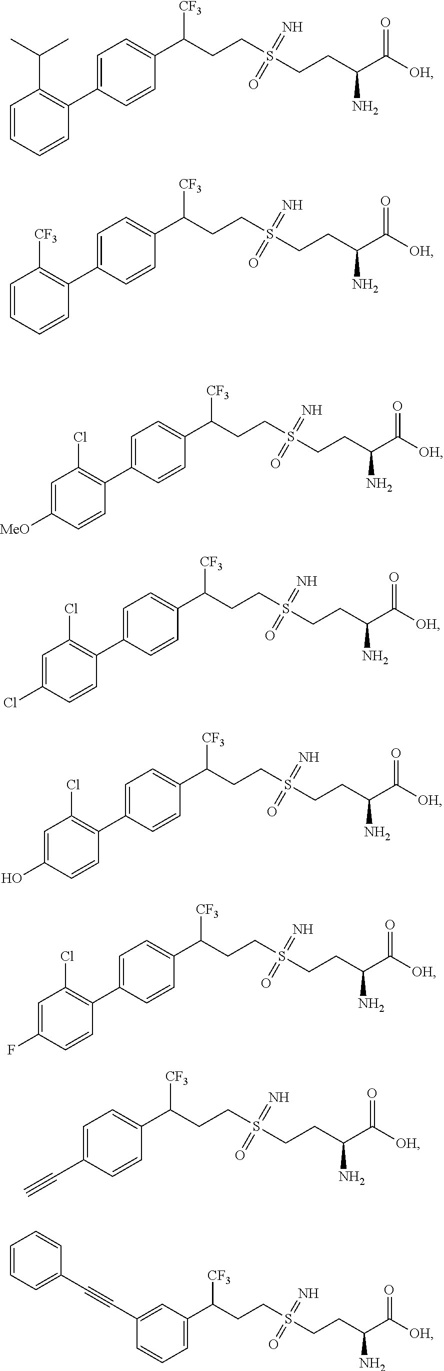

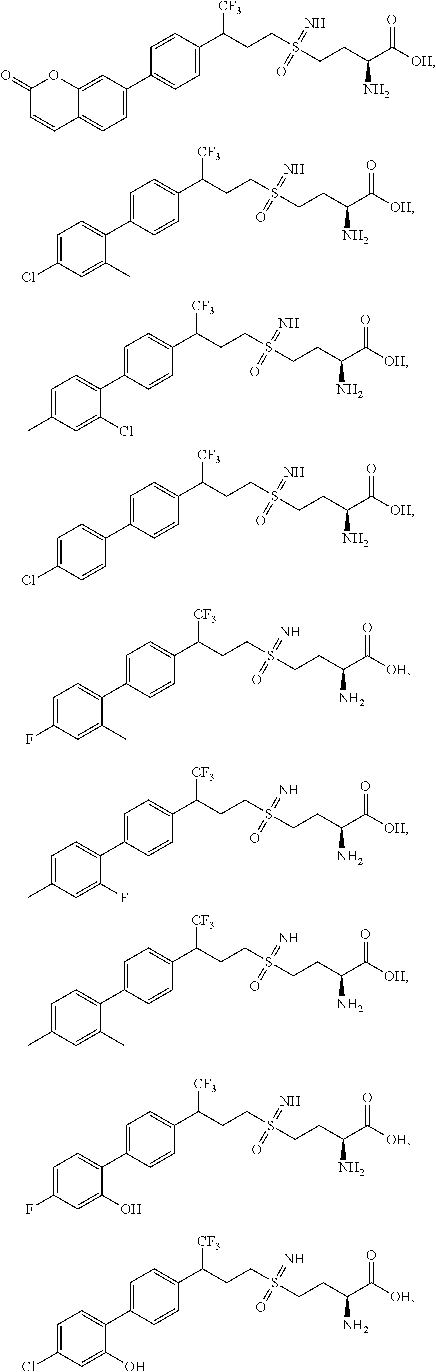

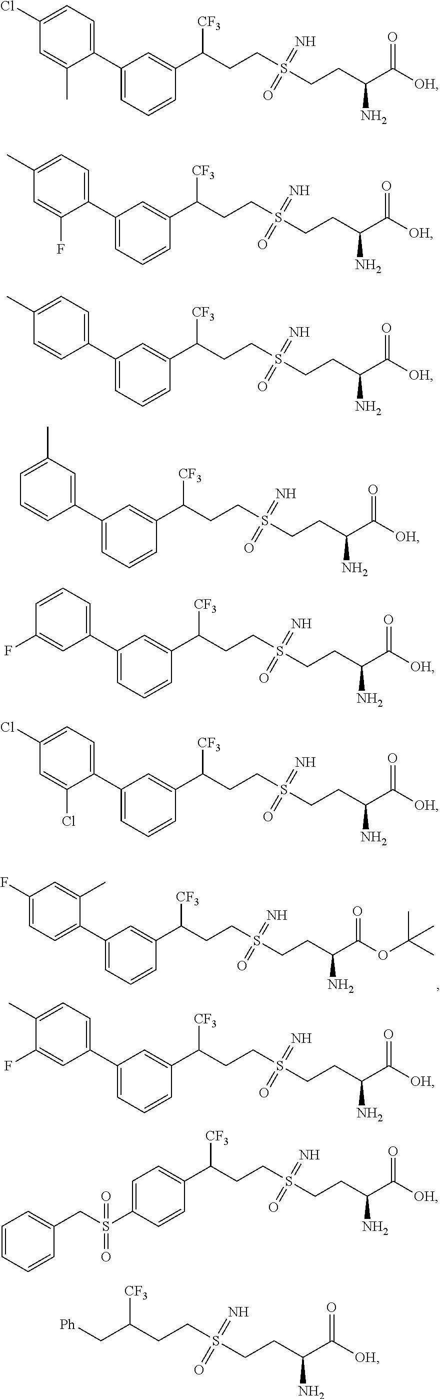

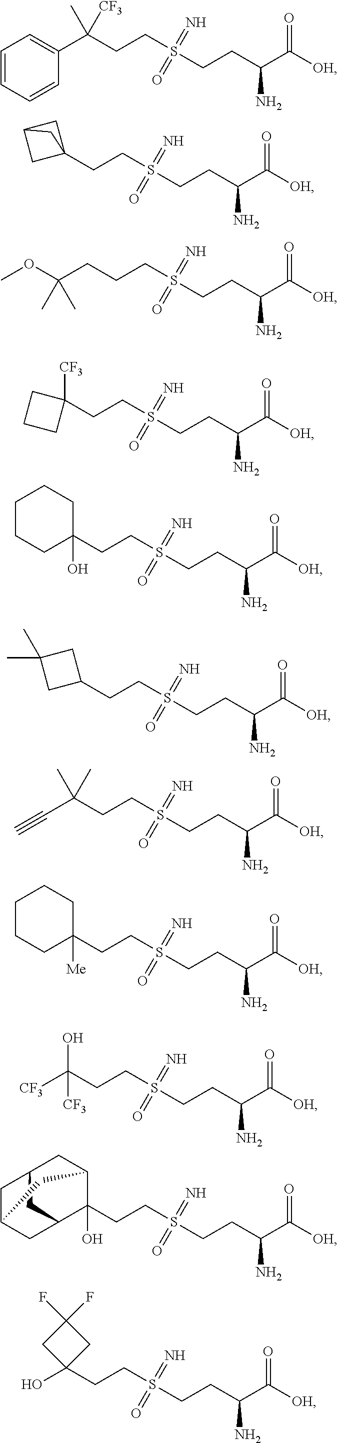

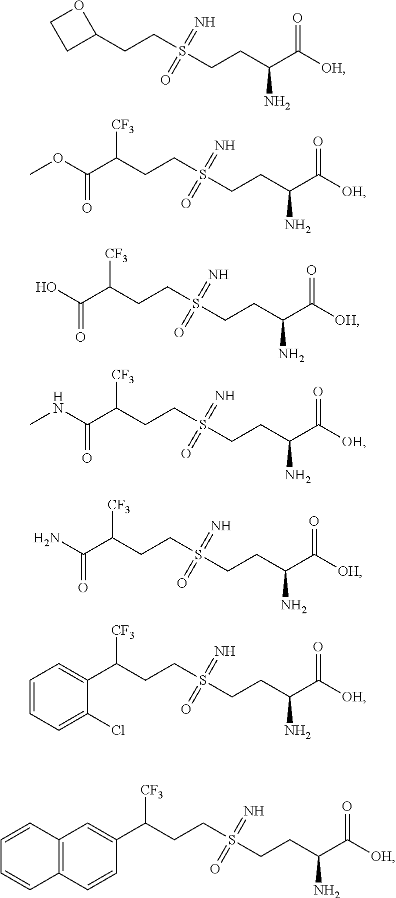

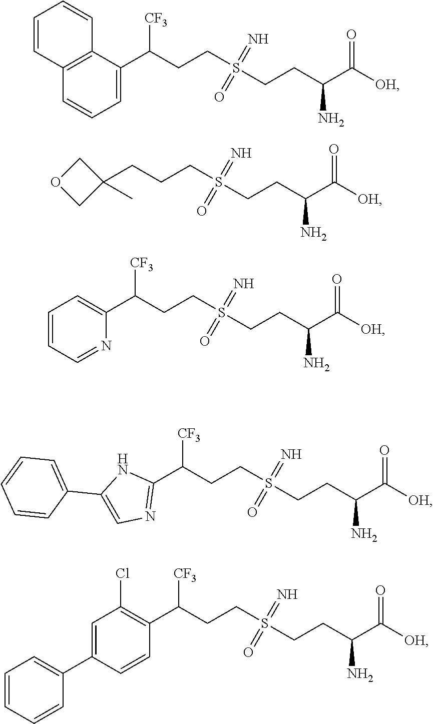

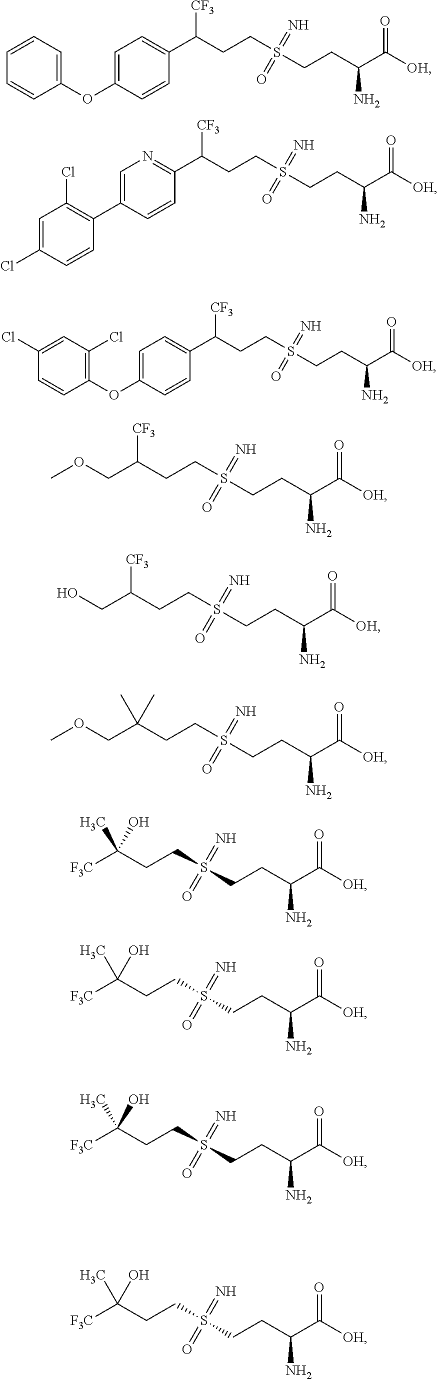

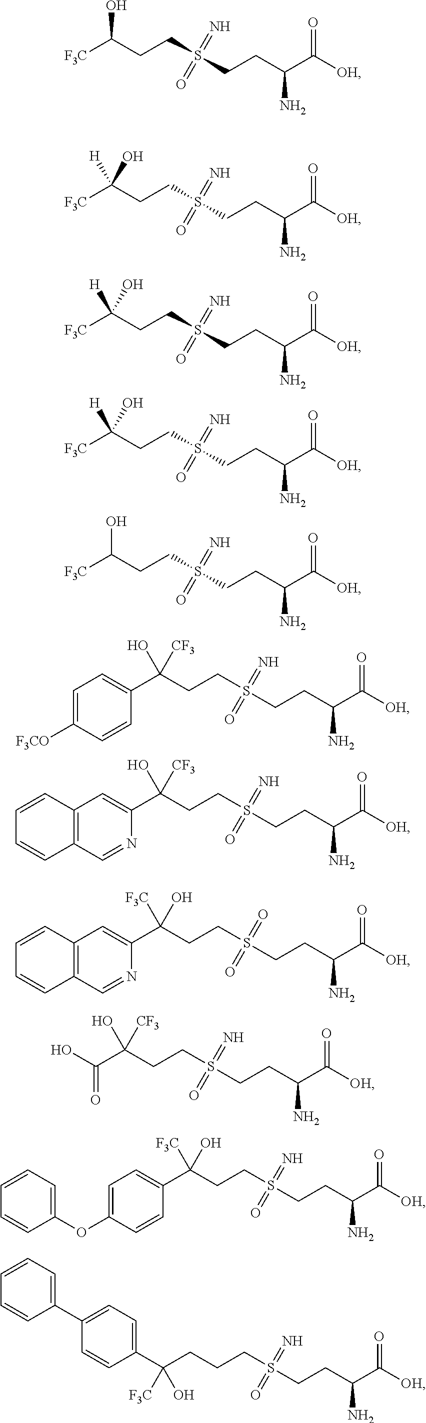

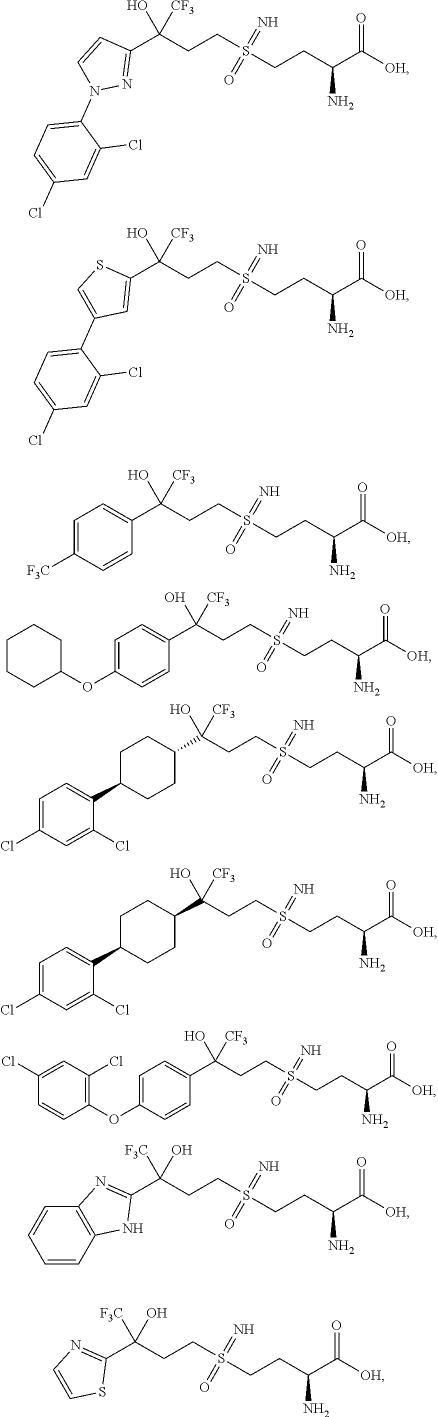

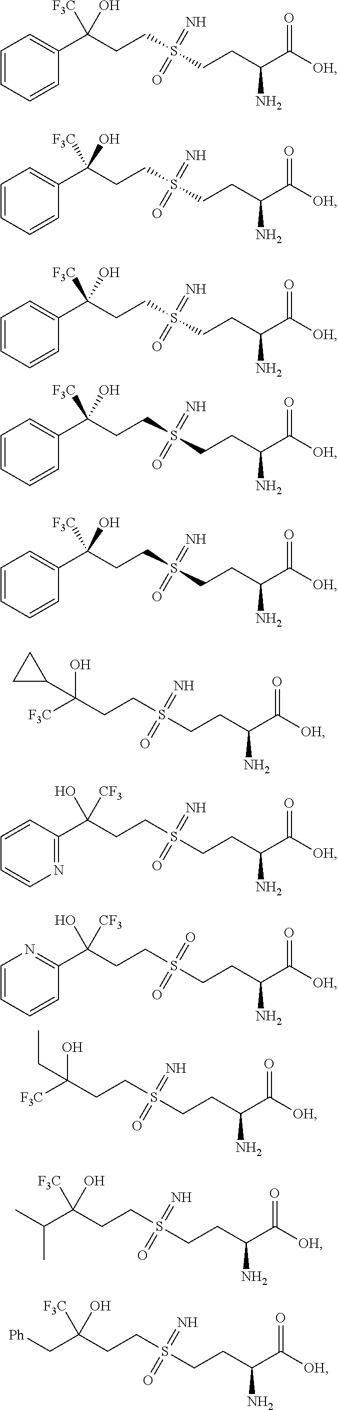

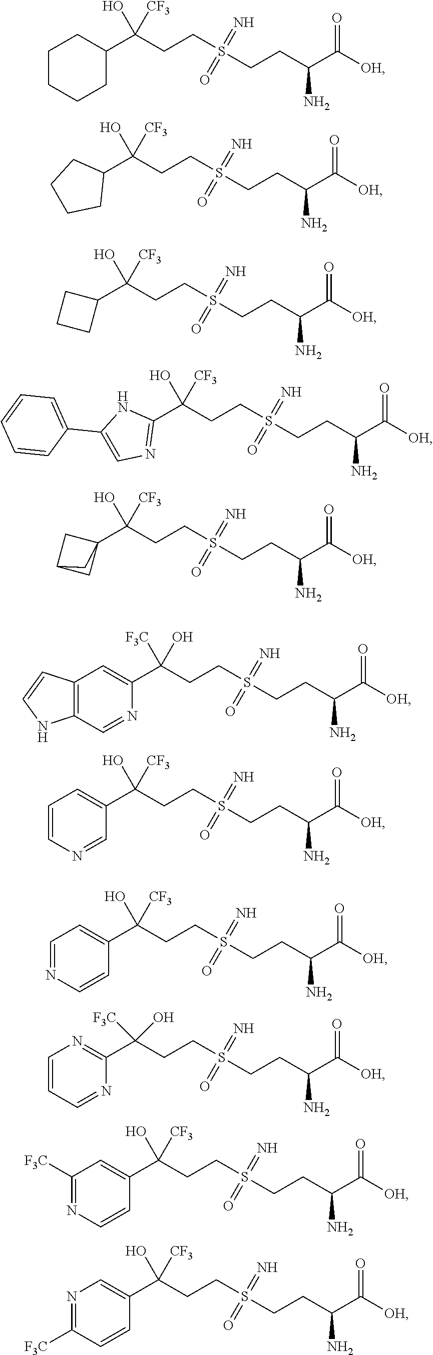

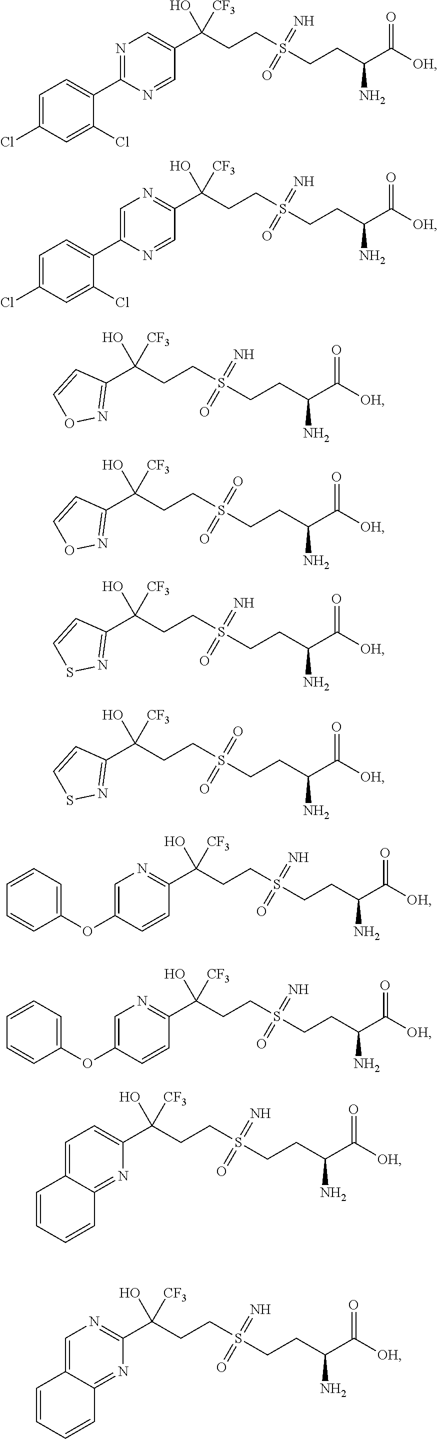

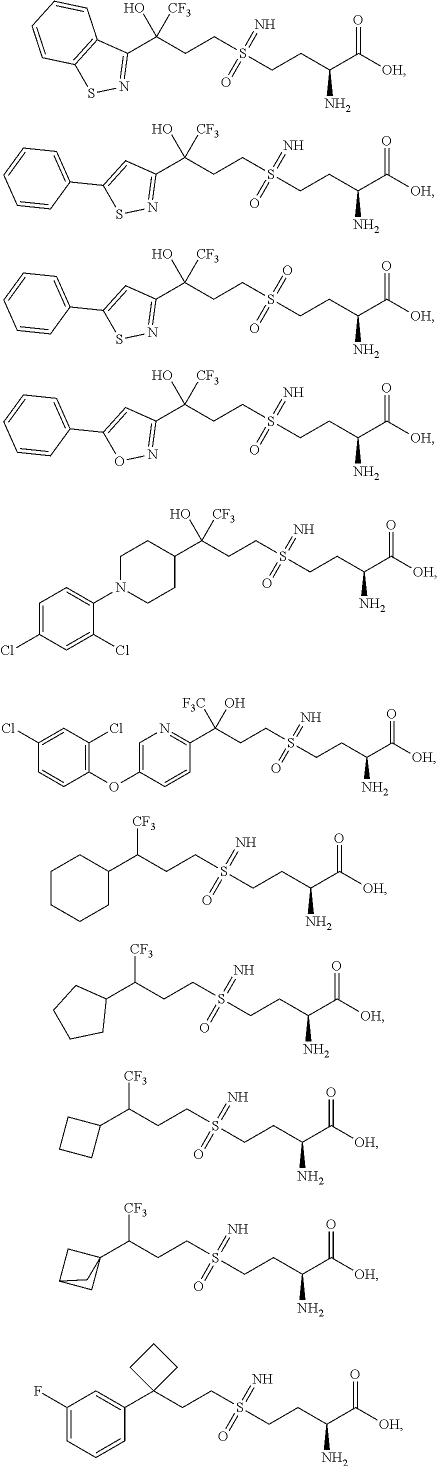

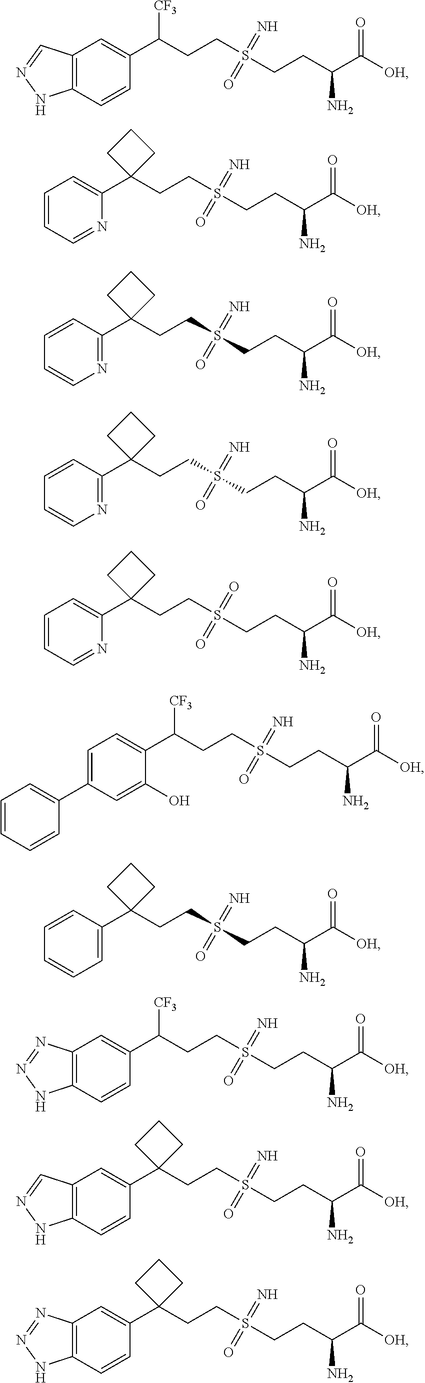

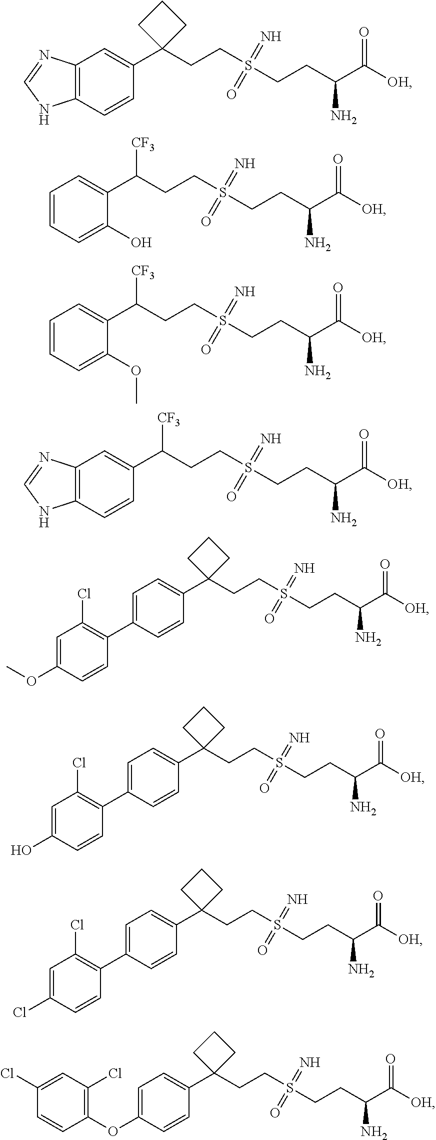

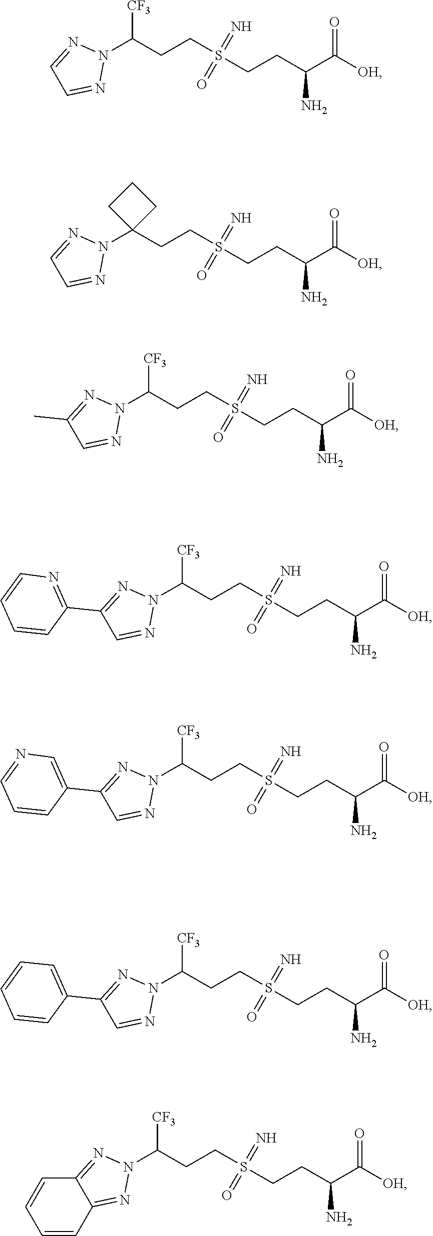

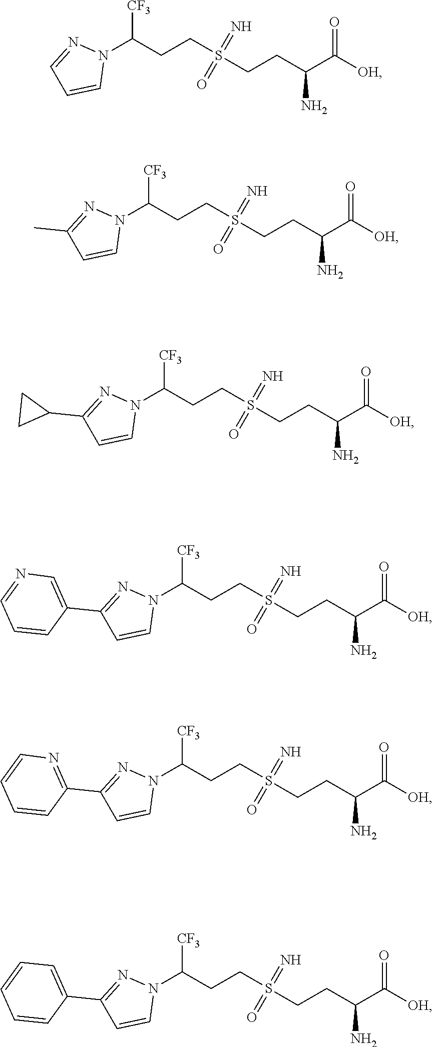 [SMILES: N=S(=O)(CCC(n1cccn1)C(F)(F)F)CC[C@H](N)C(=O)O ; Cc1ccn(C(CCS(=N)(=O)CC[C@H](N)C(=O)O)C(F)(F)F)n1 ; N=S(=O)(CCC(n1ccc(C2CC2)n1)C(F)(F)F)CC[C@H](N)C(=O)O ; N=S(=O)(CCC(n1ccc(-c2cccnc2)n1)C(F)(F)F)CC[C@H](N)C(=O)O ; N=S(=O)(CCC(n1ccc(-c2ccccn2)n1)C(F)(F)F)CC[C@H](N)C(=O)O ; N=S(=O)(CCC(n1ccc(-c2ccccc2)n1)C(F)(F)F)CC[C@H](N)C(=O)O]

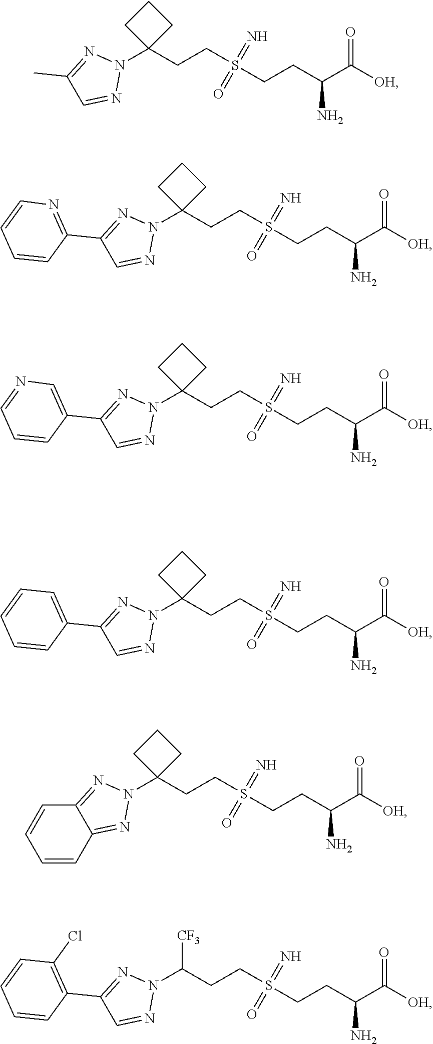 [SMILES: Cc1cnn(C2(CCS(=N)(=O)CC[C@H](N)C(=O)O)CCC2)n1 ; N=S(=O)(CC[C@H](N)C(=O)O)CCC1(n2ncc(-c3ccccn3)n2)CCC1 ; N=S(=O)(CC[C@H](N)C(=O)O)CCC1(n2ncc(-c3cccnc3)n2)CCC1 ; N=S(=O)(CC[C@H](N)C(=O)O)CCC1(n2ncc(-c3ccccc3)n2)CCC1 ; N=S(=O)(CC[C@H](N)C(=O)O)CCC1(n2nc3ccccc3n2)CCC1 ; N=S(=O)(CCC(n1ncc(-c2ccccc2Cl)n1)C(F)(F)F)CC[C@H](N)C(=O)O]

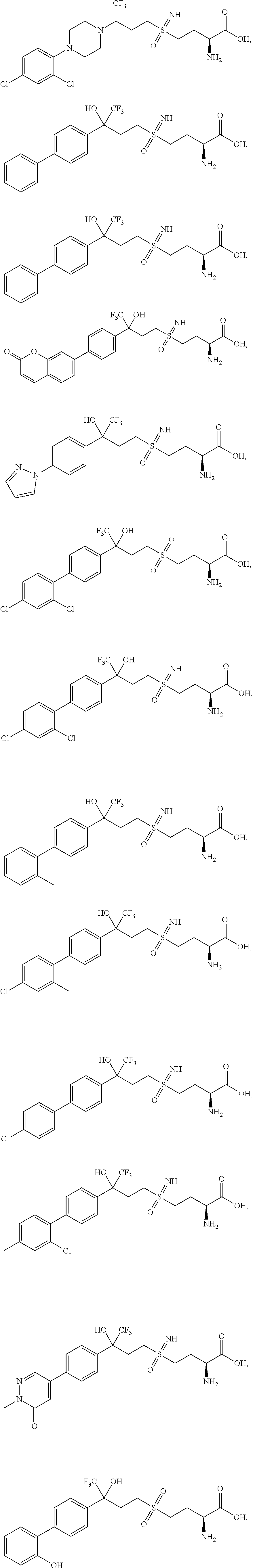

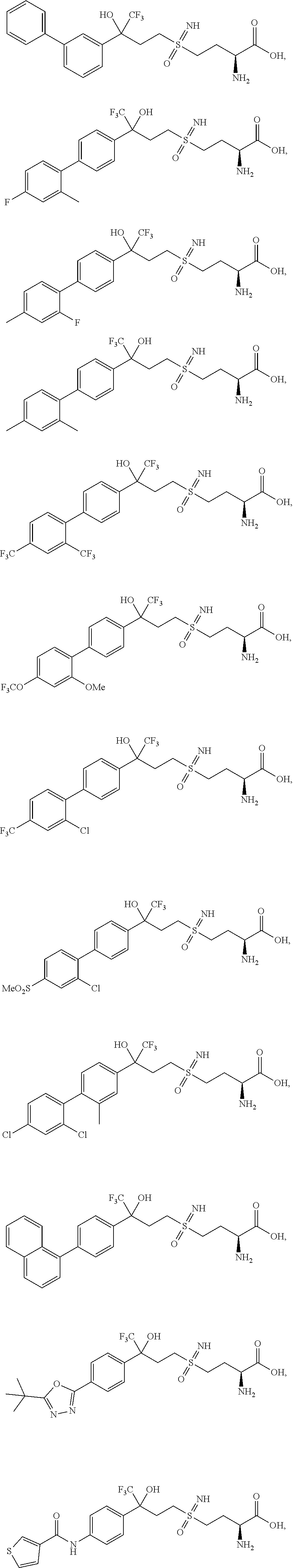

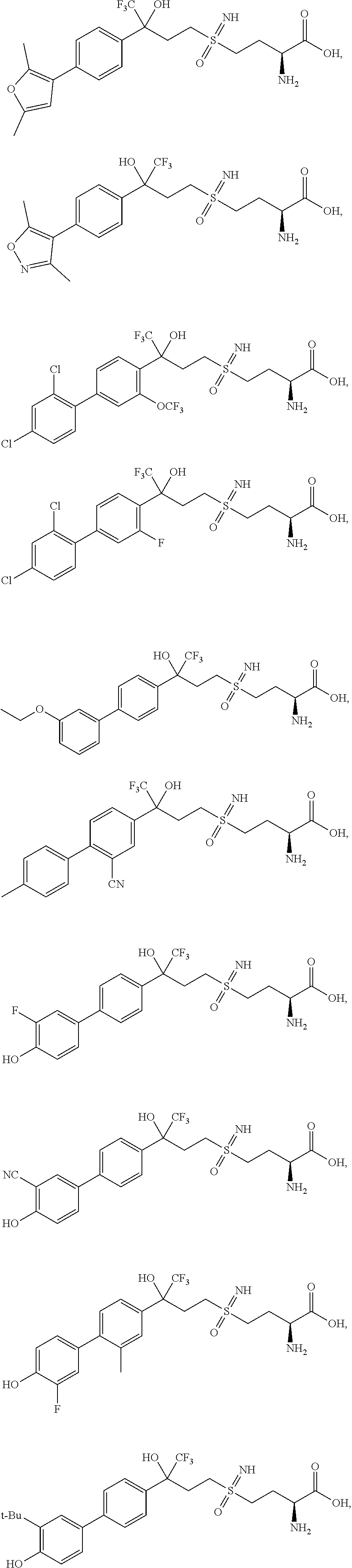

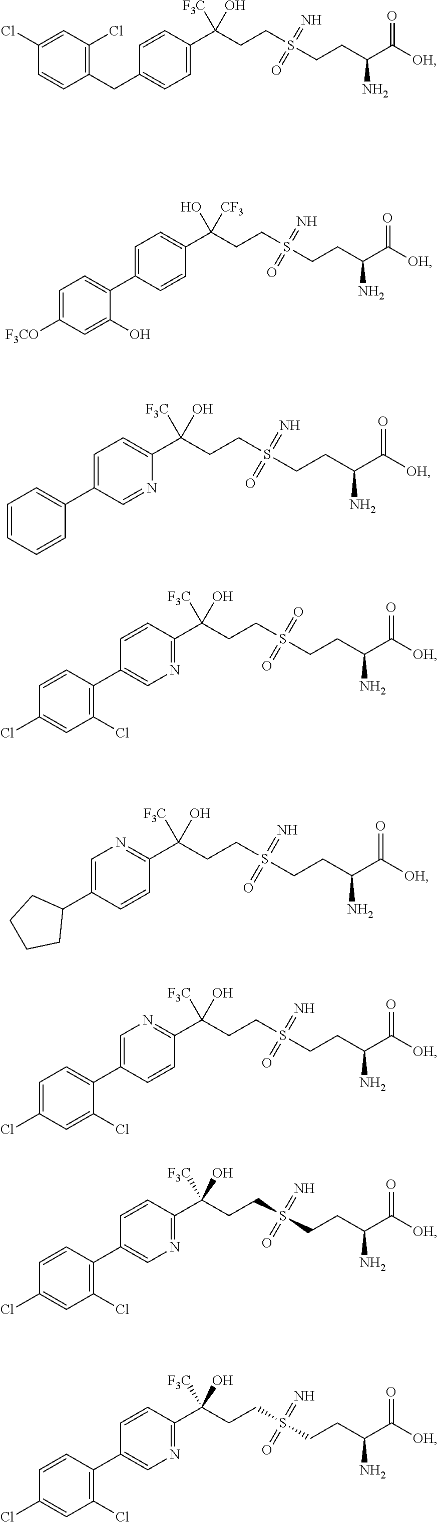

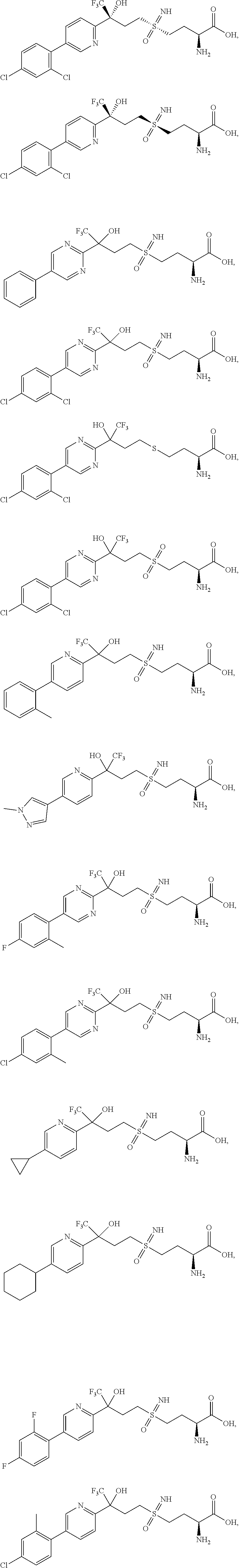

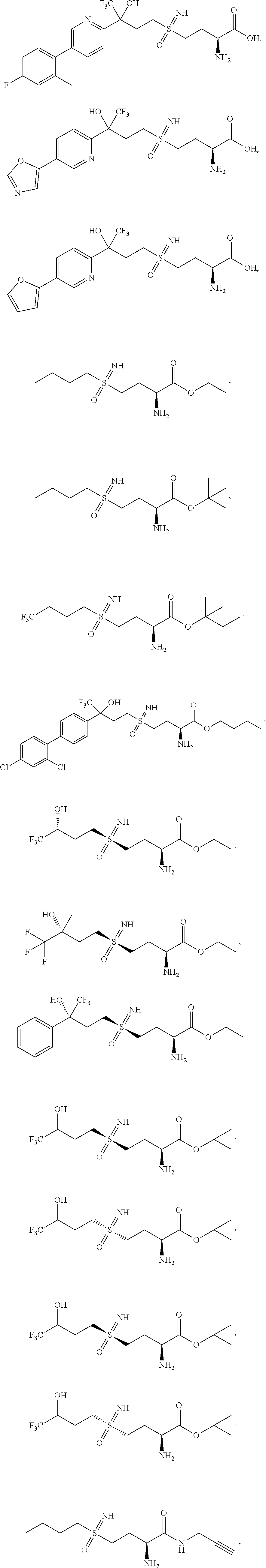

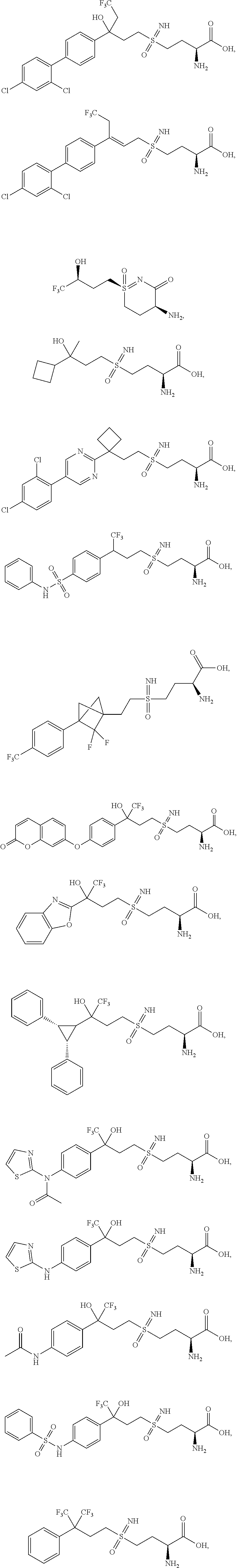

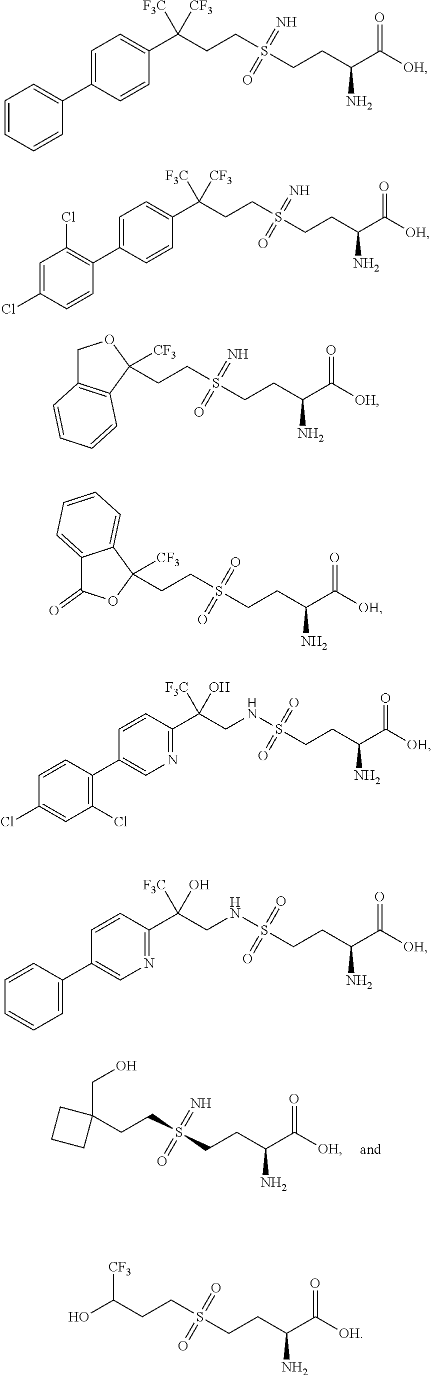

In some embodiments, is a compound, a diastereomer or an enantiomer of the compound, or a pharmaceutically acceptable salt of any of the foregoing, or a deuterated derivative of any of the foregoing; wherein the compound is selected from the group consisting of:

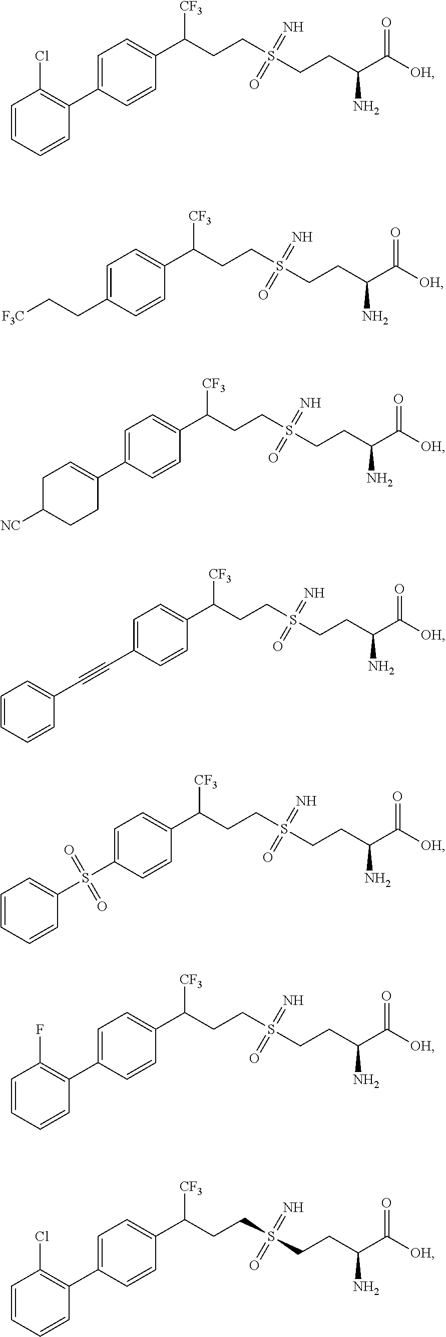

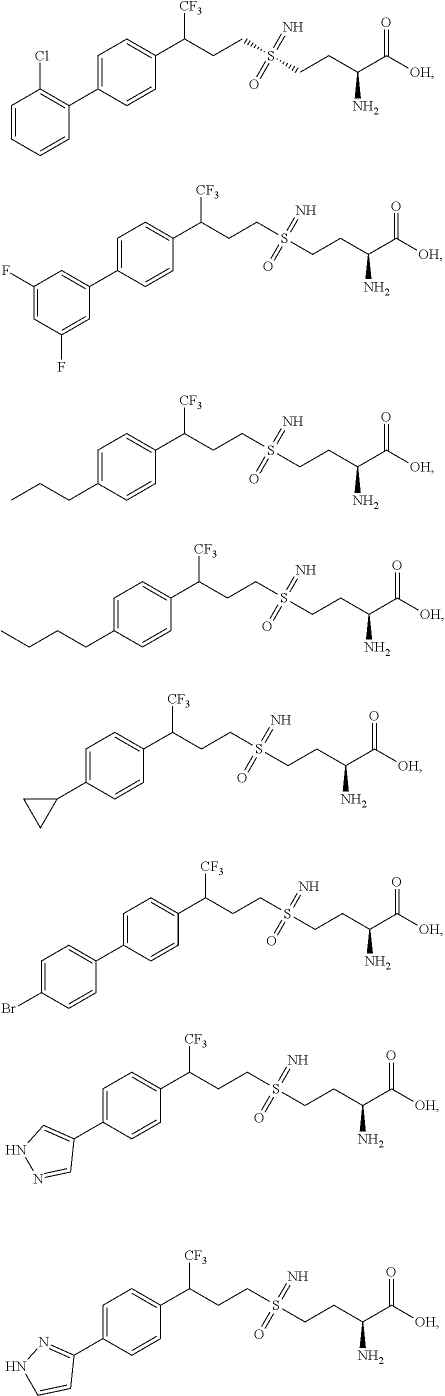

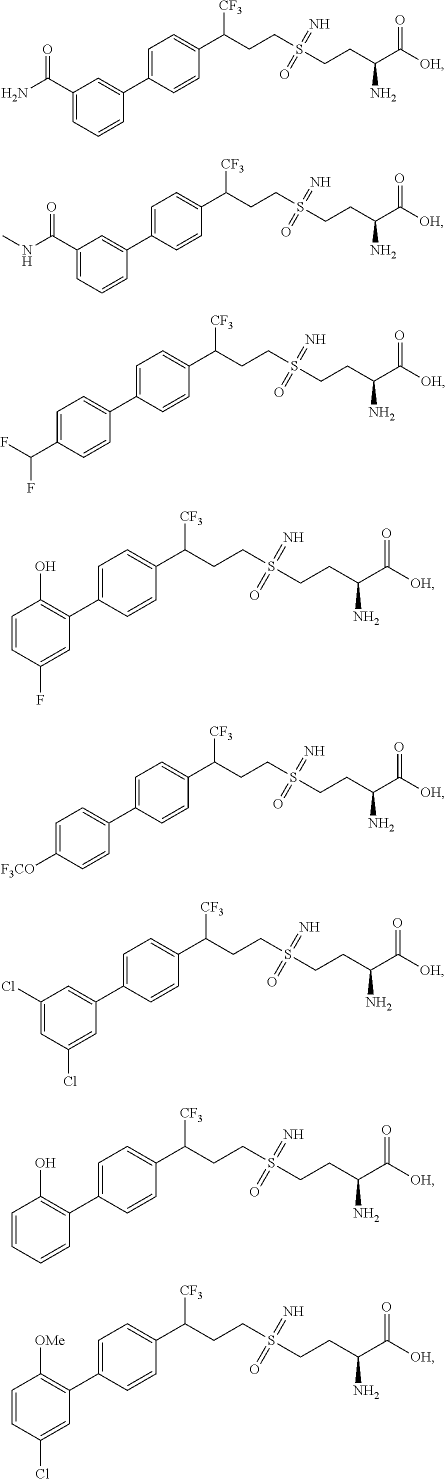

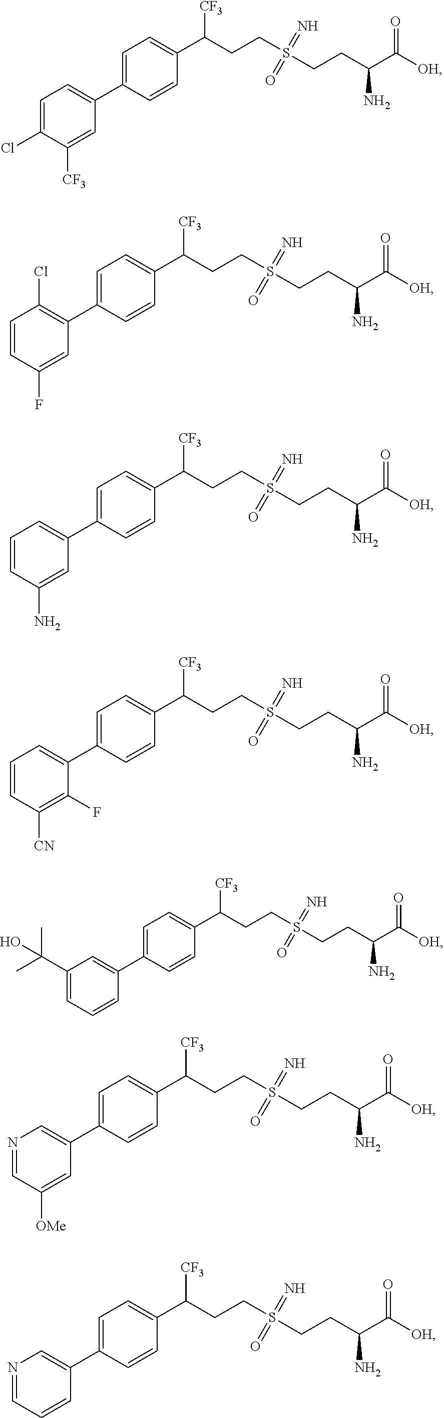

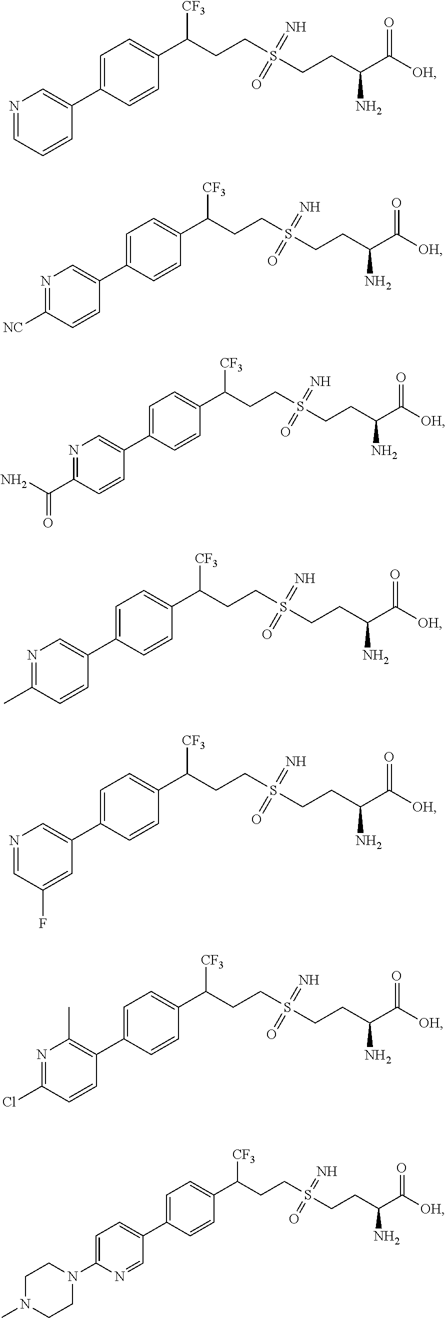

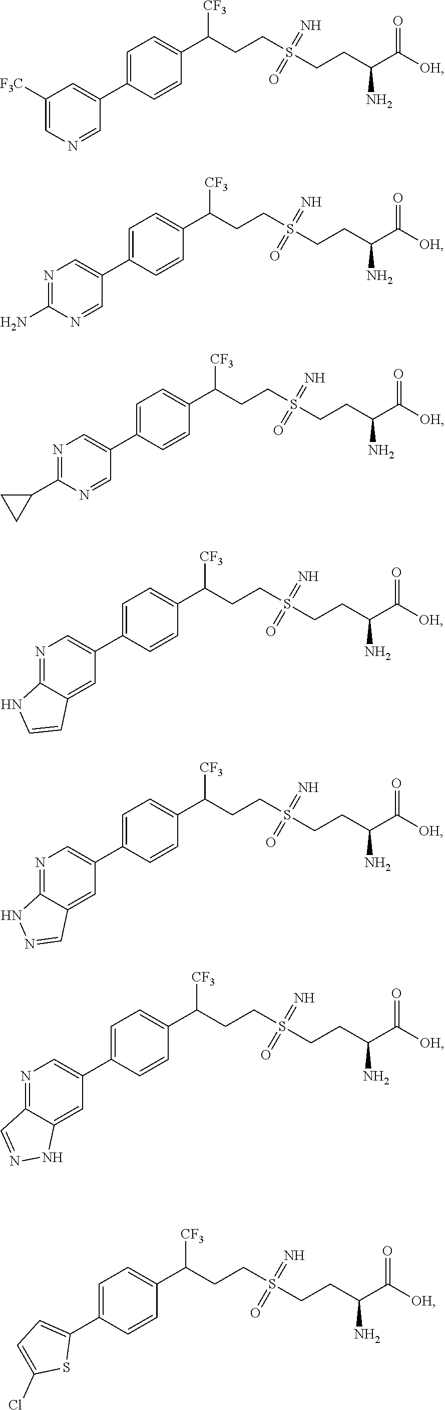

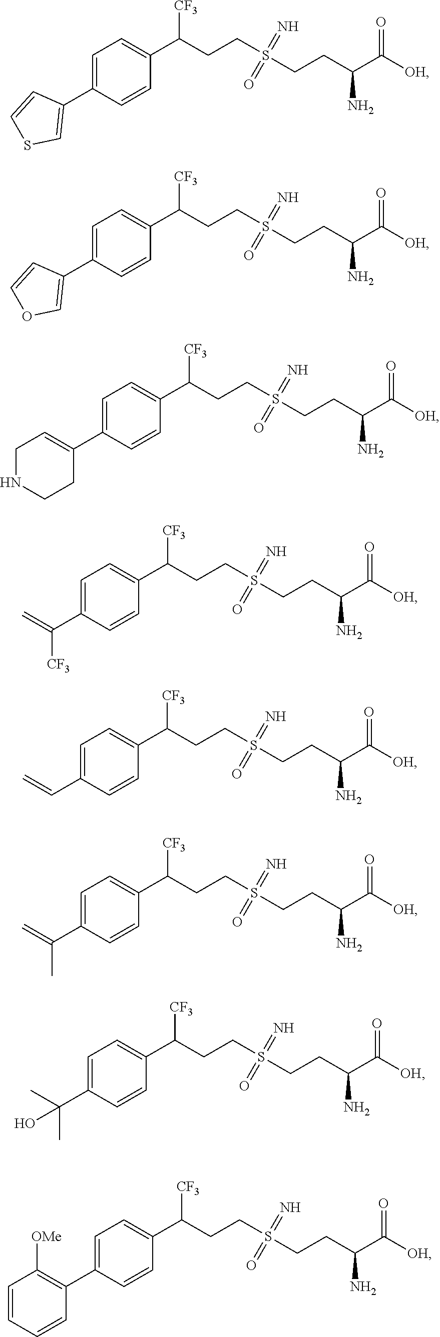

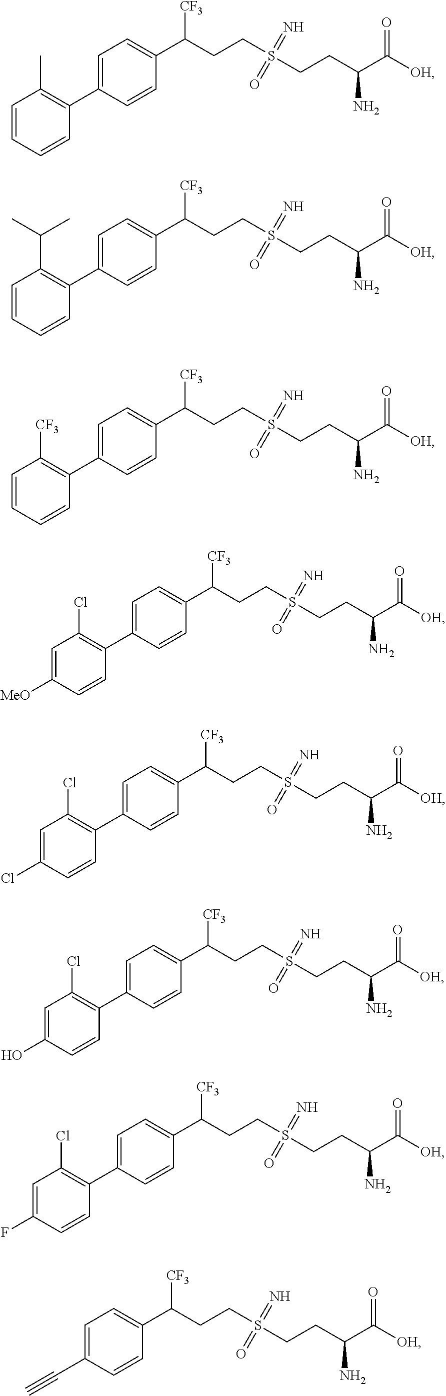

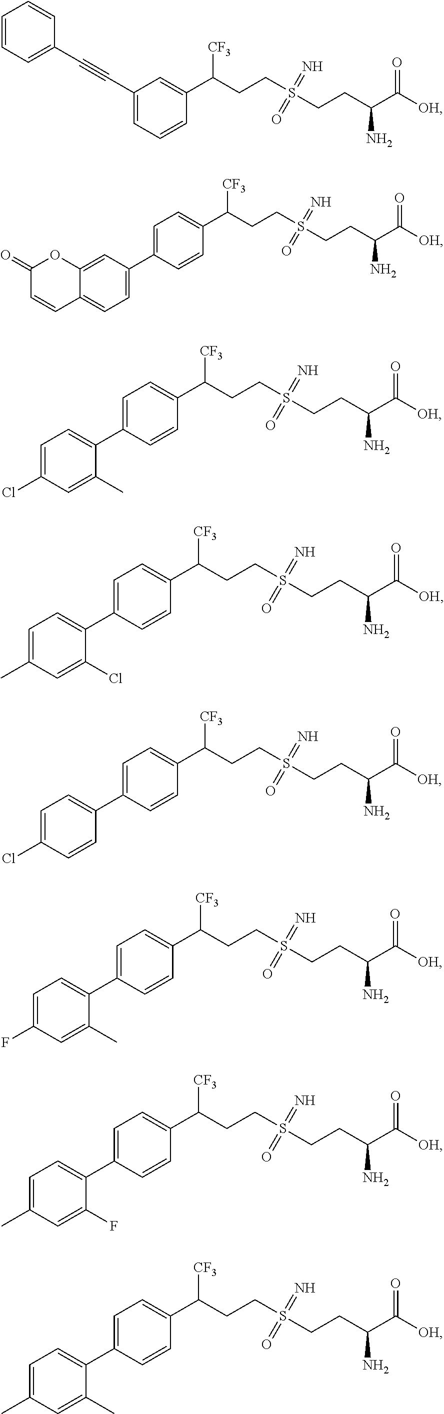

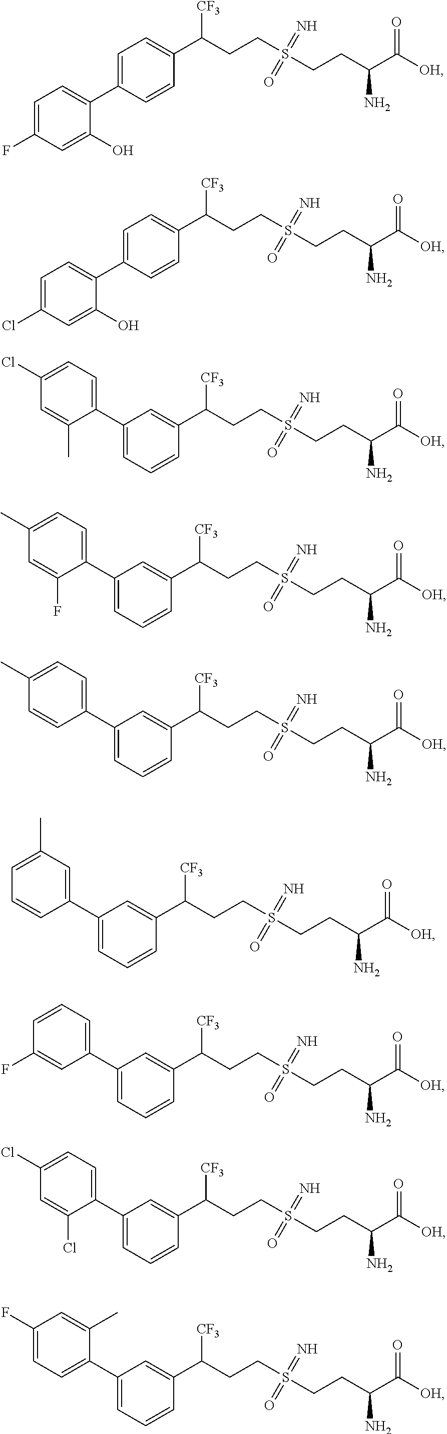

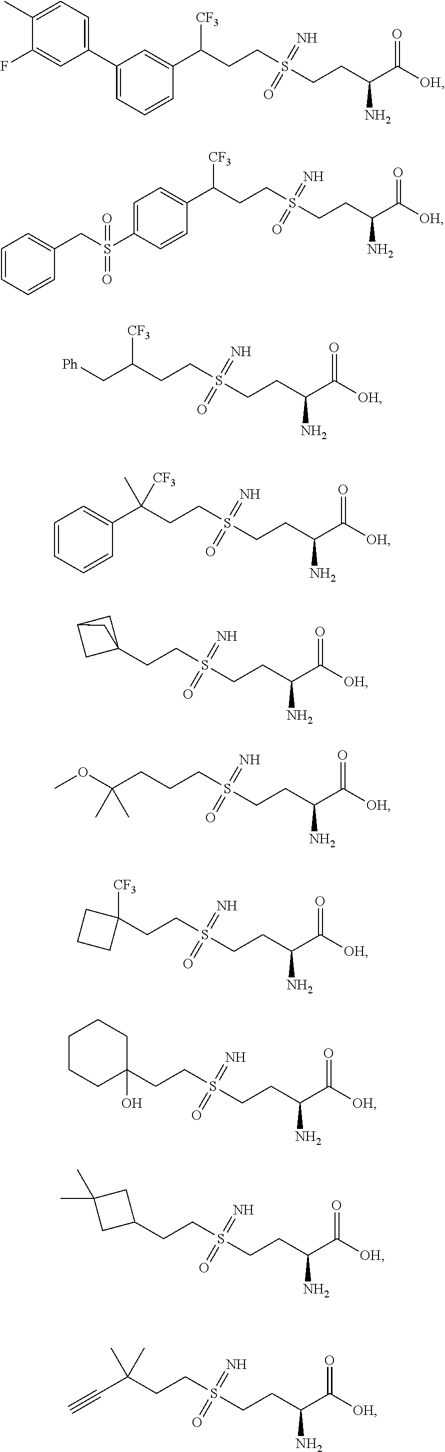

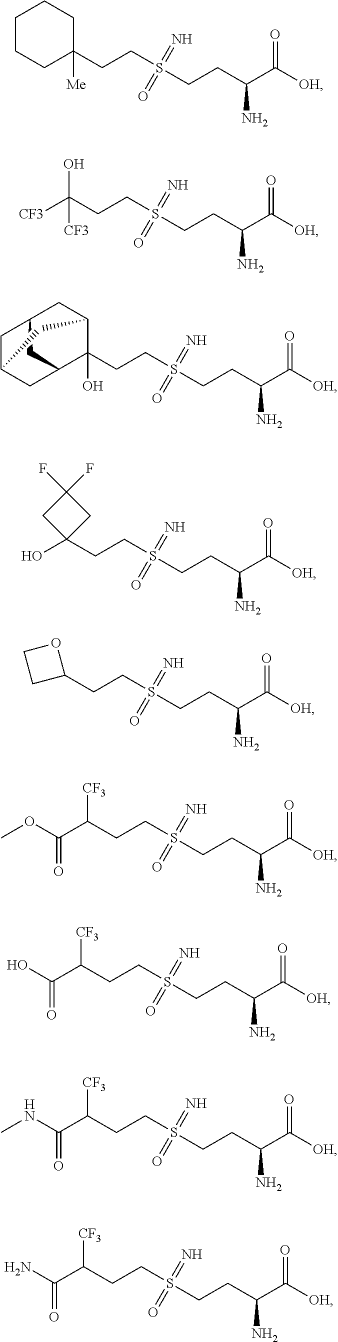

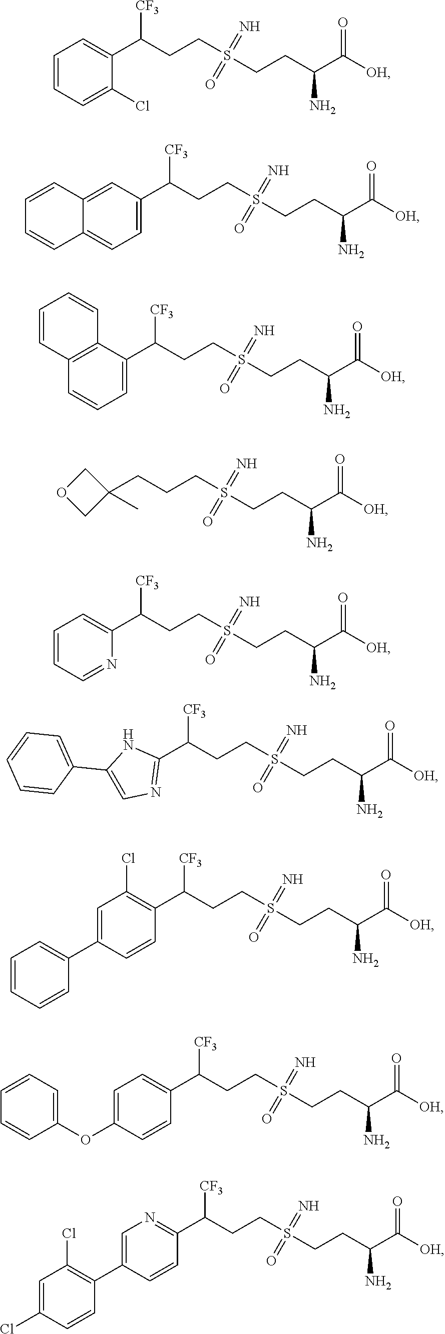

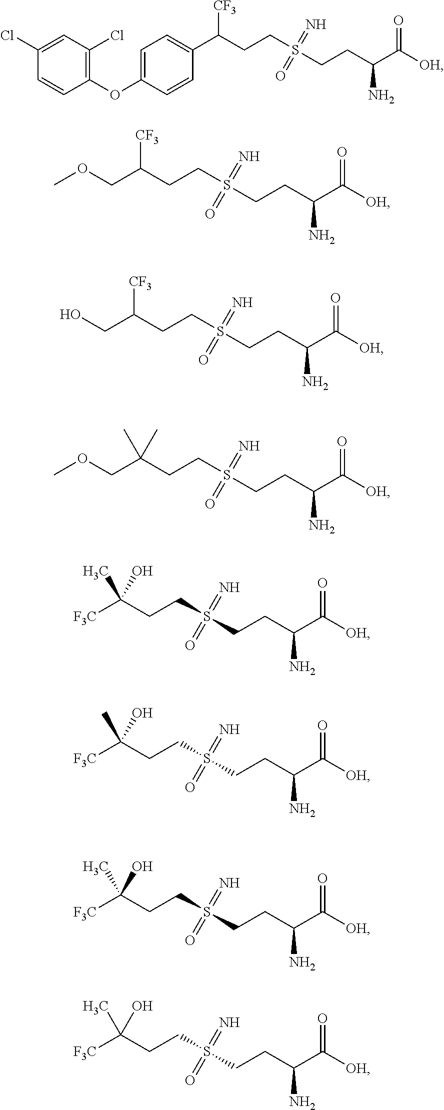

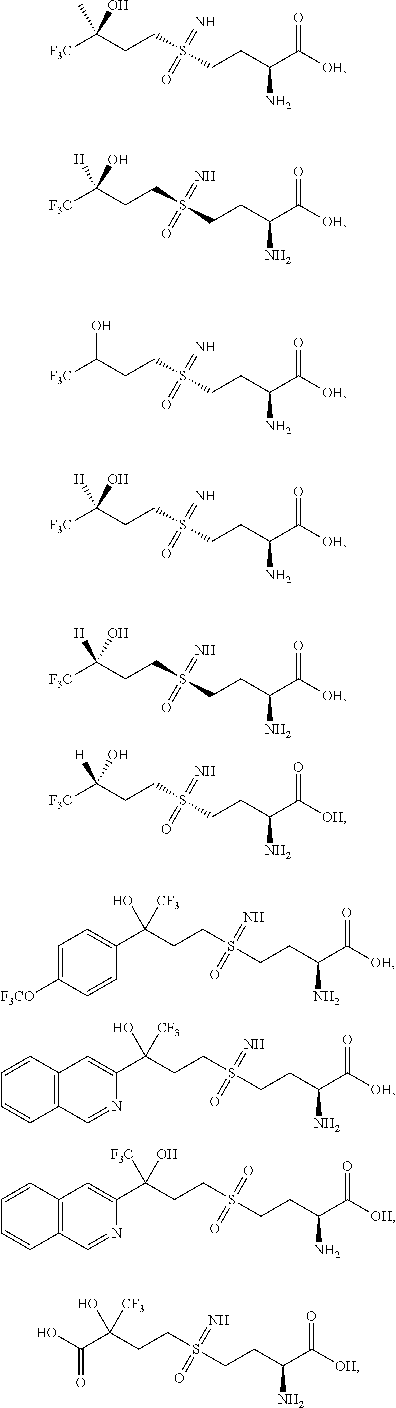

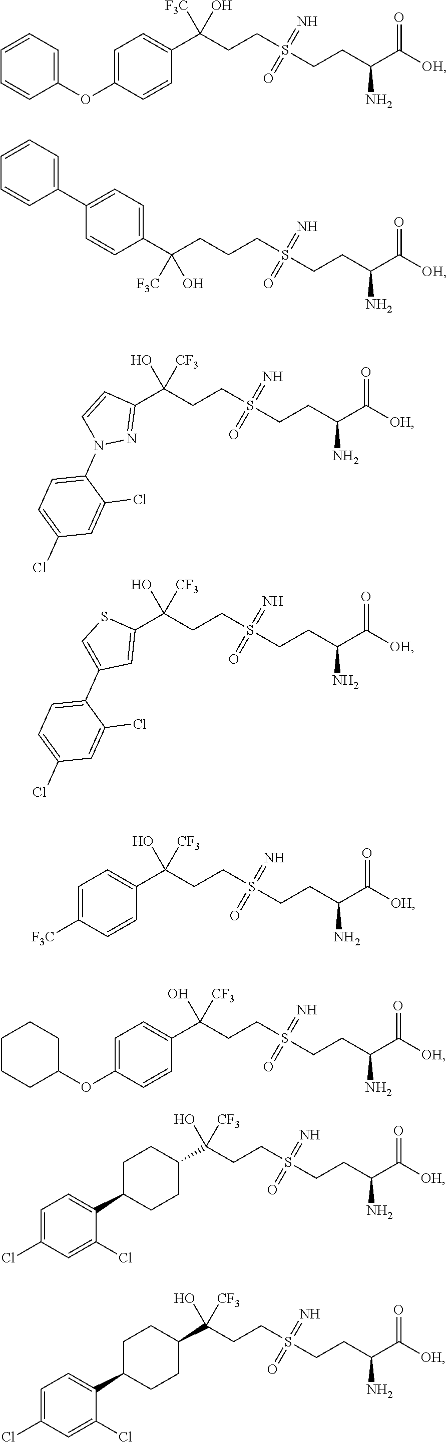

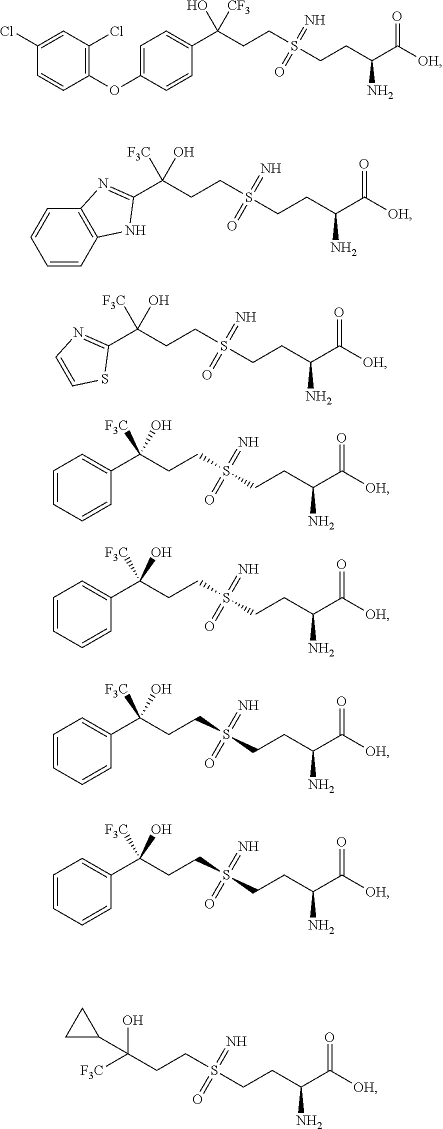

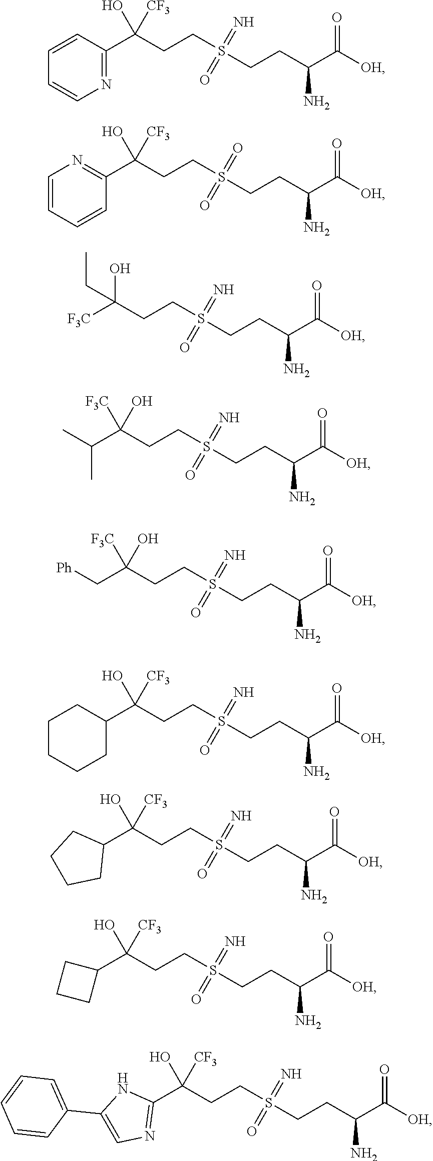

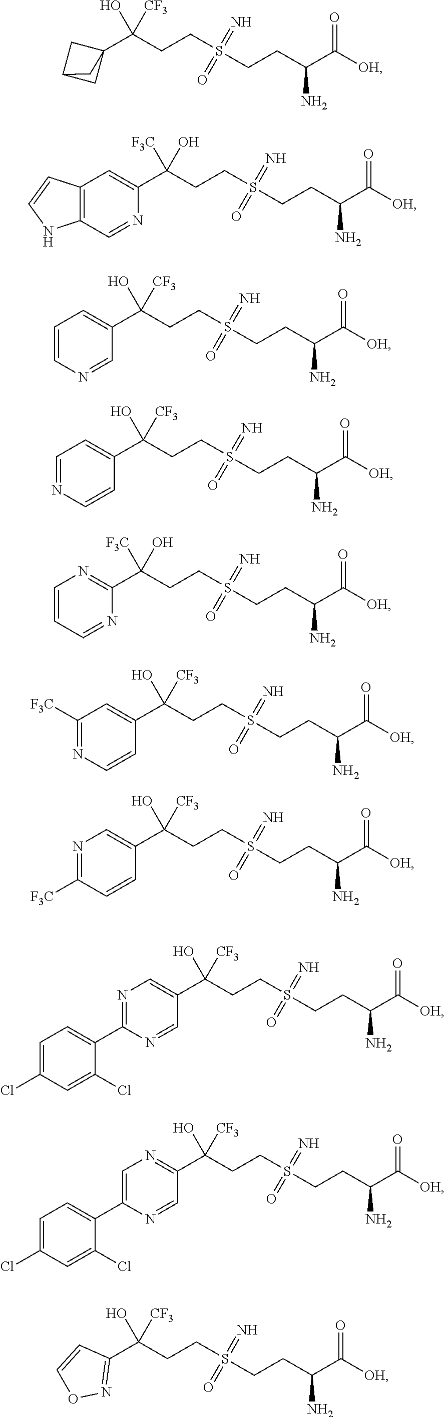

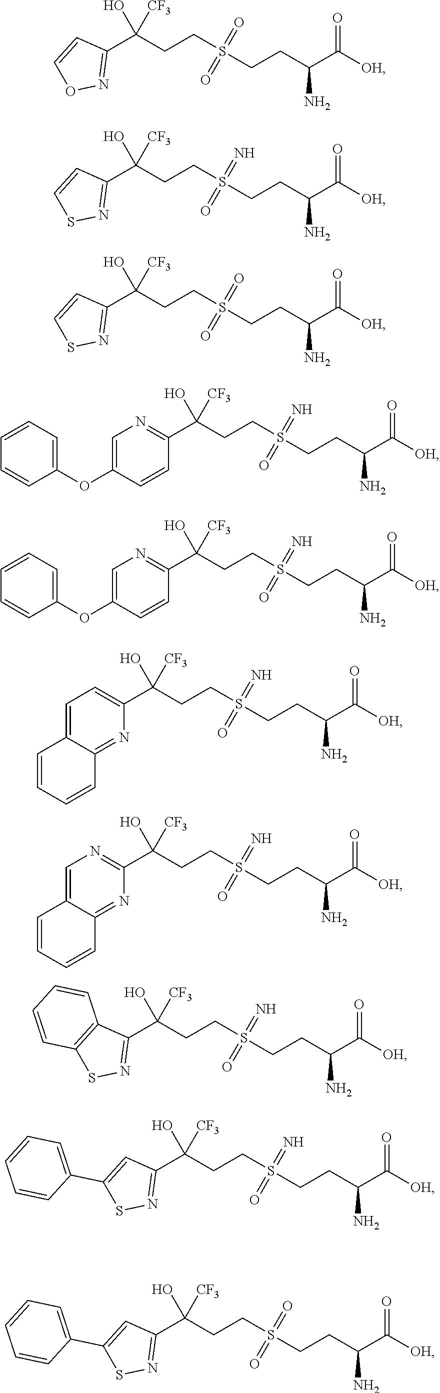

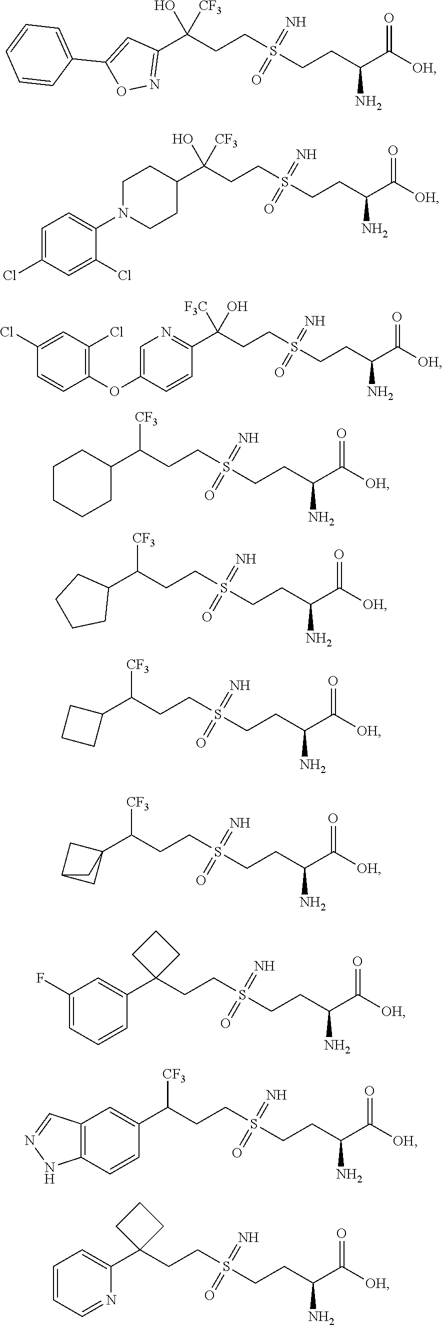

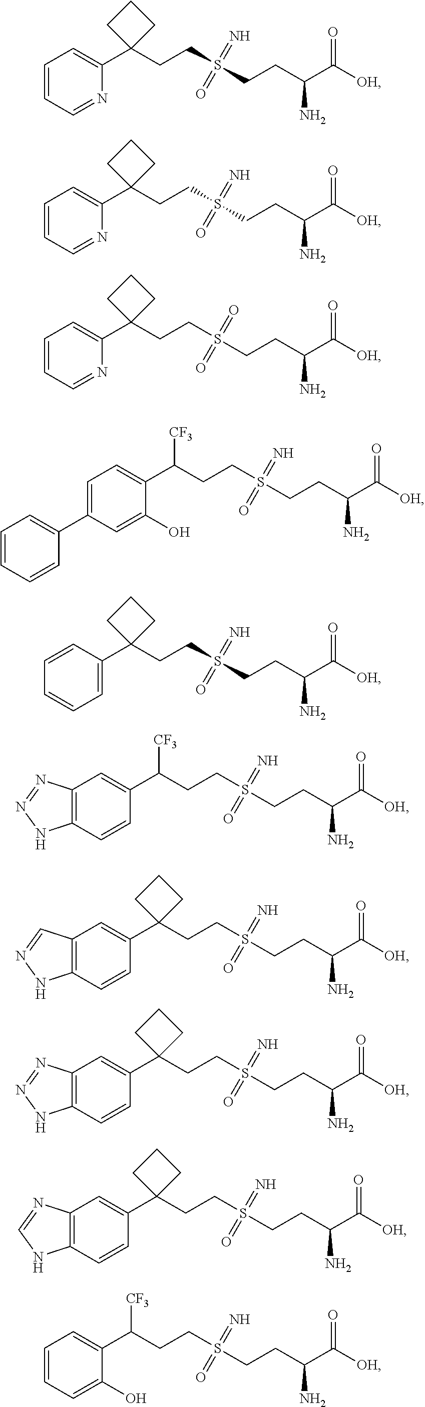

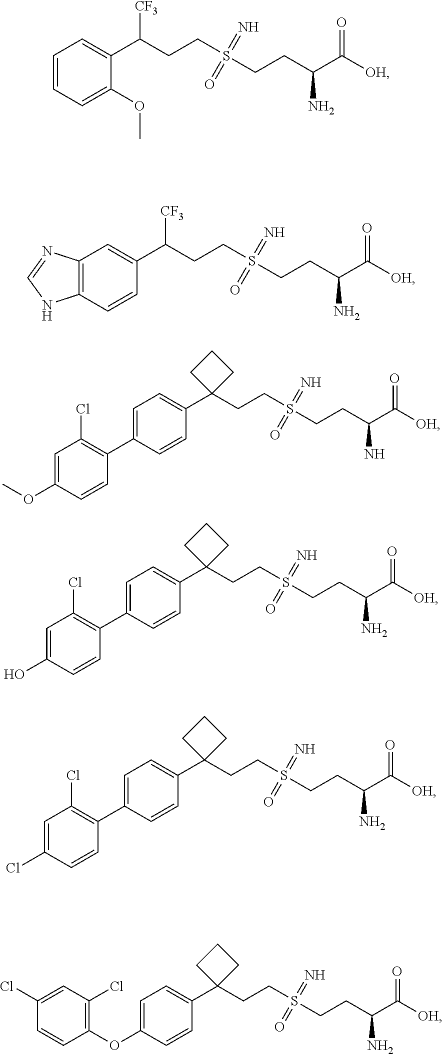 [SMILES: COc1ccccc1C(CCS(=N)(=O)CC[C@H](N)C(=O)O)C(F)(F)F ; N=S(=O)(CCC(c1ccc2[nH]cnc2c1)C(F)(F)F)CC[C@H](N)C(=O)O ; COc1ccc(-c2ccc(C3(CCS(=N)(=O)CC[C@H](N)C(=O)O)CCC3)cc2)c(Cl)c1 ; N=S(=O)(CC[C@H](N)C(=O)O)CCC1(c2ccc(-c3ccc(O)cc3Cl)cc2)CCC1 ; N=S(=O)(CC[C@H](N)C(=O)O)CCC1(c2ccc(-c3ccc(Cl)cc3Cl)cc2)CCC1 ; N=S(=O)(CC[C@H](N)C(=O)O)CCC1(c2ccc(Oc3ccc(Cl)cc3Cl)cc2)CCC1]

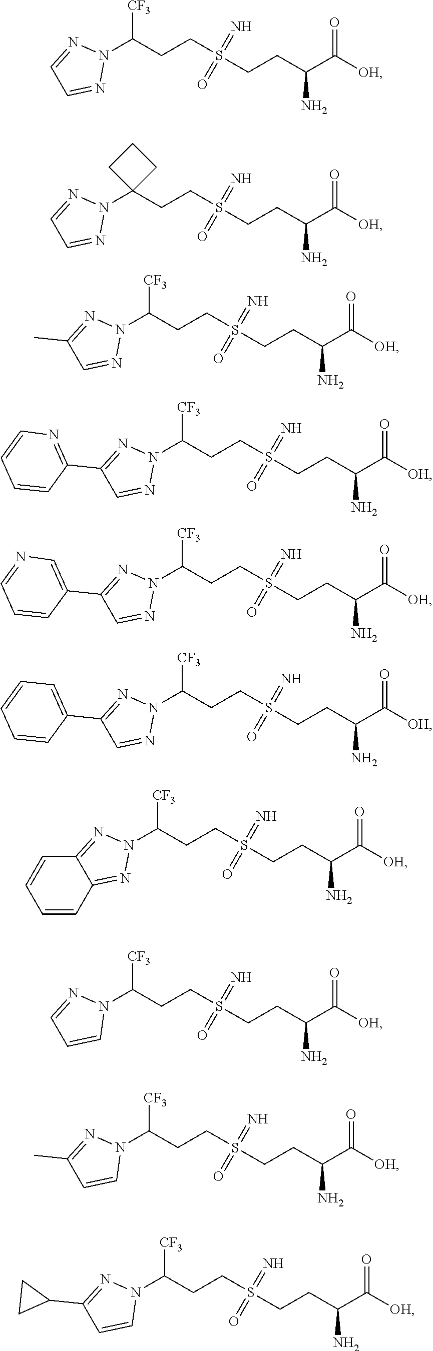

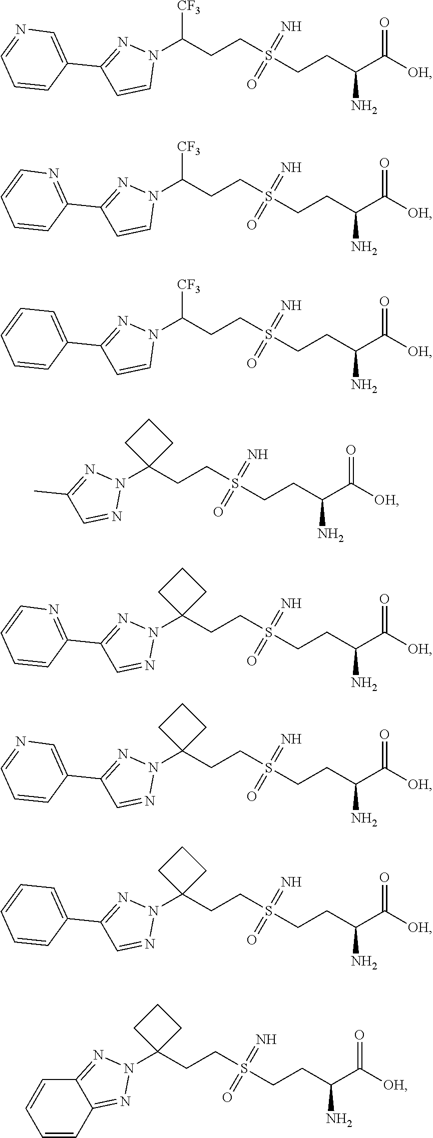

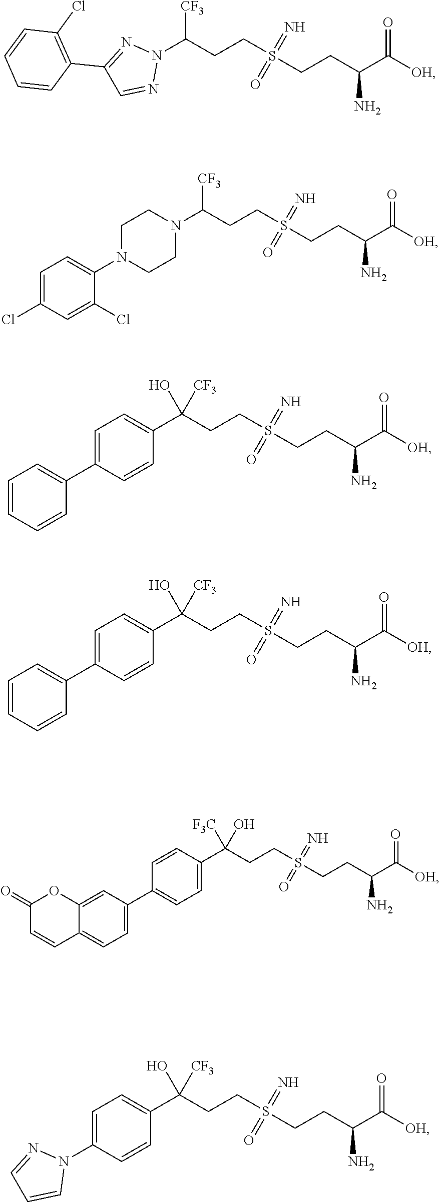 [SMILES: N=S(=O)(CCC(n1ncc(-c2ccccc2Cl)n1)C(F)(F)F)CC[C@H](N)C(=O)O ; N=S(=O)(CCC(N1CCN(c2ccc(Cl)cc2Cl)CC1)C(F)(F)F)CC[C@H](N)C(=O)O ; N=S(=O)(CC[C@H](N)C(=O)O)CCC(O)(c1ccc(-c2ccccc2)cc1)C(F)(F)F ; N=S(=O)(CC[C@H](N)C(=O)O)CCC(O)(c1ccc(-c2ccccc2)cc1)C(F)(F)F ; N=S(=O)(CC[C@H](N)C(=O)O)CCC(O)(c1ccc(-c2ccc3ccc(=O)oc3c2)cc1)C(F)(F)F ; N=S(=O)(CC[C@H](N)C(=O)O)CCC(O)(c1ccc(-n2cccn2)cc1)C(F)(F)F]

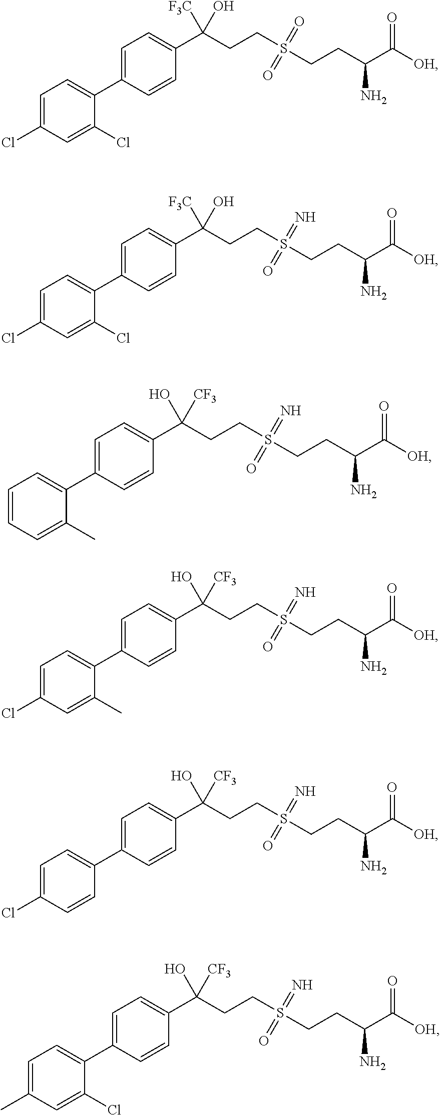 [SMILES: N[C@@H](CCS(=O)(=O)CCC(O)(c1ccc(-c2ccc(Cl)cc2Cl)cc1)C(F)(F)F)C(=O)O ; N=S(=O)(CC[C@H](N)C(=O)O)CCC(O)(c1ccc(-c2ccc(Cl)cc2Cl)cc1)C(F)(F)F ; Cc1ccccc1-c1ccc(C(O)(CCS(=N)(=O)CC[C@H](N)C(=O)O)C(F)(F)F)cc1 ; Cc1cc(Cl)ccc1-c1ccc(C(O)(CCS(=N)(=O)CC[C@H](N)C(=O)O)C(F)(F)F)cc1 ; N=S(=O)(CC[C@H](N)C(=O)O)CCC(O)(c1ccc(-c2ccc(Cl)cc2)cc1)C(F)(F)F ; Cc1ccc(-c2ccc(C(O)(CCS(=N)(=O)CC[C@H](N)C(=O)O)C(F)(F)F)cc2)c(Cl)c1]

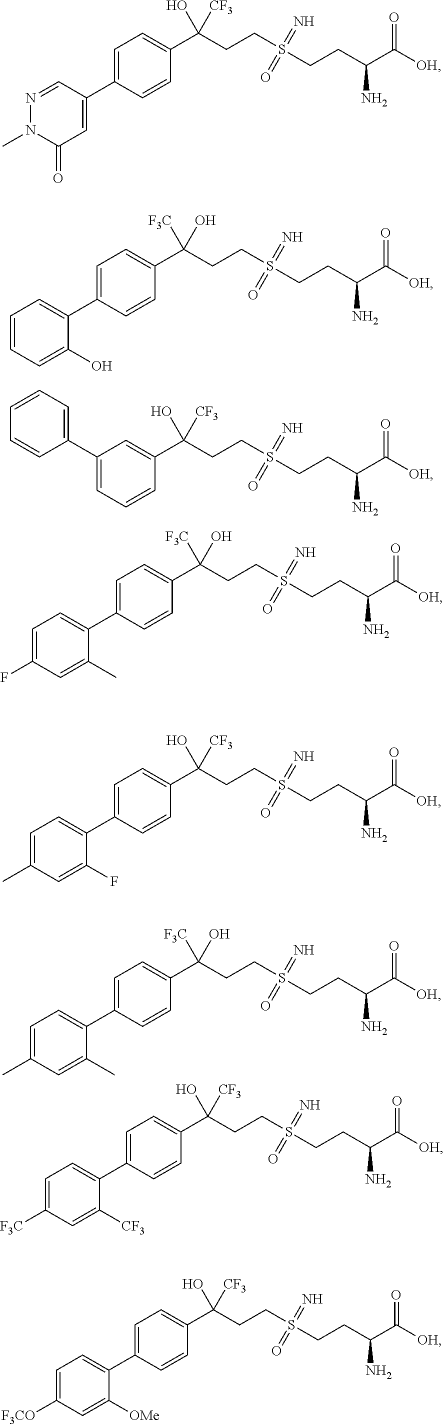

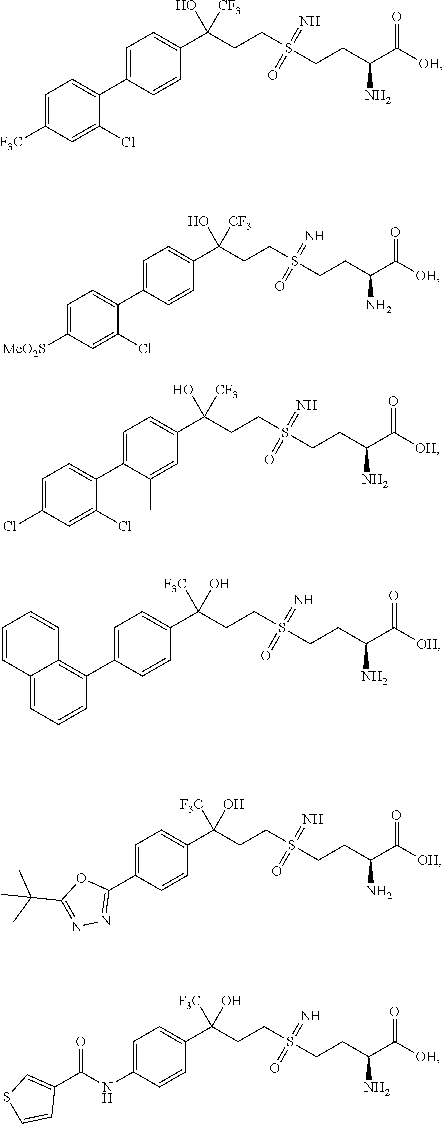 [SMILES: N=S(=O)(CC[C@H](N)C(=O)O)CCC(O)(c1ccc(-c2ccc(C(F)(F)F)cc2Cl)cc1)C(F)(F)F ; CS(=O)(=O)c1ccc(-c2ccc(C(O)(CCS(=N)(=O)CC[C@H](N)C(=O)O)C(F)(F)F)cc2)c(Cl)c1 ; Cc1cc(C(O)(CCS(=N)(=O)CC[C@H](N)C(=O)O)C(F)(F)F)ccc1-c1ccc(Cl)cc1Cl ; N=S(=O)(CC[C@H](N)C(=O)O)CCC(O)(c1ccc(-c2cccc3ccccc23)cc1)C(F)(F)F ; CC(C)(C)c1nnc(-c2ccc(C(O)(CCS(=N)(=O)CC[C@H](N)C(=O)O)C(F)(F)F)cc2)o1 ; N=S(=O)(CC[C@H](N)C(=O)O)CCC(O)(c1ccc(NC(=O)c2ccsc2)cc1)C(F)(F)F]

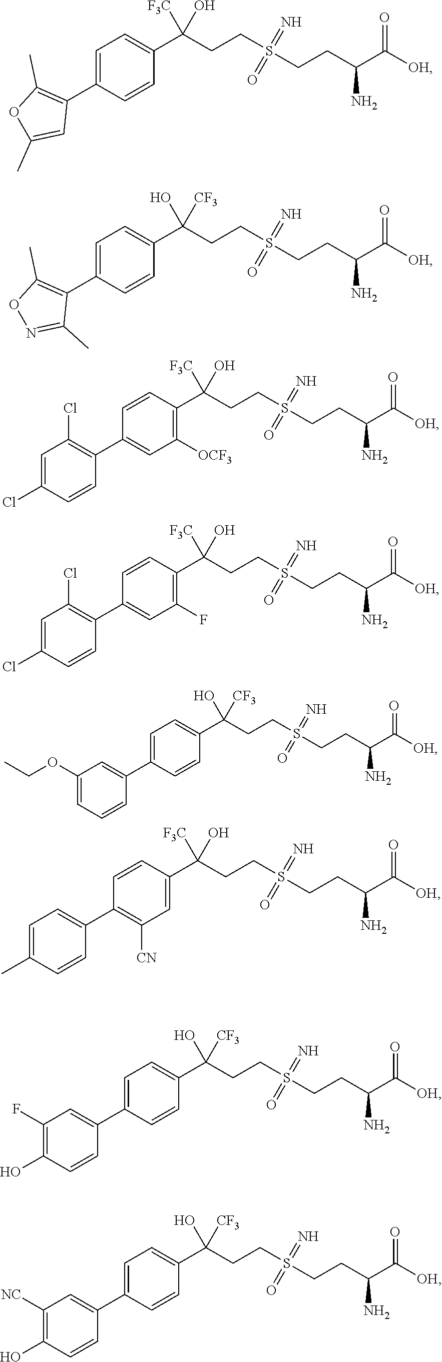

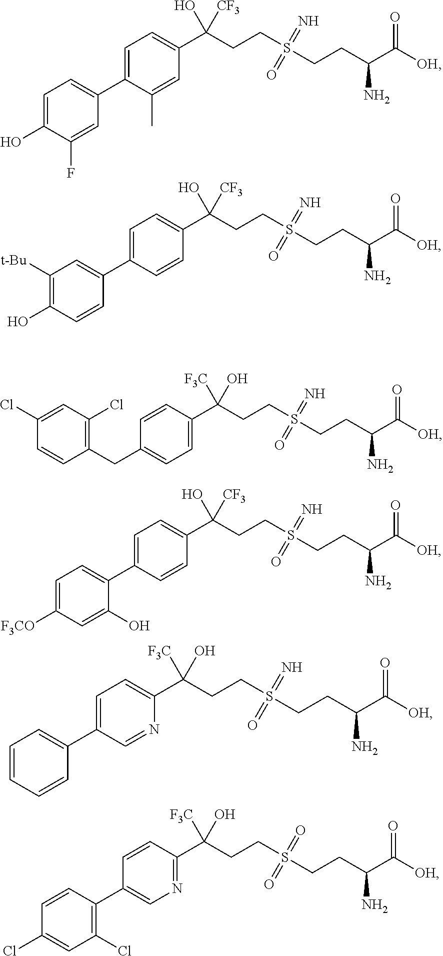

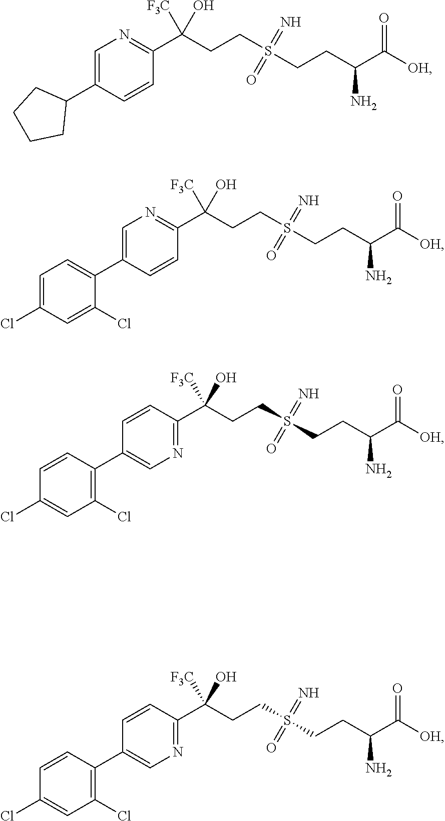 [SMILES: N=S(=O)(CC[C@H](N)C(=O)O)CCC(O)(c1ccc(C2CCCC2)cn1)C(F)(F)F ; N=S(=O)(CC[C@H](N)C(=O)O)CCC(O)(c1ccc(-c2ccc(Cl)cc2Cl)cn1)C(F)(F)F ; N=[S@@](=O)(CC[C@H](N)C(=O)O)CC[C@](O)(c1ccc(-c2ccc(Cl)cc2Cl)cn1)C(F)(F)F ; N=[S@](=O)(CC[C@H](N)C(=O)O)CC[C@](O)(c1ccc(-c2ccc(Cl)cc2Cl)cn1)C(F)(F)F]

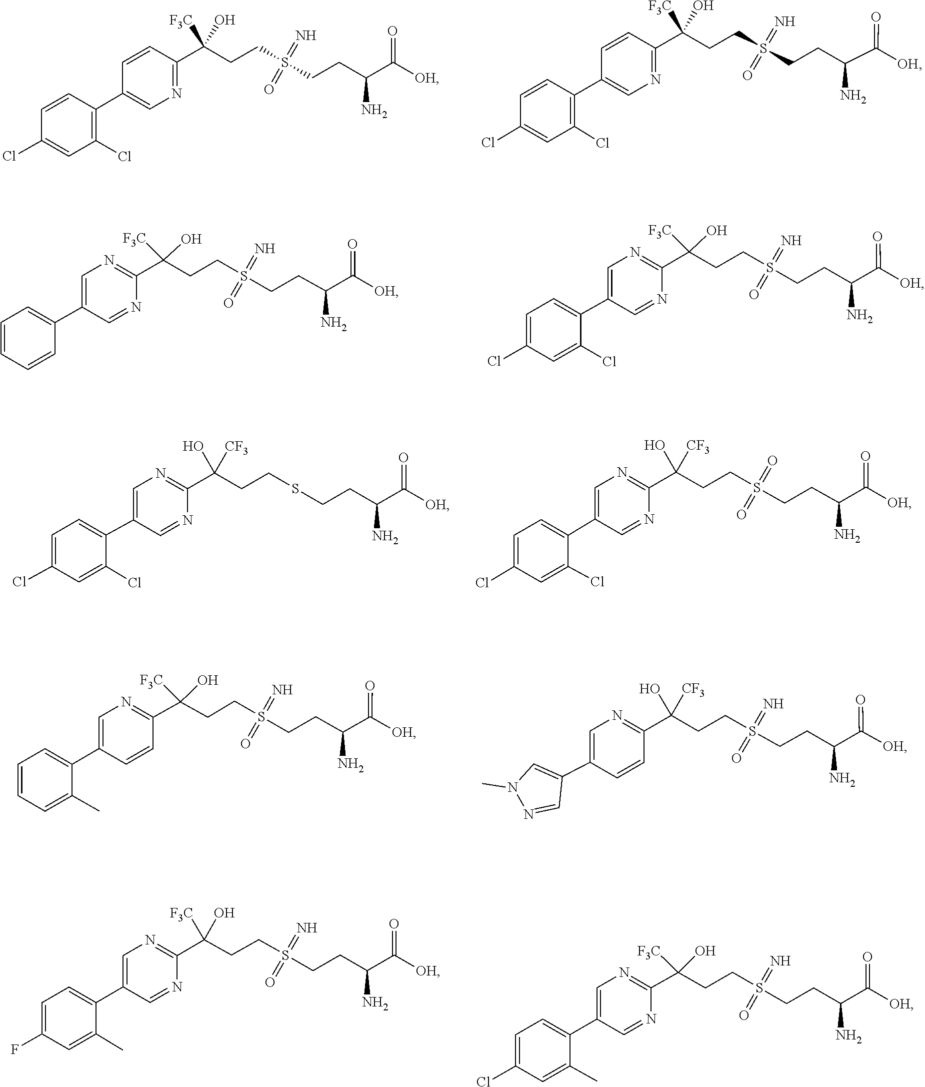

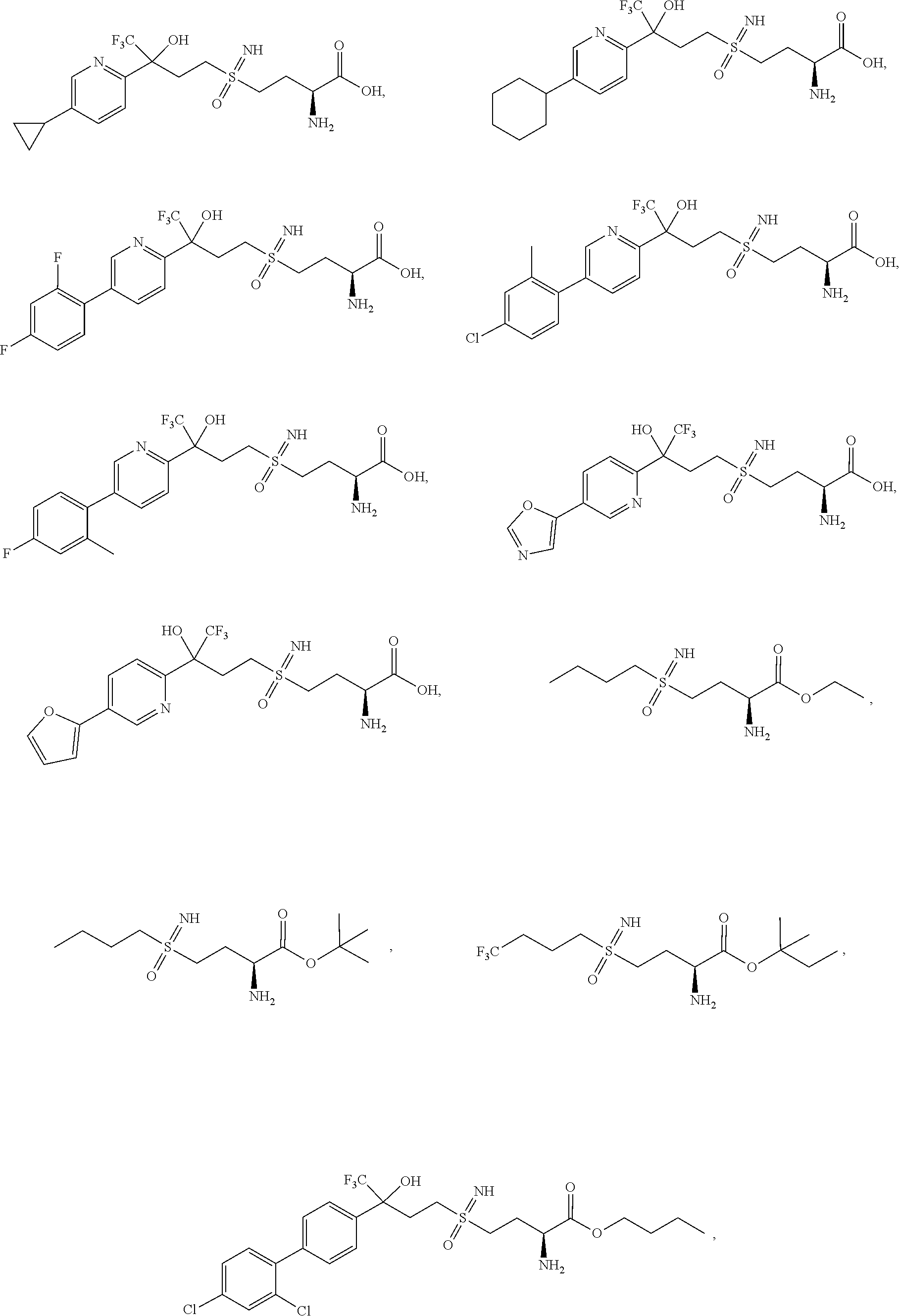

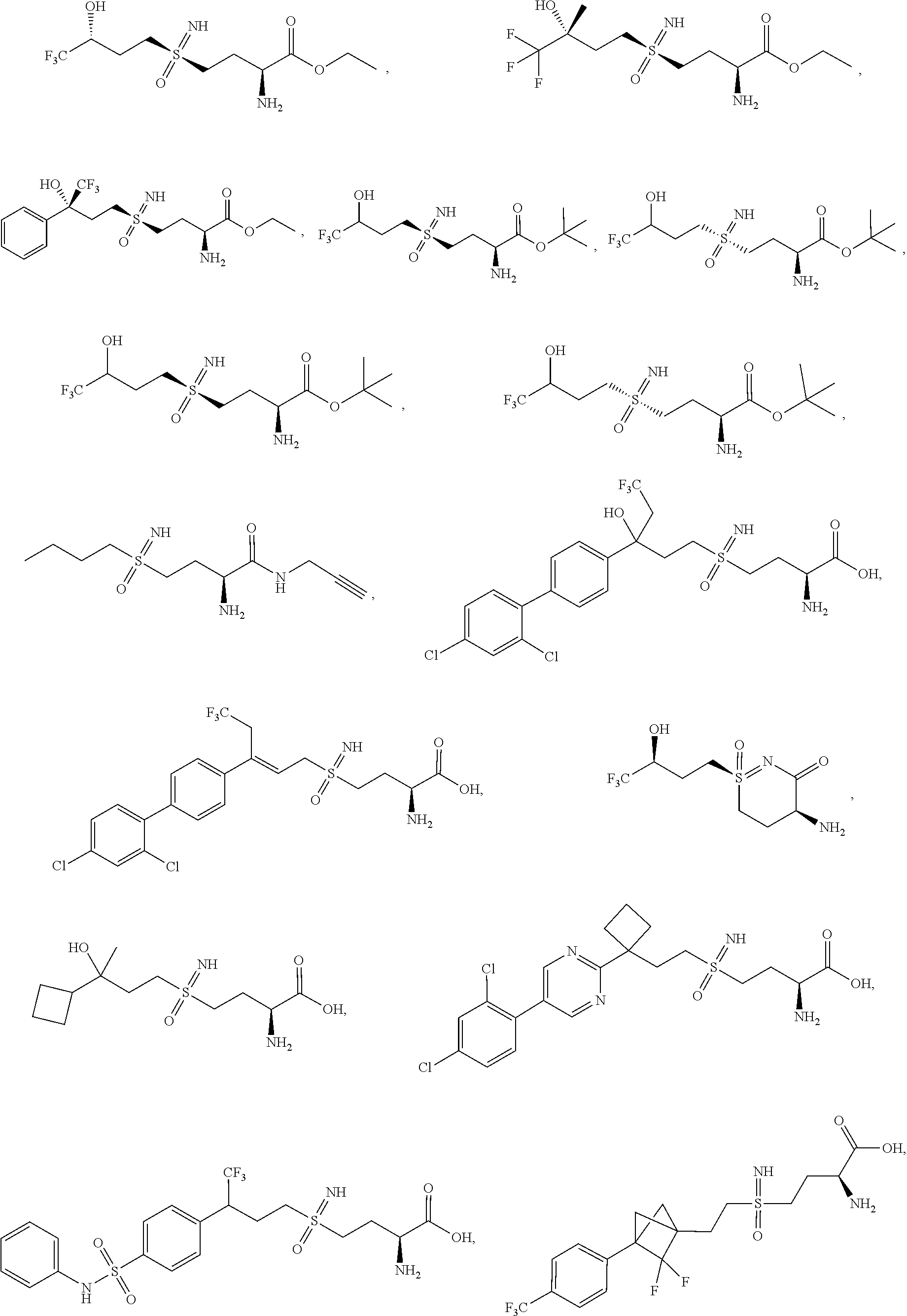

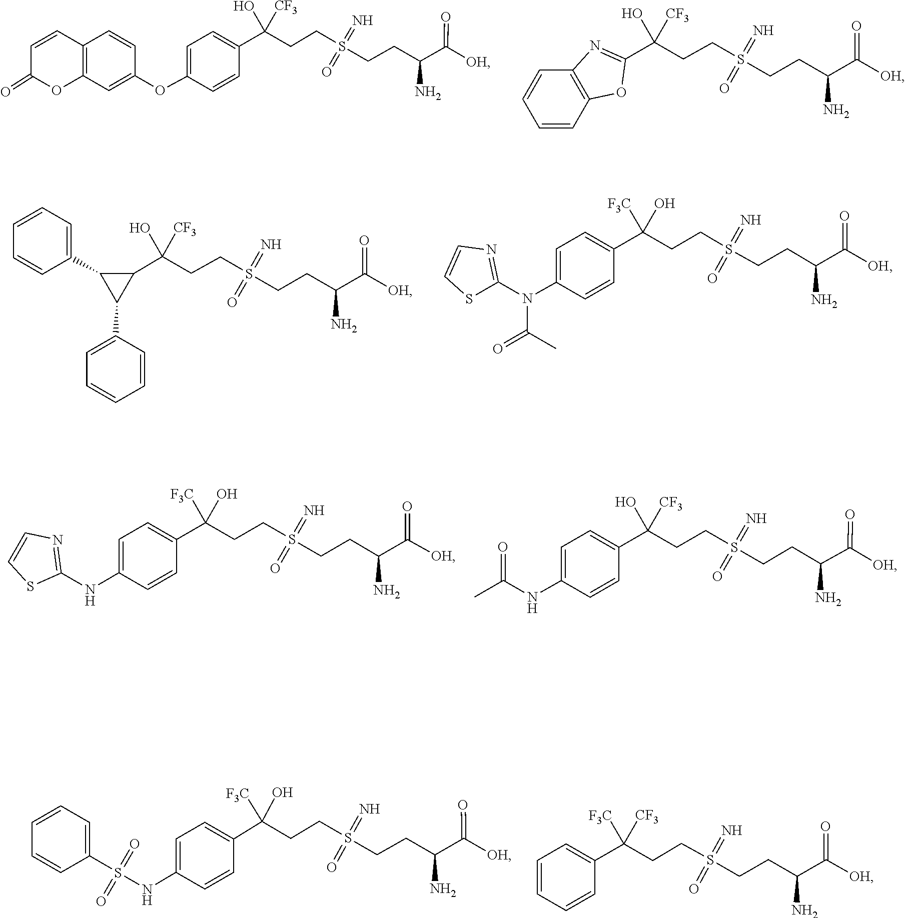

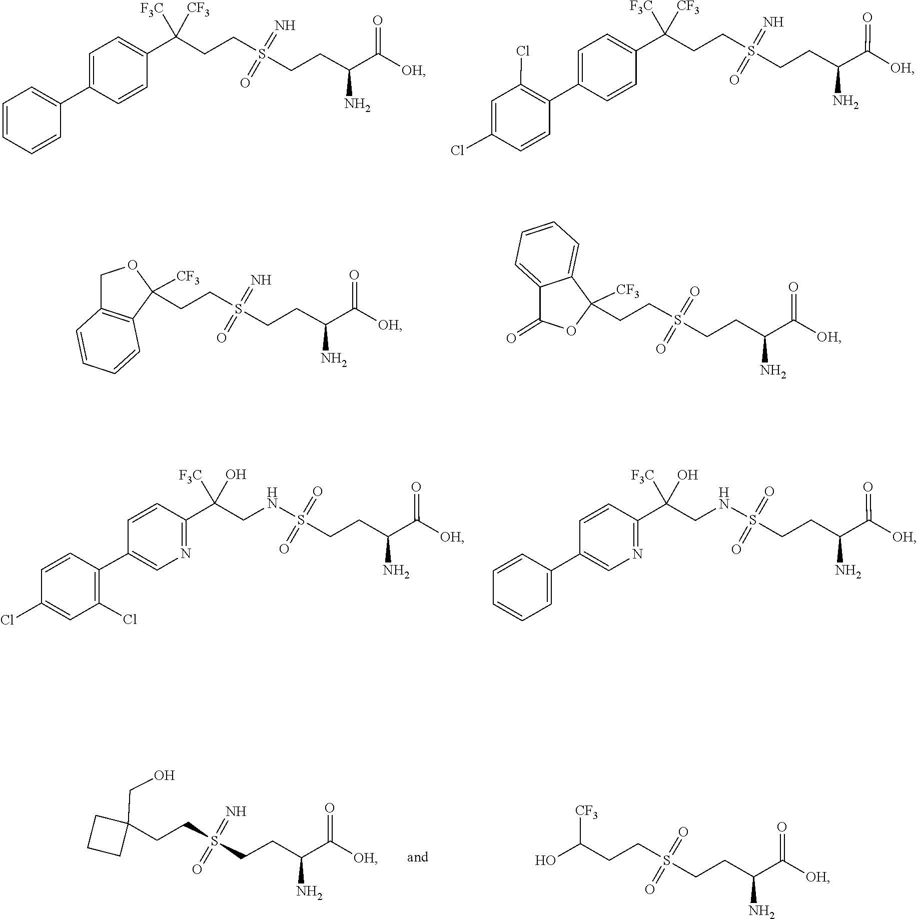

In some embodiments is a compound, a diastereomer or an enantiomer of the compound, or a pharmaceutically acceptable salt of any of the foregoing, or a deuterated derivative of any of the foregoing; wherein the compound is selected from the group consisting of:

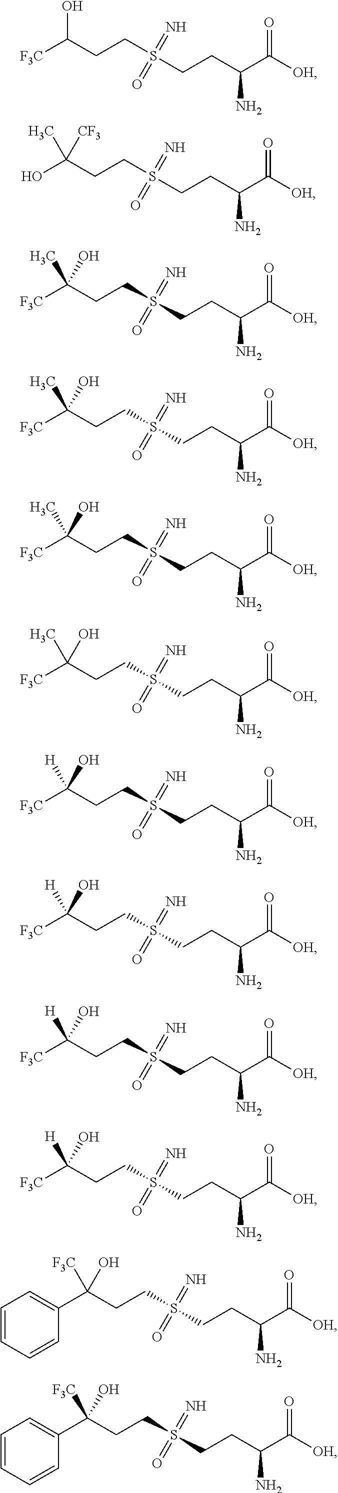

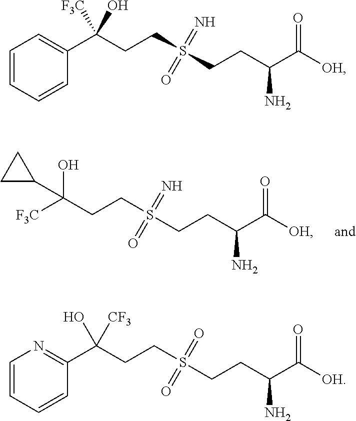 [SMILES: N=[S@@](=O)(CC[C@H](N)C(=O)O)CC[C@](O)(c1ccccc1)C(F)(F)F ; N=S(=O)(CC[C@H](N)C(=O)O)CCC(O)(C1CC1)C(F)(F)F ; N[C@@H](CCS(=O)(=O)CCC(O)(c1ccccn1)C(F)(F)F)C(=O)O]

In some embodiments described herein is a compound of Formula XVIII:

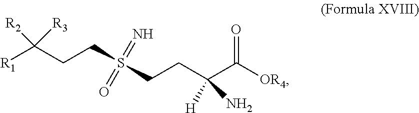

a diastereomer or an enantiomer of the compound of Formula XVIII, or a pharmaceutically acceptable salt of any of the foregoing, or a deuterated derivative of any of the foregoing; wherein in the compound of Formula XVIII:
   each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —CH₃, phenyl, —C(O)OR₅, —C(O)NH₂, —O—, —S—, —OH, —NH₂, —NH—, a halogen, —CF₃, —CN, —F, —Cl, —Br, —I;
   or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F, or R₁ and R₂ and R₃ are taken together to form a C₃-C₆ cycloheteroaryl; and wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

In some embodiments is a compound of Formula XIX:

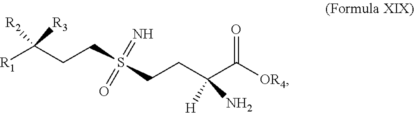

a diastereomer or an enantiomer of the compound of Formula XIX, or a pharmaceutically acceptable salt of any of the foregoing, or a deuterated derivative of any of the foregoing; wherein in the compound of Formula XIX:
   each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —C(O)OR₅, —C(O)NH₂, —O—, —S—, —OH, —NH₂, —NH—, a halogen, —CF₃, —CN, —F, —Cl, —Br, —I,
or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F,
or R₁ and R₂ and R₃ are taken together to form a C₃-C₆ cycloheteroaryl; and wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

In some embodiments is a compound of Formula XX:

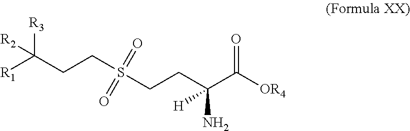

a diastereomer or an enantiomer of the compound of Formula XX, or a pharmaceutically acceptable salt of any of the foregoing, or a deuterated derivative of any of the foregoing; wherein in the compound of Formula XX:
   each R₁, R₂, or R₃ is independently: H, a C₁-C₄ linear or branched alkyl, a C₃-C₆ cycloalkyl, a C₆-C₁₀ aryl, a C₅-C₆ heteroaryl, a biphenyl, a halogenated biphenyl, a triazole, an isothiazole, an oxazoline, a C₁-C₆ linear or branched alkyl ether, —C(O)OR₅, —OH, —NH₂, —NH—, a halogen, —CF₃, —CN, —F,
   or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F; and
   wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, an oxazoline, a C₃-C₆ heteroaryl, and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

In some embodiments are pharmaceutical compositions comprising the compound of Formula XVIII, XIX, XX, or a compound described herein, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. In some embodiments, the pharmaceutical composition is in unit dose form. In some embodiments, the pharmaceutical composition further comprises an additional active agent or a pharmaceutically acceptable salt thereof. In some embodiments, the pharmaceutical composition is in the form of a powder, a tablet, a capsule, a liquid, or a gel. In some embodiments, the pharmaceutical composition is present in the pharmaceutical composition in an amount ranging from about 0.001 mg to about 25,000 mg. In some embodiments, are kits comprising a pharmaceutical composition described herein, and a container. In some embodiments, the container is a syringe. In some embodiments, the container is an IV bag. In some embodiments, the container is disposable. In some embodiments, the container is a single use container. In some embodiments, the container is a resealable container.

Also described herein are methods of treating a cancer in a subject. In some embodiments, the method comprises administering a pharmaceutical composition described herein to the subject in a therapeutically effective amount, thereby treating the cancer. In some embodiments, method comprising administering to the subject the compound of Formula XVIII, Formula XIX, Formula XX, or a compound described herein; the diastereomer or the enantiomer of any of the foregoing, or the pharmaceutically acceptable salt of any of the foregoing, or the deuterated derivative of any of the foregoing; in a therapeutically effective amount, thereby treating the cancer. In some embodiments, the cancer is a carcinoma, a sarcoma, or a melanoma. In some embodiments, the carcinoma is a liver carcinoma. In some embodiments, the cancer is a clear cell renal carcinoma or non-clear cell renal carcinoma. In some embodiments, the cancer is an SWI/SNF deficient-complex cancer. In some embodiments, the administering is selected from the group consisting of: oral, an injection; subcutaneous, intra-tumoral; systemic, local, intravenous, intraperitoneal, intramuscular, and any combination thereof.

Also described herein are methods of modulating ferroptosis in a tissue, the method comprising contacting the tissue with a pharmaceutical composition described herein in an amount effective to modulate the ferroptosis in the tissue. In some embodiments, the tissue is comprised in a subject. In some embodiments, the subject is a subject in need thereof. In some embodiments, the subject is a mammal. In some embodiments, the subject is a human. In some embodiments, the administering or the contacting is: as needed, once per day, twice per day, three times per day, once per week, once per two weeks, once per three weeks, once per month, once every six months, once per year, or for life. In some embodiments, the therapeutically effective amount, or the amount effective, ranges from about 0.001 mg to about 25,000 mg.

Also described herein are methods of treating a disease or condition in a subject, the method comprising administering a pharmaceutical composition described herein to the subject in a therapeutically effective amount, thereby treating the disease or condition. Also described herein are methods of treating a disease or condition in a subject, the method comprising administering a pharmaceutical composition described herein, the diastereomer or the enantiomer of any of the foregoing, or the pharmaceutically acceptable salt of any of the foregoing, or the deuterated derivative of any of the foregoing; in a therapeutically effective amount, thereby treating the disease or condition. In some embodiments, the disease or condition is a fibrosis or a kidney disorder.

Also described herein is a crystalline form of a compound described herein.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Compound Synthesis, Purification, and Characterization

Compounds herein and intermediates used in the preparation of compounds herein can be prepared using procedures shown in the following examples and related procedures. The methods and conditions used in these examples, and the actual compounds prepared in these examples, are not meant to be limiting, but are meant to demonstrate how the compounds of the current disclosure can be prepared. Starting materials and reagents used in these examples, when not prepared by a procedure described herein, are generally either commercially available, or are reported in the chemical literature, or may be prepared by using procedures described in the chemical literature. Column chromatography was performed with pre-packed silica gel cartridges or manually loaded column chromatography systems. Preparative high performance liquid chromatography (HPLC) was performed using a reverse phase column as indicated of a size appropriate to the quantity of material being separated, generally eluting with a gradient of increasing concentration of methanol or acetonitrile in water, also containing 0.05% or 0.1% trifluoroacetic acid or 10 mM ammonium acetate, at a rate of elution suitable to the column size and separation to be achieved. In some instances, chiral chromatography was performed to separate stereoisomers. Chemical names were determined using ChemDraw Ultra, version 20.1 (CambridgeSoft). The following abbreviations are used:
Å angstroms
AIBN 2,2′-azobis(2-methylpropionitrile)
aq. aqueous
Ac₂O acetic anhydride
brine saturated aqueous sodium chloride
Boc tert-butoxy carbonyl
BOP benzotriazol-1-yloxytris-(dimethylamino)-phosphonium hexafluorophosphate
bpy 2,2′-bipyridine
Bn Benzyl
Bz Benzoyl
Cbz Benzyloxycarbonyl
DBU 1,8-diazabicyclo[5.4.0]undec-7-ene
DCM dichloromethane
DIPEA diisopropylethylamine
DMAP 4-dimethylaminopyridine
DMF N,N-dimethylformamide
DMFDMA N,N-dimethylformamide dimethyl acetal
DMSO dimethyl sulfoxide
EtOAc ethyl acetate
EtOH ethanol
Et₃SiH triethylsilane
FA formic acid
FMOC 9-Fluorenylmethyloxycarbonyl
g gram(s)
h hour(s)
HATU 1-[bis(dimethylamino)methylene]-1H-1,2,3-triazolo[4,5-b]pyridinium 3-oxid hexafluorophosphate
HPLC High Performance Liquid Chromatography
i-PrOH iso-propanol
KHMDS potassium bis(trimethylsilyl)amide
LAH lithium aluminum hydride
LCMS Liquid Chromatography-Mass Spectroscopy
m-CPBA meta-chloro perbenzoic acid
MeCN acetonitrile
MeOH methanol
MHz megahertz
MS molecular sieves
MsCl methanesulfonyl chloride
MTBE tert-butyl methyl ether
NaOAc sodium acetate
NH₄OAc ammonium acetate
NIS N-iodosuccinimide
NMO N-methylmorpholine N-oxide
NMR Nuclear Magnetic Resonance
Ns Nosyl
Pd₂(dba)₃ tris(dibenzylideneacetone)dipalladium(0)
Pd(dppf)Cl₂ [1,1′-bis(diphenylphosphino)ferrocene]dichloropalladium(II)
Pd(OAc)₂ palladium acetate
Pd(PPh₃)₄ tetrakis(triphenylphosphine)palladium(0)
Pd(PPh₃)₂Cl₂ bis(triphenylphosphine)palladium(II) dichloride
pet ether petroleum ether
PIDA (diacetoxyiodo)benzene
PhI(OAc)₂ (diacetoxyiodo)benzene
PPTS pyridinium para-toluene sulfonic acid
p-TsOH para-toluenesulfonic acid
Ts tosyl
TsCl para-toluenesulfonyl chloride
[Rh(COD)CI]₂ chloro(1,5-cyclooctadiene)rhodium(I) dimer
SEM 2-(trimethylsilyl)ethoxymethyl
t-BuOH tert-butanol
TBAF tetra-N-butylammonium fluoride
TEA triethylamine
TFA trifluoroacetic acid
TFAA trifluoroacetic anhydride
THE tetrahydrofuran
TIPS Triisopropylsilyl ether
TLC thin layer chromatography
TMS trimethylsilyl
TMSCF₃ trimethyl(trifluoromethyl)silane
TPAP tetrapropylammonium perruthenate

### HPLC Conditions:

A. Phenomnex Luna C18, 2×50 mm, 5 microns, column temp 40° C., 0-30% 13 for 3 min then 100% B for 1 min. Solvent A 0.04% TFA in water, Solvent B, 0.02% TFA in MeCN, 1 mL/min.
B. XBridge C18, 2.1×50 mm, 5 microns, column temp 40° C., 0-95% B for 3.85 min. Solvent A 10 mM NH₄CO₃ in water, Solvent B, MeCN, I mL/min or preferably, 0.8 mL/min.
C. Phenomnex Luna C18, 2×50 mm, 5 microns, column temp 40° C., 5-95% B for 3 min then 95% B for 1 min. Solvent A 0.04% TFA in water, Solvent B, 0.02% TFA in MeCN, 1 mL/min.
D. Phenomnex Luna C18, 2×50 mm, 5 microns, column temp 40° C., 0-60% 13 for 5 min then to 100% B for 1.5 min. Solvent A 0.04% TFA in water, Solvent B, 0.02% TFA in MeCN, 0.8 mL/min.
E. Phenomnex Luna C18, 2×50 mm, 5 microns, column temp 40° C., 0-30% B for 3 min then 30% B for 1 min. Solvent A 0.04% TFA in water, Solvent B, 0.02% TFA in MeCN, 1 mL/min.
F. Kinetix C18, 2.1×50 mm, 5 microns, column temp 40° C., 5-95% B for 3 min then 95% B for 1 min. Solvent A 0.04% TFA in water, Solvent 13, 0.02% TFA in MeCN. 1 mL/min.
G. XBridge C18, 2.1×50 mm, 5 microns, column temp 40° C., 0-30% B for 3.4 min then 100% B for 0.45 min. Solvent A 10 mM NH₄CO₃ in water, Solvent B, MeCN, 0.6 mL/min.
H. Kinetix C18, 2×50 mm, 5 microns, column temp 40° C., 0-30% B for 3 min then 100% B for 1 min. Solvent A 0.04% TFA in water, Solvent B, 0.02% TFA in MeCN. 1 mL/min.
I. XBridge C18, 2.1×50 mm, 5 microns, column temp 40° C., 0-60% B for 4 min then 60% B for 2 min. Solvent A 10 mM NH₄CO₃ in water, Solvent B, MeCN, 0.6 mL/min.
J. Kinetix EVO C18, 2×30 mm, 5 microns, column temp 40° C., 5-95% B for 0.7 min then 95% B for 0.46 min. SolventA 0.04% TFA in water, Solvent B, 0.02% TFA in MeCN. 1.5 mL/min.
K. XBridge C18, 2.1×50 mm, 5 microns, column temp 40° C., 0-60% B for 4 min then 60% B for 2 min. Solvent A 10 mM NH₄CO₃ in water, Solvent B, MeCN, 0.6 mL/min.
L. Chomolith Flash RB-18e C18, 2×25 mm, column temperature: 40° C., 0-30% B for 3.5 min then 30% B for 0.3 min. Solvent A 0.04% TFA in water, Solvent B, 0.02% TFA in MeCN at a flow rate of 0.8 mL/min.
M. Merck Chomolith Flash RP-18e, column temperature: 40° C., 0-30% B for 1.2 min then 30% B for 0.4 min. Solvent A 0.04% TFA in water, Solvent B 0.02% TFA in MeCN at a flow rate of 1.5 mL/min.
N. Agilent poroshell 120 EC-CIS 3.0×50 mm, 2.7 microns, column temperature: 45° C., 5-99% B for 3 min then 99% B for 1 min. Solvent A 0.04% TFA in water, Solvent B, 0.02% TFA in MeCN at a flow rate of 1 mL/min.
O. Waters XSelect HSS T3 4.6×50 mm, 3.5 microns, column temperature: 40° C., 0-30% B for 3 min then 100% B for 1 min. Solvent A 0.04% TFA in water, Solvent B, 0.02% TFA in MeCN at a flow rate of 1 mL/min.
P. Agilent ZORBAX SB-Aq, 2.1×50 mm, 5 microns, column temperature: 45° C., 0-80% B for 3.4 min then 100% B for 0.5 min. Solvent A 0.04% TFA in water, Solvent B, 0.02% TFA in MeCN at a flow rate of 0.6 mL/min.
Q. Kinetex® EVO C18 2.1×30 mm 5 microns, column temperature: 45° C., 0-60% B for 3.6 min then 60% B for 0.25 min. Solvent A 0.04% TFA in water, Solvent B, 0.02% TFA in MeCN at a flow rate of 0.6 mL/min.
R. Waters XSelect HSS T3 4.6×50 mm, 3.5 microns, column temperature: 40° C., 0-60% B for 3 min then 100% B for 1 min. Solvent A 0.04% TFA in water, Solvent B, 0.02% TFA in MeCN at a flow rate of 1 mL/min.
S. Agilent ZORBAX RX-SIL, 4.6×150 mm, 5 microns, column temperature: 40° C., 0-85% B for 7 min. Solvent A 0.04% TFA in water, Solvent B, 0.02% TFA in MeCN I mL/min.
T. Agilent XBridge C18, 2.1×50 mm, 5 microns, column temperature: 40° C., 0-60% B for 4 min, then 60% B for 2 min. Solvent A, 10 mM NH₄CO₃ in water, Solvent B, MeCN at a flow rate of 0.8 mL/min.
U. Halo C18, 3.0×30 mm, 5 microns, column temperature: 40° C., 10-100% B for 0.5 min then 100% B for 0.4 min. Solvent A, 0.04% TFA in water, Solvent B, 0.02% TFA in MeCN at a flow rate of 2 mL/min.

### Exemplary Embodiment 1a

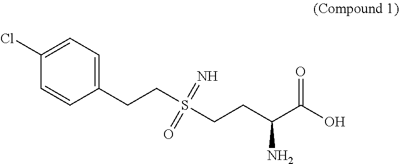 [SMILES: N=S(=O)(CCc1ccc(Cl)cc1)CC[C@H](N)C(=O)O]

### (2S)-2-amino-4-(2-(4-chlorophenyl)ethylsulfonimidoyl)butanoic acid

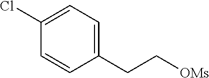 [SMILES: CS(=O)(=O)OCCc1ccc(Cl)cc1]

To a mixture of (chlorophenyl)ethan-1-ol (100 mg, 6.39 mmol) and TEA (19.2 mmol, 2.67 mL) in DCM (10 mL) was added MsCl (19.2 mmol, 1.48 mL) at 0° C. The mixture was stirred at 0-25° C. for 16 h. The mixture was quenched with water and extracted with DCM. The organic phase was dried over Na₂SO₄, filtered, and concentrated. The residue was purified by silica gel chromatography (petroleum ether/ethyl acetate=100/1, 1/1) to afford 4-chlorophenethyl methanesulfonate (1 g, 66.7% yield) as colorless oil. ¹H NMR (400 MHz, CDCl₃) δ 7.34-7.28 (m, 2H), 7.18 (d, J=8.44 Hz, 2H), 4.40 (t, J=6.79 Hz, 2H), 3.04 (t, J=6.79 Hz, 2H), 2.90 (s, 3H).

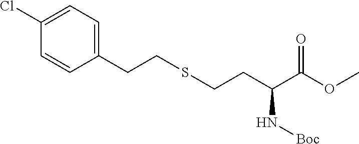 [SMILES: COC(=O)[C@H](CCSCCc1ccc(Cl)cc1)NC(=O)OC(C)(C)C]

To a mixture of 4-chlorophenethyl methanesulfonate (375 mg, 1.6 mmol) and tert-butyl (tert-butoxycarbonyl)-L-homocysteinate (400 mg, 1.6 mmol) in DMF was added KI (2.4 mmol) and K₂CO₃ (4.8 mmol) under Ar. The mixture was stirred at 25-70° C. for 16 h, quenched with water, and extracted with ethyl acetate. The combined organic phase was washed with brine, dried over Na₂SO₄, filtered, and concentrated. The residue was purified by prep-TLC (Petroleum ether: ethyl acetate=2:1, R_{f}=0.50) to afford (S)-methyl 2-((tert-butoxycarbonyl)amino)-4-((4-chlorophenethyl)thio)butanoate (0.37 g, 60% yield) as colorless oil. ¹H NMR (400 MHz, CDCl₃-d) δ 7.29-7.25 (m, 3H), 7.14 (d, J=8.44 Hz, 2H), 5.10 (br d, J=6.97 Hz, 1H), 4.42 (br d, J=4.65 Hz, 1H), 3.75 (s, 3H), 2.90-2.81 (m, 2H), 2.80-2.71 (m, 2H), 2.61-2.51 (m, 2H), 2.18-2.06 (m, 1H), 1.98-1.85 (m, 1H), 1.45 (s, 9H).

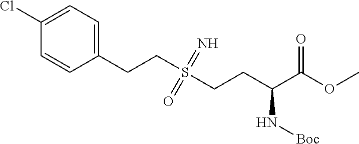 [SMILES: COC(=O)[C@H](CCS(=N)(=O)CCc1ccc(Cl)cc1)NC(=O)OC(C)(C)C]

A mixture of (S)-methyl-2-((tert-butoxycarbonyl)amino)-4-((4-chlorophenethyl)thio)butanoate (170 mg, 0.44 mmol), PhI(OAc)₂ (1.1 mmol) and ammonium carbamate (2.2 mmol) in i-PrOH (3 mL) was stirred at 25° C. for 16 h. The mixture was quenched with water and extracted with ethyl acetate. The combined organic phase was washed with brine, dried over anhydrous Na₂SO₄, filtered, and concentrated. The residue was purified by silica gel chromatography (column height: 250 mm, diameter: 100 mm, 100-200 mesh silica gel, petroleum ether/ethyl acetate=5/1, 0/1) to afford (2S)-methyl 2-((tert-butoxycarbonyl)amino)-4-(2-(4-chlorophenyl)ethylsulfonimidoyl)butanoate (0.1 g, 56% yield) as colorless oil. ¹H NMR (400 MHz, CDCl₃-d) δ 7.31 (d, J=8.33 Hz, 1H), 7.35-7.28 (m, 1H), 7.19 (d, J=8.33 Hz, 2H), 5.31 (s, 1H), 4.49 (s, 1H), 4.42 (br s, 1H), 3.78 (s, 3H), 3.47-3.28 (m, 2H), 3.17 (br d, J=7.89 Hz, 4H), 2.51-2.34 (m, 1H), 2.31-2.13 (m, 1H), 1.45 (s, 9H).

A mixture of (2S)-methyl 2-((tert-butoxycarbonyl)amino)-4-(2-(4-chlorophenyl)ethylsulfonimidoyl)butanoate (40.0 mg, 95.5 mmol) in HCl (6 M, 1 mL) was stirred at 50° C. for 16 h. The mixture was dried by freeze drying to give (2S)-2-amino-4-(2-(4-chlorophenyl)ethylsulfonimidoyl)butanoic acid (28.8 mg, 80% yield, HCl) as yellow oil. LCMS: Rt=2.445 min, (ES⁺) m/z (M+H)⁺=305.0, HPLC Conditions: A; ¹H NMR (400 MHz, D₂O) δ 7.38-7.32 (m, 2H), 7.31-7.24 (m, 2H), 4.09 (br s, 1H), 4.07-3.98 (m, 2H), 3.91-3.66 (m, 2H), 3.21 (br t, J=7.15 Hz, 2H), 2.38 (br d, J=6.72 Hz, 2H).

### Exemplary Embodiment 1b

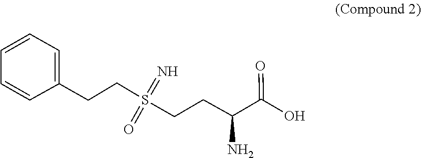 [SMILES: N=S(=O)(CCc1ccccc1)CC[C@H](N)C(=O)O]

### (2S)-2-amino-4-(2-phenylethylsulfonimidoyl)butanoic acid

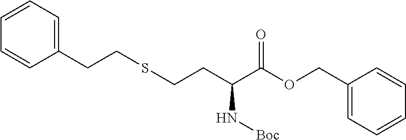 [SMILES: CC(C)(C)OC(=O)N[C@@H](CCSCCc1ccccc1)C(=O)OCc1ccccc1]

To a solution of benzyl (2S)-2-(tert-butoxycarbonylamino)-4-sulfanyl-butanoate (0.800 g, 2.46 mmol) and 2-bromoethylbenzene (500 mg, 365 mL, 2.7 mmol) in DMF (10 mL) was added K₂CO₃ (1.02 g, 7.38 mmol). The mixture was stirred at 20° C. for 1 h. Water (30 mL) was added, and the product was extracted with MTBE (30 mL). The organic layers were separated and dried over Na₂SO₄, filtered, and concentrated. The residue was purified by column chromatography (SiO₂, Petroleum ether: ethyl acetate=9:1 to 7:3) to give (S)-benzyl 2-((tert-butoxycarbonyl)amino)-4-(phenethylthio)butanoate (1.1 g, crude) as colorless oil. ¹H NMR (400 MHz, CDCl₃-d) δ 7.41-7.27 (m, 7H), 7.26-7.12 (m, 3H), 5.18 (q, J=12.5 Hz, 3H), 4.50-4.36 (m, 1H), 2.89-2.79 (m, 2H), 2.77-2.67 (m, 2H), 2.57-2.45 (m, 2H), 2.17-2.06 (m, 1H), 1.99-1.83 (m, 1H), 1.44 (s, 9H).

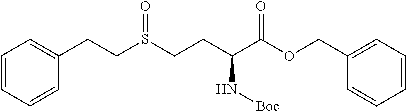 [SMILES: CC(C)(C)OC(=O)N[C@@H](CCS(=O)CCc1ccccc1)C(=O)OCc1ccccc1]

To a solution of (S)-benzyl 2-((tert-butoxycarbonyl)amino)-4-(phenethylthio)butanoate (1.00 g, 2.33 mmol) in DCM (10 mL) was added m-CPBA (473 mg, 2.33 mmol, 85% purity). The mixture was stirred at 20° C. for 1 h. The reaction was quenched by NaHCO₃ (20 mL) and then extracted with DCM (20 mL×2). The combined organic phase was washed with Na₂SO₃ (10 mL), dried over anhydrous Na₂SO₄, filtered, and concentrated in vacuo. The residue was purified by column chromatography (SiO₂, Petroleum ether: ethyl acetate=1:1 to 1:2) to give (2S)-benzyl 2-((tert-butoxycarbonyl)amino)-4-(phenethylsulfinyl)butanoate (910 mg, 88% yield) as colorless oil. ¹H NMR (400 MHz, CDCl₃-d) δ 7.39-7.29 (m, 7H), 7.26-7.19 (m, 3H), 5.35-5.10 (m, 3H), 4.45 (br d, J=4.3 Hz, 1H), 3.18-2.99 (m, 2H), 2.99-2.79 (m, 2H), 2.77-2.59 (m, 2H), 2.45-2.26 (m, 1H), 2.23-2.07 (m, 1H), 1.43 (s, 9H).

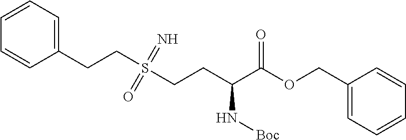 [SMILES: CC(C)(C)OC(=O)N[C@@H](CCS(=N)(=O)CCc1ccccc1)C(=O)OCc1ccccc1]

To a solution of (2S)-benzyl 2-((tert-butoxycarbonyl)amino)-4-(phenethylsulfinyl)butanoate (910 mg, 2.04 mmol) in MeOH (10 mL) was added ammonium carbamate (797 mg, 10.2 mmol) and PhI(OAc)₂ (1.97 g, 6.13 mmol). The mixture was stirred at 20° C. for 2 h. The mixture was concentrated to give the crude product. Water (20 mL) was added to the residue and the product was extracted with ethyl acetate (20 mL×2). The combined organic phase was dried over anhydrous Na₂SO₄, filtered, and concentrated in vacuo. The residue was purified by column chromatography (SiO₂, Petroleum ether: ethyl acetate=1:1 to 1:2) to give (2S)-benzyl 2-((tert-butoxycarbonyl)amino)-4-(2-phenylethylsulfonimidoyl)butanoate (620 mg, 66% yield) as colorless oil. ¹H NMR (400 MHz, CDCl₃-d) δ 7.42-7.27 (m, 8H), 7.26-7.19 (m, 2H), 5.37-5.12 (m, 3H), 4.50-4.33 (m, 1H), 3.38-2.90 (m, 6H), 2.48-2.32 (m, 1H), 2.23-2.11 (m, 1H), 1.43 (s, 9H).

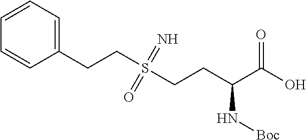 [SMILES: CC(C)(C)OC(=O)N[C@@H](CCS(=N)(=O)CCc1ccccc1)C(=O)O]

To a solution of (2S)-benzyl 2-((tert-butoxycarbonyl)amino)-4-(2-phenylethylsulfonimidoyl)butanoate (0.40 g, 868 mmol) in THE (5 mL) and H₂O (1 mL) was added LiOH·H₂O (72.9 mg, 1.74 mmol). The mixture was stirred at 60° C. for 2 h. The reaction mixture was concentrated to afford a residue. Water (10 mL) was added, and the aqueous layer was extracted with DCM (10 mL). The aqueous phase was adjusted to pH˜4 with 2N HCl and extracted with DCM (10 mL×3). The combined organic extracts were concentrated to afford (2S)-2-((tert-butoxycarbonyl)amino)-4-(2-phenylethylsulfonimidoyl)butanoic acid (274 mg, 85% yield) as a white solid. ¹H NMR (400 MHz, CDCl₃-d) δ7.39-7.27 (m, 3H), 7.25 (br s, 2H), 5.60 (br d, J=6.4 Hz, 1H), 4.57-4.27 (m, 1H), 3.58-3.39 (m, 2H), 3.33-3.02 (m, 4H), 2.45-2.14 (m, 2H), 1.45 (s, 9H).

To a solution of (2S)-2-((tert-butoxycarbonyl)amino)-4-(2-phenylethylsulfonimidoyl)butanoic acid (80.0 mg, 216 mmol) in HCl/dioxane (4 M, 3 mL) was stirred at 20° C. for 4 h. The mixture was concentrated, water was added and washed with DCM (2 mL×3). The aqueous phase was concentrated to give (2S)-2-amino-4-(2-phenylethylsulfonimidoyl)butanoic acid (76.31 mg, crude, HCl) as a white solid. LCMS: Rt=1.672 min., (ES⁺) m/z (M+H)⁺=271.1, HPLC Conditions: A; ¹H NMR (400 MHz, D₂O) δ 7.55-7.17 (m, 5H), 4.17-3.94 (m, 3H), 3.86-3.67 (m, 2H), 3.32-3.20 (m, 2H), 2.48-2.29 (m, 2H).

The compounds described in Table 5 were prepared using the methods described in above.

TABLE 5
Characterization of compounds 3-48

| Compound | | MS | HPLC | HPLC |
|---|---|---|---|---|
| # | Structure | (M + H)⁺ | rt | cond. |
| 3 | 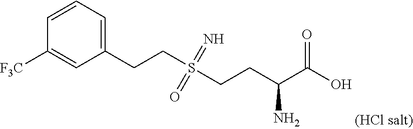 [SMILES: N=S(=O)(CCc1cccc(C(F)(F)F)c1)CC[C@H](N)C(=O)O] | 339.0 | 1.82 | C |
| | ¹H NMR (400 MHz, D₂O) δ = 7.73-7.53 (m, | | | |
| | 4H), 4.03-3.82 (m, 3H), 3.63 (s, 2H), 3.30 (t, | | | |
| | 2H), 2.40 (q, 2H) | | | |
| 4 | 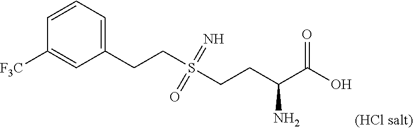 [SMILES: N=S(=O)(CCc1cccc(C(F)(F)F)c1)CC[C@H](N)C(=O)O] | 355.0 | 3.09 | E |
| | ¹H NMR (400 MHz, D₂O) δ 7.30-7.20 (m, 2H), | | | |
| | 7.12 (br s, 1H), 4.11 (dt, 3H), 3.95-3.81 (m, 2H), | | | |
| | 3.25 (br t, 2H), 2.52-2.37 (m, 2H) | | | |
| 5 | 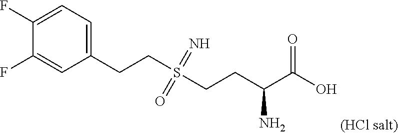 [SMILES: N=S(=O)(CCc1ccc(F)c(F)c1)CC[C@H](N)C(=O)O] | 307.0 | 2.29 | A |
| | ¹H NMR (400 MHz, D₂O) δ 7.30-7.20 (m, 2H), | | | |
| | 7.12 (br s, 1H), 4.11 (dt, 3H), 3.95-3.81 (m, 2H), | | | |
| | 3.25 (br t, 2H), 2.52-2.37 (m, 2H) | | | |
| 6 | 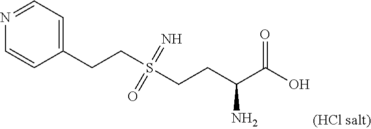 [SMILES: N=S(=O)(CCc1ccncc1)CC[C@H](N)C(=O)O] | 272.0 | 0.317 | M |
| | ¹H NMR (400 MHz, D₂O) δ 8.74 (d, 2H), 8.06 (d, | | | |
| | 2H), 4.13-4.05 (m, 1H), 4.01-3.82 (m, 2H), | | | |
| | 3.78-3.51 (m, 4H), 2.53-2.42 (m, 2H) | | | |
| 7 | 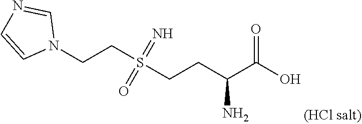 [SMILES: N=S(=O)(CC[C@H](N)C(=O)O)CCn1ccnc1] | 261.0 | 0.251 | A |
| | ¹H NMR (400 MHz, D₂O) δ 8.87 (s, 1H), 7.64- | | | |
| | 7.59 (m, 1H), 7.49 (s, 1H), 4.83 (dt, 2H), 4.13 (dt, | | | |
| | 1H), 4.06-3.87 (m, 2H), 3.65-3.43 (m, 2H), | | | |
| | 2.42 (td, 2H) | | | |
| 8 | 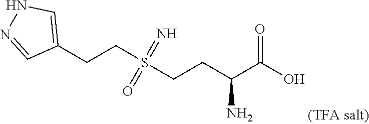 [SMILES: N=S(=O)(CCc1cn[nH]c1)CC[C@H](N)C(=O)O] | 261.0 | 0.265 | C |
| | ¹H NMR (400 MHz, D₂O) δ 7.85 (d, 2H), 4.00- | | | |
| | 3.94 (m, 1H), 3.89-3.79 (m, 2H), 3.78-3.53 (m, | | | |
| | 2H), 3.14 (t, 2H), 2.43-2.29 (m, 2H). | | | |
| 9 | 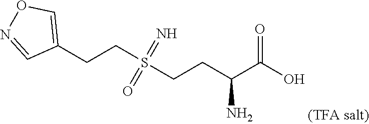 [SMILES: N=S(=O)(CCc1cnoc1)CC[C@H](N)C(=O)O] | 262.0 | 0.273 | A |
| | ¹H NMR (400 MHz, D₂O) δ 8.57 (s, 1H), 8.43 (s, | | | |
| | 1H), 4.14-4.00 (m, 1H), 3.95-3.69 (m, 4H), | | | |
| | 3.12 (br t, 2H), 2.42 (br d, 2H) | | | |
| 10 | 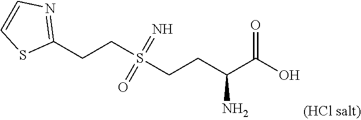 [SMILES: N=S(=O)(CCc1nccs1)CC[C@H](N)C(=O)O] | 278.0 | 0.572 | A |
| | ¹H NMR (400 MHz, D₂O) δ 7.91 (d, 1H), 7.76 (d, | | | |
| | 1H), 4.14 (t, 1H), 4.11-4.00 (m, 2H), 3.83-3.78 | | | |
| | (m, 2H), 3.78-3.65 (m, 2H), 2.50-2.39 (m, 2H). | | | |
| 11 | 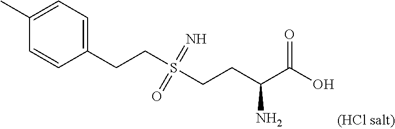 [SMILES: Cc1ccc(CCS(=N)(=O)CC[C@H](N)C(=O)O)cc1] | 285.0 | 1.567 | C |
| | ¹H NMR (400 MHz, CDCl₃-d) δ 7.28-7.18 (m, | | | |
| | 4H), 4.07-3.92 (m, 3H), 3.81-3.60 (m, 2H), | | | |
| | 3.21 (br t, 2H), 2.42-2.33 (m, 2H), 2.29 (s, 3H) | | | |
| 12 | 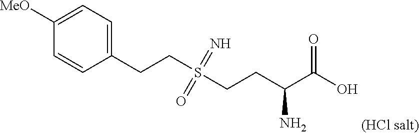 [SMILES: COc1ccc(CCS(=N)(=O)CC[C@H](N)C(=O)O)cc1] | 301.0 | 2.086 | C |
| | ¹H NMR (400 MHz, D₂O) δ 7.26 (d, 2H), 6.94 (d, | | | |
| | 2H), 4.02-3.88 (m, 3H), 3.80-3.74 (m, 3H), | | | |
| | 3.74-3.58 (m, 2H), 3.17 (t, 2H), 2.40-2.25 (m, | | | |
| | 2H). | | | |
| 13 | 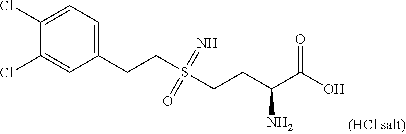 [SMILES: N=S(=O)(CCc1ccc(Cl)c(Cl)c1)CC[C@H](N)C(=O)O] | 339.0 | 1.869 | C |
| | ¹H NMR (400 MHz, DMSO-d₆) δ 7.70 (d, 1H), | | | |
| | 7.61 (d, 1H), 7.38 (dd, 1H), 4.20-4.11 (m, 1H), | | | |
| | 3.89-3.65 (m, 4H), 3.23-3.06 (m, 2H), 2.46- | | | |
| | 2.35 (m, 2H). | | | |
| 14 | 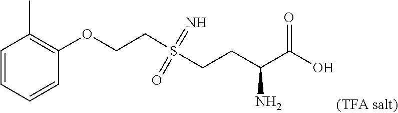 [SMILES: Cc1ccccc1OCCS(=N)(=O)CC[C@H](N)C(=O)O] | 301.0 | 1.541 | C |
| | ¹H NMR (400 MHz, D₂O) δ 7.29-7.17 (m, 2H), | | | |
| | 7.06-6.92 (m, 2H), 4.48 (t, 2H), 3.84 (q, 1H), | | | |
| | 3.79 (t, 2H), 3.67-3.38 (m, 2H), 2.45-2.34 (m, | | | |
| | 2H), 2.20 (s, 3H) | | | |
| 15 | 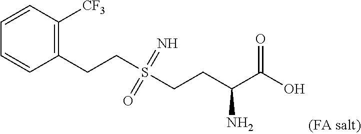 [SMILES: N=S(=O)(CCc1ccccc1C(F)(F)F)CC[C@H](N)C(=O)O] | 339.0 | 1.73 | C |
| | ¹H NMR (400 MHz, D₂O) δ 7.69 (d, 1H), 7.61- | | | |
| | 7.52 (m, 1H), 7.50-7.37 (m, 2H), 3.86-3.77 (m, | | | |
| | 1H), 3.53-3.21 (m, 6H), 2.39-2.23 (m, 2H) | | | |
| 16 | 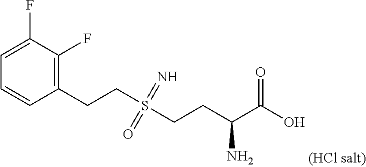 [SMILES: N=S(=O)(CCc1cccc(F)c1F)CC[C@H](N)C(=O)O] | 307.0 | 2.166 | A |
| | ¹H NMR (400 MHz, D₂O) δ = 7.27-7.07 (m, | | | |
| | 3H), 3.94-3.86 (m, 1H), 3.68 (br d, 2H), 3.60- | | | |
| | 3.49 (m, 1H), 3.48-3.39 (m, 1H), 3.24 (br t, 2H), | | | |
| | 2.37 (q, 2H) | | | |
| 17 | 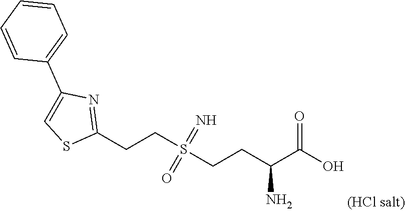 [SMILES: N=S(=O)(CCc1nc(-c2ccccc2)cs1)CC[C@H](N)C(=O)O] | 354.0 | 2.906 | A |
| | ¹H NMR (400 MHz, DMSO-d₆) δ 8.02 (s, 1H), | | | |
| | 7.98-7.92 (m, 2H), 7.79-7.52 (m, 2H), 7.48- | | | |
| | 7.40 (m, 2H), 7.38-7.31 (m, 1H), 4.09-3.98 (m, | | | |
| | 1H), 3.57-3.43 (m, 4H), 3.26-3.14 (m, 2H), | | | |
| | 2.23- 2.02 (m, 2H) | | | |
| 18 | 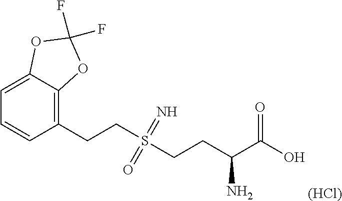 | 351.0 | 1.758 | C |
| | ¹H NMR (400 MHz, D₂O) δ 7.08-6.91 (m, 3H), | | | |
| | 4.07-3.84 (m, 3H), 3.82-3.57 (m, 2H), 3.25 (t, | | | |
| | 2H), 2.44- 2.30 (m, 2H) | | | |
| 19 | 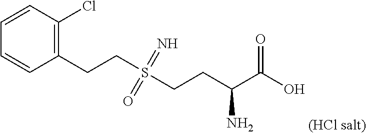 [SMILES: N=S(=O)(CCc1ccccc1Cl)CC[C@H](N)C(=O)O] | 305.0 | 1.795 | N |
| | ¹H NMR (400 MHz, D₂O) δ 7.51-7.44 (m, 1H), | | | |
| | 7.43-7.37 (m, 1H), 7.36-7.29 (m, 2H), 4.07- | | | |
| | 3.94 (m, 3H), 3.89-3.74 (m, 2H), 3.40-3.32 (m, | | | |
| | 2H), 2.48-2.39 (m, 2H) | | | |
| 20 | 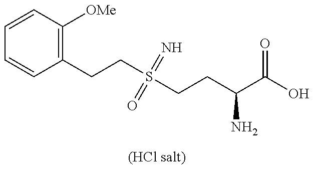 [SMILES: COc1ccccc1CCS(=N)(=O)CC[C@H](N)C(=O)O] | 301.1 | 3.392 | 0 |
| | ¹H NMR (400 MHz, D₂O) δ 7.41-7.20 (m, 2H), | | | |
| | 7.10-6.92 (m, 2H), 3.96-3.91 (m, 2H), 3.86 (s, | | | |
| | 3H), 3.78-3.59 (m, 2H), 3.29-3.12 (m, 2H), | | | |
| | 2.44-2.30 (m, 2H) | | | |
| 21 | 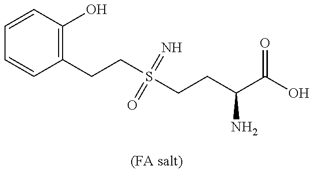 [SMILES: N=S(=O)(CCc1ccccc1O)CC[C@H](N)C(=O)O] | 287.1 | 2.708 | 0 |
| | ¹H NMR (400 MHz, D₂O) δ 7.32-7.12 (m, 2H), | | | |
| | 7.02-6.80 (m, 2H), 3.80 (dt, 1H), 3.62-3.48 (m, | | | |
| | 2H), 3.42-3.21 (m, 2H), 3.10 (t, 2H), 2.43-2.21 | | | |
| | (m, 2H) | | | |
| 22 | 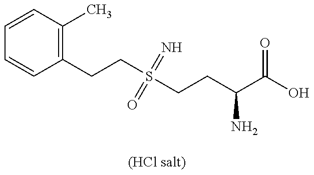 [SMILES: Cc1ccccc1CCS(=N)(=O)CC[C@H](N)C(=O)O] | 285.1 | 1.986 | P |
| | ¹H NMR (400 MHz, D₂O) δ 7.34-7.17 (m, 4H), | | | |
| | 4.09-3.96 (m, 3H), 3.91-3.75 (m, 2H), 3.30- | | | |
| | 3.21 (m, 2H), 2.48-2.37 (m, 2H), 2.32 (s, 3H) | | | |
| 23 | 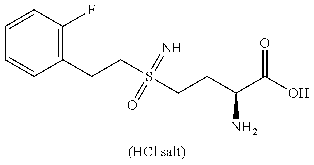 [SMILES: N=S(=O)(CCc1ccccc1F)CC[C@H](N)C(=O)O] | 289.0 | 2.036 | A |
| | ¹H NMR (400 MHz, D₂O) δ 7.44-7.36 (m, 2H), | | | |
| | 7.25-7.17 (m, 2H), 4.13-4.01 (m, 3H), 3.94- | | | |
| | 3.73 (m, 2H), 3.33 (t, 2H), 2.52-2.43 (m, 2H) | | | |
| 24 | 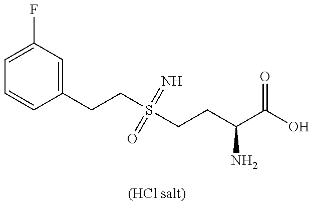 [SMILES: N=S(=O)(CCc1cccc(F)c1)CC[C@H](N)C(=O)O] | 289.0 | 1.138 | Q |
| | ¹H NMR (400 MHz, D₂O) δ 7.46-7.33 (m, 1H), | | | |
| | 7.21-7.01 (m, 3H), 3.90-3.79 (m, 1H), 3.67- | | | |
| | 3.56 (m, 2H), 3.47-3.24 (m, 2H), 3.18 (t, 2H), | | | |
| | 2.41-2.29 (m, 2H) | | | |
| 25 | 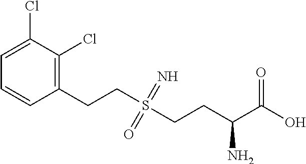 [SMILES: N=S(=O)(CCc1cccc(Cl)c1Cl)CC[C@H](N)C(=O)O] | 339.1 (M+) | 2.931 | A |
| | ¹H NMR (400 MHz, D₂O) δ = 7.50 (d, 1H), 7.37- | | | |
| | 7.33 (m, 1H), 7.31-7.25 (m, 1H), 3.95-3.89 | | | |
| | (m, 1H), 3.72-3.66 (m, 2H), 3.62-3.45 (m, 2H), | | | |
| | 3.36-3.31 (m, 2H), 2.38 (q, 2H) | | | |
| 26 | 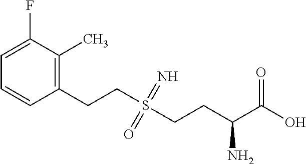 [SMILES: Cc1c(F)cccc1CCS(=N)(=O)CC[C@H](N)C(=O)O] | 303.1 | 1.778 | B |
| | ¹H NMR (400 MHz, D₂O) δ 7.23-7.16 (m, 1H), | | | |
| | 7.09 (d, 1H), 7.06-7.00 (m, 1H), 3.99 (dt, 1H), | | | |
| | 3.89-3.82 (m, 2H), 3.77-3.60 (m, 2H), 3.25 (t, | | | |
| | 2H), 2.44-2.36 (m, 2H), 2.22 (d, 3H) | | | |
| 27 | 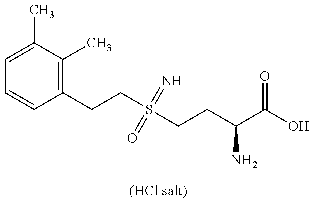 [SMILES: Cc1cccc(CCS(=N)(=O)CC[C@H](N)C(=O)O)c1C] | 299.0 | 2.462 | H |
| | ¹H NMR (400 MHz, DMSO-d₆) δ = 7.74-7.48 | | | |
| | (m, 1H), 7.18-6.82 (m, 3H), 4.01-3.87 (m, 1H), | | | |
| | 3.20-3.09 (m, 3H), 3.09-2.84 (m, 3H), 2.23 (s, | | | |
| | 3H), 2.18 (s, 3H), 2.16 -2.09 (m, 1H), 2.09-1.98 | | | |
| | (m, 1H) | | | |
| 28 | 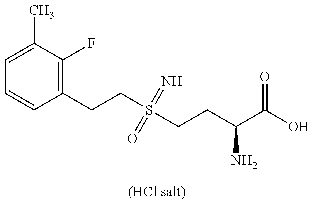 [SMILES: Cc1cccc(CCS(=N)(=O)CC[C@H](N)C(=O)O)c1F] | 323.0 | 2.553 | A |
| | ¹H NMR (400 MHz, D₂O) δ 7.47-7.41 (m, 1H), | | | |
| | 7.30 (br t, 1H), 7.18-7.11 (m, 1H), 4.11 (br t, | | | |
| | 1H), 4.03-3.94 (m, 2H), 3.88-3.77 (m, 2H), | | | |
| | 3.32 (br t, 2H), 2.51-2.39 (m, 2H) | | | |
| 29 | 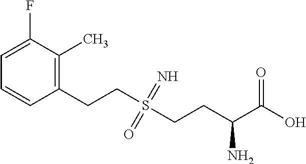 [SMILES: Cc1c(F)cccc1CCS(=N)(=O)CC[C@H](N)C(=O)O] | 357.0 | 1.704 | C |
| | ¹H NMR (400 MHz, D₂O) δ 7.55 (dt, 1H), 7.28- | | | |
| | 7.15 (m, 2H), 3.85-3.76 (m, 1H), 3.53-3.40 (m, | | | |
| | 3H), 3.36-3.26 (m, 3H), 2.38-2.26 (m, 2H) | | | |
| 30 | 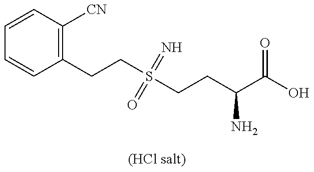 [SMILES: N#Cc1ccccc1CCS(=N)(=O)CC[C@H](N)C(=O)O] | 296.2 | 2.858 | 0 |
| | ¹H NMR (400 MHz, D₂O) δ 7.78 (d, 1H), 7.71- | | | |
| | 7.65 (m, 1H), 7.54 (d, 1H), 7.49-7.44 (m, 1H), | | | |
| | 3.89-3.83 (m, 1H), 3.67-3.61 (m, 2H), 3.52- | | | |
| | 3.41 (m, 1H), 3.40-3.35 (m, 3H), 2.37 (q, 2H) | | | |
| 31 | 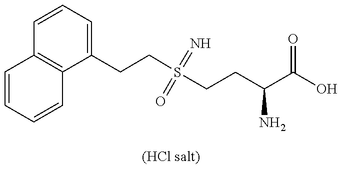 [SMILES: N=S(=O)(CCc1cccc2ccccc12)CC[C@H](N)C(=O)O] | 321.2 | 1.905 | N |
| | ¹H NMR (400 MHz, D₂O) δ 8.14 (d, 1H), 8.07- | | | |
| | 8.03 (m, 1H), 7.99-7.95 (m, 1H), 7.73-7.62 (m, | | | |
| | 2H), 7.61-7.54 (m, 2H), 4.00-3.90 (m, 3H), | | | |
| | 3.78-3.72 (m, 2H), 2.41-2.34 (m, 2H) | | | |
| 32 | 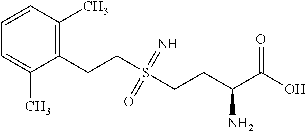 [SMILES: Cc1cccc(C)c1CCS(=N)(=O)CC[C@H](N)C(=O)O] | 299.1 | 1.835 | N |
| | ¹H NMR (400 MHz, D₂O) δ 7.15-7.02 (m, 3H), | | | |
| | 4.21 (dd, 1H), 4.07 (t, 2H), 3.98-3.89 (m, 2H), | | | |
| | 3.32-3.23 (m, 2H), 2.57-2.47 (m, 2H), 2.29 (s, | | | |
| | 6H) | | | |
| 33 | 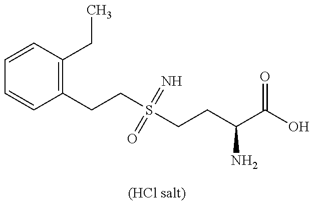 [SMILES: CCc1ccccc1CCS(=N)(=O)CC[C@H](N)C(=O)O] | 299.2 | 1.84 | N |
| | ¹H NMR (400 MHz, D₂O) δ 7.48-7.23 (m, 4H), | | | |
| | 3.92 (br t, 1H), 3.63-3.48 (m, 3H), 3.46-3.35 | | | |
| | (m, 1H), 3.26 (br t, 2H), 2.77 (q, 2H), 2.49-2.38 | | | |
| | (m, 2H), 1.28 (t, 3H) | | | |
| 34 | 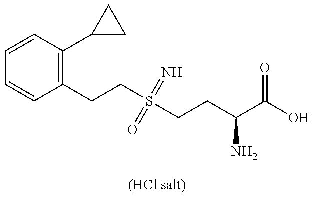 [SMILES: N=S(=O)(CCc1ccccc1C1CC1)CC[C@H](N)C(=O)O] | 311.2 | 1.871 | N |
| | ¹H NMR (400 MHz, D₂O) δ 7.31-7.18 (m, 3H), | | | |
| | 7.09 (d, 1H), 3.95-3.82 (m, 3H), 3.58 (br s, 2H), | | | |
| | 3.45-3.38 (m, 2H), 2.43-2.34 (m, 2H), 2.00- | | | |
| | 1.91 (m, 1H), 0.99 (br dd, 2H), 0.70-0.63 (m, | | | |
| | 2H) | | | |
| 35 | 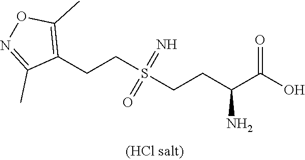 [SMILES: Cc1noc(C)c1CCS(=N)(=O)CC[C@H](N)C(=O)O] | 290.0 | 0.983 | E |
| | ¹H NMR (400 MHz, D₂O) δ 3.92-3.78 (m, 1H), | | | |
| | 3.50-3.24 (m, 4H), 2.98-2.82 (m, 2H), 2.41- | | | |
| | 2.27 (m, 5H), 2.22 (s, 3H) | | | |
| 36 | 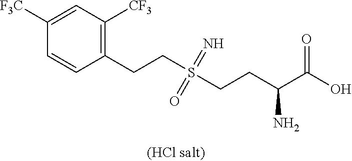 [SMILES: N=S(=O)(CCc1ccc(C(F)(F)F)cc1C(F)(F)F)CC[C@H](N)C(=O)O] | 407.2 | 2.127 | N |
| | ¹H NMR (400 MHz, D₂O) δ = 7.94 (d, 1H), 7.82- | | | |
| | 7.76 (m, 1H), 7.58 (dd, 1H), 4.17 (dd, 1H), 4.13- | | | |
| | 3.97 (m, 4H), 3.38 (s, 2H), 2.53-2.38 (m, 2H) | | | |
| 37 | 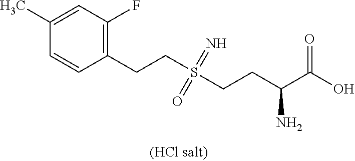 [SMILES: Cc1ccc(CCS(=N)(=O)CC[C@H](N)C(=O)O)c(F)c1] | 303.2 | 1.765 | N |
| | ¹H NMR (400 MHz, D₂O) δ 7.23 (t, 1H), 7.03- | | | |
| | 6.98 (m, 2H), 4.14-4.11 (m, 1H), 4.11-3.99 (m, | | | |
| | 3H), 3.95-3.76 (m, 2H), 3.24 (t, 2H), 2.49-2.37 | | | |
| | (m, 2H), 2.29 (s, 3H) | | | |
| 38 | 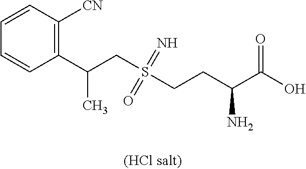 [SMILES: CC(CS(=N)(=O)CC[C@H](N)C(=O)O)c1ccccc1C#N] | 319.1 | 1.805 | N |
| | ¹H NMR (400 MHz, D₂O) δ 7.53-7.45 (m, 2H), | | | |
| | 7.40-7.35 (m, 1H), 7.33-7.27 (m, 1H), 4.32- | | | |
| | 4.17 (m, 1H), 4.11-3.94 (m, 3H), 3.75-3.50 (m, | | | |
| | 2H), 2.43-2.27 (m, 2H), 1.44 (d, 3H) | | | |
| 39 | 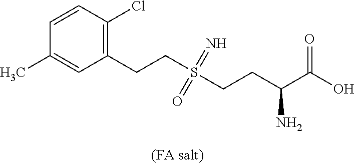 [SMILES: Cc1ccc(Cl)c(CCS(=N)(=O)CC[C@H](N)C(=O)O)c1] | 319.1 | 1.872 | N |
| | ¹H NMR (400 MHz, DMSO-d₆ + MeOD-d₄) δ | | | |
| | 7.32-7.19 (m, 2H), 7.06 (dd, 1H), 3.45 (t, 1H), | | | |
| | 3.39-3.27 (m, 1H), 3.26-3.19 (m, 2H), 3.13- | | | |
| | 3.02 (m, 2H), 2.26 (s, 3H), 2.24-2.05 (m, 2H) | | | |
| 40 | 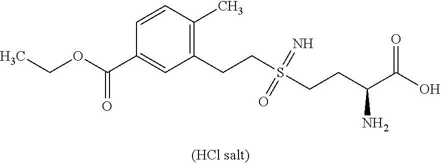 [SMILES: CCOC(=O)c1ccc(C)c(CCS(=N)(=O)CC[C@H](N)C(=O)O)c1] | 357.3 | 1.932 | N |
| | ¹H NMR (400 MHz, D₂O) δ 7.90-7.87 (m, 1H), | | | |
| | 7.82 (dd, 1H), 7.35 (d, 1H), 4.35 (q, 2H), 3.87- | | | |
| | 3.79 (m, 1H), 3.55-3.33 (m, 4H), 3.23-3.16 (m, | | | |
| | 2H), 2.40-2.30 (m, 5H), 1.35 (t, 3H) | | | |
| 41 | 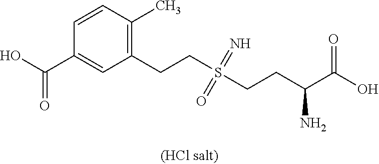 [SMILES: Cc1ccc(C(=O)O)cc1CCS(=N)(=O)CC[C@H](N)C(=O)O] | 329.2 | 1.559 | N |
| | ¹H NMR (400 MHz, D₂O) δ 7.85 (d, 1H), 7.81- | | | |
| | 7.77 (m, 1H), 7.35 (d, 1H), 3.85 (dt, 1H), 3.58- | | | |
| | 3.53 (m, 2H), 3.49-3.34 (m, 2H), 3.22-3.16 (m, | | | |
| | 2H), 2.39 (s, 3H), 2.37-2.32 (m, 2H) | | | |
| 42 | 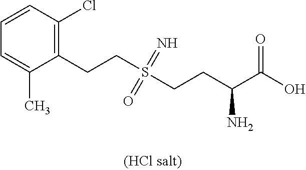 [SMILES: Cc1cccc(Cl)c1CCS(=N)(=O)CC[C@H](N)C(=O)O] | 319.0 | 2.768 | A |
| | ¹H NMR (400 MHz, D₂O) δ 7.34-7.26 (m, 1H), | | | |
| | 7.18 (d, 2H), 4.22 (dd, 1H), 4.15-3.98 (m, 4H), | | | |
| | 3.45-3.35 (m, 2H), 2.61-2.48 (m, 2H), 2.37- | | | |
| | 2.32 (m, 3H) | | | |
| 43 | 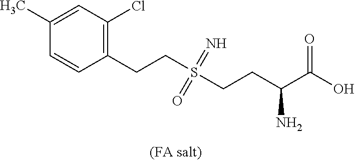 [SMILES: Cc1ccc(CCS(=N)(=O)CC[C@H](N)C(=O)O)c(Cl)c1] | 319.1 | 2.76 | A |
| | ¹H NMR (400 MHz, DMSO-d₆) δ 2.01-2.21 (m, | | | |
| | 2 H) 2.28 (s, 3 H) 3.01-3.10 (m, 2 H) 3.16 (s, 2 | | | |
| | H) 3.18-3.22 (m, 2 H) 3.23-3.25 (m, 1 H) 7.12 | | | |
| | (d, 1 H) 7.27-7.33 (m, 2 H) | | | |
| 44 | 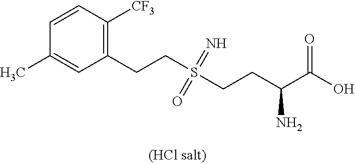 [SMILES: Cc1ccc(C(F)(F)F)c(CCS(=N)(=O)CC[C@H](N)C(=O)O)c1] | 353.0 | 2.945 | H |
| | ¹H NMR (400 MHz, D₂O) δ 7.59 (d, 1H), 7.36- | | | |
| | 7.19 (m, 2H), 4.01 (t, 1H), 3.86-3.60 (m, 4H), | | | |
| | 3.29 (br t, 2H), 2.41 (q, 2H), 2.33 (s, 3H) | | | |
| 45 | 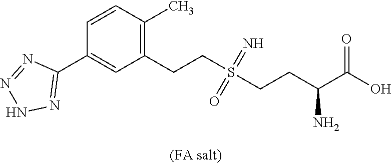 [SMILES: Cc1ccc(-c2nn[nH]n2)cc1CCS(=N)(=O)CC[C@H](N)C(=O)O] | 353.1 | 2.151 | A |
| | ¹H NMR (400 MHz, D₂O) δ 7.78-7.67 (m, 2H), | | | |
| | 7.38 (d, H), 3.85 (dt, 1H), 3.61-3.30 (m, 4H), | | | |
| | 3.23-3.15 (m, 2H), 2.41-2.30 (m, 5H) | | | |
| 46 | 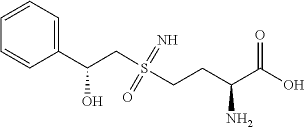 [SMILES: N=S(=O)(CC[C@H](N)C(=O)O)C[C@H](O)c1ccccc1] | 287.0 | 0.675 | D |
| | ¹H NMR (400 MHz, D₂O) δ 7.48-7.43 (m, 4H), | | | |
| | 7.43-7.36 (m, 1H), 5.28 (dd, 1H), 3.91-3.76 | | | |
| | (m, 2H), 3.61-3.48 (m, 2H), 3.48-3.31 (m, 1H), | | | |
| | 2.49-2.29 (m, 2H) | | | |
| 47 | 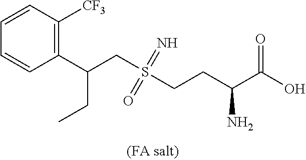 [SMILES: CCC(CS(=N)(=O)CC[C@H](N)C(=O)O)c1ccccc1C(F)(F)F] | 367.1 | 3.301 | A |
| | ¹H NMR (400 MHz, D₂O) δ 7.75 (br d, 1H), 7.68- | | | |
| | 7.59 (m, 2H), 7.47-7.41 (m, 1H), 3.78-3.58 | | | |
| | (m, 4H), 3.35-3.13 (m, 2H), 2.35-2.13 (m, 2H), | | | |
| | 1.90 (qd, 1H), 1.77 (qd, 1H), 0.86-0.72 (m, 3H) | | | |
| 48 | 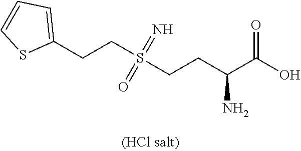 [SMILES: N=S(=O)(CCc1cccs1)CC[C@H](N)C(=O)O] | 277.0 | 1.425 | E |
| | ¹H NMR (400 MHz, D₂O) δ 7.28 (dd, 1H), 7.02- | | | |
| | 6.91 (m, 2H), 3.88-3.72 (m, 3H), 3.54-3.34 (m, | | | |
| | 4H), 2.35-2.20 (m, 2H) | | | |

### Exemplary Embodiment 1c

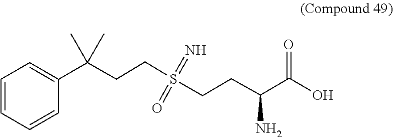 [SMILES: CC(C)(CCS(=N)(=O)CC[C@H](N)C(=O)O)c1ccccc1]

### (2S)-2-amino-4-(3-methyl-3-phenylbutylsulfonimidoyl)butanoic acid

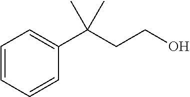

To a solution of 3-methyl-3-phenylbutanoic acid (1.00 g, 5.61 mmol) in THE (12 mL) was added LiAlH₄ (213 mg, 5.61 mmol) at 0° C. The mixture was stirred at 20° C. for 2 h under N₂. The mixture was diluted with ethyl acetate (10 mL), quenched with water (0.2 mL), 15% NaOH (0.2 mL), and water (0.6 mL). The mixture was dried over Na₂SO₄, filtered, and the filtrate was concentrated. The residue was purified by prep-TLC (Petroleum ether: ethyl acetate=5:1) to give 3-methyl-3-phenylbutan-1-ol (640 mg, 6900 yield) as a colorless oil. ¹H NM/R (400 MHz, CDCl₃-d) δ 7.40-7.29 (m, 4H), 7.23-7.17 (m, 1H), 3.57-3.46 (m, 2H), 2.02-1.93 (m, 2H), 1.36 (s, 6H).

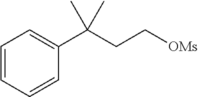 [SMILES: CC(C)(CCOS(C)(=O)=O)c1ccccc1]

To a solution of 3-methyl-3-phenylbutan-1-ol (640 mg, 3.90 mmol) and TEA (592 mg, 5.84 mmol, 813 mL) in DCM (6 mL) was added MsCl (536 mg, 4.68 mmol, 362 mL) at 0° C. The mixture was stirred at 20° C. for 1 h. The mixture was poured into water (10 mL) and extracted with EtOAc (15 mL×2). The combined organic extracts were dried over Na₂SO₄, filtered, and concentrated. The residue was purified by column chromatography (SiO₂, Petroleum ether: ethyl acetate=92:8) to give 3-methyl-3-phenylbutyl methanesulfonate (880 mg, 93% yield) as a colorless oil. ¹H NMR (400 MHz, CDCl₃-d) δ 7.37-7.31 (m, 4H), 7.25-7.18 (m, 1H), 4.09-3.98 (m, 2H), 2.89-2.81 (m, 3H), 2.21-2.07 (m, 2H), 1.39 (s, 6H).

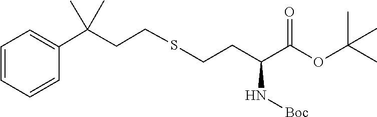 [SMILES: CC(C)(C)OC(=O)N[C@@H](CCSCCC(C)(C)c1ccccc1)C(=O)OC(C)(C)C]

To a solution of (S)-tert-butyl 2-((tert-butoxycarbonyl)amino)-4-mercaptobutanoate (880 mg, 3.02 mmol) in DMF (8 mL) was added 3-methyl-3-phenylbutyl methanesulfonate (879 mg, 3.62 mmol), K₂CO₃ (1.25 g, 9.06 mmol) and KI (1.0 g, 6.04 mmol). The mixture was stirred at 25° C. for 16 h under Ar. The mixture was poured into water (10 mL) and extracted with EtOAc (15 mL×3). The combined organic extracts were dried over Na₂SO₄, filtered, and concentrated. The residue was purified by prep-TLC (SiO₂, Petroleum ether: ethyl acetate=5:1) to give (S)-tert-butyl 2-((tert-butoxycarbonyl)amino)-4-((3-methyl-3-phenylbutyl)thio)butanoate (700 mg, 53% yield) as a colorless oil. ¹H NMR (400 MHz, CDCl₃-d) δ 7.32 (d, J=4.4 Hz, 4H), 7.23-7.16 (m, 1H), 5.05 (br d, J=7.2 Hz, 1H), 4.22 (br d, J=5.3 Hz, 1H), 2.53-2.39 (m, 2H), 2.28-2.17 (m, 2H), 1.98 (br s, 1H), 1.94-1.86 (m, 2H), 1.82-1.70 (m, 1H), 1.50-1.42 (m, 18H), 1.33 (s, 6H).

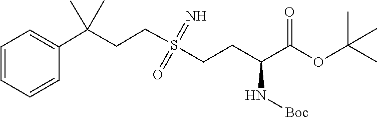 [SMILES: CC(C)(C)OC(=O)N[C@@H](CCS(=N)(=O)CCC(C)(C)c1ccccc1)C(=O)OC(C)(C)C]

To a solution of (S)-tert-butyl 2-((tert-butoxycarbonyl)amino)-4-((3-methyl-3-phenylbutyl)thio)butanoate (500 mg, 1.14 mmol) in i-PrOH (5 mL) was added PhI(OAc)₂ (1.47 g, 4.57 mmol) and ammonium carbamate (714 mg, 9.14 mmol). The mixture was stirred at 20° C. for 16 h. The mixture was poured into water (10 mL) and extracted with EtOAc (10 mL×3). The combined organic extracts were dried over Na₂SO₄, filtered, and concentrated. The residue was purified by column chromatography (SiO₂, Petroleum ether: ethyl acetate=70:30) to give (2S)-tert-butyl 2-((tert-butoxycarbonyl)amino)-4-(3-methyl-3-phenylbutylsulfonimidoyl)butanoate (400 mg, 75% yield) as a colorless oil. ¹H NMR (400 MHz, CDCl₃-d) δ 7.38-7.30 (m, 4H), 7.25-7.19 (m, 1H), 5.19 (br s, 1H), 4.23 (br s, 1H), 3.16-2.90 (m, 2H), 2.89-2.66 (m, 2H), 2.36-2.22 (m, 1H), 2.20-2.09 (m, 2H), 2.05-1.92 (m, 1H), 1.46 (d, J=7.5 Hz, 18H), 1.39 (s, 6H).

A solution of (2S)-tert-butyl 2-((tert-butoxycarbonyl)amino)-4-(3-methyl-3-phenylbutylsulfonimidoyl)butanoate (70.0 mg, 149 mmol) in HCl/dioxane (2 mL, 4 M) was stirred at 25° C. for 2 h. The reaction mixture was concentrated and the residue was dissolved in water (2 mL) and extracted with DCM (1 mL×2). The water layer was lyophilized to give (2S)-2-amino-4-(3-methyl-3-phenylbutylsulfonimidoyl)butanoic acid (36.0 mg, 69% yield, HCl salt) as a yellow solid. LCMS: Rt=2.776 min, (ES⁺) m/z (M+H)⁺=313.1, HPLC Conditions: A; ¹H NMR (400 MHz, D₂O) δ 7.51-7.37 (m, 4H), 7.34-7.25 (m, 1H), 4.10-3.97 (m, 1H), 3.96-3.73 (m, 2H), 3.43 (br d, J=7.6 Hz, 2H), 2.35 (br d, J=6.7 Hz, 2H), 2.27-2.15 (m, 2H), 1.38 (s, 6H).

### Exemplary Embodiment 1d

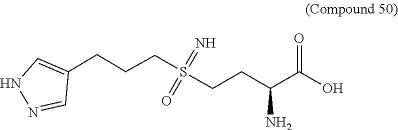 [SMILES: N=S(=O)(CCCc1cn[nH]c1)CC[C@H](N)C(=O)O]

### (2S)-4-(3-(1H-pyrazol-4-yl)propylsulfonimidoyl)-2-aminobutanoic acid

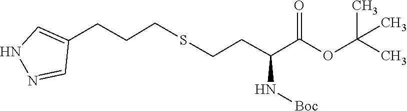 [SMILES: CC(C)(C)OC(=O)N[C@@H](CCSCCCc1cn[nH]c1)C(=O)OC(C)(C)C]

To a solution of (S)-tert-butyl 2-((tert-butoxycarbonyl)amino)-4-mercaptobutanoate (500 mg, 1.72 mmol) and 4-(3-chloropropyl)-1H-pyrazole (372 mg, 2.06 mmol, HCl) in DMF (5 mL) was added K₂CO₃ (711 mg, 5.15 mmol) and KI (569 mg, 3.43 mmol). The mixture was stirred at 20° C. for 16 h under Ar. The mixture was poured into water (10 mL) and extracted with EtOAc (10 mL×3). The combined organic extracts were dried over Na₂SO₄, filtered, and concentrated. The residue was purified by column chromatography (SiO₂, Petroleum ether: ethyl acetate=75:25 to 60:40) to afford (S)-tert-butyl 4-((3-(1H-pyrazol-4-yl)propyl)thio)-2-((tert-butoxycarbonyl)amino)butanoate (600 mg, 88% yield) as a yellow oil. ¹H NMR (400 MHz, CDCl₃-d) δ 7.50-7.40 (m, 3H), 5.11 (br d, J=6.7 Hz, 1H), 4.28 (br s, 1H), 2.63 (t, J=7.4 Hz, 2H), 2.58-2.48 (m, 4H), 2.07-2.00 (m, 1H), 1.93-1.82 (m, 3H), 1.46 (d, J=8.3 Hz, 18H).

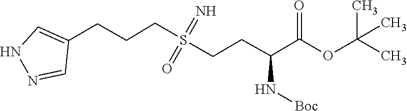 [SMILES: CC(C)(C)OC(=O)N[C@@H](CCS(=N)(=O)CCCc1cn[nH]c1)C(=O)OC(C)(C)C]

To a solution of (S)-tert-butyl 4-((3-(1H-pyrazol-4-yl)propyl)thio)-2-((tert-butoxycarbonyl)amino)butanoate (200 mg, 0.5 mmol) in i-PrOH (2 mL) was added PhI(OAc)₂ (644 mg, 2.0 mmol) and ammonium carbamate (312 mg, 4.0 mmol). The mixture was stirred at 20° C. for 16 h. The reaction mixture was concentrated. The mixture was poured into water (1 mL) and extracted with EtOAc (2 mL×3). The combined organic extracts were dried over Na₂SO₄, filtered, and concentrated. The residue was purified by prep-HPLC (column: Phenomenex Gemini-NX C18 75×30 mm×3 um; mobile phase: [water (0.2% FA)-MeCN]; B %: 10%-40%, 12 min) to give (2S)-tert-butyl 4-(3-(1H-pyrazol-4-yl)propylsulfonimidoyl)-2-((tert-butoxycarbonyl)amino)butanoate (70 mg, 33% yield) as a white solid. ¹H NMR (400 MHz, CDCl₃-d) δ 7.43 (s, 2H), 5.39 (br d, J=6.8 Hz, 1H), 4.26 (br s, 1H), 3.23-2.96 (m, 4H), 2.68 (t, J=7.3 Hz, 2H), 2.46-2.28 (m, 1H), 2.21-1.99 (m, 3H), 1.45 (d, J=12.5 Hz, 18H).

To a solution of (2S)-tert-butyl 4-(3-(1H-pyrazol-4-yl)propylsulfonimidoyl)-2-((tert-butoxycarbonyl)amino)butanoate (30 mg, 70 mmol) in HCl/dioxane (1 mL) mixture was stirred at 20° C. for 1 h. The reaction mixture was concentrated to give (2S)-4-(3-(1H-pyrazol-4-yl)propylsulfonimidoyl)-2-aminobutanoic acid (19 mg, 87% yield, HCl) as a colorless solid. LCMS: Rt=0.398 min., (ES⁺) m/z (M+H)⁺=275.0, HPLC Conditions: A; ¹H NMR (400 MHz, D₂O) δ 7.94 (s, 2H), 4.11-3.96 (m, 1H), 3.93-3.55 (m, 4H), 2.87-2.67 (m, 2H), 2.50-2.35 (m, 2H), 2.17 (q, J=7.8 Hz, 2H).

The compounds described in Table 6 were prepared using the methods outlined above. “HPLC rt” designates retention time in an HPLC analytical experiment consistent with purification methods described above.

TABLE 6
Characterization data for compounds 51-66

| Compound | | MS | HPLC | HPLC |
|---|---|---|---|---|
| # | Structure | (M + H)+ | rt | cond. |
| 51 | 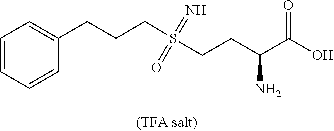 [SMILES: N=S(=O)(CCCc1ccccc1)CC[C@H](N)C(=O)O] | 285.1 | 2.167 | A |
| | ¹H NMR (400 MHz, D₂O) δ 7.45-7.38 (m, 2 H), | | | |
| | 7.38-7.28 (m, 3 H), 4.06-3.97 (m, 1 H), 3.92- | | | |
| | 3.68 (m, 2 H), 3.68-3.52 (m, 2 H), 2.86 (br t, 2 | | | |
| | H), 2.48-2.32 (m, 2 H), 2.24 (quin, 2 H). | | | |
| 52 | 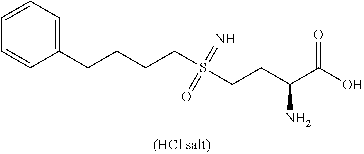 [SMILES: N=S(=O)(CCCCc1ccccc1)CC[C@H](N)C(=O)O] | 299.0 | 1.697 | C |
| | ¹H NMR (400 MHz, D₂O) δ 7.38 - 7.31 (m, 2H), | | | |
| | 7.30-7.22 (m, 3H), 4.00 (dt, 1H), 3.95-3.61 (m, | | | |
| | 4H), 2.69 (t, 2H), 2.49-2.35 (m, 2H), 1.95-1.73 | | | |
| | (m, 4H) | | | |
| 53 | 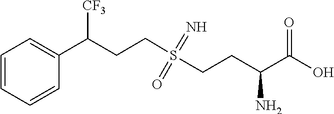 [SMILES: N=S(=O)(CCC(c1ccccc1)C(F)(F)F)CC[C@H](N)C(=O)O] | 353.0 | 1.817 | C |
| | ¹H NMR (400 MHz, D₂O) δ 7.48-7.41 (m, 5H), | | | |
| | 3.95 (s, 1H), 3.77-3.42 (m, 4H), 3.27-3.12 (m, | | | |
| | 1H), 2.64-2.41 (m, 2H), 2.32 (br s, 2H) | | | |
| 54 | 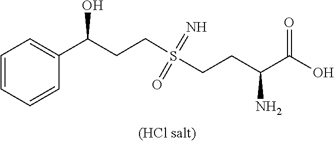 [SMILES: N=S(=O)(CC[C@H](N)C(=O)O)CC[C@H](O)c1ccccc1] | 301.0 | 1.527 | A |
| | ¹H NMR (400 MHz, D₂O) δ 7.53-7.29 (m, 5H), | | | |
| | 4.94-4.84 (m, 1H), 4.16-4.04 (m, 1H), 4.01- | | | |
| | 3.60 (m, 4H), 2.50-2.38 (m, 2H), 2.38-2.27 (m, | | | |
| | 2H) | | | |
| 55 | 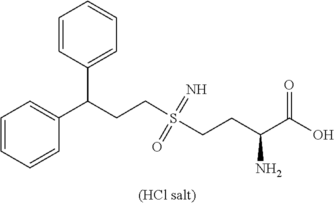 [SMILES: N=S(=O)(CCC(c1ccccc1)c1ccccc1)CC[C@H](N)C(=O)O] | 361.0 | 3.329 | A |
| | ¹H NMR (400 MHz, D₂O) δ = 7.43-7.08 (m, | | | |
| | 10H), 4.19-4.07 (m, 2H), 4.06-3.82 (m, 2H), | | | |
| | 3.70-3.59 (m, 2H), 2.70-2.56 (m, 2H), 2.52- | | | |
| | 2.34 (m, 2H) | | | |
| 56 | 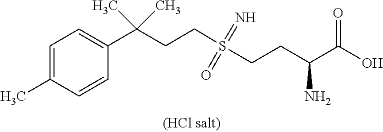 [SMILES: Cc1ccc(C(C)(C)CCS(=N)(=O)CC[C@H](N)C(=O)O)cc1] | 327.1 | 2.102 | N |
| | ¹H NMR (400 MHz, D₂O) δ 7.35 (d, 2H), 7.25 (d, | | | |
| | 2H), 3.92-3.86 (m, 1H), 3.65-3.47 (m, 2H), | | | |
| | 3.25-3.17 (m, 2H), 2.31-2.27 (m, 5H), 2.18- | | | |
| | 2.12 (m, 2H), 1.34 (s, 6H) | | | |
| 57 | 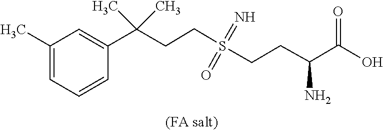 [SMILES: Cc1cccc(C(C)(C)CCS(=N)(=O)CC[C@H](N)C(=O)O)c1] | 327.1 | 3.18 | E |
| | ¹H NMR (400 MHz, D₂O) δ 7.34-7.17 (m, 3H), | | | |
| | 7.08 (d, 1H), 3.80-3.70 (m, 1H), 3.36-3.11 (m, | | | |
| | 2H), 2.97-2.85 (m, 2H), 2.28 (s, 3H), 2.18 (td, | | | |
| | 2H), 2.10-2.01 (m, 2H), 1.30 (s, 6H) | | | |
| 58 | 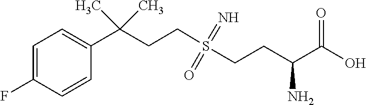 [SMILES: CC(C)(CCS(=N)(=O)CC[C@H](N)C(=O)O)c1ccc(F)cc1] | 331.0 | 2.979 | A |
| | ¹H NMR (400 MHz, D₂O) δ 7.40 (dd, 2H), 7.09 | | | |
| | (t, 2H), 3.86-3.70 (m, 1H), 3.44-3.09 (m, 2H), | | | |
| | 3.00-2.84 (m, 2H), 2.29-2.15 (m, 2H), 2.11- | | | |
| | 2.03 (m, 2H), 1.32 (s, 5H), 1.33-1.29 (m, 1H) | | | |
| 59 | 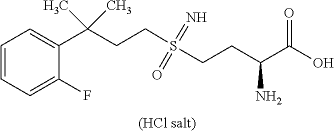 [SMILES: CC(C)(CCS(=N)(=O)CC[C@H](N)C(=O)O)c1ccccc1F] | 331.1 | 2.52 | P |
| | ¹H NMR (400 MHz, D₂O) δ 7.40-7.24 (m, 2H), | | | |
| | 7.20-7.03 (m, 2H), 3.78 (dt, 1H), 3.39-3.14 (m, | | | |
| | 2H), 2.98-2.88 (m, 2H), 2.31-2.19 (m, 4H), | | | |
| | 1.38 (s, 6H). | | | |
| 60 | 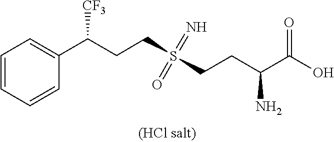 [SMILES: N=[S@](=O)(CC[C@H](c1ccccc1)C(F)(F)F)CC[C@H](N)C(=O)O] | 353.2 | 1.455 | N |
| | ¹H NMR (400 MHz, DMSO-d₆) δ 7.46-7.33 (m, | | | |
| | 5H), 3.87-3.77 (m, 1H), 3.37 (t, 1H), 3.29-3.18 | | | |
| | (m, 1H), 3.12-2.92 (m, 2H), 2.65-2.56 (m, 1H), | | | |
| | 2.42-2.30 (m, 2H), 2.15-1.94 (m, 2H). | | | |
| 61 | 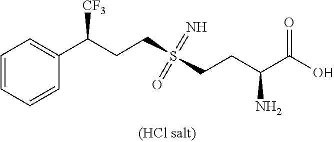 [SMILES: N=[S@@](=O)(CC[C@H](N)C(=O)O)CC[C@@H](c1ccccc1)C(F)(F)F] | 353.1 | 1.919 | N |
| | ¹H NMR (400 MHz, DMSO-d₆) δ 7.50-7.31 (m, | | | |
| | 5H), 3.91-3.78 (m, 1H), 3.38 (br t, 1H), 3.29- | | | |
| | 3.17 (m, 1H), 3.12-3.04 (m, 1H), 3.02-2.90 (m, | | | |
| | 1H), 2.68-2.58 (m, 1H), 2.44-2.37 (m, 1H), | | | |
| | 2.35-2.20 (m, 1H), 2.17-1.94 (m, 2H). | | | |
| 62 | 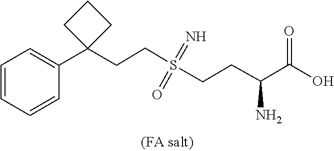 [SMILES: N=S(=O)(CC[C@H](N)C(=O)O)CCC1(c2ccccc2)CCC1] | 325.1 | 2.986 | A |
| | ¹H NMR (400 MHz, D₂O) δ 7.42-7.36 (m, 2H), | | | |
| | 7.28-7.22 (m, 3H), 3.80-3.73 (m, 1H), 3.35- | | | |
| | 3.14 (m, 2H), 2.96-2.87 (m, 2H), 2.42-2.32 (m, | | | |
| | 2H), 2.31-2.24 (m, 2H), 2.22-2.04 (m, 5H), | | | |
| | 1.88-1.77 (m, 1H) | | | |
| 63 | 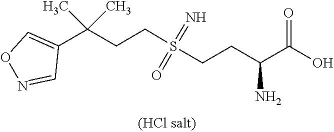 [SMILES: CC(C)(CCS(=N)(=O)CC[C@H](N)C(=O)O)c1cnoc1] | 304.1 | 0.84 | C |
| | ¹H NMR (400 MHz, D₂O) δ 8.44 (s, 2H), 3.82- | | | |
| | 3.71 (m, 1H), 3.38-3.19 (m, 2H), 3.09-3.02 (m, | | | |
| | 2H), 2.23 (q, 2H), 2.00-1.93 (m, 2H), 1.26 (s, | | | |
| | 6H) | | | |
| 64 | 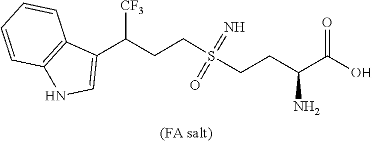 [SMILES: N=S(=O)(CCC(c1c[nH]c2ccccc12)C(F)(F)F)CC[C@H](N)C(=O)O] | 392.0 | 3.03 | E |
| | ¹H NMR (400 MHz, D₂O) δ 7.72 (br d, 1H), 7.63- | | | |
| | 7.40 (m, 2H), 7.37-7.10 (m, 2H), 4.16-3.92 | | | |
| | (m, 1H), 3.82-3.65 (m, 1H), 3.44-3.15 (m, 3H), | | | |
| | 3.09-2.93 (m, 1H), 2.69-2.44 (m, 2H), 2.32- | | | |
| | 2.07 (m, 2H) | | | |
| 65 | 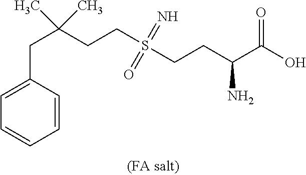 [SMILES: CC(C)(CCS(=N)(=O)CC[C@H](N)C(=O)O)Cc1ccccc1] | 327.1 | 3.257 | A |
| | ¹H NMR (400 MHz, D₂O) δ = 7.37-7.31 (m, | | | |
| | 2H), 7.31-7.19 (m, 3H), 3.90-3.81 (m, 1H), | | | |
| | 3.50-3.23 (m, 4H), 2.58 (s, 2H), 2.40-2.26 (m, | | | |
| | 2H), 1.73-1.60 (m, 2H), 0.89 (s, 6H) | | | |
| 66 | 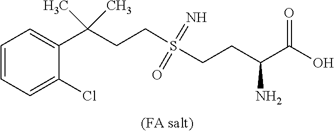 [SMILES: CC(C)(CCS(=N)(=O)CC[C@H](N)C(=O)O)c1ccccc1Cl] | 347.0 | 1.803 | C |
| | ¹H NMR (400 MHz, D₂O) δ 7.54-7.41 (m, 2H), | | | |
| | 7.38-7.20 (m, 2H), 4.15-4.04 (m, 1H), 4.03- | | | |
| | 3.76 (m, 2H), 3.49-3.28 (m, 2H), 2.70-2.49 (m, | | | |
| | 2H), 2.47-2.32 (m, 2H), 1.51 (s, 6H) | | | |

### Exemplary Embodiment 1e

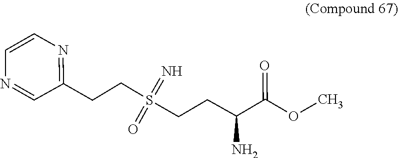 [SMILES: COC(=O)[C@@H](N)CCS(=N)(=O)CCc1cnccn1]

### Step 1

### (2S)-methyl 2-amino-4-(2-(pyrazin-2-yl)ethylsulfonimidoyl)butanoate

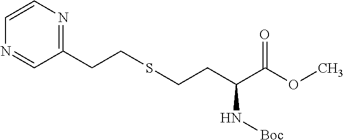 [SMILES: COC(=O)[C@H](CCSCCc1cnccn1)NC(=O)OC(C)(C)C]

A mixture of (S)-methyl 2-((tert-butoxycarbonyl)amino)-4-mercaptobutanoate (200 mg, 0.8 mmol) and 2-vinylpyrazine (170 mg, 1.6 mmol) in MeOH (2 mL) was stirred at 15° C. for 16 h under Ar. The mixture was concentrated and the residue was purified by prep-TLC (petroleum ether: ethyl acetate=1:1, R_{f}=0.12) to give (S)-methyl 2-((tert-butoxycarbonyl)amino)-4-((2-(pyrazin-2-yl)ethyl)thio)butanoate (0.25 g, 83% yield) as colorless oil. ¹H NMR (400 MHz, CDCl₃-d) δ 8.51 (br d, J=9.04 Hz, 2H), 8.45 (d, J=1.98 Hz, 1H), 5.13 (br d, J=7.06 Hz, 1H), 4.41 (br d, J=3.97 Hz, 1H), 3.75 (s, 3H), 3.16-3.05 (m, 2H), 3.02-2.92 (m, 2H), 2.65-2.52 (m, 2H), 2.20-2.07 (m, 1H), 1.96-1.85 (m, 1H), 1.45 (s, 9H).

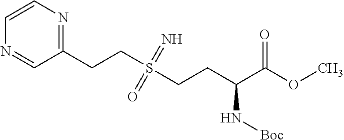 [SMILES: COC(=O)[C@H](CCS(=N)(=O)CCc1cnccn1)NC(=O)OC(C)(C)C]

To a mixture of (S)-methyl 2-((tert-butoxycarbonyl)amino)-4-((2-(pyrazin-2-yl)ethyl)thio)butanoate (200 mg, 563 umol) in MeOH (5 mL) was added PhI(OAc)₂ (453 mg, 1.41 mmol) ammonium carbamate (220 mg, 2.81 mmol) at 25° C. The mixture was stirred at 25° C. for 2 h, concentrated, and the residue purified by prep-TLC: (Ethyl acetate:Methanol=10:1, Rt=0.28) to give (2S)-methyl 2-((tert-butoxycarbonyl)amino)-4-(2-(pyrazin-2-yl)ethylsulfonimidoyl)butanoate (0.2 g, 92% yield) as colorless oil. ¹H NMR (400 MHz, CDCl₃-d) δ 8.58 (s, 1H), 8.54-8.51 (m, 1H), 8.49 (d, J=2.41 Hz, 1H), 5.34 (br s, 1H), 5.31 (s, 1H), 4.43 (br s, 1H), 3.78 (s, 3H), 3.68-3.59 (m, 2H), 3.50-3.36 (m, 2H), 3.29-3.14 (m, 2H), 2.53-2.41 (m, 1H), 2.29-2.15 (m, 1H), 1.45 (s, 9H).

To a mixture of (2S)-methyl 2-((tert-butoxycarbonyl)amino)-4-(2-(pyrazin-2-yl)ethylsulfonimidoyl)butanoate (50 mg, 129 mmol) in DCM (5 mL) was added TFA (1.54 g, 13.5 mmol, 1 mL) at 15° C. and the mixture was stirred at for 1 h. To the mixture was added toluene (50 mL) and concentrated to give a residue which was added H₂O (20 mL) and extracted with DCM (20 mL×2). The aqueous phase was lyophilized to give (2S)-methyl 2-amino-4-(2-(pyrazin-2-yl)ethylsulfonimidoyl)butanoate (26.44 mg, 44% yield, TFA) as light yellow oil. LCMS: Rt=0.916 min., (ES⁺) m/z (M+H)⁺=287.1; HPLC Conditions: A; ¹H NMR (400 MHz, D₂O) δ 8.63-8.59 (m, 2H), 8.54-8.49 (m, 1H), 2.65-2.44 (m, 2H), 4.40-4.29 (m, 1H), 4.20-4.07 (m, 2H), 4.00-3.86 (m, 2H), 3.84 (s, 2H), 3.81 (br s, 1H), 3.51 (br t, J=7.27 Hz, 2H).

### Exemplary Embodiment 1f

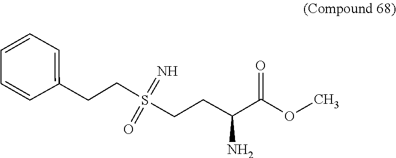 [SMILES: COC(=O)[C@@H](N)CCS(=N)(=O)CCc1ccccc1]

### (2S)-methyl 2-amino-4-(2-phenylethylsulfonimidoyl)butanoate

To a solution of (2S)-2-(tert-butoxycarbonylamino)-4-(2-phenylethylsulfonimidoyl)butanoic acid (140 mg, 378 mmol) in HCl/MeOH (4M, 3 mL) was stirred at 20° C. for 16 h. The mixture was concentrated, water (5 mL) was added, and extracted with DCM (5 mL×3). The aqueous phase was concentrated to give afford (2S)-methyl 2-amino-4-(2-phenylethylsulfonimidoyl)butanoate (110 mg, 84% yield, HCl) as a white solid. LCMS: Rt=2.049 min., (ES⁺) m/z (M+H)⁺=285.2; HPLC Conditions: A; ¹H NMR (400 MHz, D₂O) δ 7.46-7.29 (m, 5H), 4.26-4.16 (m, 1H), 4.02-3.91 (m, 2H), 3.83 (s, 3H), 3.71-3.56 (m, 2H), 3.25 (br t, J=7.8 Hz, 2H), 2.52-2.31 (m, 2H).

### Exemplary Embodiment 1g

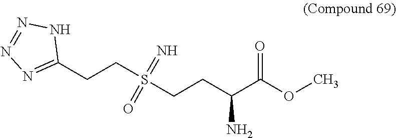 [SMILES: COC(=O)[C@@H](N)CCS(=N)(=O)CCc1nnn[nH]1]

### (2S)-methyl 4-(2-(1H-tetrazol-5-yl)ethylsulfonimidoyl)-2-aminobutanoate

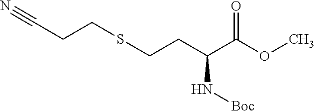 [SMILES: COC(=O)[C@H](CCSCCC#N)NC(=O)OC(C)(C)C]

To a solution of (S)-methyl 2-((tert-butoxycarbonyl)amino)-4-mercaptobutanoate (300 mg, 1.2 mmol) and 3-bromopropanenitrile (192 mg, 1.44 mmol, 118 mL) in DMF (3 mL) was added KI (398 mg, 2.4 mmol) and K₂CO₃ (497 mg, 3.6 mmol). The mixture was stirred at 20° C. for 16 h under Ar. The mixture was poured into water (20 mL) and extracted with EtOAc (10 mL×2). The combined organic extracts were dried over Na₂SO₄, filtered, and concentrated. The residue was purified by prep-TLC (SiO₂, Ethyl acetate:Methanol=1:1, R_{f}=0.62) to give (S)-methyl 2-((tert-butoxycarbonyl)amino)-4-((2-cyanoethyl)thio)butanoate (330 mg, 91% yield) as a yellow oil. ¹H NMR (400 MHz, CDCl₃-d) δ 5.12 (br s, 1H), 4.45 (br s, 1H), 3.88-3.69 (m, 3H), 2.90-2.75 (m, 2H), 2.66 (q, J=7.2 Hz, 4H), 2.13 (br s, 1H), 1.95 (td, J=7.2, 14.3 Hz, 1H), 1.51-1.41 (m, 9H).

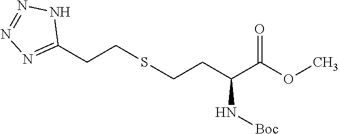 [SMILES: COC(=O)[C@H](CCSCCc1nnn[nH]1)NC(=O)OC(C)(C)C]

To a solution of (S)-methyl 2-((tert-butoxycarbonyl)amino)-4-((2-cyanoethyl)thio)butanoate (200 mg, 661 mmol) in dioxane (2 mL) was added dibutyl(oxo)tin (32.9 mg, 132 mmol) and TMSN₃ (228 mg, 1.98 mmol, 261 mL). The mixture was stirred at 120° C. for 5 h. The reaction was quenched by KF aqueous solution (2 mL) and the residue was purified by prep-HPLC (column: Phenomenex Gemini-NX C18 75×30 mm, 3 microns; mobile phase: [water (0.2% formic acid)-MeCN]; B %: 20%-50%, 12 min) to give (S)-methyl 4-((2-(1H-tetrazol-5-yl)ethyl)thio)-2-((tert-butoxycarbonyl)amino)butanoate (145 mg, 64% yield) as a yellow oil. ¹H NMR (400 MHz, CDCl₃-d) δ 14.34 (br s, 1H), 5.42 (br d, J=8.6 Hz, 1H), 4.68 (br d, J=4.2 Hz, 1H), 3.79 (s, 3H), 3.48-3.34 (m, 1H), 3.22-3.09 (m, 1H), 3.02-2.91 (m, 2H), 2.80 (br d, J=4.4 Hz, 1H), 2.73-2.61 (m, 1H), 2.13-1.88 (m, 2H), 1.48 (s, 9H).

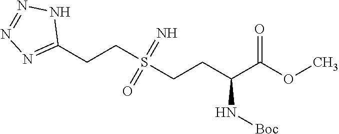 [SMILES: COC(=O)[C@H](CCS(=N)(=O)CCc1nnn[nH]1)NC(=O)OC(C)(C)C]

To a solution of (S)-methyl 4-((2-(1H-tetrazol-5-yl)ethyl)thio)-2-((tert-butoxycarbonyl)amino)butanoate (145 mg, 420 mmol) in MeOH (2 mL) was added PhI(OAc)₂ (270 mg, 840 mmol) and ammonium carbamate (163 mg, 2.10 mmol). The mixture was stirred at 20° C. for 3 h. The mixture was poured into water (10 mL) and extracted with EtOAc (15 mL×3). The aqueous phase was concentrated. The residue was purified by prep-HPLC (column: Phenomenex Luna C18 100×30 mm, 5 microns; mobile phase: [water (0.2% formic acid)-MeCN]; B %: 1%-30%, 10 min) to give (2S)-methyl 4-(2-(1H-tetrazol-5-yl)ethylsulfonimidoyl)-2-((tert-butoxycarbonyl)amino)butanoate (43 mg, 27% yield) as a white solid. ¹H NMR (400 MHz, D₂O) δ 4.33 (br s, 1H), 3.88-3.73 (m, 5H), 3.62-3.49 (m, 2H), 3.44-3.28 (m, 2H), 2.41 (br dd, J=5.5, 14.1 Hz, 1H), 2.29-2.10 (m, 1H), 1.44 (s, 9H).

To a solution of (2S)-methyl 4-(2-(1H-tetrazol-5-yl)ethylsulfonimidoyl)-2-((tert-butoxycarbonyl)amino)butanoate (10 mg, 26.5 mmol) in HCl/MeOH (1 mL) mixture was stirred at 20° C. for 1 h. The reaction mixture was concentrated to give (2S)-methyl 4-(2-(1H-tetrazol-5-yl)ethylsulfonimidoyl)-2-aminobutanoate (8.17 mg, 98% yield, HCl) as a yellow oil. LCMS: Rt=0.358 min., (ES⁺) m/z (M+H)⁺=277.0; HPLC Conditions: A; ¹H NMR (400 MHz, D₂O) δ 4.33 (br t, J=6.5 Hz, 1H), 3.96-3.78 (m, 5H), 3.64-3.44 (m, 4H), 2.55-2.40 (m, 2H).

### Exemplary Embodiment 1h

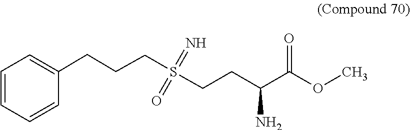 [SMILES: COC(=O)[C@@H](N)CCS(=N)(=O)CCCc1ccccc1]

### (2S)-methyl 2-amino-4-(3-phenylpropylsulfonimidoyl)butanoate

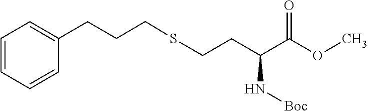 [SMILES: COC(=O)[C@H](CCSCCCc1ccccc1)NC(=O)OC(C)(C)C]

To a solution of (S)-methyl 2-((tert-butoxycarbonyl)amino)-4-mercaptobutanoate (200 mg, 0.8 mmol) in DMF (2 mL) was added K₂CO₃ (277 mg, 2.01 mmol) and 3-bromopropylbenzene (160 mg, 0.8 mmol, 121 mL) under argon. The mixture was stirred at 20° C. for 12 h. The reaction mixture was poured into water (6 mL), extracted with EtOAc (4 mL×2). The combined organic extracts were washed with brine (3 mL), dried over Na₂SO₄, filtered, and concentrated. The residue was purified by prep-TLC (SiO₂, Petroleum ether: ethyl acetate=3:1) to give (S)-methyl 2-((tert-butoxycarbonyl)amino)-4-((3-phenylpropyl)thio)butanoate (240 mg, 81% yield) as a yellow oil. ¹H NMR (400 MHz, CDCl₃-d) δ 7.32-7.27 (m, 2H), 7.23-7.21 (m, 3H), 5.10 (br d, J=6.4 Hz, 1H), 4.41 (br s, 1 H), 3.75 (s, 3H), 2.72 (t, J=7.6 Hz, 2H), 2.61-2.47 (m, 4H), 2.17-2.03 (m, 1H), 1.97-1.81 (m, 3H), 1.45 (s, 9H).

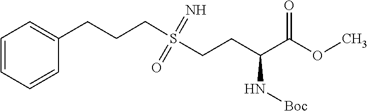 [SMILES: COC(=O)[C@H](CCS(=N)(=O)CCCc1ccccc1)NC(=O)OC(C)(C)C]

A mixture of (S)-methyl 2-((tert-butoxycarbonyl)amino)-4-((3-phenylpropyl)thio)butanoate (120 mg, 327 mmol), PhI(OAc)₂ (316 mg, 980 mmol) and ammonium carbamate (127 mg, 1.63 mmol) in MeOH (2 mL) was degassed and purged with N₂ for 3 times. The mixture was stirred at 30° C. for 16 h under N₂ atmosphere. The reaction mixture was concentrated and the residue was diluted with water (2 mL) and extracted with EtOAc (2 mL×2). The combined organic extracts were washed with brine (2 mL), dried over Na₂SO₄, filtered, and concentrated to give a residue. The residue was purified by prep-TLC (SiO₂, Ethyl acetate:Methanol=30:1) to give (2S)-methyl 2-((tert-butoxycarbonyl)amino)-4-(3-phenylpropylsulfonimidoyl)butanoate (40 mg, 31% yield) as a yellow oil. ¹H NMR (400 MHz, CDCl₃-d) δ 7.35-7.29 (m, 2H), 7.27-7.16 (m, 3H), 5.31 (br s, 1H), 4.41 (br s, 1H), 3.77 (s, 3H), 3.28-2.99 (m, 4H), 2.79 (br t, J=7.2 Hz, 2H), 2.38 (br d, J=10.0 Hz, 1H), 2.18 (br dd, J=7.2, 15.6 Hz, 3H), 1.45 (s, 9H).

A mixture of (2S)-methyl 2-((tert-butoxycarbonyl)amino)-4-(3-phenylpropylsulfonimidoyl)butanoate (40 mg, 100 mmol) in HCl/MeOH (1.5 mL, 4 M) was stirred at 17° C. for 1 h. The reaction mixture was concentrated to give a residue. The residue was diluted with water (1 mL) and extracted with DCM (0.5 mL×5). The aqueous phase was concentrated to give (2S)-methyl 2-amino-4-(3-phenylpropylsulfonimidoyl)butanoate (29 mg, 94% yield) as a yellow oil. LCMS: Rt=1.561 min, (ES⁺) m/z (M+H)⁺=299.1; HPLC Conditions: F; ¹H NMR (400 MHz, D₂O) δ 7.44-7.38 (m, 2H), 7.35-7.30 (m, 3H), 4.34 (dd, J=7.6, 5.6 Hz, 1H), 3.87 (s, 3H), 3.81-3.65 (m, 2H), 3.62-3.47 (m, 2H), 2.85 (t, J=7.2 Hz, 2H), 2.58-2.37 (m, 2H), 2.28-2.17 (m, 2H).

### Exemplary Embodiment 1i

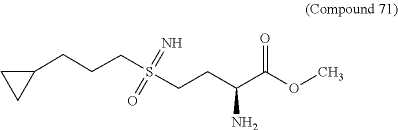 [SMILES: COC(=O)[C@@H](N)CCS(=N)(=O)CCCC1CC1]

### (2S)-methyl 2-amino-4-(3-cyclopropylpropylsulfonimidoyl)butanoate

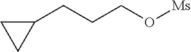 [SMILES: CS(=O)(=O)OCCCC1CC1]

To a mixture of 3-cyclopropylpropan-1-ol (100 mg, 998 mmol) in DCM (2 mL) was added MsCl (172 mg, 1.5 mmol, 116 mL) TEA (303 mg, 3 mmol, 417 mL) at 15° C. The mixture was stirred at 15° C. for 1 h and the mixture was quenched by sat. NaHCO₃ (20 mL) and extracted with DCM (20 mL×2). The combined organic phase was washed with brine (50 mL), dried with anhydrous Na₂SO₄, filtered, and concentrated. The mixture was purified by prep-TLC (petroleum ether: ethyl acetate=1:1, R_{f}=0.38) to give 3-cyclopropylpropyl methanesulfonate (80 mg, 45% yield) as colorless oil. ¹H NMR (400 MHz, CDCl₃-d) δ 4.28 (t, J=6.58 Hz, 2H), 3.05-2.97 (m, 3H), 1.87 (quin, J=7.02 Hz, 2H), 1.33 (q, J=7.23 Hz, 2H), 0.75-0.63 (m, 1H), 0.53-0.40 (m, 2H), 0.09-0.00 (m, 2H).

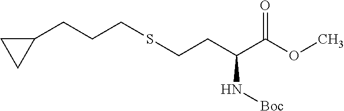 [SMILES: COC(=O)[C@H](CCSCCCC1CC1)NC(=O)OC(C)(C)C]

To a mixture of 3-cyclopropylpropyl methanesulfonate (70 mg, 393 mmol) (S)-methyl 2-((tert-butoxycarbonyl)amino)-4-mercaptobutanoate (97.9 mg, 393 mmol) in DMF (1 mL) was added K₂CO₃ (163 mg, 1.18 mmol) at 25° C. The mixture was stirred at 25° C. for 16 h under Ar. The mixture was added to H₂O (50 mL) and the aqueous phase was extracted with ethyl acetate (50 mL×2). The combined organic extracts were washed with brine (50 mL), dried with anhydrous Na₂SO₄, filtered, and concentrated. The residue was purified by silica gel chromatography (column height: 250 mm, diameter: 100 mm, 100-200 mesh silica gel, Petroleum ether/Ethyl acetate=10/1, 5/1) to give (S)-methyl 2-((tert-butoxycarbonyl)amino)-4-((3-cyclopropylpropyl)thio)butanoate (0.1 g, 77% yield) as colorless oil. ¹H NMR (400 MHz, CDCl₃-d) δ 5.11 (br s, 1H), 4.42 (br s, 1H), 3.76 (s, 3H), 2.56 (t, J=7.45 Hz, 4H), 2.13 (br d, J=8.33 Hz, 1H), 1.90 (qd, J=14.03, 7.23 Hz, 2H), 1.69 (quin, J=7.45 Hz, 2H), 1.46 (s, 9H), 1.38-1.25 (m, 3H), 0.73-0.61 (m, 1H), 0.47-0.41 (m, 2H), 0.07-0.01 (m, 2H).

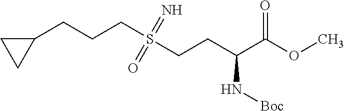 [SMILES: COC(=O)[C@H](CCS(=N)(=O)CCCC1CC1)NC(=O)OC(C)(C)C]

To a mixture of (S)-methyl 2-((tert-butoxycarbonyl)amino)-4-((3-cyclopropylpropyl)thio)butanoate (100 mg, 302 mmol) in MeOH (2 mL) was added PhI(OAc)₂ (243 mg, 754 mmol) ammonium carbamate (118 mg, 1.51 mmol) at 25° C. The mixture was stirred at 25° C. for 16 h, concentrated, and the residue was purified by prep-TLC (petroleum ether: ethyl acetate=0:1, R_{f}=0.13) to give (2S)-methyl 2-((tert-butoxycarbonyl)amino)-4-(3-cyclopropylpropylsulfonimidoyl)butanoate (0.04 g, 37% yield) as colorless oil. ¹H NMR (400 MHz, CDCl₃-d) δ 5.35 (br d, J=19.95 Hz, 1H), 4.45 (br s, 1H), 3.79 (s, 2H), 3.34-3.11 (m, 1H), 3.59-3.09 (m, 1H), 3.35-3.06 (m, 1H), 2.58-2.39 (m, 1H), 2.29-2.15 (m, 1H), 2.07-1.92 (m, 2H), 1.46 (s, 9H), 1.38 (q, J=7.31 Hz, 2H), 0.77-0.61 (m, 1H), 0.52-0.43 (m, 2H), 0.10-0.04 (m, 2H), 0.09-0.04 (m, 1H).

To a mixture of (2S)-methyl 2-((tert-butoxycarbonyl)amino)-4-(3-cyclopropylpropylsulfonimidoyl)butanoate (40 mg, 110 mmol) in DCM (1 mL) was added TFA (1.23 g, 10.8 mmol, 0.8 mL) at 15° C. and the mixture was stirred at 15° C. for 1 h. The mixture was concentrated and the residue was added H₂O (30 mL) and extracted with DCM (30 mL×2). The aqueous phase was lyophilized to give (2S)-methyl 2-amino-4-(3-cyclopropylpropylsulfonimidoyl)butanoate (31 mg, 73% yield, TFA) as colorless oil. LCMS: Rt=2.076 min., (ES⁺) m/z (M+H)⁺=263.1; HPLC Conditions: A; ¹H NMR (400 MHz, D₂O) δ 4.35 (dd, J=7.76, 5.69 Hz, 1H), 3.94-3.81 (m, 5H), 3.78-3.65 (m, 2H), 2.64-2.43 (m, 2H), 1.98 (quin, J=7.64 Hz, 2H), 1.46-1.33 (m, 2H), 0.74-0.64 (m, 1H), 0.48-0.39 (m, 2H), 0.07-0.02 (m, 2H).

### Exemplary Embodiment 1j

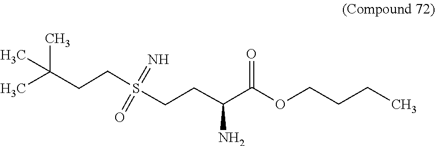 [SMILES: CCCCOC(=O)[C@@H](N)CCS(=N)(=O)CCC(C)(C)C]

### (2S)-butyl 2-amino-4-(3,3-dimethylbutylsulfonimidoyl)butanoate

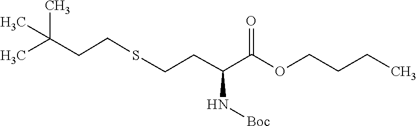 [SMILES: CCCCOC(=O)[C@H](CCSCCC(C)(C)C)NC(=O)OC(C)(C)C]

To a solution of butyl (tert-butoxycarbonyl)-L-homocysteinate (3.00 g, 10.3 mmol) in DMF was added K₂CO₃ (20.6 mmol), KI (10.3 mmol) and 1-bromo-3,3-dimethyl-butane (2.55 g, 15.4 mmol). The mixture was stirred at 20° C. for 16 h. The reaction mixture was filtered, and the filtrate was concentrated. The residue was purified by column chromatography (SiO₂, Petroleum ether: ethyl acetate=98:2 to 1:1) to give (S)-butyl 2-((tert-butoxycarbonyl)amino)-4-((3,3-dimethylbutyl)thio)butanoate (3.74 g, 97% yield) as a colorless oil. ¹H NMR (400 MHz, DMSO-d₆) δ 7.26 (d, J=7.9 Hz, 1H), 4.09-3.90 (m, 3H), 2.55-2.49 (m, 1H), 2.43-2.36 (m, 2H), 1.85-1.75 (m, 2H), 1.57-1.45 (m, 2H), 1.39-1.25 (m, 13H), 0.89-0.81 (m, 12H).

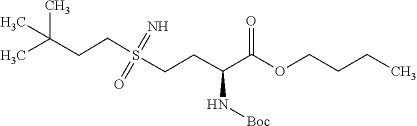 [SMILES: CCCCOC(=O)[C@H](CCS(=N)(=O)CCC(C)(C)C)NC(=O)OC(C)(C)C]

To a solution of (S)-butyl 2-((tert-butoxycarbonyl)amino)-4-((3,3-dimethylbutyl)thio)butanoate (1.8 g, 4.79 mmol) in MeOH was added PhI(OAc)₂ (4 eq) and ammonium carbamate (5 eq). The mixture was stirred at 20° C. for 6 h. The reaction mixture was concentrated, and the residue was diluted with water and extracted with DCM. The combined organic extracts were concentrated and purified by column chromatography to give (2S)-butyl 2-((tert-butoxycarbonyl)amino)-4-(3,3-dimethylbutylsulfonimidoyl)butanoate (1.57 g, 81% yield) as a colorless oil. ¹H NMR (400 MHz, MeOD-d₄) δ 4.27 (br dd, J=4.6, 8.2 Hz, 3H), 3.30 (br s, 1H), 3.29-3.06 (m, 4H), 2.39-2.27 (m, 1H), 2.21-2.08 (m, 1H), 1.78-1.63 (m, 5H), 1.50-1.39 (m, 11H), 1.04-0.94 (m, 12H).

To a solution of (2S)-butyl 2-((tert-butoxycarbonyl)amino)-4-(3,3-dimethylbutylsulfonimidoyl)butanoate (60 mg, 147 mmol) in HCl/dioxane (4N, 3 mL). The mixture was stirred at 20° C. for 12 h. The reaction mixture was concentrated and dried by lyophilization to afford (2S)-butyl 2-amino-4-(3,3-dimethylbutylsulfonimidoyl)butanoate (30 mg, 59% yield) as a white solid. LCMS: Rt=1.930 min., (ES⁺) m/z (M+H)⁺=307.1; HPLC Conditions: C; ¹H NMR (400 MHz, D₂O) δ 4.36-4.31 (m, 1H), 4.29 (t, J=6.5 Hz, 2H), 3.76-3.60 (m, 2H), 3.58-3.39 (m, 2H), 2.59-2.41 (m, 2H), 1.76-1.61 (m, 4H), 1.31 (s, 2H), 0.97-0.92 (m, 9H), 0.91-0.86 (m, 3H).

### Exemplary Embodiment 1k

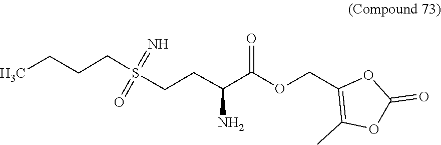 [SMILES: CCCCS(=N)(=O)CC[C@H](N)C(=O)OCc1oc(=O)oc1C]

### (2S)-(5-methyl-2-oxo-1,3-dioxol-4-yl)methyl 2-amino-4-(butylsulfonimidoyl) butanoate

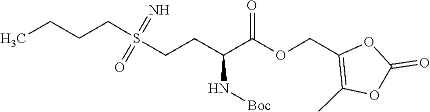 [SMILES: CCCCS(=N)(=O)CC[C@H](NC(=O)OC(C)(C)C)C(=O)OCc1oc(=O)oc1C]

To a solution of (2S)-2-((tert-butoxycarbonyl)amino)-4-(butylsulfonimidoyl)butanoic acid (0.1 g, 310 mmol) and 4-(bromomethyl)-5-methyl-1,3-dioxol-2-one (120 mg, 620 mmol) in DMF (2 mL) was added K₂CO₃ (85.7 mg, 620 mmol) and the mixture was stirred at 20° C. for 3 h. The mixture was poured into water (10 mL) and extracted with ethyl acetate (15 mL×2). The combined organic extracts were dried over Na₂SO₄, filtered, and concentrated. The residue was purified by prep-TLC (SiO₂, Petroleum ether: ethyl acetate=0:1, R_{f}=0.25) to give (2S)-(5-methyl-2-oxo-1,3-dioxol-4-yl)methyl 2-((tert-butoxycarbonyl)amino)-4-(butylsulfonimidoyl)butanoate (50 mg, 37% yield) as a yellow oil. ¹H NMR (400 MHz, CDCl₃-d) δ 5.50-5.28 (m, 1H), 4.94 (s, 2H), 4.44 (br dd, J=2.3, 6.7 Hz, 1H), 3.28-3.04 (m, 4H), 2.53-2.40 (m, 2H), 2.30-2.18 (m, 4H), 1.91-1.76 (m, 2H), 1.54-1.43 (m, 12H), 1.03-0.95 (in, 3H).

To a solution of (2S)-(5-methyl-2-oxo-1,3-dioxol-4-yl)methyl 2-((tert-butoxycarbonyl)amino)-4-(butylsulfonimidoyl)butanoate (50 mg, 115 mmol) in DCM (2 mL) and TFA (0.2 mL) was stirred at 20° C. for 2 h. The reaction mixture was concentrated at 35° C. Water (10 mL) was added and extracted with DCM (10 mL×3). The aqueous phase was lyophilized to give (2S)-(5-methyl-2-oxo-1,3-dioxol-4-yl)methyl 2-amino-4-(butylsulfonimidoyl)butanoate (37 mg, 67% yield, TFA) as a yellow solid. LCMS: Rt=2.075 min., (ES) m/z (M+H)⁺=335.0; HPLC Conditions: A; ¹H NMR (400 z, D₂O) δ 5.22-5.12 (i, 2H), 4.43-4.33 (, 1H), 3.77 (br t, J=7.9 Hz, 2H), 3.67-3.50 (i, 2H), 2.60-2.43 (m, 2H), 2.17 (s, 3H), 1.83 (quin, J 7.7 Hz, 2H), 1.48 (sxt, J 7.4 Hz, 2H), 0.92 (t, J=7.3 Hz, 3H).

The compounds described in Table 7 were prepared using the general methods outlined above.

TABLE 7
Characterization data for Compounds 74-99

| Compound | | MS | HPLC | HPLC |
|---|---|---|---|---|
| # | Structure | (M + H)+ | rt | cond. |
| 74 | 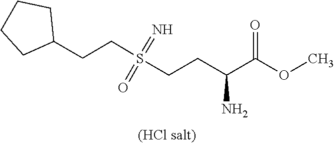 [SMILES: COC(=O)[C@@H](N)CCS(=N)(=O)CCC1CCCC1] | 277.1 | 2.475 | A |
| | ¹H NMR (400 MHz, D₂O) δ 4.35 (dd, 1H), | | | |
| | 3.91-3.77 (m, 5H), 3.71-3.57 (m, 2H), 2.62- | | | |
| | 2.41 (m, 2H), 1.97-1.71 (m, 5H), 1.67-1.44 | | | |
| | (m, 4H), 1.21-1.05 (m, 2H) | | | |
| 75 | 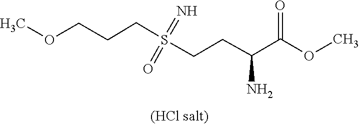 | 253.1 | 0.265 | A |
| | ¹H NMR (400 MHz, D₂O) δ = 4.39 (dd, 1H), | | | |
| | 3.90 (s, 3H), 3.86-3.74 (m, 2H), 3.71-3.56 | | | |
| | (m, 4H), 3.41-3.35 (m, 3H), 2.64-2.46 (m, | | | |
| | 2H), 2.17 (quin, 2H) | | | |
| 76 | 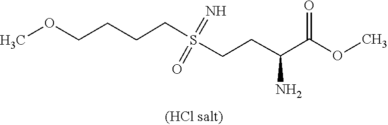 | 267.1 | 0.265 | A |
| | ¹H NMR (400 MHz, D₂O) δ 4.39 (dd, 1 H), | | | |
| | 3.90 (s, 3 H), 3.87-3.79 (m, 2 H), 3.66 (tq, 2 | | | |
| | H), 3.54 (t, 2 H), 3.36 (s, 3 H), 2.65-2.44 (m, | | | |
| | 2 H), 1.96 (quin, 2 H), 1.83-1.73 (m, 2 H) | | | |
| 77 | 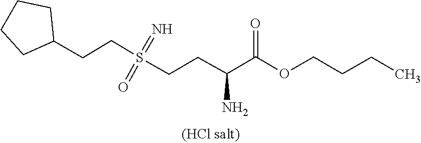 [SMILES: CCCCOC(=O)[C@@H](N)CCS(=N)(=O)CCC1CCCC1] | 319.1 | 1.958 | C |
| | ¹H NMR (400 MHz, D₂O) δ 4.36-4.17 (m, | | | |
| | 3H), 3.91-3.71 (m, 2H), 3.70-3.51 (m, 2H), | | | |
| | 2.58-2.37 (m, 2H), 1.94-1.69 (m, 5H), 1.67- | | | |
| | 1.39 (m, 6H), 1.31 (sxt, 2H), 1.16-0.99 (m, | | | |
| | 2H), 0.84 (t, 3H) | | | |
| 78 | 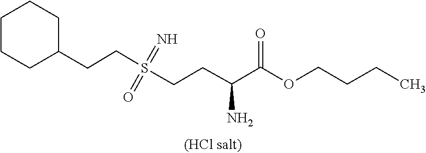 [SMILES: CCCCOC(=O)[C@@H](N)CCS(=N)(=O)CCC1CCCCC1] | 333.1 | 2.049 | C |
| | ¹H NMR (400 MHz, D₂O) δ 4.33-4.13 (m, | | | |
| | 3H), 3.72-3.57 (m, 2H), 3.56-3.41 (m, 2H), | | | |
| | 2.51-2.32 (m, 2H), 1.73-1.47 (m, 9H), 1.42- | | | |
| | 1.24 (m, 3H), 1.21-0.98 (m, 3H), 0.96-0.85 | | | |
| | (m, 2H), 0.83 (t, 3H) | | | |
| 79 | 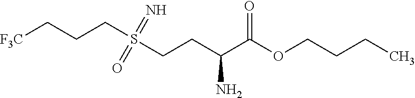 [SMILES: CCCCOC(=O)[C@@H](N)CCS(=N)(=O)CCCC(F)(F)F] | 333.0 | 1.826 | C |
| | ¹H NMR (400 MHz, D2O) δ 4.42-4.26 (m, | | | |
| | 3H), 4.00-3.71 (m, 4H), 2.64-2.38 (m, 4H), | | | |
| | 2.26-2.12 (m, 2H), 1.73-1.60 (m, 2H), 1.44- | | | |
| | 1.30 (m, 2H), 0.90 (t, 3H) | | | |
| 80 | 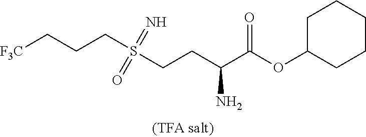 [SMILES: N=S(=O)(CCCC(F)(F)F)CC[C@H](N)C(=O)OC1CCCCC1] | 359.0 | 2.069 | F |
| | ¹H NMR (400 MHz, MeOD-d₄) δ 4.98-4.92 | | | |
| | (m, 1H), 4.30-4.19 (m, 1H), 3.49-3.34 (m, | | | |
| | 4H), 2.58-2.33 (m, 4H), 2.11 (quin, 2H), 1.94 | | | |
| | (br dd, 2H), 1.78 (br dd, 2H), 1.64-1.29 (m, 6H) | | | |
| 81 | 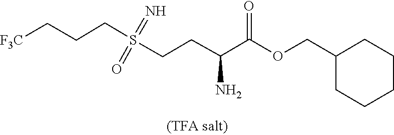 [SMILES: N=S(=O)(CCCC(F)(F)F)CC[C@H](N)C(=O)OCC1CCCCC1] | 373.1 | 2.157 | F |
| | ¹H NMR (400 MHz, MeOD-d₄) δ 4.38-4.26 | | | |
| | (m, 1H), 4.19-4.07 (m, 2H), 3.57-3.34 (m, | | | |
| | 4H), 2.60-2.35 (m, 4H), 2.20-2.06 (m, 2H), | | | |
| | 1.87-1.66 (m, 6H), 1.38-1.20 (m, 3H), 1.18- | | | |
| | 0.97 (m, 2H) | | | |
| 82 | 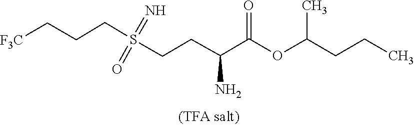 [SMILES: CCCC(C)OC(=O)[C@@H](N)CCS(=N)(=O)CCCC(F)(F)F] | 347.0 | 2.03 | F |
| | ¹H NMR (400 MHz, CDCl₃-d) δ 5.30-5.17 | | | |
| | (m, 1H), 5.07-4.92 (m, 1H), 4.33 (br d, 1H), | | | |
| | 3.64-3.28 (m, 4H), 2.53-2.39 (m, 1H), 2.38- | | | |
| | 2.25 (m, 2H), 2.24-2.09 (m, 3H), 1.69-1.62 | | | |
| | (m, 1H), 1.53-1.42 (m, 18H), 1.41-1.30 (m, | | | |
| | 2H), 1.28-1.24 (m, 3H), 0.94 (dt, 3H) | | | |
| 83 | 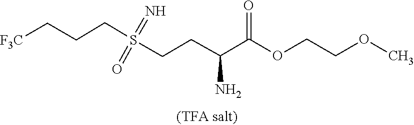 [SMILES: COCCOC(=O)[C@@H](N)CCS(=N)(=O)CCCC(F)(F)F] | 335.0 | 1.457 | C |
| | ¹H NMR (400 MHz, MeOD-d₄) δ 4.61-4.49 | | | |
| | (m, 1H), 4.43-4.28 (m, 2H), 3.74-3.65 (m, | | | |
| | 2H), 3.52-3.42 (m, 2H), 3.40 (s, 3H), 3.39- | | | |
| | 3.34 (m, 2H), 2.54-2.37 (m, 4H), 2.18-2.07 | | | |
| | (m, 2H) | | | |
| 84 | 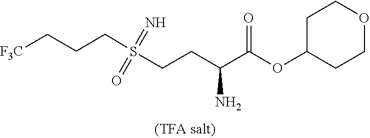 [SMILES: N=S(=O)(CCCC(F)(F)F)CC[C@H](N)C(=O)OC1CCOCC1] | 361.0 | 2.292 | A |
| | ¹H NMR (400 MHz, D₂O) δ 5.09 (tt, 1H), 4.38- | | | |
| | 4.24 (m, 1H), 4.11-3.80 (m, 6H), 3.63-3.50 | | | |
| | (m, 2H), 2.68-2.33 (m, 4H), 2.17 (quin, 2H), | | | |
| | 1.94 (br dd, 2H), 1.81-1.61 (m, 2H) | | | |
| 85 | 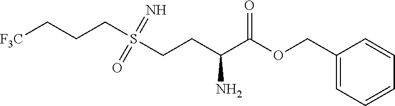 [SMILES: N=S(=O)(CCCC(F)(F)F)CC[C@H](N)C(=O)OCc1ccccc1] | 333.0 | 1.789 | C |
| | ¹H NMR (400 MHz, D₂O) δ 4.93-4.83 (m, | | | |
| | 1H), 3.67 (dt, 1H), 3.41-3.24 (m, 4H), 2.51- | | | |
| | 2.32 (m, 2H), 2.27-2.02 (m, 4H), 1.67-1.54 | | | |
| | (m, 2H), 1.23 (d, 3H), 0.87 (dt, 3H) | | | |
| 86 | 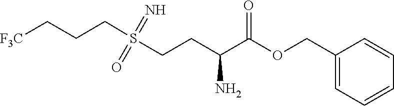 [SMILES: N=S(=O)(CCCC(F)(F)F)CC[C@H](N)C(=O)OCc1ccccc1] | 367.0 | 3.203 | A |
| | ¹H NMR (400 MHz, D2O) δ 7.48-7.38 (m, | | | |
| | 5H), 5.30-5.24 (m, 1H), 5.21-5.15 (m, 1H), | | | |
| | 3.74 (br s, 1H), 3.27-3.10 (m, 4H), 2.40-2.27 | | | |
| | (m, 2H), 2.21-2.05 (m, 2H), 1.98 (td, 2H) | | | |
| 87 | 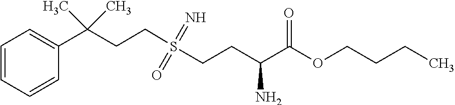 [SMILES: CCCCOC(=O)[C@@H](N)CCS(=N)(=O)CCC(C)(C)c1ccccc1] | 369.1 | 2.128 | F |
| | ¹H NMR (400 MHz, D₂O) δ 7.57-7.37 (m, | | | |
| | 4H), 7.36-7.25 (m, 1H), 4.37-4.18 (m, 3H), | | | |
| | 3.65-3.42 (m, 2H), 3.22-3.07 (m, 2H), 2.45- | | | |
| | 2.25 (m, 2H), 2.22-2.07 (m, 2H), 1.73-1.56 | | | |
| | (m, 2H), 1.38 (br s, 8H), 0.89 (br t, 3H) | | | |
| 88 | 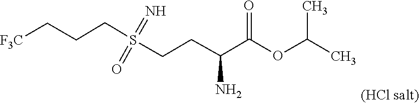 [SMILES: CC(C)OC(=O)[C@@H](N)CCS(=N)(=O)CCCC(F)(F)F] | 319.1 | 2.535 | A |
| | ¹H NMR (400 MHz, D₂O) δ 5.13 (quind, 1H), | | | |
| | 4.32-4.23 (m, 1H), 3.68-3.43 (m, 4H), 2.58- | | | |
| | 2.31 (m, 4H), 2.17-2.05 (m, 2H), 1.29 (dd, 6H) | | | |
| 89 | 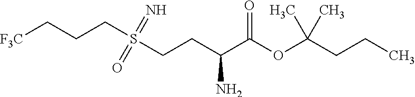 [SMILES: CCCC(C)(C)OC(=O)[C@@H](N)CCS(=N)(=O)CCCC(F)(F)F] | 361.2 | 2.735 | B |
| | ¹H NMR (400 MHz, D₂O) δ 3.57 (t, 1H), 3.41- | | | |
| | 3.19 (m, 4H), 2.46-2.30 (m, 2H), 2.23-2.12 | | | |
| | (m, 1H), 2.12-1.99 (m, 3H), 1.78-1.69 (m, | | | |
| | 2H), 1.43 (s, 6H), 1.37-1.24 (m, 2H), 0.87 (t, | | | |
| | 3H) | | | |
| 90 | 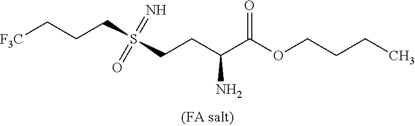 [SMILES: CCCCOC(=O)[C@@H](N)CC[S@@](=N)(=O)CCCC(F)(F)F] | 333.0 | 1.805 | C |
| | ¹H NMR (400 MHz, D₂O) δ 8.42 (s, 1H), 4.24 | | | |
| | (dt, 2H), 4.04 (br t, 1H), 3.51-3.31 (m, 4H), | | | |
| | 2.46-2.22 (m, 4H), 2.08 (quin, 2H), 1.75- | | | |
| | 1.60 (m, 2H), 1.37 (qd, 2H), 0.89 (t, 3H) | | | |
| 91 | 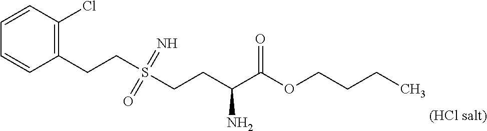 [SMILES: CCCCOC(=O)[C@@H](N)CCS(=N)(=O)CCc1ccccc1Cl] | 361.0 | 1.994 | C |
| | ¹H NMR (400 MHz, D₂O) δ 7.51-7.45 (m, | | | |
| | 1H), 7.44-7.39 (m, 1H), 7.35-7.30 (m, 2H), | | | |
| | 4.27 (t, 3H), 3.95-3.84 (m, 2H), 3.76-3.64 | | | |
| | (m, 2H), 3.40-3.28 (m, 2H), 2.58-2.39 (m, | | | |
| | 2H), 1.73-1.58 (m, 2H), 1.34 (qd, 2H), 0.87 (t, | | | |
| | 3H) | | | |
| 92 | 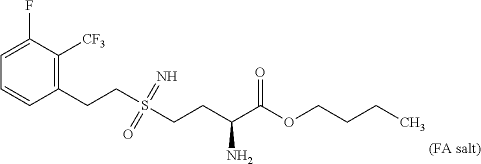 [SMILES: CCCCOC(=O)[C@@H](N)CCS(=N)(=O)CCc1cccc(F)c1C(F)(F)F] | 413.2 | 2.007 | C |
| | ¹H NMR (400 MHz, D₂O) δ 7.63-7.52 (m, | | | |
| | 1H), 7.32-7.16 (m, 2H), 4.27-4.16 (m, 2H), | | | |
| | 4.01 (q, 1H), 3.57-3.48 (m, 2H), 3.43-3.28 | | | |
| | (m, 4H), 2.43-2.19 (m, 2H), 1.69-1.54 (m, | | | |
| | 2H), 1.33 (qd, 2H), 0.85 (t, 3H) | | | |
| 93 | 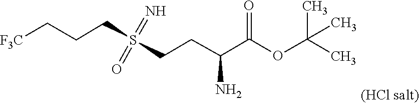 [SMILES: CC(C)(C)OC(=O)[C@@H](N)CC[S@@](=N)(=O)CCCC(F)(F)F] | 333.1 | 2.849 | A |
| | ¹H NMR (400 MHz, D₂O) δ 5.28-5.20 (m, | | | |
| | 1H), 4.36-4.21 (m, 1H), 3.26-3.00 (m, 4H), | | | |
| | 2.45-2.26 (m, 3H), 2.22-2.09 (m, 3H), 1.47 | | | |
| | (d, 18H) | | | |
| 94 | 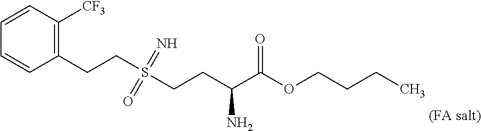 [SMILES: CCCCOC(=O)[C@@H](N)CCS(=N)(=O)CCc1ccccc1C(F)(F)F] | 395.1 | 2.033 | C |
| | ¹H NMR (400 MHz, D₂O) δ 7.71 (d, 1H), 7.61- | | | |
| | 7.53 (m, 1H), 7.51-7.39 (m, 2H), 4.33-4.19 | | | |
| | (m, 3H), 3.74-3.55 (m, 4H), 3.34-3.22 (m, | | | |
| | 2H), 2.55-2.37 (m, 2H), 1.68-1.52 (m, 2H), | | | |
| | 1.38-1.23 (m, 2H), 0.81 (t, 3H). | | | |
| 95 | 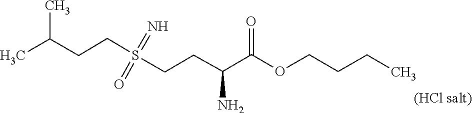 [SMILES: CCCCOC(=O)[C@@H](N)CCS(=N)(=O)CCC(C)C] | 293.1 | 1.806 | C |
| | ¹H NMR (400 MHz, D₂O) δ 4.37-4.16 (m, | | | |
| | 3H), 3.92-3.72 (m, 2H), 3.70-3.50 (m, 2H), | | | |
| | 2.59-2.36 (m, 2H), 1.80-1.54 (m, 5H), 1.31 | | | |
| | (qd, 2H), 1.07-0.76 (m, 9H) | | | |
| 96 | 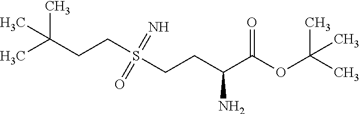 [SMILES: CC(C)(C)CCS(=N)(=O)CC[C@H](N)C(=O)OC(C)(C)C] | 307.1 | 1.888 | C |
| | ¹H NMR (400 MHz, MeOD) δ 3.55-3.45 (m, | | | |
| | 1H), 3.30-3.18 (m, 2H), 3.17-3.04 (m, 2H), | | | |
| | 2.31-2.13 (m, 1H), 2.10-1.94 (m, 1H), 1.82- | | | |
| | 1.64 (m, 2H), 1.52 (s, 9H), 1.00 (s, 9H). | | | |
| 97 | 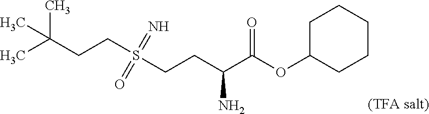 [SMILES: CC(C)(C)CCS(=N)(=O)CC[C@H](N)C(=O)OC1CCCCC1] | 333.1 | 3.534 | A |
| | ¹H NMR (400 MHz, D₂O) δ 4.95-4.86 (m, | | | |
| | 1H), 4.28 (ddd, 1H), 3.86-3.66 (m, 2H), 3.65- | | | |
| | 3.48 (m, 2H), 2.57-2.39 (m, 2H), 1.82 (br s, | | | |
| | 2H), 1.76-1.58 (m, 4H), 1.57-1.42 (m, 3H), | | | |
| | 1.42-1.22 (m, 3H), 0.91 (s, 9H). | | | |
| 98 | 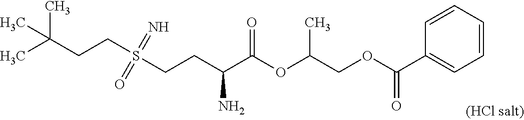 [SMILES: CC(COC(=O)c1ccccc1)OC(=O)[C@@H](N)CCS(=N)(=O)CCC(C)(C)C] | 413.1 | 2.043 | C |
| | ¹H NMR (400 MHz, MeOD) δ 8.07 (d, 2H), | | | |
| | 7.72-7.61 (m, 1H), 7.59-7.49 (m, 2H), 5.61- | | | |
| | 5.42 (m, 1H), 4.75-4.59 (m, 1H), 4.57-4.43 | | | |
| | (m, 1H), 4.40-4.31 (m, 1H), 4.25-4.02 (m, | | | |
| | 2H), 3.98-3.71 (m, 2H), 2.72-2.43 (m, 2H), | | | |
| | 1.91-1.77 (m, 2H), 1.54-1.41 (m, 3H), 1.10- | | | |
| | 0.98 (m, 9H) | | | |
| 99 | 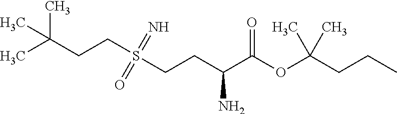 [SMILES: CCCC(C)(C)OC(=O)[C@@H](N)CCS(=N)(=O)CCC(C)(C)C] | 335.1 | 2.129 | C |
| | ¹H NMR (400 MHz, MeOD) δ 3.50 (ddd, 1H), | | | |
| | 3.30-3.19 (m, 2H), 3.17-3.08 (m, 2H), 2.30- | | | |
| | 2.17 (m, 1H), 2.08-1.96 (m, 1H), 1.84-1.67 | | | |
| | (m, 4H), 1.49 (s, 6H), 1.45-1.34 (m, 2H), 1.03- | | | |
| | 0.91 (m, 12H) | | | |

### Exemplary Embodiment l1

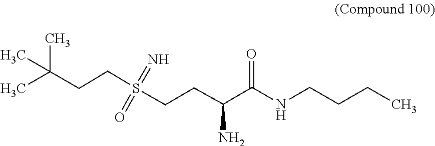 [SMILES: CCCCNC(=O)[C@@H](N)CCS(=N)(=O)CCC(C)(C)C]

### (2S)-2-amino-N-butyl-4-(3,3-dimethylbutylsulfonimidoyl)butanamide

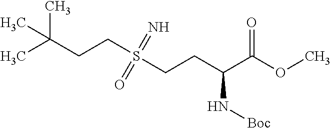 [SMILES: COC(=O)[C@H](CCS(=N)(=O)CCC(C)(C)C)NC(=O)OC(C)(C)C]

To a solution of methyl N-(tert-butoxycarbonyl)-S-(3,3-dimethylbutyl)-L-homocysteinate (3 g, 9 mmol) in MeOH (24 mL) was added PhI(OAc)₂ (11.9 g, 36.0 mmol) and ammonium carbamate (5.62 g, 71.9 mmol). The mixture was stirred at 20° C. for 16 h then concentrated. The residue was added water (30 mL) and extracted with ethyl acetate (45 mL×3). The combined organic extracts were dried over Na₂SO₄, filtered, and concentrated. The residue was purified by column chromatography (SiO₂, Petroleum ether: ethyl acetate=1:1) to give (2S)-methyl 2-((tert-butoxycarbonyl)amino)-4-(3,3-dimethylbutylsulfonimidoyl)butanoate (1.6 g, 49% yield) as a colorless oil. ¹H NMR (400 MHz, CDCl₃-d) δ 5.39-5.23 (m, 1H), 4.44 (br s, 1H), 3.79 (s, 3H), 3.23-2.94 (m, 4H), 2.50-2.36 (m, 1H), 2.24-2.10 (m, 1H), 1.78-1.67 (m, 2H), 1.46 (s, 9H), 0.96 (s, 9H).

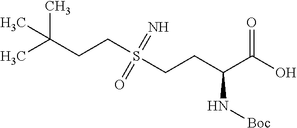 [SMILES: CC(C)(C)CCS(=N)(=O)CC[C@H](NC(=O)OC(C)(C)C)C(=O)O]

To a solution of (2S)-methyl 2-((tert-butoxycarbonyl)amino)-4-(3,3-dimethylbutylsulfonimidoyl)butanoate (1.60 g, 4.39 mmol) in THE (12 mL) and H₂O (4 mL) was added LiOH·H₂O (368 mg, 8.78 mmol). The mixture was stirred at 25° C. for 2 h. The solution was adjusted to pH˜5 with citric acid and extracted with ethyl acetate (20 mL×4). The combined organic extracts were dried over Na₂SO₄, filtered, and concentrated to give (2S)-methyl 2-((tert-butoxycarbonyl)amino)-4-(3,3-dimethylbutylsulfonimidoyl)butanoate (1.30 g, 85% yield) as a white solid. ¹H NMR (400 MHz, CDCl₃-d) S 5.69 (br d, J=6.5 Hz, 1H), 4.55-4.37 (m, 1H), 3.53-3.17 (m, 4H), 2.47-2.21 (m, 2H), 1.82-1.65 (m, 2H), 1.44 (s, 9H), 0.96 (d, J=3.4 Hz, 9H).

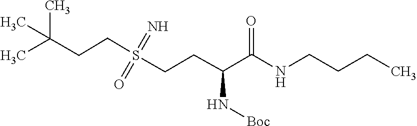 [SMILES: CCCCNC(=O)[C@H](CCS(=N)(=O)CCC(C)(C)C)NC(=O)OC(C)(C)C]

To a mixture of (2S)-methyl 2-((tert-butoxycarbonyl)amino)-4-(3,3-dimethylbutylsulfonimidoyl)butanoate (300 mg, 855 mmol), butan-1-amine (313 mg, 4.28 mmol) and DIEA (331 mg, 2.57 mmol) in DMF (5 mL) was added HATU (488 mg, 1.28 mmol). The mixture was stirred at 20° C. for 2 h then poured into water (10 ml) and extracted with ethyl acetate (5 mL×3). The combined organic extracts were washed with brine (10 ml), dried over Na₂SO₄, filtered, and concentrated to give a residue. The residue was purified by prep-HPLC (column: Phenomenex Phenomenex Luna C18 100×30 mm×5 um; mobile phase: [water(0.2% FA)-MeCN]; B %: 30%-50%, 12 min) to give tert-butyl ((2S)-1-(butylamino)-4-(3,3-dimethylbutylsulfonimidoyl)-1-oxobutan-2-yl)carbamate (80.0 mg, 23% yield) as a white solid. ¹H NMR (400 MHz, MeOD-d₄) δ 4.26-4.18 (m, 1H), 3.70-3.47 (m, 4H), 3.23 (br t, J=6.9 Hz, 2H), 2.33 (br s, 1H), 2.22-2.08 (m, 1H), 1.84-1.74 (m, 2H), 1.57-1.46 (m, 11H), 1.43-1.29 (m, 3H), 1.03 (s, 9H), 0.96 (t, J=7.3 Hz, 3H)

A solution of tert-butyl ((2S)-1-(butylamino)-4-(3,3-dimethylbutylsulfonimidoyl)-1-oxobutan-2-yl)carbamate (40 mg, 98.6 mmol) in HCl/dioxane (4 M, 4.0 mL) was stirred at 20° C. for 1 h. The reaction mixture was concentrated. The residue was dissolved in water (1 mL), extracted with DCM (1 mL×2). The aqueous phase was lyophilized to give (2S)-2-amino-N-butyl-4-(3,3-dimethylbutylsulfonimidoyl)butanamide (25 mg, 74% yield) as a white solid. LCMS: Rt=1.852 min, (ES⁺) m/z (M+H)⁺=306.1; HPLC Conditions: C; ¹H NMR (400 MHz, D₂O) δ 4.10 (br d, J=1.6 Hz, 1H), 3.61-3.49 (m, 4H), 3.30-3.15 (m, 2H), 2.45-2.37 (m, 2H), 1.72-1.65 (m, 2H), 1.47 (quin, J=7.2 Hz, 2H), 1.28 (d, J=7.6 Hz, 2H), 0.91 (s, 9H), 0.88-0.82 (m, 3H).

### Exemplary Embodiment 1m

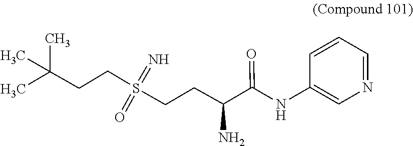 [SMILES: CC(C)(C)CCS(=N)(=O)CC[C@H](N)C(=O)Nc1cccnc1]

### (2S)-2-amino-4-(3,3-dimethylbutylsulfonimidoyl)-N-(pyridin-3-yl)butanamide

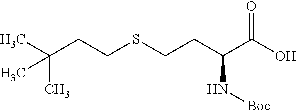 [SMILES: CC(C)(C)CCSCC[C@H](NC(=O)OC(C)(C)C)C(=O)O]

To a solution of methyl N-(tert-butoxycarbonyl)-S-(3,3-dimethylbutyl)-L-homocysteinate (4.7 g, 14.1 mmol) in THE (35 mL) and H₂O (7 mL) was added LiOH·H₂O (1.18 g, 28.9 mmol). The mixture was stirred at 25° C. for 2 h. The solution was adjusted to pH=5 with citric acid and extracted with ethyl acetate. The combined organic extracts were dried over Na₂SO₄, filtered, and concentrated to give (S)-2-((tert-butoxycarbonyl)amino)-4-((3,3-dimethylbutyl)thio)butanoic acid (4.40 g, 98% yield), as a yellow oil. ¹H NMR (400 MHz, CDCl₃-d) δ=5.28-5.12 (m, 1H), 4.47-4.30 (m, 1H), 2.74-2.37 (m, 4H), 2.23-2.08 (m, 1H), 2.03-1.85 (m, 1H), 1.50-1.40 (m, 11H), 0.94-0.85 (m, 9H).

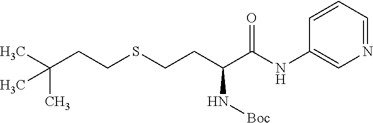 [SMILES: CC(C)(C)CCSCC[C@H](NC(=O)OC(C)(C)C)C(=O)Nc1cccnc1]

A mixture of (S)-2-((tert-butoxycarbonyl)amino)-4-((3,3-dimethylbutyl)thio)butanoic acid (300 mg, 939 mmol), pyridin-3-amine (442 mg, 4.70 mmol), HATU (714 mg, 1.88 mmol) and DIEA (364 mg, 2.82 mmol, 491 mL) in DMF (5 mL) was stirred at 50° C. for 3 h. The mixture was added to H₂O (50 mL) and the aqueous phase was extracted with ethyl acetate (50 mL×2). The combined organic phases were washed with brine (100 mL), dried with anhydrous Na₂SO₄, filtered, and concentrated. The residue was purified by silica gel chromatography (petroleum ether/ethyl acetate=10/1 to 1/1) to give (S)-tert-butyl (4-((3,3-dimethylbutyl)thio)-1-oxo-1-(pyridin-3-ylamino)butan-2-yl)carbamate (0.45 g, crude), as a light yellow oil. ¹H NMR (400 MHz, CDCl₃-d) δ 8.61 (s, 2H), 8.37 (br d, J=4.52 Hz, 1H), 8.12 (br d, J=7.95 Hz, 1H), 5.21 (br d, J=6.97 Hz, 1H), 4.44 (br d, J=7.70 Hz, 1H), 2.76-2.59 (m, 2H), 2.57-2.47 (m, 2H), 2.29-2.15 (m, 1H), 2.04-1.96 (m, 1H), 1.48 (d, J=1.71 Hz, 9H), 1.34-1.22 (m, 2H), 0.91 (d, J=1.71 Hz, 9H).

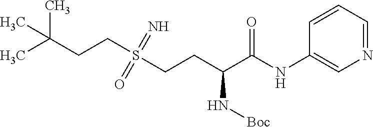 [SMILES: CC(C)(C)CCS(=N)(=O)CC[C@H](NC(=O)OC(C)(C)C)C(=O)Nc1cccnc1]

To a solution of S)-tert-butyl (4-((3,3-dimethylbutyl)thio)-1-oxo-1-(pyridin-3-ylamino)butan-2-yl)carbamate (200 mg, 506 mmol) in i-PrOH (2 mL) was added PhI(OAc)₂ (407 mg, 1.26 mmol) and ammonium carbamate (197 mg, 2.53 mmol) and the mixture was stirred at 25° C. for 2 h. The mixture was added to H₂O (50 mL) and extracted with ethyl acetate (50 mL×2). The combined organic phases were washed with brine (50 mL), dried with anhydrous Na₂SO₄, filtered, and concentratedc. The residue was purified by silica gel chromatography (petroleum ether/ethyl acetate=0/1) to give tert-butyl ((2S)-4-(3,3-dimethylbutylsulfonimidoyl)-1-oxo-1-(pyridin-3-ylamino)butan-2-yl)carbamate (0.14 g, 65% yield) as colorless oil. ¹H NMR (400 MHz, CDCl₃-d) δ 10.28-9.87 (m, 1H), 8.75-8.67 (m, 1H), 8.37 (d, J=4.77 Hz, 1H), 8.13 (br dd, J=19.13, 8.29 Hz, 1H), 6.07-5.67 (m, 1H), 4.91-4.75 (m, 1H), 3.32 (br d, J=2.86 Hz, 2H), 3.18 (br dd, J=12.76, 8.70 Hz, 1H), 2.53-2.28 (m, 2H), 2.10 (br s, 1H), 1.83-1.67 (m, 2H), 1.47 (d, J=4.53 Hz, 9H), 0.98 (d, J=7.99 Hz, 9H).

To a solution of tert-butyl ((2S)-4-(3,3-dimethylbutylsulfonimidoyl)-1-oxo-1-(pyridin-3-ylamino)butan-2-yl)carbamate (50 mg, 117 mmol) in DCM (3 mL) was added TFA (462 mg, 4.05 mmol, 300 mL) at 25° C. and the mixture was stirred at 25° C. for 2 h. The mixture was concentrated, the residue was added H₂O (20 mL) and extracted with DCM (10 mL×2). The aqueous phase was lyophilized to give (2S)-2-amino-4-(3,3-dimethylbutylsulfonimidoyl)-N-(pyridin-3-yl)butanamide (51 mg, 99% yield, TFA) as light yellow oil. LCMS: Rt=2.208 min, (ES⁺) m/z (M+H)⁺=327.0; HPLC Conditions: E; ¹H NMR (400 MHz, D₂O) δ 9.29 (br s, 1H), 9.36-9.23 (m, 1H), 8.63-8.47 (m, 2H), 8.12-7.98 (m, 1H), 4.47 (br s, 1H), 3.83-3.66 (m, 2H), 3.64-3.48 (m, 2H), 2.72-2.51 (m, 2H), 1.70 (br t, J=8.25 Hz, 2H), 0.90 (s, 9H).

### Exemplary Embodiment 1n (Peak 1) & 1n (Peak 2)

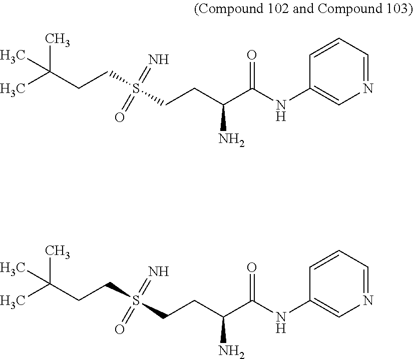 [SMILES: CC(C)(C)CC[S@@](=N)(=O)CC[C@H](N)C(=O)Nc1cccnc1 ; CC(C)(C)CC[S@](=N)(=O)CC[C@H](N)C(=O)Nc1cccnc1]

### (S)-2-amino-4-((S)-3,3-dimethylbutylsulfonimidoyl)-N-(pyridin-3-yl)butanamide (Compound 102) & (S)-2-amino-4-((R)-3,3-dimethylbutylsulfonimidoyl)-N-(pyridin-3-yl)butanamide (Compound 103)

(2S)-2-amino-4-(3,3-dimethylbutylsulfonimidoyl)-N-(pyridin-3-yl)butanamide (30 mg) was purified by SFC: (column: DAICEL CHIRALPAK IF (250 mm×30 mm, 10 um); mobile phase: [0.1% NH3H2O MeOH]; B %: 60%-60%, 10 min) to give (S)-2-amino-4-((S)-3,3-dimethylbutylsulfonimidoyl)-N-(pyridin-3-yl)butanamide & (S)-2-amino-4-((R)-3,3-dimethylbutylsulfonimidoyl)-N-(pyridin-3-yl)butanamide. The separated compounds were tentatively assigned stereochemistry and notated by their SFC peak elution order.

### (S)-2-amino-4-((R)-3,3-dimethylbutylsulfonimidoyl)-N-(pyridin-3-yl)butanamide (Peak 1) (Compound 102)

(4.27 mg) as colorless oil. LCMS: Rt=0.278 min, (ES⁺) m/z (M+H)⁺=327.1; HPLC Conditions: A; ¹H NMR (400 MHz, MeOD-d₄) δ 8.79 (d, J=2.38 Hz, 1H), 8.28 (dd, J=4.89, 1.31 Hz, 1H), 8.19-8.13 (m, 1H), 7.42 (dd, J=8.17, 4.83 Hz, 1H), 3.63 (dd, J=7.39, 5.60 Hz, 1H), 3.30-3.19 (m, 2H), 3.12 (ddd, J=11.92, 5.25, 3.58 Hz, 2H), 2.29 (ddt, J=13.72, 10.80, 5.50, 5.50 Hz, 1H), 2.18-2.00 (m, 1H), 1.74-1.68 (m, 2H), 0.96 (s, 9H),

### (S)-2-amino-4-((S)-3,3-dimethylbutylsulfonimidoyl)-N-(pyridin-3-yl)butanamide (Peak 2) (Compound 103)

(5.3 mg) as colorless oil. LCMS: Rt=0.279 min, (ES⁺) m/z (M+H)⁺=327.1; HPLC Conditions: A; ¹H NMR (400 MHz, MeOD-d₄) δ 8.79 (br s, 1H), 8.28 (br d, J=4.17 Hz, 1H), 8.16 (br d, J=8.11 Hz, 1H), 7.42 (dd, J=8.23, 4.77 Hz, 1H), 3.66 (br t, J=6.14 Hz, 1H), 3.30-3.25 (m, 2H), 3.19-3.06 (m, 2H), 2.34-2.23 (m, 1H), 2.17-2.05 (m, 1H), 1.78-1.65 (m, 2H), 0.99-0.94 (m, 9H).

### Exemplary Embodiment 1o

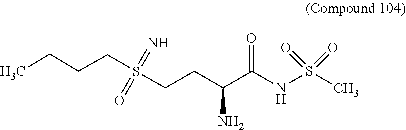 [SMILES: CCCCS(=N)(=O)CC[C@H](N)C(=O)NS(C)(=O)=O]

### (2S)-2-amino-4-(butylsulfonimidoyl)-N-(methylsulfonyl)butanamide

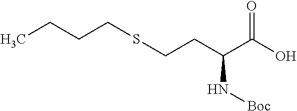 [SMILES: CCCCSCC[C@H](NC(=O)OC(C)(C)C)C(=O)O]

To a solution of methyl (2S)-2-(tert-butoxycarbonylamino)-4-butylsulfanyl-butanoate (300 mg, 982 mmol) in THE (2.5 mL) and H₂O (0.5 mL) was added LiOH·H₂O (82.4 mg, 1.96 mmol). The mixture was stirred at 60° C. for 1 h. The solution was adjusted to pH˜6 with HCl and extracted with DCM (30 mL×2). The combined organic extracts were dried over Na₂SO₄, filtered, and concentrated to afford (S)-2-((tert-butoxycarbonyl)amino)-4-(butylthio)butanoic acid (280 mg, 98% yield) as a yellow oil. ¹H NMR (400 MHz, CDCl₃-d) δ 5.13 (br s, 1H), 4.44 (br s, 1H), 2.70-2.46 (m, 4H), 2.18 (br s, 1H), 1.98 (qd, J=7.4, 14.4 Hz, 1H), 1.62-1.53 (m, 2H), 1.48-1.42 (m, 11H), 0.93 (t, J=7.3 Hz, 3H).

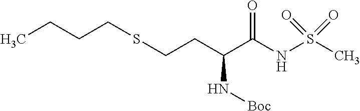 [SMILES: CCCCSCC[C@H](NC(=O)OC(C)(C)C)C(=O)NS(C)(=O)=O]

To a solution of (S)-2-((tert-butoxycarbonyl)amino)-4-(butylthio)butanoic acid (250 mg, 858 mmol) and MeSO₂NH₂ (101 mg, 1.03 mmol) in DCM (5 mL) was added EDCI (197 mg, 1.03 mmol) and DMAP (126 mg, 1.03 mmol). The mixture was stirred at 15° C. for 16 h. The solution was adjusted to pH˜7 with HCl (0.5 M) and extracted with DCM (20 mL×2). The combined organic extracts were dried over Na₂SO₄, filtered, and concentrated. The residue was purified by prep-TLC (SiO₂, Dichloromethane:Methanol=10:1) to give (S)-tert-butyl (4-(butylthio)-1-(methylsulfonamido)-1-oxobutan-2-yl)carbamate (210 mg, 66% yield) as a colorless oil. ¹H NMR (400 MHz, CDCl₃-d) δ 5.18 (br d, J=7.1 Hz, 1H), 4.31 (br d, J=6.5 Hz, 1H), 3.30 (s, 3H), 2.72-2.44 (m, 4H), 2.18 (qd, J=6.9, 13.8 Hz, 1H), 2.02-1.90 (m, 1H), 1.66-1.52 (m, 3H), 1.50-1.27 (m, 11H), 1.03-0.88 (m, 3H).

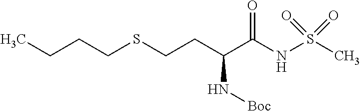 [SMILES: CCCCSCC[C@H](NC(=O)OC(C)(C)C)C(=O)NS(C)(=O)=O]

To a solution of (S)-tert-butyl (4-(butylthio)-1-(methylsulfonamido)-1-oxobutan-2-yl)carbamate (210 mg, 570 mmol) in DCM (5 mL) was added m-CPBA (116 mg, 570 mmol, 85% purity). The mixture was stirred at 15° C. for 1 h. The reaction was quenched with sat. aq. NaHCO₃ (10 mL) and then extracted with DCM (10 mL×2). The combined organic phase was washed with Na₂SO₃ (5 mL), dried over anhydrous Na₂SO₄, filtered, and concentrated. The residue was purified by prep-TLC (Ethyl acetate:Methanol=10:1, R_{f}=0.2). The aqueous phase was also concentrated and the residue was purified by prep-HPLC (column: Phenomenex Gemini-NX C18 75×30 mm, 3 microns; mobile phase: [water (10 mM NH4HCO3)-MeCN]; B %: 5%-30%, 12 min) to give tert-butyl ((2S)-4-(butylsulfinyl)-1-(methylsulfonamido)-1-oxobutan-2-yl)carbamate (150 mg, 68% yield) as a yellow oil. ¹H NMR (400 MHz, CDCl₃-d) δ 6.11-5.69 (m, 1H), 4.35-4.09 (m, 1H), 3.14-2.98 (m, 3H), 2.83 (br s, 3H), 2.75-2.63 (m, 1H), 2.34 (br s, 1H), 2.18 (s, 1H), 1.77-1.62 (m, 2H), 1.52-1.42 (m, 11H), 0.96 (t, J=7.3 Hz, 3H).

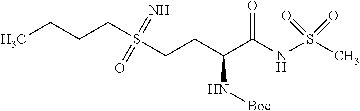 [SMILES: CCCCS(=N)(=O)CC[C@H](NC(=O)OC(C)(C)C)C(=O)NS(C)(=O)=O]

To a solution of tert-butyl ((2S)-4-(butylsulfinyl)-1-(methylsulfonamido)-1-oxobutan-2-yl)carbamate (50.0 mg, 130 mmol) in MeOH (2 mL) was added PhI(OAc)₂ (126 mg, 390 mmol) and ammonium carbamate (50.7 mg, 650 mmol). The mixture was stirred at 15° C. for 16 h. The mixture was poured into water (5 mL) and extracted with EA (10 mL×3). The combined organic phase was washed with Na₂SO₃ (5 mL), dried over anhydrous Na₂SO₄, filtered, and concentrated to give tert-butyl ((2S)-4-(butylsulfonimidoyl)-1-(methylsulfonamido)-1-oxobutan-2-yl)carbamate (50 mg, 96% yield) as a yellow oil. ¹H NMR (400 MHz, CDCl₃-d) δ 5.94 (br s, 1H), 4.20 (br s, 1H), 3.32-3.07 (m, 4H), 3.03 (s, 3H), 2.35 (br s, 1H), 2.17 (br s, 1H), 1.81-1.69 (m, 2H), 1.51-1.39 (m, 11H), 1.01-0.87 (m, 3H). To a solution of tert-butyl ((2S)-4-(butylsulfonimidoyl)-1-(methylsulfonamido)-1-oxobutan-2-yl)carbamate (40 mg, 100 mmol) in DCM (1 mL) and TFA (0.1 mL). The mixture was stirred at 15° C. for 8 h. The mixture was concentrated and the residue was purified by prep-HPLC (column: Phenomenex Gemini-NX C18 75×30 mm, 3 microns; mobile phase: [water (0.2% FA)-MeCN]; B %: 1%-15%, 12 min) to give (2S)-2-amino-4-(butylsulfonimidoyl)-N-(methylsulfonyl)butanamide. (9 mg, 26% yield, FA) as a white solid. LCMS: Rt=0.267 min., (ES⁺) m/z (M+H)⁺=300.10; HPLC Conditions: A; ¹H NMR (400 MHz, D₂O) δ 3.97-3.84 (m, 1H), 3.54-3.18 (m, 4H), 3.05 (s, 3H), 2.36 (q, J=7.4 Hz, 2H), 1.75 (quin, J=7.7 Hz, 2H), 1.44 (sxt, J=7.4 Hz, 2H), 0.91 (t, J=7.4 Hz, 3H).

### Exemplary Embodiment 1p

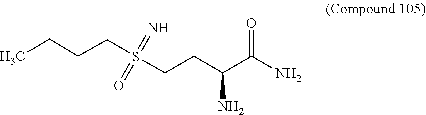

### (2S)-2-amino-4-(butylsulfonimidoyl)butanamide

To a solution of (2S)-methyl 2-((tert-butoxycarbonyl)amino)-4-(butylsulfonimidoyl)butanoate (100 mg, 297 mmol) in NH₃/MEOH (7 M, 3 mL) was stirred at 30° C. for 16 h in a sealed tube. The reaction mixture was concentrated to give ((2S)-1-amino-4-(butylsulfonimidoyl)-1-oxobutan-2-yl)carbamate (90 mg, 94% yield), as a white solid. ¹H NMR (400 MHz, CDCl₃-d) δ 7.16-6.73 (m, 1H), 5.92-5.61 (m, 1H), 5.52 (br s, 1H), 4.60-4.42 (m, 1H), 3.33-3.03 (m, 4H), 2.38-2.24 (m, 2H), 1.91-1.78 (m, 2H), 1.55-1.44 (m, 11H), 0.99 (t, J=7.3 Hz, 3H).

To a solution of ((2S)-1-amino-4-(butylsulfonimidoyl)-1-oxobutan-2-yl)carbamate (20 mg, 62.2 mmol) in HCl/dioxane (4 M, 1 mL) was stirred at 20° C. for 2 h. The reaction mixture was concentrated and the residue was triturated with CH₃CN (2 mL), filtered, and the filter cake was concentrated to give (2S)-2-amino-4-(butylsulfonimidoyl)butanamide (10 mg, 56% yield, HCl) as a white solid. LCMS: Rt=0.451 min. (ES⁺) m/z (M+H)⁺=222.1; HPLC Conditions: A; ¹H NMR (400 MHz, D₂O) δ 4.17 (br s, 1H), 3.71-3.45 (m, 4H), 2.42 (br d, J=6.0 Hz, 2H), 1.77 (br d, J=7.3 Hz, 2H), 1.42 (br d, J=6.7 Hz, 2H), 0.87 (br s, 3H).

The compounds described in Table 8 were prepared using the general methods

TABLE 8
Characterization of Compounds 106-133

| Compound | | MS | HPLC | HPLC |
|---|---|---|---|---|
| # | Structure | (M + H)+ | rt | cond. |
| 106 | 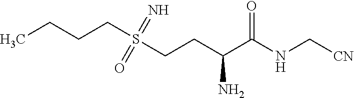 [SMILES: CCCCS(=N)(=O)CC[C@H](N)C(=O)NCC#N] | 261.1 | 1.598 | I |
| | ¹H NMR (400 MHz, D₂O) δ 4.21 (s, 2H), 3.59 | | | |
| | (t, 1H), 3.32-−3.14 (m, 4H), 2.22-−1.97 (m, | | | |
| | 2H), 1.73 (quin, 2H), 1.43 (sxt, 2H), 0.90 (t, | | | |
| | 3H) | | | |
| 107 | 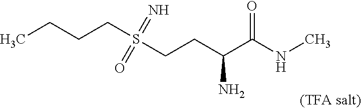 | 236.1 | 0.269 | A |
| | ¹H NMR (400 MHz, D₂O) δ 4.13 (t, 1H), | | | |
| | 3.75-−3.54 (m, 4 H), 2.78 (s, 3 H), 2.59-−2.38 | | | |
| | (m, 2 H), 1.82 (quin, 2 H), 1.53-−1.38 (m, 2 | | | |
| | H), 0.92 (t, 3 H). | | | |
| 108 | 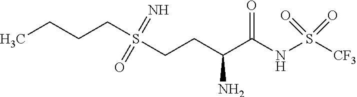 [SMILES: CCCCS(=N)(=O)CC[C@H](N)C(=O)NS(=O)(=O)C(F)(F)F] | 354.1 | 1.826 | B |
| | ¹H NMR (400 MHz, MeOD-d₄) δ 3.96-−3.83 | | | |
| | (m, 1H), 3.50-−3.35 (m, 1H), 3.32-−3.22 (m, | | | |
| | 1H), 3.21-−3.07 (m, 2H), 2.47-−2.25 (m, 2H), | | | |
| | 1.88-−1.72 (m, 2H), 1.51 (sxt, 2H), 1.01 (t, | | | |
| | 3H). | | | |
| 109 | 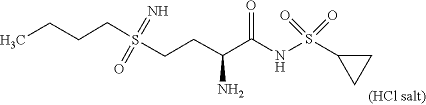 [SMILES: CCCCS(=N)(=O)CC[C@H](N)C(=O)NS(=O)(=O)C1CC1] | 326.0 | 0.605 | C |
| | ¹H NMR (400 MHz, D₂O) δ = 4.06-−3.99 (m, | | | |
| | 1H), 3.96-−3.76 (m, 2H), 3.75-−3.64 (m, 2H), | | | |
| | 2.88-−2.74 (m, 1H), 2.54-−2.34 (m, 2H), | | | |
| | 1.88-−1.77 (m, 2H), 1.45 (sxt, 2H), 1.12-−0.97 | | | |
| | (m, 4H), 0.88 (t, 3H). | | | |
| 110 | 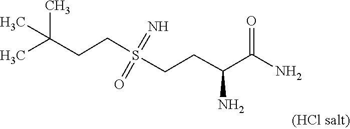 [SMILES: CC(C)(C)CCS(=N)(=O)CC[C@H](N)C(N)=O] | 250.1 | 1.856 | A |
| | ¹H NMR (400 MHz, D₂O) δ 4.20 (t, 1H), | | | |
| | 3.74-−3.61 (m, 2H), 3.60-−3.48 (m, 2H), | | | |
| | 2.51-−2.40 (m, 2H), 1.70 (br t, 2H), 0.91 (s, | | | |
| | 9H) | | | |
| 111 | 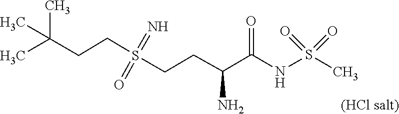 [SMILES: CC(C)(C)CCS(=N)(=O)CC[C@H](N)C(=O)NS(C)(=O)=O] | 328.0 | 1.269 | C |
| | ¹H NMR (400 MHz, D₂O) δ 4.03-−3.94 (m, | | | |
| | 1H), 3.93-−3.61 (m, 4H), 3.13-−3.02 (m, 3H), | | | |
| | 2.49-−2.36 (m, 2H), 1.73 (t, 2H), 0.95-−0.84 | | | |
| | (m, 9H) | | | |
| 112 | 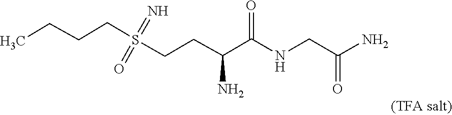 [SMILES: CCCCS(=N)(=O)CC[C@H](N)C(=O)NCC(N)=O] | 279.1 | 0.268 | A |
| | ¹H NMR (400 MHz, D₂O) δ 4.25 (t, 1H), 4.07- | | | |
| | −3.93 (m, 2H), 3.92-−3.80 (m, 2H), 3.80-−3.61 | | | |
| | (m, 2H), 2.58-−2.41 (m, 2H), 1.84 (quin, 2H), | | | |
| | 1.47 (sxt, 2H), 0.90 (t, 3H) | | | |
| 113 | 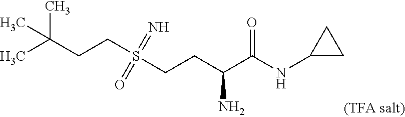 [SMILES: CC(C)(C)CCS(=N)(=O)CC[C@H](N)C(=O)NC1CC1] | 290.1 | 2.466 | A |
| | ¹HNMR (400 MHz, D₂O) δ 4.06 (t, 1H), 3.75- | | | |
| | −3.55 (m, 4H), 2.65 (tt, 1H), 2.49-−2.36 (m, | | | |
| | 2H), 1.71 (t, 2H), 0.92 (s, 9H), 0.77 (q, 2H), | | | |
| | 0.57-−0.48 (m, 2H). | | | |
| 114 | 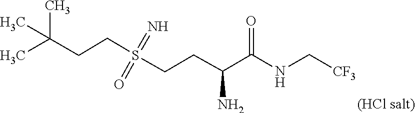 [SMILES: CC(C)(C)CCS(=N)(=O)CC[C@H](N)C(=O)NCC(F)(F)F] | 332.1 | 1.784 | C |
| | ¹H NMR (400 MHz, D₂O) δ 4.21 (t, 1H), 4.10- | | | |
| | −3.79 (m, 2H), 3.74-−3.47 (m, 4H), 2.55-−2.32 | | | |
| | (m, 2H), 1.67 (t, 2H), 0.88 (s, 9H). | | | |
| 115 | 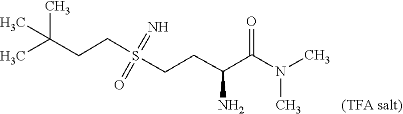 [SMILES: CN(C)C(=O)[C@@H](N)CCS(=N)(=O)CCC(C)(C)C] | 278.1 | 2.281 | A |
| | ¹H NMR (400 MHz, D₂O) δ 4.72 (s, 1H), 3.84- | | | |
| | −3.57 (m, 4H), 3.13 (s, 3H), 2.99 (s, 3H), 2.55- | | | |
| | −2.40 (m, 2H), 1.80-−1.69 (m, 2H), 0.96- | | | |
| | −0.94 (m, 1H), 0.95 (s, 9H) | | | |
| 116 | 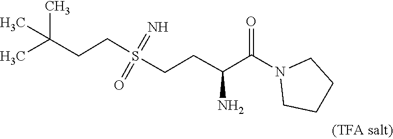 [SMILES: CC(C)(C)CCS(=N)(=O)CC[C@H](N)C(=O)N1CCCC1] | 304.1 | 1.661 | C |
| | ¹H NMR (400 MHz, D₂O) δ 4.49 (t, 1H), 3.88- | | | |
| | −3.52 (m, 5H), 3.51-−3.32 (m, 3H), 2.46 (td, | | | |
| | 2H), 2.01-−1.78 (m, 4H), 1.77-−1.65 (m, 2H), | | | |
| | 0.91 (s, 9H). | | | |
| 117 | 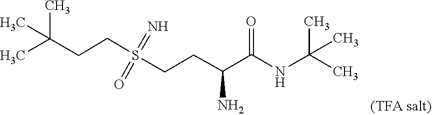 [SMILES: CC(C)(C)CCS(=N)(=O)CC[C@H](N)C(=O)NC(C)(C)C] | 306.1 | 2.928 | A |
| | ¹H NMR (400 MHz, D₂O) δ = 4.01 (t, 1H), | | | |
| | 3.82-−3.62 (m, 4H), 2.52-−2.36 (m, 2H), | | | |
| | 1.78-−1.67 (m, 2H), 1.29 (s, 9H), 0.91 (s, 9H) | | | |
| 118 | 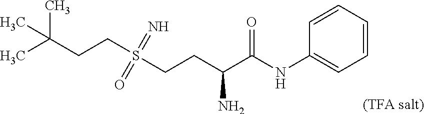 [SMILES: CC(C)(C)CCS(=N)(=O)CC[C@H](N)C(=O)Nc1ccccc1] | 326.1 | 1.801 | C |
| | ¹H NMR (400 MHz, D₂O) δ 7.50-−7.40 (m, | | | |
| | 4H), 7.32-−7.24 (m, 1H), 4.35 (br s, 1H), 3.68 | | | |
| | (br d, 2H), 3.52 (br d, 2H), 2.57 (br d, 2H), | | | |
| | 1.67 (br t, 2H), 0.88 (br s, 9H) | | | |
| 119 | 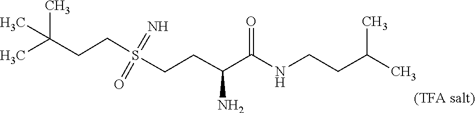 [SMILES: CC(C)CCNC(=O)[C@@H](N)CCS(=N)(=O)CCC(C)(C)C] | 320.1 | 3.472 | E |
| | ¹H NMR (400 MHz, D₂O) δ 0.89-−0.82 (m, 6 | | | |
| | H), 0.96-−0.90 (m, 9 H), 1.44-−1.34 (m, 2 H), | | | |
| | 1.60-−1.47 (m, 1 H), 1.78-−1.63 (m, 2 H), | | | |
| | 2.52-−2.35 (m, 2 H), 3.37-−3.14 (m, 2 H), | | | |
| | 3.72-−3.50 (m, 4 H), 4.160-−4.05 (m, 1 H). | | | |
| 120 | 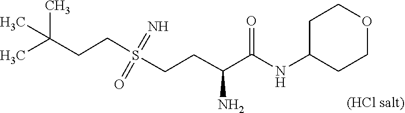 [SMILES: CC(C)(C)CCS(=N)(=O)CC[C@H](N)C(=O)NC1CCOCC1] | 334.0 | 1.666 | C |
| | ¹H NMR (400 MHz, MeOD) δ 4.19-−4.11 (m, | | | |
| | 1H), 4.09-−3.80 (m, 7H), 3.51 (dt, 2H), 2.63- | | | |
| | −2.41 (m, 2H), 1.98-−1.81 (m, 4H), 1.62 (dt, | | | |
| | 2H), 1.06 (s, 9H). | | | |
| 121 | 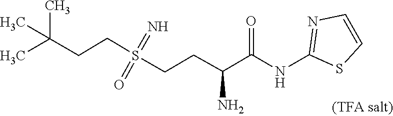 [SMILES: CC(C)(C)CCS(=N)(=O)CC[C@H](N)C(=O)Nc1nccs1] | 330.0 | 1.607 | C |
| | ¹H NMR (400 MHz, D₂O) δ 7.44 (d, 1H), 7.18 | | | |
| | (d, 1H), 4.41-−4.33 (m, 1H), 3.62-−3.51 (m, | | | |
| | 2H) 3.44-−3.35 (m, 2H), 2.56-−2.46 (m, 2H), | | | |
| | 1.65-−1.58 (m, 2H), 0.85 (s, 9H) | | | |
| 122 | 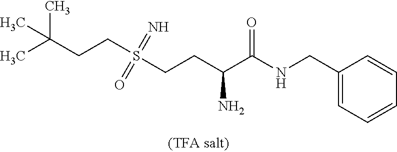 [SMILES: CC(C)(C)CCS(=N)(=O)CC[C@H](N)C(=O)NCc1ccccc1] | 340.1 | 3.293 | A |
| | ¹H NMR (400 MHz, D₂O) δ 0.98-−0.82 (m, 9 | | | |
| | H), 1.73-−1.56 (m, 2 H), 2.56-−2.36 (m, 2 H), | | | |
| | 3.66-−3.40 (m, 4 H), 4.24-−4.12 (m, 1 H), | | | |
| | 4.57-−4.28 (m, 2 H), 7.51-−7.26 (m, 5 H). | | | |
| 123 | 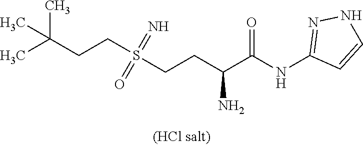 [SMILES: CC(C)(C)CCS(=N)(=O)CC[C@H](N)C(=O)Nc1cc[nH]n1] | 316.1 | 2.314 | A |
| | ¹H NMR (400 MHz, D₂O) δ ppm 7.52-−7.91 | | | |
| | (m, 1 H), 6.41-−6.53 (m, 1 H), 4.21-−4.44 (m, | | | |
| | 1 H), 3.37-−3.92 (m, 4 H), 2.38-−2.79 (m, 2 | | | |
| | H), 1.57-−1.81 (m, 2 H), 0.79-−1.00 (m, 9 H) | | | |
| 124 | 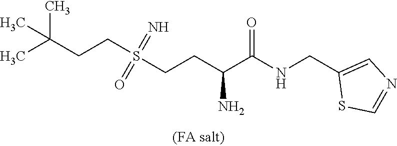 [SMILES: CC(C)(C)CCS(=N)(=O)CC[C@H](N)C(=O)NCc1cncs1] | 347.0 | 1.495 | C |
| | ¹H NMR (400 MHz, D₂O) δ 9.47 (d, 1H), | | | |
| | 8.09-−8.02 (m, 1H), 4.73 (br s, 2H), 4.23 (t, | | | |
| | 1H), 3.27-−3.56 (m, 4H), 2.55-−2.44 (m, 2H), | | | |
| | 1.72-−1.64 (m, 2H), 0.92 (s, 9H) | | | |
| 125 | 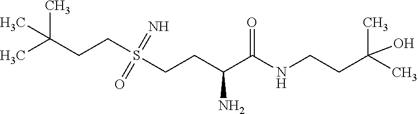 [SMILES: CC(C)(C)CCS(=N)(=O)CC[C@H](N)C(=O)NCCC(C)(C)O] | 336.1 | 1.659 | A |
| | ¹H NMR (400 MHz, MeOD) δ 8.49 (br s, 1H), | | | |
| | 4.11-−3.80 (m, 1H), 3.38 (br d, 2H), 3.31- | | | |
| | −3.07 (m, 4H), 2.31 (br s, 2H), 1.73 (br s, 4H), | | | |
| | 1.25 (s, 6H), 1.00 (s, 9H) | | | |
| 126 | 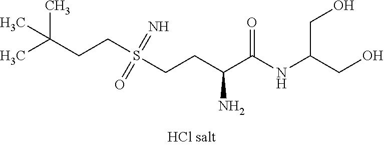 [SMILES: CC(C)(C)CCS(=N)(=O)CC[C@H](N)C(=O)NC(CO)CO] | 324.1 | 2.096 | A |
| | ¹H NMR (400 MHz, D₂O) δ 4.07-−3.95 (m, | | | |
| | 1H), 3.72-−3.65 (m, 2H), 3.64-−3.52 (m, 3H), | | | |
| | 3.33-−3.17 (m, 4H), 2.18-−1.98 (m, 2H), | | | |
| | 1.70-−1.57 (m, 2H), 0.92 (s, 9H). | | | |
| 127 | 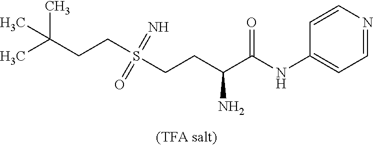 [SMILES: CC(C)(C)CCS(=N)(=O)CC[C@H](N)C(=O)Nc1ccncc1] | 327.2 | 2.044 | B |
| | ¹H NMR (400 MHz, D₂O) δ = 8.63 (d, 2H), | | | |
| | 8.15 (dd, 2H), 4.52 (ddd, 1H), 3.72-−3.62 (m, | | | |
| | 2H), 3.58-−3.43 (m, 2H), 2.72-−2.51 (m, 2H), | | | |
| | 1.78-−1.58 (m, 2H), 0.91 (d, 9H) | | | |
| 128 | 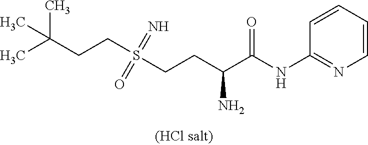 [SMILES: CC(C)(C)CCS(=N)(=O)CC[C@H](N)C(=O)Nc1ccccn1] | 327.1 | 2.71 | A |
| | ¹H NMR (400 MHz, D₂O) δ 8.27-−8.45 (m, 2 | | | |
| | H), 7.51-−7.68 (m, 2 H), 4.46-−4.57 (m, 1 H), | | | |
| | 4.43-−4.57 (m, 1 H), 3.49-−3.91 (m, 4 H), | | | |
| | 2.48-−2.76 (m, 2 H), 1.60-−1.76 (m, 2 H), | | | |
| | 0.82-−0.90 (m 9 H) | | | |
| 129 | 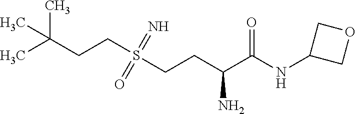 [SMILES: CC(C)(C)CCS(=N)(=O)CC[C@H](N)C(=O)NC1COC1] | 306.1 | 2.322 | A |
| | ¹H NMR (400 MHz, D₂O) δ 4.98-−4.87 (m, | | | |
| | 3H), 4.67-−4.59 (m, 2H), 3.53 (t, 1H), 3.33- | | | |
| | −3.16 (m, 4H), 2.19-−1.96 (m, 2H), 1.70-−1.54 | | | |
| | (m, 2H), 0.92 (s, 9H) | | | |
| 130 | 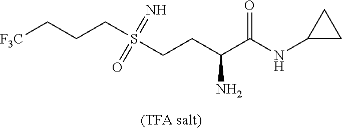 [SMILES: N=S(=O)(CCCC(F)(F)F)CC[C@H](N)C(=O)NC1CC1] | 316.0 | 2.074 | A |
| | ¹H NMR (400 MHz, D₂O) δ 4.07 (td, 1H), | | | |
| | 3.90-−3.65 (m, 4H), 2.65 (tt, 1H), 2.52-−2.33 | | | |
| | (m, 4H), 2.16 (quin, 2H), 0.83-−0.69 (m, 2H), | | | |
| | 0.58-−0.48 (m, 2H) | | | |
| 131 | 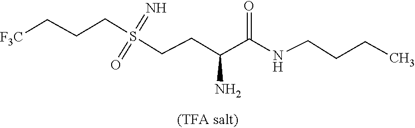 [SMILES: CCCCNC(=O)[C@@H](N)CCS(=N)(=O)CCCC(F)(F)F] | 332.1 | 2.769 | A |
| | ¹H NMR (400 MHz, D₂O) δ 4.13 (t, 1H), 3.75- | | | |
| | −3.50 (m, 4H), 3.29-−3.12 (m, 2H), 2.53- | | | |
| | −2.27 (m, 4H), 2.22-−2.02 (m, 2H), 1.61-−1.42 | | | |
| | (m, 2H), 1.38-−1.21 (m, 2H), 0.86 (t, 3H). | | | |
| 132 | 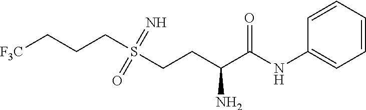 [SMILES: N=S(=O)(CCCC(F)(F)F)CC[C@H](N)C(=O)Nc1ccccc1] | 352.0 | 2.944 | A |
| | ¹H NMR (400 MHz, D₂O) δ 7.58-−7.38 (m, 4 | | | |
| | H), 7.37-−7.21 (m, 1 H), 4.44-−4.29 (m, 1 H), | | | |
| | 3.83-−3.57 (m, 4 H), 2.71-−2.50 (m, 2 H), | | | |
| | 2.48-−2.29 (m, 2 H), 2.22-−2.07 (m, 2 H) | | | |
| 133 | 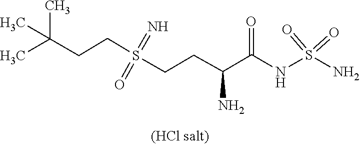 [SMILES: CC(C)(C)CCS(=N)(=O)CC[C@H](N)C(=O)NS(N)(=O)=O] | 355.0 | 0.772 | A |
| | ¹H NMR (400 MHz, D₂O) δ 4.20-−4.13 (m, | | | |
| | 1H), 3.80-−3.49 (m, 4H), 2.54-−2.32 (m, 4H), | | | |
| | 2.12 (quin, 2H) | | | |

### Exemplary Embodiment 1q

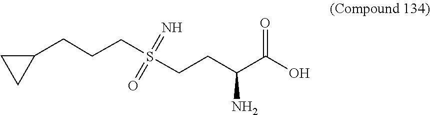 [SMILES: N=S(=O)(CCCC1CC1)CC[C@H](N)C(=O)O]

### (2S)-2-amino-4-(3-cyclopropylpropylsulfonimidoyl)butanoic acid

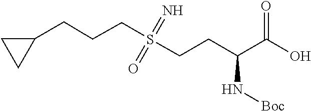 [SMILES: CC(C)(C)OC(=O)N[C@@H](CCS(=N)(=O)CCCC1CC1)C(=O)O]

To a mixture of compound (2S)-methyl 2-((tert-butoxycarbonyl)amino)-4-(3-cyclopropylpropylsulfonimidoyl)butanoate (See Ex 71) (100 mg, 276 mmol) in THE (2 mL) was added LiOH·H₂O (34.7 mg, 828 mmol) in H₂O (0.4 mL) at 20° C., the mixture was stirred at 20° C. for 2 h. The mixture was concentrated to remove THF, to the aqueous phase was added HCl (1M) to adjust the pH=5 then extracted with DCM (20 mL×3). The combined organic phase was washed with brine (50 mL), dried with anhydrous Na₂SO₄, filtered, and concentrated to give compound (2S)-2-((tert-butoxycarbonyl)amino)-4-(3-cyclopropylpropylsulfonimidoyl)butanoic acid (0.06 g, 64% yield) as colorless oil. ¹H NMR (400 MHz, CDCl₃-d) δ 5.86-5.58 (m, 1H), 4.65-4.44 (m, 1H), 3.43 (br s, 4H), 3.32 (br d, J=8.11 Hz, 1H), 2.42 (br s, 2H), 2.00 (br d, J=7.23 Hz, 2H), 1.47-1.36 (m, 11H), 0.70 (br d, J=6.14 Hz, 1H), 0.52-0.43 (m, 2H), 0.08 (br d, J=3.51 Hz, 2H).

A mixture of (2S)-2-((tert-butoxycarbonyl)amino)-4-(3-cyclopropylpropylsulfonimidoyl)butanoic acid (60.0 mg, 172 umol) in TFA (0.3 mL) DCM (3 mL) was stirred at 20° C. for 1 h. The mixture was concentrated, and the residue was added H₂O (20 mL) and extracted with DCM (20.0 mL×2). The aqueous phase was lyophilized to give (2S)-2-amino-4-(3-cyclopropylpropylsulfonimidoyl)butanoic acid (Compound 134) (50.8 mg, 81% yield, TFA) as colorless oil. LCMS: Rt=1.500 min, (ES⁺) m/z (M+H)⁺=249.1; HPLC Conditions: A; ¹H NMR (400 MHz, D₂O) δ 4.13-4.06 (m, 1H), 4.05-3.73 (m, 4H), 2.55-2.40 (m, 2H), 1.99 (quin, J=7.67 Hz, 2H), 1.43-1.32 (m, 2H), 0.73-0.62 (m, 1H), 0.45-0.38 (m, 2H), 0.02 (q, J=4.75 Hz, 2H).

### Exemplary Embodiment 1r

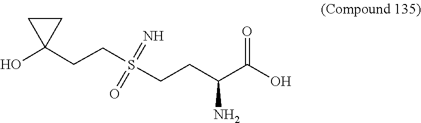 [SMILES: N=S(=O)(CC[C@H](N)C(=O)O)CCC1(O)CC1]

### (2S)-2-amino-4-(2-(1-hydroxycyclopropyl)ethylsulfonimidoyl)butanoic acid

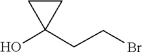

To a solution of methyl 3-bromopropanoate (1.00 g, 6 mmol, 654 mL) in THE (20 mL) was added Ti(i-PrO)₄ (170 mg, 599 mmol, 177 mL) and ethyl magnesium bromide (3 M, 4.39 mL). The mixture was stirred at 20° C. for 2 h. The reaction was quenched by NH₄C₁ (50 mL), filtered then extracted with ethyl acetate (50 mL×3). The combined organic phase was washed with brine (20 mL×2), dried over anhydrous Na₂SO₄, filtered, and concentrated. The residue was purified by prep-TLC (SiO₂, Petroleum ether: ethyl acetate=3:1, R_{f}=0.5) to give 1-(2-bromoethyl)cyclopropanol (260 mg, 26% yield) as a yellow oil. ¹H NMR (400 MHz, CDCl₃-d) δ 3.63 (t, J=7.3 Hz, 2H), 2.14 (t, J=7.3 Hz, 2H), 0.86-0.81 (m, 2H), 0.59-0.54 (m, 2H).

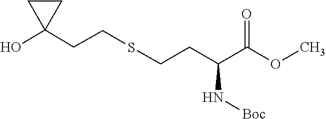 [SMILES: COC(=O)[C@H](CCSCCC1(O)CC1)NC(=O)OC(C)(C)C]

To a solution of 1-(2-bromoethyl)cyclopropanol (218 mg, 1.32 mmol) and methyl (tert-butoxycarbonyl)-L-homocysteinate (0.22 g, 882 mmol) in DMF (3 mL) was added K₂CO₃ (244 mg, 1.76 mmol). The mixture was stirred at 20° C. for 16 h under N₂. The reaction was quenched with H₂O (30 mL) and extracted with ethyl acetate (30 mL×2). The combined organic phase was dried over anhydrous Na₂SO₄, filtered, and concentrated. The residue was purified by prep-TLC (SiO₂, Petroleum ether: ethyl acetate=1:1, R_{f}=0.6) to give (S)-methyl 2-((tert-butoxycarbonyl)amino)-4-((2-(1-hydroxycyclopropyl)ethyl)thio)butanoate (270 mg, 73% yield) as colorless oil. ¹H NMR (400 MHz, CDCl₃-d) δ 5.24-5.03 (m, 1H), 4.57-4.35 (m, 1H), 3.77 (s, 3H), 2.82-2.74 (m, 2H), 2.62 (t, J=7.5 Hz, 2H), 2.21-2.08 (m, 1H), 1.99-1.78 (m, 3H), 1.46 (s, 9H), 0.83-0.76 (m, 2H), 0.55-0.42 (m, 2H).

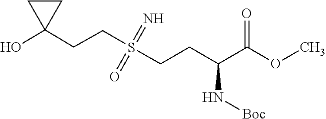 [SMILES: COC(=O)[C@H](CCS(=N)(=O)CCC1(O)CC1)NC(=O)OC(C)(C)C]

To a solution of (S)-methyl 2-((tert-butoxycarbonyl)amino)-4-((2-(1-hydroxycyclopropyl)ethyl)thio)butanoate (270 mg, 810 mmol) in MeOH (5 mL) was added ammonium carbamate (632 mg, 8.10 mmol) and PhI(OAc)₂ (1.30 g, 4.05 mmol). The mixture was stirred at 30° C. for 16 h. The mixture was concentrated to give crude product. The crude product was quenched by H₂O (20 mL) and then extracted with ethyl acetate (20 mL×4). The combined organic phase was dried over anhydrous Na₂SO₄, filtered, and concentrated. The residue was purified by prep-TLC (SiO₂, Ethyl acetate:Methanol=10:1, R_{f}=0.5) to give 2-((tert-butoxycarbonyl)amino)-4-(2-(1-hydroxycyclopropyl)ethylsulfonimidoyl)butanoate (150 mg, 51% yield) as a yellow oil. ¹H NMR (400 MHz, CDCl₃-d) δ 5.40-5.21 (m, 1H), 4.54-4.35 (m, 1H), 3.79 (s, 3H), 3.46-3.04 (m, 4H), 2.52-2.36 (m, 1H), 2.27-2.06 (m, 3H), 1.46 (s, 8H), 0.92-0.81 (m, 2H), 0.65-0.44 (m, 2H).

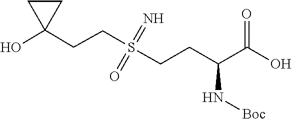 [SMILES: CC(C)(C)OC(=O)N[C@@H](CCS(=N)(=O)CCC1(O)CC1)C(=O)O]

To a solution of 2-((tert-butoxycarbonyl)amino)-4-(2-(1-hydroxycyclopropyl)ethylsulfonimidoyl)butanoate (80.0 mg, 220 mmol) in THE (2 mL) and H₂O (0.4 mL) was added LiOH·H₂O (18.4 mg, 439 mmol). The mixture was stirred at 20° C. for 2 h. The mixture was concentrated, H₂O (1 mL) was added and the aqueous phase was adjusted to pH˜6 with iN HCl. The residue was purified by prep-HPLC (column: Phenomenex Luna C18 100×30 mm, 5 microns; mobile phase: [water (0.1% formic acid)-MeCN]; B %: 10%-40%, 10 min) to give (2S)-2-((tert-butoxycarbonyl)amino)-4-(2-(1-hydroxycyclopropyl)ethylsulfonimidoyl)butanoic acid (50 mg, 65% yield) as a white solid. ¹H NMR (400 MHz, D₂O) δ 4.29 (br d, J=1.1 Hz, 1H), 3.99-3.68 (m, 4H), 2.58-2.40 (m, 1H), 2.36-2.22 (m, 1H), 2.19-2.01 (m, 2H), 1.41 (s, 9H), 0.86-0.73 (m, 2H), 0.66-0.53 (m, 2H).

To a solution of (2S)-2-((tert-butoxycarbonyl)amino)-4-(2-(1-hydroxycyclopropyl)ethylsulfonimidoyl)butanoic acid (40.0 mg, 114 mmol) in DCM (1 mL) was added TFA (0.1 mL) and the mixture was stirred at 20° C. for 1 h. The mixture was concentrated under N₂ and lyophilized to afford (2S)-2-amino-4-(2-(1-hydroxycyclopropyl)ethylsulfonimidoyl)butanoic acid (Compound 135) (35 mg, 79% yield, TFA) as a yellow oil. LCMS: Rt=0.353 min., (ES⁺) m/z (M+H)⁺=251.0; HPLC Conditions: A; ¹H NMR (400 MHz, D₂O) δ 4.13 (br t, J=6.7 Hz, 1H), 4.06-3.84 (m, 4H), 2.57-2.43 (m, 2H), 2.18-2.04 (m, 2H), 0.83-0.75 (m, 2H), 0.63-0.57 (m, 2H).

### Exemplary Embodiment 1s

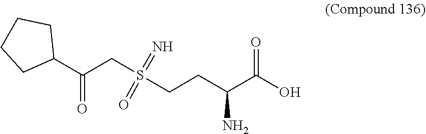 [SMILES: N=S(=O)(CC[C@H](N)C(=O)O)CC(=O)C1CCCC1]

### (2S)-2-amino-4-(2-cyclopentyl-2-oxoethylsulfonimidoyl)butanoic acid

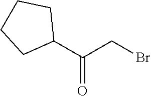

To a solution of 1-cyclopentylethanone (1.0 g, 8.92 mmol) in MeOH (10 mL) was added Br2 (1.28 g, 8.02 mmol, 414 mL) at 0° C. The mixture was stirred at 0-20° C. for 1 h. It was partially neutralized by the cautious addition of solid potassium carbonate. The mixture was poured into water (10 mL) and extracted with EtOAc (15 mL×3). The combined organic extracts were dried over Na₂SO₄, filtered, and concentrated to give 2-bromo-1-cyclopentylethanone (650 mg, 38% yield) as a colorless solid. ¹H NMR (400 MHz, CDCl₃-d) δ 3.99 (s, 2H), 3.28-3.12 (m, 1H), 1.96-1.85 (m, 2H), 1.84-1.53 (m, 8H).

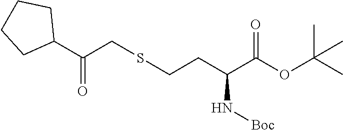 [SMILES: CC(C)(C)OC(=O)N[C@@H](CCSCC(=O)C1CCCC1)C(=O)OC(C)(C)C]

To a solution of 2-bromo-1-cyclopentylethanone (540 mg, 2.83 mmol) in DCM (7 mL) was added (S)-tert-butyl 2-((tert-butoxycarbonyl)amino)-4-mercaptobutanoate (824 mg, 2.83 mmol) and TEA (572 mg, 5.65 mmol, 787 mL) at −20° C. The mixture was stirred at 25° C. for 0.5 h under Ar. The mixture was poured into water (10 mL) and extracted with EtOAc (15 mL×3). The combined organic extracts were dried over Na₂SO₄, filtered, and concentrated. The residue was purified by column chromatography (SiO₂, Petroleum ether: ethyl acetate=90:1) to give (S)-tert-butyl 2-((tert-butoxycarbonyl)amino)-4-((2-cyclopentyl-2-oxoethyl)thio)butanoate (970 mg, 85% yield) as a colorless solid. ¹H NMR (400 MHz, CDCl₃-d) δ 5.09 (br d, J=8.1 Hz, 1H), 4.27 (br d, J=5.0 Hz, 1H), 3.30 (s, 2H), 2.61-2.47 (m, 2H), 2.19-1.99 (m, 2H), 1.93-1.79 (m, 3H), 1.78-1.54 (m, 6H), 1.51-1.42 (m, 18H).

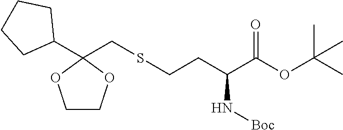 [SMILES: CC(C)(C)OC(=O)N[C@@H](CCSCC1(C2CCCC2)OCCO1)C(=O)OC(C)(C)C]

To a solution of (S)-tert-butyl 2-((tert-butoxycarbonyl)amino)-4-((2-cyclopentyl-2-oxoethyl)thio)butanoate (860 mg, 2.14 mmol) in DCM (9 mL) was added ethylene glycol (2.66 g, 42.8 mmol, 2.40 mL), trimethoxymethane (2.27 g, 21.4 mmol, 2.35 mL) and p-TsOH (36.9 mg, 214 mmol). The mixture was stirred at 20° C. for 16 h. The mixture was poured into NaHCO₃ (11 mL) and extracted with EtOAc (10 mL×3). The combined organic extracts were dried over Na₂SO₄, filtered, and concentrated. The residue was purified by column chromatography (SiO₂, Petroleum ether: ethyl acetate=92:8) to give (S)-tert-butyl 2-((tert-butoxycarbonyl)amino)-4-(((2-cyclopentyl-1,3-dioxolan-2-yl)methyl)thio)butanoate (700 mg, 74% yield) as a colorless oil. ¹H NMR (400 MHz, CDCl₃-d) δ 5.11 (br d, J=7.5 Hz, 1H), 4.26 (br d, J=4.9 Hz, 1H), 4.18-4.05 (m, 2H), 4.03-3.92 (m, 2H), 2.74 (s, 2H), 2.70-2.56 (m, 2H), 2.50 (quin, J=8.6 Hz, 1H), 2.15-2.03 (m, 1H), 1.95-1.81 (m, 1H), 1.74-1.59 (m, 5H), 1.57-1.35 (m, 20H).

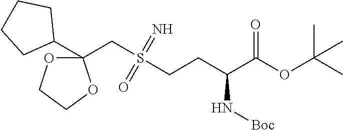 [SMILES: CC(C)(C)OC(=O)N[C@@H](CCS(=N)(=O)CC1(C2CCCC2)OCCO1)C(=O)OC(C)(C)C]

To a solution of (S)-tert-butyl 2-((tert-butoxycarbonyl)amino)-4-(((2-cyclopentyl-1,3-dioxolan-2-yl)methyl)thio)butanoate (300 mg, 673 mmol) in i-PrOH (4 mL) was added ammonium carbamate (420 mg, 5.39 mmol) and PhI(OAc)₂ (867 mg, 2.69 mmol). The mixture was stirred at 20° C. for 2 h. The mixture was poured into water (10 mL) and extracted with EtOAc (15 mL×3). The combined organic extracts were dried over Na₂SO₄, filtered, and concentrated. The residue was purified by column chromatography (SiO₂, Petroleum ether: ethyl acetate=45:55) to give (2S)-tert-butyl 2-((tert-butoxycarbonyl)amino)-4-(S-((2-cyclopentyl-1,3-dioxolan-2-yl)methyl) sulfonimidoyl)butanoate (100 mg, 32% yield) as a colorless oil. ¹H NMR (400 MHz, CDCl₃-d) δ 5.31-5.21 (m, 1H), 4.26 (br s, 1H), 4.20-4.10 (m, 2H), 4.08-3.97 (m, 2H), 3.38-3.08 (m, 2H), 2.63 (quin, J=8.4 Hz, 1H), 2.46-2.32 (m, 1H), 2.24-2.08 (m, 2H), 1.78-1.53 (m, 7H), 1.52-1.36 (m, 20H).

To a solution of (2S)-tert-butyl 2-((tert-butoxycarbonyl)amino)-4-(S-((2-cyclopentyl-1,3-dioxolan-2-yl)methyl) sulfonimidoyl)butanoate (50 mg, 105 mmol) in DCM (0.9 mL) and added HCl (12M, 0.3 mL). The mixture was stirred at 20° C. for 4 h and the residue was purified by prep-HPLC (column: Phenomenex Luna C18 100×30 mm, 5 microns; mobile phase: [water(0.04% HCl)-MeCN]; B %: 1%-25%, 10 min) to give (2S)-2-amino-4-(2-cyclopentyl-2-oxoethylsulfonimidoyl)butanoic acid (Compound 136) (8 mg, 28% yield) as a yellow solid. LCMS: Rt=1.686 min., (ES⁺) m/z (M+H)⁺=277.0; HPLC Conditions: A; ¹H NMR (400 MHz, D₂O) δ 4.20-4.08 (m, 1H), 3.95-3.71 (m, 2H), 3.09 (quin, J=7.9 Hz, 1H), 2.72-2.57 (m, 1H), 2.56-2.37 (m, 2H), 2.34-2.14 (m, 1H), 1.91-1.78 (m, 2H), 1.75-1.65 (m, 2H), 1.63-1.52 (m, 4H).

### Exemplary Embodiment 1t

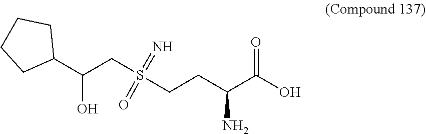 [SMILES: N=S(=O)(CC[C@H](N)C(=O)O)CC(O)C1CCCC1]

### (2S)-2-amino-4-(S-((2-cyclopentyl-1,3-dioxolan-2-yl)methyl)sulfonimidoyl)butanoic acid

To a solution of (2S)-2-amino-4-(2-cyclopentyl-2-oxoethylsulfonimidoyl)butanoic acid (10 mg, 36.2 mmol) in EtOH (1 mL) was added NaBH₄ (1.37 mg, 36.1 mmol). The mixture was stirred at 0° C. for 0.5 h. The mixture was purified by prep-HPLC (column: Phenomenex Luna C18 100×30 mm, 5 microns; mobile phase: [water (0.1% formic acid)-MeCN]; B %: 1%-30%, 10 min) to give (2S)-2-amino-4-(S-((2-cyclopentyl-1,3-dioxolan-2-yl)methyl)sulfonimidoyl)butanoic acid (Compound 137) (3.33 mg, 22% yield, TFA) as a colorless oil. LCMS: Rt=1.325 min, (ES⁺) m/z (M+H)⁺=279.0; HPLC Conditions: A; ¹H NMR (400 MHz, D₂O) δ 1.06-1.22 (m, 1H), 1.26-1.40 (m, 1H), 1.44-1.63 (m, 4H), 1.64-1.81 (m, 2H), 2.02 (sxt, J=7.92 Hz, 1H), 2.38-2.55 (m, 2H), 3.79-4.13 (m, 6H).

### Exemplary Embodiment 1u

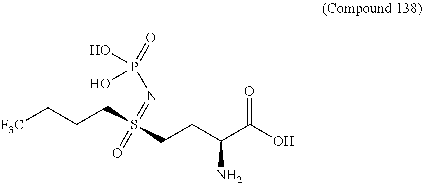 [SMILES: N[C@@H](CC[S@@](=O)(CCCC(F)(F)F)=NP(=O)(O)O)C(=O)O]

### (S)-2-amino-4-((S)-4,4,4-trifluoro-N-phosphonobutylsulfonimidoyl)butanoic acid

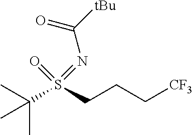 [SMILES: CC(C)(C)C(=O)N=[S@](=O)(CCCC(F)(F)F)C(C)(C)C]

To a solution of (S)—N-(tert-butylsulfinyl)pivalamide (3.00 g, 14.6 mmol) in dioxane (90 mL) was added NaH (701 mg, 17.5 mmol, 60% purity) and 15-crown-5 (3.86 g, 17.5 mmol, 3.48 mL) at 15° C. for 10 min under N₂ atmosphere. Then 1,1,1-trifluoro-4-iodobutane (9.74 g, 40.9 mmol) was added and the resulting mixture was stirred at 40° C. for 48 h. The reaction mixture was poured into ice water (50 mL) and extracted with EtOAc (25 mL×2). The combined organic extracts were washed with brine (30 mL), dried over Na₂SO₄, filtered, and concentrated. The residue was purified by column chromatography (SiO₂, Petroleum ether/Ethyl acetate=1/0 to 1/1) to give N-(tert-butyl(oxo)(4,4,4-trifluorobutyl)-l⁶-sulfaneylidene)pivalamide (3.54 g, 77% yield) as a white solid. ¹H NMR (400 MHz, CDCl₃-d) δ 3.61 (ddd, J=5.6, 8.8, 14.0 Hz, 1H), 3.44-3.30 (m, 1H), 2.44-2.07 (m, 4H), 1.48 (s, 9H), 1.20 (s, 9H).

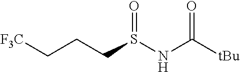 [SMILES: CC(C)(C)C(=O)N[S@](=O)CCCC(F)(F)F]

To a solution of N-(tert-butyl(oxo)(4,4,4-trifluorobutyl)-l⁶-sulfaneylidene)pivalamide (3.54 g, 11.2 mmol) in DCM (35 mL) was added TFA (1.92 g, 16.8 mmol, 1.25 mL). The mixture was stirred at 20° C. for 40 min and concentrated. The residue was diluted with an aqueous solution of NaHCO₃ (30 mL) and extracted with EtOAc (20 mL×2). The combined organic extracts were washed with brine (20 mL), dried over Na₂SO₄, filtered, and concentrated to give (R)—N-((4,4,4-trifluorobutyl)sulfinyl)pivalamide (2.70 g, 88% yield) as a white solid. ¹H NMR (400 MHz, CDCl₃-d) δ 8.29 (br s, 1H), 3.22-2.97 (m, 2H), 2.39-2.21 (m, 2H), 2.03 (quin, J=7.6 Hz, 2H), 1.24 (s, 9H).

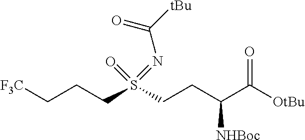 [SMILES: CC(C)(C)OC(=O)N[C@@H](CC[S@@](=O)(CCCC(F)(F)F)=NC(=O)C(C)(C)C)C(=O)OC(C)(C)C]

To a solution of (R)—N-((4,4,4-trifluorobutyl)sulfinyl)pivalamide (1.00 g, 3.86 mmol) in dioxane (30 mL) was added NaH (385 mg, 9.64 mmol, 60% in oil) and 15-crown-5 (1.70 g, 7.71 mmol, 1.53 mL) in one portion at 25° C. The mixture was stirred for 10 min and (S)-tert-butyl 2-((tert-butoxycarbonyl)amino)-4-iodobutanoate (3.71 g, 9.64 mmol) was added. The mixture was stirred at 40° C. for 144 h. The reaction mixture was poured into water (60 mL) andextracted with EtOAc (30 mL×2). The combined organic extracts were washed with brine (60 mL), dried over Na₂SO₄, filtered, and concentrated. The residue was purified by column chromatography (SiO₂, Petroleum ether/Ethyl acetate=1/0 to 1/1) and then purified by prep-HPLC (column: Phenomenex Luna C18 75×30 mm, 3 microns; mobile phase: [water(FA)-MeCN]; B %: 55%-85%, 8 min) to give (S)-tert-butyl 2-((tert-butoxycarbonyl)amino)-4-((S)-4,4,4-trifluoro-N-pivaloylbutylsulfonimidoyl)butanoate (520 mg, 26% yield) as a white solid. ¹H NMR (400 MHz, CDCl₃-d) δ 5.21 (br d, J=2.8 Hz, 1H), 4.28 (br d, J=4.0 Hz, 1H), 3.63-3.52 (m, 1H), 3.51-3.36 (m, 2H), 3.36-3.28 (m, 1H), 2.46-2.29 (m, 3H), 2.17-2.04 (m, 3H), 1.49 (s, 9H), 1.46 (s, 9H), 1.20 (s, 9H).

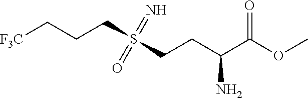 [SMILES: COC(=O)[C@@H](N)CC[S@@](=N)(=O)CCCC(F)(F)F]

(S)-tert-butyl 2-((tert-butoxycarbonyl)amino)-4-((S)-4,4,4-trifluoro-N-pivaloylbutylsulfonimidoyl)butanoate (270 mg, 523 mmol) in HCl/MeOH (5 mL) was stirred at 30° C. for 12 h. The reaction mixture was concentrated to give (S)-methyl 2-amino-4-((S)-4,4,4-trifluorobutylsulfonimidoyl)butanoate (170 mg, 99.5% yield, HCl) as a yellow oil. ¹H NMR (400 MHz, MeOD-d₄) δ 4.39-4.30 (m, 1H), 4.11-3.92 (m, 3H), 3.91 (s, 3H), 3.89-3.80 (m, 1H), 2.56-2.43 (m, 4H), 2.25-2.18 (m, 2H).

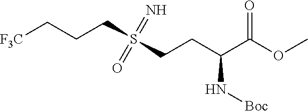 [SMILES: COC(=O)[C@H](CC[S@@](=N)(=O)CCCC(F)(F)F)NC(=O)OC(C)(C)C]

To a solution of (S)-methyl 2-amino-4-((S)-4,4,4-trifluorobutylsulfonimidoyl)butanoate (170 mg, 585 mmol) in dioxane (2 mL) and H₂O (2 mL) was added Boc₂O (153 mg, 702 mmol, 161 mL) and Na₂CO₃ (186 mg, 1.76 mmol). The mixture was stirred at 25° C. for 16 h. The reaction mixture was poured into water (10 mL) and extracted with EtOAc (5 mL×2). The combined organic extracts were washed with brine (8 mL), dried over Na₂SO₄, filtered, and concentrated. The residue was purified by column chromatography (SiO₂, Petroleum ether/Ethyl acetate=5/1 to 0/1) to give (S)-methyl 2-((tert-butoxycarbonyl)amino)-4-((S)-4,4,4-trifluorobutylsulfonimidoyl)butanoate (125 mg, 55% yield) as a yellow oil. ¹H NMR (400 MHz, CDCl₃-d) δ 5.35-5.24 (m, 1H), 4.45 (br d, J=2.8 Hz, 1H), 3.80 (s, 3H), 3.35-3.10 (m, 4H), 2.52-2.37 (m, 2H), 2.37-2.31 (m, 2H), 2.19 (br d, J=7.6 Hz, 2H), 1.46 (s, 9H).

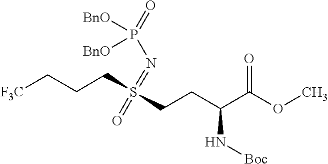 [SMILES: COC(=O)[C@H](CC[S@@](=O)(CCCC(F)(F)F)=NP(=O)(OCc1ccccc1)OCc1ccccc1)NC(=O)OC(C)(C)C]

A mixture of (S)-methyl 2-((tert-butoxycarbonyl)amino)-4-((S)-4,4,4-trifluorobutylsulfonimidoyl)butanoate (125 mg, 320 mmol), Cu(OAc)₂ (29.1 mg, 160 mmol), TEA (32.4 mg, 320 mmol, 44.5 mL) and 4 Å molecular sieves (125 mg) in toluene (3 mL) was stirred for 10 min. Dibenzyl phosphonate (184 mg, 704 mmol) was added and the resulting mixture was stirred at 110° C. for 5 h under N₂ atmosphere. The reaction mixture was filtered and concentrated. The residue was purified by column chromatography (SiO₂, Petroleum ether/Ethyl acetate=5/1 to 0/1) to give (S)-methyl 4-((S)—N-(bis(benzyloxy)phosphoryl)-4,4,4-trifluorobutylsulfonimidoyl)-2-((tert-butoxycarbonyl)amino)butanoate (75.0 mg, 36% yield) as a yellow oil. ¹H NMR (400 MHz, CDCl₃-d) δ 7.42-7.29 (m, 10H), 5.37-5.24 (m, 1H), 5.11-4.92 (m, 4H), 4.41-4.27 (m, 1H), 3.76 (s, 3H), 3.31 (br d, J=5.6 Hz, 2H), 3.27-3.16 (m, 1H), 2.48-2.33 (m, 1H), 2.23-2.07 (m, 4H), 1.74-1.59 (m, 2H), 1.45 (s, 9H).

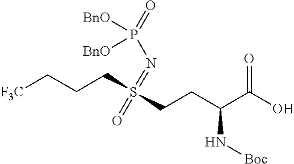 [SMILES: CC(C)(C)OC(=O)N[C@@H](CC[S@@](=O)(CCCC(F)(F)F)=NP(=O)(OCc1ccccc1)OCc1ccccc1)C(=O)O]

To a solution of (S)-methyl 4-((S)—N-(bis(benzyloxy)phosphoryl)-4,4,4-trifluorobutylsulfonimidoyl)-2-((tert-butoxycarbonyl)amino)butanoate (75 mg, 115 mmol) in THE (1.5 mL) and H₂O (0.3 mL) was added LiOH·H₂O (14.5 mg, 345 mmol). The mixture was stirred at 25° C. for 3 h then concentrated. The aqueous phase was adjusted to pH=5 with 1M HCl. The aqueous phase was extracted with DCM (3 mL×3). The combined organic extracts were dried over Na₂SO₄, filtered, and concentrated to give (S)-4-((S)—N-(bis(benzyloxy)phosphoryl)-4,4,4-trifluorobutylsulfonimidoyl)-2-((tert-butoxycarbonyl)amino)butanoic acid (73 mg, 99% yield) as a yellow oil. ¹H NMR (400 MHz, CDCl₃-d) δ 7.30 (br s, 10H), 5.75-5.59 (m, 1H), 4.96-4.83 (m, 4H), 4.37-4.27 (m, 1H), 3.74-3.33 (m, 4H), 3.12-3.00 (m, 4H), 2.38-2.25 (m, 2H), 1.20 (s, 9H).

To a solution of (S)-4-((S)—N-(bis(benzyloxy)phosphoryl)-4,4,4-trifluorobutylsulfonimidoyl)-2-((tert-butoxycarbonyl)amino)butanoic acid (68 mg, 106 mmol) in DCM (1 mL) was added TFA (1 mL) at 0° C. The mixture was stirred at 17° C. for 12 h. The reaction mixture was concentrated. The residue was purified by prep-HPLC (column: C18-1 150×30 mm, 5 microns; mobile phase: [water(FA)-MeCN]; B %: 1%-20%, 10 min) to give (S)-2-amino-4-((S)-4,4,4-trifluoro-N-phosphonobutylsulfonimidoyl)butanoic acid (Compound 138) (22.5 mg, 51% yield, TFA) as a white solid. LCMS: Rt=0.494 min, (ES⁺) m/z (M+H)⁺=357.0; HPLC Conditions: E; ¹H NMR (400 MHz, D₂O) δ 4.06 (t, J=6.4 Hz, 1H), 3.62-3.40 (m, 4H), 2.51-2.31 (m, 4H), 2.12 (quin, J=7.6 Hz, 2H).

### Exemplary Embodiment 1v

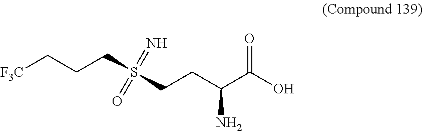 [SMILES: N=[S@](=O)(CCCC(F)(F)F)CC[C@H](N)C(=O)O]

### (S)-2-amino-4-((S)-4,4,4-trifluorobutylsulfonimidoyl)butanoic acid

A solution of (S)-tert-butyl 2-((tert-butoxycarbonyl)amino)-4-((S)-4,4,4-trifluoro-N-pivaloylbutylsulfonimidoyl)butanoate (100 mg, 193 mmol) in HCl/dioxane (4 M, 1 mL) was stirred at 25° C. for 24 h. The reaction mixture was concentrated and then added HCl (4 M, 1 mL). The mixture was stirred at 25° C. for another 24 h and concentrated. The residue was purified by prep-HPLC (column: C18-1 150×30 mm, 5 microns; mobile phase: [water(TFA)-MeCN]; B %: 1%-15%, 10 min) to give (S)-2-amino-4-((S)-4,4,4-trifluorobutylsulfonimidoyl)butanoic acid (Compound 139) (24.1 mg, 40% yield, HCl) as a white solid. LCMS: Rt=0.630 min, (ES⁺) m/z (M+H)⁺=277.0; HPLC Conditions C; ¹H NMR (400 MHz, D₂O) δ 3.99 (t, J=6.5 Hz, 1H), 3.79-3.64 (m, 1H), 3.63-3.51 (m, 3H), 2.50-2.29 (m, 4H), 2.22-2.05 (m, 2H).

### Exemplary Embodiment 1v2

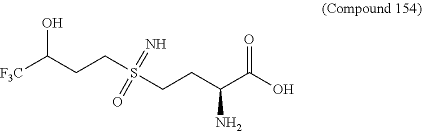 [SMILES: N=S(=O)(CCC(O)C(F)(F)F)CC[C@H](N)C(=O)O]

### (2S)-2-amino-4-(4,4,4-trifluoro-3-hydroxybutylsulfonimidoyl)butanoic acid

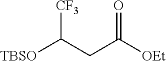 [SMILES: CCOC(=O)CC(O[Si](C)(C)C(C)(C)C)C(F)(F)F]

To a mixture of ethyl 4,4,4-trifluoro-3-hydroxy-butanoate (1.00 g, 5.37 mmol, 1 eq) and imidazole (1.10 g, 16.1 mmol, 3 eq) in DMF (10 mL) was added TBSCl (972 mg, 6.45 mmol, 790 μL, 1.2 eq) dropwise at 0° C., the mixture was stirred at 25° C. for 16 h. The mixture was quenched with water (50 mL) and extracted with MTBE (10 mL×2), the combined organic phases were dried over Na₂SO₄, filtered and the filtrate was concentrated. The residue was purified by chromatography (SiO₂, petroleum ether/EtOAc=10:1) to give ethyl 3-((tert-butyldimethylsilyl)oxy)-4,4,4-trifluorobutanoate (0.95 g, 3.16 mmol, 58.86% yield), as colorless oil. ¹H NMR (400 MHz, CDCl₃-d) δ=4.58-4.44 (m, 1H), 4.24-4.10 (m, 2H), 2.74-2.54 (m, 2H), 1.29 (t, 3H), 0.88 (s, 9H), 0.14 (s, 3H), 0.11-0.06 (m, 3H).

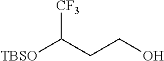 [SMILES: CC(C)(C)[Si](C)(C)OC(CCO)C(F)(F)F]

To a solution of 3-((tert-butyldimethylsilyl)oxy)-4,4,4-trifluorobutanoate (0.800 g, 2.66 mmol, 1 eq) in THF (10 mL) was added DIBALH (1 M, 6.66 mL, 2.5 eq) dropwise at −60° C., the resulting mixture was stirred at −60° C. for 3 h. The mixture was quenched with 1N HCl (5 mL) and extracted with ethyl acetate (10 mL×2), the combined organic phases were washed with brine (10 mL), dried over Na₂SO₄, filtered and concentrated to give 3-((tert-butyldimethylsilyl)oxy)-4,4,4-trifluorobutan-1-ol (0.7 g, crude), as a yellow oil. ¹H NMR (400 MHz, CDCl₃-d) δ 4.12-4.01 (m, 1H), 3.67 (br s, 2H), 1.86-1.64 (m, 2H), 1.33-1.23 (m, 1H), 0.81-0.75 (m, 9H), 0.03-0.02 (m, 6H).

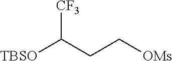 [SMILES: CC(C)(C)[Si](C)(C)OC(CCOS(C)(=O)=O)C(F)(F)F]

To a mixture of 3-((tert-butyldimethylsilyl)oxy)-4,4,4-trifluorobutan-1-ol (0.650 g, 2.52 mmol, 1 eq) and TEA (764 mg, 7.55 mmol, 1.05 mL, 3 eq) in DCM (5 mL) was added methanesulfonyl chloride (317 mg, 2.77 mmol, 214 μL, 1.1 eq) at 0° C. and stirred at 0-25° C. for 2 h. The mixture was quenched with water (10 mL) and extracted with DCM, the organic phase was dried over Na₂SO₄, filtered and concentrated to give (3-((tert-butyldimethylsilyl)oxy)-4,4,4-trifluorobutyl methanesulfonate (0.85 g, crude) as a yellow liquid. ¹H NMR (400 MHz, CDCl₃-d) δ 4.47-4.27 (m, 2H), 4.22-4.10 (m, 1H), 3.04 (s, 3H), 2.25-2.10 (m, 1H), 2.07-1.92 (m, 1H), 0.92 (s, 9H), 0.23-0.07 (m, 6H).

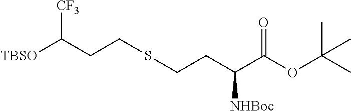 [SMILES: CC(C)(C)OC(=O)N[C@@H](CCSCCC(O[Si](C)(C)C(C)(C)C)C(F)(F)F)C(=O)OC(C)(C)C]

A mixture of (3-((tert-butyldimethylsilyl)oxy)-4,4,4-trifluorobutyl methanesulfonate (450 mg, 1.34 mmol, 1.3 eq), tert-butyl (tert-butoxycarbonyl)-L-homocysteinate (0.3 g, 1.03 mmol, 1 eq), K₂CO₃ (426.85 mg, 3.09 mmol, 3 eq) and KI (222.17 mg, 1.34 mmol, 1.3 eq) in DMF (10 mL) was heated to 45° C. for 16 h under Ar. The mixture was quenched with water (20 mL) and extracted with ethyl acetate (10 mL×2), the combined organic phases were washed with brine (20 mL), dried over Na₂SO₄, filtered and concentrated. The residue was purified by MPLC (SiO₂, petroleum ether/EtOAc=10:1) to give (2S)-tert-butyl 2-((tert-butoxycarbonyl)amino)-4-((3-((tert-butyldimethylsilyl)oxy)-4,4,4-trifluorobutyl)thio)butanoate (0.59 g, crude), as a yellow oil. ¹H NMR (400 MHz, CDCl₃-d) δ 5.15-5.05 (m, 1H), 4.28 (dt, 1H), 4.19-4.03 (m, 1H), 2.74-2.47 (m, 4H), 2.14-2.02 (m, 1H), 1.95-1.79 (m, 3H), 1.50-1.44 (m, 18H), 0.91 (s, 9H), 0.13 (d, 6H).

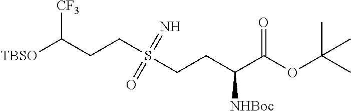 [SMILES: CC(C)(C)OC(=O)N[C@@H](CCS(=N)(=O)CCC(O[Si](C)(C)C(C)(C)C)C(F)(F)F)C(=O)OC(C)(C)C]

A mixture of (2S)-tert-butyl 2-((tert-butoxycarbonyl)amino)-4-((3-((tert-butyldimethylsilyl)oxy)-4,4,4-trifluorobutyl)thio)butanoate (0.300 g, 564 μmol, 1 eq), PhI(OAc)₂ (545 mg, 1.69 mmol, 3 eq) and ammonium carbamate (264 mg, 3.39 mmol, 6 eq) in i-PrOH (5 mL) was stirred at 25° C. for 12 h. The mixture was concentrated, the residue was added water (10 mL) and extracted with ethyl acetate (10 mL×2), the combined organic phases were dried over Na₂SO₄, filtered and concentrated. The residue was purified by column chromatography (SiO₂, petroleum ether/EtOAc=10:1) to give (2S)-tert-butyl 2-((tert-butoxycarbonyl)amino)-4-(3-((tert-butyldimethylsilyl)oxy)-4,4,4-trifluorobutylsulfonimidoyl)butanoate (0.28 g, 496.36 μmol, 87.98% yield), as a yellow oil.

¹H NMR (400 MHz, CDCl₃-d) δ 5.28-5.15 (m, 1H), 4.36-4.14 (m, 2H), 3.24-2.97 (m, 4H), 2.62-2.06 (m, 5H), 1.50-1.44 (m, 18H), 0.92 (s, 9H), 0.14 (s, 6H).

To a solution of (2S)-tert-butyl 2-((tert-butoxycarbonyl)amino)-4-(3-((tert-butyldimethylsilyl)oxy)-4,4,4-trifluorobutylsulfonimidoyl)butanoate (140 mg, 249 μmol, 1 eq) in CH₃CN (5 mL) was added concentrated HCl (0.5 mL), and the mixture stirred at 25° C. for 12 h. The mixture was concentrated, the residue was purified by prep-HPLC (column: Phenomenex C18 (75×30 mm, 3 μm); mobile phase: [water (FA)-MeCN]; gradient: 1-10% B, 12 min) to give (2S)-2-amino-4-(4,4,4-trifluoro-3-hydroxybutylsulfonimidoyl)butanoic acid (40 mg, 121.68 μmol, 48.91% yield, HCl) as white solid. LCMS: Rt=0.437 min., (ES⁺) m/z (M+H)⁺=293.0, HPLC Conditions: A. ¹H NMR (400 MHz, D₂O) δ 4.32-4.17 (m, 1H), 3.96-3.86 (m, 1H), 3.72-3.18 (m, 4H), 2.43-2.33 (m, 2H), 2.33-2.21 (m, 1H), 2.17-2.03 (m, 1H).

### Exemplary Embodiment 1v3

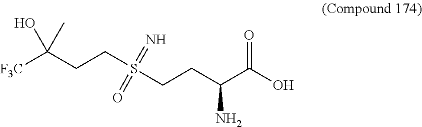 [SMILES: CC(O)(CCS(=N)(=O)CC[C@H](N)C(=O)O)C(F)(F)F]

### (2S)-2-amino-4-(4,4,4-trifluoro-3-hydroxy-3-methylbutylsulfonimidoyl)butanoic acid

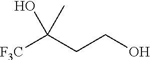

A mixture of 4,4,4-trifluoro-3-hydroxy-3-methyl-butanoic acid (500 mg, 2.91 mmol, 1 eq) in THE (6 mL) was added LiAlH₄ (165 mg, 4.36 mmol, 1.5 eq) at 0° C., and then the mixture was stirred at 20° C. for 1 h under N₂ atmosphere. The mixture was quenched with water (0.20 mL), 15% NaOH (0.20 mL) and water (0.6 mL), and the mixture was filtered, washed with ethyl acetate (5 mL×2) and the filtrate was concentrated to give 4,4,4-trifluoro-3-methylbutane-1,3-diol (300 mg, 1.90 mmol, 65.31% yield) as white oil. ¹H NMR (400 MHz, CDCl₃-d) δ 4.18-3.87 (m, 2H), 2.18-1.95 (m, 2H), 1.47-1.39 (m, 3H).

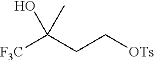 [SMILES: Cc1ccc(S(=O)(=O)OCCC(C)(O)C(F)(F)F)cc1]

To a mixture of 4,4,4-trifluoro-3-methylbutane-1,3-diol (200 mg, 1.26 mmol, 1 eq) and Et₃N (383 mg, 3.79 mmol, 528.17 μL, 3 eq) in DCM (5 mL) was added 4-methylbenzenesulfonyl chloride (241 mg, 1.26 mmol, 1 eq) at 0° C., and then the mixture was stirred at 25° C. for 1 h under N₂ atmosphere. The mixture was poured into water (6 mL) and extracted with DCM (10 mL×2). The combined organic layers were dried over Na₂SO₄, filtered, and concentrated under reduced pressure. The residue was purified by prep-TLC (petroleum ether/EtOAc=10:1) to give 4,4,4-trifluoro-3-hydroxy-3-methylbutyl 4-methylbenzenesulfonate (120 mg, 384.24 μmol, 30.38% yield) as yellow oil. ¹H NMR (400 MHz, CDCl₃-d) δ 7.81 (d, 2H), 7.37 (d, 2H), 4.39-4.17 (m, 2H), 2.47 (s, 3H), 2.12-2.02 (m, 2H), 1.39 (s, 3H).

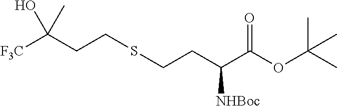 [SMILES: CC(C)(C)OC(=O)N[C@@H](CCSCCC(C)(O)C(F)(F)F)C(=O)OC(C)(C)C]

A mixture of tert-butyl (tert-butoxycarbonyl)-L-homocysteinate (123 mg, 422 μmol, 1.1 eq), 4,4,4-trifluoro-3-hydroxy-3-methylbutyl 4-methylbenzenesulfonate (120 mg, 384 μmol, 1 eq) and K₂CO₃ (159 mg, 1.15 mmol, 3 eq) in DMF (1 mL) was degassed and purged with N₂ for 3 times, and then the mixture was stirred at 40° C. for 12 h under N₂ atmosphere. The mixture was poured into water (6 mL) and extracted with EtOAc (10 mL×2). The combined organic layers were dried over Na₂SO₄, filtered, and concentrated under reduced pressure. The residue was purified by prep-TLC (petroleum ether/EtOAc=10:1) to give (2S)-tert-butyl 2-((tert-butoxycarbonyl) amino)-4-((4,4,4-trifluoro-3-hydroxy-3-methylbutyl)thio)butanoate (120 mg, 278.09 μmol, 72.37% yield) as white oil. ¹H NMR (400 MHz, CDCl₃-d) δ 5.28-5.09 (m, 1H), 4.40-4.21 (m, 1H), 2.71 (t, 2H), 2.67-2.58 (m, 2H), 2.09-2.00 (m, 2H), 1.93 (dd, 2H), 1.64-1.53 (m, 2H), 1.48 (s, 9H), 1.45 (s, 9H), 1.38 (d, 3H).

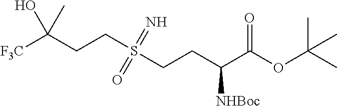 [SMILES: CC(C)(C)OC(=O)N[C@@H](CCS(=N)(=O)CCC(C)(O)C(F)(F)F)C(=O)OC(C)(C)C]

A mixture of (2S)-tert-butyl 2-((tert-butoxycarbonyl) amino)-4-((4,4,4-trifluoro-3-hydroxy-3-methylbutyl)thio)butanoate (120 mg, 324 μmol, 1 eq), ammonium carbamate (202 mg, 2.60 mmol, 8 eq) and PhI(OAc)₂ (209 mg, 648.89 μmol, 2 eq) in i-PrOH (2 mL) was stirred at 25° C. for 16 h. The mixture was poured into water (6 mL) and extracted with EtOAc (10 mL×2). The combined organic layers were dried over Na₂SO₄, filtered, and concentrated under reduced pressure. The residue was purified by prep-TLC (petroleum ether/EtOAc=1:1) to give (2S)-tert-butyl 2-((tert-butoxycarbonyl)amino)-4-(4,4,4-trifluoro-3-hydroxy-3-methylbutylsulfonimidoyl)butanoate (120 mg, 259.45 μmol, 79.97% yield), as yellow oil. ¹H NMR (400 MHz, CDCl₃-d) δ 5.66-5.45 (m, 1H), 4.29-4.18 (m, 2H), 4.01-3.87 (m, 2H), 2.62-2.49 (m, 2H), 2.40-2.32 (m, 2H), 1.50 (br s, 3H), 1.46 (s, 18H).

A mixture of (2S)-tert-butyl 2-((tert-butoxycarbonyl)amino)-4-(4,4,4-trifluoro-3-hydroxy-3-methylbutylsulfonimidoyl)butanoate (100 mg, 216.21 μmol, 1 eq) in HCl/dioxane (3 mL) was stirred at 25° C. for 12 h under N₂ atmosphere. The reaction mixture was concentrated under reduced pressure. The residue was purified by prep-HPLC (column: Phenomenex luna C18 (150×25 mm, 10 μm); mobile phase: [water (FA)-MeCN]; gradient: 0-28% B, 9 min) to give (2S′)-2-amino-4-(4,4,4-trifluoro-3-hydroxy-3-methylbutylsul fonimidoyl) butanoic acid (40.76 mg, 126.19 mol, 58.37% yield) as a white solid. LCMS: Rt=2.025 min, (ES) m/z (M+H)=307.1, HPLC Conditions: R. ¹H NMR (400 MHz, D₂O) δ 3.96 (t, 1H), 3.82-3.45 (m, 4H), 2.47-2.34 (m, 2H), 2.31-2.12 (m, 2H), 1.39 (s, 3H).

The compounds described in Table 9 were prepared using the general methods outlined above.

TABLE 9
Characterization of Compounds 140-187

| Compound | | MS | HPLC | HPLC |
|---|---|---|---|---|
| # | Structure | (M + H)+ | rt | cond. |
| 140 | 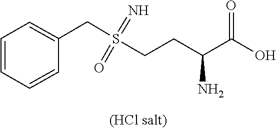 [SMILES: N=S(=O)(CC[C@H](N)C(=O)O)Cc1ccccc1] | 257.1 | 0.8 | A |
| | ¹H NMR (400 MHz, D₂O) δ 7.58-7.46 (m, | | | |
| | 5H), 5.10-4.94 (m, 2H), 4.13-4.05 (m, 1H), | | | |
| | 3.95-3.70 (m, 2H), 2.51-2.34 (m, 2H). | | | |
| 141 | 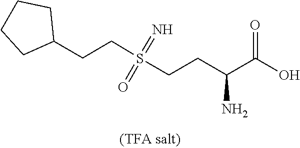 [SMILES: N=S(=O)(CCC1CCCC1)CC[C@H](N)C(=O)O] | 263.1 | 1.975 | A |
| | ¹H NMR (400 MHz, D₂O) δ 4.14-4.04 (m, | | | |
| | 1H), 4.03-3.65 (m, 4H), 2.48 (dt, 2H), 1.97- | | | |
| | 1.70 (m, 5H), 1.68-1.42 (m, 4H), 1.13 (br dd, | | | |
| | 2H). | | | |
| 142 | 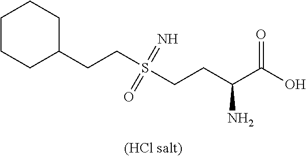 [SMILES: N=S(=O)(CCC1CCCCC1)CC[C@H](N)C(=O)O] | 277.1 | 1.648 | C |
| | ¹H NMR (400 MHz, D₂O) δ 4.10-3.98 (m, | | | |
| | 1H), 3.97-3.63 (m, 4H), 2.47-2.34 (m, 2H), | | | |
| | 1.78-1.49 (m, 7H), 1.45-1.29 (m, 1H), 1.25- | | | |
| | 0.99 (m, 3H), 0.97-0.81 (m, 2H). | | | |
| 143 | 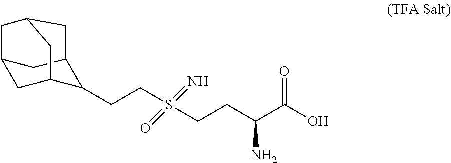 [SMILES: N=S(=O)(CCC1C2CC3CC(C2)CC1C3)CC[C@H](N)C(=O)O] | 329.1 | 1.952 | C |
| | ¹H NMR (400 MHz, D₂O) δ 4.18-4.04 (m, | | | |
| | 2H), 4.03-3.90 (m, 1H), 3.89-3.67 (m, 2H), | | | |
| | 2.67-2.28 (m, 2H), 2.01 (s, 2H), 1.91 (br s, | | | |
| | 2H), 1.70-1.54 (m, 6H), 1.47 (br s, 5H), 1.18 | | | |
| | (br t, 2H) | | | |
| 144 | 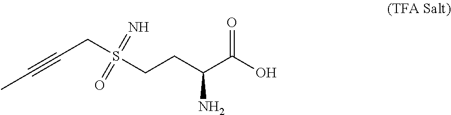 | 219.0 | 0.269 | A |
| | ¹H NMR (400 MHz, DMSO-d₆) δ 8.30 (br s, | | | |
| | 2H), 4.15 (br t, 1H), 4.09 (br d, 2H), 3.42-3.18 | | | |
| | (m, 2H), 2.26 (br dd, 2H), 1.86 (t, 3H) | | | |
| 145 | 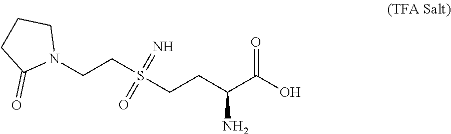 [SMILES: N=S(=O)(CC[C@H](N)C(=O)O)CCN1CCCC1=O] | 278.0 | 0.423 | A |
| | ¹H NMR (400 MHz, D₂O) δ 4.20-4.04 (m, | | | |
| | 1H), 3.96-3.63 (m, 6H), 3.50 (br t, 2H), 2.51- | | | |
| | 2.41 (m, 2H), 2.38 (br t, 2H), 2.06-1.98 (m, | | | |
| | 2H). | | | |
| 146 | 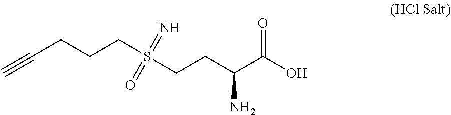 | 233.0 | 0.309 | C |
| | ¹H NMR (400 MHz, D₂O) δ 4.10-4.04 (m, | | | |
| | 1H), 4.00-3.72 (m, 4H), 2.51-2.38 (m, 5H), | | | |
| | 2.16-2.04 (m, 2H) | | | |
| 147 | 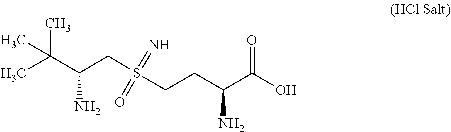 [SMILES: CC(C)(C)[C@@H](N)CS(=N)(=O)CC[C@H](N)C(=O)O] | 266.0 | 0.336 | A |
| | ¹H NMR (400 MHz, D₂O) δ 4.21-4.12 (m, | | | |
| | 1H), 3.78-3.45 (m, 5H), 2.54-2.33 (m, 2H), | | | |
| | 1.04 (s, 9H) | | | |
| 148 | 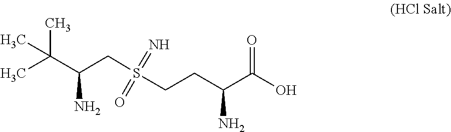 [SMILES: CC(C)(C)[C@H](N)CS(=N)(=O)CC[C@H](N)C(=O)O] | 266.2 | 0.089 | J |
| | ¹H NMR (400 MHz, D₂O) δ 4.11 (br t, 1H), | | | |
| | 3.78-3.43 (m, 5H), 2.52-2.29 (m, 2H), 1.02 | | | |
| | (s, 9H) | | | |
| 149 | 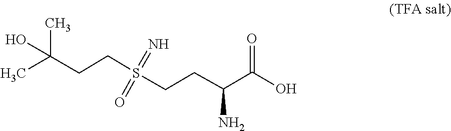 [SMILES: CC(C)(O)CCS(=N)(=O)CC[C@H](N)C(=O)O] | 253.1 | 0.296 | C |
| | ¹H NMR (400 MHz, D₂O) δ 4.13-4.03 (m, | | | |
| | 1H), 4.01-3.64 (m, 4H), 2.54-2.41 (m, 2H), | | | |
| | 2.04 (t, 2H), 1.27-1.23 (m, 6H) | | | |
| 150 | 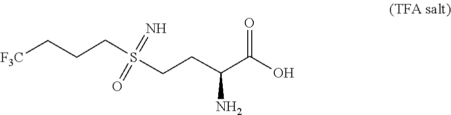 [SMILES: N=S(=O)(CCCC(F)(F)F)CC[C@H](N)C(=O)O] | 277.0 | 0.607 | E |
| | ¹H NMR (400 MHz, D₂O) δ 4.13 (br t, 1H), | | | |
| | 4.04-3.60 (m, 4H), 2.56-2.27 (m, 4H), 2.14 | | | |
| | (quin, 2H). | | | |
| 151 | 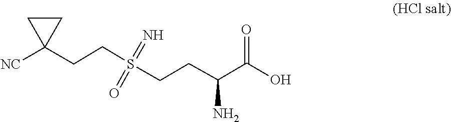 [SMILES: N#CC1(CCS(=N)(=O)CC[C@H](N)C(=O)O)CC1] | 260.0 | 0.257 | E |
| | ¹H NMR (400 MHz, MeOD) δ 4.31 (q, 1H), | | | |
| | 4.16-3.96 (m, 3H), 3.88 (ddd, 1H), 2.77-2.43 | | | |
| | (m, 3H), 2.15 (ddd, 1H), 1.71-1.61 (m, 1H), | | | |
| | 1.55-1.44 (m, 1H), 1.42-1.27 (m, 2H) | | | |
| 152 | 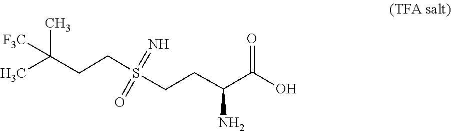 [SMILES: CC(C)(CCS(=N)(=O)CC[C@H](N)C(=O)O)C(F)(F)F] | 305.0 | 2.192 | A |
| | ¹H NMR (400 MHz, D₂O) δ 3.95-3.79 (m, | | | |
| | 1H), 3.53-3.27 (m, 4H), 2.50-2.23 (m, 2H), | | | |
| | 1.99 (br t, 2H), 1.15 (s, 6H) | | | |
| 153 | 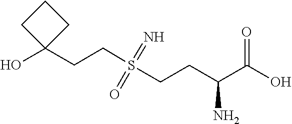 [SMILES: N=S(=O)(CC[C@H](N)C(=O)O)CCC1(O)CCC1] | 265.0 | 0.555 | A |
| | ¹H NMR (400 MHz, D₂O) δ 3.88 (dt, 1H), 3.55- | | | |
| | 3.25 (m, 4H), 2.42-2.30 (m, 2H), 2.15-2.09 | | | |
| | (m, 2H), 2.06 (dd, 4H), 1.84-1.70 (m, 1H), | | | |
| | 1.60 (td, 1H) | | | |
| 154 | 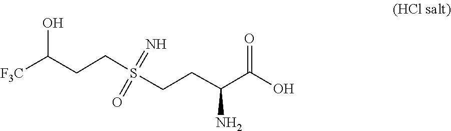 [SMILES: N=S(=O)(CCC(O)C(F)(F)F)CC[C@H](N)C(=O)O] | 293.0 | 0.437 | A |
| | ¹H NMR (400 MHz, D₂O) δ 4.32-4.17 (m, | | | |
| | 1H), 3.96-3.86 (m, 1H), 3.72-3.18 (m, 4H), | | | |
| | 2.43-2.33 (m, 2H), 2.33-2.21 (m, 1H), 2.17- | | | |
| | 2.03 (m, 1H) | | | |
| 155 | 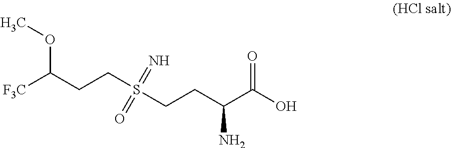 [SMILES: COC(CCS(=N)(=O)CC[C@H](N)C(=O)O)C(F)(F)F] | 307.0 | 1.56 | A |
| | ¹H NMR (400 MHz, D₂O) δ 4.04 (ddd, 1H), | | | |
| | 3.92-3.83 (m, 1H), 3.59 (s, 3H), 3.51-3.31 | | | |
| | (m, 4H), 2.42-2.22 (m, 3H), 2.16-2.02 (m, | | | |
| | 1H) | | | |
| 156 | 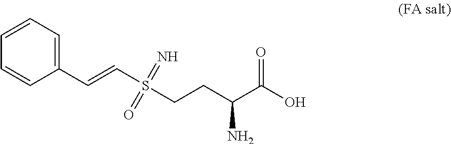 [SMILES: N=S(=O)(/C=C/c1ccccc1)CC[C@H](N)C(=O)O] | 269.0 | 1.752 | D |
| | ¹HNMR (400 MHz, D₂O) δ 7.73-7.65 (m, 2H), | | | |
| | 7.61 (d, 1H), 7.55-7.42 (m, 3H), 7.15 (d, 1H), | | | |
| | 3.89-3.77 (m, 1H), 3.55-3.34 (m, 2H), 2.41- | | | |
| | 2.21 (m, 2H). | | | |
| 157 | 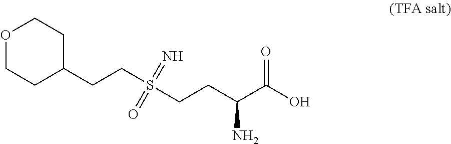 [SMILES: N=S(=O)(CCC1CCOCC1)CC[C@H](N)C(=O)O] | 279.0 | 0.695 | E |
| | ¹H NMR (400 MHz, D₂O) δ 4.17-3.53 (m, | | | |
| | 7H), 3.39 (dt, 2H), 2.72-2.35 (m, 2H), 1.89- | | | |
| | 1.57 (m, 5H), 1.35-1.13 (m, 2H). | | | |
| 158 | 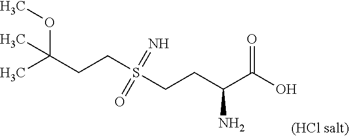 [SMILES: COC(C)(C)CCS(=N)(=O)CC[C@H](N)C(=O)O] | 267.1 | 0.915 | A |
| | ¹H NMR (400 MHz, D₂O) δ 4.17-4.11 (m, | | | |
| | 1H), 4.09-3.86 (m, 2H), 3.86-3.70 (m, 2H), | | | |
| | 3.19 (s, 3H), 2.57-2.42 (m, 2H), 2.11 (t, 2H), | | | |
| | 1.23 (s, 6H) | | | |
| 159 | 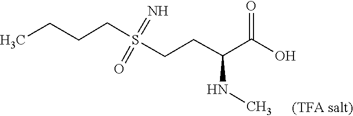 | 237.0 | 0.52 | A |
| | ¹H NMR (400 MHz, D₂O) δ 4.11-3.93 (m, | | | |
| | 1H), 3.93-3.82 (m, 2H), 3.81-3.54 (m, 2H), | | | |
| | 2.79-2.71 (m, 3H), 2.58-2.36 (m, 2H), 1.92- | | | |
| | 1.81 (m, 2H), 1.57-1.42 (m, 2H), 0.97-0.86 | | | |
| | (m, 3H) | | | |
| 160 | 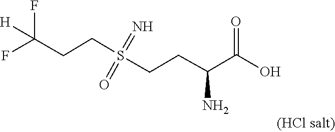 [SMILES: N=S(=O)(CCC(F)F)CC[C@H](N)C(=O)O] | 245.0 | 0.316 | E |
| | ¹H NMR (400 MHz, D₂O) δ 6.34-5.98 (m, | | | |
| | 1H), 4.12 (t, 1H), 3.89-3.64 (m, 4H), 2.60- | | | |
| | 2.36 (m, 4H) | | | |
| 161 | 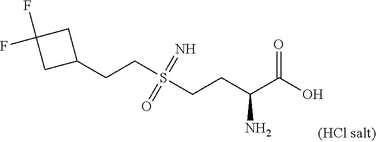 [SMILES: N=S(=O)(CCC1CC(F)(F)C1)CC[C@H](N)C(=O)O] | 285.0 | 1.016 | A |
| | ¹H NMR (400 MHz, D₂O) δ 3.86 (dt, 1H), 3.51- | | | |
| | 3.30 (m, 2H), 3.28-3.17 (m, 2H), 2.79-2.63 | | | |
| | (m, 2H), 2.41-2.16 (m, 5H), 2.06-1.92 (m, | | | |
| | 2H) | | | |
| 162 | 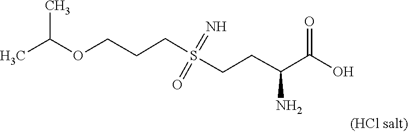 [SMILES: CC(C)OCCCS(=N)(=O)CC[C@H](N)C(=O)O] | 267 | 0.468 | F |
| | ¹H NMR (400 MHz, CDCl₃) δ 5.32-5.13 (m, | | | |
| | 1H), 4.28 (br, 1H), 3.62-3.45 (m, 3H), 3.24- | | | |
| | 2.98 (m, 4H), 2.47-2.34 (m, 1H), 2.19-2.03 | | | |
| | (m, 3H), 1.15 (d, 6H). | | | |
| 163 | 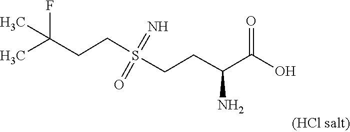 [SMILES: CC(C)(F)CCS(=N)(=O)CC[C@H](N)C(=O)O] | 255.0 | 0.602 | A |
| | ¹H NMR (400 MHz, D₂O) δ 4.05-3.96 (m, | | | |
| | 1H), 3.79-3.60 (m, 4H), 2.50-2.37 (m, 2H), | | | |
| | 2.29-2.14 (m, 2H), 1.45 (s, 3H), 1.39 (s, 3H) | | | |
| 164 | 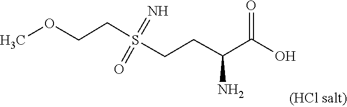 | 225.0 | 0.304 | A |
| | ¹H NMR (400 MHz, D₂O) δ 4.14-4.07 (m, | | | |
| | 1H), 4.02-3.90 (m, 6H), 3.35 (s, 3H), 2.46 (dt, | | | |
| | 2H) | | | |
| 165 | 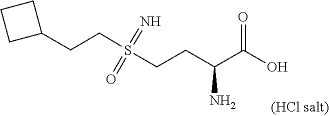 [SMILES: N=S(=O)(CCC1CCC1)CC[C@H](N)C(=O)O] | 249.0 | 0.758 | C |
| | ¹H NMR (400 MHz, D₂O) δ 4.12-4.05 (m, | | | |
| | 1H), 4.04-3.81 (m, 2H), 3.74-3.58 (m, 2H), | | | |
| | 2.55-2.44 (m, 2H), 2.43-2.35 (m, 1H), 2.12- | | | |
| | 2.03 (m, 2H), 1.98 (q, 2H), 1.92-1.75 (m, 2H), | | | |
| | 1.73-1.60 (m, 2H) | | | |
| 166 | 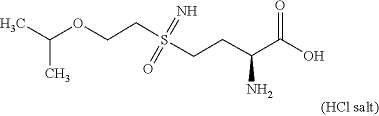 [SMILES: CC(C)OCCS(=N)(=O)CC[C@H](N)C(=O)O] | 253.2 | 0.535 | L |
| | ¹H NMR (400 MHz, D₂O) δ 4.11-3.95 (m, | | | |
| | 7H), 3.77-3.73 (m, 1H), 2.53-2.49 (m, 2H), | | | |
| | 1.16 (d, 6H). | | | |
| 167 | 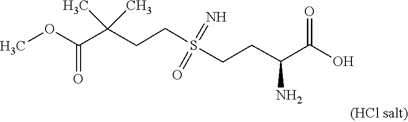 [SMILES: COC(=O)C(C)(C)CCS(=N)(=O)CC[C@H](N)C(=O)O] | 295.0 | 0.247 | H |
| | ¹H NMR (400 MHz, D₂O) δ = 3.96-3.80 (m, | | | |
| | 1H), 3.69 (s, 3H), 3.52-3.20 (m, 4H), 2.41- | | | |
| | 2.23 (m, 2H), 2.12-1.92 (m, 2H), 1.21 (s, 6H) | | | |
| 168 | 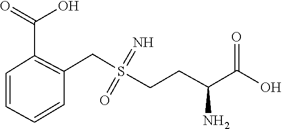 [SMILES: N=S(=O)(CC[C@H](N)C(=O)O)Cc1ccccc1C(=O)O] | 301.1 | 0.35 | G |
| | ¹H NMR (400 MHz, D₂O) δ 7.83 (br d, 1H), | | | |
| | 7.62-7.44 (m, 3H), 5.19-4.96 (m, 2H), 3.83 | | | |
| | (q, 1H), 3.52-3.20 (m, 2H), 2.45-2.21 (m, | | | |
| | 2H) | | | |
| 169 | 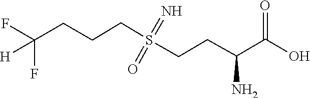 [SMILES: N=S(=O)(CCCC(F)F)CC[C@H](N)C(=O)O] | 259.0 | 0.399 | A |
| | ¹H NMR (400 MHz, D₂O) δ 6.19-5.81 (m, | | | |
| | 1H), 3.86 (dt, 1H), 3.50-3.28 (m, 4H), 2.42- | | | |
| | 2.26 (m, 2H), 2.13-1.88 (m, 4H) | | | |
| 170 | 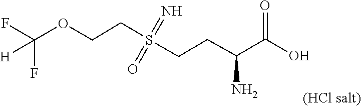 [SMILES: N=S(=O)(CCOC(F)F)CC[C@H](N)C(=O)O] | 261.0 | 0.342 | A |
| | ¹H NMR (400 MHz, D₂O) δ 6.69-6.13 (m, | | | |
| | 1H), 4.37 (br s, 2H), 4.08 (br s, 1H), 4.00-3.69 | | | |
| | (m, 4H), 2.71-2.27 (m, 2H) | | | |
| 171 | 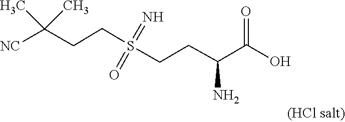 [SMILES: CC(C)(C#N)CCS(=N)(=O)CC[C@H](N)C(=O)O] | 262.0 | 0.639 | E |
| | ¹H NMR (400 MHz, D₂O) δ 4.02 (t, 1H), 3.80- | | | |
| | 3.52 (m, 4H), 2.47-2.33 (m, 2H), 2.11 (t, 2H), | | | |
| | 1.36 (s, 6H) | | | |
| 172 | 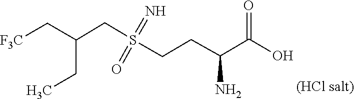 [SMILES: CCC(CC(F)(F)F)CS(=N)(=O)CC[C@H](N)C(=O)O] | 305.0 | 1.465 | C |
| | ¹H NMR (400 MHz, D₂O) δ = 4.08 (br s, 1H), | | | |
| | 3.83-3.57 (m, 4H), 2.58-2.41 (m, 5H), 1.67 | | | |
| | (br s, 2H), 0.93 (br t, 3H) | | | |
| 173 | 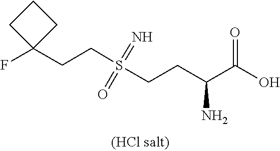 [SMILES: N=S(=O)(CC[C@H](N)C(=O)O)CCC1(F)CCC1] | 267.1 | 1.129 | A |
| | ¹H NMR (400 MHz, D₂O) δ 3.87 (dt, 1H), 3.54- | | | |
| | 3.30 (m, 4H), 2.42-2.19 (m, 6H), 2.19-2.05 | | | |
| | (m, 2H), 1.88-1.73 (m, 1H), 1.53 (m, 1H) | | | |
| 174 | 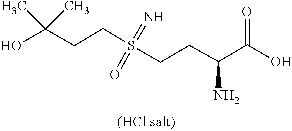 [SMILES: CC(C)(O)CCS(=N)(=O)CC[C@H](N)C(=O)O] | 307.1 | 2.025 | R |
| | ¹H NMR (400 MHz, D₂O) δ 3.96 (t, 1H), 3.82- | | | |
| | 3.45 (m, 4H), 2.47-2.34 (m, 2H), 2.31-2.12 | | | |
| | (m, 2H), 1.39 (s, 3H) | | | |
| 175 | 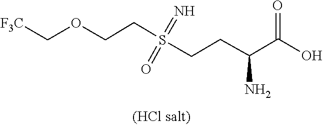 [SMILES: N=S(=O)(CCOCC(F)(F)F)CC[C@H](N)C(=O)O] | 293.0 | 2.481 | S |
| | ¹H NMR (400 MHz, D₂O) δ 4.15-4.11 (m, | | | |
| | 2H), 4.06-3.97 (m, 3H), 3.92 (br dd, 2H), 3.88- | | | |
| | 3.72 (m, 2H), 2.48-2.37 (m, 2H). | | | |
| 176 | 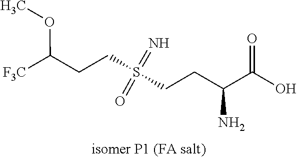 [SMILES: COC(CC[S@@](=N)(=O)CC[C@H](N)C(=O)O)C(F)(F)F] | 307.0 | 1.237 | E |
| | ¹H NMR (400 MHz, D₂O) δ 4.02-3.89 (m, | | | |
| | 1H), 3.81 (t, 1H), 3.52 (s, 3H), 3.44-3.23 (m, | | | |
| | 4H), 2.35-2.26 (m, 2H), 2.25-2.13 (m, 1H), | | | |
| | 2.10-1.94 (m, 1H). | | | |
| 177 | 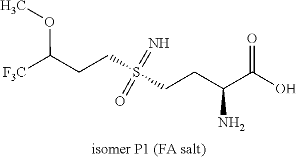 [SMILES: COC(CC[S@@](=N)(=O)CC[C@H](N)C(=O)O)C(F)(F)F] | 307.0 | 1.242 | E |
| | ¹H NMR (400 MHz, D₂O) δ 4.04-3.91 (m, | | | |
| | 1H), 3.81 (t, 1H), 3.52 (s, 3H), 3.45-3.24 (m, | | | |
| | 4H), 2.34-2.26 (m, 2H), 2.25-2.15 (m, 1H), | | | |
| | 2.09-1.95 (m, 1H). | | | |
| 178 | 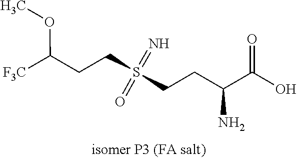 [SMILES: COC(CC[S@](=N)(=O)CC[C@H](N)C(=O)O)C(F)(F)F] | 307.0 | 1.219 | E |
| | ¹H NMR (400 MHz, D₂O) δ 4.05-3.91 (m, | | | |
| | 1H), 3.80 (t, 1H), 3.52 (s, 3H), 3.48-3.24 (m, | | | |
| | 4H), 2.39-2.26 (m, 2H), 2.25-2.14 (m, 1H), | | | |
| | 2.09-1.95 (m, 1H). | | | |
| 179 | 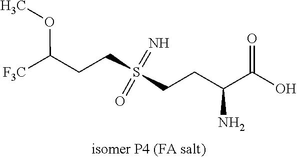 [SMILES: COC(CC[S@](=N)(=O)CC[C@H](N)C(=O)O)C(F)(F)F] | 307.0 | 1.174 | E |
| | ¹H NMR (400 MHz, D₂O) δ 4.05-3.91 (m, | | | |
| | 1H), 3.80 (t, 1H), 3.52 (s, 3H), 3.48-3.23 (m, | | | |
| | 4H), 2.29 (q, 2H), 2.25-2.15 (m, 1H), 2.11- | | | |
| | 1.97 (m, 1H). | | | |
| 180 | 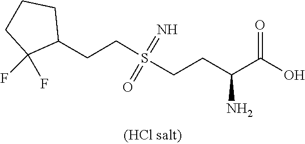 [SMILES: N=S(=O)(CCC1CCCC1(F)F)CC[C@H](N)C(=O)O] | 299.0 | 1.804 | E |
| | ¹H NMR (400 MHz, D2O) δ 4.00 (t, 1H), 3.93- | | | |
| | 3.62 (m, 4H), 2.42-2.28 (m, 2H), 2.26-2.08 | | | |
| | (m, 1H), 2.05-1.81 (m, 5H), 1.66-1.50 (m, | | | |
| | 2H), 1.35-1.26 (m, 1H) | | | |
| 181 | 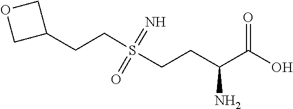 [SMILES: N=S(=O)(CCC1COC1)CC[C@H](N)C(=O)O] | 251.1 | 0.27 | C |
| | ¹H NMR (400 MHz, D₂O) δ 4.81 (br d, 2H), | | | |
| | 4.45-4.36 (m, 2H), 3.82-3.72 (m, 1H), 3.46- | | | |
| | 3.23 (m, 3H), 3.20-3.07 (m, 2H), 2.26 (br d, | | | |
| | 2H), 2.22-2.09 (m, 2H) | | | |
| 182 | 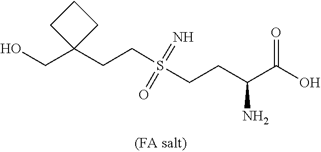 [SMILES: N=S(=O)(CC[C@H](N)C(=O)O)CCC1(CO)CCC1] | 279.1 | 1..182 | A |
| | ¹H NMR (400 MHz, D₂O) δ 3.83 (dt, 1H), 3.50 | | | |
| | (s, 2H), 3.46-3.24 (m, 2H), 3.22-3.14 (m, | | | |
| | 2H), 2.36-2.27 (m, 2H), 1.97-1.89 (m, 2H), | | | |
| | 1.87-1.69 (m, 6H) | | | |
| 183 | 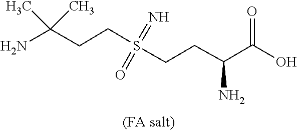 [SMILES: CC(C)(N)CCS(=N)(=O)CC[C@H](N)C(=O)O] | 252.1 | 0.267 | A |
| | ¹H NMR (400 MHz, D₂O) δ 8.58 (br d, 1H), | | | |
| | 8.45-8.40 (m, 1H), 3.86 (br s, 1H), 3.38 (br s, | | | |
| | 4H), 2.46-2.05 (m, 4H), 1.38 (br s, 6H). | | | |
| 184 | 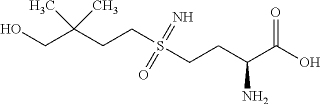 [SMILES: CC(C)(CO)CCS(=N)(=O)CC[C@H](N)C(=O)O] | 267.0 | 0.199 | H |
| | ¹H NMR (400 MHz, D₂O) δ 4.11-3.99 (m, | | | |
| | 1H), 3.97-3.77 (m, 2H), 3.75-3.56 (m, 2H), | | | |
| | 3.28 (s, 2H), 2.54-2.32 (m, 2H), 1.77 (t, 2H), | | | |
| | 0.85 (s, 6H) | | | |
| 185 | 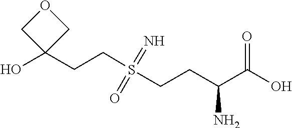 [SMILES: N=S(=O)(CC[C@H](N)C(=O)O)CCC1(O)COC1] | 265.1 | 0.546 | K |
| | ¹H NMR (400 MHz, D₂O) δ 4.53-4.47 (m, | | | |
| | 2H), 4.46-4.41 (m, 2H), 3.81-3.69 (m, 1H), | | | |
| | 3.46-3.32 (m, 2H), 3.30-3.20 (m, 2H), 2.34- | | | |
| | 2.21 (m, 2H), 2.19-2.09 (m, 2H), 1.30 (s, 3H) | | | |
| 186 | 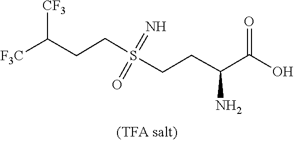 [SMILES: N=S(=O)(CCC(C(F)(F)F)C(F)(F)F)CC[C@H](N)C(=O)O] | 345.0 | 2.056 | A |
| | ¹H NMR (400 MHz, D₂O) δ 4.24-4.08 (m, | | | |
| | 1H), 3.97-3.68 (m, 5H), 2.60-2.36 (m, 4H) | | | |
| 187 | 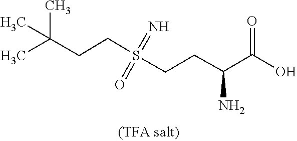 [SMILES: CC(C)(C)CCS(=N)(=O)CC[C@H](N)C(=O)O] | 251.1 | 1.798 | A |
| | ¹H NMR (400 MHz, D₂O) δ 4.19-4.10 (m, | | | |
| | 1H), 4.09-3.72 (m, 4H), 2.58-2.45 (m, 2H), | | | |
| | 1.78 (t, 2H), 0.97-0.90 (m, 9H) | | | |

### Exemplary Embodiment 1w

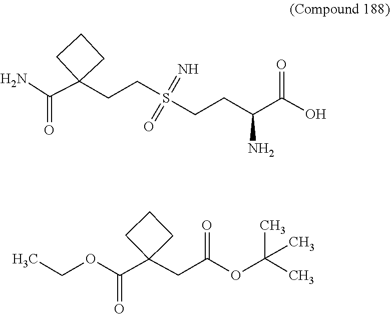 [SMILES: N=S(=O)(CC[C@H](N)C(=O)O)CCC1(C(N)=O)CCC1 ; CCOC(=O)C1(CC(=O)OC(C)(C)C)CCC1]

To a mixture of n-BuLi (2.5 M, 40 mL) in THF (400 mL) was added N-isopropylpropan-2-amine (10.1 g, 100 mmol, 14.2 mL) at −65° C. under Ar and the mixture was stirred at 0° C. for 0.5 h. The mixture was cooled to −65° C., and ethyl cyclobutanecarboxylate (12.0 g, 93.6 mmol, 12.9 mL) was added, the mixture was stirred at −65° C. for 10 min. Tert-butyl 2-bromoacetate (18.5 g, 95.0 mmol, 14.0 mL) was added and the mixture was stirred at −65° C. for 1 h. The mixture was added to sat. NH₄Cl (200 mL) at 0° C. and extracted with ethyl acetate (200 mL×2). The combined organic phases were washed with brine (200 mL×2), dried with anhydrous Na₂SO₄, filtered, and concentrated. The residue was purified by silica gel chromatography (petroleum ether/ethyl acetate=100/1 to 10/1) to give ethyl 1-(2-(tert-butoxy)-2-oxoethyl)cyclobutanecarboxylate (10 g, 44% yield) as colorless oil. ¹H NMR (400 MHz, CDCl₃-d) δ 4.16 (q, J=7.2 Hz, 2H), 2.77 (s, 2H), 2.55-2.42 (m, 2H), 2.02-1.87 (m, 4H), 1.41 (s, 9H), 1.26 (t, J=7.1 Hz, 3H).

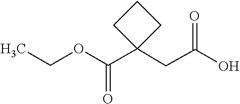

To a mixture of ethyl 1-(2-(tert-butoxy)-2-oxoethyl)cyclobutanecarboxylate (8.0 g, 33 mmol) in DCM (40 mL) was added TFA (30.8 g, 270 mmol, 20.0 mL) at 25° C. The mixture was stirred at 25° C. for 2 h and concentrated. The residue was purified by silica gel chromatography (petroleum ether/ethyl acetate=100/1 to 10/1) to give 2-(1-(ethoxycarbonyl)cyclobutyl)acetic acid (5 g, 81% yield) as yellow oil. ¹H NMR (400 MHz, CDCl₃-d) δ 4.18 (q, J=7.1 Hz, 2H), 2.90 (s, 2H), 2.64-2.45 (m, 2H), 2.10-1.90 (m, 4H), 1.26 (t, J=7.2 Hz, 3H).

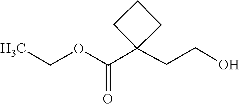

To a mixture of 2-(1-(ethoxycarbonyl)cyclobutyl)acetic acid (500 mg, 2.69 mmol) in THE (5 mL) was added BH₃·THF (1 M, 5.37 mL) at 0° C. and the mixture was stirred at 25° C. for 3 h. MeOH (1 mL) was added to the mixture and H₂O (50 mL) was added and the aqueous phase was extracted with ethyl acetate (50 mL×2). The combined organic phase was washed with brine (50 mL), dried with anhydrous Na₂SO₄, filtered, and concentrated. The residue was purified by silica gel chromatography (petroleum ether/ethyl acetate=100/1, 1/1) to give ethyl 1-(2-hydroxyethyl)cyclobutanecarboxylate (0.22 g, 48% yield) as colorless oil. ¹H NMR (400 MHz, CDCl₃-d) δ 4.17 (q, J=7.2 Hz, 2H), 3.66 (t, J=6.5 Hz, 2H), 2.53-2.42 (m, 2H), 2.08 (t, J=6.5 Hz, 2H), 2.03-1.84 (m, 4H), 1.28 (t, J=7.2 Hz, 3H).

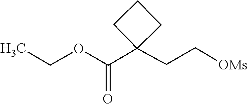 [SMILES: CCOC(=O)C1(CCOS(C)(=O)=O)CCC1]

To a mixture of ethyl 1-(2-hydroxyethyl)cyclobutanecarboxylate (220 mg, 1.28 mmol) in DCM (5 mL) at 0° C. was added TEA (388 mg, 3.83 mmol, 533 mL) and MsCl (293 mg, 2.55 mmol, 198 mL). The mixture was stirred at 25° C. for 1 h, quenched with H₂O (50 mL) and the aqueous phase was extracted with DCM (50 mL×2). The combined organic phase was washed with brine (50 mL), dried with anhydrous Na₂SO₄, filtered, and concentrated to give ethyl 1-(2-((methylsulfonyl)oxy)ethyl)cyclobutanecarboxylate (0.3 g, crude) as yellow oil.

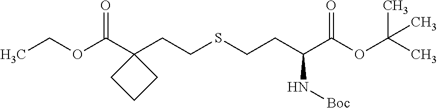 [SMILES: CCOC(=O)C1(CCSCC[C@H](NC(=O)OC(C)(C)C)C(=O)OC(C)(C)C)CCC1]

To a mixture of ethyl 1-(2-((methylsulfonyl)oxy)ethyl)cyclobutanecarboxylate (258 mg, 1.03 mmol) in DMF (5 mL) was added tert-butyl (2S)-2-(tert-butoxycarbonylamino)-4-sulfanyl-butanoate (300 mg, 1.03 mmol), KI (256 mg, 1.54 mmol) and K₂CO₃ (285 mg, 2.06 mmol) at 25° C. in a glove box. The mixture was stirred at 25° C. for 16 h. The mixture was added H₂O (50 mL) and extracted with ethyl acetate (50 mL×2). The combined organic phase was washed with brine (50 mL), dried with anhydrous Na₂SO₄, filtered and concentrated. The residue was purified by silica gel chromatography (petroleum ether/ethyl acetate=100/1 to 10/1) to give (S)-ethyl 1-(2-((4-(tert-butoxy)-3-((tert-butoxycarbonyl)amino)-4-oxobutyl)thio)ethyl) cyclobutanecarboxylate (0.25 g, 55% yield) as colorless oil. ¹H NMR (400 MHz, CDCl₃-d) δ 5.11 (br d, J=7.0 Hz, 1H), 4.27 (br d, J=4.6 Hz, 1H), 4.16 (q, J=7.1 Hz, 2H), 2.60-2.51 (m, 2H), 2.49-2.32 (m, 4H), 2.12-1.99 (m, 3H), 1.98-1.81 (m, 5H), 1.46 (d, J=9.8 Hz, 18H), 1.27 (t, J=7.2 Hz, 3H).

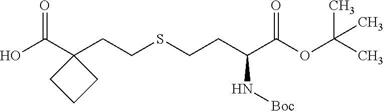 [SMILES: CC(C)(C)OC(=O)N[C@@H](CCSCCC1(C(=O)O)CCC1)C(=O)OC(C)(C)C]

(S)-ethyl 1-(2-((4-(tert-butoxy)-3-((tert-butoxycarbonyl)amino)-4-oxobutyl)thio)ethyl)cyclobutanecarboxylate (1.0 g, 2.24 mmol) in MeCN (10 mL) was added to phosphate buffer (90 mL), then pig liver esterase (900 mg) was added at 25° C., the mixture was stirred at 30° C. for 20 h. Additional pig liver esterase (200 mg) was added, and the mixture was stirred at 30° C. for another 20 h. The mixture was extracted with EtOAc (100 mL×2), dried over Na₂SO₄, filtered, and concentrated. The residue was purified by silica gel chromatography (Petroleum ether/Ethyl acetate=10/1, 5/1) to give (S)-1-(2-((4-(tert-butoxy)-3-((tert-butoxycarbonyl)amino)-4-oxobutyl)thio)ethyl)cyclobutanecarboxylic acid (500 mg, 53% yield) as colorless oil. ¹H NMR (400 MHz, CDCl₃-d) δ 5.28-5.10 (m, 1H), 4.29-4.18 (m, 1H), 2.57 (br t, J=7.9 Hz, 2H), 2.52-2.40 (m, 4H), 2.14-2.06 (m, 2H), 2.06-1.84 (m, 6H), 1.47 (d, J=7.0 Hz, 18H).

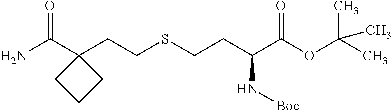 [SMILES: CC(C)(C)OC(=O)N[C@@H](CCSCCC1(C(N)=O)CCC1)C(=O)OC(C)(C)C]

To a solution of (S)-1-(2-((4-(tert-butoxy)-3-((tert-butoxycarbonyl)amino)-4-oxobutyl)thio)ethyl)cyclobutanecarboxylic acid (100 mg, 239 mmol) in DMF (3 mL) at 15° C. was added DIEA (92.9 mg, 718 mmol), HATU (182 mg, 479 mmol) and NH₄Cl (25.6 mg, 479 mmol). The mixture was stirred at 15° C. for 16 h. The mixture was added to H₂O (20 mL) and extracted with ethyl acetate (20 mL×2). The combined organic phase was washed with brine (20 mL×2), dried with anhydrous Na₂SO₄, filtered, and concentrated to give (S)-tert-butyl 2-((tert-butoxycarbonyl)amino)-4-((2-(1-carbamoylcyclobutyl)ethyl)thio)butanoate (0.1 g, crude) as red oil. ¹H NMR (400 MHz, CDCl₃-d) δ 5.78-5.58 (m, 1H), 5.48-5.27 (m, 1H), 5.23-5.01 (m, 1H), 4.38-4.21 (m, 1H), 2.65-2.36 (m, 6H), 2.05 (br t, J=8.1 Hz, 3H), 1.99-1.86 (m, 5H), 1.47 (d, J=10.0 Hz, 18H). (2S)-2-amino-4-(2-(1-carbamoylcyclobutyl)ethylsulfonimidoyl)butanoic acid: A solution of (S)-tert-butyl 2-((tert-butoxycarbonyl)amino)-4-((2-(1-carbamoylcyclobutyl)ethyl)thio)butanoate (50 mg, 77.2 μmol) in HCl/dioxane (4 M, 10 mL) was stirred at 15° C. for 16 h. The mixture was concentrated and the residue was purified by prep-HPLC: (column: C18-1 150×30 mm, 5 microns; mobile phase: [water(NH4HCO3)-ACN]; B %: 1%-10%, 10 min) to give 2S)-2-amino-4-(2-(1-carbamoylcyclobutyl)ethylsulfonimidoyl)butanoic acid (Compound 188) (3.6 mg, 15% yield), as a white solid.

LCMS: Rt=0.641 min., (ES⁺) m/z (M+H)⁺=292.1; ¹H NMR (400 MHz, D₂O) δ 3.84-3.68 (m, 1H), 3.51-3.23 (m, 2H), 3.20-3.07 (m, 2H), 2.40-2.20 (m, 6H), 2.01-1.87 (m, 3H), 1.85-1.76 (m, 1H).

The compounds described in Table 10 were prepared using the general methods outlined above.

TABLE 10
Characterization of Compounds 189-196

| Compound | | MS | HPLC | HPLC |
|---|---|---|---|---|
| # | Structure | (M + H)+ | rt | cond. |
| 189 | 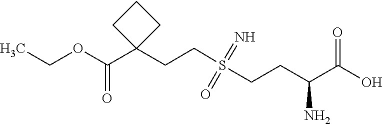 [SMILES: CCOC(=O)C1(CCS(=N)(=O)CC[C@H](N)C(=O)O)CCC1] | 321.0 | 2.471 | A |
| | ¹H NMR (400 MHz, D₂O) δ 4.18 (q, 2H), 3.85 | | | |
| | (dt, 1H), 3.51-3.27 (m, 2H), 3.22-3.14 (m, | | | |
| | 2H), 2.47-2.37 (m, 2H), 2.36-2.24 (m, 4H), | | | |
| | 2.01-1.87 (m, 4H), 1.25 (t, 3H). | | | |
| 190 | 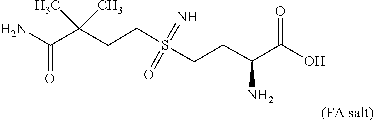 [SMILES: CC(C)(CCS(=N)(=O)CC[C@H](N)C(=O)O)C(N)=O] | 280.2 | 0.384 | A |
| | ¹H NMR (400 MHz, MeOD) δ 4.21 (br s, 1H), | | | |
| | 3.81-3.59 (m, 2H), 3.56-3.39 (m, 2H), 2.61- | | | |
| | 2.35 (m, 2H), 2.13-1.96 (m, 2H), 1.30 (s, 6H) | | | |
| 191 | 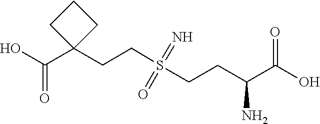 [SMILES: N=S(=O)(CC[C@H](N)C(=O)O)CCC1(C(=O)O)CCC1] | 293.0 | 1.55 | A |
| | ¹H NMR (400 MHz, D₂O) δ 3.80 (br s, 1H), | | | |
| | 3.50-3.19 (m, 2H), 3.14-2.98 (m, 2H), 2.41- | | | |
| | 2.20 (m, 4H), 2.17-2.07 (m, 2H), 1.90-1.67 | | | |
| | (m, 4H). | | | |
| 192 | 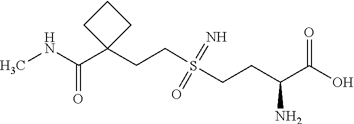 [SMILES: CNC(=O)C1(CCS(=N)(=O)CC[C@H](N)C(=O)O)CCC1] | 306.1 | 1.035 | A |
| | ¹H NMR (400 MHz, D₂O) δ 3.83-3.74 (m, | | | |
| | 1H), 3.47-3.22 (m, 2H), 3.13-3.04 (m, 2H), | | | |
| | 2.70 (s, 3H) 2.37-2.13 (m, 6H), 2.01-1.70 | | | |
| | (m, 4H). | | | |
| 193 | 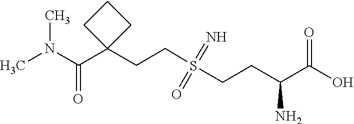 [SMILES: CN(C)C(=O)C1(CCS(=N)(=O)CC[C@H](N)C(=O)O)CCC1] | 320.1 | 0.745 | C |
| | ¹H NMR (400 MHz, D₂O) δ 3.86-3.79 (m, | | | |
| | 1H), 3.50-3.25 (m, 2H), 3.14-3.06 (m, 2H), | | | |
| | 2.92 (s, 3H), 2.85 (s, 3H), 2.52-2.39 (m, 2H), | | | |
| | 2.35-2.25 (m, 4H), 1.94 (br d, 2H), 1.75-1.64 | | | |
| | (m, 1H), 1.23 (br s, 1H). | | | |
| 194 | 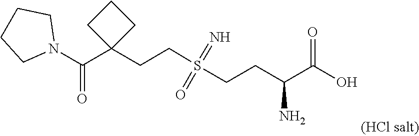 [SMILES: N=S(=O)(CC[C@H](N)C(=O)O)CCC1(C(=O)N2CCCC2)CCC1] | 346.1 | 1.44 | C |
| | ¹H NMR (400 MHz, D₂O) δ 3.85 (dt, 1H), 3.38 | | | |
| | (br d, 6H), 3.17-3.07 (m, 2H), 2.51-2.41 (m, | | | |
| | 2H), 2.41-2.31 (m, 2H), 2.31-2.26 (m, 2H), | | | |
| | 2.00-1.85 (m, 5H), 1.85-1.78 (m, 2H), 1.77- | | | |
| | 1.68 (m, 1H). | | | |
| 195 | 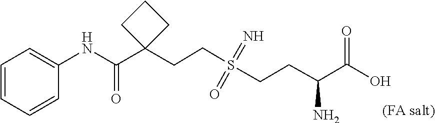 [SMILES: N=S(=O)(CC[C@H](N)C(=O)O)CCC1(C(=O)Nc2ccccc2)CCC1] | 368.1 | 2.577 | E |
| | ¹H NMR (400 MHz, D₂O) δ 7.49-7.34 (m, | | | |
| | 4H), 7.32-7.20 (m, 1H), 3.91-3.78 (m, 1H), | | | |
| | 3.55-3.28 (m, 2H), 3.26-3.13 (m, 2H), 2.49 | | | |
| | (br d, 2H), 2.41-2.23 (m, 4H), 2.11-1.94 (m, | | | |
| | 3H), 1.92-1.77 (m, 1H) | | | |
| 196 | 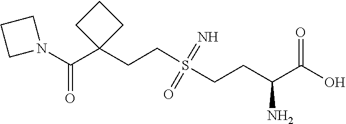 [SMILES: N=S(=O)(CC[C@H](N)C(=O)O)CCC1(C(=O)N2CCC2)CCC1] | 332.1 | 1.973 | A |
| | ¹H NMR (400 MHz, D₂O) δ 4.27-4.21 (m, | | | |
| | 2H), 3.98 (br t, 2H), 3.69-3.62 (m, 1H), 3.43- | | | |
| | 3.25 (m, 2H), 3.15-3.08 (m, 2H), 2.43-2.34 | | | |
| | (m, 2H), 2.29-2.16 (m, 6H), 2.00-1.91 (m, | | | |
| | 1H), 1.86-1.79 (m, 2H), 1.77-1.68 (m, 1H) | | | |

### Exemplary Embodiment 1x

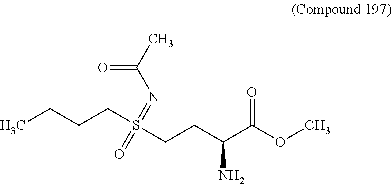 [SMILES: CCCCS(=O)(CC[C@H](N)C(=O)OC)=NC(C)=O]

### (2S)-methyl 4-(N-acetylbutylsulfonimidoyl)-2-aminobutanoate

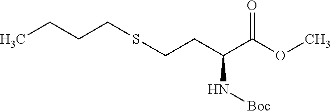 [SMILES: CCCCSCC[C@H](NC(=O)OC(C)(C)C)C(=O)OC]

To a solution of (S)-methyl 2-((tert-butoxycarbonyl)amino)-4-mercaptobutanoate (4.3 g, 17.2 mmol) and 1-bromobutane (2.84 g, 20.7 mmol, 2.23 mL) in DMF (40 mL) was added K₂CO₃ (4.77 g, 34.4 mmol). The mixture was stirred at 15° C. for 1 h. The solution was adjusted to pH˜7 with aqueous citric acid and extracted with MTBE (100 mL×2). The combined organic extracts were dried over Na₂SO₄, filtered, and concentrated to give a residue. The residue was purified by column chromatography (SiO₂, Petroleum ether: ethyl acetate=90:10) to give (S)-methyl 2-((tert-butoxycarbonyl)amino)-4-(butylthio)butanoate (4.3 g, 82% yield) as a colorless oil. ¹H NMR (400 MHz, CDCl₃-d) δ 5.11 (br s, 1H), 4.55-4.25 (m, 1H), 3.76 (s, 3H), 2.59-2.47 (m, 4H), 2.12 (br d, J=7.6 Hz, 1H), 1.92 (qd, J=7.4, 14.4 Hz, 1H), 1.61-1.55 (m, 2H), 1.48-1.36 (m, 11H), 0.92 (t, J=7.3 Hz, 3H).

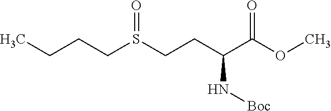 [SMILES: CCCCS(=O)CC[C@H](NC(=O)OC(C)(C)C)C(=O)OC]

To a solution of (S)-methyl 2-((tert-butoxycarbonyl)amino)-4-(butylthio)butanoate (3.5 g, 11.4 mmol) in DCM (40 mL) was added m-CPBA (2.33 g, 11.4 mmol, 85%). The mixture was stirred at 20° C. for 1 h. The reaction was quenched by NaHCO₃ (50 mL) and then extracted with DCM (50 mL×2). The combined organic phase was washed with Na₂SO₃ (20 mL), dried over anhydrous Na₂SO₄, filtered, and concentrated. The residue was purified by column chromatography (SiO₂, Petroleum ether: ethyl acetate=10:90 to 1:99) to give (2S)-methyl 2-((tert-butoxycarbonyl)amino)-4-(butylsulfinyl)butanoate (2.1 g, 57% yield) as a yellow oil. ¹H NMR (400 MHz, CDCl₃-d) δ 5.38-5.17 (m, 1H), 4.44 (br s, 1H), 3.82-3.72 (m, 3H), 2.84-2.58 (m, 4H), 2.38 (br dd, J=5.1, 12.9 Hz, 1H), 2.20-2.07 (m, 1H), 1.83-1.70 (m, 4H), 1.46 (s, 9H), 0.98 (t, J=7.3 Hz, 3H).

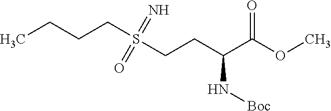 [SMILES: CCCCS(=N)(=O)CC[C@H](NC(=O)OC(C)(C)C)C(=O)OC]

To a solution of (2S)-methyl 2-((tert-butoxycarbonyl)amino)-4-(butylsulfinyl)butanoate (2.3 g, 7.16 mmol) in MeOH (25 mL) was added PhI(OAc)₂ (6.91 g, 21.4 mmol) and ammonium carbamate (2.79 g, 35.7 mmol). The mixture was stirred at 25° C. for 1 h. The solution was added H₂O (10 mL) and extracted with EtOAc (100 mL×2). The combined organic extracts were dried over Na₂SO₄, filtered, and concentrated. The residue was purified by column chromatography (SiO₂, Petroleum ether: ethyl acetate=9:91) to give (2S)-methyl 2-((tert-butoxycarbonyl)amino)-4-(butylsulfonimidoyl)butanoate (1.60 g, 66% yield) as a yellow oil. ¹H NMR (400 MHz, DMSO-d₆) δ 7.40 (br d, J=7.8 Hz, 1H), 4.16-4.07 (m, 1H), 3.64 (s, 4H), 3.13-2.86 (m, 4H), 2.17-2.05 (m, 1H), 1.91 (br s, 1H), 1.75-1.53 (m, 2H), 1.46-1.29 m 11H), 0.89 (t, J=7.3 Hz 3H).

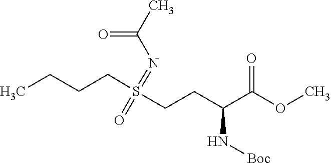 [SMILES: CCCCS(=O)(CC[C@H](NC(=O)OC(C)(C)C)C(=O)OC)=NC(C)=O]

To a solution of (2S)-methyl 2-((tert-butoxycarbonyl)amino)-4-(butylsulfonimidoyl)butanoate (0.100 g, 297 mmol) and acetyl chloride (30.3 mg, 386 mmol, 27.6 mL) in DCM (2 mL) was added TEA (90.2 mg, 891.6 mmol, 124 uL). The mixture was stirred at 15° C. for 2 h. The reaction was quenched by H₂O (10 mL) and then extracted with DCM (10 mL×2). The combined organic phase was dried over anhydrous Na₂SO₄, filtered, and concentrated to give (2S)-methyl 4-(N-acetylbutylsulfonimidoyl)-2-((tert-butoxycarbonyl)amino)butanoate (105 mg, 93% yield) as a yellow oil. ¹H NMR (400 MHz, DMSO-d₆) δ 7.50-7.23 (m, 1H), 4.24-4.08 (m, 1H), 3.65 (s, 3H), 3.54-3.37 (m, 4H), 2.20-1.98 (m, 2H), 1.92 (s, 3H), 1.73-1.58 (m, 2H), 1.44-1.31 (m, 11H), 0.90 (t, J=7.3 Hz, 3H).

To a solution of (2S)-methyl 4-(N-acetylbutylsulfonimidoyl)-2-((tert-butoxycarbonyl)amino)butanoate (70 mg, 184.9 mmol) in DCM (1 mL) was added TFA (0.1 mL). The mixture was stirred at 15° C. for 16 h. Toluene (10 mL) was added, the mixture was concentrated at 20° C. Water (10 mL) was added and the mixture was extracted with DCM (10 mL×3). The aqueous phase was lyophilized to give (2S)-methyl 4-(N-acetylbutylsulfonimidoyl)-2-aminobutanoate (Compound 197) (53 mg, 65% yield, TFA) as a colorless oil. LCMS: Rt=2.094 min., (ES⁺) m/z (M+H)⁺=279.1; HPLC Conditions A; ¹H NMR (400 MHz, D₂O) δ 4.34 (t, J=6.6 Hz, 1H), 3.85 (s, 3H), 3.81-3.69 (m, 2H), 3.66-3.51 (m, 2H), 2.57-2.37 (m, 2H), 2.20-2.01 (m, 3H), 1.85-1.71 (m, 2H), 1.53-1.35 (m, 2H), 0.95-0.86 (m, 3H).

### Exemplary Embodiment 1y

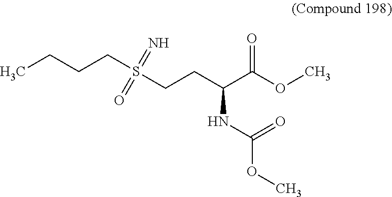 [SMILES: CCCCS(=N)(=O)CC[C@H](NC(=O)OC)C(=O)OC]

### (2S)-methyl 4-(butylsulfonimidoyl)-2-((methoxycarbonyl)amino)butanoate

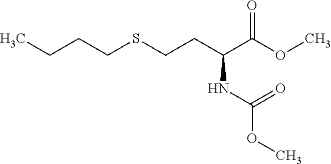 [SMILES: CCCCSCC[C@H](NC(=O)OC)C(=O)OC]

To a solution of methyl S-butyl-L-homocysteinate hydrochloride (150 mg, 620 mmol) in DCM (3 mL) was added TEA (314 mg, 3.10 mmol, 432 mL) and methyl carbonochloridate (122 mg, 1.29 mmol, 0.1 mL). The mixture was stirred at 20° C. for 3 h. The mixture was quenched by sat. NaHCO₃ (5 mL) and the aqueous phase was extracted with DCM (10 mL×2). The combined organic phase was dried with anhydrous Na₂SO₄, filtered and concentrated to give (S)-methyl 4-(butylthio)-2-((methoxycarbonyl)amino)butanoate (140 mg, 86% yield) as a yellow oil. ¹H NMR (400 MHz, CDCl₃-d) δ 5.37-5.25 (m, 1H), 4.56-4.41 (m, 1H), 3.78-3.75 (m, 3H), 2.53 (td, J=7.4, 14.7 Hz, 4H), 2.21-2.06 (m, 1H), 1.96 (br dd, J=7.2, 14.2 Hz, 1H), 1.61-1.50 (m, 5H), 1.47-1.34 (m, 2H), 0.92 (t, J=7.3 Hz, 3H).

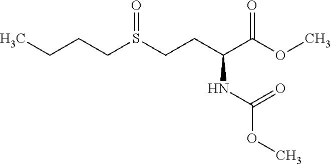 [SMILES: CCCCS(=O)CC[C@H](NC(=O)OC)C(=O)OC]

To a solution of (S)-methyl 4-(butylthio)-2-((methoxycarbonyl)amino)butanoate (110 mg, 418 mmol) in DCM (3 mL) was added m-CPBA (84.8 mg, 418 mmol, 85%). The mixture was stirred at 15° C. for 2 h. The reaction was quenched by NaHCO₃ (10 mL) and then extracted with DCM (10 mL×2). The combined organic phase was washed with Na₂SO₃ (5 mL), dried over anhydrous Na₂SO₄, filtered, and concentrated. The residue was purified by prep-TLC (SiO₂, Ethyl acetate:Methanol=0:1, R_{f}=0.45) to give (2S)-methyl 4-(butylsulfinyl)-2-((methoxycarbonyl)amino)butanoate (100 mg, 86% yield) as a yellow oil.

¹H NMR (400 MHz, CDCl₃-d) δ 5.78-5.41 (m, 1H), 4.49 (br s, 1H), 3.79 (s, 3H), 3.70 (s, 3H), 2.84-2.60 (m, 4H), 2.41 (br d, J=6.4 Hz, 1H), 2.27-2.11 (m, 1H), 1.80-1.71 (m, 2H), 1.59-1.42 (m, 2H), 0.98 (t, J=7.3 Hz, 3H).

To a solution of (2S)-methyl 4-(butylsulfinyl)-2-((methoxycarbonyl)amino)butanoate (80.0 mg, 286 mmol) in MeOH (2 mL) was added ammonium carbamate (112 mg, 1.43 mmol) and PhI(OAc)₂ (277 mg, 859 mmol). The mixture was stirred at 15° C. for 16 h. Additional ammonium carbamate (22.4 mg, 286 umol) and PhI(OAc)₂ (92.2 mg, 286 umol) was added and the mixture was stirred at 15° C. for 2 h. The mixture was poured into water (10 mL) and extracted with DCM (15 mL×3). The residue was purified by prep-TLC (SiO₂, Petroleum ether: ethyl acetate=0:1, R_{f}=0.4) to give (2S)-methyl 4-(butylsulfonimidoyl)-2-((methoxycarbonyl)amino)butanoate (Compound 198) (25 mg, 30% yield) as colorless oil. LCMS: Rt=2.240 min., (ES⁺) m/z (M+H)⁺=295.1; HPLC Conditions: A; ¹H NMR (400 MHz, D₂O) δ 4.47-4.32 (m, 1H), 3.76 (s, 3H), 3.66 (s, 3H), 3.38-3.09 (m, 4H), 2.44-2.30 (m, 1H), 2.20-2.07 (m, 1H), 1.80-1.66 (m, 2H), 1.52-1.37 (m, 2H), 0.91 (t, J=7.4 Hz, 3H).

### Exemplary Embodiment 1z

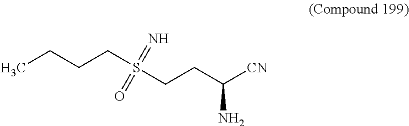

### (2S)-2-amino-4-(butylsulfonimidoyl)butanenitrile

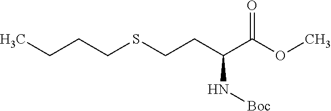 [SMILES: CCCCSCC[C@H](NC(=O)OC(C)(C)C)C(=O)OC]

To a solution of (S)-methyl 2-((tert-butoxycarbonyl)amino)-4-mercaptobutanoate (1.0 g, 4.01 mmol) and 1-bromobutane (824 mg, 6.02 mmol, 649 mL) in DMF (10 mL) was added K₂CO₃ (1.11 g, 8.02 mmol). The mixture was stirred at 20° C. for 16 h. The solution was adjusted to pH˜7 with citric acid and extracted with MTBE (20 mL×2). The combined organic extracts were dried over Na₂SO₄, filtered, and concentrated. The crude product was used directly without purification. (S)-methyl 2-((tert-butoxycarbonyl)amino)-4-(butylthio)butanoate (1.1 g, 90% yield) was obtained as a colorless oil. ¹H NMR (400 MHz, CDCl₃-d) δ 5.11 (br d, J=5.4 Hz, 1H), 4.42 (br d, J=4.3 Hz, 1H), 3.76 (s, 3H), 2.58-2.48 (m, 4H), 2.18-2.07 (m, 1H), 1.98-1.83 (m, 1H), 1.60-1.54 (m, 2H), 1.47-1.39 (m, 11H), 0.92 (t, J=7.3 Hz, 3H).

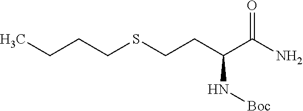 [SMILES: CCCCSCC[C@H](NC(=O)OC(C)(C)C)C(N)=O]

To a solution of (S)-methyl 2-((tert-butoxycarbonyl)amino)-4-(butylthio)butanoate (0.5 g, 1.64 mmol) in NH₃/MeOH (7 M, 30 mL) was stirred at 30° C. for 48 h in a sealed tube. The reaction mixture was concentrated to give (S)-tert-butyl (1-amino-4-(butylthio)-1-oxobutan-2-yl)carbamate (0.47 g, 99% yield) as a white solid. ¹H NMR (400 MHz, CDCl₃-d) δ 6.25 (br s, 1H), 5.60 (br s, 1H), 5.23 (br d, J=8.1 Hz, 1H), 4.31 (br d, J=5.0 Hz, 1H), 2.69-2.48 (m, 4H), 2.15-2.03 (m, 1H), 1.93 (qd, J=7.3, 14.3 Hz, 1H), 1.57 (quin, J=7.4 Hz, 2H), 1.45 (s, 9H), 1.43-1.35 (m, 2H), 0.92 (t, J=7.3 Hz, 3H).

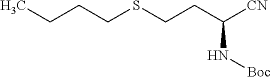 [SMILES: CCCCSCC[C@@H](C#N)NC(=O)OC(C)(C)C]

To a solution of (S)-tert-butyl (1-amino-4-(butylthio)-1-oxobutan-2-yl)carbamate (0.1 g, 344 mmol) in THE (3 mL) was added pyridine (136 mg, 1.72 mmol, 139 mL) and trifluoroacetic anhydride (217 mg, 1.03 mmol, 144 mL) in THE (0.5 mL) at 0° C. The mixture was stirred at 0° C. for 1 h then at 20° C. for 4 h. The mixture was poured into water (10 mL) and extracted with DCM (10 mL×2). The combined organic extracts were dried over Na₂SO₄, filtered, and concentrated. The residue was purified by prep-TLC (SiO₂, Petroleum ether: ethyl acetate=3:1, R_{f}=0.6) to give (S)-tert-butyl (3-(butylthio)-1-cyanopropyl)carbamate (80 mg, 85% yield) as a yellow oil. ¹H NMR (400 MHz, CDCl₃-d) δ 5.12-4.65 (m, 2H), 2.72-2.66 (m, 2H), 2.58-2.51 (m, 2H), 2.09 (q, J=7.2 Hz, 2H), 1.63-1.56 (m, 2H), 1.51-1.37 (m, 11H), 0.93 (t, J=7.3 Hz, 3H).

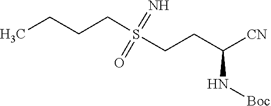 [SMILES: CCCCS(=N)(=O)CC[C@@H](C#N)NC(=O)OC(C)(C)C]

To a solution of (S)-tert-butyl (3-(butylthio)-1-cyanopropyl)carbamate (80 mg, 294 mmol) in MeOH (3 mL) was added PhI(OAc)₂ (284 mg, 881 mmol) and ammonium carbamate (138 mg, 1.76 mmol). The mixture was stirred at 20° C. for 4 h and concentrated. H₂O (10 mL) was added and extracted with ethyl acetate (15 mL×2). The combined organic extracts were dried over Na₂SO₄, filtered, and concentrated. The residue was purified by prep-TLC (SiO₂, Petroleum ether: ethyl acetate=0:1, R_{f}=0.4) to give tert-butyl ((1S)-3-(butylsulfonimidoyl)-1-cyanopropyl)carbamate (60 mg, 66% yield) as a yellow oil. LCMS: Rt=2.059 min., (ES⁺) m/z (M+H)⁺=304.1; ¹H NMR (400 MHz, CDCl₃-d) δ 6.03 (br d, J=7.6 Hz, 1H), 5.55 (br d, J=8.4 Hz, 1H), 4.80 (br s, 1H), 3.35-3.00 (m, 4H), 2.52-2.38 (m, 2H), 1.91-1.80 (m, 2H), 1.54-1.44 (m, 11H), 0.99 (t, J=7.3 Hz, 3H).

To a solution of tert-butyl ((1S)-3-(butylsulfonimidoyl)-1-cyanopropyl)carbamate (25 mg, 82.4 mmol) in HCl/dioxane (4 M, 5 mL) was stirred at 15° C. for 2 h. The reaction mixture was concentrated to give (2S)-2-amino-4-(butylsulfonimidoyl)butanenitrile (Compound 199) (21.1 mg, 96% yield, HCl) as a yellow solid. LCMS: Rt=0.282 min., (ES⁺) m/z (M+H)⁺=204.1; HPLC Conditions: A; ¹H NMR (400 MHz, D₂O) δ 3.76-3.26 (m, 5H), 2.63-2.36 (m, 2H), 1.92-1.71 (m, 2H), 1.55-1.36 (m, 2H), 0.97-0.85 (m, 3H).

### Exemplary Embodiment 1aa1

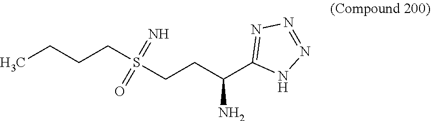 [SMILES: CCCCS(=N)(=O)CC[C@H](N)c1nnn[nH]1]

### (1S)-3-(butylsulfonimidoyl)-1-(1H-tetrazol-5-yl)propan-1-amine

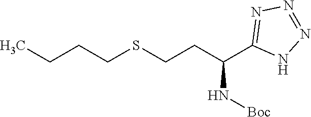 [SMILES: CCCCSCC[C@H](NC(=O)OC(C)(C)C)c1nnn[nH]1]

(S)-tert-butyl (3-(butylthio)-1-(1H-tetrazol-5-yl)propyl)carbamate: To a solution of (S)-tert-butyl (3-(butylthio)-1-cyanopropyl)carbamate (180 mg, 661 mmol) in i-PrOH (3 mL) and H₂O (1.8 mL) was added NaN₃ (107 mg, 1.65 mmol) and ZnBr₂ (119 mg, 529 mmol, 26.5 μL). The mixture was stirred at 100° C. for 16 h. The reaction mixture was cooled and adjusted to pH˜7 with citric acid and extracted with ethyl acetate (20 mL×3). The combined organic extracts were dried over Na₂SO₄, filtered, and concentrated. The residue was purified by prep-TLC (SiO₂, Petroleum ether: ethyl acetate=0:1, R_{f}=0.2) to give (S)-tert-butyl (3-(butylthio)-1-(1H-tetrazol-5-yl)propyl)carbamate (130 mg, 62% yield) as a yellow oil. ¹H NMR (400 MHz, CDCl₃-d) δ 5.82-5.42 (m, 1H), 5.29-4.99 (m, 1H), 2.74-2.21 (m, 6H), 1.59-1.48 (m, 2H), 1.45-1.28 (m, 11H), 0.89 (br d, J=3.7 Hz, 3H).

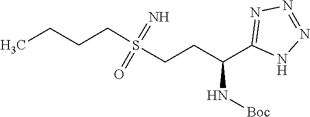 [SMILES: CCCCS(=N)(=O)CC[C@H](NC(=O)OC(C)(C)C)c1nnn[nH]1]

To a solution of (S)-tert-butyl (3-(butylthio)-1-(1H-tetrazol-5-yl)propyl)carbamate (70.0 mg, 222 μmol) in MeOH (2 mL) was added PhI(OAc)₂ (286 mg, 888 μmol) and ammonium carbamate (139 mg, 1.78 mmol). The mixture was stirred at 20° C. for 6 h. PhI(OAc)₂ (286 mg, 888 μmol) and ammonium carbamate (139 mg, 1.78 mmol) was added. The mixture was stirred at 20° C. for 16 h. The reaction mixture was concentrated and water (10 mL) was added and extracted with ethyl acetate (15 mL×2). The aqueous phase was concentrated and the residue was purified by prep-HPLC (column: Phenomenex Gemini-NX C18 75×30 mm, 3 microns; mobile phase: [water (0.2% formic acid)-MeCN]; B %: 5%-35%, 12 min) to give tert-butyl ((1S)-3-(butylsulfonimidoyl)-1-(1H-tetrazol-5-yl)propyl)carbamate (35 mg, 40% yield, formic acid) as a white solid. ¹H NMR (400 MHz, CDCl₃-d) δ 8.08-7.47 (m, 2H), 6.48 (br d, J=5.4 Hz, 1H), 6.16-5.98 (m, 1H), 5.49-5.18 (m, 1H), 3.49-3.06 (m, 4H), 2.77-2.43 (m, 2H), 1.90-1.78 (m, 2H), 1.55-1.38 (m, 11H), 0.98 (t, J=7.3 Hz, 3H).

To a solution of tert-butyl ((1S)-3-(butylsulfonimidoyl)-1-(1H-tetrazol-5-yl)propyl)carbamate (25.0 mg, 72.2 μmol) in HCl/dioxane (4 M, 2 mL) was stirred at 15° C. for 1 h. The reaction mixture was concentrated to give (1S)-3-(butylsulfonimidoyl)-1-(1H-tetrazol-5-yl)propan-1-amine (Compound 200) (18.7 mg, 84% yield, HCl) as a yellow solid. LCMS: Rt=0.638 min., (ES⁺) m/z (M+H)⁺=247.1; HPLC Conditions: A; ¹H NMR (400 MHz, D₂O) δ 5.07 (br t, J=6.8 Hz, 1H), 3.77-3.66 (m, 2H), 3.60 (br d, J=6.5 Hz, 2H), 2.79-2.70 (m, 2H), 1.86-1.69 (m, 2H), 1.53-1.35 (m, 2H), 0.97-0.85 (m, 3H).

### Exemplary Embodiment 1aa2

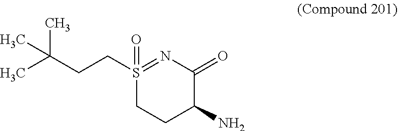

### (1S,4R)-4-amino-1-(3,3-dimethylbutyl)-5,6-dihydro-1,2-thiazin-3 (4H)-one 1-oxide

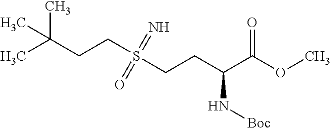 [SMILES: COC(=O)[C@H](CCS(=N)(=O)CCC(C)(C)C)NC(=O)OC(C)(C)C]

To a solution of methyl N-(tert-butoxycarbonyl)-S-(3,3-dimethylbutyl)-L-homocysteinate (3 g, 9 mmol) in MeOH (24 mL) was added PhI(OAc)₂ (11.9 g, 36 mmol) and ammonium carbamate (5.62 g, 71.9 mmol). The mixture was stirred at 20° C. for 16 h. The reaction mixture was concentrated, added water (30 mL) and extracted with EtOAc (45 mL×3). The combined organic extracts were dried over Na₂SO₄, filtered, and the filtrate was concentrated. The residue was purified by column chromatography (SiO₂, Petroleum ether: ethyl acetate=1:1) to give (2S)-methyl 2-((tert-butoxycarbonyl)amino)-4-(3,3-dimethylbutylsulfonimidoyl)butanoate (1.60 g, 49% yield), as a colorless oil. ¹H NMR (400 MHz, CDCl₃-d) δ 5.39-5.23 (m, 1H), 4.44 (br s, 1H), 3.79 (s, 3H), 3.23-2.94 (m, 4H), 2.50-2.36 (m, 1H), 2.24-2.10 (m, 1H), 1.78-1.67 (m, 2H), 1.46 (s, 9H), 0.96 (s, 9H).

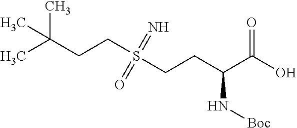 [SMILES: CC(C)(C)CCS(=N)(=O)CC[C@H](NC(=O)OC(C)(C)C)C(=O)O]

To a solution of (2S)-methyl 2-((tert-butoxycarbonyl)amino)-4-(3,3-dimethylbutylsulfonimidoyl)butanoate (1.60 g, 4.39 mmol) in THE (12 mL) and H₂O (4 mL) was added LiOH·H₂O (368 mg, 8.78 mmol). The mixture was stirred at 25° C. for 2 h. The solution was adjusted to pH˜5 with citric acid and extracted with EtOAc (20 mL×4). The combined organic extracts were dried over Na₂SO₄, filtered, and concentrated to give (2S)-2-((tert-butoxycarbonyl)amino)-4-(3,3-dimethylbutylsulfonimidoyl)butanoic acid (1.30 g, 85% yield) as a white solid. ¹H NMR (400 MHz, CDCl₃-d) δ 5.69 (br d, J=6.5 Hz, 1H), 4.55-4.37 (m, 1H), 3.53-3.17 (m, 4H), 2.47-2.21 (m, 2H), 1.82-1.65 (m, 2H), 1.44 (s, 9H), 0.96 (d, J=3.4 Hz, 9H).

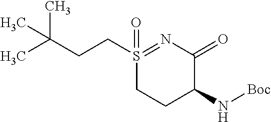 [SMILES: CC(C)(C)CCS1(=O)=NC(=O)[C@@H](NC(=O)OC(C)(C)C)CC1]

To a solution of (2S)-2-((tert-butoxycarbonyl)amino)-4-(3,3-dimethylbutylsulfonimidoyl)butanoic acid (200 mg, 570 μmol) in DMF (2 mL) was added HATU (325 mg, 855 μmol) and DIEA (295 mg, 2.28 mmol). The mixture was stirred at 25° C. for 16 h. The reaction mixture was poured into water (5 mL) and extracted with EtOAc (5 mL×3). The combined organic extracts were washed with brine (5 mL), dried over Na₂SO₄, filtered, and concentrated to give a residue. The residue was purified by prep-TLC (SiO₂, Petroleum ether: ethyl acetate=1:1) to give tert-butyl ((1S,4R)-1-(3,3-dimethylbutyl)-1-oxido-3-oxo-3,4,5,6-tetrahydro-1,2-thiazin-4-yl)carbamate (55.0 mg, 29% yield) as a colorless oil. ¹H NMR (400 MHz, CDCl₃-d) δ 5.69 (br s, 1H), 4.31-3.97 (m, 1H), 3.56-3.41 (m, 2H), 3.29-3.19 (m, 2H), 2.89 (br dd, J=4.0, 8.6 Hz, 1H), 2.38 (br d, J=9.3 Hz, 1H), 1.80-1.71 (m, 2H), 1.44 (s, 9H), 0.97 (s, 9H).

A mixture of tert-butyl ((1S,4R)-1-(3,3-dimethylbutyl)-1-oxido-3-oxo-3,4,5,6-tetrahydro-1,2-thiazin-4-yl)carbamate (55.0 mg, 165 μmol) in TFA (0.3 mL) and DCM (0.9 mL) was stirred at 25° C. for 2 h. The reaction mixture was concentrated. The residue was poured into water (1 mL) and extracted with DCM (1 mL×2). The aqueous phase was lyophilized to give (1S,4R)-4-amino-1-(3,3-dimethylbutyl)-5,6-dihydro-1,2-thiazin-3 (4H)-one 1-oxide (Compound 201) (34.0 mg, 86% yield) as a white solid. LCMS: Rt=1.929 min., (ES⁺) m/z (M+H)⁺=233.1.; HPLC Conditions: B; ¹H NMR (400 MHz, D₂O) δ 4.31-4.10 (m, 1H), 4.04-3.46 (m, 4H), 2.80-2.37 (m, 2H), 1.83-1.65 (m, 2H), 0.99-0.89 (m, 9H).

### Exemplary Embodiment 1aa3

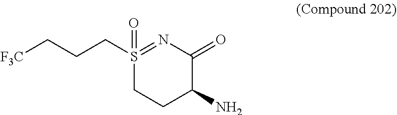 [SMILES: N[C@H]1CCS(=O)(CCCC(F)(F)F)=NC1=O]

### (1S,4R)-4-amino-1-(4,4,4-trifluorobutyl)-5,6-dihydro-1,2-thiazin-3 (4H)-one 1-oxide

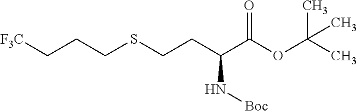 [SMILES: CC(C)(C)OC(=O)N[C@@H](CCSCCCC(F)(F)F)C(=O)OC(C)(C)C]

A mixture of (S)-tert-butyl 2-((tert-butoxycarbonyl)amino)-4-mercaptobutanoate (400 mg, 1.37 mmol), 1,1,1-trifluoro-4-iodobutane (327 mg, 1.37 mmol) and K₂CO₃ (379 mg, 2.75 mmol) in DMF (6 mL) was degassed and purged with N₂ for 3 times, and then the mixture was stirred at 25° C. for 16 h under N₂ atmosphere. The reaction mixture was filtered, and the filtrate was concentrated. The residue was partitioned between EtOAc (20 mL) and water (5 mL) and the organic phase was separated and concentrated to give a residue. The residue was purified by column chromatography (SiO₂, Petroleum ether: ethyl acetate=1:0 to 97:3) to give (S)-tert-butyl 2-((tert-butoxycarbonyl)amino)-4-((4,4,4-trifluorobutyl) thio)butanoate (420 mg, 76% yield) as a yellow oil. ¹H NMR (400 MHz, CDCl₃-d) δ 5.03 (br d, J=7.2 Hz, 1H), 4.21 (br d, J=5.0 Hz, 1H), 2.59-2.38 (m, 4H), 2.24-2.07 (m, 2H), 2.01 (br d, J=6.6 Hz, 1H), 1.87-1.67 (m, 3H), 1.46-1.29 (m, 18H).

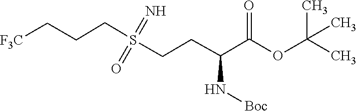 [SMILES: CC(C)(C)OC(=O)N[C@@H](CCS(=N)(=O)CCCC(F)(F)F)C(=O)OC(C)(C)C]

To a solution of compound (S)-tert-butyl 2-((tert-butoxycarbonyl)amino)-4-((4,4,4-trifluorobutyl) thio)butanoate (410 mg, 1.02 mmol) in i-PrOH (10 mL) was added PhI(OAc)₂ (987 mg, 3.06 mmol) and ammonium carbamate (399 mg, 5.11 mmol). The mixture was stirred at 25° C. for 16 h. The reaction mixture was concentrated to remove i-PrOH. The residue was diluted with DCM (30 mL), washed with water (8 mL) and the organic phase was concentrated. The residue was purified by column chromatography (SiO₂, Petroleum ether: ethyl acetate=20:1 to 3:1) to give (2S)-tert-butyl 2-((tert-butoxycarbonyl)amino)-4-(4,4,4-trifluorobutylsulfonimidoyl)butanoate (370 mg, 88% yield) as a colorless oil. ¹H NMR (400 MHz, CDCl₃-d) δ 5.23 (br s, 1H), 4.35-4.19 (m, 1H), 3.28-2.97 (m, 4H), 2.51-2.23 (m, 4H), 2.22-2.05 (m, 4H), 1.46 (d, J=15.1 Hz, 18H).

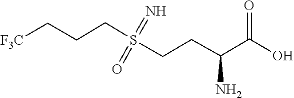 [SMILES: N=S(=O)(CCCC(F)(F)F)CC[C@H](N)C(=O)O]

To a solution of (2S)-tert-butyl 2-((tert-butoxycarbonyl)amino)-4-(4,4,4-trifluorobutylsulfonimidoyl)butanoate (70.0 mg, 162 μmol) in DCM (1.5 mL) was added TFA (616 mg, 5.40 mmol, 0.4 mL). The mixture was stirred at 25° C. for 16 h. The reaction mixture was concentrated to give (2S)-2-amino-4-(4,4,4-trifluorobutylsulfonimidoyl)butanoic acid (54 mg, 81% yield, TFA) as a yellow oil. LCMS: Rt=0.607 min, (ES⁺) m/z (M+H)⁺=277.0; ¹H NMR (400 MHz, D₂O) δ 4.13 (br t, J=6.5 Hz, 1H), 4.04-3.60 (m, 4H), 2.56-2.27 (m, 4H), 2.14 (quin, J=7.8 Hz, 2H). (2S)-2-amino-4-(4,4,4-trifluorobutylsulfonimidoyl)butanoic acid was converted slowly over time to a byproduct that was isolated. Briefly, the mixture (30.0 mg, 108 μmol) was dissolved in water/MeOH (2:1, 1.5 mL) and purified by prep-HPLC (column: Phenomenex C18 75×30 mm, 3 microns; mobile phase: [water(NH₄HCO₃)-MeCN]; B %: 5%-35%, 12 min) to give (1S,4R)-4-amino-1-(4,4,4-trifluorobutyl)-5,6-dihydro-1,2-thiazin-3 (4H)-one 1-oxide (4.9 mg, 17% yield) as a yellow oil. LCMS: Rt=0.697 min, (ES⁺) m/z (M+H)⁺=259.0. HPLC Conditions: E; ¹H NMR (400 MHz, MeOD) δ 3.84-3.64 (m, 1H), 3.62-3.39 (m, 3H), 2.56-2.28 (m, 3H), 2.27-2.04 (m, 2H).

### Exemplary Embodiment 1aa4

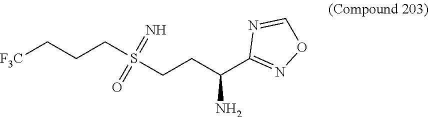 [SMILES: N=S(=O)(CCCC(F)(F)F)CC[C@H](N)c1ncon1]

### (1S)-1-(1,2,4-oxadiazol-3-yl)-3-(4,4,4-trifluorobutylsulfonimidoyl)propan-1-amine

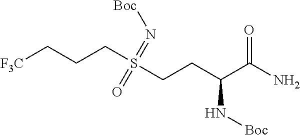 [SMILES: CC(C)(C)OC(=O)N=S(=O)(CCCC(F)(F)F)CC[C@H](NC(=O)OC(C)(C)C)C(N)=O]

A mixture of (2S)-4-(N-(tert-butoxycarbonyl)-4,4,4-trifluorobutylsulfonimidoyl)-2-((tert-butoxycarbonyl)amino)butanoic acid (1.50 g, 3.15 mmol), NH₄Cl (505 mg, 9.44 mmol), HATU (1.44 g, 3.78 mmol) and DIEA (2.03 g, 15.7 mmol, 2.74 mL) in DMF (30 mL) was stirred at 20° C. for 12 h. The mixture was quenched with water (50 mL) and extracted with ethyl acetate (20 mL×2). The combined organic extracts were washed with brine (30 mL), dried over Na₂SO₄, filtered, and concentrated. The residue was purified by silica gel chromatography (Petroleum ether: ethyl acetate=1:1) to give tert-butyl (((S)-4-amino-3-((tert-butoxycarbonyl)amino)-4-oxobutyl)(oxo)(4,4,4-trifluorobutyl)-l⁶-sulfaneylidene)carbamate (1.2 g, 80% yield) as a white solid. ¹H NMR (400 MHz, CDCl₃-d) δ 6.73-6.39 (m, 1H), 5.64-5.40 (m, 2H), 4.37 (br d, J=5.9 Hz, 1H), 3.67-3.32 (m, 4H), 2.47-2.08 (m, 6H), 1.47 (d, J=13.2 Hz, 18H).

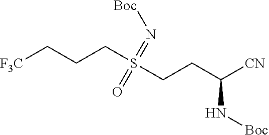 [SMILES: CC(C)(C)OC(=O)N=S(=O)(CCCC(F)(F)F)CC[C@@H](C#N)NC(=O)OC(C)(C)C]

To a solution of tert-butyl (((S)-4-amino-3-((tert-butoxycarbonyl)amino)-4-oxobutyl)(oxo)(4,4,4-trifluorobutyl)-l⁶-sulfaneylidene)carbamate (600 mg, 1.26 mmol) and TEA (511 mg, 5.05 mmol, 702 μL) in DCM (10 mL) was added TFAA (530 mg, 2.52 mmol, 351 μL) dropwise at 0° C. The mixture was stirred at 20° C. for 4 h. The mixture was diluted with DCM (10 mL), washed with 0.5M HCl (2 mL), sat. NaHCO₃ (3 mL), and the organic phase was dried over Na₂SO₄, filtered and concentrated to give tert-butyl (((S)-3-((tert-butoxycarbonyl)amino)-3-cyanopropyl)(oxo)(4,4,4-trifluorobutyl)-l⁶-sulfaneylidene)carbamate (0.59 g, crude) as a colorless oil. ¹H NMR (400 MHz, CDCl₃-d) δ 5.40-5.19 (m, 1H), 4.81-4.64 (m, 1H), 3.64-3.48 (m, 2H), 3.46-3.31 (m, 2H), 2.54-2.41 (m, 2H), 2.40-2.24 (m, 2H), 2.23-2.12 (m, 2H), 1.51-1.44 (m, 18H).

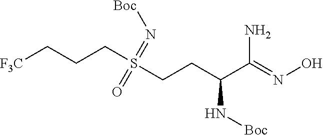 [SMILES: CC(C)(C)OC(=O)N=S(=O)(CCCC(F)(F)F)CC[C@H](NC(=O)OC(C)(C)C)/C(N)=N/O]

A mixture of tert-butyl (((S)-3-((tert-butoxycarbonyl)amino)-3-cyanopropyl)(oxo)(4,4,4-trifluorobutyl)-16-sulfaneylidene)carbamate (100 mg, 219 mmol), NH₂OH·HCl (22.8 mg, 328 mmol) and TEA (66.4 mg, 656 mmol, 91.3 μL) in ethanol (3 mL) was heated at 80° C. for 2 h. The mixture was concentrated, the residue was added water (10 mL) and extracted with ethyl acetate (10 mL×2), the combined organic phases were washed with brine (10 mL), dried over Na₂SO₄, filtered, and concentrated to give tert-butyl (((S,Z)-4-amino-3-((tert-butoxycarbonyl)amino)-4-(hydroxyimino)butyl)(oxo)(4,4,4-trifluorobutyl)-16-sulfaneylidene)carbamate (110 mg, crude) as yellow oil. ¹H NMR (400 MHz, CDCl₃-d) δ 6.42-5.90 (m, 1H), 5.28-5.15 (m, 1H), 4.99-4.78 (m, 2H), 4.36-4.24 (m, 1H), 3.64-3.28 (m, 4H), 2.48-2.09 (m, 6H), 1.47 (d, J=13.8 Hz, 18H).

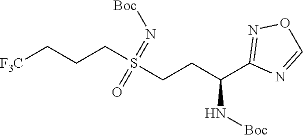 [SMILES: CC(C)(C)OC(=O)N=S(=O)(CCCC(F)(F)F)CC[C@H](NC(=O)OC(C)(C)C)c1ncon1]

A mixture of tert-butyl (((S,Z)-4-amino-3-((tert-butoxycarbonyl)amino)-4-(hydroxyimino)butyl)(oxo)(4,4,4-trifluorobutyl)-16-sulfaneylidene)carbamate (110 mg, 224 μmol) and PPTS (2.82 mg, 11.2 μmol) in trimethoxymethane (2 mL) was heated at 70° C. for 4 h. The mixture was concentrated, the residue was diluted with ethyl acetate (10 mL), washed with water (10 mL), dried over Na₂SO₄, filtered and concentrated. The residue was purified by prep-TLC (Petroleum ether: ethyl acetate=1:1) to give tert-butyl ((1S)-3-(N-(tert-butoxycarbonyl)-4,4,4-trifluorobutylsulfonimidoyl)-1-(1,2,4-oxadiazol-3-yl)propyl)carbamate (80 mg, 71% yield) as colorless oil. ¹H NMR (400 MHz, CDCl₃-d) δ 8.73 (s, 1H), 5.36-5.22 (m, 1H), 5.20-5.01 (m, 1H), 3.60-3.27 (m, 4H), 2.57-2.23 (m, 4H), 2.10 (s, 2H), 1.47 (d, J=9.0 Hz, 18H).

A solution of tert-butyl ((1S)-3-(N-(tert-butoxycarbonyl)-4,4,4-trifluorobutylsulfonimidoyl)-1-(1,2,4-oxadiazol-3-yl)propyl)carbamate (70 mg, 140 μmol) in HCl/dioxane (2 mL) was stirred at 20° C. for 2 h. The mixture was concentrated and the residue was added water (20 mL) and lyophilized to give (1S)-1-(1,2,4-oxadiazol-3-yl)-3-(4,4,4-trifluorobutylsulfonimidoyl)propan-1-amine (Compound 203) (50 mg, 2HCl) as yellow solid. LCMS: Rt=0.998 min., (ES⁺) m/z (M+H)⁺=301.0; HPLC Conditions: E; ¹H NMR (400 MHz, D₂O) δ 9.28 (s, 1H), 5.00-4.89 (m, 1H), 3.63-3.36 (m, 4H), 2.67-2.54 (m, 2H), 2.44-2.23 (m, 2H), 2.04 (quin, J=7.7 Hz, 2H).

### Exemplary Embodiment 1aa5

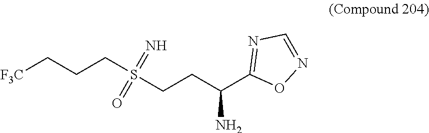 [SMILES: N=S(=O)(CCCC(F)(F)F)CC[C@H](N)c1ncno1]

### (1S)-1-(1,2,4-oxadiazol-5-yl)-3-(4,4,4-trifluorobutylsulfonimidoyl)propan-1-amine

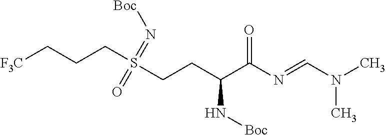 [SMILES: CN(C)/C=N/C(=O)[C@H](CCS(=O)(CCCC(F)(F)F)=NC(=O)OC(C)(C)C)NC(=O)OC(C)(C)C]

A mixture of (((S)-4-amino-3-((tert-butoxycarbonyl)amino)-4-oxobutyl)(oxo)(4,4,4-trifluorobutyl)-l⁶-sulfaneylidene)carbamate (300 mg, 631 μmol) and DMFDMA (150 mg, 1.26 mmol) in toluene (3 mL) and DMF (1 mL) was heated at 100° C. for 2 h. The mixture was concentrated, the residue was dissolved in ethyl acetate (10 mL), washed with water (5 mL×2), brine (5 mL), the organic phase was dried over Na₂SO₄, filtered, and concentrated to give tert-butyl (((S)-3-((tert-butoxycarbonyl)amino)-4-(((E)-(dimethylamino)methylene)amino)-4-oxobutyl)(oxo)(4,4,4-trifluorobutyl)-16-sulfaneylidene)carbamate (0.33 g, crude), as a yellow oil. ¹H NMR (400 MHz, CDCl₃-d) δ 8.47 (s, 1H), 5.79-5.54 (m, 1H), 4.49-4.23 (m, 1H), 3.57-3.30 (m, 4H), 3.26-3.08 (m, 6H), 2.56-2.42 (m, 1H), 2.37-2.22 (m, 3H), 2.19-2.07 (m, 2H), 1.49-1.43 (m, 18H).

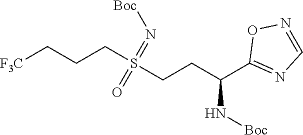 [SMILES: CC(C)(C)OC(=O)N=S(=O)(CCCC(F)(F)F)CC[C@H](NC(=O)OC(C)(C)C)c1ncno1]

A mixture of tert-butyl (((S)-3-((tert-butoxycarbonyl)amino)-4-(((E)-(dimethylamino)methylene)amino)-4-oxobutyl)(oxo)(4,4,4-trifluorobutyl)-l⁶-sulfaneylidene)carbamate (200 mg, 377 μmol) and NH₂OH·HCl (52.4 mg, 754 μmol) in EtOH (3 mL) was heated at 80° C. for 16 h. The mixture was concentrated and the residue was diluted with water (10 mL) and extracted with ethyl acetate (10 mL×2). The combined organic phases were dried over Na₂SO₄, filtered and concentrated to give tert-butyl ((1S)-3-(N-(tert-butoxycarbonyl)-4,4,4-trifluorobutylsulfonimidoyl)-1-(1,2,4-oxadiazol-5-yl)propyl)carbamate (0.2 g, crude) as yellow oil.

To a solution of tert-butyl ((1S)-3-(N-(tert-butoxycarbonyl)-4,4,4-trifluorobutylsulfonimidoyl)-1-(1,2,4-oxadiazol-5-yl)propyl)carbamate (0.2 g, 400 μmol) in DCM (3 mL) was added TFA (1 mL), the mixture was stirred at 20° C. for 1 h. The mixture was concentrated and the residue was purified by prep-HPLC (column: C18-1 150×mm, 5 microns; mobile phase: [water(formic acid)-MeCN]; B %: 1%-30%, 10 min) to give (1S)-1-(1,2,4-oxadiazol-5-yl)-3-(4,4,4-trifluorobutylsulfonimidoyl)propan-1-amine (Compound 204) (40 mg, 27% yield, formic acid salt) as yellow oil. LCMS: Rt=1.514 min., (ES⁺) m/z (M+H)⁺=301.0; HPLC Conditions: A; ¹H NMR (400 MHz, D₂O) δ 8.81 (s, 1H), 5.17 (dt, J=3.9, 6.9 Hz, 1H), 3.57-3.45 (m, 2H), 3.45-3.34 (m, 2H), 2.76-2.61 (m, 2H), 2.45-2.30 (m, 2H), 2.14-2.00 (m, 2H).

### Exemplary Embodiment 1aa6

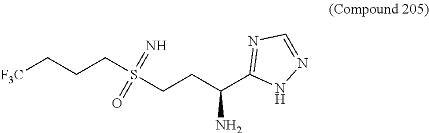 [SMILES: N=S(=O)(CCCC(F)(F)F)CC[C@H](N)c1ncn[nH]1]

### (1S)-1-(1H-1,2,4-triazol-5-yl)-3-(4,4,4-trifluorobutylsulfonimidoyl)propan-1-amine

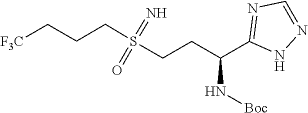 [SMILES: CC(C)(C)OC(=O)N[C@@H](CCS(=N)(=O)CCCC(F)(F)F)c1ncn[nH]1]

A solution of give tert-butyl (((S)-3-((tert-butoxycarbonyl)amino)-4-(((E)-(dimethylamino)methylene)amino)-4-oxobutyl)(oxo)(4,4,4-trifluorobutyl)-l⁶-sulfaneylidene)carbamate (130 mg, 245 μmol) in acetic acid (3 mL) was added N₂H₄·H₂O (18.4 mg, 367 μmol, 17.8 μL). The mixture was stirred at 90° C. for 2 h. The mixture was concentrated, and the residue was diluted with water (20 mL) and extracted with dichloromethane (20 mL). The organic layer was dried over sodium sulfate, filtered, and concentrated. The crude product (100 mg) was used for the next step directly.

A solution of tert-butyl ((1S)-1-(1H-1,2,4-triazol-5-yl)-3-(4,4,4-trifluorobutylsulfonimidoyl)propyl)carbamate (100 mg, 250 μmol) in dioxane (1 mL) was added HCl/dioxane (4 M, 3 mL). The mixture was stirred at 20° C. for 16 h then concentrated. The reaction was purified by prep-HPLC (column: C18-1 150×30 mm, 5 microns; mobile phase: [water(formic acid)-MeCN]; B %: 1%-25%, 10 min) to give (1S)-1-(1H-1,2,4-triazol-5-yl)-3-(4,4,4-trifluorobutylsulfonimidoyl)propan-1-amine (Compound 205) (28 mg, 35% yield) as white solid. LCMS: Rt=0.880 min., (ES⁺) m/z (M+H)⁺=300.1; HPLC Conditions: A; ¹H NMR (400 MHz, D₂O) δ 8.54 (s, 1H), 4.83-4.78 (m, 1H), 3.58-3.34 (m, 4H), 2.62 (q, J=7.5 Hz, 2H), 2.37 (br dd, J=9.2, 18.1 Hz, 2H), 2.11-2.01 (m, 2H).

### Exemplary Embodiment 1aa7

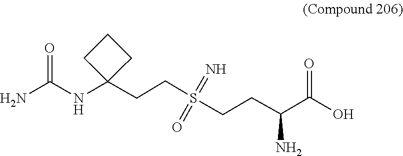 [SMILES: N=S(=O)(CC[C@H](N)C(=O)O)CCC1(NC(N)=O)CCC1]

### (2S)-2-amino-4-(2-(1-ureidocyclobutyl)ethylsulfonimidoyl)butanoic acid

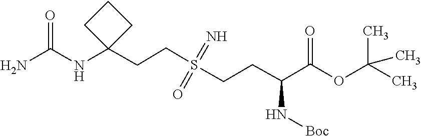 [SMILES: CC(C)(C)OC(=O)N[C@@H](CCS(=N)(=O)CCC1(NC(N)=O)CCC1)C(=O)OC(C)(C)C]

To a solution of (S)-tert-butyl 2-((tert-butoxycarbonyl)amino)-4-((2-(1-carbamoylcyclobutyl)ethyl)thio)butanoate (100 mg, 240 μmol) in i-PrOH (3 mL) was added PhI(OAc)₂ (193 mg, 600 μmol) ammonium carbamate (93.7 mg, 1.20 mmol) and the mixture was stirred at 15° C. for 16 h. The mixture was quenched with H₂O (30 mL) and extracted with ethyl acetate (30 mL×2). The combined organic extracts were washed with brine (20 mL×2), dried with anhydrous Na₂SO₄, filtered, and concentrated. The residue was purified by prep-HPLC: column: C18-1 150×30 mm, 5 microns; mobile phase: [water(NH₄HCO₃)-MeCN]; B %: 30%-60%, 10 min to give (2S)-tert-butyl 2-((tert-butoxycarbonyl)amino)-4-(2-(1-ureidocyclobutyl)ethylsulfonimidoyl)butanoate (0.02 g, crude) as colorless oil.

A solution of (2S)-tert-butyl 2-((tert-butoxycarbonyl)amino)-4-(2-(1-ureidocyclobutyl)ethylsulfonimidoyl)butanoate (20.0 mg, 43.2 μmol) in HCl/dioxane (5 mL, 4M) was stirred at 15° C. for 5 h. The mixture was concentrated and the residue was purified by prep-HPLC: column: C18-1 150×30 mm, 5 microns; mobile phase: [water(formic acid)-MeCN]; B %: 1%-20%, 10 min to give (2S)-2-amino-4-(2-(1-ureidocyclobutyl)ethylsulfonimidoyl)butanoic acid (Compound 206) (8 mg, 48% yield, FA) as colorless oil. LCMS: Rt=0.657 min., (ES⁺) m/z (M+H)⁺=307.1; HPLC Conditions: A; ¹H NMR (400 MHz, D₂O) δ 3.94 (t, J=6.3 Hz, 1H), 3.77-3.35 (m, 4H), 2.47-2.28 (m, 4H), 2.19-1.76 (m, 6H). ¹H NMR (400 MHz, DMSO-d₆) δ 7.63-6.96 (m, 2H), 6.53-6.26 (m, 1H), 5.43 (br s, 1H), 4.24-3.04 (m, 6H), 2.91 (br t, J=8.0 Hz, 1H), 2.35-2.02 (m, 4H), 1.98-1.64 (m, 3H).

### Exemplary Embodiment 1aa8

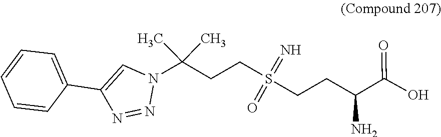 [SMILES: CC(C)(CCS(=N)(=O)CC[C@H](N)C(=O)O)n1cc(-c2ccccc2)nn1]

### (2S)-2-amino-4-(3-methyl-3-(4-phenyl-1H-1,2,3-triazol-1-yl)butylsulfonimidoyl)butanoic acid

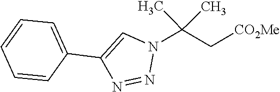 [SMILES: COC(=O)CC(C)(C)n1cc(-c2ccccc2)nn1]

To a solution of 4-phenyl-1H-1,2,3-triazole (340 mg, 2.34 mmol) in DMF (4 mL) was added DBU (927 mg, 6.09 mmol, 918 μL) and methyl 3-methylbut-2-enoate (802 mg, 7.03 mmol, 858 μL). The mixture was stirred at 25° C. for 16 h. The mixture was poured into water (5 mL) and extracted with EtOAc (5 mL×3). The combined organic extracts were dried over Na₂SO₄, filtered, and concentrated. The residue was purified by prep-TLC (SiO₂, Petroleum ether: ethyl acetate=3:1, R_{f}=0.7) to give methyl 3-methyl-3-(4-phenyl-1H-1,2,3-triazol-1-yl)butanoate (140 mg, 540 μmol, 23% yield) as a yellow oil. ¹H NMR (400 MHz, CDCl₃-d) δ 7.92-7.81 (m, 3H), 7.49-7.38 (m, 2H), 7.36-7.30 (m, 1H), 3.61 (s, 3H), 3.09 (s, 2H), 1.86 (s, 6H).

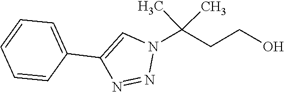 [SMILES: CC(C)(CCO)n1cc(-c2ccccc2)nn1]

To a solution of methyl 3-methyl-3-(4-phenyl-1H-1,2,3-triazol-1-yl)butanoate (160 mg, 617 μmol) in THE (2 mL) was added LAH (25.7 mg, 679 μmol) at 0° C. under N₂. The mixture was stirred at 15° C. for 2 h. The mixture was poured into water (10 mL) and extracted with EtOAc (15 mL×3). The combined organic extracts were dried over Na₂SO₄, filtered, and concentrated to give 3-methyl-3-(4-phenyl-1H-1,2,3-triazol-1-yl)butan-1-ol (120 mg, 84% yield) as a colorless oil. ¹H NMR (400 MHz, CDCl₃-d) δ 7.92-7.80 (m, 3H), 7.50-7.37 (m, 2H), 7.37-7.31 (m, 1H), 3.66 (br d, J=1.6 Hz, 2H), 2.30 (t, J=6.5 Hz, 2H), 1.78 (s, 6H).

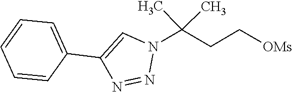 [SMILES: CC(C)(CCOS(C)(=O)=O)n1cc(-c2ccccc2)nn1]

To a solution of 3-methyl-3-(4-phenyl-1H-1,2,3-triazol-1-yl)butan-1-ol (120 mg, 519 μmol) in DCM (2 mL) was added TEA (158 mg, 1.56 mmol, 217 μL) and methanesulfonic anhydride (108 mg, 623 μmol) at 0° C. under N₂. The mixture was stirred at 15° C. for 1 h. The mixture was poured into water (10 mL) and extracted with EtOAc (15 mL×3). The combined organic extracts were dried over Na₂SO₄, filtered, and concentrated. The residue was purified by prep-TLC (SiO₂, Petroleum ether: ethyl acetate=1:1, R_{f}=0.55) to give 3-methyl-3-(4-phenyl-1H-1,2,3-triazol-1-yl)butyl methanesulfonate (80 mg, 50% yield) as a white solid. ¹H NMR (400 MHz, CDCl₃-d) δ 7.93-7.79 (m, 3H), 7.51-7.41 (m, 2H), 7.38-7.32 (m, 1H), 4.22 (t, J=6.4 Hz, 2H), 2.91 (s, 3H), 2.51 (t, J=6.4 Hz, 2H), 1.80 (s, 6H).

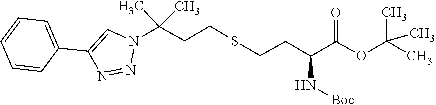 [SMILES: CC(C)(C)OC(=O)N[C@@H](CCSCCC(C)(C)n1cc(-c2ccccc2)nn1)C(=O)OC(C)(C)C]

To a solution of 3-methyl-3-(4-phenyl-1H-1,2,3-triazol-1-yl)butyl methanesulfonate (80.0 mg, 258.6 μmol) and (S)-tert-butyl 2-((tert-butoxycarbonyl)amino)-4-mercaptobutanoate (82.9 mg, 284 μmol) in DMF (2 mL) was added KI (85.8 mg, 518 μmol) and K₂CO₃ (107 mg, 776 μmol). The mixture was stirred at 30° C. for 16 h under Ar. The mixture was poured into water (10 mL) and extracted with EtOAc (15 mL×3). The combined organic extracts were dried over Na₂SO₄, filtered, and concentrated. The residue was purified by prep-TLC (SiO₂, Petroleum ether: ethyl acetate=3:1, R_{f}=0.43) to give (S)-tert-butyl 2-((tert-butoxycarbonyl)amino)-4-((3-methyl-3-(4-phenyl-1H-1,2,3-triazol-1-yl)butyl)thio)butanoate (120 mg, 92% yield) as a white solid. ¹H NMR (400 MHz, CDCl₃-d) δ 7.92-7.80 (m, 3H), 7.48-7.39 (m, 2H), 7.37-7.30 (m, 1H), 5.12 (br d, J=7.6 Hz, 1H), 4.25 (br d, J=5.3 Hz, 1H), 2.53 (t, J=7.7 Hz, 2H), 2.41-2.21 (m, 4H), 2.06-1.91 (m, 1H), 1.88-1.68 (m, 7H), 1.44 (s, 18H).

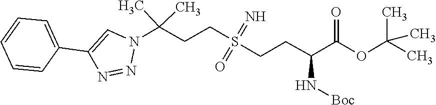 [SMILES: CC(C)(C)OC(=O)N[C@@H](CCS(=N)(=O)CCC(C)(C)n1cc(-c2ccccc2)nn1)C(=O)OC(C)(C)C]

To a solution of (S)-tert-butyl 2-((tert-butoxycarbonyl)amino)-4-((3-methyl-3-(4-phenyl-1H-1,2,3-triazol-1-yl)butyl)thio)butanoate (120 mg, 238 μmol) in i-PrOH (2 mL) was added PhI(OAc)₂ (306 mg, 952 μmol) and ammonium carbamate (149 mg, 1.90 mmol). The mixture was stirred at 15° C. for 16 h. The mixture was poured into water (10 mL) and extracted with EtOAc (15 ml×3). The combined organic extracts were dried over Na₂SO₄, filtered, and concentrated. The residue was purified by prep-TLC (SiO₂, Petroleum ether: ethyl acetate=0:1, R_{f}=0.43) to give (2S)-tert-butyl 2-((tert-butoxycarbonyl)amino)-4-(3-methyl-3-(4-phenyl-1H-1,2,3-triazol-1-yl)butylsulfonimidoyl)butanoate (96 mg, 75% yield) as a white solid. ¹H NMR (400 MHz, CDCl₃-d) δ 7.89-7.80 (m, 3H), 7.48-7.40 (m, 2H), 7.39-7.31 (m, 1H), 5.39-5.27 (m, 1H), 4.32-4.19 (m, 1H), 3.19-3.02 (m, 2H), 3.00-2.85 (m, 2H), 2.66-2.47 (m, 2H), 2.39-2.27 (m, 1H), 2.11-1.97 (m, 1H), 1.78 (s, 6H), 1.45 (d, J=7.5 Hz, 18H).

A solution of (2S)-tert-butyl 2-((tert-butoxycarbonyl)amino)-4-(3-methyl-3-(4-phenyl-1H-1,2,3-triazol-1-yl)butylsulfonimidoyl)butanoate (90 mg, 168 μmol) in HCl/dioxane (4 M, 2 mL) was stirred at 25° C. for 16 h. The reaction mixture was concentrated. The mixture was poured into water (1 mL) and extracted with DCM (1 ml×3). The aqueous phase was concentrated to give (2S)-2-amino-4-(3-methyl-3-(4-phenyl-1H-1,2,3-triazol-1-yl)butylsulfonimidoyl)butanoic acid (Compound 207) (43 mg, 62% yield, HCl) as a white solid. LCMS: Rt=2.929 min, (ES⁺) m/z (M+H)⁺=380.1; HPLC Conditions: A; ¹H NMR (400 MHz, D₂O) δ 8.47 (s, 1H), 7.76 (d, J=7.1 Hz, 2H), 7.53-7.39 (m, 3H), 4.18 (ddd, J=2.1, 5.7, 7.7 Hz, 1H), 4.08-3.82 (m, 2H), 3.75-3.57 (m, 2H), 2.73-2.58 (m, 2H), 2.56-2.36 (m, 2H), 1.75 (s, 6H).

### Exemplary Embodiment 1aa9

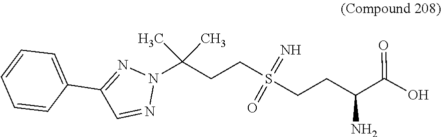 [SMILES: CC(C)(CCS(=N)(=O)CC[C@H](N)C(=O)O)n1ncc(-c2ccccc2)n1]

### (2S)-2-amino-4-(3-methyl-3-(4-phenyl-2H-1,2,3-triazol-2-yl)butylsulfonimidoyl)butanoic acid

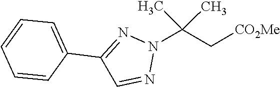 [SMILES: COC(=O)CC(C)(C)n1ncc(-c2ccccc2)n1]

To a solution of 4-phenyl-1H-triazole (100 mg, 689 μmol) in DMF (2 mL) was added DBU (273 mg, 1.79 mmol, 270 μL) and methyl 3-methylbut-2-enoate (189 mg, 1.65 mmol, 202 μL). The mixture was stirred at 40° C. for 16 h. The mixture was poured into water (10 mL) and extracted with EtOAc (15 mL×3). The combined organic extracts were dried over Na₂SO₄, filtered, and concentrated. The residue was purified by prep-HPLC (column: Phenomenex Luna C18 75×30 mm×3 um; mobile phase: [water(formic acid)-MeCN]; B %: 20%-80%, 8 min) to give methyl 3-methyl-3-(4-phenyl-2H-1,2,3-triazol-2-yl)butanoate (100 mg, 20% yield) as a colorless oil. ¹H NMR (400 MHz, CDCl₃-d) δ 7.90-7.74 (m, 3H), 7.50-7.39 (m, 2H), 7.37-7.30 (m, 1H), 3.62 (s, 3H), 3.07 (s, 2H), 1.85 (s, 6H).

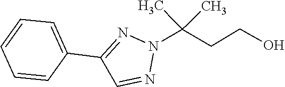 [SMILES: CC(C)(CCO)n1ncc(-c2ccccc2)n1]

To a solution of methyl 3-methyl-3-(4-phenyl-2H-1,2,3-triazol-2-yl)butanoate (220 mg, 848 μmol) in THE (2 mL) was added LiAlH₄ (38.6 mg, 1.02 mmol) at 0° C. The mixture was stirred at 20° C. for 1 h. The reaction mixture was diluted with H₂O (20 mL), extracted with EtOAc (15 mL×3). The combined organic phase was washed with brine (30 mL), dried with anhydrous Na₂SO₄, filtered, and concentrated to give 3-methyl-3-(4-phenyl-2H-1,2,3-triazol-2-yl)butan-1-ol (180 mg, 83% yield) as a colorless oil. ¹H NMR (400 MHz, CDCl₃-d) δ 7.84 (s, 1H), 7.82-7.76 (m, 2H), 7.45-7.33 (m, 3H), 3.69 (t, J=6.4 Hz, 2H), 2.31 (t, J=6.4 Hz, 2H), 1.77 (s, 6H).

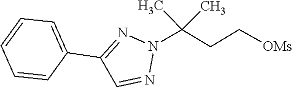 [SMILES: CC(C)(CCOS(C)(=O)=O)n1ncc(-c2ccccc2)n1]

To a solution of 3-methyl-3-(4-phenyl-2H-1,2,3-triazol-2-yl)butan-1-ol (170 mg, 735 μmol) and TEA (112 mg, 1.10 mmol, 153 μL) in DCM (2 mL) was added methanesulfonic anhydride (154 mg, 882 μmol) in portions at 0° C. under N₂. The mixture was warmed to 20° C. and stirred at 20° C. for 2 h. The reaction mixture was quenched by addition water (8 mL) at 0° C., and extracted with DCM (15 mL×2). The combined organic extracts were dried over Na₂SO₄, filtered, and concentrated. The residue was purified by column chromatography (SiO₂, Petroleum ether/Ethyl acetate=5/1 to 1/3) to afford 3-methyl-3-(4-phenyl-2H-1,2,3-triazol-2-yl)butyl methanesulfonate (160 mg, 65% yield) as colorless oil.

¹H NMR (400 MHz, CDCl₃-d) δ 7.85 (s, 1H), 7.80 (d, J=7.9 Hz, 2H), 7.47-7.37 (m, 3H), 4.21 (t, J=6.8 Hz, 2H), 2.94 (s, 3H), 2.51 (t, J=6.8 Hz, 2H), 1.79 (s, 6H).

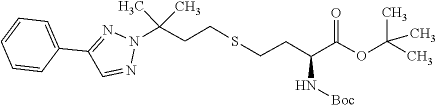 [SMILES: CC(C)(C)OC(=O)N[C@@H](CCSCCC(C)(C)n1ncc(-c2ccccc2)n1)C(=O)OC(C)(C)C]

To a solution of 3-methyl-3-(4-phenyl-2H-1,2,3-triazol-2-yl)butyl methanesulfonate (150 mg, 485 μmol) in DMF (2 mL) was added (S)-tert-butyl 2-((tert-butoxycarbonyl)amino)-4-mercaptobutanoate (141 mg, 485 μmol), K₂CO₃ (201 mg, 1.45 mmol) and KI (161 mg, 970 μmol). The mixture was stirred at 40° C. for 16 h. The reaction mixture was diluted with H₂O (30 mL) and extracted with EtOAc (20 mL×3). The combined organic phase was washed with brine (30 mL), dried with anhydrous Na₂SO₄, filtered, and concentrated to give a residue. The residue was purified by column chromatography (SiO₂, Petroleum ether/Ethyl acetate=1/0 to 1/1) to give (S)-tert-butyl 2-((tert-butoxycarbonyl)amino)-4-((3-methyl-3-(4-phenyl-2H-1,2,3-triazol-2-yl)butyl)thio)butanoate (230 mg, 80% yield) as colorless oil. ¹H NMR (400 MHz, CDCl₃-d) δ 7.84 (s, 1H), 7.83-7.76 (m, 2H), 7.47-7.40 (m, 2H), 7.36-7.32 (m, 1H), 5.18-5.03 (m, 1H), 4.34-4.18 (m, 1H), 2.53 (t, J=8.0 Hz, 2H), 2.37-2.25 (m, 4H), 2.06-1.99 (m, 1H), 1.87-1.79 (m, 1H), 1.73 (d, J=2.7 Hz, 6H), 1.46-1.43 (m, 18H).

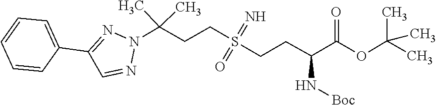 [SMILES: CC(C)(C)OC(=O)N[C@@H](CCS(=N)(=O)CCC(C)(C)n1ncc(-c2ccccc2)n1)C(=O)OC(C)(C)C]

To a solution of (S)-tert-butyl 2-((tert-butoxycarbonyl)amino)-4-((3-methyl-3-(4-phenyl-2H-1,2,3-triazol-2-yl)butyl)thio)butanoate (230 mg, 456 μmol) in i-PrOH (3 mL) was added PhI(OAc)₂ (587 mg, 1.82 mmol) and ammonium carbamate (285 mg, 3.65 mmol). The mixture was stirred at 25° C. for 16 h. The reaction mixture was concentrated. The reaction mixture was diluted with H₂O (20 mL) and extracted with DCM (15 mL×3). The combined organic phase was washed with brine (30 mL), dried with anhydrous Na₂SO₄, filtered, and concentrated to give a residue. The residue was purified by column chromatography (SiO₂, Petroleum ether/Ethyl acetate=1/0 to 1/2) to give (2S)-tert-butyl 2-((tert-butoxycarbonyl)amino)-4-(3-methyl-3-(4-phenyl-2H-1,2,3-triazol-2-yl)butylsulfonimidoyl)butanoate (150 mg, 57% yield), as colorless oil. ¹H NMR (400 MHz, CDCl₃-d) δ 7.87 (s, 1H), 7.83-7.76 (m, 2H), 7.47-7.41 (m, 2H), 7.39-7.33 (m, 1H), 5.22 (br d, J=3.6 Hz, 1H), 4.35-4.20 (m, 1H), 3.24-2.99 (m, 2H), 2.98-2.87 (m, 2H), 2.61-2.51 (m, 2H), 2.41-2.27 (m, 1H), 2.13-2.06 (m, 1H), 1.77 (s, 6H), 1.45 (d, J=7.9 Hz, 18H).

A solution of (2S)-tert-butyl 2-((tert-butoxycarbonyl)amino)-4-(3-methyl-3-(4-phenyl-2H-1,2,3-triazol-2-yl)butylsulfonimidoyl)butanoate (60.0 mg, 112 μmol) in HCl/dioxane (4 M, 28.0 μL) was stirred at 25° C. for 12 h. The reaction mixture was concentrated and the residue was purified by prep-HPLC (column: Phenomenex Luna C18 200×40 mm×10 um; mobile phase: [water(FA)-MeCN]; B %: 15%-55%, 8 min) to give (2S)-2-amino-4-(3-methyl-3-(4-phenyl-2H-1,2,3-triazol-2-yl)butylsulfonimidoyl)butanoic acid (Compound 208) (18.0 mg, 42% yield) as a white solid. LCMS: Rt=1.971 min, (ES⁺) m/z (M+H)⁺=380.1; HPLC Conditions C; ¹H NMR (400 MHz, D₂O) δ 8.09 (s, 1H), 7.81 (br d, J=7.3 Hz, 2H), 7.60-7.38 (m, 3H), 3.81-3.71 (m, 1H), 3.42-3.21 (m, 2H), 3.14-3.04 (m, 2H), 2.58-2.47 (m, 2H), 2.32-2.19 (m, 2H), 1.74 (s, 6H).

### Exemplary Embodiment 1aa10

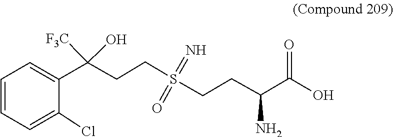 [SMILES: N=S(=O)(CC[C@H](N)C(=O)O)CCC(O)(c1ccccc1Cl)C(F)(F)F]

### (2S)-2-amino-4-(3-(2-chlorophenyl)-4,4,4-trifluoro-3-hydroxybutylsulfonimidoyl)butanoic acid

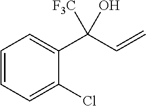

To a solution of 1-(2-chlorophenyl)-2,2,2-trifluoro-ethanone (500 mg, 2.40 mmol) in THE (5 mL) was added chloro(vinyl)magnesium (2 M, 1.32 mL) slowly at −65° C. The mixture was stirred at 0° C. for 1 h. The reaction mixture was diluted with H₂O (10 mL) and extracted with ethyl acetate (5 mL×3). The combined organic phase was washed with brine (10 mL), dried with anhydrous Na₂SO₄, filtered, and concentrated. The residue was purified by prep-TLC (Petroleum ether: ethyl acetate=10:1) to give 2-(2-chlorophenyl)-1,1,1-trifluorobut-3-en-2-ol (400 mg, 71% yield) as a yellow oil. ¹H NMR (400 MHz, CDCl₃-d) δ 7.81-7.73 (m, 1H), 7.45-7.38 (m, 1H), 7.37-7.29 (m, 2H), 6.55 (dd, J=10.8, 17.4 Hz, 1H), 5.61-5.51 (m, 2H).

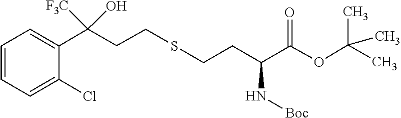 [SMILES: CC(C)(C)OC(=O)N[C@@H](CCSCCC(O)(c1ccccc1Cl)C(F)(F)F)C(=O)OC(C)(C)C]

(2S)-tert-butyl 2-((tert-butoxycarbonyl)amino)-4-((3-(2-chlorophenyl)-4,4,4-trifluoro-3-hydroxybutyl)thio)butanoate: A mixture of 2-(2-chlorophenyl)-1,1,1-trifluorobut-3-en-2-ol (391 mg, 1.65 mmol), (S)-tert-butyl 2-((tert-butoxycarbonyl)amino)-4-mercaptobutanoate (370 mg, 1.27 mmol) and AIBN (20.9 mg, 127 μmol) in H₂O (6 mL) and MeOH (6 mL) was stirred at 60° C. for 12 h under Ar. The reaction mixture was concentrated. The residue mixture was diluted with H₂O (5 mL), extracted with ethyl acetate (5 mL×3). The combined organic phase was washed with brine (5 mL), dried with anhydrous Na₂SO₄, filtered, and concentrated. The residue was purified by prep-TLC (SiO₂, Petroleum ether: ethyl acetate=5:1) to give (2S)-tert-butyl 2-((tert-butoxycarbonyl)amino)-4-((3-(2-chlorophenyl)-4,4,4-trifluoro-3-hydroxybutyl)thio)butanoate (197 mg, 29% yield) as a yellow oil. ¹H NMR (400 MHz, CDCl₃-d) δ 7.87 (br d, J=7.5 Hz, 1H), 7.44-7.39 (m, 1H), 7.34 (tt, J=2.4, 7.7 Hz, 2H), 5.22-5.07 (m, 1H), 4.62-4.51 (m, 1H), 4.35-4.22 (m, 1H), 3.26-3.11 (m, 1H), 2.60-2.51 (m, 4H), 2.36-2.27 (m, 1H), 1.93-1.78 (m, 1H), 1.71-1.60 (m, 1H), 1.47-1.45 (m, 18H).

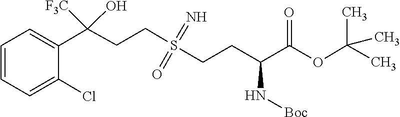 [SMILES: CC(C)(C)OC(=O)N[C@@H](CCS(=N)(=O)CCC(O)(c1ccccc1Cl)C(F)(F)F)C(=O)OC(C)(C)C]

(2S)-tert-butyl 2-((tert-butoxycarbonyl)amino)-4-(3-(2-chlorophenyl)-4,4,4-trifluoro-3-hydroxybutylsulfonimidoyl)butanoate: A mixture of (2S)-tert-butyl 2-((tert-butoxycarbonyl)amino)-4-((3-(2-chlorophenyl)-4,4,4-trifluoro-3-hydroxybutyl)thio)butanoate (197 mg, 373 μmol), PhI(OAc)₂ (481 mg, 1.49 mmol) and ammonium carbamate (233 mg, 2.98 mmol) in i-PrOH (3 mL) was stirred at 25° C. for 16 h. The reaction mixture was concentrated and the residue was diluted with water (10 mL) and extracted with Ethyl acetate (5 mL×2). The combined organic extracts were washed with brine (10 mL), dried over Na₂SO₄, filtered, and concentrated. The residue was purified by prep-TLC (Petroleum ether: ethyl acetate=1:1) to give (2S)-tert-butyl 2-((tert-butoxycarbonyl)amino)-4-(3-(2-chlorophenyl)-4,4,4-trifluoro-3-hydroxybutylsulfonimidoyl)butanoate (120 mg, 58% yield) as a yellow oil.

(2S)-2-amino-4-(3-(2-chlorophenyl)-4,4,4-trifluoro-3-hydroxybutylsulfonimidoyl)butanoic acid: A mixture of (2S)-tert-butyl 2-((tert-butoxycarbonyl)amino)-4-(3-(2-chlorophenyl)-4,4,4-trifluoro-3-hydroxybutylsulfonimidoyl)butanoate (60 mg, 107 μmol) in HCl/dioxane (5 mL) was stirred at 20° C. for 12 h. The reaction mixture was concentrated and the residue was purified by prep-HPLC (column: C18-1 150×30 mm, 5 microns; mobile phase: [water (FA)-MeCN]; B %: 10%-40%, 10 min) to give (2S)-2-amino-4-(3-(2-chlorophenyl)-4,4,4-trifluoro-3-hydroxybutylsulfonimidoyl)butanoic acid (Compound 209) (31.1 mg, 72% yield) as a white solid.

LCMS: Rt=3.123 min., (ES⁺) m/z (M+H)⁺=402.9; HPLC Conditions: D; ¹H NMR (400 MHz, D₂O) δ 7.85-7.76 (m, 1H), 7.57-7.48 (m, 1H), 7.47-7.37 (m, 2H), 3.88-3.77 (m, 1H), 3.50-3.05 (m, 5H), 2.65-2.53 (m, 1H), 2.34-2.24 (m, 2H).

### Exemplary Embodiment 1aa11

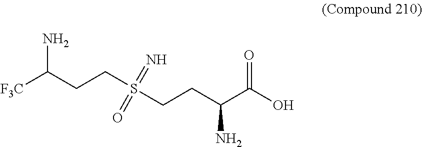 [SMILES: N=S(=O)(CCC(N)C(F)(F)F)CC[C@H](N)C(=O)O]

### (2S)-2-amino-4-(3-amino-4,4,4-trifluorobutylsulfonimidoyl)butanoic acid

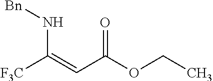 [SMILES: CCOC(=O)/C=C(\NCc1ccccc1)C(F)(F)F]

To a solution of BnNH₂ (1.45 g, 13.5 mmol, 1.48 mL) in CHCl₃ (5.5 mL) was added AcOH (0.8 mL). After stirring for 5 min, ethyl 4,4,4-trifluoro-3-oxobutanoate (2.27 g, 12.3 mmol, 1.8 mL) was added. The mixture was stirred at 100° C. for 16 h. The reaction mixture was poured into H₂O (30 mL) and the aqueous phase was extracted with ethyl acetate (30 mL×2). The combined organic phase was washed with brine (30 mL), dried with anhydrous Na₂SO₄, filtered, and concentrated. The residue was purified by silica gel chromatography (Petroleum ether/Ethyl acetate=5/1) to give (Z)-ethyl 3-(benzylamino)-4,4,4-trifluorobut-2-enoate (3 g, 89% yield) as colorless oil. ¹H NMR (400 MHz, CDCl₃-d) δ 8.45 (br s, 1H), 7.43-7.28 (m, 5H), 5.18 (s, 1H), 4.49 (d, J=6.3 Hz, 2H), 4.15 (q, J=7.2 Hz, 2H), 1.28 (t, J=7.1 Hz, 3H).

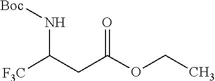 [SMILES: CCOC(=O)CC(NC(=O)OC(C)(C)C)C(F)(F)F]

To a solution of (Z)-ethyl 3-(benzylamino)-4,4,4-trifluorobut-2-enoate (2.40 g, 8.78 mmol) in MeOH (5 mL) was added Boc₂O (19.2 g, 87.8 mmol, 20.1 mL), Pd/C (8.78 mmol, 10% purity), and the mixture was stirred at 20° C. for 16 h. The reaction mixture was poured into H₂O (30 mL) and extracted with ethyl acetate (30 mL×2). The combined organic phase was washed with brine (30 mL), dried with anhydrous Na₂SO₄, filtered, and concentrated. The residue was purified by silica gel chromatography (Petroleum ether/Ethyl acetate=5/1) to give ethyl 3-((tert-butoxycarbonyl)amino)-4,4,4-trifluorobutanoate (1.5 g, 60% yield) as a colorless oil. ¹H NMR (400 MHz, CDCl₃-d) δ 5.27-5.16 (m, 1H), 4.77-4.63 (m, 1H), 4.20 (q, J=7.2 Hz, 2H), 2.81-2.71 (m, 1H), 2.64-2.52 (m, 1H), 1.46 (s, 9H), 1.28 (t, J=7.2 Hz, 4H).

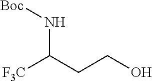 [SMILES: CC(C)(C)OC(=O)NC(CCO)C(F)(F)F]

To a solution of ethyl 3-((tert-butoxycarbonyl)amino)-4,4,4-trifluorobutanoate (1.00 g, 3.51 mmol) in THE (8 mL) was added LiAlH₄ (159 mg, 4.21 mmol) at 0° C. under N₂. The mixture was stirred at 20° C. for 2 h. The reaction mixture was poured into H₂O (50 mL) and the aqueous phase was extracted with ethyl acetate (50 mL×2). The combined organic phase was washed with brine (50 mL), dried with anhydrous Na₂SO₄, filtered, and concentrated. The residue was purified by silica gel chromatography (Petroleum ether/Ethyl acetate=5/1) to give tert-butyl (1,1,1-trifluoro-4-hydroxybutan-2-yl)carbamate (700 mg, 82% yield) as colorless oil. ¹H NMR (400 MHz, CDCl₃-d) δ 4.79 (br d, J=9.0 Hz, 1H), 4.54-4.37 (m, 1H), 3.83-3.65 (m, 2H), 2.57 (br dd, J=5.4, 7.0 Hz, 1H), 2.16-2.07 (m, 1H), 1.64-1.55 (m, 2H), 1.48 (s, 9H).

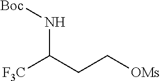 [SMILES: CC(C)(C)OC(=O)NC(CCOS(C)(=O)=O)C(F)(F)F]

To a solution of tert-butyl (1,1,1-trifluoro-4-hydroxybutan-2-yl)carbamate (700 mg, 2.88 mmol) in DCM (3 mL) was added methylsulfonyl methanesulfonate (601 mg, 3.45 mmol) and TEA (582 mg, 5.76 mmol, 801 μL) at 0° C. under N₂. The mixture was stirred at 20° C. for 2 h. The reaction mixture was poured into H₂O (30 mL) and the aqueous phase was extracted with DCM (30 mL×2). The combined organic phase was washed with brine (50 mL), dried with anhydrous Na₂SO₄, filtered and concentrated. The residue was purified by silica gel chromatography (Petroleum ether/Ethyl acetate=5/1) to give 3-((tert-butoxycarbonyl)amino)-4,4,4-trifluorobutyl methanesulfonate (600 mg, 65% yield) as colorless oil. ¹H NMR (400 MHz, CDCl₃-d) δ 4.71-4.62 (m, 1H), 4.51-4.41 (m, 1H), 4.40-4.25 (m, 2H), 3.06 (s, 3H), 2.36-2.24 (m, 1H), 1.93-1.80 (m, 1H), 1.47 (s, 9H).

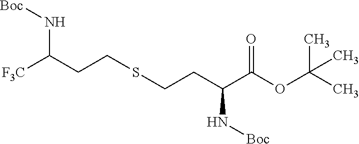 [SMILES: CC(C)(C)OC(=O)NC(CCSCC[C@H](NC(=O)OC(C)(C)C)C(=O)OC(C)(C)C)C(F)(F)F]

To a solution of 3-((tert-butoxycarbonyl)amino)-4,4,4-trifluorobutyl methanesulfonate (303 mg, 943 μmol, 1.1 eq) in DMF (5 mL) was added tert-butyl (2S)-2-(tert-butoxycarbonylamino)-4-sulfanyl-butanoate (250 mg, 857.91 μmol), KI (284 mg, 1.72 mmol) and K₂CO₃ (355 mg, 2.57 mmol) in a glove box. The mixture was stirred at 50° C. for 16 h. The reaction mixture was poured into H₂O (30 mL) and extracted with ethyl acetate (30 mL×2). The combined organic phase was washed with brine (30 mL), dried with anhydrous Na₂SO₄, filtered, and concentrated. The residue was purified by silica gel chromatography (Petroleum ether/Ethyl acetate=3/1) to give (2S)-tert-butyl 2-((tert-butoxycarbonyl)amino)-4-((3-((tert-butoxycarbonyl)amino)-4,4,4-trifluorobutyl)thio)butanoate (400 mg, 90.3% yield).

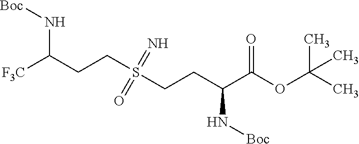 [SMILES: CC(C)(C)OC(=O)NC(CCS(=N)(=O)CC[C@H](NC(=O)OC(C)(C)C)C(=O)OC(C)(C)C)C(F)(F)F]

A mixture of (2S)-tert-butyl 2-((tert-butoxycarbonyl)amino)-4-((3-((tert-butoxycarbonyl)amino)-4,4,4-trifluorobutyl)thio)butanoate (200 mg, 387 μmol), PhI(OAc)₂ (311 mg, 967 μmol) and ammonium carbamate (151 mg, 1.94 mmol) in i-PrOH (5 mL) was stirred at 20° C. for 3 h. The reaction mixture was poured into H₂O (30 mL) and extracted with ethyl acetate (30 mL×2). The combined organic phase was washed with brine (30 mL), dried with anhydrous Na₂SO₄, filtered, and concentrated. The residue was purified by silica gel chromatography (Petroleum ether/Ethyl acetate=1/1) to give (2S)-tert-butyl 2-((tert-butoxycarbonyl)amino)-4-(3-((tert-butoxycarbonyl)amino)-4,4,4-trifluorobutylsulfonimidoyl)butanoate (120 mg, 57% yield) as colorless oil. ¹H NMR (400 MHz, CDCl₃-d) δ 5.31 (br s, 1H), 5.03-4.81 (m, 1H), 4.42-4.22 (m, 2H), 3.28-2.98 (m, 4H), 2.49-2.28 (m, 2H), 2.23-2.07 (m, 1H), 1.54-1.41 (m, 27H).

A solution of (2S)-tert-butyl 2-((tert-butoxycarbonyl)amino)-4-(3-((tert-butoxycarbonyl)amino)-4,4,4-trifluorobutylsulfonimidoyl)butanoate (120 mg, 219 μmol) in HCl/dioxane (4 mL) was stirred at 20° C. for 16 h. The reaction mixture was concentrated and the residue was purified by prep-HPLC (column: C18-1 150×30 mm, 5 microns; mobile phase: [water(FA)-MeCN]; B %: 1%-10%, 10 min) to give (2S)-2-amino-4-(3-amino-4,4,4-trifluorobutylsulfonimidoyl)butanoic acid (Compound 210) (33 mg, 52% yield, HCl) as white solid. LCMS: Rt=0.272 min, (ES⁺) m/z (M+H)⁺=292.0; HPLC Conditions: A; ¹H NMR (400 MHz, D₂O) δ 4.45-4.29 (m, 1H), 4.06 (br t, J=6.1 Hz, 1H), 3.69-3.48 (m, 4H), 2.60-2.49 (m, 1H), 2.47-2.33 (m, 3H).

### Exemplary Embodiment 1aa12

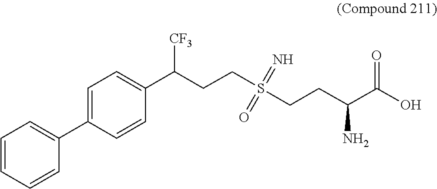 [SMILES: N=S(=O)(CCC(c1ccc(-c2ccccc2)cc1)C(F)(F)F)CC[C@H](N)C(=O)O]

### (2S)-4-(3-([1,1′-biphenyl]-4-yl)-4,4,4-trifluorobutylsulfonimidoyl)-2-aminobutanoic acid

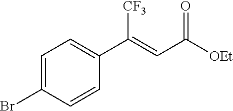 [SMILES: CCOC(=O)/C=C(/c1ccc(Br)cc1)C(F)(F)F]

To a solution of ethyl 2-diethoxyphosphorylacetate (2.30 g, 10.2 mmol, 2.04 mL) in THE (20 mL) was added NaH (474 mg, 11.8 mmol, 60%) at 0° C. and the mixture was stirred at 15° C. for 0.5 h under N₂. 1-(4-Bromophenyl)-2,2,2-trifluoroethanone (2 g, 7.90 mmol, 1.20 mL, 1 eq) was added and the mixture was stirred at 15° C. for 2.5 hr under N₂. The mixture was poured into water (10 mL) and extracted with EtOAc (15 mL×3). The combined organic extracts were dried over Na₂SO₄, filtered, and concentrated. The residue was purified by column chromatography (SiO₂, Petroleum ether: ethyl acetate=90:10) to give ethyl 3-(4-bromophenyl)-4,4,4-trifluorobut-2-enoate (2.2 g, 86% yield) as a colorless oil. ¹H NMR (400 MHz, CDCl₃-d) δ 7.55 (d, J=8.4 Hz, 2H), 7.17 (d, J=8.3 Hz, 2H), 6.63 (d, J=1.1 Hz, 1H), 4.08 (q, J=7.1 Hz, 2H), 1.12 (t, J=7.2 Hz, 3H).

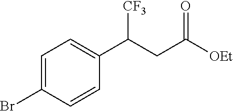 [SMILES: CCOC(=O)CC(c1ccc(Br)cc1)C(F)(F)F]

To a solution of ethyl 3-(4-bromophenyl)-4,4,4-trifluorobut-2-enoate (1.30 g, 4.02 mmol) in MeOH (10 mL) was added COCl₂·6H₂O (95.7 mg, 402 μmol) and NaBH₄ (228 mg, 6.04 mmol) at 0° C. under Ar. The mixture was stirred at 15° C. for 24 h and then the mixture was poured into water (20 mL) and extracted with EtOAc (15 mL×3). The combined organic layers were dried over Na₂SO₄, filtered, and concentrated to give ethyl 3-(4-bromophenyl)-4,4,4-trifluorobutanoate (1.19 g, 91% yield) as a colorless oil. ¹H NMR (400 MHz, CDCl₃-d) δ 7.53-7.46 (m, 2H), 7.22 (d, J=8.2 Hz, 2H), 4.19-3.99 (m, 2H), 3.89 (dquin, J=5.1, 9.4 Hz, 1H), 3.09-2.79 (m, 2H), 1.23-1.11 (m, 3H).

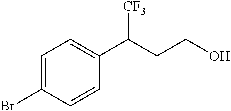 [SMILES: OCCC(c1ccc(Br)cc1)C(F)(F)F]

To a solution of ethyl 3-(4-bromophenyl)-4,4,4-trifluorobutanoate (1.10 g, 3.38 mmol) in THE (10 mL) was added LAH (154 mg, 4.06 mmol) at −40° C. under N₂. The mixture was stirred at −40° C. for 1 hr. The mixture was diluted with ethyl acetate (10 mL), quenched with water (0.15 mL), 15% NaOH (0.15 mL) and water (0.46 mL). The mixture was filtered and the filtrate was concentrated. The residue was purified by column chromatography (SiO₂, Petroleum ether: ethyl acetate=87:13) to give 3-(4-bromophenyl)-4,4,4-trifluorobutan-1-ol (620 mg, 65% yield) as a colorless oil. ¹H NMR (400 MHz, CDCl₃-d) δ 7.41-7.30 (m, 4H), 3.75-3.64 (m, 1H), 3.63-3.50 (m, 1H), 3.47-3.34 (m, 1H), 2.37-2.24 (m, 1H), 2.15-2.06 (m, 1H).

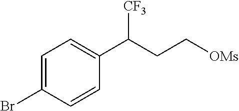 [SMILES: CS(=O)(=O)OCCC(c1ccc(Br)cc1)C(F)(F)F]

To a solution of 3-(4-bromophenyl)-4,4,4-trifluorobutan-1-ol (600 mg, 2.12 mmol) in DCM (6 mL) was added methylsulfonyl methanesulfonate (443 mg, 2.54 mmol) and TEA (643 mg, 6.36 mmol, 885 μL) at 0° C. The mixture was stirred at 20° C. for 1 hr under N₂. The mixture was poured into water (10 mL) and extracted with EtOAc (15 mL×3). The combined organic extracts were dried over Na₂SO₄, filtered, and concentrated under reduced pressure to give: 3-(4-bromophenyl)-4,4,4-trifluorobutyl methanesulfonate (670 mg, 88% yield) as a yellow oil. ¹H NMR (400 MHz, CDCl₃-d) δ 7.44-7.36 (m, 2H), 7.35-7.29 (m, 2H), 4.32-4.19 (m, 1H), 3.96 (dt, J=4.8, 9.8 Hz, 1H), 3.61-3.46 (m, 1H), 2.98-2.89 (m, 3H), 2.59-2.47 (m, 1H), 2.33-2.18 (m, 1H).

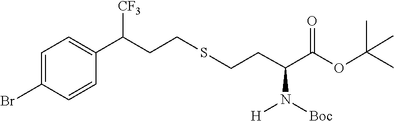 [SMILES: CC(C)(C)OC(=O)N[C@@H](CCSCCC(c1ccc(Br)cc1)C(F)(F)F)C(=O)OC(C)(C)C]

To a solution of: 3-(4-bromophenyl)-4,4,4-trifluorobutyl methanesulfonate (670 mg, 1.86 mmol) and tert-butyl (tert-butoxycarbonyl)-L-homocysteinate (540 mg, 1.86 mmol) in DMF (10 mL) was added K₂CO₃ (769 mg, 5.57 mmol) and KI (615 mg, 3.71 mmol). The mixture was stirred at 40° C. for 16 hr under Ar. The mixture was poured into water (20 mL) and extracted with EtOAc (25 mL×3). The combined organic layers were dried over Na₂SO₄, filtered, and concentrated under reduced pressure. The residue was purified by prep-HPLC (column: Phenomenex Luna C18 75×30 mm×3 um; mobile phase: [water(FA)-ACN]; B %: 40%-80%, 8 min) to give (2S)-tert-butyl 4-((3-(4-bromophenyl)-4,4,4-trifluorobutyl)thio)-2-((tert-butoxycarbonyl)amino)butanoate (200 mg, 19% yield) as yellow oil. ¹H NMR (400 MHz, CDCl₃-d) δ 7.51 (d, J=8.4 Hz, 2H), 7.19 (br d, J=7.5 Hz, 2H), 5.07 (br s, 1H), 4.33-4.17 (m, 1H), 3.64-3.44 (m, 1H), 2.62-2.39 (m, 3H), 2.31-1.96 (m, 4H), 1.91-1.73 (m, 1H), 1.57-1.41 (m, 18H).

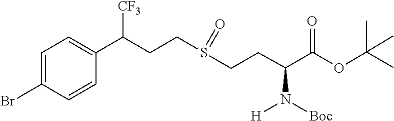 [SMILES: CC(C)(C)OC(=O)N[C@@H](CCS(=O)CCC(c1ccc(Br)cc1)C(F)(F)F)C(=O)OC(C)(C)C]

To a solution of (2S)-tert-butyl 4-((3-(4-bromophenyl)-4,4,4-trifluorobutyl)thio)-2-((tert-butoxycarbonyl)amino)butanoate (200 mg, 359 μmol) in DCM (3 mL) was added m-CPBA (72.9 mg, 359 μmol, 85%) at 0° C. The mixture was stirred at 15° C. for 1 hr. The mixture was poured into water (10 mL) and extracted with DCM (15 mL×3). The combined organic layers were dried over Na₂SO₄, filtered, and concentrated under reduced pressure. The residue was purified by prep-TLC (SiO₂, Petroleum ether: ethyl acetate=1:1) to give (2S)-tert-butyl 4-((3-(4-bromophenyl)-4,4,4-trifluorobutyl)sulfinyl)-2-((tert-butoxycarbonyl)amino)butanoate (150 mg, 73% yield) as colorless oil. ¹H NMR (400 MHz, CDCl₃-d) δ 7.53 (br d, J=8.1 Hz, 2H), 7.19 (br dd, J=2.4, 5.7 Hz, 2H), 5.27-5.11 (m, 1H), 4.34-4.19 (m, 1H), 3.55-3.38 (m, 1H), 2.79-2.63 (m, 2H), 2.61-2.42 (m, 3H), 2.39-2.20 (m, 2H), 2.02-1.90 (m, 1H), 1.56-1.36 (m, 18H).

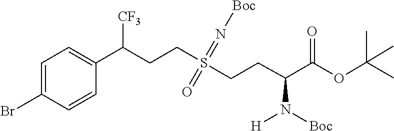 [SMILES: CC(C)(C)OC(=O)N=S(=O)(CCC(c1ccc(Br)cc1)C(F)(F)F)CC[C@H](NC(=O)OC(C)(C)C)C(=O)OC(C)(C)C]

To a solution of (2S)-tert-butyl 4-((3-(4-bromophenyl)-4,4,4-trifluorobutyl)sulfinyl)-2-((tert-butoxycarbonyl)amino)butanoate (150 mg, 262 μmol) in DCM (4 mL) was added NH₂Boc (61.3 mg, 524 μmol), PhI(OAc)₂ (168 mg, 524 μmol), MgO (42.2 mg, 1.05 mmol, 11.8 μL) and diacetoxyrhodium (5.79 mg, 13.1 μmol). The mixture was stirred at 40° C. for 16 hr. The mixture was filtered, and the filtrate was concentrated under reduced pressure. The mixture was poured into water (5 mL) and extracted with EtOAc (5 mL×3). The combined organic layers were dried over Na₂SO₄, filtered, and concentrated. The residue was purified by prep-TLC (SiO₂, Petroleum ether: ethyl acetate=3:1) to give (2S)-tert-butyl 4-(3-(4-bromophenyl)-N-(tert-butoxycarbonyl)-4,4,4-trifluorobutylsulfonimidoyl)-2-((tert-butoxycarbonyl)amino)butanoate (120 mg, 67% yield), as colorless oil. ¹H NMR (400 MHz, CDCl₃-d) δ 7.58-7.51 (m, 2H), 7.19 (br d, J=7.8 Hz, 2H), 5.20 (br d, J=1.0 Hz, 1H), 4.19 (br d, J=2.6 Hz, 1H), 3.59-2.98 (m, 5H), 2.58 (tdd, J=4.7, 9.1, 14.0 Hz, 1H), 2.43-2.25 (m, 2H), 2.05-1.95 (m, 1H), 1.51-1.39 (m, 27H).

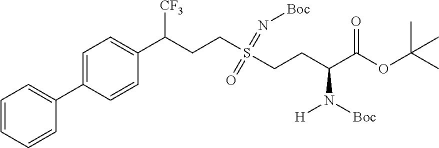 [SMILES: CC(C)(C)OC(=O)N=S(=O)(CCC(c1ccc(-c2ccccc2)cc1)C(F)(F)F)CC[C@H](NC(=O)OC(C)(C)C)C(=O)OC(C)(C)C]

To a solution of (2S)-tert-butyl 4-(3-(4-bromophenyl)-N-(tert-butoxycarbonyl)-4,4,4-trifluorobutylsulfonimidoyl)-2-((tert-butoxycarbonyl)amino)butanoate (60.0 mg, 87.2 μmol, 1 eq) and phenylboronic acid (12.7 mg, 104 μmol) in dioxane (3 mL) and H₂O (0.6 mL) was added K₂CO₃ (36.1 mg, 261 μmol) and Pd(dppf)Cl₂ (3.19 mg, 4.36 μmol) under N₂. The mixture was stirred at 100° C. for 7 h under N₂. The mixture was poured into water (5 mL) and extracted with EtOAc (5 mL×3). The combined organic layers were dried over Na₂SO₄, filtered, and concentrated under reduced pressure. The residue was purified by prep-TLC (SiO₂, Petroleum ether: ethyl acetate=3:1) to give (2S)-tert-butyl 4-(3-([1,1′-biphenyl]-4-yl)-N-(tert-butoxycarbonyl)-4,4,4-trifluorobutylsulfonimidoyl)-2-((tert-butoxycarbonyl)amino)butanoate (78 mg, crude) was obtained as colorless oil. ¹H NMR (400 MHz, CDCl₃-d) δ 7.68-7.55 (m, 4H), 7.47 (t, J=7.6 Hz, 2H), 7.42-7.33 (m, 3H), 5.27-5.10 (m, 1H), 4.21 (br d, J=3.5 Hz, 1H), 3.60-3.05 (m, 5H), 2.69-2.52 (m, 1H), 2.52-2.24 (m, 2H), 2.17-2.06 (m, 1H), 1.50-1.36 (m, 27H)

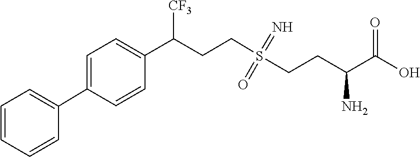 [SMILES: N=S(=O)(CCC(c1ccc(-c2ccccc2)cc1)C(F)(F)F)CC[C@H](N)C(=O)O]

A solution of (2S)-tert-butyl 4-(3-([1,1′-biphenyl]-4-yl)-N-(tert-butoxycarbonyl)-4,4,4-trifluorobutylsulfonimidoyl)-2-((tert-butoxycarbonyl)amino)butanoate (70 mg, 102 μmol) in HCl/dioxane (4 M, 10 mL). The mixture was stirred at 15° C. for 16 hr. The reaction mixture was concentrated under reduced pressure and the residue was purified by prep-HPLC (column: C18-1 150×30 mm×5 um; mobile phase: [water(FA)-ACN]; B %: 25%-55%, 10 min) to give (2S)-4-(3-([1,1′-biphenyl]-4-yl)-4,4,4-trifluorobutylsulfonimidoyl)-2-aminobutanoic acid (Compound 211) (26 mg, 53% yield, FA) as a white solid. LCMS: Rt=2.207 min, (ES⁺) m/z (M+H)⁺=429.0; HPLC Conditions C; ¹H NMR (400 MHz, D₂O) δ 7.66 (dd, J=8.1, 19.3 Hz, 4H), 7.52-7.41 (m, 4H), 7.39-7.32 (m, 1H), 3.85-3.61 (m, 2H), 3.43-3.33 (m, 1H), 3.28-3.08 (m, 2H), 2.99-2.81 (m, 1H), 2.65-2.39 (m, 2H), 2.36-2.20 (m, 2H).

### Exemplary Embodiment 1aa13

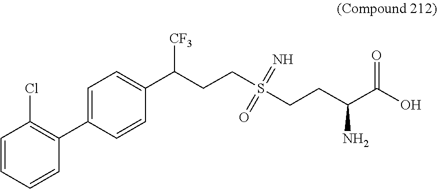 [SMILES: N=S(=O)(CCC(c1ccc(-c2ccccc2Cl)cc1)C(F)(F)F)CC[C@H](N)C(=O)O]

### (2S)-2-amino-4-(3-(2′-chloro-[1,1′-biphenyl]-4-yl)-4,4,4-trifluorobutylsulfonimidoyl)butanoic acid

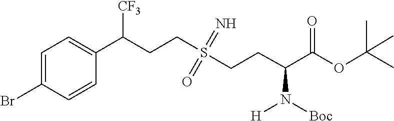 [SMILES: CC(C)(C)OC(=O)N[C@@H](CCS(=N)(=O)CCC(c1ccc(Br)cc1)C(F)(F)F)C(=O)OC(C)(C)C]

To a solution of (2S)-tert-butyl 4-((3-(4-bromophenyl)-4,4,4-trifluorobutyl)thio)-2-((tert-butoxycarbonyl)amino)butanoate (4.80 g, 8.63 mmol) in i-PrOH (50 mL) was added PhI(OAc)₂ (5.56 g, 17.5 mmol) and ammonium carbamate (3.37 g, 43.3 mmol). The mixture was stirred at 15° C. for 16 h and the reaction mixture was concentrated under reduced pressure. The mixture was poured into water (10 mL) and extracted with EtOAc (15 mL×3). The combined organic layers were dried over Na₂SO₄, filtered, and concentrated. The residue was purified by column chromatography (SiO₂, petroleum ether: ethyl acetate=54:46) to give (2S)-tert-butyl 4-(3-(4-bromophenyl)-4,4,4-trifluorobutylsulfonimidoyl)-2-((tert-butoxycarbonyl)amino)butanoate (4.08 g, 81% yield) as a colorless oil. ¹H NMR (400 MHz, CDCl₃-d) δ 7.54 (d, J=8.3 Hz, 2H), 7.20 (d, J=8.3 Hz, 2H), 5.21 (br s, 1H), 4.25 (br d, J=3.5 Hz, 1H), 3.62-3.44 (m, 1H), 3.19-3.07 (m, 1H), 3.05-2.77 (m, 3H), 2.67-2.54 (m, 1H), 2.43-2.29 (m, 2H), 2.09-2.01 (m, 1H), 1.46 (d, J=11.5 Hz, 18H).

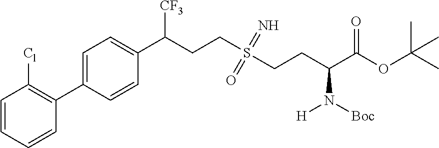 [SMILES: CC(C)(C)OC(=O)N[C@@H](CCS(=N)(=O)CCC(c1ccc(-c2ccccc2Cl)cc1)C(F)(F)F)C(=O)OC(C)(C)C]

To a solution of (2S)-tert-butyl 4-(3-(4-bromophenyl)-4,4,4-trifluorobutylsulfonimidoyl)-2-((tert-butoxycarbonyl)amino)butanoate (50.0 mg, 85.1 μmol) and (2-chlorophenyl)boronic acid (15.9 mg, 102 μmol) in dioxane (3 mL) and H₂O (0.6 mL) was added K₂CO₃ (35.2 mg, 255 μmol) and Pd(dppf)Cl₂ (3.11 mg, 4.26 μmol). The mixture was stirred at 100° C. for 5 h under N₂. The mixture was poured into water (5 mL) and extracted with EtOAc (5 mL×3). The combined organic layers were dried over Na₂SO₄, filtered, and concentrated under reduced pressure. The residue was purified by prep-TLC (SiO₂, Petroleum ether: ethyl acetate=1:1, R_{f}=0.43) to give (2S)-tert-butyl 2-((tert-butoxycarbonyl)amino)-4-(3-(2′-chloro-[1,1′-biphenyl]-4-yl)-4,4,4-trifluorobutylsulfonimidoyl)butanoate (50 mg, 95% yield) as a white solid. ¹H NMR (400 MHz, CDCl₃-d) δ 7.49 (d, J=7.6 Hz, 3H), 7.41-7.30 (m, 5H), 5.30-5.16 (m, 1H), 4.34-4.21 (m, 1H), 3.66-3.53 (m, 1H), 3.29-2.88 (m, 4H), 2.73-2.57 (m, 1H), 2.53-2.29 (m, 2H), 2.15-2.07 (m, 1H), 1.46 (d, J=12.7 Hz, 18H).

A solution of (2S)-tert-butyl 2-((tert-butoxycarbonyl)amino)-4-(3-(2′-chloro-[1,1′-biphenyl]-4-yl)-4,4,4-trifluorobutylsulfonimidoyl)butanoate (50 mg, 80.7 μmol) in HCl/dioxane (4 M, 5 mL) was stirred at 15° C. for 16 hr. The reaction mixture was concentrated and the residue was purified by prep-HPLC (column: Phenomenex Luna C1875×30 mm×3 um; mobile phase: [water(FA)-ACN]; B %: 1%-50%, 8 min) to give (2S)-2-amino-4-(3-(2′-chloro-[1,1′-biphenyl]-4-yl)-4,4,4-trifluorobutylsulfonimidoyl)butanoic acid (Compound 212) (20 mg, 49% yield, FA) as a white solid. LCMS: Rt=2.298 (ES⁺) m/z (M+H)⁺=463.0; HPLC Conditions C; ¹H NMR (400 MHz, METHANOL-d4) 6=7.63-7.45 (m, 5H), 7.43-7.28 (m, 3H), 3.88-3.65 (m, 2H), 3.51-3.36 (m, 2H), 3.23-3.10 (m, 1H), 3.04-2.87 (m, 1H), 2.68-2.42 (m, 2H), 2.40-2.23 (m, 2H).

### Exemplary Embodiment 1aa14

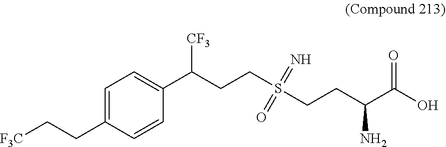 [SMILES: N=S(=O)(CCC(c1ccc(CCC(F)(F)F)cc1)C(F)(F)F)CC[C@H](N)C(=O)O]

### (2S)-2-amino-4-(4,4,4-trifluoro-3-(4-(3,3,3-trifluoropropyl)phenyl)butylsulfonimidoyl)butanoic acid

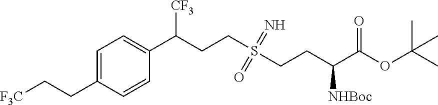 [SMILES: CC(C)(C)OC(=O)N[C@@H](CCS(=N)(=O)CCC(c1ccc(CCC(F)(F)F)cc1)C(F)(F)F)C(=O)OC(C)(C)C]

A mixture of tert-butyl (2S)-4-[[3-(4-bromophenyl)-4,4,4-trifluoro-butyl]sulfonimidoyl]-2-(tert-butoxycarbonylamino)butanoate (85.4 mg, 146 μmol, 1 eq), potassium 3,3,3-trifluoropropane-1-trifluoroborate (44.5 mg, 218 μmol, 1.5 eq), Cs₂CO₃ (142 mg, 437 μmol, 3 eq) and Pd(dppf)Cl₂ (7.13 mg, 8.73 μmol, 0.06 eq) in toluene (3 mL) and H₂O (0.3 mL) was degassed and purged 3 times with N₂, and then the mixture was stirred at 120° C. for 12 h under N₂ atmosphere. The reaction mixture was diluted with H₂O (3 mL) and extracted with EtOAc (5 mL×3). The combined organic phase was washed with brine (5 mL), dried with anhydrous Na₂SO₄, filtered, and concentrated under reduced pressure. The residue was purified by prep-TLC (DCM/MeOH=10:1) to give (2S)-tert-butyl 2-((tert-butoxycarbonyl)amino)-4-(4,4,4-trifluoro-3-(4-(3,3,3-trifluoropropyl)phenyl)butylsulfonimidoyl)butanoate (50 mg, 82.69 μmol, 56.86% yield) as a yellow oil. ¹H NMR (400 MHz, CDCl₃-d) δ 7.25 (d, 4H), 5.25-5.17 (m, 1H), 4.30-4.21 (m, 1H), 3.21-2.85 (m, 5H), 2.66-2.55 (m, 1H), 2.49-2.29 (m, 4H), 2.11-1.99 (m, 1H), 1.51-1.40 (m, 18H).

A solution of (2S)-tert-butyl 2-((tert-butoxycarbonyl)amino)-4-(4,4,4-trifluoro-3-(4-(3,3,3-trifluoropropyl)phenyl)butylsulfonimidoyl)butanoate (50 mg, 82.69 μmol, 1 eq) in HCl/dioxane (20 mL) was stirred at 20° C. for 12 h. The reaction mixture was concentrated under reduced pressure and the residue was purified by prep-HPLC (column: Waters Xbridge Prep OBD C18 150×40 mm×10 μm; mobile phase: [water (NH₄HCO₃)-MeCN]; B %: 10-60%, 8 min) to give (2S)-2-amino-4-(4,4,4-trifluoro-3-(4-(3,3,3-trifluoropropyl)phenyl)butylsulfonimidoyl)butanoic acid (18.78 mg, 41.04 μmol, 49.63% yield) as a white solid. LCMS: Rt=2.484 min., (ES⁺) m/z (M+H)⁺=449.2, HPLC Conditions: B. ¹H NMR (400 MHz, D₂O) δ 7.33 (s, 4H), 3.76 (q, 1H), 3.68-3.59 (m, 1H), 3.39-3.14 (m, 3H), 2.97-2.83 (m, 3H), 2.56-2.32 (m, 4H), 2.24-2.15 (m, 2H).

### Exemplary Embodiment 1aa15

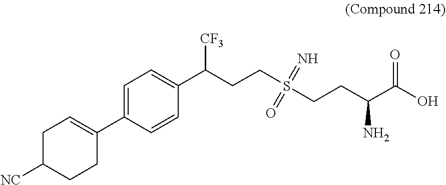 [SMILES: N#CC1CC=C(c2ccc(C(CCS(=N)(=O)CC[C@H](N)C(=O)O)C(F)(F)F)cc2)CC1]

### (2S)-2-amino-4-(3-(4′-cyano-2′,3′,4′,5′-tetrahydro-[1,1′-biphenyl]-4-yl)-4,4,4-trifluorobutylsulfonimidoyl)butanoic acid

A mixture of tert-butyl (2S)-4-[[3-(4-bromophenyl)-4,4,4-trifluoro-butyl]sulfonimidoyl]-2-(tert-butoxycarbonylamino)butanoate (300 mg, 511 μmol, 1 eq), 4-(4,4,5,5-tetramethyl-1,3,2-dioxaborolan-2-yl)cyclohex-3-ene-1-carbonitrile (179 mg, 766 μmol, 1.5 eq), Pd(dppf)Cl₂ (41.7 mg, 51.1 μmol, 0.1 eq) and K₂CO₃ (212 mg, 1.53 mmol, 3 eq) in dioxane (8 mL), H₂O (1.6 mL) was degassed and purged 3 times with N₂, and then the mixture was stirred at 100° C. for 12 h under N₂ atmosphere. The reaction mixture was diluted with water (10 mL) and extracted with EtOAc (30 mL×3). The combined organic layers were concentrated under reduced pressure and the residue was purified by column chromatography (SiO₂, petroleum ether/EtOAc=100:0 to 50:50) to give compound tert-butyl (2S)-2-((tert-butoxycarbonyl)amino)-4-(3-(4′-cyano-2′,3′,4′,5′-tetrahydro-[1,1′-biphenyl]-4-yl)-4,4,4-trifluorobutylsulfonimidoyl)butanoate (230 mg, 375 μmol, 73.4% yield) as a yellow solid. ¹H NMR (400 MHz, CDCl₃-d) δ 7.31 (d, 2H), 7.20 (br s, 1H), 7.18 (br s, 1H), 6.00 (br t, 1H), 5.12 (br s, 1H), 4.17 (br s, 1H), 3.48-3.37 (m, 1H), 3.11-2.99 (m, 1H), 2.98-2.68 (m, 4H), 2.64-2.39 (m, 5H), 2.37-2.20 (m, 2H), 2.16-2.06 (m, 1H), 2.05-1.93 (m, 2H), 1.47-1.30 (m, 18H).

To a solution of compound tert-butyl (2S)-2-((tert-butoxycarbonyl)amino)-4-(3-(4′-cyano-2′,3′,4′,5′-tetrahydro-[1,1′-biphenyl]-4-yl)-4,4,4-trifluorobutylsulfonimidoyl)butanoate (50.0 mg, 81.5 μmol, 1 eq) in DCM (2.5 mL) was added TFA (0.5 mL). The mixture was stirred at 18° C. for 12 h. The reaction mixture was concentrated under reduced pressure and the residue was purified by prep-HPLC (column: Phenomenex Luna C18 100×40 mm×3 μm; mobile phase: [water (FA)-MeCN]; B %: 1-50%, 8 min) to give (2S)-2-amino-4-(3-(4′-cyano-2′,3′,4′,5′-tetrahydro-[1,1′-biphenyl]-4-yl)-4,4,4-trifluorobutylsulfonimidoyl)butanoic acid (25.83 mg, 54.1 μmol, 66.3% yield) as a white solid. LCMS: Rt=1.977 min., (ES⁺) m/z (M+H)⁺=458.2, HPLC Conditions: C. ¹H NMR (400 MHz, MeOD) δ 8.17 (s, 1H), 7.52-7.44 (m, 2H), 7.37 (d, 2H), 6.15 (br s, 1H), 3.70 (td, 2H), 3.43-3.34 (m, 1H), 3.30-3.20 (m, 1H), 3.18-3.04 (m, 2H), 2.95-2.81 (m, 1H), 2.71-2.51 (m, 5H), 2.49-2.39 (m, 1H), 2.36-2.22 (m, 2H), 2.21-2.01 (m, 2H).

### Exemplary Embodiment 1aa16

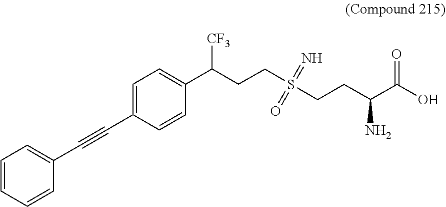 [SMILES: N=S(=O)(CCC(c1ccc(C#Cc2ccccc2)cc1)C(F)(F)F)CC[C@H](N)C(=O)O]

### (2S)-2-amino-4-(4,4,4-trifluoro-3-(4-(phenylethynyl)phenyl)butylsulfonimidoyl)butanoic acid

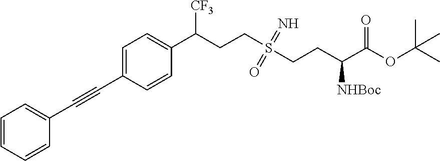 [SMILES: CC(C)(C)OC(=O)N[C@@H](CCS(=N)(=O)CCC(c1ccc(C#Cc2ccccc2)cc1)C(F)(F)F)C(=O)OC(C)(C)C]

To a solution of tert-butyl (2S)-4-[[3-(4-bromophenyl)-4,4,4-trifluoro-butyl]sulfonimidoyl]-2-(tert-butoxycarbonylamino)butanoate (150 mg, 255 μmol, 1 eq), CuI (14.5 mg, 76.6 μmol, 0.3 eq) and Pd(PPh₃)₄ (29.5 mg, 25.5 μmol, 0.1 eq) in TEA (2.5 mL) was added ethynylbenzene (78.2 mg, 765 μmol, 84.1 uL, 3 eq). The mixture was stirred at 130° C. for 16 h. The reaction mixture was poured into H₂O (20 mL) and the aqueous phase was extracted with EtOAc (20 mL×2). The combined organic phase was washed with brine (20 mL×2), dried with anhydrous Na₂SO₄, filtered, and concentrated in vacuo. The residue was purified by prep-TLC (petroleum ether:EtOAc=1:1, R_{f}=0.5) to give tert-butyl (2S)-2-((tert-butoxycarbonyl)amino)-4-(4,4,4-trifluoro-3-(4-(phenylethynyl)phenyl)butylsulfonimidoyl)butanoate (100 mg, 164 μmol, 64.3% yield) as colorless oil. ¹H NMR (400 MHz, CDCl₃-d) δ 7.59-7.51 (m, 4H), 7.40-7.34 (m, 3H), 7.31 (d, 2H), 5.21 (br s, 1H), 4.32-4.17 (m, 1H), 3.61-3.50 (m, 1H), 3.17-3.08 (m, 1H), 3.04-2.80 (m, 3H), 2.66-2.57 (m, 1H), 2.45-2.30 (m, 2H), 2.13-2.01 (m, 1H), 1.46 (d, 18H).

A solution of tert-butyl (2S)-2-((tert-butoxycarbonyl)amino)-4-(4,4,4-trifluoro-3-(4-(phenylethynyl)phenyl)butylsulfonimidoyl)butanoate (100 mg, 164 μmol, 1 eq) in HCl/dioxane (4 mL) was stirred at 20° C. for 16 h. The reaction mixture was concentrated and the residue was purified by prep-HPLC (column: Phenomenex C18 (75×30 mm, 3 μm); mobile phase: [water (FA)-MeCN]) to give (2S)-2-amino-4-(4,4,4-trifluoro-3-(4-(phenylethynyl)phenyl)butylsulfonimidoyl)butanoic acid (19.2 mg, 41.3 μmol, 25.1% yield) as white solid. LCMS: Rt=2.292 min, (ES⁺) m/z (M+H)⁺=453.1, HPLC Conditions: C. ¹H NMR (400 MHz, DMSO-d₆) δ 7.62 (d, 2H), 7.58-7.55 (m, 2H), 7.48-7.40 (m, 5H), 4.11-3.84 (m, 2H), 3.28-3.22 (m, 1H), 3.09 (ddd, 1H), 3.02-2.93 (m, 1H), 2.69-2.56 (m, 1H), 2.40-2.27 (m, 2H), 2.11-2.00 (m, 1H), 1.99-1.89 (m, 1H).

### Exemplary Embodiment 1aa17

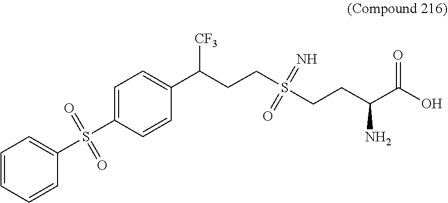 [SMILES: N=S(=O)(CCC(c1ccc(S(=O)(=O)c2ccccc2)cc1)C(F)(F)F)CC[C@H](N)C(=O)O]

### (2S)-2-amino-4-(4, 4,4-trifluoro-3-(4-(phenylsulfonyl) phenyl) butylsulfonimidoyl) butanoic acid

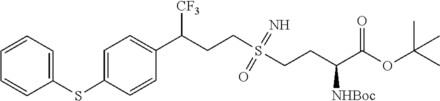 [SMILES: CC(C)(C)OC(=O)N[C@@H](CCS(=N)(=O)CCC(c1ccc(Sc2ccccc2)cc1)C(F)(F)F)C(=O)OC(C)(C)C]

A mixture of tert-butyl (2S)-4-(3-(4-bromophenyl)-4,4,4-trifluorobutylsulfonimidoyl)-2-((tert-butoxycarbonyl)amino)butanoate (150 mg, 255 μmol, 1 eq), benzenethiol (56.3 mg, 511 μmol, 52.2 μL, 2 eq), DIEA (99.0 mg, 766 μmol, 133 μL, 3 eq), Pd₂(dba)₃ (70.1 mg, 76.6 μmol, 0.3 eq) and Xantphos (133 mg, 230 μmol, 0.9 eq) in dioxane (2 mL) was degassed and purged 3 times with N₂, and then the mixture was stirred at 110° C. for 6 h under N₂ atmosphere. The mixture was poured into H₂O (40 mL) and extracted with EtOAc (40 mL×3). The combined organic layers were dried over Na₂SO₄, filtered, and concentrated under reduced pressure. The residue was purified by prep-TLC (SiO₂, DCM/MeOH=10:1) to obtain tert-butyl (2S)-2-((tert-butoxycarbonyl) amino)-4-(4, 4, 4-trifluoro-3-(4-(phenylthio) phenyl) butylsulfonimidoyl) butanoate (117 mg, 190 μmol, 74.3% yield) as a yellow oil.

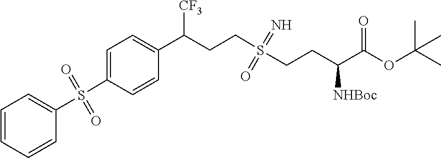 [SMILES: CC(C)(C)OC(=O)N[C@@H](CCS(=N)(=O)CCC(c1ccc(S(=O)(=O)c2ccccc2)cc1)C(F)(F)F)C(=O)OC(C)(C)C]

To a solution of (2S)-2-((tert-butoxycarbonyl) amino)-4-(4, 4, 4-trifluoro-3-(4-(phenylthio) phenyl) butylsulfonimidoyl) butanoate (97 mg, 157 μmol, 1 eq) in DCM (2 mL) was added m-CPBA (63.9 mg, 315 μmol, 85% purity, 2 eq) at 0° C. The mixture was stirred at 20° C. for 1 h. The mixture was poured into H₂O (40 mL) and extracted with EtOAc (40 mL×3). The combined organic layers were dried over Na₂SO₄, filtered, and concentrated under reduced pressure. The residue was purified by prep-TLC (SiO₂, DCM:MeOH=10:1) to give tert-butyl (2S)-2-((tert-butoxycarbonyl) amino)-4-(4, 4, 4-trifluoro-3-(4-(phenylsulfonyl) phenyl) butylsulfonimidoyl) butanoate (64.8 mg, 99.9 μmol, 63.5% yield) as a colorless oil. ¹H NMR (400 MHz, CDCl₃-d) δ 7.98 (br d, 5H), 7.54 (br d, 4H), 5.51-5.38 (m, 1H), 4.38-4.19 (m, 1H), 3.91-3.72 (m, 3H), 3.62-3.35 (m, 2H), 2.60-2.35 (m, 2H), 2.32-2.12 (m, 1H), 1.49 (s, 9H), 1.44 (d, 9H).

A solution of tert-butyl (2S)-2-((tert-butoxycarbonyl) amino)-4-(4, 4, 4-trifluoro-3-(4-(phenylsulfonyl) phenyl) butylsulfonimidoyl) butanoate (64.8 mg, 99.9 μmol, 1 eq) in HCl/dioxane (5 M, 10 mL) was stirred at 25° C. for 6 h. The mixture was concentrated and the residue was purified by prep-HPLC: (column: Waters Xbridge C₁₈ (150×50 mm, 10 μm); mobile phase: [H₂O (10 mM NH₄HCO₃)-MeCN]; gradient: 25-55% B over 8.0 min) to give (2S)-2-amino-4-(4, 4, 4-trifluoro-3-(4-(phenylsulfonyl) phenyl) butylsulfonimidoyl) butanoic acid (3.34 mg, 6.78 μmol, 6.79% yield) as a white solid. LCMS: Rt=2.022 min, (ES⁺) m/z (M+H)⁺=493.1, HPLC Conditions: C. ¹H NMR (400 MHz, MeOD-d₄) δ 8.04-7.96 (m, 4H), 7.70-7.57 (m, 5H), 3.96-3.81 (m, 1H), 3.68-3.56 (m, 1H), 3.29-3.16 (m, 2H), 3.14-3.03 (m, 1H), 2.92-2.74 (m, 1H), 2.65-2.51 (m, 1H), 2.50-2.34 (m, 1H), 2.31-2.17 (m, 2H).

The compounds described in Table 11 were prepared using the general methods outlined for exemplary embodiment 1aa13.

TABLE 11
Characterization of Compounds 217-291

| | | | HPLC | |
|---|---|---|---|---|
| Compound | | MS | Rt | HPLC |
| # | Structure | (M + H)⁺ | (min) | cond. |
| 217 | 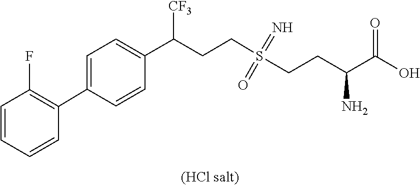 [SMILES: N=S(=O)(CCC(c1ccc(-c2ccccc2F)cc1)C(F)(F)F)CC[C@H](N)C(=O)O] | 447.1 | 2.17 | C |
| | ¹H NMR (400 MHz, MeOD-d4) δ 7.61 (br d, 2H), | | | |
| | 7.54-7.46 (m, 3H), 7.43-7.35 (m, 1H), 7.31-7.16 | | | |
| | (m, 2H), 3.84-3.72 (m, 1H), 3.72-3.63 (m, 1H), | | | |
| | 3.30-3.24 (m, 2H), 3.22-3.10 (m, 1H), 3.02-2.84 | | | |
| | (m, 1H), 2.67-2.42 (m, 2H), 2.38-2.22 (m, 2H). | | | |
| 218 | 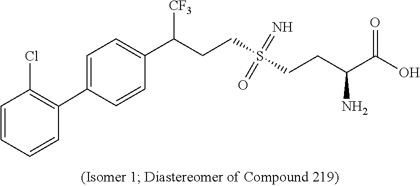 | 463.1 | 2.147 | C |
| | ¹H NMR (400 MHz, MeOD-d₄) δ 7.52-7.46 (m, | | | |
| | 5H), 7.41-7.32 (m, 3H), 3.89-3.83 (m, 1H), 3.83- | | | |
| | 3.72 (m, 1H), 3.41-3.33 (m, 1H), 3.26-3.10 (m, | | | |
| | 2H), 3.01-2.94 (m, 1H), 2.63-2.55 (m, 1H), 2.52- | | | |
| | 2.44 (m, 1H), 2.36-2.27 (m, 2H) | | | |
| 219 | 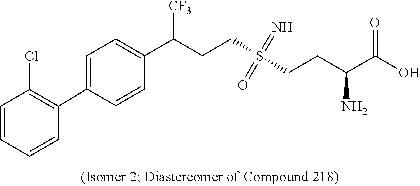 | 463.1 | 2.148 | C |
| | ¹H NMR (400 MHz, MeOD-d₄) δ 7.55-7.42 (m, | | | |
| | 5H), 7.41-7.29 (m, 3H), 3.88-3.74 (m, 2H), 3.45- | | | |
| | 3.36 (m, 1H), 3.28-3.24 (m, 1H), 3.22-3.14 (m, | | | |
| | 1H), 3.04-2.92 (m, 1H), 2.67-2.55 (m, 1H), 2.54- | | | |
| | 2.42 (m, 1H), 2.41-2.23 (m, 2H) | | | |
| 220 | 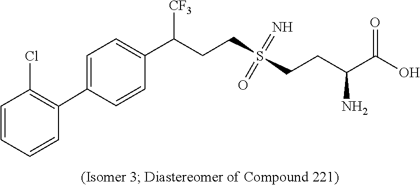 | 463.1 | 2.143 | C |
| | ¹H NMR (400 MHz, MeOD-d₄) δ 7.55-7.42 (m, | | | |
| | 5H), 7.41-7.29 (m, 3H), 3.88-3.74 (m, 2H), 3.45- | | | |
| | 3.36 (m, 1H), 3.28-3.24 (m, 1H), 3.22-3.14 (m, | | | |
| | 1H), 3.04-2.92 (m, 1H), 2.67-2.55 (m, 1H), 2.54- | | | |
| | 2.42 (m, 1H), 2.41-2.23 (m, 2H) | | | |
| 221 | 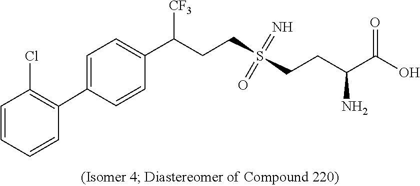 | 463.1 | 2.144 | C |
| | ¹H NMR (400 MHz, MeOD-d₄) δ 7.53-7.44 (m, | | | |
| | 5H), 7.40-7.31 (m, 3H), 3.84-3.74 (m, 1H), 3.71 | | | |
| | (t, 1H), 3.45-3.34 (m, 1H), 3.28-3.14 (m, 2H), | | | |
| | 2.98-2.87 (m, 1H), 2.69-2.43 (m, 2H), 2.38-2.21 | | | |
| | (m, 2H). | | | |
| 222 | 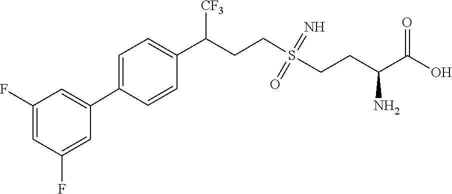 [SMILES: N=S(=O)(CCC(c1ccc(-c2cc(F)cc(F)c2)cc1)C(F)(F)F)CC[C@H](N)C(=O)O] | 465.2 | 2.69 | B |
| | ¹H NMR (400 MHz, MeOD-d₄) δ 7.71 (d, 2H), 7.51 | | | |
| | (d, 2H), 7.31-7.24 (m, 2H), 6.95 (tt, 1H), 3.85- | | | |
| | 3.62 (m, 2H), 3.42-3.34 (m, 1H), 3.27-3.22 (m, | | | |
| | 1H), 3.21-3.09 (m, 1H), 2.96-2.84 (m, 1H), 2.65- | | | |
| | 2.41 (m, 2H), 2.34-2.21 (m, 2H). | | | |
| 223 | 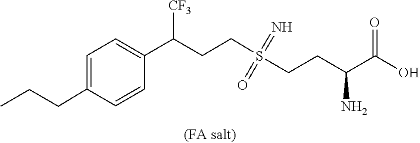 [SMILES: CCCc1ccc(C(CCS(=N)(=O)CC[C@H](N)C(=O)O)C(F)(F)F)cc1] | 395.1 | 2.213 | C |
| | ¹H NMR (400 MHz, D₂O) δ 7.49-7.17 (m, 4H), | | | |
| | 3.90-3.76 (m, 1H), 3.74-3.57 (m, 1H), 3.47-3.15 | | | |
| | (m, 3H), 3.06-2.88 (m, 1H), 2.66-2.34 (m, 4H), | | | |
| | 2.32-2.14 (m, 2H), 1.60 (sxt, 2H), 0.88 (t, 3H) | | | |
| 224 | 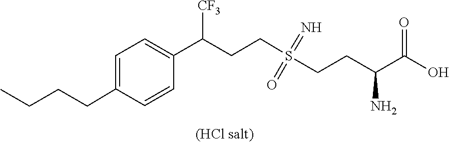 [SMILES: CCCCc1ccc(C(CCS(=N)(=O)CC[C@H](N)C(=O)O)C(F)(F)F)cc1] | 409.1 | 2.22 | C |
| | ¹H NMR (400 MHz, D₂O) δ 7.37-7.30 (m, 4H), | | | |
| | 3.81 (q, 1H), 3.66 (br s, 1H), 3.43-3.19 (m, 3H), | | | |
| | 3.05-2.92 (m, 1H), 2.66-2.60 (m, 2H), 2.59-2.48 | | | |
| | (m, 1H), 2.47-2.32 (m, 1H), 2.30-2.20 (m, 2H), | | | |
| | 1.56 (quin, 2H), 1.30 (sxt, 2H), 0.87 (t, 3H). | | | |
| 225 | 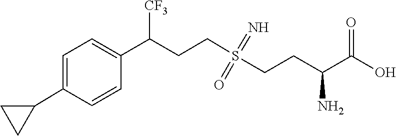 [SMILES: N=S(=O)(CCC(c1ccc(C2CC2)cc1)C(F)(F)F)CC[C@H](N)C(=O)O] | 393.2 | 2.33 | B |
| | ¹H NMR (400 MHz, D₂O) δ 7.25 (d, 2H), 7.12 (d, | | | |
| | 2H), 3.77-3.70 (m, 1H), 3.63-3.52 (m, 1H), 3.36- | | | |
| | 3.26 (m, 1H), 3.21-3.10 (m, 2H), 2.95-2.80 (m, | | | |
| | 1H), 2.55-2.27 (m, 2H), 2.24-2.14 (m, 2H), 1.93- | | | |
| | 1.83 (m, 1H), 0.98-0.87 (m, 2H), 0.66-0.58 (m, | | | |
| | 2H) | | | |
| 226 | 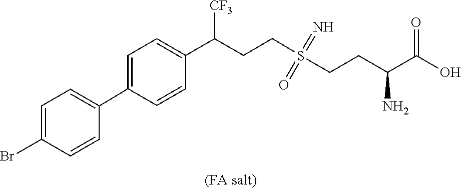 [SMILES: N=S(=O)(CCC(c1ccc(-c2ccc(Br)cc2)cc1)C(F)(F)F)CC[C@H](N)C(=O)O] | 507.1 | 2.67 | B |
| | ¹H NMR (400 MHz, MeOD-d₄) δ 8.49 (s, 1H), 7.68 | | | |
| | (d, 2H), 7.64-7.55 (m, 4H), 7.49 (d, 2H), 3.86- | | | |
| | 3.61 (m, 2H), 3.36 (br s, 1H), 3.26 (br dd, 8.6 Hz, | | | |
| | 1H), 3.18-3.09 (m, 1H), 2.99-2.81 (m, 1H), 2.69- | | | |
| | 2.40 (m, 2H), 2.36-2.17 (m, 2H) | | | |
| 227 | 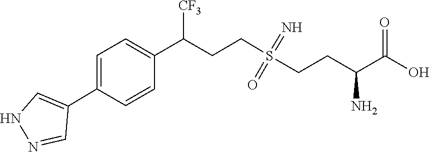 [SMILES: N=S(=O)(CCC(c1ccc(-c2cn[nH]c2)cc1)C(F)(F)F)CC[C@H](N)C(=O)O] | 419.2 | 2.04 | B |
| | ¹H NMR (400 MHz, D₂O) δ 8.05 (s, 2H), 7.68 (d, | | | |
| | 2H), 7.45 (br d, 2H), 3.86-3.66 (m, 2H), 3.46- | | | |
| | 3.19 (m, 3H), 3.08-2.94 (m, 1H), 2.62-2.38 (m, | | | |
| | 2H), 2.30-2.19 (m, 2H) | | | |
| 228 | 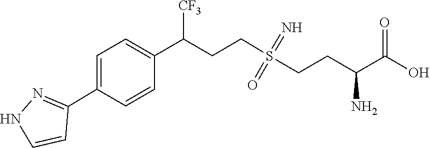 [SMILES: N=S(=O)(CCC(c1ccc(-c2cc[nH]n2)cc1)C(F)(F)F)CC[C@H](N)C(=O)O] | 419.2 | 2.11 | B |
| | ¹H NMR (400 MHz, D₂O) δ 7.80 (br d, 2H), 7.72 | | | |
| | (br d, 1H), 7.47 (br d, 2H), 6.73 (d, 1H), 3.82-3.65 | | | |
| | (m, 2H), 3.40-3.16 (m, 3H), 3.04-2.93 (m, 1H), | | | |
| | 2.59-2.36 (m, 2H), 2.27-2.16 (m, 2H) | | | |
| 229 | 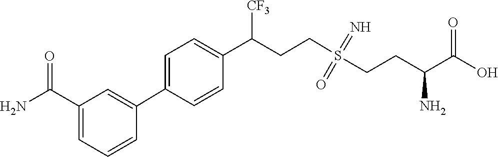 [SMILES: N=S(=O)(CCC(c1ccc(-c2cccc(C(N)=O)c2)cc1)C(F)(F)F)CC[C@H](N)C(=O)O] | 472.1 | 1.87 | C |
| | ¹H NMR (400 MHz, D₂O) δ 7.97 (s, 1H), 7.81 (br | | | |
| | d, 1H), 7.74 (br d, 1H), 7.69 (d, 2H), 7.54 (t, 1H), | | | |
| | 7.49 (br d, 2H), 3.81-3.67 (m, 2H), 3.41-3.30 (m, | | | |
| | 1H), 3.29-3.17 (m, 2H), 3.04-2.93 (m, 1H), 2.61- | | | |
| | 2.49 (m, 1H), 2.47-2.34 (m, 1H), 2.28-2.17 (m, | | | |
| | 2H). | | | |
| 230 | 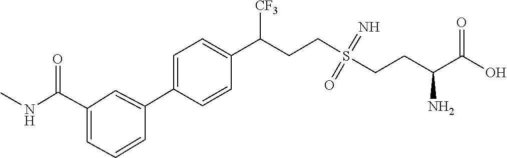 [SMILES: CNC(=O)c1cccc(-c2ccc(C(CCS(=N)(=O)CC[C@H](N)C(=O)O)C(F)(F)F)cc2)c1] | 486.2 | 2.00 | C |
| | ¹H NMR (400 MHz, D₂O) δ 7.94 (s, 1H), 7.83 (d, | | | |
| | 1H), 7.77-7.68 (m, 3H), 7.62-7.50 (m, 3H), 3.85- | | | |
| | 3.72 (m, 2H), 3.48-3.21 (m, 3H), 3.08-2.96 (m, | | | |
| | 1H), 2.92 (s, 3H), 2.65-2.39 (m, 2H), 2.33-2.20 | | | |
| | (m, 2H) | | | |
| 231 | 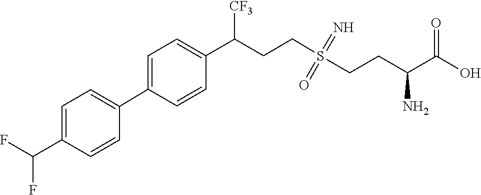 [SMILES: N=S(=O)(CCC(c1ccc(-c2ccc(C(F)F)cc2)cc1)C(F)(F)F)CC[C@H](N)C(=O)O] | 479.1 | 2.23 | C |
| | ¹H NMR (400 MHz, MeOD-d₄) δ 7.77 (dd, 4H), | | | |
| | 7.65 (d, 2H), 7.53 (d, 2H), 7.00-6.67 (m, 1H), 3.88- | | | |
| | 3.75 (m, 1H), 3.73-3.63 (m, 1H), 3.46-3.35 (m, | | | |
| | 1H), 3.31-3.11 (m, 2H), 3.00-2.84 (m, 1H), 2.70- | | | |
| | 2.43 (m, 2H), 2.37-2.24 (m, 2H). | | | |
| 232 | 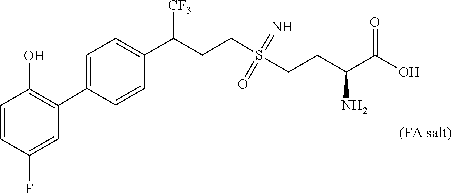 [SMILES: N=S(=O)(CCC(c1ccc(-c2cc(F)ccc2O)cc1)C(F)(F)F)CC[C@H](N)C(=O)O] | 463.1 | 2.08 | C |
| | ¹H NMR (400 MHz, MeOD-d₄) δ 7.64 (d, 2H), 7.43 | | | |
| | (d, 2H), 7.03 (dd, 1H), 6.95-6.80 (m, 2H), 3.80- | | | |
| | 3.63 (m, 2H), 3.41-3.34 (m, 1H), 3.30-3.24 (m, | | | |
| | 1H), 3.24-3.09 (m, 1H), 3.00-2.85 (m, 1H), 2.65- | | | |
| | 2.54 (m, 1H), 2.54-2.40 (m, 1H), 2.38-2.21 (m, | | | |
| | 2H) | | | |
| 233 | 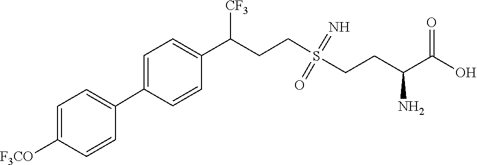 [SMILES: N=S(=O)(CCC(c1ccc(-c2ccc(OC(F)(F)F)cc2)cc1)C(F)(F)F)CC[C@H](N)C(=O)O] | 513.1 | 2.45 | C |
| | ¹H NMR (400 MHz, MeOD-d₄) δ 7.78-7.66 (m, | | | |
| | 4H), 7.50 (br d2H), 7.37 (br d, 2H), 3.86-3.72 (m, | | | |
| | 1H), 3.72-3.61 (m, 1H), 3.28-3.09 (m, 3H), 2.99- | | | |
| | 2.81 (m, 1H), 2.68-2.39 (m, 2H), 2.27-2.27 (m, | | | |
| | 1H), 2.36-2.18 (m, 1H) | | | |
| 234 | 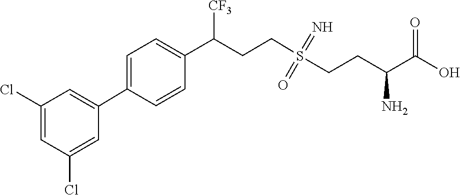 [SMILES: N=S(=O)(CCC(c1ccc(-c2cc(Cl)cc(Cl)c2)cc1)C(F)(F)F)CC[C@H](N)C(=O)O] | 497.0 | 2.41 | C |
| | ¹H NMR (400 MHz, MeOD-d₄) δ 7.70 (d, 2H), 7.64- | | | |
| | 7.61 (m, 2H), 7.52 (d, 2H), 7.45 (t, 1H), 3.85- | | | |
| | 3.63 (m, 2H), 3.45-3.32 (m, 1H), 3.24 (br d, 1H), | | | |
| | 3.19-3.09 (m, 1H), 2.98-2.81 (m, 1H), 2.67-2.54 | | | |
| | (m, 1H), 2.53-2.39 (m, 1H), 2.36-2.22 (m, 2H) | | | |
| 235 | 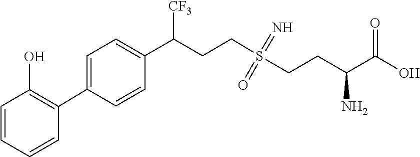 [SMILES: N=S(=O)(CCC(c1ccc(-c2ccccc2O)cc1)C(F)(F)F)CC[C@H](N)C(=O)O] | 445.2 | 2.15 | C |
| | ¹H NMR (400 MHz, MeOD-d₄) δ 6.07 (d, 2H), 5.86 | | | |
| | (d, 2H), 5.76-5.68 (m, 1H), 5.61 (dt, 1H), 5.42- | | | |
| | 5.28 (m, 2H), 2.24-2.05 (m, 2H), 1.87-1.78 (m, | | | |
| | 1H), 1.75-1.67 (m, 1H), 1.66-1.51 (m, 1H), 1.48- | | | |
| | 1.30 (m, 1H), 1.09-0.98 (m, 1H), 0.98-0.84 (m, | | | |
| | 1H), 0.82-0.66 (m, 2H) | | | |
| 236 | 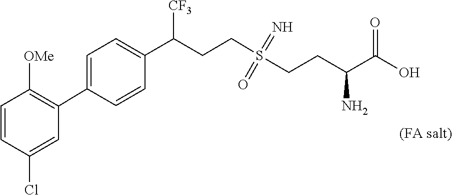 [SMILES: COc1ccc(Cl)cc1-c1ccc(C(CCS(=N)(=O)CC[C@H](N)C(=O)O)C(F)(F)F)cc1] | 493.1 | 2.28 | C |
| | ¹H NMR (400 MHz, MeOD) δ 7.47 (d, 2H), 7.36 | | | |
| | (d, 2H), 7.28-7.20 (m, 2H), 7.00 (d, 1H), 3.72- | | | |
| | 3.66 (m, 1H), 3.65-3.55 (m, 1H), 3.38-3.27 (m, | | | |
| | 1H), 3.21 (br s, 1H), 3.09 (td, 1H), 2.94-2.76 (m, | | | |
| | 1H), 2.60-2.46 (m, 1H), 2.46-2.35 (m, 1H), 2.30- | | | |
| | 2.12 (m, 2H) | | | |
| 237 | 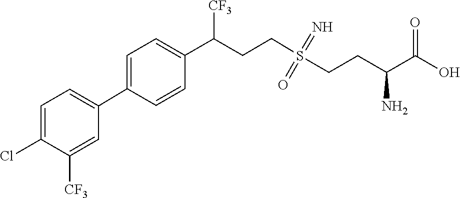 [SMILES: N=S(=O)(CCC(c1ccc(-c2ccc(Cl)c(C(F)(F)F)c2)cc1)C(F)(F)F)CC[C@H](N)C(=O)O] | 531.1 | 2.81 | B |
| | ¹H NMR (400 MHz, MeOH-d₄) δ 8.00 (s, 1H), 7.90 | | | |
| | (dd, 1H), 7.76-7.69 (m, 3H), 7.54 (d, 2H), 3.87- | | | |
| | 3.73 (m, 1H), 3.69-3.60 (m, 1H), 3.41-3.32 (m, | | | |
| | 1H), 3.28-3.21 (m, 1H), 3.19-3.09 (m, 1H), 2.97- | | | |
| | 2.82 (m, 1H), 2.67-2.42 (m, 2H), 2.32-2.21 (m, | | | |
| | 2H) | | | |
| 238 | 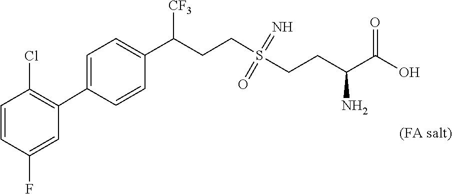 [SMILES: N=S(=O)(CCC(c1ccc(-c2cc(F)ccc2Cl)cc1)C(F)(F)F)CC[C@H](N)C(=O)O] | 481.0 | 2.23 | C |
| | ¹H NMR (400 MHz, MeOD-d₄) δ 8.17 (s, 1H), 7.59- | | | |
| | 7.46 (m, 5H), 7.25-7.09 (m, 2H), 3.87-3.76 (m, | | | |
| | 1H), 3.75-3.66 (m, 1H), 3.47-3.35 (m, 1H), 3.31- | | | |
| | 3.13 (m, 2H), 3.03-2.89 (m, 1H), 2.70-2.45 (m, | | | |
| | 2H), 2.40-2.24 (m, 2H) | | | |
| 239 | 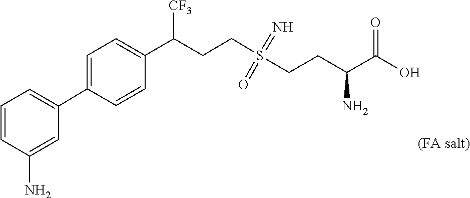 [SMILES: N=S(=O)(CCC(c1ccc(-c2cccc(N)c2)cc1)C(F)(F)F)CC[C@H](N)C(=O)O] | 444.1 | 1.68 | C |
| | ¹H NMR (400 MHz, D₂O) δ 8.36 (s, 1H), 7.65 (d, | | | |
| | 2H), 7.46 (br d, 2H), 7.32-7.22 (m, 1H), 7.13- | | | |
| | 7.02 (m, 2H), 6.82 (br d, 1H), 3.81-3.62 (m, 2H), | | | |
| | 3.43-3.15 (m, 3H), 3.05-2.87 (m, 1H), 2.60-2.33 | | | |
| | (m, 2H), 2.28-2.13 (m, 2H) | | | |
| 240 | 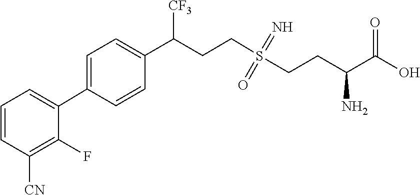 [SMILES: N#Cc1cccc(-c2ccc(C(CCS(=N)(=O)CC[C@H](N)C(=O)O)C(F)(F)F)cc2)c1F] | 472.1 | 2.11 | C |
| | ¹H NMR (400 MHz, MeOD-d₄) δ = 7.86 (br t, 1H), | | | |
| | 7.77 (br t, 1H), 7.70-7.60 (m, 2H), 7.55 (br d, 2H), | | | |
| | 7.46 (br t, 1H), 3.93-3.74 (m, 1H), 3.73-3.61 (m, | | | |
| | 1H), 3.59-3.36 (m, 2H), 3.21-3.04 (m, 1H), 3.01- | | | |
| | 2.77 (m, 1H), 2.71-2.38 (m, 2H), 2.29 (br d, 2H) | | | |
| 241 | 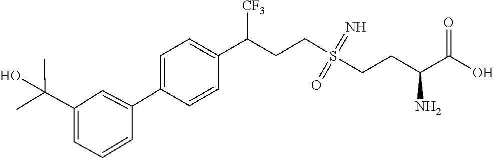 [SMILES: CC(C)(O)c1cccc(-c2ccc(C(CCS(=N)(=O)CC[C@H](N)C(=O)O)C(F)(F)F)cc2)c1] | 487.0 | 2.33 | B |
| | ¹H NMR (400 MHz, MeOD-d₄) δ 7.77 (s, 1H), 7.69 | | | |
| | (d, 2H), 7.54-7.45 (m, 4H), 7.44-7.37 (m, 1H), | | | |
| | 3.83-3.71 (m, 1H), 3.71-3.63 (m, 1H), 3.43-3.34 | | | |
| | (m, 1H), 3.29-3.23 (m, 1H), 3.16 (td, 1H), 3.02- | | | |
| | 2.80 (m, 1H), 2.67-2.55 (m, 1H), 2.54-2.38 (m, | | | |
| | 1H), 2.38-2.15 (m, 2H), 1.58 (s, 6H). | | | |
| 242 | 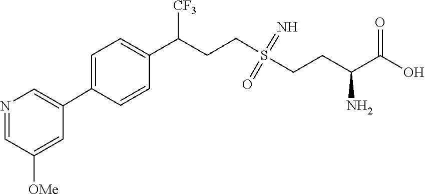 [SMILES: COc1cncc(-c2ccc(C(CCS(=N)(=O)CC[C@H](N)C(=O)O)C(F)(F)F)cc2)c1] | 460.2 | 1.77 | C |
| | ¹H NMR (400 MHz, D₂O) δ 8.45-8.36 (m, 1H), | | | |
| | 8.23 (d, 1H), 7.79-7.65 (m, 3H), 7.56 (d, 2H), 3.93 | | | |
| | (s, 3H), 3.87-3.72 (m, 2H), 3.46-3.33 (m, 1H), | | | |
| | 3.33-3.22 (m, 2H), 3.09-2.94 (m, 1H), 2.66-2.53 | | | |
| | (m, 1H), 2.52-2.40 (m, 1H), 2.35-2.14 (m, 2H) | | | |
| 243 | 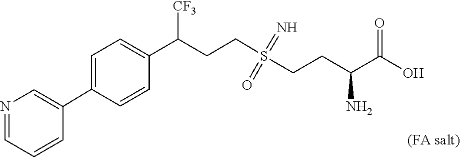 [SMILES: N=S(=O)(CCC(c1ccc(-c2cccnc2)cc1)C(F)(F)F)CC[C@H](N)C(=O)O] | 430.1 | 1.52 | C |
| | ¹H NMR (400 MHz, D₂O) δ 8.75 (d, 1H), 8.56- | | | |
| | 8.45 (m, 1H), 8.11 (br d, 1H), 7.70 (d, 2H), 7.61- | | | |
| | 7.50 (m, 3H), 3.85-3.71 (m, 2H), 3.44-3.23 (m, | | | |
| | 3H), 3.07-2.92 (m, 1H), 2.66-2.54 (m, 1H), 2.52- | | | |
| | 2.38 (m, 1H), 2.33-2.21 (m, 2H) | | | |
| 244 | 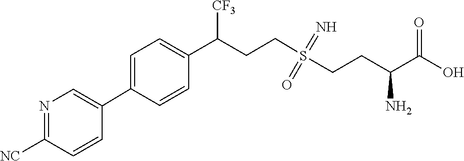 [SMILES: N#Cc1ccc(-c2ccc(C(CCS(=N)(=O)CC[C@H](N)C(=O)O)C(F)(F)F)cc2)cn1] | 455.1 | 1.98 | C |
| | ¹H NMR (400 MHz, D₂O) δ 8.96 (d, 1H), 8.27 (dd, | | | |
| | 1H), 8.00 (d, 1H), 7.80 (d, 2H), 7.61 (br d, 2H), | | | |
| | 3.81 (q, 2H), 3.43-3.23 (m, 3H), 3.07-2.96 (m, | | | |
| | 1H), 2.65-2.43 (m, 2H), 2.31-2.21 (m, 2H). | | | |
| 245 | 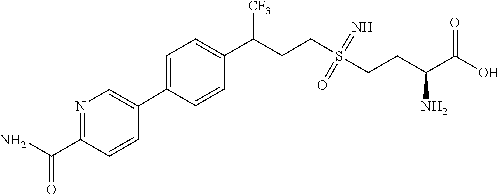 [SMILES: N=S(=O)(CCC(c1ccc(-c2ccc(C(N)=O)nc2)cc1)C(F)(F)F)CC[C@H](N)C(=O)O] | 473.1 | 1.81 | C |
| | ¹H NMR (400 MHz, DMSO-d6) δ 8.97 (d1H), 8.30 | | | |
| | (dd, 1H), 8.18-8.09 (m, 2H), 7.90-7.85 (m, 2H), | | | |
| | 7.68 (br s, 1H), 7.58-7.55 (m, 2H), 4.09-3.93 (m, | | | |
| | 2H), 3.16-2.92 (m, 3H), 2.71-2.58 (m, 1H), 2.45- | | | |
| | 2.31 (m, 2H), 2.13-1.88 (m, 2H). | | | |
| 246 | 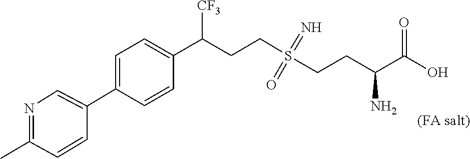 [SMILES: Cc1ccc(-c2ccc(C(CCS(=N)(=O)CC[C@H](N)C(=O)O)C(F)(F)F)cc2)cn1] | 444.1 | 1.60 | C |
| | ¹H NMR (400 MHz, D₂O) δ 8.64 (s, 1H), 8.04 (br | | | |
| | d, 1H), 7.71 (br d, 2H), 7.55 (br d, 2H), 7.43 (d, | | | |
| | 1H), 3.88-3.68 (m, 2H), 3.48-3.21 (m, 3H), 3.08- | | | |
| | 2.95 (m, 1H), 2.64-2.53 (m, 4H), 2.51-2.39 (m, | | | |
| | 1H), 2.33-2.18 (m, 2H) | | | |
| 247 | 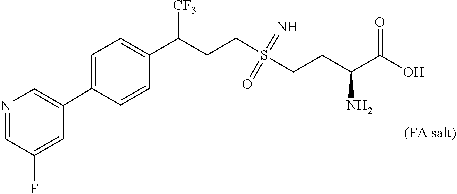 [SMILES: N=S(=O)(CCC(c1ccc(-c2cncc(F)c2)cc1)C(F)(F)F)CC[C@H](N)C(=O)O] | 448.1 | 1.91 | C |
| | ¹H NMR (400 MHz, MeOD-d₄) δ 8.74 (s, 1H), 8.50 | | | |
| | (d, 1H), 7.98 (td, 1H), 7.80 (d, 2H), 7.59 (d, 2H), | | | |
| | 3.90-3.77 (m, 1H), 3.75-3.65 (m, 1H), 3.47-3.35 | | | |
| | (m, 1H), 3.31-3.12 (m, 2H), 2.99-2.85 (m, 1H), | | | |
| | 2.69-2.43 (m, 2H), 2.38-2.22 (m, 2H) | | | |
| 248 | 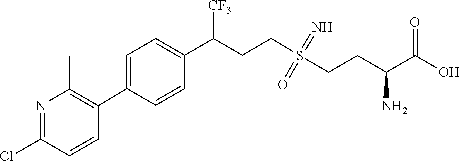 [SMILES: Cc1nc(Cl)ccc1-c1ccc(C(CCS(=N)(=O)CC[C@H](N)C(=O)O)C(F)(F)F)cc1] | 478.1 | 2.10 | C |
| | ¹H NMR (400 MHz, MeOD-d4) δ 7.67 (d, 1H), | | | |
| | 7.55-7.51 (m, 2H), 7.47-7.43 (m, 2H), 7.37 (d, | | | |
| | 1H), 3.87-3.75 (m, 1H), 3.72-3.64 (m, 1H), 3.52- | | | |
| | 3.34 (m, 1H), 3.26-3.11 (m, 2H), 2.99-2.86 (m, | | | |
| | 1H), 2.66-2.54 (m, 2H), 2.46 (s, 3H), 2.34-2.23 | | | |
| | (m, 2H) | | | |
| 249 | 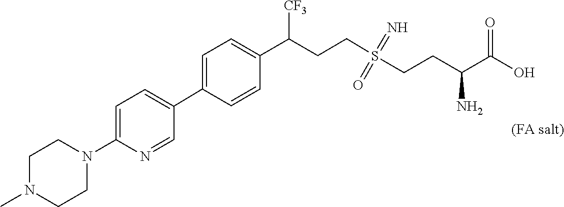 [SMILES: CN1CCN(c2ccc(-c3ccc(C(CCS(=N)(=O)CC[C@H](N)C(=O)O)C(F)(F)F)cc3)cn2)CC1] | 528.2 | 1.58 | C |
| | ¹H NMR (400 MHz, D₂O) δ 8.38 (d, 2H), 7.93 (br | | | |
| | d, 1H), 7.65 (d, 2H), 7.50 (br d, 2H), 6.98 (d, 1H), | | | |
| | 3.83-3.70 (m, 2H), 3.45-3.18 (m, 6H), 3.04-2.95 | | | |
| | (m, 1H), 2.90 (s, 3H), 2.63-2.39 (m, 2H), 2.30- | | | |
| | 2.20 (m, 2H) | | | |
| 250 | 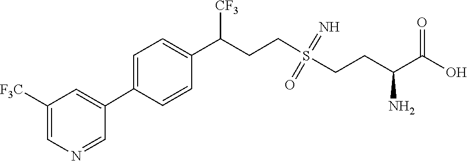 [SMILES: N=S(=O)(CCC(c1ccc(-c2cncc(C(F)(F)F)c2)cc1)C(F)(F)F)CC[C@H](N)C(=O)O] | 498.2 | 2.43 | B |
| | ¹H NMR (400 MHz, MeOD-d₄) δ 9.12 (s, 1H), 8.89 | | | |
| | (s, 1H), 8.40 (s, 1H), 7.83 (d, 2H), 7.60 (br d, 2H), | | | |
| | 3.90-3.78 (m, 1H), 3.72-3.62 (m, 1H), 3.36 (br s, | | | |
| | 1H), 3.27 (br s, 1H), 3.22-3.09 (m, 1H), 2.97- | | | |
| | 2.83 (m, 1H), 2.69-2.44 (m, 2H), 2.35-2.21 (m, | | | |
| | 2H) | | | |
| 251 | 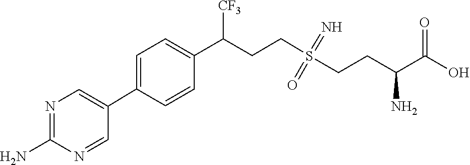 [SMILES: N=S(=O)(CCC(c1ccc(-c2cnc(N)nc2)cc1)C(F)(F)F)CC[C@H](N)C(=O)O] | 446.2 | 1.54 | C |
| | ¹H NMR (400 MHz, MeOD-d₄) δ 8.59 (s, 2H), 7.65 | | | |
| | (d, 2H), 7.51 (d, 2H), 3.86-3.62 (m, 2H), 3.47- | | | |
| | 3.35 (m, 1H), 3.31-3.22 (m, 1H), 3.22-3.09 (m, | | | |
| | 1H), 2.99-2.83 (m, 1H), 2.67-2.55 (m, 1H), 2.54- | | | |
| | 2.39 (m, 1H), 2.38-2.21 (m, 2H). | | | |
| 252 | 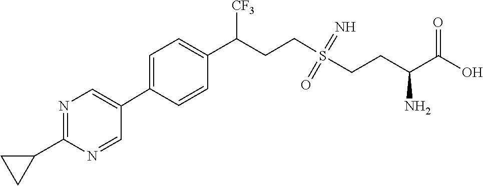 [SMILES: N=S(=O)(CCC(c1ccc(-c2cnc(C3CC3)nc2)cc1)C(F)(F)F)CC[C@H](N)C(=O)O] | 471.1 | 1.99 | C |
| | ¹H NMR (400 MHz, MeOD-d₄) δ 8.95-8.84 (m, | | | |
| | 2H), 7.82-7.70 (m, 2H), 7.61-7.50 (m, 2H), 3.88- | | | |
| | 3.73 (m, 1H), 3.72-3.61 (m, 1H), 3.43-3.34 (m, | | | |
| | 1H), 3.30-3.22 (m, 1H), 3.21-3.08 (m, 1H), 2.97- | | | |
| | 2.83 (m, 1H), 2.66-2.55 (m, 1H), 2.54-2.40 (m, | | | |
| | 1H), 2.36-2.17 (m, 3H), 1.22-1.08 (m, 4H) | | | |
| 253 | 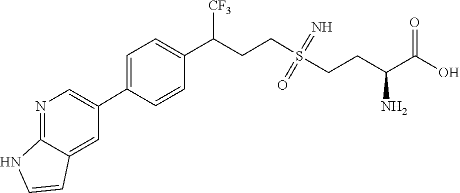 [SMILES: N=S(=O)(CCC(c1ccc(-c2cnc3[nH]ccc3c2)cc1)C(F)(F)F)CC[C@H](N)C(=O)O] | 469.2 | 2.22 | B |
| | ¹H NMR (400 MHz, MeOD-d₄) δ 8.46 (s, 1H), 8.25 | | | |
| | (s, 1H), 7.74 (d, 2H), 7.51 (d, 2H), 7.44 (d, 1H), | | | |
| | 6.57 (d, 1H), 3.86-3.62 (m, 2H), 3.34 (br s, 1H), | | | |
| | 3.28 (br s, 1H), 3.22-3.11 (m, 1H), 2.99-2.85 (m, | | | |
| | 1H), 2.67-2.42 (m, 2H), 2.34-2.21 (m, 2H) | | | |
| 254 | 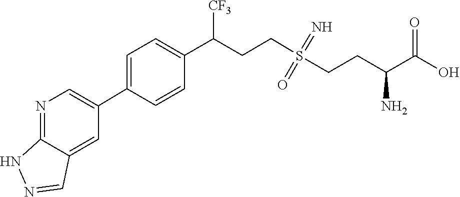 [SMILES: N=S(=O)(CCC(c1ccc(-c2cnc3[nH]ncc3c2)cc1)C(F)(F)F)CC[C@H](N)C(=O)O] | 470.2 | 1.96 | B |
| | ¹H NMR (400 MHz, MeOD-d₄) δ 8.84 (d, 1H), 8.51 | | | |
| | (d, 1H), 8.21 (s, 1H), 7.80 (d, 2H), 7.57 (d, 2H), | | | |
| | 3.82 (quin, 1H), 3.75-3.65 (m, 1H), 3.47-3.36 (m, | | | |
| | 1H), 3.31-3.25 (m, 1H), 3.24-3.12 (m, 1H), 3.02- | | | |
| | 2.86 (m, 1H), 2.69-2.45 (m, 2H), 2.39-2.22 (m, 2H) | | | |
| 255 | 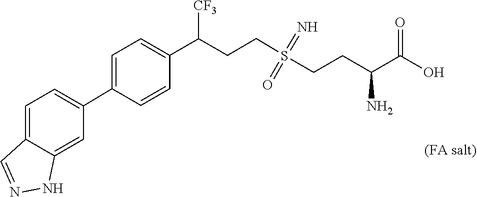 [SMILES: N=S(=O)(CCC(c1ccc(-c2ccc3cn[nH]c3c2)cc1)C(F)(F)F)CC[C@H](N)C(=O)O] | 469.1 | 2.02 | C |
| | ¹H NMR (400 MHz, MeOD-d4) δ 8.07 (d, 1H), | | | |
| | 7.85 (d, 1H), 7.78-7.74 (m, 3H), 7.53-7.44 (m, | | | |
| | 3H), 3.82-3.65 (m, 2H), 3.44-3.34 (m, 1H), 3.29- | | | |
| | 3.11 (m, 2H), 2.99-2.86 (m, 1H), 2.66-2.44 (m, | | | |
| | 2H), 2.35-2.24 (m, 2H). | | | |
| 256 | 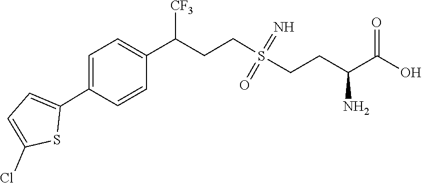 [SMILES: N=S(=O)(CCC(c1ccc(-c2ccc(Cl)s2)cc1)C(F)(F)F)CC[C@H](N)C(=O)O] | 469.0 | 2.29 | C |
| | ¹H NMR (400 MHz, MeOD-d₄) δ 7.63 (d, 2H), 7.43 | | | |
| | (d, 2H), 7.27 (d, 1H), 6.99 (d, 1H), 3.71-3.70 (m, | | | |
| | 1H), 3.71 (br s, 1H), 3.69-3.63 (m, 1H), 3.39- | | | |
| | 3.34 (m, 1H), 3.28-3.22 (m, 1H), 3.16-3.08 (m, | | | |
| | 1H), 2.96-2.77 (m, 1H), 2.67-2.66 (m, 1H), 2.64- | | | |
| | 2.52 (m, 1H), 2.50-2.37 (m, 1H), 2.32-2.20 (m, | | | |
| | 2H) | | | |
| 257 | 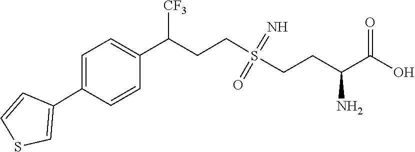 [SMILES: N=S(=O)(CCC(c1ccc(-c2ccsc2)cc1)C(F)(F)F)CC[C@H](N)C(=O)O] | 435.0 | 2.11 | C |
| | ¹H NMR (400 MHz, MeOD) δ 7.61 (d, 2H), 7.58- | | | |
| | 7.54 (m, 1H), 7.41-7.35 (m, 2H), 7.32 (d, 2H), | | | |
| | 3.69-3.50 (m, 2H), 3.32-3.23 (m, 1H), 3.18-2.97 | | | |
| | (m, 3H), 2.78 (br dd, 1H), 2.53-2.27 (m, 2H), 2.25- | | | |
| | 2.04 (m, 2H) | | | |
| 258 | 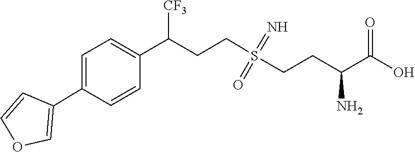 [SMILES: N=S(=O)(CCC(c1ccc(-c2ccoc2)cc1)C(F)(F)F)CC[C@H](N)C(=O)O] | 419.2 | 2.33 | B |
| | ¹H NMR (400 MHz, MeOD-d₄) δ 7.94 (s, 1H), 7.65- | | | |
| | 7.54 (m, 3H), 7.40 (d, 2H), 6.82 (s, 1H), 3.74- | | | |
| | 3.62 (m, 2H), 3.51-3.39 (m, 1H), 3.27 (br d, 1H), | | | |
| | 3.19-3.08 (m, 1H), 2.96-2.82 (m, 1H), 2.61-2.40 | | | |
| | (m, 2H), 2.28 (dt, 2H) | | | |
| 259 | 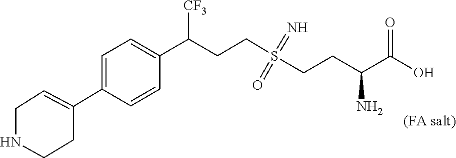 [SMILES: N=S(=O)(CCC(c1ccc(C2=CCNCC2)cc1)C(F)(F)F)CC[C@H](N)C(=O)O] | 434.1 | 1.48 | C |
| | ¹H NMR (400 MHz, D₂O) δ 8.41 (s, 1H), 7.60- | | | |
| | 7.53 (m, 2H), 7.47 (br d, 2H), 6.20 (br s, 1H), 3.91- | | | |
| | 3.82 (m, 3H), 3.79-3.71 (m, 1H), 3.50 (t, 2H), 3.39- | | | |
| | 3.20 (m, 3H), 3.03-2.94 (m, 1H), 2.81 (br s, 2H), | | | |
| | 2.62-2.54 (m, 1H), 2.49-2.40 (m, 1H), 2.27 (br | | | |
| | dd, 2H), 1.21 (s, 1H) | | | |
| 260 | 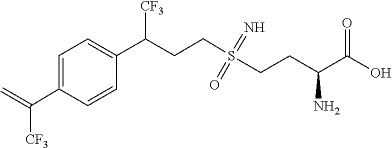 [SMILES: C=C(c1ccc(C(CCS(=N)(=O)CC[C@H](N)C(=O)O)C(F)(F)F)cc1)C(F)(F)F] | 447.1 | 2.07 | C |
| | ¹H NMR (400 MHz, MeOD-d₄) δ 7.62-7.53 (m, | | | |
| | 2H), 7.51-7.44 (m, 2H), 6.06 (d, 1H), 5.98 (s, 1H), | | | |
| | 3.87-3.74 (m, 1H), 3.73-3.63 (m, 1H), 3.51-3.35 | | | |
| | (m, 1H), 3.31-3.22 (m, 1H), 3.21-3.08 (m, 1H), | | | |
| | 2.98-2.84 (m, 1H), 2.67-2.55 (m, 1H), 2.53-2.40 | | | |
| | (m, 1H), 2.38-2.22 (m, 2H) | | | |
| 261 | 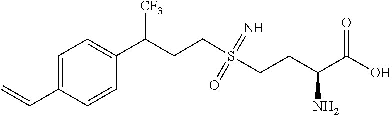 [SMILES: C=Cc1ccc(C(CCS(=N)(=O)CC[C@H](N)C(=O)O)C(F)(F)F)cc1] | 379.1 | 1.92 | C |
| | ¹H NMR (400 MHz, D₂O) δ 7.57 (d, 2H), 7.43 (d, | | | |
| | 2H), 6.81 (dd, 1H), 5.89 (d, , 1H), 5.35 (d, 1H), | | | |
| | 3.83 (q, 1H), 3.78-3.65 (m, 1H), 3.48-3.35 (m, | | | |
| | 1H), 3.34-3.21 (m, 2H), 3.08-2.94 (m, 1H), 2.63- | | | |
| | 2.51 (m, 1H), 2.49-2.36 (m, 1H), 2.34-2.20 (m, | | | |
| | 2H) | | | |
| 262 | 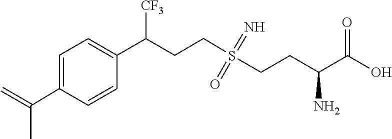 [SMILES: C=C(C)c1ccc(C(CCS(=N)(=O)CC[C@H](N)C(=O)O)C(F)(F)F)cc1] | 393.2 | 2.36 | B |
| | ¹H NMR (400 MHz, MeOD-d₄) δ 7.56 (br d, 2H), | | | |
| | 7.38 (br d, 2H), 5.43 (s, 1H), 5.14 (s, 1H), 3.77- | | | |
| | 3.60 (m, 2H), 3.31-3.07 (m, 3H), 2.97-2.76 (m, | | | |
| | 1H), 2.63-2.40 (m, 2H), 2.35-2.25 (m, 2H), 2.17 | | | |
| | (s, 3H) | | | |
| 263 | 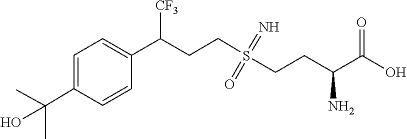 [SMILES: CC(C)(O)c1ccc(C(CCS(=N)(=O)CC[C@H](N)C(=O)O)C(F)(F)F)cc1] | 411.2 | 1.97 | B |
| | ¹H NMR (400 MHz, MeOD-d₄) δ 7.56 (d, 2H), 7.36 | | | |
| | (d, 2H), 3.76-3.56 (m, 2H), 3.32-3.05 (m, 3H), | | | |
| | 2.93-2.79 (m, 1H), 2.62-2.36 (m, 2H), 2.32-2.14 | | | |
| | (m, 2H), 1.55 (s, 6H) | | | |
| 264 | 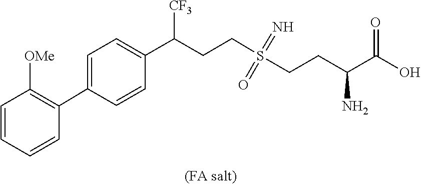 [SMILES: COc1ccccc1-c1ccc(C(CCS(=N)(=O)CC[C@H](N)C(=O)O)C(F)(F)F)cc1] | 459.2 | 2.10 | C |
| | ¹H NMR (400 MHz, DMSO-d₆) δ 7.55 (br d, 2H), | | | |
| | 7.43 (br d, 2H), 7.39-7.30 (m, 2H), 7.13 (d, 1H), | | | |
| | 7.04 (t, 1H), 4.13-3.87 (m, 2H), 3.79 (s, 3H), 3.25- | | | |
| | 3.20 (m, 1H), 3.15-2.94 (m, 2H), 2.75-2.63 (m, | | | |
| | 1H), 2.45-2.26 (m, 2H), 2.16-1.88 (m, 2H) | | | |
| 265 | 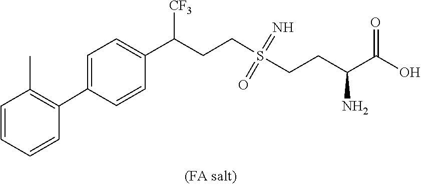 [SMILES: Cc1ccccc1-c1ccc(C(CCS(=N)(=O)CC[C@H](N)C(=O)O)C(F)(F)F)cc1] | 443.1 | 2.20 | C |
| | ¹H NMR (400 MHz, MeOD-d₄) δ = 7.50-7.42 (m, | | | |
| | 2H), 7.41-7.34 (m, 2H), 7.29-7.18 (m, 4H), 3.82- | | | |
| | 3.66 (m, 2H), 3.43-3.33 (m, 2H), 3.23-3.11 (m, | | | |
| | 1H), 3.02-2.85 (m, 1H), 2.63-2.44 (m, 2H), 2.35- | | | |
| | 2.28 (m, 2H), 2.26 (s, 3H) | | | |
| 266 | 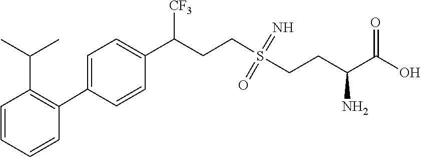 [SMILES: CC(C)c1ccccc1-c1ccc(C(CCS(=N)(=O)CC[C@H](N)C(=O)O)C(F)(F)F)cc1] | 471.1 | 2.34 | C |
| | ¹H NMR (400 MHz, MeOD-d₄) δ 7.48-7.39 (m, | | | |
| | 3H), 7.36-7.30 (m, 3H), 7.20 (dt, 1H), 7.15-7.09 | | | |
| | (m, 1H), 3.83-3.66 (m, 2H), 3.46-3.34 (m, 1H), | | | |
| | 3.29-3.09 (m, 2H), 3.08-2.89 (m, 2H), 2.67-2.45 | | | |
| | (m, 2H), 2.37-2.23 (m, 2H), 1.16 (dd, 6H) | | | |
| 267 | 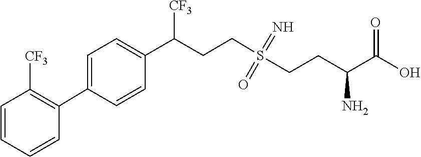 [SMILES: N=S(=O)(CCC(c1ccc(-c2ccccc2C(F)(F)F)cc1)C(F)(F)F)CC[C@H](N)C(=O)O] | 497.1 | 2.25 | C |
| | ¹H NMR (400 MHz, MeOD-d₄) δ 7.79 (d, 1H), 7.69- | | | |
| | 7.63 (m, 1H), 7.61-7.52 (m, 1H), 7.50-7.44 (m, | | | |
| | 2H), 7.42-7.35 (m, 3H), 3.87-3.75 (m, 1H), 3.73- | | | |
| | 3.63 (m, 1H), 3.44-3.34 (m, 2H), 3.23-3.09 (m, | | | |
| | 1H), 3.03-2.85 (m, 1H), 2.69-2.55 (m, 1H), 2.54- | | | |
| | 2.42 (m, 1H), 2.40-2.22 (m, 2H) | | | |
| 268 | 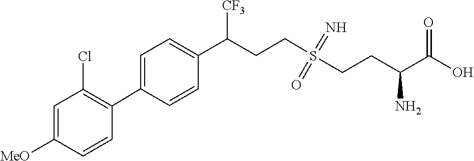 [SMILES: COc1ccc(-c2ccc(C(CCS(=N)(=O)CC[C@H](N)C(=O)O)C(F)(F)F)cc2)c(Cl)c1] | 493.1 | 2.25 | C |
| | ¹H NMR (400 MHz, MeOD-d₄) δ 7.52-7.44 (m, | | | |
| | 4H), 7.32 (dd, 1H), 7.09 (d, 1H), 6.98 (dd, 1H), | | | |
| | 3.86 (s, 3H), 3.83-3.74 (m, 1H), 3.73-3.65 (m, | | | |
| | 1H), 3.46-3.35 (m, 1H), 3.31-3.24 (m, 1H), 3.24- | | | |
| | 3.13 (m, 1H), 3.02-2.88 (m, 1H), 2.67-2.56 (m, | | | |
| | 1H), 2.56-2.42 (m, 1H), 2.38-2.22 (m, 2H). | | | |
| 269 | 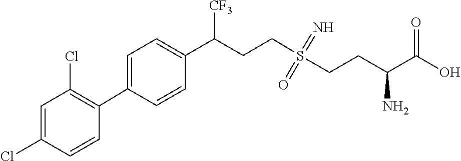 [SMILES: N=S(=O)(CCC(c1ccc(-c2ccc(Cl)cc2Cl)cc1)C(F)(F)F)CC[C@H](N)C(=O)O] | 497.1 | 2.30 | C |
| | ¹H NMR (400 MHz, MeOD-d₄) δ = 7.58 (d, 1H), | | | |
| | 7.50 (s, 4H), 7.44-7.35 (m, 2H), 3.82-3.65 (m, | | | |
| | 2H), 3.44-3.33 (m, 1H), 3.29-3.07 (m, 2H), 3.03- | | | |
| | 2.83 (m, 1H), 2.64-2.44 (m, 2H), 2.35-2.24 (m, | | | |
| | 2H). | | | |
| 270 | 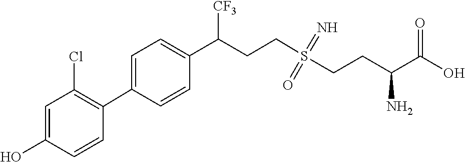 [SMILES: N=S(=O)(CCC(c1ccc(-c2ccc(O)cc2Cl)cc1)C(F)(F)F)CC[C@H](N)C(=O)O] | 479.1 | 2.03 | C |
| | ¹H NMR (400 MHz, MeOD-d₄) δ 7.55-7.35 (m, | | | |
| | 4H), 7.20 (d, 1H), 6.92 (d, 1H), 6.80 (dd, 1H), 3.86- | | | |
| | 3.57 (m, 2H), 3.38 (br d, 1H), 3.29-3.11 (m, 2H), | | | |
| | 3.05-2.81 (m, 1H), 2.66-2.41 (m, 2H), 2.39-2.15 | | | |
| | (m, 2H). | | | |
| 271 | 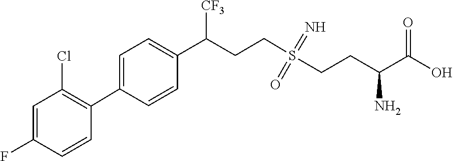 [SMILES: N=S(=O)(CCC(c1ccc(-c2ccc(F)cc2Cl)cc1)C(F)(F)F)CC[C@H](N)C(=O)O] | 481.1 | 2.61 | B |
| | ¹H NMR (400 MHz, MeOD-d₄) δ 7.48 (s, 4H), 7.42 | | | |
| | (ddd, 1H), 7.34 (dd, 1H), 7.17 (dt, 1H), 3.85-3.73 | | | |
| | (m, 1H), 3.72-3.63 (m, 1H), 3.34 (br d, 1H), 3.22 | | | |
| | (s, 1H), 3.20-3.11 (m, 1H), 3.00-2.88 (m, 1H), | | | |
| | 2.66-2.44 (m, 2H), 2.35-2.22 (m, 2H) | | | |
| 272 | 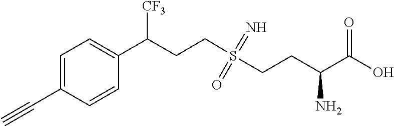 [SMILES: C#Cc1ccc(C(CCS(=N)(=O)CC[C@H](N)C(=O)O)C(F)(F)F)cc1] | 377.1 | 3.37 | E |
| | ¹H NMR (400 MHz, D₂O) δ 7.59 (d, 2H), 7.42 (d, | | | |
| | 2H), 3.87-3.69 (m, 2H), 3.53 (s, 1H), 3.45-3.19 | | | |
| | (m, 3H), 3.04-2.93 (m, 1H), 2.60-2.50 (m, 1H), | | | |
| | 2.46-2.36 (m, 1H), 2.26 (br dd, 2H) | | | |
| 273 | 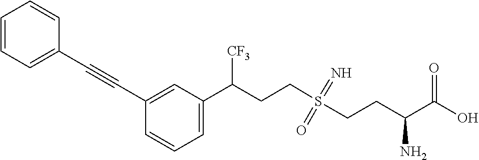 [SMILES: N=S(=O)(CCC(c1cccc(C#Cc2ccccc2)c1)C(F)(F)F)CC[C@H](N)C(=O)O] | 453.2 | 2.22 | C |
| | ¹H NMR (400 MHz, MeOD-d₄) δ 7.58-7.50 (m, | | | |
| | 4H), 7.48-7.36 (m, 5H), 3.83-3.60 (m, 2H), 3.45- | | | |
| | 3.33 (m, 1H), 3.30-3.21 (m, 1H), 3.20-3.09 (m, | | | |
| | 1H), 2.98-2.81 (m, 1H), 2.66-2.39 (m, 2H), 2.37- | | | |
| | 2.21 (m, 2H) | | | |
| 274 | 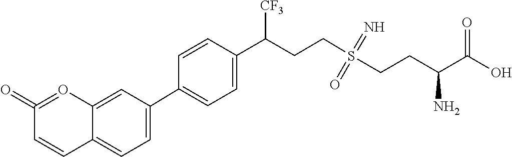 | 497.1 | 2.07 | C |
| | ¹H NMR (400 MHz, MeOD-d₄) δ 8.00 (d, 1H), 7.80 | | | |
| | (d, 2H), 7.75-7.71 (m, 1H), 7.69-7.64 (m, 2H), | | | |
| | 7.54 (d, 2H), 6.45 (d, 1H), 4.58 (s, 1H), 3.86-3.72 | | | |
| | (m, 1H), 3.71-3.59 (m, 1H), 3.35 (br d, 1H), 3.28- | | | |
| | 3.10 (m, 2H), 3.01-2.83 (m, 1H), 2.68-2.55 (m, | | | |
| | 1H), 2.54-2.41 (m, 1H), 2.37-2.19 (m, 2H) | | | |
| 275 | 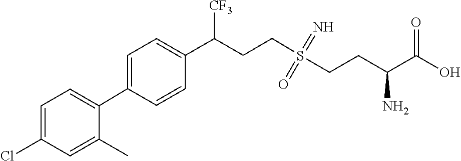 [SMILES: Cc1cc(Cl)ccc1-c1ccc(C(CCS(=N)(=O)CC[C@H](N)C(=O)O)C(F)(F)F)cc1] | 477.1 | 2.31 | C |
| | ¹H NMR (400 MHz, MeOD-d₄) δ 7.51-7.44 (m, | | | |
| | 2H), 7.38 (d, 2H), 7.31 (d, 1H), 7.28-7.22 (m, 1H), | | | |
| | 7.21-7.15 (m, 1H), 3.86-3.63 (m, 2H), 3.45-3.33 | | | |
| | (m, 1H), 3.30-3.09 (m, 2H), 3.01-2.85 (m, 1H), | | | |
| | 2.67-2.40 (m, 2H), 2.37-2.20 (m, 5H) | | | |
| 276 | 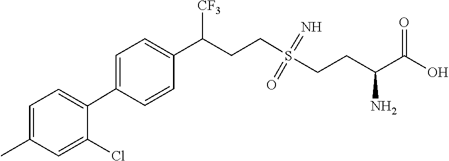 [SMILES: Cc1ccc(-c2ccc(C(CCS(=N)(=O)CC[C@H](N)C(=O)O)C(F)(F)F)cc2)c(Cl)c1] | 477.1 | 2.32 | C |
| | ¹H NMR (400 MHz, MeOD-d₄) δ 7.52-7.41 (m, | | | |
| | 4H), 7.33 (s, 1H), 7.29-7.23 (m, 1H), 7.22-7.17 | | | |
| | (m, 1H), 3.87-3.60 (m, 2H), 3.50-3.34 (m, 1H), | | | |
| | 3.29-3.10 (m, 2H), 2.03-2.84 (m, 1H), 2.67-2.43 | | | |
| | (m, 2H), 2.38 (s, 3H), 2.35-2.20 (m, 2H) | | | |
| 277 | 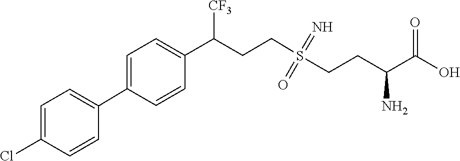 [SMILES: N=S(=O)(CCC(c1ccc(-c2ccc(Cl)cc2)cc1)C(F)(F)F)CC[C@H](N)C(=O)O] | 463.1 | 2.24 | C |
| | ¹H NMR (400 MHz, MeOD-d4) δ 7.77-7.58 (m, | | | |
| | 4H), 7.57-7.42 (m, 4H), 4.23 (tdd, 1H), 4.13-3.75 | | | |
| | (m, 4H), 3.64-3.37 (m, 1H), 2.80-2.36 (m, 4H). | | | |
| 278 | 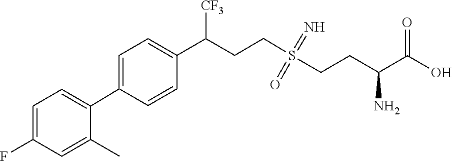 [SMILES: Cc1cc(F)ccc1-c1ccc(C(CCS(=N)(=O)CC[C@H](N)C(=O)O)C(F)(F)F)cc1] | 461.1 | 2.26 | C |
| | ¹H NMR (400 MHz, MeOD-d₄) δ 7.50-7.43 (m, | | | |
| | 2H), 7.36 (d, 2H), 7.20 (dd, 1H), 7.03 (dd, 1H), | | | |
| | 6.97 (dt, 1H), 3.83-3.64 (m, 2H), 3.45-3.34 (m, | | | |
| | 1H), 3.29-3.11 (m, 2H), 3.01-2.86 (m, 1H), 2.65- | | | |
| | 2.43 (m, 2H), 2.37-2.22 (m, 5H). | | | |
| 279 | 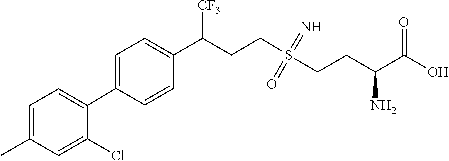 [SMILES: Cc1ccc(-c2ccc(C(CCS(=N)(=O)CC[C@H](N)C(=O)O)C(F)(F)F)cc2)c(Cl)c1] | 461.1 | 2.26 | C |
| | ¹H NMR (400 MHz, MeOD-d₄) δ 7.59 (d, 2H), 7.47 | | | |
| | (d, 2H), 7.38 (t, 1H), 7.13-6.98 (m, 2H), 3.83- | | | |
| | 3.63 (m, 2H), 3.43-3.33 (m, 1H), 3.29-3.09 (m, | | | |
| | 2H), 2.99-2.83 (m, 1H), 2.65-2.43 (m, 2H), 2.39 | | | |
| | (s, 3H), 2.35-2.23 (m, 2H) | | | |
| 280 | 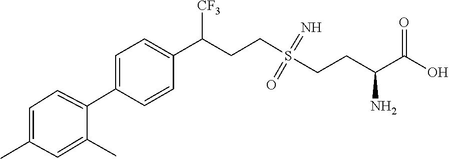 [SMILES: Cc1ccc(-c2ccc(C(CCS(=N)(=O)CC[C@H](N)C(=O)O)C(F)(F)F)cc2)c(C)c1] | 457.2 | 2.28 | C |
| | ¹H NMR (400 MHz, MeOD-d₄) δ 7.45-7.42 (m, | | | |
| | 2H), 7.38-7.34 (m, 2H), 7.10-7.03 (m, 3H), 3.82- | | | |
| | 3.73 (m, 1H), 3.71-3.65 (m, 1H), 3.41-3.36 (m, | | | |
| | 1H), 3.22-3.14 (m, 1H), 2.99-2.89 (m, 1H), 2.64- | | | |
| | 2.43 (m, 3H), 2.34 (s, 3H), 2.32-2.26 (m, 2H), | | | |
| | 2.23 (s, 3H) | | | |
| 281 | 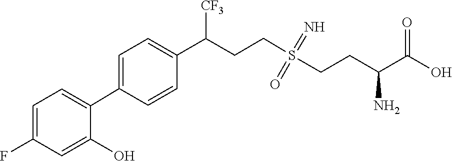 [SMILES: N=S(=O)(CCC(c1ccc(-c2ccc(F)cc2O)cc1)C(F)(F)F)CC[C@H](N)C(=O)O] | 463.1 | 2.09 | C |
| | ¹H NMR (400 MHz, DMSO-d₆) δ 7.57 (br d, 2H), | | | |
| | 7.40 (br d, 2H), 7.32 (dd, 1H), 6.78-6.63 (m, 2H), | | | |
| | 4.01-3.87 (m, 1H), 3.21 (br s, 2H), 3.15-2.94 (m, | | | |
| | 2H), 2.80-2.60 (m, 1H), 2.40-2.28 (m, 2H), 2.15- | | | |
| | 1.83 (m, 2H) | | | |
| 282 | 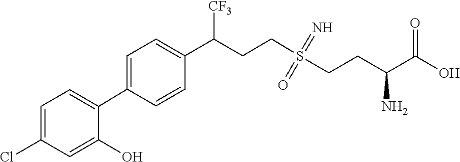 [SMILES: N=S(=O)(CCC(c1ccc(-c2ccc(Cl)cc2O)cc1)C(F)(F)F)CC[C@H](N)C(=O)O] | 479.1 | 2.18 | C |
| | ¹H NMR (400 MHz, MeOD-d₄) δ 7.61 (d, 2H), 7.42 | | | |
| | (d, 2H), 7.25 (d, 1H), 6.96-6.83 (m, 2H), 3.76- | | | |
| | 3.65 (m, 2H), 3.43-3.33 (m, 1H), 3.29-3.10 (m, | | | |
| | 2H), 3.03-2.84 (m, 1H), 2.63-2.42 (m, 2H), 2.34- | | | |
| | 2.23 (m, 2H) | | | |
| 283 | 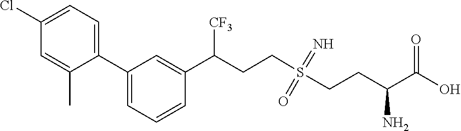 [SMILES: Cc1cc(Cl)ccc1-c1cccc(C(CCS(=N)(=O)CC[C@H](N)C(=O)O)C(F)(F)F)c1] | 477.1 | 2.72 | B |
| | ¹H NMR (400 MHz, MeOD-d₄) δ 7.54-7.47 (m, | | | |
| | 1H), 7.41 (br d, 1H), 7.37-7.29 (m, 3H), 7.27- | | | |
| | 7.22 (m, 1H), 7.22-7.16 (m, 1H), 3.85-3.73 (m, | | | |
| | 1H), 3.71-3.60 (m, 1H), 3.43-3.33 (m, 1H), 3.29- | | | |
| | 3.09 (m, 2H), 2.98-2.84 (m, 1H), 2.62-2.40 (m, | | | |
| | 2H), 2.36-2.25 (m, 2H), 2.24 (s, 3H). | | | |
| 284 | 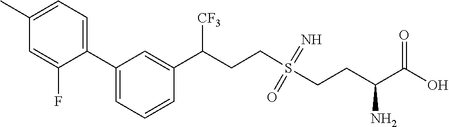 [SMILES: Cc1ccc(-c2cccc(C(CCS(=N)(=O)CC[C@H](N)C(=O)O)C(F)(F)F)c2)c(F)c1] | 461.1 | 2.54 | B |
| | ¹H NMR (400 MHz, MeOD-d₄) δ 7.57-7.47 (m, | | | |
| | 3H), 7.42-7.34 (m, 2H), 7.09 (d, 1H), 7.03 (d, 1H), | | | |
| | 3.82-3.72 (m, 1H), 3.69-3.62 (m, 1H), 3.41-3.32 | | | |
| | (m, 1H), 3.27-3.10 (m, 2H), 2.97-2.83 (m, 1H), | | | |
| | 2.64-2.44 (m, 2H), 2.39 (s, 3H), 2.32-2.20 (m, | | | |
| | 2H) | | | |
| 285 | 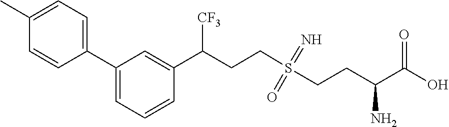 [SMILES: Cc1ccc(-c2cccc(C(CCS(=N)(=O)CC[C@H](N)C(=O)O)C(F)(F)F)c2)cc1] | 443.1 | 2.25 | C |
| | ¹H NMR (400 MHz, DMSO-d₆) δ = 7.69-7.61 (m, | | | |
| | 2H), 7.60-7.48 (m, 3H), 7.42-7.22 (m, 3H), 3.99 | | | |
| | (br s, 2H), 3.27-3.21 (m, 2H), 3.15-2.93 (m, 2H), | | | |
| | 2.71-2.59 (m, 1H), 2.45-2.38 (m, 1H), 2.35 (s, | | | |
| | 3H), 2.16-1.85 (m, 2H) | | | |
| 286 | 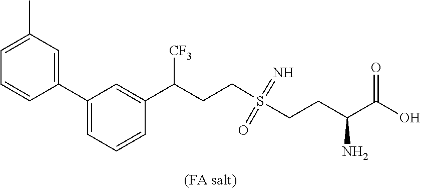 [SMILES: Cc1cccc(-c2cccc(C(CCS(=N)(=O)CC[C@H](N)C(=O)O)C(F)(F)F)c2)c1] | 443.1 | 2.23 | |
| | 1H NMR (400 MHz, MeOD-d₄) δ = 7.63 (br d, | | | |
| | 1H), 7.60 (br s, 1H), 7.50 (t, 1H), 7.44 (s, 1H), 7.41 | | | |
| | (br d, 1H), 7.38 (br d, 1H), 7.33 (t, 1H), 7.18 (br d, | | | |
| | 1H), 3.78 (dt, 1H), 3.69-3.62 (m, 1H), 3.27-3.14 | | | |
| | (m, 2H), 2.95-2.84 (m, 1H), 2.68-2.43 (m, 3H), | | | |
| | 2.41 (s, 3H), 2.32-2.24 (m, 2H). | | | |
| 287 | 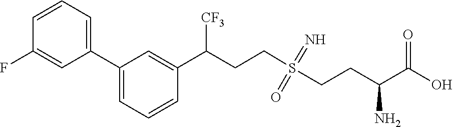 [SMILES: N=S(=O)(CCC(c1cccc(-c2cccc(F)c2)c1)C(F)(F)F)CC[C@H](N)C(=O)O] | 447.1 | 2.18 | C |
| | ¹H NMR (400 MHz, MeOD-d₄) δ 7.69-7.62 (m, | | | |
| | 2H), 7.56-7.36 (m, 5H), 7.14-7.07 (m, 1H), 3.88- | | | |
| | 3.73 (m, 1H), 3.71-3.62 (m, 1H), 3.44-3.32 (m, | | | |
| | 1H), 3.28-3.10 (m, 2H), 2.98-2.83 (m, 1H), 2.67- | | | |
| | 2.43 (m, 2H), 2.35-2.21 (m, 2H) | | | |
| 288 | 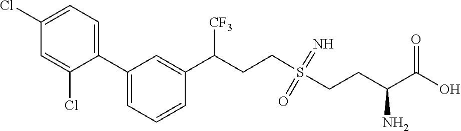 [SMILES: N=S(=O)(CCC(c1cccc(-c2ccc(Cl)cc2Cl)c1)C(F)(F)F)CC[C@H](N)C(=O)O] | 497.0 | 2.32 | C |
| | ¹H NMR (400 MHz, MeOD-d₄) δ 7.58 (d, 1H), 7.56- | | | |
| | 7.49 (m, 1H), 7.46 (s, 2H), 7.44 (s, 1H), 7.43- | | | |
| | 7.37 (m, 2H), 3.87-3.72 (m, 1H), 3.72-3.61 (m, | | | |
| | 1H), 3.46-3.34 (m, 1H), 3.28-3.09 (m, 2H), 3.00- | | | |
| | 2.84 (m, 1H), 2.66-2.39 (m, 2H), 2.37-2.20 (m, | | | |
| | 2H) | | | |
| 289 | 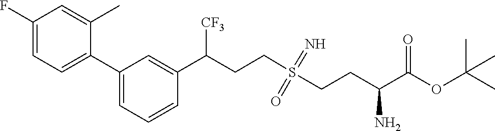 [SMILES: Cc1cc(F)ccc1-c1cccc(C(CCS(=N)(=O)CC[C@H](N)C(=O)OC(C)(C)C)C(F)(F)F)c1] | 461.1 | 2.24 | C |
| | ¹H NMR (400 MHz, MeOD-d₄) δ 7.52-7.47 (m, | | | |
| | 1H), 7.42-7.38 (m, 1H), 7.35-7.31 (m, 2H), 7.24- | | | |
| | 7.19 (m, 1H), 7.06-6.94 (m, 2H), 3.84-3.63 (m, | | | |
| | 2H), 3.43-3.33 (m, 1H), 3.29-3.12 (m, 2H), 2.99- | | | |
| | 2.85 (m, 1H), 2.64-2.40 (m, 2H), 2.35-2.26 (m, | | | |
| | 2H), 2.25 (s, 3H). | | | |
| 290 | 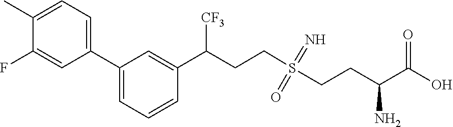 [SMILES: Cc1ccc(-c2cccc(C(CCS(=N)(=O)CC[C@H](N)C(=O)O)C(F)(F)F)c2)cc1F] | 461.1 | 2.28 | C |
| | ¹H NMR (400 MHz, MeOD-d₄) δ 7.69-7.59 (m, | | | |
| | 2H), 7.52 (t, 1H), 7.42 (br d, 1H), 7.39-7.29 (m, | | | |
| | 3H), 3.90-3.73 (m, 1H), 3.72-3.62 (m, 1H), 3.45- | | | |
| | 3.34 (m, 1H), 3.31-3.11 (m, 2H), 3.01-2.83 (m, | | | |
| | 1H), 2.69-2.42 (m, 2H), 2.40-2.20 (m, 5H). | | | |
| 291 | 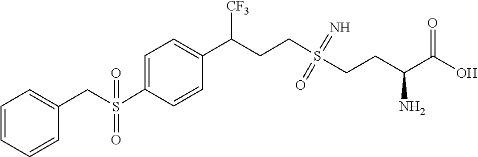 [SMILES: N=S(=O)(CCC(c1ccc(S(=O)(=O)Cc2ccccc2)cc1)C(F)(F)F)CC[C@H](N)C(=O)O] | 507.1 | 2.24 | B |
| | ¹H NMR (400 MHz, MeOD-d₄) δ 7.73 (d, 2H), 7.60 | | | |
| | (d, 2H), 7.37-7.23 (m, 3H), 7.17-7.09 (m, 2H), | | | |
| | 4.54 (s, 2H), 3.99-3.82 (m, 1H), 3.77-3.60 (m, | | | |
| | 1H), 3.44-3.34 (m, 1H), 3.30-3.22 (m, 1H), 3.19- | | | |
| | 3.04 (m, 1H), 2.90-2.74 (m, 1H), 2.70-2.56 (m, | | | |
| | 1H), 2.54-2.39 (m, 1H), 2.37-2.21 (m, 2H) | | | |

### Exemplary Embodiment 1aa18

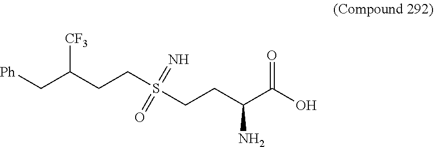 [SMILES: N=S(=O)(CCC(Cc1ccccc1)C(F)(F)F)CC[C@H](N)C(=O)O]

### (2S)-2-amino-4-(3-benzyl-4,4,4-trifluorobutylsulfonimnidoyl)butanoic acid

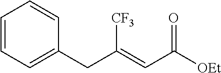 [SMILES: CCOC(=O)/C=C(/Cc1ccccc1)C(F)(F)F]

To a solution of ethyl 2-(diethoxyphosphoryl)acetate (893 ing, 3.99 iniol, 790 μL, 1.50 eq) in THE (6.00 mnL) at 0° C. was added NaH (127 ing, 3.19 iniol, 60.0 wt. %, 1.20 eq) and the mixture was stirred at 0-15° C. for 0.5 h. Then 1,1,1-trifluoro-3-phenylpropan-2-one (500 mg, 2.66 mmol, 409 μL, 1.00 eq) was added and the mixture was stirred at 15° C. for 2.5 h. Water (15 mL) was then added at 0° C. and the mixture was extracted with EtOAc (2×15 mL). The combined organic layers were dried over Na₂SO₄, filtered, and concentrated under reduced pressure. The residue was purified by column chromatography (SiO₂, petroleum ether/EtOAc=1:0 to 5:1) to give (0.4 g, 1.39 mmol, 52.41% yield) as a colorless oil. ¹H NMR (400 MHz, CDCl₃-d) δ 7.44-7.31 (m, 4H), 7.30 (d, 4H), 7.26-7.17 (m, 12H), 6.50 (s, 3H), 5.77 (s, 1H), 4.29-4.17 (m, 8H), 4.11 (s, 6H), 3.59 (s, 2H), 1.33-1.28 (m, 12H).

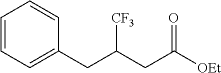 [SMILES: CCOC(=O)CC(Cc1ccccc1)C(F)(F)F]

To a solution of ethyl 3-benzyl-4,4,4-trifluorobut-2-enoate (400 mg, 1.55 mmol, 1.00 eq) in EtOH (5 mL) was added Pd/C (40.0 mg, 10% purity). The mixture was stirred at 20° C. for 2 h under H2 (15 psi). The reaction mixture was filtered and concentrated under reduced pressure. The residue was purified by column chromatography (SiO₂, petroleum ether/EtOAc=1:0 to 1:1) to obtain ethyl 3-benzyl-4,4,4-trifluorobutanoate (380 mg, 1.31 mmol, 84.8% yield) as a colorless oil. ¹H NMR (400 MHz, CDCl₃-d) δ 7.36-7.29 (m, 2H), 7.26-7.20 (m, 3H), 4.02 (q, 2H), 3.16-3.04 (m, 2H), 2.71-2.51 (m, 2H), 2.37 (dd, 1H), 1.19 (t, 3H).

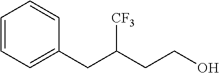 [SMILES: OCCC(Cc1ccccc1)C(F)(F)F]

To a solution of ethyl 3-benzyl-4,4,4-trifluorobutanoate (380 mg, 1.46 mmol, 1.00 eq) in THE (5.00 mL) was added LiAlH₄ (66.5 mg, 1.75 mmol, 1.20 eq) at 0° C. The mixture was stirred at 20° C. for 1 h before quenching with water (0.066 mL), 15% NaOH (0.066 mL), and additional water (0.19 mL). The mixture was diluted with EtOAc (10 mL), filtered, and concentrated under reduced pressure. The residue was purified by column chromatography (SiO₂, petroleum ether/EtOAc=1:0 to 5:1) to give 3-benzyl-4,4,4-trifluorobutan-1-ol (300 mg, 1.17 mmol, 80.0% yield) as colorless oil. ¹H NMR (400 MHz, CDCl₃-d) δ 7.37-7.29 (m, 2H), 7.27-7.19 (m, 3H), 3.60 (t, 2H), 3.15-3.06 (m, 1H), 2.68-2.58 (m, 2H), 1.96-1.80 (m, 1H), 1.76-1.62 (m, 1H).

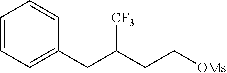 [SMILES: CS(=O)(=O)OCCC(Cc1ccccc1)C(F)(F)F]

To a solution of 3-benzyl-4,4,4-trifluorobutan-1-ol (300 mg, 1.37 mmol, 1.00 eq) in DCM (3.00 mL) at 0° C. was added TEA (287 μL, 2.06 mmol 1.50 eq) and methylsulfonyl methanesulfonate (287 mg, 1.65 mmol, 1.20 eq) under N₂. The mixture was warmed to 20° C. and stirred for 2 h. The reaction mixture was quenched by addition water (8 mL) at 0° C. and the mixture was extracted with DCM (15 mL×2). The combined organic layers were dried over Na₂SO₄, filtered, and concentrated under reduced pressure. The residue was purified by column chromatography (SiO₂, petroleum ether/EtOAc=1:0 to 5:1) to give 3-benzyl-4,4,4-trifluorobutyl methanesulfonate (330 mg, 1.00 mmol, 72.9% yield) as colorless oil. ¹H NMR (400 MHz, CDCl₃-d) δ 7.41-7.28 (m, 3H), 7.21 (d, 2H), 4.14 (t, 2H), 3.28-3.05 (m, 1H), 2.86 (s, 3H), 2.65-2.55 (m, 2H), 2.10-1.99 (m, 1H), 1.95-1.85 (m, 1H).

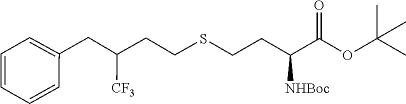 [SMILES: CC(C)(C)OC(=O)N[C@@H](CCSCCC(Cc1ccccc1)C(F)(F)F)C(=O)OC(C)(C)C]

To a solution of 3-benzyl-4,4,4-trifluorobutyl methanesulfonate (324 mg, 1.09 mmol, 1.10 eq) in DMF (3.00 mL) was added tert-butyl (tert-butoxycarbonyl)-L-homocysteinate (290 mg, 995 μmol, 1.00 eq), K₂CO₃ (412 mg, 2.99 mmol, 3.00 eq) and KI (330 mg, 1.99 mmol, 2.00 eq) and the mixture was stirred at 40° C. for 16 h. The reaction mixture was quenched with water (8 mL) at 0° C. and the mixture was extracted with EtOAc (15 mL×2). The combined organic layers were dried over Na₂SO₄, filtered, and concentrated under reduced pressure to give a residue. The residue was purified by column chromatography (SiO₂, petroleum ether/EtOAc=1:0 to 5:1) to give (2S)-tert-butyl 4-((3-benzyl-4,4,4-trifluorobutyl)thio)-2-((tert-butoxycarbonyl)amino)butanoate (600 mg, 915 μmol, 91.9% yield) as colorless oil. ¹H NMR (400 MHz, CDCl₃-d) δ 7.38-7.29 (m, 2H), 7.28 (br s, 1H), 7.21 (d, 2H), 5.00 (br d, 1H), 4.31-4.10 (m, 1H), 3.09 (br d, 1H), 2.71-2.39 (m, 4H), 2.37-2.27 (m, 2H), 2.04-1.81 (m, 2H), 1.80-1.64 (m, 2H), 1.50-1.42 (m, 18H).

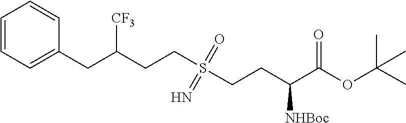 [SMILES: CC(C)(C)OC(=O)N[C@@H](CCS(=N)(=O)CCC(Cc1ccccc1)C(F)(F)F)C(=O)OC(C)(C)C]

To a solution of (2S)-tert-butyl 4-((3-benzyl-4,4,4-trifluorobutyl)thio)-2-((tert-butoxycarbonyl)amino)butanoate (600 mg, 1.22 mmol, 1.00 eq) in i-PrOH (6.00 mL) was added PhI(OAc)₂ (1.57 g, 4.88 mmol, 4.00 eq) and ammonium carbamate (762 mg, 9.76 mmol, 8.00 eq). The mixture was stirred at 15° C. for 16 h before concentrating under reduced pressure. The reaction mixture was quenched by addition of water (8 mL) at 0° C., and the mixture was extracted with DCM (15 mL×2). The combined organic layers were dried over Na₂SO₄, filtered, and concentrated under reduced pressure. The residue was purified by column chromatography (SiO₂, petroleum ether/EtOAc=1:0 to 3:1) to give (2S)-tert-butyl 4-(3-benzyl-4,4,4-trifluorobutylsulfonimidoyl)-2-((tert-butoxycarbonyl)amino)butanoate (400 mg, 688 μmol, 56.4% yield) as a white solid. ¹H NMR (400 MHz, CDCl₃-d) δ 7.38-7.28 (m, 3H), 7.22 (br d, 2H), 5.25-5.07 (m, 1H), 4.31-4.18 (m, 1H), 3.21-3.11 (m, 1H), 3.11-2.83 (m, 4H), 2.75-2.56 (m, 2H), 2.38-2.23 (m, 1H), 2.12-2.08 (m, 1H), 2.04-1.95 (m, 2H), 1.48 (s, 9H), 1.46 (s, 9H).

A solution of (2S)-tert-butyl 4-(3-benzyl-4,4,4-trifluorobutylsulfonimidoyl)-2-((tert-butoxycarbonyl)amino)butanoate (100 mg, 191 μmol, 1.00 eq) in HCl/dioxane (4.00 M, 3.00 mL, 62.7 eq) was stirred at 15° C. for 12 h. The reaction mixture was concentrated under reduced pressure and the residue was purified by prep-HPLC (column: C18-1 (150×30 mm, Sum); mobile phase: [water (FA)-MeCN]; gradient: 15-45% B, 10 min) to give (2S)-2-amino-4-(3-benzyl-4,4,4-trifluorobutylsulfonimidoyl)butanoic acid (25.0 mg, 60.6 μmol, 31.69% yield, FA). LCMS: Rt=1.962 min, (ES⁺) m/z (M+H)⁺=367.1, HPLC Conditions: C. ¹H NMR (400 MHz, D₂O) δ 7.38 (br d, 2H), 7.35-7.28 (m, 3H), 3.78 (br d, 1H), 3.36-3.03 (m, 5H), 2.89-2.77 (m, 1H), 2.75-2.63 (m, 1H), 2.27-2.15 (m, 2H), 1.88 (d, 2H).

### Exemplary Embodiment 1aa18.2

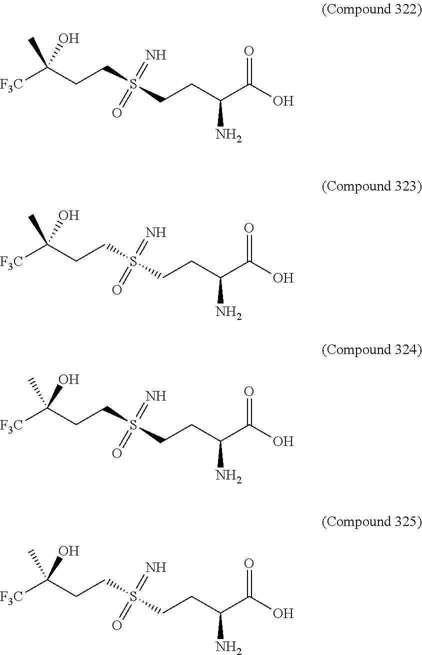 [SMILES: C[C@@](O)(CC[S@](=N)(=O)CC[C@H](N)C(=O)O)C(F)(F)F ; C[C@@](O)(CC[S@@](=N)(=O)CC[C@H](N)C(=O)O)C(F)(F)F ; C[C@](O)(CC[S@](=N)(=O)CC[C@H](N)C(=O)O)C(F)(F)F ; C[C@](O)(CC[S@@](=N)(=O)CC[C@H](N)C(=O)O)C(F)(F)F]

### Compound 322 (Isomer 1), Compound 323 (Isomer 2), Compound 324 (Isomer 3), & Compound 325 (Isomer 4)

### (S)-2-amino-4-((R,3R)-4,4,4-trifluoro-3-hydroxy-3-methylbutylsulfonimidoyl)butanoic acid (Isomer 1), (S)-2-amino-4-((S,3R)-4,4,4-trifluoro-3-hydroxy-3-methylbutylsulfonimidoyl)butanoic acid (Isomer 2), (S)-2-amino-4-((R,3S)-4,4,4-trifluoro-3-hydroxy-3-methylbutylsulfonimidoyl)butanoic acid (Isomer 3), & (S)-2-amino-4-((S,3S)-4,4,4-trifluoro-3-hydroxy-3-methylbutylsulfonimidoyl)butanoic acid (Isomer 4)

### Synthesis Stage 1

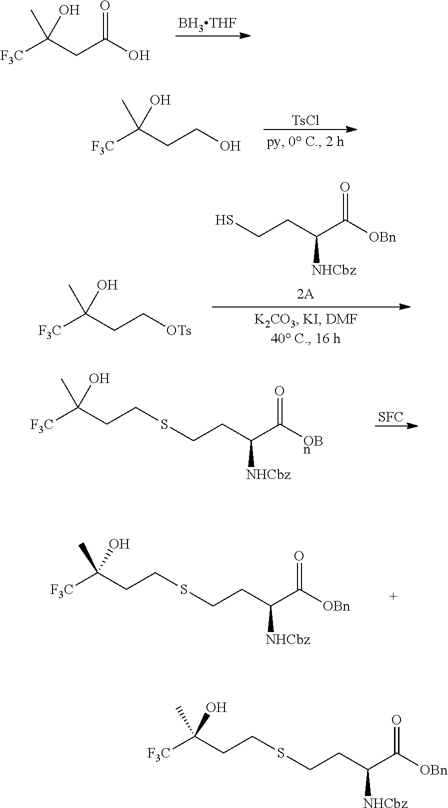

### Synthesis Stage 2

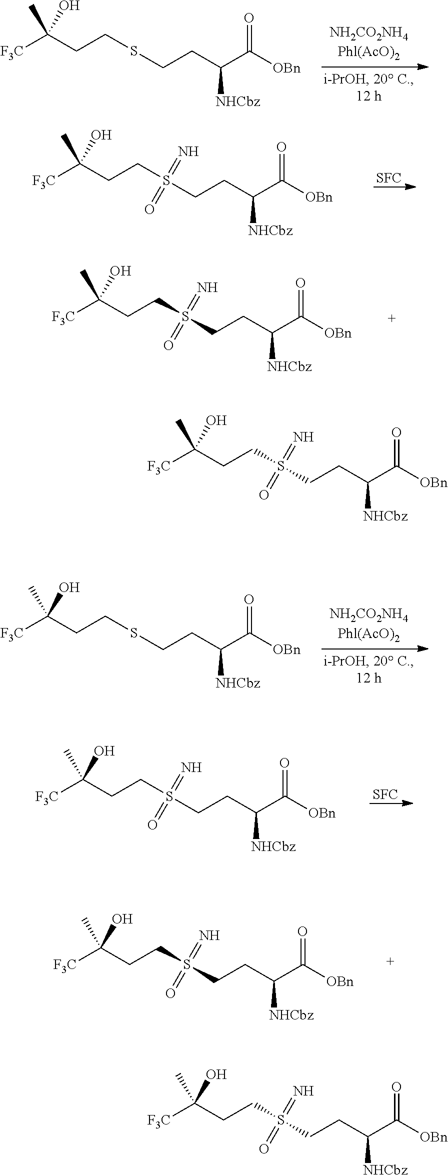

### Synthesis Stage 3

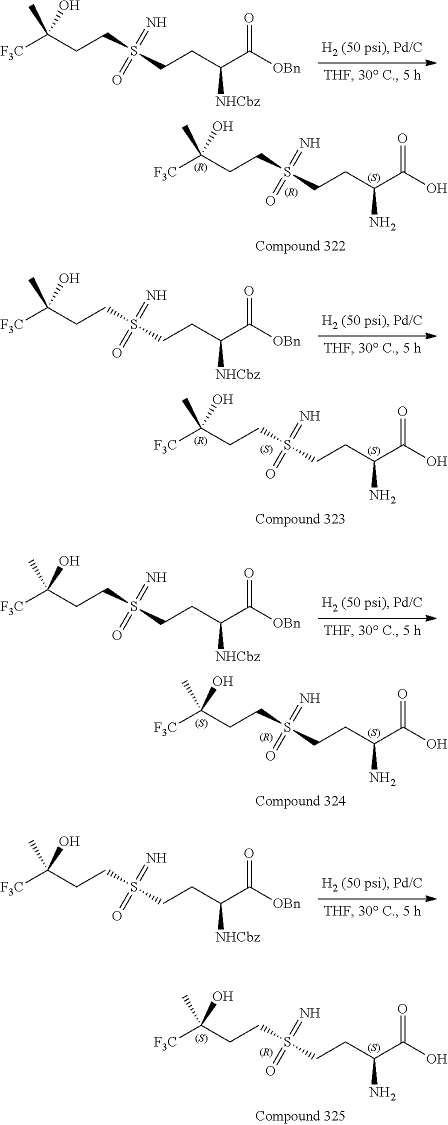

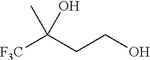

To a solution of 4,4,4-trifluoro-3-hydroxy-3-methylbutanoic acid (5.00 g, 29.5 mmol, 1 eq) in THF (80 mL) was added BH₃·THF (1 M, 43.5 mL, 1.5 eq) at 0° C. for 30 min. The mixture was stirred at 25° C. for 16 h under Ar. The mixture was poured into water (50 mL) at 0° C. and extracted with EtOAc (5 mL×3). The combined organic layers were dried over Na₂SO₄, filtered, and concentrated under reduced pressure. The residue was purified by column chromatography (SiO₂, petroleum ether/EtOAc=83:17) to give 4,4,4-trifluoro-3-methylbutane-1,3-diol (2.50 g, 15.8 mmol, 54.4% yield) as a yellow oil. 1H NMR (400 MHz, CDCl₃-d) δ 4.11-3.94 (m, 2H), 2.10 (ddd, 1H), 1.93-1.80 (m, 1H), 1.43 (s, 3H).

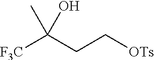 [SMILES: Cc1ccc(S(=O)(=O)OCCC(C)(O)C(F)(F)F)cc1]

To a solution of 4,4,4-trifluoro-3-methylbutane-1,3-diol (2.50 g, 15.8 mmol, 1 eq) in pyridine (20 mL) was added TsCl (4.52 g, 23.7 mmol, 1.5 eq) at 0° C. The mixture was stirred at 0° C. for 2 h. The mixture was diluted with EtOAc (20 mL) and washed with HCl (2M, 10 mL×2). The organic layer was washed with brine, dried over Na₂SO₄, filtered, and concentrated under reduced pressure. The residue was purified by column chromatography (SiO₂, petroleum ether/EtOAc=88:12) to give 4,4,4-trifluoro-3-hydroxy-3-methylbutyl 4-methylbenzenesulfonate (2.70 g, 8.65 mmol, 54.8% yield) as a colorless oil. ¹H NMR (400 MHz, CDCl₃-d) δ 7.81 (d, 2H), 7.37 (d, 2H), 4.36-4.19 (m, 2H), 2.47 (s, 3H), 2.19-2.11 (m, 1H), 2.07-1.97 (m, 1H), 1.39 (s, 3H).

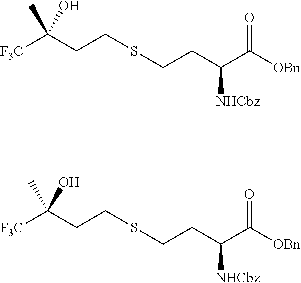 [SMILES: C[C@@](O)(CCSCC[C@H](NC(=O)OCc1ccccc1)C(=O)OCc1ccccc1)C(F)(F)F ; C[C@](O)(CCSCC[C@H](NC(=O)OCc1ccccc1)C(=O)OCc1ccccc1)C(F)(F)F]

To a solution of 4,4,4-trifluoro-3-hydroxy-3-methylbutyl 4-methylbenzenesulfonate (2.00 g, 5.56 mmol, 1 eq) and benzyl ((benzyloxy)carbonyl)-L-homocysteinate (2.09 g, 6.68 mmol, 1.2 eq) in DMF (20 mL) was added K₂CO₃ (2.31 g, 16.6 mmol, 3 eq) and KI (1.85 g, 11.3 mmol, 2 eq). The mixture was stirred at 25° C. for 16 h. The mixture was poured into water (50 mL) and extracted with EtOAc (25 mL×3). The combined organic layers were dried over Na₂SO₄, filtered, and concentrated under reduced pressure. The residue was purified by column chromatography (SiO₂, petroleum ether/EtOAc=90:10) to give benzyl N-((benzyloxy)carbonyl)-S-(4,4,4-trifluoro-3-hydroxy-3-methylbutyl)-L-homocysteinate, which was then separated by SFC (column: Daicel CHIRALPAK IF (250×30 mm, 10 μm); mobile phase: [water (0.1% NH₃)-MeOH]; gradient: 31% B, 6 min). The separated compounds are listed in order of elution. Benzyl N-((benzyloxy)carbonyl)-S—((R)-4,4,4-trifluoro-3-hydroxy-3-methylbutyl)-L-homocysteinate (1.00 g, 2.00 mmol, 35.98% yield) was obtained as a colorless oil. ¹H NMR (400 MHz, CDCl₃-d) δ 7.36 (br s, 10H), 5.43 (br d, 1H), 5.27-5.15 (m, 2H), 5.12 (s, 2H), 4.56 (br d, 1H), 4.11-3.96 (m, 1H), 2.73-2.47 (m, 5H), 2.24-2.09 (m, 1H), 2.05-1.92 (m, 2H), 1.90-1.77 (m, 1H), 1.22 (d, 4H). Benzyl N-((benzyloxy)carbonyl)-S—((S)-4,4,4-trifluoro-3-hydroxy-3-methylbutyl)-L-homocysteinate (1.00 g, 2.00 mmol, 35.98% yield) was obtained as a colorless oil. ¹H NMR (400 MHz, CDCl₃-d) δ 7.36 (br s, 10H), 5.44 (br d, 1H), 5.27-5.17 (m, 2H), 5.12 (s, 2H), 4.57 (br d, 1H), 2.74-2.43 (m, 5H), 2.25-2.08 (m, 1H), 2.07-1.92 (m, 2H), 1.91-1.76 (m, 1H), 1.34 (s, 3H).

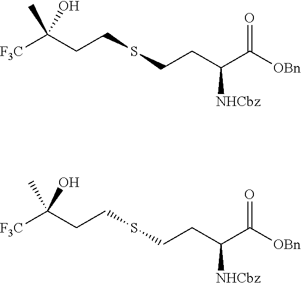

To a solution of benzyl N-((benzyloxy)carbonyl)-S—((R)-4,4,4-trifluoro-3-hydroxy-3-methylbutyl)-L-homocysteinate (1.00 g, 2.00 mmol, 1 eq) in i-PrOH (10 mL) was added PhI(OAc)₂ (2.58 g, 8.01 mmol, 4 eq) and ammonium carbamate (1.25 g, 16.1 mmol, 8 eq). The mixture was stirred at 25° C. for 16 h. The reaction mixture was concentrated under reduced pressure. The residue was diluted with water (20 mL) and extracted with EtOAc (15 mL×3). The combined organic layers were dried over Na₂SO₄, filtered, and concentrated under reduced pressure. The residue was purified by prep-TLC (SiO₂, petroleum ether/EtOAc=1:2, R_{f}=0.43) to give benzyl (2S)-2-(((benzyloxy)carbonyl)amino)-4-((3R)-4,4,4-trifluoro-3-hydroxy-3-methylbutylsulfonimidoyl)butanoate, which was then separated by SFC (column: Daicel CHIRALPAK IG (250×30 mm, 10 μm); mobile phase: [Hexane-IPA]; gradient: 40% B, 10 min). The separated compounds are listed in order of elution. Benzyl (S)-2-(((benzyloxy)carbonyl)amino)-4-((R,3R)-4,4,4-trifluoro-3-hydroxy-3-methylbutylsulfonimidoyl)butanoate (220 mg, 414.6 μmol, 20.7% yield) was obtained as a yellow oil. ¹H NMR (400 MHz, CDCl₃-d) δ 7.43-7.30 (m, 10H), 5.69 (br d, 1H), 5.30-5.03 (m, 4H), 4.54 (br d, 1H), 3.29-2.92 (m, 4H), 2.53-2.33 (m, 2H), 2.27-2.13 (m, 1H), 2.10-1.98 (m, 1H), 1.42-1.24 (m, 3H). Benzyl (S)-2-(((benzyloxy)carbonyl)amino)-4-((S,3R)-4,4,4-trifluoro-3-hydroxy-3-methylbutylsulfonimidoyl)butanoate (190 mg, 358.11 μmol, 17.89% yield) was obtained as a yellow oil. ¹H NMR (400 MHz, CDCl₃-d) δ 7.45-7.30 (m, 10H), 5.65 (br d, 1H), 5.29-5.04 (m, 4H), 4.53 (br d, 1H), 3.33-3.20 (m, 1H), 3.12 (br dd, 2H), 3.05-2.94 (m, 1H), 2.52-2.32 (m, 1H), 2.30-2.11 (m, 3H), 1.38 (s, 3H).

To a solution of benzyl (S)-2-(((benzyloxy)carbonyl)amino)-4-((R,3R)-4,4,4-trifluoro-3-hydroxy-3-methylbutylsulfonimidoyl)butanoate (180 mg, 339 μmol, 1 eq) in THF (6 mL) was added HCl (1 M, 679 μL, 2 eq) and Pd/C (800 mg, 10% purity). The mixture was stirred at 30° C. for 3 h under H2 (50 psi). The mixture was filtered, and the filtrate was concentrated under reduced pressure. The residue was purified by prep-HPLC (column: C18-1 (150×30 mm, 5 μm); mobile phase: [water (FA)-MeCN]; gradient: 1-10% B, 20 min) to give (S)-2-amino-4-((R,3R)-4,4,4-trifluoro-3-hydroxy-3-methylbutylsulfonimidoyl)butanoic acid (Isomer 1, 31.7 mg, 90.1 μmol, 26.5% yield, FA) as white solid. LCMS: Rt=0.689 min., (ES⁺) m/z (M+H)⁺=307.0, HPLC Conditions: A. ¹H NMR (400 MHz, D₂O) δ 3.86 (t, 1H), 3.52-3.31 (m, 4H), 2.41-2.31 (m, 2H), 2.27-2.09 (m, 2H), 1.38 (s, 3H).

To a solution of benzyl (S)-2-(((benzyloxy)carbonyl)amino)-4-((S,3R)-4,4,4-trifluoro-3-hydroxy-3-methylbutylsulfonimidoyl)butanoate (190 mg, 358 μmol, 1 eq) in THF (6 mL) was added HCl (1 M, 716 μL, 2 eq) and Pd/C (800 mg, 10% purity). The mixture was stirred at 30° C. for 3 h under H₂ (50 psi). The mixture was filtered and concentrated under reduced pressure. The residue was purified by prep-HPLC (column: C18-1 (150×30 mm, 5 μm); mobile phase: [water (FA)-MeCN]; gradient: 1-10% B, 10 min) to give (S)-2-amino-4-((S,3R)-4,4,4-trifluoro-3-hydroxy-3-methylbutylsulfonimidoyl)butanoic acid (Isomer 2, 23.8 mg, 77.8 μmol, 21.7% yield, 100% purity) as white solid. LCMS: Rt=0.640 min., (ES⁺) m/z (M+H)⁺=307.1, HPLC Conditions: A. ¹H NMR (400 MHz, D₂O) δ 3.86 (t, 1H), 3.52-3.31 (m, 4H), 2.41-2.31 (m, 2H), 2.27-2.09 (m, 2H), 1.38 (s, 3H).

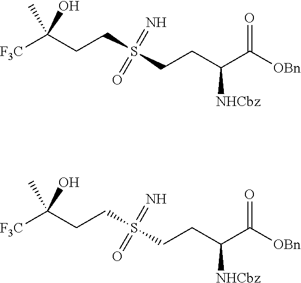 [SMILES: C[C@](O)(CC[S@](=N)(=O)CC[C@H](NC(=O)OCc1ccccc1)C(=O)OCc1ccccc1)C(F)(F)F ; C[C@](O)(CC[S@@](=N)(=O)CC[C@H](NC(=O)OCc1ccccc1)C(=O)OCc1ccccc1)C(F)(F)F]

To a solution of benzyl N-((benzyloxy)carbonyl)-S—((S)-4,4,4-trifluoro-3-hydroxy-3-methylbutyl)-L-homocysteinate (1.00 g, 2.00 mmol, 1 eq) in i-PrOH (10 mL) was added PhI(OAc)₂ (2.58 g, 8.01 mmol, 4 eq) and ammonium carbamate (1.25 g, 16.1 mmol, 8 eq). The mixture was stirred at 25° C. for 16 h. The reaction mixture was concentrated under reduced pressure and poured into water (20 mL) and extracted with EtOAc (15 mL×3). The combined organic layers were dried over Na₂SO₄, filtered, and concentrated under reduced pressure. The residue was purified by prep-TLC (SiO₂, petroleum ether/EtOAc=1:2, R_{f}=0.43) to give benzyl (2S)-2-(((benzyloxy)carbonyl)amino)-4-((3S)-4,4,4-trifluoro-3-hydroxy-3-methylbutylsulfonimidoyl)butanoate, which was then separated by SFC (column: ChiralPak IH, (250×30 mm, 10 μm); mobile phase: [Heptane-EtOH]; gradient: 20% B, 10 min). The separated compounds are listed in order of elution. Benzyl (S)-2-(((benzyloxy)carbonyl)amino)-4-((R,3S)-4,4,4-trifluoro-3-hydroxy-3-methylbutylsulfonimidoyl)butanoate (260 mg, 490.5 μmol, 24.8% yield) was obtained as a yellow oil. ¹H NMR (400 MHz, CDCl₃-d) δ 7.43-7.30 (m, 10H), 5.61 (br d, 1H), 5.28-5.03 (m, 4H), 4.54 (br d, 1H), 3.29 (br dd, 1H), 3.23-3.08 (m, 2H), 3.06-2.94 (m, 1H), 2.51-2.36 (m, 1H), 2.32-2.11 (m, 3H), 1.38 (s, 3H). Benzyl (S)-2-(((benzyloxy)carbonyl)amino)-4-((S,3S)-4,4,4-trifluoro-3-hydroxy-3-methylbutylsulfonimidoyl)butanoate (360 mg, 678.5 μmol, 33.9% yield) was obtained as a yellow oil. ¹H NMR (400 MHz, CDCl₃-d) δ 7.43-7.30 (m, 10H), 5.68 (br d, 1H), 5.27-5.04 (m, 4H), 4.53 (br d, 1H), 3.31-2.95 (m, 4H), 2.43 (br dd, 2H), 2.30-2.15 (m, 1H), 2.04 (td, 1H), 1.34 (s, 3H).

To a solution of benzyl (S)-2-(((benzyloxy)carbonyl)amino)-4-((R,3S)-4,4,4-trifluoro-3-hydroxy-3-methylbutylsulfonimidoyl)butanoate (240 mg, 452.6 μmol, 1 eq) in THE (7 mL) was added Pd/C (1.00 g, 10% purity) and HCl (1 M, 904.71 μL, 2 eq). The mixture was stirred at 30° C. for 3 h under H2 (50 psi). The mixture was filtered and concentrated under reduced pressure. The residue was purified by prep-HPLC (column: C18-1 (150×30 mm, 5 μm); mobile phase: [water (FA)-MeCN]; gradient: 1-20% B, 10 min) to give (S)-2-amino-4-((R,3S)-4,4,4-trifluoro-3-hydroxy-3-methylbutylsulfonimidoyl)butanoic acid (Isomer 3, 60.0 mg, 169.8 μmol, 37.5% yield, FA) as white solid. LCMS: Rt=0.674 min, (ES⁺) m/z (M+H)⁺=307.0, HPLC Conditions: A. ¹H NMR (400 MHz, D₂O) δ 3.81 (t, 1H), 3.51-3.24 (m, 4H), 2.39-2.24 (m, 2H), 2.22-2.02 (m, 2H), 1.33 (s, 3H).

To a solution of benzyl (S)-2-(((benzyloxy)carbonyl)amino)-4-((S,3S)-4,4,4-trifluoro-3-hydroxy-3-methylbutylsulfonimidoyl)butanoate (320 mg, 603.4 μmol, 1 eq) in THE (8 mL) was added Pd/C (1.50 g, 10% purity, 1.00 eq) and HCl (1 M, 1.21 mL, 2 eq). The mixture was stirred at 30° C. for 3 h under H2 (50 psi). The mixture was filtered and concentrated under reduced pressure. The residue was purified by prep-HPLC (column: C18-1 (150×30 mm, 5 μm); mobile phase: [water (FA)-MeCN]; gradient: 1-10% B, 10 min) to give (S)-2-amino-4-((S,3S)-4,4,4-trifluoro-3-hydroxy-3-methylbutylsulfonimidoyl)butanoic acid (Isomer 4, 80 mg, 226.49 μmol, 37.55% yield, FA) as white solid. LCMS: Rt=0.655 min, (ES⁺) m/z (M+H)⁺=307.1, HPLC Conditions: A. ¹H NMR (400 MHz, D₂O) δ 3.87 (t, 1H), 3.54-3.32 (m, 4H), 2.45-2.29 (m, 2H), 2.28-2.06 (m, 2H), 1.38 (s, 3H).

### Exemplary Embodiment 1aa18.3

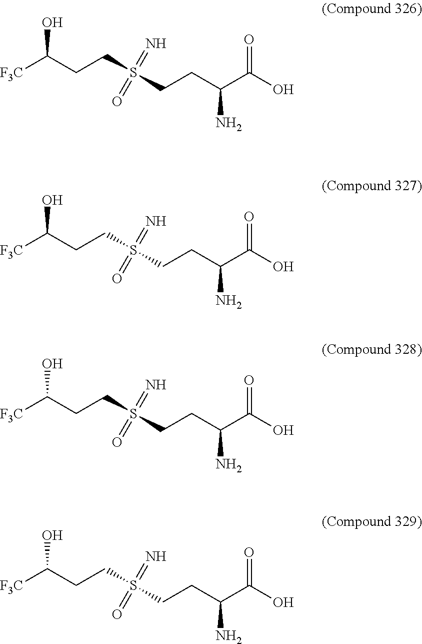 [SMILES: N=[S@@](=O)(CC[C@H](N)C(=O)O)CC[C@H](O)C(F)(F)F ; N=[S@](=O)(CC[C@H](N)C(=O)O)CC[C@H](O)C(F)(F)F ; N=[S@](=O)(CC[C@@H](O)C(F)(F)F)CC[C@H](N)C(=O)O ; N=[S@@](=O)(CC[C@@H](O)C(F)(F)F)CC[C@H](N)C(=O)O]

### Compound 326 (Isomer 1), Compound 327 (Isomer 2), Compound 328 (Isomer 3), & Compound 329 (Isomer 4)

### (S)-2-amino-4-((R,3S)-4,4,4-trifluoro-3-hydroxybutylsulfonimidoyl)butanoic acid, (S)-2-amino-4-((S,3S)-4,4,4-trifluoro-3-hydroxybutylsulfonimidoyl)butanoic acid, (S)-2-amino-4-((R,3R)-4,4,4-trifluoro-3-hydroxybutylsulfonimidoyl)butanoic acid, & (S)-2-amino-4-((S,3R)-4,4,4-trifluoro-3-hydroxybutylsulfonimidoyl)butanoic acid

### Synthesis Stage 1

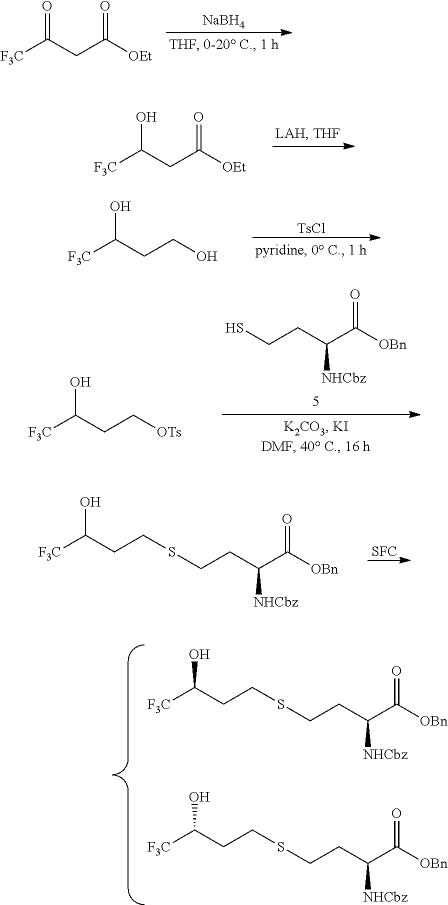

### Synthesis Stage 2

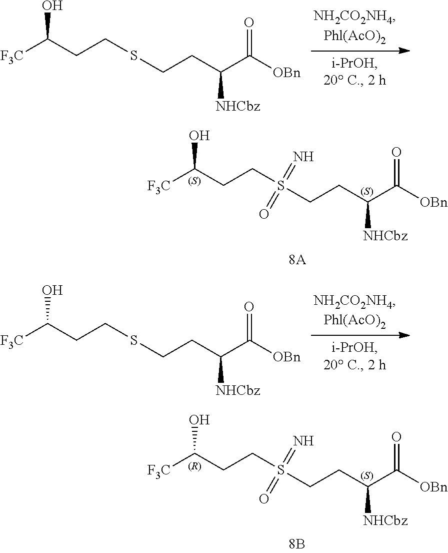

### Synthesis Stage 3

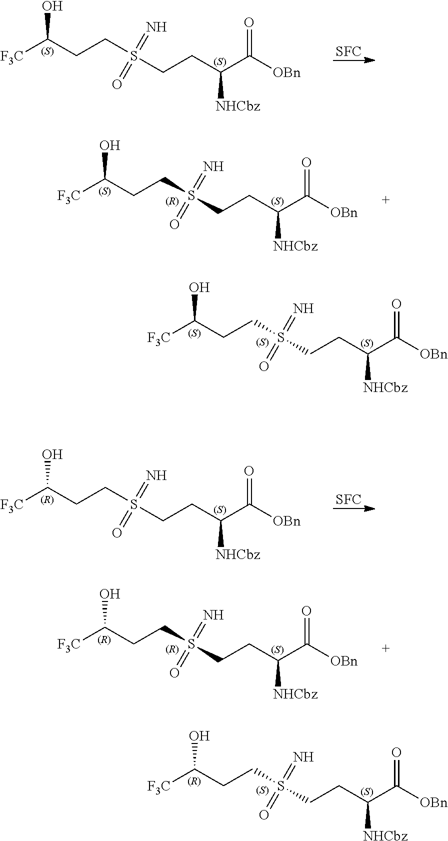

### Synthesis Stage 3

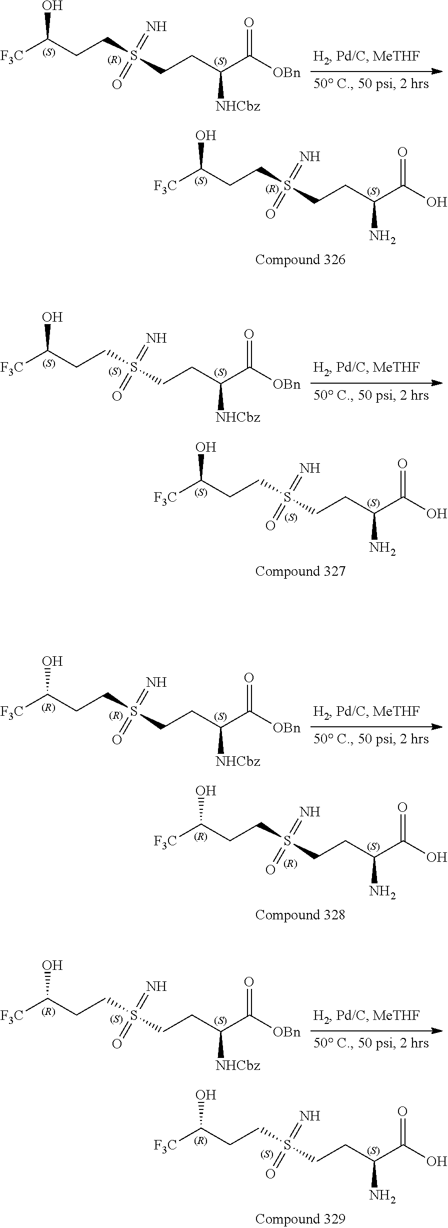

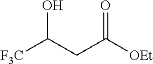 [SMILES: CCOC(=O)CC(O)C(F)(F)F]

To a mixture of ethyl 4,4,4-trifluoro-3-oxobutanoate (50 g, 271 mmol, 39.6 mL) in THE (500 mL) was added NaBH₄ (5.14 g, 135 mmol) in several portions at 0° C. under N₂. The mixture was stirred at 25° C. for 1 h. The mixture was quenched with 10% HCl solution (100 mL) and the aqueous phase was extracted with ethyl acetate (200 mL×3). The combined organic phase was washed with brine (300 mL), dried with anhydrous Na₂SO₄, filtered, and concentrated under vacuum. The residue was purified by silica gel chromatography (petroleum ether/EtOAc=30:1 to 3:1) to afford ethyl 4,4,4-trifluoro-3-hydroxybutanoate (50 g, 268 mmol, 49.4% yield) as yellow solid. ¹H NMR (400 MHz, CDCl₃-d) δ 4.47-4.44 (m, 1H), 4.27-4.22 (m, 2H), 3.43 (d, 1H), 2.74-2.70 (m, 2H), 1.32 (t, 3H).

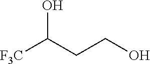

To a solution of ethyl 4,4,4-trifluoro-3-hydroxybutanoate (192.0 g, 1.03 mol) in THE (800 mL) was added LAH (18.2 g, 481 mmol) in several portions at −78° C. under N₂. The mixture was stirred at 25° C. for 1 h. The mixture was quenched with H₂O (18.2 mL) and adjusted to pH=6 with 4 N HCl solution. The aqueous phase was extracted with ethyl acetate (300 mL×5) and the combined organic phase was washed with brine (500 mL), dried with anhydrous Na₂SO₄, filtered, and concentrated under vacuum to afford crude 4,4,4-trifluorobutane-1,3-diol (148 g, 1.03 mol, 99.5% yield) as yellow solid, which was used in the next step directly. ¹H NMR (400 MHz, D₂O) δ 4.16-4.14 (m, 1H), 3.92-3.87 (m, 1H), 3.86-3.84 (m, 1H), 1.91-1.84 (m, 2H).

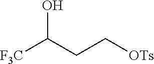 [SMILES: Cc1ccc(S(=O)(=O)OCCC(O)C(F)(F)F)cc1]

To a mixture of 4,4,4-trifluorobutane-1,3-diol (148.0 g, 1.03 mol) in pyridine (300 mL) was added TsCl (117 g, 616 mmol) in one portion at 0° C. under N₂. The mixture was stirred at 0° C. for 2 hours. The mixture was poured into ice-water (500 mL) and adjusted to pH=6 with 12 N HCl solution. The aqueous phase was extracted with ethyl acetate (200 mL×3) and the combined organic phase was washed with brine (300 mL), dried with anhydrous Na₂SO₄, filtered, and concentrated under vacuum. The residue was purified by silica gel chromatography (petroleum ether/EtOAc=30:1 to 3:1) to afford 4,4,4-trifluoro-3-hydroxybutyl 4-methylbenzenesulfonate (250 g, 838 mmol, 81.6% yield) as yellow oil. ¹H NMR (400 MHz, CDCl₃-d) δ 7.37 (d, 2H), 7.31 (d, 2H), 4.26-4.23 (m, 1H), 4.12-4.05 (m, 2H), 3.24 (d, 2H), 2.39 (s, 3H), 2.02-1.99 (m, 1H), 1.83-1.76 (m, 1H).

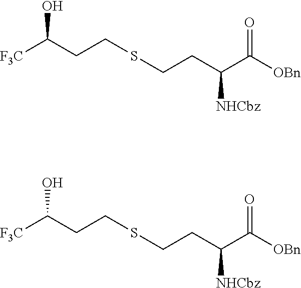 [SMILES: O=C(N[C@@H](CCSCC[C@H](O)C(F)(F)F)C(=O)OCc1ccccc1)OCc1ccccc1 ; O=C(N[C@@H](CCSCC[C@@H](O)C(F)(F)F)C(=O)OCc1ccccc1)OCc1ccccc1]

To a mixture of benzyl ((benzyloxy)carbonyl)-L-homocysteinate (60.2 g, 167 mmol) and 4,4,4-trifluoro-3-hydroxybutyl 4-methylbenzenesulfonate (50 g, 167 mmol) in DMF (600 mL) was added KI (55.6 g, 335 mmol) and K₂CO₃ (69.5 g, 502.8 mmol) in one portion at 25° C. under Ar. The mixture was stirred at 40° C. for 12 h under Argon. The mixture was poured into water (1000 mL) and the aqueous phase was extracted with ethyl acetate (300 mL×3). The combined organic phase was washed with brine (500 mL), dried with anhydrous Na₂SO₄, filtered, and concentrated under vacuum. The residue was purified by silica gel chromatography (Petroleum ether/Ethyl acetate=30:1 to 3:1) to afford benzyl N-((benzyloxy)carbonyl)-S-(4,4,4-trifluoro-3-hydroxybutyl)-L-homocysteinate (140 g). ¹H NMR (400 MHz, CDCl₃-d) δ 7.38 (m, 10H), 5.26-5.22 (m, 1H), 5.22-5.13 (m, 4H), 4.69-4.67 (m, 1H), 2.72-2.55 (m, 4H), 2.10-1.85 (m, 4H). The material was then separated by prep-SFC (column: (s,s) WHELK-01 (250×30 mm, 5 μm); mobile phase: [water (0.1% NH₃)-EtOH]; gradient: 50% B, 9 min) to afford, in order of elution, benzyl N-((benzyloxy)carbonyl)-S—((S)-4,4,4-trifluoro-3-hydroxybutyl)-L-homocysteinate (63 g, 129 mmol, 77.4% yield) and benzyl N-((benzyloxy)carbonyl)-S—((R)-4,4,4-trifluoro-3-hydroxybutyl)-L-homocysteinate (64 g, 131 mmol, 78.6% yield) as yellow solids.

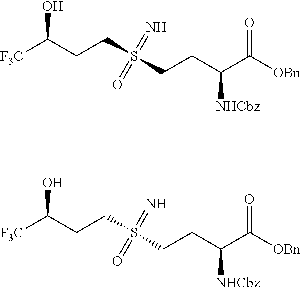 [SMILES: N=[S@@](=O)(CC[C@H](NC(=O)OCc1ccccc1)C(=O)OCc1ccccc1)CC[C@H](O)C(F)(F)F ; N=[S@](=O)(CC[C@H](NC(=O)OCc1ccccc1)C(=O)OCc1ccccc1)CC[C@H](O)C(F)(F)F]

To a solution of benzyl N-((benzyloxy)carbonyl)-S—((S)-4,4,4-trifluoro-3-hydroxybutyl)-L-homocysteinate (63 g, 130 mmol) in i-PrOH (330 mL) was added [acetoxy(phenyl)-k3-iodanyl]acetate (55.7 g, 173 mmol) and ammonium carbamate (27.0 g, 346 mmol) in one portion at 25° C. The mixture was stirred at 25° C. for 2 hours. The mixture was poured into water (500 mL) and the aqueous phase was extracted with ethyl acetate (300 mL×3). The combined organic phase was washed with brine (500 mL), dried with anhydrous Na₂SO₄, filtered, and concentrated under vacuum. The residue was purified by silica gel chromatography (petroleum ether/EtOAc=20:1 to 0:1) to afford crude product benzyl (2S)-2-(((benzyloxy)carbonyl)amino)-4-((3S)-4,4,4-trifluoro-3-hydroxybutylsulfonimidoyl)butanoate, which was then separated by prep-SFC (column: Daicel CHIRALPAK AD (250×50 mm, 10 μm); mobile phase: [water (0.1% NH₃)— MeOH]; gradient: 30% B). Benzyl (S)-2-(((benzyloxy)carbonyl)amino)-4-((R,3S)-4,4,4-trifluoro-3-hydroxybutylsulfonimidoyl)butanoate (14 g, 27.1 mmol, 20.8% yield) was obtained as white solid. ¹H NMR (400 MHz, CDCl₃-d) δ 7.39-7.35 (m, 10H), 5.68 (d, 1H), 5.23-5.12 (m, 4H), 4.56-4.54 (m, 1H), 4.24-4.21 (m, 1H), 3.31-3.00 (m, 4H), 2.31-2.11 (m, 4H). benzyl (S)-2-(((benzyloxy)carbonyl)amino)-4-((S,3S)-4,4,4-trifluoro-3-hydroxybutylsulfonimidoyl)butanoate (21 g, 40.6 mmol, 31.3% yield) was obtained as white solid. ¹H NMR (400 MHz, CDCl₃-d) δ 7.30-7.25 (m, 10H), 5.63 (d, 1H), 5.17-5.02 (m, 4H), 4.45-4.44 (m, 1H), 4.03-3.96 (m, 1H), 3.09-2.95 (m, 4H), 2.34-2.33 (m, 1H), 2.16-2.11 (m, 3H).

To a solution of benzyl (S)-2-(((benzyloxy)carbonyl)amino)-4-((R,3S)-4,4,4-trifluoro-3-hydroxybutylsulfonimidoyl)butanoate (14.0 g, 27.1 mmol) in MeTHF (70 mL) and HCl (0.5 M, 15.34 mL) was added 10% Pd/C (4.66 g) under N₂. The suspension was degassed under vacuum and purged with H₂ several times. The mixture was stirred under H₂ (50 psi) at 30° C. for 2 hours. The reaction mixture was filtered, and the filtrate was concentrated and lyophilized. The resultant solid was washed with MTBE (200 mL) and filtered to give (S)-2-amino-4-((R,3S)-4,4,4-trifluoro-3-hydroxybutylsulfonimidoyl)butanoic acid (Isomer 1, 8.1 g, 24.6 mmol, 91.0% yield, HCl) as white solid. LCMS: Rt=0.418 min, (ES⁺) m/z (M+H)⁺=293.0, HPLC Conditions: A. ¹H NMR (400 MHz, D₂O) δ 4.22-4.19 (m, 1H), 3.98-3.95 (m, 1H), 3.76-3.51 (m, 4H), 2.39-2.36 (m, 2H), 2.27-2.24 (m, 1H), 2.10-2.06 (m, 1H).

To a solution of benzyl (S)-2-(((benzyloxy)carbonyl)amino)-4-((S,3S)-4,4,4-trifluoro-3-hydroxybutylsulfonimidoyl)butanoate (16 g, 31.0 mmol) in MeTHF (60 mL) and HCl (0.5 M, 13.16 mL) was added 10% Pd/C (4 g) under N₂. The suspension was degassed under vacuum and purged with H₂ several times. The mixture was stirred under H₂ (50 psi) at 30° C. for 2 hours. The reaction mixture was filtered, and the filtrate was concentrated and lyophilized. The resultant solid was washed with MTBE (200 mL) and filtered to give (S)-2-amino-4-((S,3S)-4,4,4-trifluoro-3-hydroxybutylsulfonimidoyl)butanoic acid (Isomer 2, 9.12 g, 27.7 mmol, 89.5% yield, HCl) as yellow solid. LCMS: Rt=0.427 min, (ES⁺) m/z (M+H)⁺=293.0, HPLC Conditions: A. ¹H NMR (400 MHz, D₂O) δ 4.22-4.18 (m, 1H), 3.93-3.90 (m, 1H), 3.57-3.51 (m, 4H), 2.36-2.34 (m, 2H), 2.26-2.23 (m, 1H), 2.09-2.07 (m, 1H).

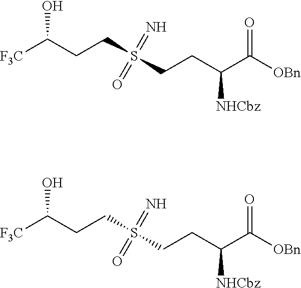 [SMILES: N=[S@@](=O)(CC[C@H](NC(=O)OCc1ccccc1)C(=O)OCc1ccccc1)CC[C@@H](O)C(F)(F)F ; N=[S@](=O)(CC[C@H](NC(=O)OCc1ccccc1)C(=O)OCc1ccccc1)CC[C@@H](O)C(F)(F)F]

To a solution of benzyl N-((benzyloxy)carbonyl)-S—((R)-4,4,4-trifluoro-3-hydroxybutyl)-L-homocysteinate (68.4 g, 141 mmol) in i-PrOH (330 mL) was added [acetoxy(phenyl)-λ3-iodanyl]acetate (60.5 g, 187 mmol) and ammonium carbamate (29.3 g, 375 mmol) in one portion at 25° C. The mixture was stirred at 25° C. for 2 hours. The mixture was poured into water (500 mL) and the aqueous phase was extracted with ethyl acetate (300 mL×3). The combined organic phase was washed with brine (500 mL), dried with anhydrous Na₂SO₄, filtered, and concentrated under vacuum. The residue was purified by silica gel chromatography (Petroleum ether/Ethyl acetate=20:1 to 0:1) to afford crude benzyl (2S)-2-(((benzyloxy)carbonyl)amino)-4-((3R)-4,4,4-trifluoro-3-hydroxybutylsulfonimidoyl)butanoate, which was then separated by prep-SFC (column: Daicel CHIRALPAK AD (250×50 mm, 10 μm); mobile phase: [water (0.1% NH₃)-MeOH]; gradient: 25% B). Benzyl (S)-2-(((benzyloxy)carbonyl)amino)-4-((R,3R)-4,4,4-trifluoro-3-hydroxybutylsulfonimidoyl)butanoate (19.5 g, 37.7 mmol, 26.7% yield) was obtained as a white solid. ¹H NMR (400 MHz, CDCl₃-d) δ 7.29-7.27 (m, 10H), 5.68 (d, 1H), 5.23-5.02 (m, 4H), 4.46-4.45 (m, 1H), 4.01-3.96 (m, 1H), 3.23-2.76 (m, 4H), 2.38-2.32 (m, 1H), 2.15-2.07 (m, 3H). Benzyl (S)-2-(((benzyloxy)carbonyl)amino)-4-((S,3R)-4,4,4-trifluoro-3-hydroxybutylsulfonimidoyl)butanoate (14 g, 27.1 mmol, 19.2% yield) was obtained as white solid. ¹H NMR (400 MHz, CDCl₃-d) δ 7.29-7.28 (m, 10H), 5.57 (d, 1H), 5.18-5.03 (m, 4H), 4.47-4.45 (m, 1H), 4.17-4.13 (m, 1H), 3.29-3.24 (m, 1H), 3.08-2.96 (m, 3H), 2.17-2.02 (m, 4H).

To a solution of benzyl (S)-2-(((benzyloxy)carbonyl)amino)-4-((R,3R)-4,4,4-trifluoro-3-hydroxybutylsulfonimidoyl)butanoate (19.5 g, 37.7 mmol) in MeTHF (70 mL) and HCl (0.5 M, 16.0 mL) was added 10% Pd/C (4.88 g) under N₂. The suspension was degassed under vacuum and purged with H₂ several times. The mixture was stirred under H₂ (50 psi) at 30° C. for 2 hours. The reaction mixture was filtered, and the filtrate was concentrated and lyophilized. The resultant solid was washed with MTBE (200 mL) and filtered to give (S)-2-amino-4-((R,3R)-4,4,4-trifluoro-3-hydroxybutylsulfonimidoyl)butanoic acid (Isomer 3, 9.05 g, 27.5 mmol, 72.9% yield, HCl) as white solid. LCMS: Rt=0.418 min, (ES⁺) m/z (M+H)⁺=293.0, HPLC Conditions: A. ¹H NMR (400 MHz, D₂O) δ 4.22-4.19 (m, 1H), 3.91-3.88 (m, 1H), 3.54-3.49 (m, 4H), 2.36-2.34 (m, 2H), 2.25-2.22 (m, 1H), 2.08-2.05 (m, 1H).

To a solution of benzyl (S)-2-(((benzyloxy)carbonyl)amino)-4-((S,3R)-4,4,4-trifluoro-3-hydroxybutylsulfonimidoyl)butanoate (18 g, 34.8 mmol) in MeTHF (70 mL) and HCl (0.5 M, 14.8 mL) was added 10% Pd/C (4.5 g) under N₂. The suspension was degassed under vacuum and purged with H₂ several times. The mixture was stirred under H₂ (50 psi) at 30° C. for 2 hours. The reaction mixture was filtered, and the filtrate was concentrated and lyophilized. The resultant solid was washed with MTBE (200 mL) and filtered to give (S)-2-amino-4-((S,3R)-4,4,4-trifluoro-3-hydroxybutylsulfonimidoyl)butanoic acid (Isomer 4, 7.04 g, 21.4 mmol, 61.5% yield, HCl) as white solid. LCMS: Rt=0.404 min, (ES⁺) m/z (M+H)⁺=293.0, HPLC Conditions: A. ¹H NMR (400 MHz, D₂O) δ 4.21-4.20 (m, 1H), 3.97-3.96 (m, 1H), 3.65-3.58 (m, 4H), 2.38-2.37 (m, 2H), 2.25-2.22 (m, 1H), 2.09-2.06 (m, 1H).

The compounds described in Table 12 were prepared using the general methods outlined above.

TABLE 12
Characterization of Compounds 293-329

| Compound | | MS | HPLC | HPLC |
|---|---|---|---|---|
| # | Structure | (M + H)⁺ | Rt | cond. |
| 293 | 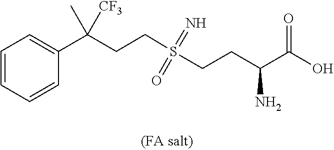 [SMILES: CC(CCS(=N)(=O)CC[C@H](N)C(=O)O)(c1ccccc1)C(F)(F)F] | 367.1 | 1.860 | C |
| | ¹H NMR (400 MHz, D₂O) δ 7.48 (br d, 2H), 7.43- | | | |
| | 7.30 (m, 3H), 3.73 (q, 1H), 3.39-3.02 (m, 3H), | | | |
| | 2.83-2.60 (m, 2H), 2.32-2.10 (m, 3H), 1.54 (s, | | | |
| | 3H) | | | |
| 294 | 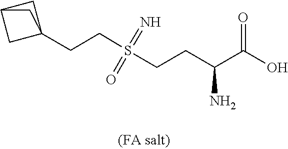 [SMILES: N=S(=O)(CC[C@H](N)C(=O)O)CCC12CC(C1)C2] | 261.2 | 1.77 | T |
| | ¹H NMR (400 MHz, D₂O) δ 3.84-3.76 (m, 1 H), | | | |
| | 3.38-3.12 (m, 4 H), 2.41 (s, 1 H), 2.34-2.21 (m, 2 | | | |
| | H), 1.91-1.80 (m, 2 H), 1.63 (s, 6 H) | | | |
| 295 | 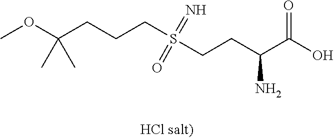 [SMILES: COC(C)(C)CCCS(=N)(=O)CC[C@H](N)C(=O)O] | 281.2 | 0.686 | T |
| | ¹H NMR (400 MHz, D₂O) δ 3.86 (br d, 1H), 3.47- | | | |
| | 3.24 (m, 4H), 3.17 (s, 3H), 2.40-2.28 (m, 2H), | | | |
| | 1.78 (br d, 2H), 1.68-1.59 (m, 2H), 1.18 (s, 6H) | | | |
| 296 | 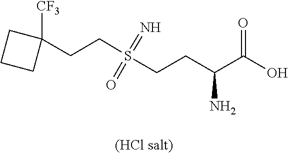 [SMILES: N=S(=O)(CC[C@H](N)C(=O)O)CCC1(C(F)(F)F)CCC1] | 317.1 | 1.627 | A |
| | ¹H NMR (400 MHz, D₂O) δ 4.16-4.08 (m, 1H), | | | |
| | 4.01-3.64 (m, 4H), 2.56-2.43 (m, 2H), 2.39-2.26 | | | |
| | (m, 4H), 2.00-1.90 (m, 4H) | | | |
| 297 | 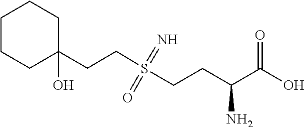 [SMILES: N=S(=O)(CC[C@H](N)C(=O)O)CCC1(O)CCCCC1] | 293.2 | 0.874 | T |
| | ¹H NMR (400 MHz, D₂O) δ 3.86-3.68 (m, 2H), | | | |
| | 3.39 (br d, 1H), 3.36-3.26 (m, 2H), 2.32-2.22 (m, | | | |
| | 2H), 1.99-1.85 (m, 2H), 1.63-1.62 (m, 1H), 1.70- | | | |
| | 1.25 (m, 9H) | | | |
| 298 | 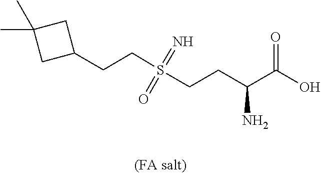 [SMILES: CC1(C)CC(CCS(=N)(=O)CC[C@H](N)C(=O)O)C1] | 293.2 | 0.874 | T |
| | ¹H NMR (400 MHz, D₂O) δ 3.90-3.83 (m, 1H), | | | |
| | 3.48-3.24 (m, 2H), 3.18-3.10 (m, 2H), 2.38-2.23 | | | |
| | (m, 3H), 1.91-1.81 (m, 4H), 1.49-1.39 (m, 2H), | | | |
| | 1.10 (s, 3H), 1.02 (s, 3H) | | | |
| 299 | 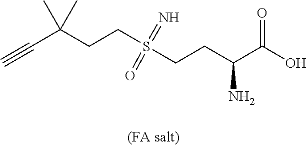 [SMILES: C#CC(C)(C)CCS(=N)(=O)CC[C@H](N)C(=O)O] | 261.1 | 1.798 | A |
| | ¹H NMR (400 MHz, D₂O) δ 3.88 (dt, 1H), 3.57- | | | |
| | 3.36 (m, 4H), 2.59 (s, 1H), 2.44-2.30 (m, 2H), | | | |
| | 2.01-1.81 (m, 2H), 1.25 (s, 6H) | | | |
| 300 | 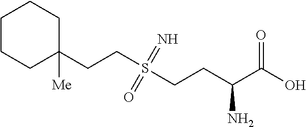 [SMILES: CC1(CCS(=N)(=O)CC[C@H](N)C(=O)O)CCCCC1] | 291.1 | 1.798 | C |
| | ¹H NMR (400 MHz, D₂O) δ 3.85-3.77 (m, 1H), | | | |
| | 3.45-3.11 (m, 4H), 2.34-2.23 (m, 2H), 1.71-1.58 | | | |
| | (m, 2H), 1.42-1.15 (m, 10H), 0.82 (s, 3H) | | | |
| 301 | 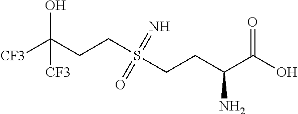 [SMILES: N=S(=O)(CC[C@H](N)C(=O)O)CCC(O)(C(F)(F)F)C(F)(F)F] | 361.1 | 1.048 | C |
| | ¹H NMR (400 MHz, D₂O) δ 3.87 (dt, 1H), 3.56- | | | |
| | 3.50 (m, 2H), 3.50-3.31 (m, 2H), 2.55-2.46 (m, | | | |
| | 2H), 2.36 (td, 2H) | | | |
| 302 | 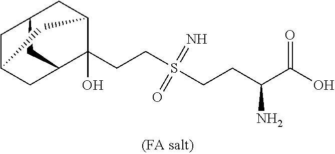 [SMILES: N=S(=O)(CC[C@H](N)C(=O)O)CCC1(O)[C@H]2C[C@@H]3C[C@@H](C[C@H]1C3)C2] | 345.1 | 1.744 | C |
| | ¹H NMR (400 MHz, D₂O) δ 8.37 (s, 1H), 3.82 (dt, | | | |
| | 1H), 3.47-3.17 (m, 4H), 2.37-2.23 (m, 2H), 2.16- | | | |
| | 1.97 (m, 4H), 1.84-1.59 (m, 10H), 1.53 (br d, | | | |
| | 2H) | | | |
| 303 | 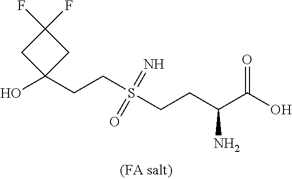 [SMILES: N=S(=O)(CC[C@H](N)C(=O)O)CCC1(O)CC(F)(F)C1] | 301.0 | 0.541 | C |
| | ¹H NMR (400 MHz, D₂O) δ 3.88 (dt, 1H), 3.59- | | | |
| | 3.33 (m, 4H), 2.87-2.60 (m, 4H), 2.44-2.31 (m, | | | |
| | 2H), 2.20 (t, 2H) | | | |
| 304 | 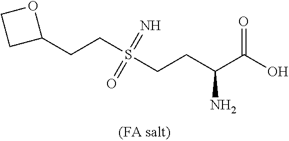 [SMILES: N=S(=O)(CCC1CCO1)CC[C@H](N)C(=O)O] | 251.1 | 0.503 | G |
| | ¹H NMR (400 MHz, D₂O) δ 4.95 (br t, 1H), 4.67- | | | |
| | 4.61 (m, 1H), 4.52 (td, 1H), 3.53-3.33 (m, 1H), | | | |
| | 3.30-3.10 (m, 4H), 2.77-2.66 (m, 1H), 2.46-2.33 | | | |
| | (m, 1H), 2.26-1.92 (m, 4H). | | | |
| 305 | 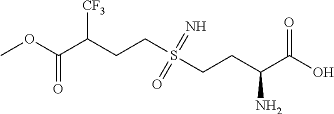 [SMILES: COC(=O)C(CCS(=N)(=O)CC[C@H](N)C(=O)O)C(F)(F)F] | 335.0 | 0.804 | C |
| | ¹H NMR (400 MHz, D₂O) δ 3.85-3.79 (m, 1H), | | | |
| | 3.76 (s, 3H), 3.71-3.62 (m, 1H), 3.46-3.27 (m, | | | |
| | 4H), 2.38-2.25 (m, 4H). | | | |
| 306 | 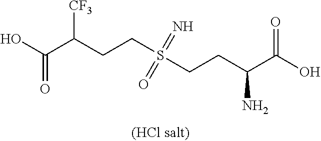 [SMILES: N=S(=O)(CCC(C(=O)O)C(F)(F)F)CC[C@H](N)C(=O)O] | 321.0 | 0.367 | C |
| | ¹H NMR (400 MHz, D₂O) δ 3.87-3.78 (m, 1H), | | | |
| | 3.49-3.20 (m, 4H), 3.14 (td, 1H), 2.40-2.25 (m, | | | |
| | 2H), 2.19 (q, 2H). | | | |
| 307 | 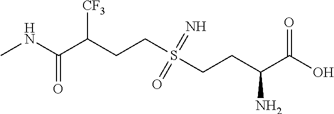 [SMILES: CNC(=O)C(CCS(=N)(=O)CC[C@H](N)C(=O)O)C(F)(F)F] | 334.1 | 0.394 | A |
| | ¹H NMR (400 MHz, D₂O) δ 3.98 (dt, 1H), 3.70- | | | |
| | 3.32 (m, 5H), 2.82-2.65 (m, 3H), 2.44-2.20 (m, | | | |
| | 4H). | | | |
| 308 | 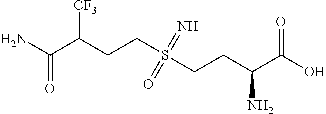 [SMILES: N=S(=O)(CCC(C(N)=O)C(F)(F)F)CC[C@H](N)C(=O)O] | 320.1 | 0.440 | A |
| | ¹H NMR (400 MHz, D₂O) δ 4.06-3.94 (m, 1H), | | | |
| | 3.77-3.36 (m, 5H), 2.51-2.22 (m, 4H). | | | |
| 309 | 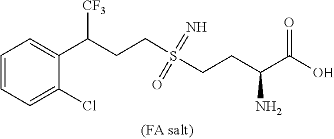 [SMILES: N=S(=O)(CCC(c1ccccc1Cl)C(F)(F)F)CC[C@H](N)C(=O)O] | 386.9 | 3.77 | E |
| | ¹H NMR (400 MHz, D₂O) δ 7.48 (br dd, 2H), 7.38- | | | |
| | 7.23 (m, 2H), 4.46-4.25 (m, 1H), 3.72 (br d, 1H), | | | |
| | 3.40-3.10 (m, 3H), 3.00-2.82 (m, 1H), 2.59-2.44 | | | |
| | (m, 1H), 2.41-2.25 (m, 1H), 2.17 (br s, 2H) | | | |
| 310 | 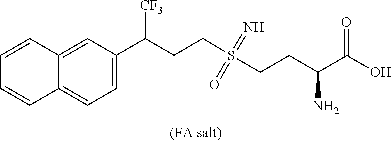 [SMILES: N=S(=O)(CCC(c1ccc2ccccc2c1)C(F)(F)F)CC[C@H](N)C(=O)O] | 403.1 | 2.11 | C |
| | ¹H NMR (400 MHz, D₂O) δ 7.88-8.05 (m, 4 H) | | | |
| | 7.50-7.66 (m, 3 H) 3.82-3.97 (m, 1 H) 3.72-3.81 | | | |
| | (m, 1 H) 3.16-3.45 (m, 3 H) 2.97 (br d, 1 H) 2.44- | | | |
| | 2.71 (m, 2 H) 2.22 (br s, 2 H) | | | |
| 311 | 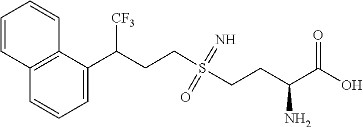 [SMILES: N=S(=O)(CCC(c1cccc2ccccc12)C(F)(F)F)CC[C@H](N)C(=O)O] | 403.1 | 2.09 | C |
| | ¹H NMR (400 MHz, D₂O) δ 8.20 (s, 1H), 8.02 (br | | | |
| | d, 2H), 7.77 (br d, 1H), 7.71-7.57 (m, 3H), 3.84- | | | |
| | 3.70 (m, 1H), 3.39-3.17 (m, 3H), 3.06-2.89 (m, | | | |
| | 1H), 2.82-2.59 (m, 2H), 2.28-2.14 (m, 2H) | | | |
| 312 | 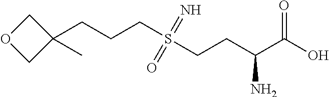 [SMILES: CC1(CCCS(=N)(=O)CC[C@H](N)C(=O)O)COC1] | 279.1 | 0.359 | C |
| | ¹H NMR (400 MHz, D₂O) δ 4.50 (d, 2H), 4.42 (d, | | | |
| | 2H), 3.90-3.81 (m, 1H), 3.47-3.24 (m, 4H), 2.39- | | | |
| | 2.26 (m, 2H), 1.78 (br d, 4H), 1.29 (s, 3H) | | | |
| 313 | 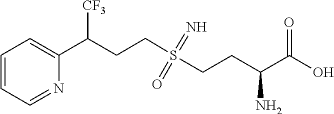 [SMILES: N=S(=O)(CCC(c1ccccn1)C(F)(F)F)CC[C@H](N)C(=O)O] | 354.1 | 1.31 | C |
| | ¹H NMR (400 MHz, D₂O) δ 8.55 (d, 1H), 7.92 (dt, | | | |
| | 1H), 7.58-7.44 (m, 2H), 4.04-3.76 (m, 2H), 3.48- | | | |
| | 3.19 (m, 3H), 3.09-2.94 (m, 1H), 2.57 (dq, 2H), | | | |
| | 2.37-2.19 (m, 2H) | | | |
| 314 | 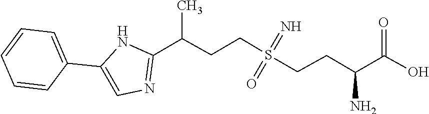 [SMILES: CC(CCS(=N)(=O)CC[C@H](N)C(=O)O)c1ncc(-c2ccccc2)[nH]1] | 419.2. | 2.05 | B |
| | ¹H NMR (400 MHz, D₂O) δ 7.65 (br d, 2H), 7.50- | | | |
| | 7.37 (m, 3H), 7.33-7.27 (m, 1H), 4.07-3.95 (m, | | | |
| | 1H), 3.80-3.71 (m, 1H), 3.44-3.22 (m, 3H), 3.15- | | | |
| | 3.03 (m, 1H), 2.60-2.48 (m, 2H), 2.24 (br d, 2H) | | | |
| 315 | 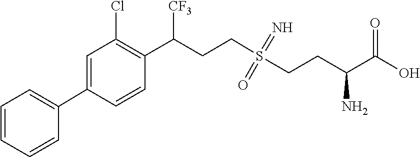 [SMILES: N=S(=O)(CCC(c1ccc(-c2ccccc2)cc1Cl)C(F)(F)F)CC[C@H](N)C(=O)O] | 463.1 | 2.25 | C |
| | ¹H NMR (400 MHz, MeOD-d₄) δ 7.77 (d, 1H), 7.71- | | | |
| | 7.64 (m, 4H), 7.50-7.45 (m, 2H), 7.40 (d, 1H), | | | |
| | 4.48-4.33 (m, 1H), 3.70-3.59 (m, 1H), 3.42-3.35 | | | |
| | (m, 1H), 3.27-3.15 (m, 2H), 2.97-2.80 (m, 1H), | | | |
| | 2.70-2.57 (m, 1H), 2.56-2.41 (m, 1H), 2.34-2.22 | | | |
| | (m, 2H) | | | |
| 316 | 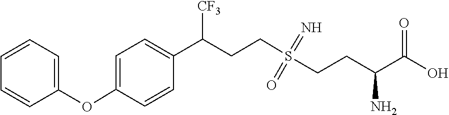 [SMILES: N=S(=O)(CCC(c1ccc(Oc2ccccc2)cc1)C(F)(F)F)CC[C@H](N)C(=O)O] | 445.1 | 2.46 | B |
| | ¹H NMR (400 MHz, MeOD-d₄) δ 7.43-7.37 (m, | | | |
| | 4H), 7.20-7.14 (m, 1H), 7.04 (t, 4H), 3.78-3.64 | | | |
| | (m, 2H), 3.45-3.35 (m, 1H), 3.31-3.21 (m, 1H), | | | |
| | 3.20-3.09 (m, 1H), 2.99-2.85 (m, 1H), 2.64-2.39 | | | |
| | (m, 2H), 2.34-2.23 (m, 2H) | | | |
| 317 | 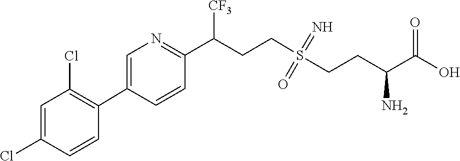 [SMILES: N=S(=O)(CCC(c1ccc(-c2ccc(Cl)cc2Cl)cn1)C(F)(F)F)CC[C@H](N)C(=O)O] | 498.1 | 2.18 | C |
| | ¹H NMR (400 MHz, MeOD-d₄) δ 8.69 (d, 1H), 7.97 | | | |
| | (dd, 1H), 7.65 (d, 1H), 7.60 (d, 1H), 7.50-7.44 (m, | | | |
| | 2H), 4.11-3.96 (m, 1H), 3.77-3.67 (m, 1H), 3.39- | | | |
| | 3.32 (m, 2H), 3.25-3.11 (m, 1H), 3.08-2.93 (m, | | | |
| | 1H), 2.82-2.68 (m, 1H), 2.65-2.51 (m, 1H), 2.39- | | | |
| | 2.25 (m, 2H) | | | |
| 318 | 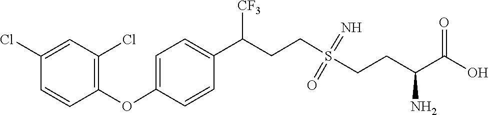 [SMILES: N=S(=O)(CCC(c1ccc(Oc2ccc(Cl)cc2Cl)cc1)C(F)(F)F)CC[C@H](N)C(=O)O] | 513.1 | 2.32 | C |
| | ¹H NMR (400 MHz, MeOD-d₄) δ 7.59 (d, 1H), 7.40 | | | |
| | (d, 2H), 7.35 (dd, 1H), 7.08 (d, 1H), 6.98 (d, 2H), | | | |
| | 3.76-3.64 (m, 2H), 3.43-3.34 (m, 1H), 3.29-3.20 | | | |
| | (m, 1H), 3.19-3.07 (m, 1H), 2.96-2.82 (m, 1H), | | | |
| | 2.63-2.49 (m, 1H), 2.49-2.35 (m, 1H), 2.34-2.22 | | | |
| | (m, 2H) | | | |
| 319 | 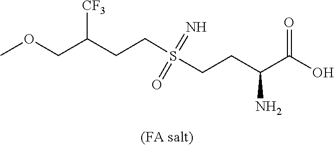 [SMILES: COCC(CCS(=N)(=O)CC[C@H](N)C(=O)O)C(F)(F)F] | 321.0 | 0.910 | C |
| | ¹H NMR (400 MHz, D₂O) δ 3.87-3.76 (m, 1H), | | | |
| | 3.70-3.55 (m, 2H), 3.51-3.22 (m, 7H), 2.78-2.58 | | | |
| | (m, 1H), 2.37-2.23 (m, 2H), 2.21-1.94 (m, 2H). | | | |
| 320 | 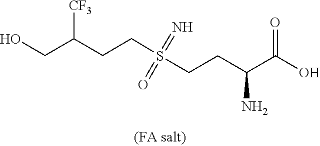 [SMILES: N=S(=O)(CCC(CO)C(F)(F)F)CC[C@H](N)C(=O)O] | 307.0 | 0.415 | E |
| | ¹H NMR (400 MHz, D₂O) δ 3.90-3.84 (m, 1H), | | | |
| | 3.83-3.76 (m, 2H), 3.56-3.25 (m, 4H), 2.68-2.49 | | | |
| | (m, 1H), 2.35 (q, 2H), 2.24-2.00 (m, 2H). | | | |
| 321 | 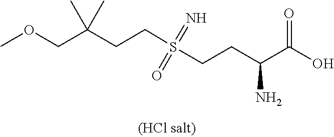 [SMILES: COCC(C)(C)CCS(=N)(=O)CC[C@H](N)C(=O)O] | 281.1 | 0.895 | C |
| | ¹H NMR (400 MHz, D₂O) δ 3.96 (br t, 1H), 3.83- | | | |
| | 3.62 (m, 2H), 3.59-3.47 (m, 2H), 3.33 (s, 3H), | | | |
| | 3.22 (s, 2H), 2.41 (q, 2H), 1.78 (t, 2H), 0.92 (s, 6H). | | | |
| 322 | 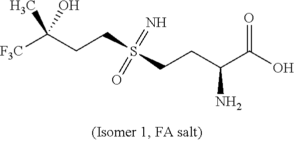 | 307.0 | 0.69 | A |
| | ¹H NMR (400 MHz, D₂O) δ 3.86 (t, 1H), 3.52- | | | |
| | 3.31 (m, 4H), 2.41-2.31 (m, 2H), 2.27-2.09 (m, | | | |
| | 2H), 1.38 (s, 3H). | | | |
| 323 | 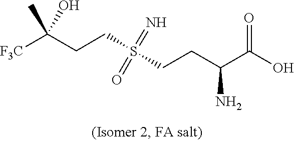 | 307.1 | 0.64 | A |
| | ¹H NMR (400 MHz, D₂O) δ 3.86 (t, 1H), 3.52- | | | |
| | 3.31 (m, 4H), 2.41-2.31 (m, 2H), 2.27-2.09 (m, | | | |
| | 2H), 1.38 (s, 3H) | | | |
| 324 | 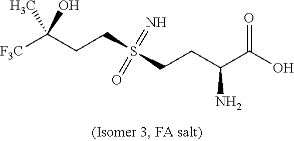 | 307.0 | 0.67 | A |
| | ¹H NMR (400 MHz, D₂O) δ 3.81 (t, 1H), 3.51- | | | |
| | 3.24 (m, 4H), 2.39-2.24 (m, 2H), 2.22-2.02 (m, | | | |
| | 2H), 1.33 (s, 3H) | | | |
| 325 | 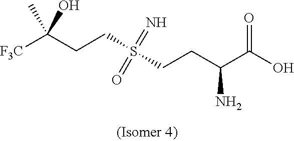 | 307.1 | 0.65 | A |
| | ¹H NMR (400 MHz, D₂O) δ 3.87 (t, 1H), 3.54- | | | |
| | 3.32 (m, 4H), 2.45-2.29 (m, 2H), 2.28-2.06 (m, | | | |
| | 2H), 1.38 (s, 3H) | | | |
| 326 | 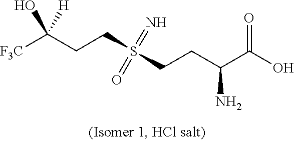 | 293.0 | 0.418 | A |
| | ¹H NMR (400 MHz, D₂O) δ 4.22-4.19 (m, 1H), | | | |
| | 3.98-3.95 (m, 1H), 3.76-3.51 (m, 4H), 2.39-2.36 | | | |
| | (m, 2H), 2.27-2.24 (m, 1H), 2.10-2.06 (m, 1H) | | | |
| 327 | 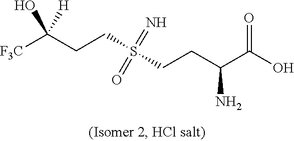 | 293.0 | 0.427 | A |
| | ¹H NMR (400 MHz, D₂O) δ 4.22-4.18 (m, 1H), | | | |
| | 3.93-3.90 (m, 1H), 3.57-3.51 (m, 4H), 2.36-2.34 | | | |
| | (m, 2H), 2.26-2.23 (m, 1H), 2.09-2.07 (m, 1H) | | | |
| 328 | 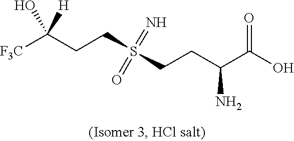 | 293.0 | 0.418 | A |
| | ¹H NMR (400 MHz, D₂O) δ 4.22-4.19 (m, 1H), | | | |
| | 3.91-3.88 (m, 1H), 3.54-3.49 (m, 4H), 2.36-2.34 | | | |
| | (m, 2H), 2.25-2.22 (m, 1H), 2.08-2.05 (m, 1H) | | | |
| 329 | 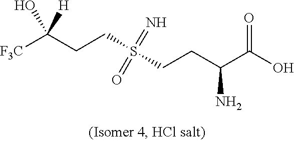 | 293.0 | 0.404 | A |
| | ¹H NMR (400 MHz, D₂O) δ 4.21-4.20 (m, 1H), | | | |
| | 3.97-3.96 (m, 1H), 3.65-3.58 (m, 4H), 2.38-2.37 | | | |
| | (m, 2H), 2.25-2.22 (m, 1H), 2.09-2.06 (m, 1H) | | | |

### Exemplary Embodiment 1aa19

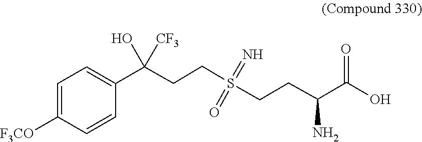 [SMILES: N=S(=O)(CC[C@H](N)C(=O)O)CCC(O)(c1ccc(OC(F)(F)F)cc1)C(F)(F)F]

### (2S)-2-amino-4-(4,4,4-trifluoro-3-hydroxy-3-(4-(trifluoromethoxy)phenyl)butylsulfonimidoyl)butanoic acid

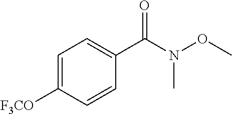 [SMILES: CON(C)C(=O)c1ccc(OC(F)(F)F)cc1]

To a solution of 4-(trifluoromethoxy)benzoic acid (2 g, 9.7 mmol, 1 eq), N,O-dimethylhydroxylamine hydrochloride (1.42 g, 14.5 mmol, 1.5 eq), and TEA (2.95 g, 29.1 mmol, 4.05 mL, 3 eq) in DCM (40 mL) was added T₃P (12.3 g, 19.4 mmol, 194 μL, 50% purity, 2 eq) at 15° C. under N₂. The mixture was stirred at 15° C. for 16 h and then concentrated under reduced pressure. The reaction mixture was poured into water (35 mL) and extracted with DCM (50 mL×2). The combined organic layers were washed with brine (20 mL), dried over Na₂SO₄, filtered, and concentrated under reduced pressure to give a residue. The residue was purified by column chromatography (SiO₂, petroleum ether/EtOAc=90:10) to give N-methoxy-N-methyl-4-(trifluoromethoxy)benzamide (2.22 g, 8.91 mmol, 91.8% yield) as white oil. ¹H NMR (400 MHz, CDCl₃-d) δ 7.83-7.77 (m, 2H), 7.27 (s, 2H), 3.58 (s, 3H), 3.41 (s, 3H).

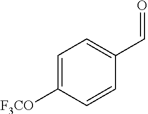 [SMILES: O=Cc1ccc(OC(F)(F)F)cc1]

To a solution of N-methoxy-N-methyl-4-(trifluoromethoxy)benzamide (1.6 g, 6.42 mmol, 1 eq) in THF (16 mL) was added dropwise LAH (2.5 M, 3.08 mL, 1.2 eq) at 0° C. under N₂. The mixture was stirred at 15° C. for 2 h. The mixture was quenched with water (0.3 mL), 15% aqueous NaOH (0.3 mL), and water (0.9 mL) at 0° C. The mixture was filtered, washed with EtOAc (20 mL), and the filtrate was concentrated to give crude 4-(trifluoromethoxy)benzaldehyde (1.2 g) as yellow oil. 1H NMR (400 MHz, CDCl₃-d) δ 10.03 (s, 1H), 7.99-7.93 (m, 2H), 7.38 (d, 2H).

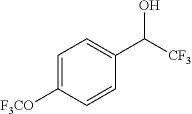 [SMILES: OC(c1ccc(OC(F)(F)F)cc1)C(F)(F)F]

To a solution of 4-(trifluoromethoxy)benzaldehyde (0.425 g, 2.24 mmol, 319 μL, 1 eq) in THE (5 mL) was added TMSCF₃ (1.59 g, 11 mmol, 5 eq) and TBAF (1 M, 223 μL, 0.1 eq) at 0° C. under N₂. The mixture was stirred at 15° C. for 0.5 h. Additional TBAF (1 M, 6.71 mL, 3 eq) was added dropwise at 0° C. The mixture was stirred at 15° C. for 2 h. The reaction mixture was poured into water (35 mL) and extracted with EtOAc (20 mL×2). The combined organic layers were washed with brine (30 mL), dried over Na₂SO₄, filtered, and concentrated under reduced pressure. The residue was purified by column chromatography (SiO₂, petroleum ether/EtOAc=3:1) to give 2,2,2-trifluoro-1-(4-(trifluoromethoxy)phenyl)ethan-1-ole (0.7 g, 2.69 mmol, 60.1% yield) as yellow oil. ¹H NMR (400 MHz, CDCl₃-d) δ 7.53 (d, 2H), 7.25 (d, 2H), 5.10-5.01 (m, 1H), 2.70 (d, 1H).

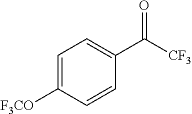 [SMILES: O=C(c1ccc(OC(F)(F)F)cc1)C(F)(F)F]

To a solution of 2,2,2-trifluoro-1-(4-(trifluoromethoxy)phenyl)ethan-1-ole (0.7 g, 2.69 mmol, 1 eq) in THE (10 mL) was added dropwise DMP (2.85 g, 6.73 mmol, 2.08 mL, 2.5 eq) at 0° C. under N₂. The mixture was stirred at 15° C. for 2 h under N₂. The mixture was quenched with saturated aqueous NaHCO₃/Na₂S₂O₃=1:1 (120 mL). The reaction mixture was poured into water (45 mL) and extracted with DCM (50 mL×2). The combined organic layers were washed with brine (20 mL), dried over Na₂SO₄, filtered, and concentrated under reduced pressure to give crude 2,2,2-trifluoro-1-(4-(trifluoromethoxy)phenyl)ethan-1-one (0.6 g) as yellow oil. ¹H NMR (400 MHz, CDCl₃-d) δ 8.16 (d, 2H), 7.39 (d, 2H).

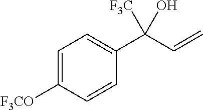 [SMILES: C=CC(O)(c1ccc(OC(F)(F)F)cc1)C(F)(F)F]

To a solution of 2,2,2-trifluoro-1-(4-(trifluoromethoxy)phenyl)ethan-1-one (0.45 g, 1.74 mmol, 1 eq) in THE (5 mL) was added bromo(vinyl)magnesium (1 M, 5.40 mL, 3.1 eq) at −20° C. under N₂. The mixture was stirred at −20° C. for 2 h. The reaction mixture was poured into saturated aqueous NH₄Cl (45 mL) and extracted with EtOAc (30 mL×2). The combined organic layers were washed with brine (20 mL), dried over Na₂SO₄, filtered, and concentrated under reduced pressure. The residue was purified by column chromatography (SiO₂, petroleum ether/EtOAc=70:30) to give 1,1,1-trifluoro-2-(4-(trifluoromethoxy)phenyl)but-3-en-2-ol (0.3 g, 1.05 mmol, 60.1% yield) as white oil. ¹H NMR (400 MHz, CDCl₃-d) δ 7.63 (d, 2H), 7.25 (d, 2H), 6.40 (s, 1H), 5.68-5.51 (m, 2H).

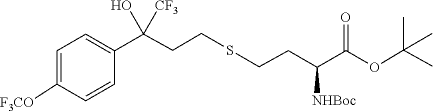 [SMILES: CC(C)(C)OC(=O)N[C@@H](CCSCCC(O)(c1ccc(OC(F)(F)F)cc1)C(F)(F)F)C(=O)OC(C)(C)C]

A mixture of 1,1,1-trifluoro-2-(4-(trifluoromethoxy)phenyl)but-3-en-2-ol (0.3 g, 1.05 mmol, 1 eq), tert-butyl (tert-butoxycarbonyl)-L-homocysteinate (366 mg, 1.26 mmol, 1.2 eq), AIBN (51.6 mg, 314 μmol, 0.3 eq) in MeOH (0.03 mL) and H₂O (0.01 mL) was degassed and purged 3 times with Ar, and then the mixture was stirred at 80° C. for 16 h. The reaction mixture was poured into water (25 mL) and extracted with EtOAc (10 mL×2). The combined organic layers were washed with brine (20 mL), dried over Na₂SO₄, filtered, and concentrated under reduced pressure to give a residue. The residue was purified by column chromatography (SiO₂, petroleum ether/EtOAc=3:1) to give tert-butyl N-(tert-butoxycarbonyl)-S-(4,4,4-trifluoro-3-hydroxy-3-(4-(trifluoromethoxy)phenyl)butyl)-L-homocysteinate (0.3 g, 519 μmol, 49.5% yield) as white oil. ¹H NMR (400 MHz, CDCl₃-d) δ 7.61 (br d, 2H), 7.25 (br s, 2H), 5.24-5.05 (m, 1H), 4.13 (q, 1H), 2.75-2.66 (m, 1H), 2.65-2.36 (m, 6H), 2.04-1.77 (m, 3H), 1.47-1.44 (m, 18H).

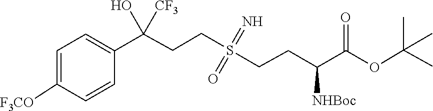 [SMILES: CC(C)(C)OC(=O)N[C@@H](CCS(=N)(=O)CCC(O)(c1ccc(OC(F)(F)F)cc1)C(F)(F)F)C(=O)OC(C)(C)C]

A mixture of tert-butyl N-(tert-butoxycarbonyl)-S-(4,4,4-trifluoro-3-hydroxy-3-(4-(trifluoromethoxy)phenyl)butyl)-L-homocysteinate (0.28 g, 484 μmol, 1 eq), ammonium carbamate (302 mg, 3.88 mmol, 8 eq) and PhI(OAc)₂ (624 mg, 1.94 mmol, 4 eq) in i-PrOH (4 mL) was stirred at 15° C. for 16 h. The reaction mixture was concentrated under reduced pressure and the resultant residue was diluted with water (25 mL) and extracted with DCM (10 mL×2). The combined organic layers were washed with brine (20 mL), dried over Na₂SO₄, filtered, and concentrated under reduced pressure. The residue was purified by column chromatography (SiO₂, petroleum ether/EtOAc=70:30) to give tert-butyl (2S)-2-((tert-butoxycarbonyl)amino)-4-(4,4,4-trifluoro-3-hydroxy-3-(4-(trifluoromethoxy)phenyl)butylsulfonimidoyl)butanoate (0.11 g, 180 μmol, 37.2% yield) as a white oil. ¹H NMR (400 MHz, CDCl₃-d) δ 7.65 (dd, 2H), 7.29-7.27 (m, 1H), 7.25 (br s, 1H), 5.31-5.11 (m, 1H), 4.32-4.17 (m, 1H), 3.33-3.01 (m, 3H), 2.99-2.87 (m, 1H), 2.82-2.59 (m, 2H), 2.45-2.25 (m, 1H), 2.18-2.06 (m, 1H), 1.53-1.44 (m, 18H).

A solution of tert-butyl (2S)-2-((tert-butoxycarbonyl)amino)-4-(4,4,4-trifluoro-3-hydroxy-3-(4-(trifluoromethoxy)phenyl)butylsulfonimidoyl)butanoate (0.11 g, 180 μmol, 1 eq) in HCl/dioxane (4 M, 8 mL) was stirred at 15° C. for 16 h. The reaction mixture was concentrated under reduced pressure. The residue was purified by prep-HPLC (column: Waters Xbridge BEH C18 (100×30 mm, 10 μm); mobile phase: [H₂O (10 mM NH₄HCO₃)-MeCN]; gradient: 10-40% B, 8.0 min) to give (2S)-2-amino-4-(4,4,4-trifluoro-3-hydroxy-3-(4-(trifluoromethoxy)phenyl)butylsulfonimidoyl)butanoic acid (45 mg, 97.4 μmol, 53.9% yield) as white solid. LCMS: Rt=2.346 min., (ES⁺) m/z (M+H)⁺=453.1, HPLC Conditions: B. ¹H NMR (400 MHz, MeOD-d₄) δ 7.74 (d, 2H), 7.35 (br d, 2H), 3.72-3.61 (m, 1H), 3.20-3.20 (m, 1H), 3.30-3.08 (m, 2H), 2.81-2.68 (m, 2H), 2.65-2.51 (m, 1H), 2.27 (q, 2H).

### Exemplary Embodiment 1aa20

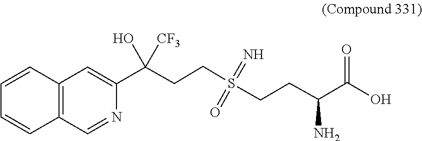 [SMILES: N=S(=O)(CC[C@H](N)C(=O)O)CCC(O)(c1cc2ccccc2cn1)C(F)(F)F]

### (2S)-2-amino-4-(4,4,4-trifluoro-3-hydroxy-3-(isoquinolin-3-yl)butylsulfonimidoyl)butanoic acid

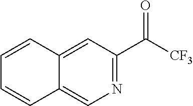 [SMILES: O=C(c1cc2ccccc2cn1)C(F)(F)F]

To a solution of methyl isoquinoline-3-carboxylate (2.44 g, 13.0 mmol, 1 eq) in THE (30 mL) was added TMSCF₃ (9.27 g, 65.2 mmol, 5 eq) and TBAF (1 M, 1.30 mL, 0.1 eq) at −15° C. The mixture was stirred at 20° C. for 2 h. The reaction mixture was diluted with H₂O (50 mL) and extracted with EtOAc (50 mL×3). The combined organic phase was washed with brine (30 mL), dried with anhydrous Na₂SO₄, filtered, and concentrated under reduced pressure. The residue was purified by prep-TLC (SiO₂, petroleum ether/EtOAc=3:1) to give 2,2,2-trifluoro-1-(isoquinolin-3-yl)ethan-1-one (1.97 g, 8.75 mmol, 67.1% yield) as a yellow oil. ¹H NMR (400 MHz, CDCl₃-d) δ 9.39 (s, 1H), 8.67 (s, 1H), 8.18-7.98 (m, 3H), 7.87-7.85 (m 1H).

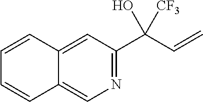 [SMILES: C=CC(O)(c1cc2ccccc2cn1)C(F)(F)F]

To a solution of 2,2,2-trifluoro-1-(isoquinolin-3-yl)ethan-1-one (1.67 g, 7.42 mmol, 1 eq) in THE (30 mL) was added bromo(vinyl)magnesium (1 M, 22.3 mL, 3 eq) at −65° C. The mixture was stirred at −65° C. for 1 h before quenching with saturated aqueous NH₄Cl (50 mL). The mixture was extracted with EtOAc (50 mL×3) and the combined organic phase was washed with brine (10 mL), dried with anhydrous Na₂SO₄, filtered, and concentrated under reduced pressure. The residue was purified by prep-TLC (SiO₂, petroleum ether/EtOAc=5:1) to give 1,1,1-trifluoro-2-(isoquinolin-3-yl)but-3-en-2-ol (350 mg, 1.38 mmol, 18.6% yield) as a yellow solid. ¹H NMR (400 MHz, CDCl₃-d) δ 9.24 (s, 1H), 8.05 (d, 1H), 7.96-7.89 (m, 2H), 7.85-7.62 (m, 2H), 6.47 (dd, 1H), 5.87 (d, 1H), 5.50 (d, 1H).

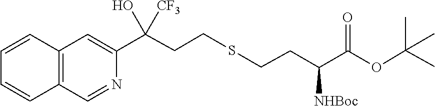 [SMILES: CC(C)(C)OC(=O)N[C@@H](CCSCCC(O)(c1cc2ccccc2cn1)C(F)(F)F)C(=O)OC(C)(C)C]

A mixture of 1,1,1-trifluoro-2-(isoquinolin-3-yl)but-3-en-2-ol (300 mg, 1.18 mmol, 1 eq), tert-butyl (tert-butoxycarbonyl)-L-homocysteinate (518 mg, 1.78 mmol, 1.5 eq), and AIBN (58.4 mg, 355 μmol, 0.3 eq) in MeOH (1 mL)/H₂O (0.1 mL) was degassed and purged 3 times with N₂, and then the mixture was stirred at 80° C. for 12 hr under N₂ atmosphere. The reaction mixture was diluted with H₂O (20 mL) and extracted with EtOAc (20 mL×3). The combined organic phase was washed with brine (10 mL), dried with anhydrous Na₂SO₄, filtered, and concentrated under reduced pressure. The residue was purified by prep-TLC (SiO₂, petroleum ether/EtOAc=5:1) to give tert-butyl N-(tert-butoxycarbonyl)-S-(4,4,4-trifluoro-3-hydroxy-3-(isoquinolin-3-yl)butyl)-L-homocysteinate (471 mg, 865 μmol, 73.0% yield) as a yellow oil. ¹H NMR (400 MHz, CDCl₃-d) δ 9.25 (s, 1H), 8.07 (d, 1H), 7.98-7.87 (m, 2H), 7.81 (t, 1H), 7.75-7.67 (m, 1H), 5.08 (br d, 1H), 4.34-4.21 (m, 1H), 2.71-2.43 (m, 6H), 2.02-1.96 (m, 1H), 1.88-1.76 (m, 1H), 1.43 (d, 18H).

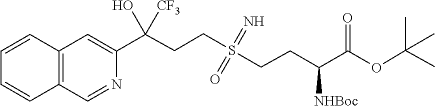 [SMILES: CC(C)(C)OC(=O)N[C@@H](CCS(=N)(=O)CCC(O)(c1cc2ccccc2cn1)C(F)(F)F)C(=O)OC(C)(C)C]

To a solution of tert-butyl N-(tert-butoxycarbonyl)-S-(4,4,4-trifluoro-3-hydroxy-3-(isoquinolin-3-yl)butyl)-L-homocysteinate (160 mg, 294 μmol, 1 eq) in i-PrOH (5 mL) was added PhI(OAc)₂ (379 mg, 1.18 mmol, 4 eq) and ammonium carbamate (184 mg, 2.35 mmol, 8 eq). The mixture was stirred at 20° C. for 12 hr. The reaction mixture was concentrated under reduced pressure and the residue was diluted with H₂O (10 mL) and extracted with DCM (10 mL×3). The combined organic phase was washed with brine (5 mL), dried with anhydrous Na₂SO₄, filtered, and concentrated under reduced pressure. The residue was purified by prep-TLC (SiO₂, petroleum ether/EtOAc=2:1) to give tert-butyl (2S)-2-((tert-butoxycarbonyl)amino)-4-(4,4,4-trifluoro-3-hydroxy-3-(isoquinolin-3-yl)butylsulfonimidoyl)butanoate (90 mg, 156 μmol, 53.2% yield) as a yellow oil. ¹H NMR (400 MHz, CDCl₃-d) δ 9.27 (s, 1H), 8.10-7.94 (m, 3H), 7.83 (dt, 1H), 7.75 (d, 1H), 5.28-5.17 (m, 1H), 4.27 (br dd, 1H), 3.29-2.99 (m, 3H), 2.88-2.66 (m, 3H), 2.43-2.31 (m, 1H), 2.10-2.01 (m, 1H), 1.49-1.44 (m, 18H).

A solution of tert-butyl (2S)-2-((tert-butoxycarbonyl)amino)-4-(4,4,4-trifluoro-3-hydroxy-3-(isoquinolin-3-yl)butylsulfonimidoyl)butanoate (90 mg, 156 μmol, 1 eq) in HCl/dioxane (6 M, 5 mL) was stirred at 25° C. for 7 hr. The reaction mixture was concentrated under reduced pressure and the residue was purified by prep-HPLC (column: Waters Xbridge Prep OBD C18 (150×40 mm, 10 μm); mobile phase: [NH₄HCO₃-MeCN]; gradient: 15%-45% B over 8.0 min=) to give (2S)-2-amino-4-(4,4,4-trifluoro-3-hydroxy-3-(isoquinolin-3-yl)butylsulfonimidoyl)butanoic acid (31.8 mg, 74.1 μmol, 47.4% yield) as a white solid. LCMS: Rt=2.068 min, (ES⁺) m/z (M+H)⁺=420.0, HPLC Conditions: B. ¹H NMR (400 MHz, D₂O) δ 9.27 (s, 1H), 8.22 (s, 1H), 8.16 (br d, 1H), 8.02 (br d, 1H), 7.87 (t, 1H), 7.78 (br d, 1H), 3.85-3.74 (m, 1H), 3.46-3.21 (m, 3H), 3.05-2.60 (m, 3H), 2.29-2.17 (m, 2H).

### Exemplary Embodiment 1aa21

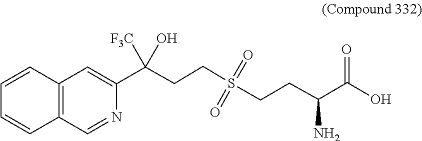 [SMILES: N[C@@H](CCS(=O)(=O)CCC(O)(c1cc2ccccc2cn1)C(F)(F)F)C(=O)O]

### (2S)-2-amino-4-((4,4,4-trifluoro-3-hydroxy-3-(isoquinolin-3-yl)butyl)sulfonyl)butanoic acid

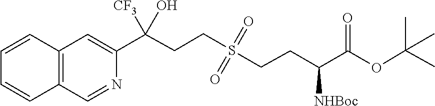 [SMILES: CC(C)(C)OC(=O)N[C@@H](CCS(=O)(=O)CCC(O)(c1cc2ccccc2cn1)C(F)(F)F)C(=O)OC(C)(C)C]

To a solution of tert-butyl (2S)-2-(tert-butoxycarbonylamino)-4-[4,4,4-trifluoro-3-hydroxy-3-(3-isoquinolyl)butyl]sulfanyl-butanoate (100 mg, 184 μmol, 1 eq) in DCM (2 mL) was added m-CPBA (93.2 mg, 459 μmol, 85% purity, 2.5 eq) at 0° C. The mixture was stirred at 20° C. for 12 hr. Solids were removed by filtration and the filtrate was washed with saturated aqueous Na₂S₂O₃ (10 mL) and NaHCO₃ (10 mL). The organic layer was dried over Na₂SO₄, filtered, and concentrated under reduced pressure. The residue was purified by prep-TLC (SiO₂, petroleum ether:EtOAc=1:1) to give tert-butyl (2S)-2-((tert-butoxycarbonyl)amino)-4-((4,4,4-trifluoro-3-hydroxy-3-(isoquinolin-3-yl)butyl)sulfonyl)butanoate (50 mg, 86.7 μmol, 47.2% yield) as a yellow oil. ¹H NMR (400 MHz, CDCl₃-d) δ 9.19 (s, 1H), 8.01-7.99 (m, 1H), 7.91-7.89 (m, 1H), 7.76 (t, 1H), 7.70-7.64 (m, 1H), 7.54-7.48 (m, 1H), 7.39-7.32 (m, 1H), 5.08 (br dd, 1H), 4.25-4.10 (m, 1H), 3.19-3.05 (m, 1H), 3.13-2.79 (m, 2H), 2.73-2.41 (m, 3H), 2.27 (br d, 1H), 2.04-1.91 (m, 1H), 1.40-1.34 (m, 18H).

A solution of tert-butyl (2S)-2-((tert-butoxycarbonyl)amino)-4-((4,4,4-trifluoro-3-hydroxy-3-(isoquinolin-3-yl)butyl)sulfonyl)butanoate (50 mg, 86.7 μmol, 1 eq) in HCl/dioxane (4M) (5 mL) was stirred at 20° C. for 12 hr. The reaction mixture was concentrated under reduced pressure and the residue was purified by prep-HPLC (column: Phenomenex C18 (75×30 mm, 3 μm); mobile phase: [water (NH₄HCO₃)-MeCN], gradient: 1-10% B, 10 min) to give (2S)-2-amino-4-((4,4,4-trifluoro-3-hydroxy-3-(isoquinolin-3-yl)butyl)sulfonyl)butanoic acid (15.0 mg, 35.6 μmol, 41.09% yield) as a white solid. LCMS: Rt=2.212 min., (ES⁺) m/z (M+H)⁺=421.0, HPLC Conditions: B. ¹H NMR (400 MHz, D₂O) δ=9.22 (s, 1H), 8.23-8.08 (m, 2H), 7.98 (d, 1H), 7.84-7.67 (m, 2H), 3.74 (t, 1H), 3.34-3.17 (m, 3H), 2.99-2.90 (m, 1H), 2.85-2.75 (m, 1H), 2.63 (br dd, 1H), 2.19 (br d, 2H).

### Exemplary Embodiment 1aa21.2

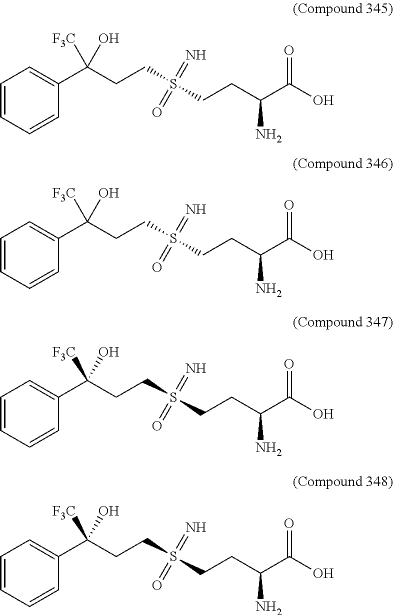 [SMILES: N=[S@](=O)(CC[C@H](N)C(=O)O)CCC(O)(c1ccccc1)C(F)(F)F ; N=[S@](=O)(CC[C@H](N)C(=O)O)CCC(O)(c1ccccc1)C(F)(F)F ; N=[S@@](=O)(CC[C@H](N)C(=O)O)CC[C@@](O)(c1ccccc1)C(F)(F)F ; N=[S@@](=O)(CC[C@H](N)C(=O)O)CC[C@@](O)(c1ccccc1)C(F)(F)F]

Compound 345 (Isomer 1), Compound 346 (Isomer 2), Compound 347 (Isomer 3), & Compound 348 (Isomer 4). Compounds 345 and 346 are diastereomers of each other, differing in the stereochemistry at the tetrahedral carbon bearing a hydroxyl group and a trifluoromethyl and a phenyl group.

### (2S)-2-amino-4-((S)-4,4,4-trifluoro-3-hydroxy-3-phenylbutylsulfonimidoyl)butanoic acid, (2S)-2-amino-4-((S)-4,4,4-trifluoro-3-hydroxy-3-phenylbutylsulfonimidoyl)butanoic acid, (S)-2-amino-4-((R,3R)-4,4,4-trifluoro-3-hydroxy-3-phenylbutylsulfonimidoyl)butanoic acid, & (S)-2-amino-4-((R,3S)-4,4,4-trifluoro-3-hydroxy-3-phenylbutylsulfonimidoyl)butanoic acid

### Synthesis Stage 1

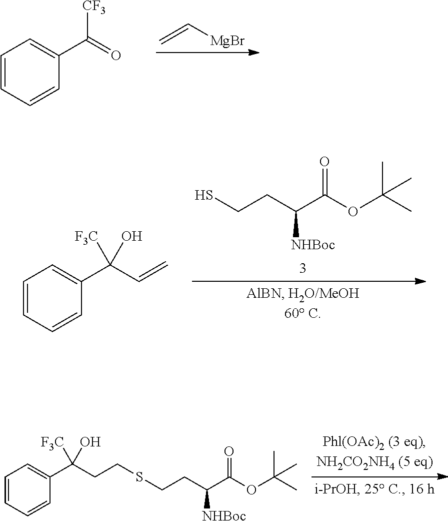

### Synthesis Stage 2

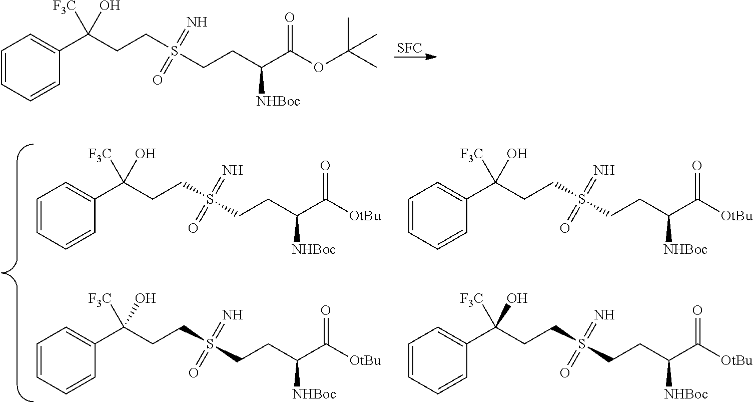

### Synthesis Stage 3

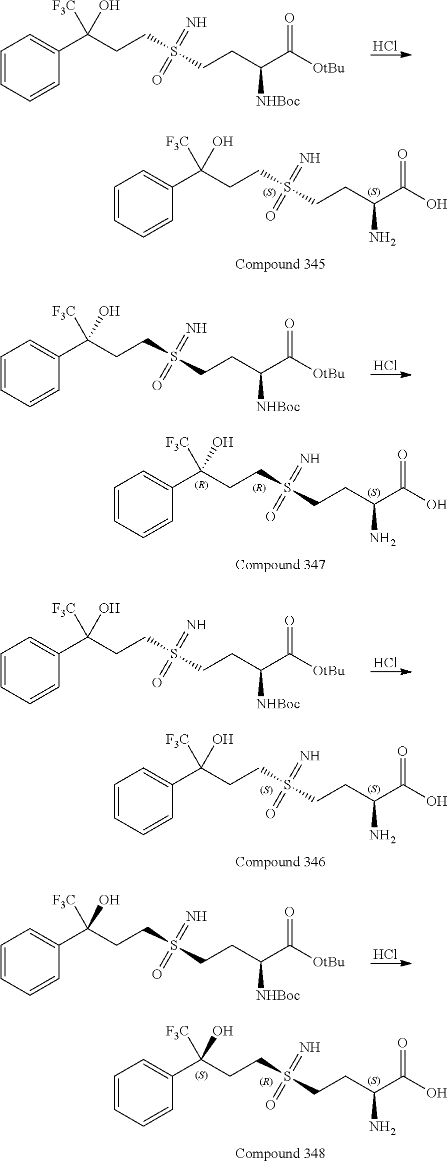

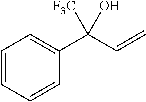

To a solution of 2,2,2-trifluoro-1-phenyl-ethanone (2.00 g, 11.4 mmol, 1.56 mL, 1 eq) in THE (20 mL) was added bromo(vinyl)magnesium (1 M, 11.72 mL, 1.02 eq) slowly at −65° C. The mixture was stirred at 20° C. for 12 h. The reaction mixture was diluted with H₂O (20 mL) and extracted with EtOAc (25 mL×3). The combined organic phase was washed with brine (20 mL), dried with anhydrous Na₂SO₄, filtered, and concentrated under reduced pressure to give a residue. The residue was purified by column chromatography (SiO₂, petroleum ether/EtOAc=1:0 to 5:1) to give 1,1,1-trifluoro-2-phenylbut-3-en-2-ol (1.94 g, 8.64 mmol, 75.1% yield), as yellow oil. ¹H NMR (400 MHz, CDCl₃-d) δ 7.60 (br d, 2H), 7.47-7.35 (m, 3H), 6.45 (dd, 1H), 5.63 (d, 1H), 5.53 (d, 1H), 2.57 (s, 1H).

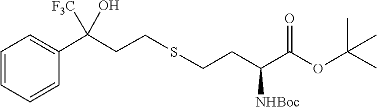 [SMILES: CC(C)(C)OC(=O)N[C@@H](CCSCCC(O)(c1ccccc1)C(F)(F)F)C(=O)OC(C)(C)C]

To a solution of 1,1,1-trifluoro-2-phenylbut-3-en-2-ol (500 mg, 2.47 mmol, 1.00 eq) in H₂O (8.50 mL) and MeOH (8.50 mL) was added tert-butyl (tert-butoxycarbonyl)-L-homocysteinate (792 mg, 2.72 mmol, 1.10 eq) and AIBN (40.6 mg, 247 μmol, 0.100 eq), then the mixture was stirred at 60° C. for 24 h. The mixture was concentrated under reduced pressure and the residue was diluted with H₂O (20 mL) and extracted with DCM (15 mL×3). The combined organic phase was washed with brine (30 mL), dried with anhydrous Na₂SO₄, filtered, and concentrated under reduced pressure. The residue was purified by column chromatography (SiO₂, Petroleum ether/Ethyl acetate=10:1 to 1:1) to obtain (2S)-tert-butyl 2-((tert-butoxycarbonyl)amino)-4-((4,4,4-trifluoro-3-hydroxy-3-phenylbutyl)thio)butanoate (620 mg, 1.13 mmol, 45.7% yield) as colorless oil. ¹H NMR (400 MHz, CDCl₃-d) δ 7.64-7.51 (m, 2H), 7.49-7.34 (m, 3H), 5.27-5.02 (m, 1H), 4.39-4.19 (m, 1H), 2.63-2.43 (m, 4H), 2.41-2.25 (m, 2H), 2.14-1.78 (m, 2H), 1.50-1.43 (m, 18H).

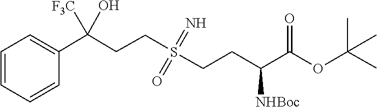 [SMILES: CC(C)(C)OC(=O)N[C@@H](CCS(=N)(=O)CCC(O)(c1ccccc1)C(F)(F)F)C(=O)OC(C)(C)C]

To a solution of (2S)-tert-butyl 2-((tert-butoxycarbonyl)amino)-4-((4,4,4-trifluoro-3-hydroxy-3-phenylbutyl)thio)butanoate (400 mg, 810 μmol, 1.00 eq) in i-PrOH (4.00 mL) was added PhI(OAc)₂ (1.04 g, 3.24 mmol, 4.00 eq) and ammonium carbamate (506 mg, 6.48 mmol, 8.00 eq). The mixture was stirred at 25° C. for 16 h. The reaction mixture was concentrated under reduced pressure to remove solvent. The residue was purified by prep-HPLC (column: C18-1 (150×30 mm, 5 μm); mobile phase: [water (FA)-MeCN]; gradient: 30-60% B, 10 min) to obtain (2S)-tert-butyl 2-((tert-butoxycarbonyl)amino)-4-(4,4,4-trifluoro-3-hydroxy-3-phenylbutylsulfonimidoyl)butanoate (300 mg, 543 μmol, 67.0% yield) as white solid. ¹H NMR (400 MHz, CDCl₃-d) δ 7.67-7.53 (m, 2H), 7.51-7.35 (m, 3H), 5.36-5.10 (m, 1H), 4.38-4.19 (m, 1H), 3.34-2.28 (m, 7H), 2.18-2.07 (m, 1H), 1.50-1.43 (m, 18H).

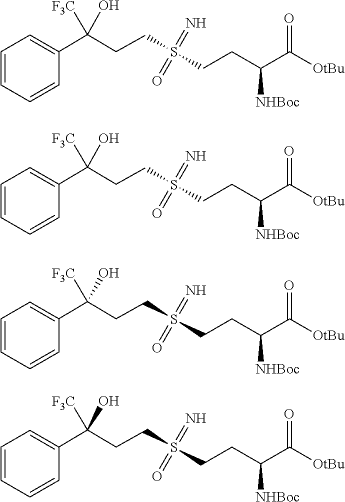 [SMILES: CC(C)(C)OC(=O)N[C@@H](CC[S@](=N)(=O)CCC(O)(c1ccccc1)C(F)(F)F)C(=O)OC(C)(C)C ; CC(C)(C)OC(=O)N[C@@H](CC[S@](=N)(=O)CCC(O)(c1ccccc1)C(F)(F)F)C(=O)OC(C)(C)C ; CC(C)(C)OC(=O)N[C@@H](CC[S@@](=N)(=O)CC[C@@](O)(c1ccccc1)C(F)(F)F)C(=O)OC(C)(C)C ; CC(C)(C)OC(=O)N[C@@H](CC[S@@](=N)(=O)CC[C@](O)(c1ccccc1)C(F)(F)F)C(=O)OC(C)(C)C]

(2S)-tert-butyl 2-((tert-butoxycarbonyl)amino)-4-(4,4,4-trifluoro-3-hydroxy-3-phenylbutylsulfonimidoyl)butanoate was separated by prep-HPLC (column: Phenomenex-Cellulose-2 (250×30 mm, 10 μm); mobile phase: [Heptane-EtOH]; gradient: 10% B then column: Phenomenex-Cellulose-2 (250×30 mm, 5 μm); mobile phase: [Hexane-IPA]; gradient: 15-25% B, 10 min). The separated compounds were tentatively assigned stereochemistry and notated by their peak elution order. tert-butyl (2S)-2-((tert-butoxycarbonyl)amino)-4-((S)-4,4,4-trifluoro-3-hydroxy-3-phenylbutylsulfonimidoyl)butanoate (Peak 1, 40.0 mg, 75.4 μmol, 15.8% yield) was obtained as white solid. tert-butyl (2S)-2-((tert-butoxycarbonyl)amino)-4-((S)-4,4,4-trifluoro-3-hydroxy-3-phenylbutylsulfonimidoyl)butanoate (Peak 2, 10 mg, 19.06 μmol, 10.00% yield) was obtained as white solid. tert-butyl (S)-2-((tert-butoxycarbonyl)amino)-4-((R,3R)-4,4,4-trifluoro-3-hydroxy-3-phenylbutylsulfonimidoyl)butanoate (Peak 3, 50.0 mg, 95.3 μmol, 50.0% yield) was obtained as white solid. tert-butyl (S)-2-((tert-butoxycarbonyl)amino)-4-((R,3S)-4,4,4-trifluoro-3-hydroxy-3-phenylbutylsulfonimidoyl)butanoate (Peak 4, 40.0 mg, 74.7 μmol, 15.6% yield) was obtained as white solid.

A solution of tert-butyl (2S)-2-((tert-butoxycarbonyl)amino)-4-((S)-4,4,4-trifluoro-3-hydroxy-3-phenylbutylsulfonimidoyl)butanoate (Peak 1, 40.0 mg, 76.2 μmol, 1.00 eq) in HCl/dioxane (4.00 M, 5.00 mL, 262 eq) was stirred at 15° C. for 12 h. The reaction mixture was concentrated under reduced pressure. The residue was purified by prep-HPLC (column: C18-1 (150×30 mm, 5 μm); mobile phase: [water (FA)-MeCN]; gradient: 15-45% B, 10 min) to give (2S)-2-amino-4-((S)-4,4,4-trifluoro-3-hydroxy-3-phenylbutylsulfonimidoyl)butanoic acid (Isomer 1, 11.7 mg, 28.2 μmol, 37.0% yield, FA) as a white solid. LCMS: Rt=1.770 min, (ES⁺) m/z (M+H)⁺=369.0, HPLC Conditions: C. ¹H NMR (400 MHz, D₂O) δ 7.63 (br d, 2H), 7.58-7.45 (m, 3H), 3.92-3.74 (m, 1H), 3.51-3.18 (m, 3H), 3.00-2.52 (m, 3H), 2.36-2.14 (m, 2H).

A solution of tert-butyl (2S)-2-((tert-butoxycarbonyl)amino)-4-((S)-4,4,4-trifluoro-3-hydroxy-3-phenylbutylsulfonimidoyl)butanoate (Peak 2, 10.0 mg, 19.0 μmol, 1.00 eq) in HCl/dioxane (4.00 M, 2.00 mL, 210 eq) was stirred at 15° C. for 12 h. The reaction mixture was concentrated under reduced pressure. The residue was purified by prep-HPLC (column: C18-1 (150×30 mm, 5 μm); mobile phase: [water (FA)-MeCN]; gradient: 15-45% B, 10 min) to give (2S)-2-amino-4-((S)-4,4,4-trifluoro-3-hydroxy-3-phenylbutylsulfonimidoyl)butanoic acid (Isomer 2, 2.10 mg, 5.07 μmol, 5.33% yield, FA) as a white solid. LCMS: Rt=1.774 min, (ES⁺) m/z (M+H)⁺=369.1, HPLC Conditions: C. ¹H NMR (400 MHz, D₂O) δ 7.61 (br d, 2H), 7.5 (br d, 3H), 3.90-3.70 (m, 1H), 3.45-3.20 (m, 3H), 3.02-2.50 (m, 3H), 2.39-2.10 (m, 2H).

A solution of tert-butyl (S)-2-((tert-butoxycarbonyl)amino)-4-((R,3R)-4,4,4-trifluoro-3-hydroxy-3-phenylbutylsulfonimidoyl)butanoate (Peak 3, 49.8 mg, 95.0 μmol, 1.00 eq) in HCl/dioxane (4.00 M, 5.00 mL, 210 eq) was stirred at 15° C. for 12 h. The reaction mixture was concentrated under reduced pressure. The residue was purified by prep-HPLC (column: C18-1 (150×30 mm, 5 μm); mobile phase: [water (FA)-MeCN]; gradient: 15-45% B, 10 min) to give (S)-2-amino-4-((R,3R)-4,4,4-trifluoro-3-hydroxy-3-phenylbutylsulfonimidoyl)butanoic acid (Isomer 3, 10.6 mg, 25.7 μmol, 27.1% yield, FA) as a white solid. LCMS: Rt=1.767 min, (ES⁺) m/z (M+H)⁺=369.1, HPLC Conditions: C. ¹H NMR (400 MHz, D₂O) δ 7.66-7.58 (m, 2H), 7.55-7.44 (m, 3H), 3.88-3.74 (m, 1H), 3.49-3.18 (m, 3H), 3.00-2.50 (m, 3H), 2.33-2.16 (m, 2H).

A solution of tert-butyl (S)-2-((tert-butoxycarbonyl)amino)-4-((R,3S)-4,4,4-trifluoro-3-hydroxy-3-phenylbutylsulfonimidoyl)butanoate (Peak 4, 40.0 mg, 76.2 μmol, 1.00 eq) in HCl/dioxane (4.00 M, 4.00 mL, 210 eq) was stirred at 15° C. for 12 h. The reaction mixture was concentrated under reduced pressure. The residue was purified by prep-HPLC (column: C18-1 (150×30 mm, 5 μm); mobile phase: [water (FA)-MeCN]; gradient: 15-45% B, 10 min) to give (S)-2-amino-4-((R,3S)-4,4,4-trifluoro-3-hydroxy-3-phenylbutylsulfonimidoyl)butanoic acid (Isomer 4, 7.63 mg, 18.4 μmol, 24.1% yield, FA) as a white solid. LCMS: Rt=1.772 min, (ES⁺) m/z (M+H)⁺=369.1, HPLC Conditions: C. ¹H NMR (400 MHz, D₂O) δ 7.62 (br d, 2H), 7.56-7.46 (m, 3H), 3.93-3.73 (m, 1H), 3.48-3.18 (m, 3H), 3.00-2.54 (m, 3H), 2.32-2.19 (m, 2H).

The compounds described in Table 13 were prepared using the general methods outlined above.

TABLE 13
Characterization of Compounds 333-348

| Compound | | MS | HPLC | HPLC |
|---|---|---|---|---|
| # | Structure | (M + H)⁺ | Rt | cond. |
| 333 | 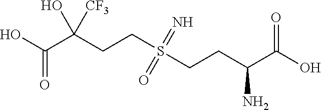 [SMILES: N=S(=O)(CC[C@H](N)C(=O)O)CCC(O)(C(=O)O)C(F)(F)F] | 337.0 | 0.763 | G |
| | ¹H NMR (400 MHz, D₂O) δ 3.86-3.72 (m, 1H), | | | |
| | 3.60-2.88 (m, 4H), 2.65-2.13 (m, 4H) | | | |
| 334 | 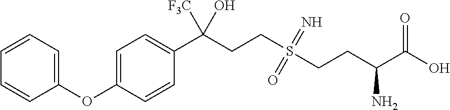 [SMILES: N=S(=O)(CC[C@H](N)C(=O)O)CCC(O)(c1ccc(Oc2ccccc2)cc1)C(F)(F)F] | 461.1 | 2.04 | C |
| | ¹H NMR (400 MHz, MeOD-d₄) δ 7.58 (d, 2H), | | | |
| | 7.41-7.35 (m, 2H), 7.20-7.10 (m, 1H), 7.02 (t, | | | |
| | 4H), 3.73-3.63 (m, 1H), 3.51-3.34 (m, 1H), | | | |
| | 3.28-3.11 (m, 2H), 2.84-2.67 (m, 2H), 2.62- | | | |
| | 2.48 (m, 1H), 2.28 (q, 2H). | | | |
| 335 | 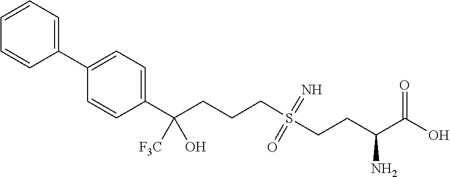 [SMILES: N=S(=O)(CCCC(O)(c1ccc(-c2ccccc2)cc1)C(F)(F)F)CC[C@H](N)C(=O)O] | 459.2 | 2.06 | C |
| | ¹H NMR (400 MHz, MeOD-d₄) δ 7.72-7.61 (m, | | | |
| | 6H), 7.48-7.41 (m, 2H), 7.39-7.32 (m, 1H), | | | |
| | 3.73-3.61 (m, 1H), 3.29-3.07 (m, 4H), 2.53- | | | |
| | 2.40 (m, 1H), 2.35-2.13 (m, 3H), 1.99-1.82 (m, | | | |
| | 1H), 1.68-1.52 (m, 1H) | | | |
| 336 | 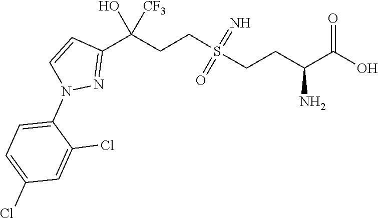 [SMILES: N=S(=O)(CC[C@H](N)C(=O)O)CCC(O)(c1ccn(-c2ccc(Cl)cc2Cl)n1)C(F)(F)F] | 503.1 | 1.96 | C |
| | ¹H NMR (400 MHz, MeOD-d₄) δ 8.03 (br s, 1H), | | | |
| | 7.71 (s, 1H), 7.65-7.58 (m, 1H), 7.55-7.48 (m, | | | |
| | 1H), 6.70 (s, 1H), 3.75-3.64 (m, 1H), 3.41- | | | |
| | 3.32 (m, 2H), 3.30-3.21 (m, 1H), 3.18-3.03 (m, | | | |
| | 1H), 2.86-2.72 (m, 1H), 2.59-2.45 (m, 1H), | | | |
| | 2.41-2.23 (m, 2H) | | | |
| 337 | 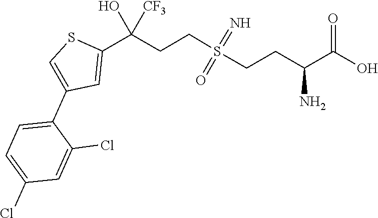 [SMILES: N=S(=O)(CC[C@H](N)C(=O)O)CCC(O)(c1cc(-c2ccc(Cl)cc2Cl)cs1)C(F)(F)F] | 519.0 | 2.20 | C |
| | ¹H NMR (400 MHz, DMSO-d₆) δ 7.64 (s, 1H), | | | |
| | 7.55 (s, 1H), 7.55-7.49 (m, 1H), 7.47-7.35 (m, | | | |
| | 2H), 3.70-3.65(m, 1H), 3.45-3.30 (m, 3H), | | | |
| | 3.10-2.80 (m, 1H), 2.67-2.63 (m, 2H), 2.33- | | | |
| | 2.28 (m, 2H) | | | |
| 338 | 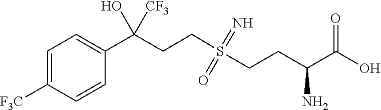 [SMILES: N=S(=O)(CC[C@H](N)C(=O)O)CCC(O)(c1ccc(C(F)(F)F)cc1)C(F)(F)F] | 437.1 | 2.32 | B |
| | ¹H NMR (400 MHz, MeOD-d₄) δ 7.89-7.81 (m, | | | |
| | 2H), 7.79-7.70 (m, 2H), 3.67-3.54 (m, 1H), | | | |
| | 3.42-3.34 (m, 1H), 3.30-3.12 (m, 2H), 2.85- | | | |
| | 2.54 (m, 3H), 2.38-2.13 (m, 2H) | | | |
| 339 | 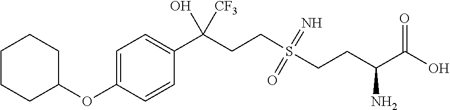 [SMILES: N=S(=O)(CC[C@H](N)C(=O)O)CCC(O)(c1ccc(OC2CCCCC2)cc1)C(F)(F)F] | 467.2 | 2.15 | C |
| | ¹H NMR (400 MHz, MeOD-d₄) δ 7.48 (d, 2H), | | | |
| | 6.94 (d, 2H), 4.30 (s, 1H), 3.71-3.63 (m, 1H), | | | |
| | 3.40-3.33 (m, 1H), 3.29-3.11 (m, 2H), 2.83- | | | |
| | 2.63 (m, 2H), 2.59-2.45 (m, 1H), 2.33-2.21 (m, | | | |
| | 2H), 2.03-1.94 (m, 2H), 1.84-1.76 (m, 2H), | | | |
| | 1.64-1.33 (m, 6H) | | | |
| 340 | 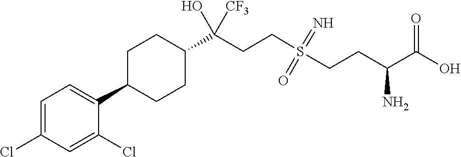 [SMILES: N=S(=O)(CC[C@H](N)C(=O)O)CCC(O)([C@H]1CC[C@H](c2ccc(Cl)cc2Cl)CC1)C(F)(F)F] | 519.1 | 2.404 | C |
| | ¹H NMR (400 MHz, MeOD-d₄) δ 7.41 (d, 1H), | | | |
| | 7.35-7.26 (m, 2H), 3.75-3.66 (m, 1H), 3.51- | | | |
| | 3.33 (m, 4H), 3.08-2.96 (m, 1H), 2.39-2.21 (m, | | | |
| | 4H), 2.07 (br d, 2H), 2.01-1.85 (m, 3H), 1.53- | | | |
| | 1.40 (m, 4H) | | | |
| 341 | 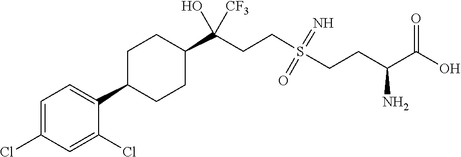 [SMILES: N=S(=O)(CC[C@H](N)C(=O)O)CCC(O)([C@H]1CC[C@@H](c2ccc(Cl)cc2Cl)CC1)C(F)(F)F] | 519.1 | 2.383 | C |
| | ¹H NMR (400 MHz, MeOD-d₄) δ 7.56 (d, 1H), | | | |
| | 7.42 (d, 1H), 7.31 (dd, 1H), 3.73-3.67 (m, 1H), | | | |
| | 3.50-3.34 (m, 3H), 3.28-3.21 (m, 1H), 2.40- | | | |
| | 2.30 (m, 2H), 2.24 (br dd, 2H), 2.16-2.08 (m, | | | |
| | 2H), 2.01-1.73 (m, 6H), 1.67-1.54 (m, 2H) | | | |
| 342 | 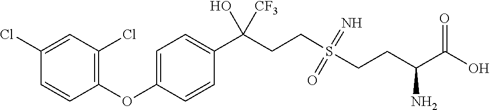 [SMILES: N=S(=O)(CC[C@H](N)C(=O)O)CCC(O)(c1ccc(Oc2ccc(Cl)cc2Cl)cc1)C(F)(F)F] | 529.1 | 2.28 | C |
| | ¹H NMR (400 MHz, MeOD-d₄) δ 7.64-7.57 (m, | | | |
| | 3H), 7.35 (dd, 1H), 7.09 (dd, 1H), 6.99 (dd, 2H), | | | |
| | 3.72-3.63 (m, 1H), 3.41-3.35 (m, 1H), 3.23- | | | |
| | 3.16 (m, 2H), 2.83-2.66 (m, 2H), 2.62-2.50 (m, | | | |
| | 1H), 2.33-2.22 (m, 2H) | | | |
| 343 | 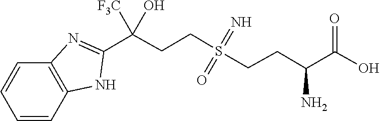 [SMILES: N=S(=O)(CC[C@H](N)C(=O)O)CCC(O)(c1nc2ccccc2[nH]1)C(F)(F)F] | 409.1 | 1.50 | B |
| | ¹H NMR (400 MHz, MeOD-d₄) δ 7.62 (dd, 5.7 Hz, | | | |
| | 2H), 7.29 (dd, 2H), 3.74-3.69 (m, 1H), 3.44- | | | |
| | 3.37 (m, 2H), 3.28-3.23 (m, 1H), 3.08-2.85 | | | |
| | (m, 2H), 2.72-2.57 (m, 1H), 2.39-2.22 (m, 2H) | | | |
| 344 | 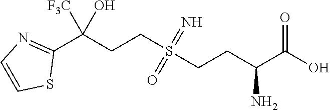 [SMILES: N=S(=O)(CC[C@H](N)C(=O)O)CCC(O)(c1nccs1)C(F)(F)F] | 376.1 | 1.71 | D |
| | ¹H NMR (400 MHz, D₂O) δ 7.89 (d, 1H), 7.72 | | | |
| | (d, 1H), 3.87-3.78 (m, 1H), 3.44-3.30 (m, 3H), | | | |
| | 3.03-2.92 (m, 1H), 2.83 (dt, 1H), 2.72-2.62 (m, | | | |
| | 1H), 2.34-2.26 (m, 2H) | | | |
| 345 | 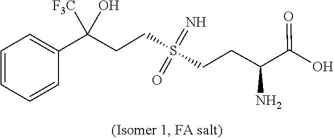 [SMILES: N=[S@](=O)(CC[C@H](N)C(=O)O)CCC(O)(c1ccccc1)C(F)(F)F] | 369.0 | 1.77 | C |
| | ¹H NMR (400 MHz, D₂O) δ 7.63 (br d, 2H), 7.58- | | | |
| | 7.45 (m, 3H), 3.92-3.74 (m, 1H), 3.51-3.18 | | | |
| | (m, 3H), 3.00-2.52 (m, 3H), 2.36-2.14 (m, 2H) | | | |
| 346 | 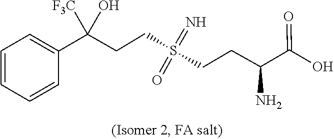 [SMILES: N=[S@](=O)(CC[C@H](N)C(=O)O)CCC(O)(c1ccccc1)C(F)(F)F] | 369.1 | 1.77 | C |
| | ¹H NMR (400 MHz, D₂O) δ 7.61 (br d, 2H), 7.50 | | | |
| | (br d, 3H), 3.90-3.70 (m, 1H), 3.45-3.20 (m, | | | |
| | 3H), 3.02-2.50 (m, 3H), 2.39-2.10 (m, 2H) | | | |
| 347 | 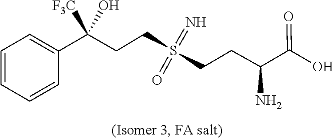 [SMILES: N=[S@@](=O)(CC[C@H](N)C(=O)O)CC[C@@](O)(c1ccccc1)C(F)(F)F] | 369.1 | 1.77 | C |
| | ¹H NMR (400 MHz, D₂O) δ 7.66-7.58 (m, 2H), | | | |
| | 7.55-7.44 (m, 3H), 3.88-3.74 (m, 1H), 3.49- | | | |
| | 3.18 (m, 3H), 3.00-2.50 (m, 3H), 2.33-2.16 (m, | | | |
| | 2H) | | | |
| 348 | 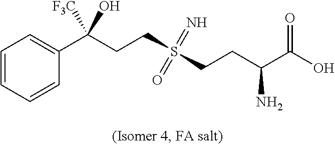 [SMILES: N=[S@@](=O)(CC[C@H](N)C(=O)O)CC[C@](O)(c1ccccc1)C(F)(F)F] | 369.1 | 1.77 | C |
| | ¹H NMR (400 MHz, D₂O) δ 7.62 (br d, 2H), 7.56- | | | |
| | 7.46 (m, 3H), 3.93-3.73 (m, 1H), 3.48-3.18 | | | |
| | (m, 3H), 3.00-2.54 (m, 3H), 2.32-2.19 (m, 2H) | | | |

### Exemplary Embodiment 1aa22

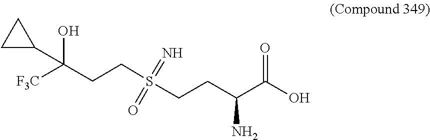 [SMILES: N=S(=O)(CC[C@H](N)C(=O)O)CCC(O)(C1CC1)C(F)(F)F]

### (2S)-2-amino-4-(3-cyclopropyl-4,4,4-trifluoro-3-hydroxybutylsulfonimidoyl)butanoic acid

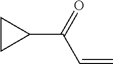

To a solution of N-methoxy-N-methylcyclopropanecarboxamide (2.00 g, 15.5 mmol, 1 eq) in THE (20 mL) was added vinylmagnesium chloride (2 M, 23.2 mL, 3 eq) at 0° C. The mixture was stirred at 0° C. for 3 h. The reaction mixture was diluted with MTBE (30 mL) and quenched by addition of water (30 mL) and the mixture was extracted with MTBE (30 mL×2). The combined organic layers were dried over Na₂SO₄ and filtered to obtain crude 1-cyclopropylprop-2-en-1-one (1.49 g, crude). The solution was used directly in the next step without further purification.

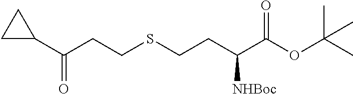 [SMILES: CC(C)(C)OC(=O)N[C@@H](CCSCCC(=O)C1CC1)C(=O)OC(C)(C)C]

To a solution of tert-butyl (tert-butoxycarbonyl)-L-homocysteinate (2.00 g, 6.86 mmol, 1 eq) in THE (20 mL) was added TEA (2.08 g, 20.6 mmol, 2.87 mL, 3 eq) and a solution of 1-cyclopropylprop-2-en-1-one (1.32 g, 13.7 mmol, 2 eq) in THE (20 mL)/MTBE (60 mL) under Ar. The mixture was stirred at 15° C. for 24 h before quenching with water (30 mL) and the mixture was extracted with EtOAc (50 mL×2). The combined organic layers were dried over Na₂SO₄, filtered, and concentrated under reduced pressure. The crude residue was purified by column chromatography (SiO₂, petroleum ether/EtOAc=100:0 to 95:5), followed by prep-HPLC (Welch Xtimate C18 (250×70 mm, 10 μm), 45-75% MeCN/water-NH₄HCO₃) to obtain (S)-tert-butyl 2-((tert-butoxycarbonyl)amino)-4-((3-cyclopropyl-3-oxopropyl)thio)butanoate (1.83 g, 4.21 mmol, 61.4% yield) as a yellow solid. ¹H NMR (400 MHz, CDCl₃-d) δ 5.04 (br d, 1H), 4.19 (br d, 1H), 2.84-2.75 (m, 2H), 2.73-2.65 (m, 2H), 2.48 (ddd, 2H), 2.09-1.95 (m, 1H), 1.91-1.75 (m, 2H), 1.39 (d, 18H), 0.98 (quin, 2H), 0.83 (dd, 2H)

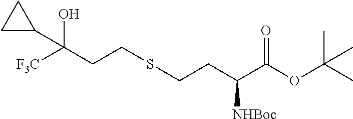 [SMILES: CC(C)(C)OC(=O)N[C@@H](CCSCCC(O)(C1CC1)C(F)(F)F)C(=O)OC(C)(C)C]

To a solution of compound (S)-tert-butyl 2-((tert-butoxycarbonyl)amino)-4-((3-cyclopropyl-3-oxopropyl)thio)butanoate (330 mg, 852 μmol, 1 eq) in THE (10 mL) was added TBAF (1 M, 170 μL, 0.2 eq) and TMSCF₃ (1.21 g, 8.52 mmol, 10 eq) and the mixture was stirred at 50° C. for 0.5 h. The mixture was cooled to 0° C. and TBAF (1 M, 2.55 mL, 3 eq) was added and the mixture was stirred at 20° C. for 1h. The reaction mixture was poured into water (15 mL) at 0° C. and extracted with EtOAc (30 mL×3). The combined organic phases were concentrated and the residue was purified by column chromatography (SiO₂, petroleum ether/EtOAc=100:0 to 90:10) to give the (2S)-tert-butyl 2-((tert-butoxycarbonyl)amino)-4-((3-cyclopropyl-4,4,4-trifluoro-3-hydroxybutyl)thio)butanoate (230 mg, 30% yield) as a yellow oil. ¹H NMR (400 MHz, CDCl₃-d) δ 5.07 (br d, 1H), 4.22 (br d, 1H), 2.90 (br s, 1H), 2.78-2.63 (m, 2H), 2.60-2.44 (m, 2H), 2.14-1.90 (m, 3H), 1.90-1.74 (m, 1H), 1.49-1.28 (m, 18H), 0.86 (dt, 1H), 0.61-0.51 (m, 1H), 0.49-0.35 (m, 3H).

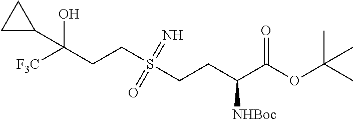 [SMILES: CC(C)(C)OC(=O)N[C@@H](CCS(=N)(=O)CCC(O)(C1CC1)C(F)(F)F)C(=O)OC(C)(C)C]

A mixture of (2S)-tert-butyl 2-((tert-butoxycarbonyl)amino)-4-((3-cyclopropyl-4,4,4-trifluoro-3-hydroxybutyl)thio)butanoate (230 mg, 503 μmol, 1 eq), PhI(OAc)₂ (486 mg, 1.51 mmol, 3 eq) and ammonium carbamate (196 mg, 2.51 mmol, 5 eq) in i-PrOH (10 mL) was stirred at 17° C. for 16 h. The reaction mixture was concentrated under reduced pressure and the residue was diluted with water (20 mL) and extracted with DCM (30 mL×3). The combined organic layers were dried over Na₂SO₄, filtered, and concentrated under reduced pressure. The crude residue was purified by column chromatography (SiO₂, petroleum ether/EtOAc=95:5 to 60:40) to give (2S)-tert-butyl 2-((tert-butoxycarbonyl)amino)-4-(3-cyclopropyl-4,4,4-trifluoro-3-hydroxybutylsulfonimidoyl)butanoate (210 mg, 430 μmol, 85.5% yield) as a colorless oil. ¹H NMR (400 MHz, CDCl₃-d) δ 5.16 (br d, 1H), 4.22 (br d, 1H), 3.57-3.36 (m, 1H), 3.33-2.97 (m, 3H), 2.61-2.49 (m, 1H), 2.41-2.24 (m, 2H), 2.21-2.12 (m, 1H), 2.10-2.00 (m, 1H), 1.50-1.30 (m, 18H), 0.92-0.74 (m, 1H), 0.69-0.55 (m, 1H), 0.54-0.35 (m, 3H).

A solution of (2S)-tert-butyl 2-((tert-butoxycarbonyl)amino)-4-(3-cyclopropyl-4,4,4-trifluoro-3-hydroxybutylsulfonimidoyl)butanoate (100 mg, 205 μmol, 1 eq) in HCl/dioxane (4 M, 10 mL, 195 eq) was stirred at 17° C. for 16 h. The reaction mixture was concentrated under reduced pressure and the residue was purified by prep-HPLC (C18-1 (150×30 mm, 5 μm), mobile phase: [H₂O (0.2% FA)-MeCN]; gradient: 5-35% B) to give (2S)-2-amino-4-(3-cyclopropyl-4,4,4-trifluoro-3-hydroxybutylsulfonimidoyl)butanoic acid (32.92 mg, 87.0 μmol, 42.5% yield) as a formic acid salt. LCMS: Rt=0.777 min, (ES⁺) m/z (M+H)⁺=333.0, HPLC Conditions: F. ¹H NMR (400 MHz, D₂O) δ 3.93-3.77 (m, 1H), 3.61-3.23 (m, 4H), 2.41-2.15 (m, 4H), 1.03 (br t, 1H), 0.59-0.34 (m, 4H).

### Exemplary Embodiment 1aa23

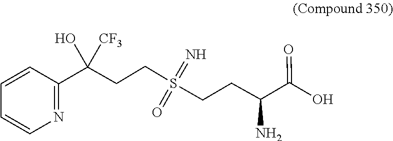 [SMILES: N=S(=O)(CC[C@H](N)C(=O)O)CCC(O)(c1ccccn1)C(F)(F)F]

### (2S)-2-amino-4-(4,4,4-trifluoro-3-hydroxy-3-(pyridin-2-yl)butylsulfonimidoyl)butanoic acid

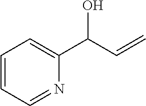

To a solution of picolinaldehyde (4 g, 37.3 mmol, 1 eq) in THE (40 mL) was added bromo(vinyl)magnesium (1 M, 41.1 mL, 1.1 eq) at 0° C. under N₂. The mixture was stirred at 0° C. for 1 h under N₂. The mixture was poured into saturated aqueous NH₄Cl solution (50 mL) and extracted with EtOAc (50 mL×3). The combined organic layers were dried over Na₂SO₄, filtered, and concentrated under reduced pressure. The residue was purified by column chromatography (SiO₂, petroleum ether/EtOAc=85:15) to give 1-(pyridin-2-yl)prop-2-en-1-ol (3.8 g, 28.1 mmol, 75.2% yield) as a yellow oil. ¹H NMR (400 MHz, CDCl₃-d) δ 8.56 (d, 1H), 7.71 (dt, 1H), 7.30 (d, 1H), 7.23 (dd, 1H), 5.97 (ddd, 1H), 5.47 (td, 1H), 5.34-5.14 (m, 2H), 4.91-4.42 (m, 1H).

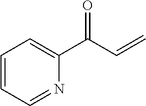

To a solution of (COCl)₂ (1.27 g, 9.99 mmol, 874 μL, 1.35 eq) in DCM (25 mL) was added dropwise DMSO (1.56 g, 19.9 mmol, 1.56 mL, 2.7 eq) at −65° C. under N₂. The mixture was stirred at −65° C. for 0.5 h under N₂. Then tert-butyl (tert-butoxycarbonyl)-L-homocysteinate (1.00 g, 7.40 mmol, 1 eq) in DCM (5 mL) was added dropwise and the mixture was stirred at −65° C. for 2 h under N₂. TEA (2.99 g, 29.5 mmol, 4.12 mL, 4 eq) was added at −65° C. for 0.5 h under N₂. The mixture was poured into water (20 mL) and extracted with EtOAc (15 mL×3). The combined organic layers were dried over Na₂SO₄, filtered, and concentrated under reduced pressure to give 1-(pyridin-2-yl)prop-2-en-1-one (1.1 g, crude) as a yellow oil. ¹H NMR (400 MHz, CDCl₃-d) δ 8.72 (br d, 1H), 8.14 (d, 1H), 7.89-7.84 (m, 2H), 7.49-7.47 (m, 1H), 6.62 (dd, 1H), 6.00-5.90 (m, 1H).

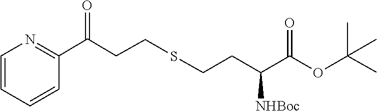 [SMILES: CC(C)(C)OC(=O)N[C@@H](CCSCCC(=O)c1ccccn1)C(=O)OC(C)(C)C]

To a solution of 1-(pyridin-2-yl)prop-2-en-1-one (980 mg, 7.36 mmol, 1 eq) and tert-butyl (tert-butoxycarbonyl)-L-homocysteinate (1.39 g, 4.78 mmol, 0.65 eq) in THE (10 mL) was added TEA (2.23 g, 22.8 mmol, 3.07 mL, 3 eq). The mixture was stirred at 25° C. for 16 h under N₂. The mixture was poured into water (10 mL) and extracted with EtOAc (15 mL×3). The combined organic layers were dried over Na₂SO₄, filtered, and concentrated under reduced pressure. The residue was purified by column chromatography (SiO₂, petroleum ether/EtOAc=88:12) to give tert-butyl N-(tert-butoxycarbonyl)-S-(3-oxo-3-(pyridin-2-yl)propyl)-L-homocysteinate (940 mg, 2.21 mmol, 30.1% yield) as a yellow oil. ¹H NMR (400 MHz, CDCl₃-d) δ 8.69 (d, 1H), 8.06 (d, 1H), 7.85 (dt, 1H), 7.49 (ddd, 1H), 5.14 (br d, 1H), 4.28 (br d, 1H), 3.61-3.45 (m, 2H), 3.01-2.87 (m, 2H), 2.71-2.53 (m, 2H), 2.21-2.04 (m, 1H), 1.98-1.85 (m, 1H), 1.48-1.44 (m, 18H).

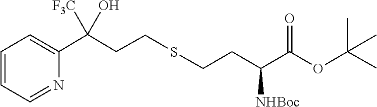 [SMILES: CC(C)(C)OC(=O)N[C@@H](CCSCCC(O)(c1ccccn1)C(F)(F)F)C(=O)OC(C)(C)C]

To a solution of tert-butyl N-(tert-butoxycarbonyl)-S-(3-oxo-3-(pyridin-2-yl)propyl)-L-homocysteinate (460 mg, 1.08 mmol, 1 eq) in THE (6 mL) was added TMSCF₃ (1.54 g, 10.8 mmol, 10 eq) and TBAF (1 M, 216 μL, 0.2 eq) at 0° C. The mixture was stirred at 25° C. for 0.5 h under N₂, then TBAF (1 M, 3.2 mL, 3 eq) was added at 0° C. The mixture was stirred at 25° C. for 0.5 h under N₂. The mixture was poured into water (10 mL) and extracted with EtOAc (15 mL×3). The combined organic layers were dried over Na₂SO₄, filtered, and concentrated under reduced pressure. The residue was purified by column chromatography (SiO₂, petroleum ether/EtOAc=92:8) to give tert-butyl N-(tert-butoxycarbonyl)-S-(4,4,4-trifluoro-3-hydroxy-3-(pyridin-2-yl)butyl)-L-homocysteinate (670 mg, 1.35 mmol, 62.5% yield) as a yellow oil. ¹H NMR (400 MHz, CDCl₃-d) δ 8.62 (d, 1H), 7.91-7.81 (m, 1H), 7.59-7.51 (m, 1H), 7.41 (dd, 1H), 6.53-6.17 (m, 1H), 5.16-4.95 (m, 1H), 4.22 (br d, 1H), 2.65-2.38 (m, 3H), 2.36-2.24 (m, 1H), 2.12-1.92 (m, 2H), 1.87-1.75 (m, 1H), 1.72-1.62 (m, 1H), 1.49-1.40 (m, 18H)

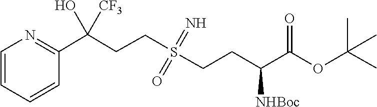 [SMILES: CC(C)(C)OC(=O)N[C@@H](CCS(=N)(=O)CCC(O)(c1ccccn1)C(F)(F)F)C(=O)OC(C)(C)C]

To a solution of tert-butyl N-(tert-butoxycarbonyl)-S-(4,4,4-trifluoro-3-hydroxy-3-(pyridin-2-yl)butyl)-L-homocysteinate (150 mg, 303 μmol, 1 eq) in i-PrOH (3 mL) was added PhI(OAc)₂ (390 mg, 1.21 mmol, 4 eq) and ammonium carbamate (189 mg, 2.43 mmol, 8 eq). The mixture was stirred at 25° C. for 16 h. The mixture was poured into water (10 mL) and extracted with EtOAc (15 mL×3). The combined organic layers were dried over Na₂SO₄, filtered, and concentrated under reduced pressure. The residue was purified by prep-TLC (SiO₂, petroleum ether/EtOAc=1:2, R_{f}=0.43) to give tert-butyl (2S)-2-((tert-butoxycarbonyl)amino)-4-(4,4,4-trifluoro-3-hydroxy-3-(pyridin-2-yl)butylsulfonimidoyl)butanoate (70 mg, 133 μmol, 43.9% yield) as a yellow oil. ¹H NMR (400 MHz, CDCl₃-d) δ 8.63 (dd, 1H), 7.88 (ddt, 1H), 7.63 (br t, 1H), 7.43 (dd, 1H), 5.22-5.09 (m, 1H), 4.35-4.19 (m, 1H), 3.26-2.92 (m, 3H), 2.81-2.54 (m, 3H), 2.44-2.27 (m, 1H), 2.16-2.06 (m, 1H), 1.46 (dd, 18H).

A mixture of tert-butyl (2S)-2-((tert-butoxycarbonyl)amino)-4-(4,4,4-trifluoro-3-hydroxy-3-(pyridin-2-yl)butylsulfonimidoyl)butanoate (70 mg, 133 μmol, 1 eq) in HCl/dioxane (4 M, 5 mL, 150 eq) was stirred at 25° C. for 16 h. The reaction mixture was concentrated under reduced pressure and the residue was purified by prep-HPLC (column: Phenomenex Luna C18 (200×40 mm, 10 μm); mobile phase: [water(FA)-ACN]; gradient: 1-25% B, 8 min) to give (2S)-2-amino-4-(4,4,4-trifluoro-3-hydroxy-3-(pyridin-2-yl)butylsulfonimidoyl)butanoic acid (16 mg, 39.9 μmol, 29.9% yield) as a white solid. LCMS: Rt=1.196 min, (ES⁺) m/z (M+H)⁺=370.0, HPLC Conditions: C. ¹H NMR (400 MHz, D₂O) δ 8.57 (d, 1H), 7.96 (dt, 1H), 7.78 (d, 1H), 7.49 (ddd, 1H), 3.84-3.83 (m, 1H), 3.44-3.17 (m, 3H), 2.91-2.77 (m, 2H), 2.66-2.51 (m, 1H), 2.32-2.17 (m, 2H).

### Exemplary Embodiment 1aa24

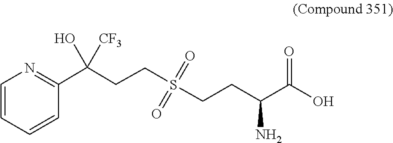 [SMILES: N[C@@H](CCS(=O)(=O)CCC(O)(c1ccccn1)C(F)(F)F)C(=O)O]

### (2S)-2-amino-4-((4,4,4-trifluoro-3-hydroxy-3-(pyridin-2-yl)butyl)sulfonyl)butanoic acid

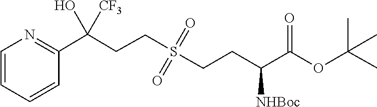 [SMILES: CC(C)(C)OC(=O)N[C@@H](CCS(=O)(=O)CCC(O)(c1ccccn1)C(F)(F)F)C(=O)OC(C)(C)C]

To a solution of tert-butyl N-(tert-butoxycarbonyl)-S-(4,4,4-trifluoro-3-hydroxy-3-(pyridin-2-yl)butyl)-L-homocysteinate (74 mg, 149 μmol, 1 eq) in DCM (2 mL) was added m-CPBA (80.6 mg, 374 μmol, 80% purity, 2.5 eq) at 0° C. under N₂. The mixture was stirred at 20° C. for 1 hr under N₂. The mixture was poured into water (5 mL) and extracted with EtOAc (5 mL×2). The combined organic layers were dried over Na₂SO₄, filtered, and concentrated under reduced pressure. The residue was purified by prep-TLC (SiO₂, petroleum ether/EtOAc=2:1, R_{f}=0.43) to give tert-butyl (2S)-2-((tert-butoxycarbonyl)amino)-4-((4,4,4-trifluoro-3-hydroxy-3-(pyridin-2-yl)butyl)sulfonyl)butanoate (71 mg, 134 μmol, 90.1% yield) as a yellow oil. ¹H NMR (400 MHz, CDCl₃-d) δ 8.64 (d, 1H), 7.90 (tt, 1H), 7.59 (d, 1H), 7.45 (dd, 1H), 6.43 (br d, 1H), 5.15 (br s, 1H), 4.23 (br d, 1H), 3.22-2.89 (m, 3H), 2.78-2.65 (m, 1H), 2.64-2.45 (m, 2H), 2.41-2.28 (m, 1H), 2.13-2.06 (m, 1H), 1.47 (d, 18H).

A solution of tert-butyl (2S)-2-((tert-butoxycarbonyl)amino)-4-((4,4,4-trifluoro-3-hydroxy-3-(pyridin-2-yl)butyl)sulfonyl)butanoate (71 mg, 134 μmol, 1 eq) in HCl/dioxane (5 M, 6 mL, 222 eq) was stirred at 30° C. for 16 h. The reaction mixture was concentrated under reduced pressure and the residue was purified by prep-HPLC (column: Phenomenex Luna C18 (100×40 mm, 3 μm); mobile phase: [H₂O (0.2% FA)-MeCN]; gradient: 1-40% B over 8.0 min) to give (2S′)-2-amino-4-((4,4,4-trifluoro-3-hydroxy-3-(pyridin-2-yl)butyl)sulfonyl)butanoic acid (15 mg, 40.5 mol, 30.0% yield) as a white solid. LCMS: Rt=1.508 min (ES⁺) m/z (M+H)⁺=371.0. ¹H NMR (400 MHz, MeOD-d₄) δ 8.64 (dd, 1H), 7.96-7.87 (m, 1H), 7.80 (d, 1H), 7.45 (ddd, 1H), 3.68 (dt, 1H), 3.40-3.32 (m, 1H), 3.28-3.08 (m, 2H), 2.91 (dt, 1H), 2.74-2.60 (m, 1H), 2.59-2.49 (m, 1H), 2.35-2.17 (in, 2H).

The compounds described in Table 14 were prepared using the general methods

TABLE 14
Characterization of Compounds 352-380

| Compound | | MS | HPLC | HPLC |
|---|---|---|---|---|
| # | Structure | (M + H)⁺ | Rt | cond. |
| 352 | 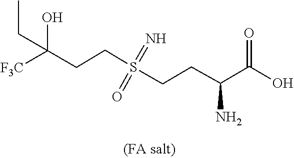 [SMILES: CCC(O)(CCS(=N)(=O)CC[C@H](N)C(=O)O)C(F)(F)F] | 321.0 | 1.61 | A |
| | ¹H NMR (400 MHz, D₂O) δ 3.98-3.79 (m, 1H), | | | |
| | 3.55-3.30 (m, 4H), 2.41-2.29 (m, 2H), 2.23-2.14 | | | |
| | (m, 2H), 1.78 (td, 2H), 0.95 (t, 3H) | | | |
| 353 | 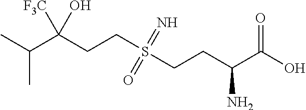 [SMILES: CC(C)C(O)(CCS(=N)(=O)CC[C@H](N)C(=O)O)C(F)(F)F] | 335.1 | 1.35 | C |
| | ¹H NMR (400 MHz, D₂O) δ 3.96 (t, 1H), 3.69- | | | |
| | 3.46 (m, 4H), 2.40 (q, 2H), 2.28-2.19 (m, 2H), | | | |
| | 2.13 (td, 1H), 1.01 (br t6H). | | | |
| 354 | 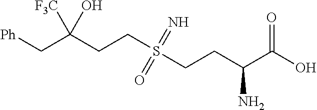 [SMILES: N=S(=O)(CC[C@H](N)C(=O)O)CCC(O)(Cc1ccccc1)C(F)(F)F] | 383.1 | 1.73 | C |
| | ¹H NMR (400 MHz, DMSO-d6) δ 7.74-7.42 (m, | | | |
| | 2H), 7.37-7.21 (m, 5H), 6.72-6.50 (m, 1H), 3.94- | | | |
| | 3.75 (m, 1H), 3.28-3.15 (m, 2H), 3.11-2.84 (m, | | | |
| | 5H), 2.12-1.89 (m, 4H) | | | |
| 355 | 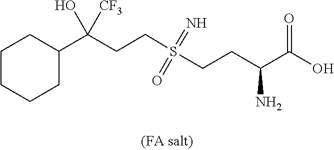 [SMILES: N=S(=O)(CC[C@H](N)C(=O)O)CCC(O)(C1CCCCC1)C(F)(F)F] | 375.1 | 1.91 | C |
| | ¹H NMR (400 MHz, D₂O) δ 3.96-3.79 (m, 1H), | | | |
| | 3.57-3.25 (m, 4H), 2.43-2.30 (m, 2H), 2.25-2.16 | | | |
| | (m, 2H), 1.86-1.69 (m, 5H), 1.67-1.59 (m, 1H), | | | |
| | 1.25-1.08 (m, 5H) | | | |
| 356 | 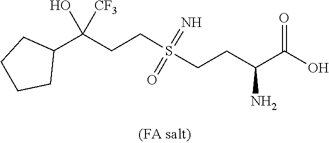 [SMILES: N=S(=O)(CC[C@H](N)C(=O)O)CCC(O)(C1CCCC1)C(F)(F)F] | 361.1 | 2.75 | A |
| | ¹H NMR (400 MHz, D₂O) δ 3.95-3.78 (m, 1H), | | | |
| | 3.55-3.28 (m, 4H), 2.43-2.11 (m, 5H), 1.73 (br d, | | | |
| | 2H), 1.66-1.35 (m, 6H) | | | |
| 357 | 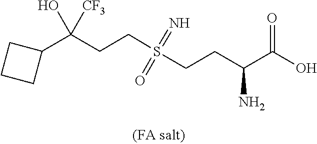 [SMILES: N=S(=O)(CC[C@H](N)C(=O)O)CCC(O)(C1CCC1)C(F)(F)F] | 347.0 | 1.51 | C |
| | ¹H NMR (400 MHz, D₂O) δ 3.91-3.81 (m, 1H), | | | |
| | 3.52-3.31 (m, 4H), 2.86-2.72 (m, 1H), 2.41-2.27 | | | |
| | (m, 2H), 2.14-1.99 (m, 4H), 1.94-1.82 (m, 3H), | | | |
| | 1.66 (q, 1H) | | | |
| 358 | 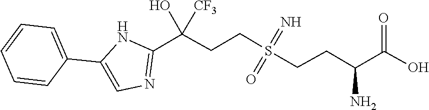 [SMILES: N=S(=O)(CC[C@H](N)C(=O)O)CCC(O)(c1ncc(-c2ccccc2)[nH]1)C(F)(F)F] | 435.1 | 1.52 | C |
| | ¹H NMR (400 MHz, D₂O) δ 7.70 (d, 2H), 7.54 (s, | | | |
| | 1H), 7.47 (t, 2H), 7.39-7.34 (m, 1H), 3.88-3.78 | | | |
| | (m, 1H), 3.53-3.24 (m, 4H), 3.18-3.04 (m, 1H), | | | |
| | 2.91-2.79 (m, 1H), 2.70-2.56 (m, 1H), 2.37-2.27 | | | |
| | (m, 2H). | | | |
| 359 | 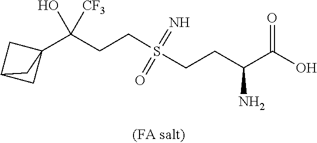 [SMILES: N=S(=O)(CC[C@H](N)C(=O)O)CCC(O)(C(F)(F)F)C12CC(C1)C2] | 359.1 | 1.89 | B |
| | ¹H NMR (400 MHz, D₂O) δ 8.41 (s, 1H), 3.90- | | | |
| | 3.81 (m, 1H), 3.53-3.29 (m, 4H), 2.48 (s, 1H), | | | |
| | 2.41-2.29 (m, 2H), 2.28-2.07 (m, 2H), 1.89 (s, | | | |
| | 6H) | | | |
| 360 | 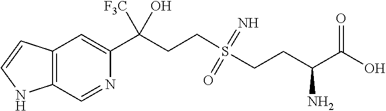 [SMILES: N=S(=O)(CC[C@H](N)C(=O)O)CCC(O)(c1cc2cc[nH]c2cn1)C(F)(F)F] | 409.1 | 1.78 | B |
| | ¹H NMR (400 MHz, D₂O) δ 8.75 (s, 1H), 8.01 (s, | | | |
| | 1H), 7.65 (br s, 1H), 6.67 (br s, 1H), 3.86-3.72 (m, | | | |
| | 1H), 3.44-3.15 (m, 3H), 2.99-2.73 (m, 2H), 2.66- | | | |
| | 2.51 (m, 1H), 2.31-2.10 (m, 2H). | | | |
| 361 | 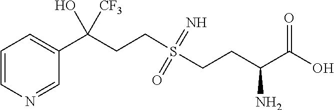 [SMILES: N=S(=O)(CC[C@H](N)C(=O)O)CCC(O)(c1cccnc1)C(F)(F)F] | 370.1 | 2.64 | G |
| | ¹H NMR (400 MHz, D₂O) δ 8.70 (s, 1H), 8.52 (d, | | | |
| | 1H), 8.02 (br d, 1H), 7.50 (dd, 1H), 3.77 (dt, 1H), | | | |
| | 3.43-3.17 (m, 3H), 2.99-2.86 (m, 1H), 2.78-2.54 | | | |
| | (m, 2H), 2.28-2.16 (m, 2H) | | | |
| 362 | 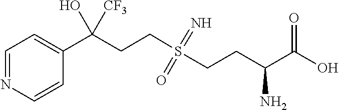 [SMILES: N=S(=O)(CC[C@H](N)C(=O)O)CCC(O)(c1ccncc1)C(F)(F)F] | 370.1 | 2.57 | G |
| | ¹H NMR (400 MHz, D₂O) δ 8.61 (d, 2H), 7.65 (d, | | | |
| | 2H), 3.85-3.78 (m, 1H), 3.46-3.24 (m, 3H), 2.93- | | | |
| | 2.80 (m, 1H), 2.77-2.59 (m, 2H), 2.34-2.17 (m, | | | |
| | 2H) | | | |
| 363 | 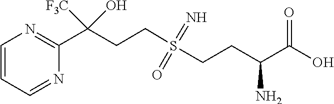 [SMILES: N=S(=O)(CC[C@H](N)C(=O)O)CCC(O)(c1ncccn1)C(F)(F)F] | 371.1 | 1.75 | A |
| | ¹H NMR (400 MHz, D₂O) δ 8.85 (d, 2H), 7.57 (t, | | | |
| | 1H), 3.84-3.73 (m, 1H), 3.46-3.19 (m, 3H), 2.95- | | | |
| | 2.76 (m, 2H), 2.67-2.52 (m, 1H), 2.24 (br dd, | | | |
| | 2H). | | | |
| 364 | 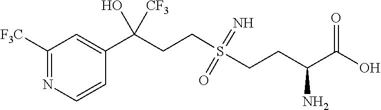 [SMILES: N=S(=O)(CC[C@H](N)C(=O)O)CCC(O)(c1ccnc(C(F)(F)F)c1)C(F)(F)F] | 438.1 | 2.06 | B |
| | ¹H NMR (400 MHz, D₂O) δ 8.77 (d, 1H), 8.11 (s, | | | |
| | 1H), 7.92 (br d, 1H), 3.91-3.76 (m, 1H), 3.48- | | | |
| | 3.18 (m, 3H), 3.00-2.65 (m, 3H), 2.36-2.13 (m, | | | |
| | 2H). | | | |
| 365 | 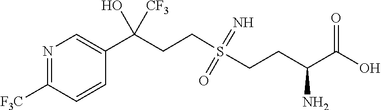 [SMILES: N=S(=O)(CC[C@H](N)C(=O)O)CCC(O)(c1ccc(C(F)(F)F)nc1)C(F)(F)F] | 438.1 | 2.10 | B |
| | ¹H NMR (400 MHz, MeOD-d₄) δ 8.97 (s, 1H), 8.30 | | | |
| | (br d, 1H), 7.91 (d, 1H), 3.77-3.61 (m, 1H), 3.36 | | | |
| | (br s, 1H), 3.28-3.14 (m, 2H), 2.92-2.58 (m, 3H), | | | |
| | 2.33-2.22 (m, 2H) | | | |
| 366 | 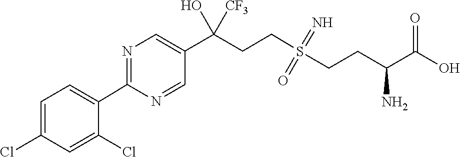 [SMILES: N=S(=O)(CC[C@H](N)C(=O)O)CCC(O)(c1cnc(-c2ccc(Cl)cc2Cl)nc1)C(F)(F)F] | 515.0 | 2.02 | C |
| | ¹H NMR (400 MHz, MeOD-d₄) δ 9.14 (s, 2H), 7.80 | | | |
| | (d, 1H), 7.64 (d, 1H), 7.50 (dd, 1H), 3.75-3.64 (m, | | | |
| | 1H), 3.47-3.33 (m, 2H), 3.29 (br s, 1H), 2.99- | | | |
| | 2.81 (m, 2H), 2.78-2.64 (m, 1H), 2.37-2.24 (m, | | | |
| | 2H). | | | |
| 367 | 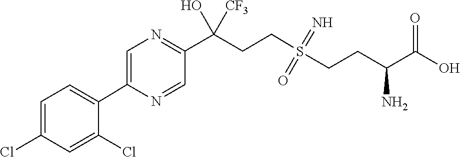 [SMILES: N=S(=O)(CC[C@H](N)C(=O)O)CCC(O)(c1cnc(-c2ccc(Cl)cc2Cl)cn1)C(F)(F)F] | 515.1 | 2.09 | C |
| | 1H NMR (400 MHz, MeOD-d₄) δ 9.10 (s, 1H), | | | |
| | 9.00 (s, 1H), 7.74-7.66 (m, 2H), 7.53 (dd, 1H), | | | |
| | 4.60 (s, 2H), 3.79-3.57 (m, 1H), 3.35 (s, 1H), 3.27- | | | |
| | 3.11 (m, 2H), 3.10-3.02 (m, 1H), 2.99-2.84 (m, | | | |
| | 1H), 2.69-2.54 (m, 1H), 2.35-2.13 (m, 2H) | | | |
| 368 | 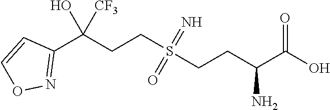 [SMILES: N=S(=O)(CC[C@H](N)C(=O)O)CCC(O)(c1ccon1)C(F)(F)F] | 360.0 | 0.988 | B |
| | ¹H NMR (400 MHz, MeOD-d₄) δ 8.77 (d, 1H), 6.68 | | | |
| | (s, 1H), 4.18 (dq, 1H), 3.59-3.37 (m, 3H), 3.26- | | | |
| | 3.09 (m, 1H), 2.84-2.71 (m, 1H), 2.64-2.33 (m, | | | |
| | 3H) | | | |
| 369 | 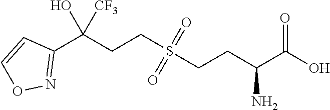 [SMILES: N[C@@H](CCS(=O)(=O)CCC(O)(c1ccon1)C(F)(F)F)C(=O)O] | 361.0 | 1.30 | C |
| | ¹H NMR (400 MHz, D₂O) δ 8.73 (d, 1H), 6.72 (s, | | | |
| | 1H), 3.85 (t, 1H), 3.46-3.33 (m, 3H), 3.19-3.08 | | | |
| | (m, 1H), 2.73-2.57 (m, 2H), 2.37-2.26 (m, 2H) | | | |
| 370 | 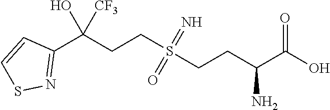 [SMILES: N=S(=O)(CC[C@H](N)C(=O)O)CCC(O)(c1ccsn1)C(F)(F)F] | 376.0 | 2.61 | G |
| | ¹H NMR (400 MHz, D₂O) δ 8.98 (d, 1H), 7.52 (d, | | | |
| | 1H), 3.76 (q, 1H), 3.44-3.22 (m, 3H), 3.00-2.83 | | | |
| | (m, 1H), 2.77 (dt, 1H), 2.62-2.47 (m, 1H), 2.23 (br | | | |
| | dd, 2H). | | | |
| 371 | 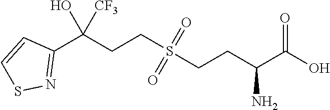 [SMILES: N[C@@H](CCS(=O)(=O)CCC(O)(c1ccsn1)C(F)(F)F)C(=O)O] | 377.0 | 1.66 | B |
| | ¹H NMR (400 MHz, D₂O) δ 8.98 (d, 1H), 7.52 (d, | | | |
| | 1H), 3.79 (br t, 1H), 3.42-3.17 (m, 3H), 2.99- | | | |
| | 2.86 (m, 1H), 2.78 (dt, 1H), 2.62-2.50 (m, 1H), | | | |
| | 2.24 (q, 2H) | | | |
| 372 | 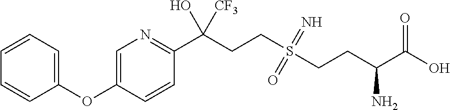 [SMILES: N=S(=O)(CC[C@H](N)C(=O)O)CCC(O)(c1ccc(Oc2ccccc2)cn1)C(F)(F)F] | 462.1 | 2.32 | B |
| | ¹H NMR (400 MHz, MeOD-d₄) δ 8.38 (br s, 1H), | | | |
| | 7.79 (br d, 1H), 7.46 (br t, 3H), 7.30-7.21 (m, 1H), | | | |
| | 7.13 (br d, 2H), 3.77-3.62 (m, 1H), 3.45-3.37 (m, | | | |
| | 1H), 3.30-3.13 (m, 2H), 3.03-2.90 (m, 1H), 2.88- | | | |
| | 2.74 (m, 1H), 2.62-2.48 (m, 1H), 2.39-2.18 (m, | | | |
| | 2H). | | | |
| 373 | 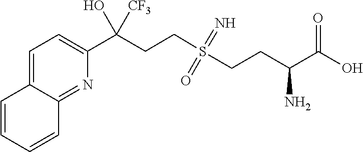 [SMILES: N=S(=O)(CC[C@H](N)C(=O)O)CCC(O)(c1ccc2ccccc2n1)C(F)(F)F] | 420.0 | 2.19 | B |
| | ¹H NMR (400 MHz, D₂O) δ 8.48 (d, 1H), 8.10 (br | | | |
| | d, 1H), 8.00 (br d, 1H), 7.90-7.79 (m, 2H), 7.67 (s, | | | |
| | 1H), 3.77 (br d, 1H), 3.50-3.18 (m, 3H), 3.01- | | | |
| | 2.63 (m, 3H), 2.29-2.14 (m, 2H) | | | |
| 374 | 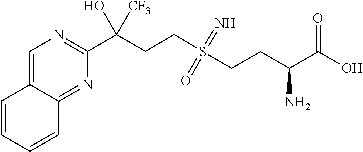 [SMILES: N=S(=O)(CC[C@H](N)C(=O)O)CCC(O)(c1ncc2ccccc2n1)C(F)(F)F] | 421.1 | 1.65 | C |
| | ¹H NMR (400 MHz, MeOD-d₄) δ 9.66 (s, 1H), 8.23- | | | |
| | 8.08 (m, 3H), 7.85 (t, 1H), 3.73-3.61 (m, 1H), | | | |
| | 3.43-3.34 (m, 1H), 3.28-3.20 (m, 2H), 3.18-3.08 | | | |
| | (m, 1H), 2.90-2.77 (m, 1H), 2.76-2.62 (m, 1H), | | | |
| | 2.41-2.19 (m, 2H). | | | |
| 375 | 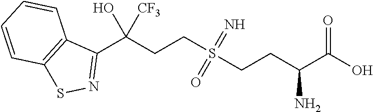 [SMILES: N=S(=O)(CC[C@H](N)C(=O)O)CCC(O)(c1nsc2ccccc12)C(F)(F)F] | 426.1 | 1.86 | C |
| | ¹H NMR (400 MHz, MeOD-d₄) δ 8.68 (d, 1H), 8.08 | | | |
| | (d, 1H), 7.59 (t, 1H), 7.53-7.42 (m, 1H), 3.73 (br | | | |
| | d, 1H), 3.55-3.35 (m, 2H), 3.31-3.23 (m, 2H), | | | |
| | 3.23-3.11 (m, 1H), 2.74-2.56 (m, 1H), 2.35 (br s, | | | |
| | 2H) | | | |
| 376 | 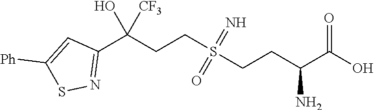 [SMILES: N=S(=O)(CC[C@H](N)C(=O)O)CCC(O)(c1cc(-c2ccccc2)sn1)C(F)(F)F] | 452.1 | 1.98 | C |
| | ¹H NMR (400 MHz, MeOD-d₄) δ 7.79 (s, 1H), 7.71 | | | |
| | (dd, 2H), 7.53-7.42 (m, 3H), 3.73-3.64 (m, 1H), | | | |
| | 3.46-3.34 (m, 2H), 3.30-3.20 (m, 1H), 3.07-2.90 | | | |
| | (m, 2H), 2.63-2.50 (m, 1H), 2.41-2.22 (m, 2H) | | | |
| 377 | 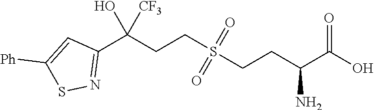 [SMILES: N[C@@H](CCS(=O)(=O)CCC(O)(c1cc(-c2ccccc2)sn1)C(F)(F)F)C(=O)O] | 453.1 | 2.10 | C |
| | ¹H NMR (400 MHz, MeOD-d₄) δ 7.79 (s, 1H), 7.71 | | | |
| | (dd, 2H), 7.54-7.43 (m, 3H), 3.69 (dt, 1H), 3.45- | | | |
| | 3.33 (m, 2H), 3.28-3.19 (m, 1H), 3.00-2.86 (m, | | | |
| | 2H), 2.59-2.47 (m, 1H), 2.36-2.23 (m, 2H) | | | |
| 378 | 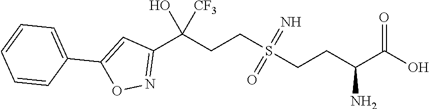 [SMILES: N=S(=O)(CC[C@H](N)C(=O)O)CCC(O)(c1cc(-c2ccccc2)on1)C(F)(F)F] | 436.1 | 1.93 | C |
| | ¹H NMR (400 MHz, MeOD-d₄) δ 7.86 (dd, 2H), | | | |
| | 7.56-7.49 (m, 3H), 6.97 (s, 1H), 3.75-3.66 (m, | | | |
| | 1H), 3.51-3.32 (m, 3H), 3.19-3.10 (m, 1H), 2.83- | | | |
| | 2.73 (m, 1H), 2.66-2.54 (m, 1H), 2.40-2.27 (m, | | | |
| | 2H) | | | |
| 379 | 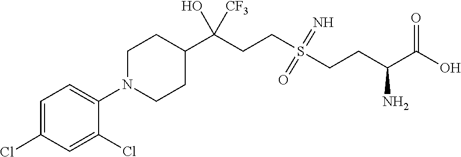 [SMILES: N=S(=O)(CC[C@H](N)C(=O)O)CCC(O)(C1CCN(c2ccc(Cl)cc2Cl)CC1)C(F)(F)F] | 520.1 | 2.19 | C |
| | ¹H NMR (400 MHz, MeOD-d₄) δ 7.39 (d, 1H), 7.27- | | | |
| | 7.23 (m, 1H), 7.10 (d, 1H), 3.77-3.67 (m, 1H), | | | |
| | 3.50-3.33 (m, 6H), 2.69-2.58 (m, 2H), 2.41-2.33 | | | |
| | (m, 2H), 2.30-2.22 (m, 2H), 1.96-1.87 (m, 3H), | | | |
| | 1.80-1.69 (m, 2H) | | | |
| 380 | 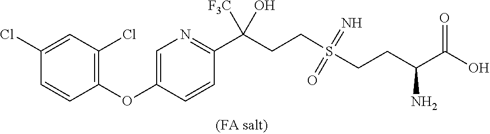 [SMILES: N=S(=O)(CC[C@H](N)C(=O)O)CCC(O)(c1ccc(Oc2ccc(Cl)cc2Cl)cn1)C(F)(F)F] | 530.1 | 2.23 | C |
| | ¹H NMR (400 MHz, MeOD-d₄) δ 8.38 (br s, 1H), | | | |
| | 8.28-8.22 (m, 1H), 7.81 (d, 1H), 7.67 (d, 1H), 7.49- | | | |
| | 7.37 (m, 2H), 7.23 (d, 1H), 3.72 (td, 1H), 3.46- | | | |
| | 3.35 (m, 1H), 3.32-3.25 (m, 1H), 3.24-3.13 (m, | | | |
| | 1H), 3.05-2.92 (m, 1H), 2.90-2.75 (m, 1H), 2.64- | | | |
| | 2.48 (m, 1H), 2.42-2.21 (m, 2H). | | | |

### Exemplary Embodiment 1aa25

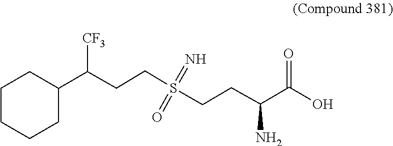 [SMILES: N=S(=O)(CCC(C1CCCCC1)C(F)(F)F)CC[C@H](N)C(=O)O]

### (2S′)-2-amino-4-(3-cyclohexyl-4,4,4-trifluorobutylsulfonimidoyl)butanoic acid

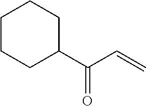

To a solution of N-methoxy-N-methylcyclohexanecarboxamide (2.00 g, 11.7 mmol, 1 eq) in THE (20 mL) was added vinylmagnesium bromide (1 M, 15.2 mL, 1.3 eq) at 0° C. and the mixture was stirred at 17° C. for 2 h. The reaction mixture was quenched by aq. NH₄Cl (20 mL), extracted with EtOAc (10 mL×2), and the combined organic layers were washed with brine (20 mL), dried over NaS₂O₄, filtered, and concentrated under reduced pressure to give 1-cyclohexylprop-2-en-1-one (1.60 g, 11.6 mmol, 99.1% yield) as a yellow oil and used directly.

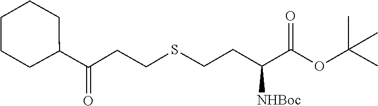 [SMILES: CC(C)(C)OC(=O)N[C@@H](CCSCCC(=O)C1CCCCC1)C(=O)OC(C)(C)C]

To a mixture of (S)-tert-butyl 2-((tert-butoxycarbonyl)amino)-4-mercaptobutanoate (1.60 g, 5.49 mmol, 1 eq) and TEA (1.39 g, 13.7 mmol, 1.91 mL, 2.5 eq) in THE (20 mL) was added tert-butyl (tert-butoxycarbonyl)-L-homocysteinate (1.52 g, 10.9 mmol, 2 eq) under N₂ atmosphere and the mixture was stirred at 17° C. for 16 h. The reaction mixture was concentrated under reduced pressure and the residue was purified by column chromatography (SiO₂, petroleum ether/EtOAc=1:0 to 1:1) to give (S)-tert-butyl 2-((tert-butoxycarbonyl)amino)-4-((3-cyclohexyl-3-oxopropyl)thio)butanoate (1.70 g, 3.96 mmol, 72.07% yield) as a yellow oil. ¹H NMR (400 MHz, CDCl₃-d) δ 5.10 (br d, 1H), 4.26 (br d, 1H), 2.56-2.52 (m, 2H), 2.43-2.32 (m, 2H), 2.11-2.03 (m, 1H), 1.92-1.74 (m, 12H), 1.68 (br d, 2H), 1.47 (s, 9H), 1.45 (s, 9H).

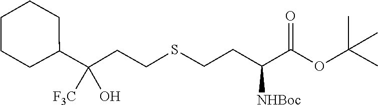 [SMILES: CC(C)(C)OC(=O)N[C@@H](CCSCCC(O)(C1CCCCC1)C(F)(F)F)C(=O)OC(C)(C)C]

To a solution of (S)-tert-butyl 2-((tert-butoxycarbonyl)amino)-4-((3-cyclohexyl-3-oxopropyl)thio)butanoate (480 mg, 1.12 mmol, 1 eq) in THE (8 mL) was added TMSCF₃ (477 mg, 3.35 mmol, 3 eq) and TBAF (1 M, 111.73 μL, 0.1 eq) at 0° C. and the mixture was stirred at 20° C. for 1 h under N₂. Then TBAF (1 M, 3.35 mL, 3 eq) was added and the mixture was stirred at 20° C. for 1 h under N₂. The reaction mixture was poured into water (30 mL), extracted with EtOAc (20 mL×2), and the combined organic layers were washed with brine (30 mL), dried over Na₂SO₄, filtered, and concentrated under reduced pressure. The residue was purified by column chromatography (SiO₂, petroleum ether/EtOAc=1:0 to 3:1) to give (2S)-tert-butyl 2-((tert-butoxycarbonyl)amino)-4-((3-cyclohexyl-4,4,4-trifluoro-3-hydroxybutyl)thio)butanoate (500 mg, 1.00 mmol, 89.6% yield) as a yellow oil. ¹H NMR (400 MHz, CDCl₃-d) δ 5.20-5.07 (m, 1H), 4.37-4.21 (m, 1H), 2.73-2.64 (m, 2H), 2.63-2.56 (m, 2H), 2.05 (s, 1H), 1.99-1.67 (m, 10H), 1.48 (s, 9H), 1.45 (s, 9H), 1.31-1.21 (m, 4H).

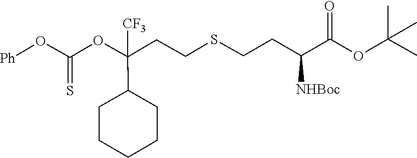 [SMILES: CC(C)(C)OC(=O)N[C@@H](CCSCCC(OC(=S)Oc1ccccc1)(C1CCCCC1)C(F)(F)F)C(=O)OC(C)(C)C]

To a solution of (2S)-tert-butyl 2-((tert-butoxycarbonyl)amino)-4-((3-cyclohexyl-4,4,4-trifluoro-3-hydroxybutyl)thio)butanoate (500 mg, 1.00 mmol, 1 eq) in THE (10 mL) was added KHMDS (1 M, 3.60 mL, 3.6 eq) at −65° C. under N₂, then the mixture was stirred at −20° C. for 1 h. Ortho-phenyl chlorothionoformate (864 mg, 5.00 mmol, 691 μL, 5 eq) was added at −20° C. and the mixture was stirred at 0° C. for 1 h. The reaction mixture was poured into aq. NaHCO₃ (15 mL), extracted with EtOAc (10 mL×2), and the combined organic layers were washed with brine (15 mL), dried over Na₂SO₄, filtered, and concentrated under reduced pressure. The crude residue was purified by column chromatography (SiO₂, petroleum ether/EtOAc=98:2 to 85:15) to give (2S)-tert-butyl 2-((tert-butoxycarbonyl)amino)-4-((3-cyclohexyl-4,4,4-trifluoro-3-((phenoxycarbonothioyl)oxy)butyl)thio)butanoate (260 mg, 409 μmol, 40.9% yield) as a yellow oil. ¹H NMR (400 MHz, CDCl₃-d) δ 7.44 (br d, 2H), 7.34-7.29 (m, 1H), 7.08 (br d, 2H), 5.10 (br d, 1H), 4.29 (br d, 1H), 3.03-2.83 (m, 2H), 2.78-2.68 (m, 2H), 2.65-2.56 (m, 2H), 2.40-2.30 (m, 1H), 2.09 (br d, 1H), 1.94-1.60 (m, 8H), 1.58-1.50 (m, 3H), 1.50-1.46 (m, 9H), 1.45 (s, 9H).

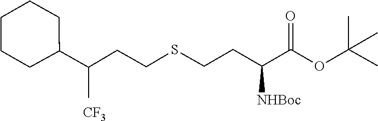

To a solution of (2S)-tert-butyl 2-((tert-butoxycarbonyl)amino)-4-((3-cyclohexyl-4,4,4-trifluoro-3-((phenoxycarbonothioyl)oxy)butyl)thio)butanoate (260 mg, 409 μmol, 1 eq) in toluene (3 mL) was added Bu₃SnH (476 mg, 1.64 mmol, 433 μL, 4 eq) and AIBN (26.9 mg, 163 μmol, 0.4 eq) under N₂. The mixture was stirred at 100° C. for 1 h before quenching with aq. KF (10 mL). The mixture was extracted with EtOAc (5 mL×2) and the combined organic layers were washed with brine (10 mL), dried over Na₂SO₄, filtered, and concentrated under reduced pressure to give a residue. The residue was purified by column chromatography (SiO₂, petroleum ether/EtOAc=1:0 to 3:1) to give (115 mg, 238 μmol, 58.1% yield) as a yellow oil. ¹H NMR (400 MHz, CDCl₃-d) δ 5.10 (br d, 1H), 4.28 (br d, 1H), 2.68-2.63 (m, 1H), 2.60-2.49 (m, 4H), 2.09 (br d, 1H), 1.86 (br dd, 2H), 1.70 (br s, 4H), 1.48 (s, 9H), 1.45 (s, 9H), 1.34-1.13 (m, 8H).

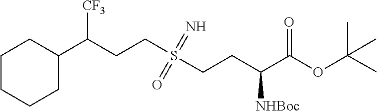 [SMILES: CC(C)(C)OC(=O)N[C@@H](CCS(=N)(=O)CCC(C1CCCCC1)C(F)(F)F)C(=O)OC(C)(C)C]

A mixture of (2S)-tert-butyl 2-((tert-butoxycarbonyl)amino)-4-((3-cyclohexyl-4,4,4-trifluorobutyl)thio)butanoate (115 mg, 238 μmol, 1 eq), ammonium carbamate (148 mg, 1.90 mmol, 8 eq), and PhI(OAc)₂ (306 mg, 951 μmol, 4 eq) in i-PrOH (1.5 mL) was stirred at 25° C. for 2 h. The reaction mixture was concentrated under reduced pressure and the residue was diluted with water (5 mL), extracted with EtOAc (3 mL×2), and the combined organic layers were washed with brine (5 mL), dried over Na₂SO₄, filtered, and concentrated under reduced pressure. The residue was purified by prep-TLC (SiO₂, petroleum ether/EtOAc=1:1) to give (2S′)-tert-butyl 2-((tert-butoxycarbonyl)amino)-4-(3-cyclohexyl-4,4,4-trifluorobutylsulfonimidoyl)butanoate (80 mg, 155 mol, 65.400 yield) as a yellow oil. ¹H NNR (400 MHz, CDCD3-d) δ 5.28-5.19 (m, 1H), 4.37-4.23 (m, 1H), 3.29-3.17 (, 2H), 3.13-3.05 (i, 1H), 2.44-2.35 (i, 1H), 2.23-2.05 (i, 4H), 1.76 (br t, 4H), 1.64 (br d, 2H), 1.49 (s, 9H), 1.46 (s, 9H), 1.34-1.15 (m, 6H).

A mixture of (2S)-tert-butyl 2-((tert-butoxycarbonyl)amino)-4-(3-cyclohexyl-4,4,4-trifluorobutylsulfonimidoyl)butanoate (80 mg, 155 mol, 1 eq) in HCl/dioxane (20 mL) (6M) was stirred at 17° C. for 12 h. The reaction mixture was concentrated under reduced pressure and the residue was purified by prep-HPLC (C18-1 (150×30 mm, 5 μm), mobile phase: [water (FA)-MeCN]; gradient: 15-45% B) to give (2S)-2-amino-4-(3-cyclohexyl-4,4,4-trifluorobutylsulfonimidoyl)butanoic acid (23.6 mg, 64.9 mol, 41.8% yield) as a white solid. LCMS: Rt=2.030 min.; (ES⁺) m/z (M+H)⁺=359.1, HPLC Conditions: C. ¹H NMR (400 3 MHz, D₂O) δ 3.85-3.79 (i, 1H), 3.47-3.22 (m, 4H), 2.35-2.16 (m, 3H), 2.06-1.93 (, 2H), 1.762-1.54 (i, 6H), 1.26-1.03 (in, 5H).

The compounds described in Table 15 were prepared using the general methods outlined above.

TABLE 15
Characterization of Compounds 382-384

| Compound | | MS | HPLC | HPLC |
|---|---|---|---|---|
| # | Structure | (M + H)⁺ | Rt | cond. |
| 382 | 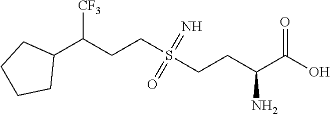 [SMILES: N=S(=O)(CCC(C1CCCC1)C(F)(F)F)CC[C@H](N)C(=O)O] | 345.0 | 1.821 | F |
| | ¹H NMR (400 MHz, D₂O) δ 3.85-3.77 (m, 1H), | | | |
| | 3.46-3.25 (m, 4H), 2.39-2.25 (m, 3H), 2.13-1.93 | | | |
| | (m, 3H), 1.87-1.70 (m, 2H), 1.67-1.40 (m, 4H), | | | |
| | 1.35-1.10 (m, 2H) | | | |
| 383 | 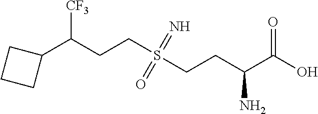 [SMILES: N=S(=O)(CCC(C1CCC1)C(F)(F)F)CC[C@H](N)C(=O)O] | 331.1 | 1.777 | C |
| | ¹H NMR (400 MHz, D₂O) δ 3.86-3.77 (m, 1H), | | | |
| | 3.42 (br d, 4H), 2.55-2.33 (m, 2H), 2.32-2.20 (m, | | | |
| | 2H), 1.99 (br d, 2H), 1.91 (br dd, 2H), 1.86-1.72 | | | |
| | (m, 3H), 1.67 (br d, 1H) | | | |
| 384 | 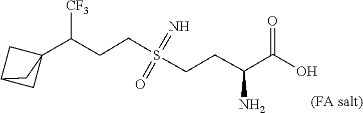 [SMILES: N=S(=O)(CCC(C(F)(F)F)C12CC(C1)C2)CC[C@H](N)C(=O)O] | 343.1 | 2.228 | B |
| | ¹H NMR (400 MHz, D₂O) δ 8.41 (s, 1H), 3.92- | | | |
| | 3.78 (m, 1H), 3.52-3.26 (m, 4H), 3.15-2.87 (m, | | | |
| | 2H), 2.71-2.23 (m, 8H), 1.10 (br d, 2H) | | | |

### Exemplary Embodiment 1aa26

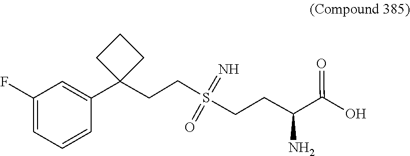 [SMILES: N=S(=O)(CC[C@H](N)C(=O)O)CCC1(c2cccc(F)c2)CCC1]

### (2S)-2-amino-4-[2-[1-(3-fluorophenyl)cyclobutyl]ethylsulfonimidoyl]butanoic acid

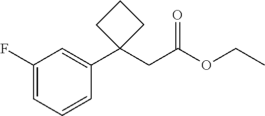 [SMILES: CCOC(=O)CC1(c2cccc(F)c2)CCC1]

A mixture of (3-fluorophenyl)boronic acid (1.50 g, 10.7 mmol, 1.5 eq), ethyl 2-cyclobutylideneacetate (1 g, 7.13 mmol, 1 eq), KOH (1.5 M, 6.18 mL, 1.3 eq), and chloro(1,5-cyclooctadiene)rhodium(I) dimer (CAS 12092-47-6, 703 mg, 1.43 mmol, 0.2 eq) in dioxane (10 mL) was degassed and purged 3 times with N₂, and then the mixture was stirred at 20° C. for 16 h under N₂ atmosphere. The reaction mixture was quenched with water (5 mL) and extracted with EtOAc (10 mL×3). The combined organic layers were washed with brine (10 mL×1), dried over Na₂SO₄, filtered, and concentrated under reduced pressure. The residue was purified by column chromatography (SiO₂, petroleum ether/EtOAc=20:1 to 10:1) to give ethyl 2-(1-(3-fluorophenyl)cyclobutyl)acetate (940 mg, 3.98 mmol, 55.7% yield) as a colorless oil. ¹H NMR (400 MHz, CDCl₃-d) δ ppm 7.29-7.20 (m, 1H), 6.94 (d, 1H), 6.90-6.82 (m, 2H), 4.14 (q, 1H), 2.77 (s, 2H), 2.47-2.33 (m, 4H), 2.16-2.02 (m, 2H), 1.95-1.79 (m, 1H), 1.07 (t, 3H).

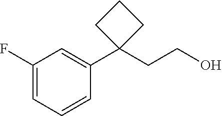 [SMILES: OCCC1(c2cccc(F)c2)CCC1]

A solution of ethyl 2-(1-(3-fluorophenyl)cyclobutyl)acetate (940 mg, 3.98 mmol, 1 eq) and LAH (150 mg, 3.98 mmol, 1 eq) in THE (20 mL) was degassed and purged 3 times with N₂, and then the mixture was stirred at 20° C. for 1 h under N₂ atmosphere. The reaction mixture was quenched with water (0.15 mL) and 15%, aq. NaOH (0.15 mL) at 0° C. and then filtered and concentrated under reduced pressure to give crude product as colorless oil. The residue was purified by column chromatography (SiO₂, petroleum ether/EtOAc=10:1 to 5:1) to give 2-[1-(3-fluorophenyl)cyclobutyl]ethanol (700 mg, 3.60 mmol, 90.58% yield) as a colorless oil. ¹H NMR (400 MHz, CDCl₃-d) δ ppm 7.32-7.23 (m, 1H), 6.94-6.82 (m, 3H), 3.72-3.56 (m, 1H), 3.45 (t, 2H), 2.44-2.30 (m, 2H), 2.25-1.96 (m, 6H), 1.92-1.64 (m, 3H).

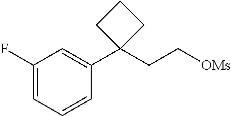 [SMILES: CS(=O)(=O)OCCC1(c2cccc(F)c2)CCC1]

To a solution of 2-[1-(3-fluorophenyl)cyclobutyl]ethanol (700 mg, 3.60 mmol, 1 eq) in DCM (15 mL) was added TEA (729 mg, 7.21 mmol, 1.00 mL, 2 eq) and methylsulfonyl methanesulfonate (753 mg, 4.32 mmol, 1.2 eq). The mixture was stirred at 20° C. for 1 h. The reaction mixture was quenched with water (10 mL) and then extracted with EtOAc (10 mL×3). The combined organic layers were washed with brine (10 mL×1), dried over Na₂SO₄, filtered, and concentrated under reduced pressure to give a residue. The residue was purified by column chromatography (SiO₂, petroleum ether/EtOAc=10:1 to 5:1) to give 2-[1-(3-fluorophenyl)cyclobutyl]ethyl methanesulfonate (740 mg, 2.72 mmol, 75.40% yield) as a yellow oil. H NMR (400 MHz, CDCl₃-d) δ 7.31-7.20 (m, 1H), 6.92-6.77 (m, 3H), 3.93 (t, 2H), 2.86 (s, 3H), 2.43-2.30 (m, 2H), 2.28-2.02 (m, 6H), 1.94-1.73 (m, 2H).

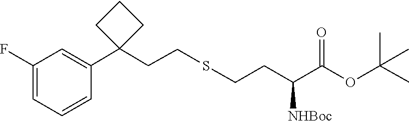 [SMILES: CC(C)(C)OC(=O)N[C@@H](CCSCCC1(c2cccc(F)c2)CCC1)C(=O)OC(C)(C)C]

A mixture of 2-[1-(3-fluorophenyl)cyclobutyl]ethyl methanesulfonate (740 mg, 2.72 mmol, 1 eq), tert-butyl (2S)-2-(tert-butoxycarbonylamino)-4-sulfanyl-butanoate (791 mg, 2.72 mmol, 1 eq), K₂CO₃ (1.13 g, 8.15 mmol, 3 eq), and KI (902 mg, 5.43 mmol, 2 eq) in DMF (20 mL) was degassed and purged 3 times with N₂ for, and then the mixture was stirred at 40° C. for 16 h under N₂ atmosphere. The reaction mixture was quenched with water (60 mL) and the mixture was extracted with EtOAc (20 mL×3). The combined organic layers were washed with brine (20 mL×1), dried over Na₂SO₄, filtered, and concentrated under reduced pressure to give a residue. The residue was purified by column chromatography (SiO₂, petroleum ether/EtOAc=10:1 to 5:1) to give tert-butyl (2S)-2-(tert-butoxycarbonylamino)-4-[2-[1-(3-fluorophenyl)cyclobutyl]ethylsulfanyl]butanoate (780 mg, 1.67 mmol, 61.38% yield) as a colorless oil. ¹H NMR (400 MHz, CDCl₃-d) δ 7.32-7.21 (m, 2H), 6.92-6.77 (m, 3H), 4.13 (q, 1H), 2.56-2.29 (m, 5H), 2.23-1.96 (m, 8H), 1.93-1.72 (m, 3H), 1.45 (d, 18H).

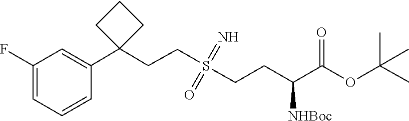 [SMILES: CC(C)(C)OC(=O)N[C@@H](CCS(=N)(=O)CCC1(c2cccc(F)c2)CCC1)C(=O)OC(C)(C)C]

A mixture of give tert-butyl (2S)-2-(tert-butoxycarbonylamino)-4-[2-[1-(3-fluorophenyl)cyclobutyl]ethylsulfanyl]butanoate (170 mg, 363 μmol, 1 eq), PhI(OAc)₂ (468 mg, 1.45 mmol, 4 eq), and ammonium carbamate (227 mg, 2.91 mmol, 8 eq) in i-PrOH (10 mL) was degassed and purged 3 times with N₂, and then the mixture was stirred at 20° C. for 16 h under N₂ atmosphere. The reaction mixture was quenched with water (10 mL) and then extracted with EtOAc (10 mL×3). The combined organic layers were washed with brine (10 mL×1), dried over Na₂SO₄, filtered, and concentrated under reduced pressure to give a residue. The residue was purified by prep-TLC (SiO₂, petroleum ether/EtOAc=1:1) to give tert-butyl (2S)-2-(tert-butoxycarbonylamino)-4-[2-[1-(3-fluorophenyl)cyclobutyl]ethylsulfonimidoyl]butanoate (150 mg, 300.81 μmol, 82.75% yield) as a colorless oil. ¹H NMR (400 MHz, CDCl₃-d) δ 7.32-7.28 (m, 1H), 6.93-6.84 (m, 2H), 6.80 (br d, 1H), 4.13 (q, 1H), 2.80-2.63 (m, 3H), 2.45-2.22 (m, 6H), 2.19-2.10 (m, 3H), 2.06 (s, 2H), 1.46 (d, 20H).

A solution of tert-butyl (2S)-2-(tert-butoxycarbonylamino)-4-[2-[1-(3-fluorophenyl)cyclobutyl]ethylsulfonimidoyl]butanoate (150 mg, 300 μmol, 1 eq) in HCl/dioxane (10 mL) was stirred at 40° C. for 16 h. The reaction mixture was and the crude residue was purified by reverse-phase HPLC (C₁₈-1 (150×30 mm, 5 μm), mobile phase: [water (NH₄HCO₃)-MeCN]; gradient: 20-50% B) to give (2S)-2-amino-4-[2-[1-(3-fluorophenyl)cyclobutyl]ethylsulfonimidoyl]butanoic acid (29 mg, 84.69 μmol, 28.15% yield). LCMS: Rt=0.620 min, (ES⁺) m/z (M+H)⁺=343.1, HPLC Conditions: C. ¹H NMR (400 MHz, CDCl₃-d) δ 7.46-7.28 (m, 1H), 7.09-6.89 (m, 3H), 3.75 (br d, 1H), 3.36-3.13 (m, 2H), 2.95-2.86 (m, 2H), 2.41-2.31 (m, 2H), 2.31-2.23 (m, 2H), 2.21-2.05 (m, 5H), 1.88-1.78 (m, 1H).

### Exemplary Embodiment 1aa27

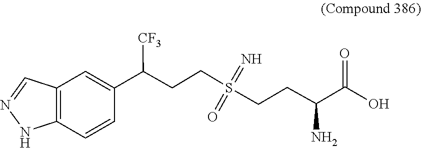 [SMILES: N=S(=O)(CCC(c1ccc2[nH]ncc2c1)C(F)(F)F)CC[C@H](N)C(=O)O]

### (2S)-2-amino-4-(4,4,4-trifluoro-3-(1H-indazol-5-yl)butylsulfonimidoyl)butanoic acid

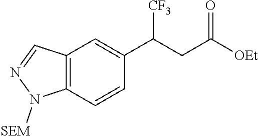 [SMILES: CCOC(=O)CC(c1ccc2c(cnn2COCC[Si](C)(C)C)c1)C(F)(F)F]

A mixture of trimethyl-[2-[[5-(4,4,5,5-tetramethyl-1,3,2-dioxaborolan-2-yl)indazol-1-yl]methoxy]ethyl]silane (1.11 g, 2.97 mmol, 1 eq), ethyl (E)-4,4,4-trifluorobut-2-enoate (500 mg, 2.97 mmol, 442 μL, 1 eq), KOH (1.5 M, 2.58 mL, 1.3 eq) and chloro(1,5-cyclooctadiene)rhodium(I) dimer (CAS 12092-47-6, 293 mg, 595 μmol, 0.2 eq) in dioxane (4.1 mL) was degassed and purged 3 times with N₂ and then the mixture was stirred at 20° C. for 16 h under N₂ atmosphere. The reaction mixture was diluted with H₂O (17 mL) and extracted with EtOAc (10 mL×3). The combined organic phase was washed with brine (4 mL), dried with anhydrous Na₂SO₄, filtered, and concentrated under reduced pressure to give a residue. The residue was purified by column chromatography (SiO₂, petroleum ether/EtOAc=100:1 to 10:1) to give ethyl 4,4,4-trifluoro-3-(1-((2-(trimethylsilyl)ethoxy)methyl)-1H-indazol-5-yl)butanoate (540 mg, 1.30 mmol, 43.59% yield) as a yellow oil. ¹H NMR (400 MHz, CDCl₃-d) δ 8.01 (s, 1H), 7.73 (s, 1H), 7.58 (d, 1H), 7.39 (d, 1H), 5.74 (s, 2H), 4.15-3.89 (m, 3H), 3.63-3.51 (m, 2H), 3.13-3.05 (m, 1H), 3.01-2.92 (m, 1H), 1.12 (t, 3H), 0.88 (br d, 2H), −0.06 (s, 9H).

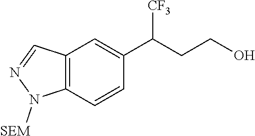 [SMILES: C[Si](C)(C)CCOCn1ncc2cc(C(CCO)C(F)(F)F)ccc21]

To a solution of ethyl 4,4,4-trifluoro-3-(1-((2-(trimethylsilyl)ethoxy)methyl)-1H-indazol-5-yl)butanoate (540 mg, 1.30 mmol, 1 eq) in THE (5 mL) was added LAH (54.1 mg, 1.43 mmol, 1.1 eq) at 0° C. The mixture was stirred at 20° C. for 2 h and then the mixture was diluted with EtOAc (5 mL), quenched with water (0.055 mL), 15% aq. NaOH (0.055 mg), and water (0.165 mL). The mixture was filtered and concentrated to give 4,4,4-trifluoro-3-(1-((2-(trimethylsilyl)ethoxy)methyl)-1H-indazol-5-yl)butan-1-ol (479 mg, 1.28 mmol, 98.66% yield) as a yellow oil. ¹H NMR (400 MHz, CDCl₃-d) δ 8.01 (s, 1H), 7.71 (s, 1H), 7.59 (d, 1H), 7.39 (d, 1H), 5.74 (s, 2H), 3.79-3.65 (m, 2H), 3.61-3.53 (m, 2H), 3.41 (dt, 1H), 2.41-2.31 (m, 1H), 2.20-2.08 (m, 1H), 0.95-0.86 (m, 2H), 0.03-−0.11 (m, 9H).

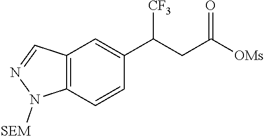 [SMILES: C[Si](C)(C)CCOCn1ncc2cc(C(CC(=O)OS(C)(=O)=O)C(F)(F)F)ccc21]

To a mixture of 4,4,4-trifluoro-3-(1-((2-(trimethylsilyl)ethoxy)methyl)-1H-indazol-5-yl)butan-1-ol (479 mg, 1.28 mmol, 1 eq) and TEA (388 mg, 3.84 mmol, 534 μL, 3 eq) in DCM (8 mL) was added methylsulfonyl methanesulfonate (334 mg, 1.92 mmol, 1.5 eq) at 0° C., and then the mixture was stirred at 20° C. for 2 h under N₂ atmosphere. The reaction mixture was quenched by addition of water (10 mL) at 20° C. and the mixture was extracted with DCM (10 mL×2). The combined organic layers were washed with brine (4 mL), dried over Na₂SO₄, filtered, and concentrated under reduced pressure to give 4,4,4-trifluoro-3-(1-((2-(trimethylsilyl)ethoxy)methyl)-1H-indazol-5-yl)butyl methanesulfonate (545 mg, 1.20 mmol, 94.15% yield) as a yellow oil. ¹H NMR (400 MHz, CDCl₃-d) δ 8.03 (s, 1H), 7.71 (s, 1H), 7.62 (d, 1H), 7.37 (d, 1H), 5.75 (s, 2H), 4.26 (td, 1H), 3.98 (dt, 1H), 3.73-3.62 (m, 1H), 3.62-3.53 (m, 2H), 2.93 (s, 3H), 2.58 (tt, 1H), 2.41-2.24 (m, 1H), 0.96-0.85 (m, 2H), −0.05 (s, 9H).

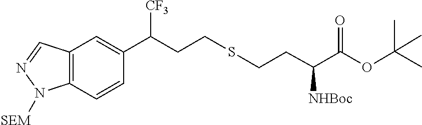 [SMILES: CC(C)(C)OC(=O)N[C@@H](CCSCCC(c1ccc2c(cnn2COCC[Si](C)(C)C)c1)C(F)(F)F)C(=O)OC(C)(C)C]

A mixture of 4,4,4-trifluoro-3-(1-((2-(trimethylsilyl)ethoxy)methyl)-1H-indazol-5-yl)butyl methanesulfonate (131 mg, 289 μmol, 1 eq), tert-butyl 2-(tert-butoxycarbonylamino)-4-sulfanyl-butanoate (101 mg, 347 μmol, 1.2 eq), K₂CO₃ (120 mg, 868 μmol, 3 eq) and KI (96.1 mg, 579 μmol, 2 eq) in DMF (2 mL) was degassed and purged 3 times with Ar, and then the mixture was stirred at 50° C. for 16 h under Ar atmosphere. The reaction mixture was diluted with water (10 mL), extracted with EtOAc (10 mL×3), and the combined organic phase was washed with brine (10 mL), dried with anhydrous Na₂SO₄, filtered, and concentrated under reduced pressure to give a residue. The residue was purified by column chromatography (SiO₂, petroleum ether/EtOAc=100:1 to 4:1) to give (2S)-tert-butyl 2-((tert-butoxycarbonyl)amino)-4-((4,4,4-trifluoro-3-(1-((2-(trimethylsilyl)ethoxy)methyl)-1H-indazol-5-yl)butyl)thio)butanoate (178 mg, 274.75 μmol, 94.92% yield) as a yellow oil. ¹H NMR (400 MHz, CDCl₃-d) δ 8.02 (d, 1H), 7.69 (s, 1H), 7.59 (d, 1H), 7.37 (d, 1H), 5.74 (s, 2H), 5.09 (br d, 1H), 4.26 (br d, 1H), 3.61-3.54 (m, 2H), 2.81-2.62 (m, 1H), 2.57-2.44 (m, 3H), 2.35-2.21 (m, 3H), 2.04-1.95 (m, 1H), 1.89-1.77 (m, 1H), 1.46-1.44 (m, 18H), 0.94-0.88 (m, 2H), −0.06 (s, 9H).

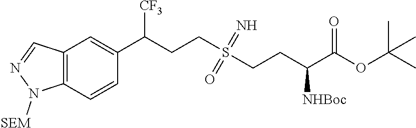 [SMILES: CC(C)(C)OC(=O)N[C@@H](CCS(=N)(=O)CCC(c1ccc2c(cnn2COCC[Si](C)(C)C)c1)C(F)(F)F)C(=O)OC(C)(C)C]

To a solution of (2S)-tert-butyl 2-((tert-butoxycarbonyl)amino)-4-((4,4,4-trifluoro-3-(1-((2-(trimethylsilyl)ethoxy)methyl)-1H-indazol-5-yl)butyl)thio)butanoate (178 mg, 275 μmol, 1 eq) in i-PrOH (4 mL) was added [acetoxy(phenyl)-λ³-iodanyl]acetate (354 mg, 1.10 mmol, 4 eq) and ammonium carbamate (172 mg, 2.20 mmol, 8 eq). The mixture was stirred at 25° C. for 16 h and the reaction mixture was concentrated under reduced pressure to give a residue. The residue was diluted with water (5 mL) and extracted with EtOAc (5 mL×2). The combined organic layers were washed with brine (5 mL), dried over Na₂SO₄, filtered, and concentrated under reduced pressure. The residue was purified by column chromatography (SiO₂, petroleum ether/EtOAc=4:1 to 1:1) to give (2S)-tert-butyl 2-((tert-butoxycarbonyl)amino)-4-(4,4,4-trifluoro-3-(1-((2-(trimethylsilyl)ethoxy)methyl)-1H-indazol-5-yl)butylsulfonimidoyl)butanoate (100 mg, 147.30 μmol, 53.61% yield) as a yellow oil. ¹H NMR (400 MHz, CDCl₃-d) δ 8.03 (d, 1H), 7.71 (s, 1H), 7.62 (d, 1H), 7.36 (d, 1H), 5.75 (s, 2H), 5.19 (br s, 1H), 4.24 (br d, 1H), 3.72-3.63 (m, 1H), 3.60-3.55 (m, 2H), 3.19-3.04 (m, 1H), 3.03-2.76 (m, 3H), 2.71-2.43 (m, 3H), 2.39-2.30 (m, 1H), 2.12-2.03 (m, 1H), 1.47-1.43 (m, 18H), 0.93 (d, 2H), −0.05 (s, 9H).

A solution of (2S)-tert-butyl 2-((tert-butoxycarbonyl)amino)-4-(4,4,4-trifluoro-3-(1-((2-(trimethylsilyl)ethoxy)methyl)-1H-indazol-5-yl)butylsulfonimidoyl)butanoate (100 mg, 147.30 μmol, 1 eq) in HCl/dioxane (9 mL) and water (0.9 mL) was stirred at 20° C. for 12 h. The reaction mixture was concentrated and the residue was purified by prep-HPLC (Waters Xbridge Prep OBD C18 (150×40 mm, 10 μm); mobile phase: [water (NH₄HCO₃)-MeCN]; gradient: 15-55% B) to give (2S)-2-amino-4-(4,4,4-trifluoro-3-(1H-indazol-5-yl)butylsulfonimidoyl)butanoic acid (11.6 mg, 29.18 μmol, 19.81% yield) as a white solid. LCMS: Rt=1.836 min, (ES⁺) m/z (M+H)⁺=393.2, HPLC Conditions: B. ¹H NMR (400 MHz, D₂O) δ 8.17 (s, 1H), 7.91 (s, 1H), 7.70 (d, 1H), 7.50 (d, 1H), 3.94-3.73 (m, 2H), 3.44-3.21 (m, 3H), 3.09-2.91 (m, 1H), 2.69-2.43 (m, 2H), 2.30-2.17 (m, 2H).

### Exemplary Embodiment 1aa28

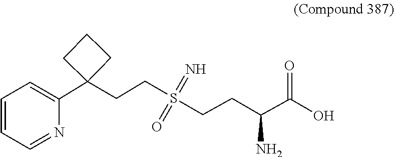 [SMILES: N=S(=O)(CC[C@H](N)C(=O)O)CCC1(c2ccccn2)CCC1]

### (2S)-2-amino-4-(2-(1-(pyridin-2-yl)cyclobutyl)ethylsulfonimidoyl)butanoic acid

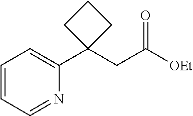 [SMILES: CCOC(=O)CC1(c2ccccn2)CCC1]

A mixture of ethyl 2-cyclobutylideneacetate (532 mg, 3.80 mmol, 1.2 eq), 2-bromopyridine (500 mg, 3.16 mmol, 301.20 μL, 1 eq), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (35.5 mg, 31.7 μmol, 0.01 eq) and diethyl 2,6-dimethyl-1,4-dihydropyridine-3,5-dicarboxylate (1.04 g, 4.11 mmol, 1.3 eq) in DMSO (15 mL) and H₂O (5 mL) was stirred at 20° C. under blue LED irradiation (λ=440 nm) for 16 h. The mixture was adjusted to pH=8 with sat. aq. NaHCO₃ and then extracted with EtOAc (20 mL×2). The combined organic phases were washed with brine (10 mL), dried over Na₂SO₄, filtered, and concentrated. The residue was purified by chromatography (SiO₂, petroleum ether/EtOAc=5:1) to give ethyl 2-(1-(pyridin-2-yl)cyclobutyl)acetate (0.14 g, 638 μmol, 10.09% yield) as yellow oil. ¹H NMR (400 MHz, CDCl₃-d) δ 8.56 (dd, 1H), 7.63 (dt, 1H), 7.30 (d, 1H), 7.09 (dd, 1H), 3.96 (q, 2H), 2.97 (s, 2H), 2.60-2.50 (m, 2H), 2.39-2.30 (m, 2H), 2.20-2.03 (m, 1H), 1.90 (ttd1H), 1.08 (t, 3H).

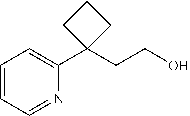 [SMILES: OCCC1(c2ccccn2)CCC1]

To a solution of ethyl 2-[1-(2-pyridyl)cyclobutyl]acetate (0.14 g, 638 μmol, 1 eq) in THE (5 mL) was added LAH (24.2 mg, 638 μmol, 1 eq) at 0° C., and the mixture was stirred at 0-15° C. for 1 h. The mixture was quenched with water (10 mL) and extracted with EtOAc (10 mL×3). The combined organic phases were washed with brine (10 mL), dried over Na₂SO₄, filtered, and concentrated to give 2-(1-(pyridin-2-yl)cyclobutyl)ethanol (100 mg, 564 μmol, 88.37% yield) as yellow oil. ¹H NMR (400 MHz, CDCl₃-d) δ 8.63-8.39 (m, 1H), 7.68 (dt, 1H), 7.25 (s, 1H), 7.13 (ddd, 1H), 3.51 (t, 2H), 2.57-2.44 (m, 2H), 2.35-2.23 (m, 2H), 2.19-2.14 (m, 2H), 2.14-2.04 (m, 1H), 1.90-1.79 (m, 1H).

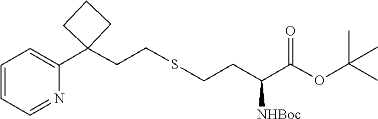 [SMILES: CC(C)(C)OC(=O)N[C@@H](CCSCCC1(c2ccccn2)CCC1)C(=O)OC(C)(C)C]

A mixture of 2-(1-(pyridin-2-yl)cyclobutyl)ethanol (100 mg, 564 μmol, 1 eq), tert-butyl (tert-butoxycarbonyl)-L-homocysteinate (329 mg, 1.13 mmol, 2 eq) and 2-(tributyl-λ⁵-phosphanylidene)acetonitrile (272 mg, 1.13 mmol, 2 eq) in toluene (5 mL) was stirred at 100° C. for 8 h. The mixture was quenched with water (10 mL) and extracted with EtOAc (10 mL×2). The combined organic phases were washed with brine (10 mL), dried over Na₂SO₄, filtered, and concentrated. The residue was purified by prep-TLC (petroleum ether/EtOAc=3:1) to give (S)-tert-butyl 2-((tert-butoxycarbonyl)amino)-4-((2-(1-(pyridin-2-yl)cyclobutyl)ethyl)thio)butanoate (100 mg, 222 μmol, 39.33% yield) as yellow oil. ¹H NMR (400 MHz, CDCl₃-d) δ 8.57 (br d, 1H), 7.64 (br t, 1H), 7.21-7.04 (m, 2H), 5.13-4.96 (m, 1H), 4.29-4.15 (m, 1H), 2.56-2.43 (m, 4H), 2.31-2.05 (m, 7H), 2.01-1.92 (m, 1H), 1.88-1.70 (m, 2H), 1.45 (d, 18H).

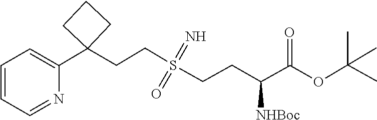 [SMILES: CC(C)(C)OC(=O)N[C@@H](CCS(=N)(=O)CCC1(c2ccccn2)CCC1)C(=O)OC(C)(C)C]

A mixture of (S)-tert-butyl 2-((tert-butoxycarbonyl)amino)-4-((2-(1-(pyridin-2-yl)cyclobutyl)ethyl)thio)butanoate (100 mg, 222 μmol, 1 eq), PhI(OAc)₂ (214 mg, 666 μmol, 3 eq) and ammonium carbamate (104 mg, 1.33 mmol, 6 eq) in i-PrOH (5 mL) was stirred at 20° C. for 12 h. The mixture was quenched with water (10 mL) and extracted with EtOAc (10 mL×2), and the combined organic phases were washed with brine (10 mL), dried over Na₂SO₄, filtered, and concentrated. The residue was purified by prep-TLC (EtOAc/methanol=20:1) to give (2S)-tert-butyl 2-((tert-butoxycarbonyl)amino)-4-(2-(1-(pyridin-2-yl)cyclobutyl)ethylsulfonimidoyl)butanoate (40 mg, 83.05 μmol, 37.42% yield) as yellow oil.

A solution of (2S)-tert-butyl 2-((tert-butoxycarbonyl)amino)-4-(2-(1-(pyridin-2-yl)cyclobutyl)ethylsulfonimidoyl)butanoate (70.0 mg, 145 μmol, 1 eq) in HCl/dioxane (20 mL, 6M) was stirred at 25° C. for 3 h. The mixture was concentrated and the residue was purified by prep-HPLC (column: Phenomenex C18 75×30 mm×3 μm; mobile phase: [water (NH₄HCO₃)-MeCN]; B %: 5-35%, 10 min) to give (2S)-2-amino-4-(2-(1-(pyridin-2-yl)cyclobutyl)ethylsulfonimidoyl)butanoic acid (20 mg, 60.6 μmol, 41.67% yield) as white solid. LCMS: Rt=0.302 min, (ES⁺) m/z (M+H)⁺=326.1, HPLC Conditions: C. ¹H NMR (400 MHz, D₂O) δ 8.42 (br d, 1H), 7.83 (br t, 1H), 7.34 (d, 1H), 7.28 (dd, 1H), 3.84-3.74 (m, 1H), 3.39-3.13 (m, 2H), 2.99-2.84 (m, 2H), 2.52-2.40 (m, 2H), 2.39-2.29 (m, 2H), 2.27-2.08 (m, 5H), 1.90-1.76 (m, 1H).

### Exemplary Embodiment 1aa29 (Isomer 1) & 1aa29 (Isomer 2)

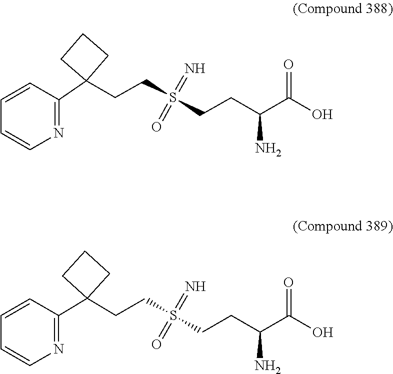 [SMILES: N=[S@@](=O)(CC[C@H](N)C(=O)O)CCC1(c2ccccn2)CCC1 ; N=[S@](=O)(CC[C@H](N)C(=O)O)CCC1(c2ccccn2)CCC1]

### (S)-2-amino-4-((S)-2-(1-(pyridin-2-yl)cyclobutyl)ethylsulfonimidoyl)butanoic acid (Isomer 1) & (S)-2-amino-4-((R)-2-(1-(pyridin-2-yl)cyclobutyl)ethylsulfonimidoyl)butanoic acid (Isomer 2)

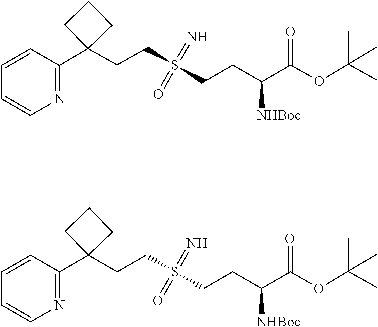 [SMILES: CC(C)(C)OC(=O)N[C@@H](CC[S@@](=N)(=O)CCC1(c2ccccn2)CCC1)C(=O)OC(C)(C)C ; CC(C)(C)OC(=O)N[C@@H](CC[S@](=N)(=O)CCC1(c2ccccn2)CCC1)C(=O)OC(C)(C)C]

A mixture of (S)-tert-butyl 2-((tert-butoxycarbonyl)amino)-4-((2-(1-(pyridin-2-yl)cyclobutyl)ethyl)thio)butanoate (349 mg, 774 μmol, 1 eq), ammonium carbamate (484 mg, 6.20 mmol, 8 eq) and PhI(OAc)₂ (998 mg, 3.10 mmol, 4 eq) in i-PrOH (6 mL) was stirred at 25° C. for 12 h under N₂ atmosphere. The reaction mixture was concentrated under reduced pressure and the resultant residue was diluted with water (60 mL) and extracted with EtOAc (60 mL×2). The combined organic layers were washed with brine (100 mL), dried over Na₂SO₄, filtered, and concentrated under reduced pressure. The residue was purified by column chromatography (SiO₂, petroleum ether/EtOAc=12:88 to 0:1) to give (2S)-tert-butyl 2-((tert-butoxycarbonyl)amino)-4-(2-(1-(pyridin-2-yl)cyclobutyl)ethylsulfonimidoyl)butanoate (0.19 g, yield: 50.9%), which was then separated by SFC (column: DAICEL CHIRALCEL OX (250×30 mm, 10 μm); mobile phase: [CO₂-MeOH (0.1% NH₃H₂O)]; B: 30%, isocratic elution mode). The separated compounds were tentatively assigned stereochemistry and notated by their peak elution order. tert-Butyl (S)-2-((tert-butoxycarbonyl)amino)-4-((S)-2-(1-(pyridin-2-yl)cyclobutyl)ethylsulfonimidoyl)butanoate (95 mg, 197 μmol, 25.5% yield) was obtained as a yellow oil and tert-butyl (S)-2-((tert-butoxycarbonyl)amino)-4-((R)-2-(1-(pyridin-2-yl)cyclobutyl)ethylsulfonimidoyl)butanoate (85 mg, 176 μmol, 22.8% yield) was obtained as a yellow oil.

A solution of tert-butyl (S)-2-((tert-butoxycarbonyl)amino)-4-((S)-2-(1-(pyridin-2-yl)cyclobutyl)ethylsulfonimidoyl)butanoate (95 mg, 201 μmol, 1 eq) in HCl/dioxane (10 mL, 6 M) was stirred at 30° C. for 4 h. The reaction mixture was concentrated under reduced pressure and the residue was purified by prep-HPLC (column: Phenomenex Gemini NX C18 (150×10 mm, 5 μm); mobile phase: [H₂O (0.1% TFA)-MeCN]; gradient: 1-20% B over 10.0 min) to give (S)-2-amino-4-((S)-2-(1-(pyridin-2-yl)cyclobutyl)ethylsulfonimidoyl)butanoic acid (Isomer 1, 28.47 mg, 87.49 μmol, 43.44% yield) as a white solid. LCMS: Rt=1.914 min, (ES⁺) m/z (M+H)⁺=326.1, HPLC Conditions: I. ¹H NMR (400 MHz, MeOD-d₄) δ 8.50 (br d, 1H), 7.86-7.77 (m, 1H), 7.36 (br d, 1H), 7.26 (dd, 1H), 3.65 (br t, 1H), 3.38-3.32 (m, 1H), 3.26-3.15 (m, 1H), 2.91-2.77 (m, 2H), 2.58-2.48 (m, 2H), 2.46-2.36 (m, 2H), 2.33-2.14 (m, 5H), 1.96-1.83 (m, 1H).

A solution of and tert-butyl (S)-2-((tert-butoxycarbonyl)amino)-4-((R)-2-(1-(pyridin-2-yl)cyclobutyl)ethylsulfonimidoyl)butanoate (85 mg, 176 μmol, 1 eq) in HCl/dioxane (10 mL, 6 M) was stirred at 30° C. for 4 h. The reaction mixture was concentrated under reduced pressure and the residue was purified by prep-HPLC (column: Phenomenex Gemini NX C18 (150×10 mm, 5 μm); mobile phase: [H₂O (0.1% TFA)-MeCN]; gradient: 10% B over 10.0 min) to give (S)-2-amino-4-((R)-2-(1-(pyridin-2-yl)cyclobutyl)ethylsulfonimidoyl)butanoic acid (Isomer 2, 30.6 mg, 94 μmol, 53.2% yield) as a white solid. LCMS: Rt=1.866 min, (ES⁺) m/z (M+H)⁺=326.1, HPLC Conditions: I. ¹H NMR (400 MHz, MeOD-d₄) δ 8.53-8.47 (m, 1H), 7.81 (dt, 1H), 7.35 (d, 1H), 7.26 (dt, 1H), 3.67 (t, 1H), 3.29-3.15 (m, 2H), 2.90-2.77 (m, 2H), 2.57-2.48 (m, 2H), 2.46-2.37 (m, 2H), 2.31-2.15 (m, 5H), 1.96-1.82 (m, 1H).

### Exemplary Embodiment 1aa30

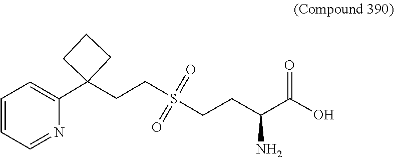 [SMILES: N[C@@H](CCS(=O)(=O)CCC1(c2ccccn2)CCC1)C(=O)O]

### (2S)-2-amino-4-(3-(4-(2,4-dichlorophenyl)thiophen-2-yl)-4,4,4-trifluoro-3-hydroxybutylsulfonimidoyl)butanoic acid

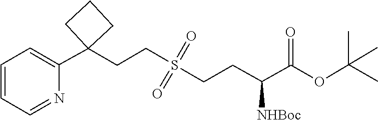 [SMILES: CC(C)(C)OC(=O)N[C@@H](CCS(=O)(=O)CCC1(c2ccccn2)CCC1)C(=O)OC(C)(C)C]

To a solution of (S)-tert-butyl 2-((tert-butoxycarbonyl)amino)-4-((2-(1-(pyridin-2-yl)cyclobutyl)ethyl)thio)butanoate (120 mg, 266 μmol, 1 eq) in DCM (3 mL) was added m-CPBA (27.0 mg, 133 μmol, 85% purity, 0.5 eq) at 0° C. The resulting mixture was stirred at 15° C. for 1 h. The reaction mixture was poured into saturated aqueous Na₂SO₃ (20 mL) and extracted with DCM (20 mL×2). The combined organic layers were washed with brine (40 mL), dried over Na₂SO₄, filtered, and concentrated under reduced pressure. The residue was purified by prep-TLC (SiO₂, petroleum ether/EtOAc=1:1) to give tert-butyl (S)-2-((tert-butoxycarbonyl)amino)-4-((2-(1-(pyridin-2-yl)cyclobutyl)ethyl)sulfonyl)butanoate (30 mg, 62.2 μmol, 23.3% yield) as a yellow oil. ¹H NMR (400 MHz, CDCl₃-d) δ 8.56 (d, 1H), 7.67 (br t, 1H), 7.21 (d, 1H), 7.14 (dd, 1H), 5.23-5.08 (m, 1H), 4.22 (br dd, 1H), 3.09-2.97 (m, 1H), 2.97-2.85 (m, 1H), 2.79-2.69 (m, 2H), 2.58-2.47 (m, 2H), 2.46-2.38 (m, 2H), 2.36-2.25 (m, 1H), 2.23-2.12 (m, 3H), 2.06-1.85 (m, 2H), 1.47 (s, 9H), 1.45 (s, 9H).

A mixture of tert-butyl (S)-2-((tert-butoxycarbonyl)amino)-4-((2-(1-(pyridin-2-yl)cyclobutyl)ethyl)sulfonyl)butanoate (30 mg, 62.2 μmol, 1 eq) in HCl/dioxane (20 mL) (6 M) was stirred at 30° C. for 12 h. The reaction mixture was concentrated under reduced pressure and the residue was purified by prep-HPLC (column: Phenomenex Gemini-NX C18 (75×30 mm, 3 μm); mobile phase: [H₂O (10 mM NH₄HCO₃)-MeCN]; gradient: 10-40% B over 9.0 min) to give (S)-2-amino-4-((2-(1-(pyridin-2-yl)cyclobutyl)ethyl)sulfonyl)butanoic (11.9 mg, 36.5 μmol, 58.8% yield) as a yellow oil. LC-MS: Rt=1.367 min, (ES⁺) m/z (M+H)⁺=327.1, HPLC Conditions: New. ¹H NMR (400 MHz, D₂O) δ 8.39 (dd, 1H), 7.79 (dt, 1H), 7.31 (d, 1H), 7.25 (ddd, 1H), 3.72 (t, 1H), 3.31-3.12 (m, 2H), 2.95-2.87 (m, 2H), 2.49-2.38 (m, 2H), 2.37-2.28 (m, 2H), 2.19-2.07 (m, 5H), 1.87-1.75 (m, 1H).

### Exemplary Embodiment 1aa31

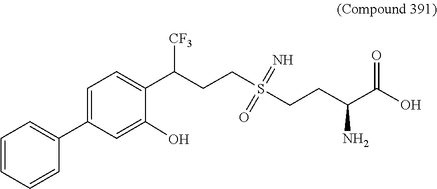 [SMILES: N=S(=O)(CCC(c1ccc(-c2ccccc2)cc1O)C(F)(F)F)CC[C@H](N)C(=O)O]

### (2S)-2-amino-4-(4,4,4-trifluoro-3-(3-hydroxy-[1,1′-biphenyl]-4-yl)butylsulfonimidoyl)butanoic acid

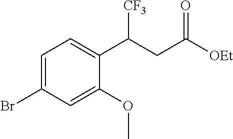 [SMILES: CCOC(=O)CC(c1ccc(Br)cc1OC)C(F)(F)F]

To a solution of (4-bromo-2-methoxyphenyl)boronic acid (570 mg, 2.47 mmol, 1 eq), ethyl (Z)-4,4,4-trifluorobut-2-enoate (415 mg, 2.47 mmol, 1 eq) in dioxane (3 mL) was added [Rh(COD)CI]₂ (122 mg, 247 μmol, 0.1 eq) and KOH (1.5 M, 2.14 mL, 1.3 eq) in water (2.14 mL). The mixture was stirred at 20° C. for 12 h. The reaction mixture was diluted with water (10 mL) and extracted with EtOAc (30 mL×3). The combined organic layers were dried over Na₂SO₄, filtered, and concentrated under reduced pressure. The residue was purified by column chromatography (SiO₂, petroleum ether/EtOAc=100:0 to 89:11) to give ethyl 3-(4-bromo-2-methoxyphenyl)-4,4,4-trifluorobutanoate (580 mg, 1.50 mmol, 60.7% yield) as a yellow oil. ¹H NMR (400 MHz, CDCl₃-d) δ 7.19-7.01 (m, 3H), 4.67-4.49 (m, 1H), 4.20-3.99 (m, 2H), 3.94-3.83 (m, 3H), 3.08-2.95 (m, 1H), 2.92-2.79 (m, 1H), 1.26-1.14 (m, 3H).

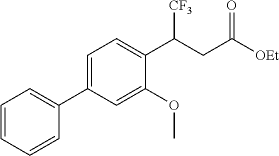 [SMILES: CCOC(=O)CC(c1ccc(-c2ccccc2)cc1OC)C(F)(F)F]

A mixture of compound phenylboronic acid (309 mg, 2.53 mmol, 3 eq), ethyl 3-(4-bromo-2-methoxyphenyl)-4,4,4-trifluorobutanoate (300 mg, 845 μmol, 1 eq), K₂CO₃ (350 mg, 2.53 mmol, 3 eq) and Pd(dppf)Cl₂·CH₂Cl₂ (69.0 mg, 84.5 μmol, 0.1 eq) in dioxane (10 mL), H₂O (2 mL) was stirred at 100° C. for 12 h under N₂ atmosphere. The combined reaction mixture was poured into water (10 mL) and extracted with EtOAc (30 mL×3). The combined organic phase was concentrated in vacuo to give a residue. The residue was purified by column chromatography (SiO₂, petroleum ether/EtOAc=100:0 to 90:10) to give compound ethyl 4,4,4-trifluoro-3-(3-methoxy-[1,1′-biphenyl]-4-yl)butanoate (260 mg, 738 μmol, 87.4% yield) as a yellow oil. ¹H NMR (400 MHz, CDCl₃-d) δ 7.54-7.48 (m, 2H), 7.37 (t, 2H), 7.32-7.24 (m, 2H), 7.11 (dd1H), 7.05-6.99 (m, 1H), 4.58 (dt, 1H), 4.18-3.93 (m, 2H), 3.90-3.77 (m, 3H), 3.04-2.90 (m, 1H), 2.89-2.75 (m, 1H), 1.23-0.99 (m, 3H).

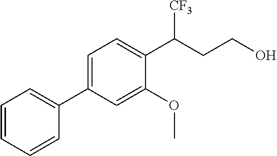 [SMILES: COc1cc(-c2ccccc2)ccc1C(CCO)C(F)(F)F]

To a solution of ethyl 4,4,4-trifluoro-3-(3-methoxy-[1,1′-biphenyl]-4-yl)butanoate (460 mg, 1.31 mmol, 1 eq) in THF (10 mL) was added LiAlH₄ (74.3 mg, 1.96 mmol, 1.5 eq) at 0° C. The mixture was stirred at 20° C. for 2 h. The reaction mixture was diluted with EtOAc (20 mL), cooled to 0° C., and quenched by addition water (80 mg), aqueous NaOH (80 mg), water (240 mg) in that order. The mixture was filtered, and the filtrate was dried over Na₂SO₄, filtered and concentrated under reduced pressure to give 4,4,4-trifluoro-3-(3-methoxy-[1,1′-biphenyl]-4-yl)butan-1-ol (430 mg, crude) as a yellow oil. ¹H NMR (400 MHz, CDCl₃-d) δ 7.51 (br d, 2H), 7.43-7.26 (m, 4H), 7.15 (dd, 1H), 7.08-7.00 (m, 1H), 4.19 (dt, 1H), 3.90-3.81 (m, 3H), 3.65-3.50 (m, 1H), 3.43-3.30 (m, 1H), 2.34-2.17 (m, 1H), 1.54 (br t, 1H).

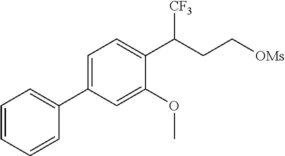 [SMILES: COc1cc(-c2ccccc2)ccc1C(CCOS(C)(=O)=O)C(F)(F)F]

To a solution of 4,4,4-trifluoro-3-(3-methoxy-[1,1′-biphenyl]-4-yl)butan-1-ol (400 mg, 1.29 mmol, 1 eq) in DCM (10 mL) was added TEA (391 mg, 3.87 mmol, 538 μL, 3 eq) and methylsulfonyl methanesulfonate (449 mg, 2.58 mmol, 2 eq) at 0° C. The mixture was stirred at 20° C. for 2 h. The reaction mixture was quenched by addition water (8 mL) at 0° C. and extracted with DCM (30 mL×3). The combined organic layers were dried over Na₂SO₄, filtered, and concentrated under reduced pressure. The residue was purified by column chromatography (SiO₂, petroleum ether/EtOAc=100:0 to 85:15) to give 4,4,4-trifluoro-3-(3-methoxy-[1,1′-biphenyl]-4-yl)butyl methanesulfonate (437 mg, 1.13 mmol, 87.3% yield) as a yellow oil. ¹H NMR (400 MHz, CDCl₃-d) δ 7.55-7.47 (m, 2H), 7.42-7.35 (m, 2H), 7.33-7.26 (m, 2H), 7.14 (dd, 1H), 7.07-7.00 (m, 1H), 4.31-4.09 (m, 2H), 3.97 (dt, 1H), 3.90-3.80 (m, 3H), 2.85 (s, 3H), 2.51-2.35 (m, 1H), 2.20 (tdd, 1H).

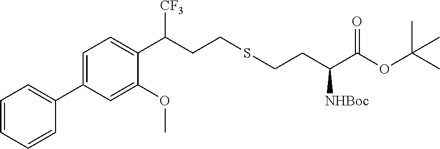 [SMILES: COc1cc(-c2ccccc2)ccc1C(CCSCC[C@H](NC(=O)OC(C)(C)C)C(=O)OC(C)(C)C)C(F)(F)F]

To a solution of compound 4,4,4-trifluoro-3-(3-methoxy-[1,1′-biphenyl]-4-yl)butyl methanesulfonate (420 mg, 1.08 mmol, 1 eq), tert-butyl (tert-butoxycarbonyl)-L-homocysteinate (945 mg, 3.24 mmol, 3 eq) in DMF (15 mL) was added KI (359 mg, 2.16 mmol, 2 eq) and K₂CO₃ (448 mg, 3.24 mmol, 3 eq) in the glove box. The mixture was stirred at 70° C. for 12 h. The reaction mixture was diluted with water (12 mL) and extracted with EtOAc (40 mL×3). The combined organic layers were dried over Na₂SO₄, filtered, and concentrated under reduced pressure. The residue was purified by column chromatography (SiO₂, petroleum ether/EtOAc=100:0 to 85:15) to give tert-butyl N-(tert-butoxycarbonyl)-S-(4,4,4-trifluoro-3-(3-methoxy-[1,1′-biphenyl]-4-yl)butyl)-L-homocysteinate (710 mg, crude) as a yellow oil. ¹H NMR (400 MHz, CDCl₃-d) δ 7.64-7.54 (m, 2H), 7.46 (t, 2H), 7.40-7.34 (m, 2H), 7.21 (dd, 1H), 7.12 (s, 1H), 5.08 (br d, 2H), 4.26-4.19 (m, 1H), 3.92 (s, 3H), 2.61-2.44 (m, 4H), 2.41-2.21 (m, 2H), 1.95-1.78 (m, 2H), 1.46-1.43 (m, 18H).

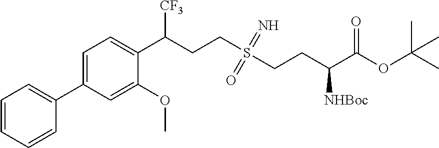 [SMILES: COc1cc(-c2ccccc2)ccc1C(CCS(=N)(=O)CC[C@H](NC(=O)OC(C)(C)C)C(=O)OC(C)(C)C)C(F)(F)F]

A mixture of tert-butyl N-(tert-butoxycarbonyl)-S-(4,4,4-trifluoro-3-(3-methoxy-[1,1′-biphenyl]-4-yl)butyl)-L-homocysteinate (610 mg, 1.05 mmol, 1 eq), PhI(OAc)₂ (1.01 g, 3.14 mmol, 3 eq), ammonium carbamate (408 mg, 5.23 mmol, 5 eq) in i-PrOH (20 mL) was stirred at 20° C. for 48 h. The reaction mixture was concentrated under reduced pressure to give a residue. The residue was diluted with water (12 mL) and extracted with DCM (40 mL×3). The combined organic layers were concentrated under reduced pressure. The residue was purified by column chromatography (SiO₂, petroleum ether/EtOAc=100:0 to 64:36) to give compound tert-butyl (2S)-2-((tert-butoxycarbonyl)amino)-4-(4,4,4-trifluoro-3-(3-methoxy-[1,1′-biphenyl]-4-yl)butylsulfonimidoyl)butanoate (385 mg, 626 μmol, 59.9% yield) as a white solid. ¹H NMR (400 MHz, CDCl₃-d) δ 7.51 (d, 2H), 7.43-7.35 (m, 2H), 7.32 (d, 2H), 7.15 (td, 1H), 7.05 (s, 1H), 5.18-5.07 (m, 1H), 4.22-4.05 (m, 2H), 3.84 (s, 3H), 3.03 (br dd, 1H), 2.99-2.87 (m, 2H), 2.84-2.74 (m, 1H), 2.57-2.42 (m, 1H), 2.29 (dq, 2H), 2.06-1.96 (m, 1H), 1.40-1.35 (m, 18H).

To a solution of (2S)-2-((tert-butoxycarbonyl)amino)-4-(4,4,4-trifluoro-3-(3-methoxy-[1,1′-biphenyl]-4-yl)butylsulfonimidoyl)butanoate (100 mg, 163 μmol, 1 eq) in DCM (5 mL) was added BBr₃ (204 mg, 813 mol, 78.4 μL, 5 eq) at 0° C. The mixture was stirred at 20° C. for 2 h. The reaction mixture was adjusted to pH=7 with saturated aqueous NaHCO₃ at 0° C., and the organic phase was separated and concentrated. The crude product was purified by prep-HPLC (column: Phenomenex Luna C18 (75×30 mm, 3 μm); mobile phase: [water (FA)-MeCN]; gradient: 1-50% B, 8 min) to give (2S′)-2-amino-4-(4,4,4-trifluoro-3-(3-hydroxy-[1,1′-biphenyl]-4-yl)butylsulfonimidoyl)butanoic acid (38.05 mg, 76.5 mol, 47.000 yield, FA) as a white solid. LCMS: Rt=2.008 min (ES⁺) m/z (M+H)⁺=445.2, HPLC Conditions: C. ¹H NMR (400 MHz, MeOD) δ 7.61 (br d, 2H), 7.49-7.39 (2, 3H), 7.38-7.32 (m, 1H), 7.22-7.11 (m, 2H), 4.32-4.17 (m, 1H), 3.77-13.64 (m, 1H), 3.47-3.36 (, 1H), 3.31-3.24 (m, 1H), 3.23-3.13 (m, 1H), 3.02-2.87 (m, 1H), 2.64-2.41 (m, 2H), 2.39-2.22 (n, 2H).

The compounds described in Table 16 were prepared using the general methods outlined above.

TABLE 16
Characterization of Compounds 392-403

| Compound | | MS | HPLC | HPLC |
|---|---|---|---|---|
| # | Structure | (M + H)⁺ | Rt | cond. |
| 392 | 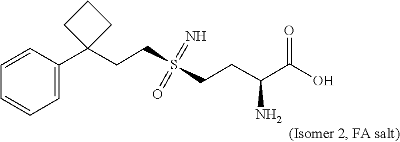 [SMILES: N=[S@@](=O)(CC[C@H](N)C(=O)O)CCC1(c2ccccc2)CCC1] | 325.1 | 1.88 | C |
| | ¹H NMR (400 MHz, D₂O) δ 7.43-7.34 (m, 2H), | | | |
| | 7.32-7.19 (m, 3H), 3.77 (t, 1H), 3.37-3.15 (m, | | | |
| | 2H), 2.98-2.86 (m, 2H), 2.43-2.32 (m, 2H), 2.32- | | | |
| | 2.24 (m, 2H), 2.22-2.06 (m, 5H), 1.88-1.78 (m, | | | |
| | 1H) | | | |
| 393 | 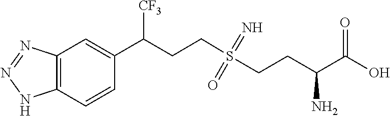 [SMILES: N=S(=O)(CCC(c1ccc2[nH]nnc2c1)C(F)(F)F)CC[C@H](N)C(=O)O] | 394.1 | 1.47 | C |
| | ¹H NMR (400 MHz, D₂O) δ 7.95-7.81 (m, 2H), | | | |
| | 7.44 (br d, 1H), 3.93-3.80 (m, 1H), 3.72 (br d, | | | |
| | 1H), 3.37-3.27 (m, 1H), 3.25-3.13 (m, 2H), 2.96- | | | |
| | 2.85 (m, 1H), 2.63-2.38 (m, 2H), 2.17 (br s, 2H) | | | |
| 394 | 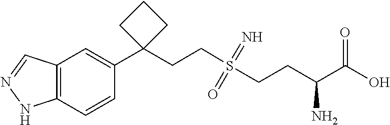 [SMILES: N=S(=O)(CC[C@H](N)C(=O)O)CCC1(c2ccc3[nH]ncc3c2)CCC1] | 365.2 | 1.89 | B |
| | ¹H NMR (400 MHz, D₂O) δ 8.04 (s, 1H), 7.63- | | | |
| | 7.54 (m, 2H), 7.27 (d, 1H), 3.73-3.65 (m, 1H), | | | |
| | 3.31-3.07 (m, 2H), 2.91-2.82 (m, 2H), 2.44-2.33 | | | |
| | (m, 2H), 2.31-2.24 (m, 2H), 2.21-2.07 (m, 5H), | | | |
| | 1.86-1.76 (m, 1H) | | | |
| 395 | 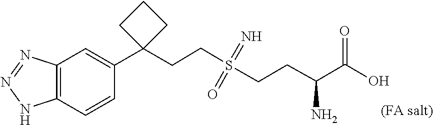 [SMILES: N=S(=O)(CC[C@H](N)C(=O)O)CCC1(c2ccc3[nH]nnc3c2)CCC1] | 366.1 | 1.58 | C |
| | ¹H NMR (400 MHz, D₂O) δ 7.86 (d, 1H), 7.63 (s, | | | |
| | 1H), 7.32 (dd, 1H), 3.76 (br d, 1H), 3.36-3.13 (m, | | | |
| | 2H), 2.96-2.83 (m, 2H), 2.48-2.39 (m, 2H), 2.39- | | | |
| | 2.31 (m, 2H), 2.28-2.10 (m, 5H), 1.91-1.80 (m, | | | |
| | 1H) | | | |
| 396 | 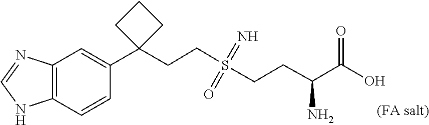 [SMILES: N=S(=O)(CC[C@H](N)C(=O)O)CCC1(c2ccc3[nH]cnc3c2)CCC1] | 365.1 | 1.26 | C |
| | ¹H NMR (400 MHz, D₂O) δ 8.80 (s, 1H), 7.74 (d, | | | |
| | 1H), 7.57 (s, 1H), 7.35 (dd, 1H), 3.81-3.71 (m, | | | |
| | 1H), 3.32-3.16 (m, 2H), 2.96-2.87 (m, 2H), 2.48- | | | |
| | 2.41 (m, 2H), 2.38-2.31 (m, 2H), 2.28-2.22 (m, | | | |
| | 2H), 2.21-2.13 (m, 3H), 1.92-1.82 (m, 1H) | | | |
| 397 | 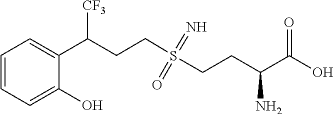 [SMILES: N=S(=O)(CCC(c1ccccc1O)C(F)(F)F)CC[C@H](N)C(=O)O] | 369.1 | 1.56 | C |
| | ¹H NMR (400 MHz, D₂O) δ 7.42 (br d, 1H), 7.30 | | | |
| | (t, 1H), 7.07-6.95 (m, 2H), 4.19 (td, 1H), 3.81 (q, | | | |
| | 1H), 3.46-3.20 (m, 3H), 3.08-2.95 (m, 1H), 2.60- | | | |
| | 2.48 (m, 1H), 2.47-2.34 (m, 1H), 2.33-2.19 (m, | | | |
| | 2H) | | | |
| 398 | 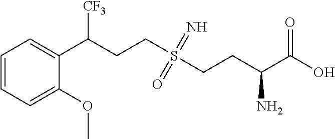 [SMILES: COc1ccccc1C(CCS(=N)(=O)CC[C@H](N)C(=O)O)C(F)(F)F] | 383.2 | 1.82 | C |
| | ¹H NMR (400 MHz, D₂O) δ 7.50-7.40 (m, 2H), | | | |
| | 7.17-7.06 (m, 2H), 4.37-4.23 (m, 1H), 3.84 (s, | | | |
| | 3H), 3.81 (br d, 1H), 3.43-3.17 (m, 3H), 3.03- | | | |
| | 2.91 (m, 1H), 2.58-2.47 (m, 1H), 2.46-2.34 (m, | | | |
| | 1H), 2.32-2.17 (m, 2H) | | | |
| 399 | 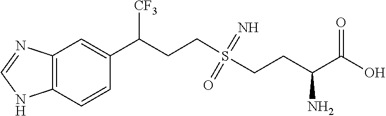 [SMILES: N=S(=O)(CCC(c1ccc2[nH]cnc2c1)C(F)(F)F)CC[C@H](N)C(=O)O] | 393.0 | 1.63 | B |
| | ¹H NMR (400 MHz, D₂O) δ 8.26 (s, 1H), 7.78- | | | |
| | 7.68 (m, 2H), 7.37 (d, 1H), 3.91-3.74 (m, 2H), | | | |
| | 3.43-3.21 (m, 3H), 3.03-2.91 (m, 1H), 2.67-2.42 | | | |
| | (m, 2H), 2.32-2.15 (m, 2H) | | | |
| 400 | 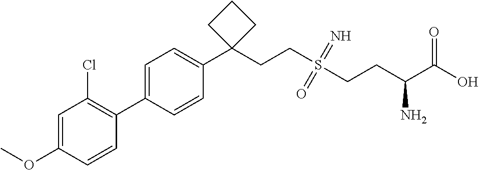 [SMILES: COc1ccc(-c2ccc(C3(CCS(=N)(=O)CC[C@H](N)C(=O)O)CCC3)cc2)c(Cl)c1] | 465.1 | 2.26 | C |
| | ¹H NMR (400 MHz, DMSO-d₆) δ 7.85-7.49 (m, | | | |
| | 1H), 7.37 (dd, 3H), 7.21 (d, 2H), 7.13 (d, 1H), 7.00 | | | |
| | (dd, 1H), 3.82 (s, 3H), 3.27 (br t, 1H), 3.23-3.16 | | | |
| | (m, 1H), 3.14-2.99 (m, 1H), 2.76-2.59 (m, 2H), | | | |
| | 2.40-2.29 (m, 2H), 2.25-1.91 (m, 7H), 1.88-1.74 | | | |
| | (m, 1H) | | | |
| 401 | 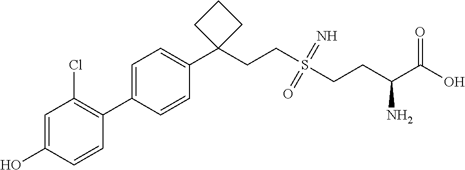 [SMILES: N=S(=O)(CC[C@H](N)C(=O)O)CCC1(c2ccc(-c3ccc(O)cc3Cl)cc2)CCC1] | 451.2 | 2.36 | B |
| | ¹H NMR (400 MHz, D₂O) δ 7.42 (d, 2H), 7.25 (dd, | | | |
| | 3H), 7.01 (d, 1H), 6.85 (dd, 1H), 3.76-3.68 (m, | | | |
| | 1H), 3.36-3.13 (m, 2H), 2.98-2.90 (m, 2H), 2.42- | | | |
| | 2.32 (m, 2H), 2.31-2.25 (m, 2H), 2.22-2.14 (m, | | | |
| | 4H), 2.11-2.02 (m, 1H), 1.88-1.77 (m, 1H) | | | |
| 402 | 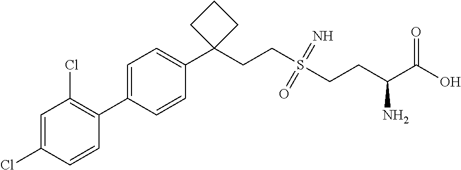 [SMILES: N=S(=O)(CC[C@H](N)C(=O)O)CCC1(c2ccc(-c3ccc(Cl)cc3Cl)cc2)CCC1] | 469.1 | 2.29 | C |
| | ¹H NMR (400 MHz, MeOD-d₄) δ 7.55 (d, 1H), 7.42- | | | |
| | 7.38 (m, 3H), 7.37-7.34 (m, 1H), 7.27 (d, 2H), | | | |
| | 3.69-3.61 (m, 1H), 3.40-3.33 (m, 1H), 3.29-3.22 | | | |
| | (m, 1H), 2.92-2.83 (m, 2H), 2.51-2.44 (m, 2H), | | | |
| | 2.37 (br t, 2H), 2.30-2.19 (m, 5H), 1.98-1.88 (m, | | | |
| | 1H) | | | |
| 403 | 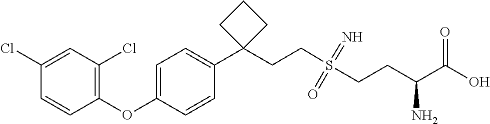 [SMILES: N=S(=O)(CC[C@H](N)C(=O)O)CCC1(c2ccc(Oc3ccc(Cl)cc3Cl)cc2)CCC1] | 485.1 | 2.40 | C |
| | ¹H NMR (400 MHz, MeOD-d4) δ 7.56 (d, 1H), | | | |
| | 7.31 (dd, 1H), 7.19 (d, 2H), 7.03-6.89 (m, 3H), | | | |
| | 3.67 (td, 1H), 3.30-3.15 (m, 2H), 2.93-2.74 (m, | | | |
| | 2H), 2.48-2.37 (m, 2H), 2.36-2.11 (m, 7H), 1.99- | | | |
| | 1.83 (m, 1H) | | | |

### Exemplary Embodiment 1aa32

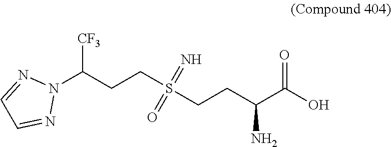 [SMILES: N=S(=O)(CCC(n1nccn1)C(F)(F)F)CC[C@H](N)C(=O)O]

### (2S)-2-amino-4-[[4,4,4-trifluoro-3-(triazol-2-yl)butyl]sulfonimidoyl]butanoic acid

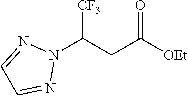 [SMILES: CCOC(=O)CC(n1nccn1)C(F)(F)F]

To a solution of ethyl (E)-4,4,4-trifluorobut-2-enoate (400 mg, 2.38 mmol, 1 eq) in acetonitrile (4 mL) was added 1H-1,2,3-triazole (246 mg, 3.57 mmol, 1.5 eq), DBU (1.09 g, 7.14 mmol, 1.08 mL, 3 eq), and the mixture was stirred at 60° C. for 16 h. The reaction was quenched with water (10 mL) and extracted with EtOAc (10 mL×2). The combined organic layers were washed with brine (10 mL), dried over Na₂SO₄, filtered, and concentrated under reduced pressure. The residue was purified by column chromatography (SiO₂, petroleum ether/EtOAc=99:1 to 9:1) to give ethyl 4,4,4-trifluoro-3-(triazol-2-yl)butanoate (190 mg, 801 μmol, 33.6% yield) as colorless oil. ¹H NMR (400 MHz, CDCl₃-d) δ 7.72 (s, 2H), 5.68 (ddd, 1H), 4.19-4.04 (m, 2H), 3.64 (dd, 1H), 3.15 (dd, 1H), 1.19 (t, 3H).

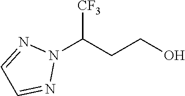 [SMILES: OCCC(n1nccn1)C(F)(F)F]

To a solution of ethyl 4,4,4-trifluoro-3-(triazol-2-yl)butanoate (190 mg, 801 μmol, 1 eq) in THE (2 mL) was added LiAlH₄ (30.4 mg, 801 μmol, 1 eq) at 0° C., and the mixture was stirred at 20° C. for 1 h. The reaction mixture was diluted with EtOAc, quenched with water (30 mL), 15% aq. NaOH (30 mL), water (90 mL), the mixture was filtered and concentrated to give 4,4,4-trifluoro-3-(triazol-2-yl)butan-1-ol (140 mg, 717 μmol, 89.5% yield) as colorless oil. ¹H NMR (400 MHz, CDCl₃-d) δ 7.74 (s, 2H), 5.48 (m, 1H), 3.82-3.72 (m, 1H), 3.27 (dt, 1H), 2.67 (tdd, 1H), 2.46-2.33 (m, 1H).

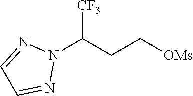 [SMILES: CS(=O)(=O)OCCC(n1nccn1)C(F)(F)F]

To a solution of 4,4,4-trifluoro-3-(triazol-2-yl)butan-1-ol (140 mg, 717 μmol, 1 eq) in DCM (2 mL) was added TEA (145 mg, 1.43 mmol, 199 μL, 2 eq) and methylsulfonyl methanesulfonate (187 mg, 1.08 mmol, 1.5 eq) at 0° C. The mixture was stirred at 20° C. for 1 h and the reaction was quenched by addition of ice-water (5 mL). The mixture was extracted with DCM (5 mL×2) and the combined organic layers were washed with brine (5 mL), dried over Na₂SO₄, filtered, and concentrated under reduced pressure to give 4,4,4-trifluoro-3-(2H-1,2,3-triazol-2-yl)butyl methanesulfonate (170 mg, 622 μmol, 86.7% yield) as colorless oil. ¹H NMR (400 MHz, CDCl₃-d) δ 7.77 (s, 2H), 5.38 (ddd, 1H), 4.40-4.31 (m, 1H), 3.81 (dtlH), 2.97 (s, 3H), 2.95-2.86 (m, 1H), 2.69-2.56 (m, 1H).

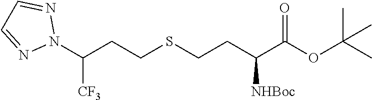 [SMILES: CC(C)(C)OC(=O)N[C@@H](CCSCCC(n1nccn1)C(F)(F)F)C(=O)OC(C)(C)C]

To a solution of tert-butyl (tert-butoxycarbonyl)-L-homocysteinate (181 mg, 622 μmol, 1 eq) in DMF (2 mL) was added 4,4,4-trifluoro-3-(2H-1,2,3-triazol-2-yl)butyl methanesulfonate (170 mg, 622 μmol, 1 eq), K₂CO₃ (257 mg, 1.87 mmol, 3 eq) and KI (206 mg, 1.24 mmol, 2 eq) in a glove box. The mixture was stirred at 20° C. for 16 h and the reaction was quenched with water (5 mL) and extracted with EtOAc (5 mL×2). The combined organic layers were washed with brine (5 mL), dried over Na₂SO₄, filtered, and concentrated under reduced pressure. The residue was purified by column chromatography (SiO₂, petroleum ether/EtOAc=19:1 to 7:3) to give tert-butyl 2-(tert-butoxycarbonylamino)-4-[4,4,4-trifluoro-3-(triazol-2-yl)butyl]sulfanyl-butanoate (235 mg, 501 μmol, 80.6% yield) as colorless oil. ¹H NMR (400 MHz, CDCl₃-d) δ 7.74 (d, 2H), 5.48-5.37 (m, 1H), 5.11 (br s, 1H), 4.30-4.19 (m, 1H), 2.89-2.77 (m, 1H), 2.58-2.47 (m, 3H), 2.38-2.25 (m, 1H), 2.21-2.12 (m, 1H), 2.07-1.99 (m, 1H), 1.88-1.75 (m, 1H), 1.46 (d, 18H).

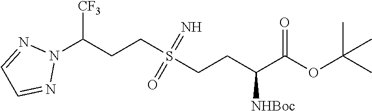 [SMILES: CC(C)(C)OC(=O)N[C@@H](CCS(=N)(=O)CCC(n1nccn1)C(F)(F)F)C(=O)OC(C)(C)C]

To a solution of tert-butyl 2-(tert-butoxycarbonylamino)-4-[4,4,4-trifluoro-3-(triazol-2-yl)butyl]sulfanyl-butanoate (235 mg, 501 μmol, 1 eq) in i-PrOH (3 mL) was added ammonium carbamate (234 mg, 3.01 mmol, 6 eq) and PhI(OAc)₂ (484 mg, 1.50 mmol, 3 eq). The mixture was stirred at 20° C. for 16 h. The reaction was quenched with water (10 mL) and extracted with EtOAc (10 mL×2). The combined organic layers were washed with brine (10 mL), dried over Na₂SO₄, filtered, and concentrated under reduced pressure. The residue was purified by column chromatography (SiO₂, petroleum ether/EtOAc=9:1 to 0:1) to give tert-butyl (2S)-2-(tert-butoxycarbonylamino)-4-[[4,4,4-trifluoro-3-(triazol-2-yl)butyl]sulfonimidoyl]butanoate (180 mg, 360 μmol, 71.8% yield) as colorless oil. ¹H NMR (400 MHz, CDCl₃-d) δ 7.78 (s, 2H), 5.57-5.42 (m, 1H), 5.31-5.15 (m, 1H), 4.33-4.20 (m, 1H), 3.24-3.11 (m, 1H), 3.09-2.65 (m, 6H), 2.42-2.27 (m, 1H), 1.47 (d, 18H).

A solution of tert-butyl (2S)-2-(tert-butoxycarbonylamino)-4-[[4,4,4-trifluoro-3-(triazol-2-yl)butyl]sulfonimidoyl]butanoate (180 mg, 360 μmol, 1 eq) in HCl/dioxane (20 mL, 4 M) was stirred at 20° C. for 16 h. The reaction mixture was concentrated under reduced pressure and the residue was purified by prep-HPLC (column: C18-1 (150×30 mm, 5 μm); mobile phase: [water (FA)-MeCN]; gradient: 10-40% B, 10 min) to give (2S)-2-amino-4-[[4,4,4-trifluoro-3-(triazol-2-yl)butyl]sulfonimidoyl]butanoic acid (57.0 mg, 146 μmol, 40.6% yield, FA) as white solid. LCMS: Rt=0.846 min, (ES⁺) m/z (M+H)⁺=344.0, HPLC Conditions: C. ¹H NMR (400 MHz, D₂O) δ 8.09-7.85 (m, 2H), 5.75-5.64 (m, 1H), 3.88-3.79 (m, 1H), 3.47-3.28 (m, 3H), 3.04-2.85 (m, 2H), 2.80-2.70 (m, 1H), 2.35-2.25 (m, 2H).

### Exemplary Embodiment 1aa33

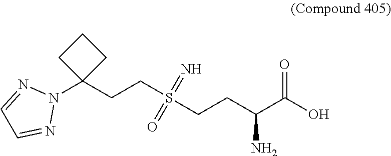 [SMILES: N=S(=O)(CC[C@H](N)C(=O)O)CCC1(n2nccn2)CCC1]

### (2S)-4-(2-(1-(2H-1,2,3-triazol-2-yl)cyclobutyl)ethylsulfonimidoyl)-2-aminobutanoic acid

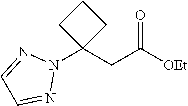 [SMILES: CCOC(=O)CC1(n2nccn2)CCC1]

A mixture of ethyl 2-cyclobutylideneacetate (500 mg, 3.57 mmol, 1 eq), 1H-triazole (369 mg, 5.35 mmol, 310.52 uL, 1.5 eq), and DBU (1.63 g, 10.7 mmol, 1.61 mL, 3 eq) in MeCN (8 mL) was stirred at 120° C. for 12 h in a 30-mL sealed tube. The reaction mixture was concentrated under reduced pressure and the residue was diluted with water (20 mL) and extracted with EtOAc (20 mL×2). The combined organic layers were washed with brine (10 mL), dried over Na₂SO₄, filtered, and concentrated under reduced pressure. The residue was purified by column chromatography (SiO₂, petroleum ether/EtOAc=100:1 to 100:8) to give ethyl 2-(1-(2H-1,2,3-triazol-2-yl)cyclobutyl)acetate (450 mg, 2.15 mmol, 60.29% yield) as a colorless oil. ¹H NMR (400 MHz, CDCl₃-d) δ 7.61 (s, 2H), 4.03 (q, 2H), 3.19 (s, 2H), 3.02-2.88 (m, 2H), 2.61-2.49 (m, 2H), 2.13-1.91 (m, 2H), 1.14 (t, 3H).

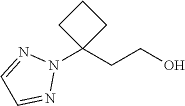 [SMILES: OCCC1(n2nccn2)CCC1]

To a solution of ethyl 2-(1-(2H-1,2,3-triazol-2-yl)cyclobutyl)acetate (450 mg, 2.15 mmol, 1 eq) in THE (5 mL) was added LAH (81.6 mg, 2.15 mmol, 1 eq) at 0° C. The mixture was stirred at 20° C. for 2 h. The mixture was diluted with EtOAc (10 mL), quenched with water (0.082 mL), 15% NaOH (82 mg) and water (0.246 mL), the mixture was filtered and concentrated to give 2-(1-(2H-1,2,3-triazol-2-yl)cyclobutyl)ethanol (330 mg, 1.97 mmol, 91.79% yield) as a yellow oil. ¹H NMR (400 MHz, CDCl₃-d) δ 7.68-7.58 (m, 2H), 3.53-3.44 (m, 2H), 2.97-2.81 (m, 2H), 2.52-2.44 (m, 2H), 2.37 (dt, 2H), 2.03-1.95 (m, 2H).

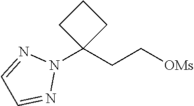 [SMILES: CS(=O)(=O)OCCC1(n2nccn2)CCC1]

To a solution of 2-(1-(2H-1,2,3-triazol-2-yl)cyclobutyl)ethanol (330 mg, 1.97 mmol, 1 eq) in DCM (6 mL) was added methylsulfonyl methanesulfonate (516 mg, 2.96 mmol, 1.5 eq) and TEA (599 mg, 5.92 mmol, 824 μL, 3 eq) at 0° C. The mixture was stirred at 20° C. for 2 h. The reaction mixture was quenched by addition of H₂O (5 mL) at 20° C. and extracted with DCM (4 mL×2). The combined organic layers were washed with brine (4 mL), dried over Na₂SO₄, filtered, and concentrated under reduced pressure to give a residue to give 2-(1-(2H-1,2,3-triazol-2-yl)cyclobutyl)ethyl methanesulfonate (410 mg, 1.67 mmol, 84.69% yield) as a yellow oil. ¹H NMR (400 MHz, CDCl₃-d) δ 7.66-7.61 (m, 2H), 4.04 (t, 2H), 2.98-2.86 (m, 5H), 2.60 (t, 2H), 2.51-2.42 (m, 2H), 2.10-1.97 (m, 2H).

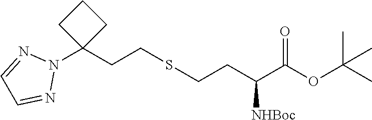 [SMILES: CC(C)(C)OC(=O)N[C@@H](CCSCCC1(n2nccn2)CCC1)C(=O)OC(C)(C)C]

To a solution of 2-(1-(2H-1,2,3-triazol-2-yl)cyclobutyl)ethyl methanesulfonate (231 mg, 944 μmol, 1.1 eq) in DMF (5 mL) was added tert-butyl (tert-butoxycarbonyl)-L-homocysteinate (250 mg, 858 μmol, 1 eq), KI (142 mg, 858 μmol, 1 eq), and K₂CO₃ (119 mg, 858 μmol, 1 eq) in a glove box. The mixture was stirred at 50° C. for 12 h, then diluted with H₂O (25 mL), and extracted with EtOAc (15 mL×3). The combined organic phase was washed with brine (10 mL), dried with anhydrous Na₂SO₄, filtered, and concentrated under reduced pressure. The residue was purified by column chromatography (SiO₂, petroleum ether/EtOAc=100:1 to 100:9) to give (S)-tert-butyl 4-((2-(1-(2H-1,2,3-triazol-2-yl)cyclobutyl)ethyl)thio)-2-((tert-butoxycarbonyl)amino)butanoate (296 mg, 671.81 μmol, 78.31% yield) as a yellow oil. ¹H NMR (400 MHz, CDCl₃-d) δ 7.63 (s, 2H), 5.09 (br d, 1H), 4.23 (br d, 1H), 2.92-2.80 (m, 1H), 2.52-2.47 (m, 2H), 2.44-2.33 (m, 4H), 2.24-2.15 (m, 2H), 2.04-1.91 (m, 4H), 1.46 (d, 18H).

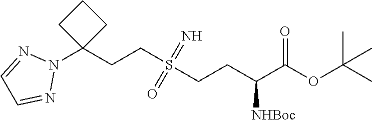 [SMILES: CC(C)(C)OC(=O)N[C@@H](CCS(=N)(=O)CCC1(n2nccn2)CCC1)C(=O)OC(C)(C)C]

To a solution of (S)-tert-butyl 4-((2-(1-(2H-1,2,3-triazol-2-yl)cyclobutyl)ethyl)thio)-2-((tert-butoxycarbonyl)amino)butanoate (296 mg, 671.81 μmol, 1 eq) in i-PrOH (10 mL) was added ammonium carbamate (210 mg, 2.69 mmol, 4 eq) and PhI(Oac)₂ (433 mg, 1.34 mmol, 2 eq). The mixture was stirred at 20° C. for 12 h and then concentrated under reduced pressure. The residue was diluted with water (5 mL) and extracted with EtOAc (5 mL×2). The combined organic layers were washed with brine (3 mL), dried over Na₂SO₄, filtered, and concentrated under reduced pressure. The residue was purified by column chromatography (SiO₂, petroleum ether/EtOAc=100:1 to 2:1 to give (2S)-tert-butyl 4-(2-(1-(2H-1,2,3-triazol-2-yl)cyclobutyl)ethylsulfonimidoyl)-2-((tert-butoxycarbonyl)amino)butanoate (200 mg, 424.08 μmol, 63.12% yield) as a yellow oil. ¹H NMR (400 MHz, CDCl₃-d) δ 7.65 (s, 2H), 5.31 (s, 1H), 5.24 (br d, 1H), 4.26 (br d, 1H), 3.22-3.00 (m, 2H), 2.98-2.80 (m, 4H), 2.73-2.63 (m, 2H), 2.42-2.25 (m, 3H), 2.10-1.99 (m, 3H), 1.47 (d, 18H).

A solution of (2S)-tert-butyl 4-(2-(1-(2H-1,2,3-triazol-2-yl)cyclobutyl)ethylsulfonimidoyl)-2-((tert-butoxycarbonyl)amino)butanoate (200 mg, 424.08 μmol, 1 eq) in HCl/dioxane (20 mL) was stirred at 20° C. for 12 h. The reaction mixture was concentrated under reduced pressure and the residue was purified by prep-HPLC (column: Waters Xbridge Prep OBD C18 (150×40 mm, 10 μm); mobile phase: [water (NH₄HCO₃)-MeCN]; B: 1-20%, 8 min) to give (2S)-4-(2-(1-(2H-1,2,3-triazol-2-yl)cyclobutyl)ethylsulfonimidoyl)-2-aminobutanoic acid (33.88 mg, 106.21 μmol, 25.05% yield) as a white solid. LCMS: Rt=2.488 min., (ES⁺) m/z (M+H)⁺=316.2, HPLC Conditions: G. ¹H NMR (400 MHz, D₂O) δ 7.76 (s, 2H), 3.80-3.68 (m, 1H), 3.42-3.12 (m, 2H), 2.93-2.84 (m, 2H), 2.80-2.68 (m, 2H), 2.58-2.50 (m, 2H), 2.43-2.32 (m, 2H), 2.24-2.15 (m, 2H), 2.07-1.87 (m, 2H).

The compounds described in Table 17 were prepared using the general methods outlined above.

TABLE 17
Characterization of Compounds 406-423

| Compound | | MS | HPLC | HPLC |
|---|---|---|---|---|
| # | Structure | (M + H)⁺ | Rt | cond. |
| 406 | 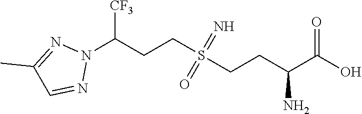 [SMILES: Cc1cnn(C(CCS(=N)(=O)CC[C@H](N)C(=O)O)C(F)(F)F)n1] | 358.0 | 2.24 | A |
| | ¹H NMR (400 MHz, D₂O) δ 7.71 (s, 1H), 5.64- | | | |
| | 5.46 (m, 1H), 3.91-3.75 (m, 1H), 3.53-3.21 (m, | | | |
| | 3H), 3.05-2.94 (m, 1H), 2.92-2.79 (m, 1H), 2.77- | | | |
| | 2.66 (m, 1H), 2.34-2.24 (m, 5H) | | | |
| 407 | 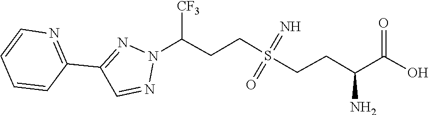 [SMILES: N=S(=O)(CCC(n1ncc(-c2ccccn2)n1)C(F)(F)F)CC[C@H](N)C(=O)O] | 421.1 | 1.63 | C |
| | ¹H NMR (400 MHz, D₂O) δ 8.58 (d, 1H), 8.37 (s, | | | |
| | 1H), 7.97 (d, 2H), 7.52-7.46 (m, 1H), 5.81-5.69 | | | |
| | (m, 1H), 3.83 (tt, 1H), 3.50-3.31 (m, 3H), 3.17- | | | |
| | 2.94 (m, 2H), 2.86-2.74 (m, 1H), 2.35-2.25 (m, | | | |
| | 2H). | | | |
| 408 | 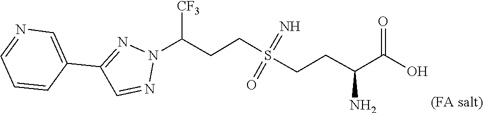 [SMILES: N=S(=O)(CCC(n1ncc(-c2cccnc2)n1)C(F)(F)F)CC[C@H](N)C(=O)O] | 421.1 | 1.28 | C |
| | ¹H NMR (400 MHz, D₂O) δ 7.47 (d, 2H), 7.36 (d, | | | |
| | 2H), 7.28-7.20 (m, 2H), 7.00 (d, 1H), 3.72-3.66 | | | |
| | (m, 1H), 3.65-3.55 (m, 1H), 3.38-3.27 (m, 1H), | | | |
| | 3.21 (br s, 1H), 3.09 (td, 1H), 2.94-2.76 (m, 1H), | | | |
| | 2.60-2.46 (m, 1H), 2.46-2.35 (m, 1H), 2.30- | | | |
| | 2.12 (m, 2H) | | | |
| 409 | 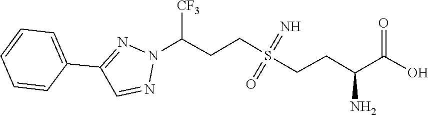 [SMILES: N=S(=O)(CCC(n1ncc(-c2ccccc2)n1)C(F)(F)F)CC[C@H](N)C(=O)O] | 420.2 | 2.25 | B |
| | ¹H NMR (400 MHz, D₂O) δ 8.22 (s, 1H), 7.80 (br | | | |
| | d, 2H), 7.58-7.33 (m, 3H), 5.72-5.55 (m, 1H), | | | |
| | 3.78 (td, 1H), 3.49-3.17 (m, 3H), 3.10-2.87 (m, | | | |
| | 2H), 2.80-2.64 (m, 1H), 2.40-2.10 (m, 2H) | | | |
| 410 | 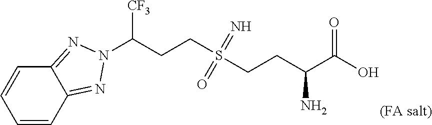 [SMILES: N=S(=O)(CCC(n1nc2ccccc2n1)C(F)(F)F)CC[C@H](N)C(=O)O] | 394.1 | 1.78 | C |
| | ¹H NMR (400 MHz, D₂O) δ 7.97-7.80 (m, 2H), | | | |
| | 7.52 (dd, 2H), 6.05-5.83 (m, 1H), 3.84-3.69 (m, | | | |
| | 1H), 3.48-3.16 (m, 3H), 3.11-2.75 (m, 3H), 2.32- | | | |
| | 2.12 (m, 2H) | | | |
| 411 | 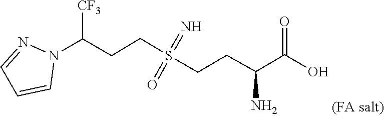 [SMILES: N=S(=O)(CCC(n1cccn1)C(F)(F)F)CC[C@H](N)C(=O)O] | 343.0 | 0.88 | C |
| | ¹H NMR (400 MHz, D₂O) δ 7.85 (d, 1H), 7.73 (s, | | | |
| | 1H), 6.49 (s, 1H), 5.34-5.23 (m, 1H), 3.83 (td, | | | |
| | 1H), 3.45-3.23 (m, 3H), 3.00-2.89 (m, 1H), 2.83- | | | |
| | 2.61 (m, 2H), 2.33-2.24 (m, 2H) | | | |
| 412 | 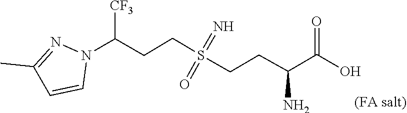 [SMILES: Cc1ccn(C(CCS(=N)(=O)CC[C@H](N)C(=O)O)C(F)(F)F)n1] | 357.1 | 1.64 | B |
| | ¹H NMR (400 MHz, D₂O) δ 7.70 (d, 1H), 6.32- | | | |
| | 6.23 (m, 1H), 5.21-5.10 (m, 1H), 3.88-3.80 (m, | | | |
| | 1H), 3.47-3.19 (m, 3H), 3.01-2.89 (m, 1H), 2.77- | | | |
| | 2.57 (m, 2H), 2.34-2.27 (m, 2H), 2.24 (s, 3H) | | | |
| 413 | 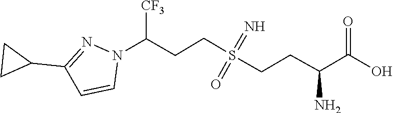 [SMILES: N=S(=O)(CCC(n1ccc(C2CC2)n1)C(F)(F)F)CC[C@H](N)C(=O)O] | 383.1 | 2.83 | A |
| | ¹H NMR (400 MHz, D₂O) δ 7.69 (br s, 1H), 6.09 | | | |
| | (s, 1H), 5.19-5.05 (m, 1H), 3.90-3.75 (m, 1H), | | | |
| | 3.47-3.18 (m, 3H), 3.04-2.88 (m, 1H), 2.77- | | | |
| | 2.56 (m, 2H), 2.36-2.19 (m, 2H), 1.98-1.84 (m, | | | |
| | 1H), 0.95 (br d, 2H), 0.74-0.57 (m, 2H) | | | |
| 414 | 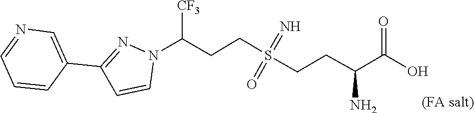 [SMILES: N=S(=O)(CCC(n1ccc(-c2cccnc2)n1)C(F)(F)F)CC[C@H](N)C(=O)O] | 420.1 | 1.26 | C |
| | ¹H NMR (400 MHz, D₂O) δ 8.92 (s, 1H), 8.52 (d, | | | |
| | 1H), 8.26 (br d, 1H), 7.95 (d, 1H), 7.57 (dd, 1H), | | | |
| | 6.90 (d, 1H), 5.41-5.26 (m, 1H), 3.82 (q, 1H), | | | |
| | 3.48-3.26 (m, 3H), 3.12-2.98 (m, 1H), 2.96- | | | |
| | 2.80 (m, 1H), 2.78-2.63 (m, 1H), 2.37-2.21 (m, | | | |
| | 2H) | | | |
| 415 | 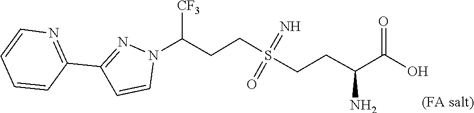 [SMILES: N=S(=O)(CCC(n1ccc(-c2ccccn2)n1)C(F)(F)F)CC[C@H](N)C(=O)O] | 420.1 | 1.37 | C |
| | ¹H NMR (400 MHz, D₂O) δ 8.57 (br d, 1H), 8.39 - | | | |
| | 8.27 (m, 1H), 8.22 (br t, 1H), 8.09 (br d, 1H), 7.96 | | | |
| | (br d, 1H), 7.65 (br t, 1H), 7.03 (br d, 1H), 5.44- | | | |
| | 5.28 (m, 1H), 3.77 (q, 1H), 3.50-3.20 (m, 3H), | | | |
| | 3.11-2.97 (m, 1H), 2.95-2.79 (m, 1H), 2.73- | | | |
| | 2.60 (m, 1H), 2.32-2.17 (m, 2H) | | | |
| 416 | 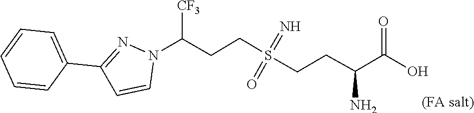 [SMILES: N=S(=O)(CCC(n1ccc(-c2ccccc2)n1)C(F)(F)F)CC[C@H](N)C(=O)O] | 419.0 | 3.51 | A |
| | ¹H NMR (400 MHz, D₂O) δ 7.95 (s, 1H), 7.87- | | | |
| | 7.75 (m, 2H), 7.61-7.42 (m, 3H), 6.94-6.82 (m, | | | |
| | 1H), 5.52-5.16 (m, 1H), 3.91-3.78 (m, 1H), 3.55- | | | |
| | 3.28 (m, 3H), 3.16-3.01 (m, 1H), 2.98-2.83 | | | |
| | (m, 1H), 2.81-2.66 (m, 1H), 2.43-2.21 (m, 2H) | | | |
| 417 | 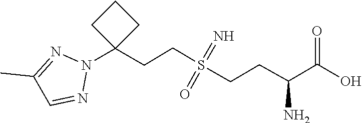 [SMILES: Cc1cnn(C2(CCS(=N)(=O)CC[C@H](N)C(=O)O)CCC2)n1] | 330.1 | 2.31 | A |
| | ¹H NMR (400 MHz, D₂O) δ 7.56 (s, 1H), 3.92- | | | |
| | 3.76 (m, 1H), 3.46-3.16 (m, 2H), 2.96-2.85 (m, | | | |
| | 2H), 2.81-2.66 (m, 2H), 2.58-2.48 (m, 2H), 2.43- | | | |
| | 2.32 (m, 1H), 2.46-2.15 (m, 1H), 2.08-1.89 | | | |
| | (m, 2H) | | | |
| 418 | 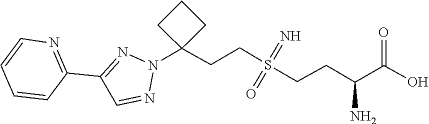 [SMILES: N=S(=O)(CC[C@H](N)C(=O)O)CCC1(n2ncc(-c3ccccn3)n2)CCC1] | 393.1 | 1.51 | C |
| | ¹H NMR (400 MHz, D₂O) δ 8.55 (br d, 1H), 8.22 | | | |
| | (s, 1H), 7.99-7.88 (m, 2H), 7.45 (dt, 1H), 3.86- | | | |
| | 3.76 (m, 1H), 3.45-3.23 (m, 2H), 3.08-3.00 (m, | | | |
| | 2H), 2.91-2.81 (m, 2H), 2.70-2.61 (m, 2H), 2.50- | | | |
| | 2.41 (m, 2H), 2.32-2.21 (m, 2H), 2.12-1.95 | | | |
| | (m, 2H). | | | |
| 419 | 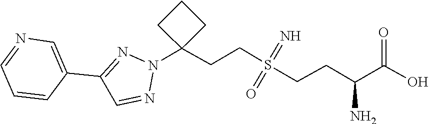 [SMILES: N=S(=O)(CC[C@H](N)C(=O)O)CCC1(n2ncc(-c3cccnc3)n2)CCC1] | 393.2 | 2.16 | T |
| | ¹H NMR (400 MHz, D₂O) δ 8.94 (br s, 1H), 8.19 | | | |
| | (s, 1H), 7.65-7.55 (m, 1H), 3.87-3.75 (m, 1H), | | | |
| | 3.47-3.22 (m, 2H), 3.03 (br d, 2H), 2.91-2.79 | | | |
| | (m, 2H), 2.70-2.59 (m, 2H), 2.51-2.40 (m, 2H), | | | |
| | 2.27 (q, 2H), 2.13-1.94 (m, 2H) | | | |
| 420 | 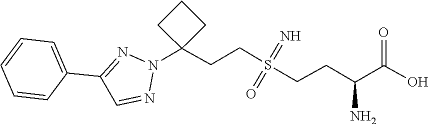 [SMILES: N=S(=O)(CC[C@H](N)C(=O)O)CCC1(n2ncc(-c3ccccc3)n2)CCC1] | 392.2 | 1.93 | C |
| | ¹H NMR (400 MHz, D₂O) δ 8.06 (s, 1H), 7.75 (d, | | | |
| | 2H), 7.53-7.37 (m, 3H), 3.78-3.67 (m, 1H), 3.41- | | | |
| | 3.16 (m, 2H), 3.03-2.91 (m, 2H), 2.86-2.73 | | | |
| | (m, 2H), 2.65-2.55 (m, 2H), 2.40 (dt, 2H), 2.19 | | | |
| | (dtd, 2H), 2.08-1.87 (m, 2H) | | | |
| 421 | 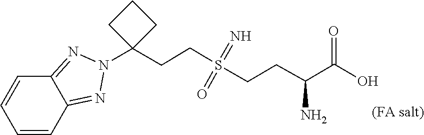 [SMILES: N=S(=O)(CC[C@H](N)C(=O)O)CCC1(n2nc3ccccc3n2)CCC1] | 366.0 | 2.83 | A |
| | ¹H NMR (400 MHz, D₂O) δ 7.93-7.83 (m, 2H), | | | |
| | 7.62-7.42 (m, 2H), 3.78 (dt, 1H), 3.47-3.09 (m, | | | |
| | 2H), 3.03-2.89 (m, 4H), 2.77-2.69 (m, 2H), 2.64- | | | |
| | 2.50 (m, 2H), 2.26-1.98 (m, 4H) | | | |
| 422 | 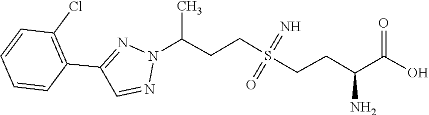 [SMILES: CC(CCS(=N)(=O)CC[C@H](N)C(=O)O)n1ncc(-c2ccccc2Cl)n1] | 454.1 | 2.00 | C |
| | ¹H NMR (400 MHz, MeOD-d₄) δ 8.35 (s, 1H), 7.94- | | | |
| | 7.86 (m, 1H), 7.59-7.52 (m, 1H), 7.47-7.38 | | | |
| | (m, 2H), 5.87-5.72 (m, 1H), 3.71 (td, 1H), 3.49- | | | |
| | 3.34 (m, 2H), 3.30-3.21 (m, 1H), 3.13-2.91 (m, | | | |
| | 2H), 2.81-2.68 (m, 1H), 2.42-2.23 (m, 2H). | | | |
| 423 | 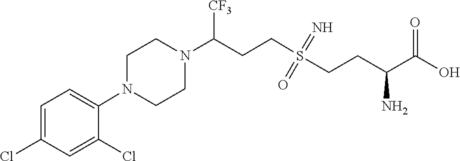 [SMILES: N=S(=O)(CCC(N1CCN(c2ccc(Cl)cc2Cl)CC1)C(F)(F)F)CC[C@H](N)C(=O)O] | 505.0 | 2.68 | B |
| | ¹H NMR (400 MHz, MeOD-d₄) δ 7.40 (d, 1H), 7.27 | | | |
| | (dd, 1H), 7.11 (d, 1H), 3.74-3.68 (m, 1H), 3.55- | | | |
| | 3.48 (m, 1H), 3.47-3.39 (m, 2H), 3.38-3.33 (m, | | | |
| | 2H), 3.16-3.11 (m, 2H), 3.07-3.01 (m, 2H), 3.00- | | | |
| | 2.93 (m, 2H), 2.82 (br d, 2H), 2.40-2.33 (m, | | | |
| | 2H), 2.24-2.11 (m, 2H). | | | |

### Exemplary Embodiment 1aa34

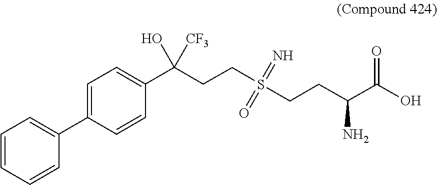 [SMILES: N=S(=O)(CC[C@H](N)C(=O)O)CCC(O)(c1ccc(-c2ccccc2)cc1)C(F)(F)F]

### (2S)-4-(3-([1,1′-biphenyl]-4-yl)-4,4,4-trifluoro-3-hydroxybutylsulfonimidoyl)-2-aminobutanoic acid

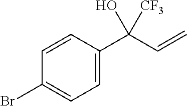

To a solution of 1-(4-bromophenyl)-2,2,2-trifluoro-ethanone (10 g, 39.5 mmol, 6.02 mL, 1 eq) in THE (100 mL) was added bromo(vinyl)magnesium (1 M, 51.4 mL, 1.3 eq) at −65° C. The mixture was stirred at 0° C. for 1.5 h under N₂. The reaction mixture was poured into aq. NH₄Cl solution (300 mL) and extracted with EtOAc (80 mL×3). The combined organic layers were washed with brine (50 mL), dried over Na₂SO₄, filtered, and concentrated under reduced pressure. The residue was purified by silica gel chromatography (petroleum ether/EtOAc=1:0 to 10:1) to give 2-(4-bromophenyl)-1,1,1-trifluorobut-3-en-2-ol (11 g, 39.14 mmol, 99.02% yield) as a colorless oil. ¹H NMR (400 MHz, CDCl₃-d)) δ 7.56-7.51 (m, 2H), 7.49-7.45 (m, 2H), 7.27 (s, 1H), 6.41 (dd, 1H), 5.67-5.49 (m, 2H), 2.59 (s, 1H).

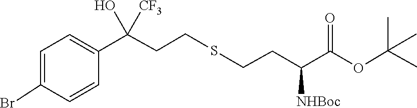 [SMILES: CC(C)(C)OC(=O)N[C@@H](CCSCCC(O)(c1ccc(Br)cc1)C(F)(F)F)C(=O)OC(C)(C)C]

To a solution of 2-(4-bromophenyl)-1,1,1-trifluorobut-3-en-2-ol (1.5 g, 5.34 mmol, 1 eq) and tert-butyl (2S)-2-(tert-butoxycarbonylamino)-4-sulfanyl-butanoate (1.87 g, 6.40 mmol, 1.2 eq) in MeOH (25.5 mL) and H₂O (25.5 mL) was added AIBN (87.6 mg, 534 μmol, 0.1 eq). The mixture was stirred at 60° C. for 18 h under Ar. The reaction mixture was poured into water (100 mL) and extracted with EtOAc (50 mL×3). The combined organic layers were washed with brine (30 mL), dried over Na₂SO₄, filtered, and concentrated under reduced pressure. The residue was purified by silica gel chromatography (petroleum ether/EtOAc=1:0 to 10:1) to give tert-butyl S-(3-(4-bromophenyl)-4,4,4-trifluoro-3-hydroxybutyl)-N-(tert-butoxycarbonyl)-L-homocysteinate (1.3 g, 2.27 mmol, 42.55% yield) as a colorless oil. ¹H NMR (400 MHz, CDCl₃-d) δ 7.60-7.52 (m, 2H), 7.50-7.41 (m, 2H), 5.25-5.10 (m, 1H), 4.34-4.19 (m, 1H), 4.10-3.97 (m, 1H), 2.64-2.30 (m, 6H), 2.01-1.79 (m, 2H), 1.48-1.45 (m, 18H).

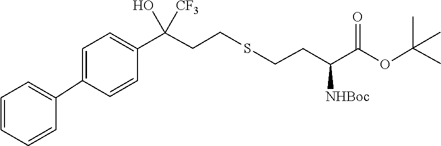 [SMILES: CC(C)(C)OC(=O)N[C@@H](CCSCCC(O)(c1ccc(-c2ccccc2)cc1)C(F)(F)F)C(=O)OC(C)(C)C]

To a solution of tert-butyl S-(3-(4-bromophenyl)-4,4,4-trifluoro-3-hydroxybutyl)-N-(tert-butoxycarbonyl)-L-homocysteinate (0.5 g, 873 μmol, 1 eq) and phenylboronic acid (138 mg, 1.14 mmol, 1.3 eq) in dioxane (5 mL) and H₂O (5 mL) was added K₂CO₃ (362 mg, 2.62 mmol, 3 eq) and Pd(dppf)Cl₂ (128 mg, 175 μmol, 0.2 eq). The mixture was stirred at 100° C. for 6 h and then the suspension was filtered through a pad of Celite, and the filter cake was washed with EtOAc (30 mL×3). The combined organic layers were washed with brine (20 mL×2), dried over Na₂SO₄, filtered, and concentrated under reduced pressure to give a residue. The residue was purified by silica gel chromatography (petroleum ether/EtOAc=1:0 to 5:1) to give tert-butyl S-(3-([1,1′-biphenyl]-4-yl)-4,4,4-trifluoro-3-hydroxybutyl)-N-(tert-butoxycarbonyl)-L-homocysteinate (0.45 g, 789.92 μmol, 90.44% yield) as a colorless oil. ¹H NMR (400 MHz, CDCl₃-d) δ 7.66-7.59 (m, 6H), 7.46 (t, 2H), 7.40-7.34 (m, 1H), 5.25-5.10 (m, 1H), 4.38-4.21 (m, 1H), 2.66-2.49 (m, 4H), 2.47-2.35 (m, 2H), 2.01-1.81 (m, 2H), 1.47-1.45 (m, 18H).

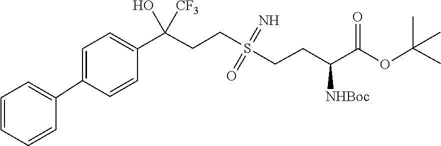 [SMILES: CC(C)(C)OC(=O)N[C@@H](CCS(=N)(=O)CCC(O)(c1ccc(-c2ccccc2)cc1)C(F)(F)F)C(=O)OC(C)(C)C]

To a solution of tert-butyl S-(3-([1,1′-biphenyl]-4-yl)-4,4,4-trifluoro-3-hydroxybutyl)-N-(tert-butoxycarbonyl)-L-homocysteinate (0.5 g, 878 μmol, 1 eq) in i-PrOH (12 mL) was added PhI(OAc)₂ (1.13 g, 3.51 mmol, 4 eq) and ammonium carbamate (548 mg, 7.02 mmol, 8 eq). The mixture was stirred at 25° C. for 3 h. The reaction mixture was poured into water (50 mL) and extracted with EtOAc (30 mL×3). The combined organic layers were washed with brine (30 mL), dried over Na₂SO₄, filtered, and concentrated under reduced pressure. The residue was purified by silica gel chromatography (petroleum ether/EtOAc=1:0 to 1:2) to give tert-butyl (2S)-4-(3-([1,1′-biphenyl]-4-yl)-4,4,4-trifluoro-3-hydroxybutylsulfonimidoyl)-2-((tert-butoxycarbonyl)amino)butanoate (0.4 g, 665.90 μmol, 75.87% yield) as a white solid. ¹H NMR (400 MHz, CDCl₃-d) δ 7.71-7.58 (m, 6H), 7.46 (t, 2H), 7.40-7.35 (m, 1H), 5.31-5.14 (m, 1H), 4.34-4.20 (m, 1H), 3.33-3.20 (m, 1H), 3.19-3.00 (m, 3H), 2.99-2.88 (m, 1H), 2.87-2.62 (m, 2H), 2.45-2.28 (m, 1H), 2.15-2.07 (m, 1H), 1.49 (d, 6H), 1.47-1.41 (m, 12H).

To a solution of give tert-butyl (2S)-4-(3-([1,1′-biphenyl]-4-yl)-4,4,4-trifluoro-3-hydroxybutylsulfonimidoyl)-2-((tert-butoxycarbonyl)amino)butanoate (0.34 g, 566 μmol, 1 eq) in dioxane (0.5 mL) was added HCl/dioxane (4 M, 5 mL, 35.3 eq) at 0° C. The mixture was stirred at 25° C. for 3 h under N₂. The reaction mixture was concentrated under reduced pressure to give a residue. The residue was purified by prep-HPLC (column: Waters Xbridge BEH C18 (100×30 mm, 10 μm); mobile phase: [water (NH₄HCO₃)-MeCN]; gradient: 15-45% B, 8 min) to give (2S)-4-(3-([1,1′-biphenyl]-4-yl)-4,4,4-trifluoro-3-hydroxybutylsulfonimidoyl)-2-aminobutanoic acid (99.2 mg, 220.09 μmol, 29.64% yield) as a white solid. LCMS: Rt=2.106 min, (ES⁺) m/z (M+H)⁺=445.1, HPLC Conditions: C. ¹H NMR (400 MHz, MeOD-d₄) δ 7.69 (s, 4H), 7.67-7.62 (m, 2H), 7.45 (t, 2H), 7.39-7.32 (m, 1H), 3.73-3.62 (m, 1H), 3.43-3.33 (m, 1H), 3.28-3.10 (m, 2H), 2.88-2.71 (m, 2H), 2.69-2.52 (m, 1H), 2.37-2.19 (m, 2H).

### Exemplary Embodiment 1aa35

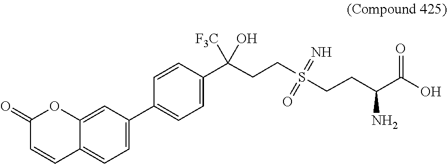 [SMILES: N=S(=O)(CC[C@H](N)C(=O)O)CCC(O)(c1ccc(-c2ccc3ccc(=O)oc3c2)cc1)C(F)(F)F]

### (2S)-2-amino-4-(4,4,4-trifluoro-3-hydroxy-3-(4-(2-oxo-2H-chromen-7-yl)phenyl)butylsulfonimidoyl)butanoic acid

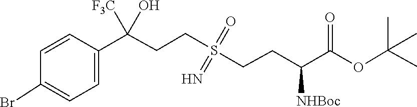 [SMILES: CC(C)(C)OC(=O)N[C@@H](CCS(=N)(=O)CCC(O)(c1ccc(Br)cc1)C(F)(F)F)C(=O)OC(C)(C)C]

To a solution of tert-butyl (2S)-4-[3-(4-bromophenyl)-4,4,4-trifluoro-3-hydroxy-butyl]sulfanyl-2-(tert-butoxycarbonylamino)butanoate (367 mg, 641 μmol, 1 eq) in i-PrOH (6 mL) was added ammonium carbamate (400 mg, 5.13 mmol, 8 eq) and PhI(OAc)₂ (826 mg, 2.56 mmol, 4 eq). The mixture was stirred at 25° C. for 6 h and then concentrated under reduced pressure. The residue was diluted with water (5 mL) and extracted with EtOAc (10 mL×2). The combined organic layers were washed with brine (2 mL), dried over Na₂SO₄, filtered, and concentrated under reduced pressure. The residue was purified by prep-TLC (SiO₂, petroleum ether/EtOAc=1:1) to give tert-butyl (2S)-4-(3-(4-bromophenyl)-4,4,4-trifluoro-3-hydroxybutylsulfonimidoyl)-2-((tert-butoxycarbonyl)amino)butanoate (283 mg, 469 μmol, 73.2% yield) as a yellow oil. ¹H NMR (400 MHz, CDCl₃-d) δ 7.62-7.41 (m, 4H), 4.34-4.21 (m, 1H), 3.40-3.27 (m, 1H), 3.27-3.10 (m, 2H), 3.04-2.88 (m, 1H), 2.76-2.64 (m, 2H), 2.48-2.26 (m, 2H), 1.51-1.42 (m, 18H).

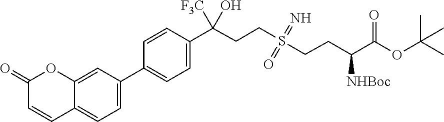 [SMILES: CC(C)(C)OC(=O)N[C@@H](CCS(=N)(=O)CCC(O)(c1ccc(-c2ccc3ccc(=O)oc3c2)cc1)C(F)(F)F)C(=O)OC(C)(C)C]

A mixture of 7-(4,4,5,5-tetramethyl-1,3,2-dioxaborolan-2-yl)chromen-2-one (45.1 mg, 165.7 μmol, 2 eq), tert-butyl (2S)-4-(3-(4-bromophenyl)-4,4,4-trifluoro-3-hydroxybutylsulfonimidoyl)-2-((tert-butoxycarbonyl)amino)butanoate (50 mg, 82.9 μmol, 1 eq), Na₂CO₃ (26.3 mg, 249 μmol, 3 eq) and Pd(dppf)Cl₂ (6.77 mg, 8.28 μmol, 0.1 eq) in t-BuOH (0.9 mL) and H₂O (0.3 mL) was degassed and purged 3 times with N₂, and then the mixture was stirred at 90° C. for 1.5 h under N₂ atmosphere. The reaction mixture was diluted with H₂O (5 mL) and extracted with EtOAc (5 mL×3). The combined organic phase was washed with brine (3 mL), dried with anhydrous Na₂SO₄, filtered, and concentrated under reduced pressure. The residue was purified by prep-TLC (SiO₂, petroleum ether/EtOAc=1:1) to give tert-butyl (2S)-2-((tert-butoxycarbonyl)amino)-4-(4,4,4-trifluoro-3-hydroxy-3-(4-(2-oxo-2H-chromen-7-yl)phenyl)butylsulfonimidoyl)butanoate (78 mg, crude) as a yellow oil. ¹H NMR (400 MHz, CDCl₃-d) δ 7.84-7.66 (m, 5H), 7.60-7.48 (m, 3H), 5.41-5.28 (m, 1H), 4.35-4.18 (m, 1H), 3.49-3.24 (m, 3H), 2.89-2.73 (m, 2H), 2.51-2.28 (m, 2H), 1.51-1.39 (m, 18H).

A solution of tert-butyl (2S)-2-((tert-butoxycarbonyl)amino)-4-(4,4,4-trifluoro-3-hydroxy-3-(4-(2-oxo-2H-chromen-7-yl)phenyl)butylsulfonimidoyl)butanoate (58 mg, 86.7 μmol, 1 eq) in HCl/dioxane (4 M, 4 mL) was stirred at 20° C. for 12 h. The reaction mixture was concentrated under reduced pressure and the residue was purified by prep-HPLC (column: Waters Xbridge Prep OBD C18 (150×40 mm, 10 μm); mobile phase: [water (NH₄HCO₃)-MeCN]; gradient: 10-50% B over 8 min) to give (2S)-2-amino-4-(4,4,4-trifluoro-3-hydroxy-3-(4-(2-oxo-2H-chromen-7-yl)phenyl)butylsulfonimidoyl)butanoic acid (15.53 mg, 29.17 μmol, 33.63% yield) as a white solid. LCMS: Rt=2.077 min., (ES⁺) m/z (M+H)⁺=513.1, HPLC Conditions: C. ¹H NMR (400 MHz, MeOD-d₄) δ=8.00 (d, 1H), 7.85-7.61 (m, 7H), 6.46 (d, 1H), 3.74-3.62 (m, 1H), 3.44-3.34 (m, 1H), 3.28-3.18 (m, 2H), 2.86-2.71 (m, 2H), 2.68-2.54 (m, 1H), 2.27 (br d, 2H)

### Exemplary Embodiment 1aa36

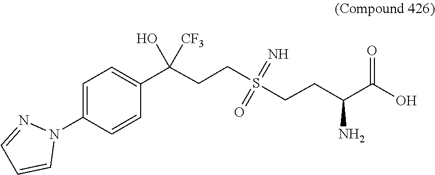 [SMILES: N=S(=O)(CC[C@H](N)C(=O)O)CCC(O)(c1ccc(-n2cccn2)cc1)C(F)(F)F]

### (2S)-4-(3-(4-(1H-pyrazol-1-yl) phenyl)-4, 4, 4-trifluoro-3-hydroxybutylsulfonimidoyl)-2-aminobutanoic acid

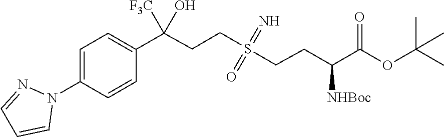 [SMILES: CC(C)(C)OC(=O)N[C@@H](CCS(=N)(=O)CCC(O)(c1ccc(-n2cccn2)cc1)C(F)(F)F)C(=O)OC(C)(C)C]

A mixture of tert-butyl (2S)-4-(3-(4-bromophenyl)-4,4,4-trifluoro-3-hydroxybutylsulfonimidoyl)-2-((tert-butoxycarbonyl)amino)butanoate (100 mg, 165.70 μmol, 1 eq), 1H-pyrazole (112.81 mg, 1.66 mmol, 10 eq), Cs₂CO₃ (107.98 mg, 331.41 μmol, 2 eq), bis[tris(dibenzylideneacetone)palladium(0)](151.74 mg, 165.70 μmol, 1 eq) and di-tert-butyl-[2-(1,3,5-triphenylpyrazol-4-yl)pyrazol-3-yl]phosphane (16.79 mg, 33.14 μmol, 0.2 eq) in 1,4-dioxane (3 mL) was degassed and purged 3 times with N₂. The solution was heated to 100° C. under N₂ atmosphere and stirred for 1 h. The mixture was poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM/MeOH=10:1) to give tert-butyl (2S)-4-(3-(4-(1H-pyrazol-1-yl)phenyl)-4,4,4-trifluoro-3-hydroxybutylsulfonimidoyl)-2-((tert-butoxycarbonyl)amino)butanoate (41.1 mg, 69.58 μmol, 41.99% yield) as a white solid. ¹H NMR (400 MHz, CDCl₃-d) δ 8.01-7.93 (m, 1H), 7.78-7.74 (m, 2H), 7.72-7.67 (m, 2H), 7.35-7.28 (m, 1H), 7.25-7.20 (m, 1H), 7.20-7.14 (m, 1H), 6.50 (s, 1H), 5.35-5.12 (m, 1H), 4.35-4.11 (m, 1H), 3.16-3.05 (m, 3H), 3.02-2.92 (m, 3H), 2.17-1.99 (m, 2H), 1.49-1.42 (m, 18H), 1.32-1.20 (m, 1H)

A solution of tert-butyl (2S)-4-(3-(4-(1H-pyrazol-1-yl)phenyl)-4,4,4-trifluoro-3-hydroxybutylsulfonimidoyl)-2-((tert-butoxycarbonyl)amino)butanoate (41.1 mg, 69.58 μmol, 1 eq) in HCl/dioxane (6 M, 20 mL) was stirred at 25° C. for 4 h. The mixture was concentrated and the residue was purified by prep-HPLC (column: Waters Xbridge Prep OBD C18 (150×40 mm, 10 μm); mobile phase: [water (NH₄HCO₃)-MeCN]; gradient: 5-35% B over 8 min) to give (2S)-4-(3-(4-(1H-pyrazol-1-yl) phenyl)-4, 4, 4-trifluoro-3-hydroxybutylsulfonimidoyl)-2-aminobutanoic acid (8 mg, 16.94 μmol, 24.34% yield) as a white solid. LCMS: Rt=1.417 min, (ES⁺) m/z (M+H)⁺=435.1, HPLC Conditions: C. ¹H NMR (400 MHz, MeOD-d₄) δ 8.28 (d, 1H), 7.87-7.81 (m, 2H), 7.78-7.71 (m, 3H), 6.55 (t, 1H), 3.73-3.62 (m, 1H), 3.44-3.33 (m, 1H), 3.28-3.11 (m, 2H), 2.84-2.70 (m, 2H), 2.66-2.53 (m, 1H), 2.32-2.15 (m, 2H).

### Exemplary Embodiment 1aa37

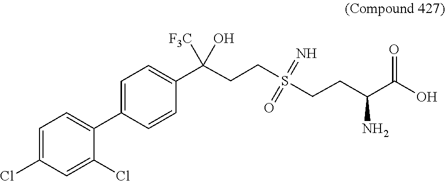 [SMILES: N=S(=O)(CC[C@H](N)C(=O)O)CCC(O)(c1ccc(-c2ccc(Cl)cc2Cl)cc1)C(F)(F)F]

### (2S)-2-amino-4-((3-(2′, 4′-dichloro-[1, 1′-biphenyl]-4-yl)-4, 4, 4-trifluoro-3-hydroxybutyl) sulfonyl) butanoic acid

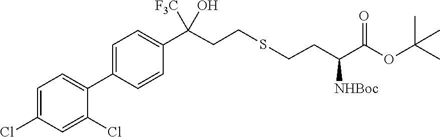 [SMILES: CC(C)(C)OC(=O)N[C@@H](CCSCCC(O)(c1ccc(-c2ccc(Cl)cc2Cl)cc1)C(F)(F)F)C(=O)OC(C)(C)C]

To a solution of tert-butyl (2S)-4-(3-(4-bromophenyl)-4,4,4-trifluoro-3-hydroxybutylsulfonimidoyl)-2-((tert-butoxycarbonyl)amino)butanoate (80 mg, 140 μmol, 1 eq) in t-BuOH (1.5 mL) and H₂O (0.5 mL) was added (2, 4-dichlorophenyl)boronic acid (40.0 mg, 210 μmol, 1.5 eq), Pd(dppf)Cl₂ (11.4 mg, 14.0 μmol, 0.1 eq) and sodium carbonate (44.4 mg, 419 μmol, 3 eq). The mixture was stirred at 90° C. for 2 hr. The mixture was poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM:MeOH=10:1) to give tert-butyl N-(tert-butoxycarbonyl)-S-(3-(2′, 4′-dichloro-[1, 1′-biphenyl]-4-yl)-4, 4, 4-trifluoro-3-hydroxybutyl)-L-homocysteinate (73.6 mg, 115 μmol, 82.5% yield) as a colorless oil.

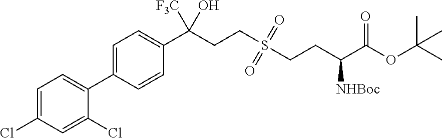 [SMILES: CC(C)(C)OC(=O)N[C@@H](CCS(=O)(=O)CCC(O)(c1ccc(-c2ccc(Cl)cc2Cl)cc1)C(F)(F)F)C(=O)OC(C)(C)C]

To a solution of give tert-butyl N-(tert-butoxycarbonyl)-S-(3-(2′, 4′-dichloro-[1, 1′-biphenyl]-4-yl)-4, 4, 4-trifluoro-3-hydroxybutyl)-L-homocysteinate (73.6 mg, 116 μmol, 1 eq) in DCM (2 mL) was added m-CPBA (46.8 mg, 231 μmol, 85% purity, 2 eq) at 0° C. The mixture was stirred at 20° C. for 1 hr. The mixture was poured into H₂O (20 mL) and extracted with EtOAc (20 mL×3). The combined organic layers were dried over Na₂SO₄, filtered, and concentrated under reduced pressure. The residue was purified by prep-TLC (SiO₂, DCM:MeOH=10:1) to give tert-butyl (2S)-2-((tert-butoxycarbonyl) amino)-4-((3-(2′, 4′-dichloro-[1, 1′-biphenyl]-4-yl)-4, 4, 4-trifluoro-3-hydroxybutyl) sulfonyl) butanoate (57 mg, 85.0 μmol, 73.8% yield) as a colorless oil.

A solution of tert-butyl (2S)-2-((tert-butoxycarbonyl) amino)-4-((3-(2′, 4′-dichloro-[1, 1′-biphenyl]-4-yl)-4, 4, 4-trifluoro-3-hydroxybutyl) sulfonyl) butanoate (57 mg, 85.0 μmol, 1 eq) in HCl/dioxane (5 M, 10 mL) was stirred at 30° C. for 4 h. The mixture was concentrated and the residue was purified by prep-HPLC (column: Waters Xbridge C18 (150×50 mm, 10 μm); mobile phase: [H₂O (10 mM NH₄HCO₃)-MeCN]; gradient: 25-55% B over 8.0 min) to give (2S)-2-amino-4-((3-(2′, 4′-dichloro-[1, 1′-biphenyl]-4-yl)-4, 4, 4-trifluoro-3-hydroxybutyl) sulfonyl) butanoic acid (11.3 mg, 21.9 μmol, 25.8% yield) as a white solid. LCMS: Rt=2.334 min, (ES⁺) m/z (M+H)⁺=514.1, HPLC Conditions: C. ¹H NMR (400 MHz, MeOD-d₄) δ 7.70 (br d, 2H), 7.59 (d, 1H), 7.52 (d, 2H), 7.45-7.36 (m, 2H), 3.69 (dt, 1H), 3.40-3.34 (m, 1H), 3.28-3.14 (m, 2H), 2.81-2.66 (m, 2H), 2.64-2.51 (m, 1H), 2.32-2.20 (m, 2H).

The compounds described in Table 18 were prepared using the general methods

TABLE 18
Characterization of Compounds 428-458

| Compound | | MS | HPLC | HPLC |
|---|---|---|---|---|
| # | Structure | (M + H)⁺ | Rt | cond. |
| 428 | 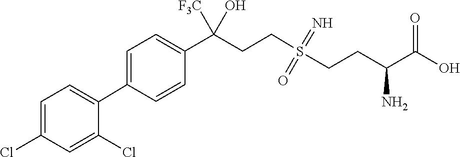 [SMILES: N=S(=O)(CC[C@H](N)C(=O)O)CCC(O)(c1ccc(-c2ccc(Cl)cc2Cl)cc1)C(F)(F)F] | 513.0 | 2.65 | B |
| | ¹H NMR (400 MHz, MeOD-d₄) δ 7.71 (d, 2H), 7.58 | | | |
| | (d, 1H), 7.51 (d, 2H), 7.44-7.36 (m, 2H), 3.73- | | | |
| | 3.62 (m, 1H), 3.45-3.33 (m, 1H), 3.26-3.16 (m, | | | |
| | 1H), 2.87-2.74 (m, 2H), 2.67-2.53 (m, 1H), 2.36- | | | |
| | 2.23 (m, 2H) | | | |
| 429 | 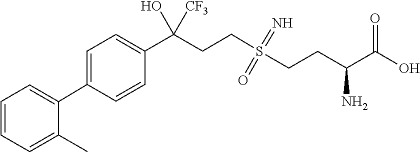 [SMILES: Cc1ccccc1-c1ccc(C(O)(CCS(=N)(=O)CC[C@H](N)C(=O)O)C(F)(F)F)cc1] | 459.1 | 2.20 | C |
| | ¹H NMR (400 MHz, MeOD-d₄) δ 7.68 (d, 2H), 7.39 | | | |
| | (d, 2H), 7.31-7.15 (m, 4H), 3.74-3.61 (m, 1H), | | | |
| | 3.51-3.35 (m, 1H), 3.30-3.14 (m, 2H), 2.88-2.72 | | | |
| | (m, 2H), 2.67-2.54 (m, 1H), 2.35-2.27 (m, 2H), | | | |
| | 2.26 (s, 3H) | | | |
| 430 | 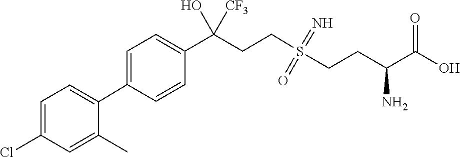 [SMILES: Cc1cc(Cl)ccc1-c1ccc(C(O)(CCS(=N)(=O)CC[C@H](N)C(=O)O)C(F)(F)F)cc1] | 493.1 | 2.32 | C |
| | ¹H NMR (400 MHz, MeOD-d₄) δ 7.69 (d, 2H), 7.39 | | | |
| | (d, 2H), 7.31 (d, 1H), 7.23 (d, 1H), 7.20 (d, 1H), | | | |
| | 3.69 (br d, 1H), 3.46-3.34 (m, 1H), 3.29-3.15 (m, | | | |
| | 2H), 2.89-2.73 (m, 2H), 2.67-2.53 (m, 1H), 2.29 | | | |
| | (br d, 2H), 2.25 (s, 3H) | | | |
| 431 | 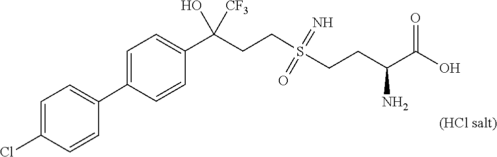 [SMILES: N=S(=O)(CC[C@H](N)C(=O)O)CCC(O)(c1ccc(-c2ccc(Cl)cc2)cc1)C(F)(F)F] | 479.1 | 2.263 | C |
| | ¹H NMR (400 MHz, MeOD-d₄) δ 7.70 (s, 4H), 7.65 | | | |
| | (dd, 2H), 7.48-7.44 (m, 2H), 3.72-3.63 (m, 1H), | | | |
| | 3.43-3.33 (m, 1H), 3.29-3.17 (m, 2H), 2.85-2.71 | | | |
| | (m, 2H), 2.59 (br dd, 1H), 2.33-2.21 (m, 2H) | | | |
| 432 | 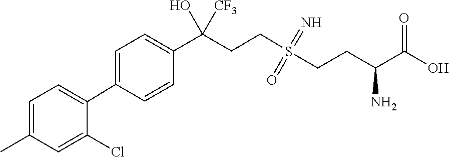 [SMILES: Cc1ccc(-c2ccc(C(O)(CCS(=N)(=O)CC[C@H](N)C(=O)O)C(F)(F)F)cc2)c(Cl)c1] | 493.1 | 2.59 | B |
| | ¹H NMR (400 MHz, MeOD-d₄) δ 7.68 (d, 2H), 7.49 | | | |
| | (d, 2H), 7.33 (s, 1H), 7.29-7.23 (m, 1H), 7.22- | | | |
| | 7.17 (m, 1H), 3.75-3.64 (m, 1H), 3.43-3.34 (m, | | | |
| | 1H), 3.27-3.14 (m, 2H), 2.90-2.73 (m, 2H), 2.68- | | | |
| | 2.51 (m, 1H), 2.38 (s, 3H), 2.34-2.25 (m, 2H) | | | |
| 433 | 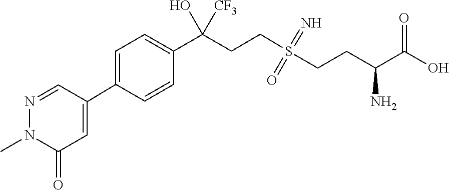 [SMILES: Cn1ncc(-c2ccc(C(O)(CCS(=N)(=O)CC[C@H](N)C(=O)O)C(F)(F)F)cc2)cc1=O] | 477.1 | 1.73 | C |
| | ¹H NMR (400 MHz, MeOD-d₄) δ 8.33 (s, 1H), 7.87- | | | |
| | 7.76 (m, 4H), 7.20 (t, 1H), 3.82 (s, 3H), 3.73- | | | |
| | 3.62 (m, 1H), 3.43-3.34 (m, 1H), 3.28-3.14 (m, | | | |
| | 2H), 2.85-2.68 (m, 2H), 2.66-2.55 (m, 1H), 2.34- | | | |
| | 2.20 (m, 2H) | | | |
| 434 | 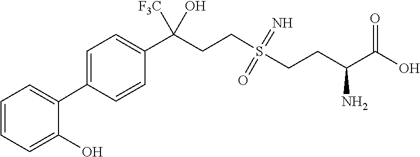 [SMILES: N=S(=O)(CC[C@H](N)C(=O)O)CCC(O)(c1ccc(-c2ccccc2O)cc1)C(F)(F)F] | 461.1 | 2.00 | C |
| | ¹H NMR (400 MHz, MeOD-d₄) δ 7.63 (s, 4H), 7.29- | | | |
| | 7.25 (m, 1H), 7.19-7.13 (m, 1H), 6.93-6.88 (m, | | | |
| | 2H), 3.71-3.64 (m, 1H), 3.42-3.33 (m, 1H), 3.29- | | | |
| | 3.18 (m, 2H), 2.86-2.73 (m, 2H), 2.63-2.54 (m, | | | |
| | 1H), 2.33-2.23 (m, 2H) | | | |
| 435 | 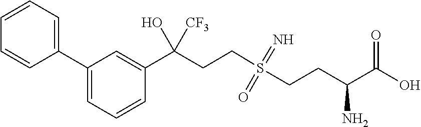 [SMILES: N=S(=O)(CC[C@H](N)C(=O)O)CCC(O)(c1cccc(-c2ccccc2)c1)C(F)(F)F] | 445.2 | 2.03 | C |
| | ¹H NMR (400 MHz, MeOD-d₄) δ 7.87 (s, 1H), 7.68- | | | |
| | 7.57 (m, 4H), 7.55-7.49 (m, 1H), 7.45 (t, 2H), | | | |
| | 7.39-7.32 (m, 1H), 3.72-3.61 (m, 1H), 3.44-3.33 | | | |
| | (m, 1H), 3.29-3.13 (m, 2H), 2.89-2.71 (m, 2H), | | | |
| | 2.68-2.55 (m, 1H), 2.33-2.21 (m, 2H) | | | |
| 436 | 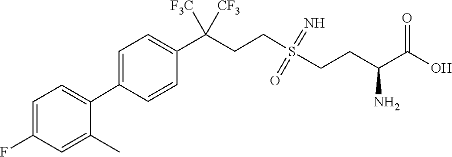 [SMILES: Cc1cc(F)ccc1-c1ccc(C(CCS(=N)(=O)CC[C@H](N)C(=O)O)(C(F)(F)F)C(F)(F)F)cc1] | 477.1 | 2.21 | C |
| | ¹H NMR (400 MHz, MeOD-d₄) δ 7.68 (d, 2H), 7.38 | | | |
| | (d, 2H), 7.21 (ddd, 1H), 7.04 (dd, 1H), 6.97 (dt, | | | |
| | 1H), 3.75-3.65 (m, 1H), 3.44-3.35 (m, 1H), 3.29- | | | |
| | 3.16 (m, 2H), 2.86-2.73 (m, 2H), 2.66-2.53 (m, | | | |
| | 1H), 2.35-2.24 (m, 5H) | | | |
| 437 | 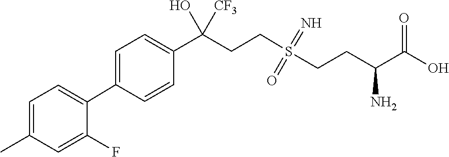 [SMILES: Cc1ccc(-c2ccc(C(O)(CCS(=N)(=O)CC[C@H](N)C(=O)O)C(F)(F)F)cc2)c(F)c1] | 477.1 | 2.21 | C |
| | ¹H NMR (400 MHz, MeOD-d₄) δ 7.75-7.66 (m, | | | |
| | 2H), 7.64-7.50 (m, 2H), 7.39 (dt, 1H), 7.15-6.95 | | | |
| | (m, 2H), 3.78-3.63 (m, 1H), 3.46-3.34 (m, 1H), | | | |
| | 3.30-3.13 (m, 2H), 2.87-2.70 (m, 2H), 2.66-2.51 | | | |
| | (m, 1H), 2.39 (s, 3H), 2.29 (td, 2H) | | | |
| 438 | 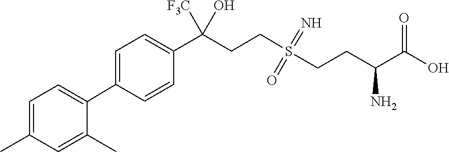 [SMILES: Cc1ccc(-c2ccc(C(O)(CCS(=N)(=O)CC[C@H](N)C(=O)O)C(F)(F)F)cc2)c(C)c1] | 473.2 | 2.23 | C |
| | 1H NMR (400 MHz, MeOD-d₄) δ 7.66 (d, 2H), | | | |
| | 7.37 (d, 2H), 7.11-7.00 (m, 3H), 3.73-3.64 (m, | | | |
| | 1H), 3.44-3.34 (m, 1H), 3.29-3.19 (m, 2H), 2.91- | | | |
| | 2.68 (m, 2H), 2.68-2.54 (m, 1H), 2.34 (s, 3H), | | | |
| | 2.31-2.27 (m, 2H), 2.23 (s, 3H) | | | |
| 439 | 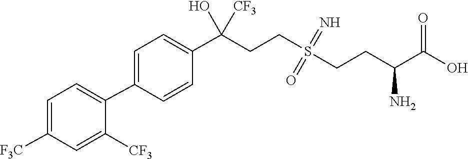 [SMILES: N=S(=O)(CC[C@H](N)C(=O)O)CCC(O)(c1ccc(-c2ccc(C(F)(F)F)cc2C(F)(F)F)cc1)C(F)(F)F] | 581.1 | 2.42 | C |
| | ¹H NMR (400 MHz, MeOD-d₄) δ 8.07 (s, 1H), 8.00 | | | |
| | (d, 1H), 7.73 (d, 2H), 7.64 (dd, 1H), 7.44 (d, 2H), | | | |
| | 3.73-3.64 (m, 1H), 3.43-3.34 (m, 1H), 3.27 (br d, | | | |
| | 2H), 2.86-2.75 (m, 2H), 2.67-2.56 (m, 1H), 2.30 | | | |
| | (q, 2H) | | | |
| 440 | 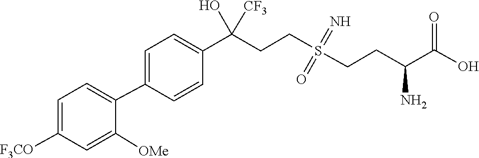 [SMILES: COc1cc(OC(F)(F)F)ccc1-c1ccc(C(O)(CCS(=N)(=O)CC[C@H](N)C(=O)O)C(F)(F)F)cc1] | 559.0 | 2.71 | B |
| | ¹H NMR (400 MHz, MeOD-d₄) δ 7.65 (br d, 2H), | | | |
| | 7.60-7.51 (m, 2H), 7.39 (br d, 1H), 7.01-6.93 (m, | | | |
| | 2H), 3.84 (s, 3H), 3.73-3.64 (m, 1H), 3.52-3.39 | | | |
| | (m, 1H), 3.28-3.15 (m, 2H), 2.80 (br t, 2H), 2.66- | | | |
| | 2.52 (m, 1H), 2.37-2.18 (m, 2H) | | | |
| 441 | 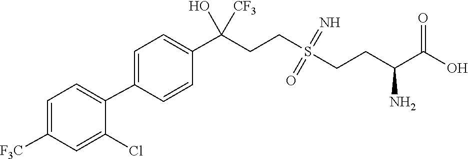 [SMILES: N=S(=O)(CC[C@H](N)C(=O)O)CCC(O)(c1ccc(-c2ccc(C(F)(F)F)cc2Cl)cc1)C(F)(F)F] | 547.0 | 2.70 | B |
| | ¹H NMR (400 MHz, MeOH-d₄) δ 7.84 (s, 1H), 7.75 | | | |
| | (d, 2H), 7.71 (d, 1H), 7.61 (br d, 1H), 7.56 (d, 2H), | | | |
| | 3.72-3.64 (m, 1H), 3.44-3.35 (m, 1H), 3.26 (br d, | | | |
| | 2H), 2.86-2.75 (m, 2H), 2.67-2.55 (m, 1H), 2.33- | | | |
| | 2.24 (m, 2H) | | | |
| 442 | 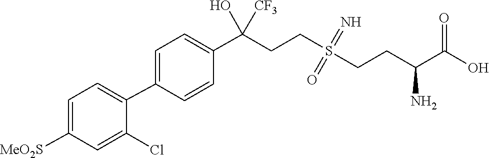 [SMILES: CS(=O)(=O)c1ccc(-c2ccc(C(O)(CCS(=N)(=O)CC[C@H](N)C(=O)O)C(F)(F)F)cc2)c(Cl)c1] | 557.0 | 2.02 | C |
| | ¹H NMR (400 MHz, MeOD-d₄) δ 8.11 (d, H), 7.96 | | | |
| | (dd, 1H), 7.76 (br d, 2H), 7.67 (dd, 1H), 7.57 (d, | | | |
| | 2H), 3.74-3.62 (m, 1H), 3.42-3.34 (m, 2H), 3.27 | | | |
| | (br s, 1H), 3.20 (s, 3H), 2.87-2.73 (m, 2H), 2.70- | | | |
| | 2.55 (m, 1H), 2.36-2.22 (m, 2H) | | | |
| 443 | 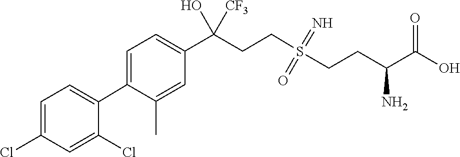 [SMILES: Cc1cc(C(O)(CCS(=N)(=O)CC[C@H](N)C(=O)O)C(F)(F)F)ccc1-c1ccc(Cl)cc1Cl] | 526.9. | 2.74 | B |
| | ¹H NMR (400 MHz, MeOD-d₄) δ 7.58 (br d, 2H), | | | |
| | 7.50 (br dd, 1H), 7.44-7.38 (m, 1H), 7.25 (dd, 1H), | | | |
| | 7.18 (d, 1H), 3.72-3.64 (m, 1H), 3.39 (br s, 1H), | | | |
| | 3.28-3.10 (m, 2H), 2.79 (br s, 2H), 2.68-2.52 (m, | | | |
| | 1H), 2.30 (br dd, 2H), 2.14 (s, 3H) | | | |
| 444 | 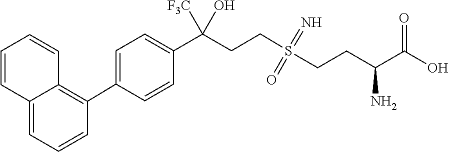 [SMILES: N=S(=O)(CC[C@H](N)C(=O)O)CCC(O)(c1ccc(-c2cccc3ccccc23)cc1)C(F)(F)F] | 495.1 | 2.30 | C |
| | ¹H NMR (400 MHz, MeOD-d4) δ 7.96-7.82 (m, | | | |
| | 3H), 7.77 (d, 2H), 7.59-7.40 (m, 6H), 3.74-3.64 | | | |
| | (m, 1H), 3.50-3.33 (m, 2H), 3.29-3.20 (m, 1H), | | | |
| | 2.97-2.76 (m, 2H), 2.71-2.58 (m, 1H), 2.39-2.24 | | | |
| | (m, 2H) | | | |
| 445 | 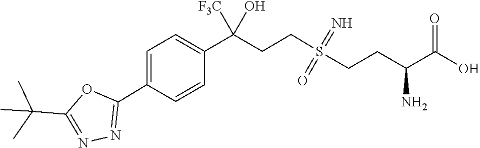 [SMILES: CC(C)(C)c1nnc(-c2ccc(C(O)(CCS(=N)(=O)CC[C@H](N)C(=O)O)C(F)(F)F)cc2)o1] | 493.2 | 0.356 | C |
| | ¹H NMR (400 MHz, MeOD-d₄) δ 8.11 (d, 2H), 7.85 | | | |
| | (d, 2H), 3.71-3.63 (m, 1H), 3.42-3.35 (m, 1H), | | | |
| | 3.30-3.11 (m, 2H), 2.85-2.56 (m, 3H), 2.32-2.21 | | | |
| | (m, 2H), 1.50 (s, 9H) | | | |
| 446 | 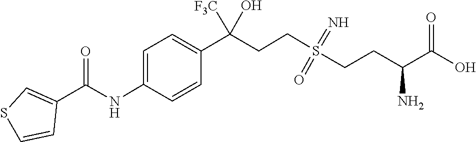 [SMILES: N=S(=O)(CC[C@H](N)C(=O)O)CCC(O)(c1ccc(NC(=O)c2ccsc2)cc1)C(F)(F)F] | 494.0 | 2.10 | B |
| | ¹H NMR (400 MHz, MeOD-d₄) δ 8.25-8.25 (m, | | | |
| | 1H), 7.78 (s, 1H), 7.76 (s, 1H), 7.64 (dd, 1H), 7.62 | | | |
| | (s, 1H), 7.60 (s, 1H), 7.53 (dd, 1H), 3.69-3.64 (m, | | | |
| | 1H), 3.37 (br d, 1H), 3.27-3.10 (m, 2H), 2.81- | | | |
| | 2.71 (m, 2H), 2.61-2.51 (m, 1H), 2.30-2.22 (m, | | | |
| | 2H). | | | |
| 447 | 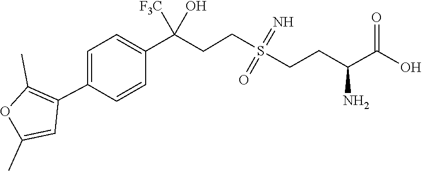 [SMILES: Cc1cc(-c2ccc(C(O)(CCS(=N)(=O)CC[C@H](N)C(=O)O)C(F)(F)F)cc2)c(C)o1] | 463.1 | 2.45 | B |
| | ¹H NMR (400 MHz, MeOD-d₄) δ 7.61 (d, 2H), 7.48- | | | |
| | 7.40 (m, 2H), 6.18 (s, 1H), 3.70-3.63 (m, 1H), | | | |
| | 3.45-3.34 (m, 1H), 3.27-3.14 (m, 2H), 2.81-2.70 | | | |
| | (m, 2H), 2.62-2.51 (m, 1H), 2.40 (s, 3H), 2.26 (s, | | | |
| | 5H) | | | |
| 448 | 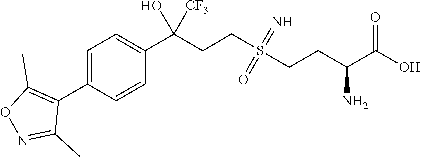 [SMILES: Cc1noc(C)c1-c1ccc(C(O)(CCS(=N)(=O)CC[C@H](N)C(=O)O)C(F)(F)F)cc1] | 464.1 | 1.93 | C |
| | ¹H NMR (400 MHz, MeOD-d₄) δ 7.73 (d, 2H), 7.44 | | | |
| | (d, 2H), 3.74-3.63 (m, 1H), 3.44-3.34 (m, 1H), | | | |
| | 3.29-3.16 (m, 2H), 2.85-2.72 (m, 2H), 2.67-2.54 | | | |
| | (m, 1H), 2.43 (s, 3H), 2.34-2.24 (m, 5H) | | | |
| 449 | 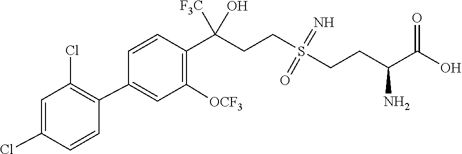 [SMILES: N=S(=O)(CC[C@H](N)C(=O)O)CCC(O)(c1ccc(-c2ccc(Cl)cc2Cl)cc1OC(F)(F)F)C(F)(F)F] | 597.1 | 2.44 | C |
| | ¹H NMR (400 MHz, MeOD-d₄) δ 8.07 (d, 1H), 7.65 | | | |
| | (d, 1H), 7.53-7.39 (m, 4H), 3.78-3.67 (m, 1H), | | | |
| | 3.38 (br d, 2H), 3.29-3.12 (m, 2H), 3.00-2.82 (m, | | | |
| | 1H), 2.69-2.52 (m, 1H), 2.39-2.27 (m, 2H) | | | |
| 450 | 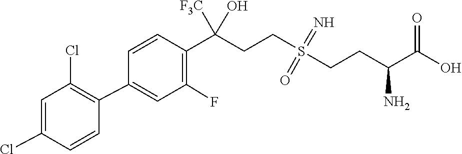 [SMILES: N=S(=O)(CC[C@H](N)C(=O)O)CCC(O)(c1ccc(-c2ccc(Cl)cc2Cl)cc1F)C(F)(F)F] | 530.9 | 2.71 | B |
| | ¹H NMR (400 MHz, MeOD-d₄) δ 7.89 (t, 1H), 7.61 | | | |
| | (d, 1H), 7.47-7.21 (m, 4H), 3.81-3.62 (m, 1H), | | | |
| | 3.40 (br s, 1H), 3.29-3.18 (m, 2H), 3.17-3.03 (m, | | | |
| | 1H), 2.99-2.79 (m, 1H), 2.65-2.49 (m, 1H), 2.38- | | | |
| | 2.21 (m, 2H) | | | |
| 451 | 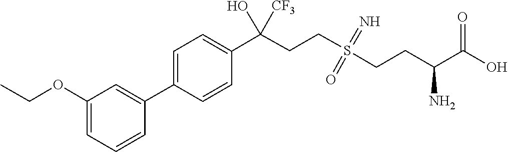 [SMILES: CCOc1cccc(-c2ccc(C(O)(CCS(=N)(=O)CC[C@H](N)C(=O)O)C(F)(F)F)cc2)c1] | 489.2 | 2.13 | C |
| | ¹H NMR (400 MHz, MeOD-d₄ + DMSO-d) δ = 7.71 | | | |
| | (s, 4H), 7.41-7.34 (m, 1H), 7.26-7.16 (m, 2H), | | | |
| | 6.94 (dd, 1H), 4.12 (q, 2H), 3.67 (br d, 1H), 3.46- | | | |
| | 3.35 (m, 1H), 3.28-3.12 (m, 2H), 2.86-2.72 (m, | | | |
| | 2H), 2.59 (br s, 1H), 2.35-2.21 (m, 2H), 1.43 (t,). | | | |
| 452 | 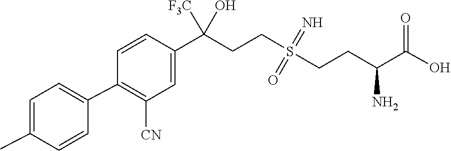 [SMILES: Cc1ccc(-c2ccc(C(O)(CCS(=N)(=O)CC[C@H](N)C(=O)O)C(F)(F)F)cc2C#N)cc1] | 484.2 | 2.11 | C |
| | ¹H NMR (400 MHz, MeOD-d₄) δ 8.07 (s, 1H), 7.95 | | | |
| | (br d, 1H), 7.66 (d, 1H), 7.54-7.45 (m, 2H), 7.34 | | | |
| | (d, 2H), 3.74-3.63 (m, 1H), 3.46-3.34 (m, 1H), | | | |
| | 3.29-3.15 (m, 2H), 2.88-2.71 (m, 2H), 2.70-2.54 | | | |
| | (m, 1H), 2.43 (s, 3H), 2.34-2.23 (m, 2H) | | | |
| 453 | 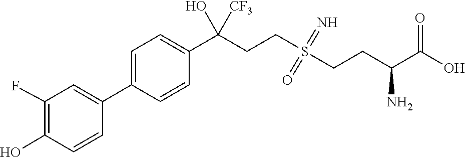 [SMILES: N=S(=O)(CC[C@H](N)C(=O)O)CCC(O)(c1ccc(-c2ccc(O)c(F)c2)cc1)C(F)(F)F] | 479.1 | 2.66 | G |
| | ¹H NMR (400 MHz, MeOD-d₄) δ 7.68-7.61 (m, | | | |
| | 4H), 7.38 (td, 1H), 7.30 (br d, 1H), 6.99 (t, 1H), | | | |
| | 3.70-3.61 (m, 1H), 3.42-3.33 (m, 1H), 3.29-3.14 | | | |
| | (m, 2H), 2.84-2.70 (m, 2H), 2.65-2.52 (m, 1H), | | | |
| | 2.33-2.21 (m, 2H). | | | |
| 454 | 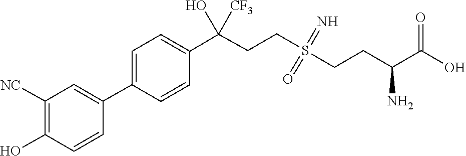 [SMILES: N#Cc1cc(-c2ccc(C(O)(CCS(=N)(=O)CC[C@H](N)C(=O)O)C(F)(F)F)cc2)ccc1O] | 486.1 | 2.15 | G |
| | ¹H NMR (400 MHz, MeOD-d₄) δ 7.81-7.73 (m, | | | |
| | 2H), 7.70-7.63 (m, 4H), 7.02 (d, 1H), 3.69-3.61 | | | |
| | (m, 1H), 3.43-3.33 (m, 1H), 3.29-3.16 (m, 2H), | | | |
| | 2.83-2.70 (m, 2H), 2.65-2.52 (m, 1H), 2.32-2.20 | | | |
| | (m, 2H) | | | |
| 455 | 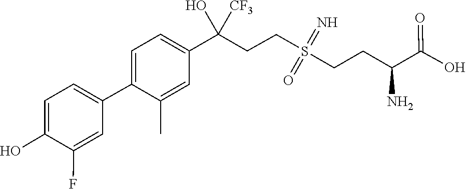 [SMILES: Cc1cc(C(O)(CCS(=N)(=O)CC[C@H](N)C(=O)O)C(F)(F)F)ccc1-c1ccc(O)c(F)c1] | 493.1 | 1.92 | C |
| | ¹H NMR (400 MHz, MeOD-d₄) δ = 7.51 (s, 1H), | | | |
| | 7.44 (br d, 1H), 7.24 (d, 1H), 7.06-6.93 (m, 3H), | | | |
| | 3.72-3.63 (m, 1H), 3.39 (br s, 1H), 3.28-3.11 (m, | | | |
| | 2H), 2.77 (br t, 2H), 2.61-2.51 (m, 1H), 2.35- | | | |
| | 2.23 (m, 5H). | | | |
| 456 | 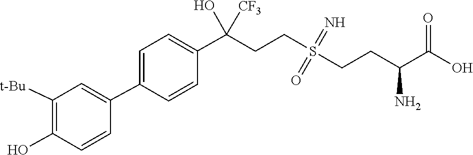 [SMILES: CC(C)(C)c1cc(-c2ccc(C(O)(CCS(=N)(=O)CC[C@H](N)C(=O)O)C(F)(F)F)cc2)ccc1O] | 517.2 | 2.21 | C |
| | ¹H NMR (400 MHz, MeOD-d4) δ 7.67-7.56 (m, | | | |
| | 4H), 7.46 (t, 1H), 7.30 (td, 1H), 6.81 (d, 1H), 3.74- | | | |
| | 3.60 (m, 1H), 3.44-3.35 (m, 1H), 3.28-3.11 (m, | | | |
| | 2H), 2.88-2.70 (m, 2H), 2.66-2.50 (m, 1H), 2.39- | | | |
| | 2.19 (m, 2H), 1.44 (s, 9H) | | | |
| 457 | 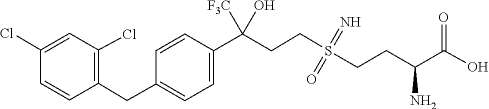 [SMILES: N=S(=O)(CC[C@H](N)C(=O)O)CCC(O)(c1ccc(Cc2ccc(Cl)cc2Cl)cc1)C(F)(F)F] | 527.0 | 2.70 | B |
| | ¹H NMR (400 MHz, MeOD-d₄) δ 7.54 (d, 2H), 7.46 | | | |
| | (d, 1H), 7.29-7.24 (m, 4H), 4.12 (s, 2H), 3.70- | | | |
| | 3.63 (m, 1H), 3.42-3.32 (m, 1H), 3.26-3.13 (m, | | | |
| | 2H), 2.77-2.67 (m, 2H), 2.59-2.50 (m, 1H), 2.31- | | | |
| | 2.23 (m, 2H). | | | |
| 458 | 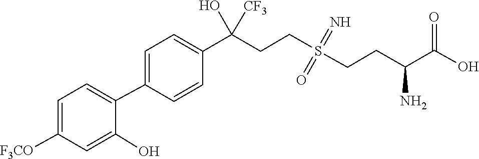 [SMILES: N=S(=O)(CC[C@H](N)C(=O)O)CCC(O)(c1ccc(-c2ccc(OC(F)(F)F)cc2O)cc1)C(F)(F)F] | 545.0 | 2.58 | B |
| | ¹H NMR (400 MHZ, MeOD-d4) δ 7.71-7.62 (m, | | | |
| | 4H), 7.40-7.34 (m, 1H), 6.88-6.80 (m, 2H), 3.75- | | | |
| | 3.64 (m, 1H), 3.28 (br d, 2H), 2.90-2.74 (m, 2H), | | | |
| | 2.68-2.55 (m, 1H), 2.35-2.24 (m, 2H) | | | |

### Exemplary Embodiment 1aa38

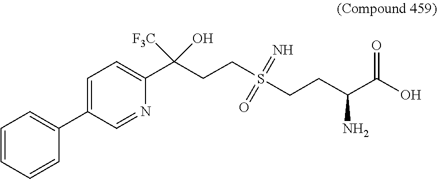 [SMILES: N=S(=O)(CC[C@H](N)C(=O)O)CCC(O)(c1ccc(-c2ccccc2)cn1)C(F)(F)F]

### (2S′)-2-amino-4-(4,4,4-trifluoro-3-hydroxy-3-(5-phenylpyridin-2-yl)butylsulfonimidoyl)butanoic acid

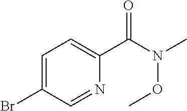

A mixture of 5-bromopyridine-2-carboxylic acid (3.00 g, 14.9 mmol, 1 eq), HATU (7.34 g, 19.3 mmol, 1.3 eq), DIEA (5.76 g, 44.6 mmol, 7.76 mL, 3 eq) and N-methoxymethanamine (1.74 g, 17.8 mmol, 1.2 eq, HCl) in DMF (40 mL) was stirred at 25° C. for 2 h. The mixture was poured into water (100 mL) and extracted with EtOAc (30 mL×3), and the combined organic phases were washed with brine (30 mL), dried over Na₂SO₄, filtered and concentrated. The residue was purified by silica gel chromatography (petroleum ether/EtOAc=10:1 to 5:1) to give 5-bromo-N-methoxy-N-methylpicolinamide (3.4 g, 13.87 mmol, 93.42% yield) as yellow oil. ¹H NMR (400 MHz, CDCl₃-d) δ 8.69 (d, 1H), 7.93 (dd, 1H), 7.73-7.46 (m, 1H), 3.76 (br s, 3H), 3.41 (br s, 3H).

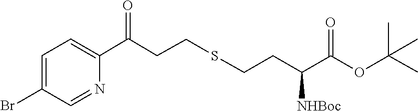 [SMILES: CC(C)(C)OC(=O)N[C@@H](CCSCCC(=O)c1ccc(Br)cn1)C(=O)OC(C)(C)C]

To a solution of 5-bromo-N-methoxy-N-methylpicolinamide (1.00 g, 4.08 mmol, 1 eq) in THE (5 mL) was added bromo(vinyl)magnesium (1 M, 8.16 mL, 2 eq) at −65° C., then tert-butyl 2-(tert-butoxycarbonylamino)-4-sulfanyl-butanoate (595 mg, 2.04 mmol, 0.5 eq) and TEA (1.24 g, 12.2 mmol, 1.70 mL, 3 eq) was added to the mixture at −65° C. and stirred for 1 h. The mixture was quenched with saturated aqueous NH₄Cl (20 mL) and extracted with EtOAc (20 mL×2). The combined organic phases were washed with brine, dried over Na₂SO₄, filtered, and concentrated. The residue was purified by silica gel chromatography (SiO₂, petroleum ether/EtOAc=10:1) and then prep-HPLC (column: Phenomenex C18 (250×70 mm×10 μm); mobile phase: [water(FA)-MeCN]; B %: 55-90%, 20 min) to give tert-butyl S-(3-(5-bromopyridin-2-yl)-3-oxopropyl)-N-(tert-butoxycarbonyl)-L-homocysteinate (1.40 g, 2.78 mmol, 22.72% yield) as yellow oil. ¹H NMR (400 MHz, CDCl₃-d) δ 8.74 (d, 1H), 8.05-7.87 (m, 2H), 5.27-5.01 (m, 1H), 4.37-4.20 (m, 1H), 3.56-3.41 (m, 2H), 2.92 (t, 2H), 2.61 (ddd, 2H), 2.18-2.07 (m, 1H), 1.98-1.86 (m, 1H), 1.49-1.43 (m, 18H).

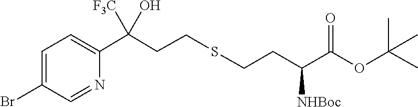 [SMILES: CC(C)(C)OC(=O)N[C@@H](CCSCCC(O)(c1ccc(Br)cn1)C(F)(F)F)C(=O)OC(C)(C)C]

To a solution of tert-butyl S-(3-(5-bromopyridin-2-yl)-3-oxopropyl)-N-(tert-butoxycarbonyl)-L-homocysteinate (800 mg, 1.59 mmol, 1 eq) in THE (20 mL) was added TMSCF₃ (1.13 g, 7.95 mmol, 5 eq), then TBAF (1 M, 159 μL, 0.1 eq) at 0° C. The mixture was stirred for 10 min, then TBAF (1 M, 4.77 mL, 3 eq) was added, and the resulting mixture was stirred at 25° C. for 1 h. The mixture was quenched with water (20 mL) and extracted with EtOAc (20 mL×2). The combined organic phases were washed with brine (20 mL), dried over Na₂SO₄, filtered, and concentrated. The residue was purified by silica gel chromatography (petroleum ether/EtOAc=5:1) to give tert-butyl S-(3-(5-bromopyridin-2-yl)-4,4,4-trifluoro-3-hydroxybutyl)-N-(tert-butoxycarbonyl)-L-homocysteinate (0.45 g, 784.71 μmol, 49.38% yield) as yellow oil. ¹H NMR (400 MHz, CDCl₃-d) δ 8.68 (d, 1H), 7.98 (td, 1H), 7.46 (d, 1H), 5.89 (br d, 1H), 5.19-4.95 (m, 1H), 4.30-4.11 (m, 1H), 2.62-2.44 (m, 4H), 2.37-2.24 (m, 1H), 2.10-1.93 (m, 2H), 1.87-1.74 (m, 1H), 1.46 (d, 18H).

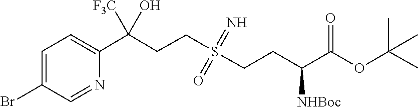 [SMILES: CC(C)(C)OC(=O)N[C@@H](CCS(=N)(=O)CCC(O)(c1ccc(Br)cn1)C(F)(F)F)C(=O)OC(C)(C)C]

A mixture of tert-butyl S-(3-(5-bromopyridin-2-yl)-4,4,4-trifluoro-3-hydroxybutyl)-N-(tert-butoxycarbonyl)-L-homocysteinate (400 mg, 698 μmol, 1 eq), PhI(OAc)₂ (899 mg, 2.79 mmol, 4 eq) and ammonium carbamate (436 mg, 5.58 mmol, 8 eq) in i-PrOH (10 mL) was stirred at 25° C. for 12 h. The mixture was concentrated and the residue was diluted with water (10 mL) and extracted with EtOAc (10 mL×2). The combined organic phases were washed with brine (10 mL), dried over Na₂SO₄, filtered, and concentrated. The residue was purified by silica gel chromatography (petroleum ether/EtOAc=1:1) to give tert-butyl (2S)-4-(3-(5-bromopyridin-2-yl)-4,4,4-trifluoro-3-hydroxybutylsulfonimidoyl)-2-((tert-butoxycarbonyl)amino)butanoate (0.32 g, 529.38 μmol, 75.90% yield) as yellow oil. ¹H NMR (400 MHz, CDCl₃-d) δ 8.69 (d, 1H), 8.09-7.92 (m, 1H), 7.71-7.54 (m, 1H), 5.38-5.16 (m, 1H), 4.34-4.22 (m, 1H), 3.38-3.00 (m, 3H), 2.95-2.60 (m, 3H), 2.46-2.29 (m, 1H), 2.15-2.06 (m, 1H), 1.49 (s, 3H), 1.43 (s, 9H).

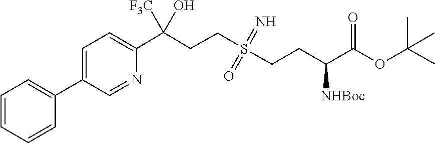 [SMILES: CC(C)(C)OC(=O)N[C@@H](CCS(=N)(=O)CCC(O)(c1ccc(-c2ccccc2)cn1)C(F)(F)F)C(=O)OC(C)(C)C]

A mixture of give tert-butyl (2S)-4-(3-(5-bromopyridin-2-yl)-4,4,4-trifluoro-3-hydroxybutylsulfonimidoyl)-2-((tert-butoxycarbonyl)amino)butanoate (100 mg, 165 μmol, 1 eq), phenylboronic acid (30.3 mg, 248 μmol, 1.5 eq), Na₂CO₃ (43.8 mg, 414 μmol, 2.5 eq) and Pd(dppf)Cl₂ (13.5 mg, 16.5 μmol, 0.1 eq) in t-BuOH (2.4 mL)/H₂O (0.8 mL) was de-gassed and then heated to 90° C. for 1.5 h under N₂. The mixture was diluted with water (10 mL) and extracted with EtOAc (10 mL×2). The combined organic phases were washed with brine (10 mL), dried over Na₂SO₄, filtered, and concentrated. The residue was purified by prep-TLC (petroleum ether/EtOAc=1:1+0.1% TEA) to give tert-butyl (2S)-2-((tert-butoxycarbonyl)amino)-4-(4,4,4-trifluoro-3-hydroxy-3-(5-phenylpyridin-2-yl)butylsulfonimidoyl)butanoate (80 mg, 132.96 μmol, 80.37% yield) as yellow oil.

A mixture of tert-butyl (2S)-2-((tert-butoxycarbonyl)amino)-4-(4,4,4-trifluoro-3-hydroxy-3-(5-phenylpyridin-2-yl)butylsulfonimidoyl)butanoate (80 mg, 133 μmol, 1 eq) in HCl/dioxane (10 mL) was stirred at 25° C. for 6 h. The mixture was concentrated and the residue was purified by prep-HPLC (column: Phenomenex C18 (75×30 mm, 3 μm); mobile phase: [water (FA)-MeCN]; gradient: 20-50% B over 10 min) to give (2S)-2-amino-4-(4,4,4-trifluoro-3-hydroxy-3-(5-phenylpyridin-2-yl)butylsulfonimidoyl)butanoic acid (25 mg, 51.28 μmol, 38.57% yield, 91.38% purity) as white solid. LCMS: Rt=1.944 min, (ES⁺) m/z (M+H)⁺=446.1, HPLC Conditions: C. ¹H NMR (400 MHz, MeOD-d₄) δ 9.02-8.83 (m, 1H), 8.27-8.08 (m, 1H), 7.90 (d, 1H), 7.73 (d, 2H), 7.57-7.50 (m, 2H), 7.50-7.43 (m, 1H), 3.78-3.65 (m, 1H), 3.47-3.35 (m, 1H), 3.28-3.17 (m, 1H), 3.01 (dt, 1H), 2.89-2.76 (m, 1H), 2.69-2.56 (m, 1H), 2.33 (dt, 2H).

### Exemplary Embodiment 1aa39

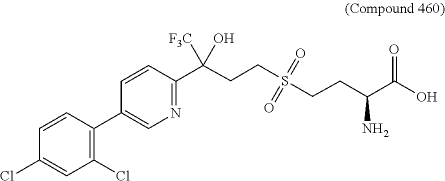 [SMILES: N[C@@H](CCS(=O)(=O)CCC(O)(c1ccc(-c2ccc(Cl)cc2Cl)cn1)C(F)(F)F)C(=O)O]

### (2S)-2-amino-4-[3-[5-(2,4-dichlorophenyl)-2-pyridyl]-4,4,4-trifluoro-3-hydroxy-butyl]sulfonyl-butanoic acid

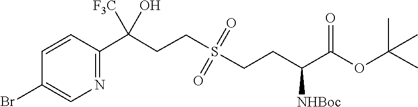 [SMILES: CC(C)(C)OC(=O)N[C@@H](CCS(=O)(=O)CCC(O)(c1ccc(Br)cn1)C(F)(F)F)C(=O)OC(C)(C)C]

To a solution of tert-butyl (2S)-4-[3-(5-bromo-2-pyridyl)-4,4,4-trifluoro-3-hydroxy-butyl]sulfanyl-2-(tert-butoxycarbonylamino)butanoate (250 mg, 435 μmol, 1 eq) in DCM (5 mL) was added m-CPBA (188 mg, 871 μmol, 80% purity, 2 eq). The mixture was stirred at 20° C. for 1 h. The mixture was poured into NaHCO₃ (10 mL) and extracted with DCM (10 mL×2). The combined organic layers were dried over Na₂SO₄, filtered, and concentrated under reduced pressure. The residue was purified by prep-TLC (SiO₂, petroleum ether/EtOAc=2:1, R_{f}=0.43) to give tert-butyl (2S)-4-[3-(5-bromo-2-pyridyl)-4,4,4-trifluoro-3-hydroxy-butyl]sulfonyl-2-(tert-butoxycarbonylamino)butanoate (173 mg, 285 μmol, 65.5% yield) as a yellow oil. ¹H NMR (400 MHz, CDCl₃-d) δ 8.71 (d, 1H), 8.02 (td, 1H), 7.55-7.46 (m, 1H), 5.26-5.08 (m, 1H), 4.30-4.16 (m, 1H), 3.24-2.89 (m, 3H), 2.78-2.64 (m, 1H), 2.63-2.49 (m, 2H), 2.42-2.29 (m, 1H), 2.07-2.05 (m, 1H), 2.14-1.99 (m, 1H), 1.50-1.44 (m, 18H).

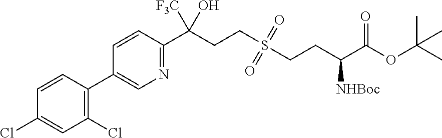 [SMILES: CC(C)(C)OC(=O)N[C@@H](CCS(=O)(=O)CCC(O)(c1ccc(-c2ccc(Cl)cc2Cl)cn1)C(F)(F)F)C(=O)OC(C)(C)C]

To a solution of tert-butyl (2S)-4-[3-(5-bromo-2-pyridyl)-4,4,4-trifluoro-3-hydroxy-butyl]sulfonyl-2-(tert-butoxycarbonylamino)butanoate (100 mg, 165 μmol, 1 eq) and (2,4-dichlorophenyl)boronic acid (37.8 mg, 198 μmol, 1.2 eq) in t-BuOH (3 mL) and H₂O (1 mL) was added Na₂CO₃ (52.5 mg, 495 μmol, 3 eq) and Pd(dppf)Cl₂ (12.0 mg, 16.5 μmol, 0.1 eq). The mixture was stirred at 100° C. for 2 h under N₂. The mixture was poured into water (10 mL) and extracted with EtOAc (5 mL×3). The combined organic layers were dried over Na₂SO₄, filtered, and concentrated under reduced pressure. The residue was purified by prep-TLC (SiO₂, petroleum ether/EtOAc=2:1, R_{f}=0.45) to give tert-butyl (2S)-2-(tert-butoxycarbonylamino)-4-[3-[5-(2,4-dichlorophenyl)-2-pyridyl]-4,4,4-trifluoro-3-hydroxy-butyl]sulfonyl-butanoate (107 mg, 159. μmol, 96.4% yield) as a yellow oil. ¹H NMR (400 MHz, CDCl₃-d) δ 8.72-8.66 (m, 1H), 8.05-7.94 (m, 1H), 7.76-7.63 (m, 1H), 7.59-7.48 (m, 1H), 7.43-7.28 (m, 2H), 5.24-5.12 (m, 1H), 4.35-4.21 (m, 1H), 3.28-2.94 (m, 3H), 2.82-2.51 (m, 3H), 2.43-2.31 (m, 1H), 2.15-2.00 (m, 1H), 1.47 (br d, 18H)

A solution of tert-butyl (2S)-2-(tert-butoxycarbonylamino)-4-[3-[5-(2,4-dichlorophenyl)-2-pyridyl]-4,4,4-trifluoro-3-hydroxy-butyl]sulfonyl-butanoate (105 mg, 156 μmol, 1 eq) in HCl/dioxane (6 M, 30.0 mL, 1151 eq) was stirred at 25° C. for 16 h. The reaction mixture was concentrated under reduced pressure. The residue was purified by prep-HPLC (column: Phenomenex Luna C18 (200×mm, 10 μm); mobile phase: [H₂O (0.2% FA)-MeCN]; gradient: 20-55% B over 8.0 min) to give (2S)-2-amino-4-[3-[5-(2,4-dichlorophenyl)-2-pyridyl]-4,4,4-trifluoro-3-hydroxy-butyl]sulfonyl-butanoic acid (31.2 mg, 60.6 μmol, 38.8% yield) as a white solid. LCMS: Rt=2.351 min, (ES⁺) m/z (M+H)⁺=515.0, HPLC Conditions: C. ¹H NMR (400 MHz, MeOD-d₄) δ 8.74-8.67 (m, 1H), 8.02 (dd, 1H), 7.90 (d, 1H), 7.65 (s, 1H), 7.47 (s, 2H), 3.69 (dt, 1H), 3.42-3.33 (m, 1H), 3.27-3.10 (m, 2H), 2.99 (dt, 1H), 2.82-2.71 (m, 1H), 2.64-2.51 (m, 1H), 2.33-2.22 (m, 2H).

### Exemplary Embodiment 1aa40

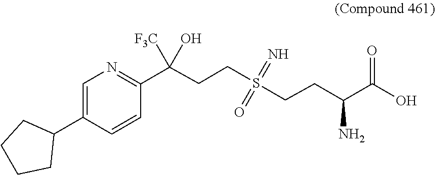 [SMILES: N=S(=O)(CC[C@H](N)C(=O)O)CCC(O)(c1ccc(C2CCCC2)cn1)C(F)(F)F]

### (2S)-2-amino-4-(3-(5-cyclopentylpyridin-2-yl)-4,4,4-trifluoro-3-hydroxybutylsulfonimidoyl)butanoic acid

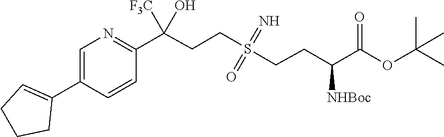 [SMILES: CC(C)(C)OC(=O)N[C@@H](CCS(=N)(=O)CCC(O)(c1ccc(C2=CCCC2)cn1)C(F)(F)F)C(=O)OC(C)(C)C]

A mixture of tert-butyl (2S)-4-(3-(5-bromopyridin-2-yl)-4,4,4-trifluoro-3-hydroxybutylsulfonimidoyl)-2-((tert-butoxycarbonyl)amino)butanoate (155 mg, 256 μmol, 1 eq) and 2-(cyclopenten-1-yl)-4,4,5,5-tetramethyl-1,3,2-dioxaborolane (74.6 mg, 384 μmol, 1.5 eq) in dioxane (2.5 mL) and H₂O (0.5 mL) was added Na₂CO₃ (81.5 mg, 769 μmol, 3 eq) and Pd(PPh₃)₄, (29.6 mg, 25.6 μmol, 0.1 eq) under N₂. The mixture was stirred at 90° C. for 16 h under N₂. The mixture was poured into water (5 mL) and extracted with EtOAc (10 mL×3). The combined organic layers were dried over Na₂SO₄, filtered, and concentrated under reduced pressure. The residue was purified by prep-HPLC (column: Phenomenex Luna C18 (200×mm, 10 μm); mobile phase: [H₂O (0.2% FA)-MeCN]; gradient: 45-75% B over 8.0 min) to give tert-butyl (2S)-2-((tert-butoxycarbonyl)amino)-4-(3-(5-(cyclopent-1-en-1-yl)pyridin-2-yl)-4,4,4-trifluoro-3-hydroxybutylsulfonimidoyl)butanoate (40 mg, 67.60 μmol) as a yellow oil.

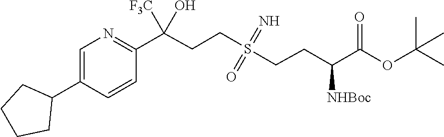 [SMILES: CC(C)(C)OC(=O)N[C@@H](CCS(=N)(=O)CCC(O)(c1ccc(C2CCCC2)cn1)C(F)(F)F)C(=O)OC(C)(C)C]

To a solution of tert-butyl (2S)-2-((tert-butoxycarbonyl)amino)-4-(3-(5-(cyclopent-1-en-1-yl)pyridin-2-yl)-4,4,4-trifluoro-3-hydroxybutylsulfonimidoyl)butanoate (30 mg, 50.7 μmol, 1 eq) in EtOH (1 mL) was added Pd/C (30 mg, 28.1 μmol, 10% purity, 0.55 eq). The mixture was stirred at 25° C. for 2 h under H₂ (15 psi). The mixture was filtered and concentrated under reduced pressure to give tert-butyl (2S)-2-((tert-butoxycarbonyl)amino)-4-(3-(5-cyclopentylpyridin-2-yl)-4,4,4-trifluoro-3-hydroxybutylsulfonimidoyl)butanoate (27 mg, 45.4 μmol, 89.6% yield) as a yellow oil.

A solution of tert-butyl (2S)-2-((tert-butoxycarbonyl)amino)-4-(3-(5-cyclopentylpyridin-2-yl)-4,4,4-trifluoro-3-hydroxybutylsulfonimidoyl)butanoate (27 mg, 45.4 μmol, 1 eq) in HCl/dioxane (4 M, 11.3 μL, 1 eq) was stirred at 30° C. for 6 h. The reaction mixture was concentrated under reduced pressure and the residue was purified by prep-HPLC (column: Phenomenex Luna C18 (200×40 mm, 10 μm); mobile phase: [H₂O (0.2% FA)-MeCN]; gradient: 30-60% B over 8.0 min) to give (2S)-2-amino-4-(3-(5-cyclopentylpyridin-2-yl)-4,4,4-trifluoro-3-hydroxybutylsulfonimidoyl)butanoic acid (10 mg, 22.8 μmol, 50.2% yield) as a white solid. LCMS: Rt=2.056 min, (ES⁺) m/z (M+H)⁺=438.1, HPLC Conditions: C. ¹H NMR (400 MHz, MeOD-d₄) δ 8.53 (d, 1H), 7.82 (dd, 1H), 7.71 (d, 1H), 3.74-3.61 (m, 1H), 3.44-3.33 (m, 1H), 3.28-3.04 (m, 3H), 2.97-2.84 (m, 1H), 2.81-2.67 (m, 1H), 2.63-2.49 (m, 1H), 2.40-2.22 (m, 2H), 2.20-2.07 (m, 2H), 1.96-1.71 (m, 4H), 1.70-1.55 (m, 2H).

The compounds described in Table 19 were prepared using the general methods outlined above.

TABLE 19
Characterization of Compounds 462-481

| Compound | | MS | HPLC | HPLC |
|---|---|---|---|---|
| # | Structure | (M + H)⁺ | Rt | cond. |
| 462 | 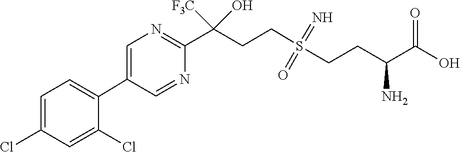 [SMILES: N=S(=O)(CC[C@H](N)C(=O)O)CCC(O)(c1ncc(-c2ccc(Cl)cc2Cl)cn1)C(F)(F)F] | 514.1 | 2.17 | C |
| | (FA salt) | | | |
| | ¹H NMR (400 MHz, MeOD-d₄) δ 8.71 (d, 1H), 8.05- | | | |
| | 8.00 (m, 1H), 7.91 (d, 1H), 7.65 (d, 1H), 7.49- | | | |
| | 7.47 (m, 1H), 7.51-7.45 (m, 1H), 3.73-3.66 (m, | | | |
| | 1H), 3.73-3.65 (m, 1H), 3.44-3.35 (m, 1H), 3.30- | | | |
| | 3.18 (m, 2H), 3.06-2.98 (m, 1H), 2.90-2.76 (m, | | | |
| | 1H), 2.67-2.55 (m, 1H), 2.43-2.20 (m, 2H). | | | |
| 463 | 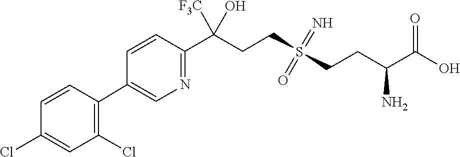 | 513.9 | 2.572 | B |
| | (Isomer 1) | | | |
| | ¹H NMR (400 MHz, DMSO-d₆) δ 8.72 (d, 1H), | | | |
| | 8.04 (dd, 1H), 7.88 (d, 1H), 7.82 (d, 1H), 7.60- | | | |
| | 7.57 (m, 2H), 3.38-3.34 (m, 2H), 3.18-3.03 (m, | | | |
| | 3H), 2.93-2.79 (m, 1H), 2.43 (br d, 1H), 2.09- | | | |
| | 2.00 (m, 2H) | | | |
| 464 | 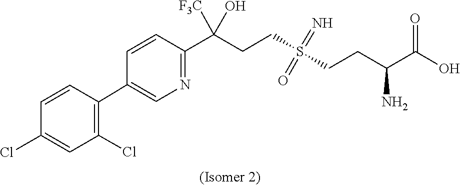 | 513.9 | 2.572 | B |
| | ¹H NMR (400 MHz, DMSO-d₆) δ 8.71 (d, 1H), | | | |
| | 8.04 (dd, 1H), 7.88 (d, 1H), 7.82 (d, 1H), 7.60- | | | |
| | 7.57 (m, 2H), 3.30 (br d, 2H), 3.12-2.98 (m, 2H), | | | |
| | 2.85 (dt, 1H), 2.63-2.53 (m, 1H), 2.48-2.42 (m, | | | |
| | 1H), 2.08-1.95 (m, 2H) | | | |
| 465 | 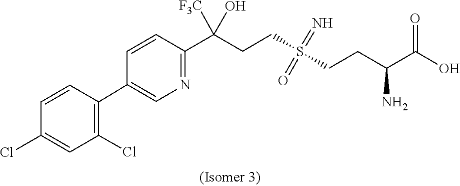 | 513.9 | 2.576 | B |
| | ¹H NMR (400 MHz, DMSO-d₆) δ 8.71 (d, 1H), | | | |
| | 8.03 (dd, 1H), 7.88 (d, H), 7.81 (d, 1H), 7.60-7.56 | | | |
| | (m, 2H), 3.25-3.03 (m, 4H), 2.86 (dt, 1H), 2.44 (br | | | |
| | d, 2H), 2.11-1.99 (m, 2H) | | | |
| 466 | 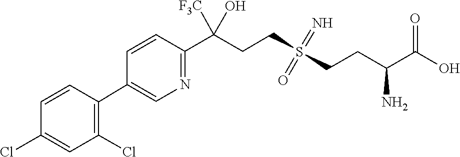 | 513.9 | 2.570 | B |
| | (Isomer 4) | | | |
| | ¹H NMR (400 MHz, DMSO-d₆) δ 8.74-8.67 (m, | | | |
| | 1H), 8.04 (dd, 1H), 7.88 (d, 1H), 7.82 (s, 1H), 7.59 | | | |
| | (br d, 2H), 3.31-3.25 (m, 2H), 3.12-2.98 (m, 2H), | | | |
| | 2.90-2.78 (m, 1H), 2.56 (br dd, 2H), 2.08-1.92 | | | |
| | (m, 2H) | | | |
| 467 | 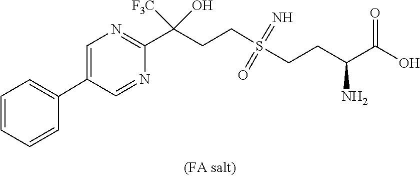 [SMILES: N=S(=O)(CC[C@H](N)C(=O)O)CCC(O)(c1ncc(-c2ccccc2)cn1)C(F)(F)F] | 447.1 | 1.83 | C |
| | ¹H NMR (400 MHz, MeOD-d₄) δ 9.20 (s, 2H), 7.89- | | | |
| | 7.70 (m, 2H), 7.65-7.43 (m, 3H), 3.81-3.69 (m, | | | |
| | 1H), 3.49-3.37 (m, 1H), 3.33-3.23 (m, 2H), 3.05 | | | |
| | (dt, 1H), 2.97-2.84 (m, 1H), 2.74-2.58 (m, 1H), | | | |
| | 2.35 (dt, 2H). | | | |
| 468 | 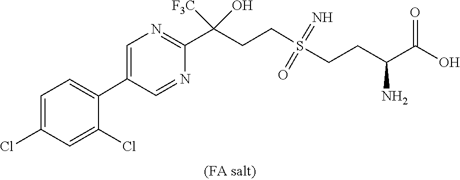 [SMILES: N=S(=O)(CC[C@H](N)C(=O)O)CCC(O)(c1ncc(-c2ccc(Cl)cc2Cl)cn1)C(F)(F)F] | 515.1 | 2.06 | C |
| | ¹H NMR (400 MHz, MeOD-d₄) δ 9.07 (s, 2H), 8.21 | | | |
| | (s, 1H), 7.73 (d, 1H), 7.62-7.52 (m, 2H), 3.79- | | | |
| | 3.69 (m, 1H), 3.53-3.34 (m, 2H), 3.31-3.25 (m, | | | |
| | 1H), 3.11-3.00 (m, 1H), 2.99-2.87 (m, 1H), 2.75- | | | |
| | 2.60 (m, 1H), 2.45-2.26 (m, 2H). | | | |
| 469 | 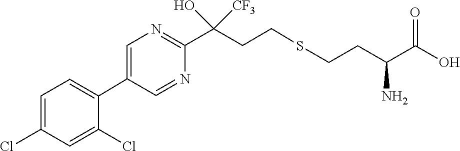 [SMILES: N[C@@H](CCSCCC(O)(c1ncc(-c2ccc(Cl)cc2Cl)cn1)C(F)(F)F)C(=O)O] | 484.1 | 2.195 | C |
| | ¹H NMR (400 MHz, MeOD-d₄) δ = 9.02 (s, 2H), | | | |
| | 7.74-7.68 (m, 1H), 7.60-7.48 (m, 2H), 3.66-3.56 | | | |
| | (m, 1H), 2.89-2.77 (m, 1H), 2.70-2.54 (m, 3H), | | | |
| | 2.43-2.32 (m, 1H), 2.30-2.18 (m, 1H), 2.15-2.05 | | | |
| | (m, 1H), 2.01-1.91 (m, 1H) | | | |
| 470 | 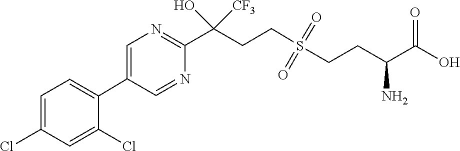 [SMILES: N[C@@H](CCS(=O)(=O)CCC(O)(c1ncc(-c2ccc(Cl)cc2Cl)cn1)C(F)(F)F)C(=O)O] | 516.1 | 2.17 | C |
| | ¹H NMR (400 MHz, MeOD-d₄) δ 9.04 (s, 2H), 7.70 | | | |
| | (d, 1H), 7.60-7.48 (m, 2H), 3.68-3.57 (m, 1H), | | | |
| | 3.32 (br s, 1H), 3.30-3.19 (m, 2H), 3.01 (dt, 1H), | | | |
| | 2.93-2.81 (m, 1H), 2.68-2.55 (m, 1H), 2.33-2.16 | | | |
| | (m, 2H) | | | |
| 471 | 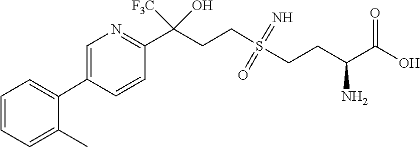 [SMILES: Cc1ccccc1-c1ccc(C(O)(CCS(=N)(=O)CC[C@H](N)C(=O)O)C(F)(F)F)nc1] | 460.1 | 2.08 | C |
| | ¹H NMR (400 MHz, MeOD-d₄) δ 8.61 (s, 1H), 7.97- | | | |
| | 7.81 (m, 2H), 7.38-7.19 (m, 4H), 3.75-3.62 (m, | | | |
| | 1H), 3.46-3.35 (m, 1H), 3.29-3.15 (m, 2H), 3.06- | | | |
| | 2.94 (m, 1H), 2.90-2.77 (m, 1H), 2.67-2.53 (m, | | | |
| | 1H), 2.39-2.25 (m, 5H) | | | |
| 472 | 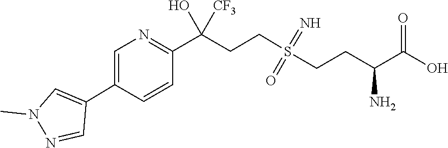 [SMILES: Cn1cc(-c2ccc(C(O)(CCS(=N)(=O)CC[C@H](N)C(=O)O)C(F)(F)F)nc2)cn1] | 450.1 | 1.67 | C |
| | ¹H NMR (400 MHz, D₂O) δ 8.69 (s, 1H), 8.00 (br | | | |
| | s, 2H), 7.89 (s, 1H), 7.72 (br d, 1H), 3.88 (s, 3H), | | | |
| | 3.82 (br d, 1H), 3.38 (ddd, 1H), 3.34-3.22 (m, 2H), | | | |
| | 2.92-2.79 (m, 2H), 2.65-2.52 (m, 1H), 2.31-2.21 | | | |
| | (m, 2H) | | | |
| 473 | 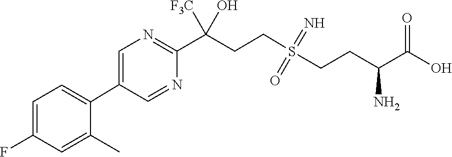 [SMILES: Cc1cc(F)ccc1-c1cnc(C(O)(CCS(=N)(=O)CC[C@H](N)C(=O)O)C(F)(F)F)nc1] | 479.1 | 2.02 | C |
| | ¹H NMR (400 MHz, MeOD-d4) δ 8.93 (s, 2H), | | | |
| | 7.36 (dd, 1H), 7.20-7.04 (m, 2H), 3.77-3.65 (m, | | | |
| | 1H), 3.51-3.32 (m, 2H), 3.29-3.17 (m, 1H), 3.10- | | | |
| | 2.86 (m, 2H), 2.73-2.57 (m, 1H), 2.43-2.24 (m, | | | |
| | 5H) | | | |
| 474 | 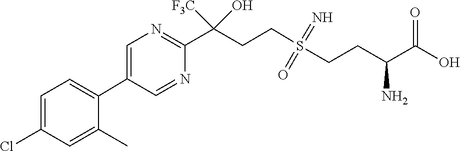 [SMILES: Cc1cc(Cl)ccc1-c1cnc(C(O)(CCS(=N)(=O)CC[C@H](N)C(=O)O)C(F)(F)F)nc1] | 495.1 | 2.13 | C |
| | ¹H NMR (400 MHz, MeOD-d₄) δ 8.94 (s, 2H), 7.43 | | | |
| | (s, 1H), 7.39-7.31 (m, 2H), 3.74-3.64 (m, 1H), | | | |
| | 3.44-3.33 (m, 2H), 3.28-3.21 (m, 1H), 3.09-2.88 | | | |
| | (m, 2H), 2.72-2.59 (m, 1H), 2.38-2.26 (m, 5H) | | | |
| 475 | 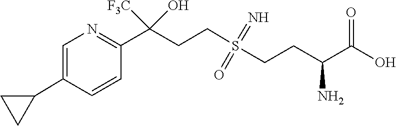 [SMILES: N=S(=O)(CC[C@H](N)C(=O)O)CCC(O)(c1ccc(C2CC2)cn1)C(F)(F)F] | 410.1 | 1.81 | C |
| | ¹H NMR (400 MHz, MeOD-d₄) δ 8.44 (s, 1H), 7.66 | | | |
| | (d, 1H), 7.54 (dd, 1H), 3.76-3.38 (m, 2H), 3.28- | | | |
| | 3.10 (m, 2H), 2.99-2.79 (m, 1H), 2.76-2.60 (m, | | | |
| | 1H), 2.60-2.49 (m, 1H), 2.29 (br dd, 2H), 2.04- | | | |
| | 1.96 (m, 1H), 1.14-1.04 (m, 2H), 0.85-0.75 (m, | | | |
| | 2H) | | | |
| 476 | 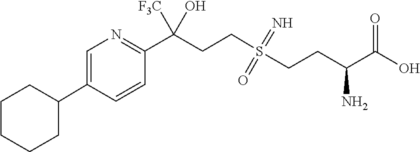 [SMILES: N=S(=O)(CC[C@H](N)C(=O)O)CCC(O)(c1ccc(C2CCCCC2)cn1)C(F)(F)F] | 452.2 | 2.05 | C |
| | ¹H NMR (400 MHz, MeOD-d₄) δ 8.46 (s, 1H), 7.88- | | | |
| | 7.83 (m, 1H), 7.70 (d, 1H), 3.88-3.78 (m, 1H), | | | |
| | 3.44-3.21 (m, 4H), 2.93-2.78 (m, 2H), 2.71-2.53 | | | |
| | (m, 2H), 2.33-2.23 (m, 2H), 1.90-1.65 (m, 6H), | | | |
| | 1.48-1.16 (m, 6H) | | | |
| 477 | 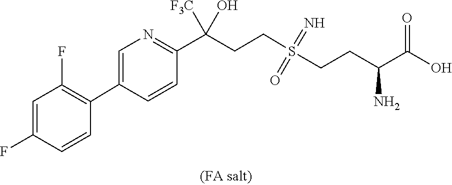 [SMILES: N=S(=O)(CC[C@H](N)C(=O)O)CCC(O)(c1ccc(-c2ccc(F)cc2F)cn1)C(F)(F)F] | 482.1 | 2.66 | D |
| | ¹H NMR (400 MHz, MeOD-d₄) δ 8.80 (s, 1H), 8.08 | | | |
| | (br d, 1H), 7.90 (d, 1H), 7.67-7.59 (m, 1H), 7.19- | | | |
| | 7.09 (m, 2H), 3.73-3.63 (m, 1H), 3.49 (s, 1H), | | | |
| | 3.25-3.10 (m, 2H), 3.06-2.95 (m, 1H), 2.88-2.74 | | | |
| | (m, 1H), 2.66-2.51 (m, 1H), 2.38-2.23 (m, 2H) | | | |
| 478 | 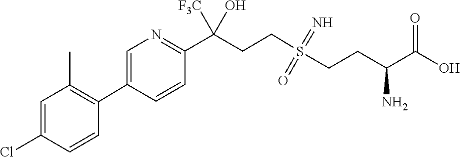 [SMILES: Cc1cc(Cl)ccc1-c1ccc(C(O)(CCS(=N)(=O)CC[C@H](N)C(=O)O)C(F)(F)F)nc1] | 494.0 | 2.22 | C |
| | ¹H NMR (400 MHz, MeOD-d₄) δ 8.61 (s, 1H), 7.96- | | | |
| | 7.85 (m, 2H), 7.41-7.23 (m, 3H), 3.73-3.65 (m, | | | |
| | 1H), 3.42-3.33 (m, 2H), 3.26-3.18 (m, 1H), 3.06- | | | |
| | 2.94 (m, 1H), 2.91-2.77 (m, 1H), 2.67-2.54 (m, | | | |
| | 1H), 2.35-2.26 (m, 5H) | | | |
| 479 | 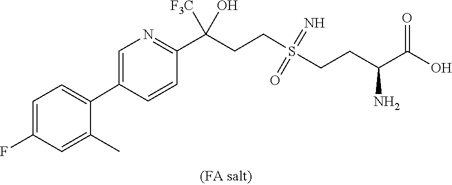 [SMILES: Cc1cc(F)ccc1-c1ccc(C(O)(CCS(=N)(=O)CC[C@H](N)C(=O)O)C(F)(F)F)nc1] | 478.1 | 2.12 | C |
| | ¹H NMR (400 MHz, MeOD-d₄) δ 8.60 (s, 1H), 8.19 | | | |
| | (s, 1H), 7.91-7.86 (m, 2H), 7.32-7.24 (m, 1H), | | | |
| | 7.12-7.01 (m, 2H), 3.75-3.66 (m, 1H), 3.44-3.35 | | | |
| | (m, 1H), 3.29-3.16 (m, 2H), 3.05-2.95 (m, 1H), | | | |
| | 2.91-2.78 (m, 1H), 2.68-2.55 (m, 1H), 2.36-2.27 | | | |
| | (m, 5H) | | | |
| 480 | 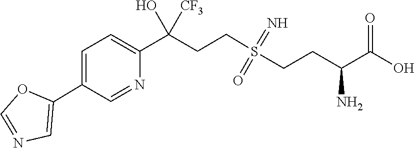 [SMILES: N=S(=O)(CC[C@H](N)C(=O)O)CCC(O)(c1ccc(-c2cnco2)cn1)C(F)(F)F] | 437.0 | 1.83 | B |
| | ¹H NMR (400 MHz, MeOD-d₄) δ 9.02 (s, 1H), 8.36 | | | |
| | (s, 1H), 8.24 (dd, 1H), 7.91 (d, 1H), 7.75 (s, 1H), | | | |
| | 3.73-3.63 (m, 1H), 3.45-3.34 (m, 1H), 3.29-3.11 | | | |
| | (m, 2H), 3.01 (dt, 1H), 2.86-2.74 (m, 1H), 2.64- | | | |
| | 2.52 (m, 1H), 2.30 (br d, 2H) | | | |
| 481 | 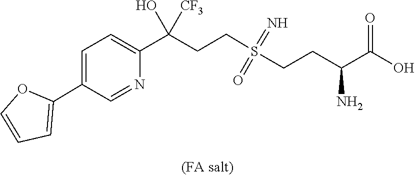 [SMILES: N=S(=O)(CC[C@H](N)C(=O)O)CCC(O)(c1ccc(-c2ccco2)cn1)C(F)(F)F] | 436.1 | 1.84 | C |
| | ¹H NMR (400 MHz, D₂O) δ 8.90 (d, 1H), 8.41 (s, | | | |
| | 1H), 7.81 (d, 1H), 7.66 (s, 1H), 7.00 (d, 1H), 6.61 | | | |
| | (dd, 1H), 3.87-3.79 (m, 1H), 3.46-3.21 (m, 3H), | | | |
| | 3.01-2.78 (m, 2H), 2.68-2.52 (m, 1H), 2.38-2.18 | | | |
| | (m, 2H) | | | |

### Exemplary Embodiment 1aa40.2

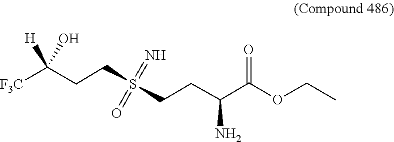 [SMILES: CCOC(=O)[C@@H](N)CC[S@@](=N)(=O)CC[C@@H](O)C(F)(F)F]

### ethyl (S)-2-amino-4-((R,3R)-4,4,4-trifluoro-3-hydroxybutylsulfonimidoyl)butanoate

To a solution of (S)-2-amino-4-((R,3R)-4,4,4-trifluoro-3-hydroxybutylsulfonimidoyl)butanoic acid (Compound 328 (see also Exemplary Embodiment 1aa18.3), 100 mg, 342 μmol, 1 eq) in EtOH (3 mL) was added SOCl₂ (407 mg, 3.42 mmol, 248 μL, 10 eq). The mixture was stirred at 30° C. for 12 h under N₂. The reaction mixture was concentrated and the residue was purified by prep-HPLC (column: Waters Xbridge BEH C18 (100×30 mm, 10 μm); mobile phase: [H₂O (10 mM NH₄HCO₃)-MeCN]; gradient: 1-30% B, 8.0 min) to give ethyl (S)-2-amino-4-((R,3R)-4,4,4-trifluoro-3-hydroxybutylsulfonimidoyl)butanoate (43 mg, 134 μmol, 39.2% yield) as a white solid. LCMS: Rt=2.040 min, (ES⁺) m/z (M+H)⁺=321.0, HPLC Conditions: I. ¹H NMR (400 MHz, D₂O) δ 4.27-4.16 (m, 3H), 3.68 (br t, 1H), 3.44-3.28 (m, 4H), 2.31-2.17 (m, 2H), 2.15-2.02 (m, 2H), 1.27-1.21 (m, 3H).

### Exemplary Embodiment 1aa40.3

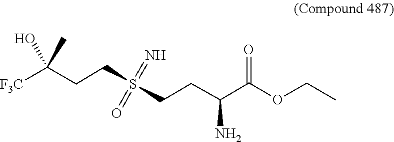 [SMILES: CCOC(=O)[C@@H](N)CC[S@@](=N)(=O)CC[C@@](C)(O)C(F)(F)F]

### ethyl (S)-2-amino-4-((R,3R)-4,4,4-trifluoro-3-hydroxy-3-methylbutylsulfonimidoyl)butanoate

To a solution of (S)-2-amino-4-((R,3R)-4,4,4-trifluoro-3-hydroxy-3-methylbutylsulfonimidoyl)butanoic acid (Compound 322 (see also Exemplary Embodiment 1aa18.2), 150 mg, 489 μmol, 1 eq) in EtOH (3 mL) was added SOCl₂ (583 mg, 4.90 mmol, 356 μL, 10 eq) at 30° C. The resulting mixture was stirred at 30° C. for 12 h. The reaction mixture was concentrated under reduced pressure. The residue was purified by prep-HPLC (column: Phenomenex Gemini-NX C18 (75×30 mm, 3 μm; mobile phase: [H₂O (10 mM NH₄HCO₃)-MeCN]; gradient: 10-50% B, 9.0 min) to give ethyl (S)-2-amino-4-((R,3R)-4,4,4-trifluoro-3-hydroxy-3-methylbutylsulfonimidoyl)butanoate (126 mg, 370 μmol, 75.6% yield) as a colorless oil. LCMS: Rt=1.647 min, (ES⁺) m/z (M+H)⁺=335.1, HPLC Conditions: D. 1H NMR (400 MHz, D₂O) δ 4.20 (q, 2H), 3.72 (t, 1H), 3.45-3.27 (m, 4H), 2.30-2.06 (m, 4H), 1.37 (s, 3H), 1.24 (t, 3H).

### Exemplary Embodiment 1aa40.4

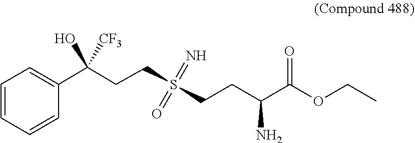 [SMILES: CCOC(=O)[C@@H](N)CC[S@@](=N)(=O)CC[C@](O)(c1ccccc1)C(F)(F)F]

### ethyl (S)-2-amino-4-((R,3S)-4,4,4-trifluoro-3-hydroxy-3-phenylbutylsulfonimidoyl)butanoate

To a solution of (S)-2-amino-4-((R,3S)-4,4,4-trifluoro-3-hydroxy-3-phenylbutylsulfonimidoyl)butanoic acid (Compound 348 (see also Exemplary Embodiment 1aa21.2), 130 mg, 352 μmol, 1 eq) in EtOH (3 mL) was added SOCl₂ (419 mg, 3.53 mmol, 256 μL, 10 eq). The mixture was stirred at 20° C. for 16 h. The reaction mixture was concentrated and the residue was purified by prep-HPLC (column: Waters Xbridge BEH C18 (100×30 mm, 10 μm); mobile phase: [H₂O (10 mM NH₄HCO₃)-(1:3 THF/MeCN)]; gradient:1-35% B, 15.0 min) to give ethyl (S)-2-amino-4-((R,3S)-4,4,4-trifluoro-3-hydroxy-3-phenylbutylsulfonimidoyl)butanoate (28.2 mg, 70.4 μmol, 19.9% yield, 98.9% purity) as a white solid. LCMS: Rt=3.215 min, (ES⁺) m/z (M+H)⁺=397.0, HPLC Conditions: I. ¹H NMR (400 MHz, D₂O) δ 7.61 (br d, 2H), 7.54-7.46 (m, 3H), 4.21-4.13 (m, 2H), 3.64-3.57 (m, 1H), 3.32-3.16 (m, 3H), 2.94-2.82 (m, 1H), 2.77-2.67 (m, 1H), 2.65-2.54 (m, 1H), 2.19-2.04 (m, 1H), 2.00-1.87 (m, 1H), 1.22 (t, 3H).

### Exemplary Embodiment 1aa40.5

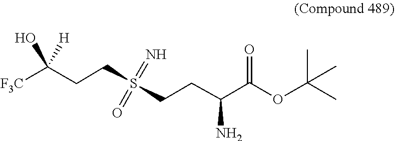 [SMILES: CC(C)(C)OC(=O)[C@@H](N)CC[S@@](=N)(=O)CC[C@H](O)C(F)(F)F]

### tert-butyl (S)-2-amino-4-((R,3S)-4,4,4-trifluoro-3-hydroxybutylsulfonimidoyl)butanoate

To a mixture of (S)-2-amino-4-((R,3S)-4,4,4-trifluoro-3-hydroxybutylsulfonimidoyl)butanoic acid (Compound 326 (see also Exemplary Embodiment 1aa18.3), 300 mg, 1.03 mmol) in tert-butyl acetate (10 mL) was added HClO₄ (589 mg, 4.11 mmol, 354 μL, 70% purity, 4 eq) in one portion at 0° C. The mixture was stirred at 25° C. for 12 h. The mixture was cooled to 0° C. and adjusted to pH=8 with 2M aqueous NaOH solution. The organic phase was separated and concentrated under vacuum. The residue was purified by prep-HPLC (column: Phenomenex C18 (75×30 mm, 3 μm); mobile phase: [water (FA)-MeCN]; gradient: 10-40% B, 10 min) to afford tert-butyl (S)-2-amino-4-((R,3S)-4,4,4-trifluoro-3-hydroxybutylsulfonimidoyl)butanoate (98.7 mg, 283 μmol, 27.6% yield) as white solid. LCMS: Rt=1.625 min, (ES⁺) m/z (M+H)⁺=349.1, HPLC Conditions: C. ¹H NMR (400 MHz, D₂O) δ 4.15-4.20 (m, 2H), 3.40-3.46 (m, 4H), 2.38-2.42 (m, 2H), 2.34-2.36 (m, 1H), 2.01-2.04 (m, 1H), 1.45 (s, 9H).

### Exemplary Embodiment 1aa40.6

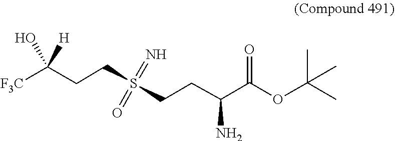 [SMILES: CC(C)(C)OC(=O)[C@@H](N)CC[S@@](=N)(=O)CC[C@@H](O)C(F)(F)F]

### tert-butyl (S)-2-amino-4-((R,3R)-4,4,4-trifluoro-3-hydroxybutylsulfonimidoyl)butanoate

To a mixture of (S)-2-amino-4-((R,3R)-4,4,4-trifluoro-3-hydroxybutylsulfonimidoyl)butanoic acid (Compound 328 (see also Exemplary Embodiment 1aa18.3), 350 mg, 1.20 mmol) in tert-butyl acetate (10 mL) was added HClO₄ (687 mg, 4.79 mmol, 414 μL, 70% purity) in one portion at 0° C. The mixture was stirred at 25° C. for 12 h. The mixture was cooled to 0° C. and adjusted to pH=8 with 2M aqueous NaOH solution and concentrated under vacuum. The residue was purified by prep-HPLC (column: Phenomenex C18 (75×30 mm, 3 μm); mobile phase: [water (FA)-MeCN]; gradient: 10-40% B, 10 min) to afford tert-butyl (S)-2-amino-4-((R,3R)-4,4,4-trifluoro-3-hydroxybutylsulfonimidoyl)butanoate (108 mg, 310 μmol, 25.9% yield)) as white solid. LCMS: Rt=1.598 min, (ES⁺) m/z (M+H)⁺=349.1, HPLC Conditions: C. ¹H NMR (400 MHz, D₂O) δ 4.13-4.20 (m, 2H), 3.36-3.48 (m, 4H), 2.38-2.41 (m, 2H), 2.35-2.37 (m, 1H), 2.04-2.06 (m, 1H), 1.45 (s, 9H).

The compounds described in Table 20 were prepared using the general methods outlined for the synthesis of Compounds 67-73 and the methods outlined above.

TABLE 20
Characterization of Compounds 482-492

| Compound | | MS | HPLC | HPLC |
|---|---|---|---|---|
| # | Structure | (M + H)+ | rt | cond. |
| 482 | 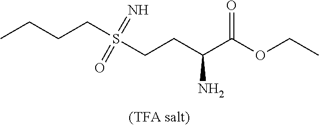 [SMILES: CCCCS(=N)(=O)CC[C@H](N)C(=O)OCC] | 251.0 | 1.302 | N |
| | ¹H NMR (400 MHz, D₂O) δ 4.32 (q, 3H), 3.81- | | | |
| | 3.57 (m, 2H), 3.55-3.45 (m, 2H), 2.59-2.38 (m, | | | |
| | 2H), 1.81 (quin, 2H), 1.47 (sxt, 2H), 1.29 (t, 3H), | | | |
| | 0.92 (t, 3H) | | | |
| 483 | 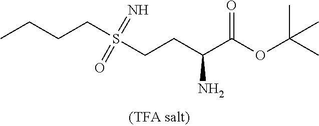 [SMILES: CCCCS(=N)(=O)CC[C@H](N)C(=O)OC(C)(C)C] | 279.1 | 2.572 | T |
| | ¹H NMR (400 MHz, D₂O) δ 4.21 (br t, 1H), 3.77- | | | |
| | 3.44 (m, 4H), 2.55-2.33 (m, 2H), 1.96-1.75 (m, | | | |
| | 2H), 1.58-1.38 (m, 11H), 0.94-0.93 (m, 1H), | | | |
| | 0.93 (br t, 2H). | | | |
| 484 | 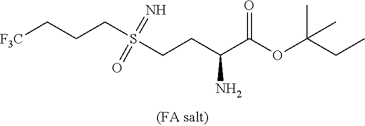 [SMILES: CCC(C)(C)OC(=O)[C@@H](N)CCS(=N)(=O)CCCC(F)(F)F] | 347.1 | 1.870 | C |
| | ¹H NMR (400 MHz, D₂O) δ 3.72-3.62 (m, 1H), | | | |
| | 3.39-3.24 (m, 4H), 2.46-2.30 (m, 2H), 2.26- | | | |
| | 2.01 (m, 4H), 1.79 (q, 2H), 1.43 (s, 6H), 0.86 (t, | | | |
| | 3H) | | | |
| 485 | 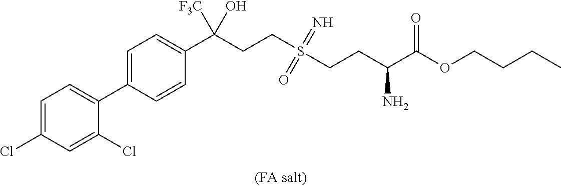 [SMILES: CCCCOC(=O)[C@@H](N)CCS(=N)(=O)CCC(O)(c1ccc(-c2ccc(Cl)cc2Cl)cc1)C(F)(F)F] | 569.1 | 2.502 | C |
| | ¹H NMR (400 MHz, MeOD-d₄) δ 8.36 (s, 1H), | | | |
| | 7.73 (d, 2H), 7.61 (d, 1H), 7.53 (d, 2H), 7.48- | | | |
| | 7.37 (m, 2H), 4.27-4.19 (m, 2H), 4.04-3.92 (m, | | | |
| | 1H), 3.39-3.34 (m, 1H), 3.31-3.18 (m, 2H), 2.91- | | | |
| | 2.71 (m, 2H), 2.70-2.55 (m, 1H), 2.42-2.27 (m, | | | |
| | 1H), 2.26-2.09 (m, 1H), 1.73-1.62 (m, 2H), 1.49- | | | |
| | 1.37 (m, 2H), 0.99-0.93 (m, 3H). | | | |
| 486 | 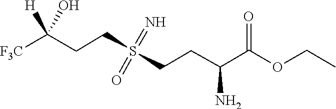 [SMILES: CCOC(=O)[C@@H](N)CC[S@@](=N)(=O)CC[C@@H](O)C(F)(F)F] | 321.0 | 2.040 | I |
| | ¹H NMR (400 MHz, D₂O) δ 4.27-4.16 (m, 3H), | | | |
| | 3.68 (br t, 1H), 3.44-3.28 (m, 4H), 2.31-2.17 (m, | | | |
| | 2H), 2.15-2.02 (m, 2H), 1.27-1.21 (m, 3H). | | | |
| 487 | 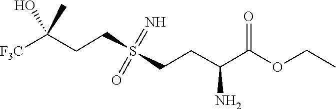 [SMILES: CCOC(=O)[C@@H](N)CC[S@@](=N)(=O)CC[C@@](C)(O)C(F)(F)F] | 335.1 | 1.647 | D |
| | ¹H NMR (400 MHz, D₂O) δ 4.20 (q, 2H), 3.72 (t, | | | |
| | 1H), 3.45-3.27 (m, 4H), 2.30-2.06 (m, 4H), 1.37 | | | |
| | (s, 3H), 1.24 (t, 3H) | | | |
| 488 | 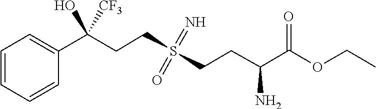 [SMILES: CCOC(=O)[C@@H](N)CC[S@@](=N)(=O)CC[C@](O)(c1ccccc1)C(F)(F)F] | 397.0 | 3.215 | I |
| | ¹H NMR (400 MHz, D₂O) δ 7.61 (br d, 2H), 7.54- | | | |
| | 7.46 (m, 3H), 4.21-4.13 (m, 2H), 3.64-3.57 (m, | | | |
| | 1H), 3.32-3.16 (m, 3H), 2.94-2.82 (m, 1H), 2.77- | | | |
| | 2.67 (m, 1H), 2.65-2.54 (m, 1H), 2.19-2.04 (m, | | | |
| | 1H), 2.00-1.87 (m, 1H), 1.22 (t, 3H). | | | |
| 489 | 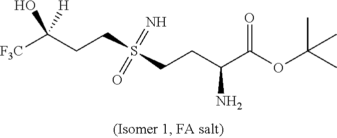 [SMILES: CC(C)(C)OC(=O)[C@@H](N)CC[S@@](=N)(=O)CC[C@H](O)C(F)(F)F] | 349.1 | 1.625 | C |
| | ¹H NMR (400 MHz, D₂O) δ 4.15-4.20 (m, 2H), | | | |
| | 3.40-3.46 (m, 4H), 2.38-2.42 (m, 2H), 2.34-2.36 | | | |
| | (m, 1H), 2.01-2.04 (m, 1H), 1.45 (s, 9H). | | | |
| 490 | 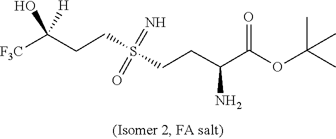 [SMILES: CC(C)(C)OC(=O)[C@@H](N)CC[S@](=N)(=O)CC[C@H](O)C(F)(F)F] | 349.1 | 1.603 | C |
| | ¹H NMR (400 MHz, D₂O) δ 4.14-4.21 (m, 2H), | | | |
| | 3.39-3.43 (m, 4H), 2.36-2.40 (m, 2H), 2.19-2.22 | | | |
| | (m, 1H), 2.04-2.06 (m, 1H), 1.45 (s, 9H). | | | |
| 491 | 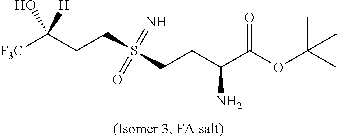 [SMILES: CC(C)(C)OC(=O)[C@@H](N)CC[S@@](=N)(=O)CC[C@@H](O)C(F)(F)F] | 349.1 | 1.598 | C |
| | ¹H NMR (400 MHz, D₂O) δ 4.13-4.20 (m, 2H), | | | |
| | 3.36-3.48 (m, 4H), 2.38-2.41 (m, 2H), 2.35-2.37 | | | |
| | (m, 1H), 2.04-2.06 (m, 1H), 1.45 (s, 9H) | | | |
| 492 | 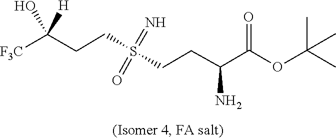 [SMILES: CC(C)(C)OC(=O)[C@@H](N)CC[S@](=N)(=O)CC[C@@H](O)C(F)(F)F] | 349.1 | 1.629 | C |
| | ¹H NMR (400 MHz, D₂O) δ 4.12-4.19 (m, 2H), | | | |
| | 3.37-3.45 (m, 4H), 2.33-2.38 (m, 2H), 2.19-2.22 | | | |
| | (m, 1H), 2.00-2.04 (m, 1H), 1.45 (s, 9H) | | | |

The compounds described in Table 21 were prepared using the general methods outlined for the synthesis of Compounds 100-105.

TABLE 21
Characterization of Compound 493

| Compound | | MS | HPLC | HPLC |
|---|---|---|---|---|
| # | Structure | (M + H)⁺ | Rt | cond. |
| 493 | 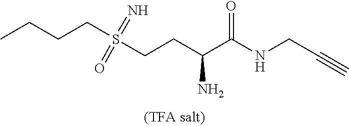 [SMILES: C#CCNC(=O)[C@@H](N)CCS(=N)(=O)CCCC] | 260.2 | 2.601 | G |
| | ¹H NMR (400 MHz, D₂O) δ 4.14 (br t, 1H), 4.07- | | | |
| | 3.90 (m, 2H), 3.53-3.32 (m, 4H), 2.60 (br s, 1H), | | | |
| | 2.39 (br d, 2H), 1.85-1.66 (m, 2H), 1.42 (br dd, | | | |
| | 2H), 0.88 (br t, 3H) | | | |

### Exemplary Embodiment 1aa41 (Isomer 1) and 1aa41 (Isomer 2)

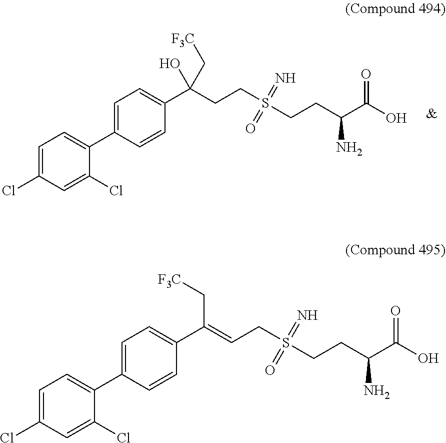 [SMILES: N=S(=O)(CC[C@H](N)C(=O)O)CCC(O)(CC(F)(F)F)c1ccc(-c2ccc(Cl)cc2Cl)cc1 ; N=S(=O)(C/C=C(\CC(F)(F)F)c1ccc(-c2ccc(Cl)cc2Cl)cc1)CC[C@H](N)C(=O)O]

### (2S)-2-amino-4-(3-(2′, 4′-dichloro-[1, 1′-biphenyl]-4-yl)-5, 5, 5-trifluoro-3-hydroxypentylsulfonimidoyl) butanoic acid & (2S)-2-amino-4-((E)-3-(2′, 4′-dichloro-[1, 1′-biphenyl]-4-yl)-5, 5, 5-trifluoropent-2-en-1-ylsulfonimidoyl) butanoic acid

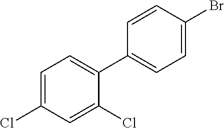 [SMILES: Clc1ccc(-c2ccc(Br)cc2)c(Cl)c1]

A mixture of 1-bromo-4-iodobenzene (2.5 g, 8.84 mmol, 1 eq), (2,4-dichlorophenyl)boronic acid (1.52 g, 7.95 mmol, 0.9 eq), Pd(dppf)Cl₂ (722 mg, 884 mol, 0.1 eq), and Na₂CO₃ (2.81 g, 26.5 mmol, 3 eq) in t-BuOH (18 mL) and H₂O (6 mL) was degassed and purged 3 times with N₂, and then the mixture was stirred at 90° C. for 2 hr under N₂ atmosphere. The mixture was poured into H₂O (40 mL) and extracted with EtOAc (40 mL×3). The combined organic layers were dried over Na₂SO₄, filtered, and concentrated under reduced pressure. The residue was purified by column chromatography (SiO₂, petroleum ether) to give 4′-bromo-2, 4-dichloro-1, 1′-biphenyl (2.56 g, 8.48 mmol, 95.9% yield) as a white solid. ¹H NMR (400 MHz, MeOD-d₄) δ 7.64-7.56 (m, 3H), 7.44-7.28 (m, 4H).

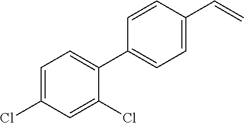 [SMILES: C=Cc1ccc(-c2ccc(Cl)cc2Cl)cc1]

To a solution of 4′-bromo-2, 4-dichloro-1, 1′-biphenyl (2.50 g, 8.28 mmol, 1 eq) and potassium vinyltrifluoroborate (5.54 g, 41.4 mmol, 5 eq) in t-BuOH (30 mL) and H₂O (10 mL) was added Pd(dppf)Cl₂ (676 mg, 828 μmol, 0.1 eq) and Na₂CO₃ (2.63 g, 24.8 mmol, 3 eq). The mixture was stirred at 90° C. for 16 hr. The mixture was poured into H₂O (40 mL) and extracted with EtOAc (40 mL×3). The combined organic layers were dried over Na₂SO₄, filtered, and concentrated under reduced pressure. The residue was purified by column chromatography (SiO₂, petroleum ether) to obtain 2,4-dichloro-4′-vinyl-1, 1′-biphenyl (1.9 g, 7.63 mmol, 92.1% yield) as a white solid. ¹H NMR (400 MHz, CDCl₃-d) δ 7.52-7.46 (m, 3H), 7.42-7.37 (m, 2H), 7.36-7.29 (m, 2H), 6.82-6.73 (m, 1H), 5.82 (dd, 1H), 5.35-5.29 (m, 1H).

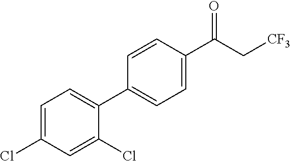 [SMILES: O=C(CC(F)(F)F)c1ccc(-c2ccc(Cl)cc2Cl)cc1]

A mixture of 2,4-dichloro-4′-vinyl-1, 1′-biphenyl (1.5 g, 6.02 mmol, 1 eq), sodium trifluoromethanesulfinate (1.88 g, 12.0 mmol, 1.88 mL, 2 eq), potassium persulfate (326 mg, 1.20 mmol, 241 μL, 0.2 eq), and silver nitrate (205 mg, 1.20 mmol, 0.2 eq) in DMF (20 mL) was stirred at 20° C. for 24 hr. The reaction mixture was diluted with MTBE and filtered through a celite pad. MTBE (50 mL) and water (50 mL) were added open to air, the organic layer was separated, and the aqueous layer was washed with MTBE (8×10 mL). The combined organic layers were dried over Na₂SO₄, filtered, and concentrated. The residue was purified by column chromatography (SiO₂, petroleum ether/Ethyl acetate=99:1) to obtain 1-(2′,4′-dichloro-[1,1′-biphenyl]-4-yl)-3,3,3-trifluoropropan-1-one (450 mg, 1.35 mmol, 22.4% yield) as a white solid. ¹H NMR (400 MHz, CDCl₃-d) δ 8.05-7.95 (m, 2H), 7.62-7.52 (m, 3H), 7.38-7.34 (m, 1H), 7.31-7.27 (m, 1H), 3.84 (q, 2H).

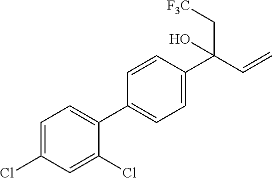 [SMILES: C=CC(O)(CC(F)(F)F)c1ccc(-c2ccc(Cl)cc2Cl)cc1]

To a solution of 1-(2′,4′-dichloro-[1,1′-biphenyl]-4-yl)-3,3,3-trifluoropropan-1-one (200 mg, 600 μmol, 1 eq) in THE (2 mL) was added bromo(vinyl)magnesium (1 M, 901 μL, 1.5 eq) at 0° C. The mixture was stirred at 0° C. for 1 h under N₂, and then the mixture was poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM/MeOH=10:1) to obtain 3-(2′, 4′-dichloro-[1, 1′-biphenyl]-4-yl)-5, 5, 5-trifluoropent-1-en-3-ol (130 mg, 360 μmol, 60.0% yield) as a colorless oil. ¹H NMR (400 MHz, CDCl₃-d) δ 7.56-7.49 (m, 3H), 7.45-7.41 (m, 2H), 7.33-7.28 (m, 2H), 6.36 (dd, 1H), 5.45 (d, 1H), 5.32 (d, 1H), 2.84 (q, 2H), 2.40 (d, 1H).

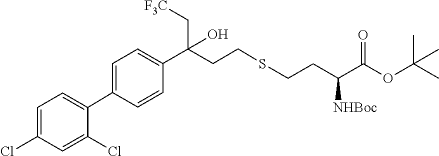 [SMILES: CC(C)(C)OC(=O)N[C@@H](CCSCCC(O)(CC(F)(F)F)c1ccc(-c2ccc(Cl)cc2Cl)cc1)C(=O)OC(C)(C)C]

A mixture of 3-(2′, 4′-dichloro-[1, 1′-biphenyl]-4-yl)-5, 5, 5-trifluoropent-1-en-3-ol (100 mg, 277 mol, 1 eq), tert-butyl (tert-butoxycarbonyl)-L-homocysteinate (121 mg, 415 μmol, 1.5 eq), and AIBN (6.82 mg, 41.5 μmol, 0.15 eq) in MeOH (0.3 mL)/H₂O (0.1 mL) was degassed and purged 3 times with Ar, and then the mixture was stirred at 60° C. for 16 h under N₂ atmosphere. The mixture was poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM/MeOH=10:1) to obtain tert-butyl N-(tert-butoxycarbonyl)-S-(3-(2′, 4′-dichloro-[1, 1′-biphenyl]-4-yl)-5, 5, 5-trifluoro-3-hydroxypentyl)-L-homocysteinate (138 mg, 211 μmol, 76.4% yield) as a colorless oil. ¹H NMR (400 MHz, CDCl₃-d) δ 7.53-7.40 (m, 5H), 7.33-7.29 (m, 2H), 5.20-5.03 (m, 1H), 4.35-4.22 (m, 1H), 2.75-2.66 (m, 2H), 2.58-2.45 (m, 3H), 2.39-2.24 (m, 3H), 2.04-1.96 (m, 1H), 1.91-1.81 (m, 1H), 1.47-1.44 (m, 18H).

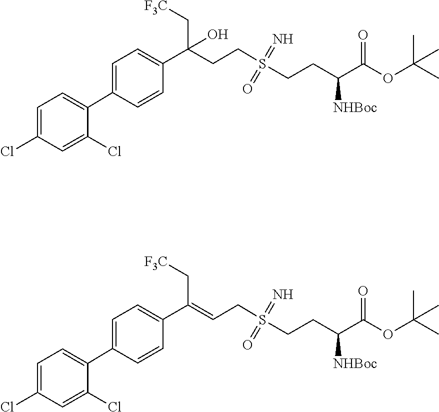 [SMILES: CC(C)(C)OC(=O)N[C@@H](CCS(=N)(=O)CCC(O)(CC(F)(F)F)c1ccc(-c2ccc(Cl)cc2Cl)cc1)C(=O)OC(C)(C)C ; CC(C)(C)OC(=O)N[C@@H](CCS(=N)(=O)C/C=C(\CC(F)(F)F)c1ccc(-c2ccc(Cl)cc2Cl)cc1)C(=O)OC(C)(C)C]

To a solution of tert-butyl N-(tert-butoxycarbonyl)-S-(3-(2′, 4′-dichloro-[1, 1′-biphenyl]-4-yl)-5, 5, 5-trifluoro-3-hydroxypentyl)-L-homocysteinate (138 mg, 211 μmol, 1 eq) in i-PrOH (2 mL) was added ammonium carbamate (132 mg, 1.69 mmol, 8 eq) and PhI(OAc)₂ (272 mg, 846 μmol, 4 eq). The mixture was stirred at 20° C. for 16 hr and then poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM/MeOH=10:1) to give tert-butyl (2S)-2-((tert-butoxycarbonyl) amino)-4-(3-(2′,4′-dichloro-[1,1′-biphenyl]-4-yl)-5,5,5-trifluoro-3-hydroxypentylsulfonimidoyl) butanoate (51 mg, 74.6 μmol, 35.3% yield) as a colorless oil and tert-butyl (2S)-2-((tert-butoxycarbonyl)amino)-4-((E)-3-(2′,4′-dichloro-[1,1′-biphenyl]-4-yl)-5,5,5-trifluoropent-2-en-1-ylsulfonimidoyl)butanoate (38 mg) as a colorless oil.

A solution of tert-butyl (2S)-2-((tert-butoxycarbonyl) amino)-4-(3-(2′,4′-dichloro-[1,1′-biphenyl]-4-yl)-5,5,5-trifluoro-3-hydroxypentylsulfonimidoyl) butanoate (51 mg, 74.6 μmol, 1 eq) in HCl/dioxane (5 M, 10 mL) was stirred at 30° C. for 4 h. The mixture was concentrated and the residue was purified by prep-HPLC (column: Phenomenex Luna C18 (100×30 mm, 3 μm); mobile phase: [H₂O (0.2% FA)-MeCN]; gradient: 20-60% B over 8.0 min) to give (2S)-2-amino-4-(3-(2′, 4′-dichloro-[1, 1′-biphenyl]-4-yl)-5, 5, 5-trifluoro-3-hydroxypentylsulfonimidoyl) butanoic acid (Isomer 1, 51 mg, 74.6 μmol, 35.3% yield) as a white solid. LCMS: Rt=2.230 min, (ES⁺) m/z (M+H)⁺=527.1, HPLC Conditions: C. ¹H NMR (400 MHz, MeOD-d₄) δ 7.63-7.56 (m, 3H), 7.48-7.36 (m, 4H), 3.74-3.64 (m, 1H), 3.38-3.32 (m, 1H), 3.28-3.12 (m, 2H), 2.97-2.79 (m, 2H), 2.75-2.62 (m, 1H), 2.57-2.37 (m, 2H), 2.34-2.19 (m, 2H).

A solution of tert-butyl (2S)-2-((tert-butoxycarbonyl)amino)-4-((E)-3-(2′,4′-dichloro-[1,1′-biphenyl]-4-yl)-5,5,5-trifluoropent-2-en-1-ylsulfonimidoyl)butanoate (38 mg, 57.1 μmol, 1 eq) in HCl/dioxane (5 M, 10 mL) was stirred at 20° C. for 4 h. The mixture was concentrated and the residue was purified by prep-HPLC: (column: Phenomenex Luna C18 (100*×30 mm, 3 μm); mobile phase: [H₂O (0.2% FA)-MeCN]; gradient: 20-60% B over 8.0 min) to give (2S)-2-amino-4-((E)-3-(2′, 4′-dichloro-[1, 1′-biphenyl]-4-yl)-5, 5, 5-trifluoropent-2-en-1-ylsulfonimidoyl) butanoic acid (Isomer 2, 3.18 mg, 6.24 μmol, 10.9% yield) as a white solid. LCMS: Rt=0.460 min, (ES⁺) m/z (M+H)⁺=508.9, HPLC Conditions: U. ¹H NMR (400 MHz, MeOD-d₄) δ 7.58 (s, 1H), 7.51 (d, 2H), 7.44-7.40 (m, 4H), 6.12-6.02 (m, 1H), 3.94 (br d, 1H), 3.71-3.62 (m, 1H), 3.52-3.42 (m, 2H), 3.35 (br s, 1H), 3.30-3.11 (m, 2H), 2.30-2.16 (m, 2H).

### Exemplary Embodiment 1aa42

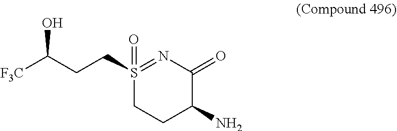 [SMILES: N[C@H]1CC[S@@](=O)(CC[C@H](O)C(F)(F)F)=NC1=O]

### (1R,4S)-4-amino-1-((S)-4,4,4-trifluoro-3-hydroxybutyl)-5,6-dihydro-116,2-thiazin-3 (4H)-one 1-oxide

To a solution of benzyl (S)-2-(((benzyloxy)carbonyl)amino)-4-((R,3R)-4,4,4-trifluoro-3-hydroxybutylsulfonimidoyl)butanoate (4.66 g, 9.02 mmol) in MeTHF (70 mL) and HCl (0.5 M, 15.34 mL) was added 10% Pd/C (4.66 g) under N₂. The suspension was degassed under vacuum and purged with H₂ several times. The mixture was stirred under H2 (50 psi) at 30° C. for 2 h. The reaction mixture was filtered, and the filtrate was concentrated. The mixture was purified by prep-HPLC (column: Phenomenex C18 (75×30 mm, 3 μm); mobile phase: [water (NH₄HCO₃)-MeCN]; gradient: 1-20% B, 10 min) to give (1R,4S)-4-amino-1-((S)-4,4,4-trifluoro-3-hydroxybutyl)-5,6-dihydro-116,2-thiazin-3 (4H)-one 1-oxide ((1.24 mg, 4.52 μmol) as white solid. LCMS: Rt=0.516 min., (ES⁺) m/z (M+H)⁺=275.0, HPLC Conditions: A. ¹H NMR (400 MHz, D₂O) δ 4.12-4.31 (m, 2H), 3.48-3.81 (m, 4H), 2.44-2.50 (m, 4H).

### Exemplary Embodiment 1aa43

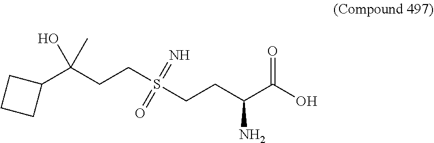 [SMILES: CC(O)(CCS(=N)(=O)CC[C@H](N)C(=O)O)C1CCC1]

### (2S)-2-amino-4-(3-cyclobutyl-3-hydroxybutylsulfonimidoyl)butanoic acid

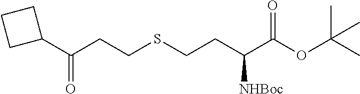 [SMILES: CC(C)(C)OC(=O)N[C@@H](CCSCCC(=O)C1CCC1)C(=O)OC(C)(C)C]

To a solution of (S)-tert-butyl 2-((tert-butoxycarbonyl)amino)-4-mercaptobutanoate (1 g, 3.43 mmol, 1 eq) and TEA (1.04 g, 10.3 mmol, 1.43 mL, 3 eq) in THE (10 mL) was added 1-cyclobutylprop-2-en-1-one (1.8 g, 16.3 mmol) under N₂ atmosphere. The mixture was stirred at 25° C. for 16 h. The reaction mixture was concentrated under reduced pressure to give a residue. The residue was purified by column chromatography (SiO₂, petroleum ether/EtOAc=1:0 to 1:1) to give (S)-tert-butyl 2-((tert-butoxycarbonyl)amino)-4-((3-cyclobutyl-3-oxopropyl) thio)butanoate (1.5 g, crude) as a yellow oil. ¹H NMR (400 MHz, CDCl₃-d) δ 5.10 (br d, 1H), 4.33-4.12 (m, 1H), 2.90 (s, 1H), 2.77-2.72 (m, 2H), 2.64 (s, 2H), 2.56-2.52 (m, 2H), 2.15 (td, 6H), 1.85-1.81 (m, 2H), 1.47 (s, 9H), 1.45 (s, 9H).

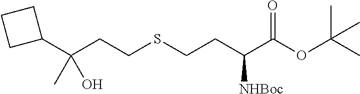 [SMILES: CC(C)(C)OC(=O)N[C@@H](CCSCCC(C)(O)C1CCC1)C(=O)OC(C)(C)C]

To a solution of (S)-tert-butyl 2-((tert-butoxycarbonyl)amino)-4-((3-cyclobutyl-3-oxopropyl)thio)butanoate (500 mg, 1.25 mmol, 1 eq) in THE (5 mL) was added MeMgBr (3 M, 207 uL, 0.5 eq) at 0° C. The mixture was stirred at 0° C. for 1 h. The reaction mixture was poured into saturated aqueous NH₄Cl (10 mL) and extracted with EtOAc (8 mL×2). The combined organic layers were washed with brine (10 mL), dried over Na₂SO₄, filtered, and concentrated under reduced pressure to give a residue. The residue was purified by column chromatography (SiO₂, petroleum ether/EtOAc=1:0 to 3:1) to give (2S)-tert-butyl 2-((tert-butoxycarbonyl)amino)-4-((3-cyclobutyl-3-hydroxybutyl)thio)butanoate (320 mg, 766 μmol, 61.5% yield) as a yellow oil. ¹H NMR (400 MHz, CDCl₃-d) δ 5.19-5.07 (m, 1H), 4.29-4.22 (m, 1H), 2.64-2.53 (m, 4H), 2.43-2.32 (m, 1H), 2.00-1.78 (m, 6H), 1.75-1.68 (m, 4H), 1.49-1.42 (m, 18H), 1.11-1.06 (m, 3H).

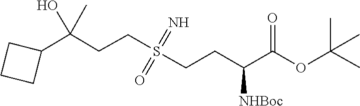 [SMILES: CC(C)(C)OC(=O)N[C@@H](CCS(=N)(=O)CCC(C)(O)C1CCC1)C(=O)OC(C)(C)C]

A mixture of (2S)-tert-butyl 2-((tert-butoxycarbonyl)amino)-4-((3-cyclobutyl-3-hydroxybutyl)thio)butanoate (170 mg, 407 μmol, 1 eq), ammonium carbamate (254 mg, 3.26 mmol, 8 eq) and PhI(OAc)₂ (524 mg, 1.63 mmol, 4 eq) in i-PrOH (2 mL) was stirred at 25° C. for 12 h. The reaction mixture was concentrated under reduced pressure and the residue was diluted with water (10 mL) and extracted with EtOAc (5 mL×2). The combined organic layers were washed with brine (10 mL), dried over Na₂SO₄, filtered, and concentrated under reduced pressure to give a residue. The residue was purified by prep-TLC (SiO₂, petroleum ether/EtOAc=1:5) to give (2S)-tert-butyl 2-((tert-butoxycarbonyl)amino)-4-(3-cyclobutyl-3-hydroxybutylsulfonimidoyl)butanoate (50 mg, 111 μmol, 27.3% yield) as a yellow oil. ¹H NMR (400 MHz, CDCl₃-d) δ 5.29-5.21 (m, 1H), 4.35-4.22 (m, 1H), 3.32-3.00 (m, 4H), 2.15-2.09 (m, 1H), 2.05-1.68 (m, 10H), 1.49 (s, 9H), 1.46 (s, 9H), 1.09 (s, 3H).

A mixture of (2S)-tert-butyl 2-((tert-butoxycarbonyl)amino)-4-(3-cyclobutyl-3-hydroxybutylsulfonimidoyl)butanoate (42 mg, 93.6 μmol, 1 eq) in HCl/dioxane (20 mL, 4 M) was stirred at 20° C. for 16 h. The reaction mixture was concentrated under reduced pressure to give and the residue was purified by prep-HPLC (column: C18-1 (150×30 mm, 5 μm); mobile phase: [water(FA)-ACN]; gradient: 1-30% B, 10 min) to give (2S)-2-amino-4-(3-cyclobutyl-3-hydroxybutylsulfonimidoyl)butanoic acid (9.48 mg, 28.0 μmol, 29.9% yield, 100% purity, FA), as white solid. LCMS: Rt=1.560 min, (ES⁺) m/z (M+H)⁺=293.2, HPLC Conditions: I. ¹H NMR (400 MHz, D₂O) δ 3.93 (t, 1H), 3.74-3.39 (m, 4H), 2.52-2.33 (m, 3H), 1.96-1.74 (m, 7H), 1.69-1.58 (m, 1H), 1.10 (s, 3H).

### Exemplary Embodiment 1aa44

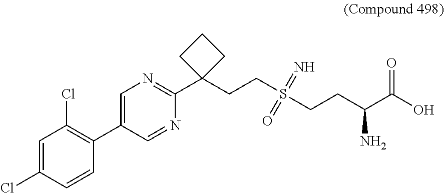 [SMILES: N=S(=O)(CC[C@H](N)C(=O)O)CCC1(c2ncc(-c3ccc(Cl)cc3Cl)cn2)CCC1]

### (2S)-2-amino-4-(2-(1-(5-(2, 4-dichlorophenyl) pyrimidin-2-yl) cyclobutyl) ethylsulfonimidoyl) butanoic acid

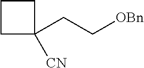 [SMILES: N#CC1(CCOCc2ccccc2)CCC1]

To a solution of LDA (2 M, 18.5 mL, 2 eq) in THE (15 mL) was added cyclobutanecarbonitrile (1.5 g, 18.5 mmol, 1 eq) at −65° C. and the mixture was stirred for 0.5 h, then 2-bromoethoxymethylbenzene (4.02 g, 18.7 mmol, 2.95 mL, 1.01 eq) in THE (20 mL) was added and the mixture was stirred at −65° C. for 1h. The mixture was poured into H₂O (100 mL) and extracted with EtOAc (100 mL×2). The combined organic layers were dried over Na₂SO₄, filtered, and concentrated under reduced pressure. The residue was purified by column chromatography (SiO₂, petroleum ether/EtOAc=49:1 to 5:1) to give 1-(2-(benzyloxy) ethyl) cyclobutane-1-carbonitrile (3 g, 13.9 mmol, 75.4% yield) as a colorless oil. ¹H NMR (400 MHz, CDCl₃-d) δ 7.39-7.28 (m, 5H), 4.53 (s, 2H), 3.66 (t, 2H), 2.61-2.46 (m, 2H), 2.27-2.13 (m, 3H), 2.10-2.04 (m, 3H).

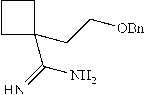 [SMILES: N=C(N)C1(CCOCc2ccccc2)CCC1]

A solution of trimethylaluminum (2 M, 15.0 mL, 2.15 eq) was added to a stirred suspension of NH₄Cl (1.49 g, 27.9 mmol, 2 eq) in toluene (20 mL) at 0° C. under N₂. After the addition, the ice water bath was removed, and the mixture was stirred at 25° C. for 2 h. 1-(2-(benzyloxy) ethyl) cyclobutane-1-carbonitrile (3 g, 13.9 mmol, 1 eq) was added as a solution in toluene (30 mL) and the mixture was stirred at 80° C. under N₂ for 12 h. The mixture was cooled with an ice water bath at 0° C. and quenched with 100 mL of methanol and stirred at 25° C. for 2 h. The mixture was filtered and washed with methanol. The combined filtrate was concentrated to afford crude 1-(2-(benzyloxy) ethyl) cyclobutane-1-carboximidamide (4.5 g, crude), as a white solid. ¹H NMR (400 MHz, MeOD-d₄) δ 7.46-7.18 (m, 5H), 4.57-4.42 (m, 2H), 3.67-3.45 (m, 2H), 2.52-2.01 (m, 6H).

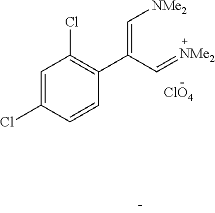

POCl₃ (6.73 g, 43.9 mmol, 4.09 mL, 0.6 eq) was added over 1 h to DMF (30 mL) while keeping the temperature at 20° C. After the solution has stirred at 20° C. for 1 h, 2-(2, 4-dichlorophenyl) acetic acid (3 g, 14.6 mmol, 0.2 eq) was added. The solution was warmed to 85° C. and stirred for 18 h. After the addition, the solution was cooled to 20° C. and poured onto 36 g of ice with vigorous stirring. A solution of sodium perchlorate (3.75 M, 5.85 mL, 0.3 eq) in H₂O (6 mL) was added, and a crystalline precipitate formed over 10 min. The precipitate was filtered, washed with water, and the reaction mixture was concentrated under reduced pressure to give (Z)—N-(2-(2, 4-dichlorophenyl)-3-(dimethylamino) allylidene)-N-methylmethanaminium (2.8 g, 10.3 mmol, 14.1% yield) as a brown solid. ¹H NMR (400 MHz, CDCl₃-d) δ 8.02 (s, 2H), 7.52 (d, 1H), 7.40-7.29 (m, 2H), 3.39 (s, 6H), 2.54 (s, 6H).

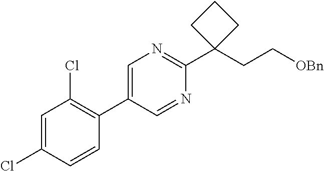 [SMILES: Clc1ccc(-c2cnc(C3(CCOCc4ccccc4)CCC3)nc2)c(Cl)c1]

NaOMe (5 M, 1.16 mL, 2 eq) was added dropwise to a stirred suspension of (Z)—N-(2-(2, 4-dichlorophenyl)-3-(dimethylamino) allylidene)-N-methylmethanaminium (0.79 g, 2.90 mmol, 1 eq) in MeOH (10 mL) at 20° C. and the mixture was stirred at 20° C. for 5 h. The mixture was poured into H₂O (40 mL) and extracted with EtOAc (40 mL×3). The combined organic layers were dried over Na₂SO₄, filtered, and concentrated under reduced pressure. The residue was purified by column chromatography (SiO₂, petroleum ether/EtOAc=49:1 to 5:1) to give 2-(1-(2-(benzyloxy) ethyl) cyclobutyl)-5-(2, 4-dichlorophenyl) pyrimidine (293 mg, crude) as a colorless oil. ¹H NMR (400 MHz, MeOD-d₄) δ 8.74 (s, 2H), 7.65 (d, 1H), 7.45 (dd, 1H), 7.26-7.17 (m, 4H), 7.15-7.10 (m, 2H), 4.27 (s, 2H), 3.43 (t, 2H), 2.72-2.63 (m, 2H), 2.40 (t, 2H), 2.29-2.19 (m, 2H), 2.15-2.06 (m, 1H), 1.94-1.84 (m, 1H).

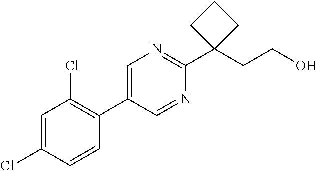 [SMILES: OCCC1(c2ncc(-c3ccc(Cl)cc3Cl)cn2)CCC1]

To a solution of 2-(1-(2-(benzyloxy) ethyl) cyclobutyl)-5-(2, 4-dichlorophenyl) pyrimidine (179 mg, 433 μmol, 1 eq) in DCM (3 mL) was added BCl₃ (507 mg, 4.33 mmol, 563 μL, 10 q). After the addition, the mixture was stirred at 0° C. for 0.25 h. The mixture was poured into H₂O (30 mL) and extracted with DCM (30 mL×3). The combined organic layers were dried over Na₂SO₄, filtered, and concentrated under reduced pressure. The residue was purified by column chromatography (SiO₂, petroleum ether/EtOAc=49:1 to 1:1). The residue was further purified by prep-TLC (DCM/MeOH=10:1) to give 2-(1-(5-(2, 4-dichlorophenyl) pyrimidin-2-yl) cyclobutyl) ethan-1-ol (259 mg, crude) as a pink solid. ¹H NMR (400 MHz, MeOD-d₄) δ 8.82 (s, 2H), 7.68 (d, 1H), 7.52-7.47 (m, 2H), 3.41 (t, 2H), 2.75-2.61 (m, 2H), 2.38-2.28 (m, 2H), 2.27-2.20 (m, 2H), 2.16-2.07 (m, 1H), 1.94-1.85 (m, 1H).

To a solution of 2-(1-(5-(2, 4-dichlorophenyl) pyrimidin-2-yl) cyclobutyl) ethan-1-ol (70 mg, 217 μmol, 1 eq) in DCM (2 mL) was added imidazole (31.0 mg, 455 μmol, 2.1 eq) and 1,1′-(azodicarbonyl)dipiperidine (115 mg, 455 μmol, 2.1 eq). Then trimethylphosphane (1 M, 433 μL, 2 eq) and tert-butyl (2S)-2-(tert-butoxycarbonylamino)-4-sulfanyl-butanoate (126 mg, 433 μmol, 2 eq) was added at 0° C. under Ar, the resulting mixture was stirred at 20° C. for 3 h under Ar. The mixture was poured into H₂O (30 mL) and extracted with DCM (30 mL×3). The combined organic layers were dried over Na₂SO₄, filtered, and concentrated under reduced pressure. The residue was purified by prep-TLC (DCM:MeOH=10:1) to obtain tert-butyl N-(tert-butoxycarbonyl)-S-(2-(1-(5-(2, 4-dichlorophenyl) pyrimidin-2-yl) cyclobutyl) ethyl)-L-homocysteinate (92 mg, 154 μmol, 71.2% yield) as a colorless oil.

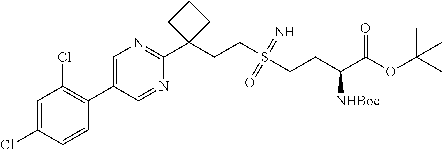 [SMILES: CC(C)(C)OC(=O)N[C@@H](CCS(=N)(=O)CCC1(c2ncc(-c3ccc(Cl)cc3Cl)cn2)CCC1)C(=O)OC(C)(C)C]

A mixture of tert-butyl N-(tert-butoxycarbonyl)-S-(2-(1-(5-(2, 4-dichlorophenyl) pyrimidin-2-yl) cyclobutyl) ethyl)-L-homocysteinate (92 mg, 154 μmol, 1 eq), PhI(OAc)₂ (199 mg, 617 μmol, 4 eq) and ammonium carbamate (96.3 mg, 1.23 mmol, 8 eq) in i-PrOH (2 mL) was stirred at 25° C. for 12 h. The mixture was poured into H₂O (10 mL) and extracted with EtOAc (10 mL×3). The combined organic layers were dried over Na₂SO₄, filtered, and concentrated under reduced pressure. The residue was purified by prep-TLC (DCM:MeOH=10:1) to give tert-butyl (2S)-2-((tert-butoxycarbonyl) amino)-4-(2-(1-(5-(2, 4-dichlorophenyl) pyrimidin-2-yl) cyclobutyl) ethylsulfonimidoyl) butanoate (13.8 mg, 22.0 μmol, 14.3% yield) as a colorless oil.

A mixture of give tert-butyl (2S)-2-((tert-butoxycarbonyl) amino)-4-(2-(1-(5-(2, 4-dichlorophenyl) pyrimidin-2-yl) cyclobutyl) ethylsulfonimidoyl) butanoate (13.8 mg, 22.0 μmol, 1 eq) in HCl/dioxane (8 mL) was stirred at 30° C. for 4 h under N₂ atmosphere. The reaction mixture was concentrated under reduced pressure. The residue was purified by prep-HPLC (column: Phenomenex Luna C18 (100×40 mm, 5 μm); mobile phase: [H₂O (0.2% FA)-MeCN]; gradient: 1%-40% B over 8.0 min) to give (2S)-2-amino-4-(2-(1-(5-(2, 4-dichlorophenyl) pyrimidin-2-yl) cyclobutyl) ethylsulfonimidoyl) butanoic acid (6.1 mg, 12.9 μmol, 58.9% yield) as a white solid.

LCMS: Rt=2.027 min, (ES⁺) m/z (M+H)⁺=471.1, HPLC Conditions: C. ¹H NMR (400 MHz, MeOD-d₄) δ 8.89-8.86 (m, 2H), 8.28 (s, 1H), 7.68 (s, 1H), 7.51 (s, 2H), 3.70 (td, 1H), 3.46-3.34 (m, 1H), 3.29-3.23 (m, 1H), 3.09-3.02 (m, 2H), 2.74-2.67 (m, 2H), 2.61-2.54 (m, 2H), 2.34 (ddd, 2H), 2.26-2.15 (m, 3H), 2.04-1.96 (m, 1H).

### Exemplary Embodiment 1aa45

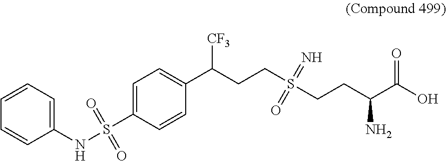 [SMILES: N=S(=O)(CCC(c1ccc(S(=O)(=O)Nc2ccccc2)cc1)C(F)(F)F)CC[C@H](N)C(=O)O]

### (2S)-2-amino-4-(4,4,4-trifluoro-3-(4-(N-phenylsulfamoyl)phenyl)butylsulfonimidoyl)butanoic acid

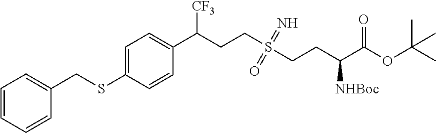 [SMILES: CC(C)(C)OC(=O)N[C@@H](CCS(=N)(=O)CCC(c1ccc(SCc2ccccc2)cc1)C(F)(F)F)C(=O)OC(C)(C)C]

To a solution of tert-butyl (2S)-4-(3-(4-bromophenyl)-4,4,4-trifluorobutylsulfonimidoyl)-2-((tert-butoxycarbonyl)amino)butanoate (400 mg, 681 μmol, 1 eq) in dioxane (4 mL) was added phenylmethanethiol (254 mg, 2.04 mmol, 240 μL, 3 eq), Pd₂(dba)₃ (93.5 mg, 102 μmol, 0.15 eq), DIEA (264 mg, 2.04 mmol, 356 μL, 3 eq) and Xantphos (177 mg, 306 μmol, 0.45 eq) at 25° C. The mixture was stirred at 110° C. for 8 h. The reaction mixture was poured into water (50 mL) and extracted with EtOAc (50 mL×2). The combined organic layers were washed with brine (50 mL), dried over Na₂SO₄, filtered, and concentrated under reduced pressure to give a residue. The residue was purified by column chromatography (SiO₂, petroleum ether/EtOAc=95:5 to 50:50) to give tert-butyl (2S)-4-(3-(4-(benzylthio)phenyl)-4,4,4-trifluorobutylsulfonimidoyl)-2-((tert-butoxycarbonyl)amino)butanoate (350 mg, 555 μmol, 81.5% yield) as a yellow oil. ¹H NMR (400 MHz, CDCl₃-d) δ 7.35-7.28 (m, 7H), 7.20 (d, 2H), 5.37-5.22 (m, 1H), 4.33-4.21 (m, 1H), 3.56-3.42 (m, 1H), 3.37-3.22 (m, 2H), 3.21-3.02 (m, 2H), 3.00-2.82 (m, 1H), 2.68-2.56 (m, 1H), 2.43-2.34 (m, 2H), 2.19-2.06 (m, 2H), 1.48 (s, 9H), 1.45 (s, 9H).

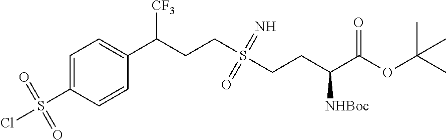 [SMILES: CC(C)(C)OC(=O)N[C@@H](CCS(=N)(=O)CCC(c1ccc(S(=O)(=O)Cl)cc1)C(F)(F)F)C(=O)OC(C)(C)C]

To a solution of tert-butyl (2S)-4-(3-(4-(benzylthio)phenyl)-4,4,4-trifluorobutylsulfonimidoyl)-2-((tert-butoxycarbonyl)amino)butanoate (30 mg, 47.6 μmol, 1 eq) in MeCN (0.5 mL) was added AcOH (8.57 mg, 143 μmol, 8.17 μL, 3 eq), H₂O (9.42 mg, 523 μmol, 9.42 μL, 11 eq) and sulfuryl chloride (19.3 mg, 143 μmol, 14.3 μL, 3 eq) at 0° C. The mixture was stirred at 17° C. for 1 h. The reaction mixture was concentrated under reduced pressure to give tert-butyl (2S)-2-((tert-butoxycarbonyl)amino)-4-(3-(4-(chlorosulfonyl)phenyl)-4,4,4-trifluorobutylsulfonimidoyl)butanoate (115 mg, 189 μmol, 99.6% yield), as a yellow oil and used directly.

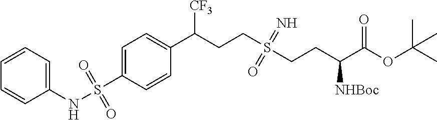 [SMILES: CC(C)(C)OC(=O)N[C@@H](CCS(=N)(=O)CCC(c1ccc(S(=O)(=O)Nc2ccccc2)cc1)C(F)(F)F)C(=O)OC(C)(C)C]

To a solution of aniline (3.44 mg, 36.9 μmol, 3.36 μL, 0.8 eq) and TEA (14.0 mg, 138 μmol, 19.3 μL, 3 eq) in DCM (2 mL) was added tert-butyl (2S)-2-((tert-butoxycarbonyl)amino)-4-(3-(4-(chlorosulfonyl)phenyl)-4,4,4-trifluorobutylsulfonimidoyl)butanoate (28 mg, 46.1 μmol, 1 eq) at 17° C. The mixture was stirred at 17° C. for 1 h. The reaction mixture was poured into water (30 mL) and extracted with DCM (30 mL×2). The combined organic layers were washed with brine (30 mL), dried over Na₂SO₄, filtered, and concentrated under reduced pressure to give a residue. The residue was purified by prep-TLC (SiO₂, petroleum ether:EtOAc=1:1) to give tert-butyl (2S)-2-((tert-butoxycarbonyl)amino)-4-(4,4,4-trifluoro-3-(4-(N-phenylsulfamoyl)phenyl)butylsulfonimidoyl)butanoate (10 mg, 15.1 μmol, 10.9% yield) as a yellow oil.

A mixture of tert-butyl (2S)-2-((tert-butoxycarbonyl)amino)-4-(4,4,4-trifluoro-3-(4-(N-phenylsulfamoyl)phenyl)butylsulfonimidoyl)butanoate (10 mg, 15.1 μmol, 1 eq) in HCl/dioxane (5 mL, 4M) was stirred at 30° C. for 8 h. The reaction mixture was concentrated under reduced pressure and the residue was purified by prep-HPLC (column: Waters Xbridge C₁₈ (150×50 mm, 10 μm); mobile phase: [H₂O (10 mM NH₄HCO₃)-MeCN]; gradient:10-40% B over 8.0 min) to give (2S)-2-amino-4-(4,4,4-trifluoro-3-(4-(pyridin-3-yl)phenyl)butylsulfonimidoyl) butanoic acid (6 mg, 11.1 μmol, 73.8% yield) as a yellow oil. LCMS: Rt=2.228 min, (ES⁺) m/z (M+H)⁺=508.1, HPLC Conditions: B. ¹H NMR (400 MHz, MeOD-d₄) δ 7.79 (d, 2H), 7.53 (d, 2H), 7.27-7.18 (m, 2H), 7.12-7.03 (m, 3H), 3.83 (ddd, 1H), 3.70-3.64 (m, 1H), 3.29-3.17 (m, 2H), 3.13-3.02 (m, 1H), 2.85-2.72 (m, 1H), 2.62-2.50 (m, 1H), 2.49-2.37 (m, 1H), 2.33-2.22 (m, 2H).

### Exemplary Embodiment 1aa46

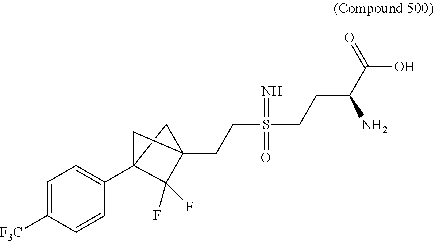 [SMILES: N=S(=O)(CC[C@H](N)C(=O)O)CCC12CC(c3ccc(C(F)(F)F)cc3)(C1)C2(F)F]

### (2S)-2-amino-4-(2-(2,2-difluoro-3-(4-(trifluoromethyl)phenyl)bicyclo[1.1.1]pentan-1-yl)ethylsulfonimidoyl)butanoic acid

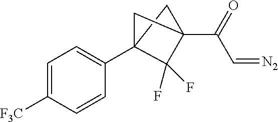

To a solution of 2,2-difluoro-3-(4-(trifluoromethyl)phenyl)bicyclo[1.1.1]pentane-1-carboxylic acid (750 mg, 2.57 mmol, 1 eq) in DCM (2 mL) was added oxalyl chloride (652 mg, 5.13 mmol, 449 μL, 2 eq) and DMF (9.38 mg, 128 μmol, 9.87 μL, 0.05 eq) at 0° C. The mixture was stirred at 0° C. for 2 h. The reaction mixture was concentrated under reduced pressure to give 2,2-difluoro-3-(4-(trifluoromethyl)phenyl)bicyclo[1.1.1]pentane-1-carbonyl chloride (790 mg, 2.54 mmol, 99.1% yield) as a yellow oil.

To a solution of 2,2-difluoro-3-(4-(trifluoromethyl)phenyl)bicyclo[1.1.1]pentane-1-carbonyl chloride (790 mg, 2.54 mmol, 1 eq) and TEA (643 mg, 6.36 mmol, 884 μL, 2.5 eq) in MeCN (5 mL) and THF (5 mL) was added diazomethyl(trimethyl)silane (2 M, 2.54 mL, 2 eq). The mixture was stirred at 20° C. for 30 min. After evaporation, EtOAc (15 mL) was added and the mixture was washed with 0.5 M aqueous citric acid (15 mL), 5% aqueous NaHCO₃ solution (15 mL) and brine (5 mL). The organic layer was dried over anhydrous Na₂SO₄, filtered, and concentrated under reduced pressure to give a residue. The residue was purified by prep-TLC (SiO₂, petroleum ether/EtOAc=3:1) to give 2-diazo-1-(2,2-difluoro-3-(4-(trifluoromethyl)phenyl)bicyclo[1.1.1]pentan-1-yl)ethan-1-one (260 mg, 822 μmol, 32.3% yield) as a yellow oil. ¹H NMR (400 MHz, CDCl₃-d) δ 7.64 (d, 2H), 7.40 (d, 2H), 5.51 (s, 1H), 2.61 (s, 2H), 2.16 (t, 2H).

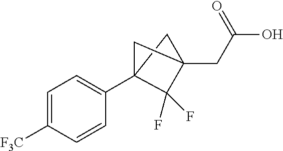 [SMILES: O=C(O)CC12CC(c3ccc(C(F)(F)F)cc3)(C1)C2(F)F]

To a solution of silver benzoate (37.7 mg, 164 μmol, 0.2 eq) in TEA (333 mg, 3.29 mmol, 458 μL, 4 eq) was added 2-diazo-1-(2,2-difluoro-3-(4-(trifluoromethyl)phenyl)bicyclo[1.1.1]pentan-1-yl)ethan-1-one (260 mg, 822 μmol, 1 eq) in THE (4 mL) and H₂O (1 mL). The resulting mixture was stirred at 20° C. for 30 min under irradiation with ultrasounds. The reaction mixture was filtered and concentrated under reduced pressure. The residue was purified by prep-TLC (SiO₂, petroleum ether/EtOAc=3:1) to give 2-(2,2-difluoro-3-(4-(trifluoromethyl)phenyl)bicyclo[1.1.1]pentan-1-yl)acetic acid (137 mg, 447 μmol, 54.4% yield) was a yellow oil. ¹H NMR (400 MHz, CDCl₃-d) δ 7.54 (d, 2H), 7.31 (d, 2H), 2.75 (s, 2H), 2.34 (s, 2H), 1.92 (t, 2H).

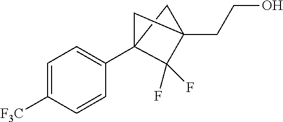 [SMILES: OCCC12CC(c3ccc(C(F)(F)F)cc3)(C1)C2(F)F]

To a solution of 2-(2,2-difluoro-3-(4-(trifluoromethyl)phenyl)bicyclo[1.1.1]pentan-1-yl)acetic acid (167 mg, 545 μmol, 1 eq) in THE (3 mL) was added BH₃·THF (1 M, 1.09 mL, 2 eq) at −15° C. The mixture was stirred at −15° C. for 2 h. The reaction mixture was then neutralized with diluted acetic acid (1 mL) and the organic solvent partially removed in vacuo. The residue was diluted with saturated aqueous NaHCO₃ solution (10 mL×2) and the solution was extracted with EtOAc (10×2 mL). The combined organic phases were dried over anhydrous Na₂SO₄, filtered and the solvent evaporated to give 2-(2,2-difluoro-3-(4-(trifluoromethyl)phenyl)bicyclo[1.1.1]pentan-1-yl)ethan-1-ol (157 mg, 537 μmol, 98.5% yield) as a yellow oil. ¹H NMR (400 MHz, CDCl₃-d) δ 7.61 (d, 2H), 7.44-7.34 (m, 2H), 3.82 (t, 2H), 2.34 (s, 2H), 2.00 (t, 2H), 1.80 (t, 2H).

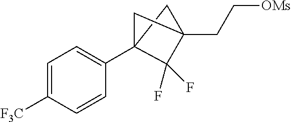 [SMILES: CS(=O)(=O)OCCC12CC(c3ccc(C(F)(F)F)cc3)(C1)C2(F)F]

To a solution of 2-(2,2-difluoro-3-(4-(trifluoromethyl)phenyl)bicyclo[1.1.1]pentan-1-yl)ethan-1-ol (157 mg, 537 μmol, 1 eq) in DCM (2 mL) was added methylsulfonyl methanesulfonate (187 mg, 1.07 mmol, 2 eq) and TEA (163 mg, 1.61 mmol, 224 μL, 3 eq) at 0° C. The mixture was stirred at 20° C. for 2 h. The reaction mixture was concentrated under reduced pressure, diluted with H₂O (3 mL), and extracted with DCM (3 mL×3). The combined organic phase was washed with brine (1 mL), dried with anhydrous Na₂SO₄, filtered, and concentrated under reduced pressure to give a residue to give 2-(2,2-difluoro-3-(4-(trifluoromethyl)phenyl)bicyclo[1.1.1]pentan-1-yl)ethyl methanesulfonate (190 mg, 513 μmol, 95.5% yield) as a yellow oil. ¹H NMR (400 MHz, CDCl₃-d) δ 7.61 (d, 2H), 7.39 (d, 2H), 4.36 (t, 2H), 3.06 (s, 3H), 2.37 (s, 2H), 2.21 (t, 2H), 1.86 (t, 2H), 1.41 (t, 3H).

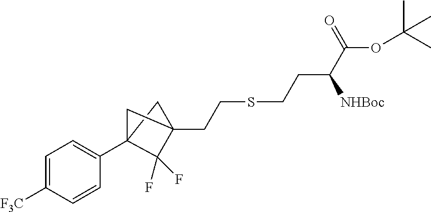 [SMILES: CC(C)(C)OC(=O)N[C@@H](CCSCCC12CC(c3ccc(C(F)(F)F)cc3)(C1)C2(F)F)C(=O)OC(C)(C)C]

A mixture of 2-(2,2-difluoro-3-(4-(trifluoromethyl)phenyl)bicyclo[1.1.1]pentan-1-yl)ethyl methanesulfonate (190 mg, 513 μmol, 1 eq), tert-butyl (2S)-2-(tert-butoxycarbonylamino)-4-sulfanyl-butanoate (194 mg, 667 μmol, 1.3 eq), KI (256 mg, 1.54 mmol, 3 eq), and K₂CO₃ (319 mg, 2.31 mmol, 4.5 eq) in DMF (2 mL) was degassed and purged with Ar 3 times, and then the mixture was stirred at 60° C. for 12 h under N₂ atmosphere. The residue was diluted with H₂O (10 mL), extracted with EtOAc (10 mL×3). The combined organic phase was washed with brine (10 mL), dried with anhydrous Na₂SO₄, filtered, and concentrated under reduced pressure to give a residue. The residue was purified by prep-TLC (SiO₂, petroleum ether/EtOAc=3:1) to give tert-butyl N-(tert-butoxycarbonyl)-S-(2-(2,2-difluoro-3-(4-(trifluoromethyl)phenyl)bicyclo[1.1.1]pentan-1-yl)ethyl)-L-homocysteinate (178 mg, 315 μmol, 61.3% yield) as a yellow oil. ¹H NMR (400 MHz, CDCl₃-d) δ 7.53 (d, 2H), 7.30 (d, 2H), 5.04 (br d, 1H), 4.22 (br d, 1H), 2.63-2.44 (m, 4H), 2.23 (s, 2H), 2.07-1.97 (m, 1H), 1.97-1.91 (m, 2H), 1.86-1.68 (m, 3H), 1.42-1.37 (m, 18H).

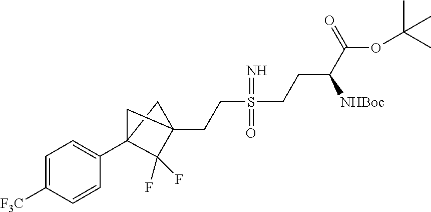 [SMILES: CC(C)(C)OC(=O)N[C@@H](CCS(=N)(=O)CCC12CC(c3ccc(C(F)(F)F)cc3)(C1)C2(F)F)C(=O)OC(C)(C)C]

To a solution of tert-butyl N-(tert-butoxycarbonyl)-S-(2-(2,2-difluoro-3-(4-(trifluoromethyl)phenyl)bicyclo[1.1.1]pentan-1-yl)ethyl)-L-homocysteinate (158 mg, 279.33 μmol, 1 eq) in i-PrOH (5 mL) was added PhI(OAc)₂ (360 mg, 1.12 mmol, 4 eq) and ammonium carbamate (174 mg, 2.23 mmol, 8 eq). The mixture was stirred at 20° C. for 3 h. The reaction mixture was concentrated under reduced pressure. The residue was diluted with H₂O (10 mL) and extracted with DCM (10 mL×3). The combined organic phase was washed with brine (5 mL), dried with anhydrous Na₂SO₄, filtered, and concentrated under reduced pressure. The residue was purified by prep-TLC (SiO₂, petroleum ether/EtOAc=0:1) to give tert-butyl (2S)-2-((tert-butoxycarbonyl)amino)-4-(2-(2,2-difluoro-3-(4-(trifluoromethyl)phenyl)bicyclo[1.1.1]pentan-1-yl)ethylsulfonimidoyl)butanoate (118 mg, 198 μmol, 70.8% yield) as a yellow oil. ¹H NMR (400 MHz, CDCl₃-d) δ 7.62 (d, 2H), 7.38 (d, 2H), 5.28-5.20 (m, 1H), 4.37-4.26 (m, 1H), 3.29-3.01 (m, 4H), 2.48-2.38 (m, 1H), 2.35-2.28 (m, 4H), 2.22-2.11 (m, 1H), 1.81 (br t, 2H), 1.48 (d, 18H).

A solution of tert-butyl (2S)-2-((tert-butoxycarbonyl)amino)-4-(2-(2,2-difluoro-3-(4-(trifluoromethyl)phenyl)bicyclo[1.1.1]pentan-1-yl)ethylsulfonimidoyl)butanoate (118 mg, 198 μmol, 1 eq) in HCl/dioxane (6M, 2 mL) was stirred at 25° C. for 12 h. The reaction mixture was concentrated under reduced pressure. The residue was purified by prep-HPLC (column: Phenomenex Luna C18 (75×30 mm, 3 μm); mobile phase: [H₂O (0.2% FA)-MeCN]; gradient: 10-50% B over 8.0 min) to give (2S)-2-amino-4-(2-(2,2-difluoro-3-(4-(trifluoromethyl)phenyl)bicyclo[1.1.1]pentan-1-yl)ethylsulfonimidoyl)butanoic acid (43.2 mg, 95.7 μmol, 48.4% yield) as a white solid. LCMS: Rt=2.122 min., (ES⁺) m/z (M+H)⁺=411.2, HPLC Conditions: C. ¹H NMR (400 MHz, MeOD-d₄) δ 7.69 (d, 2H), 7.50 (d, 2H), 3.81-3.70 (m, 1H), 3.53-3.36 (m, 3H), 3.28 (br s, 1H), 2.46-2.22 (m, 6H), 1.93 (br t, 2H).

### Exemplary Embodiment 1aa47

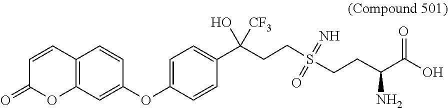 [SMILES: N=S(=O)(CC[C@H](N)C(=O)O)CCC(O)(c1ccc(Oc2ccc3ccc(=O)oc3c2)cc1)C(F)(F)F]

### (2S)-2-amino-4-(4, 4, 4-trifluoro-3-hydroxy-3-(4-((2-oxo-2H-chromen-7-yl) oxy) phenyl) butylsulfonimidoyl) butanoic acid

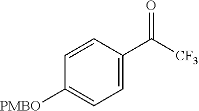

To a mixture of 2,2,2-trifluoro-1-(4-hydroxyphenyl)ethan-1-one (2.5 g, 13.2 mmol, 1 eq) and K₂CO₃ (1.82 g, 13.2 mmol, 1 eq) in DMF (30 mL) was added 1-(chloromethyl)-4-methoxybenzene (2.27 g, 14.5 mmol, 1.96 mL, 1.1 eq), and then the mixture was degassed and purged 3 times with N₂, then the mixture was stirred at 60° C. for 16 h under N₂ atmosphere. The mixture was poured into H₂O (50 mL) and extracted with EtOAc (50 mL×3). The combined organic layers were dried over Na₂SO₄, filtered, and concentrated under reduced pressure. The residue was purified by column chromatography (SiO₂, petroleum ether/EtOAc=92:8) to give 2, 2, 2-trifluoro-1-(4-((4-methoxybenzyl) oxy) phenyl) ethan-1-one (3.96 g, 12.8 mmol, 97.1% yield) as a white solid. ¹H NMR (400 MHz, CDCl₃-d) δ 8.06 (d, 2H), 7.39-7.34 (m, 2H), 7.10-7.05 (m, 2H), 6.95 (d, 2H), 5.11 (s, 2H), 3.84 (s, 3H).

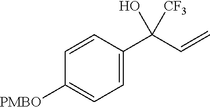

To a solution of 2, 2, 2-trifluoro-1-(4-((4-methoxybenzyl) oxy) phenyl) ethan-1-one (1.00 g, 3.22 mmol, 1 eq) in THE (10 mL) was added bromo(vinyl)magnesium (1 M, 4.83 mL, 1.5 eq) at 0° C. The mixture was stirred at 0° C. for 0.5 h under N₂. The mixture was poured into H₂O (30 mL) and extracted with EtOAc (30 mL×3). The combined organic layers were dried over Na₂SO₄, filtered, and concentrated under reduced pressure. The residue was purified by column chromatography (SiO₂, DCM/MeOH=10:1) to give 1, 1, 1-trifluoro-2-(4-((4-methoxybenzyl) oxy) phenyl) but-3-en-2-ol (1.03 g, 3.04 mmol, 94.4% yield) as a white solid. ¹H NMR (400 MHz, CDCl₃-d) δ 7.56-7.48 (m, 2H), 7.42-7.34 (m, 2H), 7.02-6.97 (m, 2H), 6.95-6.92 (m, 2H), 6.52-6.34 (m, 1H), 5.69-5.46 (m, 2H), 5.09-4.93 (m, 2H), 3.91-3.77 (m, 3H), 2.49 (s, 1H).

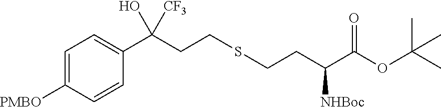

A mixture of 1, 1, 1-trifluoro-2-(4-((4-methoxybenzyl) oxy) phenyl) but-3-en-2-ol (1.02 g, 3.01 mmol, 1 eq), tert-butyl (tert-butoxycarbonyl)-L-homocysteinate (1.32 g, 4.52 mmol, 1.5 eq) and AIBN (84.16 mg, 512.53 μmol, 0.17 eq) in MeOH (0.3 mL) and H₂O (0.1 mL) was degassed and purged 3 times with Ar, and then the mixture was stirred at 60° C. for 16 h under N₂ atmosphere. The mixture was poured into H₂O (20 mL) and extracted with EtOAc (20 mL×3). The combined organic layers were dried over Na₂SO₄, filtered, and concentrated under reduced pressure. The residue was purified by column chromatography (SiO₂, DCM/MeOH=10:1) to give tert-butyl N-(tert-butoxycarbonyl)-S-(4, 4, 4-trifluoro-3-hydroxy-3-(4-((4-methoxybenzyl) oxy) phenyl) butyl)-L-homocysteinate (1.15 g, 1.83 mmol, 60.6% yield) as a white solid. ¹H NMR (400 MHz, CDCl₃-d) δ 7.53-7.48 (m, 2H), 7.39-7.33 (m, 2H), 7.01-6.97 (m, 2H), 6.95-6.90 (m, 2H), 6.42 (dd, 1H), 5.61 (d, 1H), 5.51 (d, 1H), 5.14-5.07 (m, 1H), 5.00 (s, 2H), 4.33-4.23 (m, 1H), 3.83 (s, 3H), 2.76-2.64 (m, 2H), 2.27-2.14 (m, 1H), 2.04-1.95 (m, 1H), 1.49 (s, 8H), 1.46 (s, 8H).

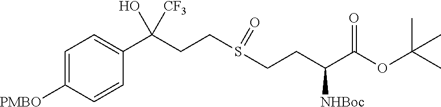

To a solution of tert-butyl N-(tert-butoxycarbonyl)-S-(4, 4, 4-trifluoro-3-hydroxy-3-(4-((4-methoxybenzyl) oxy) phenyl) butyl)-L-homocysteinate (1.1 g, 1.75 mmol, 1 eq) in DCM (1 mL) was added m-CPBA (337 mg, 1.66 mmol, 85% purity, 0.95 eq) at 0° C. The mixture was stirred at 20° C. for 1 h. The mixture was poured into H₂O (20 mL) and extracted with EtOAc (20 mL×3). The combined organic layers were dried over Na₂SO₄, filtered, and concentrated under reduced pressure. The residue was purified by column chromatography (SiO₂, DCM/MeOH=10:1) to give tert-butyl (2S)-2-((tert-butoxycarbonyl) amino)-4-((4, 4, 4-trifluoro-3-hydroxyl-3-(4-((4-methoxybenzyl) oxy) phenyl) butyl) sulfinyl) butanoate (1.1 g, 1.70 mmol, 97.5% yield) as a colorless oil.

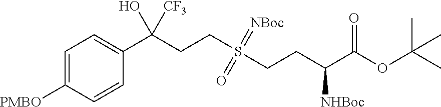

To a solution of tert-butyl (2S)-2-((tert-butoxycarbonyl) amino)-4-((4, 4, 4-trifluoro-3-hydroxyl-3-(4-((4-methoxybenzyl) oxy) phenyl) butyl) sulfinyl) butanoate (1.1 g, 1.70 mmol, 1 eq) in DCM (15 mL) was added tert-butyl carbamate (399 mg, 3.41 mmol, 2 eq), PhI(OAc)₂ (1.10 g, 3.41 mmol, 2 eq), MgO (275 mg, 6.81 mmol, 76.7 μL, 4 eq) and diacetoxyrhodium (18.8 mg, 85.2 μmol, 0.05 eq). The mixture was stirred at 40° C. for 16 h. The mixture was poured into H₂O (50 mL) and extracted with EtOAc (50 mL×3). The combined organic layers were dried over Na₂SO₄, filtered, and concentrated under reduced pressure. The residue was purified by column chromatography (SiO₂, petroleum ether/EtOAc=91:9) to give tert-butyl (2S)-4-(N-(tert-butoxycarbonyl)-4, 4, 4-trifluoro-3-hydroxy-3-(4-((4-methoxybenzyl) oxy) phenyl) butylsulfonimidoyl)-2-((tert-butoxycarbonyl) amino) butanoate (1.08 g, 1.42 mmol, 83.3% yield) as a yellow oil.

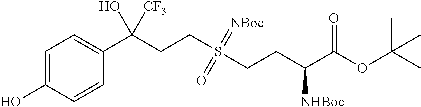

To a solution of tert-butyl (2S)-4-(N-(tert-butoxycarbonyl)-4, 4, 4-trifluoro-3-hydroxy-3-(4-((4-methoxybenzyl) oxy) phenyl) butylsulfonimidoyl)-2-((tert-butoxycarbonyl) amino) butanoate (1.08 g, 1.42 mmol, 1 eq) in EtOH (15 mL) was added Pd/C (267 mg, 2.13 mmol, 85% purity, 1.5 eq). The mixture was stirred at 20° C. for 16 h. The reaction mixture was concentrated under reduced pressure to give tert-butyl (2S)-4-(N-(tert-butoxycarbonyl)-4, 4, 4-trifluoro-3-hydroxy-3-(4-hydroxyphenyl) butylsulfonimidoyl)-2-((tert-butoxycarbonyl) amino) butanoate (800 mg, 1.25 mmol, 88.0% yield) as a colorless oil. ¹H NMR (400 MHz, CDCl₃-d) δ 7.43 (br d, 2H), 6.90-6.85 (m, 2H), 5.38-5.25 (m, 1H), 5.22-5.10 (m, 1H), 4.47-4.29 (m, 2H), 3.47-3.31 (m, 2H), 3.25-3.07 (m, 1H), 2.84-2.46 (m, 2H), 2.37-2.20 (m, 1H), 2.13-2.06 (m, 1H), 1.48-1.46 (m, 27H).

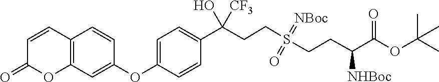

To a solution of tert-butyl (2S)-4-(N-(tert-butoxycarbonyl)-4, 4, 4-trifluoro-3-hydroxy-3-(4-hydroxyphenyl) butylsulfonimidoyl)-2-((tert-butoxycarbonyl) amino) butanoate (50 mg, 78.0 μmol, 1 eq) and 7-bromo-2H-chromen-2-one (35.1 mg, 156 μmol, 2 eq) in DMSO (2 mL) was added (Bu₄NCuI)₂ (17.4 mg, 15.6 μmol, 0.2 eq), DMEDA (8.74 mg, 31.2 μmol, 10.6 μL, 0.4 eq) and Cs₂CO₃ (50.8 mg, 156 μmol, 2 eq). The mixture was stirred at 120° C. for 12 h. The reaction mixture was diluted with H₂O 6 mL and extracted with EtOAc (4 mL×3). The combined organic layers were washed with brine (3 mL×3), dried over Na₂SO₄, filtered, and concentrated under reduced pressure. The residue was purified by prep-TLC (SiO₂, petroleum ether/EtOAc=1:2) to give tert-butyl (2S)-4-(N-(tert-butoxycarbonyl)-4, 4, 4-trifluoro-3-hydroxy-3-(4-((2-oxo-2H-chromen-7-yl) oxy) phenyl) butylsulfonimidoyl)-2-((tert-butoxycarbonyl) amino) butanoate (20 mg, crude) as a yellow oil.

A solution of tert-butyl (2S)-4-(N-(tert-butoxycarbonyl)-4, 4, 4-trifluoro-3-hydroxy-3-(4-((2-oxo-2H-chromen-7-yl) oxy) phenyl) butylsulfonimidoyl)-2-((tert-butoxycarbonyl) amino) butanoate (20 mg, 25.4 μmol, 1 eq) in HCl/dioxane (2 mL) was stirred at 30° C. for 3 h. The reaction mixture was concentrated and the residue was purified by prep-HPLC (FA condition; column: Phenomenex Luna C18 (100×30 mm, 3 μm); mobile phase: [H₂O (0.2% FA)-MeCN]; gradient: 5-45% B over 8.0 min) and prep-HPLC (NH₄HCO₃ condition; column: Waters Xbridge BEH C18 (100×30 mm, 10 μm); mobile phase: [H₂O (10 mm NH₄HCO₃)-MeCN]; gradient:15-45% B over 8.0 min) to give (2S)-2-amino-4-(4, 4, 4-trifluoro-3-hydroxy-3-(4-((2-oxo-2H-chromen-7-yl) oxy) phenyl) butylsulfonimidoyl) butanoic acid (1.09 mg, 1.82 μmol, 48.1% yield) as a white solid. LCMS: Rt=1.995 min, (ES⁺) m/z (M+H)⁺=529.1, HPLC Conditions: C. ¹H NMR (400 MHz, MeOD-d₄) δ 7.93 (d, 1H), 7.70 (d, 2H), 7.64 (d, 1H), 7.18 (d, 2H), 7.01 (dd, 1H), 6.91 (s, 1H), 6.34 (d, 1H), 3.66 (br d, 1H), 3.24-3.16 (m, 1H), 2.85-2.69 (m, 2H), 2.64-2.51 (m, 1H), 2.33-2.19 (m, 2H).

### Exemplary Embodiment 1aa48

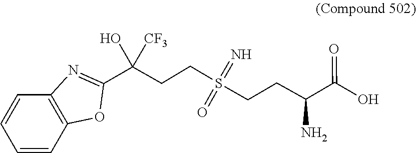 [SMILES: N=S(=O)(CC[C@H](N)C(=O)O)CCC(O)(c1nc2ccccc2o1)C(F)(F)F]

### (2S)-2-amino-4-[[3-(1,3-benzoxazol-2-yl)-4,4,4-trifluoro-3-hydroxy-butyl]sulfonimidoyl]butanoic acid

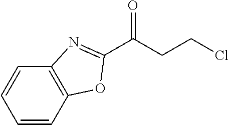 [SMILES: O=C(CCCl)c1nc2ccccc2o1]

1,3-Benzoxazole (500 mg, 4.20 mmol, 1 eq) was dissolved in anhydrous THE (18 mL) and cooled to −65° C. under N₂ atmosphere. n-BuLi (2.5 M, 1.85 mL, 1.1 eq) was added dropwise and the reaction mixture was stirred 20 min at −65° C. under N₂ atmosphere. Dichlorozinc (0.7 M, 12.0 mL, 2 eq) was added to the reaction mixture dropwise at −65° C. under N₂ atmosphere. After the addition was complete, the reaction was warmed to 0° C. and stirred for 45 min at 0° C. under N₂ atmosphere. CuI (799.40 mg, 4.20 mmol, 1 eq) was added and the reaction was stirred for an additional 10 min at 0° C. under N₂ atmosphere. 3-Chloropropanoyl chloride (1.07 g, 8.39 mmol, 806.27 μL, 2 eq) was added quickly to the reaction mixture and stirring was continued at 0° C. for 30 min under N₂ atmosphere. The reaction mixture was diluted with EtOAc (100 mL). The organic layer was washed with 1:1 v/v NH₄OH/H₂O (250 mL), H₂O (220 mL) and saturated aqueous NaCl (210 mL), followed by concentration under reduced pressure to give 1-(1,3-benzoxazol-2-yl)-3-chloro-propan-1-one (1.2 g, crude) was obtained as a yellow oil. The crude product was used in the next step directly without purification.

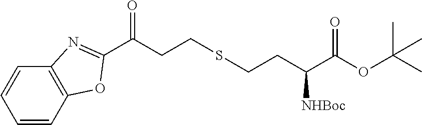 [SMILES: CC(C)(C)OC(=O)N[C@@H](CCSCCC(=O)c1nc2ccccc2o1)C(=O)OC(C)(C)C]

To a solution of 1-(1,3-benzoxazol-2-yl)-3-chloro-propan-1-one (0.7 g, 3.34 mmol, 1 eq) and tert-butyl (2S)-2-(tert-butoxycarbonylamino)-4-sulfanylbutanoate (876 mg, 3.01 mmol, 0.9 eq) in DMF (10 mL) was added K₂CO₃ (1.38 g, 10.02 mmol, 3 eq) and KI (1.11 g, 6.68 mmol, 2 eq). The mixture was stirred at 40° C. for 14 h under Ar atmosphere. The mixture was poured into water (10 mL) and extracted with EtOAc (15 mL×3). The combined organic layers were dried over Na₂SO₄, filtered, and concentrated under reduced pressure. The residue was purified by column chromatography (SiO₂, petroleum ether/EtOAc=90:10). The residue was purified by prep-HPLC (column: Phenomenex Luna C18 (200×40 mm, 10 μm); mobile phase: [water (0.2% FA)-MeCN]; gradient: 60-90% B over 8.0 min) to give tert-butyl (2S)-4-[3-(1,3-benzoxazol-2-yl)-3-oxo-propyl]sulfanyl-2-(tert-butoxycarbonylamino)butanoate (0.4 g, 861 μmol, 25.8% yield) as a white solid. ¹H NMR (400 MHz, CDCl₃-d) δ 7.91 (d, 1H), 7.67 (d, 1H), 7.58-7.52 (m, 1H), 7.51-7.45 (m, 1H), 5.14 (br d, 1H), 4.29 (brd, 1H), 3.58-3.48 (m, 2H), 3.00 (t, 2H), 2.69-2.60 (m, 2H), 2.18-2.03 (m, 1H), 1.98-1.84 (m, 1H), 1.49-1.43 (m, 18H).

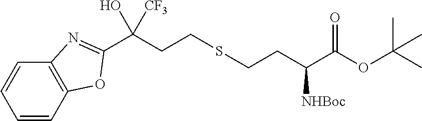 [SMILES: CC(C)(C)OC(=O)N[C@@H](CCSCCC(O)(c1nc2ccccc2o1)C(F)(F)F)C(=O)OC(C)(C)C]

To a solution of tert-butyl (2S)-4-[3-(1,3-benzoxazol-2-yl)-3-oxo-propyl]sulfanyl-2-(tert-butoxycarbonylamino)butanoate (150 mg, 323 μmol, 1 eq) in THE (2 mL) was added TMSCF₃ (138 mg, 969 μmol, 3 eq) and TBAF (1 M, 32.3 μL, 0.1 eq) at 0° C. The mixture was stirred at 0-15° C. for 2 h under N₂ atmosphere. Then TBAF (1 M, 969 μL, 3 eq) was added, and the mixture was stirred at 15° C. for 1 h under N₂ atmosphere. The mixture was poured into water (10 mL) and extracted with EtOAc (15 mL×3). The combined organic layers were dried over Na₂SO₄, filtered and concentrated under reduced pressure and the residue was purified by column chromatography (SiO₂, Petroleum ether/EtOAc=2:1) to give tert-butyl (2S)-4-[3-(1,3-benzoxazol-2-yl)-4,4,4-trifluoro-3-hydroxy-butyl]sulfanyl-2-(tertbutoxycarbonylamino) butanoate (70 mg, 131 μmol, 40.6% yield) as a white yellow oil. ¹H NMR (400 MHz, CDCl₃-d) δ 7.82-7.75 (m, 1H), 7.63 (td, 1H), 7.49-7.38 (m, 2H), 5.11 (br d, 1H), 4.25 (br s, 1H), 2.80-2.68 (m, 1H), 2.67-2.43 (m, 5H), 2.40-2.30 (m, 1H), 2.00 (dt, 1H), 1.88-1.79 (m, 1H), 1.46-1.44 (m, 18H).

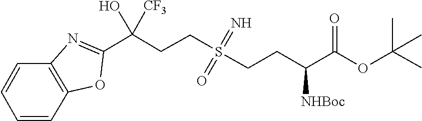 [SMILES: CC(C)(C)OC(=O)N[C@@H](CCS(=N)(=O)CCC(O)(c1nc2ccccc2o1)C(F)(F)F)C(=O)OC(C)(C)C]

To a solution of tert-butyl (2S)-4-[3-(1,3-benzoxazol-2-yl)-4,4,4-trifluoro-3-hydroxy-butyl]sulfanyl-2-(tertbutoxycarbonylamino) butanoate (70 mg, 130.94 μmol, 1 eq) in i-PrOH (3 mL) was added ammonium carbamate (81.78 mg, 1.05 mmol, 8 eq) and PhI(OAc)₂ (169 mg, 524 μmol, 4 eq). The mixture was stirred at 30° C. for 16 h under N₂ atmosphere. The mixture was poured into water (10 mL) and extracted with EtOAc (15 mL×3). The combined organic layers were dried over Na₂SO₄, filtered, and concentrated under reduced pressure. The residue was purified by column chromatography (SiO₂, Petroleum ether: Ethyl acetate=1:1) to give tert-butyl (2S)-4-[[3-(1,3-benzoxazol-2-yl)-4,4,4-trifluoro-3-hydroxy-butyl]sulfonimidoyl]-2-(tertbutoxycarbonylamino) butanoate (35 mg, 61.88 μmol, 47.26% yield) as a yellow oil.

¹H NMR (400 MHz, CDCl₃-d) δ 8.03 (s, 1H), 7.79 (br d, 1H), 7.67-7.60 (m, 1H), 7.50-7.38 (m, 2H), 5.29-5.13 (m, 1H), 4.35-4.19 (m, 1H), 3.35 (br dd, 1H), 3.29-2.99 (m, 4H), 2.81-2.70 (m, 1H), 2.49-2.27 (m, 1H), 2.19-1.95 (m, 1H), 1.49-1.41 (m, 18H).

A mixture of tert-butyl (2S)-4-[[3-(1,3-benzoxazol-2-yl)-4,4,4-trifluoro-3-hydroxy-butyl]sulfonimidoyl]-2-((55 mg, 97.24 μmol, 1 eq) in HCl/dioxane (4 M, 10 mL) was stirred at 25° C. for 6 h. The reaction mixture was concentrated under reduced pressure to give a residue. The residue was purified by prep-HPLC (column: Waters Xbridge Prep OBD C18 (150×40 mm, 10 μm); mobile phase: [NH₄HCO₃-MeCN]; gradient: 5%-30% B over 8.0 min) to give (2S)-2-amino-4-[[3-(1,3-benzoxazol-2-yl)-4,4,4-trifluoro-3-hydroxy-butyl]sulfonimidoyl]butanoic acid (3 mg, 6.89 μmol, 18.55% yield, FA) as a white solid. LCMS: Rt=1.612 min, (ES⁺) m/z (M+H)⁺=410.1, HPLC Conditions: C. ¹H NMR (400 MHz, D₂O) δ 7.79 (d, 1H), 7.71 (d, 1H), 7.56-7.44 (m, 2H), 3.87-3.75 (m, 1H), 3.53-3.40 (m, 2H), 3.40-3.31 (m, 1H), 3.28-3.17 (m, 1H), 2.96-2.84 (m, 1H), 2.79-2.64 (m, 1H), 2.36-2.22 (m, 2H).

### Exemplary Embodiment 1aa49

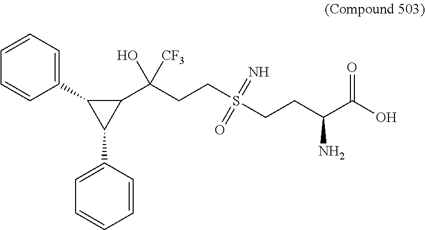 [SMILES: N=S(=O)(CC[C@H](N)C(=O)O)CCC(O)(C1[C@@H](c2ccccc2)[C@H]1c1ccccc1)C(F)(F)F]

### (2S)-2-amino-4-[[3-[(2S, 3R)-2, 3-diphenylcyclopropyl]-4, 4, 4-trifluoro-3-hydroxy-butyl]sulfonimidoyl]butanoic acid

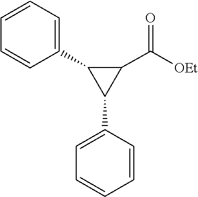 [SMILES: CCOC(=O)C1[C@@H](c2ccccc2)[C@H]1c1ccccc1]

A mixture of (Z)-1,2-diphenylethene (2 g, 11.1 mmol, 6.94 mL, 1 eq), ethyl 2-diazoacetate (3.80 g, 33.2 mmol, 3.50 mL, 3 eq), CuSO₄ (354 mg, 2.22 mmol, 340 μL, 0.2 eq) and ethyl 2-diazoacetate (3.80 g, 33.2 mmol, 3.50 mL, 3 eq) in toluene (14 mL) was degassed and purged with N₂ for 3 times, and then the mixture was stirred at 90° C. for 16 h under N₂ atmosphere. The reaction mixture was filtered and concentrated under reduced pressure. The residue was purified by column chromatography (SiO₂, petroleum ether/EtOAc=94:6) to give ethyl (2S, 3R)-2,3-diphenylcyclopropanecarboxylate (908 mg, 3.41 mmol, 30.7% yield) as a white solid. ¹H NMR (400 MHz, CDCl₃-d) δ 7.17-7.10 (m, 6H), 6.98-6.93 (m, 4H), 4.27-4.23 (m, 2H), 3.07 (d, 2H), 2.55 (t, 1H), 1.35-1.32 (m, 3H).

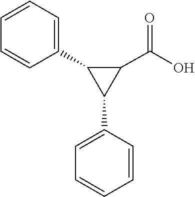 [SMILES: O=C(O)C1[C@@H](c2ccccc2)[C@H]1c1ccccc1]

A mixture of ethyl (2S,3R)-2,3-diphenylcyclopropanecarboxylate (908 mg, 3.41 mmol, 1 eq), NaOH (272 mg, 6.82 mmol, 2 eq) in THE (3.5 mL), H₂O (3.5 mL) and EtOH (3.5 mL) was degassed and purged 3 times with N₂, and then the mixture was stirred at 25° C. for 23 h under N₂ atmosphere. The mixture was diluted with H₂O (10 mL) and extracted with ethyl acetate (10 mL×3). The water layer was adjusted to pH=6 with 1M HCl and extracted with EtOAc (20 mL×2). The combined organic phase was washed with brine (10 mL), dried over Na₂SO₄, filtered, and concentrated under reduced pressure to obtain (2S,3R)-2, 3-diphenylcyclopropanecarboxylic acid (541 mg, crude) as a white oil. ¹H NMR (400 MHz, CDCl₃-d) δ 7.17-7.11 (m, 6H), 6.96 (dd, 4H), 3.14 (d, 2H), 2.57 (t, 1H). The residue was used for the next step directly.

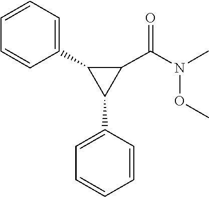 [SMILES: CON(C)C(=O)C1[C@@H](c2ccccc2)[C@H]1c1ccccc1]

A mixture of (2S,3R)-2, 3-diphenylcyclopropanecarboxylic acid (541 mg, 2.27 mmol, 1 eq), di(imidazol-1-yl)methanone (405 mg, 2.50 mmol, 1.1 eq) in DCM (10 mL) was degassed and purged 3 times with N₂, and then the mixture was stirred at 0° C. for 0.5 h. N,O-Dimethylhydroxylamine hydrochloride (265 mg, 2.73 mmol, 1.2 eq) was added and the solution was stirred for 0.5 h at 0° C. and stirred at 15° C. for 15 h under N₂ atmosphere. The reaction mixture was quenched by addition of EtOAc (5 mL) and H₂O (5 mL) at 25° C., and then extracted with EtOAc (5 mL×2). The combined organic layers were washed with brine (5 mL×2). The combined organic layers were dried over Na₂SO₄, filtered, and concentrated under reduced pressure. The residue was purified by prep-TLC (SiO₂, petroleum ether/EtOAc=1:1, R_{f}=0.5) to give (2S,3R)—N-methoxy-N-methyl-2,3-diphenyl-cyclopropanecarboxamide (511 mg, 1.82 mmol, 80.0% yield) as a white solid. ¹H NMR (400 MHz, CDCl₃-d) δ 7.17-7.10 (m, 6H), 6.98 (dd, 4H), 3.81 (s, 3H), 3.32 (s, 3H), 3.09 (s, 3H).

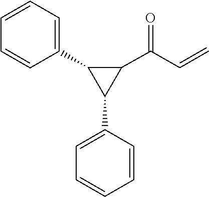 [SMILES: C=CC(=O)C1[C@@H](c2ccccc2)[C@H]1c1ccccc1]

To a solution of (2S,3R)—N-methoxy-N-methyl-2,3-diphenyl-cyclopropanecarboxamide (400 mg, 1.42 mmol, 1 eq) in THE (5 mL) was added bromo(vinyl)magnesium (1 M, 3.55 mL, 2.5 eq). The mixture was stirred at −65° C. for 10 min under N₂. Then, tert-butyl (2S)-2-(tert-butoxycarbonylamino)-4-sulfanyl-butanoate (373 mg, 1.28 mmol, 0.9 eq) and TEA (575 mg, 5.69 mmol, 792 μL, 4 eq) in THF (3 mL) were added. The mixture was stirred at 0° C. for 16 h under N₂. The mixture was poured into H₂O (40 mL) and extracted with EtOAc (40 mL×3). The combined organic layers were dried over Na₂SO₄, filtered, and concentrated under reduced pressure. The residue was purified by prep-TLC (SiO₂, petroleum ether/EtOAc=92:8) to give tert-butyl N-(tert-butoxycarbonyl)-S-(3-((2R, 3S)-2, 3-diphenylcyclopropyl)-3-oxopropyl)-L-homocysteinate (270 mg, 500 μmol, 35.2% yield) as a colorless oil. ¹H NMR (400 MHz, CDCl₃-d) δ 7.18-7.10 (m, 6H), 6.96-6.90 (m, 4H), 5.19-5.05 (m, 1H), 4.33-4.22 (m, 1H), 3.13 (d, 2H), 3.10-3.04 (m, 2H), 2.89-2.85 (m, 2H), 2.85-2.79 (m, 2H), 2.63-2.56 (m, 2H), 2.17-2.06 (m, 1H), 1.47 (s, 9H), 1.45 (s, 9H).

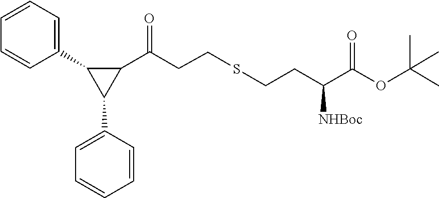 [SMILES: CC(C)(C)OC(=O)N[C@@H](CCSCCC(=O)C1[C@@H](c2ccccc2)[C@H]1c1ccccc1)C(=O)OC(C)(C)C]

To a solution of tert-butyl N-(tert-butoxycarbonyl)-S-(3-((2R, 3S)-2, 3-diphenylcyclopropyl)-3-oxopropyl)-L-homocysteinate (270 mg, 500 μmol, 1 eq) in THF (4 mL) was added TMSCF₃ (711 mg, 5.00 mmol, 10 eq) and then added TBAF (1 M, 100 μL, 0.2 eq) at 0° C. The mixture was stirred at 25° C. for 0.5 h under N₂. Then TBAF (1 M, 1.50 mL, 3 eq) was added and the mixture was stirred at 25° C. for 0.5 h under N₂. The mixture was poured into H₂O (40 mL) and extracted with EtOAc (40 mL×3). The combined organic layers were dried over Na₂SO₄, filtered, and concentrated under reduced pressure. The residue was purified by prep-TLC (SiO₂, DCM/MeOH=10:1) to give tert-butyl N-(tert-butoxycarbonyl)-S-(3-((2R, 3S)-2, 3-diphenylcyclopropyl)-4, 4, 4-trifluoro-3-hydroxybutyl)-L-homocysteinate (51 mg, 83.6 μmol, 16.7% yield) as a yellow oil. ¹H NMR (400 MHz, CDCl₃-d) δ 7.17-7.06 (m, 6H), 7.05-6.97 (m, 2H), 6.91-6.83 (m, 2H), 5.19-5.07 (m, 1H), 4.35 (br d, 1H), 2.94-2.80 (m, 2H), 2.78-2.71 (m, 1H), 2.68-2.51 (m, 3H), 2.33-2.14 (m, 2H), 2.03-1.95 (m, 1H), 1.93-1.76 (m, 2H), 1.46 (br d, 18H).

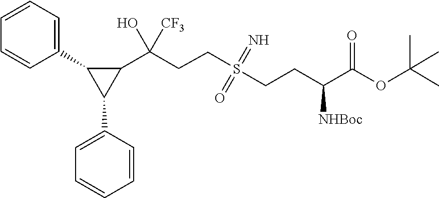 [SMILES: CC(C)(C)OC(=O)N[C@@H](CCS(=N)(=O)CCC(O)(C1[C@@H](c2ccccc2)[C@H]1c1ccccc1)C(F)(F)F)C(=O)OC(C)(C)C]

To a solution of tert-butyl N-(tert-butoxycarbonyl)-S-(3-((2R, 3S)-2, 3-diphenylcyclopropyl)-4, 4, 4-trifluoro-3-hydroxybutyl)-L-homocysteinate (51 mg, 83.6 μmol, 1 eq) in i-PrOH (1 mL) was added PhI(OAc)₂ (108 mg, 335 μmol, 4 eq) and ammonium carbamate (52.2 mg, 669 μmol, 8 eq). The mixture was stirred at 25° C. for 16 h. The mixture was poured into H₂O (30 mL) and extracted with EtOAc (30 mL×3). The combined organic layers were dried over Na₂SO₄, filtered, and concentrated under reduced pressure. The residue was purified by prep-TLC (SiO₂, DCM/MeOH=10:1) to give tert-butyl (2S)-2-((tert-butoxycarbonyl)amino)-4-(3-((2R,3S)-2,3-diphenylcyclopropyl)-4,4,4-trifluoro-3-hydroxybutylsulfonimidoyl) butanoate (30 mg, 46.8 μmol, 56.0% yield) as a colorless oil.

A solution of tert-butyl (2S)-2-((tert-butoxycarbonyl)amino)-4-(3-((2R,3S)-2,3-diphenylcyclopropyl)-4,4,4-trifluoro-3-hydroxybutylsulfonimidoyl) butanoate (30 mg, 46.8 μmol, 1 eq) in HCl/dioxane (4 M, 10 mL) was stirred at 25° C. for 16 h. The mixture was concentrated and the residue was purified by prep-HPLC: (column: Waters Xbridge BEH C18 (100×30 mm, 5 μm); mobile phase: [H₂O (10 mM NH₄HCO₃)-MeCN]; gradient: 17-42% B over 8.0 min) to give (2S)-2-amino-4-(3-((2R,3S)-2,3-diphenylcyclopropyl)-4,4,4-trifluoro-3-hydroxybutylsulfonimidoyl) butanoic acid (2.11 mg, 4.35 μmol, 9.30% yield) as a white solid. LCMS: Rt=2.101 min, (ES⁺) m/z (M+H)⁺=485.2, HPLC Conditions: C. ¹H NMR (400 MHz, MeOD-d₄) δ 7.14-7.01 (m, 6H), 7.01-6.95 (m, 4H), 3.69-3.58 (m, 1H), 3.44-3.35 (m, 1H), 3.29-3.13 (m, 3H), 2.81 (dd, 1H), 2.76 (s, 1H), 2.51-2.40 (m, 2H), 2.29-2.18 (m, 2H), 2.10-2.01 (m, 1H).

### Exemplary Embodiment 1aa50

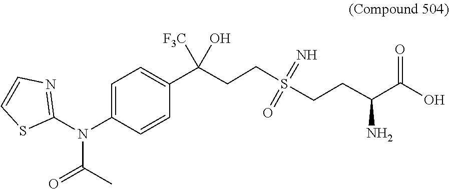 [SMILES: CC(=O)N(c1ccc(C(O)(CCS(=N)(=O)CC[C@H](N)C(=O)O)C(F)(F)F)cc1)c1nccs1]

### (2S)-2-amino-4-(4,4,4-trifluoro-3-hydroxy-3-(4-(N-(thiazol-2 yl)acetamido)phenyl)butylsulfonimidoyl)butanoic acid

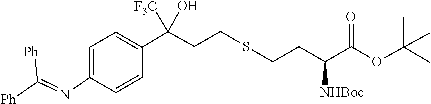 [SMILES: CC(C)(C)OC(=O)N[C@@H](CCSCCC(O)(c1ccc(N=C(c2ccccc2)c2ccccc2)cc1)C(F)(F)F)C(=O)OC(C)(C)C]

To a mixture of diphenylmethanimine (411 mg, 2.27 mmol, 381 μL, 1.3 eq), tert-butyl S-(3-(4-bromophenyl)-4,4,4-trifluoro-3-hydroxybutyl)-N-(tert-butoxycarbonyl)-L-homocysteinate (1.00 g, 1.75 mmol, 1 eq) and Xantphos (60.6 mg, 105 μmol, 0.06 eq) in dioxane (20 mL) was added Cs₂CO₃ (1.71 g, 5.24 mmol, 3 eq) and Pd₂(dba)₃ (80.0 mg, 87.3 μmol, 0.05 eq) under N₂, the resulting mixture was stirred at 100° C. for 16 h. After cooling, the mixture was filtered, the filtrate was quenched with water (20 mL) and extracted with EtOAc (20 mL×2), the combined organic phases were washed brine (20 mL), dried over Na₂SO₄, filtered and the filtrate was concentrated. The residue was purified by silica gel chromatography (petroleum ether/EtOAc=10:1 to 5:1) to give tert-butyl N-(tert-butoxycarbonyl)-S-(3-(4-((diphenylmethylene)amino)phenyl)-4,4,4-trifluoro-3-hydroxybutyl)-L-homocysteinate (0.9 g, 1.34 mmol, 76.58% yield) as yellow solid. ¹H NMR (400 MHz, CDCl₃-d) δ 7.76 (d, 2H), 7.52-7.38 (m, 5H), 7.31 (d, 2H), 7.25-7.21 (m, 1H), 7.10 (d, 2H), 6.78-6.71 (m, 2H), 5.24-5.09 (m, 1H), 4.36-4.22 (m, 1H), 3.90-3.65 (m, 1H), 2.62-2.21 (m, 6H), 2.04-1.77 (m, 2H), 1.50-1.43 (m, 18H).

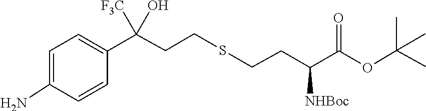 [SMILES: CC(C)(C)OC(=O)N[C@@H](CCSCCC(O)(c1ccc(N)cc1)C(F)(F)F)C(=O)OC(C)(C)C]

A mixture of tert-butyl N-(tert-butoxycarbonyl)-S-(3-(4-((diphenylmethylene)amino)phenyl)-4,4,4-trifluoro-3-hydroxybutyl)-L-homocysteinate (0.9 g, 1.34 mmol, 1 eq), NH₂OH·HCl (186 mg, 2.68 mmol, 2 eq) and NaOAc (274 mg, 3.34 mmol, 2.5 eq) in methanol (10 mL) was stirred at 15° C. for 3 hr. The mixture was concentrated, the residue was added water (10 mL) and extracted with EtOAc (10 mL×2), the combined organic phases were washed with brine (10 mL), dried over Na₂SO₄, filtered and concentrated. The residue was purified by prep-TLC (petroleum ether/EtOAc=2:1) to give tert-butyl S-(3-(4-aminophenyl)-4,4,4-trifluoro-3-hydroxybutyl)-N-(tert-butoxycarbonyl)-L-homocysteinate (0.54 g, 1.06 mmol, 79.08% yield) as a yellow solid. ¹H NMR (400 MHz, CDCl₃-d) δ 7.31 (d, 2H), 6.70 (dd, 2H), 5.23-5.05 (m, 1H), 4.27 (br dd, 1H), 3.75 (br s, 2H), 2.62-2.50 (m, 3H), 2.48 (br d, 3H), 2.35-2.22 (m, 1H), 2.03-1.81 (m, 2H), 1.47-1.46 (m, 18H).

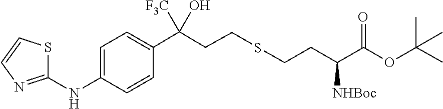 [SMILES: CC(C)(C)OC(=O)N[C@@H](CCSCCC(O)(c1ccc(Nc2nccs2)cc1)C(F)(F)F)C(=O)OC(C)(C)C]

A mixture of tert-butyl S-(3-(4-aminophenyl)-4,4,4-trifluoro-3-hydroxybutyl)-N-(tert-butoxycarbonyl)-L-homocysteinate (0.18 g, 354 μmol, 1 eq), 2-bromothiazole (87.1 mg, 531 μmol, 47.8 μL, 1.5 eq) and p-TsOH (6.09 mg, 35.4 μmol, 0.1 eq) in i-PrOH (5 mL) was heated to 80° C. for 16 hr. The mixture was concentrated, the residue was dissolved in dioxane (2 mL), Boc₂O (85.0 mg, 389 μmol, 89.5 μL, 1.1 eq) and TEA (71.6 mg, 708 μmol, 98.5 μL, 2 eq) in was stirred at 25° C. for 3 hr. The mixture was quenched with water (10 mL) and extracted with DCM (10 mL×2), the combined organic phases were dried over Na₂SO₄, filtered and concentrated. The residue was purified by prep-TLC (petroleum ether/EtOAc=2:1) to give tert-butyl N-(tert-butoxycarbonyl)-S-(4,4,4-trifluoro-3-hydroxy-3-(4-(thiazol-2-ylamino)phenyl)butyl)-L-homocysteinate (0.1 g, 159.37 μmol, 45.03% yield) as a yellow solid. ¹H NMR (400 MHz, CDCl₃-d) δ 7.57-7.50 (m, 2H), 7.42 (dd, 2H), 7.32 (d, 1H), 6.70 (dd, 1H), 5.25-5.08 (m, 1H), 4.38-4.20 (m, 1H), 4.06-3.80 (m, 1H), 2.61-2.31 (m, 6H), 2.10-1.78 (m, 2H), 1.47-1.45 (m, 18H).

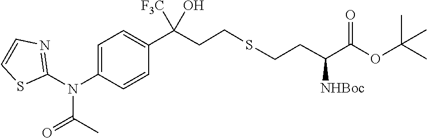 [SMILES: CC(=O)N(c1ccc(C(O)(CCSCC[C@H](NC(=O)OC(C)(C)C)C(=O)OC(C)(C)C)C(F)(F)F)cc1)c1nccs1]

To a solution of tert-butyl N-(tert-butoxycarbonyl)-S-(4,4,4-trifluoro-3-hydroxy-3-(4-(thiazol-2-ylamino)phenyl)butyl)-L-homocysteinate (0.1 g, 169 μmol, 1 eq) and DIPEA (43.7 mg, 338 μmol, 58.9 μL, 2 eq) in DCM (1 mL) was added Ac₂O (20.7 mg, 203 μmol, 19.1 μL, 1.2 eq) at 0° C., the mixture was stirred at 15° C. for 3 hr. The mixture was concentrated, the residue was purified by prep-TLC (petroleum ether/EtOAc=2:1) to give tert-butyl N-(tert-butoxycarbonyl)-S-(4,4,4-trifluoro-3-hydroxy-3-(4-(N-(thiazol-2-yl)acetamido)phenyl)butyl)-L-homocysteinate (0.1 g, crude) as a yellow solid.

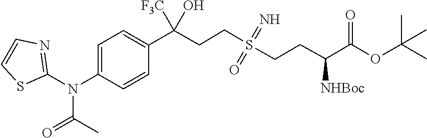 [SMILES: CC(=O)N(c1ccc(C(O)(CCS(=N)(=O)CC[C@H](NC(=O)OC(C)(C)C)C(=O)OC(C)(C)C)C(F)(F)F)cc1)c1nccs1]

A mixture of tert-butyl N-(tert-butoxycarbonyl)-S-(4,4,4-trifluoro-3-hydroxy-3-(4-(N-(thiazol-2-yl)acetamido)phenyl)butyl)-L-homocysteinate (0.1 g, 158 μmol, 1 eq), PIDA (203 mg, 631 μmol, 4 eq) and ammonium carbamate (98.6 mg, 1.26 mmol, 8 eq) in i-PrOH (3 mL) was stirred at 15° C. for 3 h. The mixture was concentrated, the residue was diluted with water (10 mL) and extracted with EtOAc (10 mL×2), the combined organic phases were dried over Na₂SO₄, filtered and concentrated, the residue was purified by prep-TLC (petroleum ether/EtOAc=1:1) to give tert-butyl (2S)-2-((tert-butoxycarbonyl)amino)-4-(4,4,4-trifluoro-3-hydroxy-3-(4-(N-(thiazol-2-yl)acetamido)phenyl)butylsulfonimidoyl)butanoate (25 mg, 37.61 μmol, 23.83% yield) as yellow solid.

A solution of tert-butyl (2S)-2-((tert-butoxycarbonyl)amino)-4-(4,4,4-trifluoro-3-hydroxy-3-(4-(N-(thiazol-2-yl)acetamido)phenyl)butylsulfonimidoyl)butanoate (25 mg, 37.61 μmol, 1 eq) in HCl/dioxane (10 mL) was stirred at 25° C. for 6 h. The mixture was concentrated and the residue was purified by prep-HPLC (column: Phenomenex Gemini-NX C18 (75×30 mm, 3 μm); mobile phase: [H₂O (0.2% FA)-MeCN]; gradient: 10-40% B over 9.0 min) to give (2S)-2-amino-4-(4,4,4-trifluoro-3-hydroxy-3-(4-(N-(thiazol-2 yl)acetamido)phenyl)butylsulfonimidoyl)butanoic acid (5 mg, 8.84 μmol, 23.52% yield, FA) as a white solid. LCMS: Rt=2.093 min, (ES⁺) m/z (M+H)⁺=509.0, HPLC condition: B. ¹H NMR (400 MHz, MeOD-d₄) δ 7.85 (d, 2H), 7.47 (d, 2H), 7.32 (d, 1H), 7.21 (d, 1H), 3.76-3.63 (m, 1H), 3.45-3.34 (m, 1H), 3.29-3.16 (m, 2H), 3.04-2.92 (m, 1H), 2.88-2.71 (m, 1H), 2.65-2.54 (m, 1H), 2.38-2.23 (m, 2H), 2.04 (s, 3H).

### Exemplary Embodiment 1aa51

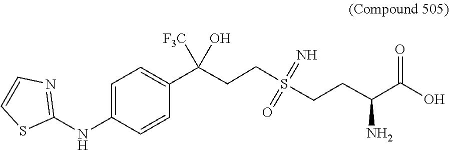 [SMILES: N=S(=O)(CC[C@H](N)C(=O)O)CCC(O)(c1ccc(Nc2nccs2)cc1)C(F)(F)F]

### (2S)-2-amino-4-(4,4,4-trifluoro-3-hydroxy-3-(4-(thiazol-2-ylamino)phenyl)butylsulfonimidoyl)butanoic acid

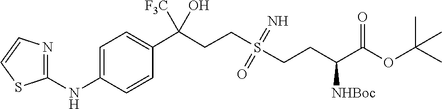 [SMILES: CC(C)(C)OC(=O)N[C@@H](CCS(=N)(=O)CCC(O)(c1ccc(Nc2nccs2)cc1)C(F)(F)F)C(=O)OC(C)(C)C]

A mixture of tert-butyl N-(tert-butoxycarbonyl)-S-(4,4,4-trifluoro-3-hydroxy-3-(4-(thiazol-2-ylamino)phenyl)butyl)-L-homocysteinate (20 mg, 33.8 μmol, 1 eq), PIDA (32.7 mg, 101 μmol, 3 eq) and ammonium carbamate (15.8 mg, 203 μmol, 6 eq) in i-PrOH (2 mL) was stirred at 20° C. for 2 hr. The mixture was concentrated, the residue was diluted with water (10 mL) and extracted with EtOAc (10 mL×2), the combined organic phases were dried over Na₂SO₄, filtered and concentrated, the residue was purified by prep-TLC (petroleum ether/EtOAc=1:1) to give tert-butyl (2S)-2-((tert-butoxycarbonyl)amino)-4-(4,4,4-trifluoro-3-hydroxy-3-(4-(thiazol-2-ylamino)phenyl)butylsulfonimidoyl)butanoate (10 mg, 16.06 μmol, 47.51% yield) as a yellow solid.

A solution of tert-butyl (2S)-2-((tert-butoxycarbonyl)amino)-4-(4,4,4-trifluoro-3-hydroxy-3-(4-(thiazol-2-ylamino)phenyl)butylsulfonimidoyl)butanoate (10 mg, 16.1 μmol, 1 eq) in HCl/dioxane (10 mL) was stirred at 25° C. for 3 h. The mixture was concentrated, the residue was purified by prep-HPLC (column: Phenomenex Gemini-NX C18 (75×30 mm, 3 μm); mobile phase: [H₂O (0.2% FA)-MeCN]; gradient: 5-35% B over 9.0 min) to give (2S)-2-amino-4-(4,4,4-trifluoro-3-hydroxy-3-(4-(thiazol-2-ylamino)phenyl)butylsulfonimidoyl)butanoic acid (2.28 mg, 4.22 μmol, 26.30% yield, FA) as a white solid. LCMS: Rt=2.092 min, (ES⁺) m/z (M+H)⁺=467.0. HPLC condition: B. ¹H NMR (400 MHz, MeOD-d₄) δ 7.53-7.47 (m, 2H), 7.46-7.40 (m, 2H), 7.12 (d, 1H), 6.70 (d, 1H), 3.62-3.52 (m, 2H), 3.18-3.05 (m, 2H), 2.73-2.58 (m, 2H), 2.48-2.37 (m, 1H), 2.25-2.10 (m, 2H).

### Exemplary Embodiment 1aa52

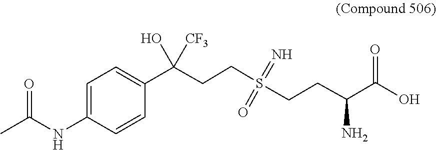 [SMILES: CC(=O)Nc1ccc(C(O)(CCS(=N)(=O)CC[C@H](N)C(=O)O)C(F)(F)F)cc1]

### (2S)-4-(3-(4-acetamidophenyl)-4,4,4-trifluoro-3-hydroxybutylsulfonimidoyl)-2-aminobutanoic acid

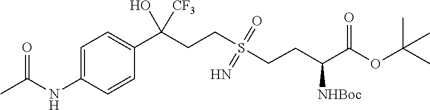 [SMILES: CC(=O)Nc1ccc(C(O)(CCS(=N)(=O)CC[C@H](NC(=O)OC(C)(C)C)C(=O)OC(C)(C)C)C(F)(F)F)cc1]

To a solution of tert-butyl (2S)-4-[3-(4-acetamidophenyl)-4,4,4-trifluoro-3-hydroxy-butyl]sulfanyl-2-(tert-butoxycarbonylamino)butanoate (80 mg, 145 μmol, 1 eq) in i-PrOH (1 mL) was added ammonium carbamate (90.7 mg, 1.16 mmol, 8 eq) and [acetoxy(phenyl)-iodanyl]acetate (187 mg, 581 μmol, 4 eq). The mixture was stirred at 20° C. for 12 h. The reaction mixture was concentrated under reduced pressure to give a residue. The residue was diluted with H₂O (3 mL) and extracted with DCM (3 mL×3). The combined organic phase was washed with brine (1 mL), dried with anhydrous Na₂SO₄, filtered, and concentrated under reduced pressure to give a residue. The residue was purified by prep-TLC (SiO₂, petroleum ether/EtOAc=0:1) to give tert-butyl (2S)-4-(3-(4-acetamidophenyl)-4,4,4-trifluoro-3-hydroxybutylsulfonimidoyl)-2-((tert-butoxycarbonyl)amino)butanoate (30 mg, 51.6 μmol, 35.5% yield) as a yellow oil. ¹H NMR (400 MHz, CDCl₃-d) δ 7.59-7.34 (m, 5H), 4.30-4.11 (m, 1H), 3.58-2.96 (m, 5H), 2.61-2.30 (m, 2H), 2.16-2.03 (m, 4H), 1.70 (br s, 1H), 1.44-1.30 (m, 18H).

A solution of tert-butyl (2S)-4-(3-(4-acetamidophenyl)-4,4,4-trifluoro-3-hydroxybutylsulfonimidoyl)-2-((tert-butoxycarbonyl)amino)butanoate (30 mg, 51.6 μmol, 1 eq) in HCl/dioxane (6M, 4 mL) was stirred at 30° C. for 12 h. The reaction mixture was concentrated under reduced pressure to give a residue. The residue was purified by prep-HPLC (column: Waters Xbridge BEH C18 (100×30 mm, 5 μm); mobile phase: [H₂O (10 mM NH₄HCO₃)-MeCN]; gradient: 1%-24% B over 8.0 min) to give (2S)-4-(3-(4-acetamidophenyl)-4,4,4-trifluoro-3-hydroxybutylsulfonimidoyl)-2-aminobutanoic acid (8.14 mg, 19.13 μmol, 37.10% yield) a white solid. LCMS: Rt=1.703 min, (ES⁺) m/z (M+H)⁺=426.0, HPLC conditions: B. ¹H NMR (400 MHz, D₂O) δ 7.70-7.32 (m, 4H), 3.75 (d, 1H), 3.41-3.17 (m, 3H), 2.94-2.78 (m, 1H), 2.67 (br d, 1H), 2.59-2.43 (m, 1H), 2.26-2.15 (m, 2H), 2.11 (s, 3H).

### Exemplary Embodiment 1aa53

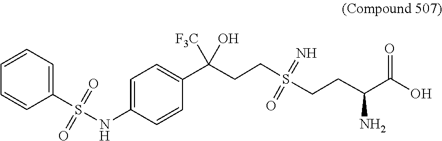 [SMILES: N=S(=O)(CC[C@H](N)C(=O)O)CCC(O)(c1ccc(NS(=O)(=O)c2ccccc2)cc1)C(F)(F)F]

### (2S)-2-amino-4-(4,4,4-trifluoro-3-hydroxy-3-(4-(phenylsulfonamido)phenyl)butylsulfonimidoyl)butanoic acid

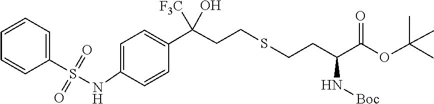 [SMILES: CC(C)(C)OC(=O)N[C@@H](CCSCCC(O)(c1ccc(NS(=O)(=O)c2ccccc2)cc1)C(F)(F)F)C(=O)OC(C)(C)C]

To a solution of tert-butyl S-(3-(4-aminophenyl)-4,4,4-trifluoro-3-hydroxybutyl)-N-(tert-butoxycarbonyl)-L-homocysteinate (100 mg, 197 μmol, 1 eq) in DCM (5 mL) was added Py (77.8 mg, 983 μmol, 79.4 μL, 5 eq) and benzenesulfonyl chloride (38.2 mg, 216 μmol, 27.6 μL, 1.1 eq) at 0° C. The mixture was stirred at 15° C. for 3 h. The reaction mixture was concentrated under reduced pressure to give a residue. The residue was purified by prep-TLC (SiO₂, petroleum ether/EtOAc=2:1) to give tert-butyl N-(tert-butoxycarbonyl)-S-(4,4,4-trifluoro-3-hydroxy-3-(4-(phenylsulfonamido)phenyl)butyl)-L-homocysteinate (115 mg, 176 μmol, 89.6% yield) as a white solid. ¹H NMR (400 MHz, CDCl₃-d) δ 7.71 (d, 2H), 7.54-7.45 (m, 1H), 7.42-7.33 (m, 4H), 7.01 (dd, 2H), 6.49 (br s, 1H), 5.19-5.02 (m, 1H), 4.29-4.14 (m, 1H), 2.58-2.15 (m, 6H), 1.96-1.69 (m, 2H), 1.42-1.36 (m, 18H).

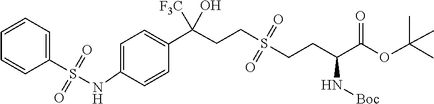 [SMILES: CC(C)(C)OC(=O)N[C@@H](CCS(=O)(=O)CCC(O)(c1ccc(NS(=O)(=O)c2ccccc2)cc1)C(F)(F)F)C(=O)OC(C)(C)C]

A mixture of tert-butyl N-(tert-butoxycarbonyl)-S-(4,4,4-trifluoro-3-hydroxy-3-(4-(phenylsulfonamido)phenyl)butyl)-L-homocysteinate (150 mg, 231 μmol, 1 eq), PhI(OAc)₂ (298 mg, 925 μmol, 4 eq) and ammonium carbamate (144 mg, 1.85 mmol, 8 eq) in i-PrOH (20 mL) was stirred at 15° C. for 48 h. The reaction mixture was concentrated under reduced pressure to remove i-PrOH. The residue was diluted with water (10 mL) and extracted with DCM (25 mL×2). The combined organic layers were dried over Na₂SO₄, filtered, and concentrated under reduced pressure to give a residue. The residue was purified by prep-HPLC (column: Phenomenex Luna C18 (100×40 mm, 5 μm); mobile phase: [H₂O (0.2% FA)-MeCN]; gradient: 25-55% B over 8.0 min) to give tert-butyl (2S)-2-((tert-butoxycarbonyl)amino)-4-(4,4,4-trifluoro-3-hydroxy-3-(4-(phenylsulfonamido)phenyl)butylsulfonimidoyl)butanoate (110 mg, 162 μmol, 70.0% yield) as a colorless oil. ¹H NMR (400 MHz, CDCl₃-d) δ 7.87-7.77 (m, 2H), 7.62-7.54 (m, 1H), 7.48 (dd, 4H), 7.10 (d, 2H), 6.71 (br s, 1H), 5.33-5.11 (m, 1H), 4.33-4.17 (m, 1H), 3.29-2.83 (m, 4H), 2.73-2.54 (m, 2H), 2.44-2.26 (m, 1H), 2.14-2.01 (m, 1H), 1.54-1.41 (m, 18H).

To a solution of tert-butyl (2S)-2-((tert-butoxycarbonyl)amino)-4-(4,4,4-trifluoro-3-hydroxy-3-(4-(phenylsulfonamido)phenyl)butylsulfonimidoyl)butanoate (100 mg, 147 μmol, 1 eq) in dioxane (4 mL) was added HCl/dioxane (4 M, 25 mL). The mixture was stirred at 20° C. for 18 h. The reaction mixture was concentrated under reduced pressure to give a residue. The residue was purified by prep-HPLC (Column: Waters Xbridge Prep OBD C18 (150×40 mm, 10 μm); mobile phase: [H₂O (10 mM NH₄HCO₃)-MeCN]; gradient: 15-45% B over 8.0 min) to give (2S)-2-amino-4-(4,4,4-trifluoro-3-hydroxy-3-(4-(phenylsulfonamido)phenyl)butylsulfonimidoyl)butanoic acid (51.52 mg, 98.0 μmol, 66.6% yield, 99.6% purity) as a white solid. LCMS: Rt=1.830 min, (ES⁺) m/z (M+H)⁺=524.1, HPLC conditions: C. ¹H NMR (400 MHz, MeOD-d₄) δ 7.81 (br d, 2H), 7.64-7.56 (m, 1H), 7.50 (br dd, 4H), 7.17 (br d, 2H), 3.75-3.62 (m, 1H), 3.43-3.35 (m, 1H), 3.29-3.21 (m, 1H), 3.19-3.07 (m, 1H), 2.75-2.60 (m, 2H), 2.58-2.43 (m, 1H), 2.33-2.20 (m, 2H).

### Exemplary Embodiment 1aa54

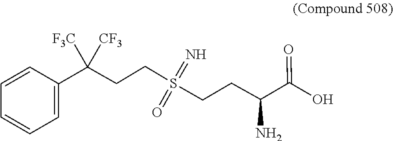 [SMILES: N=S(=O)(CC[C@H](N)C(=O)O)CCC(c1ccccc1)(C(F)(F)F)C(F)(F)F]

### (2S)-2-amino-4-(4,4,4-trifluoro-3-phenyl-3-(trifluoromethyl)butylsulfonimidoyl)butanoic acid

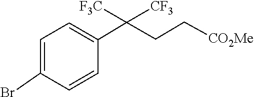 [SMILES: COC(=O)CCC(c1ccc(Br)cc1)(C(F)(F)F)C(F)(F)F]

To a solution of methyl 3-(4-aminophenyl)-4,4,4-trifluoro-3-(trifluoromethyl)butanoate (135 mg, 428 μmol, 1 eq) in MeCN (3 mL) and H₂O (0.3 mL) was added CuBr₂ (115 mg, 514 μmol, 24.1 μL, 1.2 eq) and tert-butyl nitrite (66.2 mg, 642 μmol, 76.4 μL, 1.5 eq). The mixture was stirred at 60° C. for 2 h. The reaction mixture was poured into water (10 mL), extracted with EtOAc (10 mL×2). The combined organic layers were washed with brine (10 mL), dried over Na₂SO₄, filtered, and concentrated under reduced pressure to give a residue. The residue was purified by prep-TLC (SiO₂, petroleum ether/EtOAc=10:1) to give methyl 3-(4-bromophenyl)-4,4,4-trifluoro-3-(trifluoromethyl)butanoate (130 mg, 343 μmol, 80.1% yield) as a yellow oil. ¹H NMR (400 MHz, CDCl₃-d) δ 7.61-7.54 (m, 2H), 7.46 (br d, 2H), 3.64 (s, 3H), 3.38 (s, 2H).

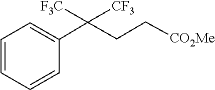 [SMILES: COC(=O)CCC(c1ccccc1)(C(F)(F)F)C(F)(F)F]

To a solution of methyl 3-(4-bromophenyl)-4,4,4-trifluoro-3-(trifluoromethyl)butanoate (190 mg, 501 μmol, 1 eq) in MeOH (1 mL) was added Pd/C (112 mg, 10% purity) under N₂. The mixture was stirred at 25° C. for 1 h under H₂ (15 psi). The reaction mixture was filtered and the filtrate was concentrated under reduced pressure to give methyl 4,4,4-trifluoro-3-phenyl-3-(trifluoromethyl)butanoate (142 mg, 473 μmol, 94.4% yield) as a yellow oil. ¹H NMR (400 MHz, CDCl₃-d) δ 7.58 (br d, 2H), 7.49-7.41 (m, 3H), 3.63 (s, 3H), 3.42 (s, 2H).

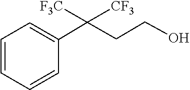 [SMILES: OCCC(c1ccccc1)(C(F)(F)F)C(F)(F)F]

To a solution of methyl 4,4,4-trifluoro-3-phenyl-3-(trifluoromethyl)butanoate (142 mg, 473 μmol, 1 eq) in THE (3 mL) was added LAH (35.9 mg, 946 μmol, 2 eq) at 0° C. The mixture was stirred at 25° C. for 2.5 h. The mixture was diluted with EtOAc (5 mL), quenched with water (36 mg), 15% NaOH (36 mg) and water (108 mg), the mixture was filtered and the filtrate was concentrated to give 4,4,4-trifluoro-3-phenyl-3-(trifluoromethyl)butan-1-ol (91 mg, 334 μmol, 70.7% yield) as a yellow oil. ¹H NMR (400 MHz, CDCl₃-d) δ 7.59 (br d, 2H), 7.51-7.40 (m, 3H), 3.77-3.70 (m, 2H), 2.70 (t, 2H).

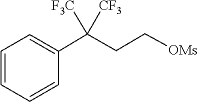 [SMILES: CS(=O)(=O)OCCC(c1ccccc1)(C(F)(F)F)C(F)(F)F]

To a solution of 4,4,4-trifluoro-3-phenyl-3-(trifluoromethyl)butan-1-ol in DCM (3 mL) was added TEA (101 mg, 1.00 mmol, 139 μL, 3 eq) and methylsulfonyl methanesulfonate (87.4 mg, 501 μmol, 1.5 eq) at 0° C. The mixture was stirred at 25° C. for 2 h. The reaction mixture was poured into water (5 mL), extracted with DCM (5 mL×2). The combined organic layers were washed with brine (5 mL), dried over Na₂SO₄, filtered and concentrated under reduced pressure to give 4,4,4-trifluoro-3-phenyl-3-(trifluoromethyl)butyl methanesulfonate (95 mg, 271 μmol, 81.1% yield) as a yellow oil. ¹H NMR (400 MHz, CDCl₃-d) δ 7.58 (br d, 2H), 7.53-7.42 (m, 3H), 4.31-4.22 (m, 2H), 2.99 (s, 3H), 2.90 (t, 2H).

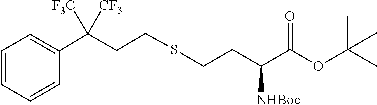 [SMILES: CC(C)(C)OC(=O)N[C@@H](CCSCCC(c1ccccc1)(C(F)(F)F)C(F)(F)F)C(=O)OC(C)(C)C]

A mixture of 4,4,4-trifluoro-3-phenyl-3-(trifluoromethyl)butyl methanesulfonate (95 mg, 271 μmol, 1 eq), tert-butyl (tert-butoxycarbonyl)-L-homocysteinate (63.2 mg, 217 μmol, 0.8 eq), K₂CO₃ (56.2 mg, 407 μmol, 1.5 eq) and KI (45.0 mg, 271 μmol, 1 eq) in DMF (2 mL) was degassed and purged with Ar for 3 times in glove box, and then the mixture was stirred at 70° C. for 12 h under Ar atmosphere. The reaction mixture was poured into water (15 mL), extracted with EtOAc (15 mL×2). The combined organic layers were washed with brine (20 mL), dried over Na₂SO₄, filtered, and concentrated under reduced pressure. The residue was by prep-TLC (SiO₂, petroleum ether/EtOAc=5:1) to give tert-butyl N-(tert-butoxycarbonyl)-S-(4,4,4-trifluoro-3-phenyl-3-(trifluoromethyl)butyl)-L-homocysteinate (125 mg, 229 μmol, 84.5% yield) as a yellow oil.

¹H NMR (400 MHz, CDCl₃-d) δ 7.55-7.51 (m, 2H), 7.50-7.43 (m, 3H), 5.10 (br d, 1H), 4.29 (br d, 1H), 2.68-2.57 (m, 4H), 2.51-2.43 (m, 2H), 2.13-2.00 (m, 1H), 1.91-1.78 (m, 1H), 1.46 (s, 9H), 1.45 (s, 9H).

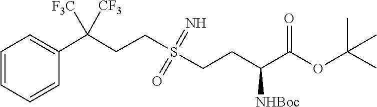 [SMILES: CC(C)(C)OC(=O)N[C@@H](CCS(=N)(=O)CCC(c1ccccc1)(C(F)(F)F)C(F)(F)F)C(=O)OC(C)(C)C]

A mixture of tert-butyl N-(tert-butoxycarbonyl)-S-(4,4,4-trifluoro-3-phenyl-3-(trifluoromethyl)butyl)-L-homocysteinate (125 mg, 229 μmol, 1 eq), ammonium carbamate (143 mg, 1.83 mmol, 8 eq) and PhI(OAc)₂ (295 mg, 916 μmol, 4 eq) in i-PrOH (3 mL) was stirred at 25° C. for 12 h. The reaction mixture was concentrated under reduced pressure to give a residue. The residue was diluted with water (10 mL), extracted with EtOAc (10 mL×2). The combined organic layers were washed with brine (10 mL), dried over Na₂SO₄, filtered, and concentrated under reduced pressure. The residue was purified by prep-TLC (SiO₂, petroleum ether/EtOAc=2:1) to give tert-butyl (2S)-2-((tert-butoxycarbonyl)amino)-4-(4,4,4-trifluoro-3-phenyl-3-(trifluoromethyl)butylsulfonimidoyl)butanoate (110 mg, 191 μmol, 83.2% yield) as a yellow oil. ¹H NMR (400 MHz, CDCl₃-d) δ 7.62-7.56 (m, 2H), 7.54-7.44 (m, 3H), 5.31-5.24 (m, 1H), 4.37-4.21 (m, 1H), 3.43-3.17 (m, 3H), 3.14-2.91 (m, 3H), 2.46-2.36 (m, 1H), 2.17-2.09 (m, 1H), 1.48 (s, 9H), 1.44 (d, 9H).

tert-butyl (2S)-2-((tert-butoxycarbonyl)amino)-4-(4,4,4-trifluoro-3-phenyl-3-(trifluoromethyl)butylsulfonimidoyl)butanoate (110 mg, 191 μmol, 1 eq) in HCl/dioxane (10 mL) (6 M) was stirred at 25° C. for 12 h. The reaction mixture was concentrated under reduced pressure. The residue was purified by prep-HPLC (column: Phenomenex C18 (75×30 mm, 3 μm); mobile phase: [water (NH₄HCO₃)-MeCN]; gradient: 20-50% B, 10 min) to give (2S)-2-amino-4-(4,4,4-trifluoro-3-phenyl-3-(trifluoromethyl)butylsulfonimidoyl)butanoic acid (29.7 mg, 69.8 μmol, 36.6% yield) as a white solid. LCMS: Rt=1.916 min, (ES⁺) m/z (M+H)⁺=421.1, HPLC conditions: C. ¹H NMR (400 MHz, MeOD-d₄) δ 7.65 (br d, 2H), 7.57-7.48 (m, 3H), 3.69 (td, 1H), 3.53-3.33 (m, 2H), 3.20-3.07 (m, 2H), 3.06-2.90 (m, 2H), 2.40-2.22 (m, 2H).

### Exemplary Embodiment 1aa55

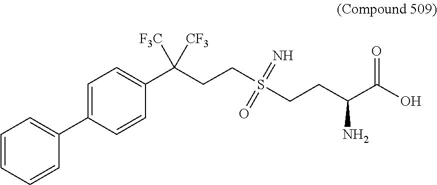 [SMILES: N=S(=O)(CC[C@H](N)C(=O)O)CCC(c1ccc(-c2ccccc2)cc1)(C(F)(F)F)C(F)(F)F]

### (2S)-4-(3-([1,1′-biphenyl]-4-yl)-4,4,4-trifluoro-3-(trifluoromethyl)butylsulfonimidoyl)-2-aminobutanoic acid

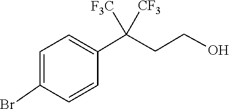 [SMILES: OCCC(c1ccc(Br)cc1)(C(F)(F)F)C(F)(F)F]

To a solution of methyl 3-(4-bromophenyl)-4,4,4-trifluoro-3-(trifluoromethyl)butanoate (1.03 g, 2.71 mmol, 1 eq) in THE (10 mL) was added LAH (123 mg, 3.25 mmol, 1.2 eq) at −40° C. The mixture was stirred at −40° C. for 1 h. The mixture was diluted with EtOAc (10 mL), quenched with water (124 mg), 15% aqueous NaOH (124 mg) and water (372 mg), the mixture was filtered and the filtrate was concentrated to give 3-(4-bromophenyl)-4,4,4-trifluoro-3-(trifluoromethyl)butan-1-ol (964 mg, crude) as a yellow oil. ¹H NMR (400 MHz, CDCl₃-d) δ 7.66-7.56 (m, 2H), 7.46 (br d, 2H), 3.70 (t, 2H), 2.72-2.60 (m, 2H).

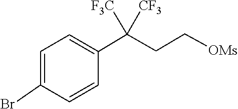 [SMILES: CS(=O)(=O)OCCC(c1ccc(Br)cc1)(C(F)(F)F)C(F)(F)F]

To a solution of 3-(4-bromophenyl)-4,4,4-trifluoro-3-(trifluoromethyl)butan-1-ol (964 mg, 2.75 mmol, 1 eq) in DCM (10 mL) was added TEA (833 mg, 8.24 mmol, 1.15 mL, 3 eq) and methylsulfonyl methanesulfonate (717 mg, 4.12 mmol, 1.5 eq) at 0° C. The mixture was stirred at 25° C. for 2 h. The reaction mixture was poured into water (20 mL), extracted with DCM (20 mL×2). The combined organic layers were washed with brine (20 mL), dried over Na₂SO₄, filtered and concentrated under reduced pressure to give 3-(4-bromophenyl)-4,4,4-trifluoro-3-(trifluoromethyl)butyl methanesulfonate (1.15 g, 2.68 mmol, 97.6% yield) as a yellow oil. ¹H NMR (400 MHz, CDCl₃-d) δ 7.67-7.57 (m, 2H), 7.46 (br d, 2H), 4.30-4.18 (m, 2H), 2.99 (br d, 3H), 2.90-2.78 (m, 2H).

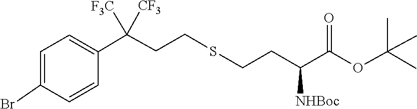 [SMILES: CC(C)(C)OC(=O)N[C@@H](CCSCCC(c1ccc(Br)cc1)(C(F)(F)F)C(F)(F)F)C(=O)OC(C)(C)C]

A mixture of 3-(4-bromophenyl)-4,4,4-trifluoro-3-(trifluoromethyl)butyl methanesulfonate (1.15 g, 2.68 mmol, 1 eq), tert-butyl (tert-butoxycarbonyl)-L-homocysteinate (1.17 g, 4.02 mmol, 1.5 eq), K₂CO₃ (1.11 g, 8.04 mmol, 3 eq) and KI (890 mg, 5.36 mmol, 2 eq) in DMF (10 mL) was degassed and purged with Ar for 3 times, and then the mixture was stirred at 70° C. 12 h under Ar atmosphere. The reaction mixture was poured into water (50 mL), extracted with DCM (50 mL×2). The combined organic layers were washed with brine (50 mL), dried over Na₂SO₄, filtered, and concentrated under reduced pressure. The residue was purified by column chromatography (SiO₂, petroleum ether/EtOAc=98:2 to 80:20) to give tert-butyl S-(3-(4-bromophenyl)-4,4,4-trifluoro-3-(trifluoromethyl)butyl)-N-(tert-butoxycarbonyl)-L-homocysteinate (1 g, 1.60 mmol, 59.7% yield) as a yellow oil. ¹H NMR (400 MHz, CDCl₃-d) δ 7.64-7.58 (m, 2H), 7.40 (d, 2H), 5.11 (br d, 1H), 4.30 (br d, 1H), 2.66-2.57 (m, 4H), 2.49-2.42 (m, 2H), 2.12-2.00 (m, 1H), 1.91-1.78 (m, 1H), 1.46 (s, 9H), 1.45 (s, 9H).

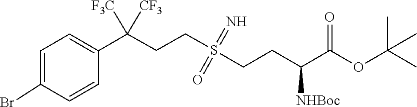 [SMILES: CC(C)(C)OC(=O)N[C@@H](CCS(=N)(=O)CCC(c1ccc(Br)cc1)(C(F)(F)F)C(F)(F)F)C(=O)OC(C)(C)C]

A mixture of tert-butyl S-(3-(4-bromophenyl)-4,4,4-trifluoro-3-(trifluoromethyl)butyl)-N-(tert-butoxycarbonyl)-L-homocysteinate (500 mg, 801 μmol, 1 eq), ammonium carbamate (500 mg, 6.41 mmol, 8 eq) and PhI(OAc)₂ (1.03 g, 3.20 mmol, 4 eq) in i-PrOH (5 mL) was stirred at 25° C. for 12 h. The reaction mixture was concentrated under reduced pressure. The reaction mixture was diluted with water (20 mL) and extracted with EtOAc (20 mL×2). The combined organic layers were washed with brine (20 mL), dried over Na₂SO₄, filtered, and concentrated under reduced pressure. The residue was purified by column chromatography (SiO₂, petroleum ether/EtOAc=94:6 to 70:30) to give tert-butyl (2S)-4-(3-(4-bromophenyl)-4,4,4-trifluoro-3-(trifluoromethyl)butylsulfonimidoyl)-2-((tert-butoxycarbonyl)amino)butanoate (350 mg, 534 μmol, 66.7% yield) as a yellow oil. ¹H NMR (400 MHz, CDCl₃-d) δ 7.64 (br d, 2H), 7.48 (br d, 2H), 5.29-5.19 (m, 1H), 4.36-4.22 (m, 1H), 3.33-3.10 (m, 3H), 3.07-2.88 (m, 3H), 2.47-2.34 (m, 1H), 2.15-2.07 (m, 1H), 1.48 (s, 9H), 1.44 (br d, 9H).

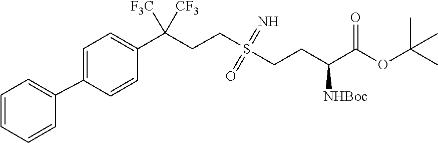 [SMILES: CC(C)(C)OC(=O)N[C@@H](CCS(=N)(=O)CCC(c1ccc(-c2ccccc2)cc1)(C(F)(F)F)C(F)(F)F)C(=O)OC(C)(C)C]

A mixture of tert-butyl (2S)-4-(3-(4-bromophenyl)-4,4,4-trifluoro-3-(trifluoromethyl)butylsulfonimidoyl)-2-((tert-butoxycarbonyl)amino)butanoate (100 mg, 153 μmol, 1 eq), phenylboronic acid (37.2 mg, 305 μmol, 2 eq), Na₂CO₃ (48.5 mg, 458 μmol, 3 eq) and Pd(dppf)Cl₂ (12.5 mg, 15.3 μmol, 0.1 eq) in t-BuOH (2.1 mL) and H₂O (0.7 mL) was degassed and purged with N₂ for 3 times, and then the mixture was stirred at 90° C. for 2 h under N₂ atmosphere. The reaction mixture was poured into water (10 mL) and extracted with EtOAc (10 mL×2). The combined organic layers were washed with brine (10 mL), dried over Na₂SO₄, filtered, and concentrated under reduced pressure. The residue was purified by prep-TLC (SiO₂, petroleum ether/EtOAc=3:1) to give tert-butyl (2S)-4-(3-([1,1′-biphenyl]-4-yl)-4,4,4-trifluoro-3-(trifluoromethyl)butylsulfonimidoyl)-2-((tert-butoxycarbonyl)amino)butanoate (80 mg, 123 μmol, 80.3% yield) as a yellow oil. ¹H NMR (400 MHz, CDCl₃-d) δ 7.74-7.60 (m, 6H), 7.48 (t, 2H), 7.42-7.38 (m, 1H), 5.30-5.19 (m, 1H), 4.36-4.25 (m, 1H), 3.27-2.88 (m, 6H), 2.40 (br dd, 1H), 2.13 (br dd, 1H), 1.47 (s, 9H), 1.43 (s, 9H).

tert-butyl (2S)-4-(3-([1,1′-biphenyl]-4-yl)-4,4,4-trifluoro-3-(trifluoromethyl)butylsulfonimidoyl)-2-((tert-butoxycarbonyl)amino)butanoate (80 mg, 123 μmol, 1 eq) in HCl/dioxane (10 mL, 6 M) was stirred at 25° C. for 4 h. The reaction mixture was concentrated under reduced pressure. The residue was purified by prep-HPLC (column: Waters Xbridge BEH C18 (100×30 mm, 10 μm); mobile phase: [water (NH₄HCO₃)-MeCN]; gradient: 20-50% B, 8 min) to give (2S)-4-(3-([1,1′-biphenyl]-4-yl)-4,4,4-trifluoro-3-(trifluoromethyl)butylsulfonimidoyl)-2-aminobutanoic acid (27.0 mg, 50.4 μmol, 41.1% yield) as a white solid. LCMS: Rt=2.235 min, (ES⁺) m/z (M+H)⁺=497.2, HPLC conditions: C. ¹H NMR (400 MHz, MeOD-d₄) δ 7.84-7.78 (m, 2H), 7.75-7.65 (m, 4H), 7.52-7.44 (m, 2H), 7.43-7.35 (m, 1H), 3.72 (td, 1H), 3.51-3.35 (m, 2H), 3.25-3.12 (m, 2H), 3.10-2.93 (m, 2H), 2.42-2.25 (m, 2H).

### Exemplary Embodiment 1aa56

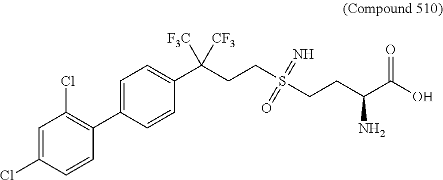 [SMILES: N=S(=O)(CC[C@H](N)C(=O)O)CCC(c1ccc(-c2ccc(Cl)cc2Cl)cc1)(C(F)(F)F)C(F)(F)F]

### (2S)-2-amino-4-(3-(2′,4′-dichloro-[1,1′-biphenyl]-4-yl)-4,4,4-trifluoro-3-(trifluoromethyl)butylsulfonimidoyl)butanoic acid

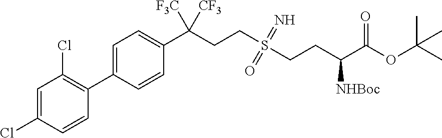 [SMILES: CC(C)(C)OC(=O)N[C@@H](CCS(=N)(=O)CCC(c1ccc(-c2ccc(Cl)cc2Cl)cc1)(C(F)(F)F)C(F)(F)F)C(=O)OC(C)(C)C]

A mixture of tert-butyl (2S)-4-[[3-(4-bromophenyl)-4,4,4-trifluoro-3-(trifluoromethyl)butyl]sulfonimidoyl]-2-(tert-butoxycarbonylamino)butanoate (100 mg, 153 μmol, 1 eq), (2,4-dichlorophenyl)boronic acid (58.2 mg, 305 μmol, 2 q), Na₂CO₃ (48.5 mg, 458 μmol, 3 eq) and Pd(dppf)Cl₂ (12.6 mg, 15.3 μmol, 0.1 eq) in t-BuOH (2.1 mL) and H₂O (0.7 mL) was degassed and purged with N₂ for 3 times, and then the mixture was stirred at 90° C. for 2 h under N₂ atmosphere. The mixture was added EtOAc (30 mL), dried over Na₂SO₄, filtered and concentrated. The residue was purified by prep-TLC (SiO₂, DCM/MeOH=10:1) to give tert-butyl (2S)-2-((tert-butoxycarbonyl)amino)-4-(3-(2′,4′-dichloro-[1,1′-biphenyl]-4-yl)-4,4,4-trifluoro-3-(trifluoromethyl)butylsulfonimidoyl)butanoate (102 mg, 141.36 μmol, 92.66% yield) as a white solid. ¹H NMR (400 MHz, CDCl₃-d) δ 7.67 (br d, 2H), 7.58-7.51 (m, 2H), 7.48-7.38 (m, 1H), 7.32 (br d, 2H), 5.43-5.14 (m, 1H), 4.43-4.11 (m, 1H), 3.36 (br s, 2H), 3.25-3.11 (m, 1H), 3.07-2.85 (m, 2H), 2.51-2.35 (m, 1H), 2.21-2.09 (m, 1H), 1.81-1.58 (m, 18H).

A solution of tert-butyl (2S)-2-((tert-butoxycarbonyl)amino)-4-(3-(2′,4′-dichloro-[1,1′-biphenyl]-4-yl)-4,4,4-trifluoro-3-(trifluoromethyl)butylsulfonimidoyl)butanoate (100 mg, 139 μmol, 1 q) in HCl/dioxane (6 M, 20 mL) was stirred at 25° C. for 4 h. The mixture was concentrated. The residue was purified by prep-HPLC: (column: column: Waters Xbridge Prep OBD C18 (150×40 mm, 10 μm); mobile phase: [water (NH₄HCO₃)-MeCN]; gradient: 25-65% B) to give (2S)-2-amino-4-(3-(2′,4′-dichloro-[1,1′-biphenyl]-4-yl)-4,4,4-trifluoro-3-(trifluoromethyl)butylsulfonimidoyl)butanoic acid (38 mg, 67.21 μmol, 48.50% yield), as a white solid. LCMS: Rt=2.417 min., (ES⁺) m/z (M+H)⁺=565.1, HPLC conditions: C. ¹H NMR (400 MHz, MeOD-d₄) δ 7.75 (br d, 2H), 7.64-7.60 (m, 3H), 7.46-7.40 (m, 2H), 3.70 (td, 1H), 3.46-3.35 (m, 2H), 3.19 (td, 2H), 3.12-2.94 (m, 2H), 2.40-2.28 (m, 2H).

### Exemplary Embodiment 1aa57

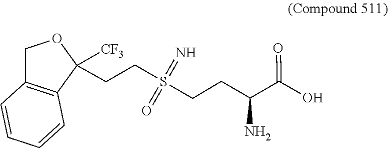 [SMILES: N=S(=O)(CC[C@H](N)C(=O)O)CCC1(C(F)(F)F)OCc2ccccc21]

### (2S)-2-amino-4-(2-(1-(trifluoromethyl)-1,3-dihydroisobenzofuran-1-yl)ethylsulfonimidoyl)butanoic acid

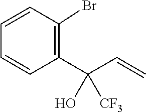

To a solution of 1-(2-bromophenyl)-2,2,2-trifluoroethan-1-one (4.20 g, 16.6 mmol, 1 eq) in THE (60 mL) was added bromo(vinyl)magnesium (1 M, 33.3 mL, 2.01 eq) at −65° C. The mixture was stirred at 0° C. for 2 h. The reaction mixture was quenched by addition of saturated aqueous NH₄Cl (25 mL) at 0° C. and the mixture was extracted with EtOAc (60 mL×2). The combined organic layers were washed with brine (40 mL), dried over Na₂SO₄, filtered, and concentrated under reduced pressure. The residue was purified by column chromatography (SiO₂, petroleum ether/EtOAc=92:8) to give 2-(2-bromophenyl)-1,1,1-trifluorobut-3-en-2-ol (4.2 g, 14.94 mmol) as a colorless oil. ¹H NMR (400 MHz, CDCl₃-d) δ 7.79-7.61 (m, 2H), 7.42-7.32 (m, 1H), 7.23 (dt, 1H), 6.49 (dd, 1H), 5.63-5.45 (m, 2H), 3.57 (s, 1H).

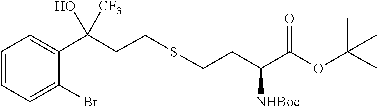 [SMILES: CC(C)(C)OC(=O)N[C@@H](CCSCCC(O)(c1ccccc1Br)C(F)(F)F)C(=O)OC(C)(C)C]

To a solution of 2-(2-bromophenyl)-1,1,1-trifluorobut-3-en-2-ol (700 mg, 2.49 mmol, 1 eq) and tert-butyl (tert-butoxycarbonyl)-L-homocysteinate (870 mg, 2.99 mmol, 1.2 eq) in H₂O (0.3 mL) and MeOH (0.6 mL) was added AIBN (49.1 mg, 298 μmol, 0.12 eq). The mixture was stirred at 60° C. for 12 h under Ar. The reaction mixture was concentrated under reduced pressure. The residue was purified by column chromatography (SiO₂, petroleum ether/EtOAc=95:2) to give tert-butyl S-(3-(2-bromophenyl)-4,4,4-trifluoro-3-hydroxybutyl)-N-(tert-butoxycarbonyl)-L-homocysteinate (800 mg, 1.40 mmol, 56.11% yield) as a yellow oil. ¹H NMR (400 MHz, CDCl₃-d) δ 7.89-7.77 (m, 1H), 7.65 (br d, 1H), 7.40 (br t, 1H), 7.26-7.18 (m, 1H), 5.23-4.99 (m, 1H), 4.68-4.48 (m, 1H), 4.38-4.16 (m, 1H), 3.35-3.05 (m, 1H), 2.77-2.45 (m, 4H), 2.31 (dt, 1H), 2.13-1.78 (m, 2H), 1.53-1.38 (m, 18H).

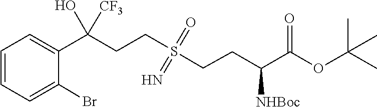 [SMILES: CC(C)(C)OC(=O)N[C@@H](CCS(=N)(=O)CCC(O)(c1ccccc1Br)C(F)(F)F)C(=O)OC(C)(C)C]

To a solution of tert-butyl S-(3-(2-bromophenyl)-4,4,4-trifluoro-3-hydroxybutyl)-N-(tert-butoxycarbonyl)-L-homocysteinate (800 mg, 1.40 mmol, 1 eq) in i-PrOH (10 mL) was added PhI(OAc)₂ (1.80 g, 5.59 mmol, 4 eq) and ammonium carbamate (872 mg, 11.2 mmol, 8 eq). The mixture was stirred at 25° C. for 16 h. The reaction mixture was concentrated under reduced pressure. The mixture was poured into water (10 mL) and extracted with EtOAc (15 mL×3). The combined organic layers were dried over Na₂SO₄, filtered, and concentrated under reduced pressure. The residue was purified by column chromatography (SiO₂, petroleum ether/EtOAc=60:40) to give tert-butyl(2S)-4-(3-(2-bromophenyl)-4,4,4-trifluoro-3-hydroxybutylsulfonimidoyl)-2-((tert-butoxycarbonyl)amino)butanoate (600 mg, 994.22 μmol, 71.15% yield) as a yellow oil. ¹H NMR (400 MHz, CDCl₃-d) δ 8.16-7.97 (m, 1H), 7.65 (dd, 1H), 7.42 (t, 1H), 7.24 (dt, 1H), 5.29-5.07 (m, 1H), 4.42-4.18 (m, 1H), 3.84-3.52 (m, 1H), 3.42-2.88 (m, 5H), 2.85-2.56 (m, 1H), 2.49-2.22 (m, 1H), 2.17-2.06 (m, 1H), 1.50-1.42 (m, 18H).

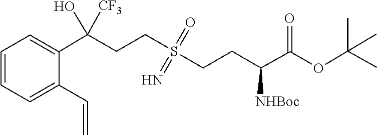 [SMILES: C=Cc1ccccc1C(O)(CCS(=N)(=O)CC[C@H](NC(=O)OC(C)(C)C)C(=O)OC(C)(C)C)C(F)(F)F]

To a solution of tert-butyl(2S)-4-(3-(2-bromophenyl)-4,4,4-trifluoro-3-hydroxybutylsulfonimidoyl)-2-((tert-butoxycarbonyl)amino)butanoate (600 mg, 994 μmol, 1 eq) and potassium vinyltrifluoroborate (532 mg, 3.98 mmol, 4 eq) in H₂O (2 mL) and t-BuOH (6 mL) was added Pd(dppf)Cl₂ (162 mg, 198 μmol, 0.2 eq) and Na₂CO₃ (316 mg, 2.98 mmol, 3 eq). The mixture was stirred at 90° C. for 1 h under N₂. The mixture was poured into water (10 mL) and extracted with EtOAc (10 mL×3). The combined organic layers were dried over Na₂SO₄, filtered, and concentrated under reduced pressure. The residue was purified by prep-TLC (SiO₂, petroleum ether/EtOAc=1:1, R_{f}=0.43) to give tert-butyl (2S)-2-((tert-butoxycarbonyl)amino)-4-(4,4,4-trifluoro-3-hydroxy-3-(2-vinylphenyl)butylsulfonimidoyl)butanoate (380 mg, 690.12 μmol, 69.41% yield) as a yellow oil. ¹H NMR (400 MHz, CDCl₃-d) δ 8.14-8.01 (m, 1H), 7.69-7.57 (m, 1H), 7.43-7.34 (m, 2H), 7.24 (dt, 1H), 5.45 (dd, 1H), 5.36-5.31 (m, 1H), 5.28-5.11 (m, 1H), 4.41-4.16 (m, 1H), 3.31-2.98 (m, 4H), 2.84-2.70 (m, 1H), 2.69-2.54 (m, 1H), 2.47-2.20 (m, 1H), 2.05-1.90 (m, 1H), 1.50-1.44 (m, 18H).

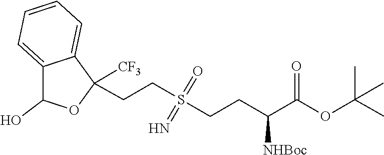 [SMILES: CC(C)(C)OC(=O)N[C@@H](CCS(=N)(=O)CCC1(C(F)(F)F)OC(O)c2ccccc21)C(=O)OC(C)(C)C]

To a solution of tert-butyl (2S)-2-((tert-butoxycarbonyl)amino)-4-(4,4,4-trifluoro-3-hydroxy-3-(2-vinylphenyl)butylsulfonimidoyl)butanoate (300 mg, 544 μmol, 1 eq) in DCM (5 mL) and MeOH (5 mL) was added under 03 (0 psi). The mixture was stirred at −65° C. for 1 h. Then dimethylsulfane (5.08 g, 81.7 mmol, 6.00 mL, 149.9 eq) was added. The mixture was stirred at 25° C. for 1 h. The mixture was poured into water (10 mL) and extracted with EtOAc (15 mL×3). The combined organic layers were dried over Na₂SO₄, filtered, and concentrated under reduced pressure. The residue was purified by prep-TLC (SiO₂, petroleum ether/EtOAc=1:1, R_{f}=0.43) to give tert-butyl (2S)-2-((tert-butoxycarbonyl)amino)-4-(2-(3-hydroxy-1-(trifluoromethyl)-1,3-dihydroisobenzofuran-1-yl)ethylsulfonimidoyl)butanoate (78 mg, 141.15 μmol, 25.91% yield) as a yellow oil. ¹H NMR (400 MHz, CDCl₃-d) δ 7.58-7.43 (m, 3H), 7.42-7.31 (m, 1H), 6.69-6.58 (m, 1H), 5.44-5.16 (m, 1H), 4.33-4.08 (m, 1H), 3.54-2.88 (m, 4H), 2.74-2.58 (m, 2H), 2.46-2.27 (m, 1H), 2.17-2.02 (m, 1H), 1.54-1.40 (m, 18H).

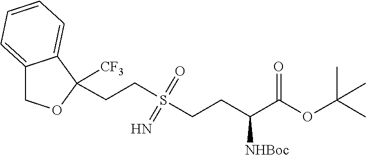 [SMILES: CC(C)(C)OC(=O)N[C@@H](CCS(=N)(=O)CCC1(C(F)(F)F)OCc2ccccc21)C(=O)OC(C)(C)C]

To a solution of tert-butyl (2S)-2-((tert-butoxycarbonyl)amino)-4-(2-(3-hydroxy-1-(trifluoromethyl)-1,3-dihydroisobenzofuran-1-yl)ethylsulfonimidoyl)butanoate (20 mg, 36.2 μmol, 1 eq) in DCM (2 mL) was added Et₃SiH (6.31 mg, 54.3 μmol, 8.67 μL, 1.5 eq) and BF₃·Et₂O (10.3 mg, 72.4 μmol, 8.90 μL, 2 eq) at 0° C. The mixture was stirred at 20° C. for 2 h. The mixture was poured into water (3 mL) and extracted with EtOAc (2 mL×3). The combined organic layers were dried over Na₂SO₄, filtered and concentrated under reduced pressure to give tert-butyl (2S)-2-((tert-butoxycarbonyl)amino)-4-(2-(1-(trifluoromethyl)-1,3-dihydroisobenzofuran-1-yl)ethylsulfonimidoyl)butanoate (50 mg, crude) as a yellow oil.

A mixture of tert-butyl (2S)-2-((tert-butoxycarbonyl)amino)-4-(2-(1-(trifluoromethyl)-1,3-dihydroisobenzofuran-1-yl)ethylsulfonimidoyl)butanoate (50 mg, 114 μmol, 1 eq) in HCl/dioxane (5 M, 10 mL) was stirred at 25° C. for 8 h. The reaction mixture was concentrated under reduced pressure. The residue was purified by prep-HPLC (column: Phenomenex Gemini-NX C18 (75×30 mm, 3 um); mobile phase: [H₂O (10 mM NH₄HCO₃)-MeCN]; gradient: 15-45% B over 9.0 min) to give (2S)-2-amino-4-(2-(1-(trifluoromethyl)-1,3-dihydroisobenzofuran-1-yl)ethylsulfonimidoyl)butanoic acid (5 mg, 11.44 μmol, 9.98% yield) as a white solid. LCMS: Rt=1.987 min, (ES⁺) m/z (M+H)⁺=381.0, HPLC conditions: B. ¹H NMR (400 MHz, MeOD-d₄) δ 7.56-7.36 (m, 4H), 5.28-5.15 (m, 2H), 3.60-3.46 (m, 1H), 3.28-3.04 (m, 3H), 2.66-2.50 (m, 3H), 2.29-2.03 (m, 2H).

### Exemplary Embodiment 1aa58

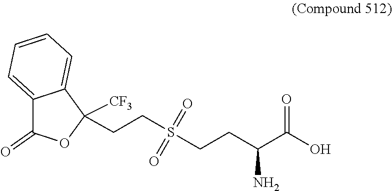 [SMILES: N[C@@H](CCS(=O)(=O)CCC1(C(F)(F)F)OC(=O)c2ccccc21)C(=O)O]

### (2S)-2-amino-4-((2-(3-oxo-1-(trifluoromethyl)-1,3-dihydroisobenzofuran-1-yl)ethyl)sulfonyl)butanoic acid

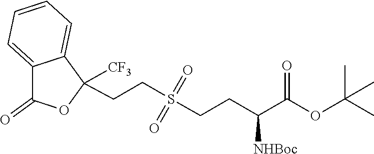 [SMILES: CC(C)(C)OC(=O)N[C@@H](CCS(=O)(=O)CCC1(C(F)(F)F)OC(=O)c2ccccc21)C(=O)OC(C)(C)C]

To a solution of tert-butyl (2S)-2-((tert-butoxycarbonyl)amino)-4-(2-(3-hydroxy-1-(trifluoromethyl)-1,3-dihydroisobenzofuran-1-yl)ethylsulfonimidoyl)butanoate (10 mg, 18.1 μmol, 1 eq) in DCM (2 mL) was added 4 Å MS (20 mg), TPAP (1.91 mg, 5.43 μmol, 0.3 eq) and NMO (8.48 mg, 72.8 μmol, 7.64 μL, 4 eq). The mixture was stirred at 20° C. for 1 hr under N₂. The mixture was filtered and concentrated under reduced pressure. The residue was purified by prep-HPLC (column: Phenomenex Gemini-NX C18 (75×30 mm, 3 μm); mobile phase: [H₂O (10 mM NH₄HCO₃)-MeCN]; gradient: 50-80% B over 9.0 min) to give tert-butyl (2S)-2-((tert-butoxycarbonyl)amino)-4-((2-(3-oxo-1-(trifluoromethyl)-1,3-dihydroisobenzofuran-1-yl)ethyl)sulfonyl)butanoate (10 mg, crude) as a white solid. ¹H NMR (400 MHz, CDCl₃-d) δ 8.01 (d, 1H), 7.89-7.81 (m, 1H), 7.79-7.71 (m, 1H), 7.65 (d, 1H), 5.15 (br s, 1H), 4.22 (br d, 1H), 3.19-2.86 (m, 4H), 2.77-2.63 (m, 1H), 2.58-2.45 (m, 1H), 2.42-2.27 (m, 1H), 2.11-1.93 (m, 1H), 1.46 (d, 18H).

A solution of tert-butyl (2S)-2-((tert-butoxycarbonyl)amino)-4-((2-(3-oxo-1-(trifluoromethyl)-1,3-dihydroisobenzofuran-1-yl)ethyl)sulfonyl)butanoate (10 mg, 18.1 μmol, 1 eq) in HCl/dioxane (4 M, 4.53 μL, 1 eq) was stirred at 30° C. for 16 h. The reaction mixture was concentrated under reduced pressure. The residue was purified by prep-HPLC (column: Phenomenex Gemini-NX C18 (75×30 mm, 3 μm); mobile phase: [H₂O (10 mM NH₄HCO₃)-MeCN]; gradient: 25-55% B over 9.0 min) to give (2S)-2-amino-4-((2-(3-oxo-1-(trifluoromethyl)-1,3-dihydroisobenzofuran-1-yl)ethyl)sulfonyl)butanoic acid (2.67 mg, 6.04 μmol, 33.3% yield) as a white solid. LCMS: Rt=1.822 min, (ES⁺) m/z (M+H)⁺=396.1, HPLC conditions: C. ¹H NMR (400 MHz, MeOD-d₄) δ 8.09-7.74 (m, 4H), 3.77-3.52 (m, 1H), 3.40-3.32 (m, 1H), 3.23 (br d, 1H), 3.17-3.00 (m, 1H), 2.98-2.75 (m, 2H), 2.68 (br d, 1H), 2.31 (br s, 2H).

### Exemplary Embodiment 1aa59 (Peak 1) and 1aa59 (Peak 2)

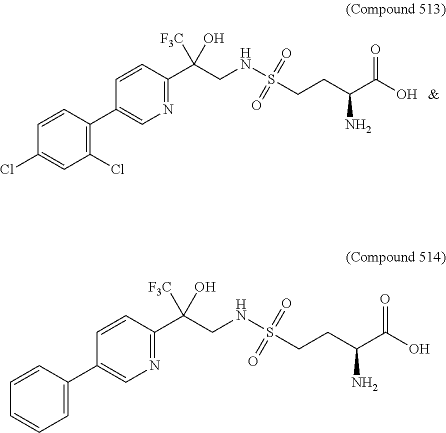

(2S)-2-amino-4-(N-(2-(5-(2,4-dichlorophenyl)pyridin-2-yl)-3,3,3-trifluoro-2-hydroxypropyl)sulfamoyl)butanoic acid & (2S)-2-amino-4-(N-(3,3,3-trifluoro-2-hydroxy-2-(5-phenylpyridin-2-yl)propyl)sulfamoyl)butanoic acid. Compound 513 and Compound 514 are stereoisomers of each other.

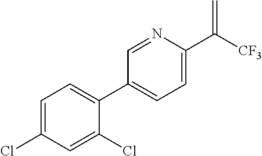 [SMILES: C=C(c1ccc(-c2ccc(Cl)cc2Cl)cn1)C(F)(F)F]

To a solution of 5-bromo-2-(3,3,3-trifluoroprop-1-en-2-yl)pyridine (3 g, 10.5 mmol, 1 eq) and (2,4-dichlorophenyl)boronic acid (3.05 g, 13.7 mmol, 1.3 eq) in dioxane (30 mL) and H₂O (3 mL) was added Pd(dppf)Cl₂ (862 mg, 1.06 mmol, 0.1 eq) and K₂CO₃ (4.38 g, 31.7 mmol, 3 eq). The mixture was stirred at 90° C. for 2 h. The reaction mixture was filtered, and the filtrate was diluted with H₂O 10 mL and extracted with EtOAc (15 mL×3). The combined organic layers were washed with brine (10 mL×3), dried over Na₂SO₄, filtered, and concentrated under reduced pressure to give a residue. The residue was purified by column chromatography (SiO₂, petroleum ether/EtOAc=20:1 to 10:1) to give 5-(2,4-dichlorophenyl)-2-(3,3,3-trifluoroprop-1-en-2-yl)pyridine (1.2 g, 3.77 mmol, 35.74% yield) as a yellow oil.

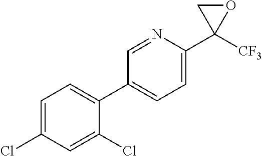 [SMILES: FC(F)(F)C1(c2ccc(-c3ccc(Cl)cc3Cl)cn2)CO1]

To a solution of 5-(2,4-dichlorophenyl)-2-(3,3,3-trifluoroprop-1-en-2-yl)pyridine (1.2 g, 3.77 mmol, 1 eq) in DCM (10 mL) was added m-CPBA (1.07 g, 5.28 mmol, 85% purity, 1.4 eq). The mixture was stirred at 15° C. for 12 h. The reaction mixture was quenched by addition of 5 ml saturated Na₂SO₃ aqueous solution at 0° C. The mixture was then extracted with EtOAc (10 mL×3). The combined organic layers were washed with brine (5 mL×3), dried over Na₂SO₄, filtered, and concentrated under reduced pressure to give a residue. The residue was purified by column chromatography (SiO₂, petroleum ether/EtOAc=20:1 to 19:1) to give 5-(2,4-dichlorophenyl)-2-(2-(trifluoromethyl)oxiran-2-yl)pyridine (270 mg, 808 μmol, 21.4% yield) as a yellow oil. ¹H NMR (400 MHz, CDCl₃-d) δ 8.67 (dd, 1H), 7.83 (dd, 1H), 7.61 (d, 1H), 7.53 (d, 1H), 7.38-7.33 (m, 1H), 7.26-7.23 (m, 1H), 3.48 (d, 1H), 3.25-3.20 (m, 1H).

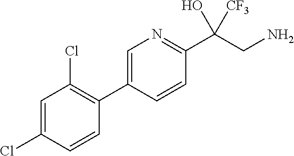 [SMILES: NCC(O)(c1ccc(-c2ccc(Cl)cc2Cl)cn1)C(F)(F)F]

A solution of 5-(2,4-dichlorophenyl)-2-(2-(trifluoromethyl)oxiran-2-yl)pyridine (270 mg, 808 μmol, 1 eq) in NH₃/MeOH (3 mL) was stirred at 15° C. for 12 h. The reaction mixture was concentrated under vacuum. The residue was purified by prep-TLC (SiO₂, EtOAc/MeOH=10:1) to give 3-amino-2-(5-(2,4-dichlorophenyl)pyridin-2-yl)-1,1,1-trifluoropropan-2-ol (240 mg, 683 μmol, 84.5% yield) as a colorless oil. ¹H NMR (400 MHz, CDCl₃-d) δ 8.67 (d, 1H), 7.93 (dd, 1H), 7.69 (d, 1H), 7.56 (d, 1H), 7.42-7.36 (m, 1H), 7.34-7.29 (m, 1H), 3.54-3.46 (m, 1H), 3.41-3.33 (m, 1H).

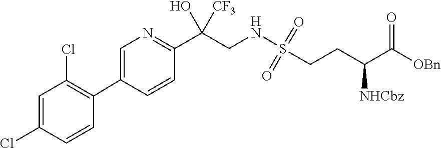 [SMILES: O=C(N[C@@H](CCS(=O)(=O)NCC(O)(c1ccc(-c2ccc(Cl)cc2Cl)cn1)C(F)(F)F)C(=O)OCc1ccccc1)OCc1ccccc1]

To a solution of 3-amino-2-(5-(2,4-dichlorophenyl)pyridin-2-yl)-1,1,1-trifluoropropan-2-ol (150 mg, 427 μmol, 1 eq) in pyridine (5 mL) was added benzyl (S)-2-(((benzyloxy)carbonyl)amino)-4-(chlorosulfonyl)butanoate (545 mg, 1.28 mmol, 3 eq) at 0° C. The mixture was stirred at 0-15° C. for 12 h. The reaction mixture was concentrated to remove solvent. The residue was purified by prep-HPLC (column: Phenomenex Luna C18 (100×40 mm, 3 μm); mobile phase: [H₂O (0.2% FA)-MeCN]; gradient: 65-98% B over 8.0 min) and prep-HPLC (column: Phenomenex Luna C18 (100×30 mm, 3 μm); mobile phase: [H₂O (0.2% FA)-MeCN]; gradient: 35-65% B over 8.0 min) to give benzyl (2S)-2-(((benzyloxy)carbonyl)amino)-4-(N-(2-(5-(2,4-dichlorophenyl)pyridin-2-yl)-3,3,3-trifluoro-2-hydroxypropyl)sulfamoyl)butanoate (88 mg, crude) as a yellow oil.

To a solution of benzyl (2S)-2-(((benzyloxy)carbonyl)amino)-4-(N-(2-(5-(2,4-dichlorophenyl)pyridin-2-yl)-3,3,3-trifluoro-2-hydroxypropyl)sulfamoyl)butanoate (20 mg, 27.0 μmol, 1 eq) in MeOH (1 mL) and 2-methyltetrahydrofuran (1 mL) was added HCl (0.5 M, 270 μL, 5 eq) and Pd/C (20 mg, 18.7 μmol, 10% purity). The mixture was stirred at 30° C. for 4 h under 50 psi. The reaction mixture was filtrated, the filtrate was concentrated under reduced pressure. The residue was purified by prep-HPLC (column: Phenomenex Luna C18 (100×40 mm, 3 μm); mobile phase: [H₂O (0.2% FA)-MeCN]; gradient: 65-98% B over 8.0 min). (2S)-2-amino-4-(N-(2-(5-(2,4-dichlorophenyl)pyridin-2-yl)-3,3,3-trifluoro-2-hydroxypropyl)sulfamoyl)butanoic acid (Peak 1, 2.43 mg, 4.34 μmol, 16.0% yield) was obtained as a white solid. LCMS: Rt=2.268 min, (ES⁺) m/z (M+H)⁺=516.1, HPLC condition: C. ¹H NMR (400 MHz, MeOD-d₄) δ 8.71 (s, 1H), 8.02 (dd, 1H), 7.88 (dd, 1H), 7.65 (s, 1H), 7.49 (s, 2H), 4.05 (dd, 1H), 3.82 (d, 1H), 3.72-3.64 (m, 1H), 3.29-3.07 (m, 2H), 2.28-2.11 (m, 2H). (2S)-2-amino-4-(N-(3,3,3-trifluoro-2-hydroxy-2-(5-phenylpyridin-2-yl)propyl)sulfamoyl)butanoic acid (Peak 2, 16 mg, 33.9 μmol, 36.9% yield) was obtained as a white solid. LCMS: Rt=2.062 min, (ES⁺) m/z (M+H)⁺=448.1. HPLC condition: C. ¹H NMR (400 MHz, MeOD-d₄) δ 8.90 (t, 1H), 8.16 (dd, 1H), 7.84 (dd, 1H), 7.71 (d, 2H), 7.55-7.48 (m, 2H), 7.46-7.40 (m, 1H), 4.03 (d, 1H), 3.82 (d, 1H), 3.73-3.64 (m, 1H), 3.29-3.08 (m, 2H), 2.31-2.06 (m, 2H).

### Exemplary Embodiment 1aa60

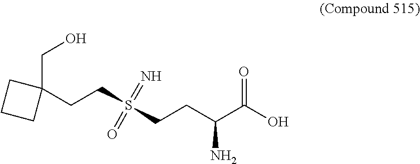 [SMILES: N=[S@@](=O)(CC[C@H](N)C(=O)O)CCC1(CO)CCC1]

### (S)-2-amino-4-((S)-2-(1-(hydroxymethyl)cyclobutyl)ethylsulfonimidoyl)butanoic acid

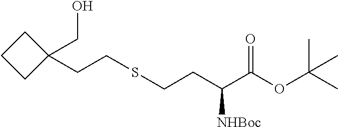 [SMILES: CC(C)(C)OC(=O)N[C@@H](CCSCCC1(CO)CCC1)C(=O)OC(C)(C)C]

To a solution of (S)-1-(2-((4-(tert-butoxy)-3-((tert-butoxycarbonyl)amino)-4-oxobutyl)thio)ethyl)cyclobutane-1-carboxylic acid (3 g, 7.18 mmol, 1 eq) in THE (30 mL) was added BH₃·THF (1 M, 14.37 mL, 2 eq) at 0° C., the mixture was stirred at 15° C. for 2 h under N₂. The mixture was quenched with H₂O (50 mL) and extracted with EtOAc (50 mL×2). The combined organic phase was washed with brine (100 mL), dried with anhydrous Na₂SO₄, filtered, and concentrated. The residue was purified by silica gel chromatography (petroleum ether/EtOAc=10:1, 5:1) to give tert-butyl N-(tert-butoxycarbonyl)-S-(2-(1-(hydroxymethyl)cyclobutyl)ethyl)-L-homocysteinate (3.5 g, crude) as a colorless oil. ¹H NMR (400 MHz, CDCl₃-d) δ 5.15 (br d, 1H), 4.34-4.22 (m, 1H), 3.58 (s, 2H), 2.61-2.53 (m, 2H), 2.51-2.43 (m, 2H), 2.16-2.02 (m, 1H), 1.73 (br s, 9H), 1.46 (d, 18H).

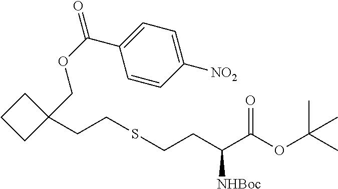 [SMILES: CC(C)(C)OC(=O)N[C@@H](CCSCCC1(COC(=O)c2ccc([N+](=O)[O-])cc2)CCC1)C(=O)OC(C)(C)C]

To a solution of tert-butyl N-(tert-butoxycarbonyl)-S-(2-(1-(hydroxymethyl)cyclobutyl)ethyl)-L-homocysteinate (3.5 g, 8.67 mmol, 1 eq) in DCM (50 mL) was added DIPEA (3.36 g, 26.0 mmol, 4.53 mL, 3 eq), 4-nitrobenzoyl chloride (2.41 g, 13.0 mmol, 1.5 eq) and DMAP (1.06 g, 8.67 mmol, 1 eq) at 0° C., the mixture was stirred at 15° C. for 0.5 h. The mixture was quenched with H₂O (100 mL) and extracted with DCM (100 mL×2). The combined organic phase was washed with brine (100 mL), dried with anhydrous Na₂SO₄, filtered, and concentrated in vacuum. The residue was purified by silica gel chromatography (petroleum ether/EtOAc=100:1, 10:1) to give (S)-(1-(2-((4-(tert-butoxy)-3-((tert-butoxycarbonyl)amino)-4-oxobutyl)thio)ethyl)cyclobutyl)methyl 4-nitrobenzoate (4.3 g, 6.61 mmol, 76.3% yield) as a light yellow oil. ¹H NMR (400 MHz, CDCl₃-d) δ 8.32 (d, 2H), 8.24-8.19 (m, 2H), 5.09 (br d, 1H), 4.36 (s, 2H), 4.27 (br d, 1H), 2.61-2.49 (m, 4H), 2.15-2.03 (m, 1H), 2.02-1.86 (m, 9H), 1.46 (d, 18H).

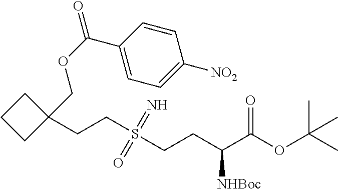 [SMILES: CC(C)(C)OC(=O)N[C@@H](CCS(=N)(=O)CCC1(COC(=O)c2ccc([N+](=O)[O-])cc2)CCC1)C(=O)OC(C)(C)C]

To a mixture of (S)-(1-(2-((4-(tert-butoxy)-3-((tert-butoxycarbonyl)amino)-4-oxobutyl)thio)ethyl)cyclobutyl)methyl 4-nitrobenzoate (3.3 g, 5.97 mmol, 1 eq) in i-PrOH (100 mL) was added PhI(OAc)₂ (4.81 g, 14.9 mmol, 2.5 eq) and ammonia; carbamic acid (2.33 g, 29.9 mmol, 5 eq) at 20° C., the mixture was stirred at 20° C. for 3 h. The mixture was added to H₂O (300 mL) and extracted with EtOAc (100 mL×3). The combined organic phase was washed with brine (200 mL), dried with anhydrous Na₂SO₄, filtered, and concentrated in vacuum. The residue was purified by silica gel chromatography (petroleum ether/EtOAc=100:1 to 1:1) to give (1-(2-((3S)-4-(tert-butoxy)-3-((tert-butoxycarbonyl)amino)-4-oxobutylsulfonimidoyl)ethyl)cyclobutyl)methyl 4-nitrobenzoate (3.89 g, crude) as a light yellow oil. ¹H NMR (400 MHz, CDCl₃-d) δ 8.36-8.29 (m, 2H), 8.24-8.19 (m, 2H), 5.22 (br s, 1H), 4.36 (s, 2H), 4.27 (br d, 1H), 3.24-3.00 (m, 4H), 2.47-2.33 (m, 1H), 2.19-2.08 (m, 3H), 2.04-1.88 (m, 6H), 1.53-1.40 (m, 18H).

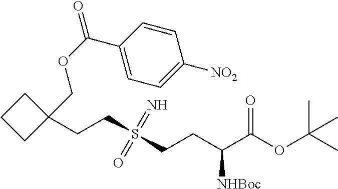 [SMILES: CC(C)(C)OC(=O)N[C@@H](CC[S@@](=N)(=O)CCC1(COC(=O)c2ccc([N+](=O)[O-])cc2)CCC1)C(=O)OC(C)(C)C]

(1-(2-((3S)-4-(tert-butoxy)-3-((tert-butoxycarbonyl)amino)-4-oxobutylsulfonimidoyl)ethyl)cyclobutyl)methyl 4-nitrobenzoate (3.89 g, 6.66 mmol) was purified by prep-HPLC (column: Phenomenex-Cellulose-2 (250×50 mm, 10 μm); mobile phase: [0.1% NH₃·H₂O/MeOH]; gradient: 40% B, 4 min) to give, in order of elution, (1-(2-((R,3S)-4-(tert-butoxy)-3-((tert-butoxycarbonyl)amino)-4-oxobutylsulfonimidoyl)ethyl)cyclobutyl)methyl 4-nitrobenzoate (1.5 g, 2.57 mmol, 38.6% yield) as a white solid and (1-(2-((S,3S)-4-(tert-butoxy)-3-((tert-butoxycarbonyl)amino)-4-oxobutylsulfonimidoyl)ethyl)cyclobutyl)methyl 4-nitrobenzoate (1.6 g, 2.74 mmol, 41.1% yield) as a white solid.

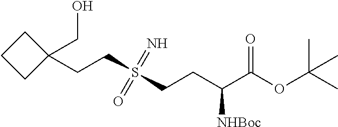 [SMILES: CC(C)(C)OC(=O)N[C@@H](CC[S@@](=N)(=O)CCC1(CO)CCC1)C(=O)OC(C)(C)C]

A mixture of (1-(2-((S,3S)-4-(tert-butoxy)-3-((tert-butoxycarbonyl)amino)-4-oxobutylsulfonimidoyl)ethyl)cyclobutyl)methyl 4-nitrobenzoate (1.5 g, 2.57 mmol, 1 eq), LiOH·H₂O (162 mg, 3.85 mmol, 1.5 eq), in THE (10 mL) and H₂O (2 mL) was degassed and purged 3 times with N₂, and then the mixture was stirred at 20° C. for 2 h under N₂ atmosphere. The organic solvent was removed under reduced pressure. The pH of the aqueous phase was adjusted to 4 by 1 N HCl. The solid was filtered and the aqueous phase was extracted with 10:1 DCM/i-PrOH (10 mL×3). The combined organic layers were dried over Na₂SO₄, filtered, and concentrated under reduced pressure to give tert-butyl (S)-2-((tert-butoxycarbonyl)amino)-4-((S)-2-(1-(hydroxymethyl)cyclobutyl)ethylsulfonimidoyl)butanoate (1.1 g, crude), as a white solid. ¹H NMR (400 MHz, CDCl₃-d) δ 4.17 (br d, 1H), 3.51-3.46 (m, 2H), 3.15-3.01 (m, 2H), 2.99-2.94 (m, 2H), 2.00-1.66 (m, 10H), 1.46-1.35 (m, 18H).

A mixture of give tert-butyl (S)-2-((tert-butoxycarbonyl)amino)-4-((S)-2-(1-(hydroxymethyl)cyclobutyl)ethylsulfonimidoyl)butanoate (1.1 g, 2.53 mmol, 1 eq) in HCl/dioxane (40 mL) was stirred at 20° C. for 4 h. The reaction mixture was concentrated under reduced pressure to give a residue. The residue was purified by prep-HPLC (column: Phenomenex C18 (75×30 mm, 3 μm); mobile phase: [water (FA)-MeCN]; gradient: 1-10% B, 10 min) to give (S)-2-amino-4-((S)-2-(1-(hydroxymethyl)cyclobutyl)ethylsulfonimidoyl)butanoic acid (232 mg, 737 μmol, 45.6% yield, HCl) as a white solid. LCMS: Rt=0.865 min., (ES⁺) m/z (M+H)⁺=279.1, HPLC Conditions: E. ¹H NMR (400 MHz, D₂O) δ 3.83 (s, 1H), 3.53 (s, 2H), 3.49-3.28 (m, 2H), 3.26-3.16 (m, 2H), 2.37-2.27 (m, 2H), 1.95 (s, 2H), 1.90-1.72 (m, 6H).

### Exemplary Embodiment 1aa61

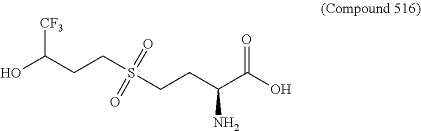 [SMILES: N[C@@H](CCS(=O)(=O)CCC(O)C(F)(F)F)C(=O)O]

### (2S)-2-amino-4-((4,4,4-trifluoro-3-hydroxybutyl)sulfonyl)butanoic acid

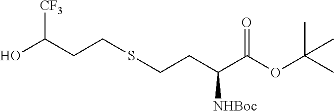 [SMILES: CC(C)(C)OC(=O)N[C@@H](CCSCCC(O)C(F)(F)F)C(=O)OC(C)(C)C]

A mixture of (4,4,4-trifluoro-3-hydroxy-butyl) 4-methylbenzenesulfonate (307 mg, 1.03 mmol, 1 eq), tert-butyl (2S)-2-(tert-butoxycarbonylamino)-4-sulfanyl-butanoate (300 mg, 1.03 mmol, 1 eq), K₂CO₃ (427 mg, 3.09 mmol, 3 eq) and KI (342 mg, 2.06 mmol, 2 eq) in DMF (5 mL) was degassed and purged 3 times with Ar, and then the mixture was stirred at 40° C. for 16 h under Ar atmosphere. The reaction mixture was poured into water (25 mL) and extracted with EtOAc (10 mL×2). The combined organic layers were washed with brine (20 mL), dried over Na₂SO₄, filtered, and concentrated under reduced pressure to give a residue. The residue was purified by column chromatography (SiO₂, petroleum ether/EtOAc=100:0 to 80:20) to give (2S)-tert-butyl 2-((tert-butoxycarbonyl)amino)-4-((4,4,4-trifluoro-3-hydroxybutyl)thio)butanoate (310 mg, 742.55 μmol, 72.13% yield) as a yellow oil. ¹H NMR (400 MHz, CDCl₃-d) δ 5.28-5.07 (m, 1H), 4.33 (br dd, 1H), 4.25-4.06 (m, 1H), 3.14 (br dd, 1H), 2.92-2.76 (m, 1H), 2.75-2.50 (m, 3H), 2.14-1.80 (m, 4H), 1.48 (d, 9H), 1.45 (s, 9H).

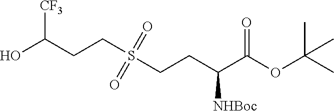 [SMILES: CC(C)(C)OC(=O)N[C@@H](CCS(=O)(=O)CCC(O)C(F)(F)F)C(=O)OC(C)(C)C]

To a solution of (2S)-tert-butyl 2-((tert-butoxycarbonyl)amino)-4-((4,4,4-trifluoro-3-hydroxybutyl)thio)butanoate (155 mg, 371 μmol, 1 eq) in DCM (3 mL) was added m-CPBA (166 mg, 817 μmol, 85% purity, 2.2 eq) at 0° C. under N₂. The mixture was stirred at 20° C. for 3 h. The reaction mixture was poured into water (25 mL) and extracted with EtOAc (10 mL×2). The combined organic layers were washed with brine (20 mL), dried over Na₂SO₄, filtered, and concentrated under reduced pressure to give a residue. The residue was purified by prep-TLC (SiO₂, petroleum ether/EtOAc=1:1) to give (2S)-tert-butyl 2-((tert-butoxycarbonyl)amino)-4-((4,4,4-trifluoro-3-hydroxybutyl)sulfonyl)butanoate (107 mg, 238.05 μmol, 64.12% yield) as a white solid. ¹H NMR (400 MHz, CDCl₃-d) δ 4.37-4.16 (m, 2H), 3.24 (br t, 2H), 3.13-2.97 (m, 2H), 2.53-2.26 (m, 2H), 2.22-2.07 (m, 2H), 1.50 (s, 9H), 1.46 (s, 9H).

A mixture of (2S)-tert-butyl 2-((tert-butoxycarbonyl)amino)-4-((4,4,4-trifluoro-3-hydroxybutyl)sulfonyl)butanoate (107 mg, 238.05 μmol, 1 eq) in HCl/dioxane (20 mL) was degassed and then the mixture was stirred at 20° C. for 12 h. The reaction mixture was concentrated under reduced pressure. The residue was purified by prep-IPLC (column: Phenomenex C18 75*30 mm*3 um; mobile phase: [water (NH₄HCO₃)-MeCN]; B %: 1%-15%, 10 min) to give (2S)-2-amino-4-((4,4,4-trifluoro-3-hydroxybutyl)sulfonyl)butanoic acid (31.7 mg, 107.55 μmol, 45.18% yield, 99.5% purity) as a white solid. LCMS: Rt=0.419 min, (ES⁺) m/z (M+H)⁺=294.1, HPLC Conditions: C. ¹H NMR (400 MHz, D₂O) δ 4.26 (ddd, 1H), 3.89 (t, 1H), 3.52-3.39 (m, 4H), 2.43-2.35 (m, 2H), 2.33-2.24 (m, 1H), 2.15-2.04 (m, 1H).

### Example 2: Biological Testing of Compounds

### GCL Enzyme Profiling Assay:

The GCL enzyme assay was run in 50 mM HEPES, pH 7.5, containing 150 mM NaCl, 30 mM MgCl₂, 0.5 mM EDTA, 1 mM DTT, 0.1 mg/mL BSA, and 0.02% or in the present case 0.2% F-127 in a total assay volume of 10 μL or 10 mL. Compound in pure DMSO was stamped in assay plates (Greiner black 384-well) or appropriate containers using a D300e digital dispenser (TECAN) and the final DMSO concentration was normalized to 1%. A 5 mL, or in the present case, 5 μL mixture of human recombinant GCL (final concentration: 5 nM) and ATP (final concentration: 0.6 mM) was added and the solution was incubated at room temperature (22° C.) for 1 h. The GCL enzyme reaction was initiated by addition of a 5 μL mixture of mono g-glutamate or in the present example, monosodium glutamate (final concentration: 1 mM) and a-aminobutyrate or α-aminobutyrate (final concentration: 1.2 mM). The reaction was stopped after 1 h or in the present case, 2 h by addition of 5 mL, or in the present case, 5 μL ADP GLO Reagent (Promega). The resulting solution was incubated for 2 h at room temperature. This was then followed by addition of 5 mL, or in the present case, 5 μL ADP GLO Detection. After incubation at room temperature for 30 min, the plates were read on a plate reader (PheraStar) using a luminescence protocol to quantify the GCL enzyme activity from each reaction solution. The data was analyzed using a four-parameter logistic equation to calculate IC₅₀. The results are summarized in Table 2 or Table 3.

RKN Cell Profiling Assay with and without Ferroptosis Inhibitors:

RKN cells were suspended in assay medium (F-12 medium supplemented with 10% FBS and penicillin/streptomycin) and seeded at 1,000 cells per well (30 μL total volume) in opaque white 384-well plates (Corning). Cells were allowed to adhere for 24 h at 37° C. (95% humidity, 5% CO₂) and then exposed to compounds disclosed herein for 72 h, or in the present case, 96 h. DMSO stock solutions of compounds were added to cells using a D300e Digital Dispenser (Tecan). The final DMSO concentration in each well was normalized to 0.5% (v/v) after compound treatment. Cell viability was measured using CellTiter-Glo (Promega). After incubation for 15 min at room temperature, luminescence was measured using a PHERAstar FSX microplate reader (BMG Labtech). Ferroptosis rescue experiments were performed as described previously using cells treated with ferrostatin-1 (fer-1, 1.5 μM) when seeding assay plates. Dose-response data were analyzed to determine EC₅₀ values with GraphPad Prism 9 software using a four-parameter logistic equation or CDD Vault (Collaborative Drug Discovery) using the Levenberg-Marquardt algorithm. The results are summarized in Table 2 or Table 3.

### Example 3: Cell Lines and Culture Conditions

Human cancer cell lines are cultured in Ham's F12 medium supplemented with 10% (v/v) fetal bovine serum (FBS), penicillin (100 U/mL), and streptomycin (100 μg/mL). human cancer cells are cultured in RPMI medium supplemented with 10% FBS, penicillin (100 U/mL), and streptomycin (100 μg/mL). Cells are grown in a humidified incubator at 37° C. with 5% carbon dioxide and split every 3-4 days using trypsin/EDTA solution.

Method to add exogenous PUFAs or MUFAs: Exogenous fatty acids were dissolved in DMSO and added to cell culture medium 24 h after seeding cells.

### Example 4: Determination of PUFA Status

Lipidomics are performed using either gas chromatography-mass spectrometry (GC-MS) or direct infusion mass spectrometry.

For GC-MS assessment of cellular PUFA status, the membrane lipids are trans-esterified with 500 μL methanolic HCl, 250 μL n-hexane and 500 μL internal standard (0.8 mg Di-C17-phosphatidylcholine in 1 mL methanol with 0.2% Butylhydroxytoluol as antioxidant). After cooling-off, 500 μL n-hexane and 1 mL Aqua Dest. are added. The upper hexane phase is evaporated with nitrogen. The fatty acid methylesters (FAME) are taken up in 60 μL n-hexane. An aliquot of 1 μL is injected on-column on a Varian CP 3800 gas chromatograph (Varian, Darmstadt, Germany) equipped with an Omegawax™ 320 column (0.32 mm internal diameter, 30 m length) (Supelco, Bellefonte, USA). The column temperature was 200 degrees C.

For direct infusion MS analysis, lipids are extracted using a two-step chloroform/methanol procedure. Samples are spiked with internal lipid standard mixture containing: cardiolipin 16:1/15:0/15:0/15:0 (CL), ceramide 18:1; 2/17:0 (Cer), diacylglycerol 17:0/17:0 (DAG), hexosylceramide 18:1; 2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1; 2/12:0; 0 (SM), triacylglycerol 17:0/17:0/17:0 (TAG). After extraction, the organic phase is transferred to an infusion plate and dried in a speed vacuum concentrator. The dried extract is re-suspended in 7.5 mM ammonium acetate in chloroform/methanol/propanol (1:2:4, V:V:V) and the second step dry extract is re-suspended in a 33% ethanol solution of methylamine in chloroform/methanol (0.003:5:1; V:V:V). Samples are analyzed by direct infusion on a QExactive mass spectrometer (ThermoFisher Scientific) equipped with a TriVersa NanoMate ion source (Advion Biosciences). Samples are analyzed in both positive and negative ion modes with a resolution of Rm/z=200=280000 for MS and Rm/z=200=17500 for tandem mass spectrometry (MS-MS) assays, in a single acquisition. MS-MS is triggered by an inclusion list encompassing corresponding MS mass ranges scanned in 1 Da increments.

### Example 5: Cell Line Profiling with a Ferroptosis-Inducing Agent with and without a Rescue Agent

Cell viability assays are performed by seeding 1,000 cells per well (30 μl volume) in opaque white 384-well plates (Corning). Cells are allowed to adhere for 24 h, after which they are exposed to compounds for 72 hours. DMSO stock solutions of compounds are added to cells using a CyBio Well Vario liquid dispenser (Analytik Jena AG). Cellular ATP levels are measured using CellTiter-Glo (Promega) as a surrogate for viability. Rescue assays are performed using rescue agents-ferrostatin-1 (fer-1, 1.5 μM), liproxstatin-1 (lip-1, 1 μM), deferoxamine (DFO, 50 μM), and other ferroptosis inhibitors added to cells at the time of addition to assay plates.

### Knocking Down Targets Using Genetic Reagents+/−Fer-I

For lentiviral shRNA production, 293-T cells are seeded in 6-well dishes in antibiotic free media (280,000 cells/well). The next day, cells were transfected using FuGENE with the appropriate shRNA encoding plasmid (450 ng), viral packaging plasmid (p-Delta8.9, 400 ng), and viral envelope plasmid (p-VSV-G, 45 ng). After 24 h, the medium is removed and replaced with fresh medium. Three collections of viral supernatant per shRNA are made over 36 h and pooled. The combined supernatant is centrifuged, aliquoted, and stored at −80 degrees C. until virus infection.

Lentiviral infections are performed by seeding cells for 12 h and replacing the media with media supplemented with polybrene (8 μg/mL) and an aliquot of the viral supernatant. Plates are incubated for 48 h and the media is replaced with media containing 1.5 μg/mL puromycin and incubated at 37 degrees C. for 48 h. Knockdown is assessed by immunoblotting and RT-qPCR.

### Knocking Out Targets Using Genetic Reagents+/−Fer-I

For generation of cell lines with gene knockouts, lentiviruses are generated by overnight polyethylenimine transfection of Leni-X 293T cells with target lentiviral plasmid and packaging plasmids pCMV-dR8.2 dvpr and pCMV-VSV-G in DMEM supplemented with 10% FBS. The next day, the medium is changed to fresh DMEM with 10% FBS. After 24 and 48 h, the virus-containing medium is collected and filtered with a 0.45 μm polyethersulfone filter, combined, and stored at −80 degrees C. until virus infection.

Cells are transduced with pLenti-CRISPR—V2 encoding the appropriate sgRNAs for the target genes using 2 μg/mL of polybrene followed by puromycin selection (1 μg/mL) for 4 days in the presence of ferrostatin-1 (1 μM). Protein knockout is verified via immunoblotting.

### Example 6: Use of C11-BODIPY to Show Lipid Peroxidation as an Indicator of Ferroptosis

Imaging assay: human cancer cells are seeded at 5,000 cells per well in a CellCarrier Ultra 96-well plate (Perkin-Elmer) in 150 μl of RPMI medium with 10% FBS. Cells are incubated for 24 h at 37° C. and then treated with the indicated compounds or DMSO (90 min, 37° C.). During the last 30 min of incubation, 60 nM DRAQ7 (Abcam), 1 g ml-1 Hoechst 33342 (ThermoFisher) and 1 μM BODIPY 581/591 C11 (ThermoFisher) dyes are added. Cells are imaged using an Opera Phenix High-Content Screening System (Perkin-Elmer) equipped with 405, 488, 560 and 647 nm lasers. Image analysis is conducted with Harmony High-Content Imaging and Analysis software (Perkin-Elmer).

### Use of C11-BODIPY to Show Lipid Peroxidation (Flow Cytometry Assay)

Human cancer cells are seeded at 15,000 cells per well in 96-well plates in RPMI medium with 10% FBS. After 48 h, culture media is replaced with 200 μl media containing either DMSO or the indicated inhibitor (10 M) and 1 M anti-ferroptosis rescue agent (where indicated). Cultures are incubated at 37° C. for 2 h. Thirty minutes before the end of the incubation period, 10 μM BODIPY 581/591 C₁₁ (Molecular Probes no. C10445) is added to cells. Cells are gathered in 200 μl PBS+0.1% BSA and subjected to flow cytometry analysis (BD FACSCanto II).

### Example 7 Microdosing Tumors with Ferroptosis-Inducing Agents

For the allograft study, cancer cell line-derived tumor cells were injected into the flanks of male C57BL6/J mice. Assays were initiated when the tumor diameter was approximately 6-7 mm.

Microdose drug delivery was performed for the assay described herein. The compounds in Table 22 were packed into device reservoirs using a tapered metal needle. Reservoirs were loaded for initial release of liproxstatin-1 (where included) followed by a 4-6 h delayed release of ferroptosis inducers. Devices were prepared for dose administration into mouse tumors. Devices delivered the ferroptosis inducing agent for 24-72 hours in the tissue. The tumor was then excised, and the tissue was snap frozen with liquid nitrogen. Tissue was sectioned using a standard cryotome, and tissue slices of 20 μm in thickness were collected from each reservoir for analysis by immunoassays, transcriptomics, and metabolomic assays.

TABLE 22
Microdosing Ferroptosis-Inducing Agents and Conditions

| | | | | Target |
|---|---|---|---|---|
| Reservoir | Compound | | | concentration |
| # | Name | Loading | Rescue agent | range (μM) |
| 1 | RSL3 | high | | 1-100 |
| 2 | RSL3 | high | liproxstatin-1 | 1-100 |
| | | | (lip-1) | |
| 3 | RSL3 | low | | 1-10 |
| 4 | RSL3 | low | lip-1 | 1-10 |
| 5 | ML-210 | high | | 1-10 |
| 6 | ML-210 | high | lip-1 | 1-10 |
| 7 | JKE-1674 | high | | 1-10 |
| 8 | JKE-1674 | high | lip-1 | 1-10 |
| 9 | erastin | high | | 5-500 |
| 10 | erastin | high | lip-1 | 5-500 |
| 11 | L-BSO | high | | 100-150 |
| 12 | L-BSO | high | lip-1 | 100-150 |
| 13 | jacaric acid | high | | 1-10 |
| 14 | jacaric acid | high | lip-1 | 1-10 |
| 16 | Empty | | lip-1 | 1-10 |

### Example 8: Ferroptosis Induction by Sustained, Targeted Administration of L-BSO

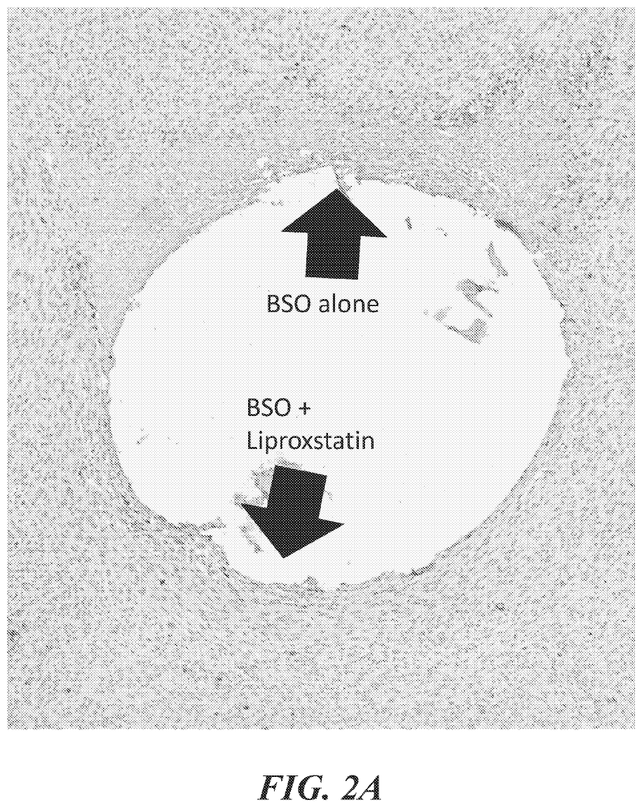

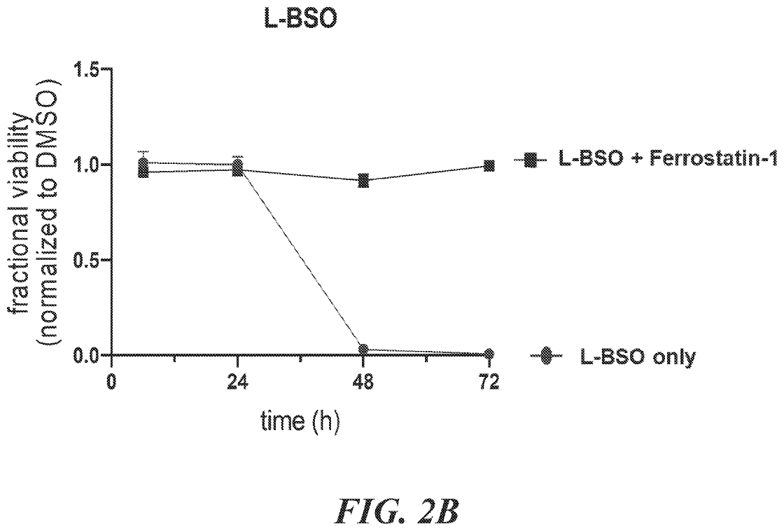

A drug delivery system was applied to a: solid tumor animal model. Animals were administered (1) L-BSO or (2) L-BSO+liproxstatin-1 (lip-1) as a ferroptosis-rescue agent. Drugs were loaded into an implantable drug delivery system to achieve concentrations of 1-10 μM for both BSO+lip-1 at the tumor site. After 24 hours, tumors were removed and stained for cleaved caspase-3 to indicate cell death in the tumor (FIG. 2A). The BSO-treated tumor section shows the recruitment of white blood cells to the tumor indicating immune cell recruitment and cell death at the tumor site. Tumors treated with BSO also exhibited significant reductions in fractional viability as compared with BSO+fer-1 treated sections of the tumor (FIG. 2B). Therefore, ferroptosis can be induced by both local and systemic administration of BSO.

### Example 9: Ferroptosis Induction by Sustained, Targeted Administration of ML-210

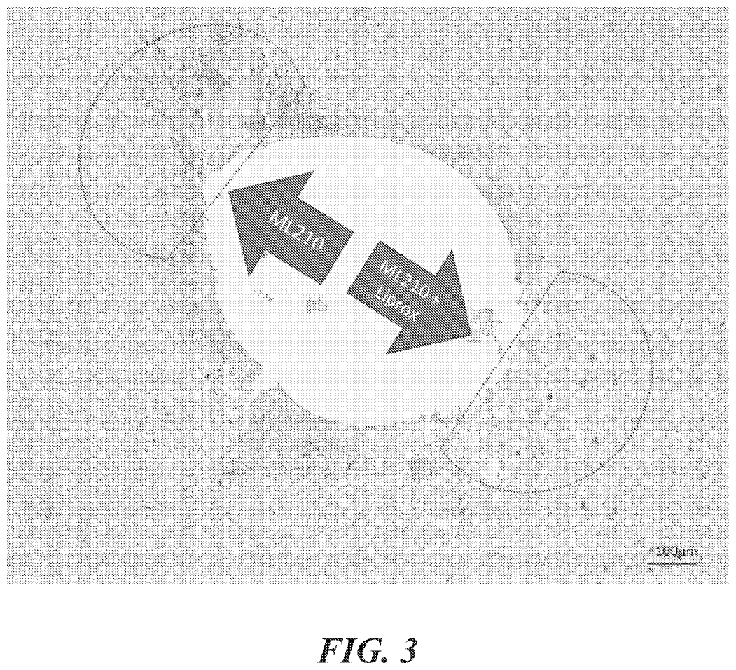

A drug delivery system was applied to a: solid tumor animal model. Animals were administered (1) ML-210 or (2) ML-210+liproxstatin-1 (lip-1), as a ferroptosis rescue agent. Drugs were loaded into an implantable drug delivery system to achieve concentrations of 1-10 μM for both ML-210 and ML-210+liproxstatin-1 (lip-1) at the tumor site. After 24 hours, tumors were removed and stained for cleaved caspase-3. The ML-210 treated tumor section shows the recruitment of white blood cells to the tumor indicating immune cell recruitment and cell death at the tumor site (FIG. 3).

### Example 10: Ferroptosis Induction by Administration of RSL3

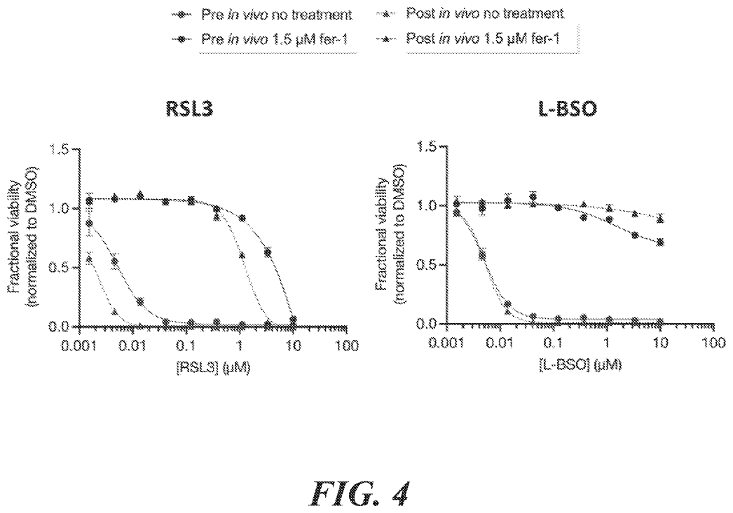

Dose-response curves for BSO and RSL3 are shown in FIG. 4 with and without anti-ferroptosis rescue agent treatment (indicated as no treatment or +1.5 μM fer-1). Ferrostatin-1 prevents cell death in RSL3 and BSO treated tumors in vivo. Furthermore, post-in vivo treatment with RSL3 and fer-1 showed a reduction in the fractional viability of cancer cells that was not observed with BSO+fer-1, indicating that RSL3 is a robust inducer of ferroptosis.

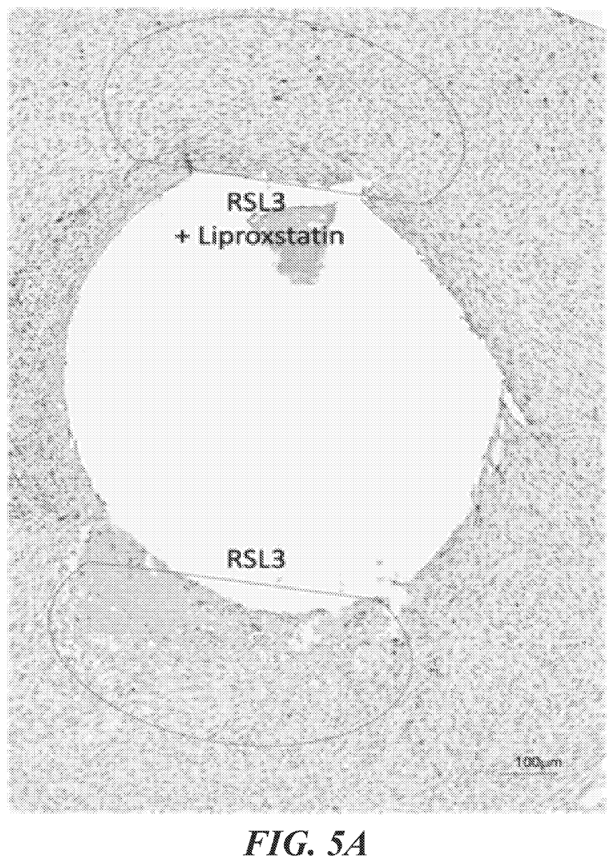

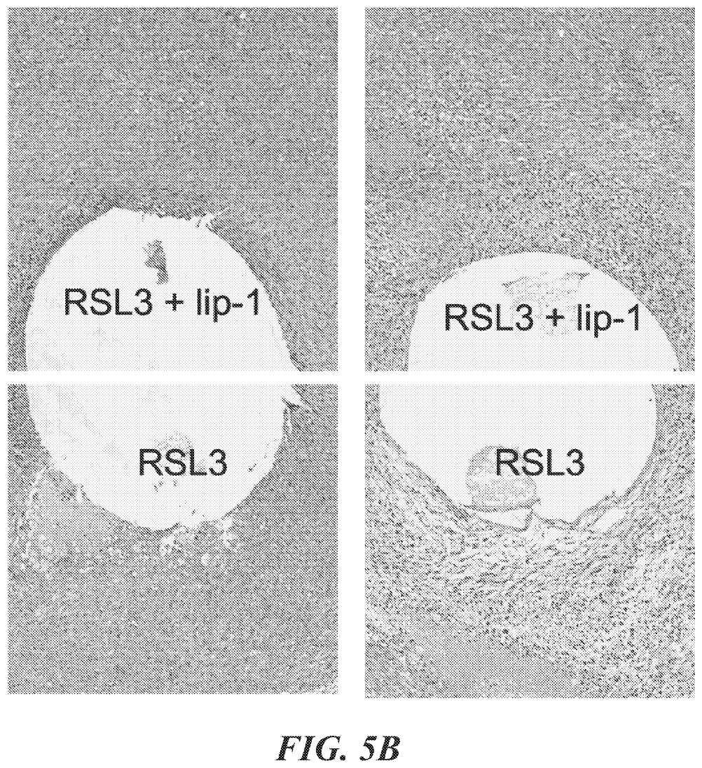

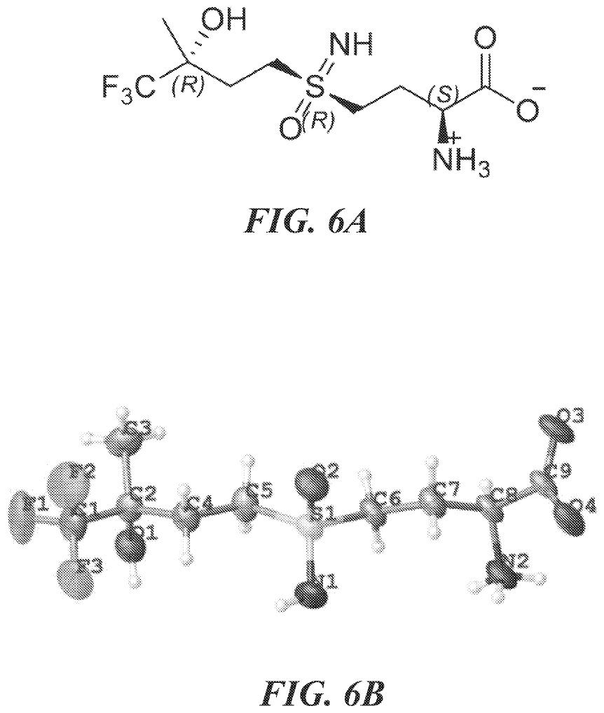

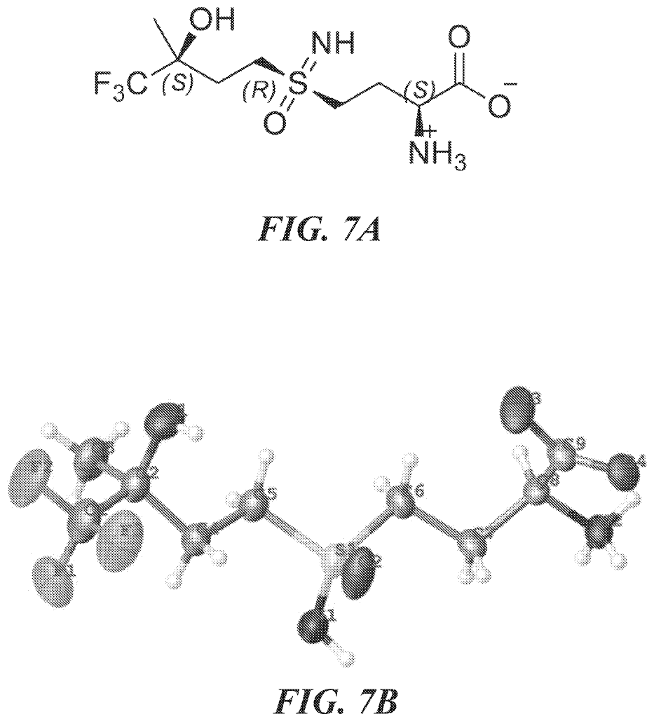

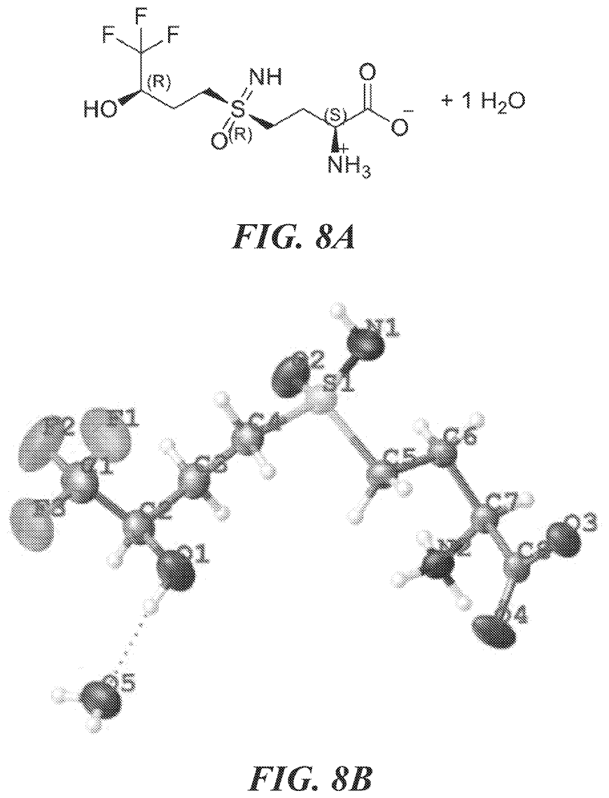

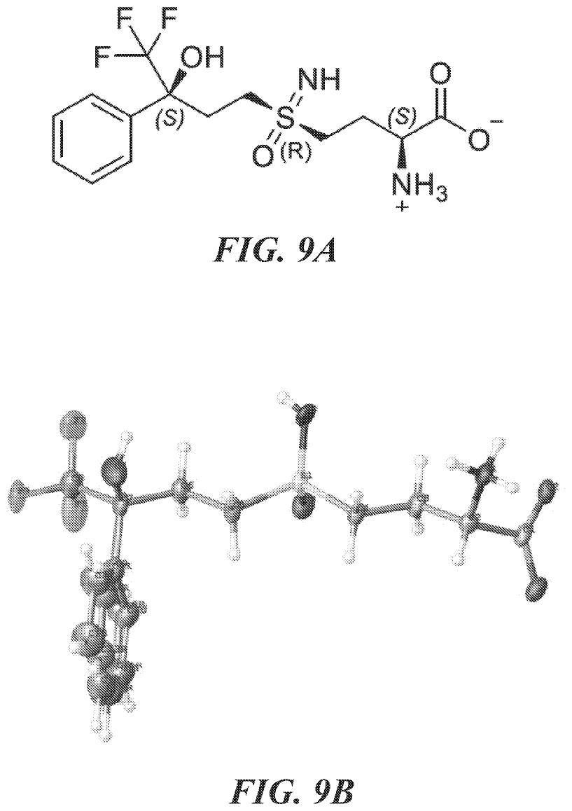

To achieve therapeutic doses of RSL3 in vivo, a drug delivery system was applied to a: solid tumor animal model. Animals were administered (1) RSL3 or (2) RSL3+liproxstatin (lip-1) as a ferroptosis rescue agent. Drugs were loaded into an implantable drug delivery system to achieve concentrations of 1-10 μM for both RSL-3 and RSL3 and lip-1 co-administration at the tumor site. After 24 hours of sustained administration, tumors were removed and stained for cleaved caspase-3 (FIG. 5A). Dashed lines indicate region of drug exposure. FIG. 5B shows representative H&E images at 18 hrs post treatment with (1) RSL-3 and or (2) RSL-3 and lip-las indicated. White blood cell recruitment and cell death were prominent in RSL3 treated tumor sections as compared with RSL3+lip-1 treated tumor sections.

### Example 11: System for In Vivo Ferroptosis-Inducing Agent Delivery

Ferroptosis-inducing agents and/or priming agents are administered systemically by injection to a mammal to establish local pharmacokinetics for the drugs. Representative drugs are tested include: BSO, RSL3, ML-210 and lip-1. Representative animal models that can be used include for instance, those harboring tumors in a flammable membrane state.

A drug delivery system with microwells is loaded with approximately 1.5 micrograms of a ferroptosis-inducing agent (crystalline powder) per microwell. The system is loaded with the same drugs based on the results of the systemic testing. Each drug is loaded separately and in more than one concentration, as well as in combination. After 10, 12, 18, 24, 36 or 48 hours, devices are removed and histology of the tissue was examined to determine the effect of the ferroptosis-inducing agents on the tumor cells adjacent to each well. The effects of compounds eluted from microwells are assessed by different techniques. Tissue excised with the device is assayed by standard histopathological techniques, including immunohistochemistry and immunofluorescence. Ingrowth of tissue, ranging from 20 to about 300 microns, are visualized by staining tissue/device section by standard immuno-histochemistry (IHC) techniques, including hematoxylin & eosin (H&E) staining, or any nuclear cell stain such as DAPI. Mass spectrometry is used to measure local biomarkers indicative of an effect of a ferroptosis-inducing agent (e.g., mesenchymal cell state markers or PUFA concentration). Analysis for apoptosis, necrosis, mitotic cell death, and proliferation is conducted. The local microdose response is determined and used to define an appropriate therapeutic regime for the cancer.

Several methods for controlling the release/diffusion of ferroptosis-inducing agents into tissue, including precise spatial placement of microwells along device mantle; geometry and size of microwells; and formulation of released agents are developed. The device microwells from which the ferroptosis-inducing agents diffuse are engineered to expose only regions of tissue that are directly adjacent to the microwell opening, to the agent that is being released. This creates distinct local regions in the tissue in which the effects of compounds are assessed without interference of other compounds released from different microwells. Creation of discrete areas of drug are useful to assess the efficacy of the different agents, or combinations thereof, and/or dosages and/or times of release (sustained, pulsed, delayed, bolus followed by sustained, etc.).

Agents are released upward and diffused into a larger region, or released downward into a relatively smaller region of a target tissue. The precise control over the transport time as a function of distance from microwells provide a local concentration of a first agent as a function of distance from the microwell, at multiple time points following in vivo implantation.

Concentration gradient regions are defined as the distance from the microwell increases, the concentration of the agent being administered decreases. Cleaved caspase 3 positive cells as percent area of 3, 3′-diaminobenzidine (DAB) staining as a function of distance from the microwell is one example of a functional readout from the implanted drug delivery system. The agent concentration gradient is formed approximately 100-250 m from the microwell with tissue concentration as greatest in the regions closest to the microwell.

The system is used to deliver a microdose of a ferroptosis-inducing agent to a tissue in vivo. The system is also used to deliver a priming agent (e.g., lip-1), followed by a ferroptosis-inducing agent (e.g., RSL3)) to a tissue in vivo to induce targeted cell death in the tissue. The system is also implanted directly into tumor of about 6 millimeters (mm) to about 7 mm in diameter to achieve a minimum amount of about 10 ng/mm² of drug at the site of the microwell for at least 4 hours.

### Example 12: X-Ray Crystallography of Compound 322

A 30 mg sample of Compound 322 and 300 μL ammonium hydroxide (lmol/L) were dissolved in 2 mL H₂O, then the solution was freeze-dried. The dry sample was dissolved in 1.3 mL methanol/H₂O (12:1) and kept in a half sealed 4 mL vial. The solution evaporated slowly at 45° C. Crystals were observed in the second day. The crystal was a colourlessneedle with the following dimensions: 0.30×0.10×0.04 mm3. The symmetry of the crystal structure was assigned the monoclinic space group C₂ with the following parameters: a=20.6265 (6) Å, b=5.7439 (2) Å, c=34.6420 (14) Å, α=90°, β=96.425 (3)°, γ=90°, V=4078.5 (2) Å3, Z=12, Dc=1.497 g/cm3, F(000)=1920.0, μ(CuKα)=2.603 mm-1, and T=293 (2) K.

A total of 29208 reflections were collected in the 2θ range from 5.134 to 133.17. The limiting indices were: −19≤h≤24, −6≤k≤6, −41≤1≤41; which yielded 7048 unique reflections (Rint=0.1188). Data was collected using a Rigaku Oxford Diffraction XtaLAB Synergy-S four-circle diffractometer equipped with a HyPix-6000HE area detector. The cryogenic system used was Oxford Cryostream 800. Cu radiation was used (λ=1.54184 Å, 50 W, Micro focus source with multilayer mirror (μ-CMF)) with a d=35 mm crystal distance from the detector (Tube Voltage=50 kV; Tube Current 1 mA). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 536, compared with 7048 data. All reflections were included in the refinement. The goodness of fit on F² was 1.097 with a final R value for [I>2σ (I)]R1=0.0922 and wR2=0.2195. The largest differential peak and hole were 0.64 and −0.52 Å-3, respectively.

TABLE 23
Summary of crystallographic data for compound 322.

| Crystal size/mm3 | 0.30 × 0.10 × 0.04 | |
|---|---|---|
| Radiation Type | CuKα | (λ = 1.54184) |
| Crystal system | monoclinic | |
| Space group | C2 | |
| a/Å | 20.6265 | (6) |
| b/Å | 5.7439 | (2) |
| c/Å | 34.6420 | (14) |
| α/° | 90 | |
| β/° | 96.425 | (3) |
| γ/° | 90 | |
| Cell Volume/Å3 | 4078.5 | (2) |
| Cell Formula Units Z | 12 | |
| Crystal Density calc g/cm3 | 1.497 | |
| Crystal F(000) | 1920.0 | |
| Absorption Coefficient μ/mm−1 | 2.603 | |
| Index ranges | −19 ≤ h ≤ 24, −6 ≤ k ≤ 6, −41 ≤ | |
| | l ≤ 41 | |
| Cell Measurement Temperature/K | 293 | (2) |
| 2θ range for data collection/° | 5.134 to 133.17 | |
| Goodness-of-fit on F2 | 1.097 | |
| Final R indexes [I >= 2σ (I)] | R1 = 0.0922, wR2 = 0.2195 | |
| Final R indexes [all data] | R1 = 0.1029, wR2 = 0.2228 | |
| Largest diff. peak/hole/e Å−3 | 0.64/−0.52 | |
| Reflections collected/unique | 29208/7048 | [Rint = 0.1188] |
| Flack parameter | 0.13 | (5) |

| | |
|---|---|
| BASF | 0.13 |

TABLE 24
Atomic Coordinates (×10\hat{ }4) and equivalent isotropic displacement
parameters (A\hat{ }2 × 10\hat{ }3) for X-Ray Crystal Structure of Compound 322

| Atom | | x | | y | | z | | U(eq) | |
|---|---|---|---|---|---|---|---|---|---|
| S | (1) | 4884.7 | (10) | 4508 | (4) | 7182.1 | (6) | 32.6 | (5) |
| S | (1A) | 1735 | (1) | 5132 | (4) | 6478.5 | (7) | 33.6 | (5) |
| S | (1B) | 2928.0 | (14) | 2479 | (6) | 9018.6 | (9) | 61.2 | (8) |
| F | (2) | 3410 | (3) | 1489 | (18) | 5893 | (3) | 87 | (2) |
| O | (2) | 4547 | (3) | 6663 | (13) | 7050 | (2) | 48.4 | (16) |
| O | (1) | 4799 | (3) | −1549 | (13) | 6342 | (2) | 47.2 | (16) |
| O | (4B) | 4047 | (3) | 6812 | (12) | 8267 | (2) | 51.3 | (18) |
| F | (2A) | 691 | (4) | 2410 | (20) | 5058 | (2) | 94 | (2) |
| N | (1A) | 1262 | (4) | 3895 | (15) | 6713 | (2) | 42.4 | (18) |
| N | (2A) | 2958 | (4) | 5960 | (13) | 7711 | (3) | 40.1 | (17) |
| F | (1A) | 1178 | (5) | −701 | (18) | 4905 | (2) | 99 | (3) |
| C | (9) | 6342 | (4) | 10010 | (15) | 8103 | (3) | 35.9 | (16) |
| N | (2) | 6345 | (4) | 5825 | (12) | 8243 | (3) | 41.2 | (18) |
| O | (4) | 6062 | (4) | 10325 | (13) | 8393 | (2) | 51.4 | (17) |
| C | (8) | 6327 | (4) | 7581 | (15) | 7922 | (3) | 34.7 | (15) |
| C | (9A) | 3026 | (4) | 10154 | (16) | 7558 | (3) | 38.0 | (16) |
| O | (1A) | 1974 | (4) | −739 | (14) | 5603 | (2) | 60.3 | (19) |
| O | (3) | 6633 | (3) | 11533 | (11) | 7925 | (2) | 47.5 | (17) |
| O | (3B) | 5073 | (3) | 5589 | (13) | 8441 | (3) | 59 | (2) |
| O | (2A) | 1547 | (3) | 7361 | (13) | 6307 | (2) | 50.5 | (16) |
| F | (3) | 3435 | (4) | −1346 | (17) | 6299 | (2) | 86 | (2) |
| N | (2B) | 4675 | (4) | 1139 | (14) | 8261 | (3) | 45.3 | (19) |
| O | (3A) | 3207 | (4) | 11685 | (12) | 7346 | (3) | 56.3 | (19) |
| C | (8A) | 2993 | (4) | 7687 | (15) | 7387 | (3) | 30.6 | (14) |
| F | (1) | 3790 | (4) | −1827 | (18) | 5750 | (2) | 93 | (3) |
| C | (7B) | 4197 | (5) | 2280 | (20) | 8854 | (3) | 57 | (2) |
| C | (2) | 4441 | (4) | 442 | (18) | 6212 | (3) | 37.1 | (14) |
| F | (3A) | 623 | (5) | −660 | (20) | 5393 | (2) | 115 | (3) |
| C | (7A) | 2401 | (4) | 7463 | (16) | 7084 | (3) | 37.0 | (18) |
| C | (3A) | 2069 | (5) | 2670 | (20) | 5223 | (3) | 51 | (2) |
| C | (5) | 5011 | (4) | 2920 | (20) | 6750 | (3) | 43 | (2) |
| C | (4A) | 1410 | (4) | 2760 | (20) | 5802 | (3) | 43 | (2) |
| C | (9B) | 4463 | (4) | 5314 | (17) | 8364 | (3) | 38.2 | (18) |
| O | (2B) | 3006 | (6) | 350 | (20) | 9249 | (4) | 96 | (3) |
| C | (4) | 4357 | (4) | 2097 | (18) | 6548 | (3) | 39.1 | (18) |
| C | (3) | 4817 | (5) | 1520 | (20) | 5895 | (3) | 52 | (2) |
| N | (1) | 4588 | (4) | 3045 | (17) | 7482 | (3) | 49 | (2) |
| C | (7) | 5709 | (5) | 7331 | (17) | 7653 | (3) | 41.1 | (19) |
| C | (6A) | 2454 | (4) | 5478 | (18) | 6797 | (3) | 39.1 | (18) |
| C | (2A) | 1630 | (4) | 1316 | (18) | 5464 | (3) | 38.4 | (14) |
| C | (6B) | 3719 | (6) | 3730 | (20) | 9052 | (4) | 66 | (3) |
| C | (1) | 3781 | (5) | −380 | (20) | 6038 | (3) | 53.2 | (16) |
| O | (4A) | 2875 | (5) | 10373 | (14) | 7894 | (3) | 66 | (2) |
| C | (4B) | 1804 | (8) | 3730 | (40) | 9318 | (5) | 94 | (4) |
| C | (8B) | 4223 | (5) | 2805 | (17) | 8418 | (3) | 40.9 | (17) |
| C | (5B) | 2478 | (7) | 4580 | (30) | 9260 | (4) | 81 | (3) |
| C | (5A) | 2005 | (4) | 3360 | (20) | 6096 | (3) | 43 | (2) |
| N | (1B) | 2603 | (5) | 2420 | (30) | 8616 | (3) | 82 | (3) |
| C | (1A) | 1027 | (6) | 570 | (30) | 5207 | (3) | 60.9 | (18) |
| C | (6) | 5688 | (4) | 5209 | (17) | 7386 | (3) | 38.8 | (18) |
| C | (2B) | 1372 | (10) | 5780 | (40) | 9463 | (5) | 112 | (4) |
| F | (3B) | 441 | (7) | 3430 | (40) | 9218 | (5) | 200 | (7) |
| O | (1B) | 1205 | (8) | 7360 | (30) | 9143 | (4) | 131 | (4) |
| F | (1B) | 327 | (7) | 6250 | (40) | 9631 | (5) | 215 | (7) |
| F | (2B) | 826 | (8) | 3120 | (40) | 9824 | (5) | 220 | (8) |
| C | (3B) | 1712 | (12) | 7440 | (50) | 9774 | (6) | 149 | (7) |
| C | (1B) | 757 | (11) | 4570 | (60) | 9537 | (7) | 142 | (5) |

TABLE 25
Bond Lengths for X-Ray Crystal Structure of Compound 322

| Atom | Atom | Length/Å | Atom | Atom | Length/Å |
|---|---|---|---|---|---|
| S(1) | O(2) | 1.468(7) | O(3B) | C(9B) | 1.267(12) |
| S(1) | C(5) | 1.795(10) | F(3) | C(1) | 1.332(14) |
| S(1) | N(1) | 1.517(9) | N(2B) | C(8B) | 1.482(13) |
| S(1) | C(6) | 1.773(9) | C(8A) | C(7A) | 1.524(12) |
| S(1A) | N(1A) | 1.515(8) | F(1) | C(1) | 1.301(14) |
| S(1A) | O(2A) | 1.446(7) | C(7B) | C(6B) | 1.512(17) |
| S(1A) | C(6A) | 1.757(9) | C(7B) | C(8B) | 1.545(15) |
| S(1A) | C(5A) | 1.808(10) | C(2) | C(4) | 1.528(13) |
| S(1B) | O(2B) | 1.458(12) | C(2) | C(3) | 1.544(14) |
| S(1B) | C(6B) | 1.775(12) | C(2) | C(1) | 1.502(13) |
| S(1B) | C(5B) | 1.788(15) | F(3A) | C(1A) | 1.316(14) |
| S(1B) | N(1B) | 1.478(12) | C(7A) | C(6A) | 1.524(13) |
| F(2) | C(1) | 1.382(15) | C(3A) | C(2A) | 1.512(14) |
| O(1) | C(2) | 1.408(12) | C(5) | C(4) | 1.523(13) |
| O(4B) | C(9B) | 1.235(12) | C(4A) | C(2A) | 1.544(14) |
| F(2A) | C(1A) | 1.337(17) | C(4A) | C(5A) | 1.544(13) |
| N(2A) | C(8A) | 1.505(12) | C(9B) | C(8B) | 1.542(13) |
| F(1A) | C(1A) | 1.340(15) | C(7) | C(6) | 1.528(13) |
| C(9) | O(4) | 1.228(12) | C(2A) | C(1A) | 1.509(14) |
| C(9) | C(8) | 1.527(12) | C(4B) | C(5B) | 1.51(2) |
| C(9) | O(3) | 1.258(11) | C(4B) | C(2B) | 1.59(2) |
| N(2) | C(8) | 1.496(12) | C(2B) | O(1B) | 1.44(2) |
| C(8) | C(7) | 1.502(13) | C(2B) | C(3B) | 1.55(3) |
| C(9A) | O(3A) | 1.231(13) | C(2B) | C(1B) | 1.50(4) |
| C(9A) | C(8A) | 1.534(12) | F(3B) | C(1B) | 1.38(3) |
| C(9A) | O(4A) | 1.247(13) | F(1B) | C(1B) | 1.37(3) |
| O(1A) | C(2A) | 1.433(13) | F(2B) | C(1B) | 1.29(3) |

TABLE 26
Bond Angles for X-Ray Crystal Structure of Compound 322

| Atom | Atom | Atom | Angle/° | Atom | Atom | Atom | Angle/° |
|---|---|---|---|---|---|---|---|
| O(2) | S(1) | C(5) | 106.1(5) | O(3B) | C(9B) | C(8B) | 114.6(8) |
| O(2) | S(1) | N(1) | 117.8(5) | C(5) | C(4) | C(2) | 112.0(8) |
| O(2) | S(1) | C(6) | 108.8(5) | C(8) | C(7) | C(6) | 114.7(8) |
| N(1) | S(1) | C(5) | 113.3(6) | C(7A) | C(6A) | S(1A) | 112.2(6) |
| N(1) | S(1) | C(6) | 106.5(5) | O(1A) | C(2A) | C(3A) | 107.7(8) |
| C(6) | S(1) | C(5) | 103.3(5) | O(1A) | C(2A) | C(4A) | 111.3(8) |
| N(1A) | S(1A) | C(6A) | 105.3(5) | O(1A) | C(2A) | C(1A) | 107.9(10) |
| N(1A) | S(1A) | C(5A) | 113.5(5) | C(3A) | C(2A) | C(4A) | 112.9(9) |
| O(2A) | S(1A) | N(1A) | 118.4(5) | C(1A) | C(2A) | C(3A) | 109.0(9) |
| O(2A) | S(1A) | C(6A) | 109.1(5) | C(1A) | C(2A) | C(4A) | 107.9(8) |
| O(2A) | S(1A) | C(5A) | 106.7(5) | C(7B) | C(6B) | S(1B) | 112.9(9) |
| C(6A) | S(1A) | C(5A) | 102.5(4) | F(2) | C(1) | C(2) | 109.9(10) |
| O(2B) | S(1B) | C(6B) | 104.9(7) | F(3) | C(1) | F(2) | 104.8(9) |
| O(2B) | S(1B) | C(5B) | 109.9(7) | F(3) | C(1) | C(2) | 112.9(9) |
| O(2B) | S(1B) | N(1B) | 120.5(9) | F(1) | C(1) | F(2) | 105.7(9) |
| C(6B) | S(1B) | C(5B) | 102.6(7) | F(1) | C(1) | F(3) | 108.2(11) |
| N(1B) | S(1B) | C(6B) | 112.8(7) | F(1) | C(1) | C(2) | 114.7(9) |
| N(1B) | S(1B) | C(5B) | 104.6(7) | C(5B) | C(4B) | C(2B) | 111.1(17) |
| O(4) | C(9) | C(8) | 118.6(8) | N(2B) | C(8B) | C(7B) | 109.0(8) |
| O(4) | C(9) | O(3) | 126.3(9) | N(2B) | C(8B) | C(9B) | 109.5(7) |
| O(3) | C(9) | C(8) | 115.1(8) | C(9B) | C(8B) | C(7B) | 110.4(9) |
| N(2) | C(8) | C(9) | 108.4(8) | C(4B) | C(5B) | S(1B) | 112.2(12) |
| N(2) | C(8) | C(7) | 110.2(7) | C(4A) | C(5A) | S(1A) | 108.8(6) |
| C(7) | C(8) | C(9) | 108.6(8) | F(2A) | C(1A) | F(1A) | 106.6(10) |
| O(3A) | C(9A) | C(8A) | 115.6(9) | F(2A) | C(1A) | C(2A) | 111.0(11) |
| O(3A) | C(9A) | O(4A) | 127.7(9) | F(1A) | C(1A) | C(2A) | 111.5(10) |
| O(4A) | C(9A) | C(8A) | 116.7(9) | F(3A) | C(1A) | F(2A) | 106.6(12) |
| N(2A) | C(8A) | C(9A) | 109.0(8) | F(3A) | C(1A) | F(1A) | 107.9(11) |
| N(2A) | C(8A) | C(7A) | 110.9(7) | F(3A) | C(1A) | C(2A) | 112.9(10) |
| C(7A) | C(8A) | C(9A) | 109.9(7) | C(7) | C(6) | S(1) | 112.5(7) |
| C(6B) | C(7B) | C(8B) | 115.5(10) | O(1B) | C(2B) | C(4B) | 108.5(14) |
| O(1) | C(2) | C(4) | 111.2(8) | O(1B) | C(2B) | C(3B) | 101.6(19) |
| O(1) | C(2) | C(3) | 105.5(8) | O(1B) | C(2B) | C(1B) | 106.8(19) |
| O(1) | C(2) | C(1) | 107.0(9) | C(3B) | C(2B) | C(4B) | 116.7(16) |
| C(4) | C(2) | C(3) | 113.6(9) | C(1B) | C(2B) | C(4B) | 103(2) |
| C(1) | C(2) | C(4) | 109.1(8) | C(1B) | C(2B) | C(3B) | 119.5(19) |
| C(1) | C(2) | C(3) | 110.2(8) | F(3B) | C(1B) | C(2B) | 114.6(17) |
| C(8A) | C(7A) | C(6A) | 113.7(7) | F(1B) | C(1B) | C(2B) | 107(3) |
| C(4) | C(5) | S(1) | 109.8(7) | F(1B) | C(1B) | F(3B) | 105(2) |
| C(2A) | C(4A) | C(5A) | 110.0(7) | F(2B) | C(1B) | C(2B) | 114(2) |
| O(4B) | C(9B) | O(3B) | 127.8(9) | F(2B) | C(1B) | F(3B) | 108(3) |
| O(4B) | C(9B) | C(8B) | 117.5(8) | F(2B) | C(1B) | F(1B) | 106.9(17) |

TABLE 27
Hydrogen Bonds for X-Ray Crystal Structure of Compound 322

| | | | d(D − | d(H − | d(D − | D − H − |
|---|---|---|---|---|---|---|
| D | H | A | H)/Å | A)/Å | A)/Å | A/° |
| O(1) | H(1) | O(2) ¹ | 0.82 | 1.99 | 2.762(11) | 157.6 |
| N(2A) | H(2AA) | O(3A) ¹ | 0.89 | 1.96 | 2.835(11) | 169.4 |
| N(2A) | H(2AB) | O(3) ² | 0.89 | 2.05 | 2.931(11) | 171.9 |
| N(2A) | H(2AC) | O(4B) | 0.89 | 1.96 | 2.835(11) | 169.4 |
| N(2) | H(2A) | O(3) ¹ | 0.89 | 1.90 | 2.790(10) | 174.5 |
| N(2) | H(2B) | O(3B) | 0.89 | 1.92 | 2.789(11) | 166.8 |
| N(2) | H(2C) | N(1B) ³ | 0.89 | 2.10 | 2.913(14) | 152.4 |
| O(1A) | H(1AB) | O(2A) ¹ | 0.82 | 2.08 | 2.898(11) | 173.6 |
| N(2B) | H(2BA) | N(1) | 0.89 | 2.05 | 2.898(13) | 160.3 |
| N(2B) | H(2BB) | O(4B) ¹ | 0.89 | 1.92 | 2.804(10) | 173.4 |
| N(2B) | H(2BC) | O(4) ¹ | 0.89 | 2.14 | 2.884(11) | 140.7 |
| N(1) | H(1A) | O(3A) ¹ | 0.877(13) | 2.078(13) | 2.939(11) | 167(7) |
| N(1B) | H(1B) | O(4A) ¹ | 0.878(14) | 2.020(14) | 2.874(14) | 164(7) |
| N(1A) | H(1AA) | O(1) ⁴ | 0.874(13) | 2.295(13) | 3.155(10) | 168(7) |

¹ +X, −1 + Y, +Z;
² −1/2 + X, −1/2 + Y, +Z;
³ 1/2 + X, 1/2 + Y, +Z;
⁴ −1/2 + X, 1/2 + Y, +Z

TABLE 28
Torsion Angles for X-Ray Crystal Structure of Compound 322

| A | B | C | D | Angle/° | A | B | C | D | Angle/° |
|---|---|---|---|---|---|---|---|---|---|
| S(1) | C(5) | C(4) | C(2) | −171.7(7) | C(4) | C(2) | C(1) | F(2) | 61.6(11) |
| O(2) | S(1) | C(5) | C(4) | −68.0(9) | C(4) | C(2) | C(1) | F(3) | −54.9(14) |
| O(2) | S(1) | C(6) | C(7) | 44.1(8) | C(4) | C(2) | C(1) | F(1) | −179.5(10) |
| O(1) | C(2) | C(4) | C(5) | 59.7(11) | C(3) | C(2) | C(4) | C(5) | −59.1(11) |
| O(1) | C(2) | C(1) | F(2) | −178.1(8) | C(3) | C(2) | C(1) | F(2) | −63.9(12) |
| O(1) | C(2) | C(1) | F(3) | 65.4(12) | C(3) | C(2) | C(1) | F(3) | 179.6(10) |
| O(1) | C(2) | C(1) | F(1) | −59.1(13) | C(3) | C(2) | C(1) | F(1) | 55.1(14) |
| O(4B) | C(9B) | C(8B) | N(2B) | 140.2(9) | N(1) | S(1) | C(5) | C(4) | 62.8(9) |
| O(4B) | C(9B) | C(8B) | C(7B) | −99.9(11) | N(1) | S(1) | C(6) | C(7) | −83.8(8) |
| N(1A) | S(1A) | C(6A) | C(7A) | −77.4(8) | C(6A) | S(1A) | C(5A) | C(4A) | 176.3(8) |
| N(1A) | S(1A) | C(5A) | C(4A) | 63.2(9) | C(2A) | C(4A) | C(5A) | S(1A) | 178.0(8) |
| N(2A) | C(8A) | C(7A) | C(6A) | −80.7(9) | C(6B) | S(1B) | C(5B) | C(4B) | −172.0(12) |
| C(9) | C(8) | C(7) | C(6) | 167.5(8) | C(6B) | C(7B) | C(8B) | N(2B) | −176.0(9) |
| N(2) | C(8) | C(7) | C(6) | −73.9(10) | C(6B) | C(7B) | C(8B) | C(9B) | 63.6(12) |
| O(4) | C(9) | C(8) | N(2) | −34.8(11) | C(1) | C(2) | C(4) | C(5) | 177.5(10) |
| O(4) | C(9) | C(8) | C(7) | 84.9(11) | O(4A) | C(9A) | C(8A) | N(2A) | −15.4(11) |
| C(8) | C(7) | C(6) | S(1) | 162.5(7) | O(4A) | C(9A) | C(8A) | C(7A) | 106.3(10) |
| C(9A) | C(8A) | C(7A) | C(6A) | 158.8(8) | C(4B) | C(2B) | C(1B) | F(3B) | −58(3) |
| O(1A) | C(2A) | C(1A) | F(2A) | −178.4(9) | C(4B) | C(2B) | C(1B) | F(1B) | −174.6(17) |
| O(1A) | C(2A) | C(1A) | F(1A) | −59.6(12) | C(4B) | C(2B) | C(1B) | F(2B) | 67(3) |
| O(1A) | C(2A) | C(1A) | F(3A) | 62.1(14) | C(8B) | C(7B) | C(6B) | S(1B) | 88.7(12) |
| O(3) | C(9) | C(8) | N(2) | 147.3(8) | C(5B) | S(1B) | C(6B) | C(7B) | −178.2(10) |
| O(3) | C(9) | C(8) | C(7) | −93.0(10) | C(5B) | C(4B) | C(2B) | O(1B) | 72(2) |
| O(3B) | C(9B) | C(8B) | N(2B) | −42.4(12) | C(5B) | C(4B) | C(2B) | C(3B) | −42(2) |
| O(3B) | C(9B) | C(8B) | C(7B) | 77.6(11) | C(5B) | C(4B) | C(2B) | C(1B) | −174.9(17) |
| O(2A) | S(1A) | C(6A) | C(7A) | 50.8(8) | C(5A) | S(1A) | C(6A) | C(7A) | 163.7(7) |
| O(2A) | S(1A) | C(5A) | C(4A) | −69.1(8) | C(5A) | C(4A) | C(2A) | O(1A) | 56.0(11) |
| O(3A) | C(9A) | C(8A) | N(2A) | 163.7(8) | C(5A) | C(4A) | C(2A) | C(3A) | −65.2(12) |
| O(3A) | C(9A) | C(8A) | C(7A) | −74.6(10) | C(5A) | C(4A) | C(2A) | C(1A) | 174.3(10) |
| C(8A) | C(7A) | C(6A) | S(1A) | 175.1(7) | N(1B) | S(1B) | C(6B) | C(7B) | −66.2(12) |
| C(3A) | C(2A) | C(1A) | F(2A) | −61.7(13) | N(1B) | S(1B) | C(5B) | C(4B) | 70.0(14) |
| C(3A) | C(2A) | C(1A) | F(1A) | 57.0(14) | C(6) | S(1) | C(5) | C(4) | 177.6(8) |
| C(3A) | C(2A) | C(1A) | F(3A) | 178.8(12) | C(2B) | C(4B) | C(5B) | S(1B) | −169.9(12) |
| C(5) | S(1) | C(6) | C(7) | 156.5(7) | O(1B) | C(2B) | C(1B) | F(3B) | 56(3) |
| C(4A) | C(2A) | C(1A) | F(2A) | 61.3(12) | O(1B) | C(2B) | C(1B) | F(1B) | −60(2) |
| C(4A) | C(2A) | C(1A) | F(1A) | 180.0(10) | O(1B) | C(2B) | C(1B) | F(2B) | −179(2) |
| C(4A) | C(2A) | C(1A) | F(3A) | −58.3(15) | C(3B) | C(2B) | C(1B) | F(3B) | 171(2) |
| O(2B) | S(1B) | C(6B) | C(7B) | 66.9(11) | C(3B) | C(2B) | C(1B) | F(1B) | 54(3) |
| O(2B) | S(1B) | C(5B) | C(4B) | −60.8(14) | C(3B) | C(2B) | C(1B) | F(2B) | −64(3) |

### Example 13: X-Ray Crystallography of Compound 324

A 10 mg sample of compound 324 was dissolved in 100 L H2O and placed in the inner 4 mL vial (no lid). The outer 40 mL vial was covered with 4 mL acetonitrile. The outer vial was sealed and the vapor was allowed to spread slowly. Crystals were observed in the third day. The crystal was a colourless needle with the following dimensions: 0.30×0.10×0.05 mm3. The symmetry of the crystal structure was assigned the monoclinic space group P21with the following parameters: a=7.48980 (10) Å, b=5.73240 (10) Å, c=15.8666 (3) Å, α=90°, β=99.366 (2°), γ=90°, V=672.14 (2) Å3, Z=2, Dc=1.513 g/cm3, F(000)=320.0, μ(CuKα)=2.632 mm-1, and T=293 (2) K. A total of 14194 reflections were collected in the 2θ range from 5.646 to 133.19. The limiting indices were: −8≤h≤8, −6≤k≤5, −18≤1≤18; which yielded 2162 unique reflections (Rint=0.0442). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 179, compared with 2162 data. All reflections were included in the refinement. The goodness of fit on F² was 1.094 with a final R value for [I>2σ (I)]R1=0.0430 and wR2=0.1151. The largest differential peak and holewere 0.49 and −0.19 Å-3, respectively. Data was collected using a Rigaku Oxford Diffraction XtaLAB Synergy-S four-circle diffractometer equipped with a HyPix-6000HE area detector. The cryogenic system used was Oxford Cryostream 800. Cu radiation was used (λ=1.54184 Å, 50 W, Micro focus source with multilayer mirror (μ-CMF)) with a d=35 mm crystal distance from the detector (Tube Voltage=50 kV; Tube Current 1 mA).

TABLE 29
Summary of crystallographic data for compound 324.

| Crystal size/mm3 | 0.30 × 0.10 × 0.05 | |
|---|---|---|
| Radiation Type | CuKα | (λ = 1.54184) |
| Crystal system | monoclinic | |
| Space group | P21 | |
| a/Å | 7.48980 | (10) |
| b/Å | 5.73240 | (10) |
| c/Å | 15.8666 | (3) |
| α/° | 90 | |
| β/° | 99.366 | (2) |
| γ/° | 90 | |
| Cell Volume/Å3 | 672.14 | (2) |
| Cell Formula Units Z | 2 | |
| Crystal Density calc g/cm3 | 1.513 | |
| Crystal F(000) | 320.0 | |
| Absorption Coefficient μ/mm−1 | 2.632 | |
| Index ranges | −8 ≤ h ≤ 8, −6 ≤ k ≤ 5, −18 ≤ | |
| | l ≤ 18 | |
| Cell Measurement Temperature/K | 293 | (2) |
| 2θ range for data collection/° | 5.646 to 133.19 | |
| Goodness-of-fit on F2 | 1.094 | |
| Final R indexes [I >= 2σ (I)] | R1 = 0.0430, wR2 = 0.1151 | |
| Final R indexes [all data] | R1 = 0.0444, wR2 = 0.1164 | |
| Largest diff. peak/hole/e Å−3 | 0.49/−0.19 | |
| Reflections collected/unique | 14194/2162 | [Rint = 0.0442] |
| Flack parameter | −0.02 | (3) |

TABLE 30
Atomic Coordinates (×10\hat{ }4) and equivalent isotropic displacement
parameters (A\hat{ }2 × 10\hat{ }3) for X-Ray Crystal Structure of Compound 324

| Atom | | x | | y | | z | | U(eq) | |
|---|---|---|---|---|---|---|---|---|---|
| S | (1) | 3272.4 | (10) | 3245.0 | (17) | 7017.3 | (5) | 32.9 | (3) |
| O | (4) | −3216 | (3) | 4856 | (5) | 4900.2 | (17) | 40.9 | (6) |
| O | (2) | 3555 | (5) | 4813 | (7) | 6339.0 | (19) | 59.5 | (10) |
| O | (3) | −1988 | (5) | 6408 | (5) | 6149 | (2) | 60.8 | (10) |
| N | (2) | −3019 | (4) | 535 | (6) | 5467 | (2) | 33.7 | (7) |
| O | (1) | 5551 | (5) | 9080 | (6) | 8694 | (2) | 56.8 | (9) |
| F | (3) | 8646 | (5) | 8964 | (8) | 7946 | (2) | 89.7 | (13) |
| F | (1) | 9760 | (4) | 6157 | (9) | 8758 | (3) | 90.7 | (13) |
| F | (2) | 9227 | (5) | 9441 | (9) | 9292 | (3) | 101.3 | (14) |
| N | (1) | 4150 | (5) | 852 | (8) | 7072 | (2) | 48.5 | (10) |
| C | (8) | −2075 | (4) | 2330 | (6) | 6049 | (2) | 27.4 | (7) |
| C | (9) | −2470 | (4) | 4736 | (7) | 5658 | (3) | 33.2 | (8) |
| C | (4) | 6064 | (5) | 5337 | (8) | 8041 | (2) | 36.0 | (8) |
| C | (7) | −40 | (4) | 1802 | (7) | 6224 | (2) | 32.9 | (8) |
| C | (6) | 887 | (4) | 3148 | (9) | 7008 | (2) | 36.2 | (7) |
| C | (5) | 4104 | (5) | 4610 | (8) | 8011 | (2) | 35.9 | (8) |
| C | (2) | 6667 | (5) | 7089 | (8) | 8761 | (2) | 39.2 | (9) |
| C | (1) | 8579 | (6) | 7931 | (11) | 8694 | (3) | 58.9 | (13) |
| C | (3) | 6658 | (7) | 6052 | (11) | 9641 | (3) | 57.8 | (13) |

TABLE 31
Bond Lengths for X-Ray Crystal Structure of Compound 324

| Atom | | Atom | | Length/Å | | Atom | | Atom | | Length/Å | |
|---|---|---|---|---|---|---|---|---|---|---|---|
| S | (1) | O | (2) | 1.444 | (3) | F | (1) | C | (1) | 1.341 | (7) |
| S | (1) | N | (1) | 1.518 | (4) | F | (2) | C | (1) | 1.317 | (6) |
| S | (1) | C | (6) | 1.785 | (3) | C | (8) | C | (9) | 1.522 | (6) |
| S | (1) | C | (5) | 1.780 | (4) | C | (8) | C | (7) | 1.535 | (4) |
| O | (4) | C | (9) | 1.242 | (5) | C | (4) | C | (5) | 1.519 | (5) |
| O | (3) | C | (9) | 1.251 | (5) | C | (4) | C | (2) | 1.533 | (5) |
| N | (2) | C | (8) | 1.483 | (5) | C | (7) | C | (6) | 1.531 | (5) |
| O | (1) | C | (2) | 1.409 | (6) | C | (2) | C | (1) | 1.532 | (6) |
| F | (3) | C | (1) | 1.334 | (6) | C | (2) | C | (3) | 1.519 | (6) |

TABLE 32
Bond Angles for X-Ray Crystal Structure of Compound 324

| Atom | Atom | Atom | Angle/° | Atom | Atom | Atom | Angle/° |
|---|---|---|---|---|---|---|---|
| O(2) | S(1) | N(1) | 119.4(2) | C(7) | C(6) | S(1) | 110.1(2) |
| O(2) | S(1) | C(6) | 106.3(2) | C(4) | C(5) | S(1) | 110.2(3) |
| O(2) | S(1) | C(5) | 108.3(2) | O(1) | C(2) | C(4) | 112.1(3) |
| N(1) | S(1) | C(6) | 113.3(2) | O(1) | C(2) | C(1) | 106.8(4) |
| N(1) | S(1) | C(5) | 105.1(2) | O(1) | C(2) | C(3) | 107.1(4) |
| C(5) | S(1) | C(6) | 103.10(17) | C(1) | C(2) | C(4) | 108.7(3) |
| N(2) | C(8) | C(9) | 109.5(3) | C(3) | C(2) | C(4) | 112.6(4) |
| N(2) | C(8) | C(7) | 109.6(3) | C(3) | C(2) | C(1) | 109.4(4) |
| C(9) | C(8) | C(7) | 112.0(3) | F(3) | C(1) | F(1) | 106.5(5) |
| O(4) | C(9) | O(3) | 126.8(4) | F(3) | C(1) | C(2) | 111.9(4) |
| O(4) | C(9) | C(8) | 118.1(3) | F(1) | C(1) | C(2) | 111.6(5) |
| O(3) | C(9) | C(8) | 115.0(3) | F(2) | C(1) | F(3) | 106.6(5) |
| C(5) | C(4) | C(2) | 111.7(3) | F(2) | C(1) | F(1) | 106.5(4) |
| C(6) | C(7) | C(8) | 110.7(3) | F(2) | C(1) | C(2) | 113.3(4) |

TABLE 33
Hydrogen Bonds for X-Ray Crystal Structure of Compound 324

| | | | d(D − | d(H − | d(D − | D − H − |
|---|---|---|---|---|---|---|
| D | H | A | H)/Å | A)/Å | A)/Å | A/° |
| N(2) | H(2A) | O(2)1 | 0.89 | 2.10 | 2.858(4) | 142.8 |
| N(2) | H(2B) | O(4)2 | 0.89 | 1.97 | 2.812(4) | 156.3 |
| N(2) | H(2C) | O(3)3 | 0.89 | 1.78 | 2.664(4) | 174.5 |
| O(1) | H(1) | N(1)4 | 0.82 | 2.01 | 2.807(5) | 162.4 |
| N(1) | H(1A) | O(4)1 | 0.85(5) | 2.38(5) | 3.146(4) | 151(5) |

1−X, −1/2 + Y, 1 − Z;
2−1 − X, −1/2 + Y, 1 − Z;
3+X, −1 + Y, +Z;
4+X, 1 + Y, +Z

TABLE 34
Torsion Angles for X-Ray Crystal Structure of Compound 324

| A | B | C | D | Angle/° | A | B | C | D | Angle/° |
|---|---|---|---|---|---|---|---|---|---|
| O(2) | S(1) | C(6) | C(7) | 71.5(4) | C(4) | C(2) | C(1) | F(1) | −59.4(5) |
| O(2) | S(1) | C(5) | C(4) | −54.8(4) | C(4) | C(2) | C(1) | F(2) | −179.6(5) |
| N(2) | C(8) | C(9) | O(4) | −11.2(5) | C(7) | C(8) | C(9) | O(4) | 110.5(4) |
| N(2) | C(8) | C(9) | O(3) | 169.0(4) | C(7) | C(8) | C(9) | O(3) | −69.3(4) |
| N(2) | C(8) | C(7) | C(6) | −163.4(3) | C(6) | S(1) | C(5) | C(4) | −167.2(3) |
| O(1) | C(2) | C(1) | F(3) | −61.3(5) | C(5) | S(1) | C(6) | C(7) | −174.7(3) |
| O(1) | C(2) | C(1) | F(1) | 179.5(4) | C(5) | C(4) | C(2) | O(1) | −56.3(5) |
| O(1) | C(2) | C(1) | F(2) | 59.3(6) | C(5) | C(4) | C(2) | C(1) | −174.2(4) |
| N(1) | S(1) | C(6) | C(7) | −61.6(4) | C(5) | C(4) | C(2) | C(3) | 64.5(5) |
| N(1) | S(1) | C(5) | C(4) | 73.9(4) | C(2) | C(4) | C(5) | S(1) | 164.2(3) |
| C(8) | C(7) | C(6) | S(1) | −162.4(3) | C(3) | C(2) | C(1) | F(3) | −176.9(5) |
| C(9) | C(8) | C(7) | C(6) | 74.9(4) | C(3) | C(2) | C(1) | F(1) | 63.8(5) |
| C(4) | C(2) | C(1) | F(3) | 59.8(6) | C(3) | C(2) | C(1) | F(2) | −56.3(7) |

### Example 14: X-Ray Crystallography of a Hydrate of Compound 328

A 10 mg sample of compound 328 was dissolved in 200 μL H2O and then 2 mL acetonitrile was added above the H2O. The solution was kept in a sealed 4 mL vial. A crystal was observed in the fifth day. The crystal was a colourlessneedle with the following dimensions: 0.30×0.04×0.02 mm3. The symmetry of the crystal structure was assigned the orthorhombic space group P212121with the following parameters: a=5.31920 (10) Å, b=9.2091 (2) Å, c=26.9387 (6) Å, α=90°, β=90°, γ=90°, V=1319.59 (5) Å3, Z=4, Dc=1.562 g/cm3, F(000)=648.0, μ(CuKα)=2.746 mm-1, and T=293 (2) K. A total of 23177 reflections were collected in the 2θ range from 6.562 to 132.842. The limiting indices were:-6≤h≤6, −10≤k≤9, −32≤1 k≤31; which yielded 2339 unique reflections (Rint=0.0810). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 181, compared with 2339 data. All reflections were included in the refinement. The goodness of fit on F² was 1.066 with a final R value for [I>2σ (I)]R1=0.0322 and wR2=0.0873. The largest differential peak and hole were 0.24 and −0.31 Å-3, respectively. Data was collected using a Rigaku Oxford Diffraction XtaLAB Synergy-S four-circle diffractometer equipped with a HyPix-6000HE area detector. The cryogenic system used was Oxford Cryostream 800. Cu radiation was used (λ=1.54184 Å, 50 W, Micro focus source with multilayer mirror (μ-CMF)) with a d=35 mm crystal distance from the detector (Tube Voltage=50 kV; Tube Current 1 mA).

TABLE 35
Summary of crystallographic data for a hydrate of Compound 328.

| Crystal size/mm3 | 0.30 × 0.04 × 0.02 | |
|---|---|---|
| Radiation Type | CuKα | (λ = 1.54184) |
| Crystal system | orthorhombic | |
| Space group | P212121 | |
| a/Å | 5.31920 | (10) |
| b/Å | 9.2091 | (2) |
| c/Å | 26.9387 | (6) |
| α/° | 90 | |
| β/° | 90 | |
| γ/° | 90 | |
| Cell Volume/Å3 | 1319.59 | (5) |
| Cell Formula Units Z | 4 | |
| Crystal Density calc g/cm3 | 1.562 | |
| Crystal F(000) | 648.0 | |
| Absorption Coefficient μ/mm−1 | 2.746 | |
| Index ranges | −6 ≤ h ≤ 6, −10 ≤ k ≤ 9, −32 ≤ | |
| | l ≤ 31 | |
| Cell Measurement Temperature/K | 293 | (2) |
| 2θ range for data collection/° | 6.562 to 132.842 | |
| Goodness-of-fit on F2 | 1.066 | |
| Final R indexes [I >= 2σ (I)] | R1 = 0.0322, wR2 = 0.0873 | |
| Final R indexes [all data] | R1 = 0.0344, wR2 = 0.0883 | |
| Largest diff. peak/hole/e Å−3 | 0.24/−0.31 | |
| Reflections collected/unique | 23177/2339 | [Rint = 0.0810] |
| Flack parameter | −0.002 | (12) |

TABLE 36
Atomic Coordinates (×10\hat{ }4) and equivalent
isotropic displacement parameters (A\hat{ }2 ×
10\hat{ }3) for X-Ray Crystal Structure of a hydrate of Compound 328.

| Atom | | x | | y | | z | | U(eq) | |
|---|---|---|---|---|---|---|---|---|---|
| S | (1) | 7745.2 | (13) | 9516.9 | (7) | 6175.4 | (2) | 36.3 | (2) |
| O | (3) | 2738 | (4) | 11390 | (2) | 7655.9 | (8) | 41.2 | (5) |
| O | (4) | 3073 | (4) | 9116 | (2) | 7907.7 | (10) | 52.2 | (6) |
| N | (2) | 7970 | (4) | 8969 | (3) | 7855.5 | (8) | 31.3 | (5) |
| O | (5) | 2420 | (6) | 2534 | (3) | 6167.7 | (10) | 61.8 | (7) |
| O | (1) | 3842 | (4) | 5292 | (3) | 5946.2 | (12) | 60.5 | (7) |
| O | (2) | 10418 | (4) | 9252 | (3) | 6235.4 | (9) | 55.4 | (6) |
| F | (3) | 5544 | (7) | 3741 | (3) | 5128.5 | (10) | 88.0 | (9) |
| F | (1) | 5827 | (8) | 6014 | (3) | 4997.3 | (9) | 100.6 | (11) |
| C | (8) | 3949 | (5) | 10273 | (3) | 7746.3 | (10) | 29.0 | (6) |
| F | (2) | 9139 | (7) | 4803 | (4) | 5157.6 | (10) | 103.4 | (11) |
| C | (7) | 6808 | (4) | 10324 | (3) | 7660.8 | (10) | 28.8 | (5) |
| C | (4) | 6459 | (5) | 7991 | (3) | 5855.7 | (11) | 37.1 | (6) |
| C | (3) | 7722 | (6) | 6572 | (3) | 6007.4 | (11) | 40.0 | (6) |
| C | (6) | 7473 | (5) | 10564 | (3) | 7116.7 | (9) | 31.8 | (5) |
| N | (1) | 6883 | (7) | 10952 | (3) | 5950.4 | (11) | 54.3 | (8) |
| C | (5) | 6273 | (5) | 9466 | (3) | 6767.5 | (10) | 32.5 | (6) |
| C | (2) | 6395 | (6) | 5211 | (3) | 5825.0 | (12) | 40.9 | (7) |
| C | (1) | 6718 | (8) | 4951 | (4) | 5278.3 | (14) | 58.1 | (10) |

TABLE 37
Bond Lengths for X-Ray Crystal Structure
of a hydrate of Compound 328

| Atom | | Atom | | Length/Å | | Atom | | Atom | | Length/Å | |
|---|---|---|---|---|---|---|---|---|---|---|---|
| S | (1) | O | (2) | 1.452 | (2) | F | (1) | C | (1) | 1.325 | (5) |
| S | (1) | C | (4) | 1.785 | (3) | C | (8) | C | (7) | 1.539 | (3) |
| S | (1) | N | (1) | 1.524 | (3) | F | (2) | C | (1) | 1.335 | (5) |
| S | (1) | C | (5) | 1.778 | (3) | C | (7) | C | (6) | 1.524 | (4) |
| O | (3) | C | (8) | 1.238 | (3) | C | (4) | C | (3) | 1.524 | (4) |
| O | (4) | C | (8) | 1.241 | (4) | C | (3) | C | (2) | 1.520 | (4) |
| N | (2) | C | (7) | 1.489 | (4) | C | (6) | C | (5) | 1.522 | (4) |
| O | (1) | C | (2) | 1.399 | (4) | C | (2) | C | (1) | 1.502 | (5) |
| F | (3) | C | (1) | 1.340 | (5) | | | | | | |

TABLE 38
Bond Angles for X-Ray Crystal Structure
of a hydrate of Compound 328

| Atom | Atom | Atom | Angle/° | Atom | Atom | Atom | Angle/° |
|---|---|---|---|---|---|---|---|
| O(2) | S(1) | C(4) | 107.27(14) | C(2) | C(3) | C(4) | 114.5(2) |
| O(2) | S(1) | N(1) | 118.98(17) | C(5) | C(6) | C(7) | 113.6(2) |
| O(2) | S(1) | C(5) | 109.10(14) | C(6) | C(5) | S(1) | 110.62(18) |
| N(1) | S(1) | C(4) | 112.06(16) | O(1) | C(2) | C(3) | 109.4(3) |
| N(1) | S(1) | C(5) | 104.31(16) | O(1) | C(2) | C(1) | 110.4(3) |
| C(5) | S(1) | C(4) | 104.08(13) | C(1) | C(2) | C(3) | 113.3(3) |
| O(3) | C(8) | O(4) | 125.9(2) | F(3) | C(1) | C(2) | 112.0(3) |
| O(3) | C(8) | C(7) | 117.4(2) | F(1) | C(1) | F(3) | 106.0(3) |
| O(4) | C(8) | C(7) | 116.7(2) | F(1) | C(1) | F(2) | 106.3(4) |
| N(2) | C(7) | C(8) | 109.4(2) | F(1) | C(1) | C(2) | 113.7(3) |
| N(2) | C(7) | C(6) | 111.3(2) | F(2) | C(1) | F(3) | 106.9(3) |
| C(6) | C(7) | C(8) | 112.2(2) | F(2) | C(1) | C(2) | 111.4(3) |
| C(3) | C(4) | S(1) | 112.1(2) | | | | |

TABLE 39
Hydrogen Bonds for X-Ray Crystal Structure
of a hydrate of Compound 328

| | | | d(D − | d(H − | d(D − | D − H − |
|---|---|---|---|---|---|---|
| D | H | A | H)/Å | A)/Å | A)/Å | A/° |
| N(2) | H(2A) | O(3)1 | 0.89 | 1.91 | 2.771(3) | 163.7 |
| N(2) | H(2B) | O(5)2 | 0.89 | 2.15 | 2.952(3) | 149.2 |
| N(2) | H(2C) | O(4)3 | 0.89 | 1.84 | 2.721(3) | 170.8 |
| O(5) | H(5D) | N(1)4 | 0.85 | 2.03 | 2.846(5) | 160.4 |
| O(1) | H(1) | O(5) | 0.82 | 1.92 | 2.716(3) | 162.0 |

11 − X, −1/2 + Y, 3/2 − Z;
21 − X, 1/2 + Y, 3/2 − Z;
31 + X, +Y, +Z;
4+X, −1 + Y, +Z

TABLE 40
Torsion Angles for X-Ray Crystal Structure of a hydrate of Compound 328

| A | B | C | D | Angle/° | A | B | C | D | Angle/° |
|---|---|---|---|---|---|---|---|---|---|
| S(1) | C(4) | C(3) | C(2) | 168.9(2) | C(8) | C(7) | C(6) | C(5) | −54.4(3) |
| O(3) | C(8) | C(7) | N(2) | 172.1(2) | C(7) | C(6) | C(5) | S(1) | −164.94(19) |
| O(3) | C(8) | C(7) | C(6) | −63.8(3) | C(4) | S(1) | C(5) | C(6) | 171.12(19) |
| O(4) | C(8) | C(7) | N(2) | −6.9(3) | C(4) | C(3) | C(2) | O(1) | −50.3(4) |
| O(4) | C(8) | C(7) | C(6) | 117.2(3) | C(4) | C(3) | C(2) | C(1) | 73.3(4) |
| N(2) | C(7) | C(6) | C(5) | 68.6(3) | C(3) | C(2) | C(1) | F(3) | 179.6(3) |
| O(1) | C(2) | C(1) | F(3) | −57.3(4) | C(3) | C(2) | C(1) | F(1) | −60.3(4) |
| O(1) | C(2) | C(1) | F(1) | 62.8(4) | C(3) | C(2) | C(1) | F(2) | 59.8(4) |
| O(1) | C(2) | C(1) | F(2) | −177.1(3) | N(1) | S(1) | C(4) | C(3) | 168.2(2) |
| O(2) | S(1) | C(4) | C(3) | 35.9(3) | N(1) | S(1) | C(5) | C(6) | −71.3(2) |
| O(2) | S(1) | C(5) | C(6) | 56.9(2) | C(5) | S(1) | C(4) | C(3) | −79.7(2) |

### Example 15: X-Ray Crystallography of Compound 348

A 48 mg sample of compound 348 was dissolved in 2.8 mL water/methanol (2:5) and kept in a 4 mL vial. The solution evaporated slowly at room temperature. Crystals were observed in the second day. The crystal was a colourless plate with the following dimensions: 0.30×0.20×0.03 mm3. The symmetry of the crystal structure was assigned the monoclinic space group P21 with the following parameters: a=5.87510 (10) Å, b=6.71400 (10) Å, c=21.0819 (3) Å, α=90°, β=94.4900 (10)°, γ=90°, V=829.03 (2) Å3, Z=2, Dc=1.476 g/cm3, F(000)=384.0, μ(CuKα)=2.243 mm-1, and T=150.00 (10) K. A total of 20488 reflections were collected in the 2θ range from 4.204 to 133.132. The limiting indices were:-6≤h≤6, −7≤k≤7, −25≤l≤25; which yielded 2740 unique reflections (Rint=0.0819). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 242, compared with 2740 data. All reflections were included in the refinement. The goodness of fit on F² was 1.068 with a final R value for [I>2a (I)]R_{1=0.0552} and wR2=0.1502. The largest differential peak and hole were 0.78 and −0.41 Å-3. Data was collected using a Rigaku Oxford Diffraction XtaLAB Synergy-S four-circle diffractometer equipped with a HyPix-6000HE area detector. The cryogenic system used was Oxford Cryostream 800. Cu radiation was used (λ=1.54184 Å, 50 W, Micro focus source with multilayer mirror (μ-CMF)) with a d=35 mm crystal distance from the detector (Tube Voltage=50 kV; Tube Current 1 mA).

TABLE 41
Summary of crystallographic data for Compound 348.

| Crystal size/mm3 | 0.30 × 0.20 × 0.03 | |
|---|---|---|
| Radiation Type | CuKα | (λ = 1.54184) |
| Crystal system | monoclinic | |
| Space group | P21 | |
| a/Å | 5.87510 | (10) |
| b/Å | 6.71400 | (10) |
| c/Å | 21.0819 | (3) |
| α/° | 90 | |
| β/° | 94.4900 | (10) |
| γ/° | 90 | |
| Cell Volume/Å3 | 829.03 | (2) |
| Cell Formula Units Z | 2 | |
| Crystal Density calc g/cm3 | 1.476 | |
| Crystal F(000) | 384.0 | |
| Absorption Coefficient μ/mm−1 | 2.243 | |
| Index ranges | −6 ≤ h ≤ 6, −7 ≤ k ≤ 7, −25 ≤ | |
| | l ≤ 25 | |
| Cell Measurement Temperature/K | 150.00 | (10) |
| 2θ range for data collection/° | 4.204 to 133.132 | |
| Goodness-of-fit on F2 | 1.068 | |
| Final R indexes [I >= 2σ (I)] | R1 = 0.0552, wR2 = 0.1502 | |
| Final R indexes [all data] | R1 = 0.0557, wR2 = 0.1505 | |
| Largest diff. peak/hole/e Å−3 | 0.78/−0.41 | |
| Reflections collected/unique | 20488/2740 | [Rint = 0.0819] |
| Flack parameter | 0.057 | (15) |

TABLE 42
Atomic Coordinates (×10\hat{ }4) and equivalent
isotropic displacement parameters (A\hat{ }2 ×
10\hat{ }3) for X-Ray Crystal Structure of Compound 348.

| Atom | x | y | z | U(eq) |
|---|---|---|---|---|
| S(1) | 4510(2) | 5090(2) | 3444.5(6) | 19.2(3) |
| O(2) | 10932(6) | 960(7) | 5025(2) | 25.5(10) |
| O(1) | 12166(7) | −316(7) | 4132(2) | 28.3(11) |
| O(3) | 6430(7) | 6424(7) | 3348(2) | 28.1(10) |
| F(3) | −2131(7) | 9780(8) | 1418(2) | 48.8(12) |
| F(1) | 1218(9) | 10668(7) | 1794(3) | 60.7(15) |
| O(4) | −1915(7) | 6356(10) | 2173(2) | 43.2(14) |
| N(2) | 3121(9) | 5359(10) | 4017(3) | 27.4(12) |
| N(1) | 6690(8) | −640(8) | 4610(2) | 21.5(11) |
| F(2) | −1453(11) | 10308(10) | 2419(2) | 77(2) |
| C(6) | 1953(11) | 7248(10) | 2577(3) | 27.0(14) |
| C(3) | 7473(10) | 2456(10) | 4017(3) | 24.1(13) |
| C(8) | −567(13) | 9537(12) | 1913(3) | 39.8(19) |
| C(9) | 780(20) | 6360(20) | 1421(6) | 26.1(13) |
| C(10) | 2870(20) | 6710(30) | 1175(6) | 39(4) |
| C(11) | 3420(30) | 5750(40) | 622(5) | 55(5) |
| C(12) | 1870(50) | 4440(30) | 315(5) | 58(6) |
| C(13) | −220(50) | 4080(20) | 561(6) | 62(6) |
| C(14) | −760(30) | 5040(30) | 1114(7) | 52(4) |
| C(1) | 10662(8) | 310(11) | 4479(3) | 21.3(12) |
| C(5) | 2745(9) | 5145(12) | 2711(3) | 26.5(12) |
| C(7) | 77(10) | 7357(11) | 2022(3) | 26.1(13) |
| C(2) | 8192(9) | 289(11) | 4160(2) | 20.0(11) |
| C(4) | 5528(9) | 2598(9) | 3496(3) | 21.2(13) |
| C(9R) | 930(40) | 6560(40) | 1397(10) | 33(4) |
| C(10R) | 2920(40) | 7350(50) | 1186(9) | 33(4) |
| C(11R) | 3840(30) | 6560(50) | 653(10) | 51(7) |
| C(12R) | 2760(60) | 4970(40) | 330(8) | 49(7) |
| C(13R) | 770(60) | 4180(30) | 540(9) | 45(8) |
| C(14R) | −150(50) | 4970(40) | 1074(11) | 40(6) |

TABLE 43
Bond Lengths for X-Ray Crystal Structure of Compound 348

| Atom | | Atom | | Length/Å | | Atom | | Atom | | Length/Å | |
|---|---|---|---|---|---|---|---|---|---|---|---|
| S | (1) | O | (3) | 1.467 | (4) | C | (9) | C | (10) | 1.3900 | |
| S | (1) | N | (2) | 1.520 | (5) | C | (9) | C | (14) | 1.3900 | |
| S | (1) | C | (5) | 1.794 | (5) | C | (9) | C | (7) | 1.521 | (10) |
| S | (1) | C | (4) | 1.777 | (6) | C | (10) | C | (11) | 1.3900 | |
| O | (2) | C | (1) | 1.231 | (7) | C | (11) | C | (12) | 1.3900 | |
| O | (1) | C | (1) | 1.263 | (7) | C | (12) | C | (13) | 1.3900 | |
| F | (3) | C | (8) | 1.345 | (8) | C | (13) | C | (14) | 1.3900 | |
| F | (1) | C | (8) | 1.334 | (9) | C | (1) | C | (2) | 1.551 | (7) |
| O | (4) | C | (7) | 1.408 | (8) | C | (7) | C | (9R) | 1.542 | (13) |
| N | (1) | C | (2) | 1.483 | (7) | C | (9R) | C | (10R) | 1.3900 | |
| F | (2) | C | (8) | 1.328 | (9) | C | (9R) | C | (14R) | 1.3900 | |
| C | (6) | C | (5) | 1.506 | (10) | C | (10R) | C | (11R) | 1.3900 | |
| C | (6) | C | (7) | 1.545 | (8) | C | (11R) | C | (12R) | 1.3900 | |
| C | (3) | C | (2) | 1.538 | (9) | C | (12R) | C | (13R) | 1.3900 | |
| C | (3) | C | (4) | 1.525 | (7) | C | (13R) | C | (14R) | 1.3900 | |
| C | (8) | C | (7) | 1.525 | (10) | | | | | | |

TABLE 44
Bond Angles for X-Ray Crystal Structure of Compound 348

| Atom | Atom | Atom | Angle/° | Atom | Atom | Atom | Angle/° |
|---|---|---|---|---|---|---|---|
| O(3) | S(1) | N(2) | 120.3(3) | O(1) | C(1) | C(2) | 114.8(5) |
| O(3) | S(1) | C(5) | 105.4(3) | C(6) | C(5) | S(1) | 109.2(5) |
| O(3) | S(1) | C(4) | 108.9(3) | O(4) | C(7) | C(6) | 111.2(5) |
| N(2) | S(1) | C(5) | 111.9(3) | O(4) | C(7) | C(8) | 106.9(5) |
| N(2) | S(1) | C(4) | 105.2(3) | O(4) | C(7) | C(9) | 105.3(7) |
| C(4) | S(1) | C(5) | 103.9(3) | O(4) | C(7) | C(9R) | 111.0(10) |
| C(5) | C(6) | C(7) | 112.2(5) | C(8) | C(7) | C(6) | 108.3(5) |
| C(4) | C(3) | C(2) | 112.3(5) | C(8) | C(7) | C(9R) | 107.3(13) |
| F(3) | C(8) | C(7) | 112.3(6) | C(9) | C(7) | C(6) | 112.6(7) |
| F(1) | C(8) | F(3) | 106.6(6) | C(9) | C(7) | C(8) | 112.3(9) |
| F(1) | C(8) | C(7) | 112.7(6) | C(9R) | C(7) | C(6) | 111.8(11) |
| F(2) | C(8) | F(3) | 106.8(6) | N(1) | C(2) | C(3) | 110.8(5) |
| F(2) | C(8) | F(1) | 107.0(7) | N(1) | C(2) | C(1) | 108.1(4) |
| F(2) | C(8) | C(7) | 111.2(6) | C(3) | C(2) | C(1) | 108.0(5) |
| C(10) | C(9) | C(14) | 120.0 | C(3) | C(4) | S(1) | 109.4(4) |
| C(10) | C(9) | C(7) | 122.8(8) | C(10R) | C(9R) | C(7) | 118.5(13) |
| C(14) | C(9) | C(7) | 117.2(8) | C(10R) | C(9R) | C(14R) | 120.0 |
| C(11) | C(10) | C(9) | 120.0 | C(14R) | C(9R) | C(7) | 121.3(13) |
| C(10) | C(11) | C(12) | 120.0 | C(11R) | C(10R) | C(9R) | 120.0 |
| C(13) | C(12) | C(11) | 120.0 | C(10R) | C(11R) | C(12R) | 120.0 |
| C(12) | C(13) | C(14) | 120.0 | C(13R) | C(12R) | C(11R) | 120.0 |
| C(13) | C(14) | C(9) | 120.0 | C(12R) | C(13R) | C(14R) | 120.0 |
| O(2) | C(1) | O(1) | 128.0(5) | C(13R) | C(14R) | C(9R) | 120.0 |
| O(2) | C(1) | C(2) | 117.2(5) | | | | |

TABLE 45
Hydrogen Bonds for X-Ray Crystal Structure of Compound 348

| | | | d(D − | d(H − | d(D − | D − H − |
|---|---|---|---|---|---|---|
| D | H | A | H)/Å | A)/Å | A)/Å | A/° |
| O(4) | H(4) | O(3)1 | 0.84 | 1.93 | 2.730(7) | 158.8 |
| N(1) | H(1A) | O(1)1 | 0.91 | 1.87 | 2.776(6) | 179.3 |
| N(1) | H(1B) | N(2)2 | 0.91 | 2.10 | 2.965(7) | 157.9 |
| N(1) | H(1C) | O(2)3 | 0.91 | 1.92 | 2.755(7) | 151.4 |
| N(2) | H(2) | O(1)4 | 0.74(10) | 2.25(11) | 2.971(8) | 164(9) |

1−1 + X, +Y, +Z;
21 − X, −1/2 + Y, 1 − Z;
32 − X, −1/2 + Y, 1 − Z;
4−1 + X, 1 + Y, +Z

TABLE 46
Torsion Angles for X-Ray Crystal Structure of Compound 348

| A | B | C | D | Angle/° | A | B | C | D | Angle/° |
|---|---|---|---|---|---|---|---|---|---|
| O(2) | C(1) | C(2) | N(1) | −49.2(8) | C(10) | C(9) | C(7) | C(6) | −48.4(14) |
| O(2) | C(1) | C(2) | C(3) | 70.6(7) | C(10) | C(9) | C(7) | C(8) | 74.2(13) |
| O(1) | C(1) | C(2) | N(1) | 132.2(6) | C(10) | C(11) | C(12) | C(13) | 0.0 |
| O(1) | C(1) | C(2) | C(3) | −108.0(6) | C(11) | C(12) | C(13) | C(14) | 0.0 |
| O(3) | S(1) | C(5) | C(6) | −59.9(5) | C(12) | C(13) | C(14) | C(9) | 0.0 |
| O(3) | S(1) | C(4) | C(3) | 55.2(5) | C(14) | C(9) | C(10) | C(11) | 0.0 |
| F(3) | C(8) | C(7) | O(4) | −62.6(7) | C(14) | C(9) | C(7) | O(4) | 10.4(11) |
| F(3) | C(8) | C(7) | C(6) | 177.4(6) | C(14) | C(9) | C(7) | C(6) | 131.8(9) |
| F(3) | C(8) | C(7) | C(9) | 52.4(9) | C(14) | C(9) | C(7) | C(8) | −105.6(10) |
| F(3) | C(8) | C(7) | C(9R) | 56.6(11) | C(5) | S(1) | C(4) | C(3) | 167.2(4) |
| F(1) | C(8) | C(7) | O(4) | 177.0(5) | C(5) | C(6) | C(7) | O(4) | 64.2(7) |

As one of skill in the art will readily appreciate, this disclosure has been presented for purposes of illustration and description. The disclosure above is not intended to limit the invention to the form or forms disclosed herein. Although the description of the disclosure has included description of one or more embodiments and certain variations and modifications, other variations and modifications are within the scope of the present disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A compound of Formula I: 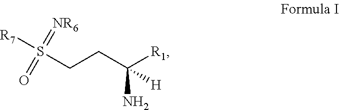
a diastereomer or enantiomer thereof, or a pharmaceutically acceptable salt of any of the foregoing, or a deuterated derivative of any of the forgoing,
wherein:
R₁ is:
C(O)OH; or
C(O)OX, wherein X is an organic cation, an inorganic cation, Na⁺, K⁺, Mg²⁺, Ca²⁺, Zn²⁺, or Mn²⁺; or 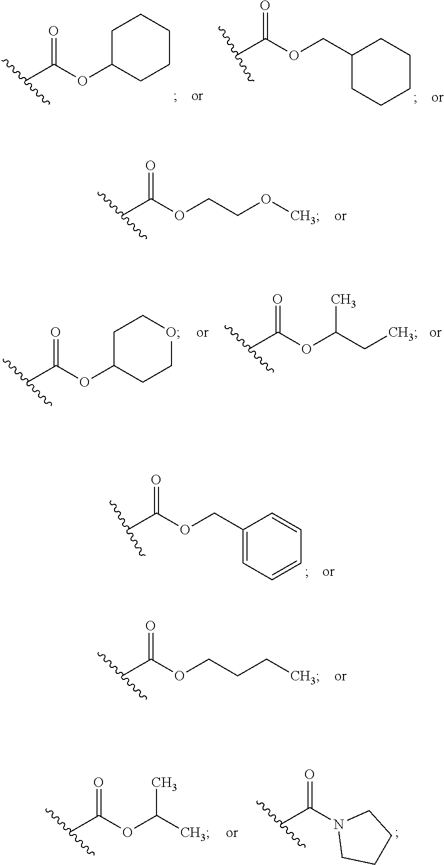
or
C(O)OR₃, wherein R₃ is a linear or branched alkyl, a cycloaklyl, a cyclic ether, or a linear or branched alkyl ether, wherein any of these is optionally and independently substituted;
or
R₃ is 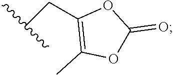 [SMILES: *Cc1oc(=O)oc1C]
or
R₃ is 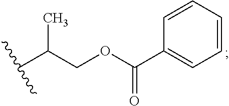 [SMILES: *C(C)COC(=O)c1ccccc1]
or
C(O)N(R₄R₅)
wherein R₄ is H; or
R₄ is a linear or branched chain alkyl, a C₁-C₁₀ linear or branched chain alkynyl, CH₂—CCH alkyne, cycloalkyl, heterocycloalkyl, aryl, or heteroaryl; wherein any of these is optionally and independently substituted; and
R₅ is absent; or
R₅ is H; or
R₅ is a linear or branched chain alkyl, cycloalkyl, heterocycloalkyl, aryl, or heteroaryl; wherein any of these is optionally and independently substituted; or
R₅ is S(O)₂alkyl; or
R₅ is S(O)₂CF₃; or
R₅ is S(O)₂NH₂; or
R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
R₅ is 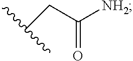 [SMILES: *CC(N)=O]
or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is 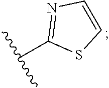 [SMILES: *c1nccs1]
or
R₅ is alkylaryl or benzyl; or
R₅ is 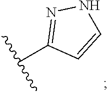 [SMILES: *c1cc[nH]n1]
or
R₅ is 2-pyridyl, 3-pyridyl; or 4-pyridyl; or
R₅ is 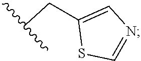 [SMILES: *Cc1cncs1]
or
R₅ is 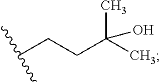 [SMILES: *CCC(C)(C)O]
or
R₅ is 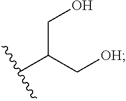 [SMILES: *C(CO)CO]
or
R₅ is 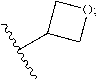 [SMILES: *C1COC1]
or
R₅ is 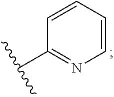 [SMILES: *c1ccccn1]
or
CN; or 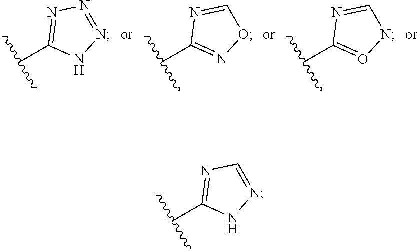
and
R₂ is: NH₂, NHC(O)OMe, or NHMe; and
R₆ is: H, C(O)Me, or P(O)(OH)₂; and
R₇ is: C₁-C₃ or C₅-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl, any of the foregoing can be independently and optionally substituted, wherein when the linear C₃-alkyl is substituted on a terminal carbon atom by a methyl group, the linear C₃-alkyl substituted on a terminal carbon atom by the methyl group contains a further substitution; wherein when the C₂-alkyl is substituted on a terminal carbon atom by an ethyl group, the C₂-alkyl substituted on a terminal carbon atom by an ethyl group contains a further substation; wherein when the C₁-alkyl is substituted on a terminal carbon atom by an n-propyl group, the C₁-alkyl substituted on a terminal carbon atom by an n-propyl group contains a further substation; or
R₇ is:

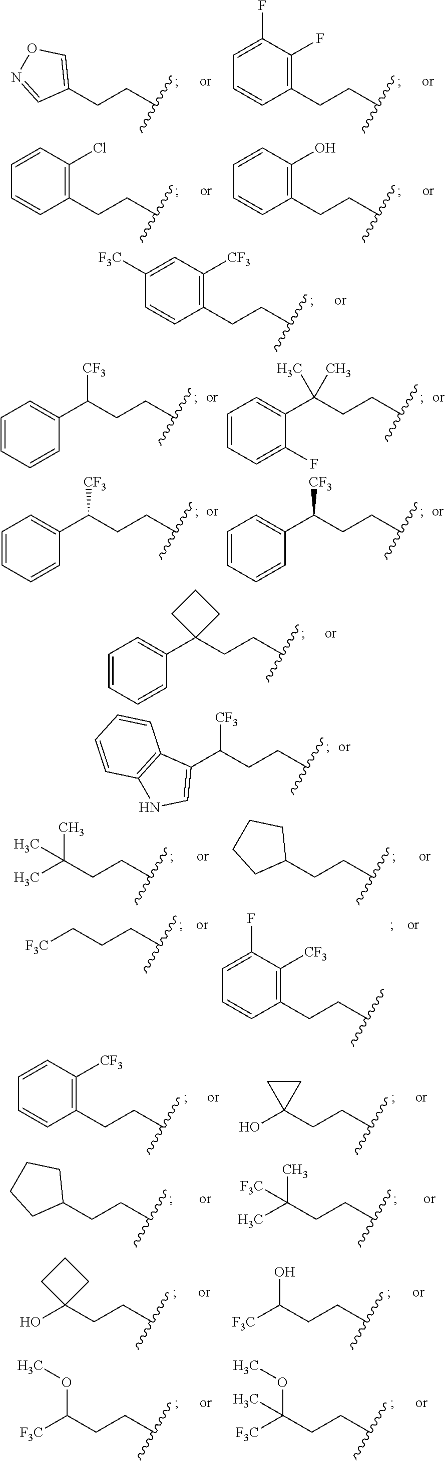

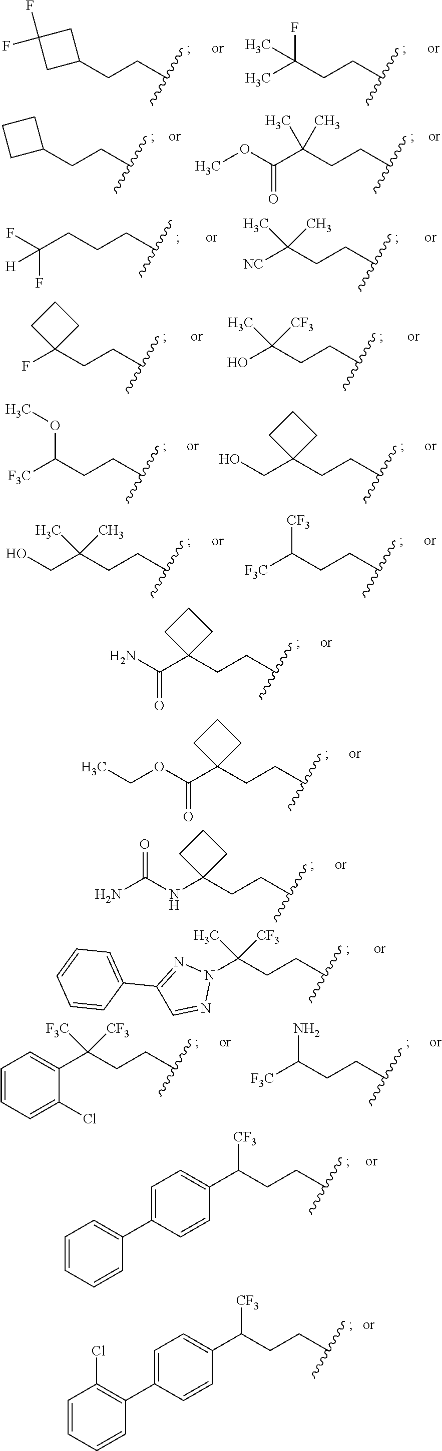

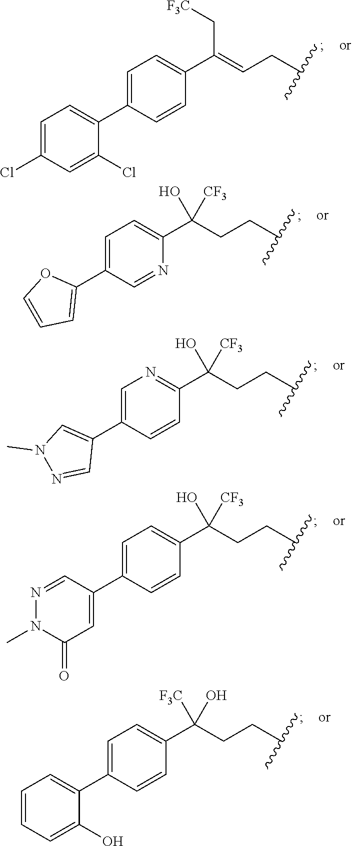

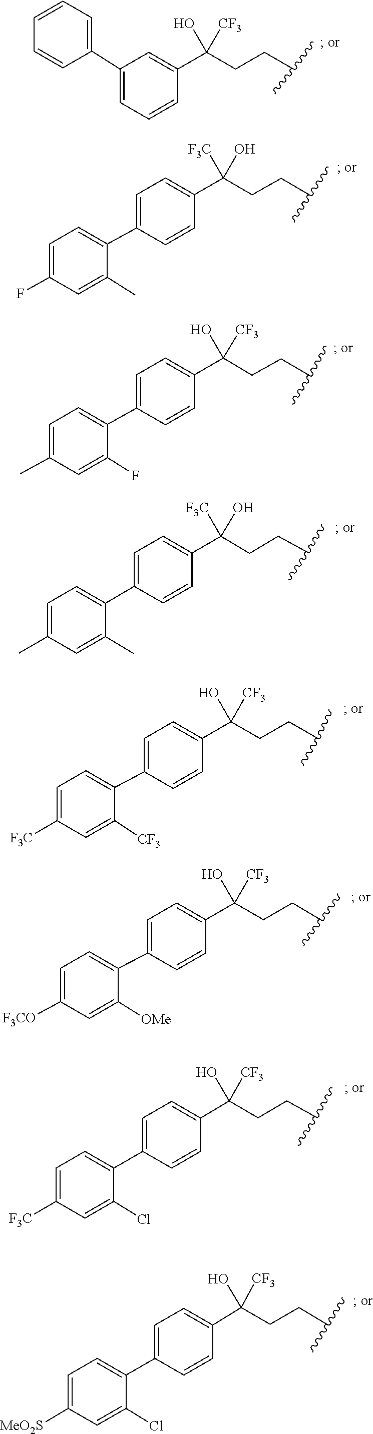

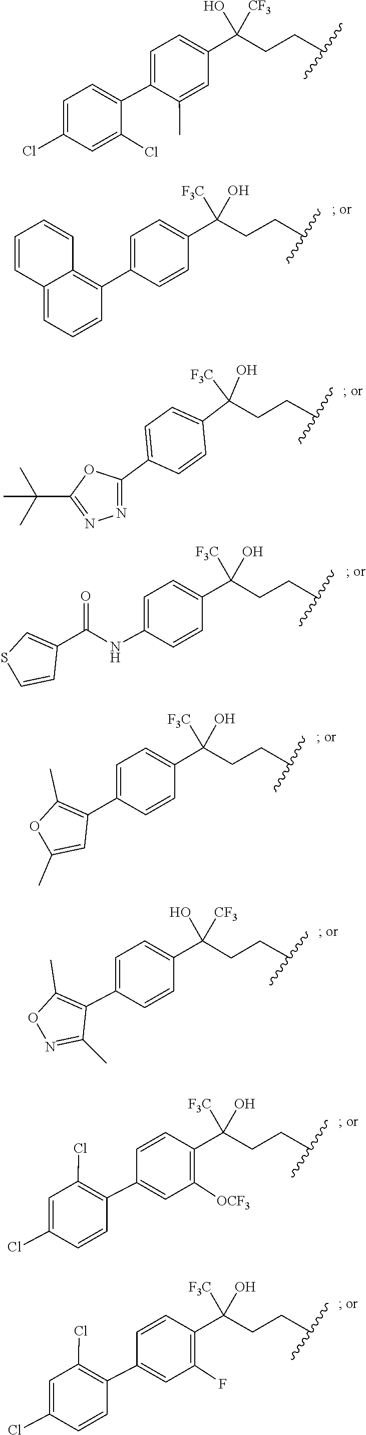

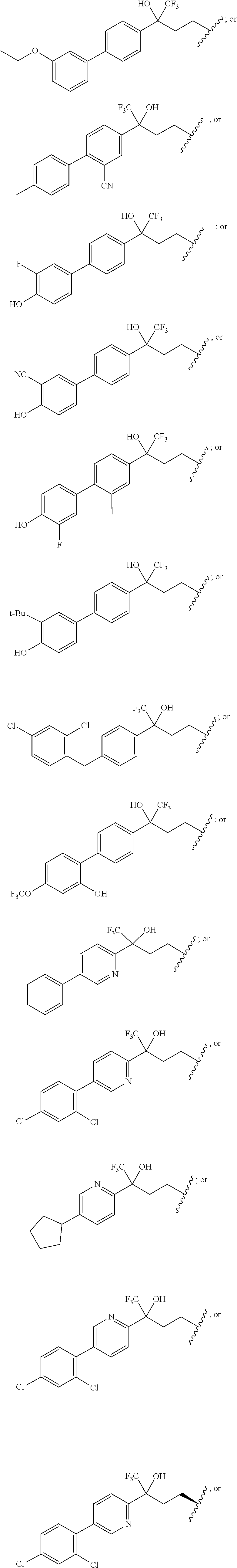

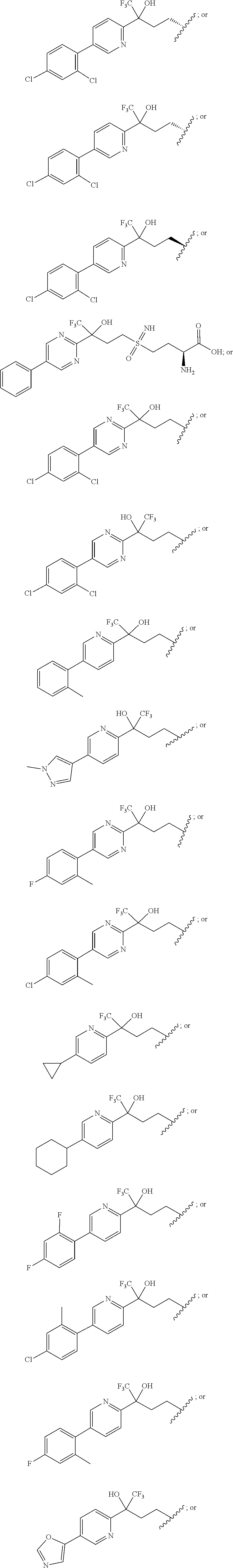

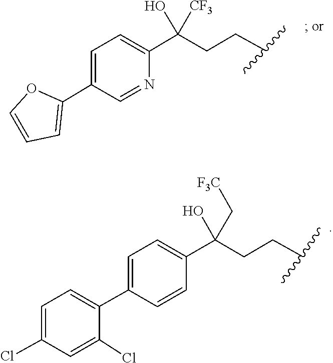 [SMILES: *CCC(O)(c1ccc(-c2ccco2)cn1)C(F)(F)F ; *CCC(O)(CC(F)(F)F)c1ccc(-c2ccc(Cl)cc2Cl)cc1]

2. The compound of Formula I of claim 1, the diastereomer or enantiomer thereof, the pharmaceutically acceptable salt of any of the foregoing, or the deuterated derivative of any of the foregoing, wherein
R₁ is: 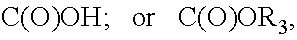
wherein R₃ is a linear or branched chain alkyl, C₁-C₁₀ linear or branched chain alkyl, methyl, ethyl, n-propyl, iso-propyl, iso-butyl, sec-butyl, n-butyl, tert-butyl, pentyl, hexyl, heptyl, octyl, nonyl, cycloalkyl, C₁-C₁₀ cycloalkyl, cyclopropyl, cyclobutyl, cyclopentyl, cyclohexyl, cycloheptyl, alkylcycloalkyl, alkylcyclohexyl, methylcyclopropyl, methylcycobutyl, methylcyclopentyl, methylcyclohexyl, methylcycloheptyl, an alkyl ether, 2-methoxyethyl, 3-methoxypropyl, 4-methoxybutyl, 5-methoxypropyl, cyclicalkylether, tetrahydropyranyl, 2-tetrahydropyranyl, 3-tetrahydropyranyl, 4-tetrahydropyranyl, tetrahydrofuranyl, 2-tetrahydrofuranyl, 2-tetrahydrofuranyl, alkylaryl, benzyl;
wherein any of these is optionally and independently substituted with one or more C₁-C₁₀ alkyl, one or more halogens, one or more fluoro, one or more chloro, one or more deuterium, bromo, one or more iodo, aryl, C₆ aryl, C₁₀ aryl, heteroaryl, a C₁-C₇ alkylcycloaklyl, an unsubstituted tetrahydropyranyl, 2-tetrahydropyranyl, 3-tetrahydropyranyl, or 4-tetrahydropyranyl or any combination thereof.

3. The compound of Formula I of claim 1, the diastereomer or enantiomer thereof, the pharmaceutically acceptable salt of any of the foregoing, or the deuterated derivative of any of the foregoing, wherein
R₁ is C(O)N(R₄R₅) and
wherein R₅ is:
C₁-C₁₀ a linear or branched chain alkyl, methyl, ethyl, propyl, or butyl; any of which are optionally or independently substituted by one or more deuterium, C₁-C₁₀ linear or branched chain alkyl, one or more halo, one or more fluoro, one or more chloro, one or more iodo, one or more or any combination thereof.

4. The compound of Formula I of claim 1, the diastereomer or enantiomer thereof, the pharmaceutically acceptable salt of any of the foregoing, or the deuterated derivative of any of the foregoing, wherein
R₁ is C(O)N(R₄R₅) and
wherein R₅ is: heteroaryl, 2-pyridyl, 3-pyridyl, or 4-pyridyl; or
R₅ is S(O)₂Me.

5. The compound of Formula I of claim 1, the diastereomer or enantiomer thereof, the pharmaceutically acceptable salt of any of the foregoing, or the deuterated derivative of any of the foregoing, wherein
R₇ is: C₁-C₃ or C₅-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₃ or C₅-C₁₀ linear or branched chain alkyl, the C₁-C₁₀ linear or branched chain alkenyl, or the C₁-C₁₀ linear or branched chain alkynyl; any of foregoing can be independently and optionally substituted by one or more of a substituent that can be:
deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an amino, a carboxylic acid or pharmaceutically acceptable salt thereof, an amide, a carbamate, a urea, an ester, an alkoxy, a methoxy, an ethoxy, a trifluoro methoxy, an ether, a cyclic ether, an C₁-C₇ alkyl ether, a C₁-C₇ cyclic alkyl ether, a trihalomethyl, a trifluoromethyl, an aryl, a heteroaryl, a fused aryl, a bi-aryl, a fused aryl-heteroaryl, a fused di-aryl, a fused aryl-heteroaryl, a 5-membered heteroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal; a ketal; or any combination of these;
wherein when the linear C₃-alkyl is substituted on a terminal carbon atom by a methyl group substituent, the linear C₃-alkyl substituted on a terminal carbon atom by the methyl group substituent contains a further substitution; wherein when the C₂-alkyl is substituted on a terminal carbon atom by an ethyl group, the C₂-alkyl substituted on a terminal carbon atom by an ethyl group contains a further substation; wherein when the C₁-alkyl is substituted on a terminal carbon atom by an n-propyl group, the C₁-alkyl substituted on a terminal carbon atom by an n-propyl group contains a further substation; and
wherein the C₁-C₁₀ linear alkyl substituent, the methyl substituent, the ethyl substituent, the C₁-C₁₀ branched chain alkyl substituent, the hydroxyl substituent, the amino substituent, the carboxylic acid or pharmaceutically acceptable salt thereof substituent, the amide substituent, the carbamate substituent, the urea substituent, the ester substituent, the alkoxy substituent, the methoxy substituent, the ethoxy substituent, the ether substituent, the cyclic ether substituent, the C₁-C₇ alkyl ether substituent, the C₁-C₇ cyclic ether substituent, the aryl substituent, the heteroaryl substituent, the fused aryl substituent, the bi-aryl substituent, the fused aryl-heteroaryl substituent, the fused di-aryl substituent, the fused aryl-heteroaryl substituent, the 5-membered heteroaryl substituent, the 6-membered heteroaryl substituent, the naphthyl substituent, the cycloalkyl substituent, the cyclopropyl substituent, the cyclobutyl substituent, the cyclopentyl substituent, the cyclohexyl substituent, the cycloheptyl substituent, the tert-butyl substituent, the bicyclic aliphatic substituent, the tricyclic aliphatic substituent, the adamantly substituent, or any combination of these can be independently and optionally substituted by one or more of:
a C₁-C₁₀ linear chain alkyl, a methyl, an ethyl, a C₁-C₁₀ branched chain alkyl, a halogen, a fluoro, a chloro, a bromo, an iodo, a hydroxyl, an alkoxy, a methoxy, an ethoxy, a carbamate, a urea, an amide, an ester, an amine, a trifluoro methoxy, an ether, an C₁-C₇ alkyl ether, a C₁-C₇ cyclic ether, a trihalomethyl, a trifluoromethyl, an aryl, a heteroaryl, a 5-membered hereroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal, a ketal, or any combination of these.

6. A compound of Formula I 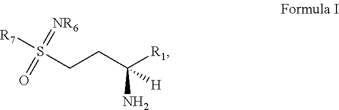
a diastereomer or enantiomer thereof, or a pharmaceutically acceptable salt of any of the foregoing, or a deuterated derivative of any of the forgoing,
wherein:
R₁ is:
C(O)OH; or
C(O)OX, wherein X is an organic cation, an inorganic cation, Na⁺, K⁺, Mg²⁺, Ca²⁺, Zn²⁺, or Mn²⁺; or 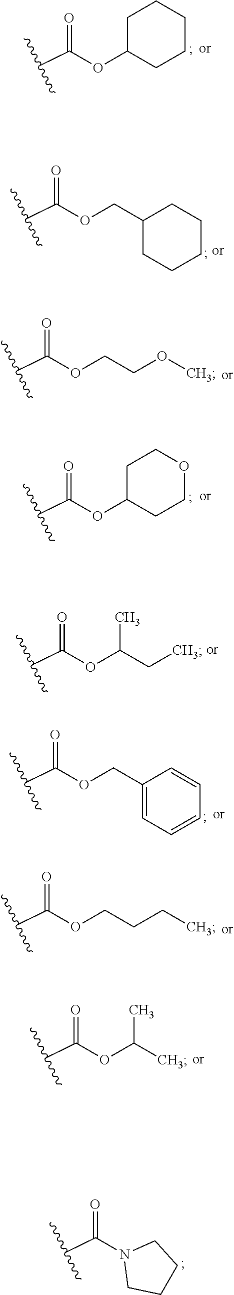
or
C(O)OR₃, wherein R₃ is a linear or branched alkyl, a cycloaklyl, a cyclic ether, or a linear or branched alkyl ether, wherein any of these is optionally and independently substituted;
or
R₃ is 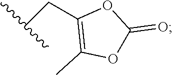 [SMILES: *Cc1oc(=O)oc1C]
or
R₃ is 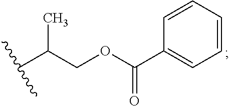 [SMILES: *C(C)COC(=O)c1ccccc1]
or
C(O)N(R₄R₅)
wherein R₄ is H; or
R₄ is a linear or branched chain alkyl, cycloalkyl, heterocycloalkyl, aryl, or heteroaryl; wherein any of these is optionally and independently substituted; and
R₅ is H; or
R₅ is a linear or branched chain alkyl, cycloalkyl, heterocycloalkyl, aryl, or heteroaryl; wherein any of these is optionally and independently substituted; or
R₅ is S(O)₂alkyl; or
R₅ is S(O)₂CF₃; or
R₅ is S(O)₂NH₂; or
R₅ is S(O)₂cycloalkyl, S(O)₂cyclopropyl, S(O)₂cyclobutyl, S(O)₂cyclopentyl, S(O)₂cyclohexyl, or S(O)₂cycloheptyl; or
R₅ is 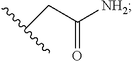 [SMILES: *CC(N)=O]
or
R₅ is cyclopropyl, cyclobutyl, cyclopentyl, cyclohexyl, or cycloheptyl; or
R₅ is pyrrolidinyl; or
R₅ is 2-tetrahydropyranyl, 3-tetrahydropyranyl; or 4-tetrahydropyranyl; or
R₅ is 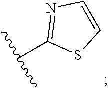 [SMILES: *c1nccs1]
or
R₅ is alkylaryl or benzyl; or
R₅ is 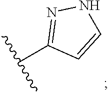 [SMILES: *c1cc[nH]n1]
or
R₅ is 2-pyridyl, 3-pyridyl or 4-pyridyl; or
R₅ is 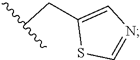 [SMILES: *Cc1cncs1]
or
R₅ is 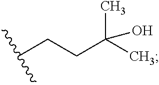 [SMILES: *CCC(C)(C)O]
or
R₅ is 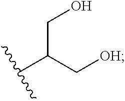 [SMILES: *C(CO)CO]
or
R₅ is 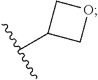 [SMILES: *C1COC1]
or
R₅ is 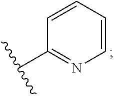 [SMILES: *c1ccccn1]
or
CN; or 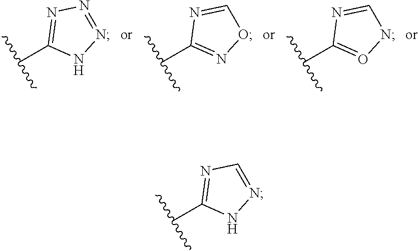
and
R₂ is: NH₂, NHC(O)OMe, or NHMe; and
R₆ is: H, C(O)Me, or P(O)(OH)₂; and
R₇ is: linear or branched chain: alkyl, alkenyl, or alkynyl, any of which can optionally and independently be substituted; or
R₇ is: 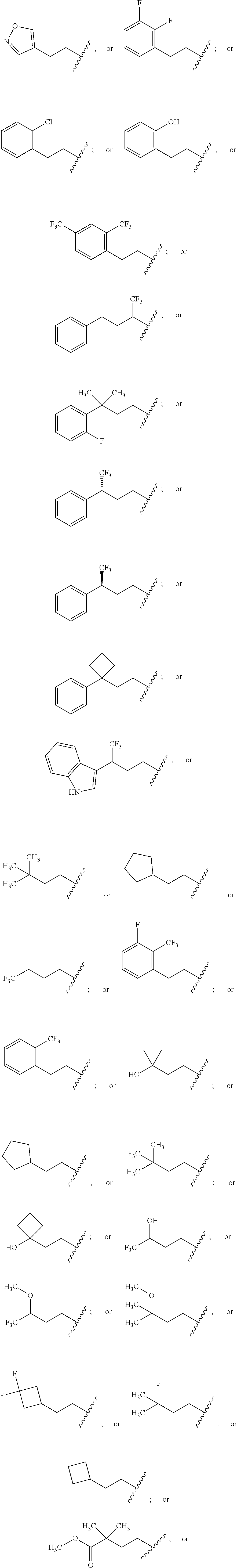 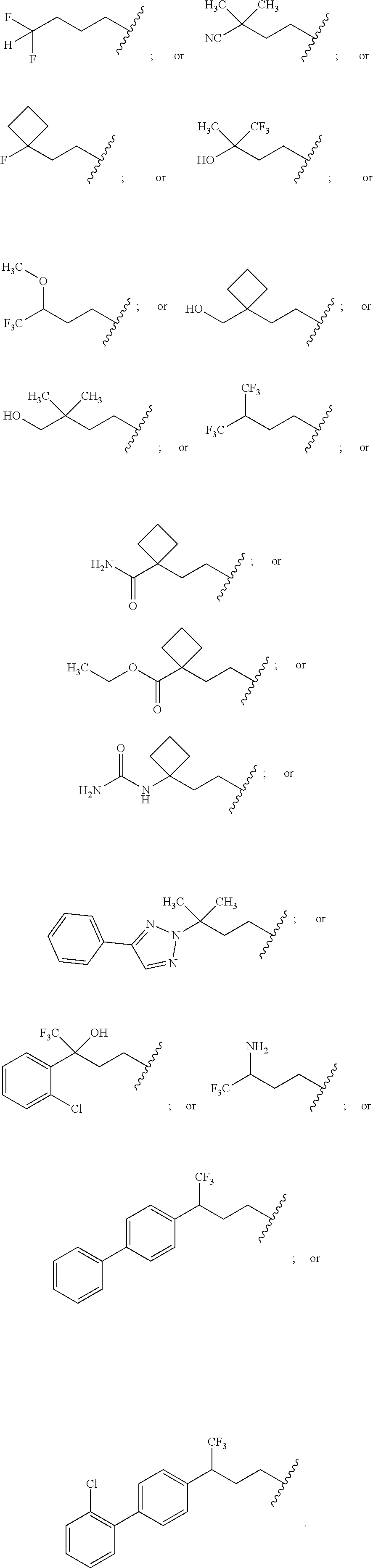
and wherein the compound of Formula I is not buthionine sulfoximine (BSO) or a salt of BSO.

7. The compound of Formula I of claim 6, the diastereomer or enantiomer thereof, the pharmaceutically acceptable salt of any of the foregoing, or the deuterated derivative of any of the foregoing, wherein
R₁ is:
C(O)OH; or
C(O)OR₃, wherein R₃ is alkyl, C₁-C₁₀ linear or branched chain alkyl, methyl, ethyl, n-propyl, iso-propyl, iso-butyl, sec-butyl, n-butyl, tert-butyl, pentyl, hexyl, heptyl, octyl, nonyl, cycloalkyl, C₁-C₁₀ cycloalkyl, cyclopropyl, cyclobutyl, cyclopentyl, cyclohexyl, cycloheptyl, alkylcyclohexyl, methylcyclopropyl, methylcycobutyl, methylcyclopentyl, methylcyclohexyl, methylcycloheptyl, an alkyl ether, 2-methoxyethyl, 3-methoxypropyl, 4-methoxybutyl, 5-methoxypropyl, cyclicalkylether, tetrahydropyranyl, 2-tetrahydropyranyl, 3-tetrahydropyranyl, 4-tetrahydropyranyl, tetrahydrofuranyl, 2-tetrahydrofuranyl, 2-tetrahydrofuranyl, alkylaryl, benzyl; wherein any of these is optionally and independently substituted with one or more deuterium, C₁-C₁₀ linear or branched chain alkyl, one or more halogens, one or more fluoro, one or more chloro, one or more bromo, one or more iodo, aryl, C₆ aryl, C₁₀ aryl, heteroaryl, a C₁-C₇ alkylcycloaklyl, an unsubstituted tetrahydropyranyl, 2-tetrahydropyranyl, 3-tetrahydropyranyl, or 4-tetrahydropyranyl or any combination thereof.

8. The compound of Formula I of claim 6, the diastereomer or enantiomer thereof, the pharmaceutically acceptable salt of any of the foregoing, or the deuterated derivative of any of the foregoing, wherein
R₁ is C(O)N(R₄R₅) and
wherein R₅ is:
C₁-C₁₀ linear or branched chain alkyl, methyl, ethyl, propyl, or butyl; any of which are optionally or independently substituted by one or more deuterium, C₁-C₁₀ linear or branched chain alkyl, one or more halo, one or more fluoro, one or more chloro, one or more iodo, one or more or any combination thereof.

9. The compound of Formula I of claim 6, the diastereomer or enantiomer thereof, the pharmaceutically acceptable salt of any of the foregoing, or the deuterated derivative of any of the foregoing, wherein
R₁ is C(O)N(R₄R₅) and
wherein R₅ is: heteroaryl, 2-pyridyl, 3-pyridyl, or 4-pyridyl; or
R₅ is S(O)₂Me.

10. The compound of Formula I of claim 6, the diastereomer or enantiomer thereof, the pharmaceutically acceptable salt of any of the foregoing, or the deuterated derivative of any of the foregoing, wherein
R₇ is: C₁-C₁₀ linear or branched chain alkyl, C₁-C₁₀ linear or branched chain alkenyl, or C₁-C₁₀ linear or branched chain alkynyl, any of which can bear an alkyl ring or an alkyl ether ring which comprises one carbon of the C₁-C₁₀ linear or branched chain alkyl, the C₁-C₁₀ linear or branched chain alkenyl, or the C₁-C₁₀ linear or branched chain alkynyl; any of the foregoing can be independently substituted by one or more of a substituent that can be:
deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an amino, a carboxylic acid or pharmaceutically acceptable salt thereof, an amide, a carbamate, a urea, an ester, an alkoxy, a methoxy, an ethoxy, a trifluoro methoxy, an ether, a cyclic ether, an C₁-C₇ linear or branched alkyl ether, a C₁-C₇ cyclic alkyl ether, a trihalomethyl, a trifluoromethyl, an aryl, a heteroaryl, a fused aryl, a bi-aryl, a fused aryl-heteroaryl, a fused di-aryl, a fused aryl-heteroaryl, a 5-membered heteroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal; a ketal; or any combination of these;
wherein the C₁-C₁₀ linear alkyl substituent, the methyl substituent, the ethyl substituent, the C₁-C₁₀ branched chain alkyl substituent, the hydroxyl substituent, the amino substituent, the carboxylic acid or pharmaceutically acceptable salt thereof substituent, the amide substituent, the carbamate substituent, the urea substituent, the ester substituent, the alkoxy substituent, the methoxy substituent, the ethoxy substituent, the ether substituent, the cyclic ether substituent, the C₁-C₇ linear or branched alkyl ether substituent, the C₁-C₇ cyclic ether substituent, the aryl substituent, the heteroaryl substituent, the fused aryl substituent, the bi-aryl substituent, the fused aryl-heteroaryl substituent, the fused di-aryl substituent, the fused aryl-heteroaryl substituent, the 5-membered heteroaryl substituent, the 6-membered heteroaryl substituent, the naphthyl substituent, the cycloalkyl substituent, the cyclopropyl substituent, the cyclobutyl substituent, the cyclopentyl substituent, the cyclohexyl substituent, the cycloheptyl substituent, the tert-butyl substituent, the bicyclic aliphatic substituent, the tricyclic aliphatic substituent, the adamantly substituent, or any combination of these can be independently and optionally substituted by one or more of:
deuterium, a C₁-C₁₀ linear chain alkyl, a methyl, an ethyl, a C₁-C₁₀ branched chain alkyl, a halogen, a fluoro, a chloro, a bromo, an iodo, a hydroxyl, an alkoxy, a methoxy, an ethoxy, a carbamate, a urea, an amide, an ester, an amine, a trifluoro methoxy, an ether, a C₁-C₇ linear or branched alkyl ether, a C₁-C₇ cyclic ether, a trihalomethyl, a trifluoromethyl, an aryl, a heteroaryl, a 5-membered hereroaryl, a 6-membered heteroaryl, a naphthyl, a cycloalkyl, a cyclopropyl, a cyclobutyl, a cyclopentyl, a cyclohexyl, a cycloheptyl, a tert-butyl, a bicyclic aliphatic, a tricyclic aliphatic, an adamantyl, a cyano, an acetal, a ketal, or any combination of these.

11. The compound of Formula I of claim 1 or claim 6, the diastereomer or the enantiomer thereof, the pharmaceutically acceptable salt of any of the foregoing, or the deuterated derivative of any of the foregoing, wherein R₁ is

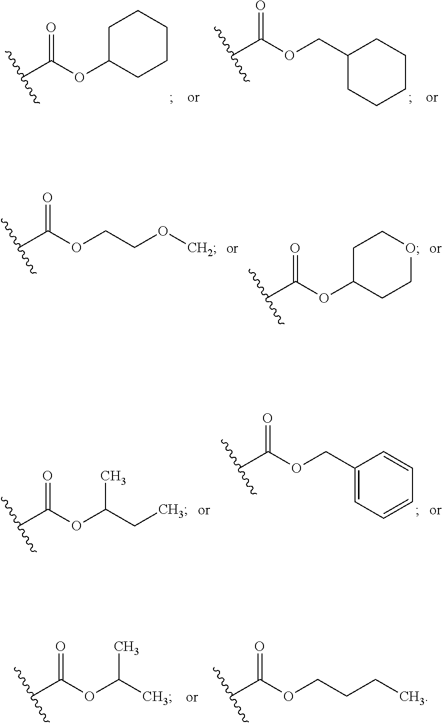

12. The compound of Formula I of claim 1 or claim 6, the diastereomer or the enantiomer thereof, the pharmaceutically acceptable salt of any of the foregoing, or the deuterated derivative of any of the foregoing; wherein R₁ is

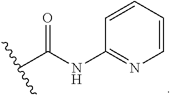 [SMILES: *C(=O)Nc1ccccn1]

13. The compound of Formula I claim 1 or claim 6, the diastereomer or the enantiomer of the foregoing, or the pharmaceutically acceptable salt of any of the foregoing, or the deuterated derivative of any of the foregoing; wherein R₂ is NH₂.

14. The compound of Formula I of claim 1 or claim 6, the diastereomer or the enantiomer of the foregoing, or the pharmaceutically acceptable salt of any of the foregoing; or the deuterated derivative of any of the foregoing; wherein R₆ is H.

15. The compound of Formula I of claim 1 or claim 6, the diastereomer or the enantiomer of the foregoing, or the pharmaceutically acceptable salt any of the foregoing, or the deuterated derivative of any of the foregoing; wherein R₇ is

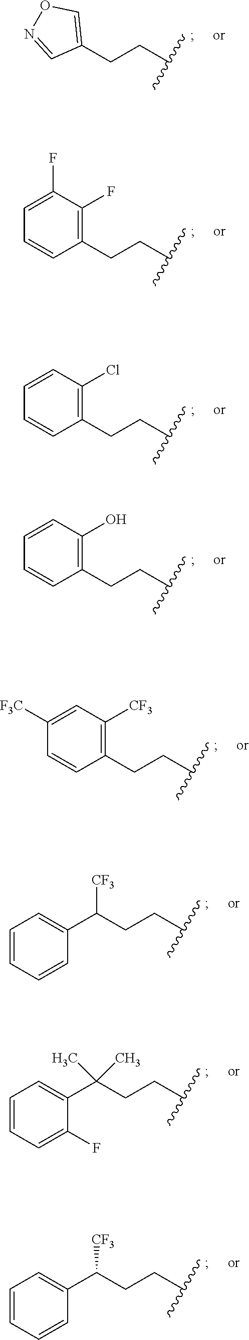

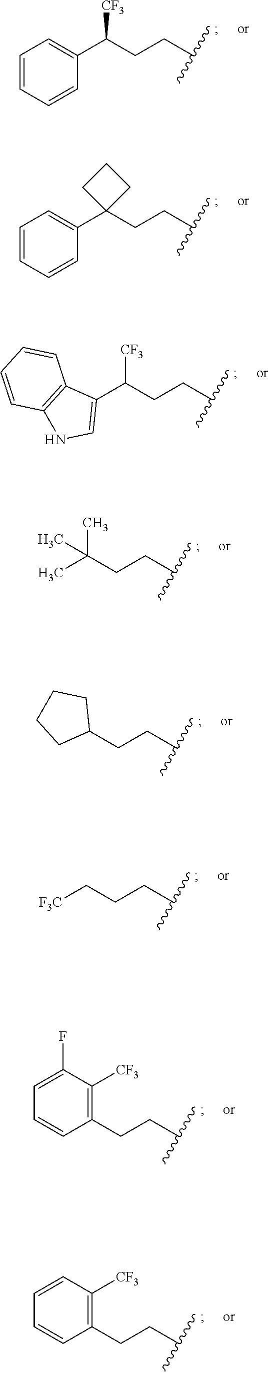

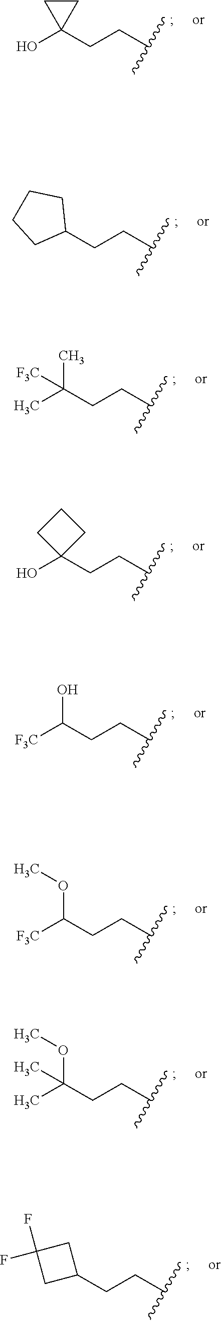

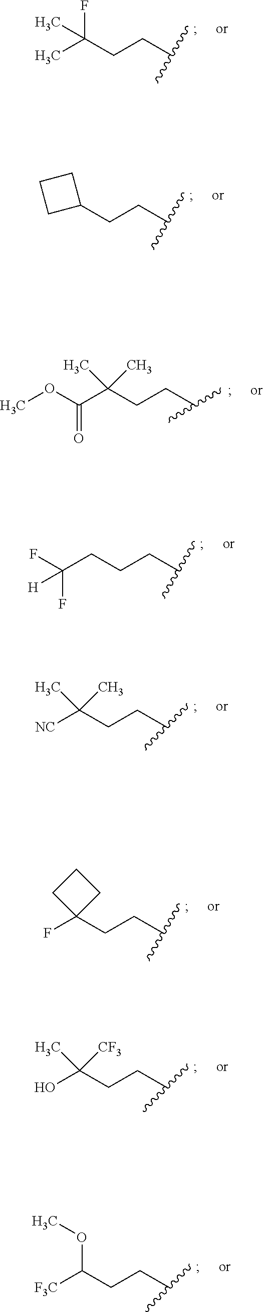

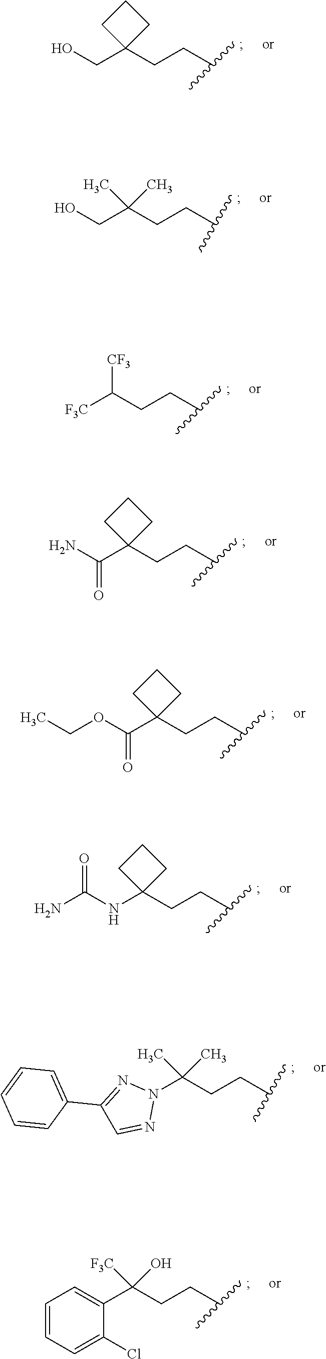

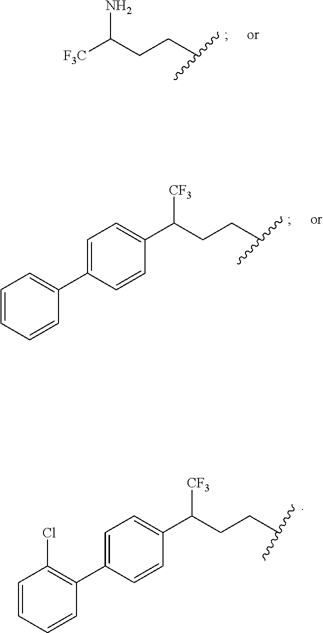 [SMILES: *CCC(N)C(F)(F)F ; *CCC(c1ccc(-c2ccccc2)cc1)C(F)(F)F ; *CCC(c1ccc(-c2ccccc2Cl)cc1)C(F)(F)F]

16. A compound, a diastereomer or an enantiomer of the compound, or a pharmaceutically acceptable salt of any of the foregoing, or a deuterated derivative of any of the foregoing; wherein the compound is selected from the group consisting of:

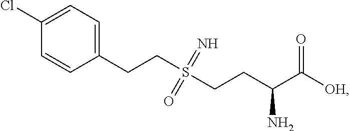 [SMILES: N=S(=O)(CCc1ccc(Cl)cc1)CC[C@H](N)C(=O)O]

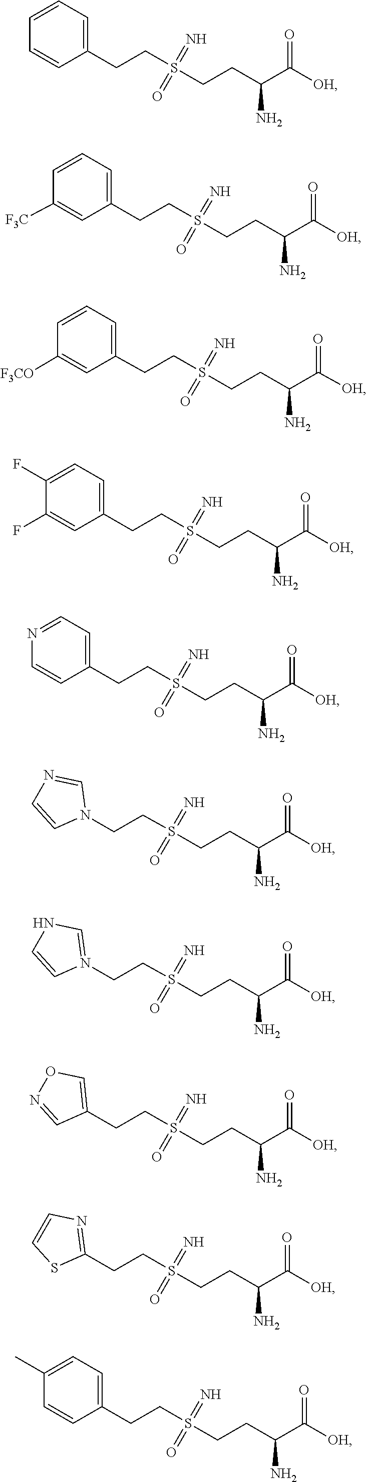

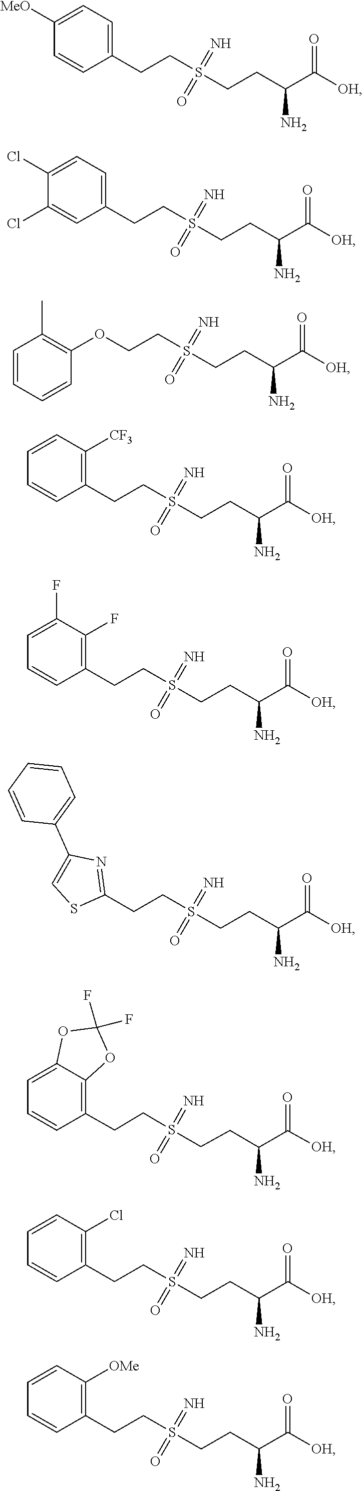

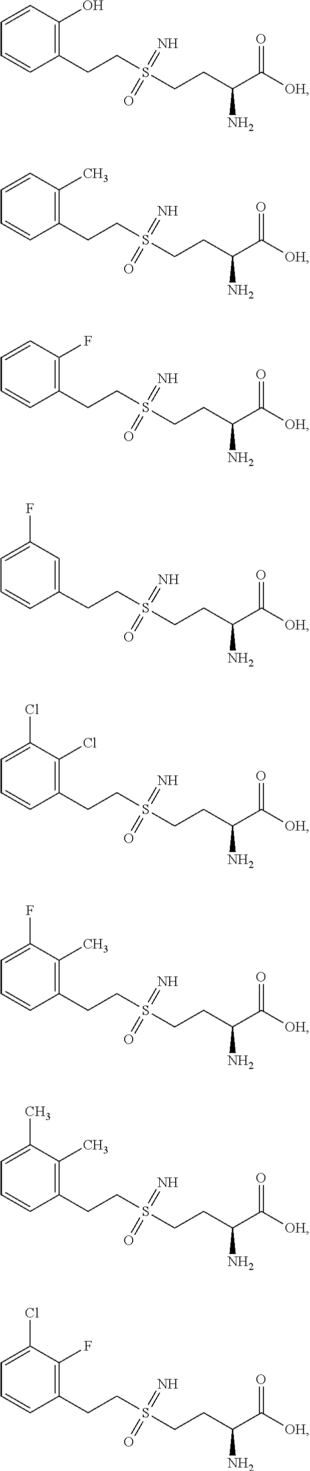

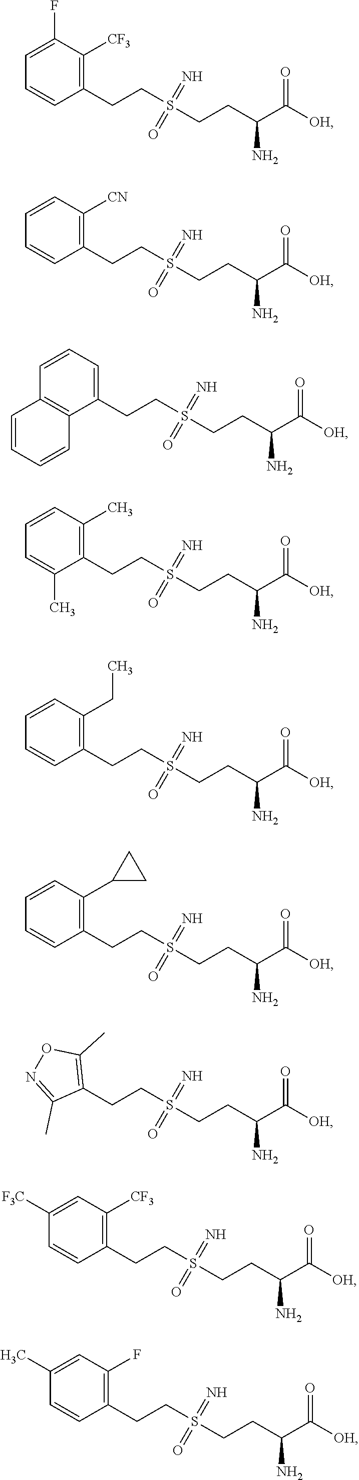

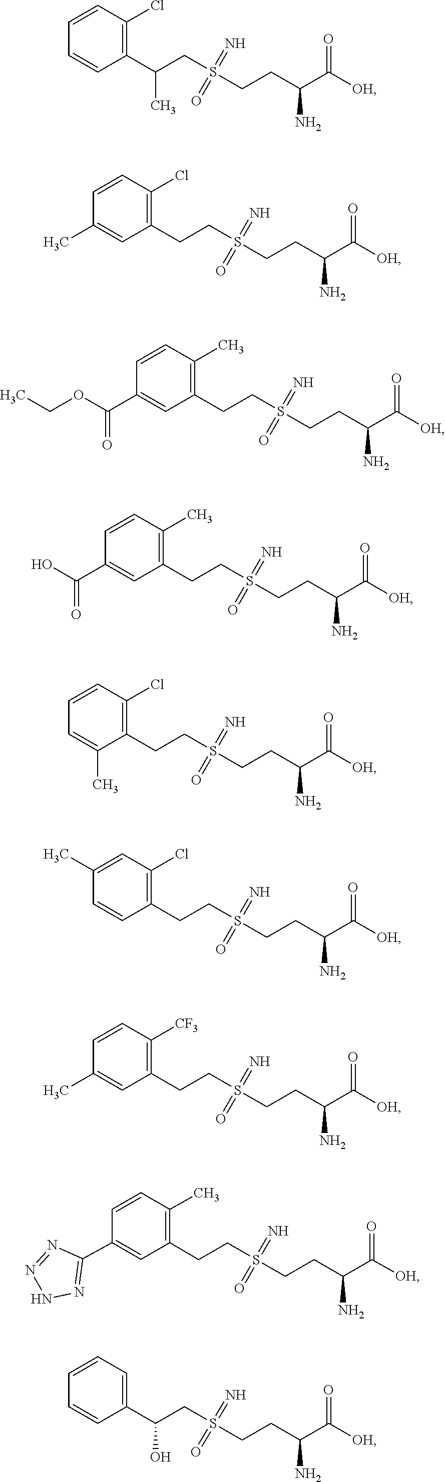

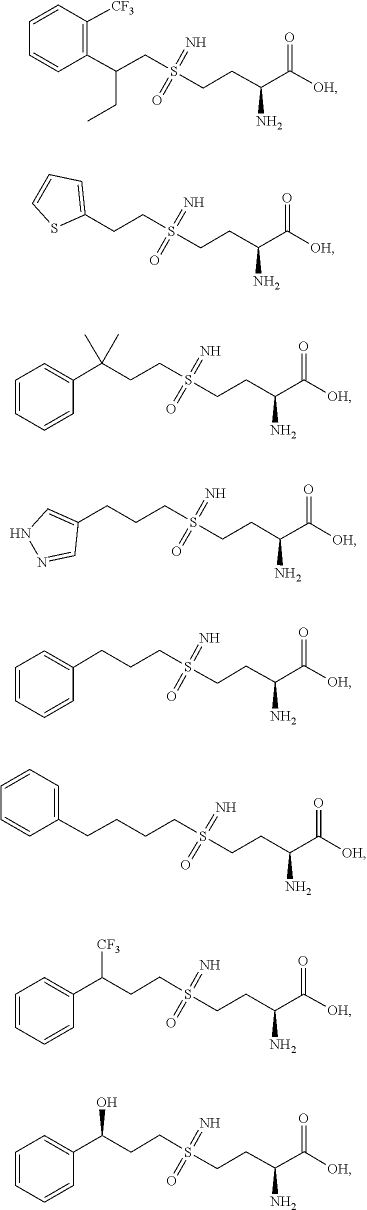

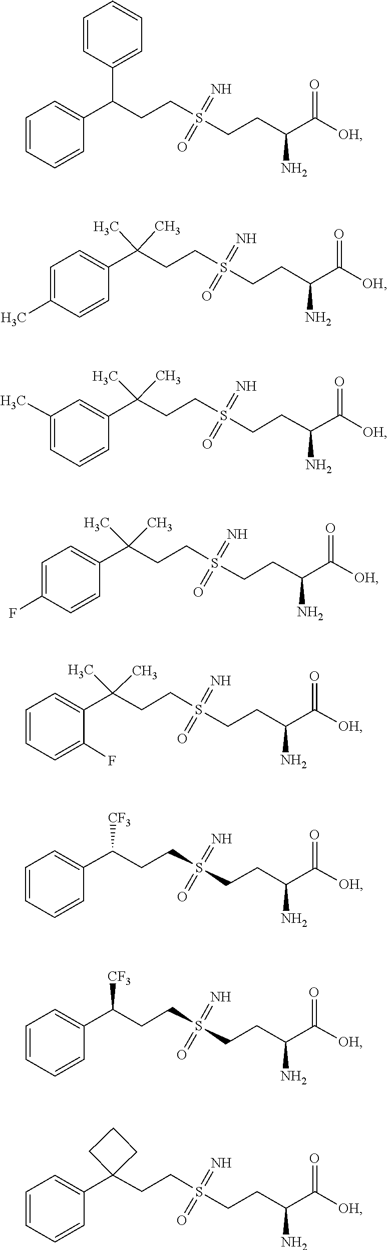

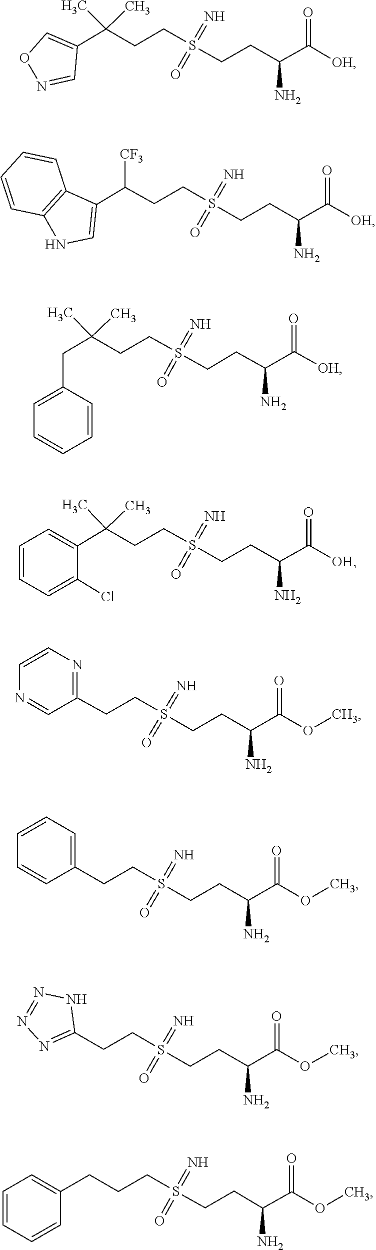

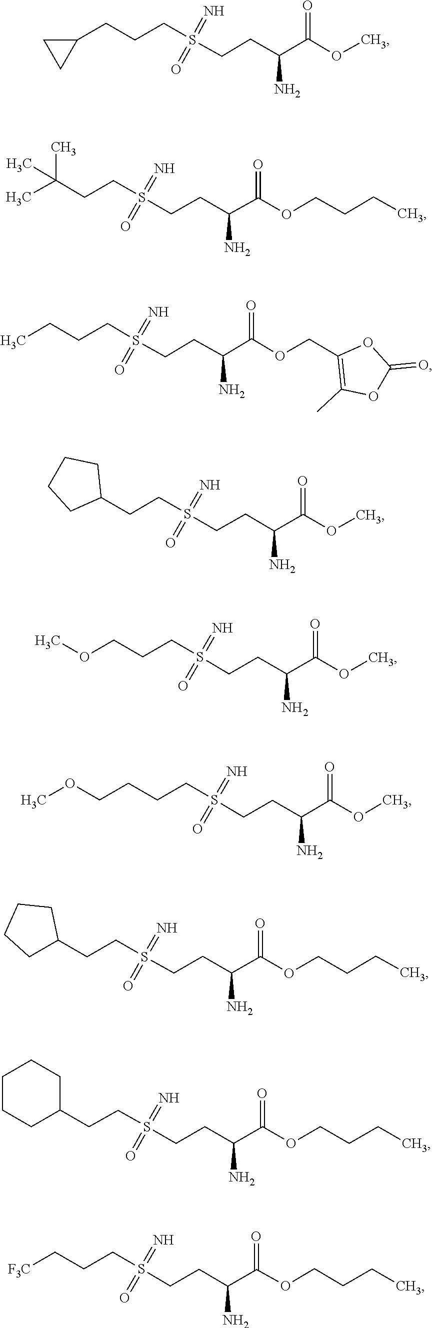

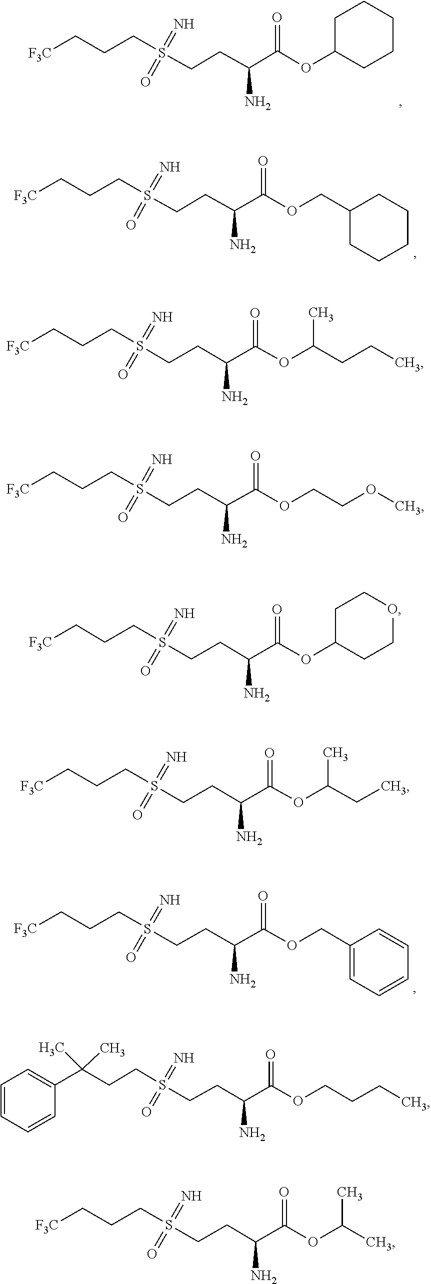

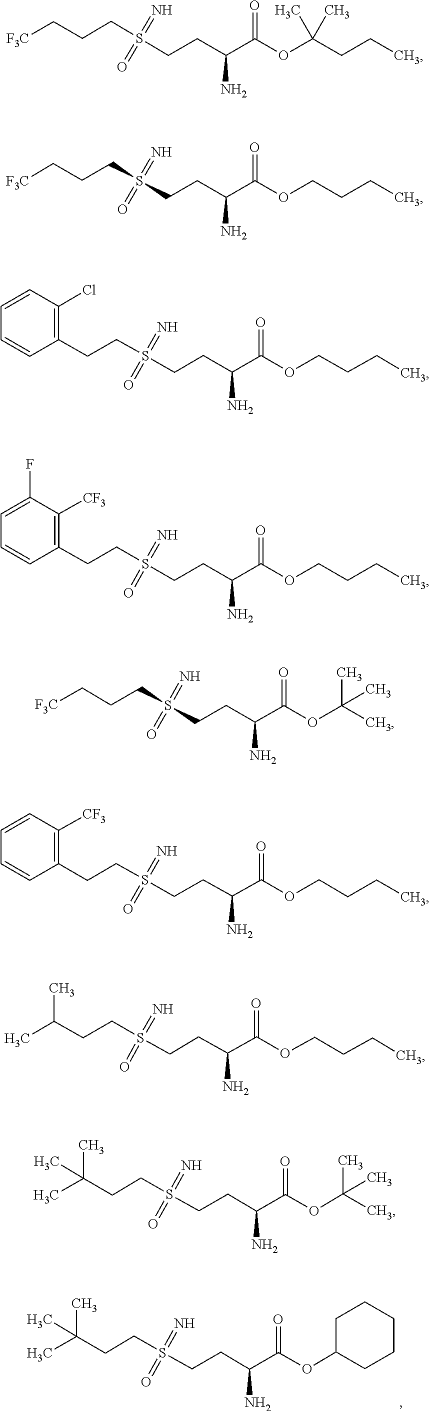

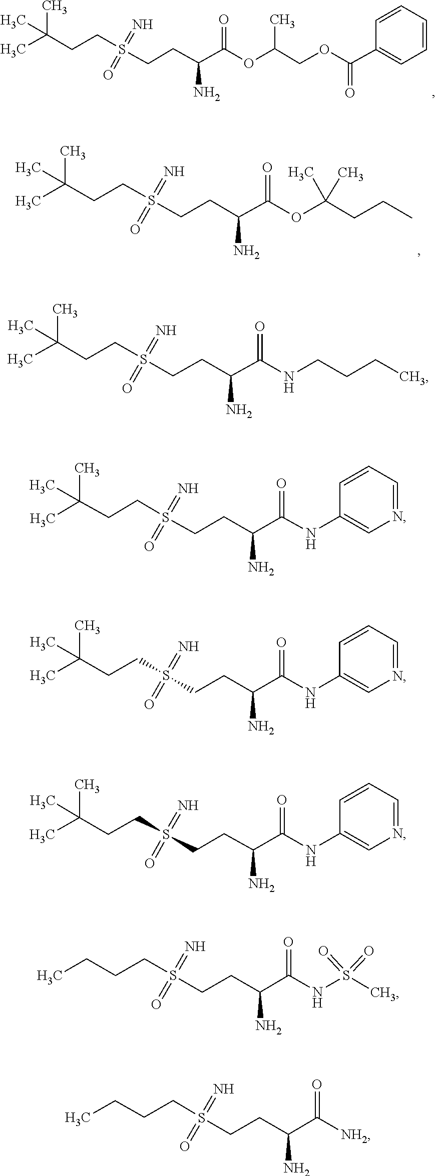

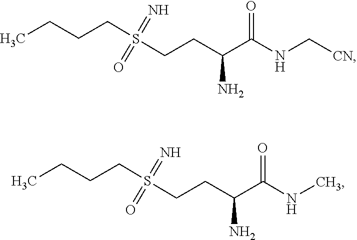 [SMILES: CCCCS(=N)(=O)CC[C@H](N)C(=O)NCC#N ; CCCCS(=N)(=O)CC[C@H](N)C(=O)NC]

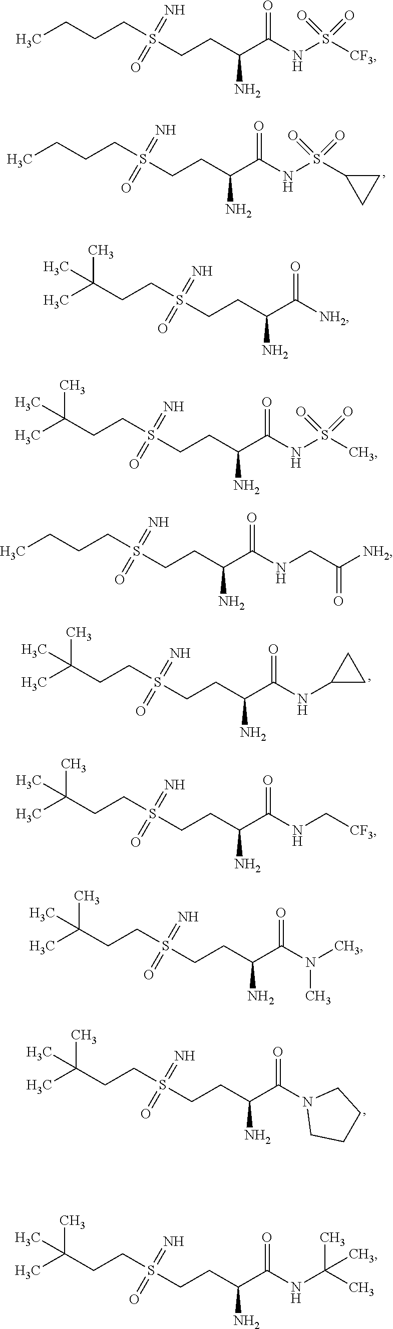

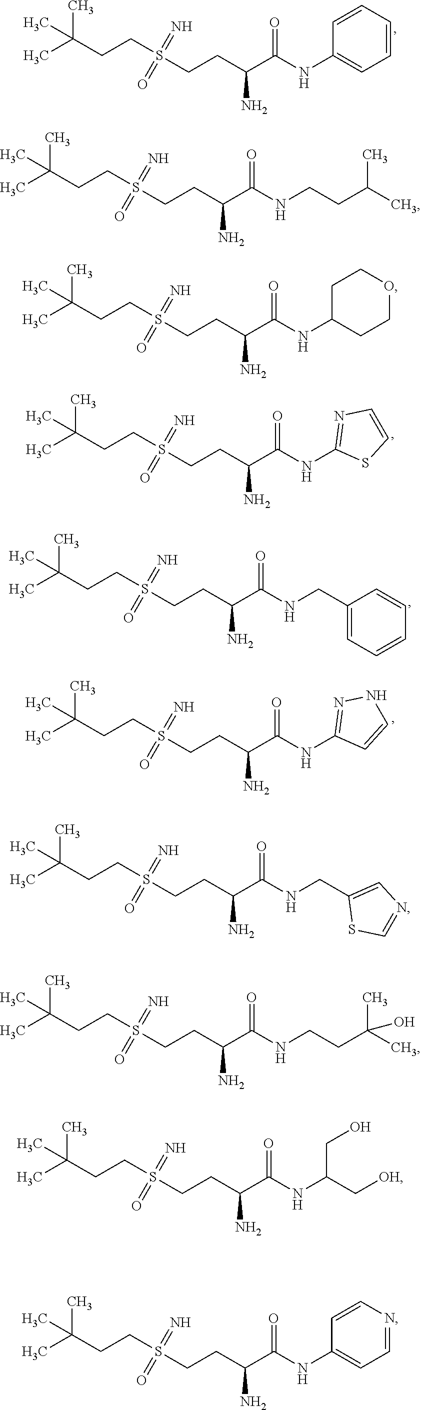

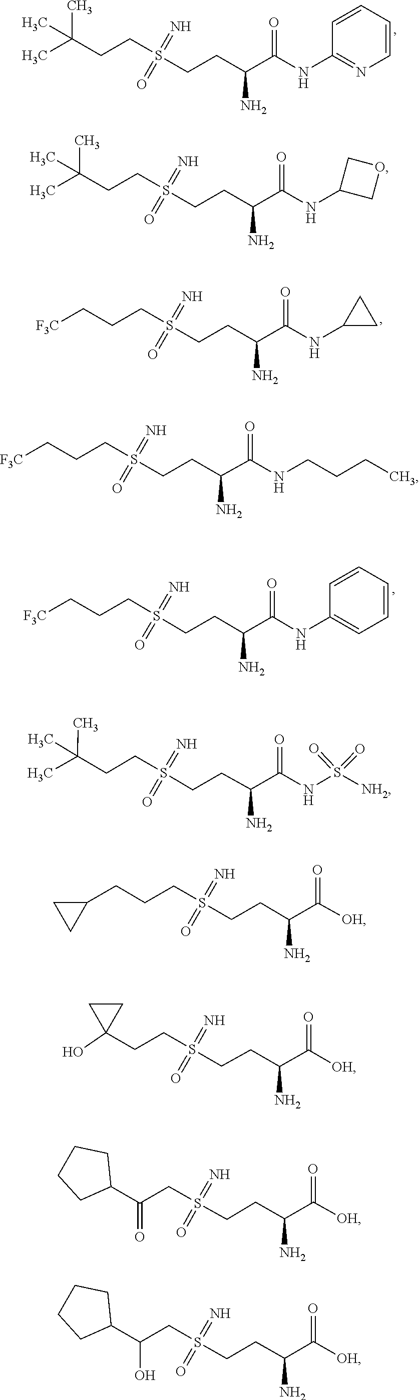

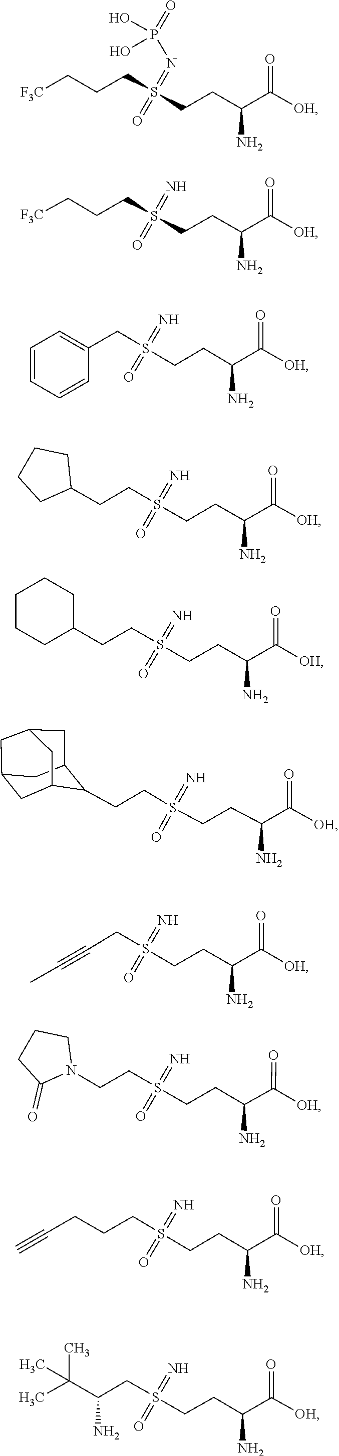

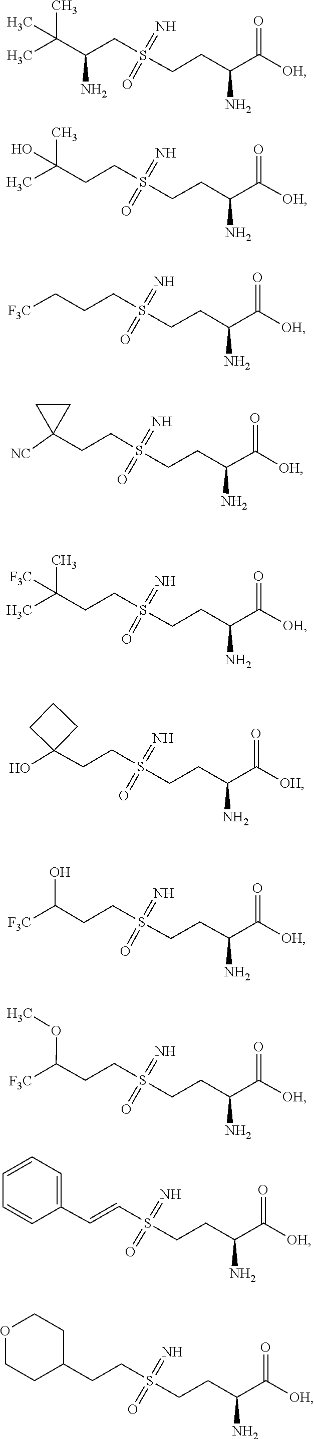

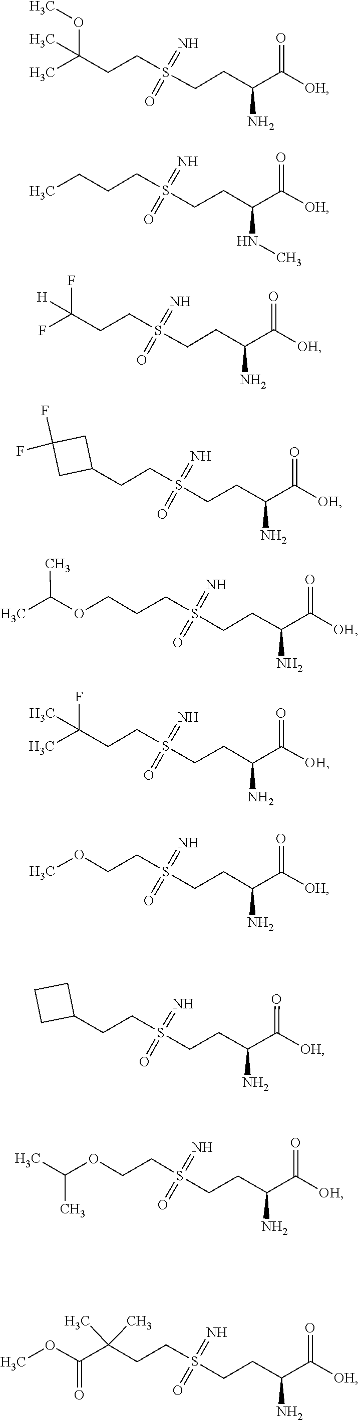

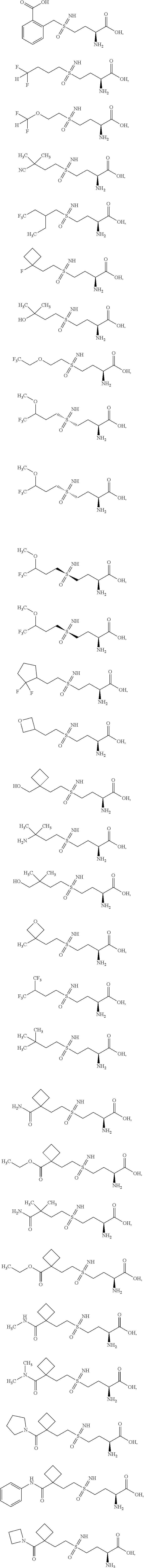

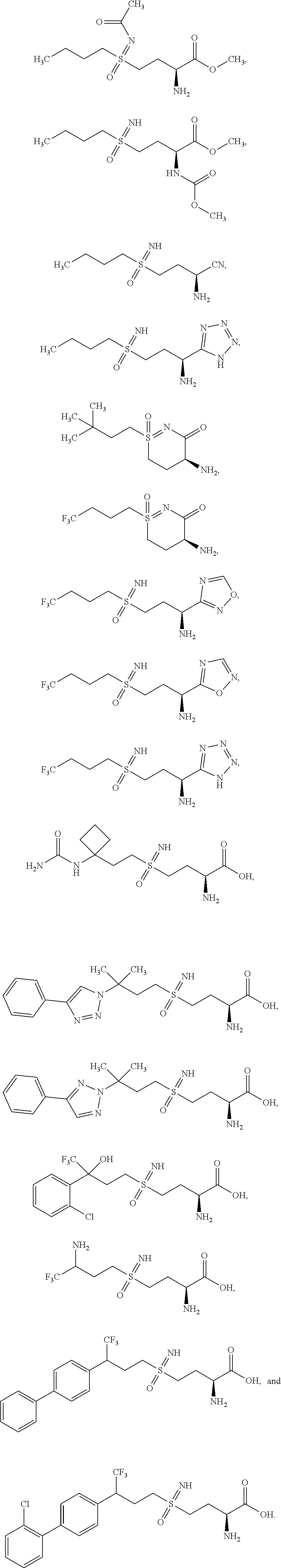

17. A compound, a diastereomer or an enantiomer of the compound, or a pharmaceutically acceptable salt of any of the foregoing, or a deuterated derivative of any of the foregoing; w herein the compound is selected from the group consisting of:

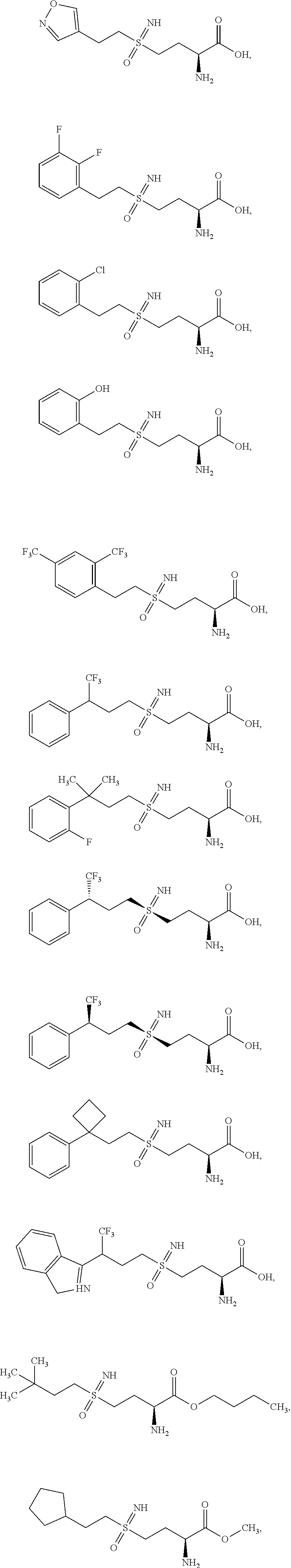

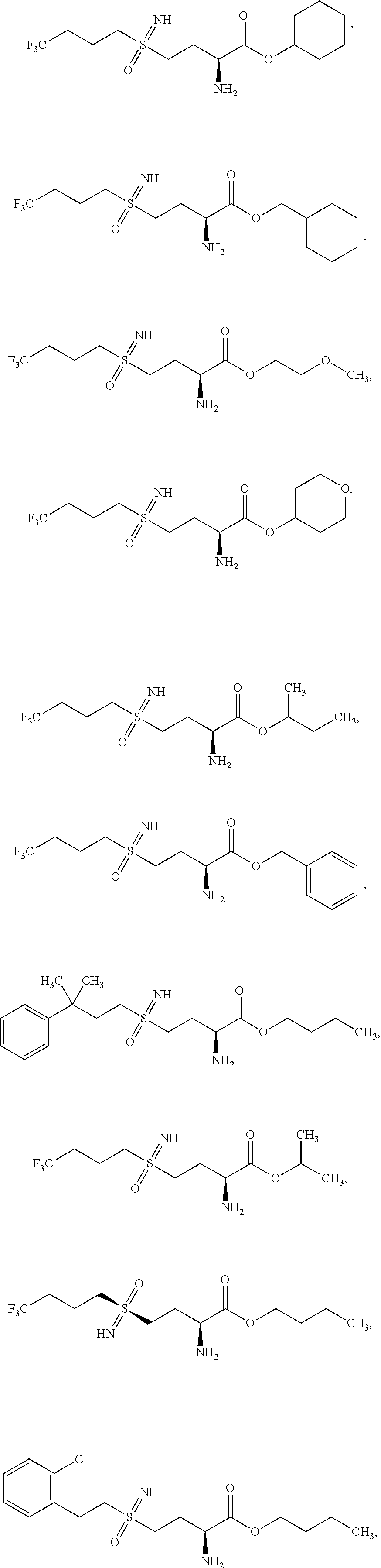

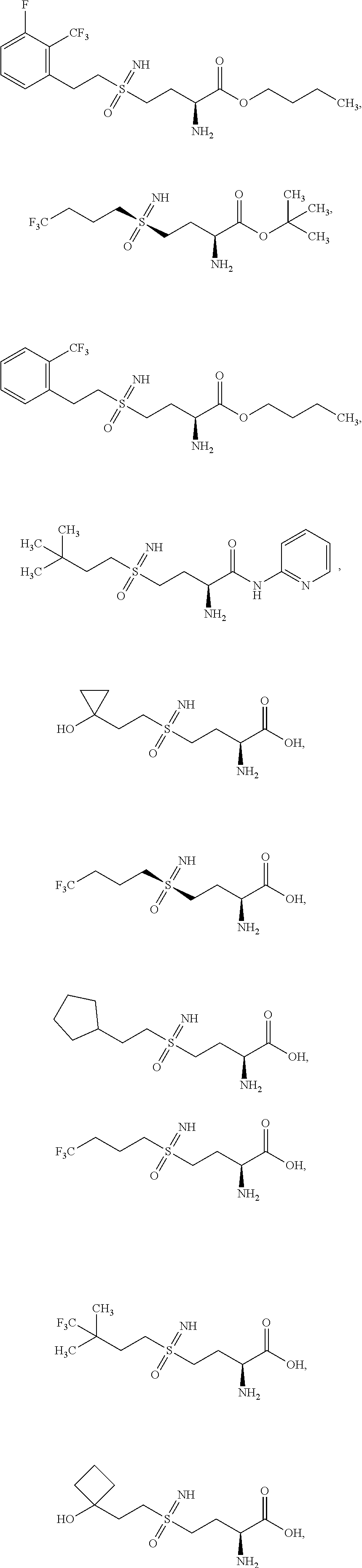

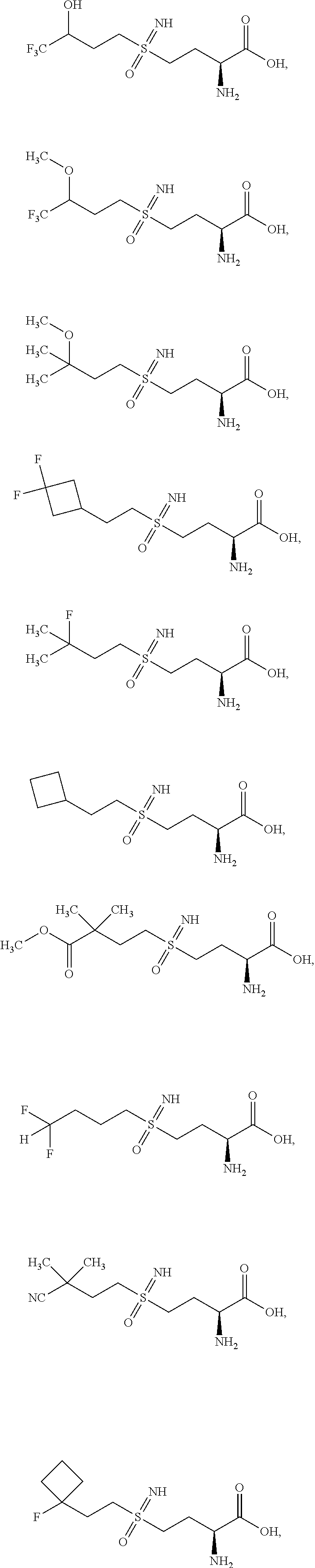

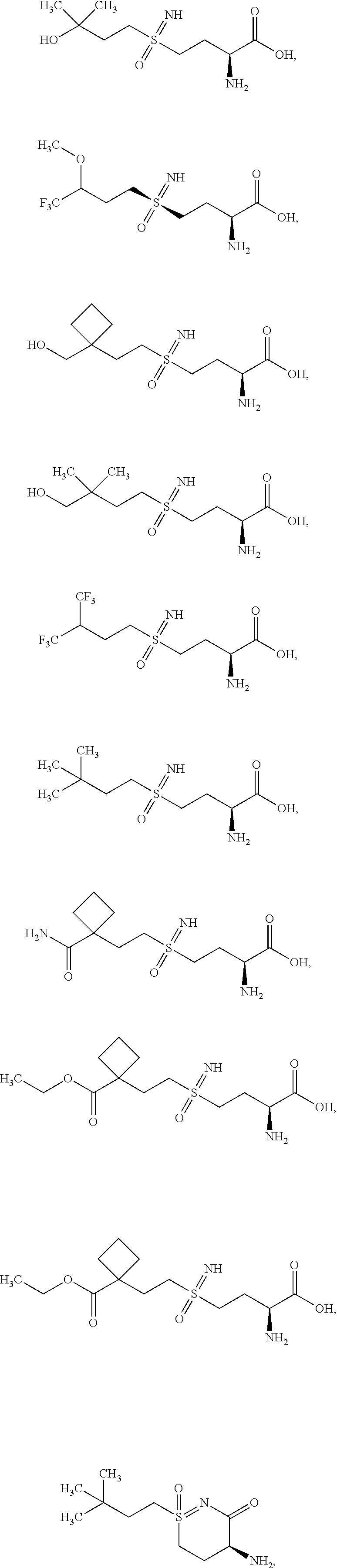

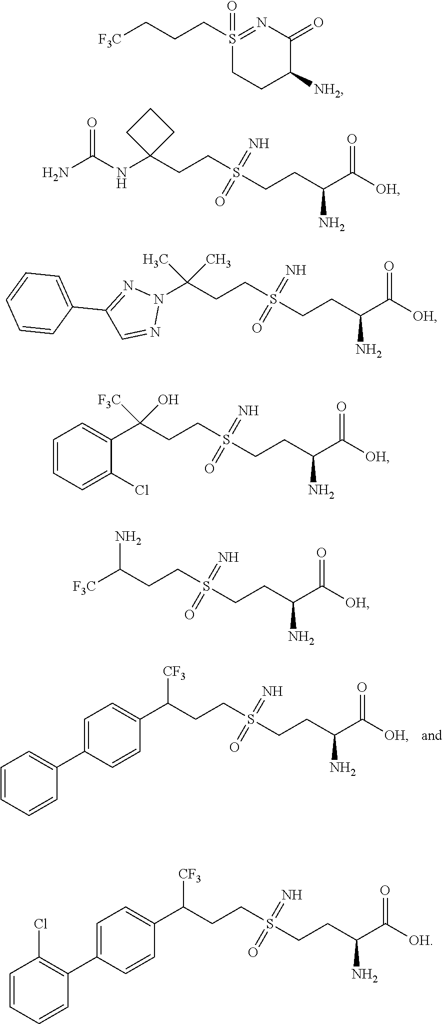

18. A compound of Formula II 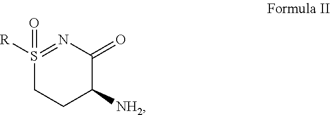
a diastereomer or an enantiomer of the compound of Formula II, or a pharmaceutically acceptable salt of any of the foregoing, or a deuterated derivative of any of the foregoing; wherein in the compound of Formula II, R is a: C₁, C₂, C₃, C₄, C₅, C₆, C₇, C₈, C₉, or C₁₀ linear or branched chain alkyl, optionally and independently substituted by a substituent selected from the group consisting of: a deuterium, a halogen, a fluorine, a chlorine, a C₁-C₁₀ linear or branched chain alkyl, a methyl, an ethyl, a propyl, an iso-propyl, a butyl, an isobutyl, a sec-butyl, a tert-butyl, and any combination of these.

19. A pharmaceutical composition comprising the compound of Formula I, the compound of Formula II, or the compound, of any one of claims 1-18; the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier.

20. The pharmaceutical composition of claim 19, that is in unit dose form.

21. The pharmaceutical composition of any one of claims 19-20, that further comprises an additional active agent or a pharmaceutically acceptable salt thereof.

22. The pharmaceutical composition of claims 19-21, that is in the form of a powder, a tablet, a capsule, a liquid, or a gel.

23. The pharmaceutical composition of any one of claims 19-22, wherein the compound of Formula I, the compound of Formula II, the compound; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg.

24. A kit comprising the pharmaceutical composition of any one of claims 19-23 and a container.

25. The kit of claim 24, wherein the container is a syringe.

26. The kit of claim 24, wherein the container is an IV bag.

27. The kit of claim 24, wherein the container is disposable.

28. The kit of claim 24, wherein the container is a single use container.

29. The kit of claim 24, wherein the container is a resealable container.

30. A method of treating a cancer in a subject, the method comprising administering the pharmaceutical composition of any one of claims 19-23 to the subject in a therapeutically effective amount, thereby treating the cancer.

31. A method of treating a cancer in a subject, the method comprising administering to the subject the compound of Formula I, the compound of Formula II, or the compound, of any one of claims 1-18; the diastereomer or the enantiomer of any of the foregoing, or the pharmaceutically acceptable salt of any of the foregoing, or the deuterated derivative of any of the foregoing; in a therapeutically effective amount, thereby treating the cancer.

32. The method of claim 30 or claim 31, wherein the administering is selected from the group consisting of: oral, an injection; subcutaneous, intra-tumoral; systemic, local, intravenous, intraperitoneal, intramuscular, and any combination thereof.

33. A method of modulating ferroptosis in a tissue, the method comprising contacting the tissue with the pharmaceutical composition of any one of claims 19-23 in an amount effective to modulate the ferroptosis in the tissue.

34. The method of claim 33, wherein the tissue is comprised in a subject.

35. The method of any one of claims 30-32 or 34, wherein the subject is a subject in need thereof.

36. The method of claim 34 or 35, wherein the subject is a mammal.

37. The method of claim 36, wherein the subject is a human.

38. The method of any one of claims 30-37, wherein the administering or the contacting is: as needed, once per day, twice per day, three times per day, once per week, once per two weeks, once per three weeks, once per month, once every six months, once per year, or for life.

39. The method of any one of claims 30-38, wherein the therapeutically effective amount, or the amount effective, ranges from about 0.001 mg to about 25,000 mg.

40. A compound, a diastereomer or an enantiomer of the compound, or a pharmaceutically acceptable salt of any of the foregoing, or a deuterated derivative of any of the foregoing; wherein the compound is selected from the group consisting of:

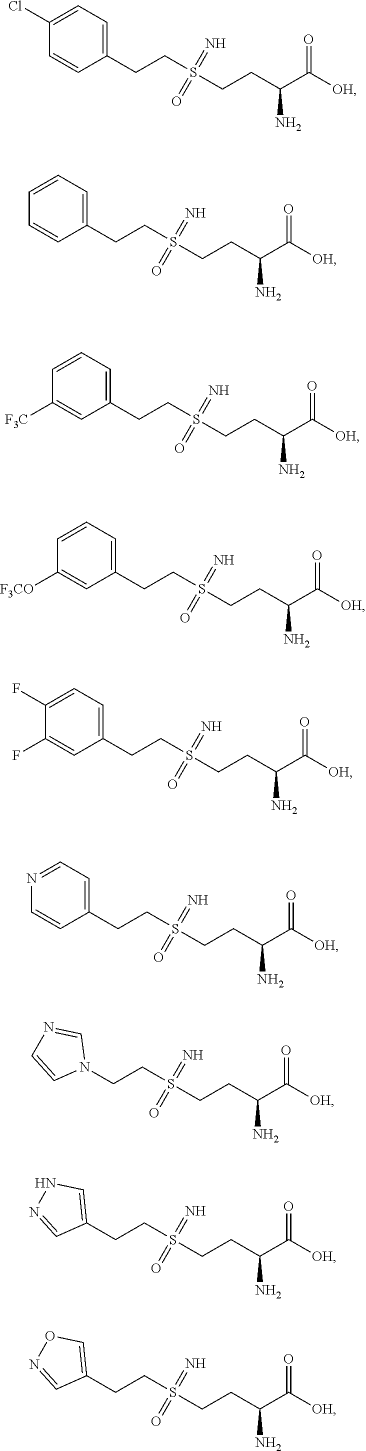

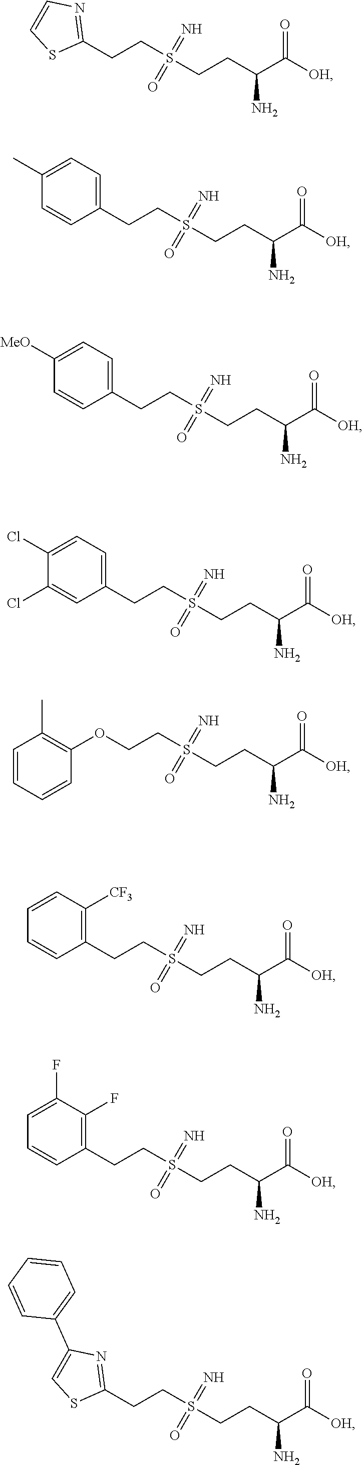

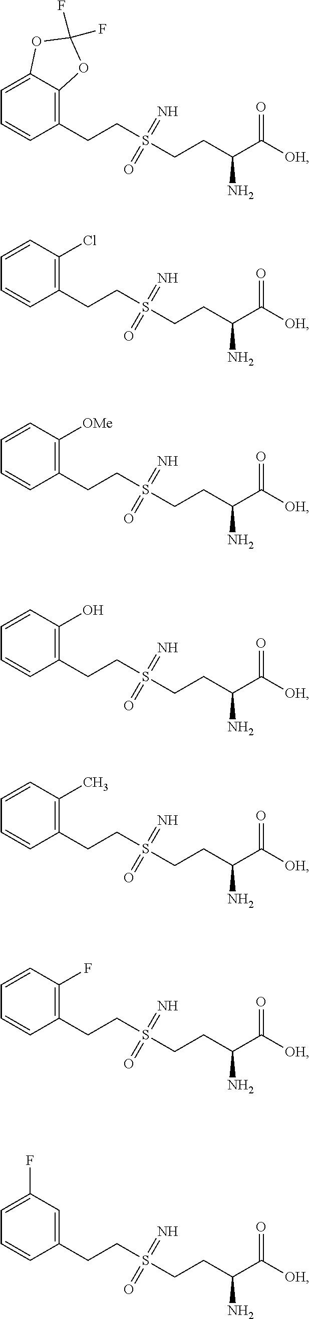

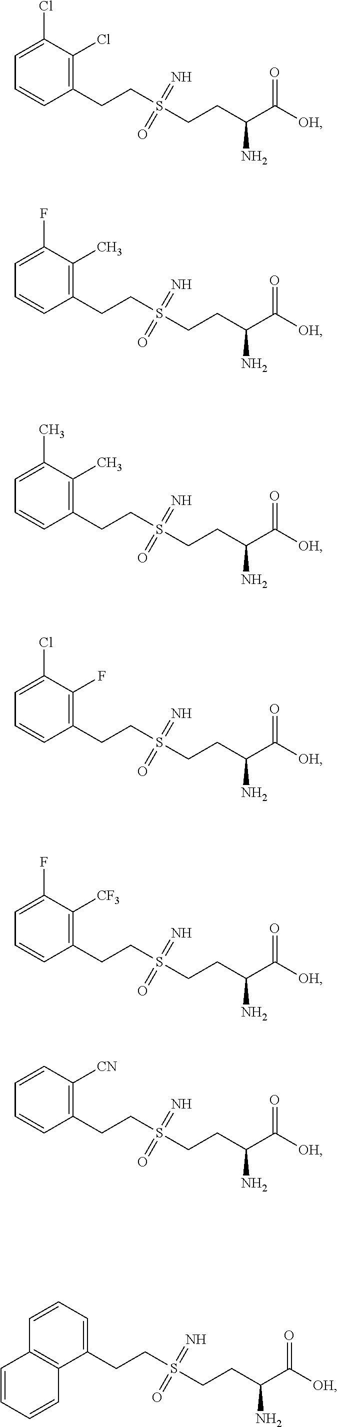

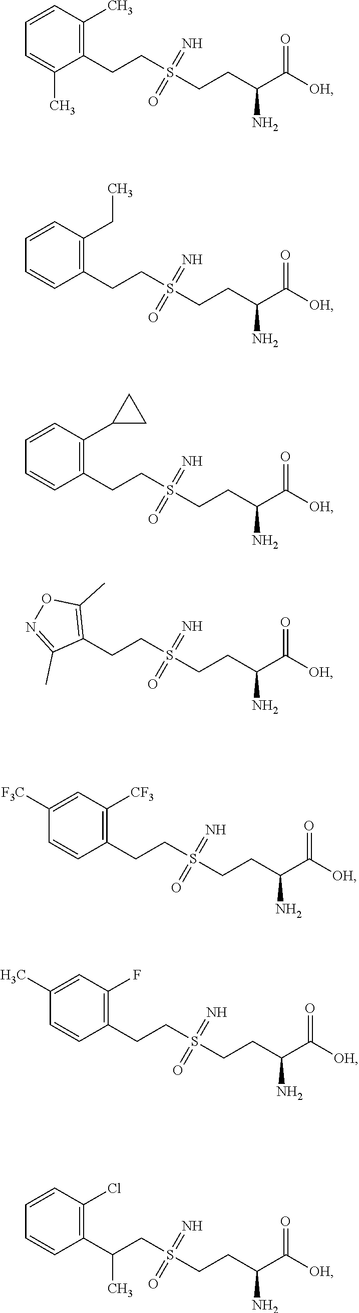

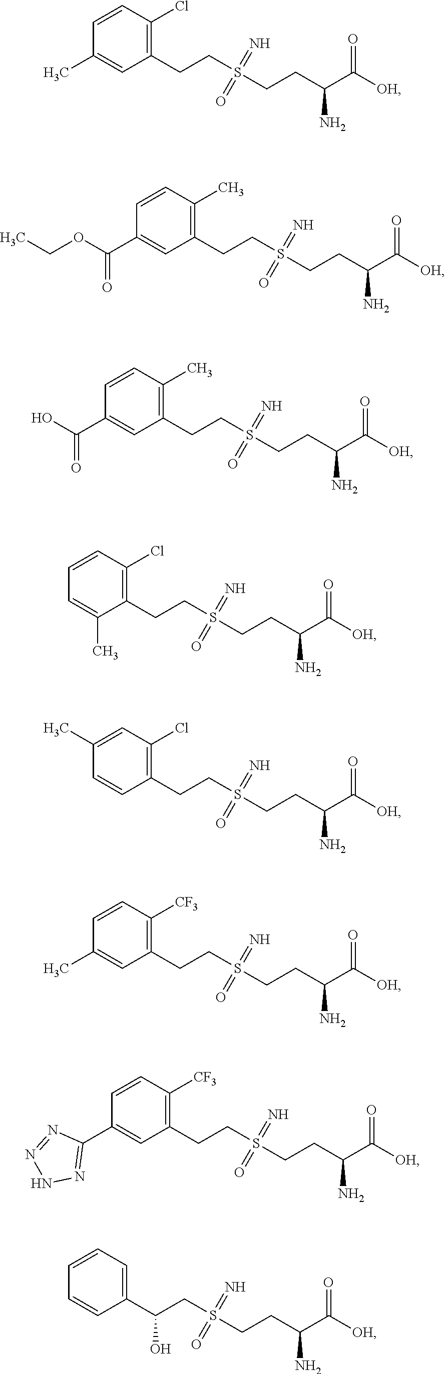

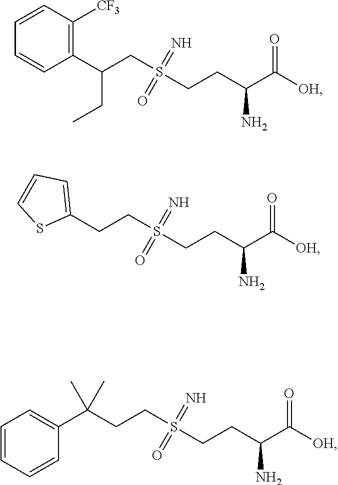 [SMILES: CCC(CS(=N)(=O)CC[C@H](N)C(=O)O)c1ccccc1C(F)(F)F ; N=S(=O)(CCc1cccs1)CC[C@H](N)C(=O)O ; CC(C)(CCS(=N)(=O)CC[C@H](N)C(=O)O)c1ccccc1]

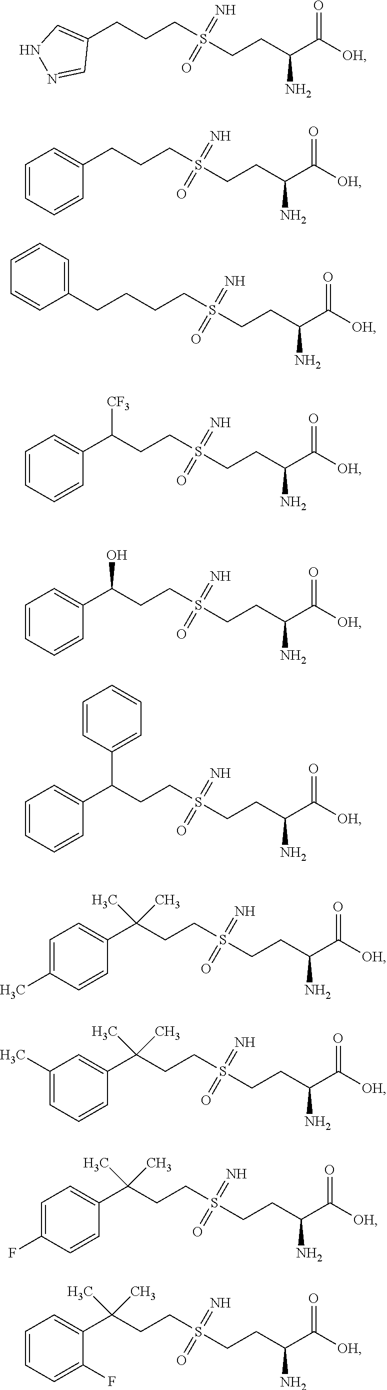

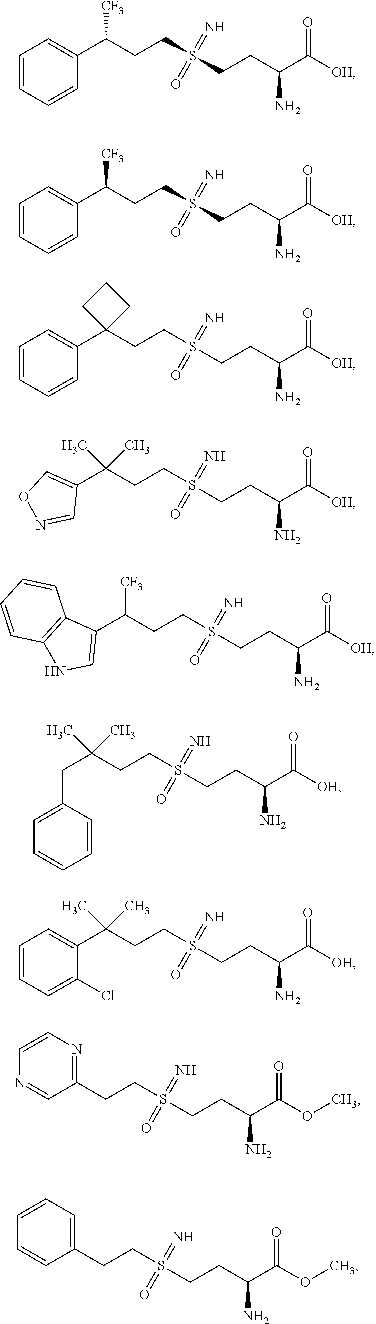

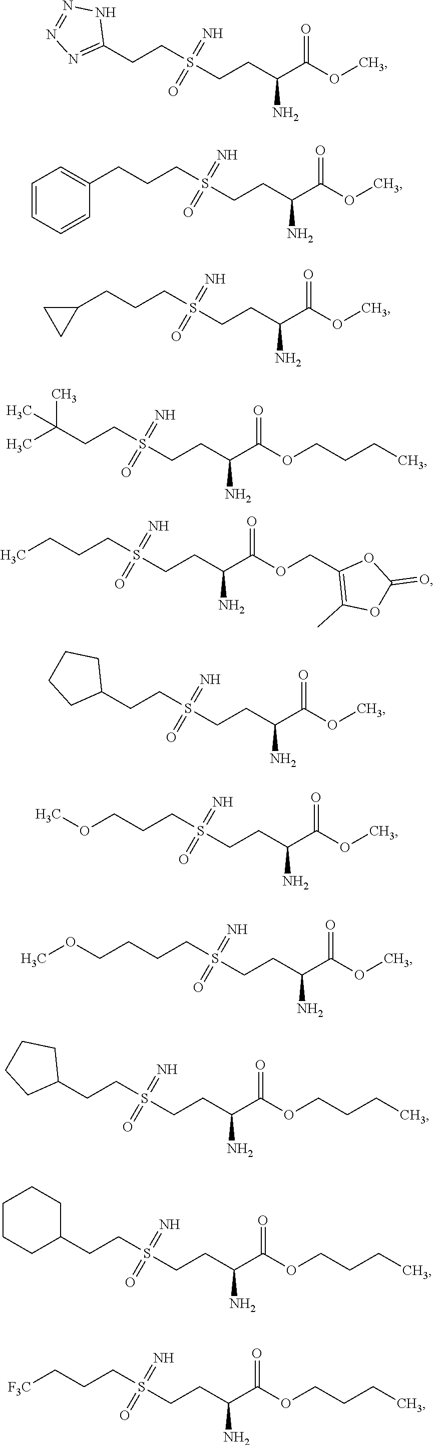

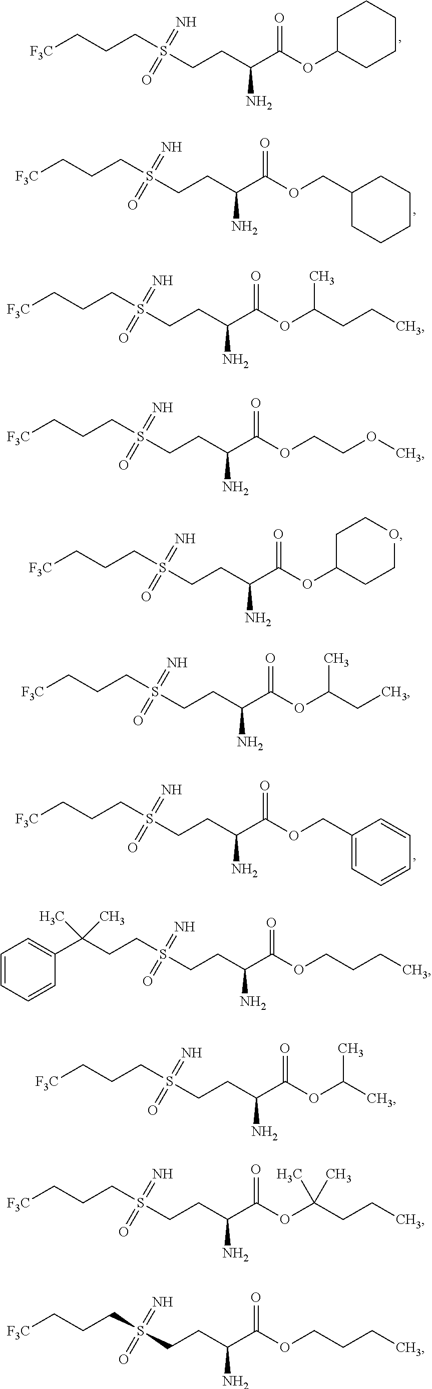

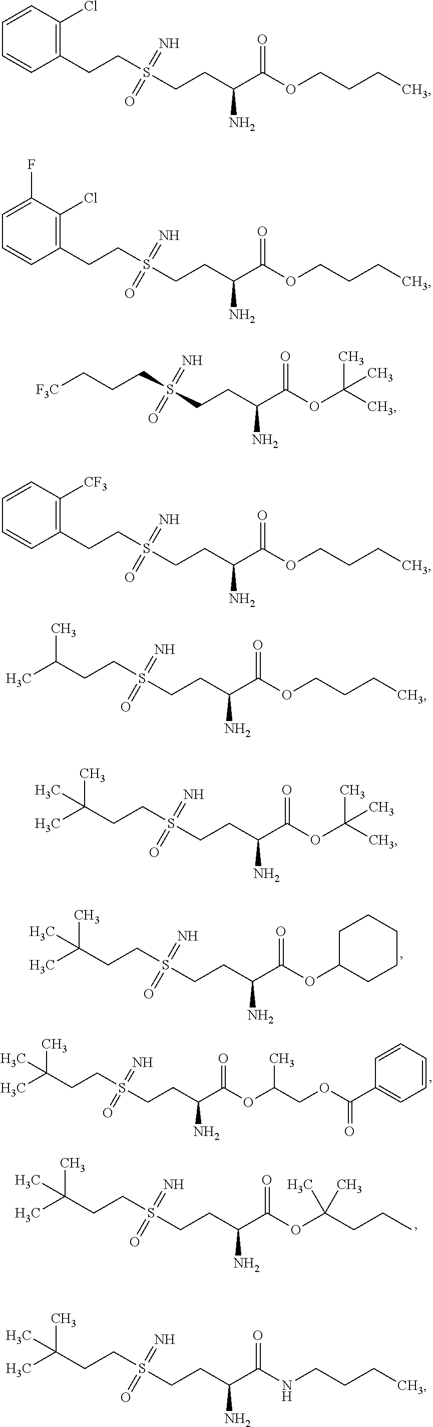

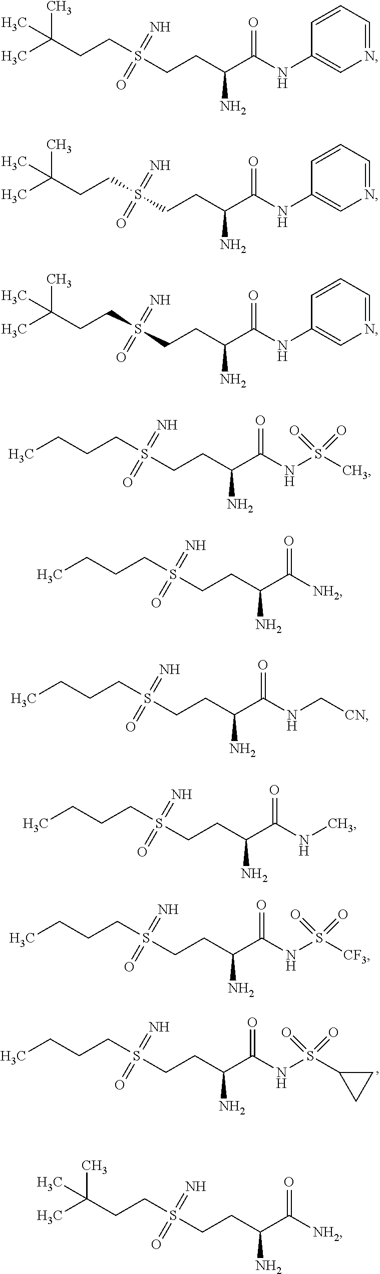

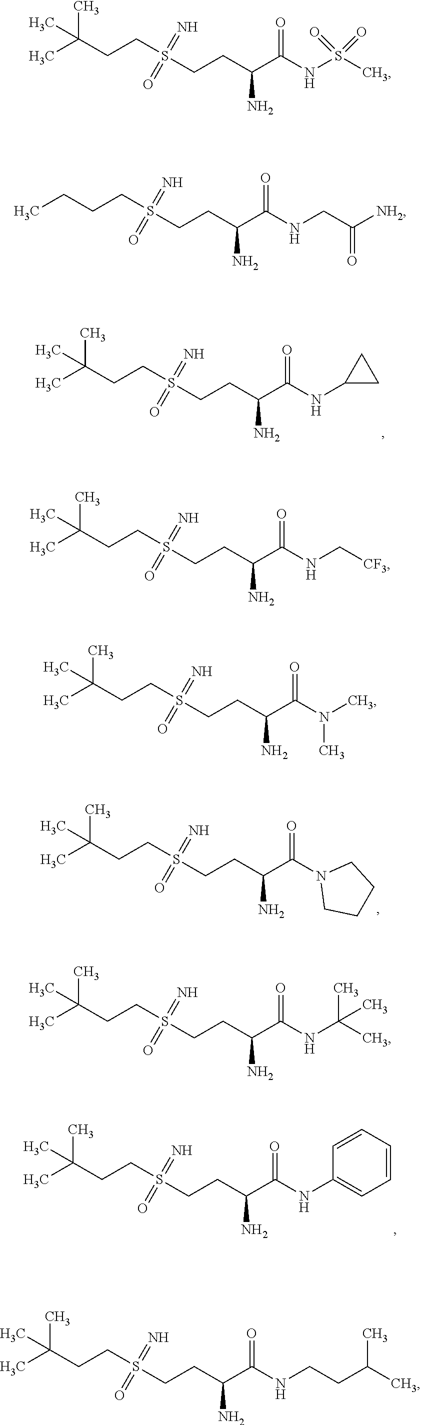

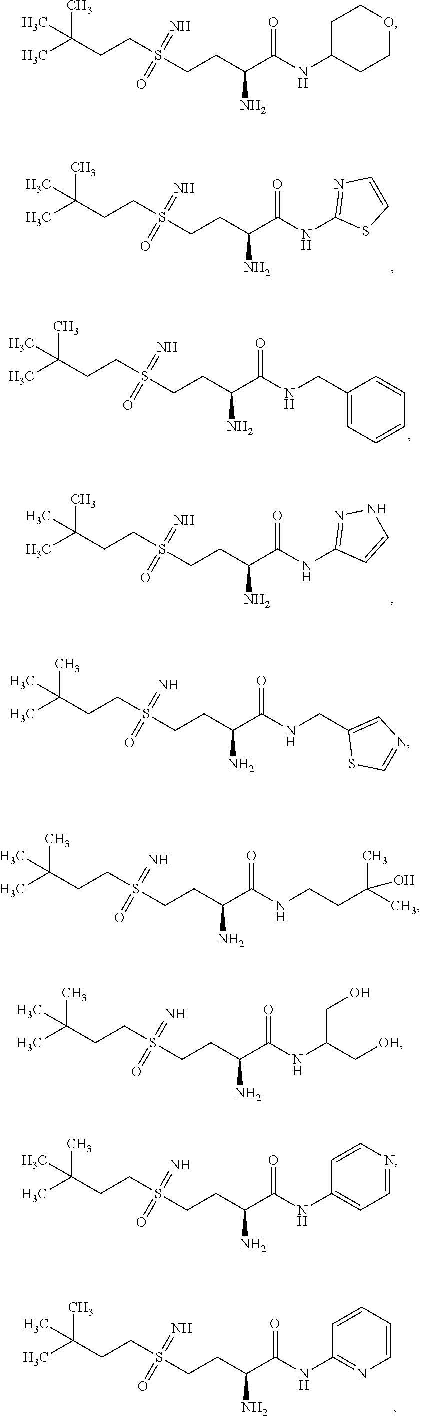

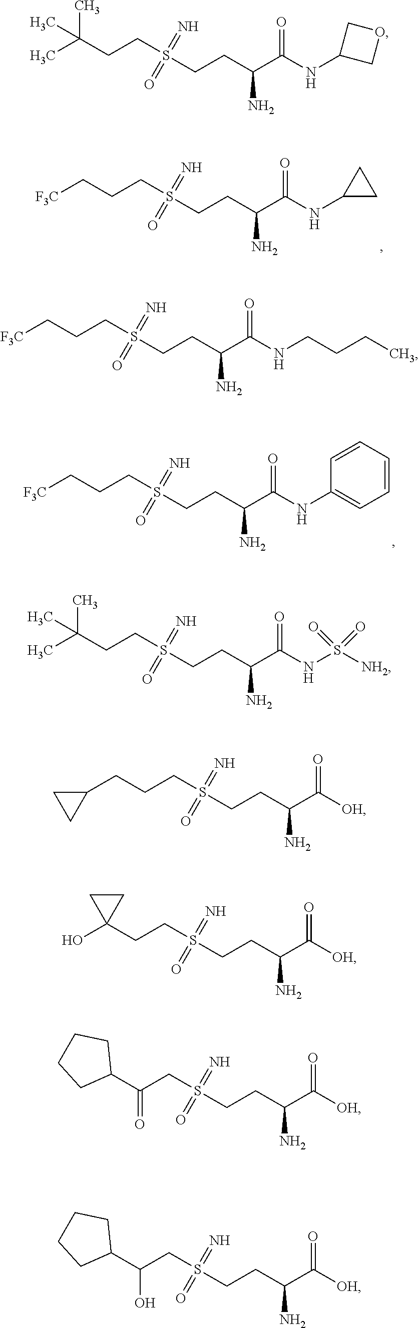

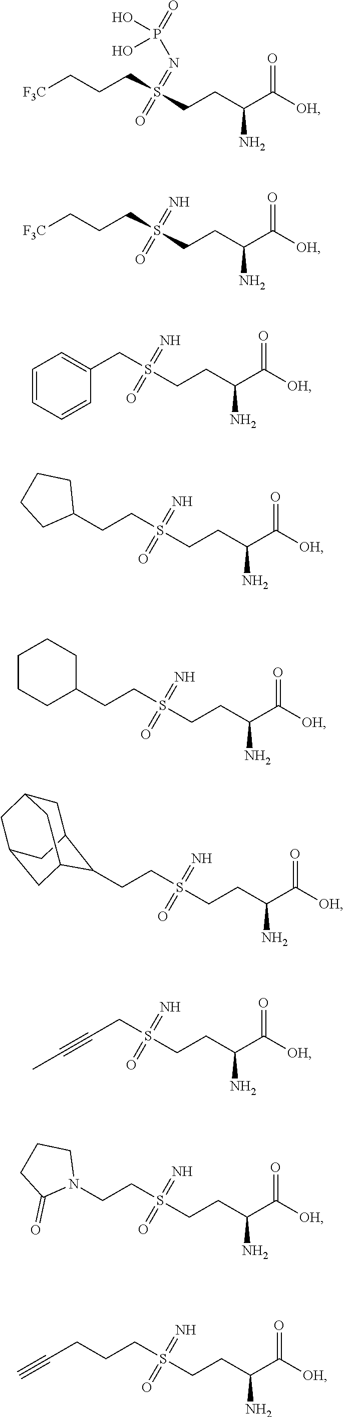

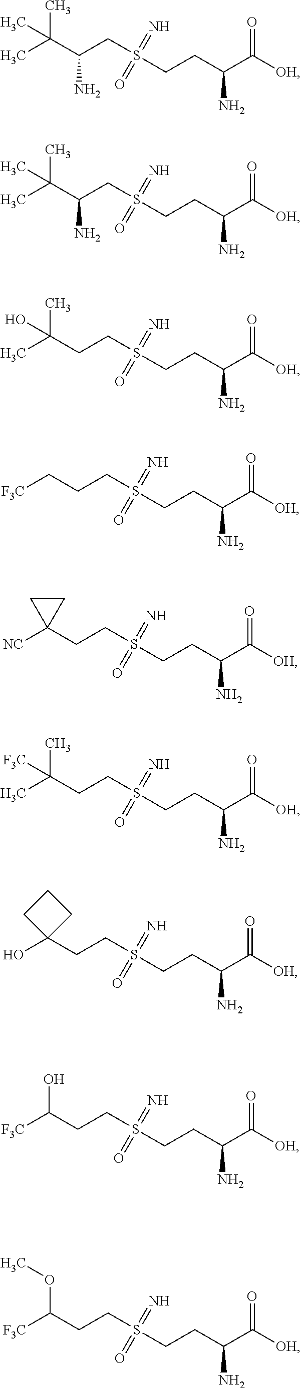

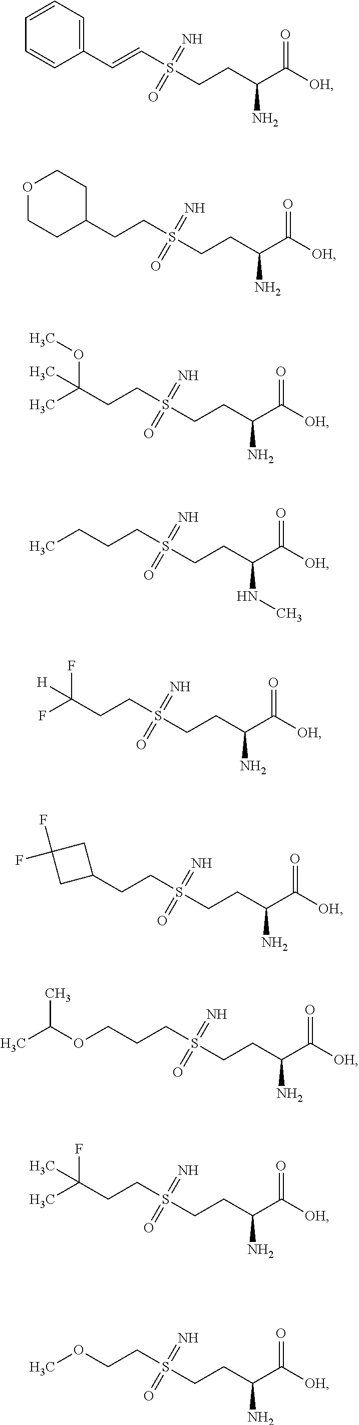

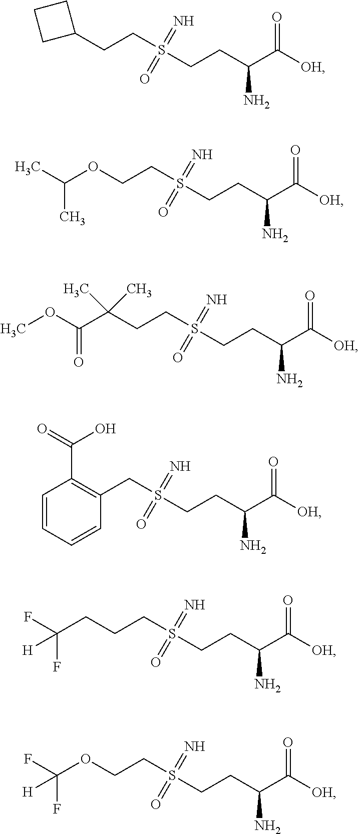 [SMILES: N=S(=O)(CCC1CCC1)CC[C@H](N)C(=O)O ; CC(C)OCCS(=N)(=O)CC[C@H](N)C(=O)O ; COC(=O)C(C)(C)CCS(=N)(=O)CC[C@H](N)C(=O)O ; N=S(=O)(CC[C@H](N)C(=O)O)Cc1ccccc1C(=O)O ; N=S(=O)(CCCC(F)F)CC[C@H](N)C(=O)O ; N=S(=O)(CCOC(F)F)CC[C@H](N)C(=O)O]

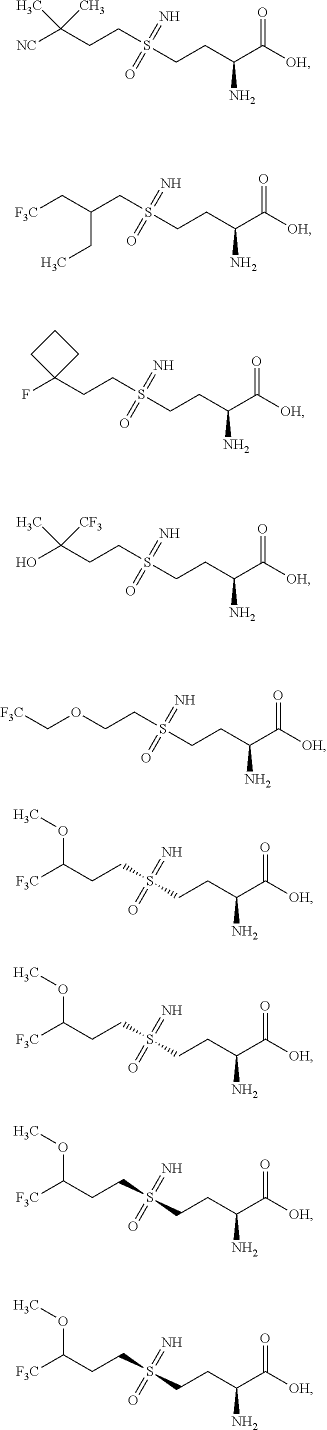

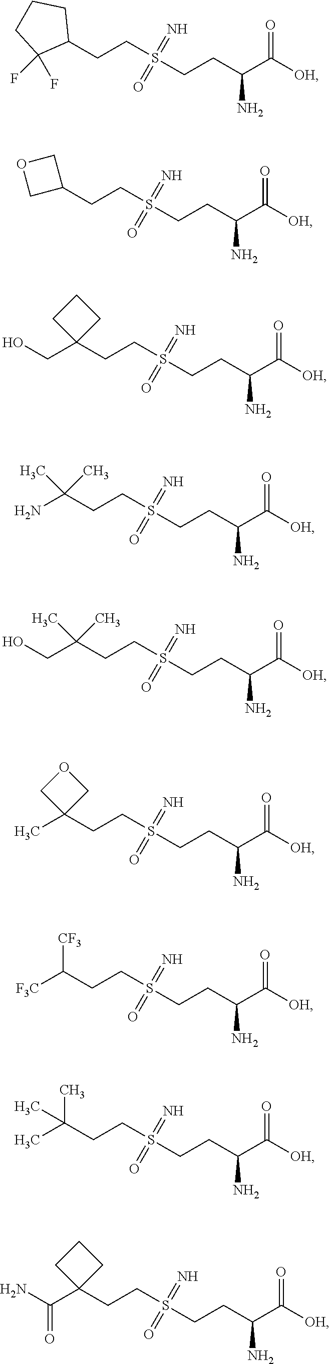

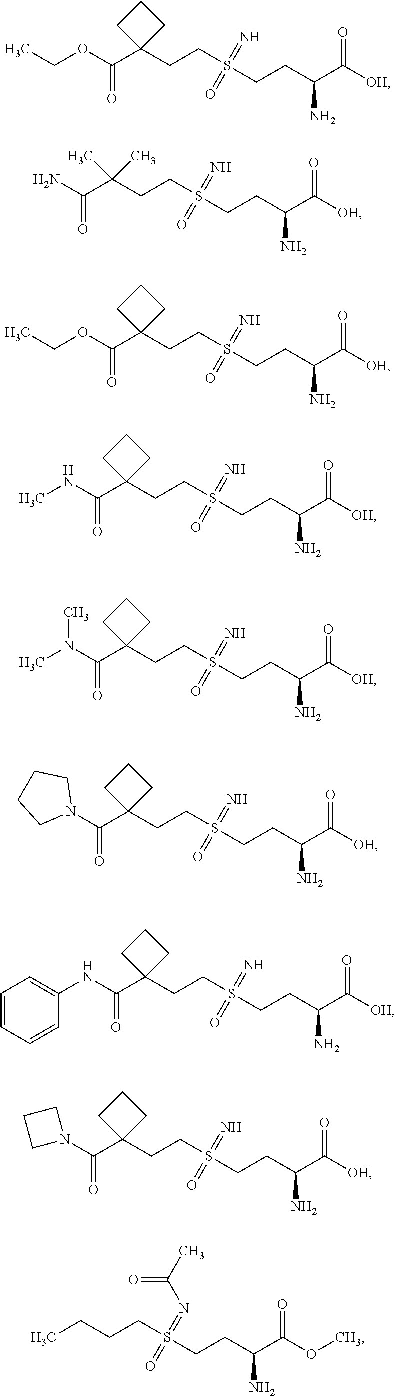

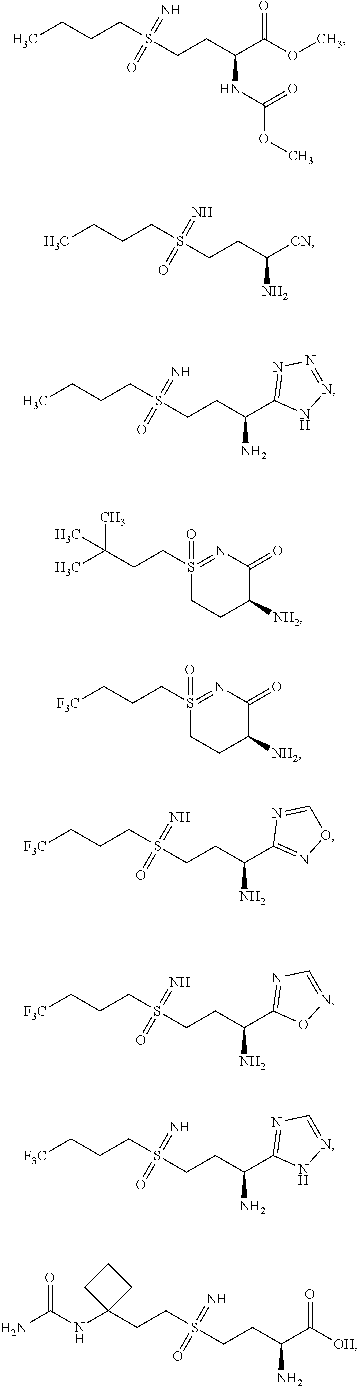

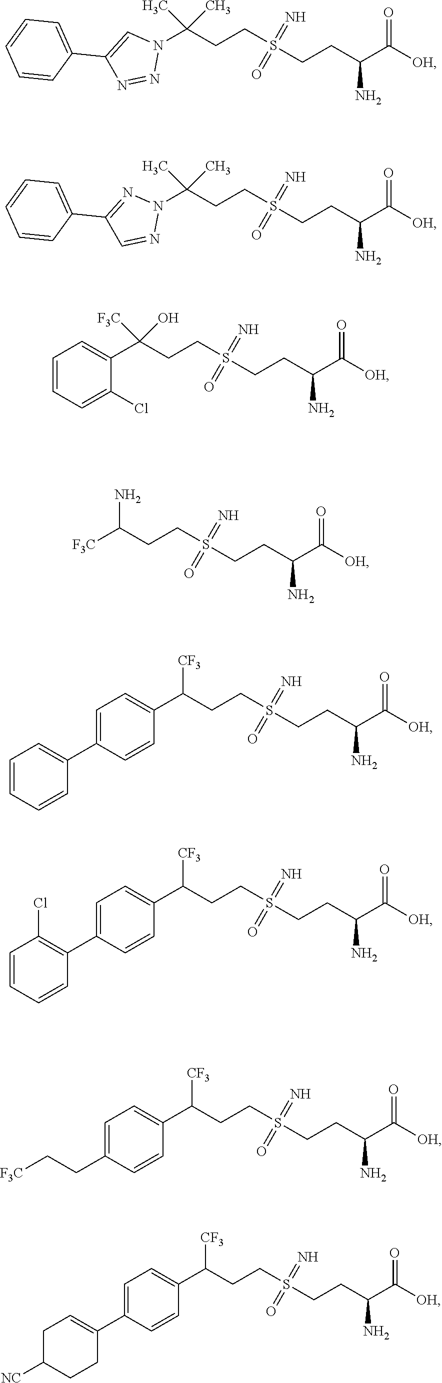

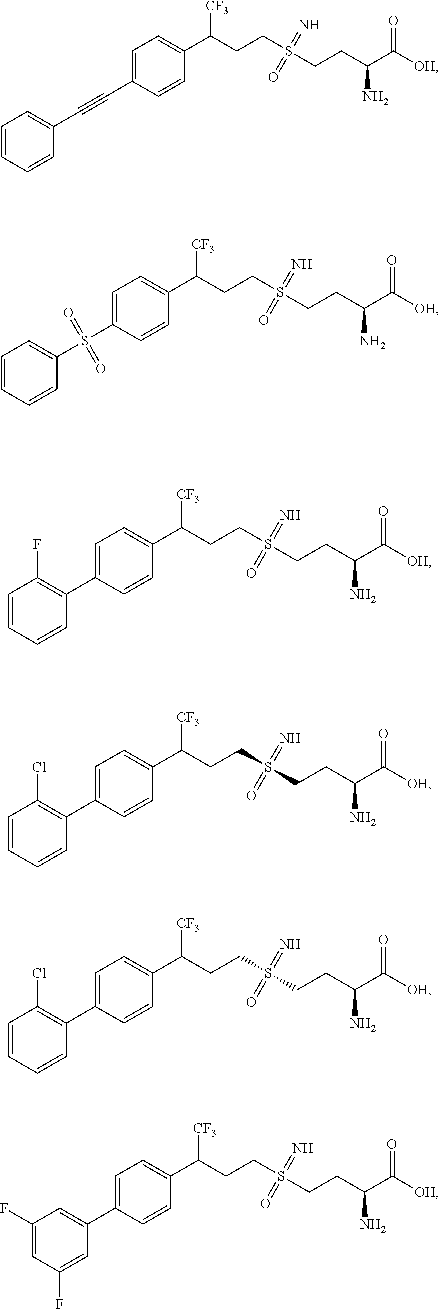 [SMILES: N=S(=O)(CCC(c1ccc(C#Cc2ccccc2)cc1)C(F)(F)F)CC[C@H](N)C(=O)O ; N=S(=O)(CCC(c1ccc(S(=O)(=O)c2ccccc2)cc1)C(F)(F)F)CC[C@H](N)C(=O)O ; N=S(=O)(CCC(c1ccc(-c2ccccc2F)cc1)C(F)(F)F)CC[C@H](N)C(=O)O ; N=[S@](=O)(CCC(c1ccc(-c2ccccc2Cl)cc1)C(F)(F)F)CC[C@H](N)C(=O)O ; N=[S@@](=O)(CCC(c1ccc(-c2ccccc2Cl)cc1)C(F)(F)F)CC[C@H](N)C(=O)O ; N=S(=O)(CCC(c1ccc(-c2cc(F)cc(F)c2)cc1)C(F)(F)F)CC[C@H](N)C(=O)O]

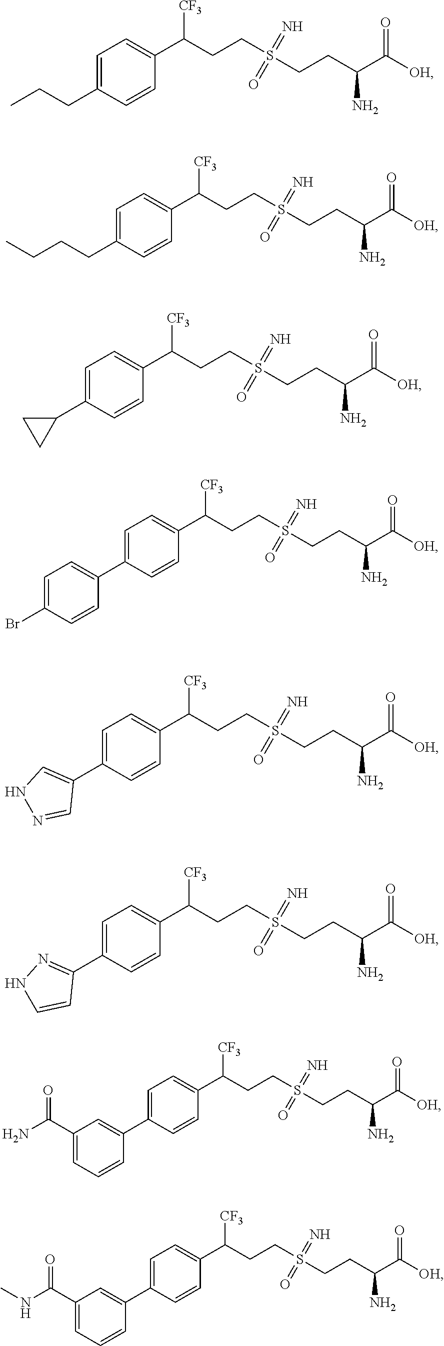

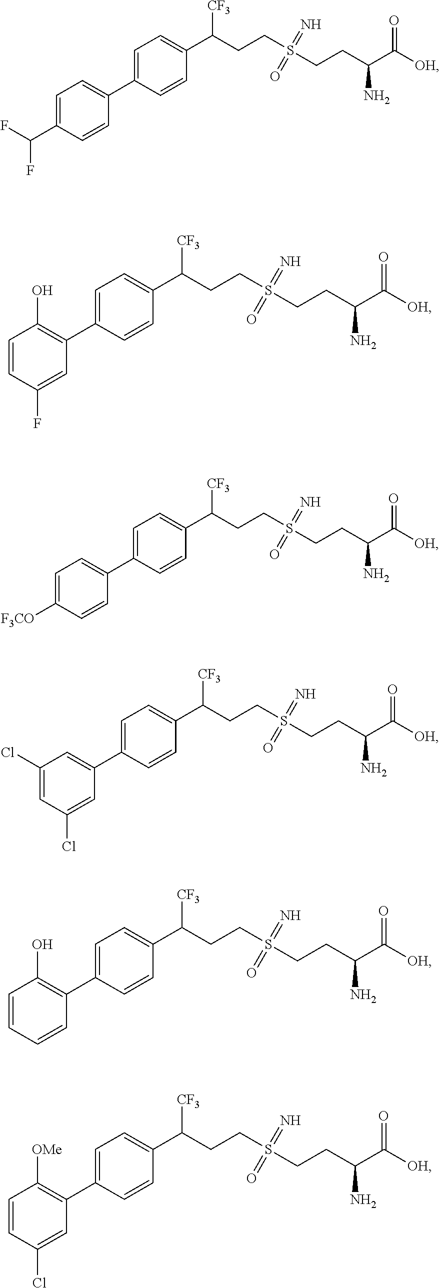 [SMILES: N=S(=O)(CCC(c1ccc(-c2ccc(C(F)F)cc2)cc1)C(F)(F)F)CC[C@H](N)C(=O)O ; N=S(=O)(CCC(c1ccc(-c2cc(F)ccc2O)cc1)C(F)(F)F)CC[C@H](N)C(=O)O ; N=S(=O)(CCC(c1ccc(-c2ccc(OC(F)(F)F)cc2)cc1)C(F)(F)F)CC[C@H](N)C(=O)O ; N=S(=O)(CCC(c1ccc(-c2cc(Cl)cc(Cl)c2)cc1)C(F)(F)F)CC[C@H](N)C(=O)O ; N=S(=O)(CCC(c1ccc(-c2ccccc2O)cc1)C(F)(F)F)CC[C@H](N)C(=O)O ; COc1ccc(Cl)cc1-c1ccc(C(CCS(=N)(=O)CC[C@H](N)C(=O)O)C(F)(F)F)cc1]

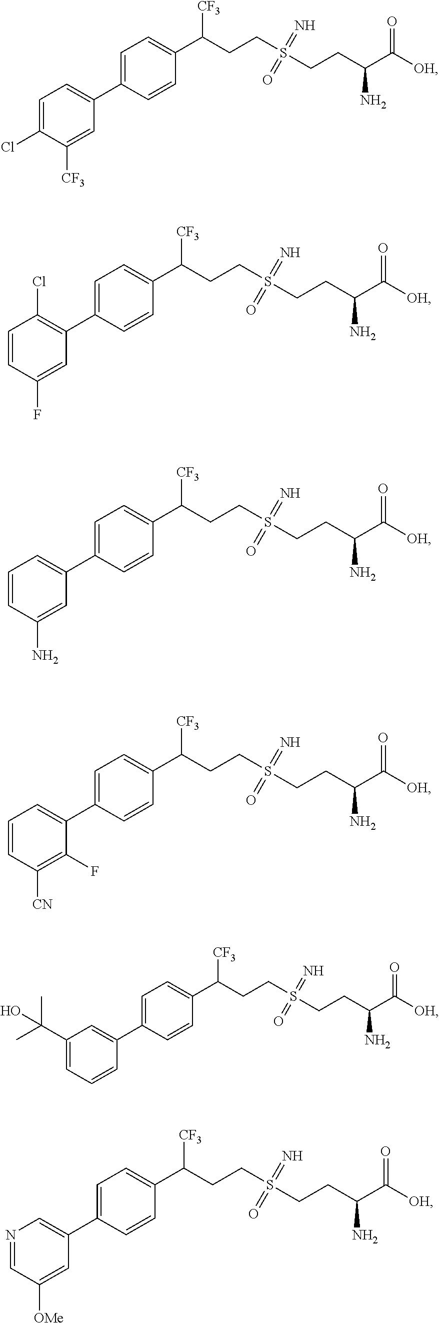 [SMILES: N=S(=O)(CCC(c1ccc(-c2ccc(Cl)c(C(F)(F)F)c2)cc1)C(F)(F)F)CC[C@H](N)C(=O)O ; N=S(=O)(CCC(c1ccc(-c2cc(F)ccc2Cl)cc1)C(F)(F)F)CC[C@H](N)C(=O)O ; N=S(=O)(CCC(c1ccc(-c2cccc(N)c2)cc1)C(F)(F)F)CC[C@H](N)C(=O)O ; N#Cc1cccc(-c2ccc(C(CCS(=N)(=O)CC[C@H](N)C(=O)O)C(F)(F)F)cc2)c1F ; CC(C)(O)c1cccc(-c2ccc(C(CCS(=N)(=O)CC[C@H](N)C(=O)O)C(F)(F)F)cc2)c1 ; COc1cncc(-c2ccc(C(CCS(=N)(=O)CC[C@H](N)C(=O)O)C(F)(F)F)cc2)c1]

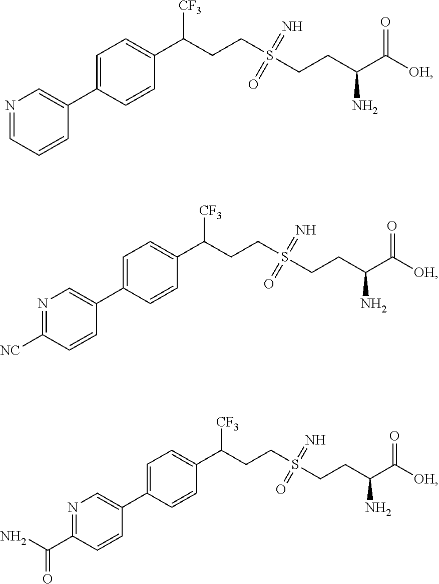 [SMILES: N=S(=O)(CCC(c1ccc(-c2cccnc2)cc1)C(F)(F)F)CC[C@H](N)C(=O)O ; N#Cc1ccc(-c2ccc(C(CCS(=N)(=O)CC[C@H](N)C(=O)O)C(F)(F)F)cc2)cn1 ; N=S(=O)(CCC(c1ccc(-c2ccc(C(N)=O)nc2)cc1)C(F)(F)F)CC[C@H](N)C(=O)O]

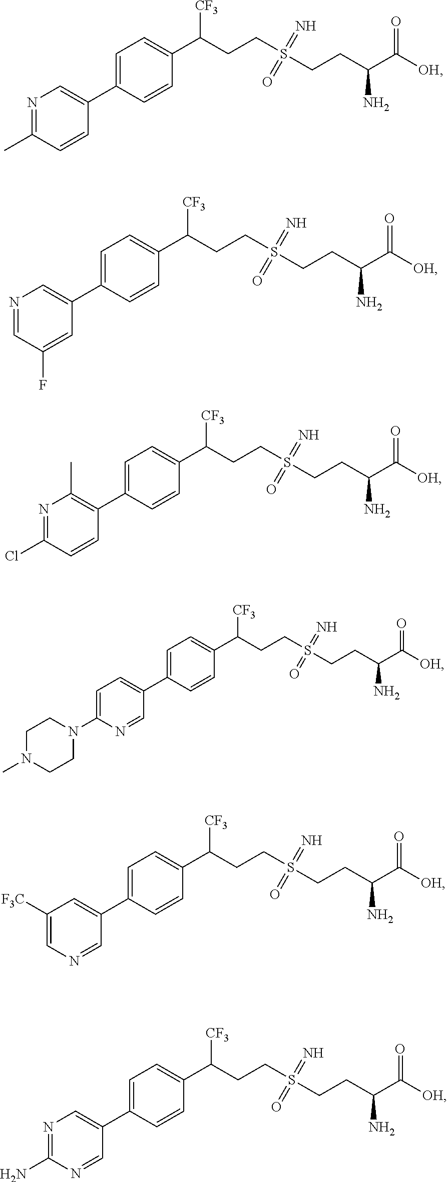 [SMILES: Cc1ccc(-c2ccc(C(CCS(=N)(=O)CC[C@H](N)C(=O)O)C(F)(F)F)cc2)cn1 ; N=S(=O)(CCC(c1ccc(-c2cncc(F)c2)cc1)C(F)(F)F)CC[C@H](N)C(=O)O ; Cc1nc(Cl)ccc1-c1ccc(C(CCS(=N)(=O)CC[C@H](N)C(=O)O)C(F)(F)F)cc1 ; CN1CCN(c2ccc(-c3ccc(C(CCS(=N)(=O)CC[C@H](N)C(=O)O)C(F)(F)F)cc3)cn2)CC1 ; N=S(=O)(CCC(c1ccc(-c2cncc(C(F)(F)F)c2)cc1)C(F)(F)F)CC[C@H](N)C(=O)O ; N=S(=O)(CCC(c1ccc(-c2cnc(N)nc2)cc1)C(F)(F)F)CC[C@H](N)C(=O)O]

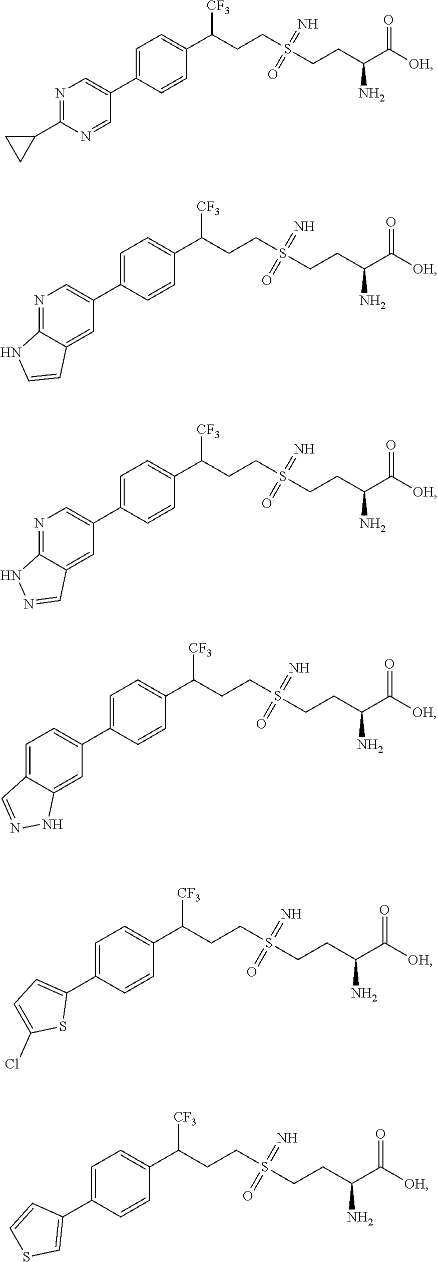 [SMILES: N=S(=O)(CCC(c1ccc(-c2cnc(C3CC3)nc2)cc1)C(F)(F)F)CC[C@H](N)C(=O)O ; N=S(=O)(CCC(c1ccc(-c2cnc3[nH]ccc3c2)cc1)C(F)(F)F)CC[C@H](N)C(=O)O ; N=S(=O)(CCC(c1ccc(-c2cnc3[nH]ncc3c2)cc1)C(F)(F)F)CC[C@H](N)C(=O)O ; N=S(=O)(CCC(c1ccc(-c2ccc3cn[nH]c3c2)cc1)C(F)(F)F)CC[C@H](N)C(=O)O ; N=S(=O)(CCC(c1ccc(-c2ccc(Cl)s2)cc1)C(F)(F)F)CC[C@H](N)C(=O)O ; N=S(=O)(CCC(c1ccc(-c2ccsc2)cc1)C(F)(F)F)CC[C@H](N)C(=O)O]

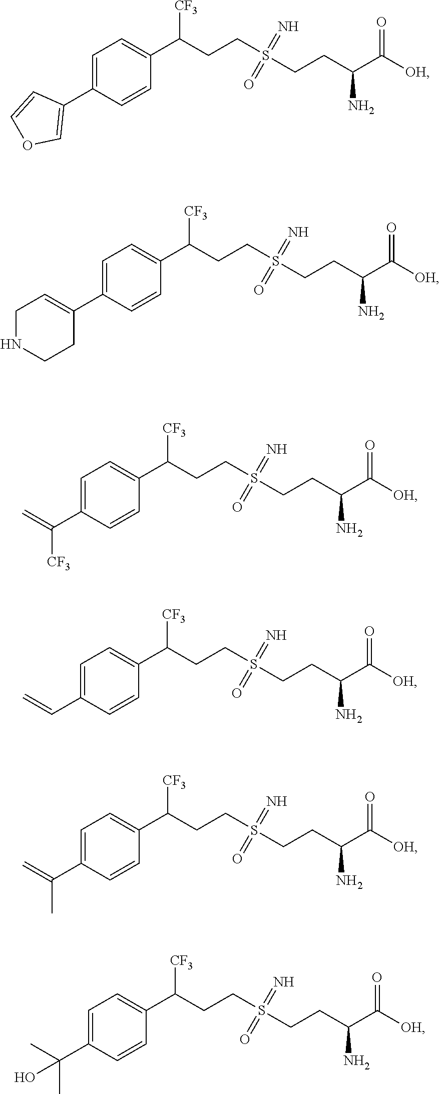 [SMILES: N=S(=O)(CCC(c1ccc(-c2ccoc2)cc1)C(F)(F)F)CC[C@H](N)C(=O)O ; N=S(=O)(CCC(c1ccc(C2=CCNCC2)cc1)C(F)(F)F)CC[C@H](N)C(=O)O ; C=C(c1ccc(C(CCS(=N)(=O)CC[C@H](N)C(=O)O)C(F)(F)F)cc1)C(F)(F)F ; C=Cc1ccc(C(CCS(=N)(=O)CC[C@H](N)C(=O)O)C(F)(F)F)cc1 ; C=C(C)c1ccc(C(CCS(=N)(=O)CC[C@H](N)C(=O)O)C(F)(F)F)cc1 ; CC(C)(O)c1ccc(C(CCS(=N)(=O)CC[C@H](N)C(=O)O)C(F)(F)F)cc1]

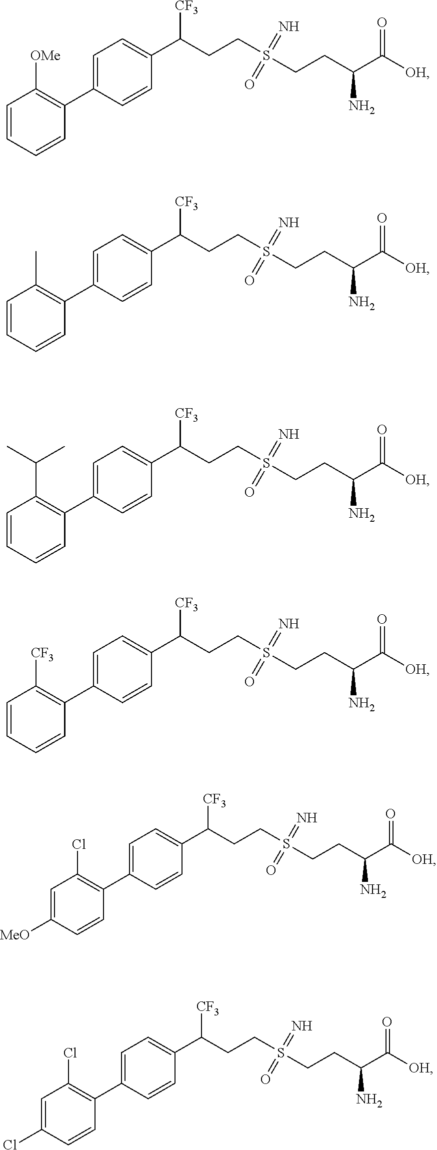 [SMILES: COc1ccccc1-c1ccc(C(CCS(=N)(=O)CC[C@H](N)C(=O)O)C(F)(F)F)cc1 ; Cc1ccccc1-c1ccc(C(CCS(=N)(=O)CC[C@H](N)C(=O)O)C(F)(F)F)cc1 ; CC(C)c1ccccc1-c1ccc(C(CCS(=N)(=O)CC[C@H](N)C(=O)O)C(F)(F)F)cc1 ; N=S(=O)(CCC(c1ccc(-c2ccccc2C(F)(F)F)cc1)C(F)(F)F)CC[C@H](N)C(=O)O ; COc1ccc(-c2ccc(C(CCS(=N)(=O)CC[C@H](N)C(=O)O)C(F)(F)F)cc2)c(Cl)c1 ; N=S(=O)(CCC(c1ccc(-c2ccc(Cl)cc2Cl)cc1)C(F)(F)F)CC[C@H](N)C(=O)O]

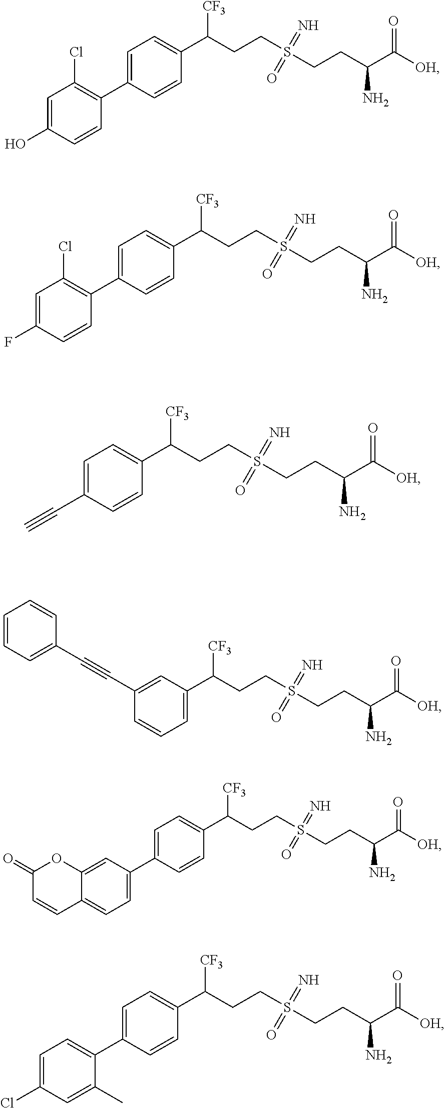 [SMILES: N=S(=O)(CCC(c1ccc(-c2ccc(O)cc2Cl)cc1)C(F)(F)F)CC[C@H](N)C(=O)O ; N=S(=O)(CCC(c1ccc(-c2ccc(F)cc2Cl)cc1)C(F)(F)F)CC[C@H](N)C(=O)O ; C#Cc1ccc(C(CCS(=N)(=O)CC[C@H](N)C(=O)O)C(F)(F)F)cc1 ; N=S(=O)(CCC(c1cccc(C#Cc2ccccc2)c1)C(F)(F)F)CC[C@H](N)C(=O)O ; N=S(=O)(CCC(c1ccc(-c2ccc3ccc(=O)oc3c2)cc1)C(F)(F)F)CC[C@H](N)C(=O)O ; Cc1cc(Cl)ccc1-c1ccc(C(CCS(=N)(=O)CC[C@H](N)C(=O)O)C(F)(F)F)cc1]

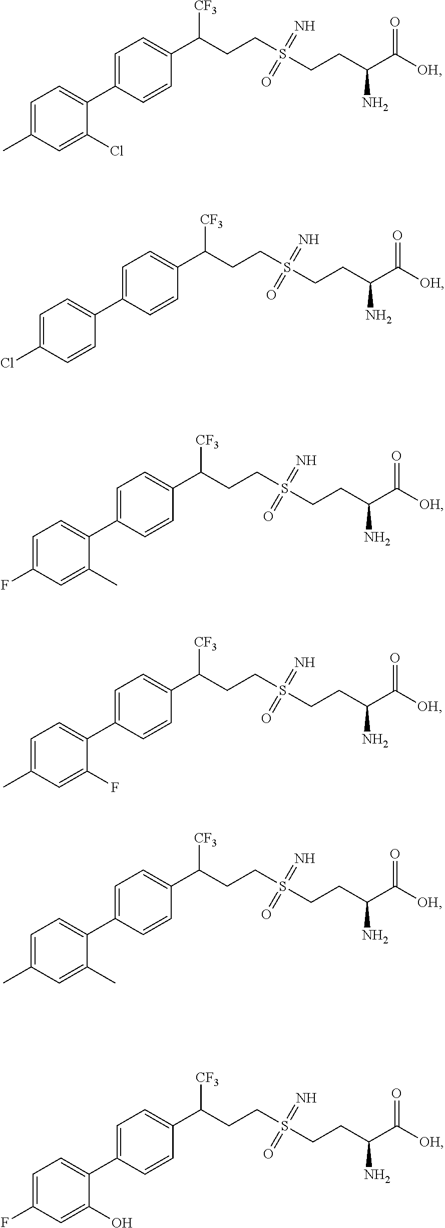 [SMILES: Cc1ccc(-c2ccc(C(CCS(=N)(=O)CC[C@H](N)C(=O)O)C(F)(F)F)cc2)c(Cl)c1 ; N=S(=O)(CCC(c1ccc(-c2ccc(Cl)cc2)cc1)C(F)(F)F)CC[C@H](N)C(=O)O ; Cc1cc(F)ccc1-c1ccc(C(CCS(=N)(=O)CC[C@H](N)C(=O)O)C(F)(F)F)cc1 ; Cc1ccc(-c2ccc(C(CCS(=N)(=O)CC[C@H](N)C(=O)O)C(F)(F)F)cc2)c(F)c1 ; Cc1ccc(-c2ccc(C(CCS(=N)(=O)CC[C@H](N)C(=O)O)C(F)(F)F)cc2)c(C)c1 ; N=S(=O)(CCC(c1ccc(-c2ccc(F)cc2O)cc1)C(F)(F)F)CC[C@H](N)C(=O)O]

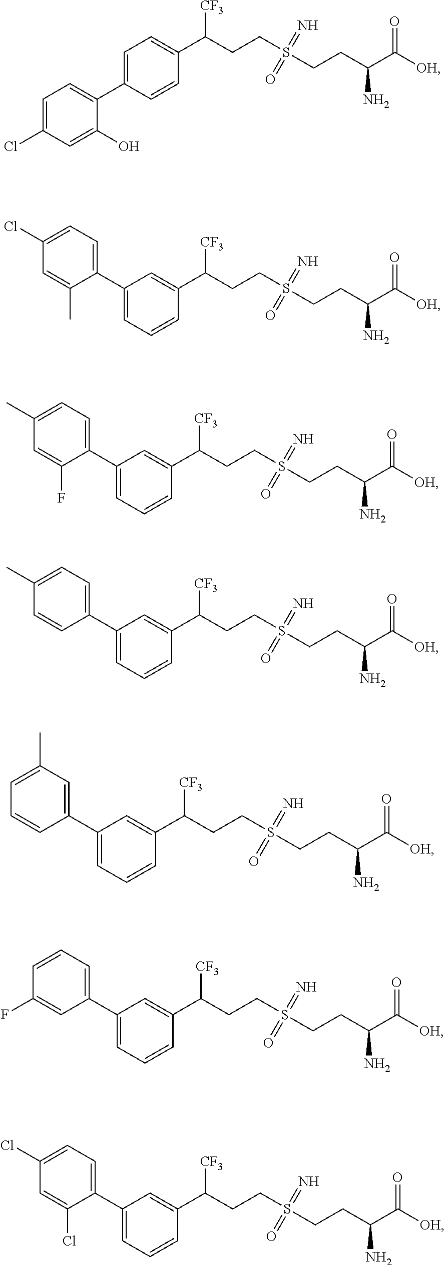

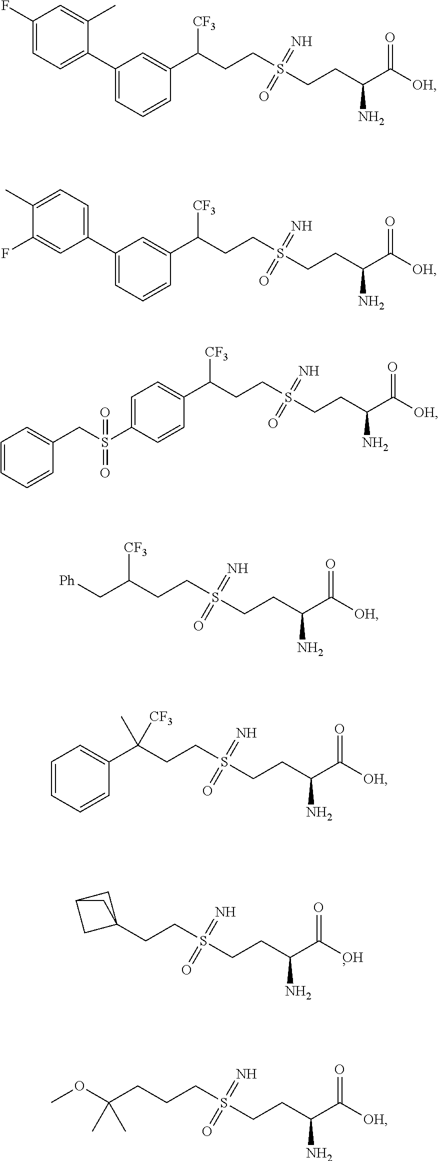

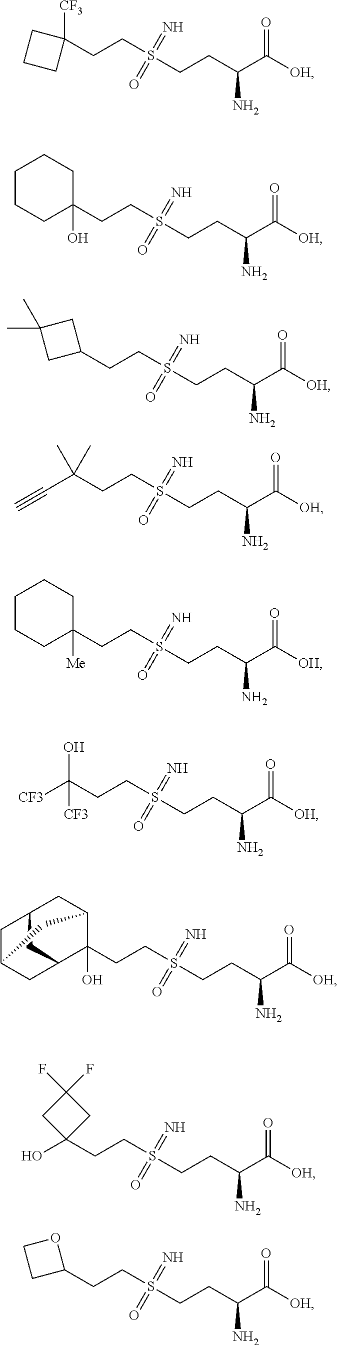

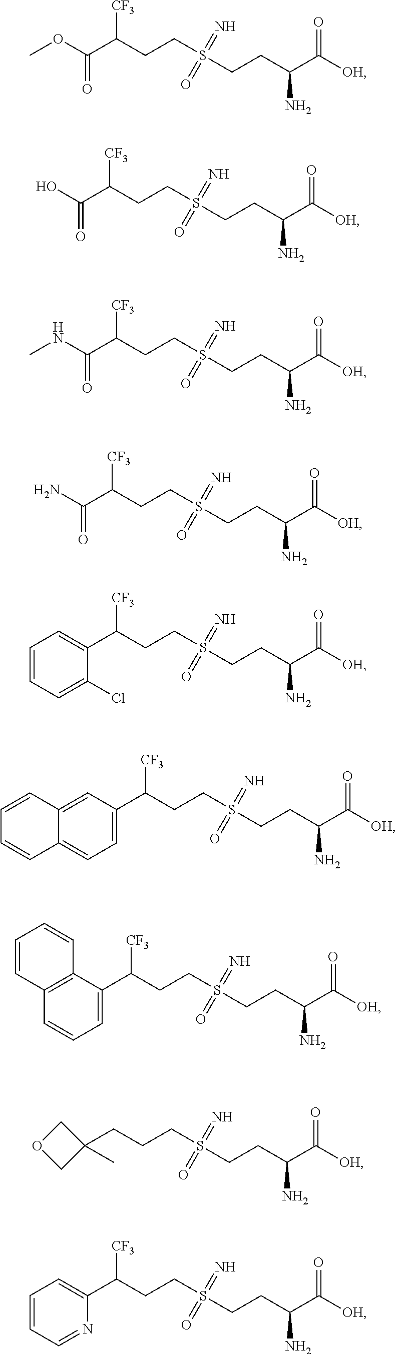

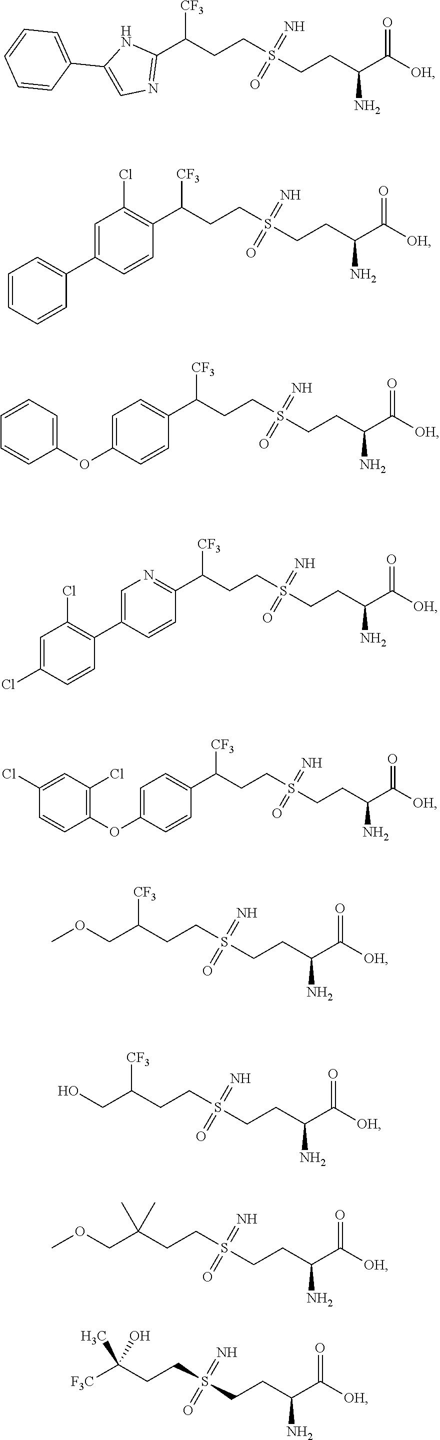

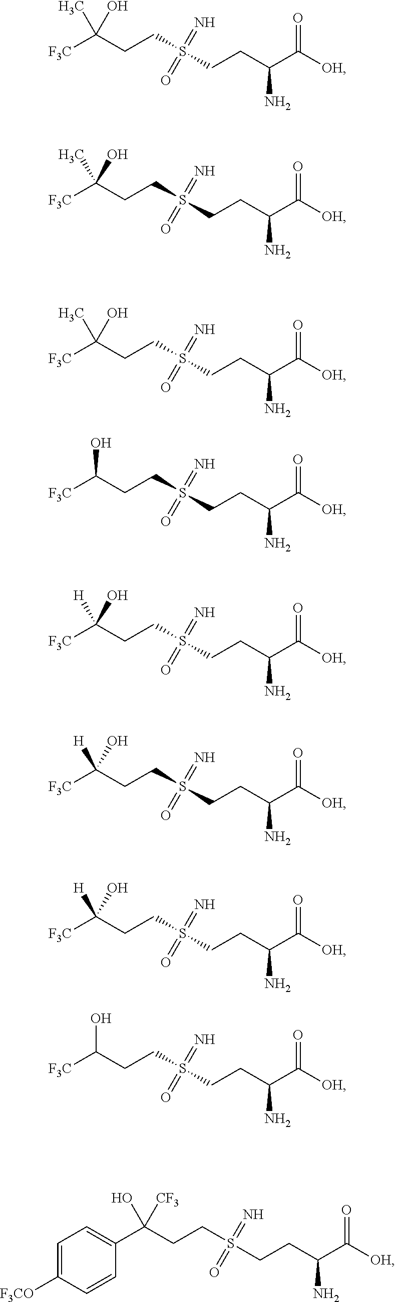

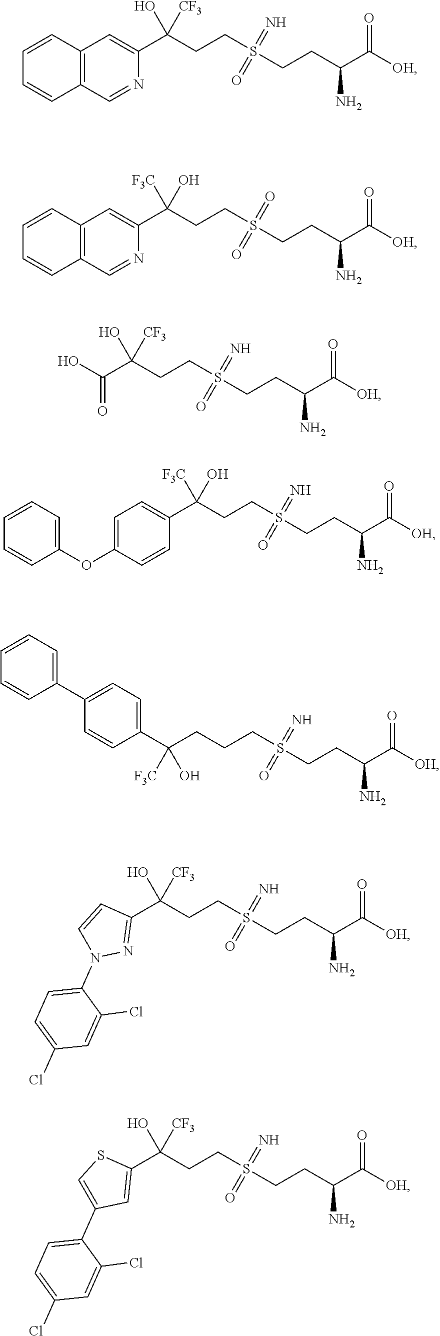

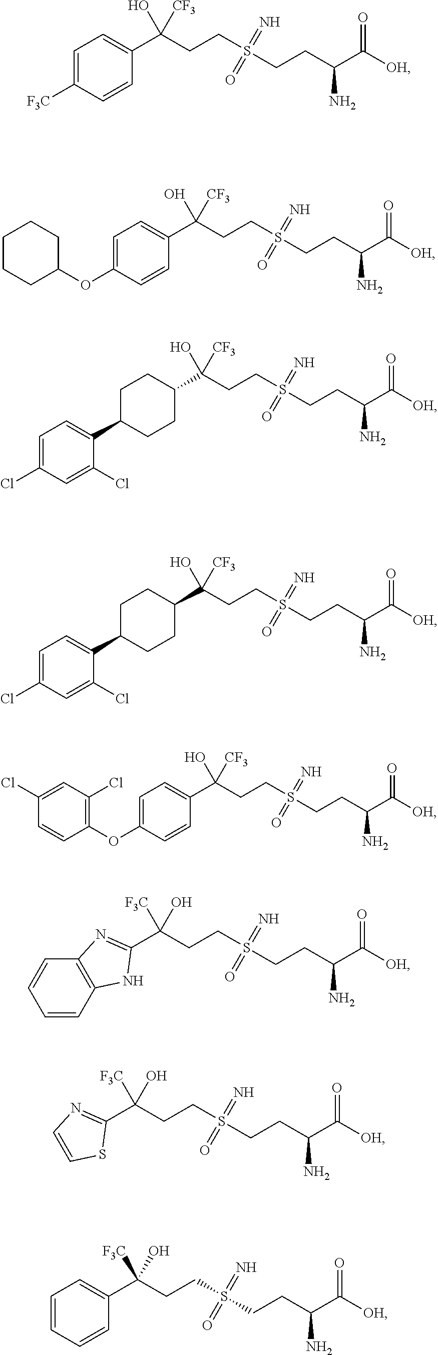

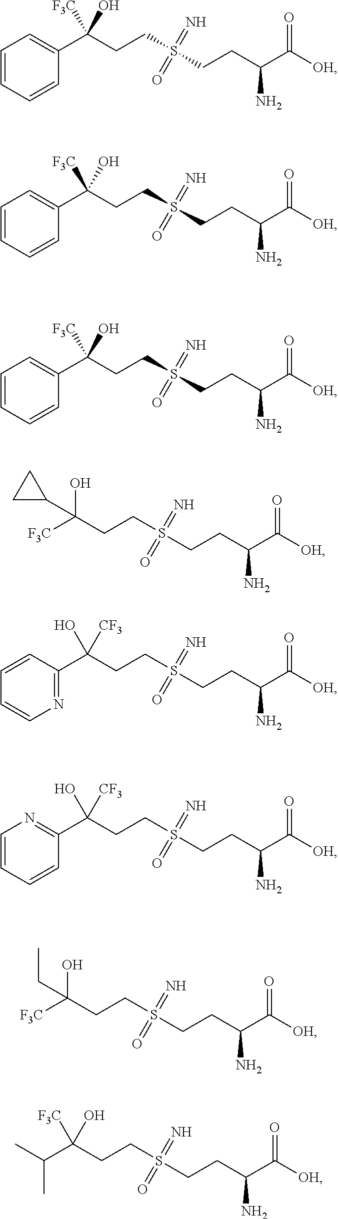

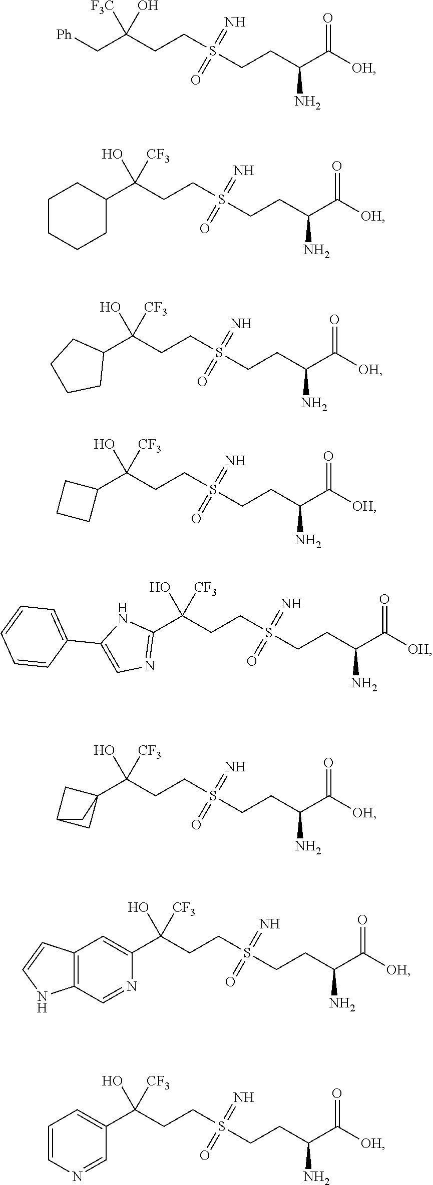

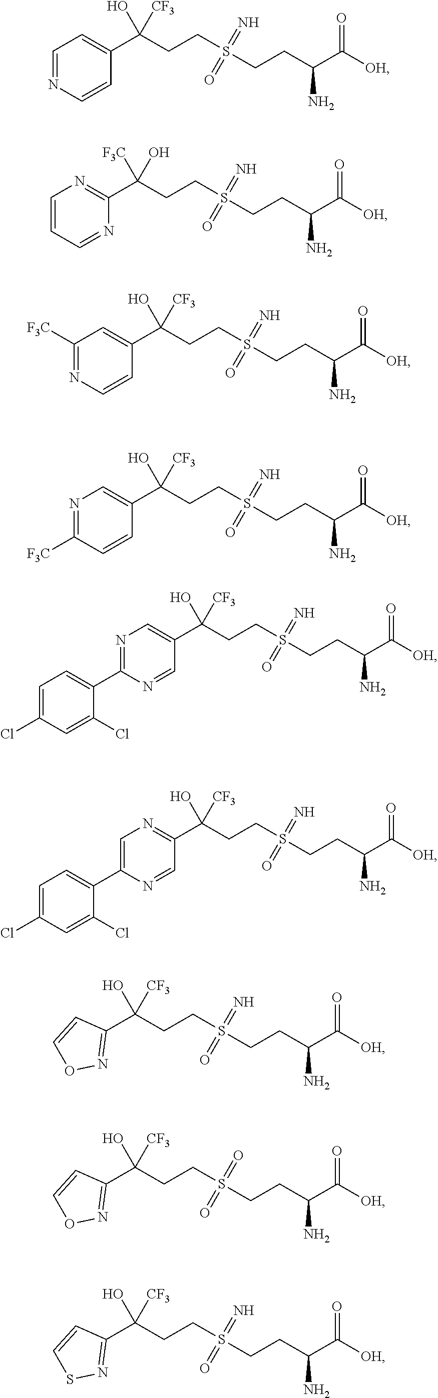

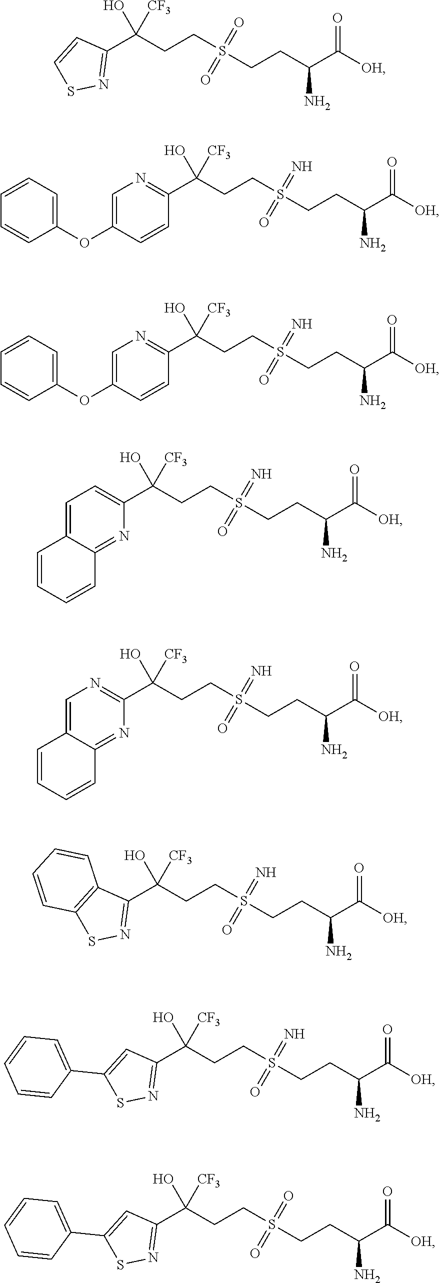

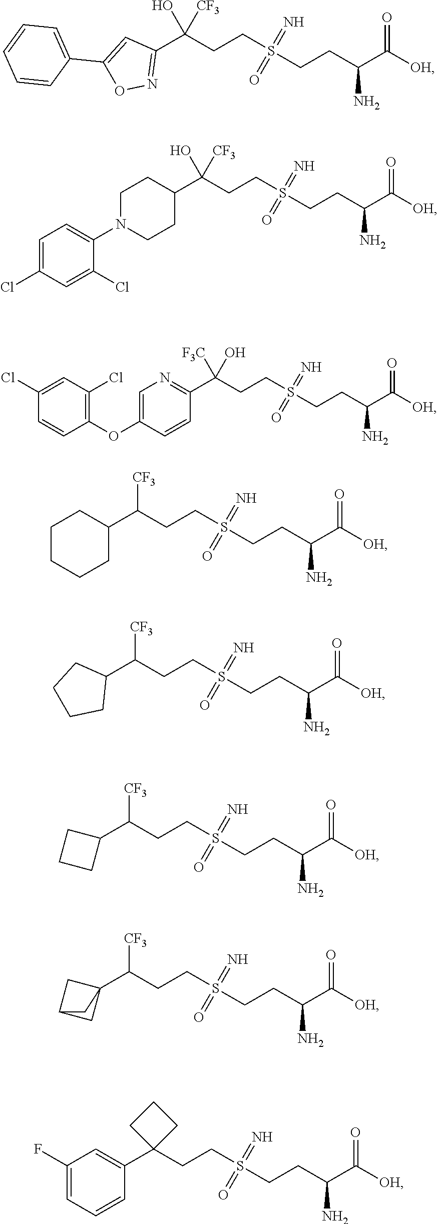

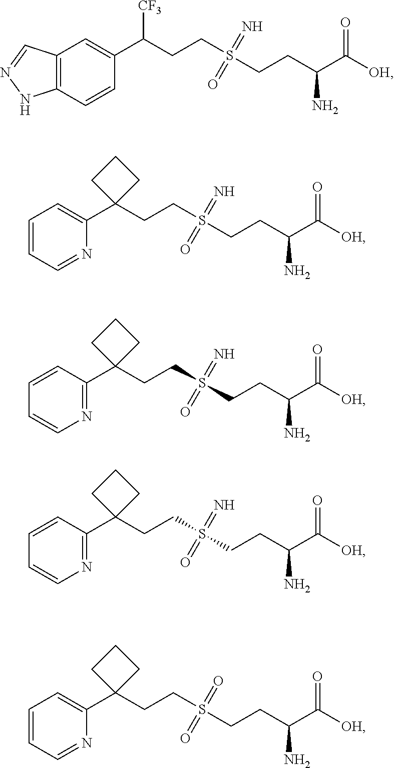 [SMILES: N=S(=O)(CCC(c1ccc2[nH]ncc2c1)C(F)(F)F)CC[C@H](N)C(=O)O ; N=S(=O)(CC[C@H](N)C(=O)O)CCC1(c2ccccn2)CCC1 ; N=[S@@](=O)(CC[C@H](N)C(=O)O)CCC1(c2ccccn2)CCC1 ; N=[S@](=O)(CC[C@H](N)C(=O)O)CCC1(c2ccccn2)CCC1 ; N[C@@H](CCS(=O)(=O)CCC1(c2ccccn2)CCC1)C(=O)O]

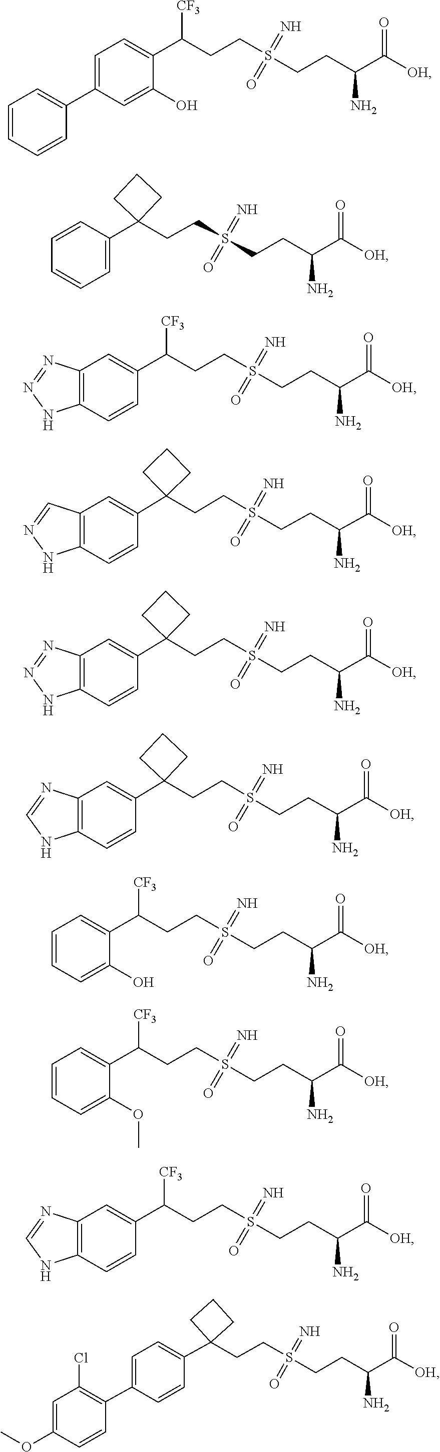

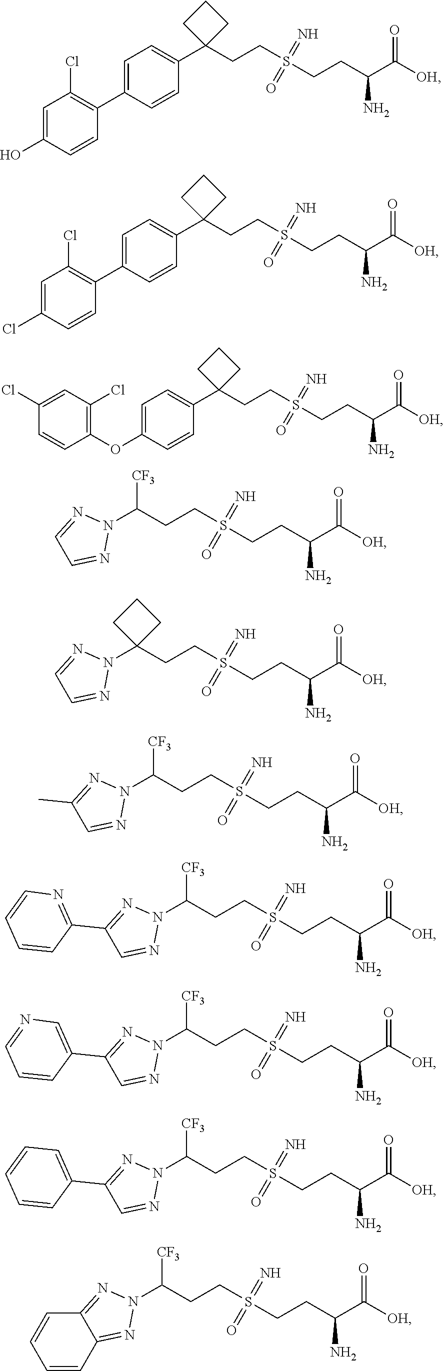

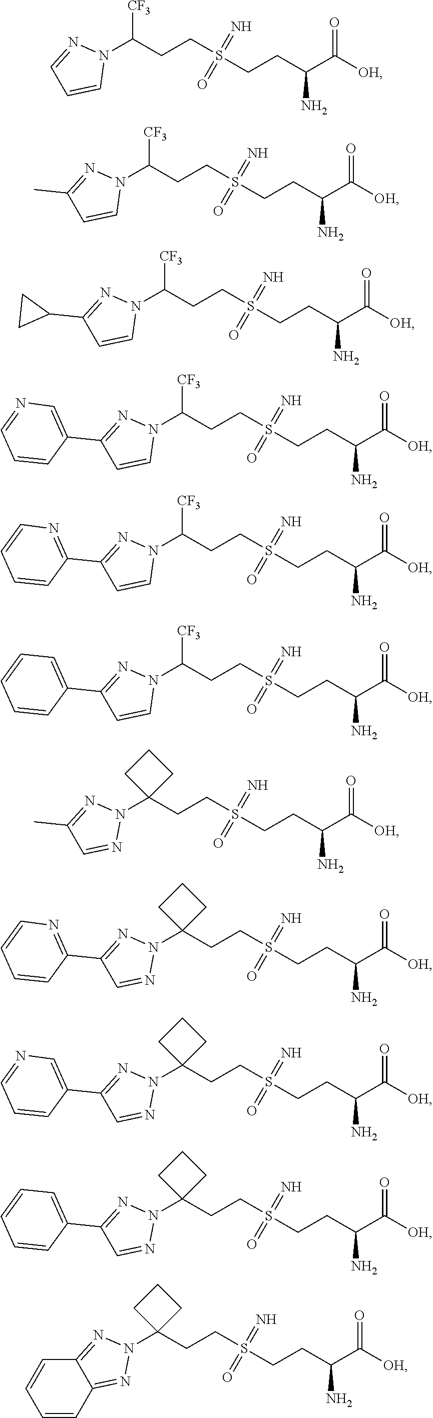

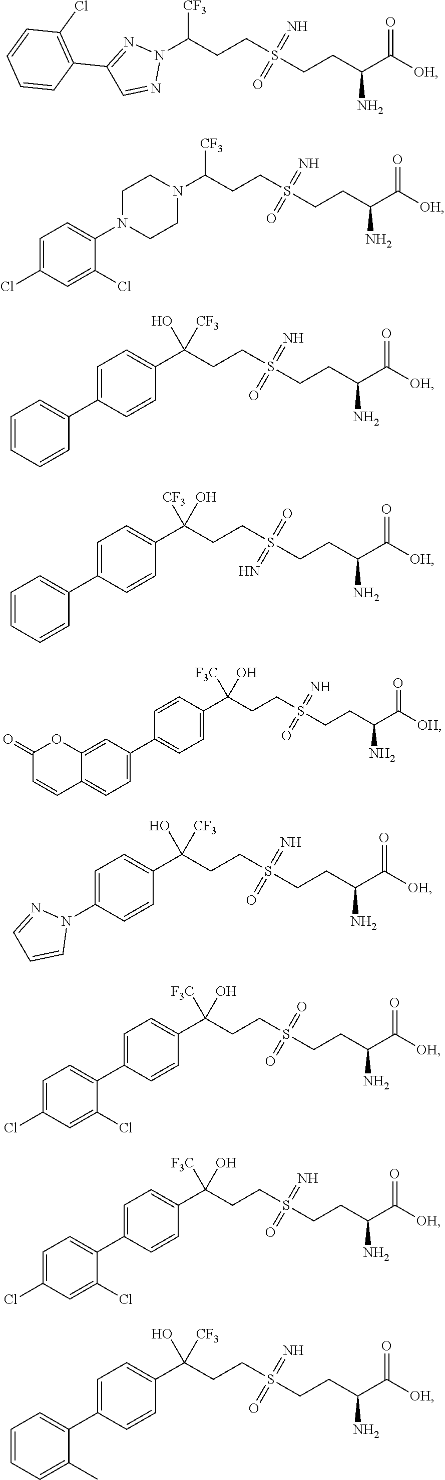

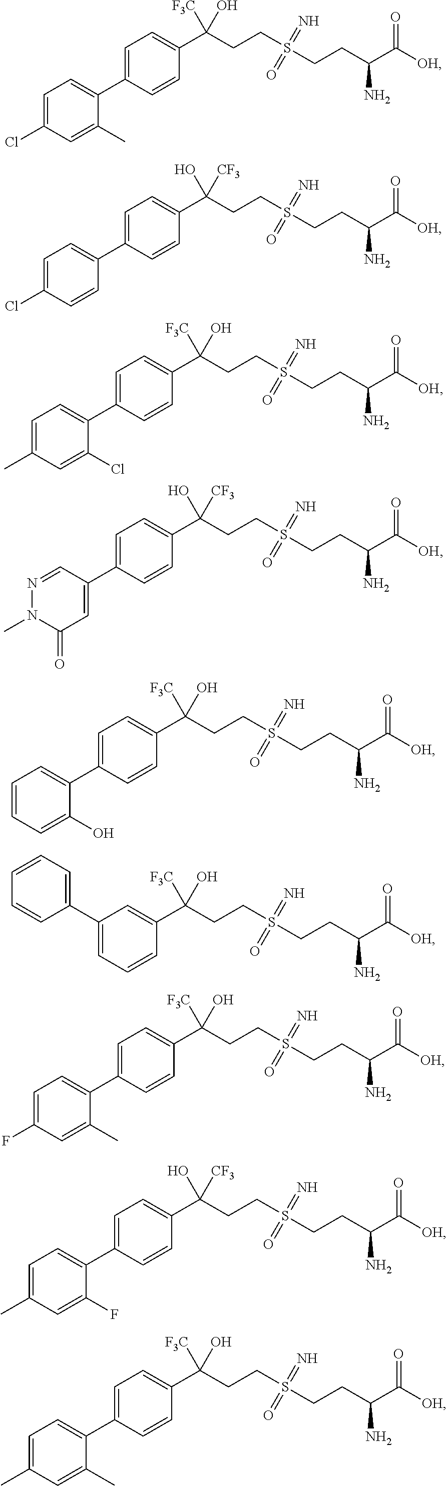

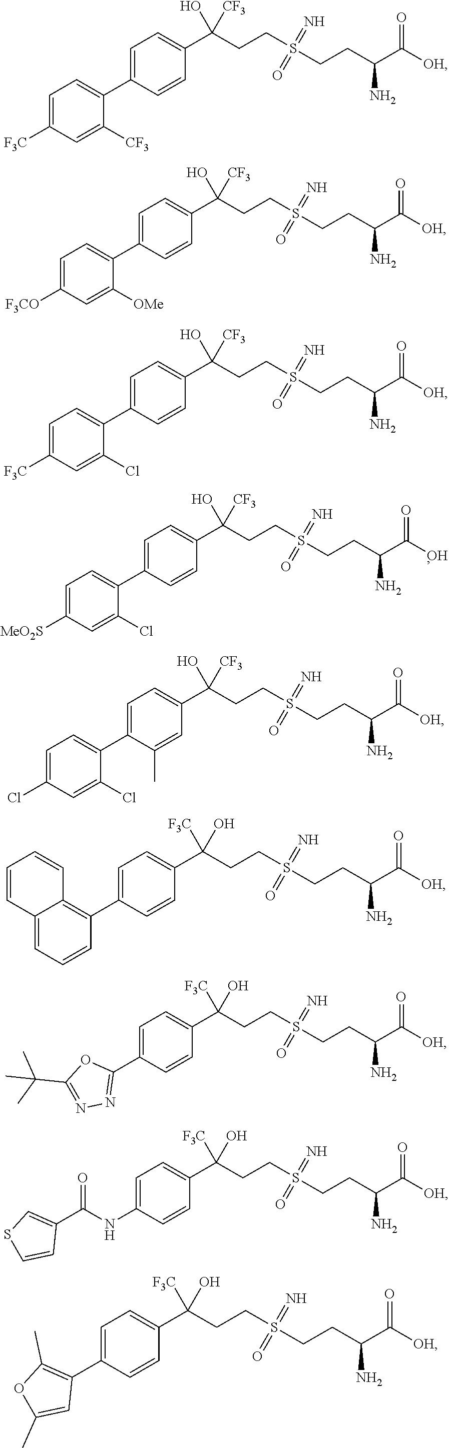

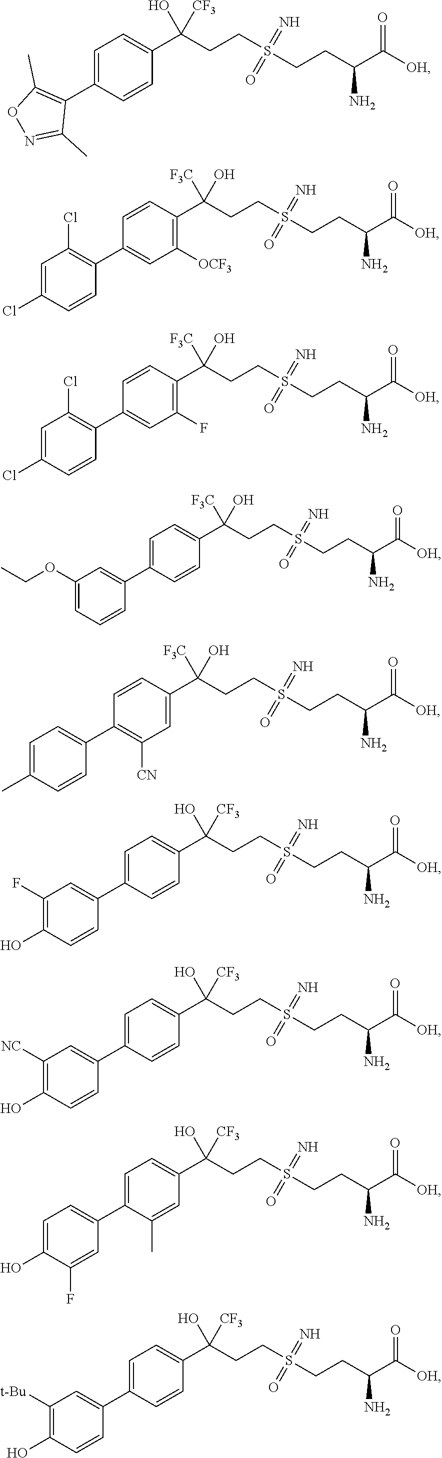

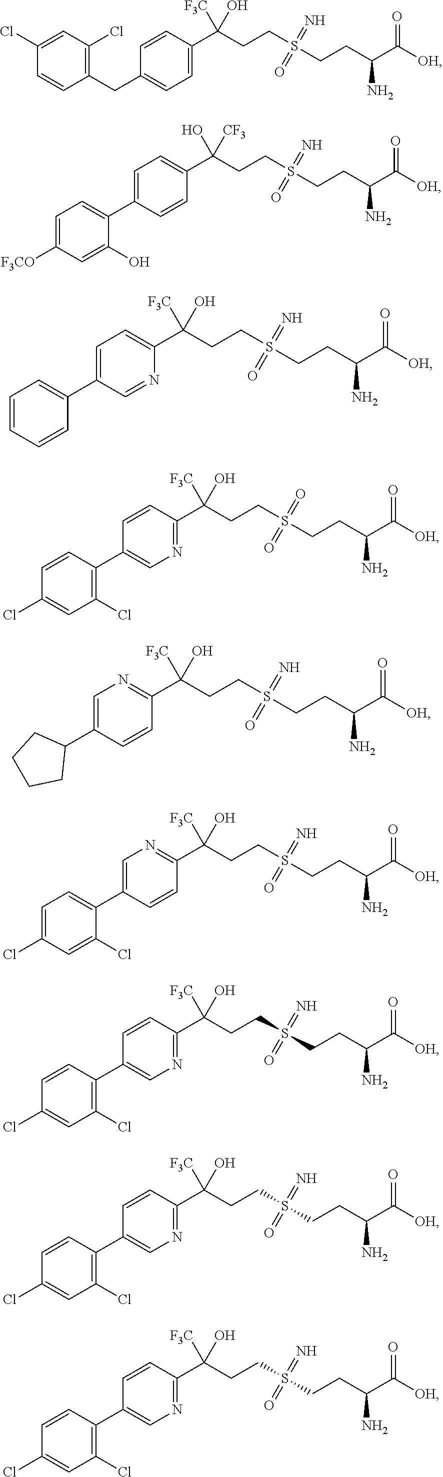

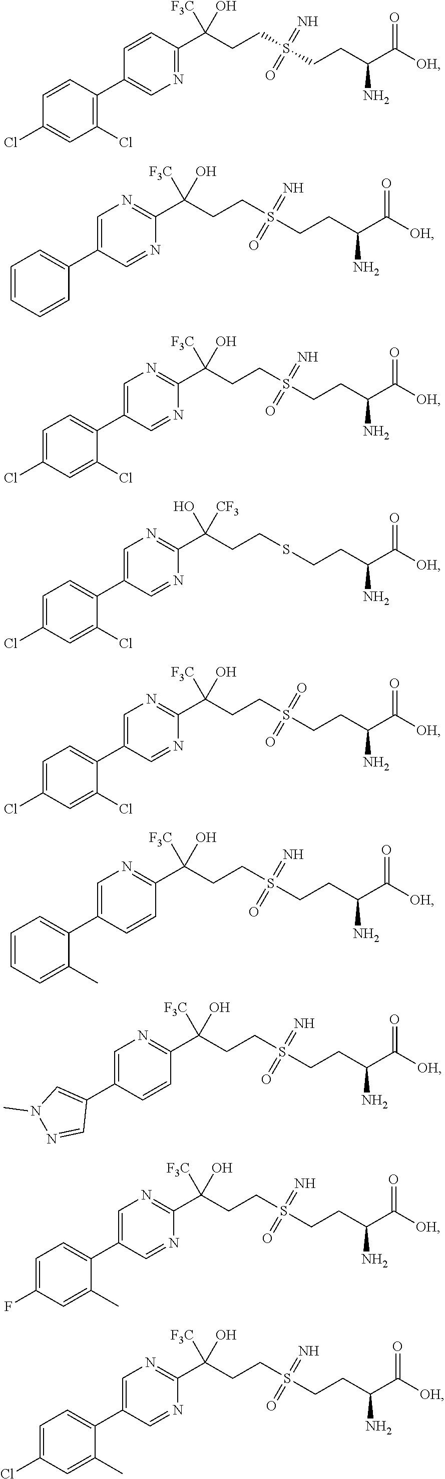

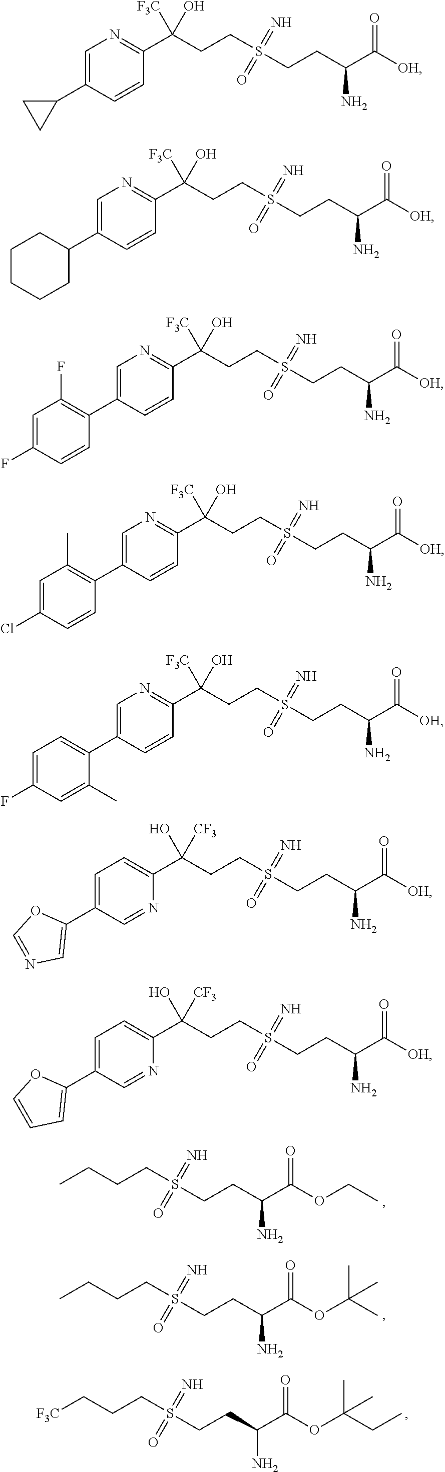

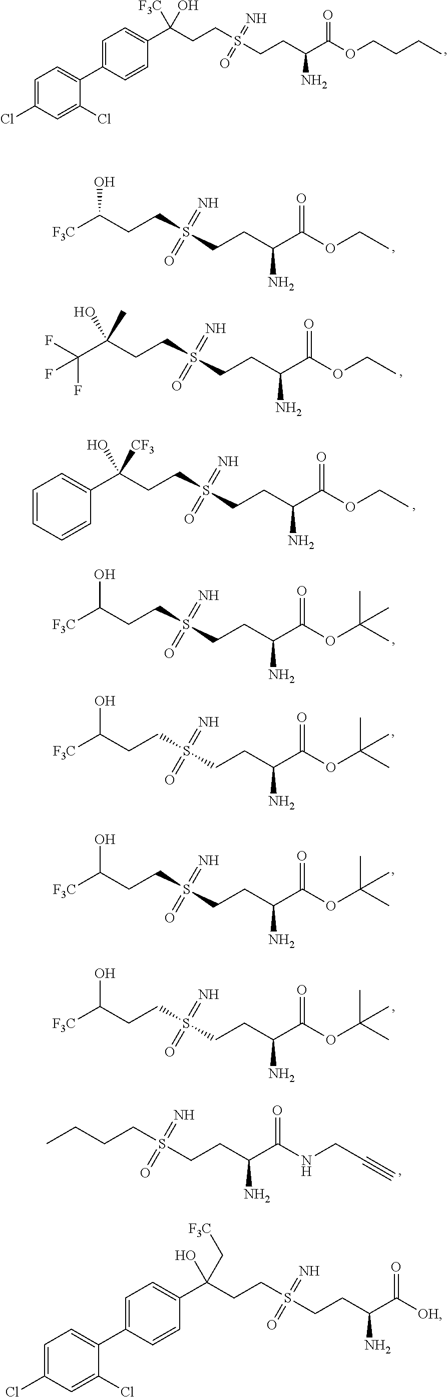

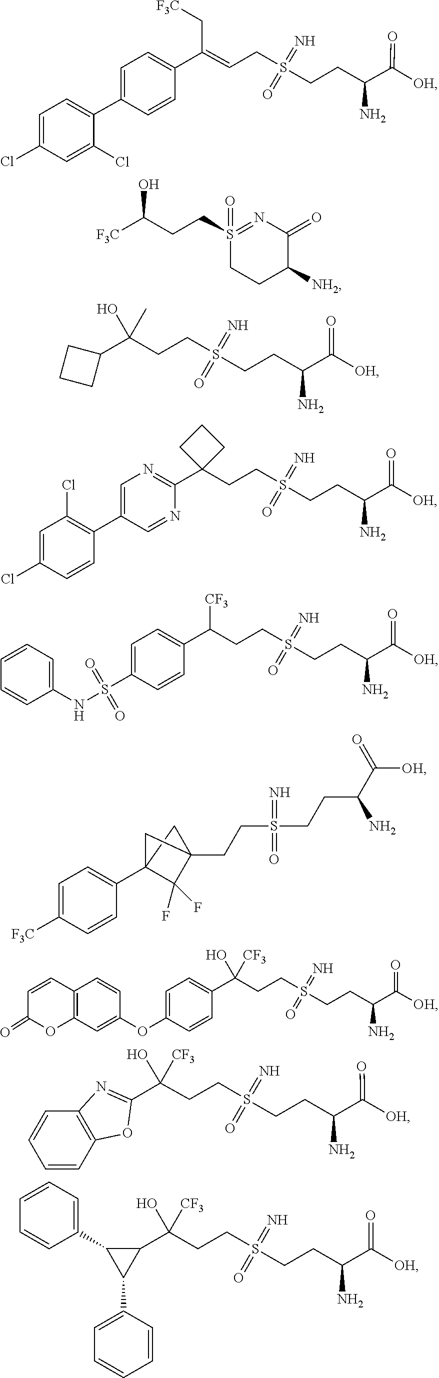

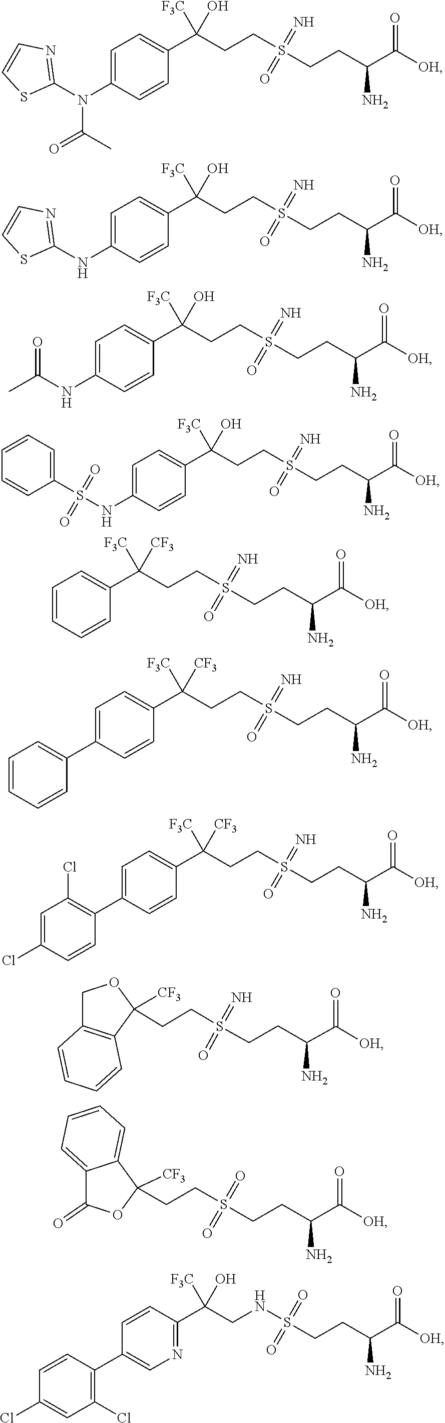

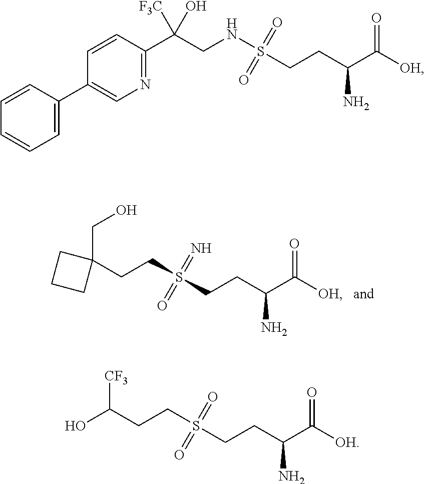 [SMILES: N[C@@H](CCS(=O)(=O)NCC(O)(c1ccc(-c2ccccc2)cn1)C(F)(F)F)C(=O)O ; N=[S@@](=O)(CC[C@H](N)C(=O)O)CCC1(CO)CCC1 ; N[C@@H](CCS(=O)(=O)CCC(O)C(F)(F)F)C(=O)O]

41. A compound, a diastereomer or an enantiomer of the compound, or a pharmaceutically acceptable salt of any of the foregoing, or a deuterated derivative of any of the foregoing; wherein the compound is selected from the group consisting of:

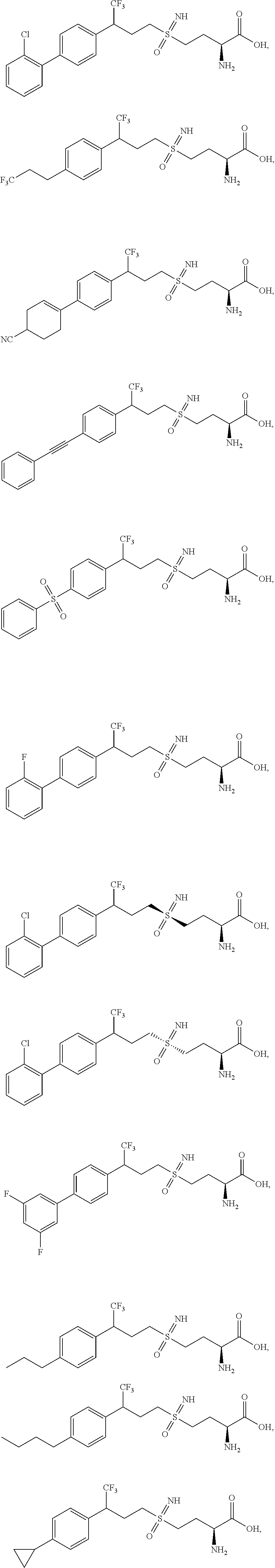

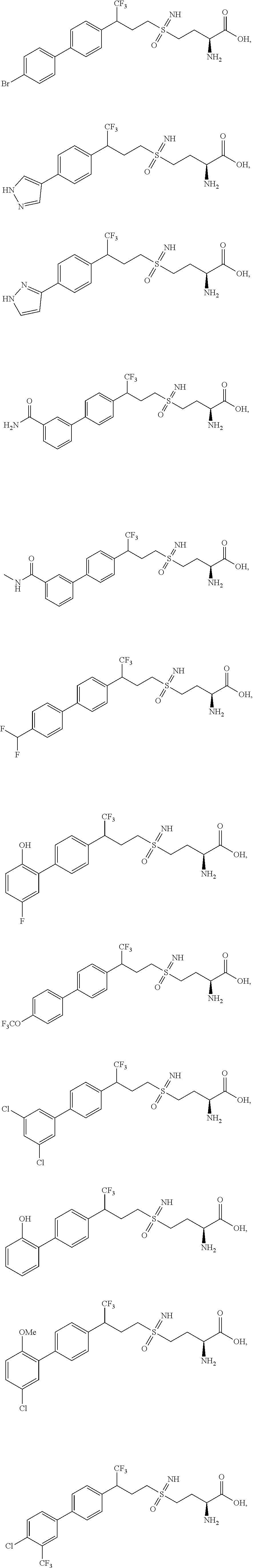

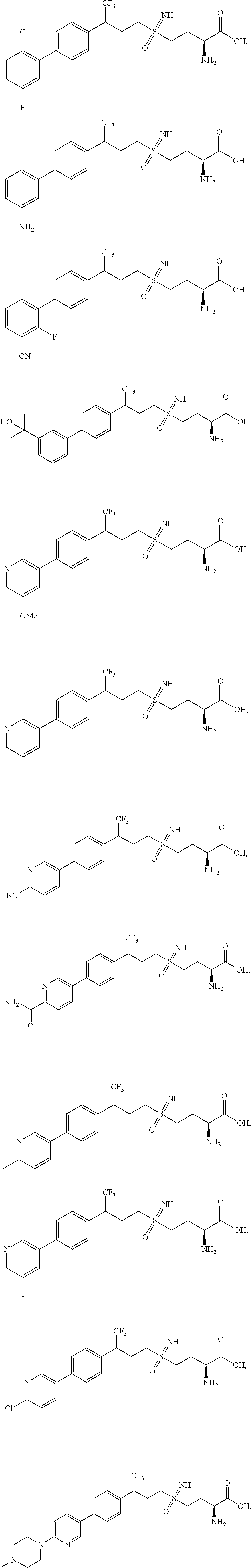

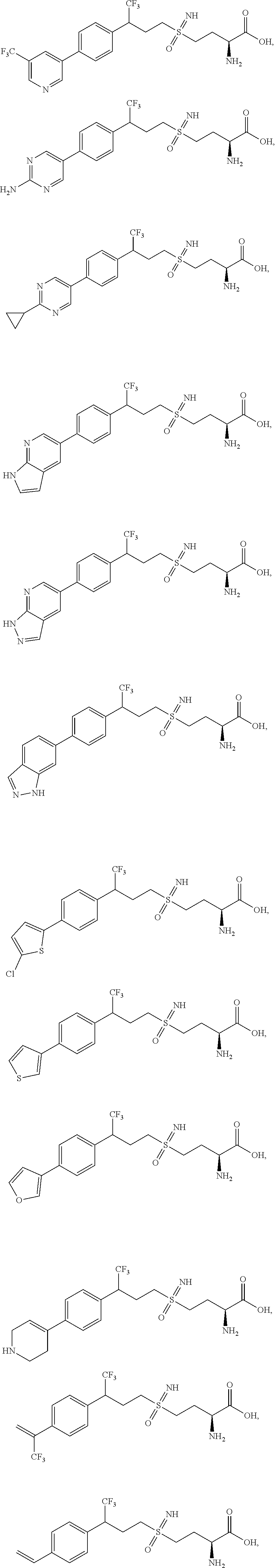

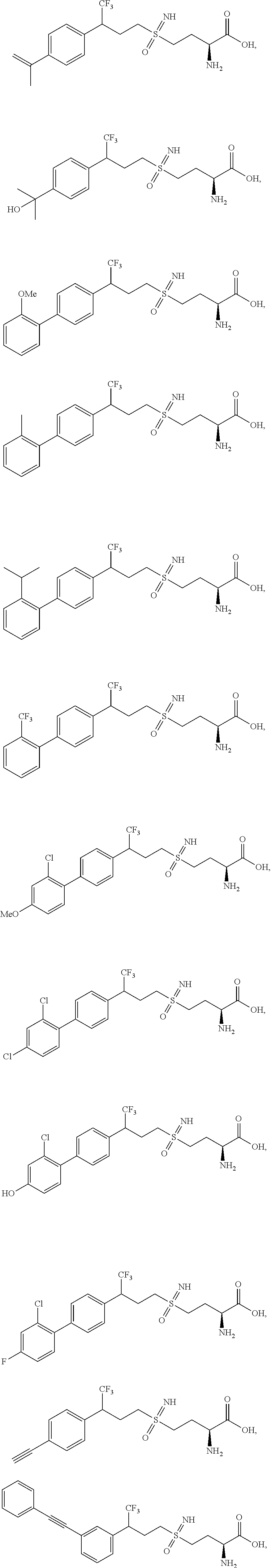

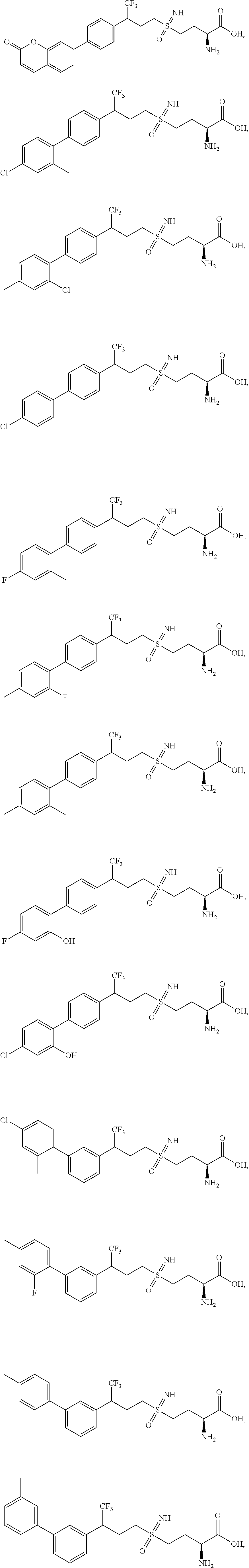

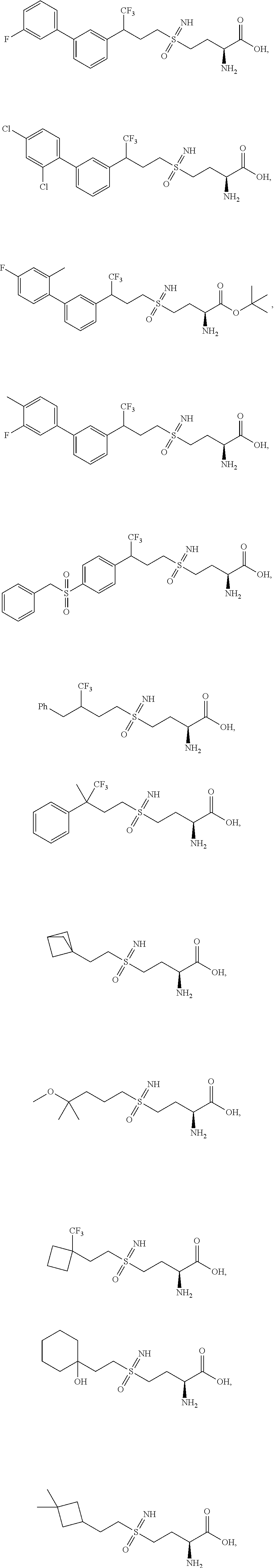

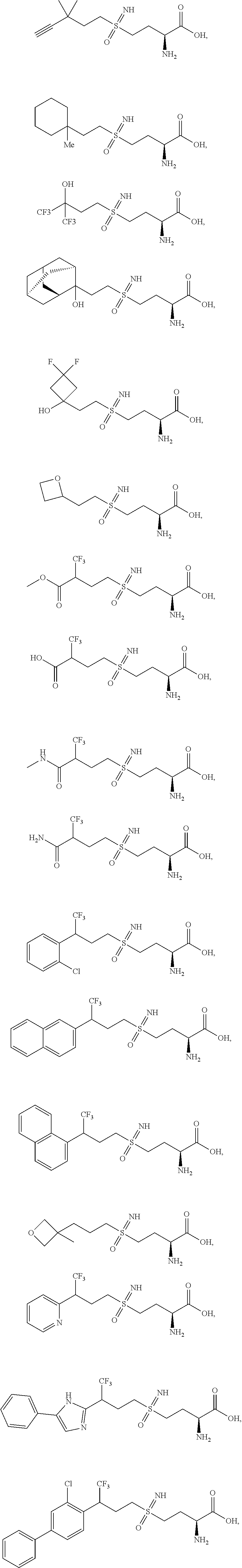

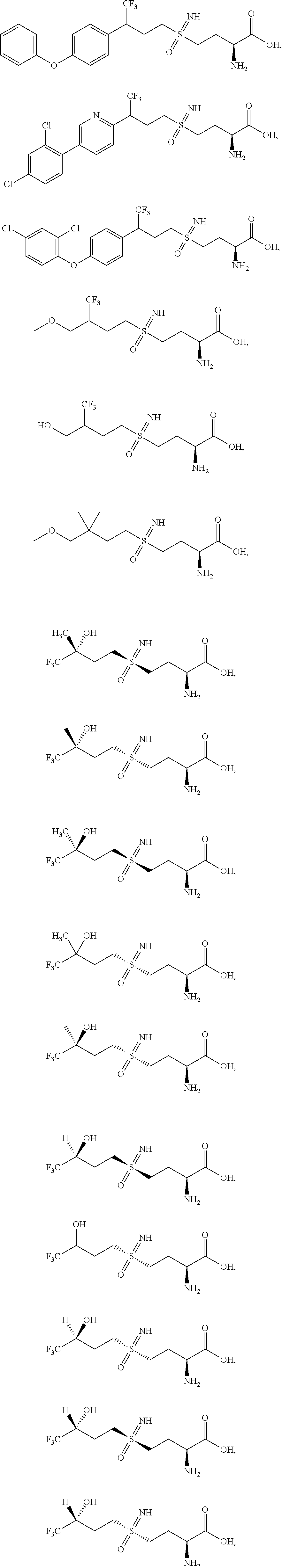

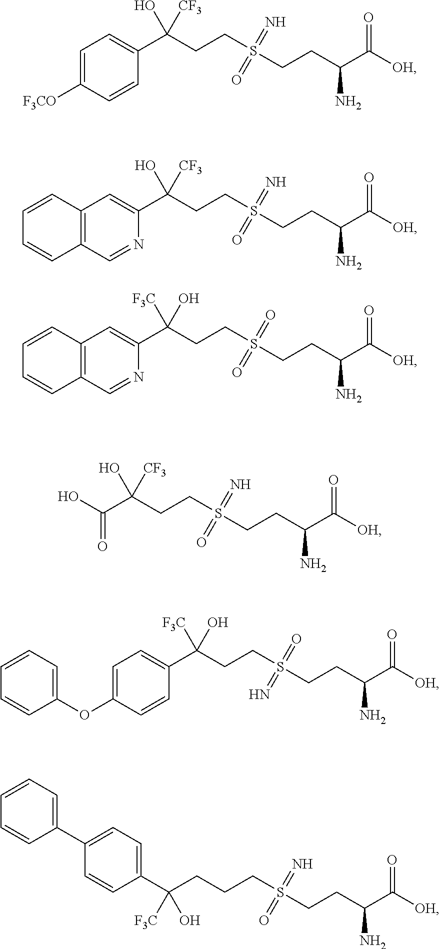 [SMILES: N=S(=O)(CC[C@H](N)C(=O)O)CCC(O)(c1ccc(OC(F)(F)F)cc1)C(F)(F)F ; N=S(=O)(CC[C@H](N)C(=O)O)CCC(O)(c1cc2ccccc2cn1)C(F)(F)F ; N[C@@H](CCS(=O)(=O)CCC(O)(c1cc2ccccc2cn1)C(F)(F)F)C(=O)O ; N=S(=O)(CC[C@H](N)C(=O)O)CCC(O)(C(=O)O)C(F)(F)F ; N=S(=O)(CC[C@H](N)C(=O)O)CCC(O)(c1ccc(Oc2ccccc2)cc1)C(F)(F)F ; N=S(=O)(CCCC(O)(c1ccc(-c2ccccc2)cc1)C(F)(F)F)CC[C@H](N)C(=O)O]

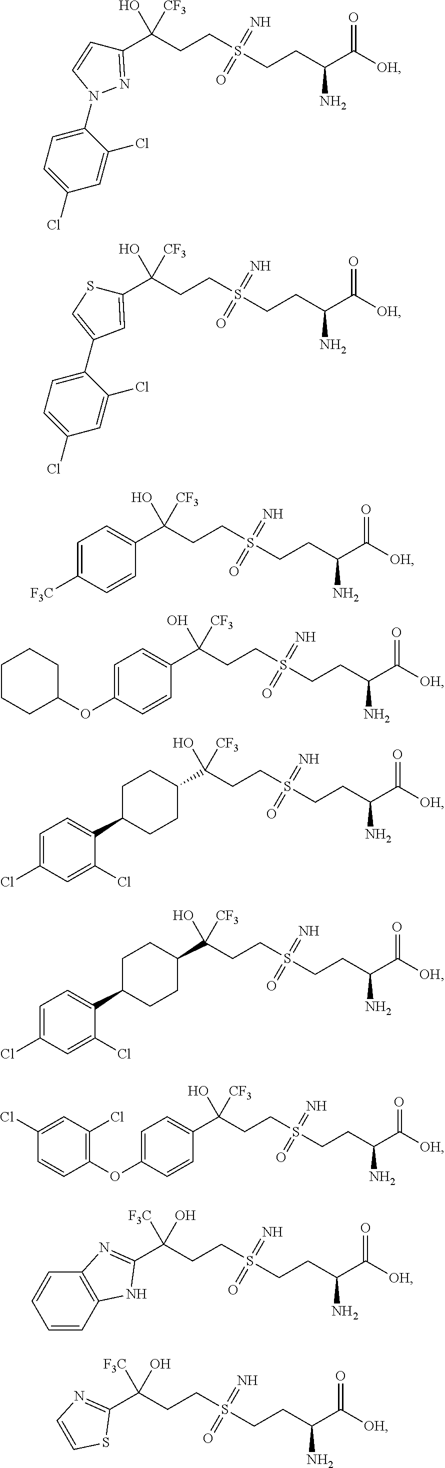

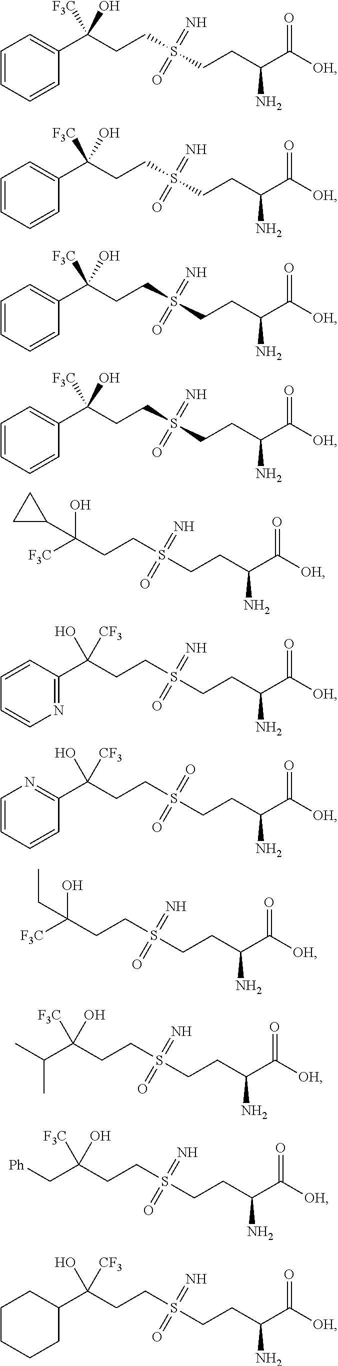

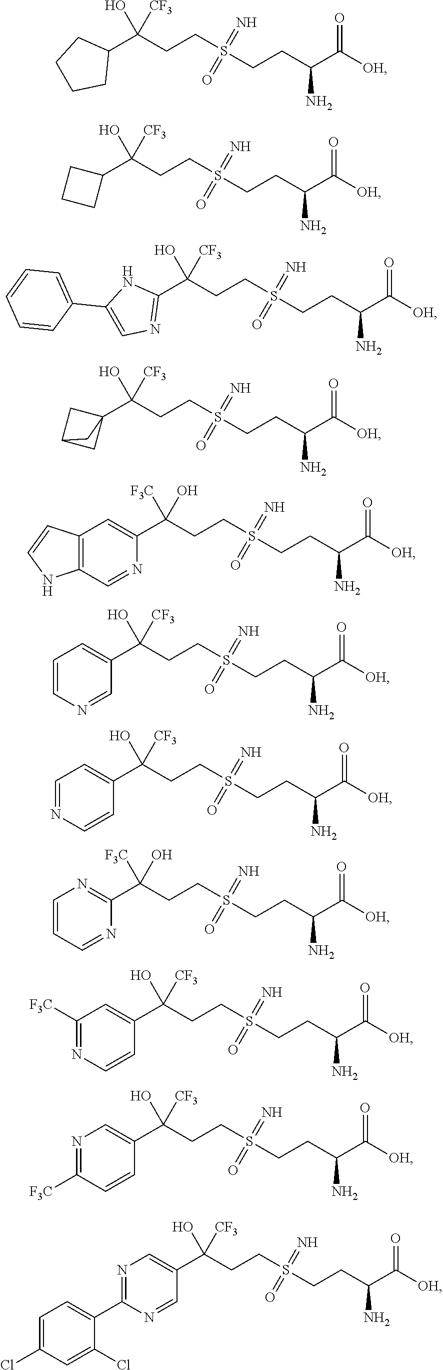

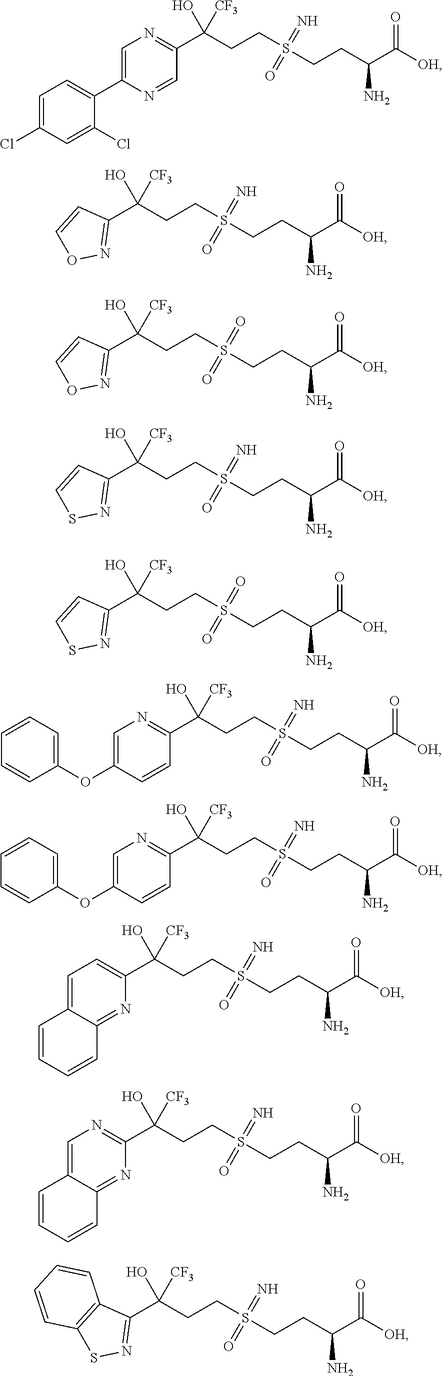

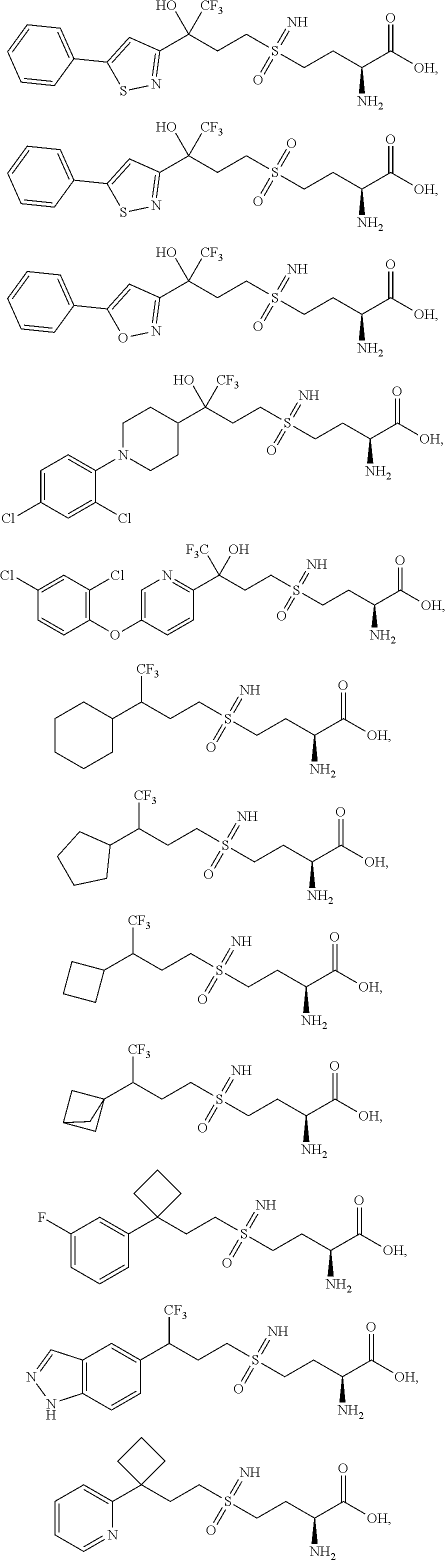

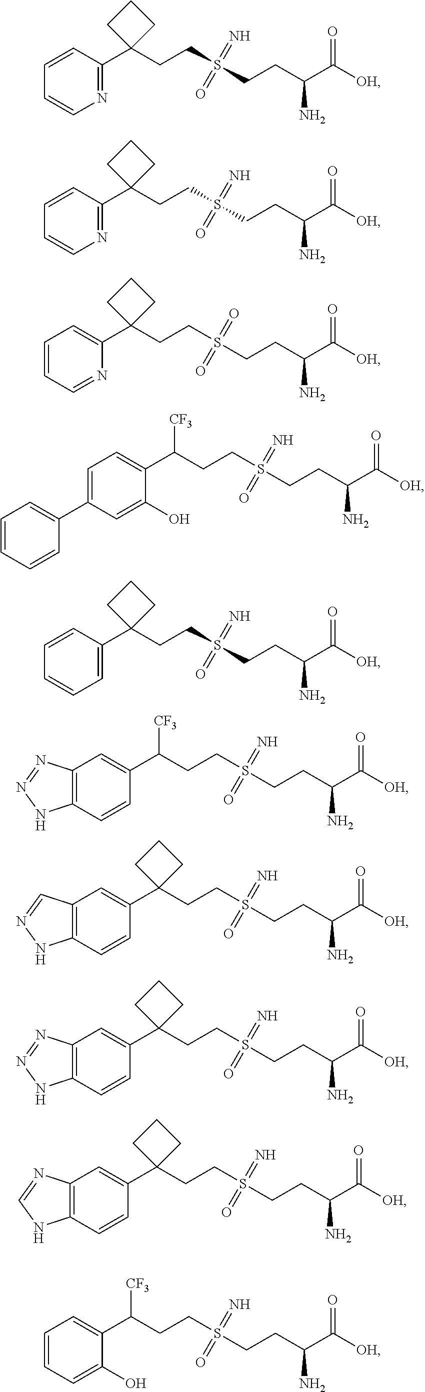

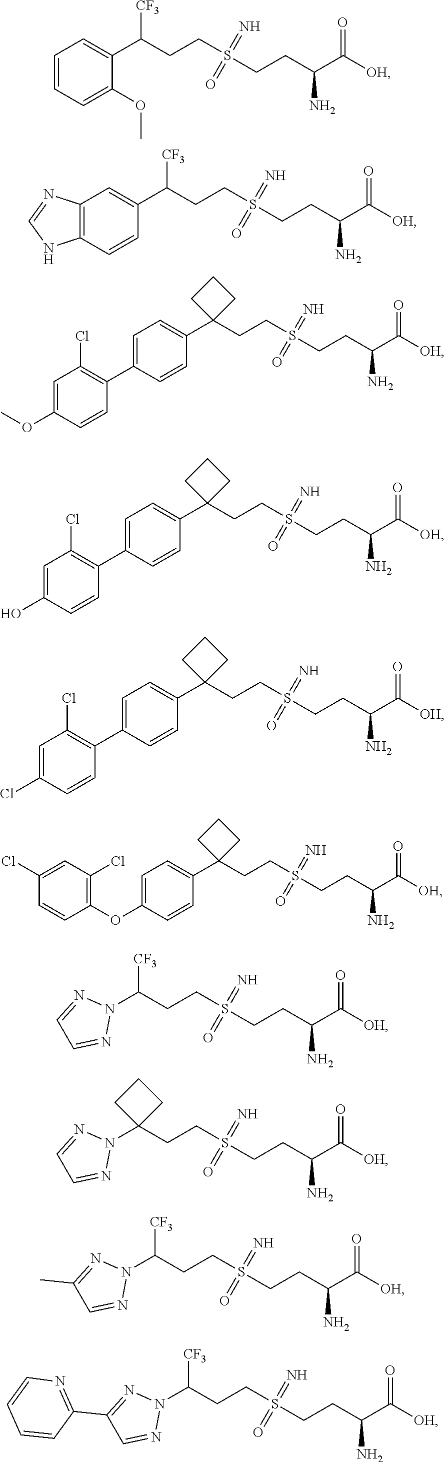

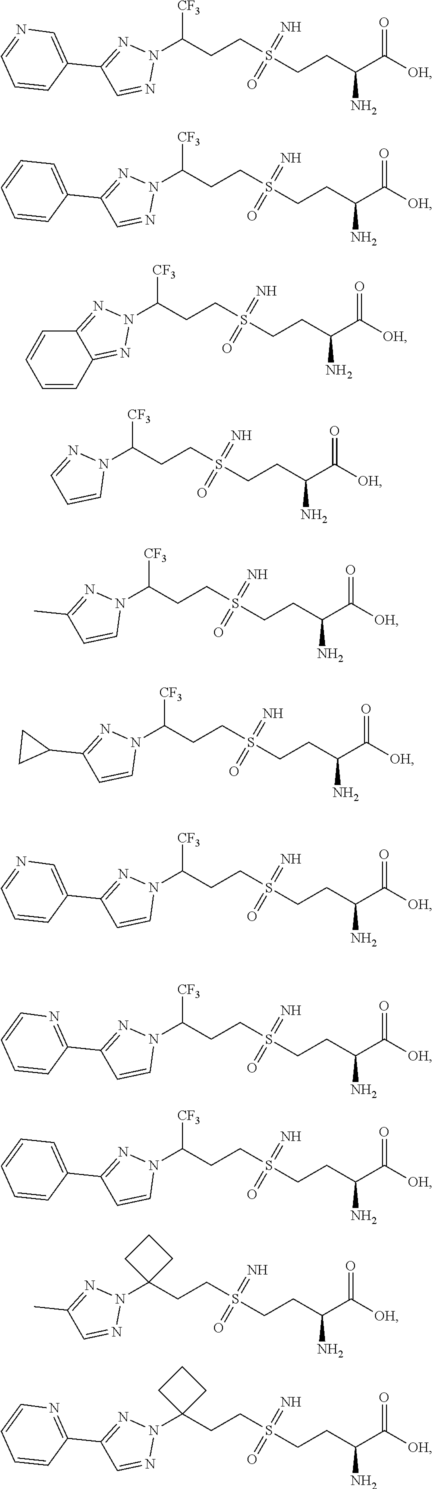

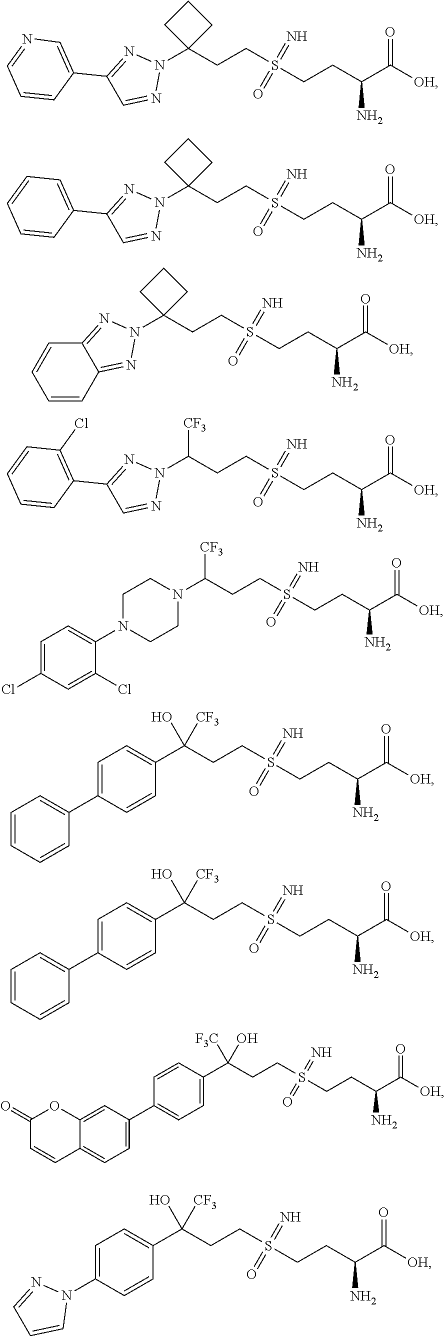

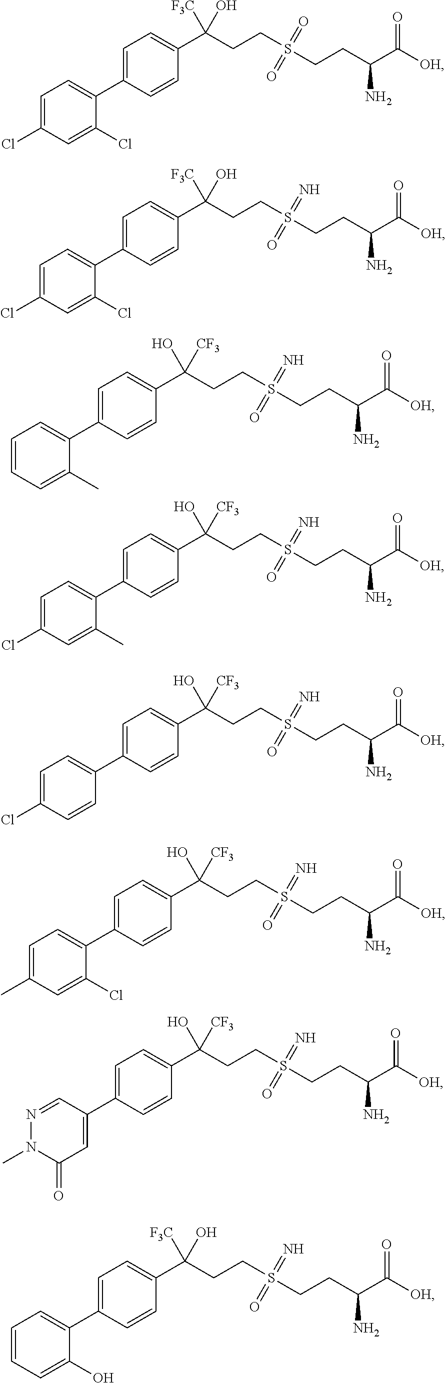

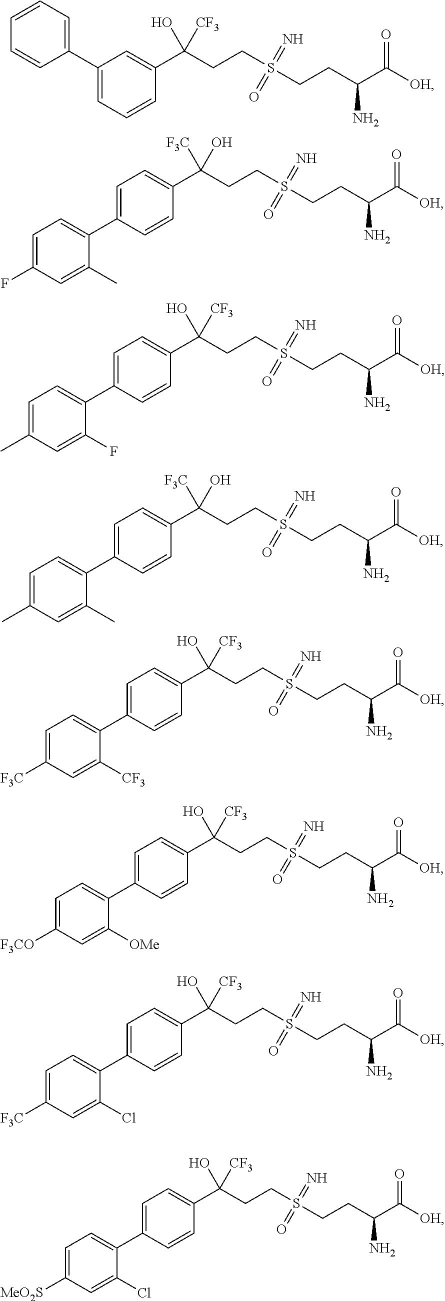

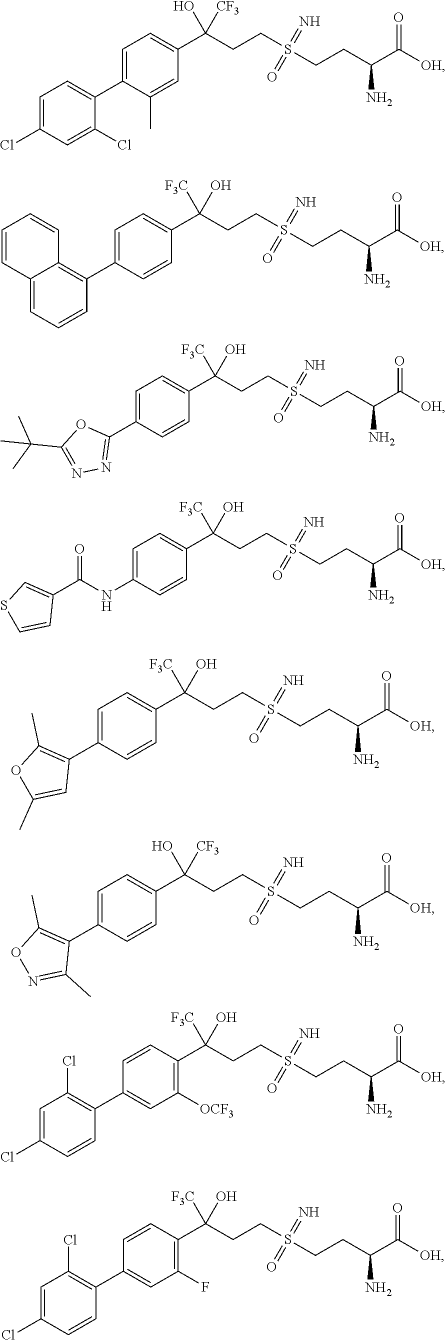

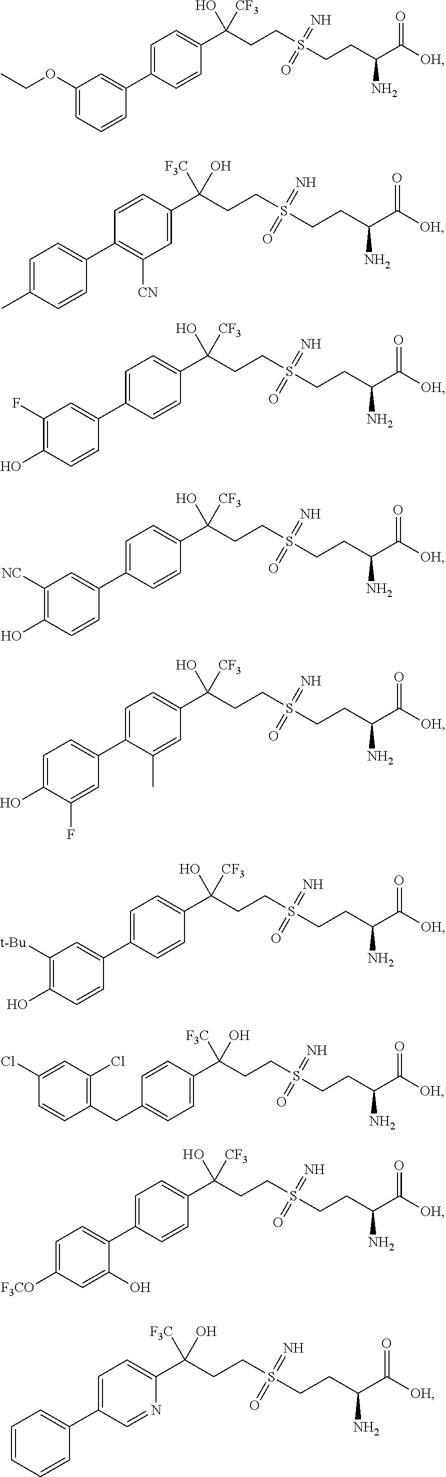

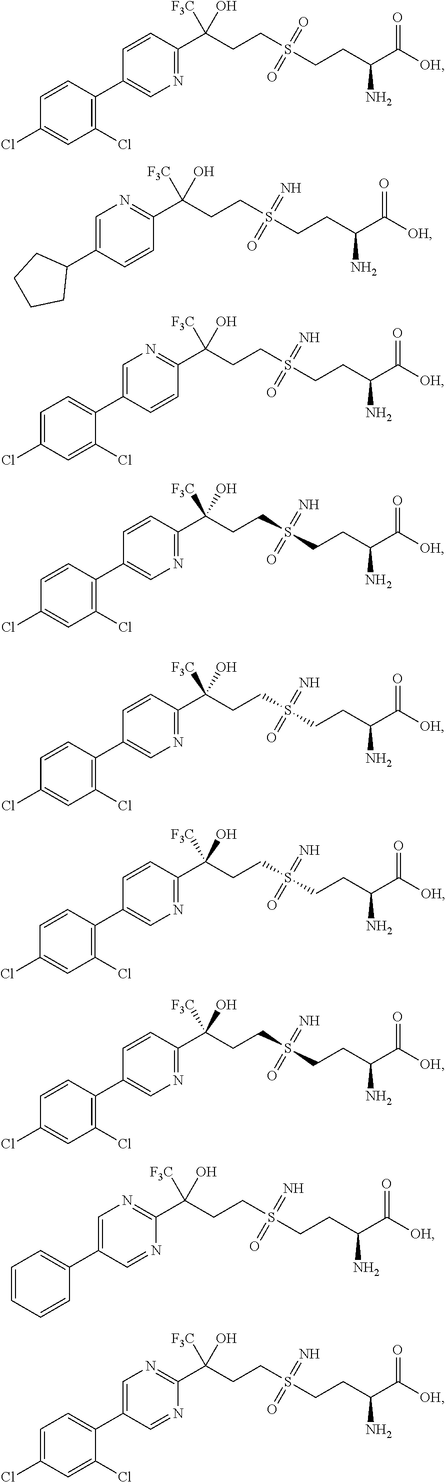

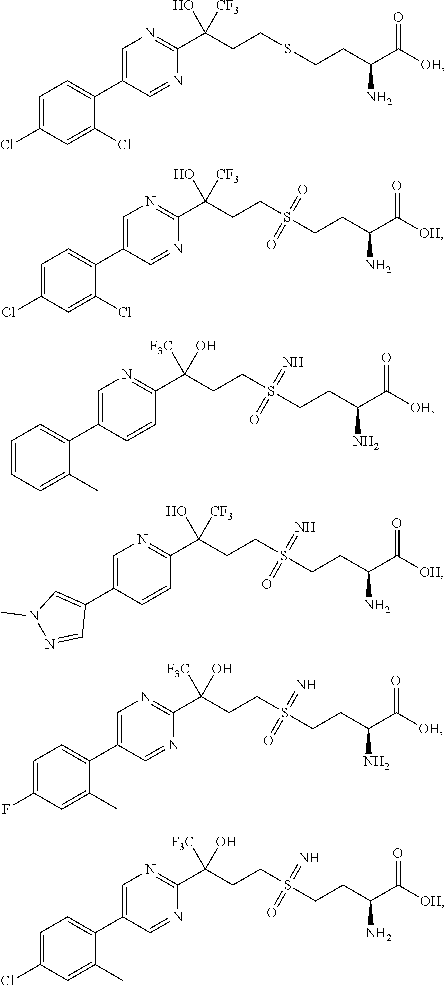 [SMILES: N[C@@H](CCSCCC(O)(c1ncc(-c2ccc(Cl)cc2Cl)cn1)C(F)(F)F)C(=O)O ; N[C@@H](CCS(=O)(=O)CCC(O)(c1ncc(-c2ccc(Cl)cc2Cl)cn1)C(F)(F)F)C(=O)O ; Cc1ccccc1-c1ccc(C(O)(CCS(=N)(=O)CC[C@H](N)C(=O)O)C(F)(F)F)nc1 ; Cn1cc(-c2ccc(C(O)(CCS(=N)(=O)CC[C@H](N)C(=O)O)C(F)(F)F)nc2)cn1 ; Cc1cc(F)ccc1-c1cnc(C(O)(CCS(=N)(=O)CC[C@H](N)C(=O)O)C(F)(F)F)nc1 ; Cc1cc(Cl)ccc1-c1cnc(C(O)(CCS(=N)(=O)CC[C@H](N)C(=O)O)C(F)(F)F)nc1]

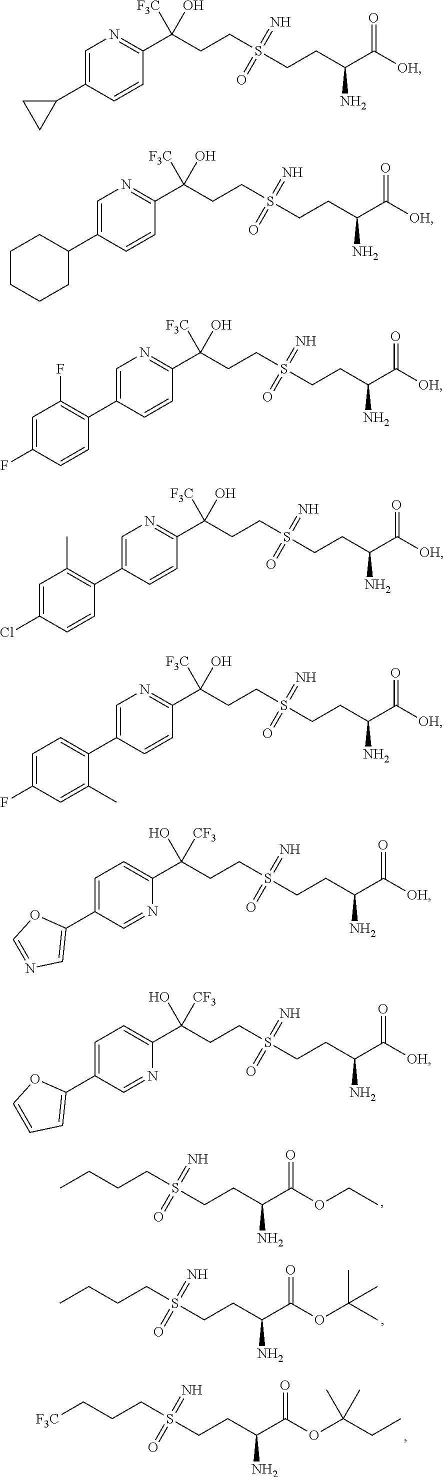

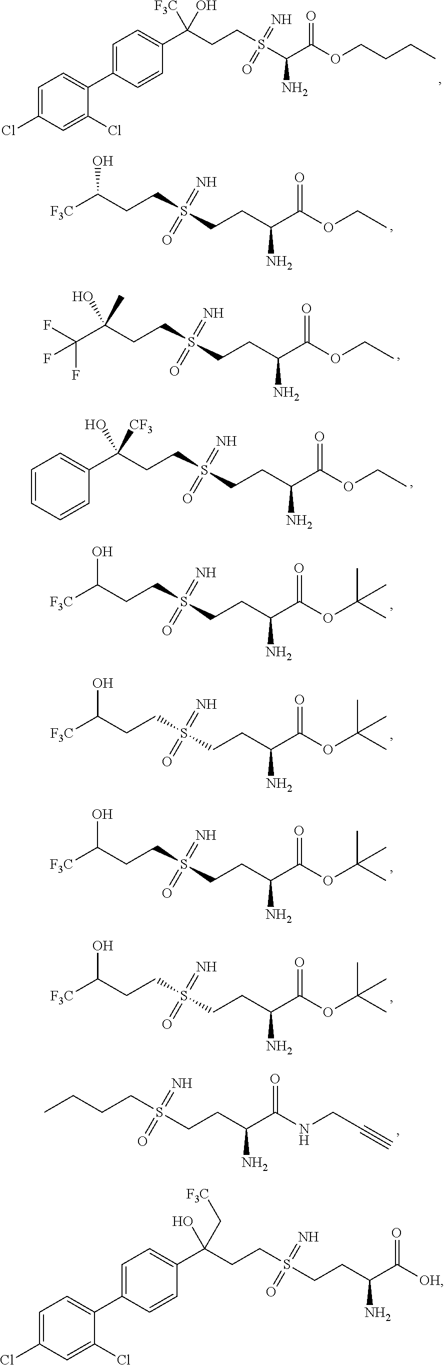

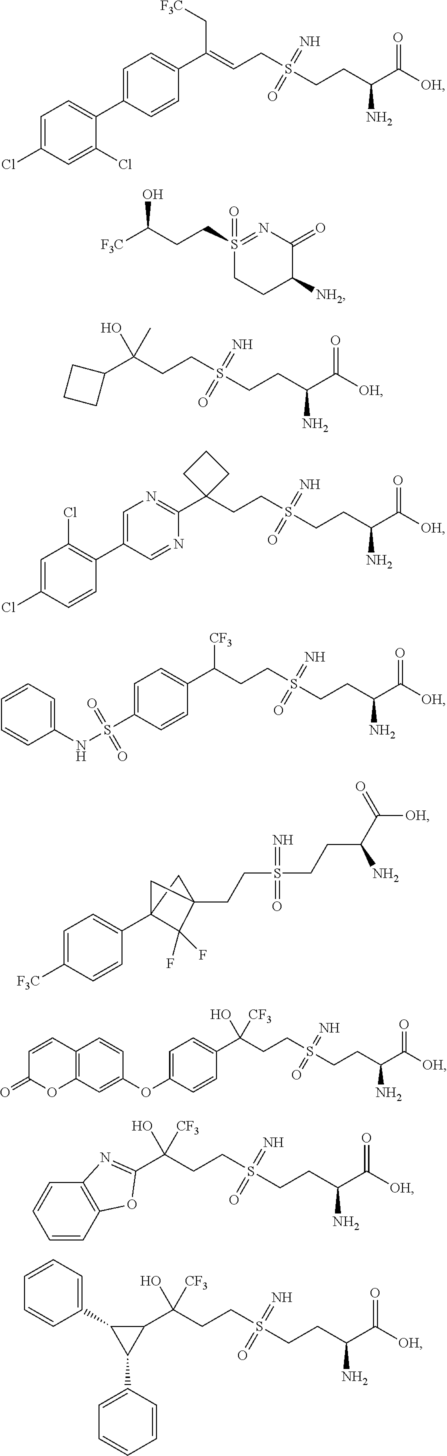

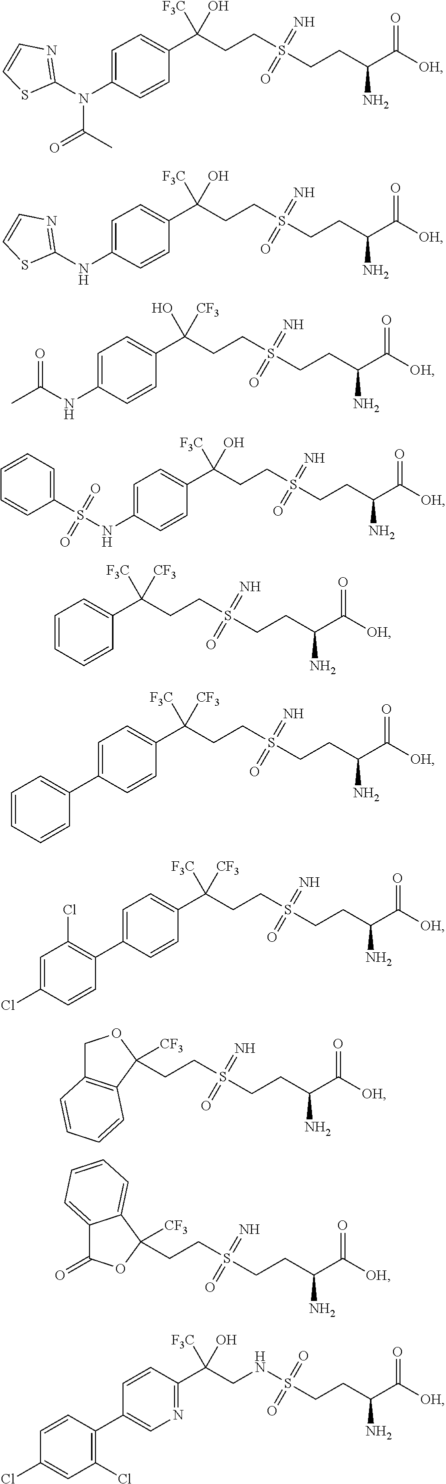

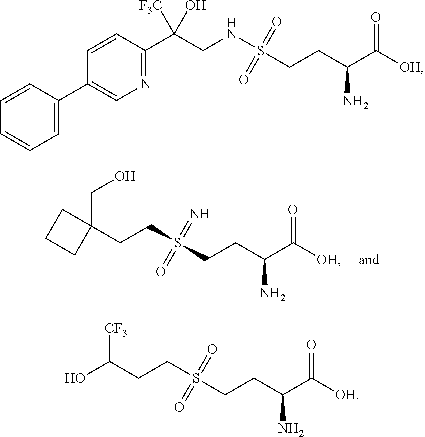 [SMILES: N[C@@H](CCS(=O)(=O)NCC(O)(c1ccc(-c2ccccc2)cn1)C(F)(F)F)C(=O)O ; N=[S@@](=O)(CC[C@H](N)C(=O)O)CCC1(CO)CCC1 ; "and" ; N[C@@H](CCS(=O)(=O)CCC(O)C(F)(F)F)C(=O)O]

42. A compound, a diastereomer or an enantiomer of the compound, or a pharmaceutically acceptable salt of any of the foregoing, or a deuterated derivative of any of the foregoing; wherein the compound is selected from the group consisting of:

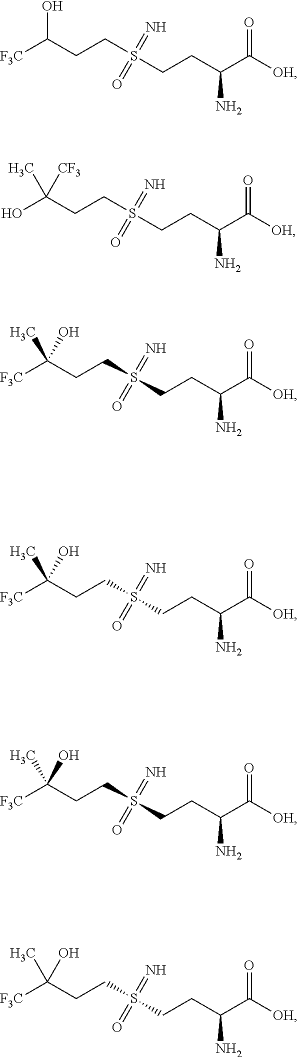 [SMILES: N=S(=O)(CCC(O)C(F)(F)F)CC[C@H](N)C(=O)O ; CC(O)(CCS(=N)(=O)CC[C@H](N)C(=O)O)C(F)(F)F ; C[C@@](O)(CC[S@](=N)(=O)CC[C@H](N)C(=O)O)C(F)(F)F ; C[C@@](O)(CC[S@@](=N)(=O)CC[C@H](N)C(=O)O)C(F)(F)F ; C[C@](O)(CC[S@](=N)(=O)CC[C@H](N)C(=O)O)C(F)(F)F ; CC(O)(CC[S@@](=N)(=O)CC[C@H](N)C(=O)O)C(F)(F)F]

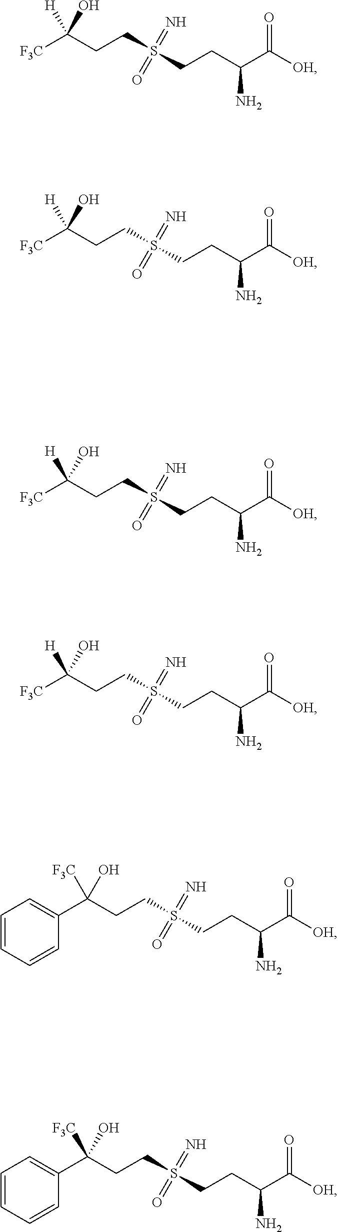 [SMILES: N=[S@@](=O)(CC[C@H](N)C(=O)O)CC[C@H](O)C(F)(F)F ; N=[S@](=O)(CC[C@H](N)C(=O)O)CC[C@H](O)C(F)(F)F ; N=[S@](=O)(CC[C@@H](O)C(F)(F)F)CC[C@H](N)C(=O)O ; N=[S@@](=O)(CC[C@@H](O)C(F)(F)F)CC[C@H](N)C(=O)O ; N=[S@](=O)(CC[C@H](N)C(=O)O)CCC(O)(c1ccccc1)C(F)(F)F ; N=[S@@](=O)(CC[C@H](N)C(=O)O)CC[C@@](O)(c1ccccc1)C(F)(F)F]

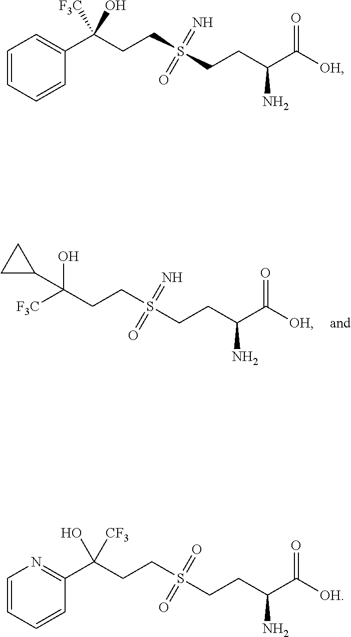 [SMILES: N=[S@@](=O)(CC[C@H](N)C(=O)O)CC[C@](O)(c1ccccc1)C(F)(F)F ; N=S(=O)(CC[C@H](N)C(=O)O)CCC(O)(C1CC1)C(F)(F)F ; N[C@@H](CCS(=O)(=O)CCC(O)(c1ccccn1)C(F)(F)F)C(=O)O]

43. A compound of Formula XVIII: 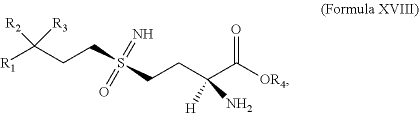
a diastereomer or an enantiomer of the compound of Formula XVIII, or a pharmaceutically acceptable salt of any of the foregoing, or a deuterated derivative of any of the foregoing; wherein in the compound of Formula XVIII:
each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —CH₃, phenyl, —C(O)OR₅, —C(O)NH₂, —O—, —S—, —OH, —NH₂, —NH—, a halogen, —CF₃, —CN, —F, —Cl, —Br, —I,
or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F,
or R₁ and R₂ and R₃ are taken together to form a C₃-C₆ cycloheteroaryl; and
wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

44. A compound of Formula XIX: 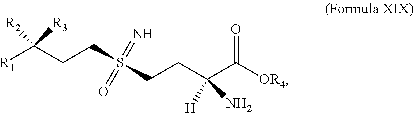
a diastereomer or an enantiomer of the compound of Formula XIX, or a pharmaceutically acceptable salt of any of the foregoing, or a deuterated derivative of any of the foregoing; wherein in the compound of Formula XIX:
each R₁, R₂, or R₃ is independently: H, a C₁-C₁₀ linear or branched alkyl, a C₃-C₆ cycloalkyl, a C₆-C₁₀ aryl, a C₃-C₁₀ heteroaryl, a biphenyl, a halogenated biphenyl, an indole, a triazole, an isothiazole, an oxazoline, a C₁-C₆ linear or branched alkyl ether, —C(O)OR₅, —C(O)NH₂, —O—, —S—, —OH, —NH₂, —NH—, a halogen, —CF₃, —CN, —F, —Cl, —Br, —I,
or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F,
or R₁ and R₂ and R₃ are taken together to form a C₃-C₆ cycloheteroaryl; and
wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, a furan, an oxazoline, a C₃-C₆ heteroaryl, a urea, an anhydride and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

45. A compound of Formula XX: 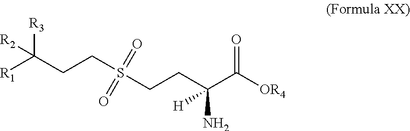
a diastereomer or an enantiomer of the compound of Formula XX, or a pharmaceutically acceptable salt of any of the foregoing, or a deuterated derivative of any of the foregoing; wherein in the compound of Formula XX:
each R₁, R₂, or R₃ is independently: H, a C₁-C₄ linear or branched alkyl, a C₃-C₆ cycloalkyl, a C₆-C₁₀ aryl, a C₅-C₆ heteroaryl, a biphenyl, a halogenated biphenyl, a triazole, an isothiazole, an oxazoline, a C₁-C₆ linear or branched alkyl ether, —C(O)OR₅, —OH, —NH₂, —NH—, a halogen, —CF₃, —CN, —F,
or R₁ and R₂, R₂ and R₃, or R₁ an R₃ are taken together to form a C₃-C₆ cycloalkyl and wherein the C₃-C₆ cycloalkyl may be optionally substituted with a C₁-C₆ linear or branched alkyl, a C₁-C₆ cycloalkyl, a halogen, —CF₃, or —F; and
wherein each R₁, R₂, or R₃ is each independently and optionally substituted by a substituent selected from the group consisting of: a deuterium, a halogen, a fluorine, —CF₃, a chlorine, a C₁-C₁₀ linear or branched chain alkyl, a methyl, an ethyl, a propyl, an iso-propyl, a butyl, an isobutyl, a sec-butyl, a tert-butyl, a C₃-C₆ cycloalkyl, a phenyl, a halogenated phenyl, a biphenyl, a halogenated biphenyl, an isothiazole, a triazole, an oxazoline, a C₃-C₆ heteroaryl, and any combination of these;
wherein R₄ is H, or a C₁-C₆ linear or branched alkyl; and
wherein R₅ is a C₁-C₁₀ linear or branched alkyl optionally substituted with at least one heteroatom, a halogen, or a C₁-C₃ alkyl ether.

46. A pharmaceutical composition comprising the compound of Formula XVIII, XIX, XX, or the compound of any one of claims 40-42; the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier.

47. The pharmaceutical composition of claim 46, that is in unit dose form.

48. The pharmaceutical composition of claim 46 or claim 47, that further comprises an additional active agent or a pharmaceutically acceptable salt thereof.

49. The pharmaceutical composition of claims 46-48, that is in the form of a powder, a tablet, a capsule, a liquid, or a gel.

50. The pharmaceutical composition of any one of claims 46-49, wherein the compound of Formula XVIII, XIX, XX, or the compound of any one of claims 40-42; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg.

51. A kit comprising the pharmaceutical composition of any one of claims 46-50 and a container.

52. The kit of claim 51, wherein the container is a syringe.

53. The kit of claim 51, wherein the container is an IV bag.

54. The kit of claim 51, wherein the container is disposable.

55. The kit of claim 51, wherein the container is a single use container.

56. The kit of claim 51, wherein the container is a resealable container.

57. A method of treating a cancer in a subject, the method comprising administering the pharmaceutical composition of any one of claims 46-50 to the subject in a therapeutically effective amount, thereby treating the cancer.

58. A method of treating a cancer in a subject, the method comprising administering to the subject the compound of Formula XVIII, Formula XIX, Formula XX, or the compound of any one of claims 40-42; the diastereomer or the enantiomer of any of the foregoing, or the pharmaceutically acceptable salt of any of the foregoing, or the deuterated derivative of any of the foregoing; in a therapeutically effective amount, thereby treating the cancer.

59. The method of claim 58, wherein the cancer is a carcinoma, a sarcoma, or a melanoma.

60. The method of claim 58, wherein the cancer is a clear cell renal carcinoma or non-clear cell renal carcinoma.

61. The method of claim 58, wherein the carcinoma is a liver carcinoma.

62. The method of claim 58, wherein the cancer is an SWI/SNF deficient-complex cancer.

63. The method of any one of claims 58 to 62, wherein the administering is selected from the group consisting of: oral, an injection; subcutaneous, intra-tumoral; systemic, local, intravenous, intraperitoneal, intramuscular, and any combination thereof.

64. A method of modulating ferroptosis in a tissue, the method comprising contacting the tissue with the pharmaceutical composition of any one of claims 46-50 in an amount effective to modulate the ferroptosis in the tissue.

65. The method of claim 64, wherein the tissue is comprised in a subject.

66. The method of any one of claims 57-63 or 65, wherein the subject is a subject in need thereof.

67. The method of claim 57 or 58, wherein the subject is a mammal.

68. The method of claim 67, wherein the subject is a human.

69. The method of any one of claims 57-68, wherein the administering or the contacting is: as needed, once per day, twice per day, three times per day, once per week, once per two weeks, once per three weeks, once per month, once every six months, once per year, or for life.

70. The method of any one of claims 57-69, wherein the therapeutically effective amount, or the amount effective, ranges from about 0.001 mg to about 25,000 mg.

71. A method of treating a disease or condition in a subject, the method comprising administering the pharmaceutical composition of any one of claims 46-50 to the subject in a therapeutically effective amount, thereby treating the disease or condition.

72. A method of treating a disease or condition in a subject, the method comprising administering to the subject the compound of any one of claims 46-50; the diastereomer or the enantiomer of any of the foregoing, or the pharmaceutically acceptable salt of any of the foregoing, or the deuterated derivative of any of the foregoing; in a therapeutically effective amount, thereby treating the disease or condition.

73. The method of claim 71 or 72, wherein the disease or condition is a fibrosis or a kidney disorder.
